(19) United States
(12) Patent Application Publication
(10) Pub. No.: US 2024/0381766 A1
(43) Pub. Date: Nov. 14, 2024

(54) ORGANIC LIGHT EMITTING DEVICE

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Young Seok KIM, Daejeon (KR)

(21) Appl. No.: 18/683,462

(22) PCT Filed: Nov. 14, 2022
(86) PCT No.: PCT/KR2022/017853

§ 371 Date: Feb. 13, 2024

(30) Foreign Application Priority Data
Nov. 12, 2021 (KR) 10-2021-0155893
Nov. 11, 2022 (KR) 10-2022-0150849

(51) Int. Cl. H10K 85/60; C07C 211/54; C07C 211/61; C07D 209/86; C07D 307/91; C07D 333/76; C07D 405/12; C07D 409/12; C07D 491/048; C09K 11/06; H10K 50/12; H10K 101/00

(52) U.S. Cl. CPC H10K 85/657; C07C 211/54; C07C 211/61; C07D 209/86; C07D 307/91; C07D 333/76; C07D 405/12; C07D 409/12; C07D 491/048; C09K 11/06; H10K 85/615; H10K 85/622; H10K 85/626; H10K 85/633; H10K 85/636; H10K 85/654; H10K 85/6572; H10K 85/6576; C07C 2603/18; C07C 2603/26; C07C 2603/42; C09K 2211/1003; C09K 2211/1018; H10K 50/12; H10K 2101/90

(57) ABSTRACT

An organic light emitting device having improved driving voltage, efficiency and lifetime. The device includes a light emitting layer containing a compound of Chemical Formulae 1 and 2:

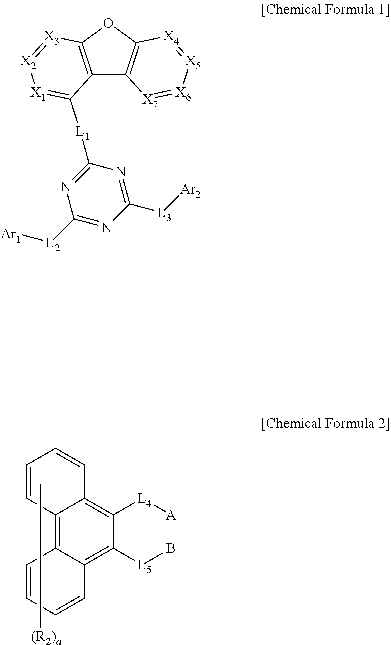

wherein: X₁-X₇ are each independently CR₁ or N, at least one of X₁-X₇ is N, and each R₁ is independently hydrogen, deuterium, or a substituted or unsubstituted C₆₋₆₀ aryl or C₂₋₆₀ heteroaryl containing any one or more of N, O and S; Ar₁ and Ar₂ are each independently a substituted or unsubstituted C₆₋₆₀ aryl or C₂₋₆₀ heteroaryl containing at least one of N, O and S; A and B are each independently hydrogen, deuterium,

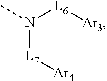

or a substituted or unsubstituted C₆₋₆₀ aryl or C₂₋₆₀ heteroaryl containing at least one of N, O and S, provided that at least one of A and B is

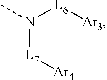

and the other substituents are described in the specification.

### CROSS-REFERENCE TO RELATED APPLICATION(S)

This application is a National Stage Application of International Application No. PCT/KR2022/017853 filed on Nov. 14, 2022, which claims priority to and the benefit of Korean Patent Application No. 10-2021-0155893 filed on Nov. 12, 2021 and Korean Patent Application No. 10-2022-0150849 filed on Nov. 11, 2022 in the Korean Intellectual Property Office, the contents of which are incorporated herein by reference in their entirety.

### TECHNICAL FIELD

The present disclosure relates to an organic light emitting device having improved driving voltage, efficiency and lifetime.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of a new material for an organic material used in the organic light emitting device as described above.

### PRIOR ART LITERATURE

### Patent Literature

Patent Literature 1: Korean Unexamined Patent Publication No. 10-2000-0051826

### BRIEF DESCRIPTION

### Technical Problem

It is an object of the present disclosure to provide an organic light emitting device having improved driving voltage, efficiency and lifetime.

### Technical Solution

The present disclosure provides the following organic light emitting device:
an organic light emitting device comprising:
an anode;
a cathode; and
a light emitting layer interposed between the anode and the cathode,
wherein the light emitting layer includes a compound of the following Chemical Formula 1 and a compound of the following Chemical Formula 2:

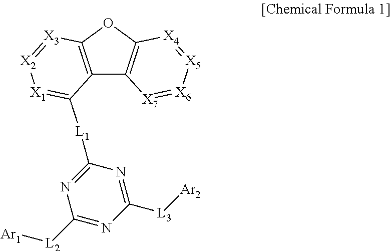

wherein in Chemical Formula 1:
X₁ to X₇ are each independently CR₁ or N, provided that at least one of X₁ to X₇ is N;
each R₁ is independently hydrogen, deuterium, a substituted or unsubstituted C₆₋₆₀ aryl, or a substituted or unsubstituted C₂₋₆₀ heteroaryl containing any one or more heteroatoms selected from the group consisting of N, O and S;
L₁ to L₃ are each independently a single bond, a substituted or unsubstituted C₆₋₆₀ arylene, or a substituted or unsubstituted C₂₋₆₀ heteroarylene containing at least one heteroatom selected from the group consisting of N, O and S; and
Ar₁ and Ar₂ are each independently a substituted or unsubstituted C₆₋₆₀ aryl or a substituted or unsubstituted C₂₋₆₀ heteroaryl containing at least one heteroatom selected from the group consisting of N, O and S;

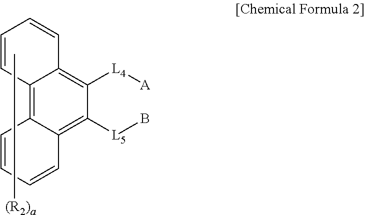

wherein in Chemical Formula 2:
L₄ and L₅ are each independently a single bond, a substituted or unsubstituted C₆₋₆₀ arylene, or a substituted or unsubstituted C₂₋₆₀ heteroarylene containing at least one heteroatom selected from the group consisting of N, O and S;
A and B are each independently hydrogen, deuterium,

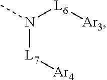

a substituted or unsubstituted C₆₋₆₀ aryl, or a substituted or unsubstituted C₂₋₆₀ heteroaryl containing at least one heteroatom selected from the group consisting of N, O and S, provided that at least one of A and B is

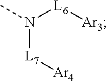

L₆ and L₇ are each independently a single bond, a substituted or unsubstituted C₆₋₆₀ arylene, or a substituted or unsubstituted C₂₋₆₀ heteroarylene containing at least one heteroatom selected from the group consisting of N, O and S;
Ar₃ and Ar₄ are each independently a substituted or unsubstituted C₆₋₆₀ aryl or a substituted or unsubstituted C₂₋₆₀ heteroaryl containing at least one heteroatom selected from the group consisting of N, O and S;
each R₂ is independently hydrogen or deuterium; and
a is an integer of 0 to 8.

### Advantageous Effects

The organic light emitting device described above includes the compound of Chemical Formula 1 and the compound of Chemical Formula 2, and thus, can improve the efficiency, achieve low driving voltage and/or improve lifetime characteristics in the organic light emitting device.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

### DETAILED DESCRIPTION

Hereinafter, embodiments of the present disclosure will be described in more detail to facilitate understanding of the invention.

As used herein, the notation [symbol] or [symbol] means a bond linked to another substituent group.

As used herein, the term “substituted or unsubstituted” means being unsubstituted or substituted with one or more substituents selected from the group consisting of deuterium, a halogen group, a nitrile group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heteroaryl containing at least one of N, O and S atoms, or being unsubstituted or substituted with a substituent to which two or more substituents of the above-exemplified substituents are connected. For example, “a substituent in which two or more substituents are connected” can be a biphenyl group. Namely, a biphenyl group can be an aryl group, or it can be interpreted as a substituent in which two phenyl groups are connected.

In the present disclosure, the carbon number of a carbonyl group is not particularly limited, but is preferably 1 to 40. Specifically, the carbonyl group can be a substituent having the following structural formulas, but is not limited thereto:

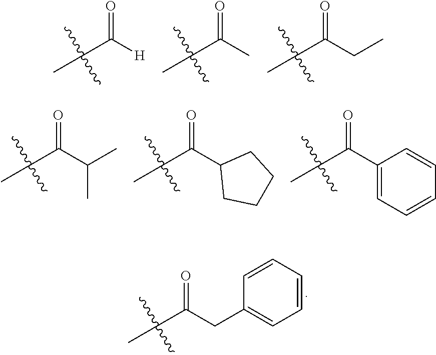

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, the ester group can be a substituent having the following structural formulas, but is not limited thereto:

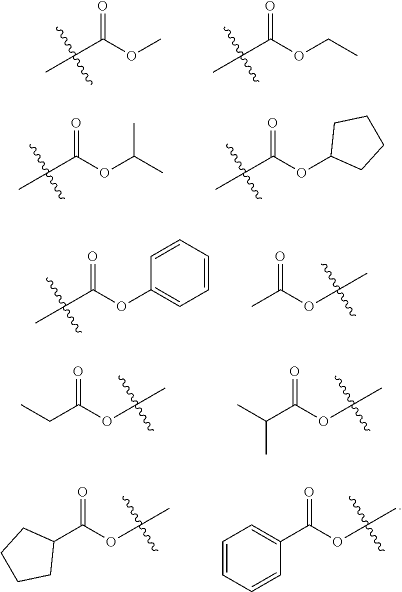

In the present disclosure, the carbon number of an imide group is not particularly limited, but is preferably 1 to 25. Specifically, the imide group can be a substituent having the following structural formulas, but is not limited thereto:

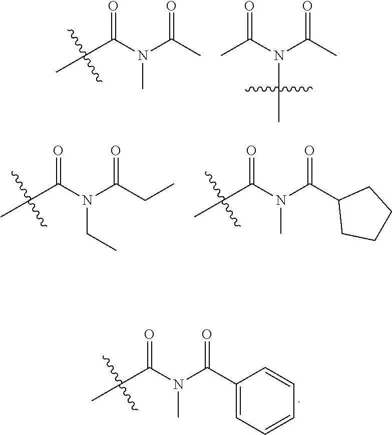

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The aryl group can be a phenyl group, a biphenyl group, a terphenyl group or the like as the monocyclic aryl group, but is not limited thereto. The polycyclic aryl group includes a naphthyl group, an anthracenyl group, a phenanthrenyl group, a pyrenyl group, a perylenyl group, a chrysenyl group, or the like, but is not limited thereto.

In the present disclosure, the fluorenyl group can be substituted, and two substituents can be linked with each other to form a spiro structure. In the case where the fluorenyl group is substituted,

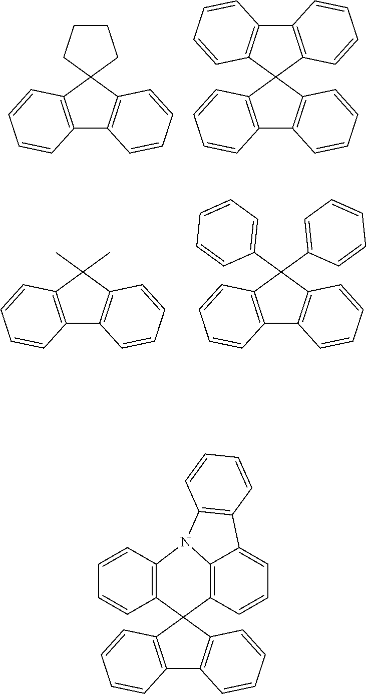

and the like can be formed. However, the structure is not limited thereto.

In the present disclosure, a heteroaryl group is a heteroaryl group containing one or more of O, N, Si and S as a heteroatom, and the carbon number thereof is not particularly limited, but is preferably 2 to 60. According to one embodiment, the carbon number of the heteroaryl group is 6 to 30. According to one embodiment, the carbon number of the heteroaryl group is 6 to 20. Examples of the heteroaryl group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can be applied to the aforementioned description of the heteroaryl group. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heteroaryl group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heteroaryl group can be applied, except that the heteroaryl is not a monovalent group but formed by combining two substituent groups.

Hereinafter, the present disclosure will be described in detail for each configuration.

### Anode and Cathode

An anode and a cathode used in the present disclosure mean electrodes used in an organic light emitting device.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SNO₂:Sb; conductive polymers such as poly(3-methylthiophene), poly[3,4-(ethylene-1,2-dioxy)thiophene] (PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

### Hole Injection Layer

The organic light emitting device according to the present disclosure can further include a hole injection layer on the anode, if necessary.

The hole injection layer is a layer injecting holes from an electrode, and the hole injection material is preferably a compound which has a capability of transporting the holes, has a hole injection effect in the anode and an excellent hole injection effect to the light emitting layer or the light emitting material, prevents movement of an exciton generated in the light emitting layer to the electron injection layer or the electron injection material, and is excellent in the ability to form a thin film. Further, it is preferable that a HOMO (highest occupied molecular orbital) of the hole injection material is between the work function of the anode material and a HOMO of a peripheral organic material layer.

Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

### Hole Transport Layer

The organic light emitting device according to the present disclosure can include a hole transport layer on the anode (or on the hole injection layer if the hole injection layer exists), if necessary.

The hole transport layer is a layer that receives holes from a hole injection layer and transports the holes to the light emitting layer. The hole transport material is suitably a material having large mobility to the holes, which can receive injection of holes from the anode or the hole injection layer and transfer the holes to the light emitting layer.

Specific examples of the hole transport material include an arylamine-based organic material, a conductive polymer, a block copolymer in which a conjugate portion and a non-conjugate portion are present together, and the like, but are not limited thereto.

### Electron Blocking Layer

The organic light emitting device according to the present disclosure includes an electron blocking layer on the hole transport layer, if necessary.

The electron blocking layer means a layer provided between the hole transport layer and the light emitting layer in order to prevent the electrons injected in the cathode from being transferred to the hole transport layer without being recombined in the light emitting layer, which can also be referred to as an electron stopping layer or an electron inhibition layer. The electron blocking layer is preferably a material having the smaller electron affinity than the electron transport layer.

### Light Emitting Layer

The light emitting layer used in the present disclosure is a layer that can emit light in the visible light region by combining holes and electrons transported from the anode and the cathode. Generally, the light emitting layer includes a host material and a dopant material, and in the present disclosure, the compound represented by Chemical Formula 1 and the compound represented by Chemical Formula 2 are included as a host

Preferably, any one of X₁ to X₇ is N, and the rest can be CR₁.

Preferably, the Chemical Formula 1 can be any one of the following Chemical Formula 1-1 to Chemical Formula 1-7:

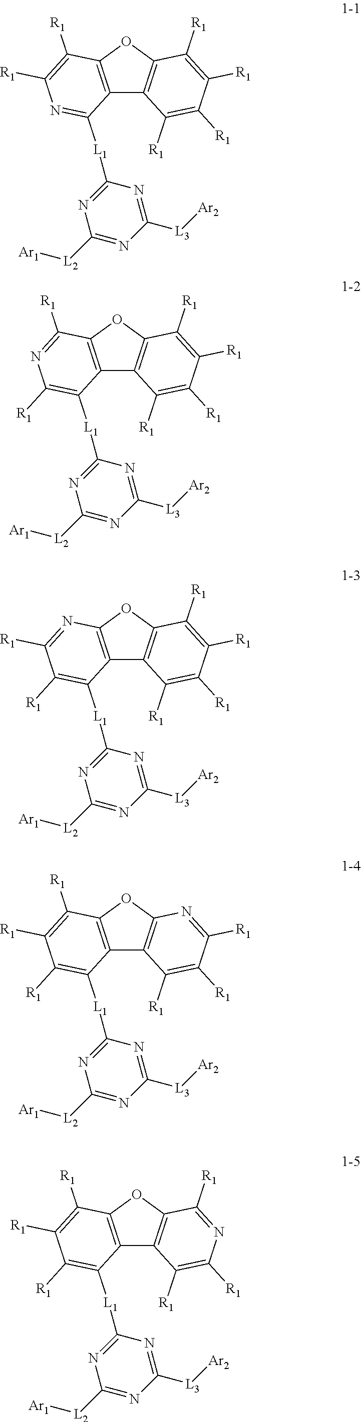

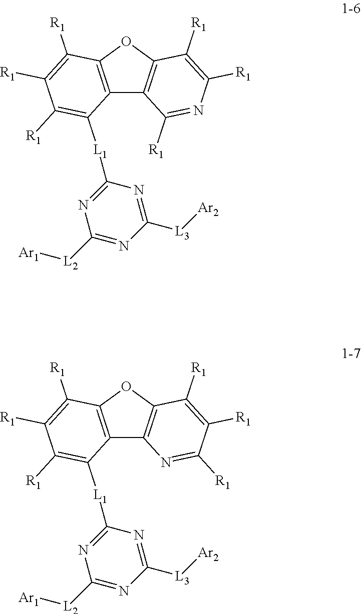

wherein in Chemical Formula-1 to Chemical Formula 1-7,
R₁, L₁ to L₃, Ar₁ and Ar₂ are as defined in Chemical Formula 1.

Preferably, each R₁ can be independently hydrogen, deuterium, a substituted or unsubstituted C₆₋₂₀ aryl, or a substituted or unsubstituted C₂₋₂₀ heteroaryl containing at least one heteroatom selected from the group consisting of N, O and S. More preferably, R₁ is hydrogen, deuterium, phenyl, biphenylyl, naphthyl, carbazolyl, fluoranthenyl, phenanthrenyl, triphenylenyl, benzo[a]carbazolyl, benzo[b]carbazolyl, benzo[c]carbazolyl, dibenzofuranyl, benzo[d]naphtho[1,2-b]furanyl, benzo[d]naphtho[2,3-b]furanyl, benzo[d]naphtho[2,1-b]furanyl, benzo[d]naphtho[1,2-b]thiophenyl, benzo[d]naphtho[2,3-b]thiophenyl, benzo[d]naphtho[2,1-b]thiophenyl, benzo[c]phenanthrenyl, chrysenyl, phenyl naphthyl, or naphthyl phenyl, provided that when the R₁ is not hydrogen or deuterium, R₁ is unsubstituted or substituted with at least one deuterium.

Preferably, one of R₁ is phenyl, biphenylyl, naphthyl, carbazolyl, fluoranthenyl, phenanthrenyl, triphenylenyl, benzo[a]carbazolyl, benzo[b]carbazolyl, benzo[c]carbazolyl, dibenzofuranyl, benzo[d]naphtho[1,2-b]furanyl, benzo[d]naphtho[2,3-b]furanyl, benzo[d]naphtho[2,1-b]furanyl, benzo[d]naphtho[1,2-b]thiophenyl, benzo[d]naphtho[2,3-b]thiophenyl, benzo[d]naphtho[2,1-b]thiophenyl, benzo[c]phenanthrenyl, chrysenyl, phenyl naphthyl, or naphthyl phenyl, and the other R₁s can be hydrogen or deuterium, or each R₁ can be independently hydrogen or deuterium.

Preferably, L₁ to L₃ are each independently a single bond, a substituted or unsubstituted C₆₋₂₀ arylene, or a substituted or unsubstituted C₂₋₂₀ heteroarylene containing at least one heteroatom selected from the group consisting of N, O and S. More preferably, L₁ to L₃ are each independently a single bond, phenylene, naphthalenediyl,

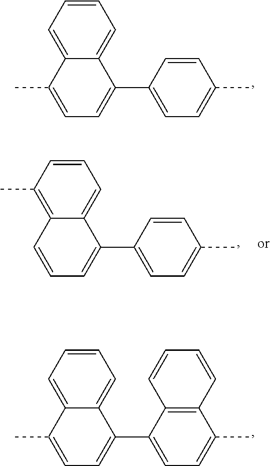

provided that when the L₁ to L₃ are not a single bond, L₁ to L₃ are unsubstituted or substituted with at least one deuterium.

Preferably, L₁ is a single bond, naphthalendiyl,

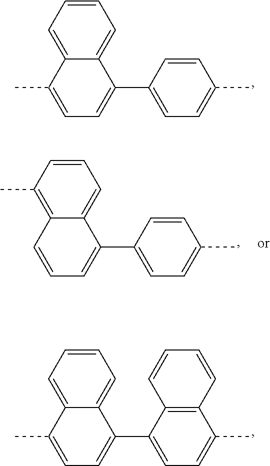

and L₂ and L₃ can be each independently a single bond, phenylene, naphthalendiyl,

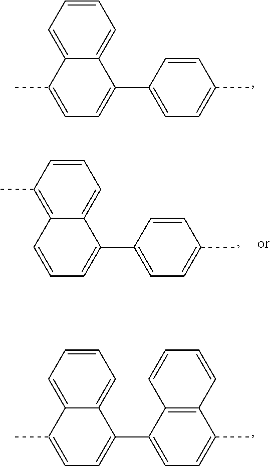

Preferably, Ar₁ and Ar₂ can be each independently a substituted or unsubstituted C₆₋₂₀ aryl or a substituted or unsubstituted C₂₋₂₀ heteroaryl containing at least one heteroatom selected from the group consisting of N, O and S. More preferably, Ar₁ and Ar₂ are each independently phenyl, biphenylyl, terphenylyl, naphthyl, fluoranthenyl, phenanthrenyl, dibenzofuranyl, dibenzothiophenyl, chrysenyl, or benzo[c]phenanthrenyl, provided that the Ar₁ and Ar₂ can be unsubstituted or substituted with at least one deuterium.

Representative examples of the compound of Chemical Formula 1 are as follows:

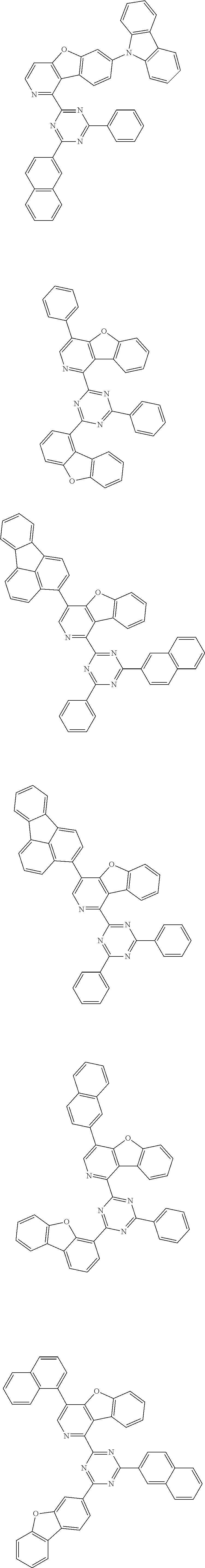

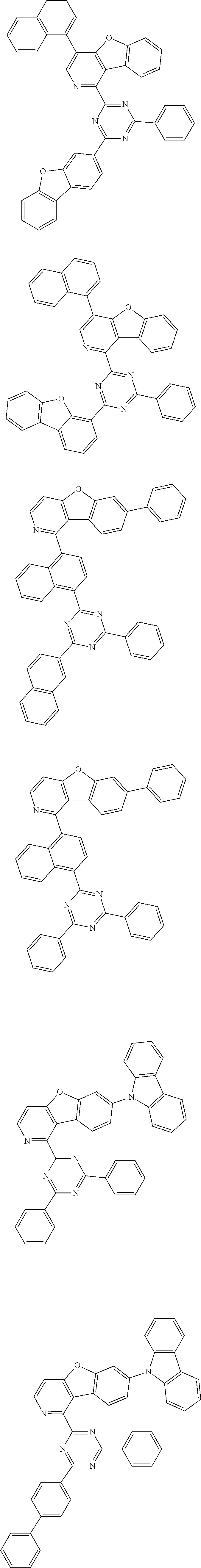

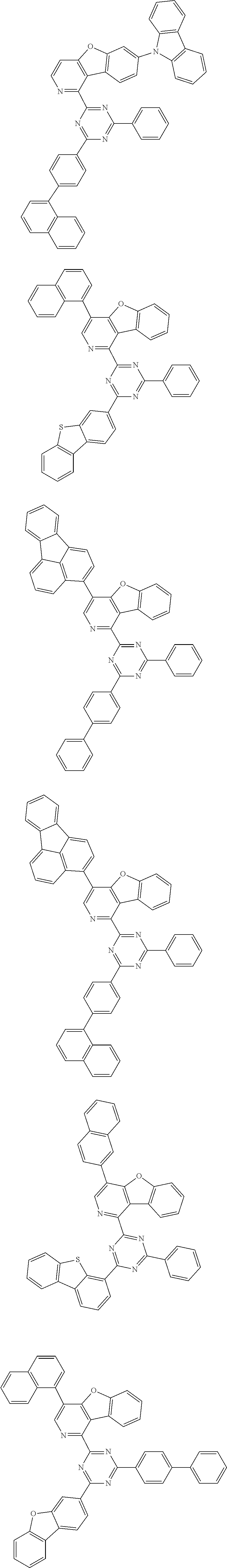

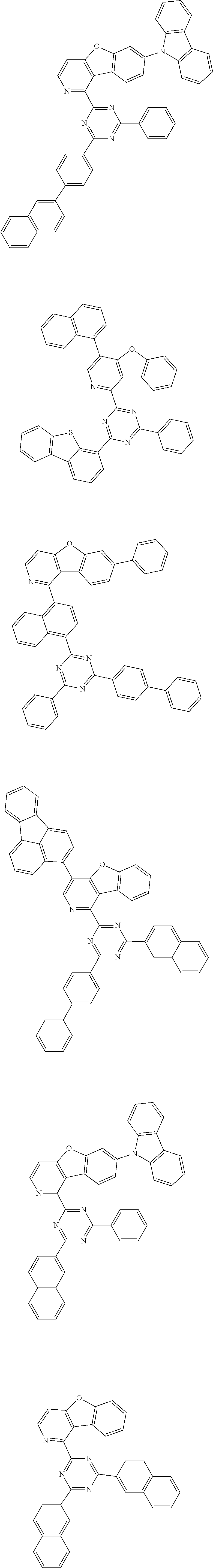

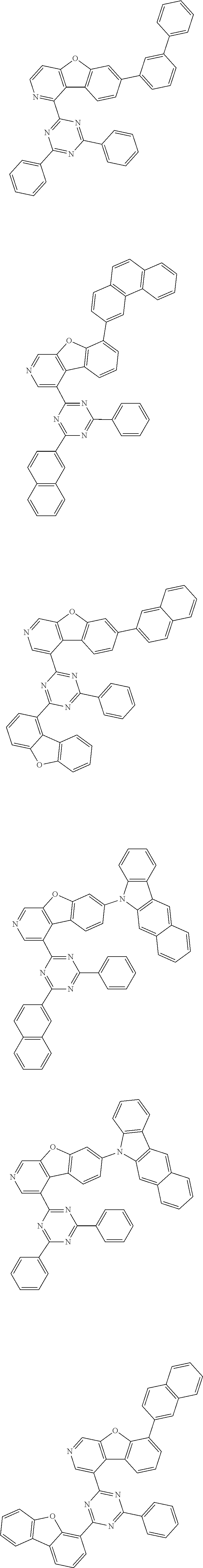 [SMILES: c1ccc(-c2cccc(-c3ccc4c(c3)oc3ccnc(-c5nc(-c6ccccc6)nc(-c6ccccc6)n5)c34)c2)cc1 ; c1ccc(-c2nc(-c3ccc4ccccc4c3)nc(-c3cncc4oc5c(-c6ccc7ccc8ccccc8c7c6)cccc5c34)n2)cc1 ; c1ccc(-c2nc(-c3cccc4oc5ccccc5c34)nc(-c3cncc4oc5cc(-c6ccc7ccccc7c6)ccc5c34)n2)cc1 ; c1ccc(-c2nc(-c3ccc4ccccc4c3)nc(-c3cncc4oc5cc(-n6c7ccccc7c7cc8ccccc8cc76)ccc5c34)n2)cc1 ; c1ccc(-c2nc(-c3ccccc3)nc(-c3cncc4oc5cc(-n6c7ccccc7c7cc8ccccc8cc76)ccc5c34)n2)cc1 ; c1ccc(-c2nc(-c3cccc4c3oc3ccccc34)nc(-c3cncc4oc5c(-c6ccc7ccccc7c6)cccc5c34)n2)cc1]

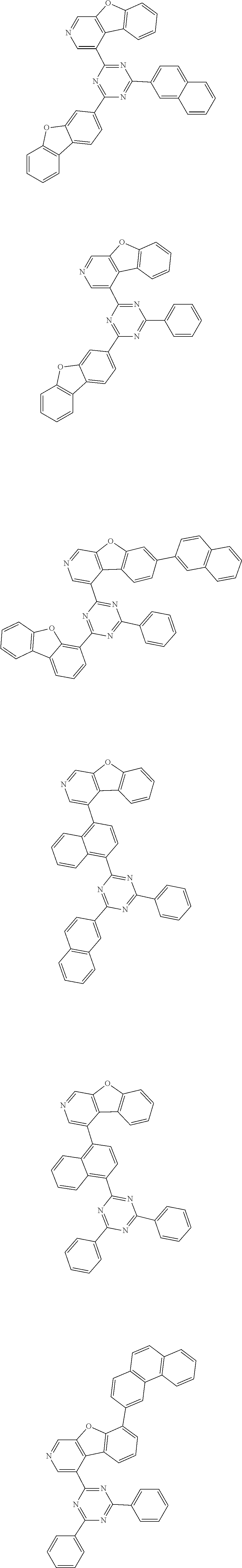 [SMILES: c1ccc2cc(-c3nc(-c4ccc5c(c4)oc4ccccc45)nc(-c4cncc5oc6ccccc6c45)n3)ccc2c1 ; c1ccc(-c2nc(-c3ccc4c(c3)oc3ccccc34)nc(-c3cncc4oc5ccccc5c34)n2)cc1 ; c1ccc(-c2nc(-c3cccc4c3oc3ccccc34)nc(-c3cncc4oc5cc(-c6ccc7ccccc7c6)ccc5c34)n2)cc1 ; c1ccc(-c2nc(-c3ccc4ccccc4c3)nc(-c3ccc(-c4cncc5oc6ccccc6c45)c4ccccc34)n2)cc1 ; c1ccc(-c2nc(-c3ccccc3)nc(-c3ccc(-c4cncc5oc6ccccc6c45)c4ccccc34)n2)cc1 ; c1ccc(-c2nc(-c3ccccc3)nc(-c3cncc4oc5c(-c6ccc7ccc8ccccc8c7c6)cccc5c34)n2)cc1]

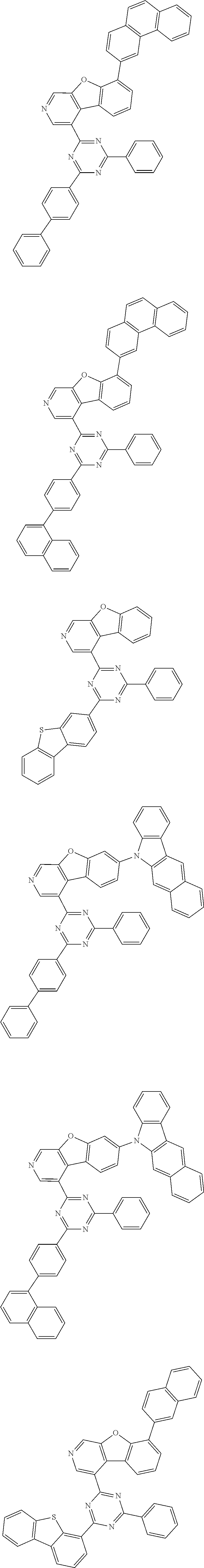

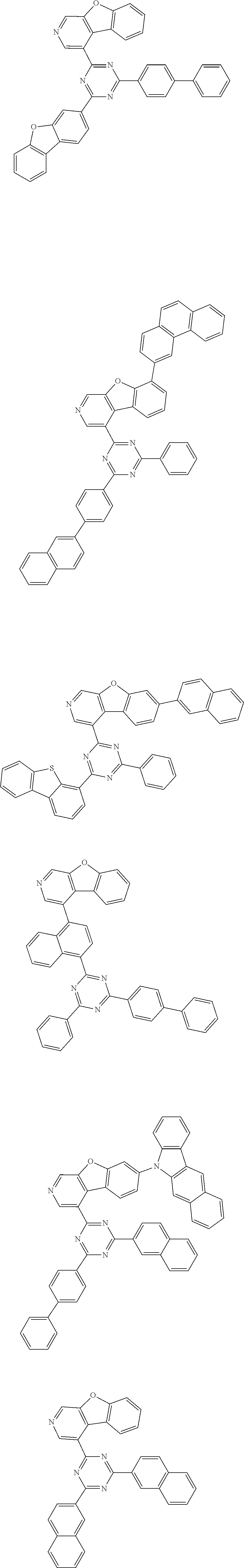

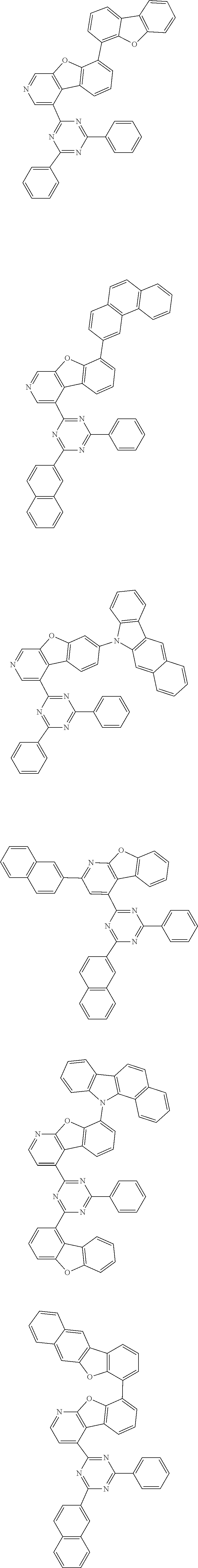

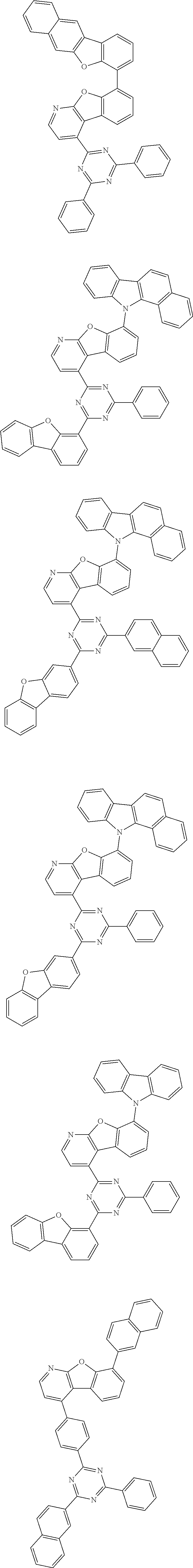

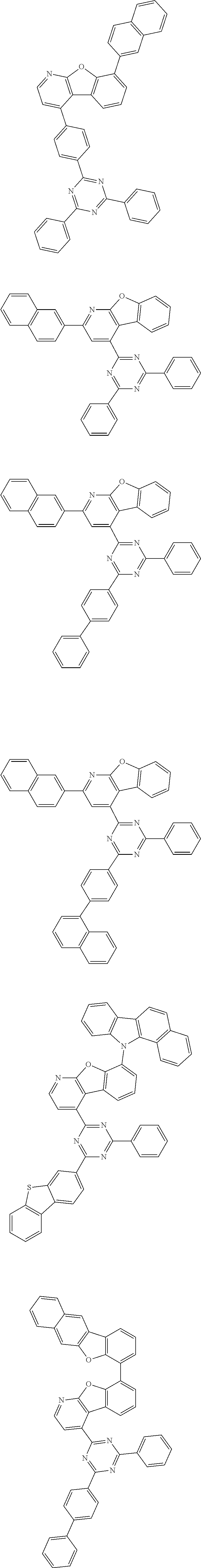

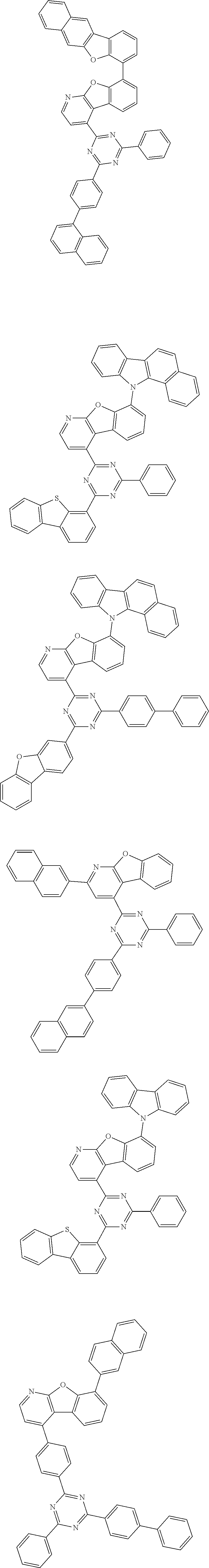

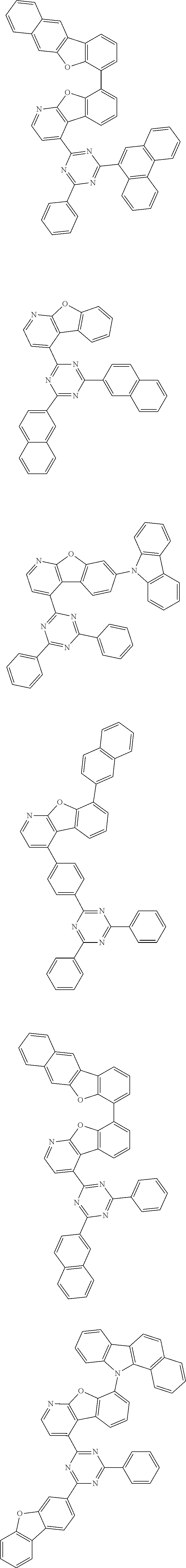

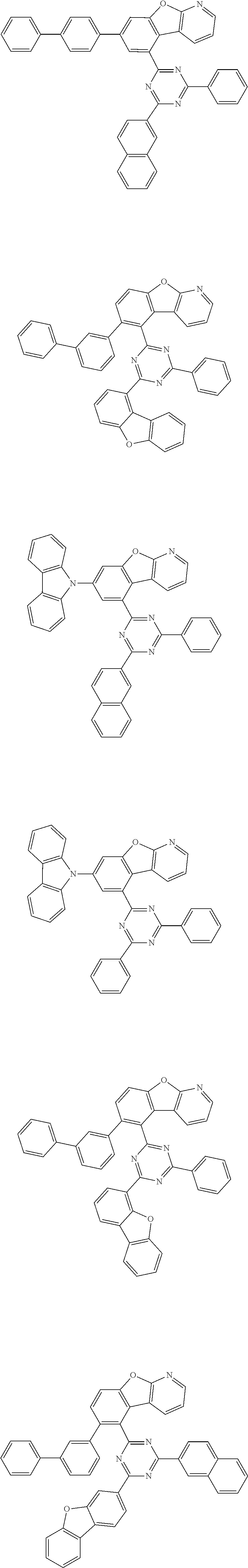

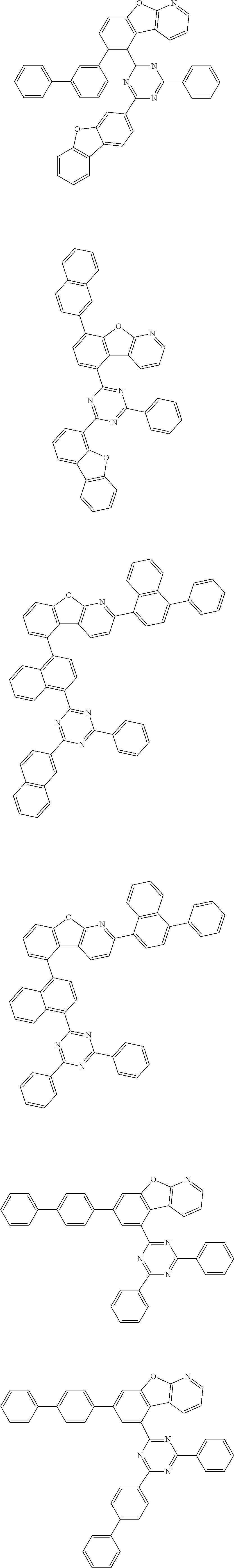

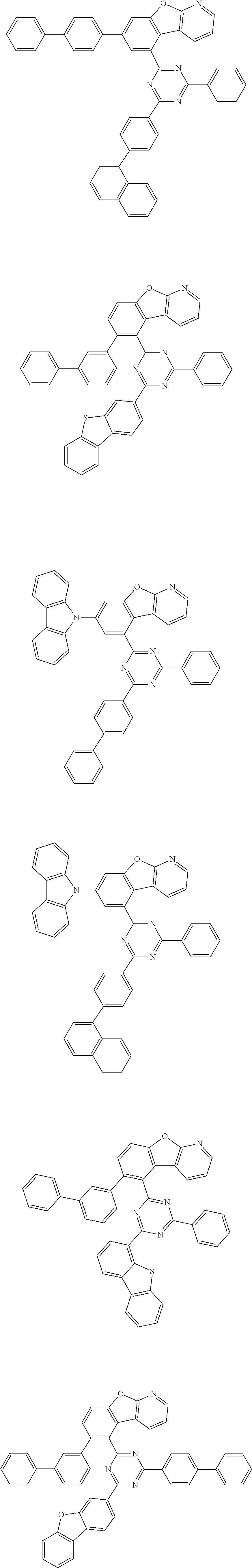

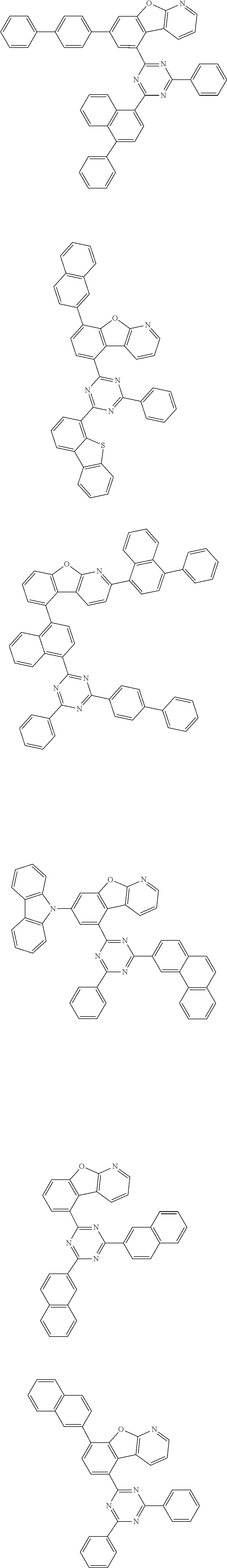

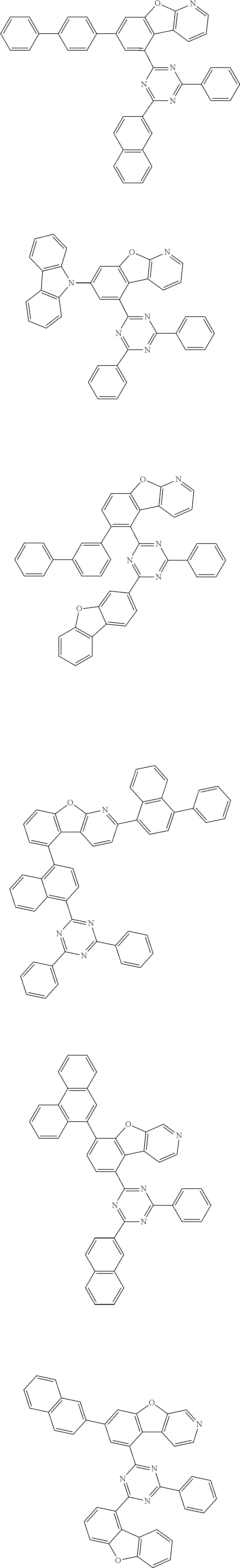

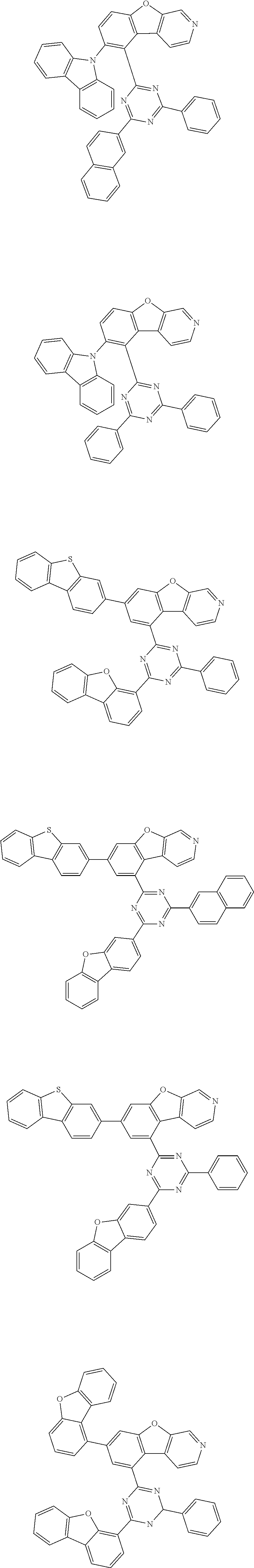

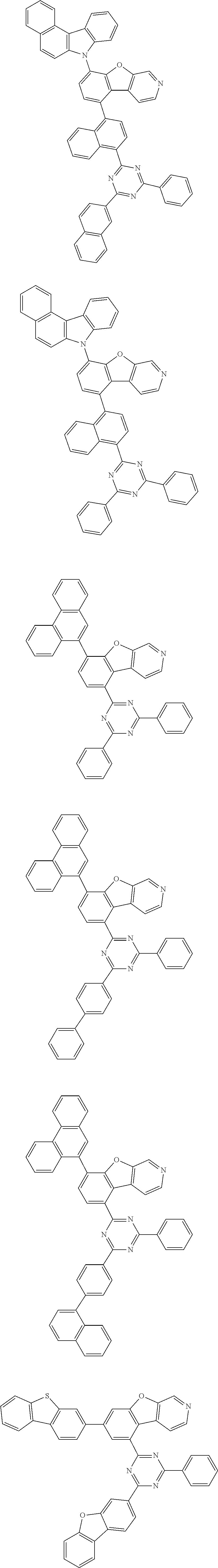

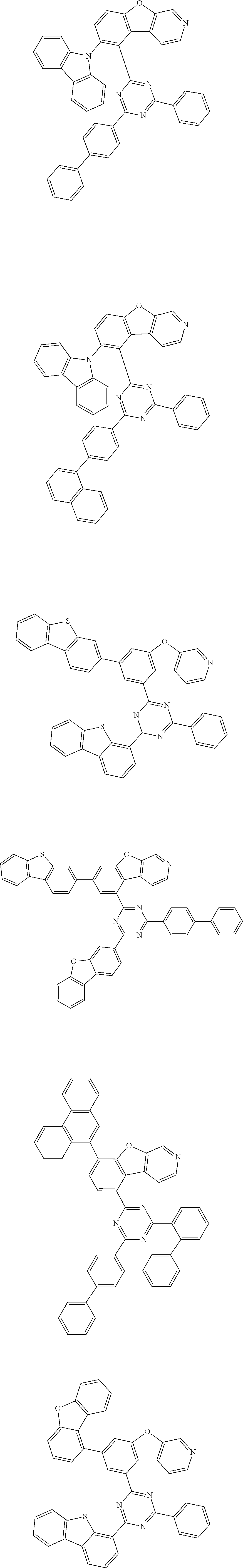

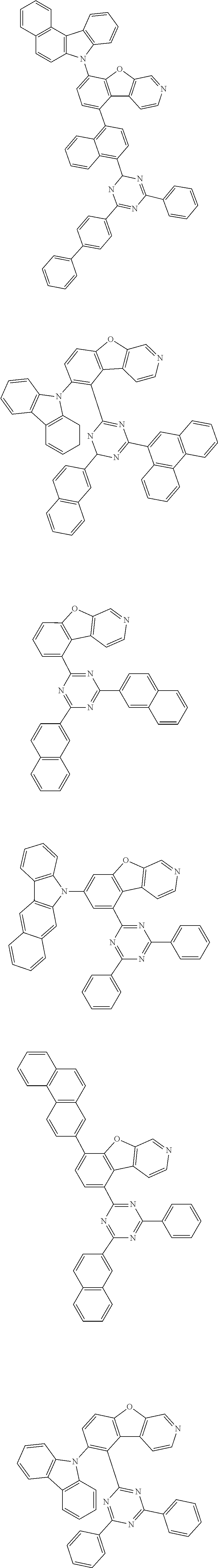

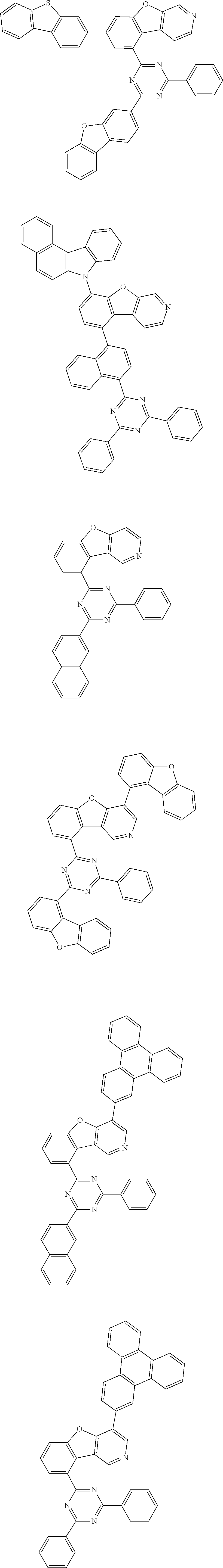

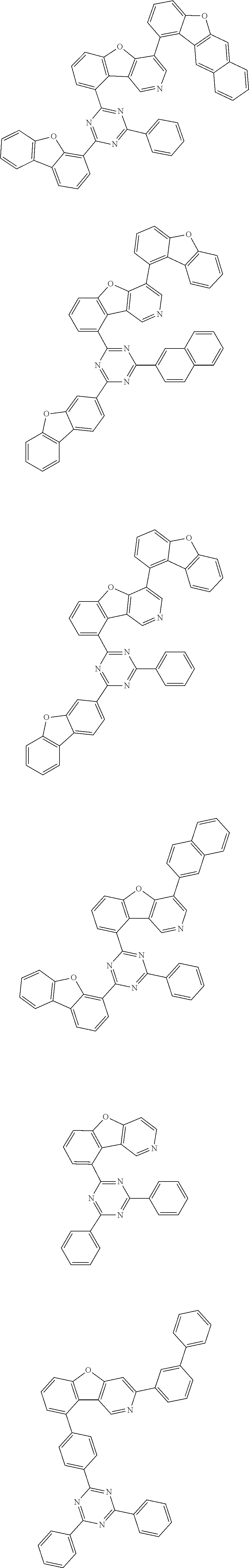

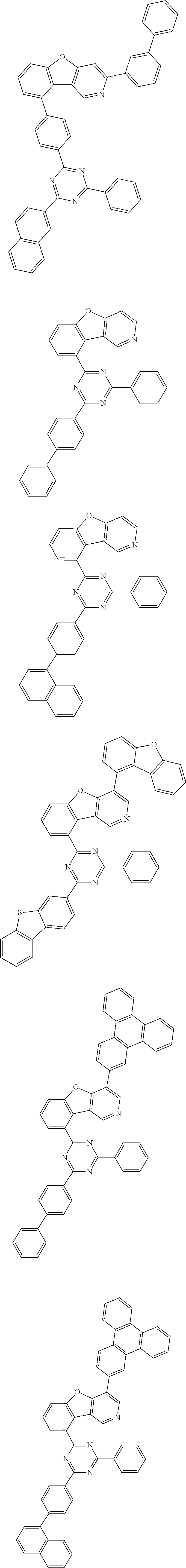

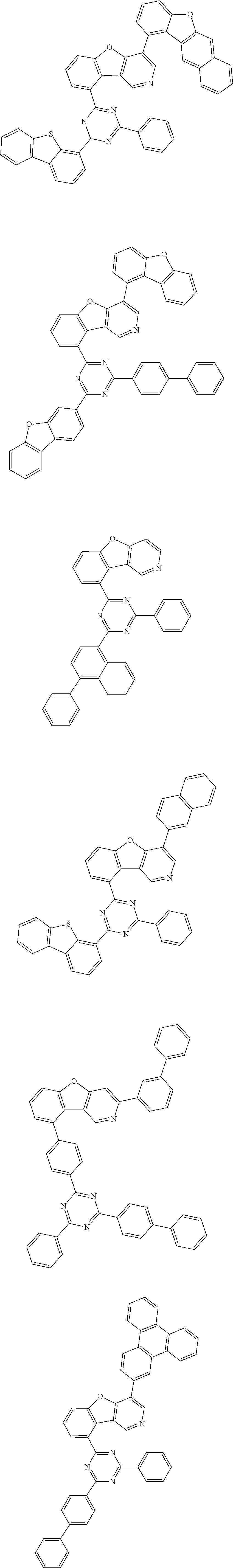

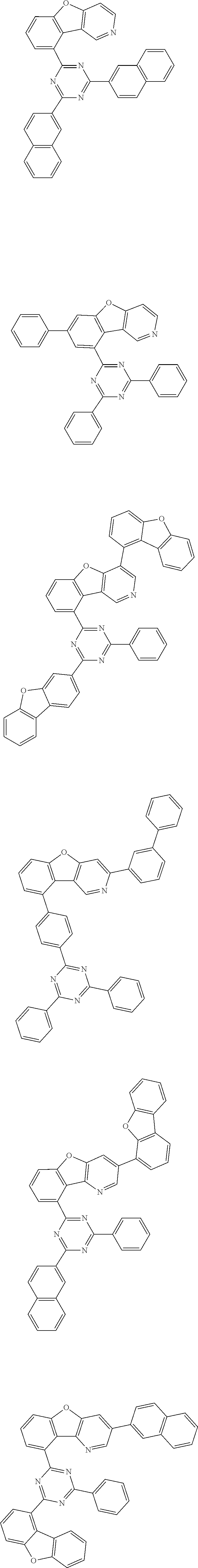 [SMILES: c1ccc2cc(-c3nc(-c4ccc5ccccc5c4)nc(-c4cccc5oc6ccncc6c45)n3)ccc2c1 ; c1ccc(-c2cc(-c3nc(-c4ccccc4)nc(-c4ccccc4)n3)c3c(c2)oc2ccncc23)cc1 ; c1ccc(-c2nc(-c3ccc4c(c3)oc3ccccc34)nc(-c3cccc4oc5c(-c6cccc7oc8ccccc8c67)cncc5c34)n2)cc1 ; c1ccc(-c2cccc(-c3cc4oc5cccc(-c6ccc(-c7nc(-c8ccccc8)nc(-c8ccccc8)n7)cc6)c5c4cn3)c2)cc1 ; c1ccc(-c2nc(-c3ccc4ccccc4c3)nc(-c3cccc4oc5cc(-c6cccc7c6oc6ccccc67)cnc5c34)n2)cc1 ; c1ccc(-c2nc(-c3cccc4oc5ccccc5c34)nc(-c3cccc4oc5cc(-c6ccc7ccccc7c6)cnc5c34)n2)cc1]

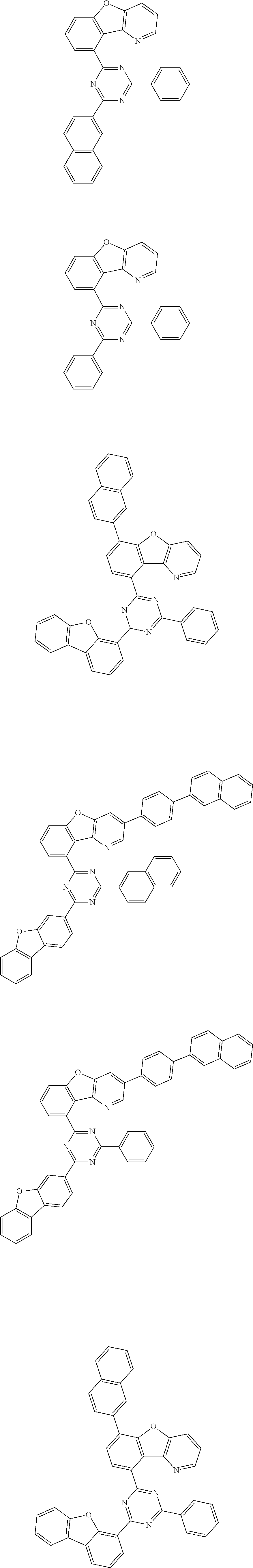

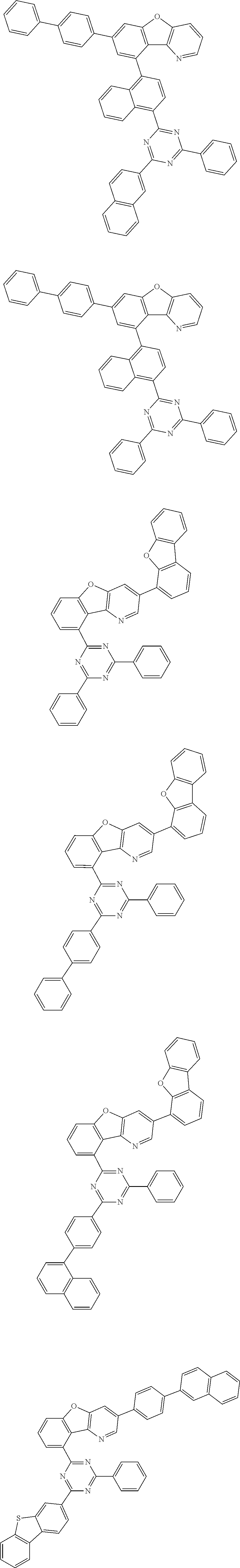

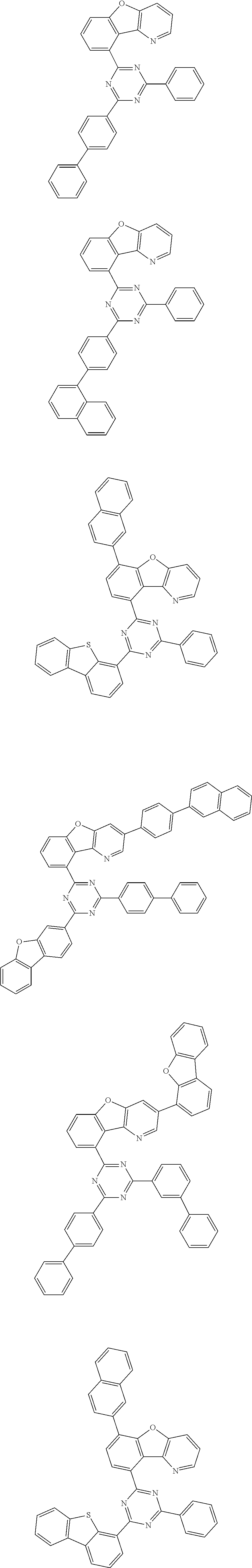

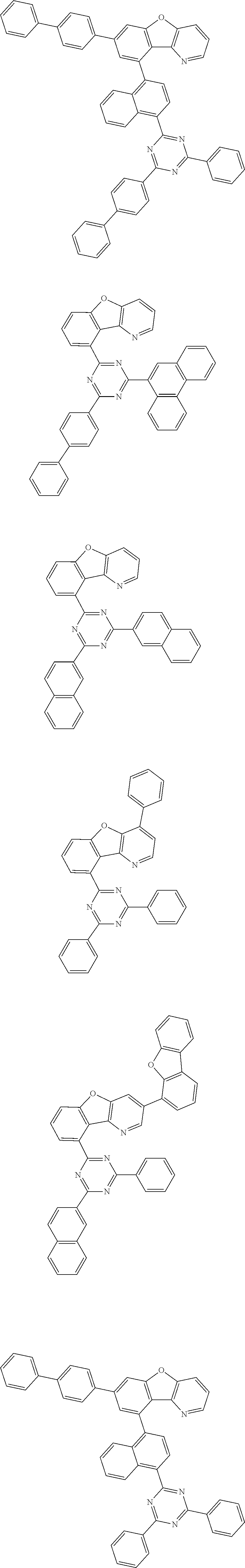 [SMILES: c1ccc(-c2ccc(-c3cc(-c4ccc(-c5nc(-c6ccccc6)nc(-c6ccc(-c7ccccc7)cc6)n5)c5ccccc45)c4c(c3)oc3cccnc34)cc2)cc1 ; c1ccc(-c2ccc(-c3nc(-c4cc5ccccc5c5ccccc45)nc(-c4cccc5oc6cccnc6c45)n3)cc2)cc1 ; c1ccc2cc(-c3nc(-c4ccc5ccccc5c4)nc(-c4cccc5oc6cccnc6c45)n3)ccc2c1 ; c1ccc(-c2nc(-c3ccccc3)nc(-c3cccc4oc5c(-c6ccccc6)ccnc5c34)n2)cc1 ; c1ccc(-c2nc(-c3ccc4ccccc4c3)nc(-c3cccc4oc5cc(-c6cccc7c6oc6ccccc67)cnc5c34)n2)cc1 ; c1ccc(-c2ccc(-c3cc(-c4ccc(-c5nc(-c6ccccc6)nc(-c6ccccc6)n5)c5ccccc45)c4c(c3)oc3cccnc34)cc2)cc1]

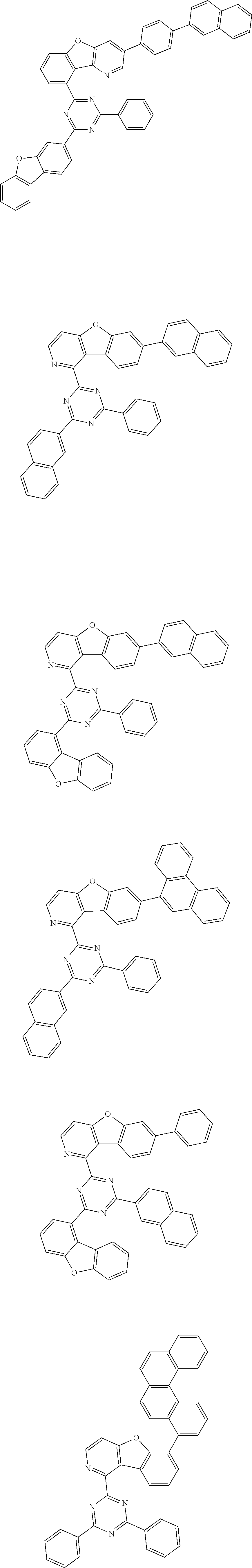

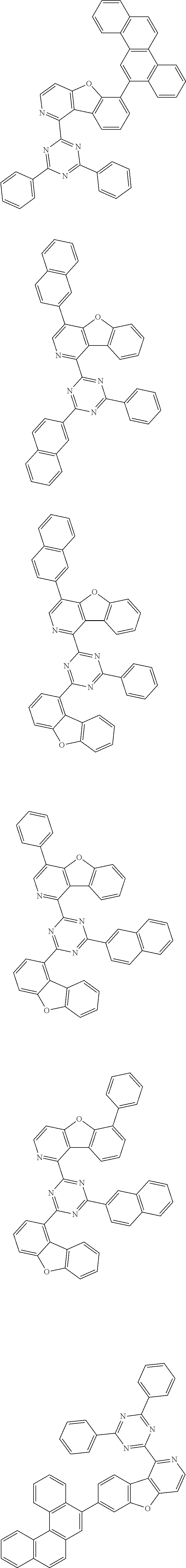 [SMILES: c1ccc(-c2nc(-c3ccccc3)nc(-c3nccc4oc5c(-c6cc7c8ccccc8ccc7c7ccccc67)cccc5c34)n2)cc1 ; c1ccc(-c2nc(-c3ccc4ccccc4c3)nc(-c3ncc(-c4ccc5ccccc5c4)c4oc5ccccc5c34)n2)cc1 ; c1ccc(-c2nc(-c3cccc4oc5ccccc5c34)nc(-c3ncc(-c4ccc5ccccc5c4)c4oc5ccccc5c34)n2)cc1 ; c1ccc(-c2cnc(-c3nc(-c4ccc5ccccc5c4)nc(-c4cccc5oc6ccccc6c45)n3)c3c2oc2ccccc23)cc1 ; c1ccc(-c2cccc3c2oc2ccnc(-c4nc(-c5ccc6ccccc6c5)nc(-c5cccc6oc7ccccc7c56)n4)c23)cc1 ; c1ccc(-c2nc(-c3ccccc3)nc(-c3nccc4oc5cc(-c6cc7ccc8ccccc8c7c7ccccc67)ccc5c34)n2)cc1]

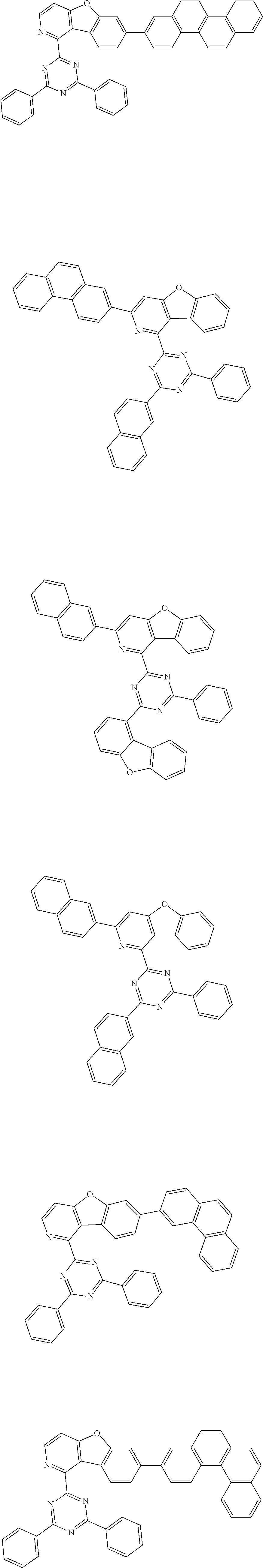 [SMILES: c1ccc(-c2nc(-c3ccccc3)nc(-c3nccc4oc5cc(-c6ccc7c(ccc8c9ccccc9ccc78)c6)ccc5c34)n2)cc1 ; c1ccc(-c2nc(-c3ccc4ccccc4c3)nc(-c3nc(-c4ccc5c(ccc6ccccc65)c4)cc4oc5ccccc5c34)n2)cc1 ; c1ccc(-c2nc(-c3cccc4oc5ccccc5c34)nc(-c3nc(-c4ccc5ccccc5c4)cc4oc5ccccc5c34)n2)cc1 ; c1ccc(-c2nc(-c3ccc4ccccc4c3)nc(-c3nc(-c4ccc5ccccc5c4)cc4oc5ccccc5c34)n2)cc1 ; c1ccc(-c2nc(-c3ccccc3)nc(-c3nccc4oc5cc(-c6ccc7ccc8ccccc8c7c6)ccc5c34)n2)cc1 ; c1ccc(-c2nc(-c3ccccc3)nc(-c3nccc4oc5cc(-c6ccc7c(ccc8ccc9ccccc9c87)c6)ccc5c34)n2)cc1]

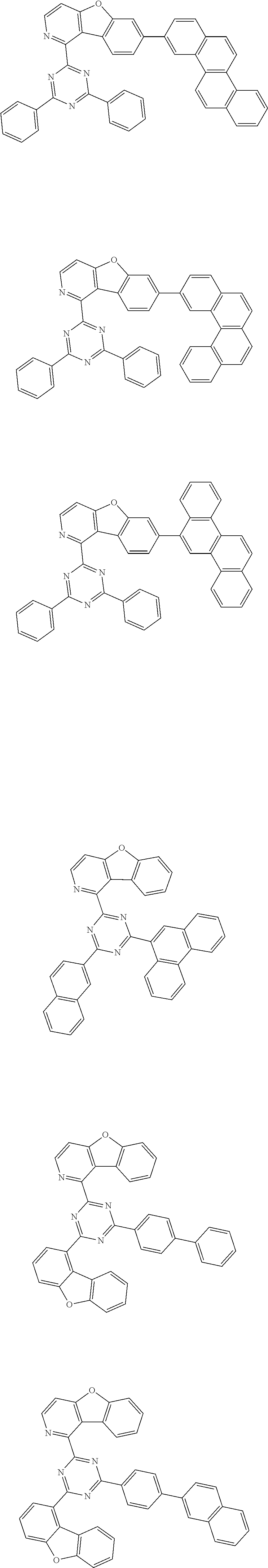 [SMILES: c1ccc(-c2nc(-c3ccccc3)nc(-c3nccc4oc5cc(-c6ccc7ccc8c9ccccc9ccc8c7c6)ccc5c34)n2)cc1 ; c1ccc(-c2nc(-c3ccccc3)nc(-c3nccc4oc5cc(-c6ccc7ccc8ccc9ccccc9c8c7c6)ccc5c34)n2)cc1 ; c1ccc(-c2nc(-c3ccccc3)nc(-c3nccc4oc5cc(-c6cc7c8ccccc8ccc7c7ccccc67)ccc5c34)n2)cc1 ; c1ccc2cc(-c3nc(-c4cc5ccccc5c5ccccc45)nc(-c4nccc5oc6ccccc6c45)n3)ccc2c1 ; c1ccc(-c2ccc(-c3nc(-c4cccc5oc6ccccc6c45)nc(-c4nccc5oc6ccccc6c45)n3)cc2)cc1 ; c1ccc2cc(-c3ccc(-c4nc(-c5cccc6oc7ccccc7c56)nc(-c5nccc6oc7ccccc7c56)n4)cc3)ccc2c1]

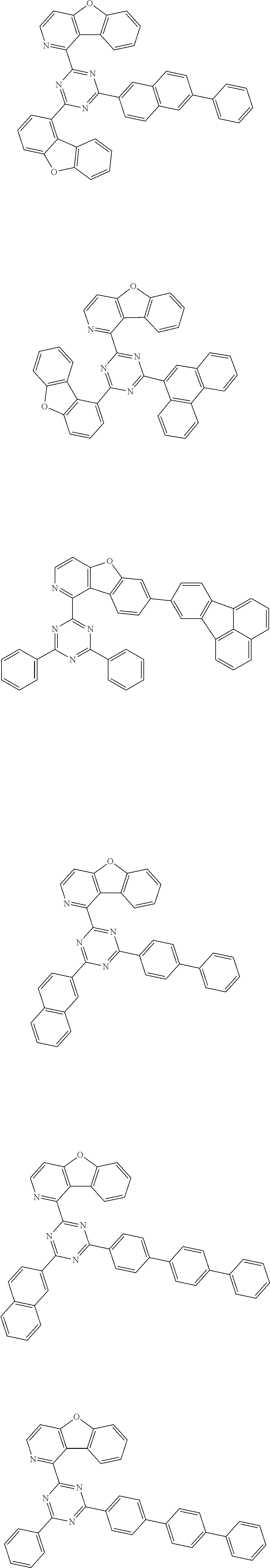 [SMILES: c1ccc(-c2ccc3cc(-c4nc(-c5cccc6oc7ccccc7c56)nc(-c5nccc6oc7ccccc7c56)n4)ccc3c2)cc1 ; c1ccc2c(c1)cc(-c1nc(-c3cccc4oc5ccccc5c34)nc(-c3nccc4oc5ccccc5c34)n1)c1ccccc12 ; c1ccc(-c2nc(-c3ccccc3)nc(-c3nccc4oc5cc(-c6ccc7c(c6)-c6cccc8cccc-7c68)ccc5c34)n2)cc1 ; c1ccc(-c2ccc(-c3nc(-c4ccc5ccccc5c4)nc(-c4nccc5oc6ccccc6c45)n3)cc2)cc1 ; c1ccc(-c2ccc(-c3ccc(-c4nc(-c5ccc6ccccc6c5)nc(-c5nccc6oc7ccccc7c56)n4)cc3)cc2)cc1 ; c1ccc(-c2ccc(-c3ccc(-c4nc(-c5ccccc5)nc(-c5nccc6oc7ccccc7c56)n4)cc3)cc2)cc1]

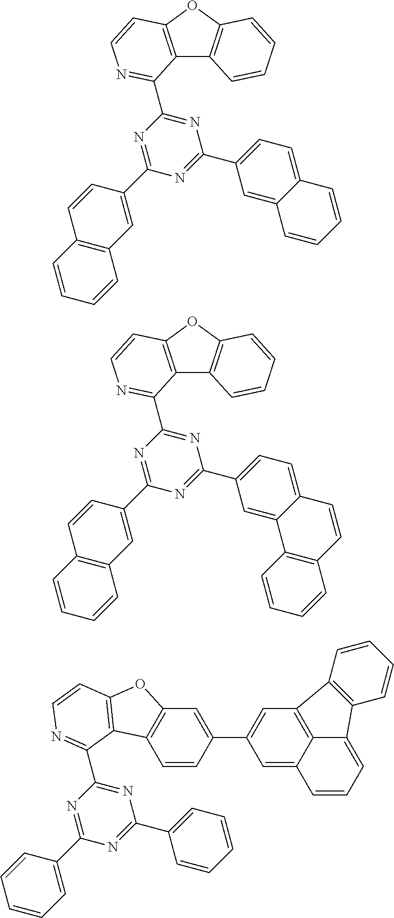 [SMILES: c1ccc2cc(-c3nc(-c4ccc5ccccc5c4)nc(-c4nccc5oc6ccccc6c45)n3)ccc2c1 ; c1ccc2cc(-c3nc(-c4ccc5ccc6ccccc6c5c4)nc(-c4nccc5oc6ccccc6c45)n3)ccc2c1 ; c1ccc(-c2nc(-c3ccccc3)nc(-c3nccc4oc5cc(-c6cc7c8c(cccc8c6)-c6ccccc6-7)ccc5c34)n2)cc1]

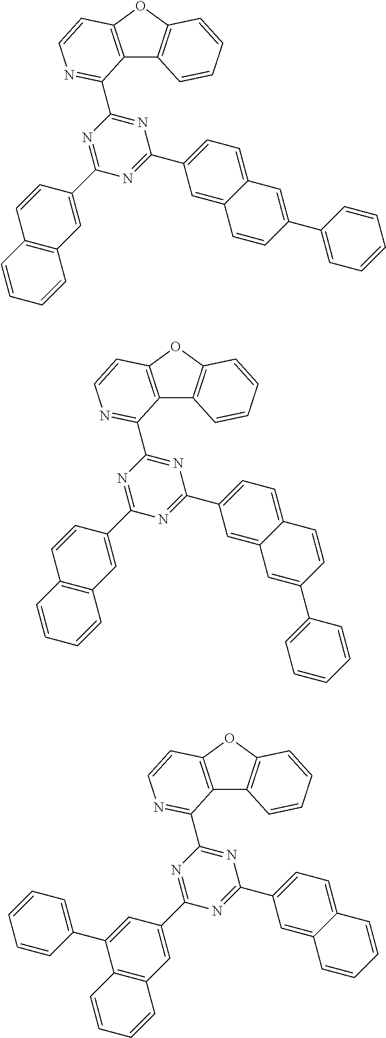 [SMILES: c1ccc(-c2ccc3cc(-c4nc(-c5ccc6ccccc6c5)nc(-c5nccc6oc7ccccc7c56)n4)ccc3c2)cc1 ; c1ccc(-c2ccc3ccc(-c4nc(-c5ccc6ccccc6c5)nc(-c5nccc6oc7ccccc7c56)n4)cc3c2)cc1 ; c1ccc(-c2cc(-c3nc(-c4ccc5ccccc5c4)nc(-c4nccc5oc6ccccc6c45)n3)cc3ccccc23)cc1]

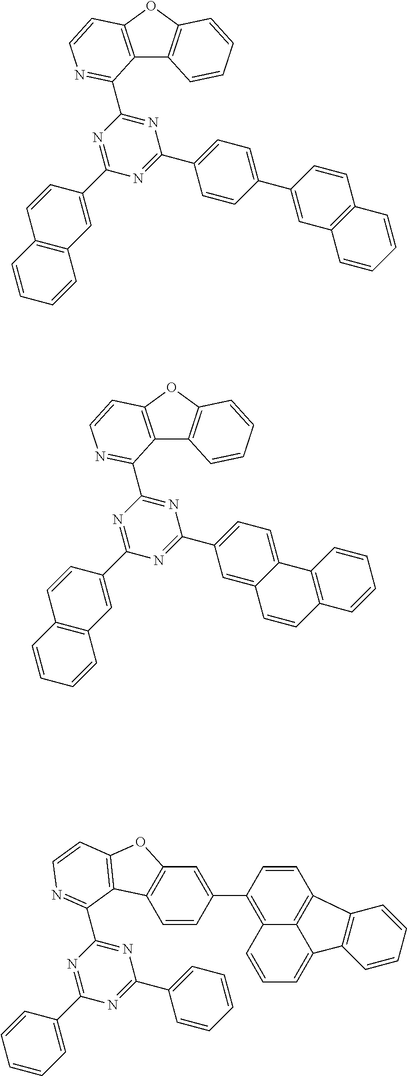 [SMILES: c1ccc2cc(-c3ccc(-c4nc(-c5ccc6ccccc6c5)nc(-c5nccc6oc7ccccc7c56)n4)cc3)ccc2c1 ; c1ccc2cc(-c3nc(-c4ccc5c(ccc6ccccc65)c4)nc(-c4nccc5oc6ccccc6c45)n3)ccc2c1 ; c1ccc(-c2nc(-c3ccccc3)nc(-c3nccc4oc5cc(-c6ccc7c8c(cccc68)-c6ccccc6-7)ccc5c34)n2)cc1]

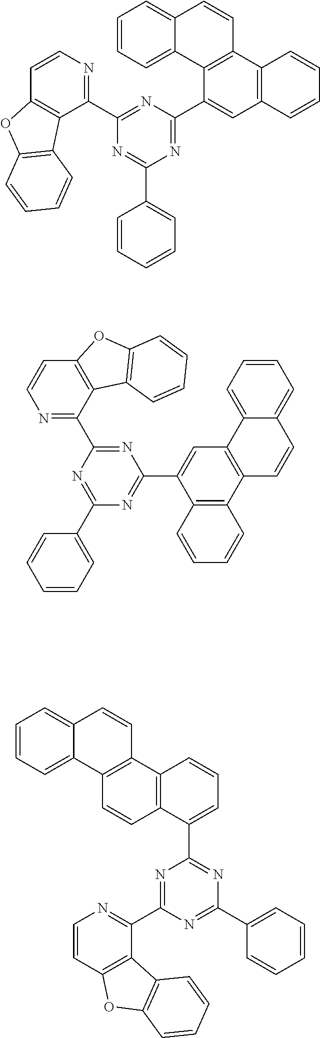 [SMILES: c1ccc(-c2nc(-c3nccc4oc5ccccc5c34)nc(-c3cc4ccccc4c4ccc5ccccc5c34)n2)cc1 ; c1ccc(-c2nc(-c3cc4c5ccccc5ccc4c4ccccc34)nc(-c3nccc4oc5ccccc5c34)n2)cc1 ; c1ccc(-c2nc(-c3cccc4c3ccc3c5ccccc5ccc43)nc(-c3nccc4oc5ccccc5c34)n2)cc1]

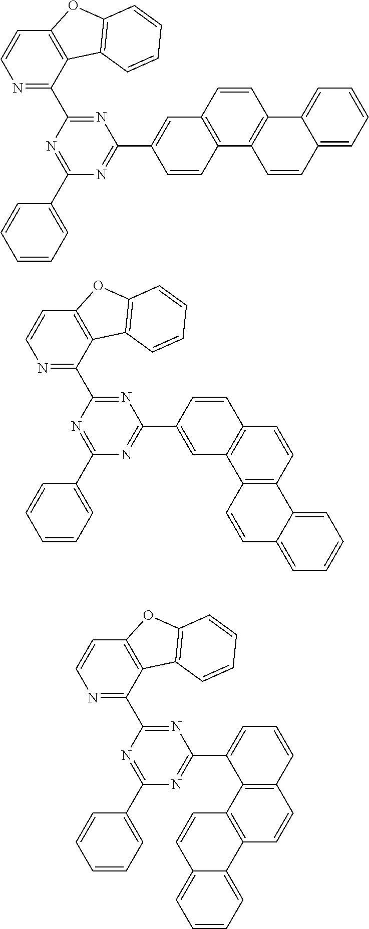 [SMILES: c1ccc(-c2nc(-c3ccc4c(ccc5c6ccccc6ccc45)c3)nc(-c3nccc4oc5ccccc5c34)n2)cc1 ; c1ccc(-c2nc(-c3ccc4ccc5c6ccccc6ccc5c4c3)nc(-c3nccc4oc5ccccc5c34)n2)cc1 ; c1ccc(-c2nc(-c3cccc4ccc5c6ccccc6ccc5c34)nc(-c3nccc4oc5ccccc5c34)n2)cc1]

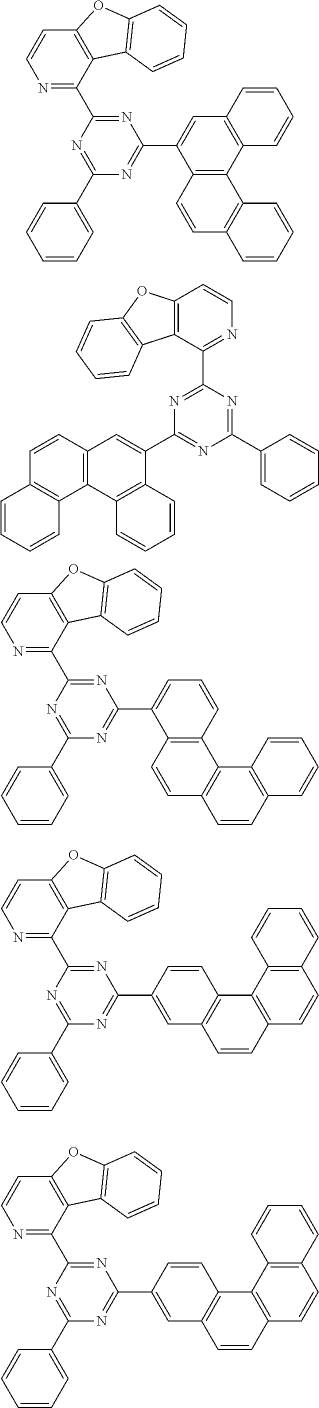

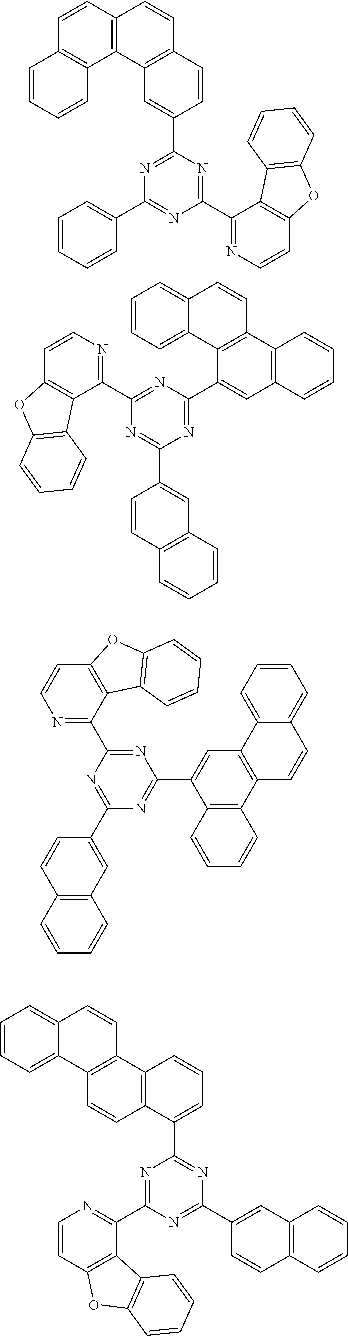

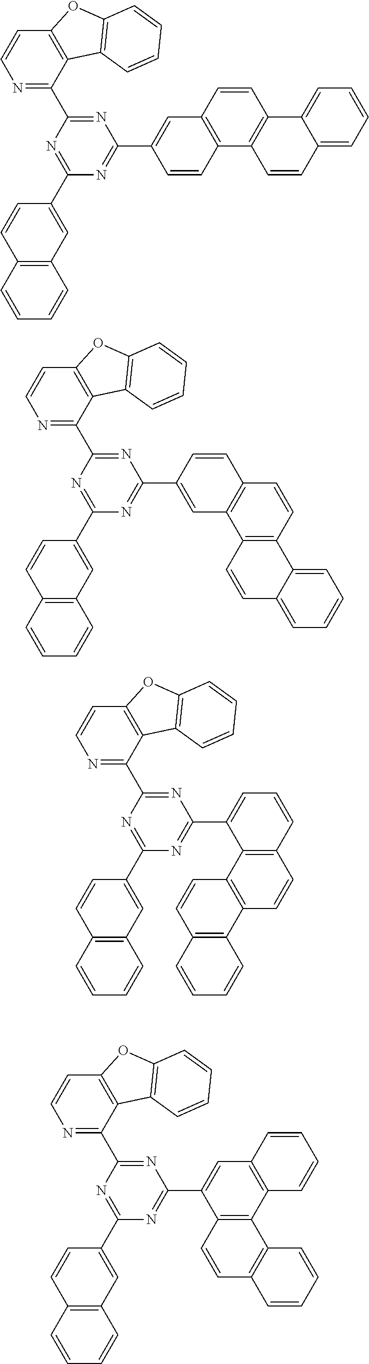 [SMILES: c1ccc2cc(-c3nc(-c4ccc5c(ccc6c7ccccc7ccc56)c4)nc(-c4nccc5oc6ccccc6c45)n3)ccc2c1 ; c1ccc2cc(-c3nc(-c4ccc5ccc6c7ccccc7ccc6c5c4)nc(-c4nccc5oc6ccccc6c45)n3)ccc2c1 ; c1ccc2cc(-c3nc(-c4cccc5ccc6c7ccccc7ccc6c45)nc(-c4nccc5oc6ccccc6c45)n3)ccc2c1 ; c1ccc2cc(-c3nc(-c4cc5ccccc5c5c4ccc4ccccc45)nc(-c4nccc5oc6ccccc6c45)n3)ccc2c1]

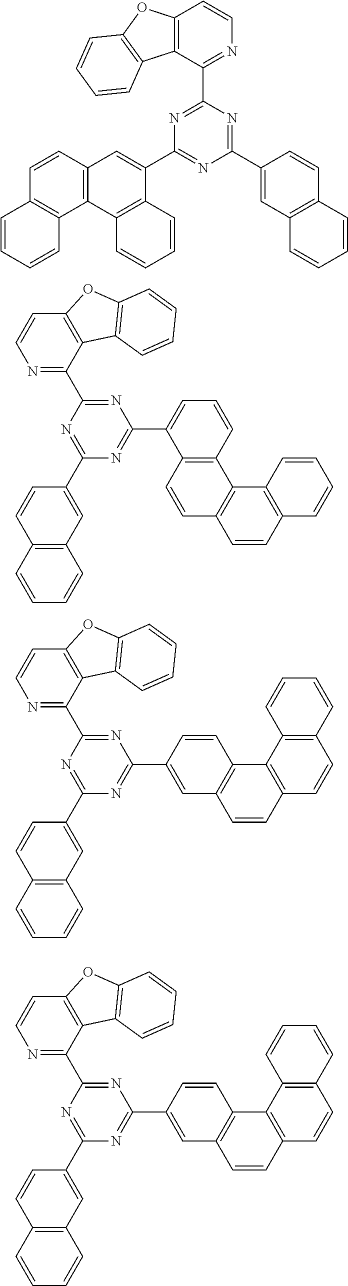

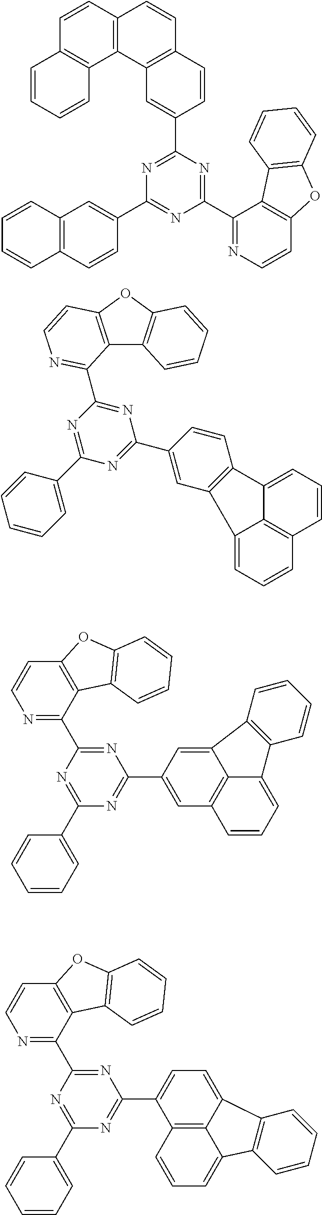

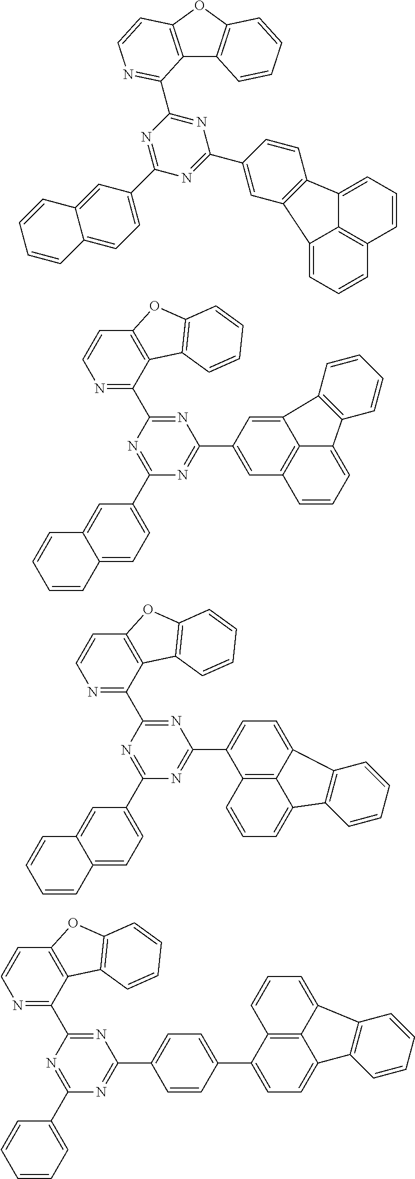

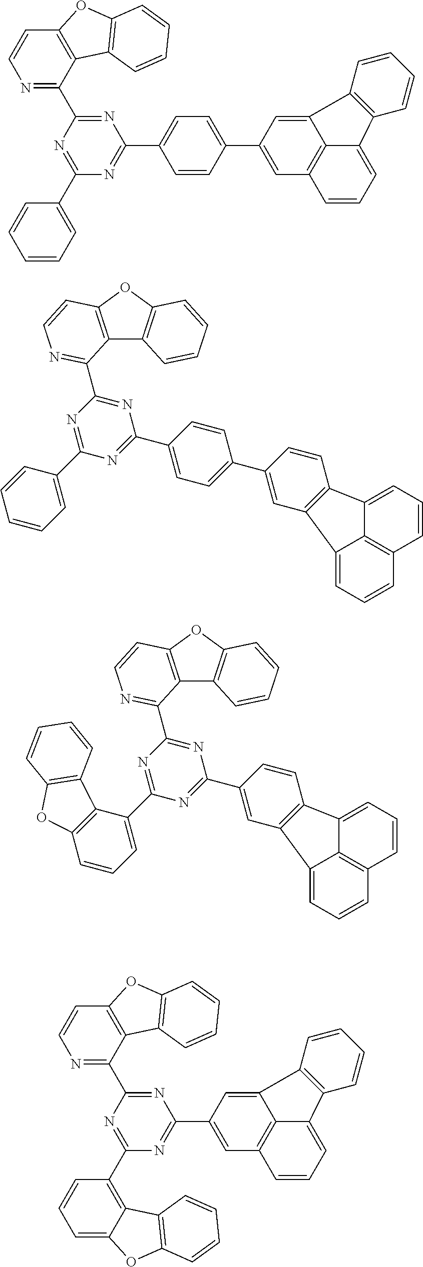

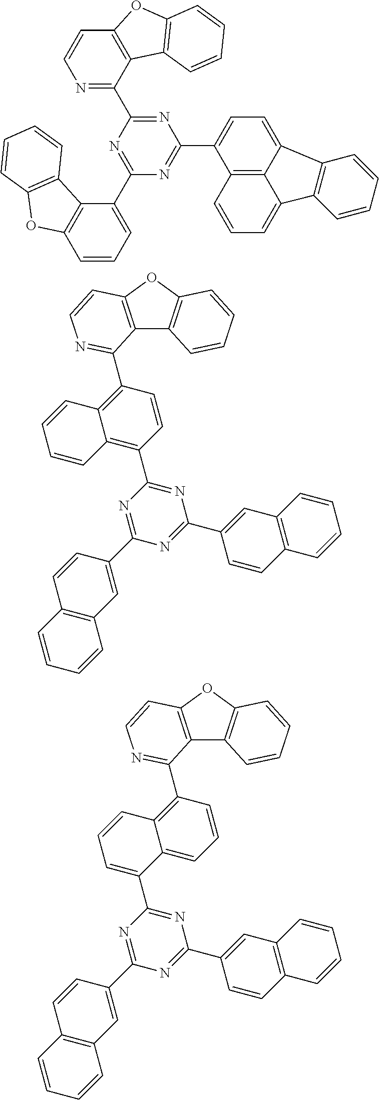

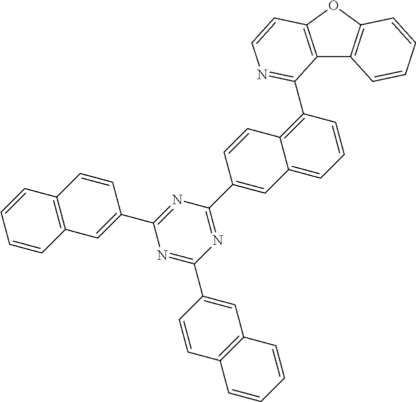 [SMILES: c1ccc2cc(-c3nc(-c4ccc5ccccc5c4)nc(-c4ccc5c(-c6nccc7oc8ccccc8c67)cccc5c4)n3)ccc2c1]

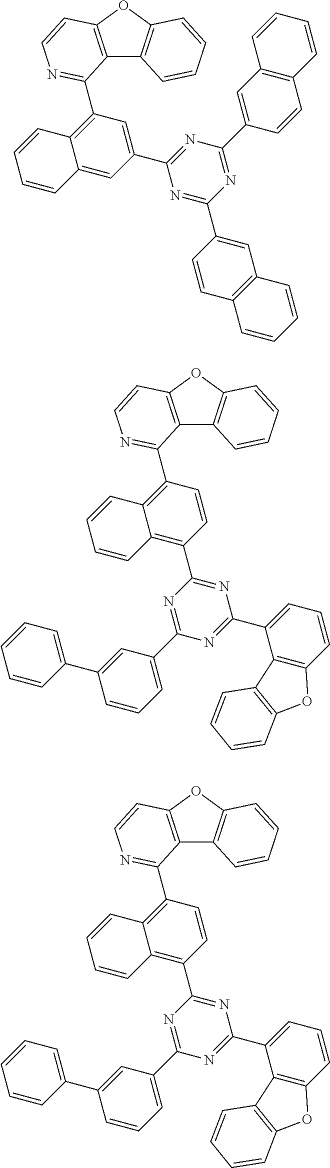 [SMILES: c1ccc2cc(-c3nc(-c4ccc5ccccc5c4)nc(-c4cc(-c5nccc6oc7ccccc7c56)c5ccccc5c4)n3)ccc2c1 ; c1ccc(-c2cccc(-c3nc(-c4ccc(-c5nccc6oc7ccccc7c56)c5ccccc45)nc(-c4cccc5oc6ccccc6c45)n3)c2)cc1 ; c1ccc(-c2cccc(-c3nc(-c4ccc(-c5nccc6oc7ccccc7c56)c5ccccc45)nc(-c4cccc5oc6ccccc6c45)n3)c2)cc1]

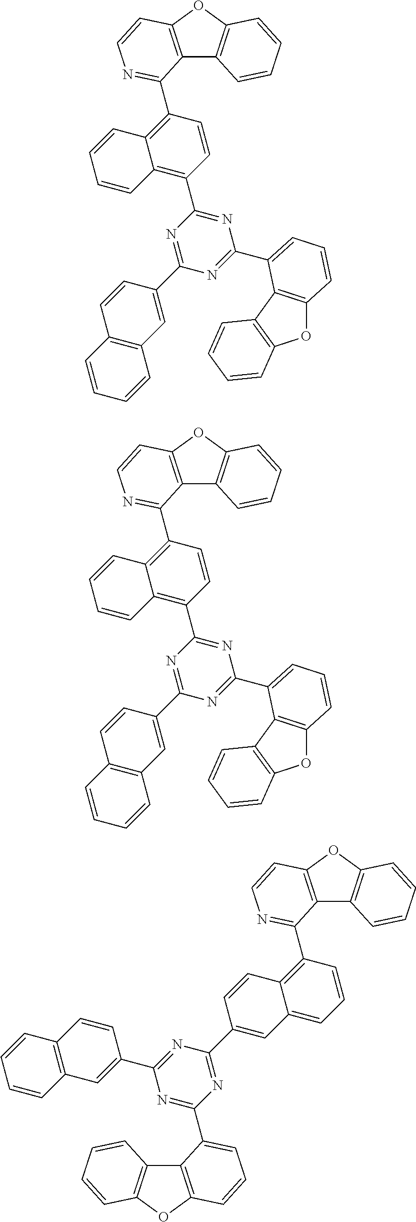

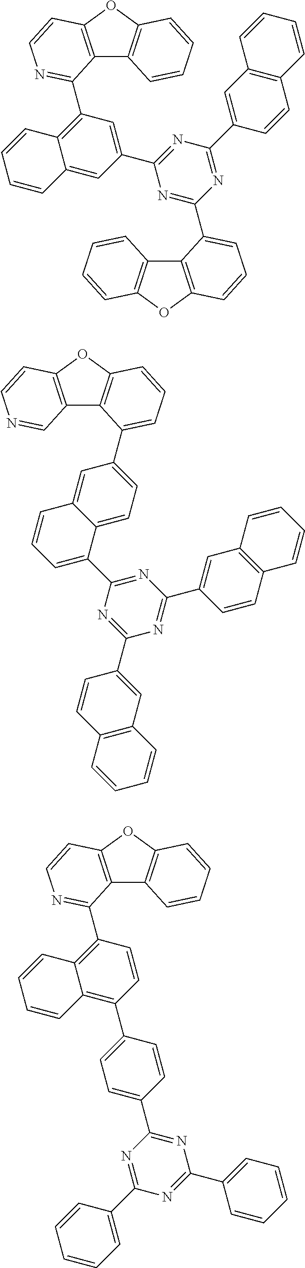

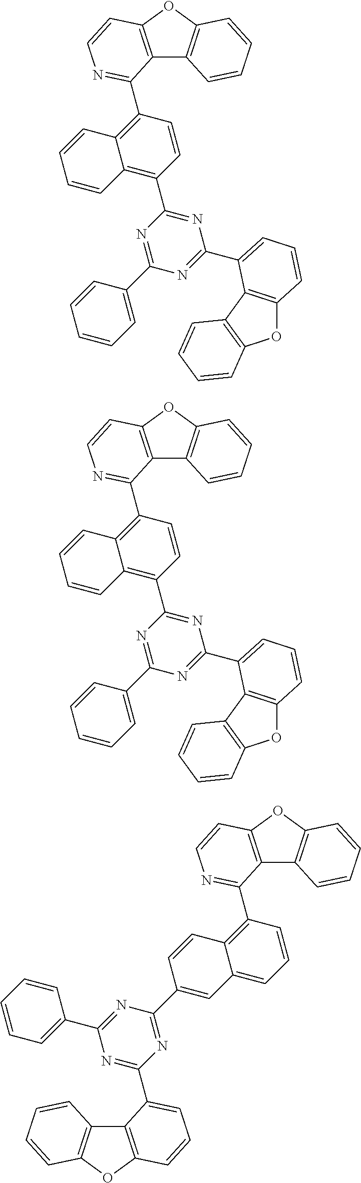

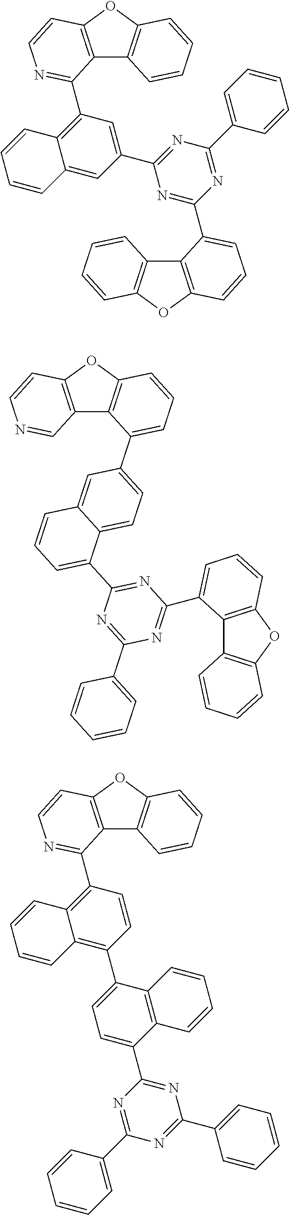

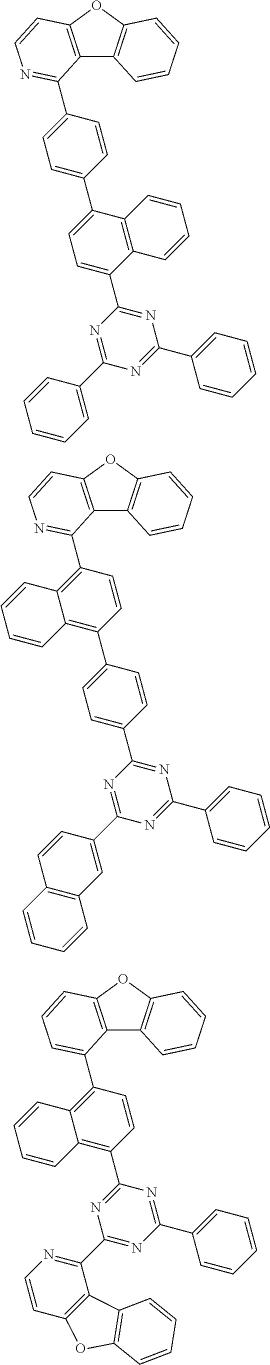

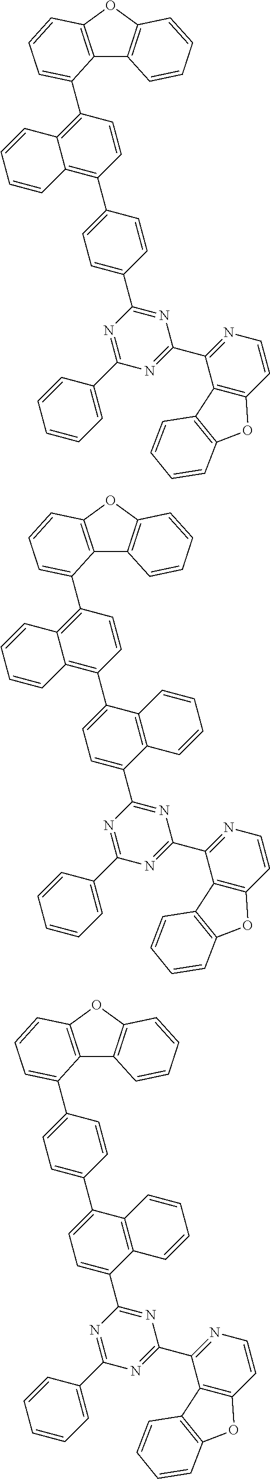

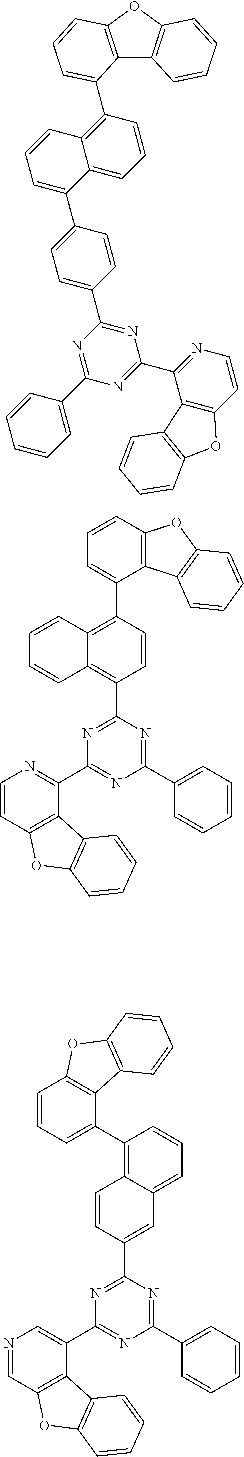

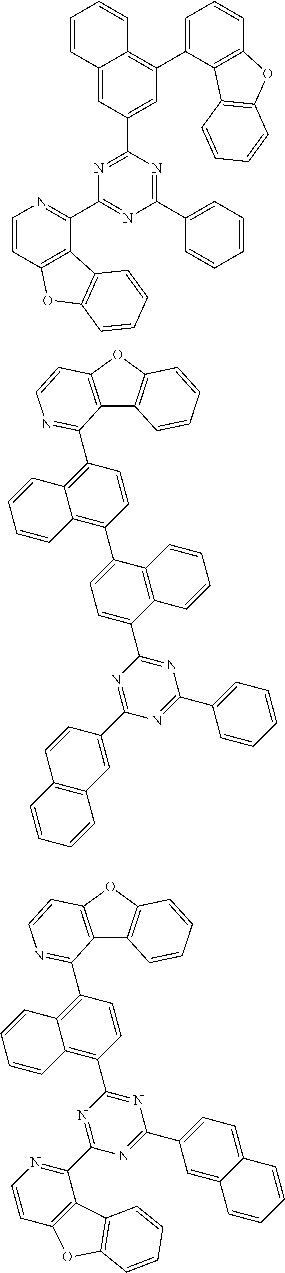

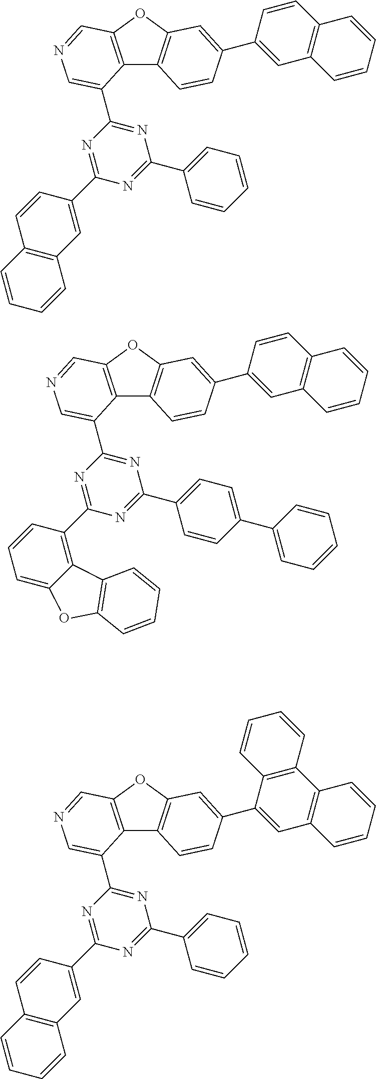 [SMILES: c1ccc(-c2nc(-c3ccc4ccccc4c3)nc(-c3cncc4oc5cc(-c6ccc7ccccc7c6)ccc5c34)n2)cc1 ; c1ccc(-c2ccc(-c3nc(-c4cccc5oc6ccccc6c45)nc(-c4cncc5oc6cc(-c7ccc8ccccc8c7)ccc6c45)n3)cc2)cc1 ; c1ccc(-c2nc(-c3ccc4ccccc4c3)nc(-c3cncc4oc5cc(-c6cc7ccccc7c7ccccc67)ccc5c34)n2)cc1]

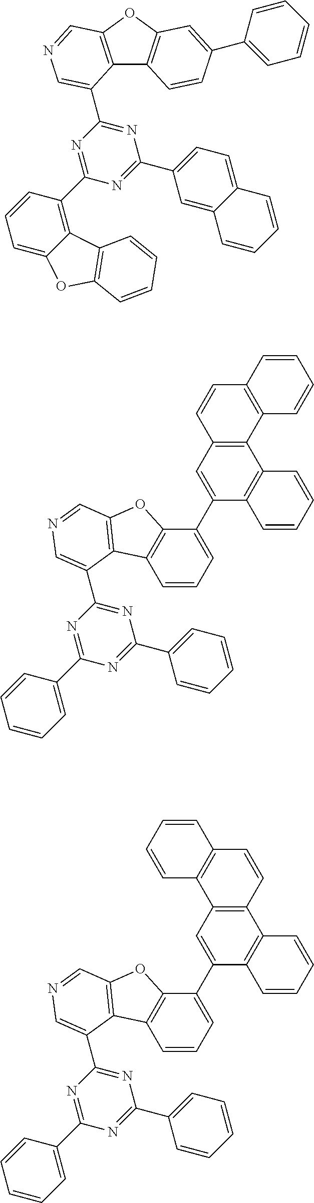 [SMILES: c1ccc(-c2ccc3c(c2)oc2cncc(-c4nc(-c5ccc6ccccc6c5)nc(-c5cccc6oc7ccccc7c56)n4)c23)cc1 ; c1ccc(-c2nc(-c3ccccc3)nc(-c3cncc4oc5c(-c6cc7ccc8ccccc8c7c7ccccc67)cccc5c34)n2)cc1 ; c1ccc(-c2nc(-c3ccccc3)nc(-c3cncc4oc5c(-c6cc7c8ccccc8ccc7c7ccccc67)cccc5c34)n2)cc1]

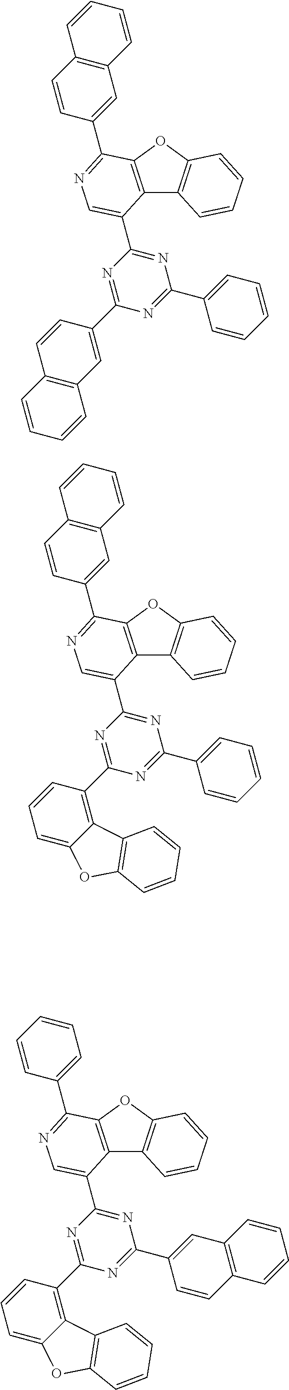

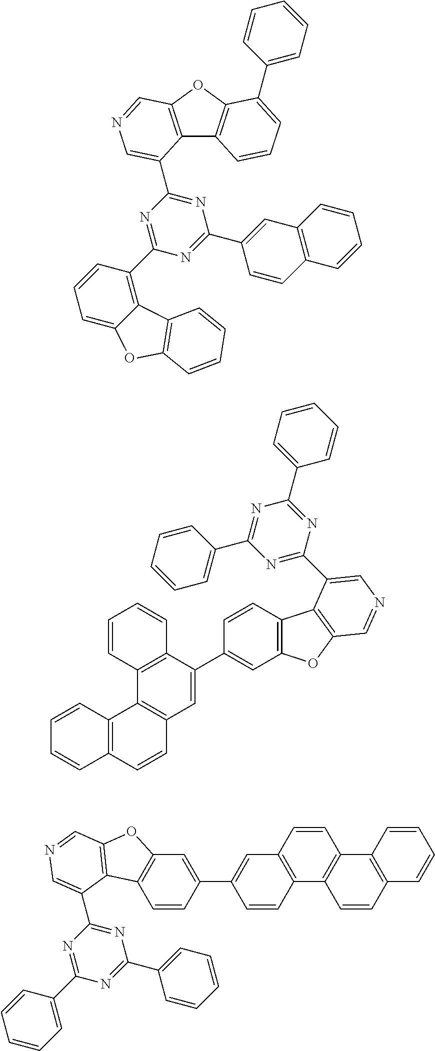 [SMILES: c1ccc(-c2cccc3c2oc2cncc(-c4nc(-c5ccc6ccccc6c5)nc(-c5cccc6oc7ccccc7c56)n4)c23)cc1 ; c1ccc(-c2nc(-c3ccccc3)nc(-c3cncc4oc5cc(-c6cc7ccc8ccccc8c7c7ccccc67)ccc5c34)n2)cc1 ; c1ccc(-c2nc(-c3ccccc3)nc(-c3cncc4oc5cc(-c6ccc7c(ccc8c9ccccc9ccc78)c6)ccc5c34)n2)cc1]

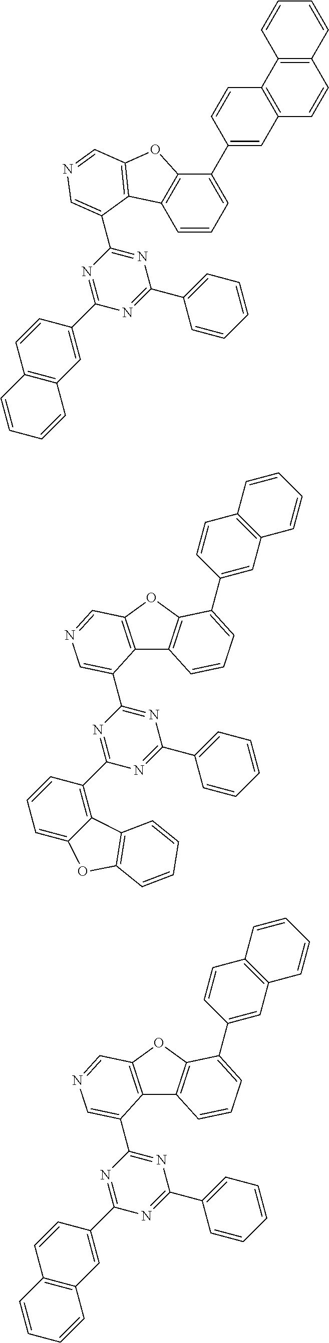 [SMILES: c1ccc(-c2nc(-c3ccc4ccccc4c3)nc(-c3cncc4oc5c(-c6ccc7c(ccc8ccccc87)c6)cccc5c34)n2)cc1 ; c1ccc(-c2nc(-c3cccc4oc5ccccc5c34)nc(-c3cncc4oc5c(-c6ccc7ccccc7c6)cccc5c34)n2)cc1 ; c1ccc(-c2nc(-c3ccc4ccccc4c3)nc(-c3cncc4oc5c(-c6ccc7ccccc7c6)cccc5c34)n2)cc1]

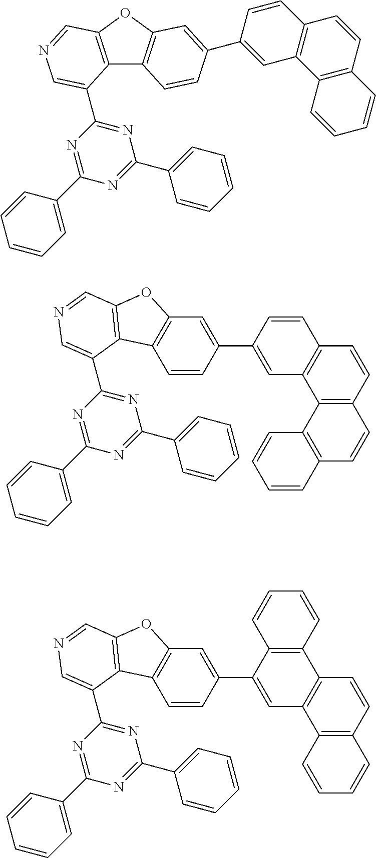 [SMILES: c1ccc(-c2nc(-c3ccccc3)nc(-c3cncc4oc5cc(-c6ccc7ccc8ccccc8c7c6)ccc5c34)n2)cc1 ; c1ccc(-c2nc(-c3ccccc3)nc(-c3cncc4oc5cc(-c6ccc7ccc8ccc9ccccc9c8c7c6)ccc5c34)n2)cc1 ; c1ccc(-c2nc(-c3ccccc3)nc(-c3cncc4oc5cc(-c6cc7c8ccccc8ccc7c7ccccc67)ccc5c34)n2)cc1]

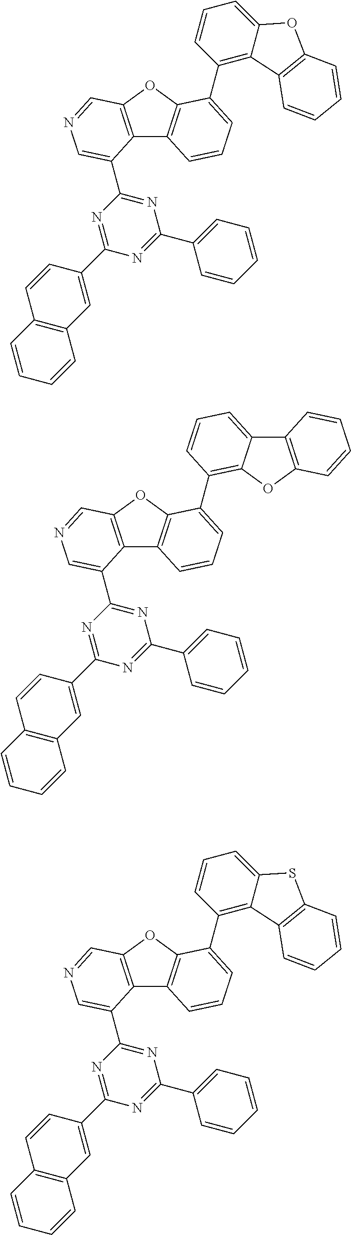 [SMILES: c1ccc(-c2nc(-c3ccc4ccccc4c3)nc(-c3cncc4oc5c(-c6cccc7oc8ccccc8c67)cccc5c34)n2)cc1 ; c1ccc(-c2nc(-c3ccc4ccccc4c3)nc(-c3cncc4oc5c(-c6cccc7c6oc6ccccc67)cccc5c34)n2)cc1 ; c1ccc(-c2nc(-c3ccc4ccccc4c3)nc(-c3cncc4oc5c(-c6cccc7sc8ccccc8c67)cccc5c34)n2)cc1]

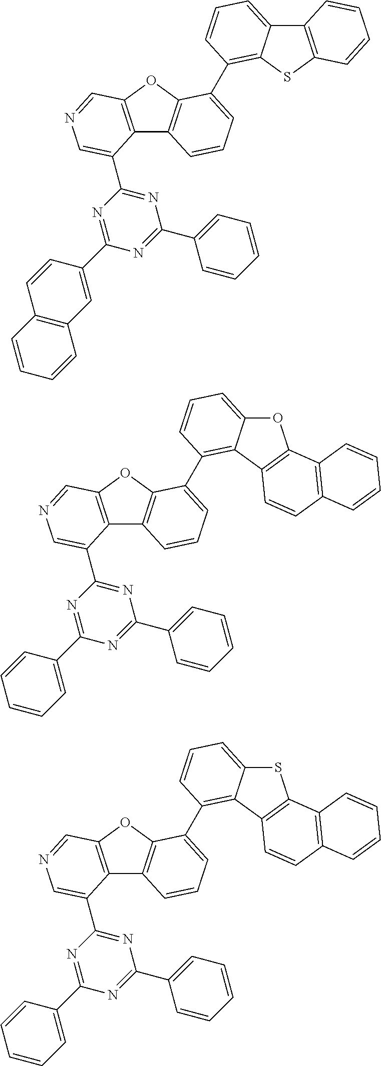 [SMILES: c1ccc(-c2nc(-c3ccc4ccccc4c3)nc(-c3cncc4oc5c(-c6cccc7c6sc6ccccc67)cccc5c34)n2)cc1 ; c1ccc(-c2nc(-c3ccccc3)nc(-c3cncc4oc5c(-c6cccc7oc8c9ccccc9ccc8c67)cccc5c34)n2)cc1 ; c1ccc(-c2nc(-c3ccccc3)nc(-c3cncc4oc5c(-c6cccc7sc8c9ccccc9ccc8c67)cccc5c34)n2)cc1]

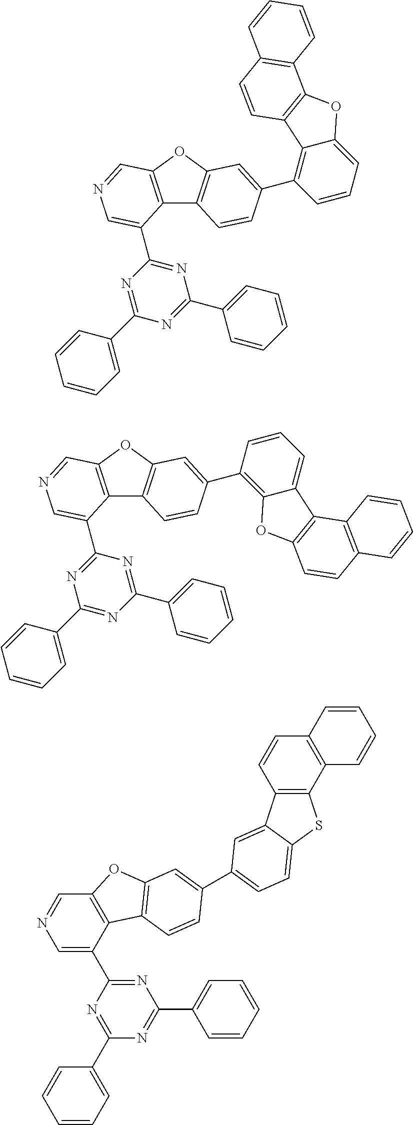 [SMILES: c1ccc(-c2nc(-c3ccccc3)nc(-c3cncc4oc5cc(-c6cccc7oc8c9ccccc9ccc8c67)ccc5c34)n2)cc1 ; c1ccc(-c2nc(-c3ccccc3)nc(-c3cncc4oc5cc(-c6cccc7c6oc6ccc8ccccc8c67)ccc5c34)n2)cc1 ; c1ccc(-c2nc(-c3ccccc3)nc(-c3cncc4oc5cc(-c6ccc7sc8c9ccccc9ccc8c7c6)ccc5c34)n2)cc1]

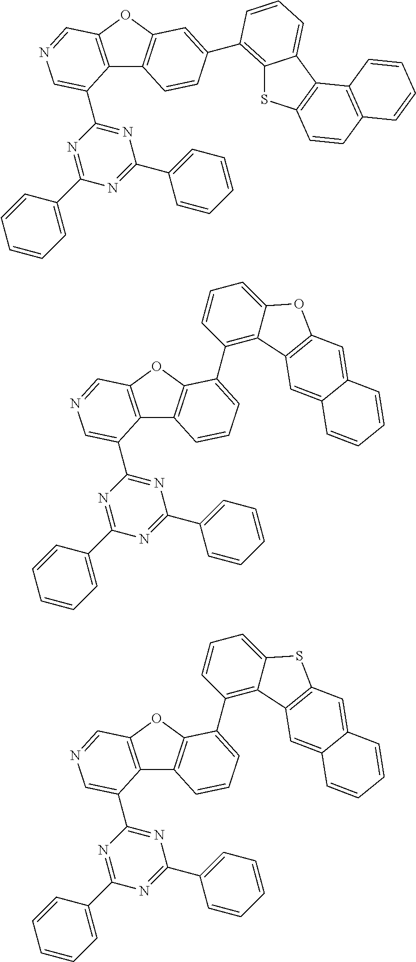 [SMILES: c1ccc(-c2nc(-c3ccccc3)nc(-c3cncc4oc5cc(-c6cccc7c6sc6ccc8ccccc8c67)ccc5c34)n2)cc1 ; c1ccc(-c2nc(-c3ccccc3)nc(-c3cncc4oc5c(-c6cccc7oc8cc9ccccc9cc8c67)cccc5c34)n2)cc1 ; c1ccc(-c2nc(-c3ccccc3)nc(-c3cncc4oc5c(-c6cccc7sc8cc9ccccc9cc8c67)cccc5c34)n2)cc1]

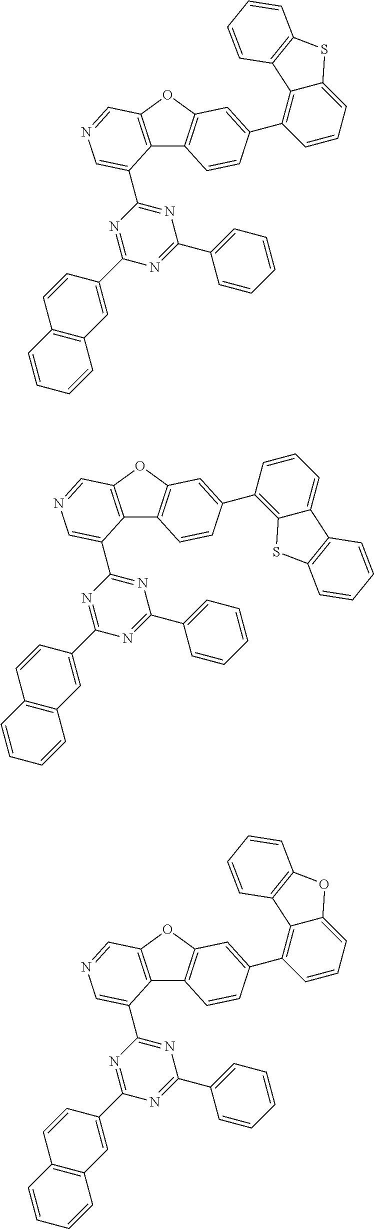 [SMILES: c1ccc(-c2nc(-c3ccc4ccccc4c3)nc(-c3cncc4oc5cc(-c6cccc7sc8ccccc8c67)ccc5c34)n2)cc1 ; c1ccc(-c2nc(-c3ccc4ccccc4c3)nc(-c3cncc4oc5cc(-c6cccc7c6sc6ccccc67)ccc5c34)n2)cc1 ; c1ccc(-c2nc(-c3ccc4ccccc4c3)nc(-c3cncc4oc5cc(-c6cccc7oc8ccccc8c67)ccc5c34)n2)cc1]

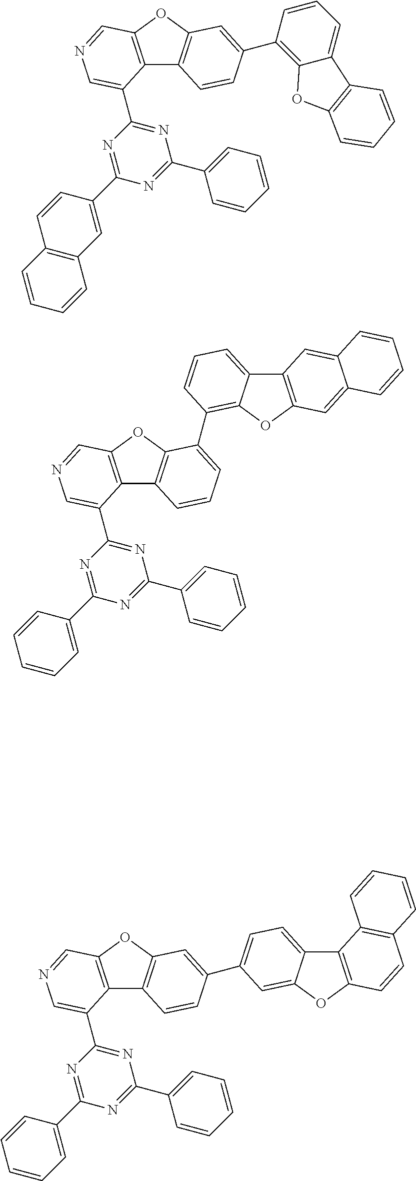 [SMILES: c1ccc(-c2nc(-c3ccc4ccccc4c3)nc(-c3cncc4oc5cc(-c6cccc7c6oc6ccccc67)ccc5c34)n2)cc1 ; c1ccc(-c2nc(-c3ccccc3)nc(-c3cncc4oc5c(-c6cccc7c6oc6cc8ccccc8cc67)cccc5c34)n2)cc1 ; c1ccc(-c2nc(-c3ccccc3)nc(-c3cncc4oc5cc(-c6ccc7c(c6)oc6ccc8ccccc8c67)ccc5c34)n2)cc1]

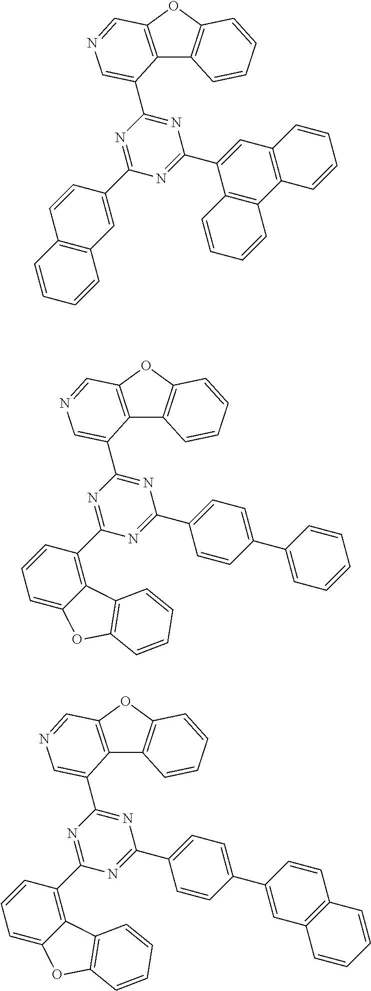 [SMILES: c1ccc2cc(-c3nc(-c4cc5ccccc5c5ccccc45)nc(-c4cncc5oc6ccccc6c45)n3)ccc2c1 ; c1ccc(-c2ccc(-c3nc(-c4cccc5oc6ccccc6c45)nc(-c4cncc5oc6ccccc6c45)n3)cc2)cc1 ; c1ccc2cc(-c3ccc(-c4nc(-c5cccc6oc7ccccc7c56)nc(-c5cncc6oc7ccccc7c56)n4)cc3)ccc2c1]

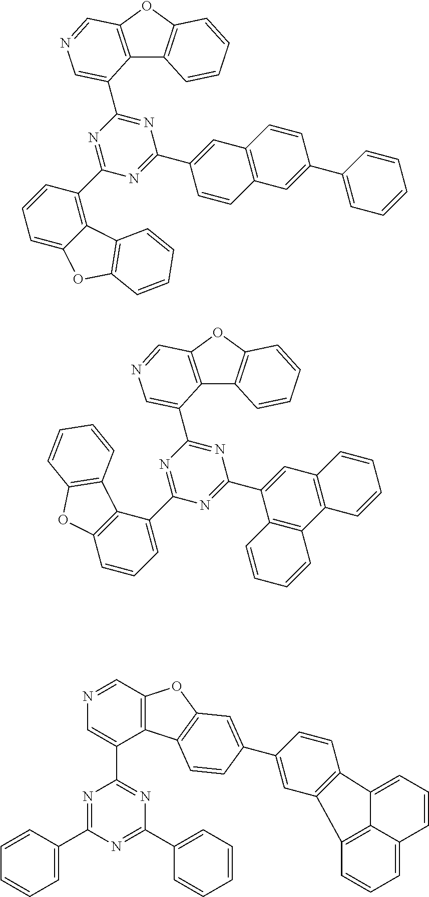 [SMILES: c1ccc(-c2ccc3cc(-c4nc(-c5cccc6oc7ccccc7c56)nc(-c5cncc6oc7ccccc7c56)n4)ccc3c2)cc1 ; c1ccc2c(c1)cc(-c1nc(-c3cccc4oc5ccccc5c34)nc(-c3cncc4oc5ccccc5c34)n1)c1ccccc12 ; c1ccc(-c2nc(-c3ccccc3)nc(-c3cncc4oc5cc(-c6ccc7c(c6)-c6cccc8cccc-7c68)ccc5c34)n2)cc1]

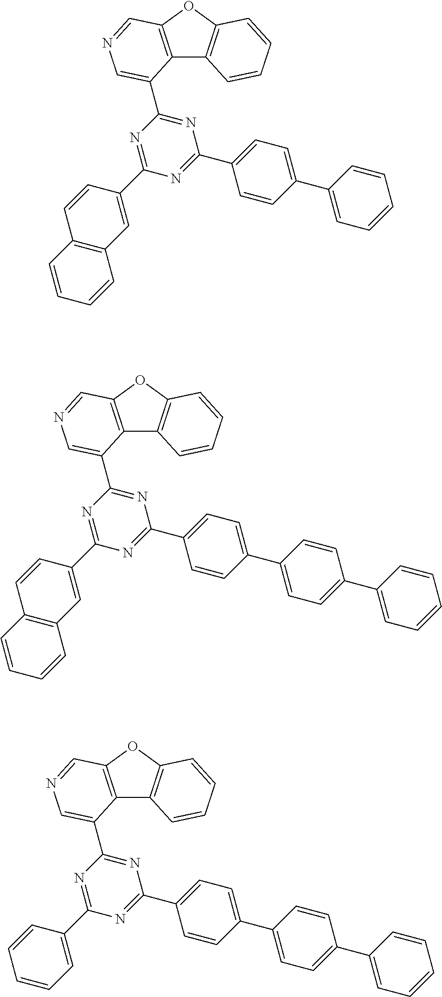 [SMILES: c1ccc(-c2ccc(-c3nc(-c4ccc5ccccc5c4)nc(-c4cncc5oc6ccccc6c45)n3)cc2)cc1 ; c1ccc(-c2ccc(-c3ccc(-c4nc(-c5ccc6ccccc6c5)nc(-c5cncc6oc7ccccc7c56)n4)cc3)cc2)cc1 ; c1ccc(-c2ccc(-c3ccc(-c4nc(-c5ccccc5)nc(-c5cncc6oc7ccccc7c56)n4)cc3)cc2)cc1]

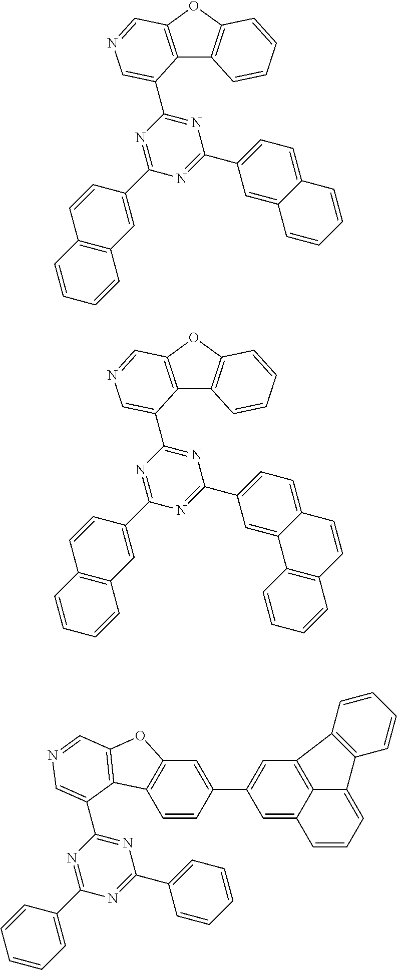 [SMILES: c1ccc2cc(-c3nc(-c4ccc5ccccc5c4)nc(-c4cncc5oc6ccccc6c45)n3)ccc2c1 ; c1ccc2cc(-c3nc(-c4ccc5ccc6ccccc6c5c4)nc(-c4cncc5oc6ccccc6c45)n3)ccc2c1 ; c1ccc(-c2nc(-c3ccccc3)nc(-c3cncc4oc5cc(-c6cc7c8c(cccc8c6)-c6ccccc6-7)ccc5c34)n2)cc1]

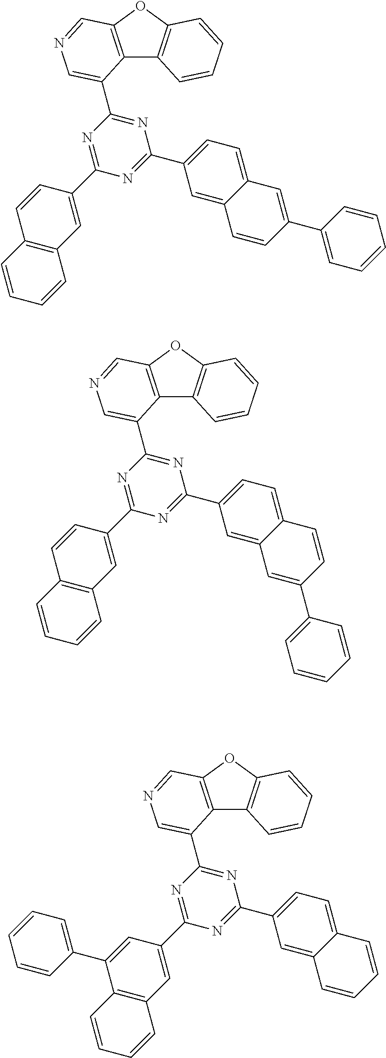 [SMILES: c1ccc(-c2ccc3cc(-c4nc(-c5ccc6ccccc6c5)nc(-c5cncc6oc7ccccc7c56)n4)ccc3c2)cc1 ; c1ccc(-c2ccc3ccc(-c4nc(-c5ccc6ccccc6c5)nc(-c5cncc6oc7ccccc7c56)n4)cc3c2)cc1 ; c1ccc(-c2cc(-c3nc(-c4ccc5ccccc5c4)nc(-c4cncc5oc6ccccc6c45)n3)cc3ccccc23)cc1]

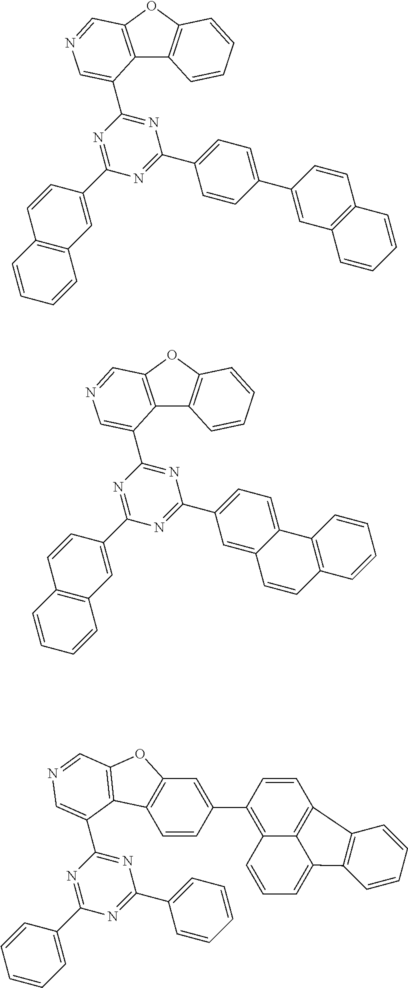 [SMILES: c1ccc2cc(-c3ccc(-c4nc(-c5ccc6ccccc6c5)nc(-c5cncc6oc7ccccc7c56)n4)cc3)ccc2c1 ; c1ccc2cc(-c3nc(-c4ccc5c(ccc6ccccc65)c4)nc(-c4cncc5oc6ccccc6c45)n3)ccc2c1 ; c1ccc(-c2nc(-c3ccccc3)nc(-c3cncc4oc5cc(-c6ccc7c8c(cccc68)-c6ccccc6-7)ccc5c34)n2)cc1]

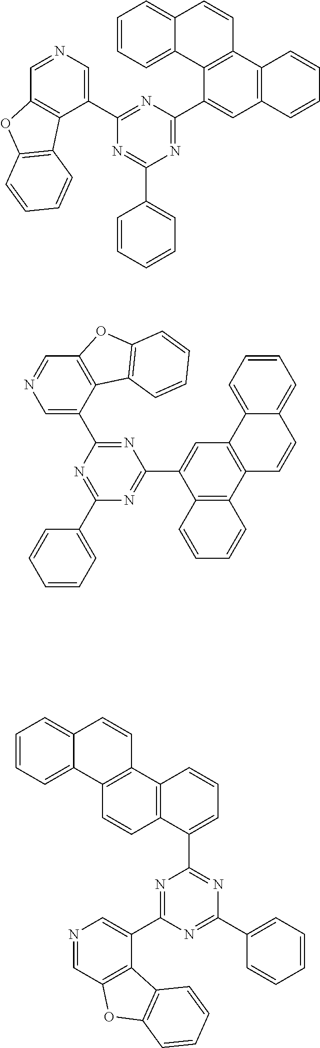 [SMILES: c1ccc(-c2nc(-c3cncc4oc5ccccc5c34)nc(-c3cc4ccccc4c4ccc5ccccc5c34)n2)cc1 ; c1ccc(-c2nc(-c3cc4c5ccccc5ccc4c4ccccc34)nc(-c3cncc4oc5ccccc5c34)n2)cc1 ; c1ccc(-c2nc(-c3cccc4c3ccc3c5ccccc5ccc43)nc(-c3cncc4oc5ccccc5c34)n2)cc1]

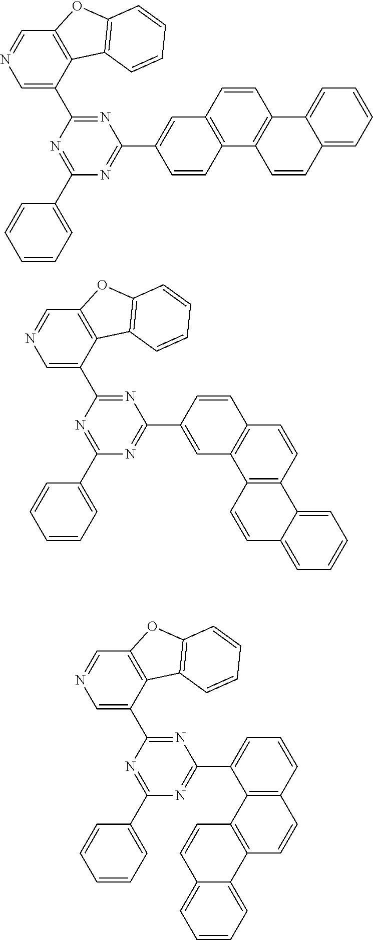 [SMILES: c1ccc(-c2nc(-c3ccc4c(ccc5c6ccccc6ccc45)c3)nc(-c3cncc4oc5ccccc5c34)n2)cc1 ; c1ccc(-c2nc(-c3ccc4ccc5c6ccccc6ccc5c4c3)nc(-c3cncc4oc5ccccc5c34)n2)cc1 ; c1ccc(-c2nc(-c3cccc4ccc5c6ccccc6ccc5c34)nc(-c3cncc4oc5ccccc5c34)n2)cc1]

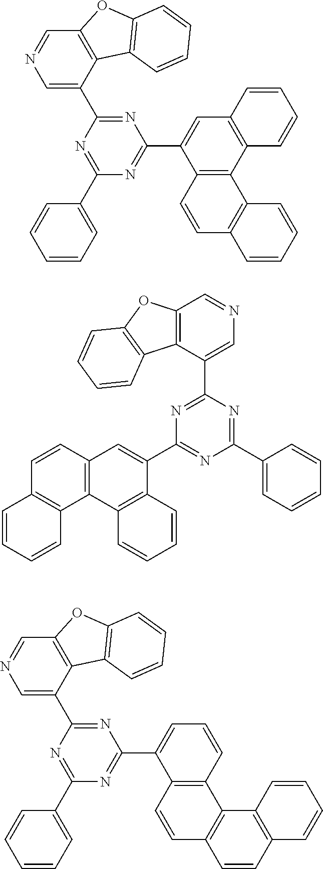 [SMILES: c1ccc(-c2nc(-c3cc4ccccc4c4c3ccc3ccccc34)nc(-c3cncc4oc5ccccc5c34)n2)cc1 ; c1ccc(-c2nc(-c3cc4ccc5ccccc5c4c4ccccc34)nc(-c3cncc4oc5ccccc5c34)n2)cc1 ; c1ccc(-c2nc(-c3cccc4c3ccc3ccc5ccccc5c34)nc(-c3cncc4oc5ccccc5c34)n2)cc1]

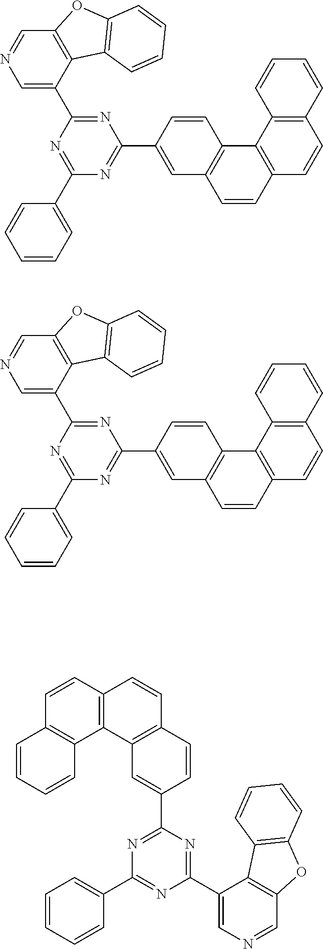 [SMILES: c1ccc(-c2nc(-c3ccc4c(ccc5ccc6ccccc6c54)c3)nc(-c3cncc4oc5ccccc5c34)n2)cc1 ; c1ccc(-c2nc(-c3ccc4c(ccc5ccc6ccccc6c54)c3)nc(-c3cncc4oc5ccccc5c34)n2)cc1 ; c1ccc(-c2nc(-c3ccc4ccc5ccc6ccccc6c5c4c3)nc(-c3cncc4oc5ccccc5c34)n2)cc1]

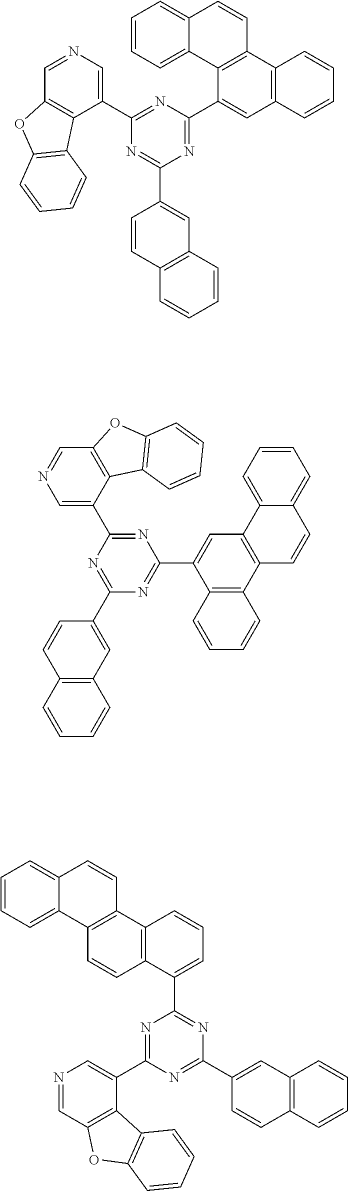 [SMILES: c1ccc2cc(-c3nc(-c4cncc5oc6ccccc6c45)nc(-c4cc5ccccc5c5ccc6ccccc6c45)n3)ccc2c1 ; c1ccc2cc(-c3nc(-c4cc5c6ccccc6ccc5c5ccccc45)nc(-c4cncc5oc6ccccc6c45)n3)ccc2c1 ; c1ccc2cc(-c3nc(-c4cccc5c4ccc4c6ccccc6ccc54)nc(-c4cncc5oc6ccccc6c45)n3)ccc2c1]

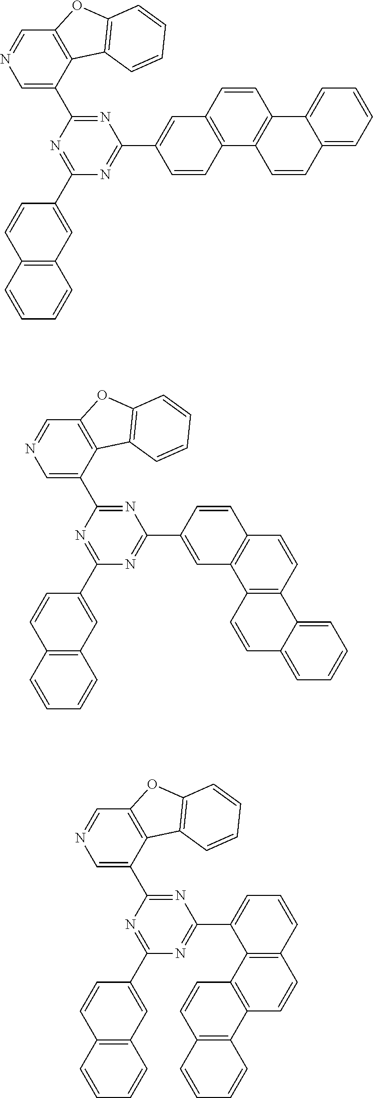 [SMILES: c1ccc2cc(-c3nc(-c4ccc5c(ccc6c7ccccc7ccc56)c4)nc(-c4cncc5oc6ccccc6c45)n3)ccc2c1 ; c1ccc2cc(-c3nc(-c4ccc5ccc6c7ccccc7ccc6c5c4)nc(-c4cncc5oc6ccccc6c45)n3)ccc2c1 ; c1ccc2cc(-c3nc(-c4cccc5ccc6c7ccccc7ccc6c45)nc(-c4cncc5oc6ccccc6c45)n3)ccc2c1]

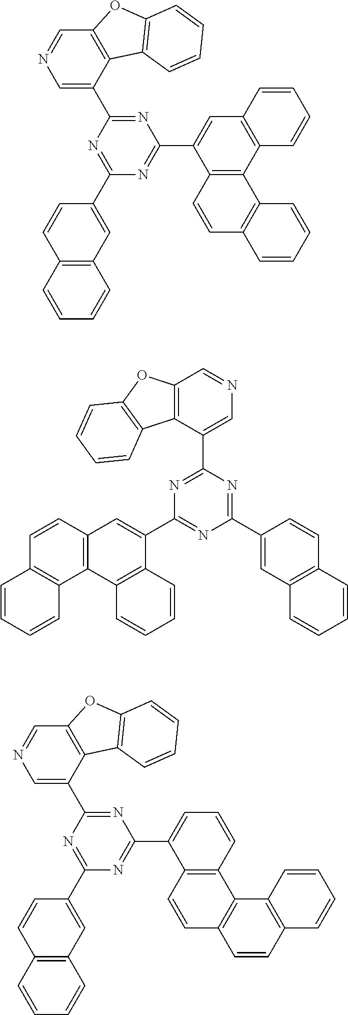 [SMILES: c1ccc2cc(-c3nc(-c4cc5ccccc5c5c4ccc4ccccc45)nc(-c4cncc5oc6ccccc6c45)n3)ccc2c1 ; c1ccc2cc(-c3nc(-c4cc5ccc6ccccc6c5c5ccccc45)nc(-c4cncc5oc6ccccc6c45)n3)ccc2c1 ; c1ccc2cc(-c3nc(-c4cccc5c4ccc4ccc6ccccc6c45)nc(-c4cncc5oc6ccccc6c45)n3)ccc2c1]

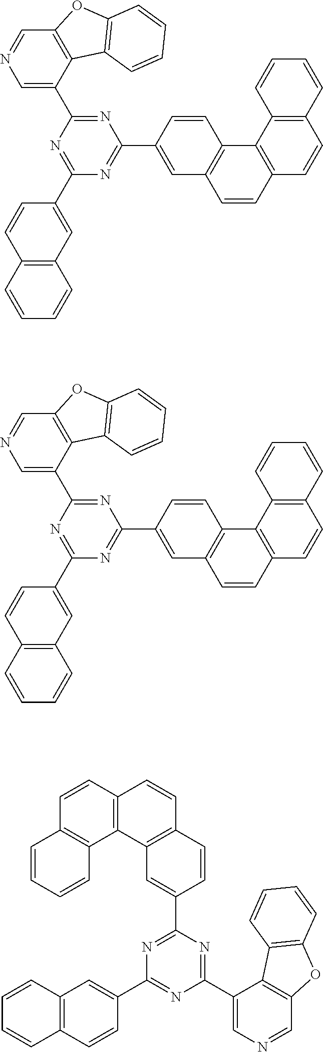 [SMILES: c1ccc2cc(-c3nc(-c4ccc5c(ccc6ccc7ccccc7c65)c4)nc(-c4cncc5oc6ccccc6c45)n3)ccc2c1 ; c1ccc2cc(-c3nc(-c4ccc5c(ccc6ccc7ccccc7c65)c4)nc(-c4cncc5oc6ccccc6c45)n3)ccc2c1 ; c1ccc2cc(-c3nc(-c4ccc5ccc6ccc7ccccc7c6c5c4)nc(-c4cncc5oc6ccccc6c45)n3)ccc2c1]

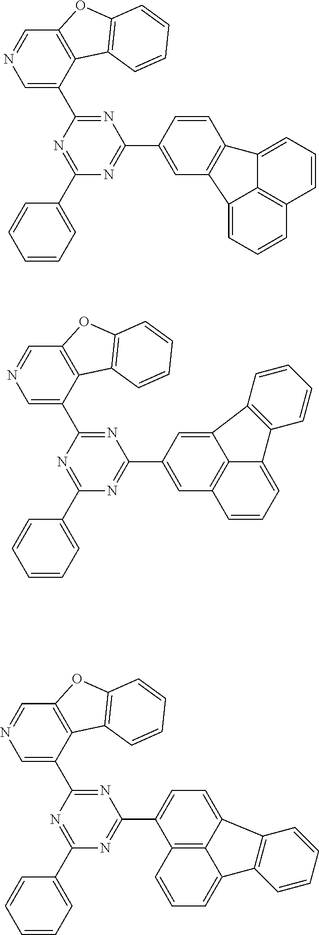 [SMILES: c1ccc(-c2nc(-c3ccc4c(c3)-c3cccc5cccc-4c35)nc(-c3cncc4oc5ccccc5c34)n2)cc1 ; c1ccc(-c2nc(-c3cc4c5c(cccc5c3)-c3ccccc3-4)nc(-c3cncc4oc5ccccc5c34)n2)cc1 ; c1ccc(-c2nc(-c3ccc4c5c(cccc35)-c3ccccc3-4)nc(-c3cncc4oc5ccccc5c34)n2)cc1]

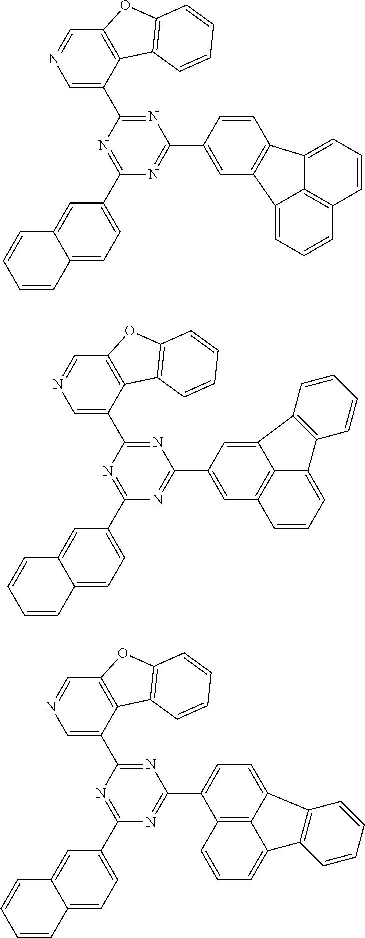 [SMILES: c1ccc2cc(-c3nc(-c4ccc5c(c4)-c4cccc6cccc-5c46)nc(-c4cncc5oc6ccccc6c45)n3)ccc2c1 ; c1ccc2c(c1)-c1cccc3cc(-c4nc(-c5ccc6ccccc6c5)nc(-c5cncc6oc7ccccc7c56)n4)cc-2c13 ; c1ccc2c(c1)-c1cccc3c(-c4nc(-c5ccc6ccccc6c5)nc(-c5cncc6oc7ccccc7c56)n4)ccc-2c13]

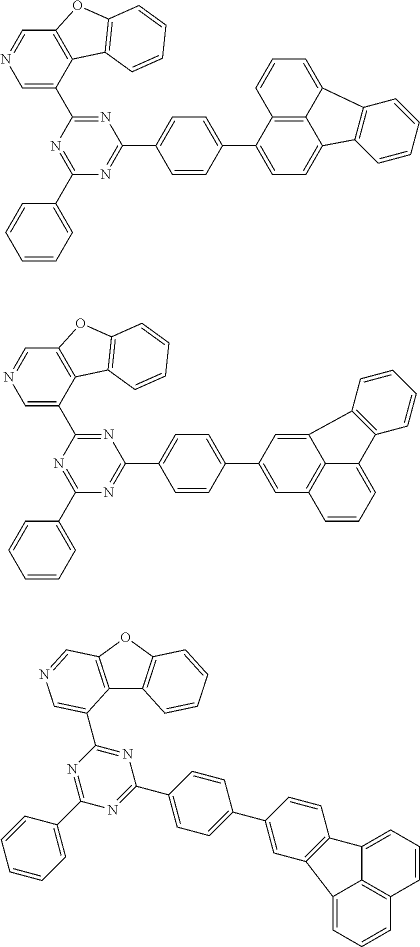 [SMILES: c1ccc(-c2nc(-c3ccc(-c4ccc5c6c(cccc46)-c4ccccc4-5)cc3)nc(-c3cncc4oc5ccccc5c34)n2)cc1 ; c1ccc(-c2nc(-c3ccc(-c4cc5c6c(cccc6c4)-c4ccccc4-5)cc3)nc(-c3cncc4oc5ccccc5c34)n2)cc1 ; c1ccc(-c2nc(-c3ccc(-c4ccc5c(c4)-c4cccc6cccc-5c46)cc3)nc(-c3cncc4oc5ccccc5c34)n2)cc1]

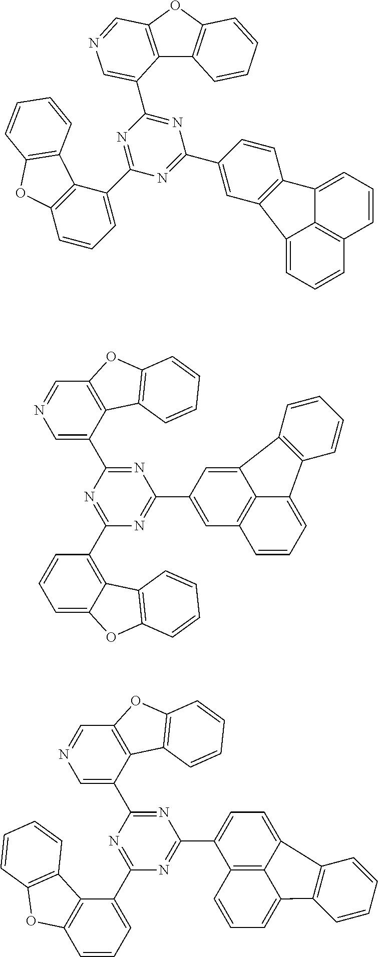 [SMILES: c1cc2c3c(cccc3c1)-c1cc(-c3nc(-c4cccc5oc6ccccc6c45)nc(-c4cncc5oc6ccccc6c45)n3)ccc1-2 ; c1ccc2c(c1)-c1cccc3cc(-c4nc(-c5cccc6oc7ccccc7c56)nc(-c5cncc6oc7ccccc7c56)n4)cc-2c13 ; c1ccc2c(c1)-c1cccc3c(-c4nc(-c5cccc6oc7ccccc7c56)nc(-c5cncc6oc7ccccc7c56)n4)ccc-2c13]

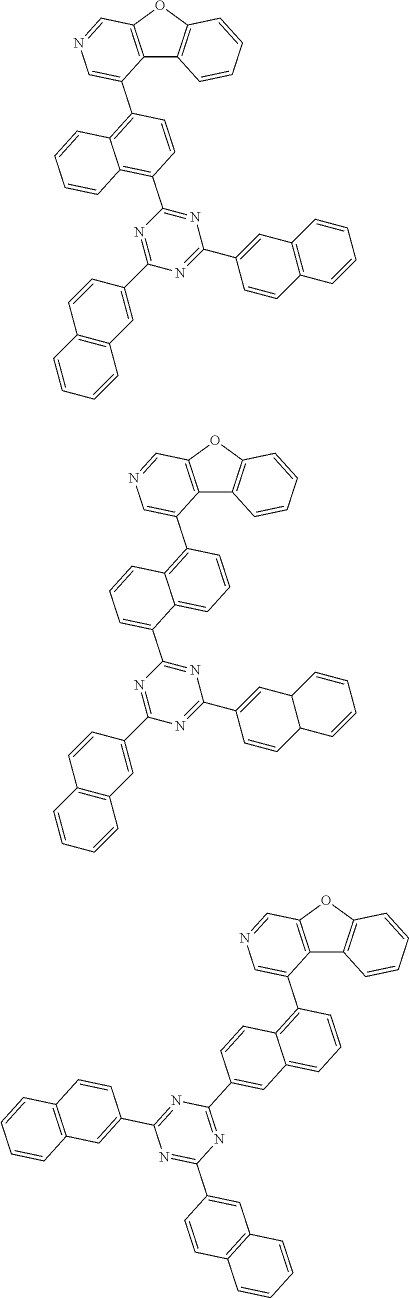 [SMILES: c1ccc2cc(-c3nc(-c4ccc5ccccc5c4)nc(-c4ccc(-c5cncc6oc7ccccc7c56)c5ccccc45)n3)ccc2c1 ; C1=CC2C=CC(c3nc(-c4ccc5ccccc5c4)nc(-c4cccc5c(-c6cncc7oc8ccccc8c67)cccc45)n3)=CC2C=C1 ; c1ccc2cc(-c3nc(-c4ccc5ccccc5c4)nc(-c4ccc5c(-c6cncc7oc8ccccc8c67)cccc5c4)n3)ccc2c1]

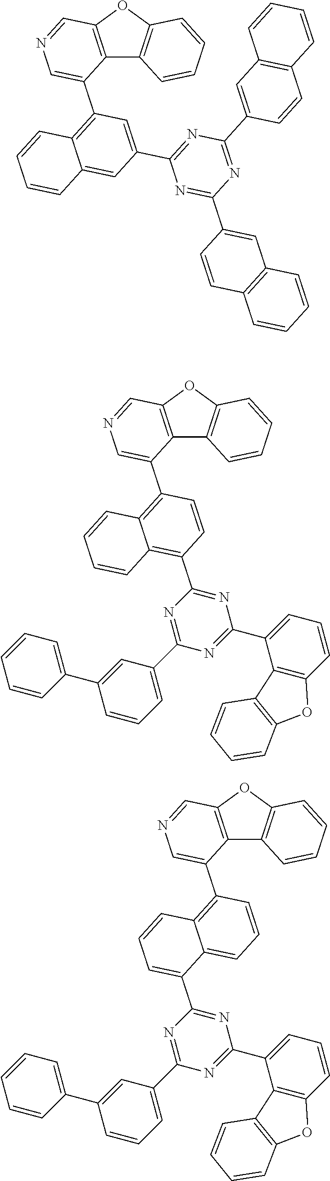

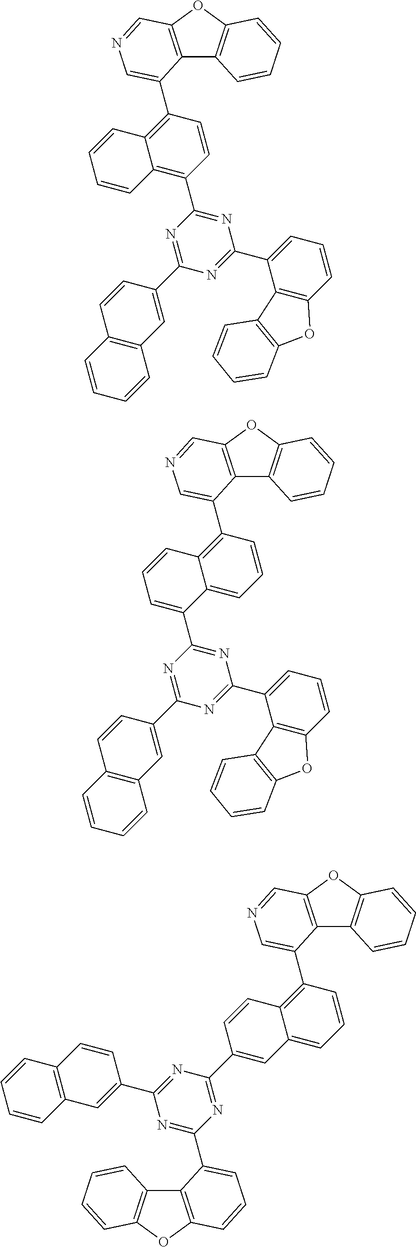

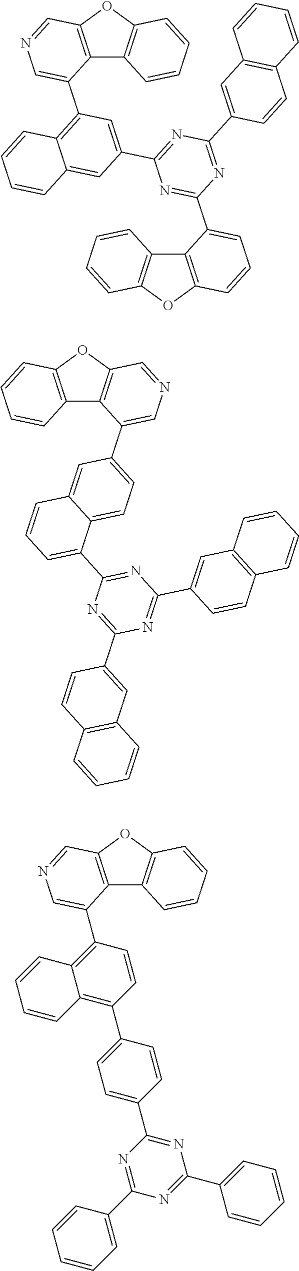 [SMILES: c1ccc2cc(-c3nc(-c4cc(-c5cncc6oc7ccccc7c56)c5ccccc5c4)nc(-c4cccc5oc6ccccc6c45)n3)ccc2c1 ; c1ccc2cc(-c3nc(-c4ccc5ccccc5c4)nc(-c4cccc5cc(-c6cncc7oc8ccccc8c67)ccc45)n3)ccc2c1 ; c1ccc(-c2nc(-c3ccccc3)nc(-c3ccc(-c4ccc(-c5cncc6oc7ccccc7c56)c5ccccc45)cc3)n2)cc1]

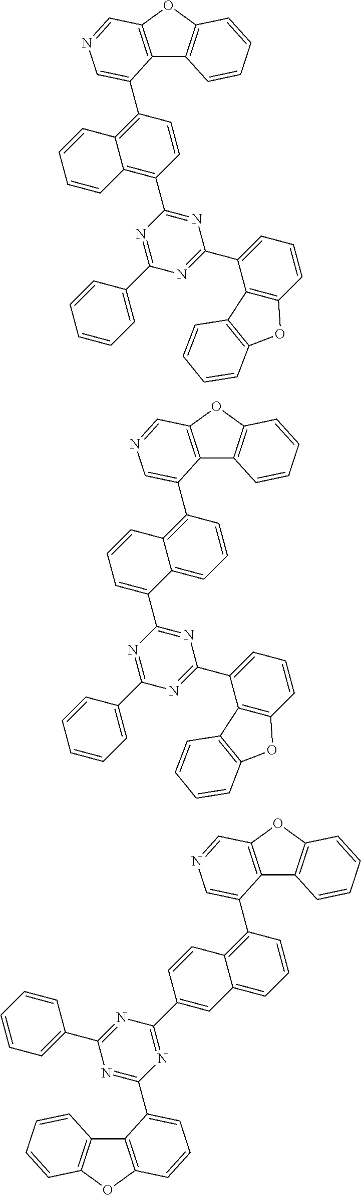

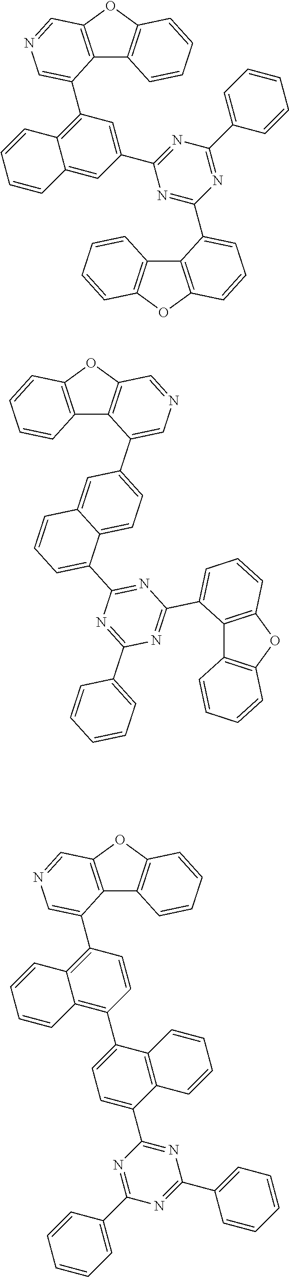 [SMILES: c1ccc(-c2nc(-c3cc(-c4cncc5oc6ccccc6c45)c4ccccc4c3)nc(-c3cccc4oc5ccccc5c34)n2)cc1 ; c1ccc(-c2nc(-c3cccc4cc(-c5cncc6oc7ccccc7c56)ccc34)nc(-c3cccc4oc5ccccc5c34)n2)cc1 ; c1ccc(-c2nc(-c3ccccc3)nc(-c3ccc(-c4ccc(-c5cncc6oc7ccccc7c56)c5ccccc45)c4ccccc34)n2)cc1]

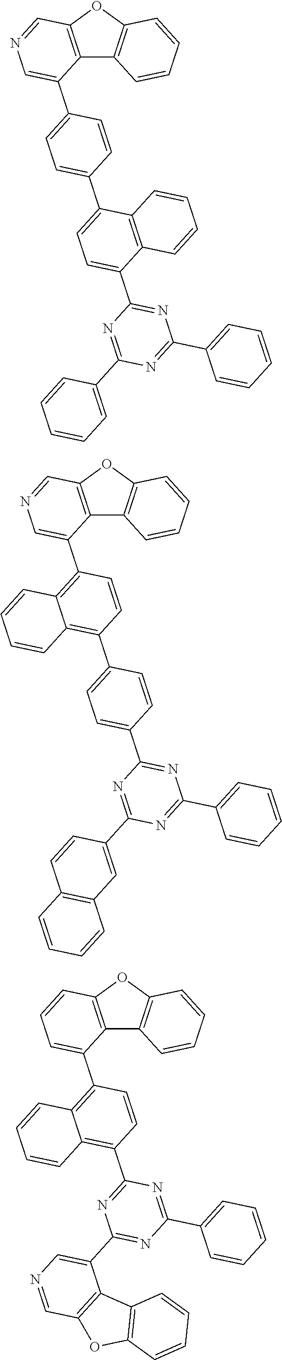

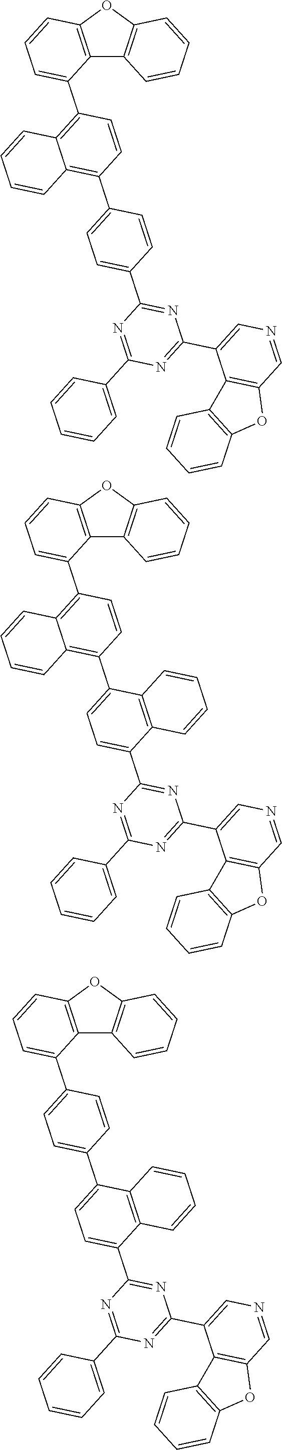

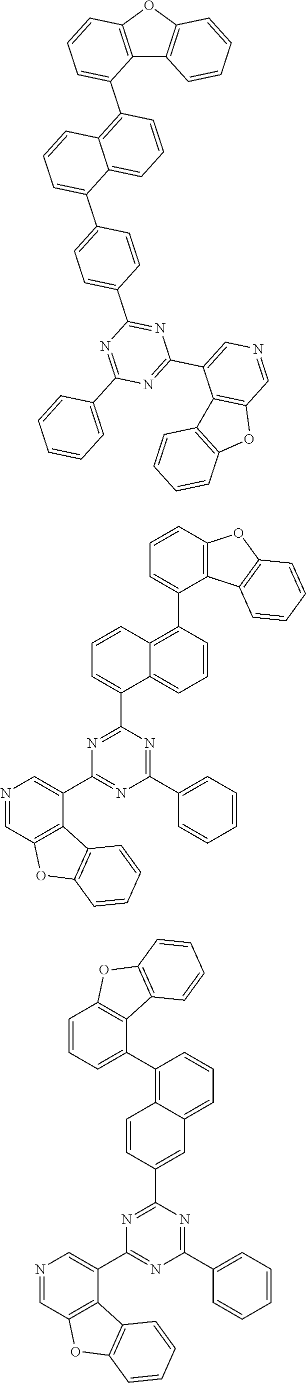

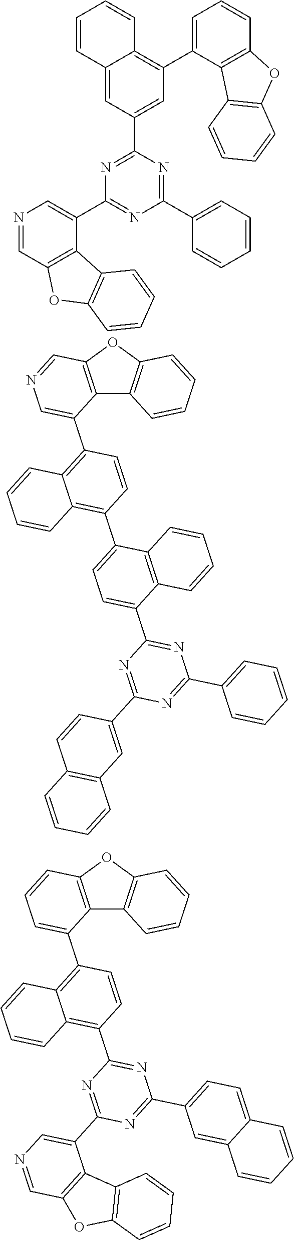

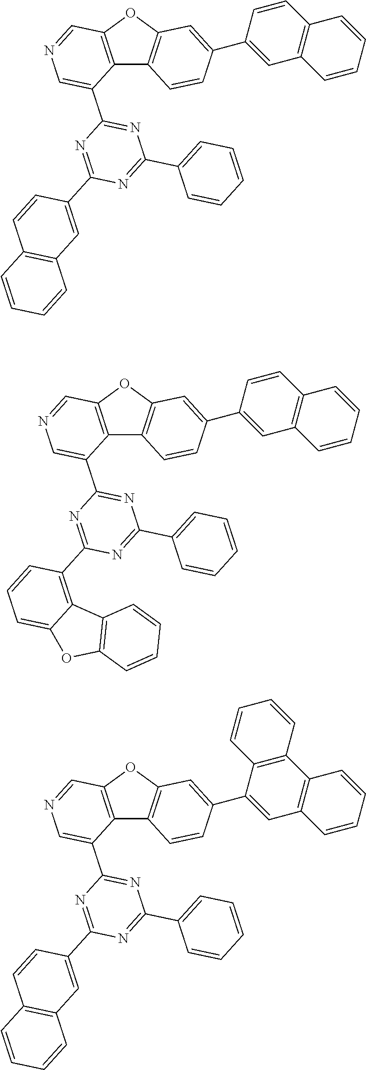 [SMILES: c1ccc(-c2nc(-c3ccc4ccccc4c3)nc(-c3cncc4oc5cc(-c6ccc7ccccc7c6)ccc5c34)n2)cc1 ; c1ccc(-c2nc(-c3cccc4oc5ccccc5c34)nc(-c3cncc4oc5cc(-c6ccc7ccccc7c6)ccc5c34)n2)cc1 ; c1ccc(-c2nc(-c3ccc4ccccc4c3)nc(-c3cncc4oc5cc(-c6cc7ccccc7c7ccccc67)ccc5c34)n2)cc1]

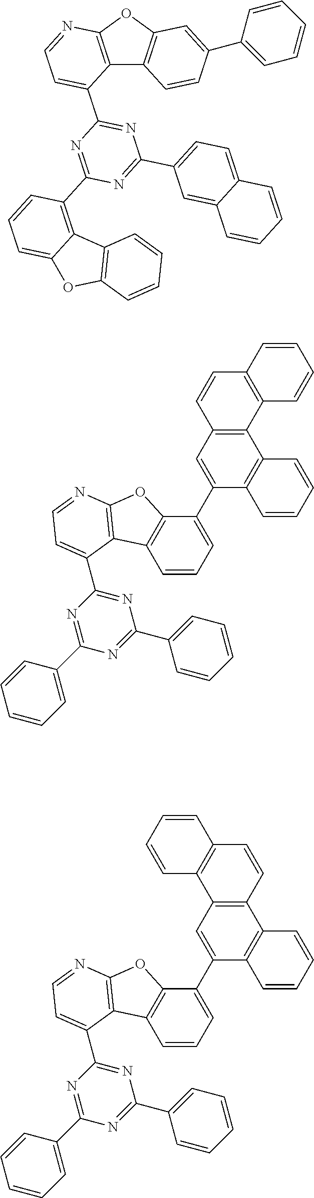 [SMILES: c1ccc(-c2ccc3c(c2)oc2nccc(-c4nc(-c5ccc6ccccc6c5)nc(-c5cccc6oc7ccccc7c56)n4)c23)cc1 ; c1ccc(-c2nc(-c3ccccc3)nc(-c3ccnc4oc5c(-c6cc7ccc8ccccc8c7c7ccccc67)cccc5c34)n2)cc1 ; c1ccc(-c2nc(-c3ccccc3)nc(-c3ccnc4oc5c(-c6cc7c8ccccc8ccc7c7ccccc67)cccc5c34)n2)cc1]

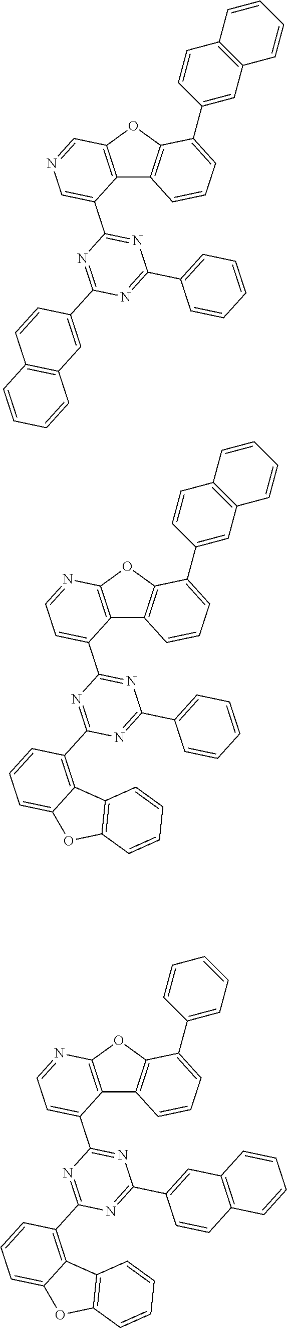 [SMILES: c1ccc(-c2nc(-c3ccc4ccccc4c3)nc(-c3cncc4oc5c(-c6ccc7ccccc7c6)cccc5c34)n2)cc1 ; c1ccc(-c2nc(-c3cccc4oc5ccccc5c34)nc(-c3ccnc4oc5c(-c6ccc7ccccc7c6)cccc5c34)n2)cc1 ; c1ccc(-c2cccc3c2oc2nccc(-c4nc(-c5ccc6ccccc6c5)nc(-c5cccc6oc7ccccc7c56)n4)c23)cc1]

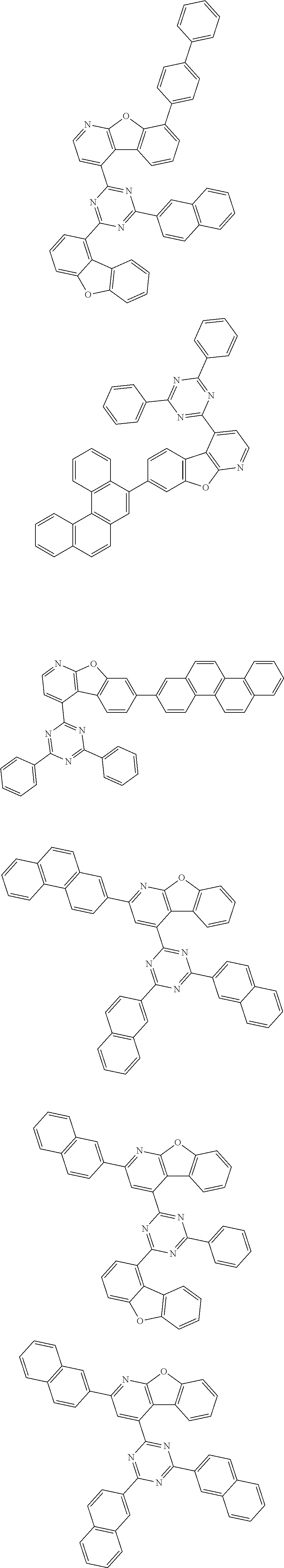

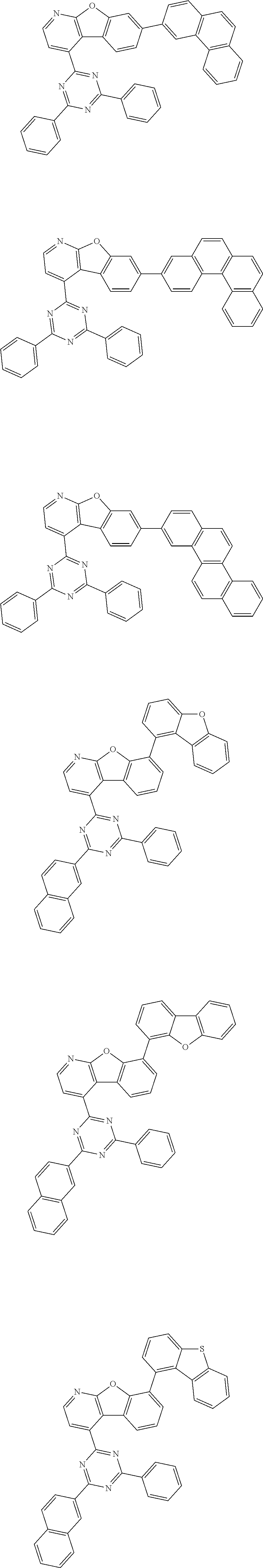 [SMILES: c1ccc(-c2nc(-c3ccccc3)nc(-c3ccnc4oc5cc(-c6ccc7ccc8ccccc8c7c6)ccc5c34)n2)cc1 ; c1ccc(-c2nc(-c3ccccc3)nc(-c3ccnc4oc5cc(-c6ccc7c(ccc8ccc9ccccc9c87)c6)ccc5c34)n2)cc1 ; c1ccc(-c2nc(-c3ccccc3)nc(-c3ccnc4oc5cc(-c6ccc7ccc8c9ccccc9ccc8c7c6)ccc5c34)n2)cc1 ; c1ccc(-c2nc(-c3ccc4ccccc4c3)nc(-c3ccnc4oc5c(-c6cccc7oc8ccccc8c67)cccc5c34)n2)cc1 ; c1ccc(-c2nc(-c3ccc4ccccc4c3)nc(-c3ccnc4oc5c(-c6cccc7c6oc6ccccc67)cccc5c34)n2)cc1 ; c1ccc(-c2nc(-c3ccc4ccccc4c3)nc(-c3ccnc4oc5c(-c6cccc7sc8ccccc8c67)cccc5c34)n2)cc1]

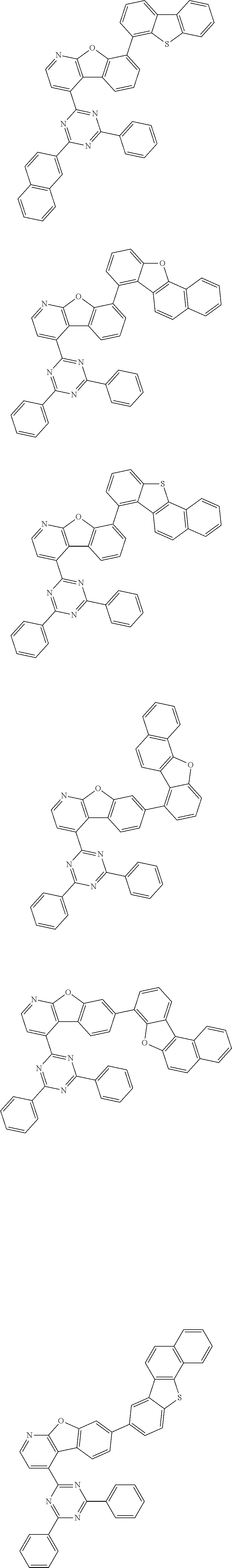 [SMILES: c1ccc(-c2nc(-c3ccc4ccccc4c3)nc(-c3ccnc4oc5c(-c6cccc7c6sc6ccccc67)cccc5c34)n2)cc1 ; c1ccc(-c2nc(-c3ccccc3)nc(-c3ccnc4oc5c(-c6cccc7oc8c9ccccc9ccc8c67)cccc5c34)n2)cc1 ; c1ccc(-c2nc(-c3ccccc3)nc(-c3ccnc4oc5c(-c6cccc7sc8c9ccccc9ccc8c67)cccc5c34)n2)cc1 ; c1ccc(-c2nc(-c3ccccc3)nc(-c3ccnc4oc5cc(-c6cccc7oc8c9ccccc9ccc8c67)ccc5c34)n2)cc1 ; c1ccc(-c2nc(-c3ccccc3)nc(-c3ccnc4oc5cc(-c6cccc7c6oc6ccc8ccccc8c67)ccc5c34)n2)cc1 ; c1ccc(-c2nc(-c3ccccc3)nc(-c3ccnc4oc5cc(-c6ccc7sc8c9ccccc9ccc8c7c6)ccc5c34)n2)cc1]

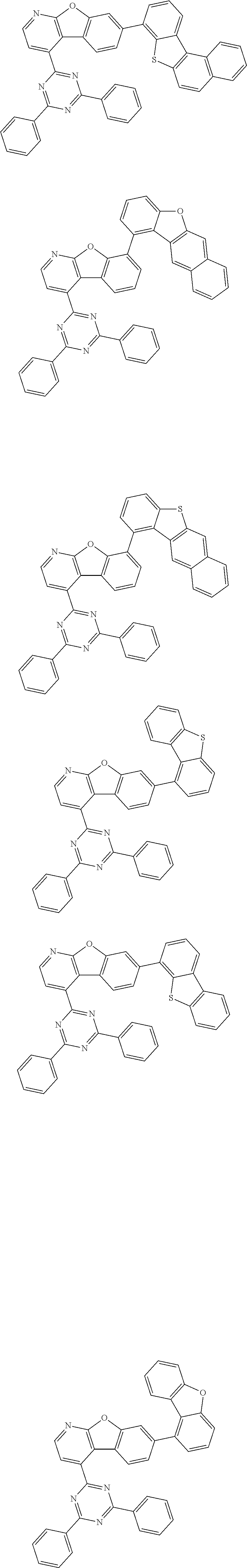 [SMILES: c1ccc(-c2nc(-c3ccccc3)nc(-c3ccnc4oc5cc(-c6cccc7c6sc6ccc8ccccc8c67)ccc5c34)n2)cc1 ; c1ccc(-c2nc(-c3ccccc3)nc(-c3ccnc4oc5c(-c6cccc7oc8cc9ccccc9cc8c67)cccc5c34)n2)cc1 ; c1ccc(-c2nc(-c3ccccc3)nc(-c3ccnc4oc5c(-c6cccc7sc8cc9ccccc9cc8c67)cccc5c34)n2)cc1 ; c1ccc(-c2nc(-c3ccccc3)nc(-c3ccnc4oc5cc(-c6cccc7sc8ccccc8c67)ccc5c34)n2)cc1 ; c1ccc(-c2nc(-c3ccccc3)nc(-c3ccnc4oc5cc(-c6cccc7c6sc6ccccc67)ccc5c34)n2)cc1 ; c1ccc(-c2nc(-c3ccccc3)nc(-c3ccnc4oc5cc(-c6cccc7oc8ccccc8c67)ccc5c34)n2)cc1]

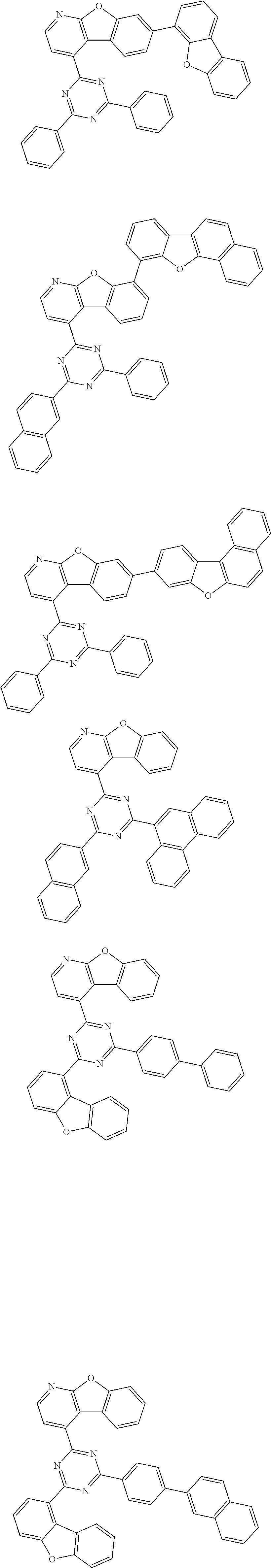 [SMILES: c1ccc(-c2nc(-c3ccccc3)nc(-c3ccnc4oc5cc(-c6cccc7c6oc6ccccc67)ccc5c34)n2)cc1 ; c1ccc(-c2nc(-c3ccc4ccccc4c3)nc(-c3ccnc4oc5c(-c6cccc7c6oc6c8ccccc8ccc76)cccc5c34)n2)cc1 ; c1ccc(-c2nc(-c3ccccc3)nc(-c3ccnc4oc5cc(-c6ccc7c(c6)oc6ccc8ccccc8c67)ccc5c34)n2)cc1 ; c1ccc2cc(-c3nc(-c4cc5ccccc5c5ccccc45)nc(-c4ccnc5oc6ccccc6c45)n3)ccc2c1 ; c1ccc(-c2ccc(-c3nc(-c4cccc5oc6ccccc6c45)nc(-c4ccnc5oc6ccccc6c45)n3)cc2)cc1 ; c1ccc2cc(-c3ccc(-c4nc(-c5cccc6oc7ccccc7c56)nc(-c5ccnc6oc7ccccc7c56)n4)cc3)ccc2c1]

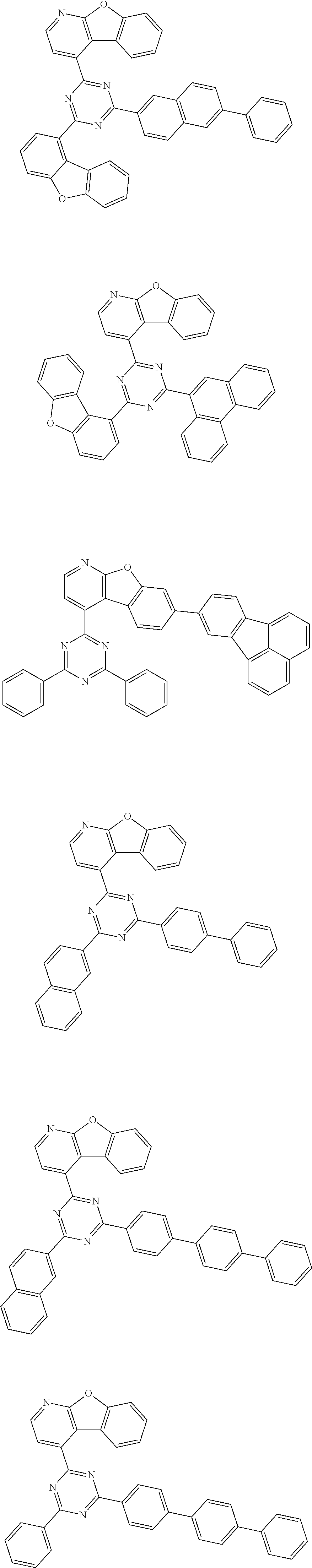 [SMILES: c1ccc(-c2ccc3cc(-c4nc(-c5cccc6oc7ccccc7c56)nc(-c5ccnc6oc7ccccc7c56)n4)ccc3c2)cc1 ; c1ccc2c(c1)cc(-c1nc(-c3cccc4oc5ccccc5c34)nc(-c3ccnc4oc5ccccc5c34)n1)c1ccccc12 ; c1ccc(-c2nc(-c3ccccc3)nc(-c3ccnc4oc5cc(-c6ccc7c(c6)-c6cccc8cccc-7c68)ccc5c34)n2)cc1 ; c1ccc(-c2ccc(-c3nc(-c4ccc5ccccc5c4)nc(-c4ccnc5oc6ccccc6c45)n3)cc2)cc1 ; c1ccc(-c2ccc(-c3ccc(-c4nc(-c5ccc6ccccc6c5)nc(-c5ccnc6oc7ccccc7c56)n4)cc3)cc2)cc1 ; c1ccc(-c2ccc(-c3ccc(-c4nc(-c5ccccc5)nc(-c5ccnc6oc7ccccc7c56)n4)cc3)cc2)cc1]

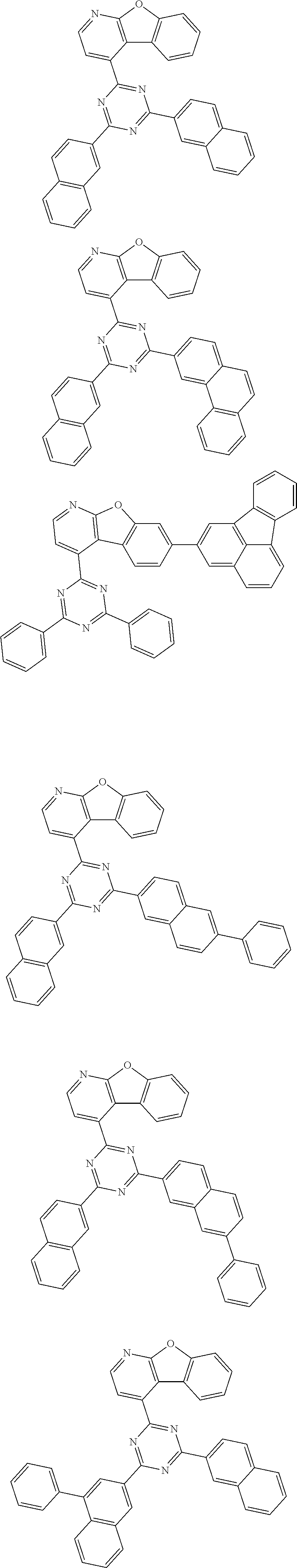

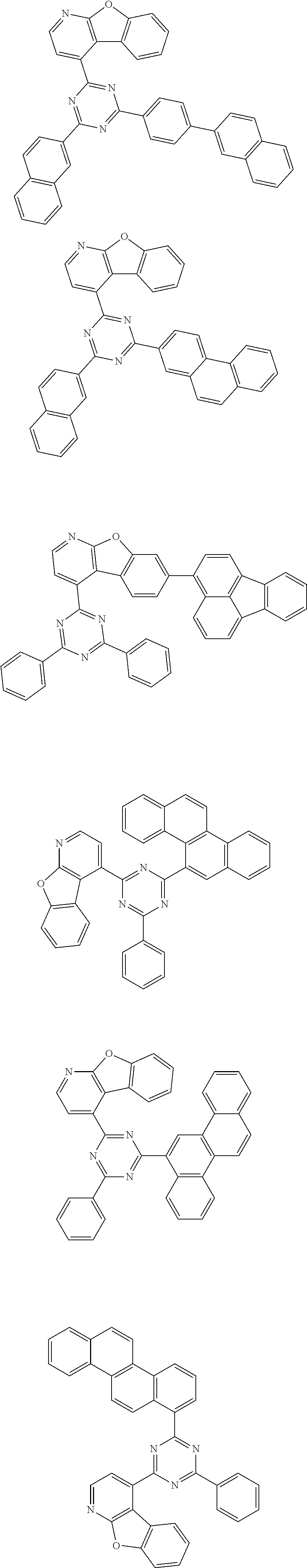 [SMILES: c1ccc2cc(-c3ccc(-c4nc(-c5ccc6ccccc6c5)nc(-c5ccnc6oc7ccccc7c56)n4)cc3)ccc2c1 ; c1ccc2cc(-c3nc(-c4ccc5c(ccc6ccccc65)c4)nc(-c4ccnc5oc6ccccc6c45)n3)ccc2c1 ; c1ccc(-c2nc(-c3ccccc3)nc(-c3ccnc4oc5cc(-c6ccc7c8c(cccc68)-c6ccccc6-7)ccc5c34)n2)cc1 ; c1ccc(-c2nc(-c3ccnc4oc5ccccc5c34)nc(-c3cc4ccccc4c4ccc5ccccc5c34)n2)cc1 ; c1ccc(-c2nc(-c3cc4c5ccccc5ccc4c4ccccc34)nc(-c3ccnc4oc5ccccc5c34)n2)cc1 ; c1ccc(-c2nc(-c3cccc4c3ccc3c5ccccc5ccc43)nc(-c3ccnc4oc5ccccc5c34)n2)cc1]

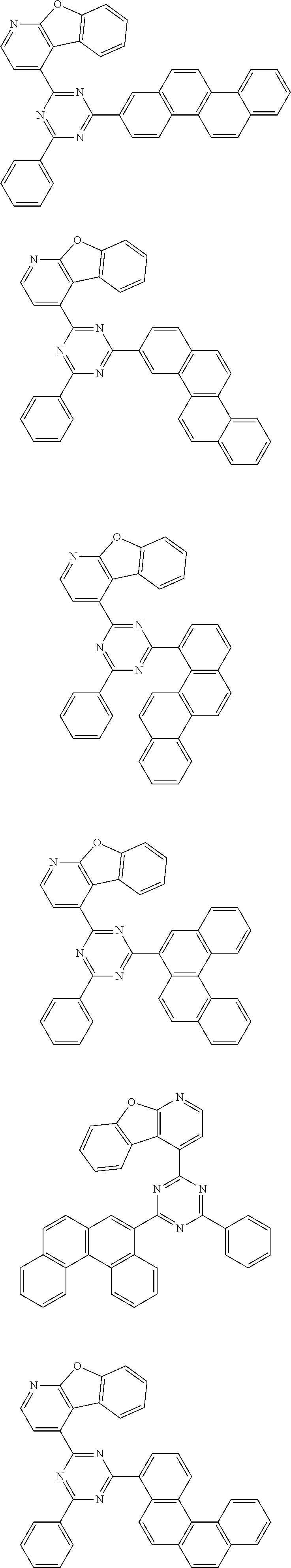 [SMILES: c1ccc(-c2nc(-c3ccc4c(ccc5c6ccccc6ccc45)c3)nc(-c3ccnc4oc5ccccc5c34)n2)cc1 ; c1ccc(-c2nc(-c3ccc4ccc5c6ccccc6ccc5c4c3)nc(-c3ccnc4oc5ccccc5c34)n2)cc1 ; c1ccc(-c2nc(-c3cccc4ccc5c6ccccc6ccc5c34)nc(-c3ccnc4oc5ccccc5c34)n2)cc1 ; c1ccc(-c2nc(-c3cc4ccccc4c4c3ccc3ccccc34)nc(-c3ccnc4oc5ccccc5c34)n2)cc1 ; c1ccc(-c2nc(-c3cc4ccc5ccccc5c4c4ccccc34)nc(-c3ccnc4oc5ccccc5c34)n2)cc1 ; c1ccc(-c2nc(-c3cccc4c3ccc3ccc5ccccc5c34)nc(-c3ccnc4oc5ccccc5c34)n2)cc1]

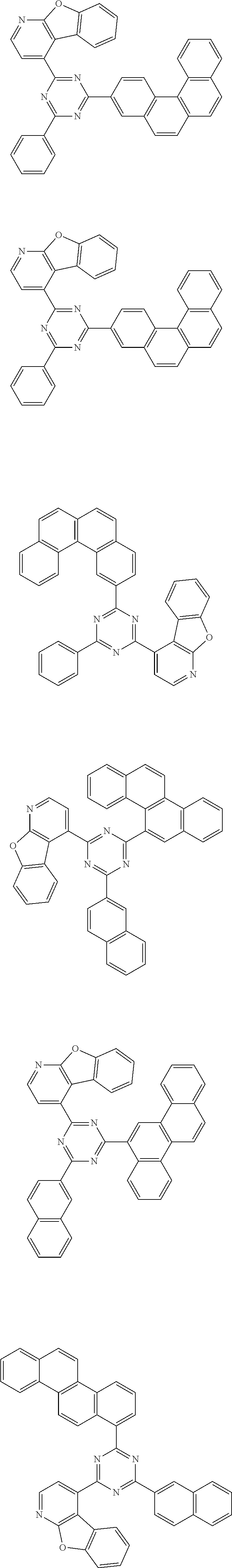 [SMILES: c1ccc(-c2nc(-c3ccc4c(ccc5ccc6ccccc6c54)c3)nc(-c3ccnc4oc5ccccc5c34)n2)cc1 ; c1ccc(-c2nc(-c3ccc4c(ccc5ccc6ccccc6c54)c3)nc(-c3ccnc4oc5ccccc5c34)n2)cc1 ; c1ccc(-c2nc(-c3ccc4ccc5ccc6ccccc6c5c4c3)nc(-c3ccnc4oc5ccccc5c34)n2)cc1 ; c1ccc2cc(-c3nc(-c4ccnc5oc6ccccc6c45)nc(-c4cc5ccccc5c5ccc6ccccc6c45)n3)ccc2c1 ; c1ccc2cc(-c3nc(-c4cc5c6ccccc6ccc5c5ccccc45)nc(-c4ccnc5oc6ccccc6c45)n3)ccc2c1 ; c1ccc2cc(-c3nc(-c4cccc5c4ccc4c6ccccc6ccc54)nc(-c4ccnc5oc6ccccc6c45)n3)ccc2c1]

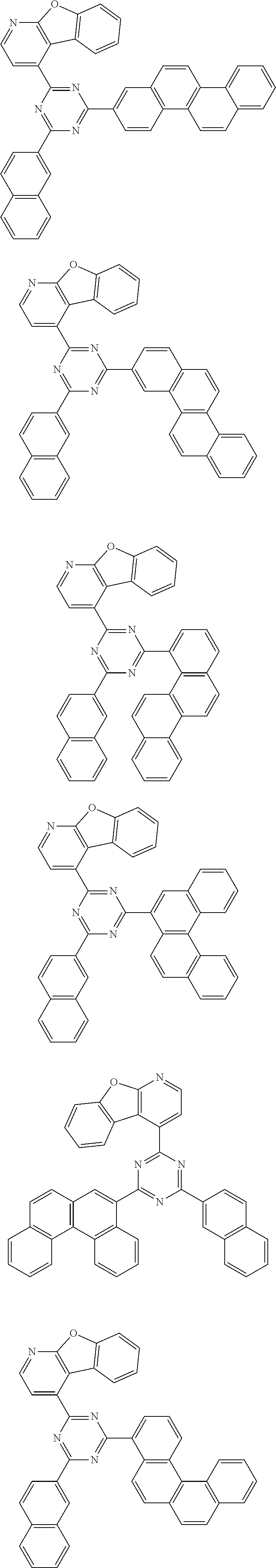

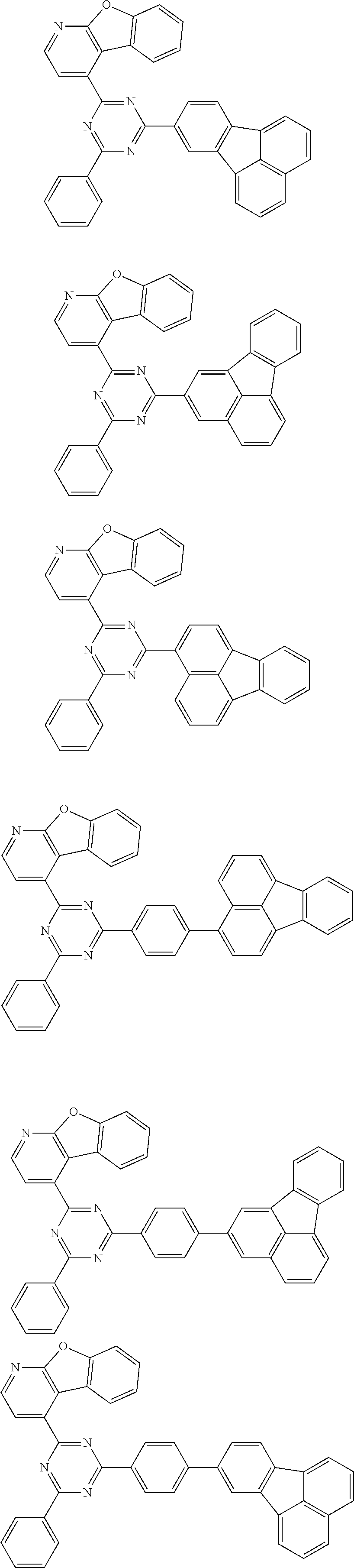

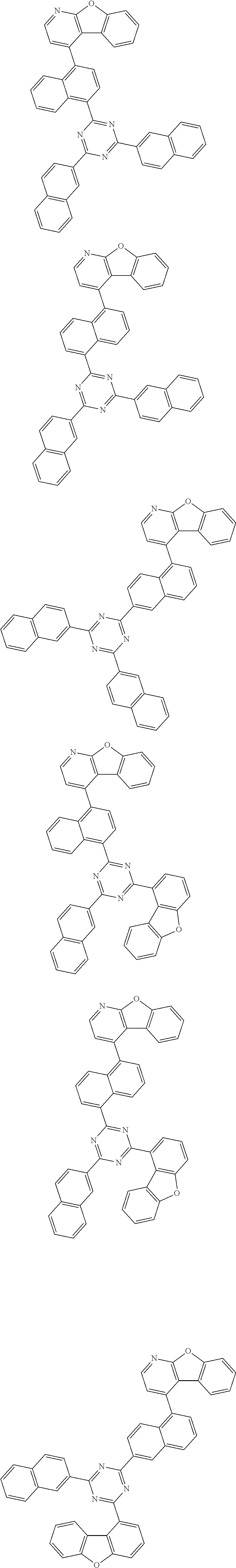

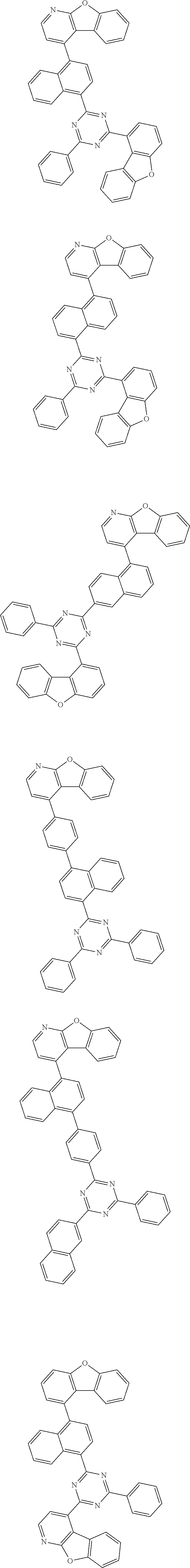

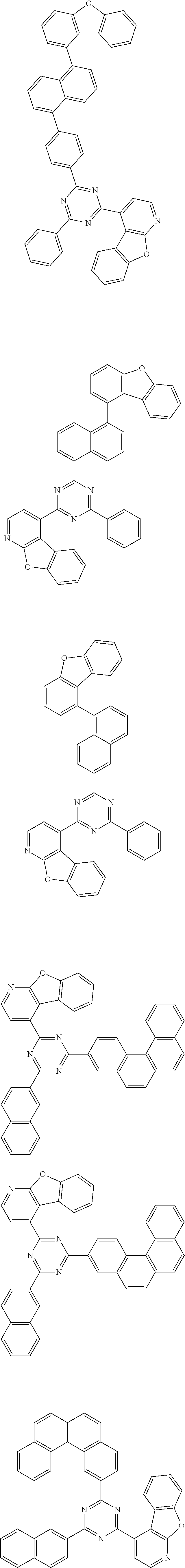

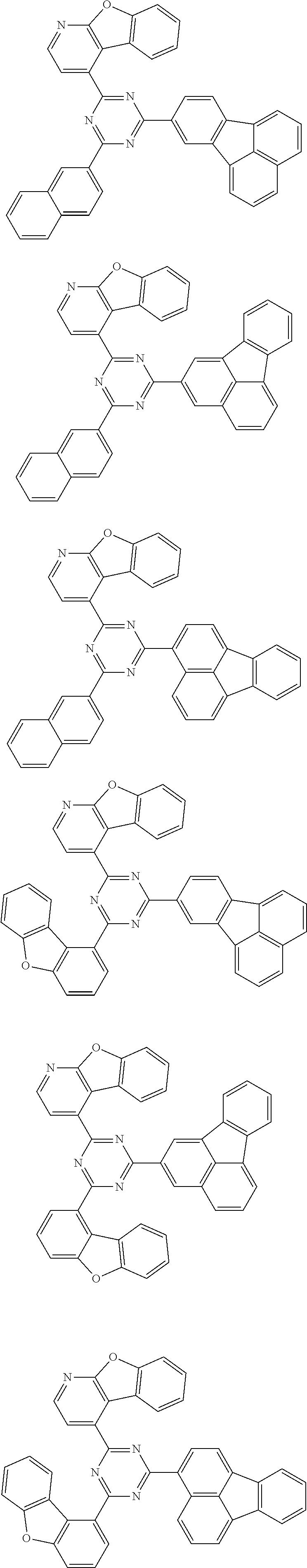 [SMILES: c1ccc2cc(-c3nc(-c4ccc5c(c4)-c4cccc6cccc-5c46)nc(-c4ccnc5oc6ccccc6c45)n3)ccc2c1 ; c1ccc2c(c1)-c1cccc3cc(-c4nc(-c5ccc6ccccc6c5)nc(-c5ccnc6oc7ccccc7c56)n4)cc-2c13 ; c1ccc2c(c1)-c1cccc3c(-c4nc(-c5ccc6ccccc6c5)nc(-c5ccnc6oc7ccccc7c56)n4)ccc-2c13 ; c1cc2c3c(cccc3c1)-c1cc(-c3nc(-c4cccc5oc6ccccc6c45)nc(-c4ccnc5oc6ccccc6c45)n3)ccc1-2 ; c1ccc2c(c1)-c1cccc3cc(-c4nc(-c5cccc6oc7ccccc7c56)nc(-c5ccnc6oc7ccccc7c56)n4)cc-2c13 ; c1ccc2c(c1)-c1cccc3c(-c4nc(-c5cccc6oc7ccccc7c56)nc(-c5ccnc6oc7ccccc7c56)n4)ccc-2c13]

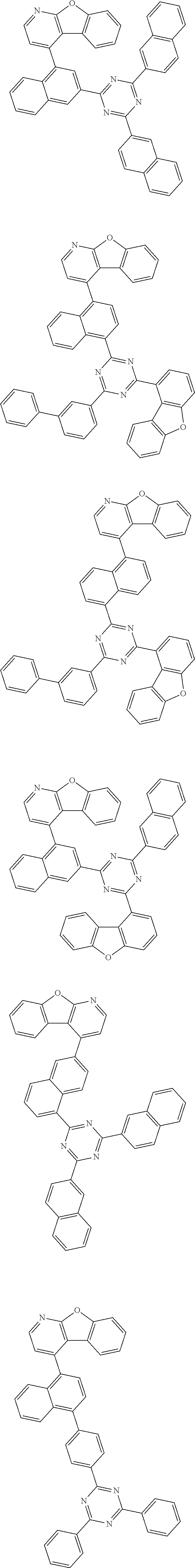

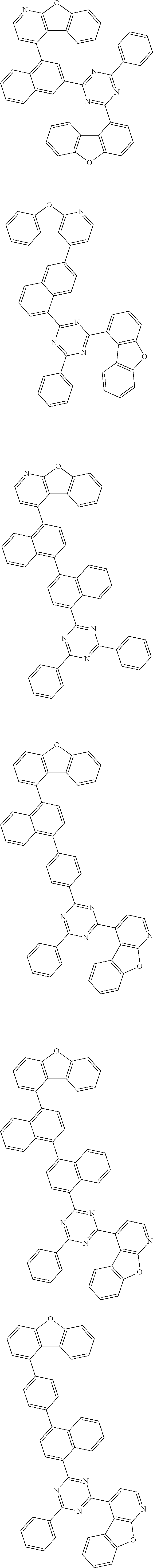

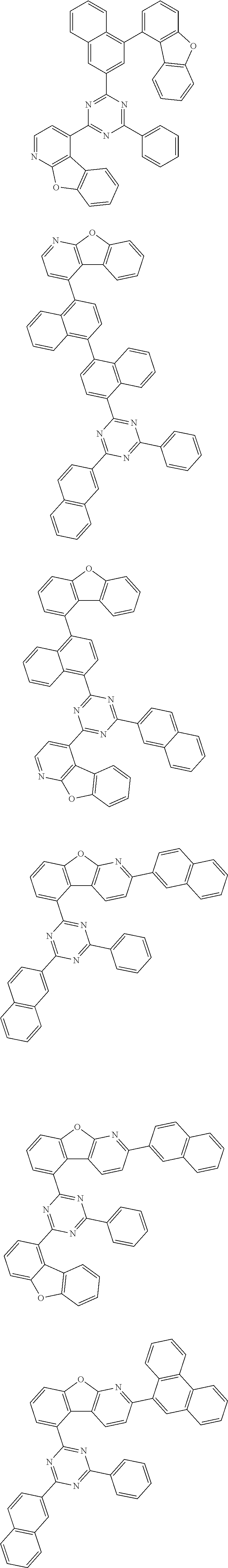

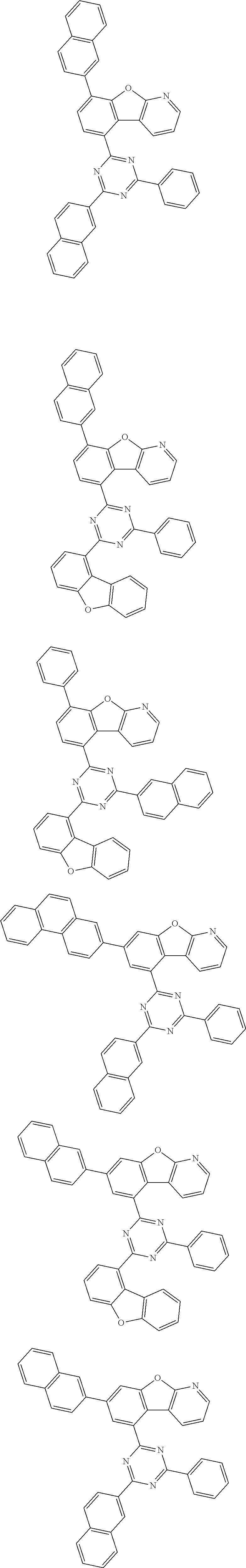

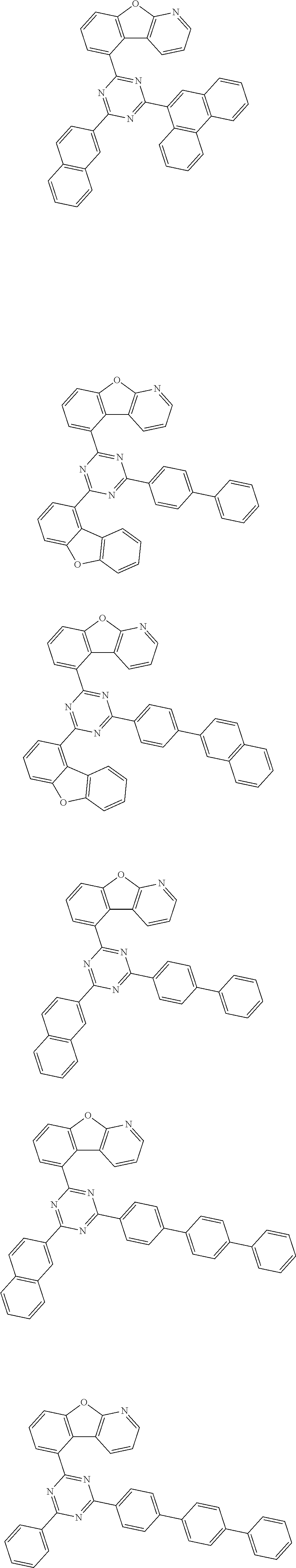 [SMILES: c1ccc2cc(-c3nc(-c4cc5ccccc5c5ccccc45)nc(-c4cccc5oc6ncccc6c45)n3)ccc2c1 ; c1ccc(-c2ccc(-c3nc(-c4cccc5oc6ccccc6c45)nc(-c4cccc5oc6ncccc6c45)n3)cc2)cc1 ; c1ccc2cc(-c3ccc(-c4nc(-c5cccc6oc7ccccc7c56)nc(-c5cccc6oc7ncccc7c56)n4)cc3)ccc2c1 ; c1ccc(-c2ccc(-c3nc(-c4ccc5ccccc5c4)nc(-c4cccc5oc6ncccc6c45)n3)cc2)cc1 ; c1ccc(-c2ccc(-c3ccc(-c4nc(-c5ccc6ccccc6c5)nc(-c5cccc6oc7ncccc7c56)n4)cc3)cc2)cc1 ; c1ccc(-c2ccc(-c3ccc(-c4nc(-c5ccccc5)nc(-c5cccc6oc7ncccc7c56)n4)cc3)cc2)cc1]

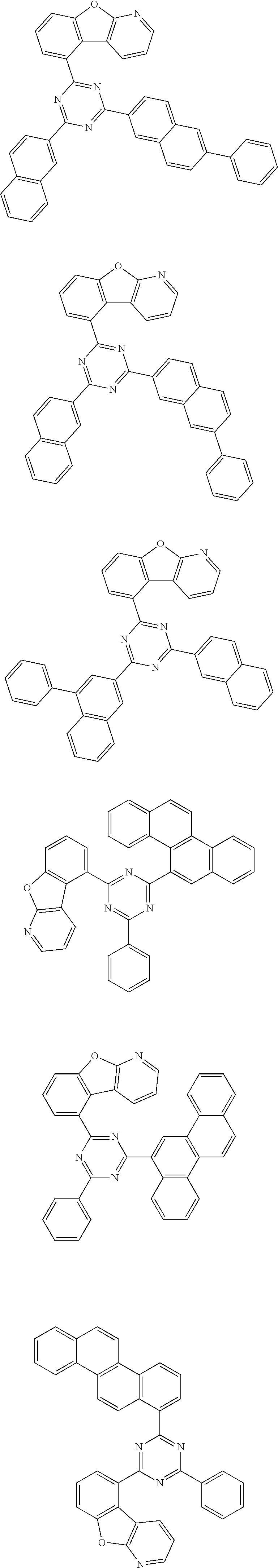 [SMILES: c1ccc(-c2ccc3cc(-c4nc(-c5ccc6ccccc6c5)nc(-c5cccc6oc7ncccc7c56)n4)ccc3c2)cc1 ; c1ccc(-c2ccc3ccc(-c4nc(-c5ccc6ccccc6c5)nc(-c5cccc6oc7ncccc7c56)n4)cc3c2)cc1 ; c1ccc(-c2cc(-c3nc(-c4ccc5ccccc5c4)nc(-c4cccc5oc6ncccc6c45)n3)cc3ccccc23)cc1 ; c1ccc(-c2nc(-c3cccc4oc5ncccc5c34)nc(-c3cc4ccccc4c4ccc5ccccc5c34)n2)cc1 ; c1ccc(-c2nc(-c3cc4c5ccccc5ccc4c4ccccc34)nc(-c3cccc4oc5ncccc5c34)n2)cc1 ; c1ccc(-c2nc(-c3cccc4c3ccc3c5ccccc5ccc43)nc(-c3cccc4oc5ncccc5c34)n2)cc1]

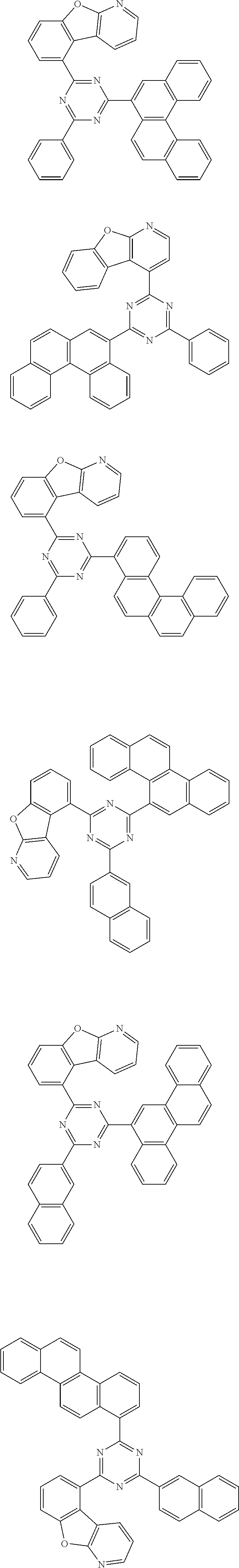 [SMILES: c1ccc(-c2nc(-c3cc4ccccc4c4c3ccc3ccccc34)nc(-c3cccc4oc5ncccc5c34)n2)cc1 ; c1ccc(-c2nc(-c3cc4ccc5ccccc5c4c4ccccc34)nc(-c3ccnc4oc5ccccc5c34)n2)cc1 ; c1ccc(-c2nc(-c3cccc4c3ccc3ccc5ccccc5c34)nc(-c3cccc4oc5ncccc5c34)n2)cc1 ; c1ccc2cc(-c3nc(-c4cccc5oc6ncccc6c45)nc(-c4cc5ccccc5c5ccc6ccccc6c45)n3)ccc2c1 ; c1ccc2cc(-c3nc(-c4cc5c6ccccc6ccc5c5ccccc45)nc(-c4cccc5oc6ncccc6c45)n3)ccc2c1 ; c1ccc2cc(-c3nc(-c4cccc5c4ccc4c6ccccc6ccc54)nc(-c4cccc5oc6ncccc6c45)n3)ccc2c1]

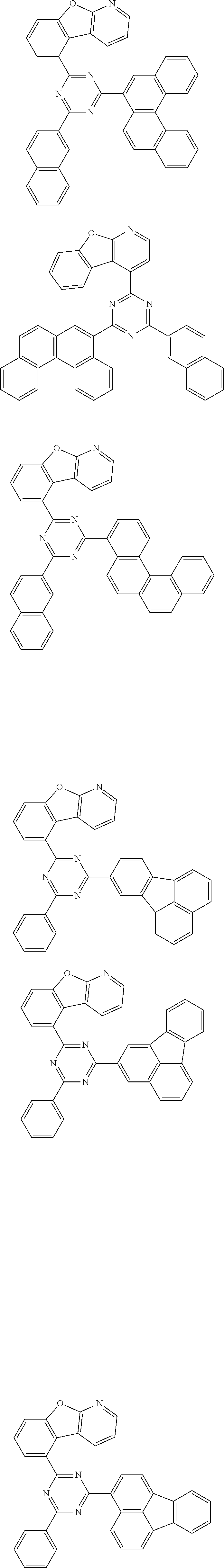 [SMILES: c1ccc2cc(-c3nc(-c4cc5ccccc5c5c4ccc4ccccc45)nc(-c4cccc5oc6ncccc6c45)n3)ccc2c1 ; c1ccc2cc(-c3nc(-c4cc5ccc6ccccc6c5c5ccccc45)nc(-c4ccnc5oc6ccccc6c45)n3)ccc2c1 ; c1ccc2cc(-c3nc(-c4cccc5c4ccc4ccc6ccccc6c45)nc(-c4cccc5oc6ncccc6c45)n3)ccc2c1 ; c1ccc(-c2nc(-c3ccc4c(c3)-c3cccc5cccc-4c35)nc(-c3cccc4oc5ncccc5c34)n2)cc1 ; c1ccc(-c2nc(-c3cc4c5c(cccc5c3)-c3ccccc3-4)nc(-c3cccc4oc5ncccc5c34)n2)cc1 ; c1ccc(-c2nc(-c3ccc4c5c(cccc35)-c3ccccc3-4)nc(-c3cccc4oc5ncccc5c34)n2)cc1]

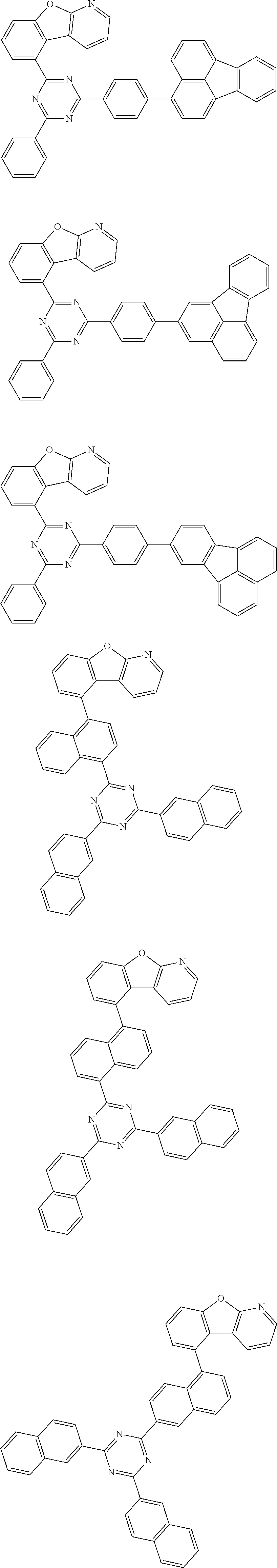

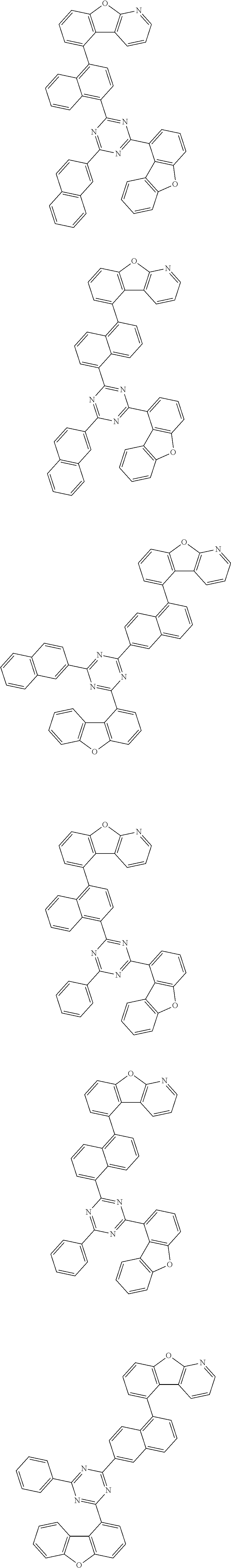

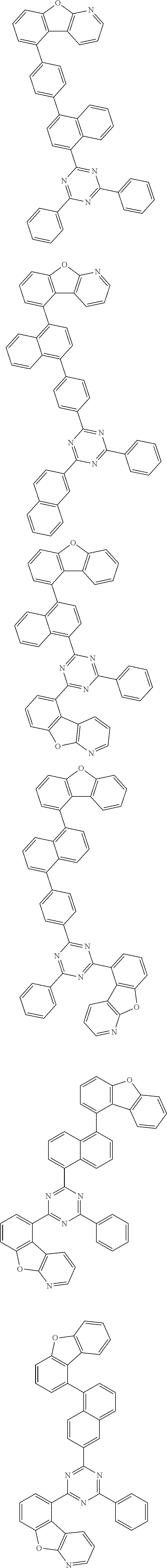

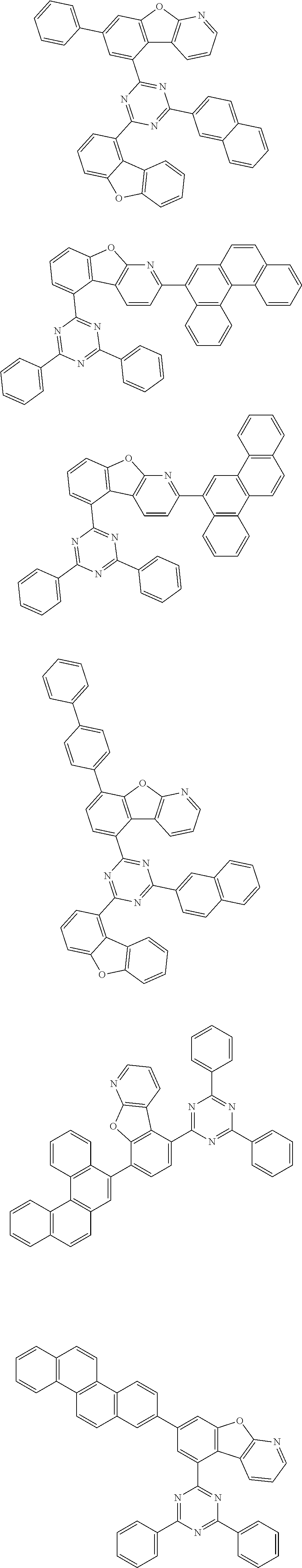

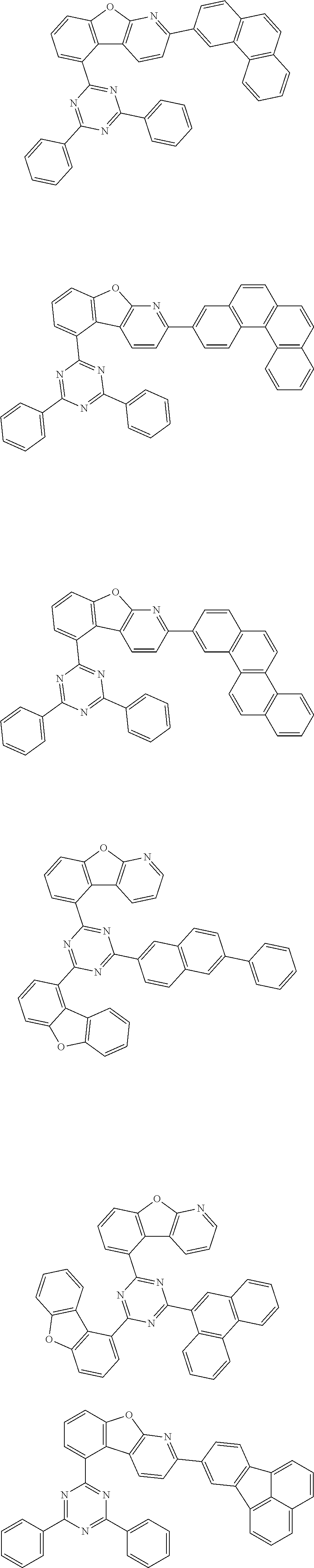 [SMILES: c1ccc(-c2nc(-c3ccccc3)nc(-c3cccc4oc5nc(-c6ccc7ccc8ccccc8c7c6)ccc5c34)n2)cc1 ; c1ccc(-c2nc(-c3ccccc3)nc(-c3cccc4oc5nc(-c6ccc7c(ccc8ccc9ccccc9c87)c6)ccc5c34)n2)cc1 ; c1ccc(-c2nc(-c3ccccc3)nc(-c3cccc4oc5nc(-c6ccc7ccc8c9ccccc9ccc8c7c6)ccc5c34)n2)cc1 ; c1ccc(-c2ccc3cc(-c4nc(-c5cccc6oc7ccccc7c56)nc(-c5cccc6oc7ncccc7c56)n4)ccc3c2)cc1 ; c1ccc2c(c1)cc(-c1nc(-c3cccc4oc5ccccc5c34)nc(-c3cccc4oc5ncccc5c34)n1)c1ccccc12 ; c1ccc(-c2nc(-c3ccccc3)nc(-c3cccc4oc5nc(-c6ccc7c(c6)-c6cccc8cccc-7c68)ccc5c34)n2)cc1]

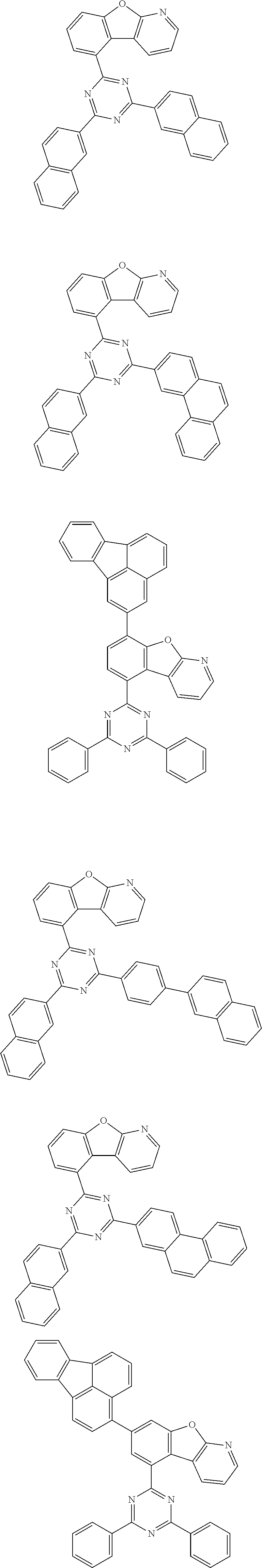

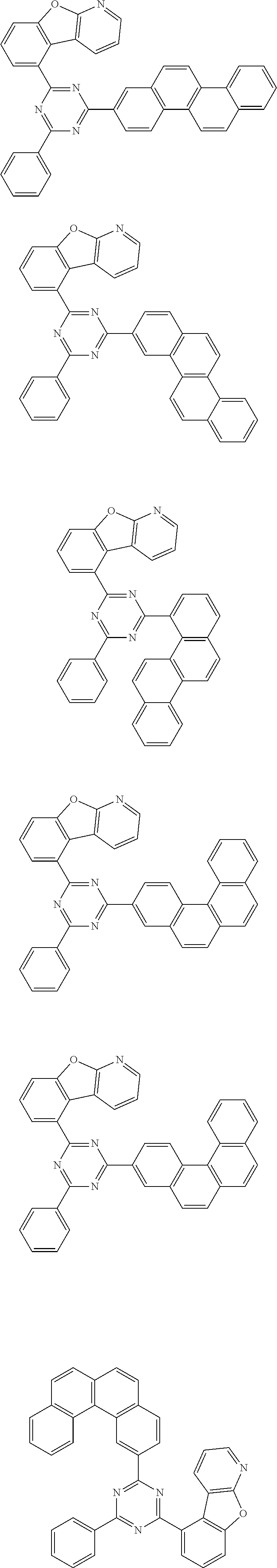 [SMILES: c1ccc(-c2nc(-c3ccc4c(ccc5c6ccccc6ccc45)c3)nc(-c3cccc4oc5ncccc5c34)n2)cc1 ; c1ccc(-c2nc(-c3ccc4ccc5c6ccccc6ccc5c4c3)nc(-c3cccc4oc5ncccc5c34)n2)cc1 ; c1ccc(-c2nc(-c3cccc4ccc5c6ccccc6ccc5c34)nc(-c3cccc4oc5ncccc5c34)n2)cc1 ; c1ccc(-c2nc(-c3ccc4c(ccc5ccc6ccccc6c54)c3)nc(-c3cccc4oc5ncccc5c34)n2)cc1 ; c1ccc(-c2nc(-c3ccc4c(ccc5ccc6ccccc6c54)c3)nc(-c3cccc4oc5ncccc5c34)n2)cc1 ; c1ccc(-c2nc(-c3ccc4ccc5ccc6ccccc6c5c4c3)nc(-c3cccc4oc5ncccc5c34)n2)cc1]

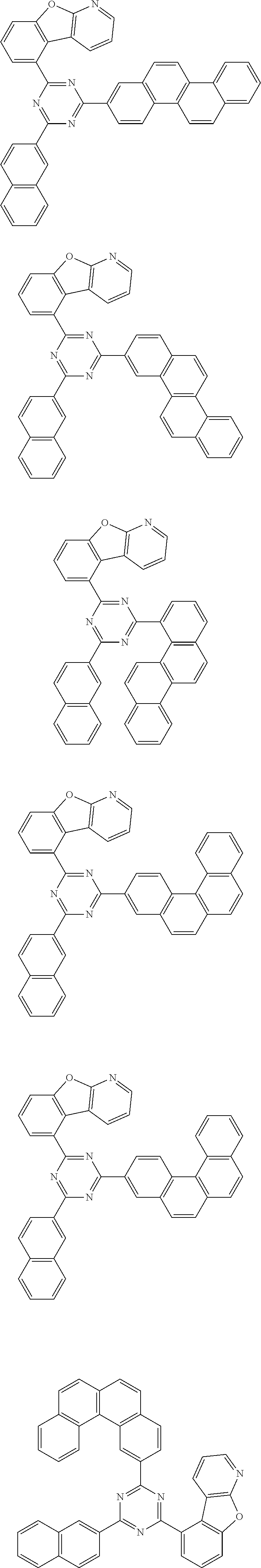 [SMILES: c1ccc2cc(-c3nc(-c4ccc5c(ccc6c7ccccc7ccc56)c4)nc(-c4cccc5oc6ncccc6c45)n3)ccc2c1 ; c1ccc2cc(-c3nc(-c4ccc5ccc6c7ccccc7ccc6c5c4)nc(-c4cccc5oc6ncccc6c45)n3)ccc2c1 ; c1ccc2cc(-c3nc(-c4cccc5ccc6c7ccccc7ccc6c45)nc(-c4cccc5oc6ncccc6c45)n3)ccc2c1 ; c1ccc2cc(-c3nc(-c4ccc5c(ccc6ccc7ccccc7c65)c4)nc(-c4cccc5oc6ncccc6c45)n3)ccc2c1 ; c1ccc2cc(-c3nc(-c4ccc5c(ccc6ccc7ccccc7c65)c4)nc(-c4cccc5oc6ncccc6c45)n3)ccc2c1 ; c1ccc2cc(-c3nc(-c4ccc5ccc6ccc7ccccc7c6c5c4)nc(-c4cccc5oc6ncccc6c45)n3)ccc2c1]

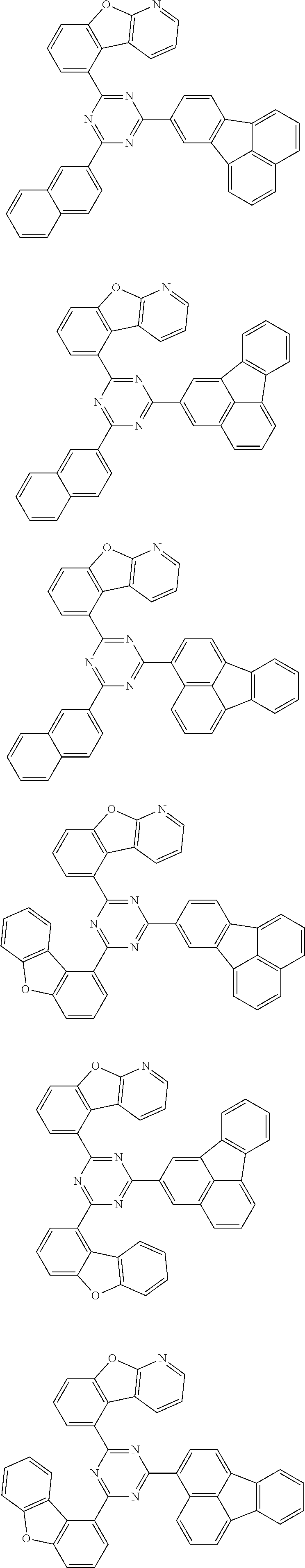 [SMILES: c1ccc2cc(-c3nc(-c4ccc5c(c4)-c4cccc6cccc-5c46)nc(-c4cccc5oc6ncccc6c45)n3)ccc2c1 ; c1ccc2c(c1)-c1cccc3cc(-c4nc(-c5ccc6ccccc6c5)nc(-c5cccc6oc7ncccc7c56)n4)cc-2c13 ; c1ccc2c(c1)-c1cccc3c(-c4nc(-c5ccc6ccccc6c5)nc(-c5cccc6oc7ncccc7c56)n4)ccc-2c13 ; c1cc2c3c(cccc3c1)-c1cc(-c3nc(-c4cccc5oc6ccccc6c45)nc(-c4cccc5oc6ncccc6c45)n3)ccc1-2 ; c1ccc2c(c1)-c1cccc3cc(-c4nc(-c5cccc6oc7ccccc7c56)nc(-c5cccc6oc7ncccc7c56)n4)cc-2c13 ; c1ccc2c(c1)-c1cccc3c(-c4nc(-c5cccc6oc7ccccc7c56)nc(-c5cccc6oc7ncccc7c56)n4)ccc-2c13]

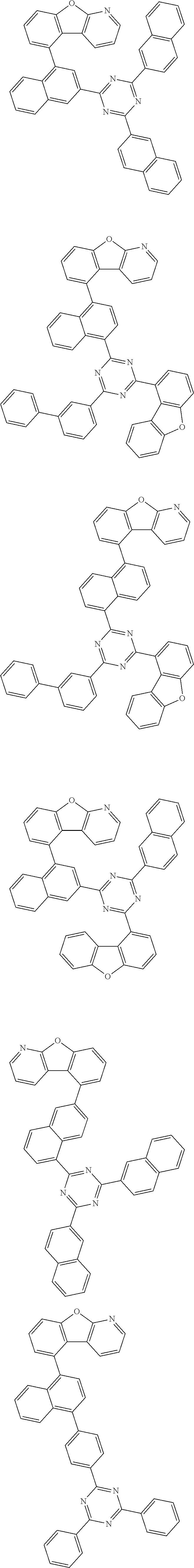

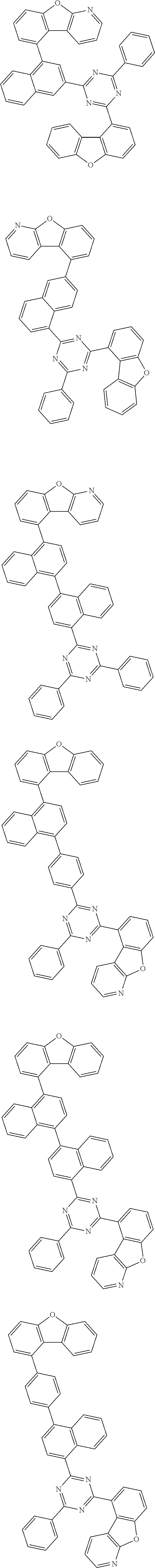

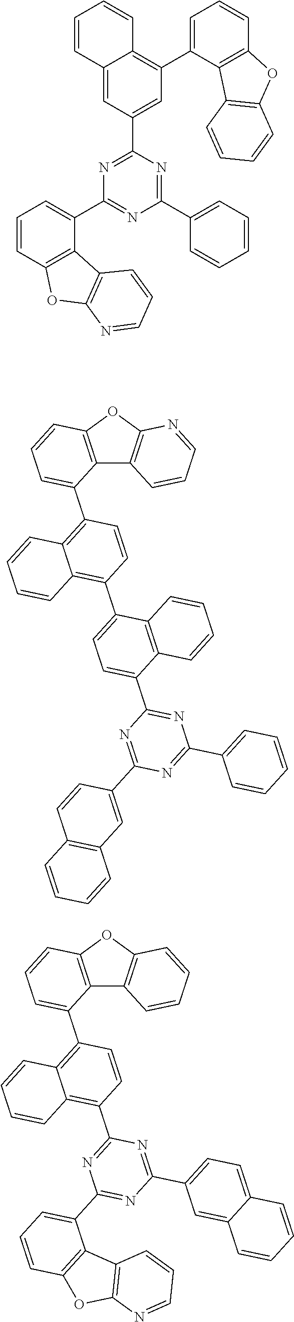

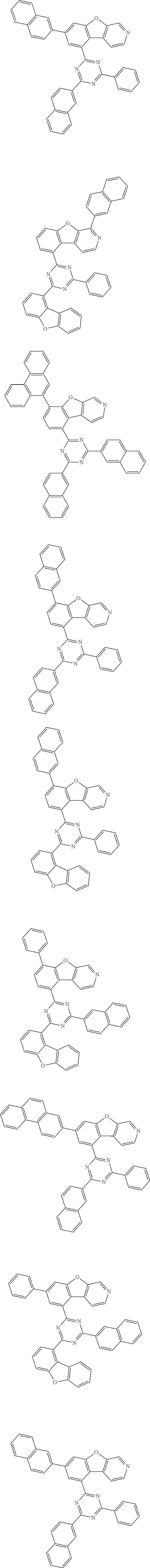

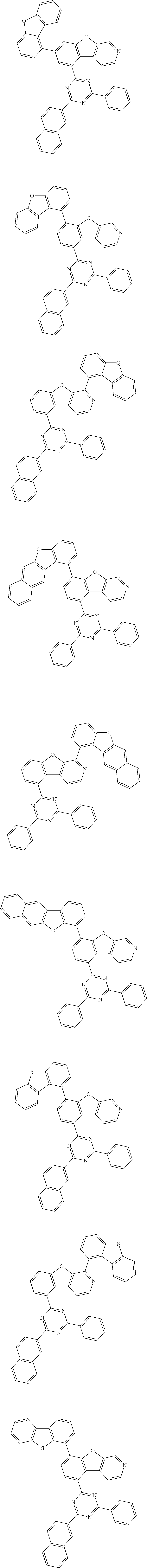

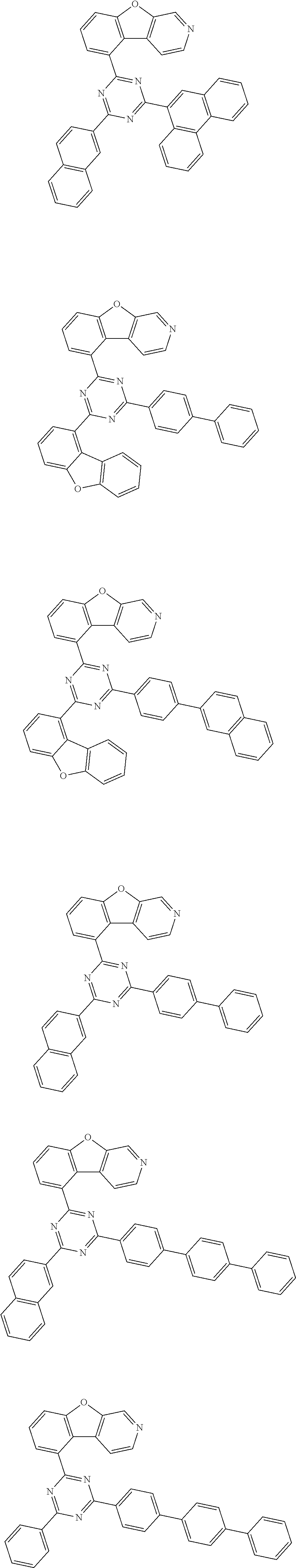 [SMILES: c1ccc2cc(-c3nc(-c4cc5ccccc5c5ccccc45)nc(-c4cccc5oc6cnccc6c45)n3)ccc2c1 ; c1ccc(-c2ccc(-c3nc(-c4cccc5oc6ccccc6c45)nc(-c4cccc5oc6cnccc6c45)n3)cc2)cc1 ; c1ccc2cc(-c3ccc(-c4nc(-c5cccc6oc7ccccc7c56)nc(-c5cccc6oc7cnccc7c56)n4)cc3)ccc2c1 ; c1ccc(-c2ccc(-c3nc(-c4ccc5ccccc5c4)nc(-c4cccc5oc6cnccc6c45)n3)cc2)cc1 ; c1ccc(-c2ccc(-c3ccc(-c4nc(-c5ccc6ccccc6c5)nc(-c5cccc6oc7cnccc7c56)n4)cc3)cc2)cc1 ; c1ccc(-c2ccc(-c3ccc(-c4nc(-c5ccccc5)nc(-c5cccc6oc7cnccc7c56)n4)cc3)cc2)cc1]

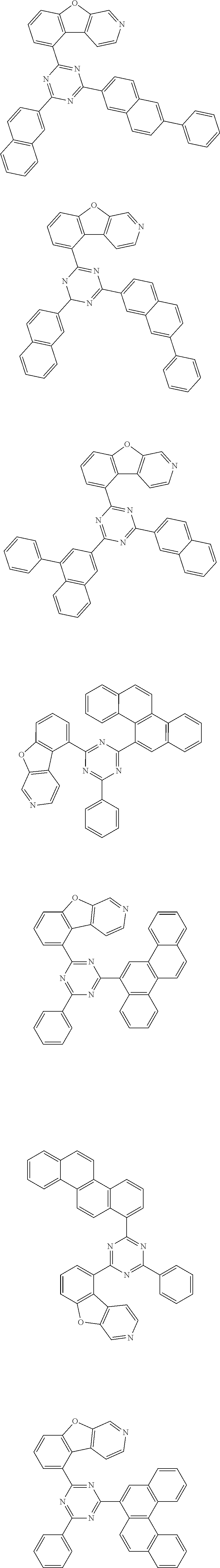

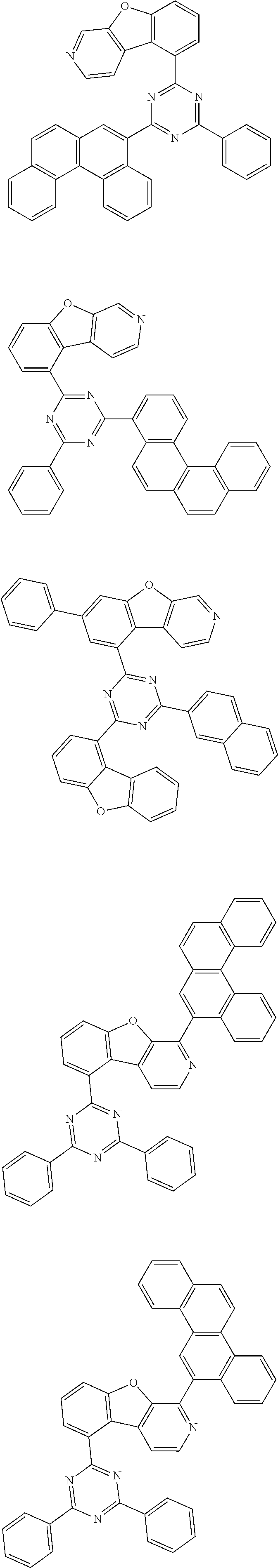 [SMILES: c1ccc(-c2nc(-c3cc4ccc5ccccc5c4c4ccccc34)nc(-c3cccc4oc5cnccc5c34)n2)cc1 ; c1ccc(-c2nc(-c3cccc4c3ccc3ccc5ccccc5c34)nc(-c3cccc4oc5cnccc5c34)n2)cc1 ; c1ccc(-c2cc(-c3nc(-c4ccc5ccccc5c4)nc(-c4cccc5oc6ccccc6c45)n3)c3c(c2)oc2cnccc23)cc1 ; c1ccc(-c2nc(-c3ccccc3)nc(-c3cccc4oc5c(-c6cc7ccc8ccccc8c7c7ccccc67)nccc5c34)n2)cc1 ; c1ccc(-c2nc(-c3ccccc3)nc(-c3cccc4oc5c(-c6cc7c8ccccc8ccc7c7ccccc67)nccc5c34)n2)cc1]

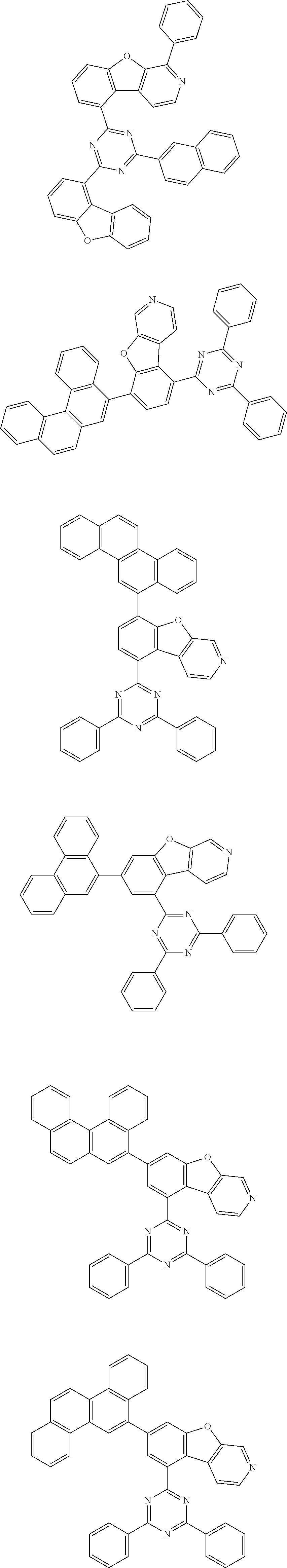 [SMILES: c1ccc(-c2nccc3c2oc2cccc(-c4nc(-c5ccc6ccccc6c5)nc(-c5cccc6oc7ccccc7c56)n4)c23)cc1 ; c1ccc(-c2nc(-c3ccccc3)nc(-c3ccc(-c4cc5ccc6ccccc6c5c5ccccc45)c4oc5cnccc5c34)n2)cc1 ; c1ccc(-c2nc(-c3ccccc3)nc(-c3ccc(-c4cc5c6ccccc6ccc5c5ccccc45)c4oc5cnccc5c34)n2)cc1 ; c1ccc(-c2nc(-c3ccccc3)nc(-c3cc(-c4cc5ccccc5c5ccccc45)cc4oc5cnccc5c34)n2)cc1 ; c1ccc(-c2nc(-c3ccccc3)nc(-c3cc(-c4cc5ccc6ccccc6c5c5ccccc45)cc4oc5cnccc5c34)n2)cc1 ; c1ccc(-c2nc(-c3ccccc3)nc(-c3cc(-c4cc5c6ccccc6ccc5c5ccccc45)cc4oc5cnccc5c34)n2)cc1]

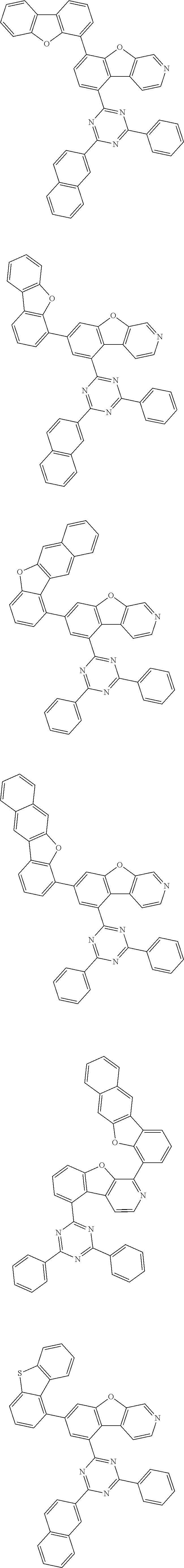 [SMILES: c1ccc(-c2nc(-c3ccc4ccccc4c3)nc(-c3ccc(-c4cccc5c4oc4ccccc45)c4oc5cnccc5c34)n2)cc1 ; c1ccc(-c2nc(-c3ccc4ccccc4c3)nc(-c3cc(-c4cccc5c4oc4ccccc45)cc4oc5cnccc5c34)n2)cc1 ; c1ccc(-c2nc(-c3ccccc3)nc(-c3cc(-c4cccc5oc6cc7ccccc7cc6c45)cc4oc5cnccc5c34)n2)cc1 ; c1ccc(-c2nc(-c3ccccc3)nc(-c3cc(-c4cccc5c4oc4cc6ccccc6cc45)cc4oc5cnccc5c34)n2)cc1 ; c1ccc(-c2nc(-c3ccccc3)nc(-c3cccc4oc5c(-c6cccc7c6oc6cc8ccccc8cc67)nccc5c34)n2)cc1 ; c1ccc(-c2nc(-c3ccc4ccccc4c3)nc(-c3cc(-c4cccc5sc6ccccc6c45)cc4oc5cnccc5c34)n2)cc1]

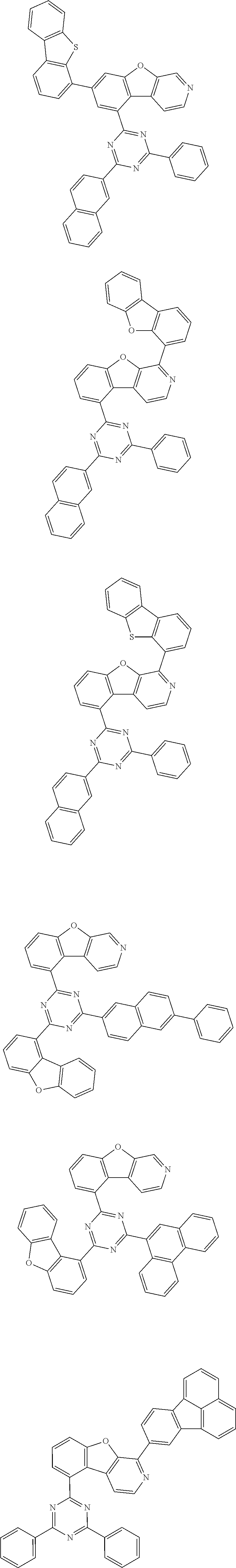 [SMILES: c1ccc(-c2nc(-c3ccc4ccccc4c3)nc(-c3cc(-c4cccc5c4sc4ccccc45)cc4oc5cnccc5c34)n2)cc1 ; c1ccc(-c2nc(-c3ccc4ccccc4c3)nc(-c3cccc4oc5c(-c6cccc7c6oc6ccccc67)nccc5c34)n2)cc1 ; c1ccc(-c2nc(-c3ccc4ccccc4c3)nc(-c3cccc4oc5c(-c6cccc7c6sc6ccccc67)nccc5c34)n2)cc1 ; c1ccc(-c2ccc3cc(-c4nc(-c5cccc6oc7ccccc7c56)nc(-c5cccc6oc7cnccc7c56)n4)ccc3c2)cc1 ; c1ccc2c(c1)cc(-c1nc(-c3cccc4oc5ccccc5c34)nc(-c3cccc4oc5cnccc5c34)n1)c1ccccc12 ; c1ccc(-c2nc(-c3ccccc3)nc(-c3cccc4oc5c(-c6ccc7c(c6)-c6cccc8cccc-7c68)nccc5c34)n2)cc1]

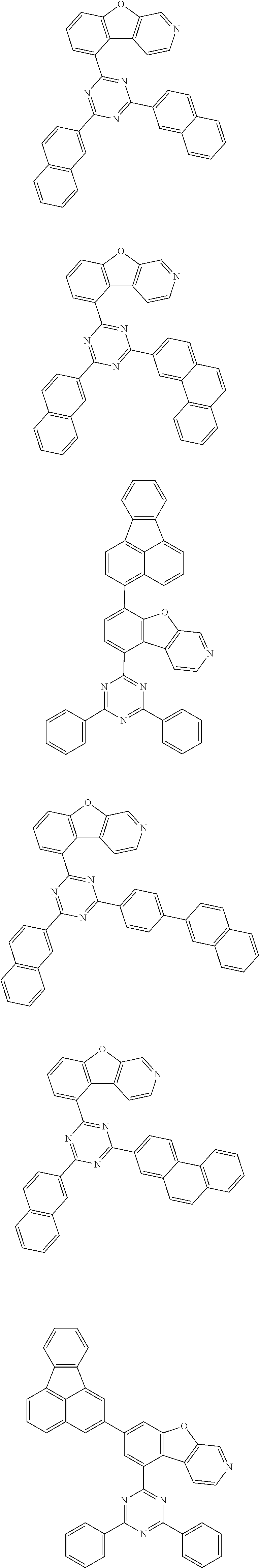 [SMILES: c1ccc2cc(-c3nc(-c4ccc5ccccc5c4)nc(-c4cccc5oc6cnccc6c45)n3)ccc2c1 ; c1ccc2cc(-c3nc(-c4ccc5ccc6ccccc6c5c4)nc(-c4cccc5oc6cnccc6c45)n3)ccc2c1 ; c1ccc(-c2nc(-c3ccccc3)nc(-c3ccc(-c4ccc5c6c(cccc46)-c4ccccc4-5)c4oc5cnccc5c34)n2)cc1 ; c1ccc2cc(-c3ccc(-c4nc(-c5ccc6ccccc6c5)nc(-c5cccc6oc7cnccc7c56)n4)cc3)ccc2c1 ; c1ccc2cc(-c3nc(-c4ccc5c(ccc6ccccc65)c4)nc(-c4cccc5oc6cnccc6c45)n3)ccc2c1 ; c1ccc(-c2nc(-c3ccccc3)nc(-c3cc(-c4cc5c6c(cccc6c4)-c4ccccc4-5)cc4oc5cnccc5c34)n2)cc1]

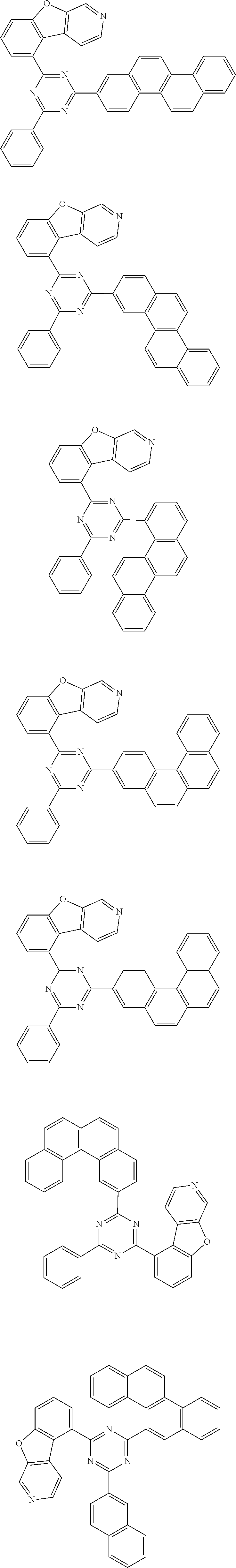

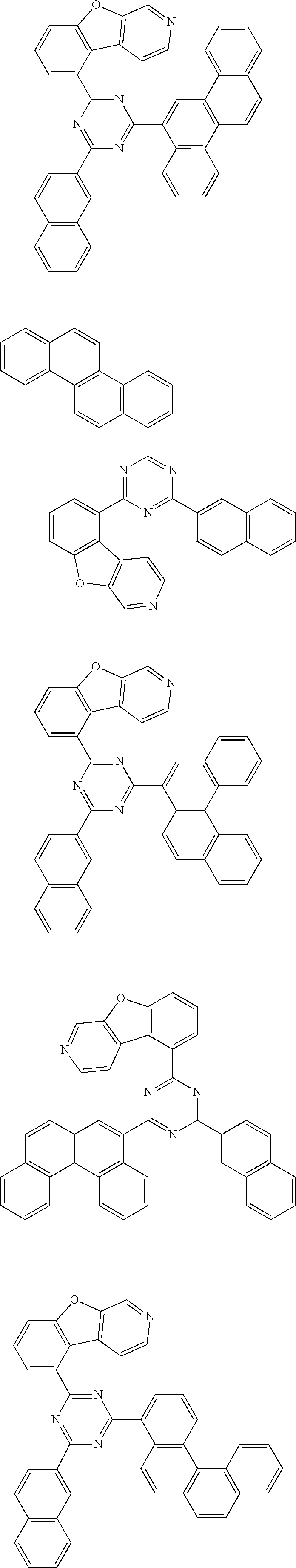 [SMILES: c1ccc2cc(-c3nc(-c4cc5c6ccccc6ccc5c5ccccc45)nc(-c4cccc5oc6cnccc6c45)n3)ccc2c1 ; c1ccc2cc(-c3nc(-c4cccc5c4ccc4c6ccccc6ccc54)nc(-c4cccc5oc6cnccc6c45)n3)ccc2c1 ; c1ccc2cc(-c3nc(-c4cc5ccccc5c5c4ccc4ccccc45)nc(-c4cccc5oc6cnccc6c45)n3)ccc2c1 ; c1ccc2cc(-c3nc(-c4cc5ccc6ccccc6c5c5ccccc45)nc(-c4cccc5oc6cnccc6c45)n3)ccc2c1 ; c1ccc2cc(-c3nc(-c4cccc5c4ccc4ccc6ccccc6c45)nc(-c4cccc5oc6cnccc6c45)n3)ccc2c1]

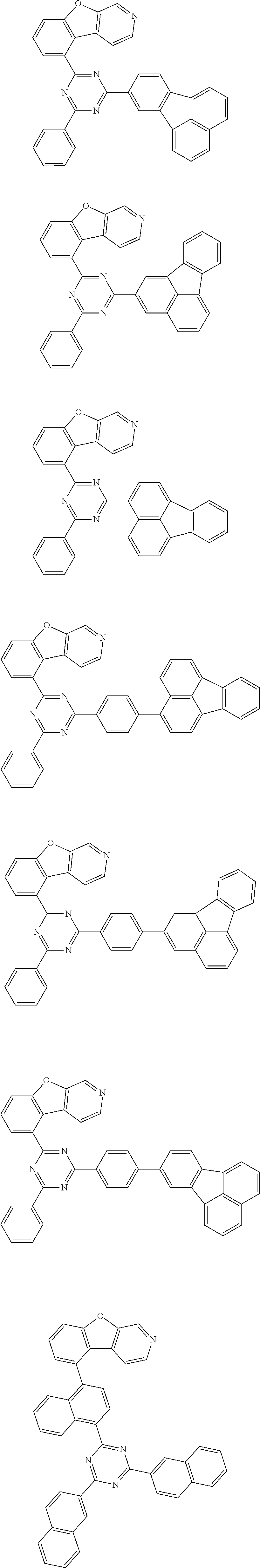

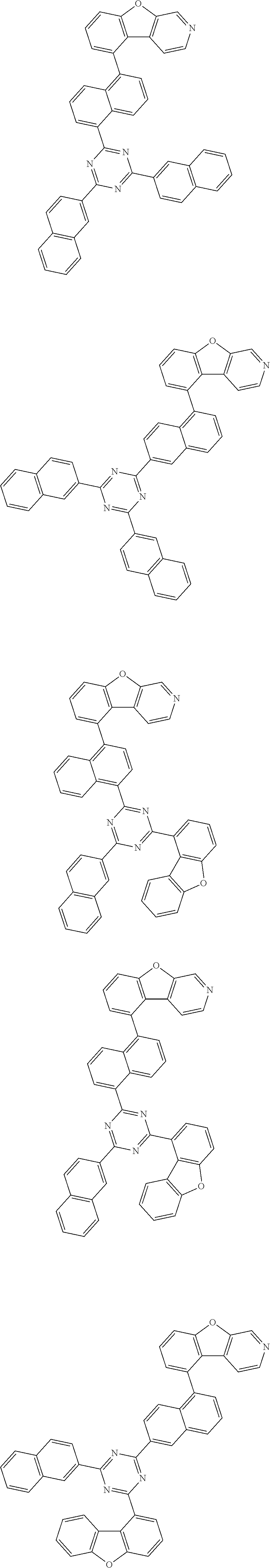 [SMILES: c1ccc2cc(-c3nc(-c4ccc5ccccc5c4)nc(-c4cccc5c(-c6cccc7oc8cnccc8c67)cccc45)n3)ccc2c1 ; c1ccc2cc(-c3nc(-c4ccc5ccccc5c4)nc(-c4ccc5c(-c6cccc7oc8cnccc8c67)cccc5c4)n3)ccc2c1 ; c1ccc2cc(-c3nc(-c4ccc(-c5cccc6oc7cnccc7c56)c5ccccc45)nc(-c4cccc5oc6ccccc6c45)n3)ccc2c1 ; c1ccc2cc(-c3nc(-c4cccc5c(-c6cccc7oc8cnccc8c67)cccc45)nc(-c4cccc5oc6ccccc6c45)n3)ccc2c1 ; c1ccc2cc(-c3nc(-c4ccc5c(-c6cccc7oc8cnccc8c67)cccc5c4)nc(-c4cccc5oc6ccccc6c45)n3)ccc2c1]

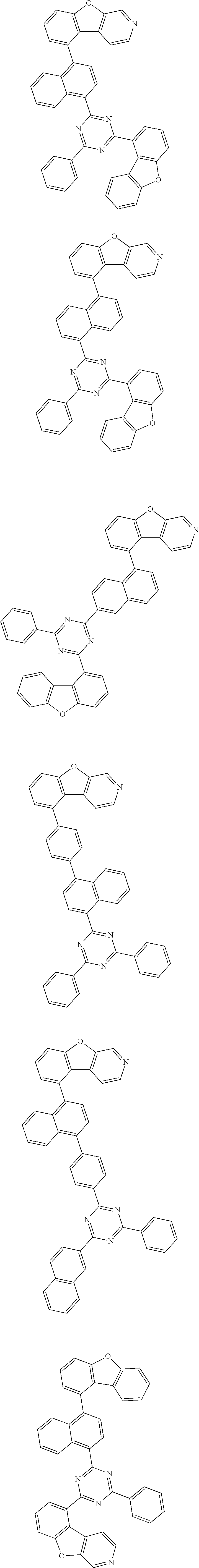

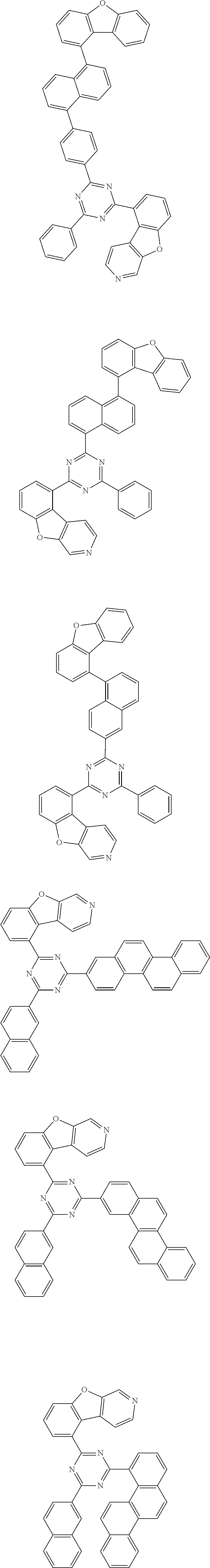 [SMILES: c1ccc(-c2nc(-c3ccc(-c4cccc5c(-c6cccc7oc8ccccc8c67)cccc45)cc3)nc(-c3cccc4oc5cnccc5c34)n2)cc1 ; c1ccc(-c2nc(-c3cccc4c(-c5cccc6oc7ccccc7c56)cccc34)nc(-c3cccc4oc5cnccc5c34)n2)cc1 ; c1ccc(-c2nc(-c3ccc4c(-c5cccc6oc7ccccc7c56)cccc4c3)nc(-c3cccc4oc5cnccc5c34)n2)cc1 ; c1ccc2cc(-c3nc(-c4ccc5c(ccc6c7ccccc7ccc56)c4)nc(-c4cccc5oc6cnccc6c45)n3)ccc2c1 ; c1ccc2cc(-c3nc(-c4ccc5ccc6c7ccccc7ccc6c5c4)nc(-c4cccc5oc6cnccc6c45)n3)ccc2c1 ; c1ccc2cc(-c3nc(-c4cccc5ccc6c7ccccc7ccc6c45)nc(-c4cccc5oc6cnccc6c45)n3)ccc2c1]

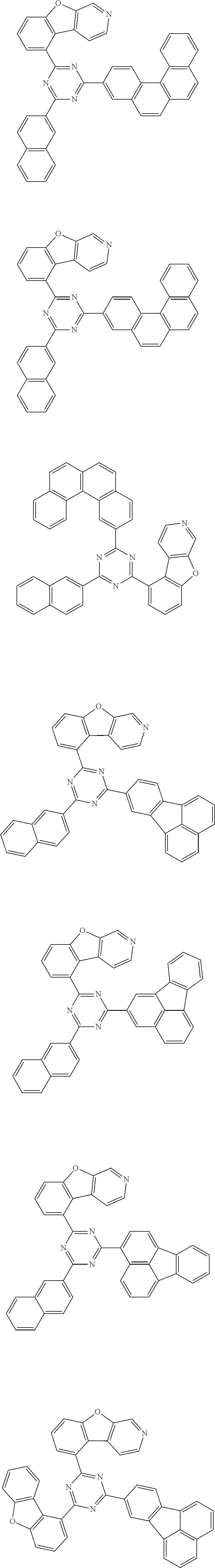

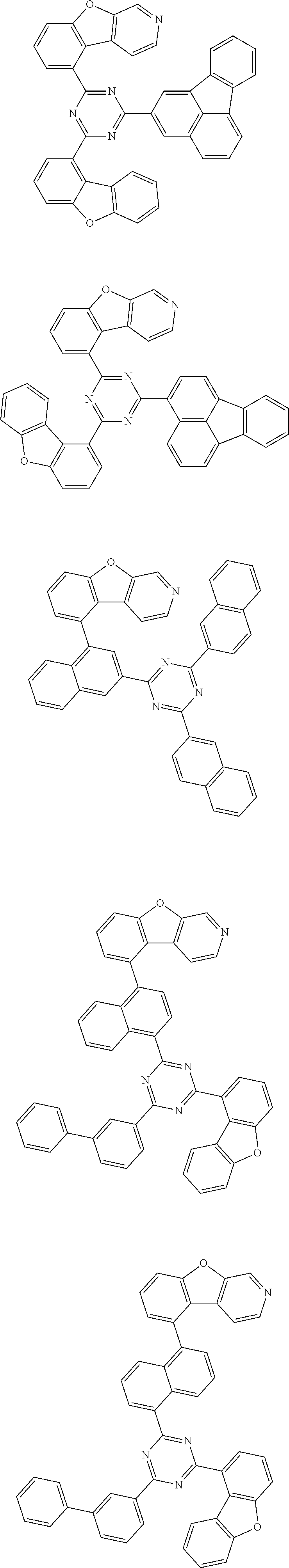 [SMILES: c1ccc2c(c1)-c1cccc3cc(-c4nc(-c5cccc6oc7ccccc7c56)nc(-c5cccc6oc7cnccc7c56)n4)cc-2c13 ; c1ccc2c(c1)-c1cccc3c(-c4nc(-c5cccc6oc7ccccc7c56)nc(-c5cccc6oc7cnccc7c56)n4)ccc-2c13 ; c1ccc2cc(-c3nc(-c4ccc5ccccc5c4)nc(-c4cc(-c5cccc6oc7cnccc7c56)c5ccccc5c4)n3)ccc2c1 ; c1ccc(-c2cccc(-c3nc(-c4ccc(-c5cccc6oc7cnccc7c56)c5ccccc45)nc(-c4cccc5oc6ccccc6c45)n3)c2)cc1 ; c1ccc(-c2cccc(-c3nc(-c4cccc5c(-c6cccc7oc8cnccc8c67)cccc45)nc(-c4cccc5oc6ccccc6c45)n3)c2)cc1]

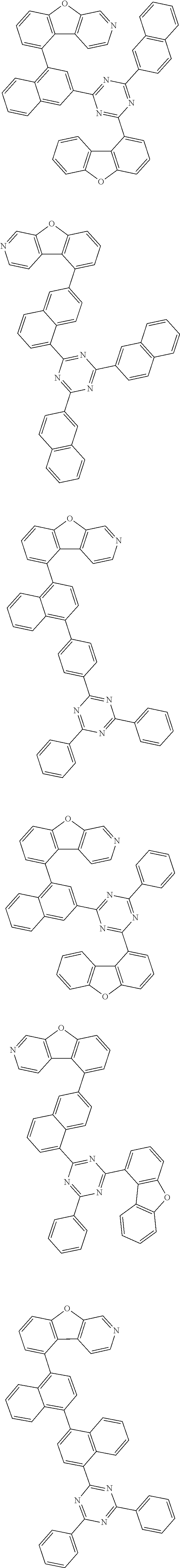

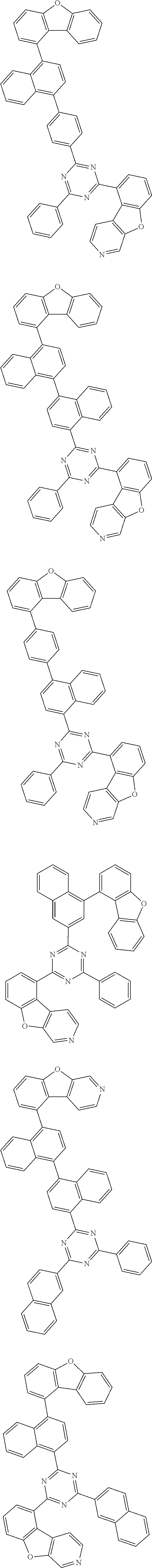

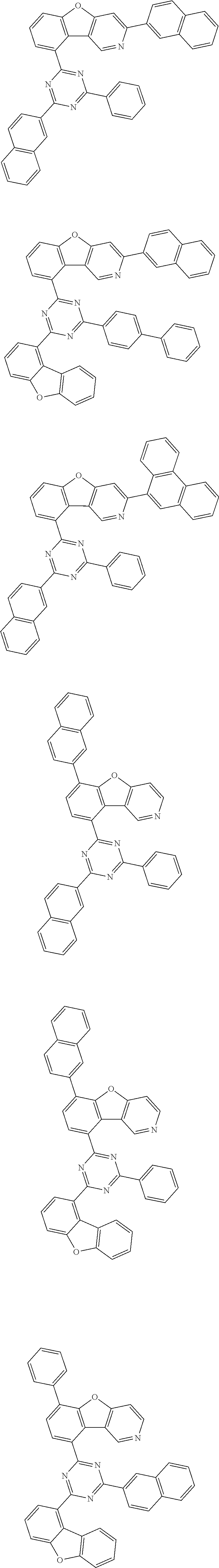 [SMILES: c1ccc(-c2nc(-c3ccc4ccccc4c3)nc(-c3cccc4oc5cc(-c6ccc7ccccc7c6)ncc5c34)n2)cc1 ; c1ccc(-c2ccc(-c3nc(-c4cccc5oc6ccccc6c45)nc(-c4cccc5oc6cc(-c7ccc8ccccc8c7)ncc6c45)n3)cc2)cc1 ; c1ccc(-c2nc(-c3ccc4ccccc4c3)nc(-c3cccc4oc5cc(-c6cc7ccccc7c7ccccc67)ncc5c34)n2)cc1 ; c1ccc(-c2nc(-c3ccc4ccccc4c3)nc(-c3ccc(-c4ccc5ccccc5c4)c4oc5ccncc5c34)n2)cc1 ; c1ccc(-c2nc(-c3cccc4oc5ccccc5c34)nc(-c3ccc(-c4ccc5ccccc5c4)c4oc5ccncc5c34)n2)cc1 ; c1ccc(-c2ccc(-c3nc(-c4ccc5ccccc5c4)nc(-c4cccc5oc6ccccc6c45)n3)c3c2oc2ccncc23)cc1]

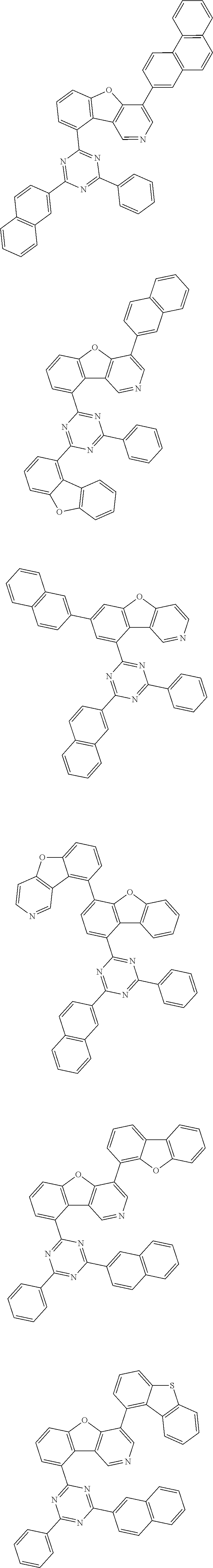 [SMILES: c1ccc(-c2nc(-c3ccc4ccccc4c3)nc(-c3cccc4oc5c(-c6ccc7c(ccc8ccccc87)c6)cncc5c34)n2)cc1 ; c1ccc(-c2nc(-c3cccc4oc5ccccc5c34)nc(-c3cccc4oc5c(-c6ccc7ccccc7c6)cncc5c34)n2)cc1 ; c1ccc(-c2nc(-c3ccc4ccccc4c3)nc(-c3cc(-c4ccc5ccccc5c4)cc4oc5ccncc5c34)n2)cc1 ; c1ccc(-c2nc(-c3ccc4ccccc4c3)nc(-c3ccc(-c4cccc5oc6ccncc6c45)c4oc5ccccc5c34)n2)cc1 ; c1ccc(-c2nc(-c3ccc4ccccc4c3)nc(-c3cccc4oc5c(-c6cccc7c6oc6ccccc67)cncc5c34)n2)cc1 ; c1ccc(-c2nc(-c3ccc4ccccc4c3)nc(-c3cccc4oc5c(-c6cccc7sc8ccccc8c67)cncc5c34)n2)cc1]

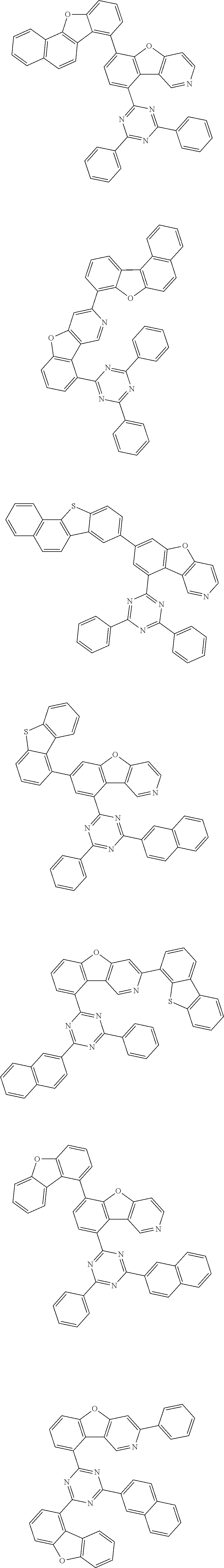

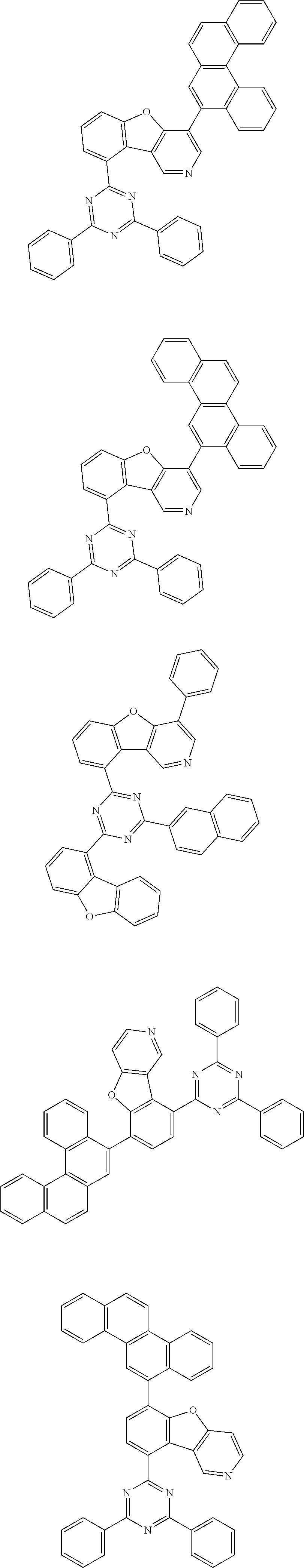 [SMILES: c1ccc(-c2nc(-c3ccccc3)nc(-c3cccc4oc5c(-c6cc7ccc8ccccc8c7c7ccccc67)cncc5c34)n2)cc1 ; c1ccc(-c2nc(-c3ccccc3)nc(-c3cccc4oc5c(-c6cc7c8ccccc8ccc7c7ccccc67)cncc5c34)n2)cc1 ; c1ccc(-c2cncc3c2oc2cccc(-c4nc(-c5ccc6ccccc6c5)nc(-c5cccc6oc7ccccc7c56)n4)c23)cc1 ; c1ccc(-c2nc(-c3ccccc3)nc(-c3ccc(-c4cc5ccc6ccccc6c5c5ccccc45)c4oc5ccncc5c34)n2)cc1 ; c1ccc(-c2nc(-c3ccccc3)nc(-c3ccc(-c4cc5c6ccccc6ccc5c5ccccc45)c4oc5ccncc5c34)n2)cc1]

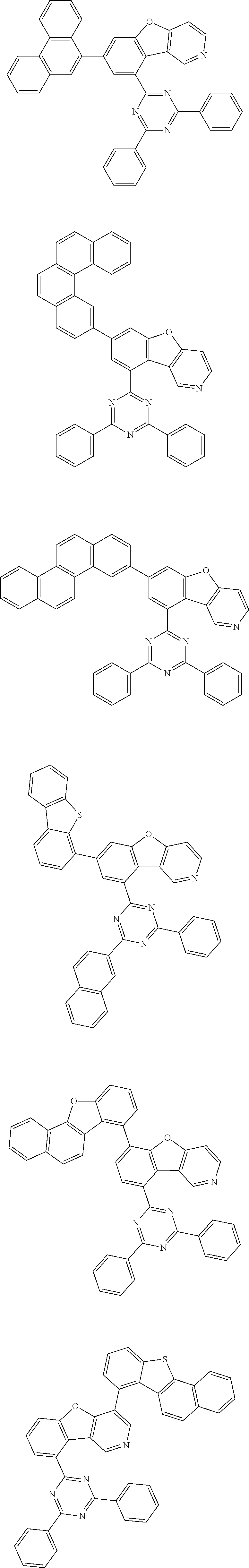 [SMILES: c1ccc(-c2nc(-c3ccccc3)nc(-c3cc(-c4cc5ccccc5c5ccccc45)cc4oc5ccncc5c34)n2)cc1 ; c1ccc(-c2nc(-c3ccccc3)nc(-c3cc(-c4ccc5ccc6ccc7ccccc7c6c5c4)cc4oc5ccncc5c34)n2)cc1 ; c1ccc(-c2nc(-c3ccccc3)nc(-c3cc(-c4ccc5ccc6c7ccccc7ccc6c5c4)cc4oc5ccncc5c34)n2)cc1 ; c1ccc(-c2nc(-c3ccc4ccccc4c3)nc(-c3cc(-c4cccc5c4sc4ccccc45)cc4oc5ccncc5c34)n2)cc1 ; c1ccc(-c2nc(-c3ccccc3)nc(-c3ccc(-c4cccc5oc6c7ccccc7ccc6c45)c4oc5ccncc5c34)n2)cc1 ; c1ccc(-c2nc(-c3ccccc3)nc(-c3cccc4oc5c(-c6cccc7sc8c9ccccc9ccc8c67)cncc5c34)n2)cc1]

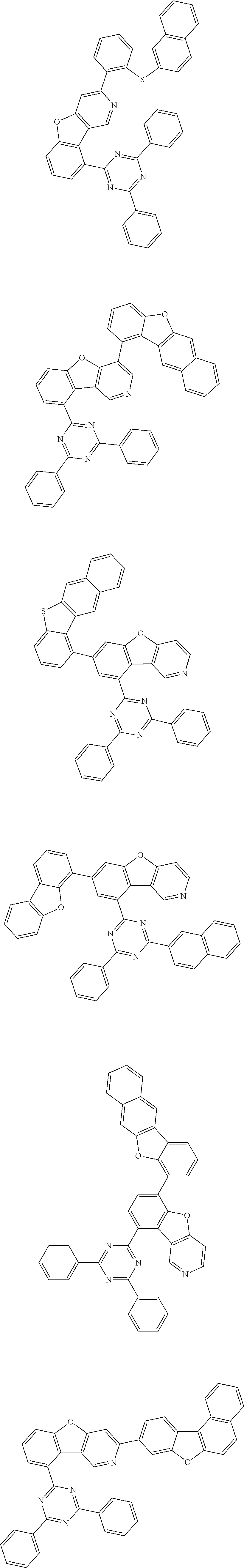 [SMILES: c1ccc(-c2nc(-c3ccccc3)nc(-c3cccc4oc5cc(-c6cccc7c6sc6ccc8ccccc8c67)ncc5c34)n2)cc1 ; c1ccc(-c2nc(-c3ccccc3)nc(-c3cccc4oc5c(-c6cccc7oc8cc9ccccc9cc8c67)cncc5c34)n2)cc1 ; c1ccc(-c2nc(-c3ccccc3)nc(-c3cc(-c4cccc5sc6cc7ccccc7cc6c45)cc4oc5ccncc5c34)n2)cc1 ; c1ccc(-c2nc(-c3ccc4ccccc4c3)nc(-c3cc(-c4cccc5c4oc4ccccc45)cc4oc5ccncc5c34)n2)cc1 ; c1ccc(-c2nc(-c3ccccc3)nc(-c3ccc(-c4cccc5c4oc4cc6ccccc6cc45)c4oc5ccncc5c34)n2)cc1 ; c1ccc(-c2nc(-c3ccccc3)nc(-c3cccc4oc5cc(-c6ccc7c(c6)oc6ccc8ccccc8c67)ncc5c34)n2)cc1]

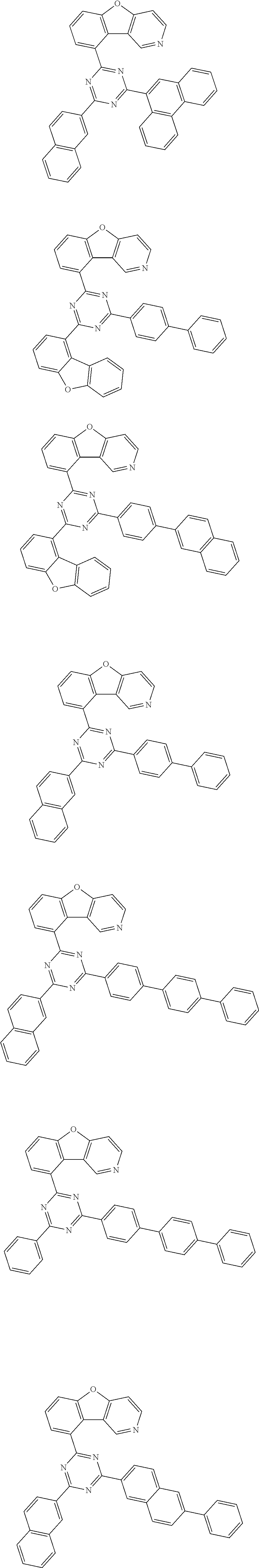

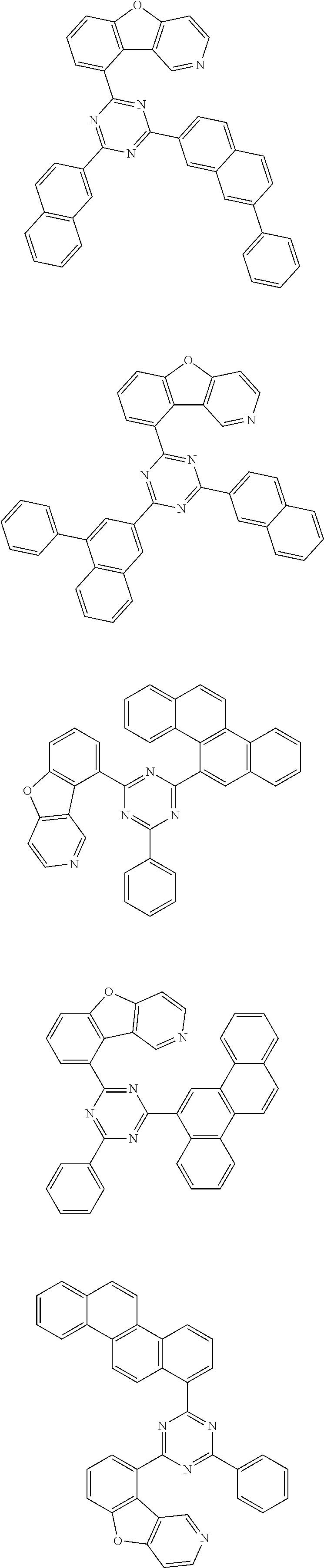 [SMILES: c1ccc(-c2ccc3ccc(-c4nc(-c5ccc6ccccc6c5)nc(-c5cccc6oc7ccncc7c56)n4)cc3c2)cc1 ; c1ccc(-c2cc(-c3nc(-c4ccc5ccccc5c4)nc(-c4cccc5oc6ccncc6c45)n3)cc3ccccc23)cc1 ; c1ccc(-c2nc(-c3cccc4oc5ccncc5c34)nc(-c3cc4ccccc4c4ccc5ccccc5c34)n2)cc1 ; c1ccc(-c2nc(-c3cc4c5ccccc5ccc4c4ccccc34)nc(-c3cccc4oc5ccncc5c34)n2)cc1 ; c1ccc(-c2nc(-c3cccc4c3ccc3c5ccccc5ccc43)nc(-c3cccc4oc5ccncc5c34)n2)cc1]

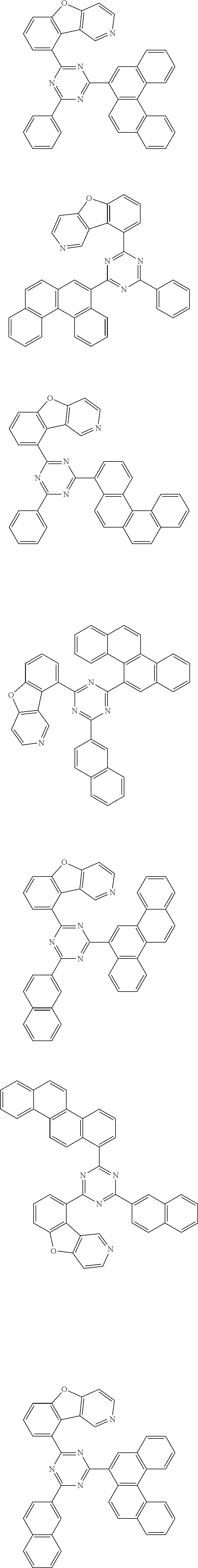

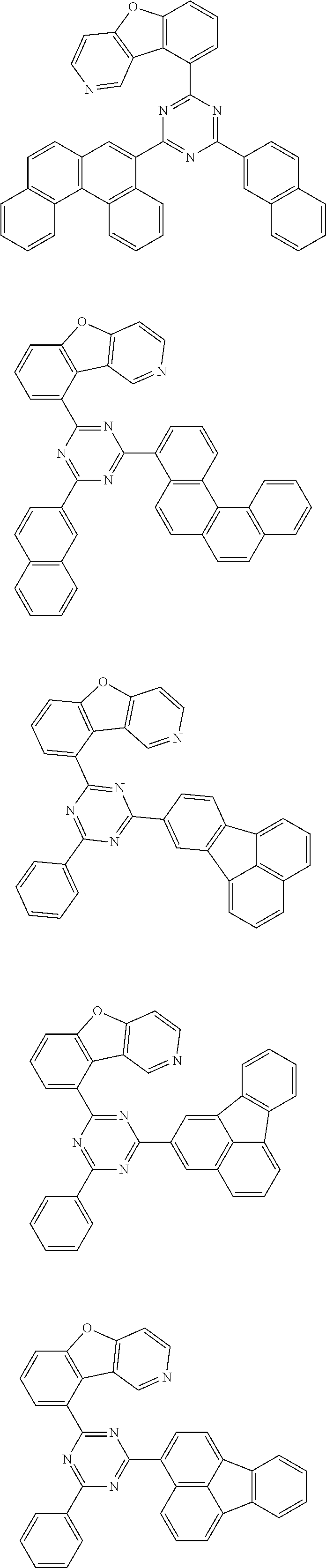 [SMILES: c1ccc2cc(-c3nc(-c4cc5ccc6ccccc6c5c5ccccc45)nc(-c4cccc5oc6ccncc6c45)n3)ccc2c1 ; c1ccc2cc(-c3nc(-c4cccc5c4ccc4ccc6ccccc6c45)nc(-c4cccc5oc6ccncc6c45)n3)ccc2c1 ; c1ccc(-c2nc(-c3ccc4c(c3)-c3cccc5cccc-4c35)nc(-c3cccc4oc5ccncc5c34)n2)cc1 ; c1ccc(-c2nc(-c3cc4c5c(cccc5c3)-c3ccccc3-4)nc(-c3cccc4oc5ccncc5c34)n2)cc1 ; c1ccc(-c2nc(-c3ccc4c5c(cccc35)-c3ccccc3-4)nc(-c3cccc4oc5ccncc5c34)n2)cc1]

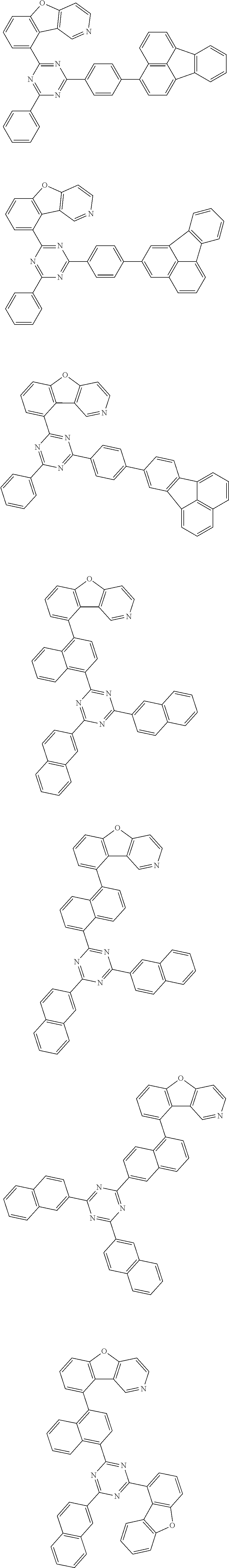

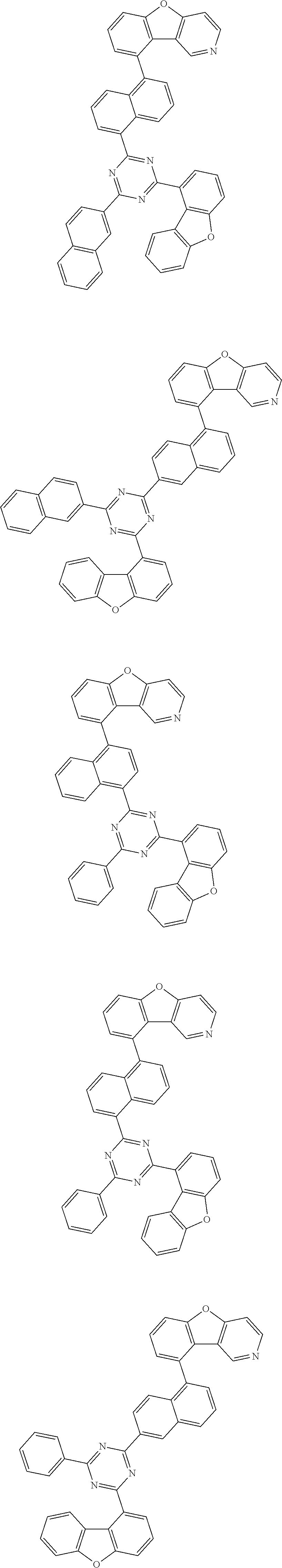 [SMILES: c1ccc2cc(-c3nc(-c4cccc5c(-c6cccc7oc8ccncc8c67)cccc45)nc(-c4cccc5oc6ccccc6c45)n3)ccc2c1 ; c1ccc2cc(-c3nc(-c4ccc5c(-c6cccc7oc8ccncc8c67)cccc5c4)nc(-c4cccc5oc6ccccc6c45)n3)ccc2c1 ; c1ccc(-c2nc(-c3ccc(-c4cccc5oc6ccncc6c45)c4ccccc34)nc(-c3cccc4oc5ccccc5c34)n2)cc1 ; c1ccc(-c2nc(-c3cccc4c(-c5cccc6oc7ccncc7c56)cccc34)nc(-c3cccc4oc5ccccc5c34)n2)cc1 ; c1ccc(-c2nc(-c3ccc4c(-c5cccc6oc7ccncc7c56)cccc4c3)nc(-c3cccc4oc5ccccc5c34)n2)cc1]

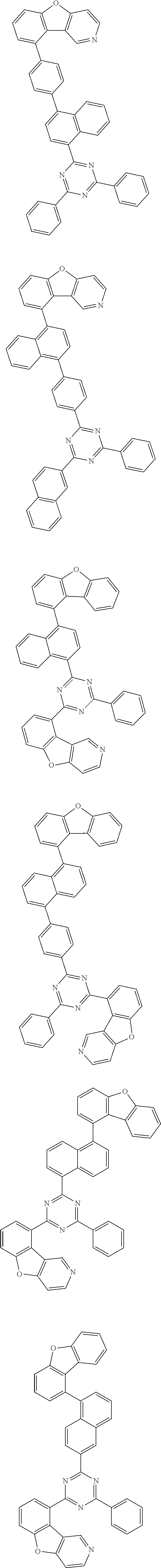

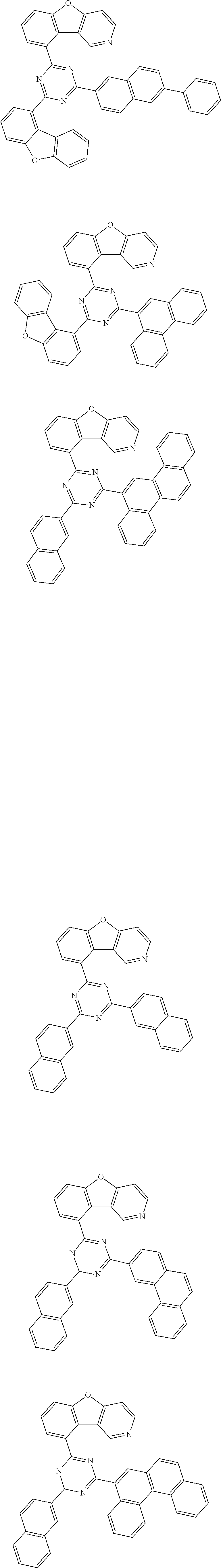

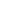

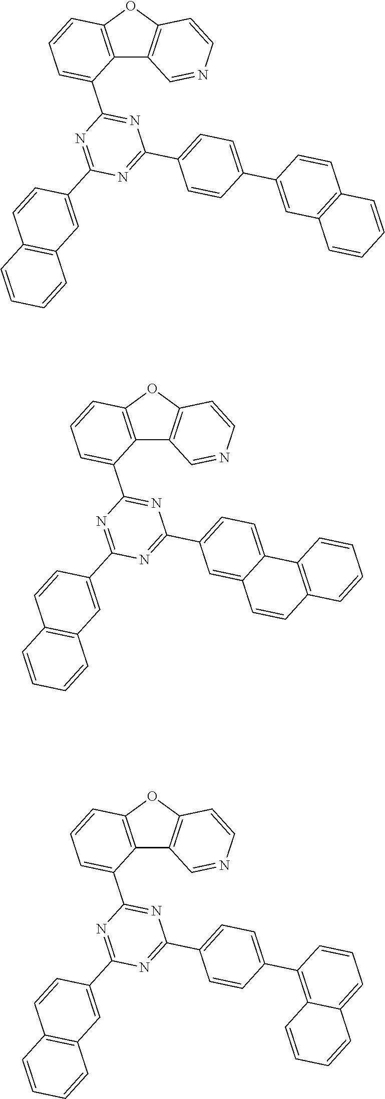 [SMILES: c1ccc2cc(-c3ccc(-c4nc(-c5ccc6ccccc6c5)nc(-c5cccc6oc7ccncc7c56)n4)cc3)ccc2c1 ; c1ccc2cc(-c3nc(-c4ccc5c(ccc6ccccc65)c4)nc(-c4cccc5oc6ccncc6c45)n3)ccc2c1 ; c1ccc2cc(-c3nc(-c4ccc(-c5cccc6ccccc56)cc4)nc(-c4cccc5oc6ccncc6c45)n3)ccc2c1]

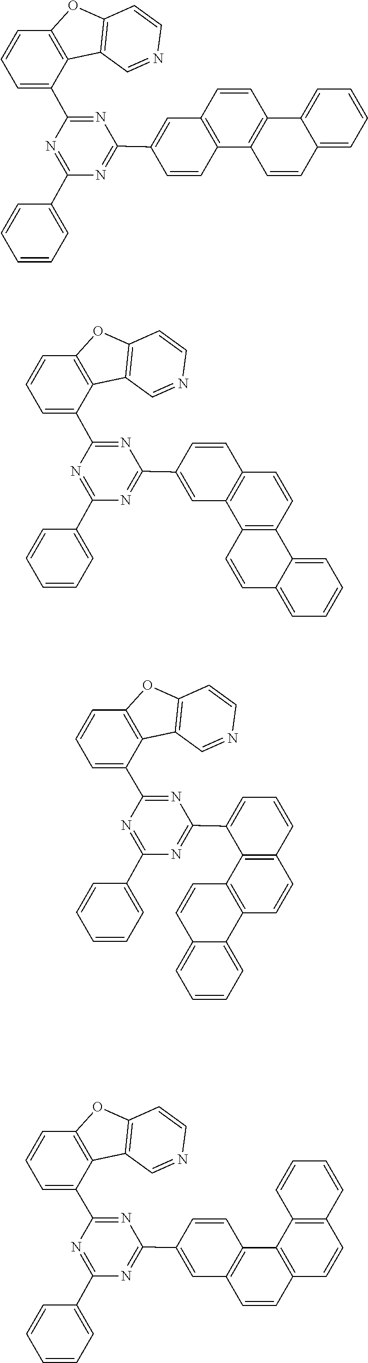 [SMILES: c1ccc(-c2nc(-c3ccc4c(ccc5c6ccccc6ccc45)c3)nc(-c3cccc4oc5ccncc5c34)n2)cc1 ; c1ccc(-c2nc(-c3ccc4ccc5c6ccccc6ccc5c4c3)nc(-c3cccc4oc5ccncc5c34)n2)cc1 ; c1ccc(-c2nc(-c3cccc4ccc5c6ccccc6ccc5c34)nc(-c3cccc4oc5ccncc5c34)n2)cc1 ; c1ccc(-c2nc(-c3ccc4c(ccc5ccc6ccccc6c54)c3)nc(-c3cccc4oc5ccncc5c34)n2)cc1]

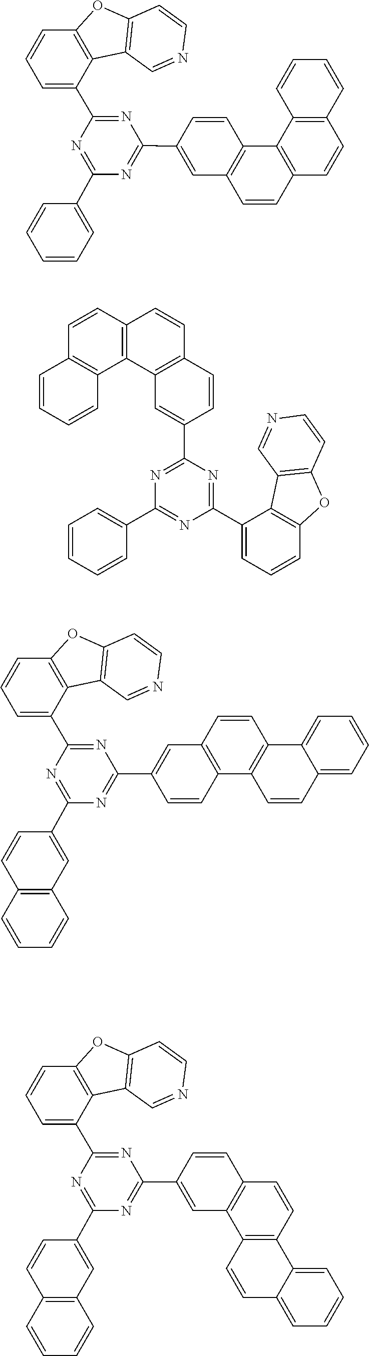 [SMILES: c1ccc(-c2nc(-c3ccc4c(ccc5ccc6ccccc6c54)c3)nc(-c3cccc4oc5ccncc5c34)n2)cc1 ; c1ccc(-c2nc(-c3ccc4ccc5ccc6ccccc6c5c4c3)nc(-c3cccc4oc5ccncc5c34)n2)cc1 ; c1ccc2cc(-c3nc(-c4ccc5c(ccc6c7ccccc7ccc56)c4)nc(-c4cccc5oc6ccncc6c45)n3)ccc2c1 ; c1ccc2cc(-c3nc(-c4ccc5ccc6c7ccccc7ccc6c5c4)nc(-c4cccc5oc6ccncc6c45)n3)ccc2c1]

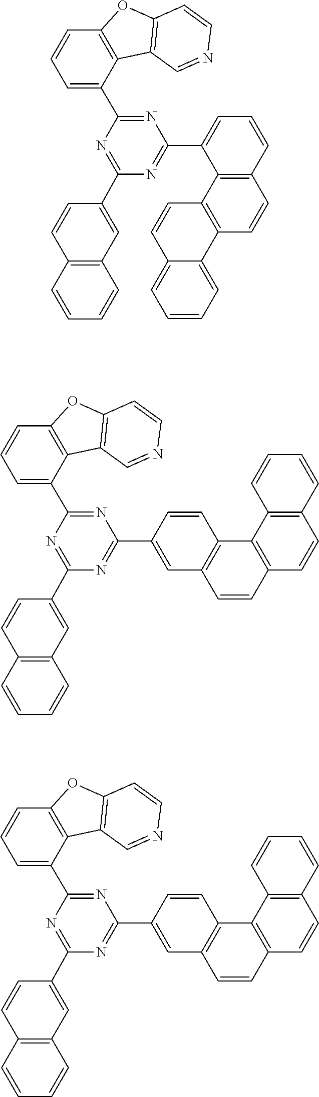 [SMILES: c1ccc2cc(-c3nc(-c4cccc5ccc6c7ccccc7ccc6c45)nc(-c4cccc5oc6ccncc6c45)n3)ccc2c1 ; c1ccc2cc(-c3nc(-c4ccc5c(ccc6ccc7ccccc7c65)c4)nc(-c4cccc5oc6ccncc6c45)n3)ccc2c1 ; c1ccc2cc(-c3nc(-c4ccc5c(ccc6ccc7ccccc7c65)c4)nc(-c4cccc5oc6ccncc6c45)n3)ccc2c1]

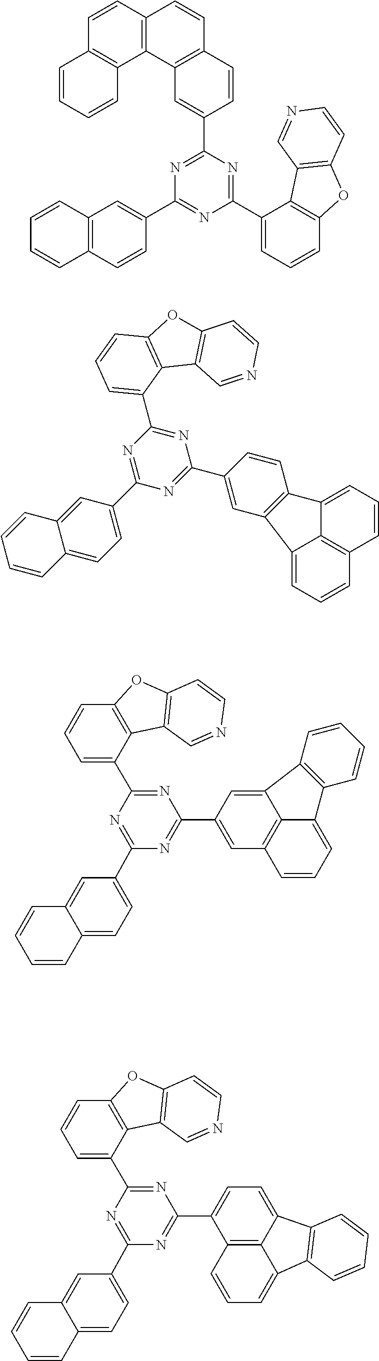 [SMILES: c1ccc2cc(-c3nc(-c4ccc5ccc6ccc7ccccc7c6c5c4)nc(-c4cccc5oc6ccncc6c45)n3)ccc2c1 ; c1ccc2cc(-c3nc(-c4ccc5c(c4)-c4cccc6cccc-5c46)nc(-c4cccc5oc6ccncc6c45)n3)ccc2c1 ; c1ccc2c(c1)-c1cccc3cc(-c4nc(-c5ccc6ccccc6c5)nc(-c5cccc6oc7ccncc7c56)n4)cc-2c13 ; c1ccc2c(c1)-c1cccc3c(-c4nc(-c5ccc6ccccc6c5)nc(-c5cccc6oc7ccncc7c56)n4)ccc-2c13]

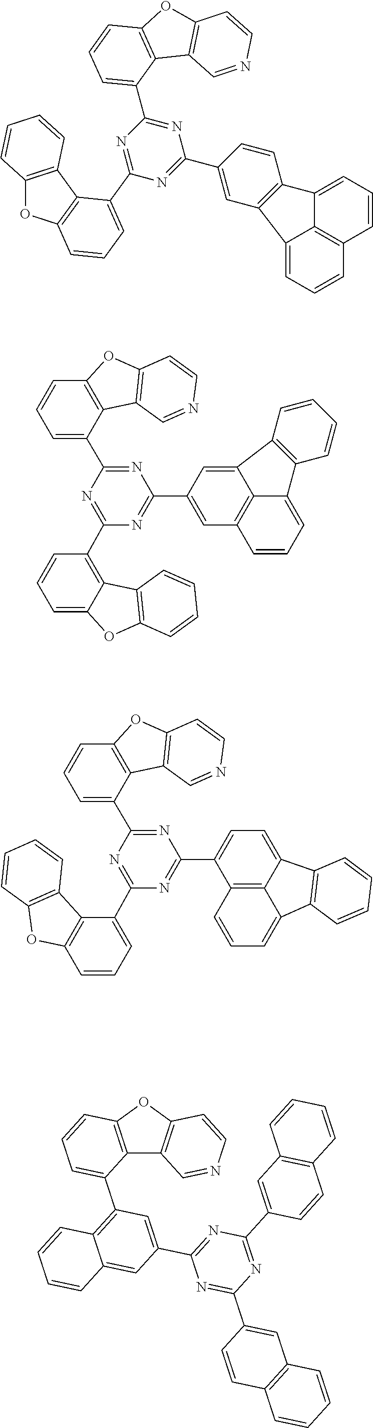 [SMILES: c1cc2c3c(cccc3c1)-c1cc(-c3nc(-c4cccc5oc6ccccc6c45)nc(-c4cccc5oc6ccncc6c45)n3)ccc1-2 ; c1ccc2c(c1)-c1cccc3cc(-c4nc(-c5cccc6oc7ccccc7c56)nc(-c5cccc6oc7ccncc7c56)n4)cc-2c13 ; c1ccc2c(c1)-c1cccc3c(-c4nc(-c5cccc6oc7ccccc7c56)nc(-c5cccc6oc7ccncc7c56)n4)ccc-2c13 ; c1ccc2cc(-c3nc(-c4ccc5ccccc5c4)nc(-c4cc(-c5cccc6oc7ccncc7c56)c5ccccc5c4)n3)ccc2c1]

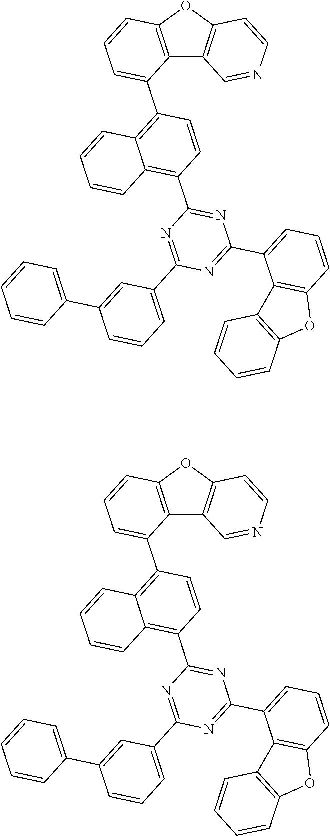 [SMILES: c1ccc(-c2cccc(-c3nc(-c4ccc(-c5cccc6oc7ccncc7c56)c5ccccc45)nc(-c4cccc5oc6ccccc6c45)n3)c2)cc1 ; c1ccc(-c2cccc(-c3nc(-c4ccc(-c5cccc6oc7ccncc7c56)c5ccccc45)nc(-c4cccc5oc6ccccc6c45)n3)c2)cc1]

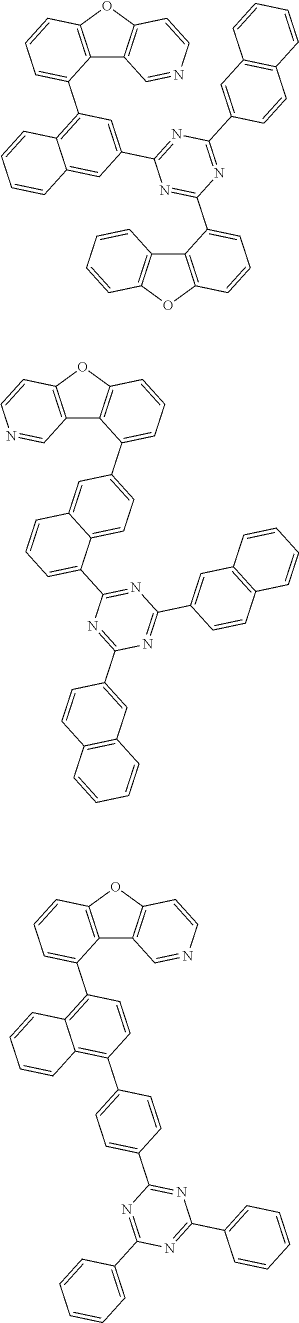 [SMILES: c1ccc2cc(-c3nc(-c4cc(-c5cccc6oc7ccncc7c56)c5ccccc5c4)nc(-c4cccc5oc6ccccc6c45)n3)ccc2c1 ; c1ccc2cc(-c3nc(-c4ccc5ccccc5c4)nc(-c4cccc5cc(-c6cccc7oc8ccncc8c67)ccc45)n3)ccc2c1 ; c1ccc(-c2nc(-c3ccccc3)nc(-c3ccc(-c4ccc(-c5cccc6oc7ccncc7c56)c5ccccc45)cc3)n2)cc1]

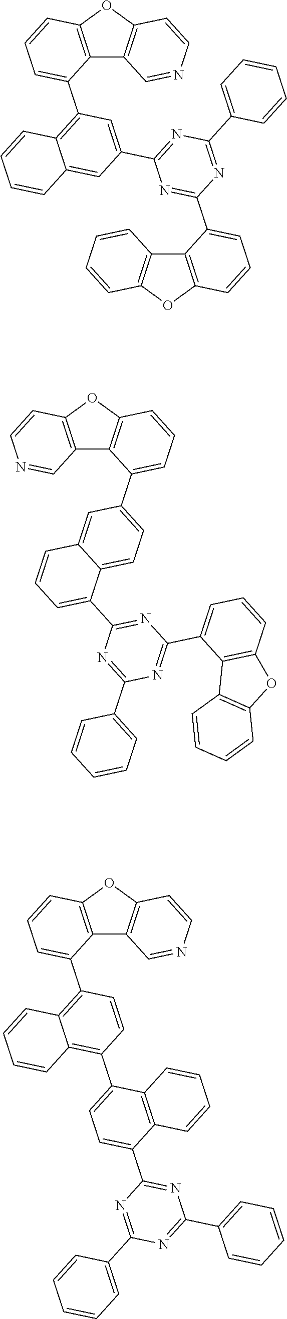 [SMILES: c1ccc(-c2nc(-c3cc(-c4cccc5oc6ccncc6c45)c4ccccc4c3)nc(-c3cccc4oc5ccccc5c34)n2)cc1 ; c1ccc(-c2nc(-c3cccc4cc(-c5cccc6oc7ccncc7c56)ccc34)nc(-c3cccc4oc5ccccc5c34)n2)cc1 ; c1ccc(-c2nc(-c3ccccc3)nc(-c3ccc(-c4ccc(-c5cccc6oc7ccncc7c56)c5ccccc45)c4ccccc34)n2)cc1]

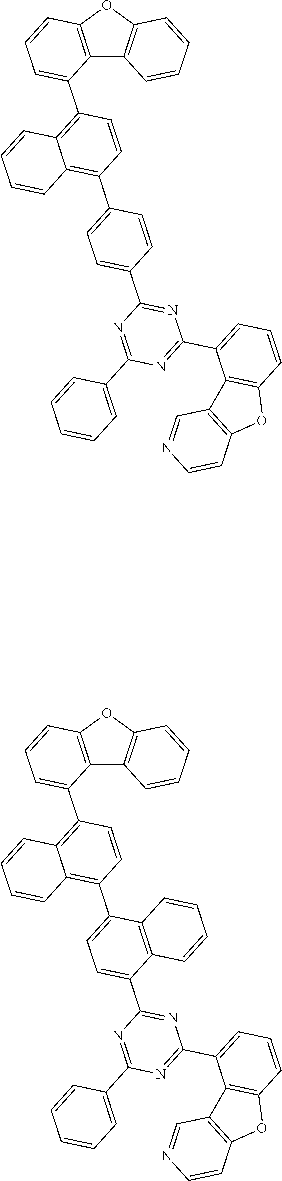 [SMILES: c1ccc(-c2nc(-c3ccc(-c4ccc(-c5cccc6oc7ccccc7c56)c5ccccc45)cc3)nc(-c3cccc4oc5ccncc5c34)n2)cc1 ; c1ccc(-c2nc(-c3ccc(-c4ccc(-c5cccc6oc7ccccc7c56)c5ccccc45)c4ccccc34)nc(-c3cccc4oc5ccncc5c34)n2)cc1]

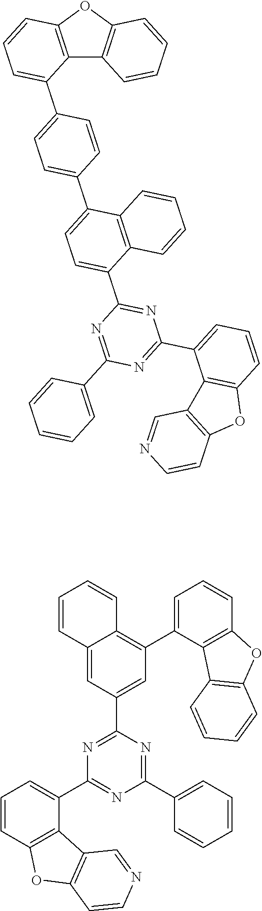 [SMILES: c1ccc(-c2nc(-c3ccc(-c4ccc(-c5cccc6oc7ccccc7c56)cc4)c4ccccc34)nc(-c3cccc4oc5ccncc5c34)n2)cc1 ; c1ccc(-c2nc(-c3cc(-c4cccc5oc6ccccc6c45)c4ccccc4c3)nc(-c3cccc4oc5ccncc5c34)n2)cc1]

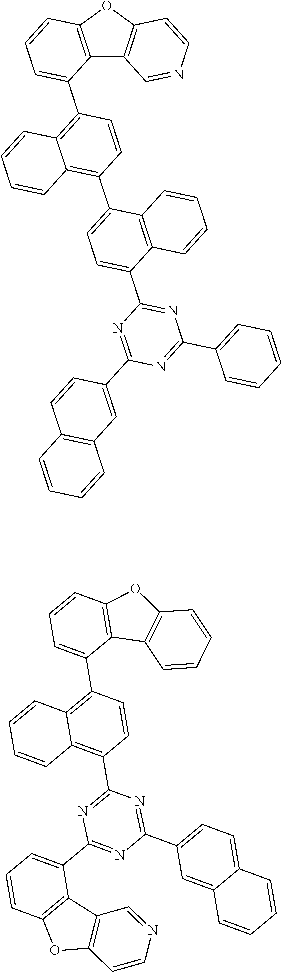 [SMILES: c1ccc(-c2nc(-c3ccc4ccccc4c3)nc(-c3ccc(-c4ccc(-c5cccc6oc7ccncc7c56)c5ccccc45)c4ccccc34)n2)cc1 ; c1ccc2cc(-c3nc(-c4ccc(-c5cccc6oc7ccccc7c56)c5ccccc45)nc(-c4cccc5oc6ccncc6c45)n3)ccc2c1]

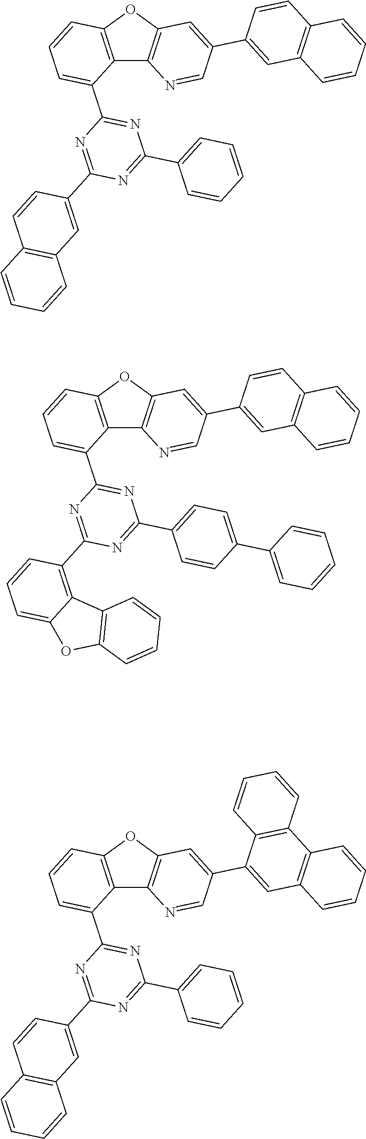 [SMILES: c1ccc(-c2nc(-c3ccc4ccccc4c3)nc(-c3cccc4oc5cc(-c6ccc7ccccc7c6)cnc5c34)n2)cc1 ; c1ccc(-c2ccc(-c3nc(-c4cccc5oc6ccccc6c45)nc(-c4cccc5oc6cc(-c7ccc8ccccc8c7)cnc6c45)n3)cc2)cc1 ; c1ccc(-c2nc(-c3ccc4ccccc4c3)nc(-c3cccc4oc5cc(-c6cc7ccccc7c7ccccc67)cnc5c34)n2)cc1]

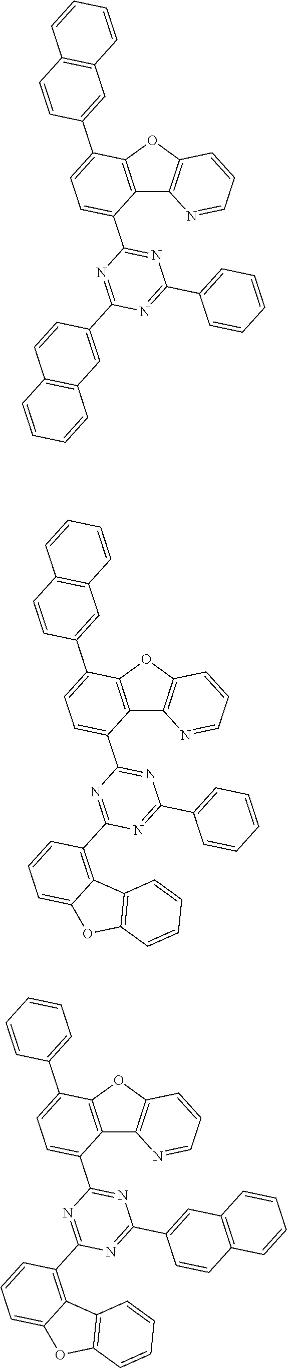 [SMILES: c1ccc(-c2nc(-c3ccc4ccccc4c3)nc(-c3ccc(-c4ccc5ccccc5c4)c4oc5cccnc5c34)n2)cc1 ; c1ccc(-c2nc(-c3cccc4oc5ccccc5c34)nc(-c3ccc(-c4ccc5ccccc5c4)c4oc5cccnc5c34)n2)cc1 ; c1ccc(-c2ccc(-c3nc(-c4ccc5ccccc5c4)nc(-c4cccc5oc6ccccc6c45)n3)c3c2oc2cccnc23)cc1]

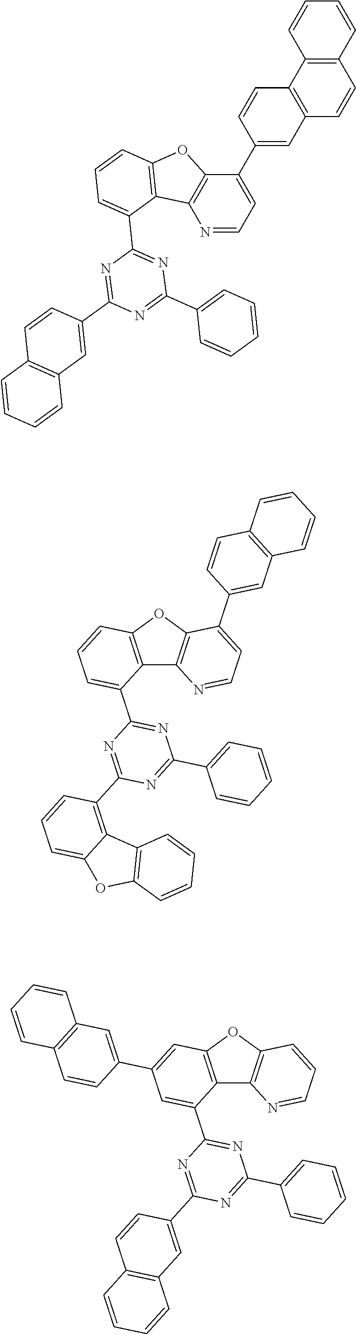 [SMILES: c1ccc(-c2nc(-c3ccc4ccccc4c3)nc(-c3cccc4oc5c(-c6ccc7c(ccc8ccccc87)c6)ccnc5c34)n2)cc1 ; c1ccc(-c2nc(-c3cccc4oc5ccccc5c34)nc(-c3cccc4oc5c(-c6ccc7ccccc7c6)ccnc5c34)n2)cc1 ; c1ccc(-c2nc(-c3ccc4ccccc4c3)nc(-c3cc(-c4ccc5ccccc5c4)cc4oc5cccnc5c34)n2)cc1]

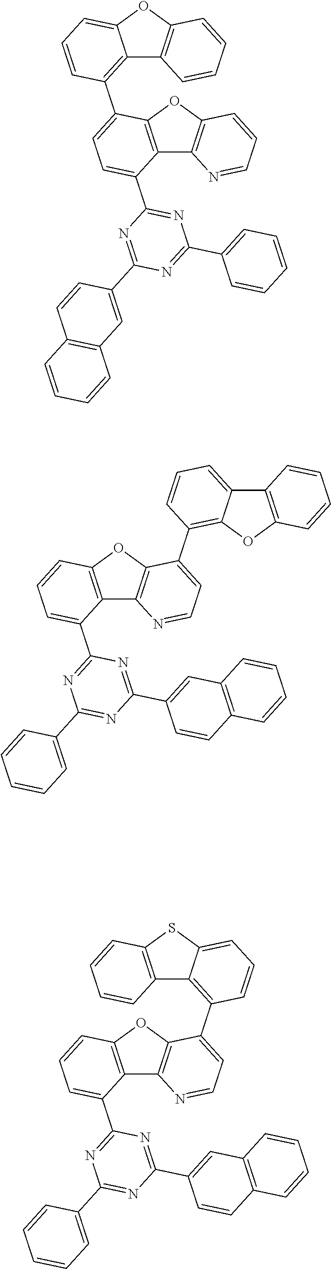 [SMILES: c1ccc(-c2nc(-c3ccc4ccccc4c3)nc(-c3ccc(-c4cccc5oc6ccccc6c45)c4oc5cccnc5c34)n2)cc1 ; c1ccc(-c2nc(-c3ccc4ccccc4c3)nc(-c3cccc4oc5c(-c6cccc7c6oc6ccccc67)ccnc5c34)n2)cc1 ; c1ccc(-c2nc(-c3ccc4ccccc4c3)nc(-c3cccc4oc5c(-c6cccc7sc8ccccc8c67)ccnc5c34)n2)cc1]

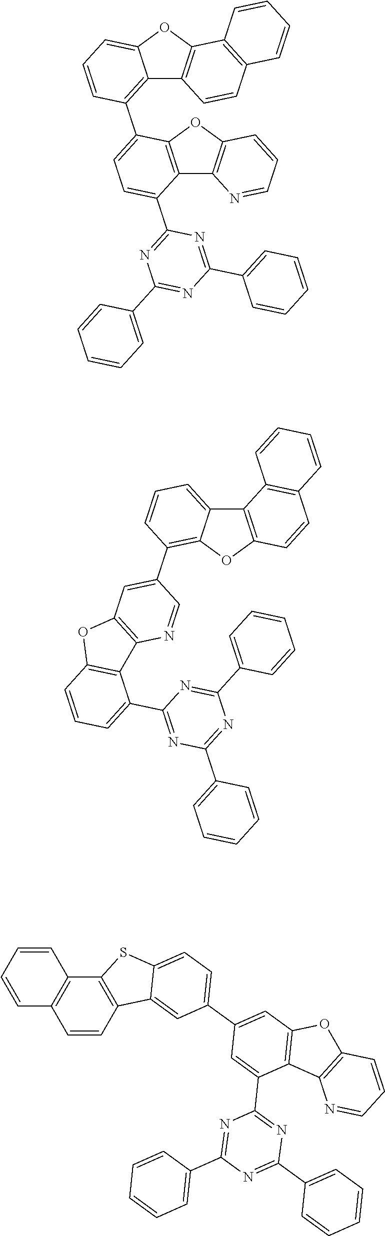 [SMILES: c1ccc(-c2nc(-c3ccccc3)nc(-c3ccc(-c4cccc5oc6c7ccccc7ccc6c45)c4oc5cccnc5c34)n2)cc1 ; c1ccc(-c2nc(-c3ccccc3)nc(-c3cccc4oc5cc(-c6cccc7c6oc6ccc8ccccc8c67)cnc5c34)n2)cc1 ; c1ccc(-c2nc(-c3ccccc3)nc(-c3cc(-c4ccc5sc6c7ccccc7ccc6c5c4)cc4oc5cccnc5c34)n2)cc1]

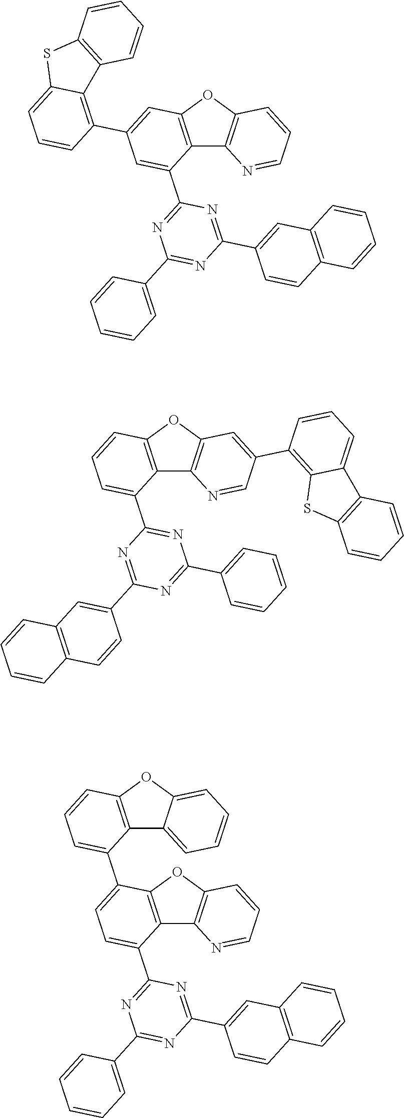 [SMILES: c1ccc(-c2nc(-c3ccc4ccccc4c3)nc(-c3cc(-c4cccc5sc6ccccc6c45)cc4oc5cccnc5c34)n2)cc1 ; c1ccc(-c2nc(-c3ccc4ccccc4c3)nc(-c3cccc4oc5cc(-c6cccc7c6sc6ccccc67)cnc5c34)n2)cc1 ; c1ccc(-c2nc(-c3ccc4ccccc4c3)nc(-c3ccc(-c4cccc5oc6ccccc6c45)c4oc5cccnc5c34)n2)cc1]

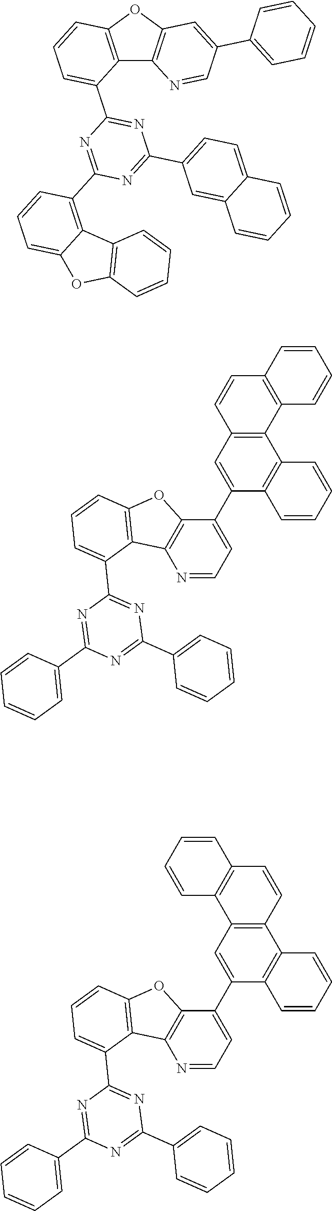 [SMILES: c1ccc(-c2cnc3c(c2)oc2cccc(-c4nc(-c5ccc6ccccc6c5)nc(-c5cccc6oc7ccccc7c56)n4)c23)cc1 ; c1ccc(-c2nc(-c3ccccc3)nc(-c3cccc4oc5c(-c6cc7ccc8ccccc8c7c7ccccc67)ccnc5c34)n2)cc1 ; c1ccc(-c2nc(-c3ccccc3)nc(-c3cccc4oc5c(-c6cc7c8ccccc8ccc7c7ccccc67)ccnc5c34)n2)cc1]

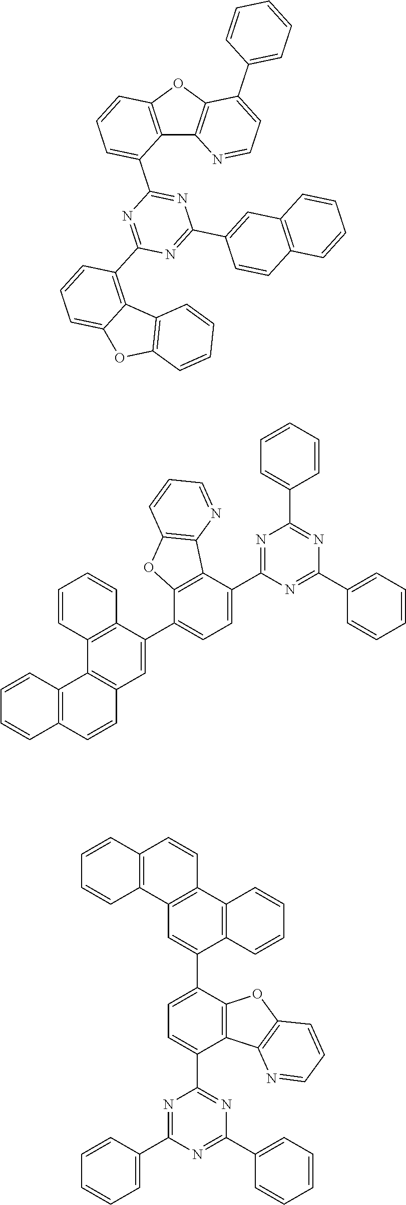 [SMILES: c1ccc(-c2ccnc3c2oc2cccc(-c4nc(-c5ccc6ccccc6c5)nc(-c5cccc6oc7ccccc7c56)n4)c23)cc1 ; c1ccc(-c2nc(-c3ccccc3)nc(-c3ccc(-c4cc5ccc6ccccc6c5c5ccccc45)c4oc5cccnc5c34)n2)cc1 ; c1ccc(-c2nc(-c3ccccc3)nc(-c3ccc(-c4cc5c6ccccc6ccc5c5ccccc45)c4oc5cccnc5c34)n2)cc1]

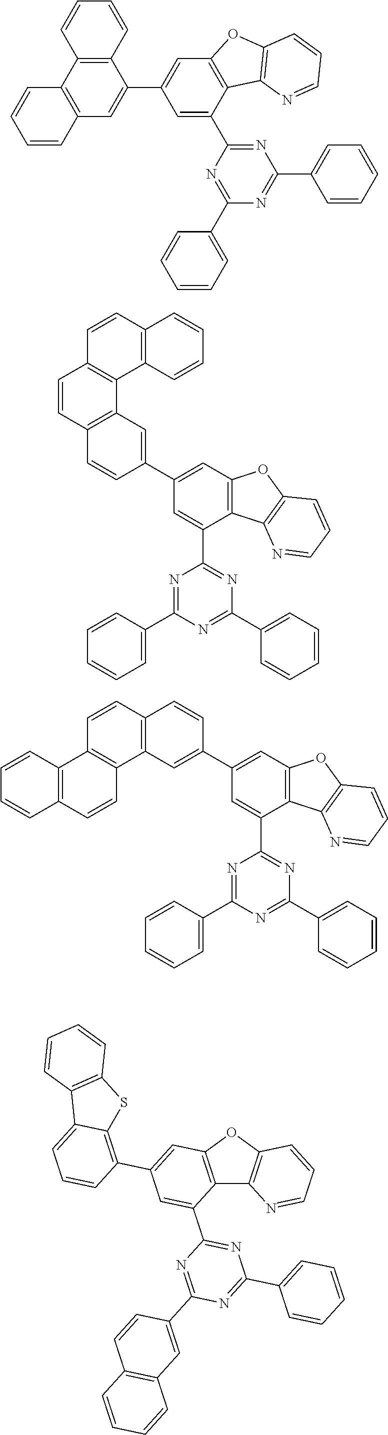

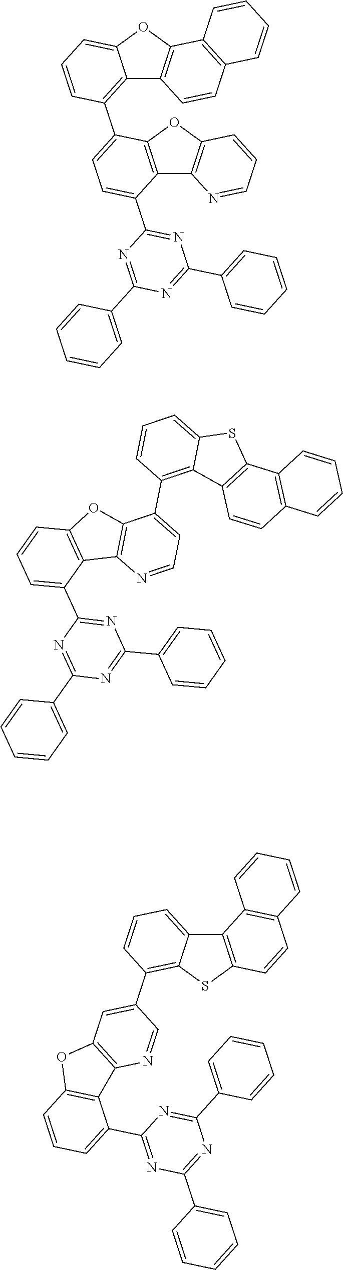 [SMILES: c1ccc(-c2nc(-c3ccccc3)nc(-c3ccc(-c4cccc5oc6c7ccccc7ccc6c45)c4oc5cccnc5c34)n2)cc1 ; c1ccc(-c2nc(-c3ccccc3)nc(-c3cccc4oc5c(-c6cccc7sc8c9ccccc9ccc8c67)ccnc5c34)n2)cc1 ; c1ccc(-c2nc(-c3ccccc3)nc(-c3cccc4oc5cc(-c6cccc7c6sc6ccc8ccccc8c67)cnc5c34)n2)cc1]

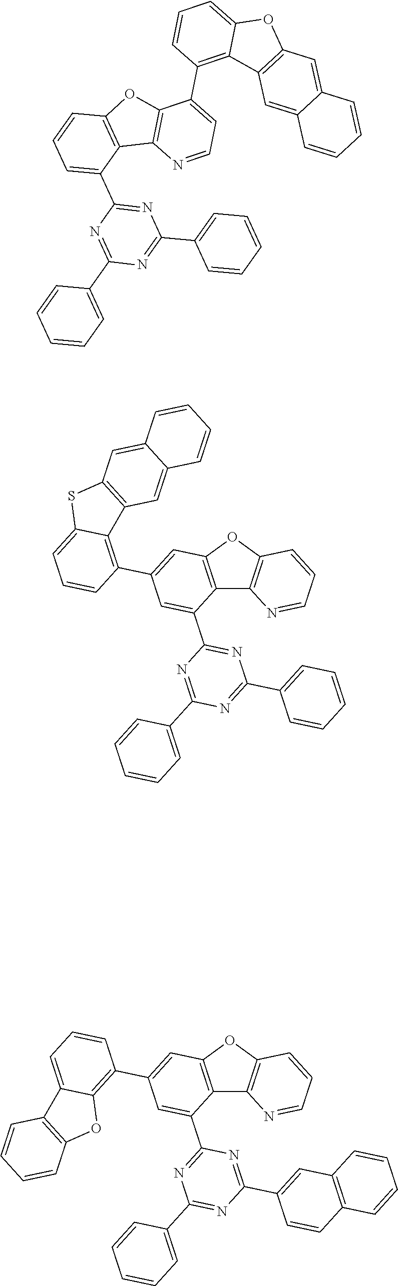 [SMILES: c1ccc(-c2nc(-c3ccccc3)nc(-c3cccc4oc5c(-c6cccc7oc8cc9ccccc9cc8c67)ccnc5c34)n2)cc1 ; c1ccc(-c2nc(-c3ccccc3)nc(-c3cc(-c4cccc5sc6cc7ccccc7cc6c45)cc4oc5cccnc5c34)n2)cc1 ; c1ccc(-c2nc(-c3ccc4ccccc4c3)nc(-c3cc(-c4cccc5c4oc4ccccc45)cc4oc5cccnc5c34)n2)cc1]

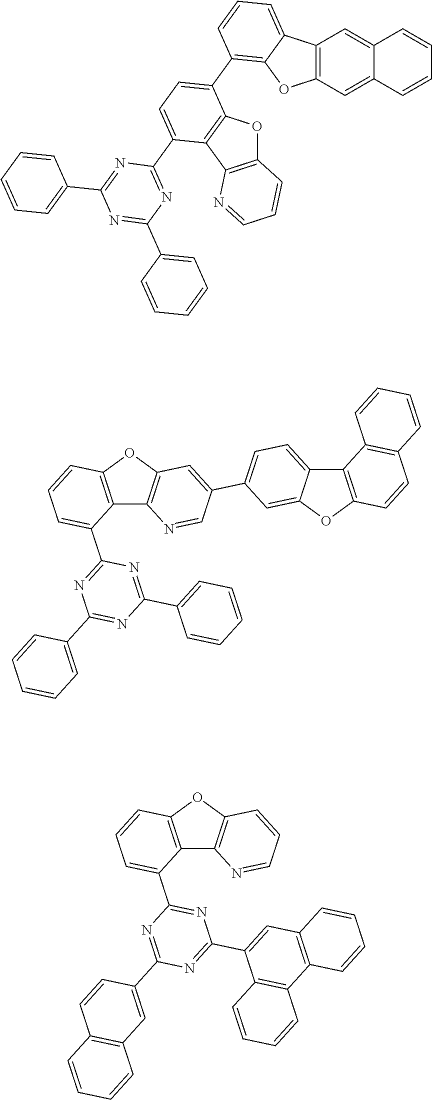 [SMILES: c1ccc(-c2nc(-c3ccccc3)nc(-c3ccc(-c4cccc5c4oc4cc6ccccc6cc45)c4oc5cccnc5c34)n2)cc1 ; c1ccc(-c2nc(-c3ccccc3)nc(-c3cccc4oc5cc(-c6ccc7c(c6)oc6ccc8ccccc8c67)cnc5c34)n2)cc1 ; c1ccc2cc(-c3nc(-c4cc5ccccc5c5ccccc45)nc(-c4cccc5oc6cccnc6c45)n3)ccc2c1]

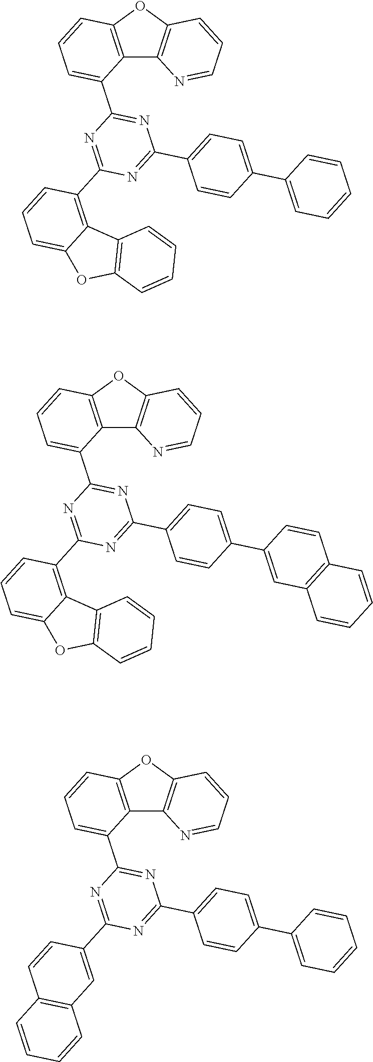 [SMILES: c1ccc(-c2ccc(-c3nc(-c4cccc5oc6ccccc6c45)nc(-c4cccc5oc6cccnc6c45)n3)cc2)cc1 ; c1ccc2cc(-c3ccc(-c4nc(-c5cccc6oc7ccccc7c56)nc(-c5cccc6oc7cccnc7c56)n4)cc3)ccc2c1 ; c1ccc(-c2ccc(-c3nc(-c4ccc5ccccc5c4)nc(-c4cccc5oc6cccnc6c45)n3)cc2)cc1]

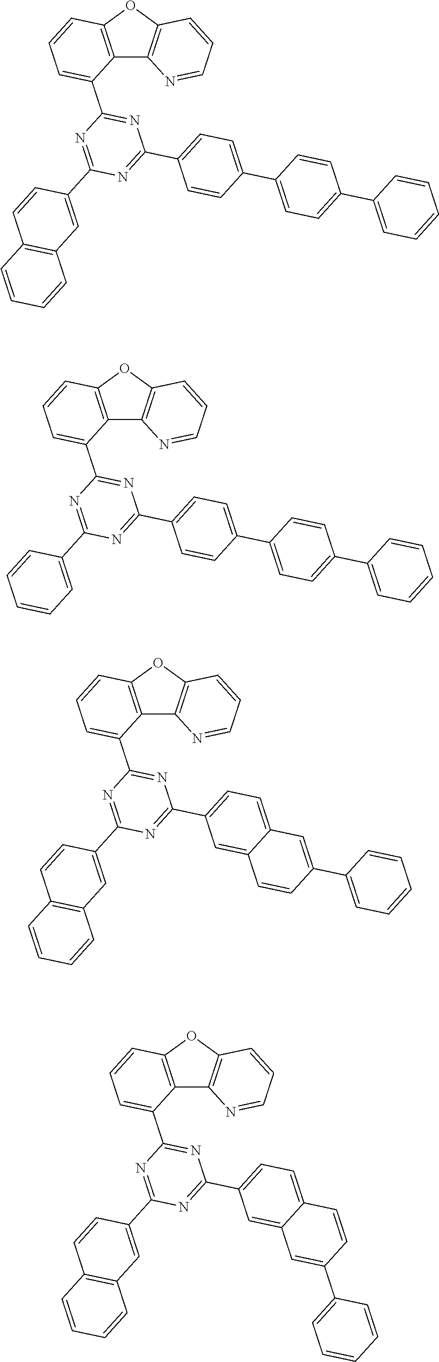 [SMILES: c1ccc(-c2ccc(-c3ccc(-c4nc(-c5ccc6ccccc6c5)nc(-c5cccc6oc7cccnc7c56)n4)cc3)cc2)cc1 ; c1ccc(-c2ccc(-c3ccc(-c4nc(-c5ccccc5)nc(-c5cccc6oc7cccnc7c56)n4)cc3)cc2)cc1 ; c1ccc(-c2ccc3cc(-c4nc(-c5ccc6ccccc6c5)nc(-c5cccc6oc7cccnc7c56)n4)ccc3c2)cc1 ; c1ccc(-c2ccc3ccc(-c4nc(-c5ccc6ccccc6c5)nc(-c5cccc6oc7cccnc7c56)n4)cc3c2)cc1]

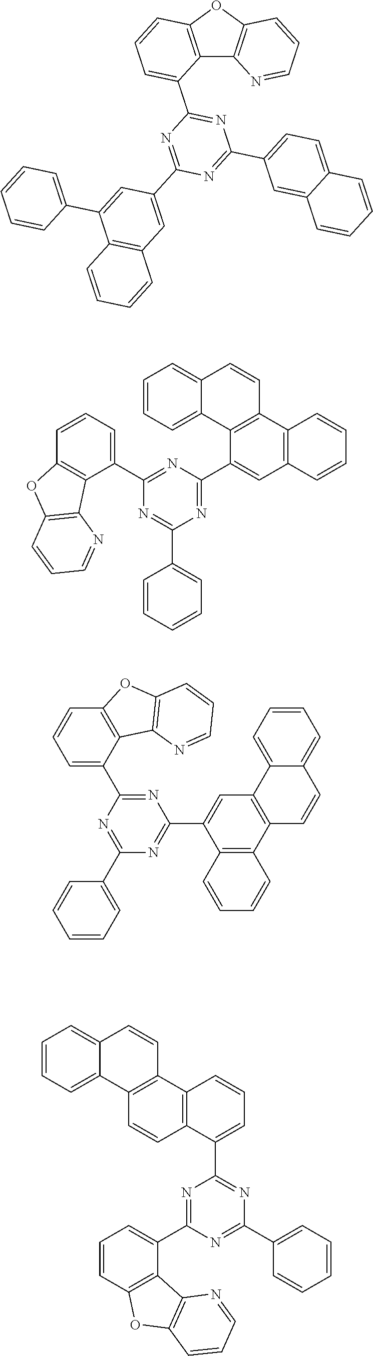 [SMILES: c1ccc(-c2cc(-c3nc(-c4ccc5ccccc5c4)nc(-c4cccc5oc6cccnc6c45)n3)cc3ccccc23)cc1 ; c1ccc(-c2nc(-c3cc4ccccc4c4ccc5ccccc5c34)nc(-c3cccc4oc5cccnc5c34)n2)cc1 ; c1ccc(-c2nc(-c3cc4c5ccccc5ccc4c4ccccc34)nc(-c3cccc4oc5cccnc5c34)n2)cc1 ; c1ccc(-c2nc(-c3cccc4c3ccc3c5ccccc5ccc43)nc(-c3cccc4oc5cccnc5c34)n2)cc1]

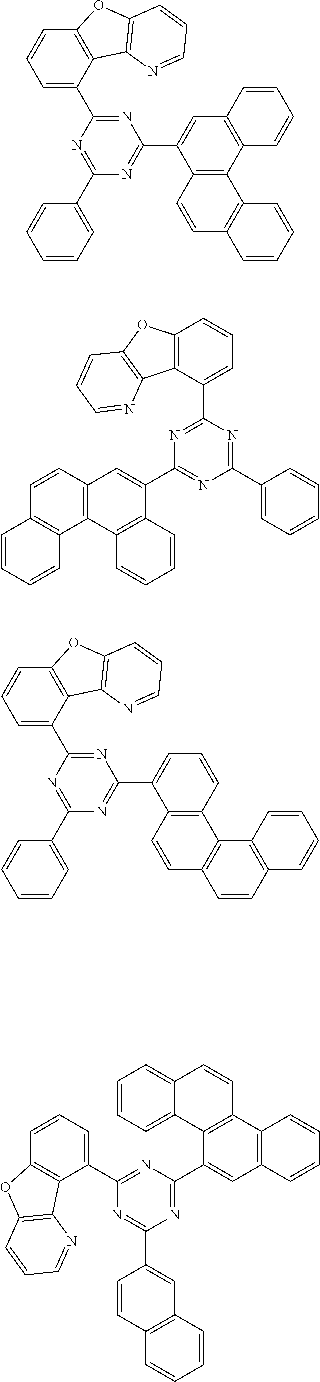 [SMILES: c1ccc(-c2nc(-c3cc4ccccc4c4c3ccc3ccccc34)nc(-c3cccc4oc5cccnc5c34)n2)cc1 ; c1ccc(-c2nc(-c3cc4ccc5ccccc5c4c4ccccc34)nc(-c3cccc4oc5cccnc5c34)n2)cc1 ; c1ccc(-c2nc(-c3cccc4c3ccc3ccc5ccccc5c34)nc(-c3cccc4oc5cccnc5c34)n2)cc1 ; c1ccc2cc(-c3nc(-c4cc5ccccc5c5ccc6ccccc6c45)nc(-c4cccc5oc6cccnc6c45)n3)ccc2c1]

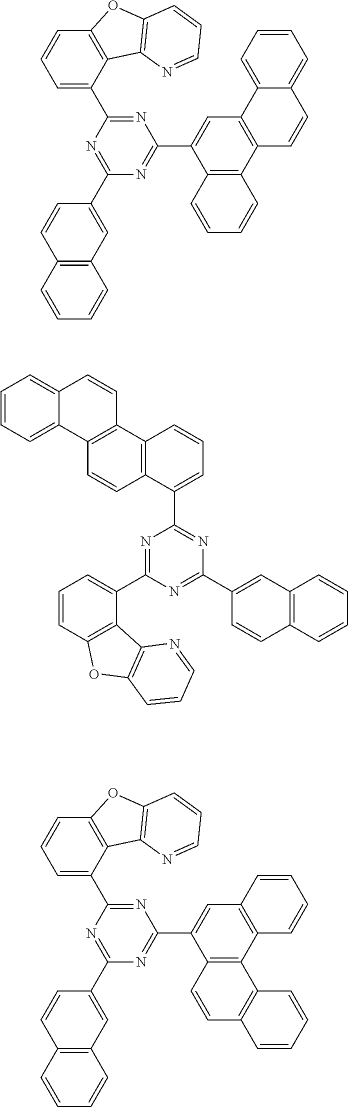 [SMILES: c1ccc2cc(-c3nc(-c4cc5c6ccccc6ccc5c5ccccc45)nc(-c4cccc5oc6cccnc6c45)n3)ccc2c1 ; c1ccc2cc(-c3nc(-c4cccc5c4ccc4c6ccccc6ccc54)nc(-c4cccc5oc6cccnc6c45)n3)ccc2c1 ; c1ccc2cc(-c3nc(-c4cc5ccccc5c5c4ccc4ccccc45)nc(-c4cccc5oc6cccnc6c45)n3)ccc2c1]

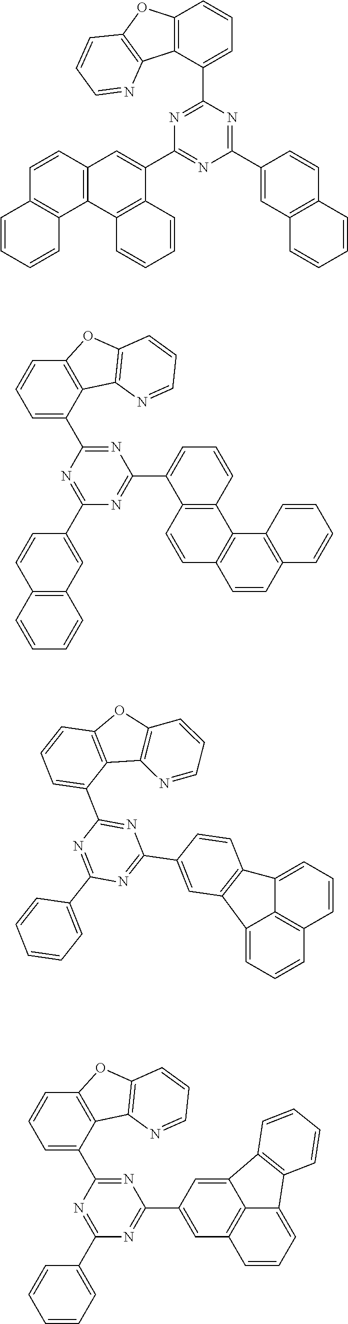 [SMILES: c1ccc2cc(-c3nc(-c4cc5ccc6ccccc6c5c5ccccc45)nc(-c4cccc5oc6cccnc6c45)n3)ccc2c1 ; c1ccc2cc(-c3nc(-c4cccc5c4ccc4ccc6ccccc6c45)nc(-c4cccc5oc6cccnc6c45)n3)ccc2c1 ; c1ccc(-c2nc(-c3ccc4c(c3)-c3cccc5cccc-4c35)nc(-c3cccc4oc5cccnc5c34)n2)cc1 ; c1ccc(-c2nc(-c3cc4c5c(cccc5c3)-c3ccccc3-4)nc(-c3cccc4oc5cccnc5c34)n2)cc1]

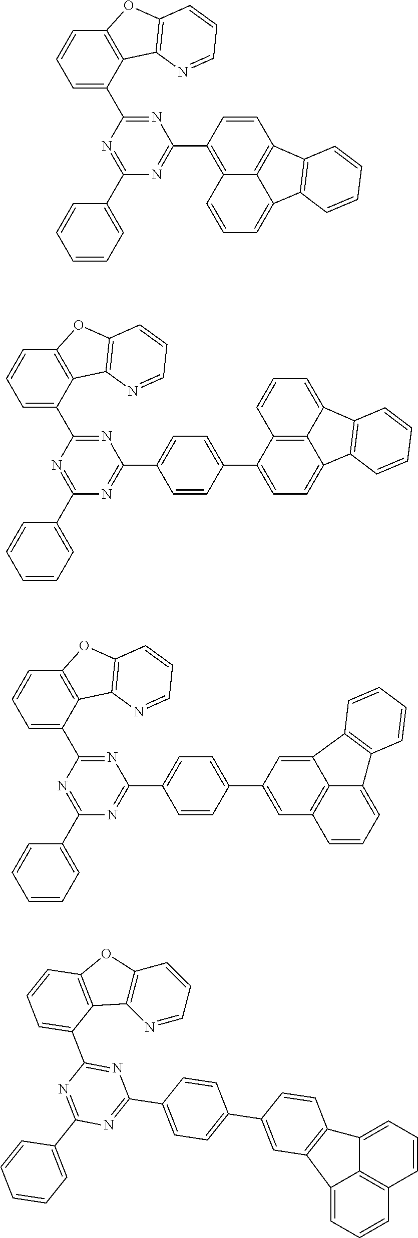 [SMILES: c1ccc(-c2nc(-c3ccc4c5c(cccc35)-c3ccccc3-4)nc(-c3cccc4oc5cccnc5c34)n2)cc1 ; c1ccc(-c2nc(-c3ccc(-c4ccc5c6c(cccc46)-c4ccccc4-5)cc3)nc(-c3cccc4oc5cccnc5c34)n2)cc1 ; c1ccc(-c2nc(-c3ccc(-c4cc5c6c(cccc6c4)-c4ccccc4-5)cc3)nc(-c3cccc4oc5cccnc5c34)n2)cc1 ; c1ccc(-c2nc(-c3ccc(-c4ccc5c(c4)-c4cccc6cccc-5c46)cc3)nc(-c3cccc4oc5cccnc5c34)n2)cc1]

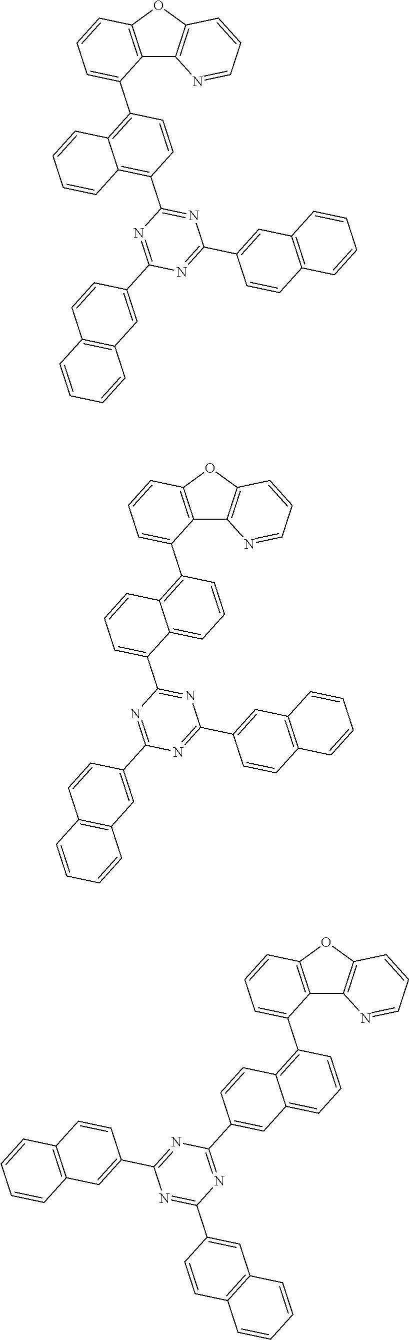 [SMILES: c1ccc2cc(-c3nc(-c4ccc5ccccc5c4)nc(-c4ccc(-c5cccc6oc7cccnc7c56)c5ccccc45)n3)ccc2c1 ; c1ccc2cc(-c3nc(-c4ccc5ccccc5c4)nc(-c4cccc5c(-c6cccc7oc8cccnc8c67)cccc45)n3)ccc2c1 ; c1ccc2cc(-c3nc(-c4ccc5ccccc5c4)nc(-c4ccc5c(-c6cccc7oc8cccnc8c67)cccc5c4)n3)ccc2c1]

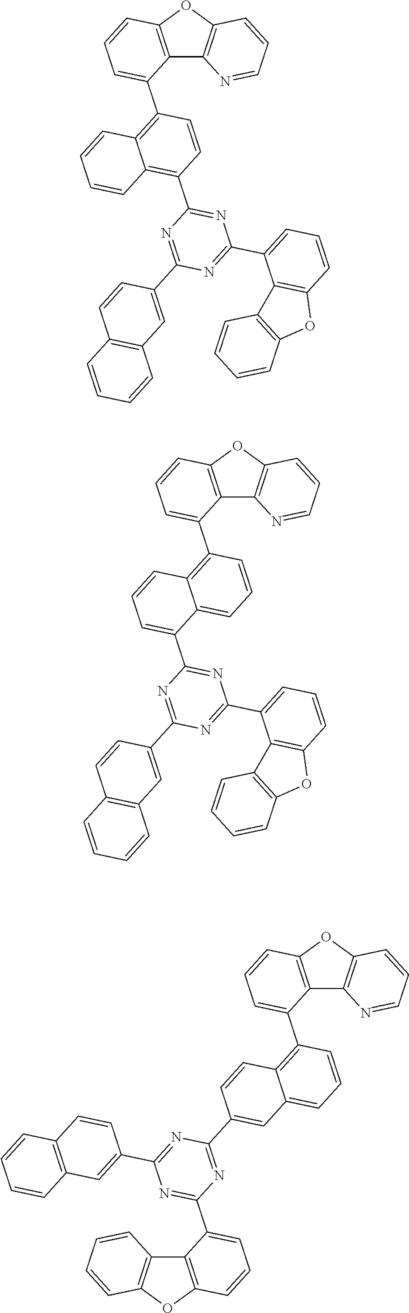 [SMILES: c1ccc2cc(-c3nc(-c4ccc(-c5cccc6oc7cccnc7c56)c5ccccc45)nc(-c4cccc5oc6ccccc6c45)n3)ccc2c1 ; c1ccc2cc(-c3nc(-c4cccc5c(-c6cccc7oc8cccnc8c67)cccc45)nc(-c4cccc5oc6ccccc6c45)n3)ccc2c1 ; c1ccc2cc(-c3nc(-c4ccc5c(-c6cccc7oc8cccnc8c67)cccc5c4)nc(-c4cccc5oc6ccccc6c45)n3)ccc2c1]

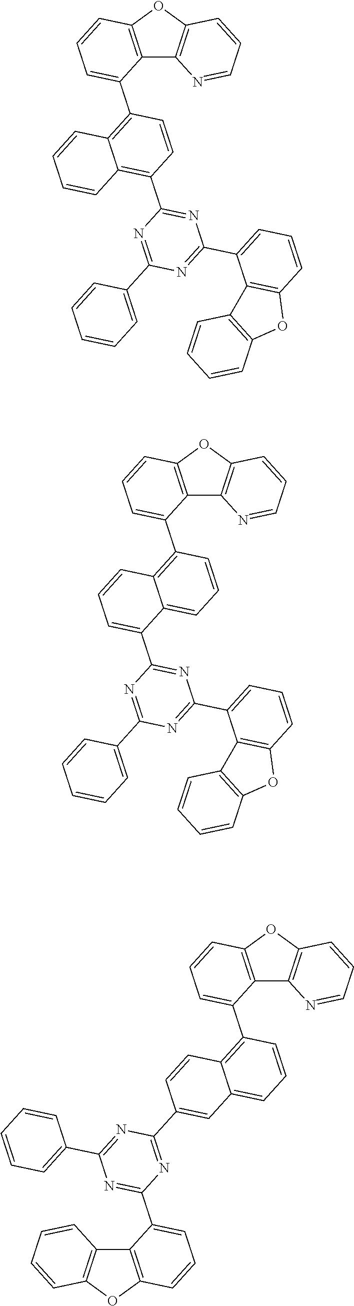 [SMILES: c1ccc(-c2nc(-c3ccc(-c4cccc5oc6cccnc6c45)c4ccccc34)nc(-c3cccc4oc5ccccc5c34)n2)cc1 ; c1ccc(-c2nc(-c3cccc4c(-c5cccc6oc7cccnc7c56)cccc34)nc(-c3cccc4oc5ccccc5c34)n2)cc1 ; c1ccc(-c2nc(-c3ccc4c(-c5cccc6oc7cccnc7c56)cccc4c3)nc(-c3cccc4oc5ccccc5c34)n2)cc1]

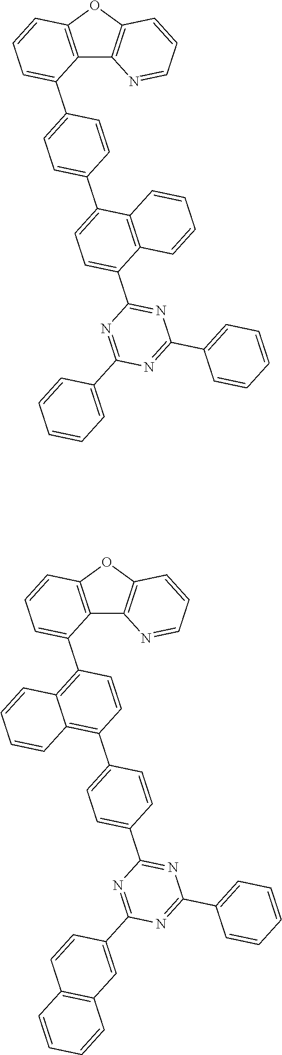 [SMILES: c1ccc(-c2nc(-c3ccccc3)nc(-c3ccc(-c4ccc(-c5cccc6oc7cccnc7c56)cc4)c4ccccc34)n2)cc1 ; c1ccc(-c2nc(-c3ccc(-c4ccc(-c5cccc6oc7cccnc7c56)c5ccccc45)cc3)nc(-c3ccc4ccccc4c3)n2)cc1]

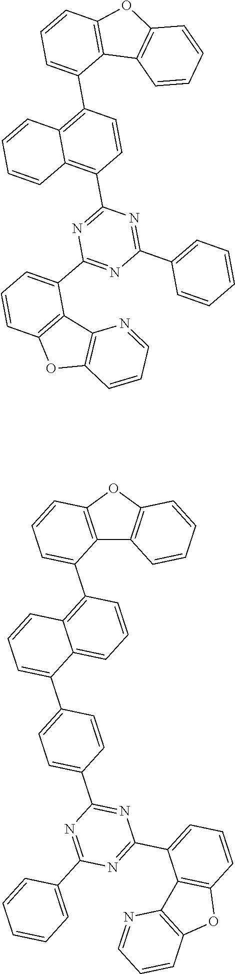 [SMILES: c1ccc(-c2nc(-c3ccc(-c4cccc5oc6ccccc6c45)c4ccccc34)nc(-c3cccc4oc5cccnc5c34)n2)cc1 ; c1ccc(-c2nc(-c3ccc(-c4cccc5c(-c6cccc7oc8ccccc8c67)cccc45)cc3)nc(-c3cccc4oc5cccnc5c34)n2)cc1]

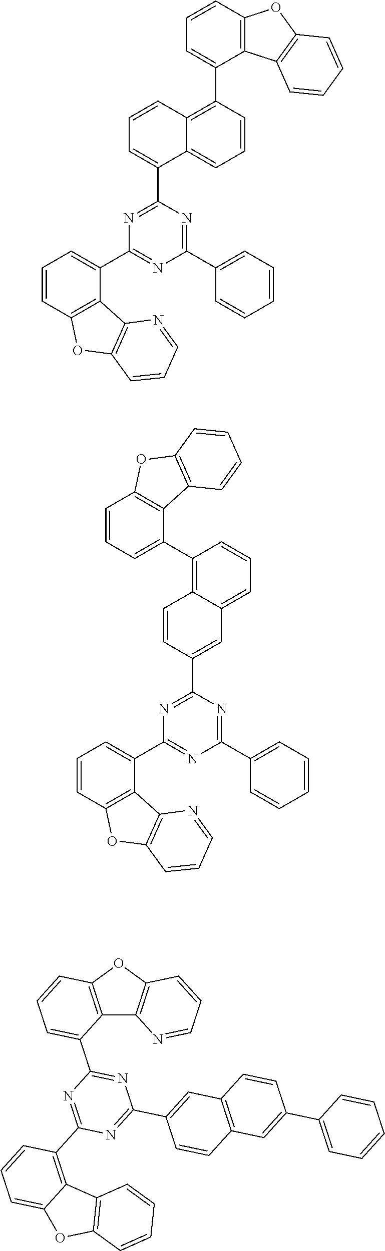 [SMILES: c1ccc(-c2nc(-c3cccc4c(-c5cccc6oc7ccccc7c56)cccc34)nc(-c3cccc4oc5cccnc5c34)n2)cc1 ; c1ccc(-c2nc(-c3ccc4c(-c5cccc6oc7ccccc7c56)cccc4c3)nc(-c3cccc4oc5cccnc5c34)n2)cc1 ; c1ccc(-c2ccc3cc(-c4nc(-c5cccc6oc7ccccc7c56)nc(-c5cccc6oc7cccnc7c56)n4)ccc3c2)cc1]

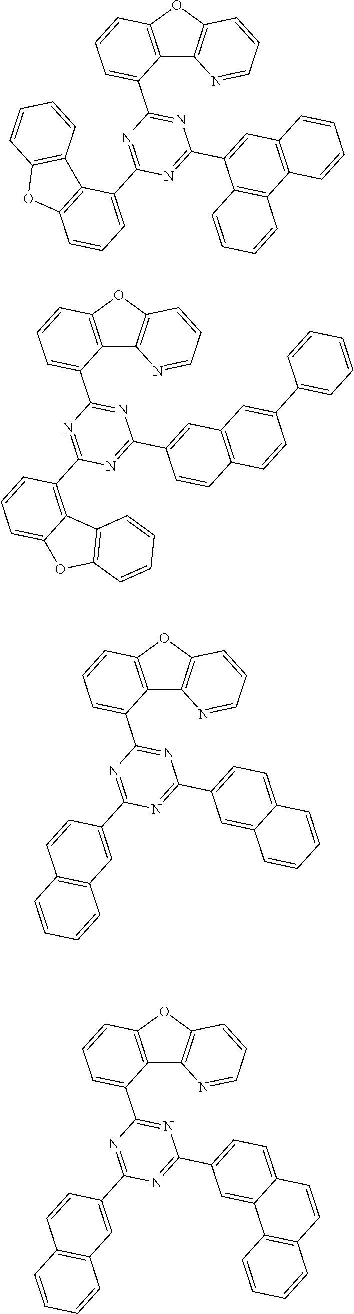 [SMILES: c1ccc2c(c1)cc(-c1nc(-c3cccc4oc5ccccc5c34)nc(-c3cccc4oc5cccnc5c34)n1)c1ccccc12 ; c1ccc(-c2ccc3ccc(-c4nc(-c5cccc6oc7ccccc7c56)nc(-c5cccc6oc7cccnc7c56)n4)cc3c2)cc1 ; c1ccc2cc(-c3nc(-c4ccc5ccccc5c4)nc(-c4cccc5oc6cccnc6c45)n3)ccc2c1 ; c1ccc2cc(-c3nc(-c4ccc5ccc6ccccc6c5c4)nc(-c4cccc5oc6cccnc6c45)n3)ccc2c1]

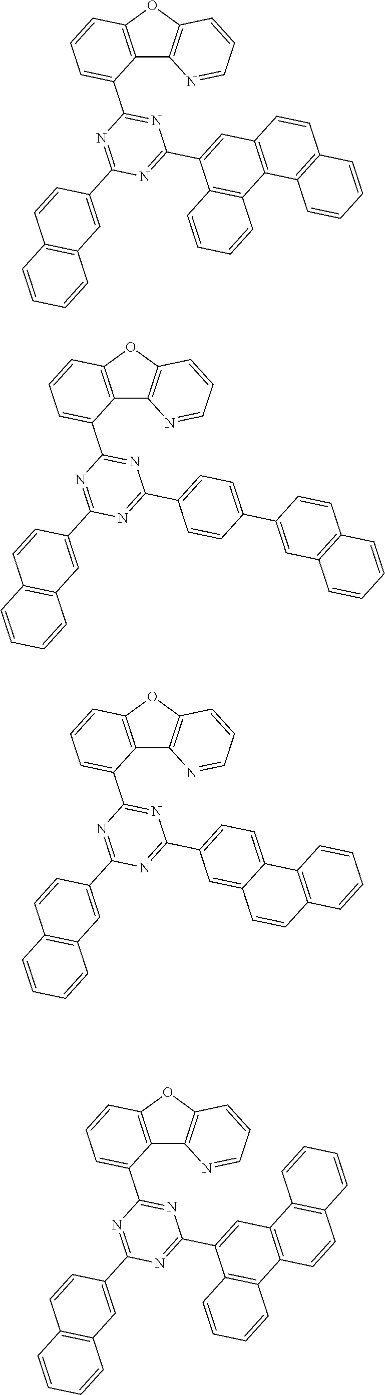 [SMILES: c1ccc2cc(-c3nc(-c4cc5ccc6ccccc6c5c5ccccc45)nc(-c4cccc5oc6cccnc6c45)n3)ccc2c1 ; c1ccc2cc(-c3ccc(-c4nc(-c5ccc6ccccc6c5)nc(-c5cccc6oc7cccnc7c56)n4)cc3)ccc2c1 ; c1ccc2cc(-c3nc(-c4ccc5c(ccc6ccccc65)c4)nc(-c4cccc5oc6cccnc6c45)n3)ccc2c1 ; c1ccc2cc(-c3nc(-c4cc5c6ccccc6ccc5c5ccccc45)nc(-c4cccc5oc6cccnc6c45)n3)ccc2c1]

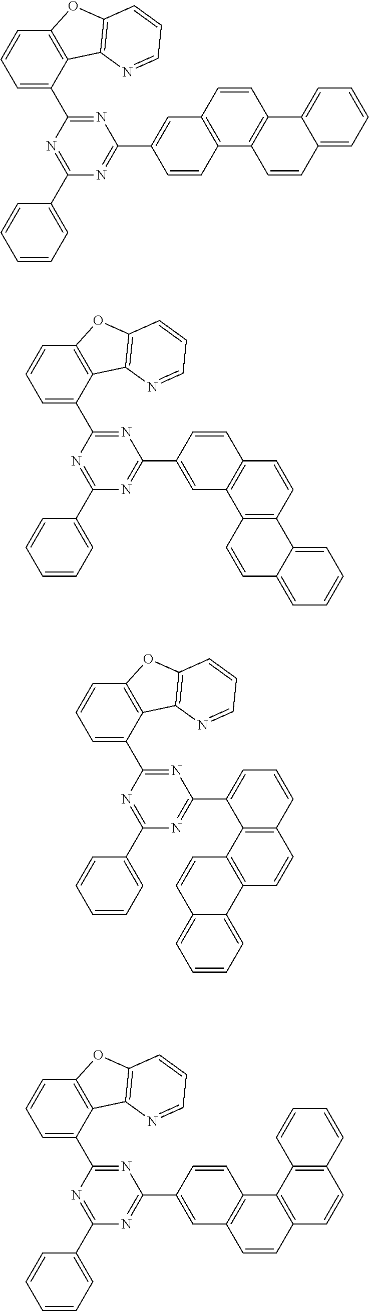 [SMILES: c1ccc(-c2nc(-c3ccc4c(ccc5c6ccccc6ccc45)c3)nc(-c3cccc4oc5cccnc5c34)n2)cc1 ; c1ccc(-c2nc(-c3ccc4ccc5c6ccccc6ccc5c4c3)nc(-c3cccc4oc5cccnc5c34)n2)cc1 ; c1ccc(-c2nc(-c3cccc4ccc5c6ccccc6ccc5c34)nc(-c3cccc4oc5cccnc5c34)n2)cc1 ; c1ccc(-c2nc(-c3ccc4c(ccc5ccc6ccccc6c54)c3)nc(-c3cccc4oc5cccnc5c34)n2)cc1]

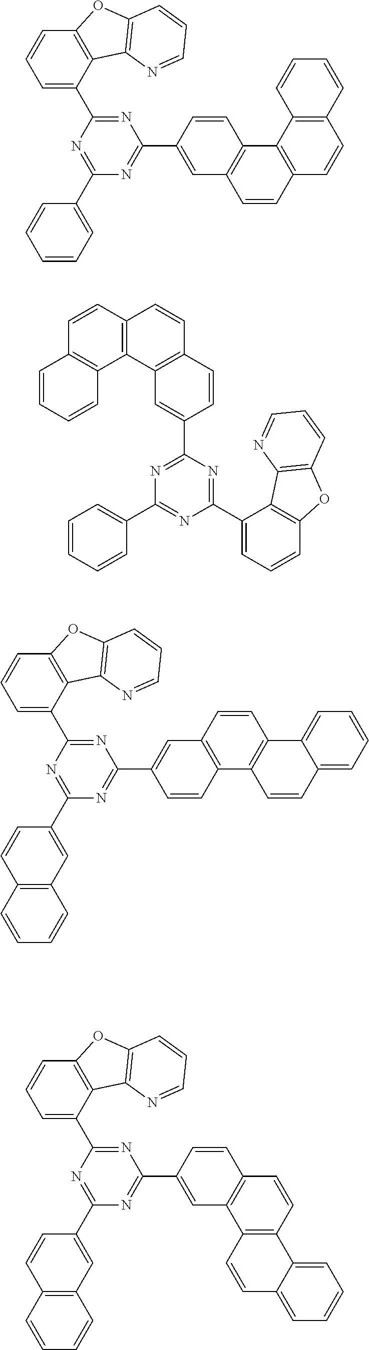 [SMILES: c1ccc(-c2nc(-c3ccc4c(ccc5ccc6ccccc6c54)c3)nc(-c3cccc4oc5cccnc5c34)n2)cc1 ; c1ccc(-c2nc(-c3ccc4ccc5ccc6ccccc6c5c4c3)nc(-c3cccc4oc5cccnc5c34)n2)cc1 ; c1ccc2cc(-c3nc(-c4ccc5c(ccc6c7ccccc7ccc56)c4)nc(-c4cccc5oc6cccnc6c45)n3)ccc2c1 ; c1ccc2cc(-c3nc(-c4ccc5ccc6c7ccccc7ccc6c5c4)nc(-c4cccc5oc6cccnc6c45)n3)ccc2c1]

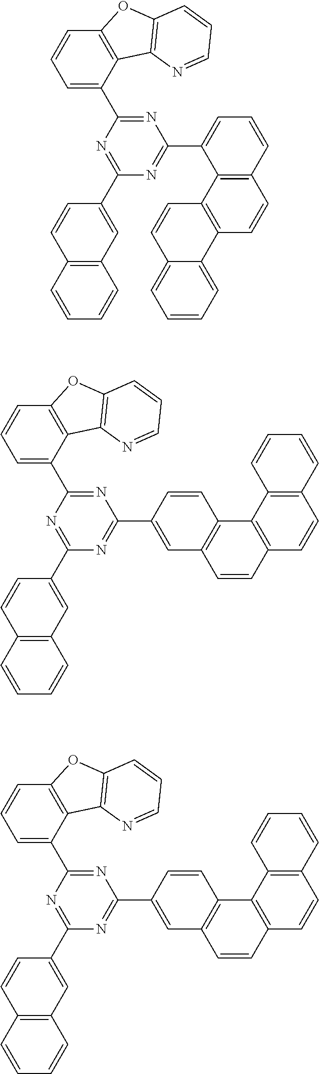 [SMILES: c1ccc2cc(-c3nc(-c4cccc5ccc6c7ccccc7ccc6c45)nc(-c4cccc5oc6cccnc6c45)n3)ccc2c1 ; c1ccc2cc(-c3nc(-c4ccc5c(ccc6ccc7ccccc7c65)c4)nc(-c4cccc5oc6cccnc6c45)n3)ccc2c1 ; c1ccc2cc(-c3nc(-c4ccc5c(ccc6ccc7ccccc7c65)c4)nc(-c4cccc5oc6cccnc6c45)n3)ccc2c1]

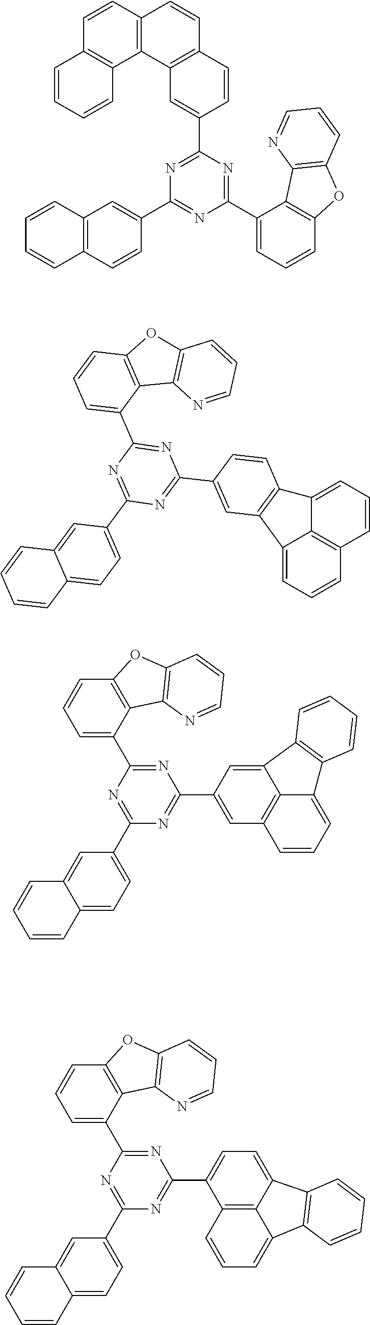 [SMILES: c1ccc2cc(-c3nc(-c4ccc5ccc6ccc7ccccc7c6c5c4)nc(-c4cccc5oc6cccnc6c45)n3)ccc2c1 ; c1ccc2cc(-c3nc(-c4ccc5c(c4)-c4cccc6cccc-5c46)nc(-c4cccc5oc6cccnc6c45)n3)ccc2c1 ; c1ccc2c(c1)-c1cccc3cc(-c4nc(-c5ccc6ccccc6c5)nc(-c5cccc6oc7cccnc7c56)n4)cc-2c13 ; c1ccc2c(c1)-c1cccc3c(-c4nc(-c5ccc6ccccc6c5)nc(-c5cccc6oc7cccnc7c56)n4)ccc-2c13]

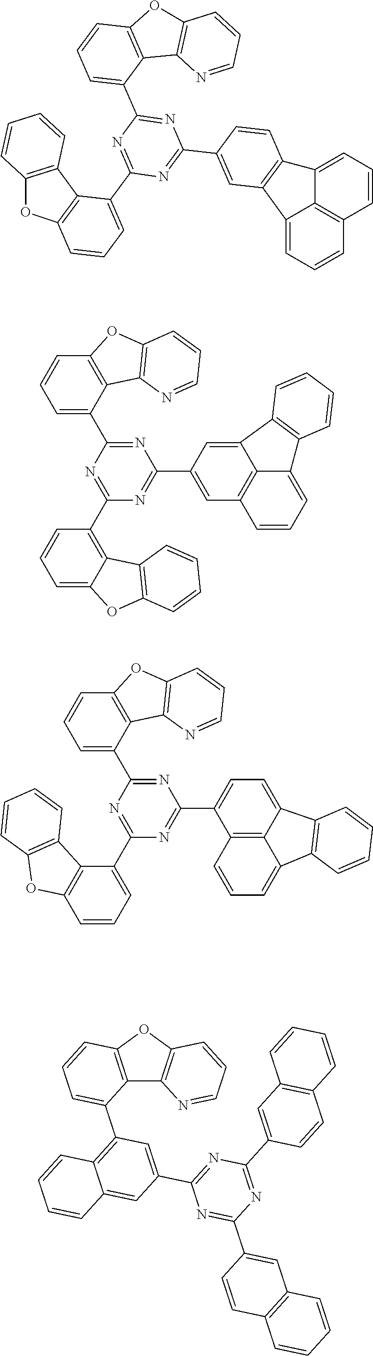 [SMILES: c1cc2c3c(cccc3c1)-c1cc(-c3nc(-c4cccc5oc6ccccc6c45)nc(-c4cccc5oc6cccnc6c45)n3)ccc1-2 ; c1ccc2c(c1)-c1cccc3cc(-c4nc(-c5cccc6oc7ccccc7c56)nc(-c5cccc6oc7cccnc7c56)n4)cc-2c13 ; c1ccc2c(c1)-c1cccc3c(-c4nc(-c5cccc6oc7ccccc7c56)nc(-c5cccc6oc7cccnc7c56)n4)ccc-2c13 ; c1ccc2cc(-c3nc(-c4ccc5ccccc5c4)nc(-c4cc(-c5cccc6oc7cccnc7c56)c5ccccc5c4)n3)ccc2c1]

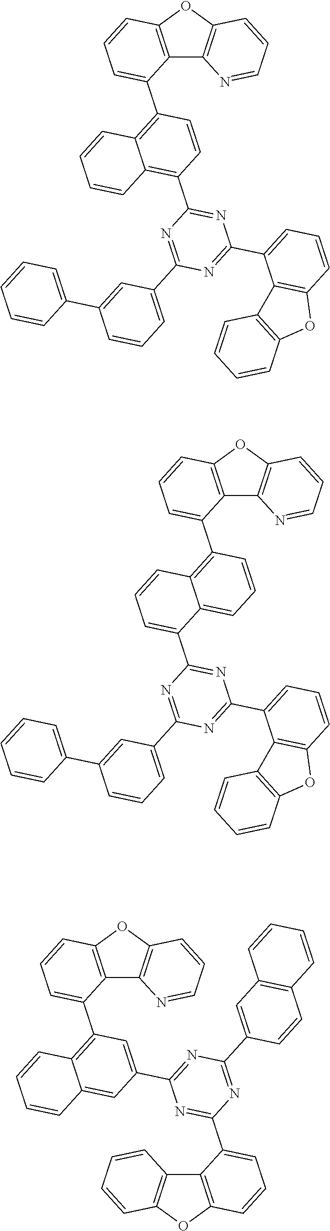 [SMILES: c1ccc(-c2cccc(-c3nc(-c4ccc(-c5cccc6oc7cccnc7c56)c5ccccc45)nc(-c4cccc5oc6ccccc6c45)n3)c2)cc1 ; c1ccc(-c2cccc(-c3nc(-c4cccc5c(-c6cccc7oc8cccnc8c67)cccc45)nc(-c4cccc5oc6ccccc6c45)n3)c2)cc1 ; c1ccc2cc(-c3nc(-c4cc(-c5cccc6oc7cccnc7c56)c5ccccc5c4)nc(-c4cccc5oc6ccccc6c45)n3)ccc2c1]

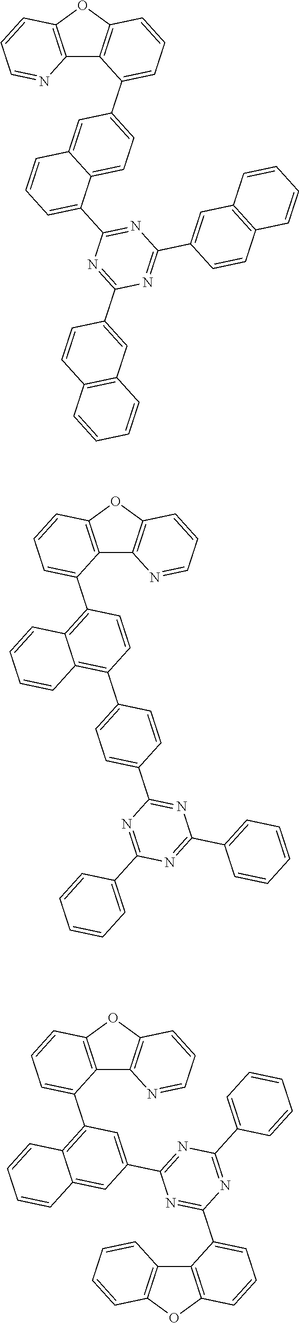 [SMILES: c1ccc2cc(-c3nc(-c4ccc5ccccc5c4)nc(-c4cccc5cc(-c6cccc7oc8cccnc8c67)ccc45)n3)ccc2c1 ; c1ccc(-c2nc(-c3ccccc3)nc(-c3ccc(-c4ccc(-c5cccc6oc7cccnc7c56)c5ccccc45)cc3)n2)cc1 ; c1ccc(-c2nc(-c3cc(-c4cccc5oc6cccnc6c45)c4ccccc4c3)nc(-c3cccc4oc5ccccc5c34)n2)cc1]

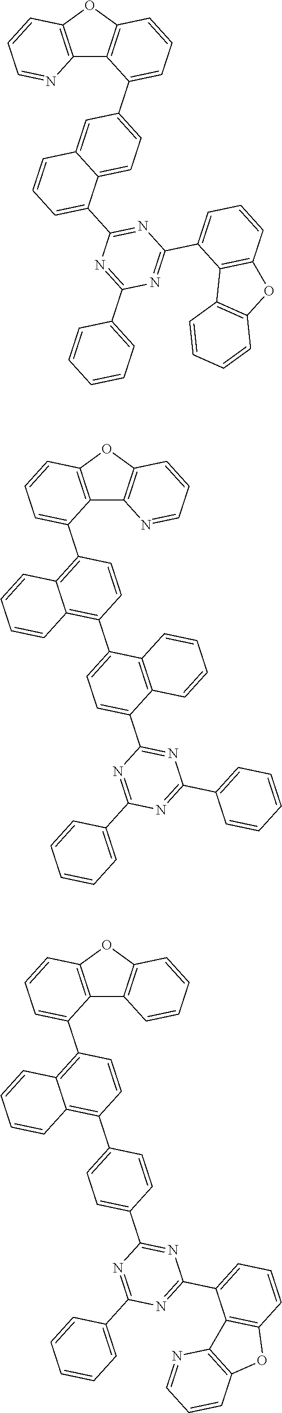 [SMILES: c1ccc(-c2nc(-c3cccc4cc(-c5cccc6oc7cccnc7c56)ccc34)nc(-c3cccc4oc5ccccc5c34)n2)cc1 ; c1ccc(-c2nc(-c3ccccc3)nc(-c3ccc(-c4ccc(-c5cccc6oc7cccnc7c56)c5ccccc45)c4ccccc34)n2)cc1 ; c1ccc(-c2nc(-c3ccc(-c4ccc(-c5cccc6oc7ccccc7c56)c5ccccc45)cc3)nc(-c3cccc4oc5cccnc5c34)n2)cc1]

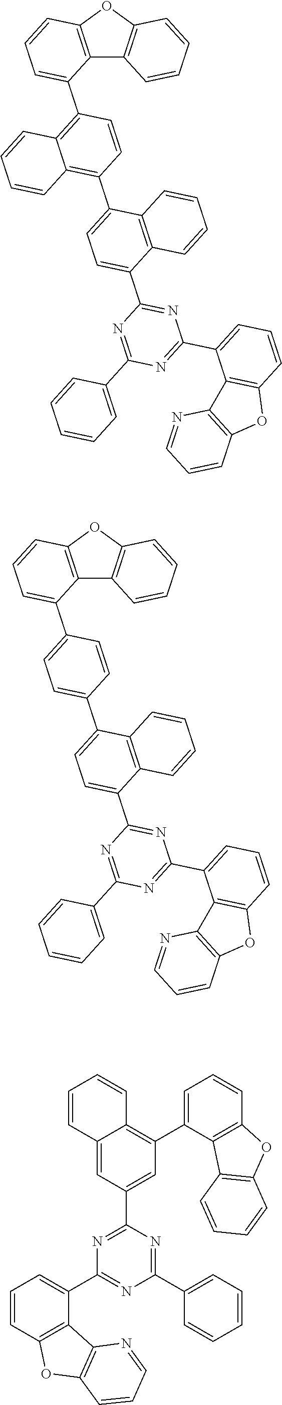 [SMILES: c1ccc(-c2nc(-c3ccc(-c4ccc(-c5cccc6oc7ccccc7c56)c5ccccc45)c4ccccc34)nc(-c3cccc4oc5cccnc5c34)n2)cc1 ; c1ccc(-c2nc(-c3ccc(-c4ccc(-c5cccc6oc7ccccc7c56)cc4)c4ccccc34)nc(-c3cccc4oc5cccnc5c34)n2)cc1 ; c1ccc(-c2nc(-c3cc(-c4cccc5oc6ccccc6c45)c4ccccc4c3)nc(-c3cccc4oc5cccnc5c34)n2)cc1]

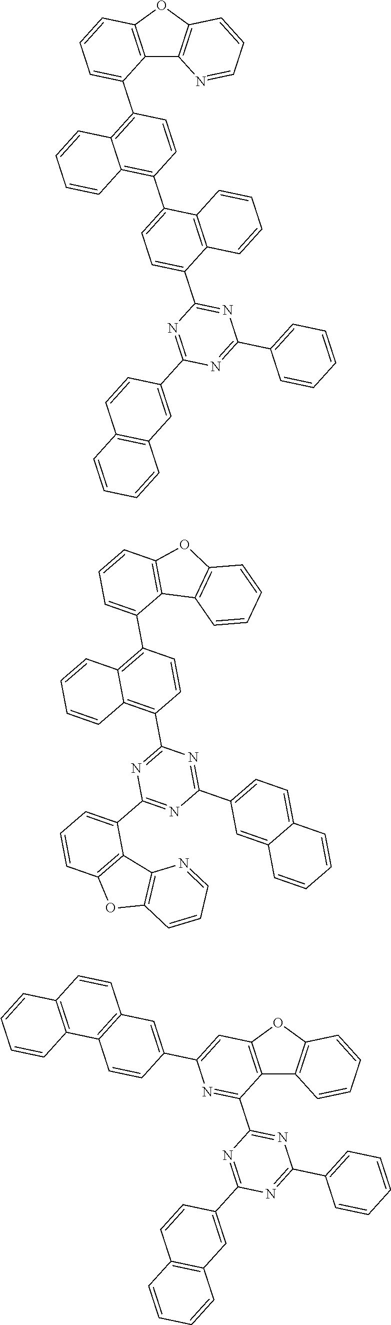 [SMILES: c1ccc(-c2nc(-c3ccc4ccccc4c3)nc(-c3ccc(-c4ccc(-c5cccc6oc7cccnc7c56)c5ccccc45)c4ccccc34)n2)cc1 ; c1ccc2cc(-c3nc(-c4ccc(-c5cccc6oc7ccccc7c56)c5ccccc45)nc(-c4cccc5oc6cccnc6c45)n3)ccc2c1 ; c1ccc(-c2nc(-c3ccc4ccccc4c3)nc(-c3nc(-c4ccc5c(ccc6ccccc65)c4)cc4oc5ccccc5c34)n2)cc1]

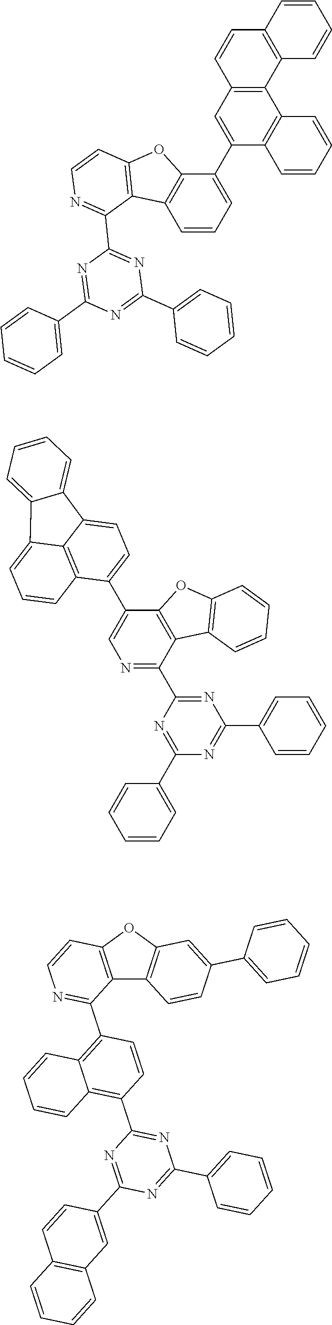 [SMILES: c1ccc(-c2nc(-c3ccccc3)nc(-c3nccc4oc5c(-c6cc7ccc8ccccc8c7c7ccccc67)cccc5c34)n2)cc1 ; c1ccc(-c2nc(-c3ccccc3)nc(-c3ncc(-c4ccc5c6c(cccc46)-c4ccccc4-5)c4oc5ccccc5c34)n2)cc1 ; c1ccc(-c2ccc3c(c2)oc2ccnc(-c4ccc(-c5nc(-c6ccccc6)nc(-c6ccc7ccccc7c6)n5)c5ccccc45)c23)cc1]

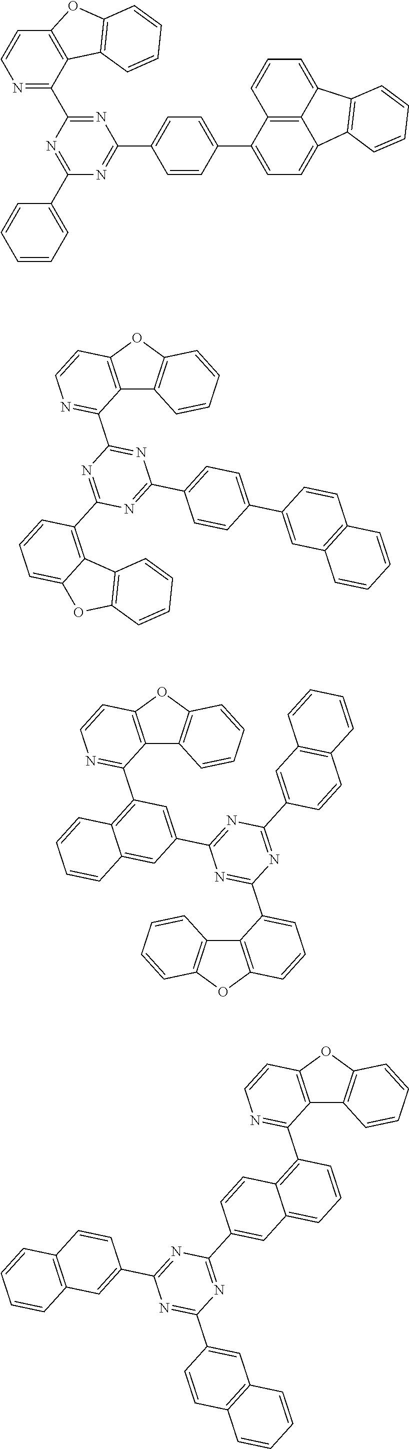 [SMILES: c1ccc(-c2nc(-c3ccc(-c4ccc5c6c(cccc46)-c4ccccc4-5)cc3)nc(-c3nccc4oc5ccccc5c34)n2)cc1 ; c1ccc2cc(-c3ccc(-c4nc(-c5cccc6oc7ccccc7c56)nc(-c5nccc6oc7ccccc7c56)n4)cc3)ccc2c1 ; c1ccc2cc(-c3nc(-c4cc(-c5nccc6oc7ccccc7c56)c5ccccc5c4)nc(-c4cccc5oc6ccccc6c45)n3)ccc2c1 ; c1ccc2cc(-c3nc(-c4ccc5ccccc5c4)nc(-c4ccc5c(-c6nccc7oc8ccccc8c67)cccc5c4)n3)ccc2c1]

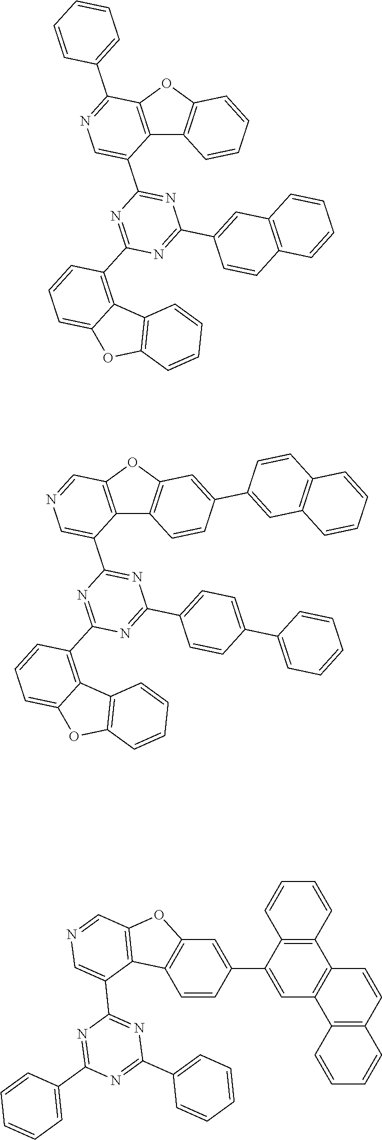 [SMILES: c1ccc(-c2ncc(-c3nc(-c4ccc5ccccc5c4)nc(-c4cccc5oc6ccccc6c45)n3)c3c2oc2ccccc23)cc1 ; c1ccc(-c2ccc(-c3nc(-c4cccc5oc6ccccc6c45)nc(-c4cncc5oc6cc(-c7ccc8ccccc8c7)ccc6c45)n3)cc2)cc1 ; c1ccc(-c2nc(-c3ccccc3)nc(-c3cncc4oc5cc(-c6cc7c8ccccc8ccc7c7ccccc67)ccc5c34)n2)cc1]

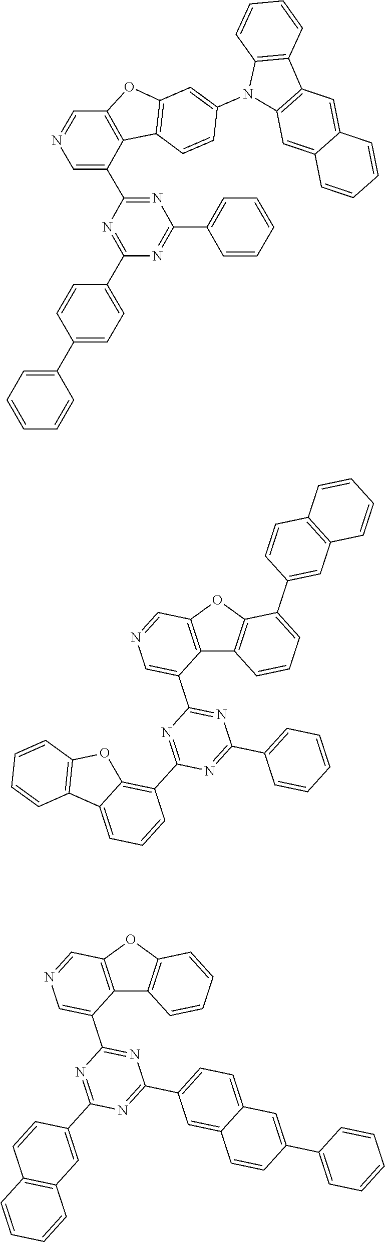 [SMILES: c1ccc(-c2ccc(-c3nc(-c4ccccc4)nc(-c4cncc5oc6cc(-n7c8ccccc8c8cc9ccccc9cc87)ccc6c45)n3)cc2)cc1 ; c1ccc(-c2nc(-c3cccc4c3oc3ccccc34)nc(-c3cncc4oc5c(-c6ccc7ccccc7c6)cccc5c34)n2)cc1 ; c1ccc(-c2ccc3cc(-c4nc(-c5ccc6ccccc6c5)nc(-c5cncc6oc7ccccc7c56)n4)ccc3c2)cc1]

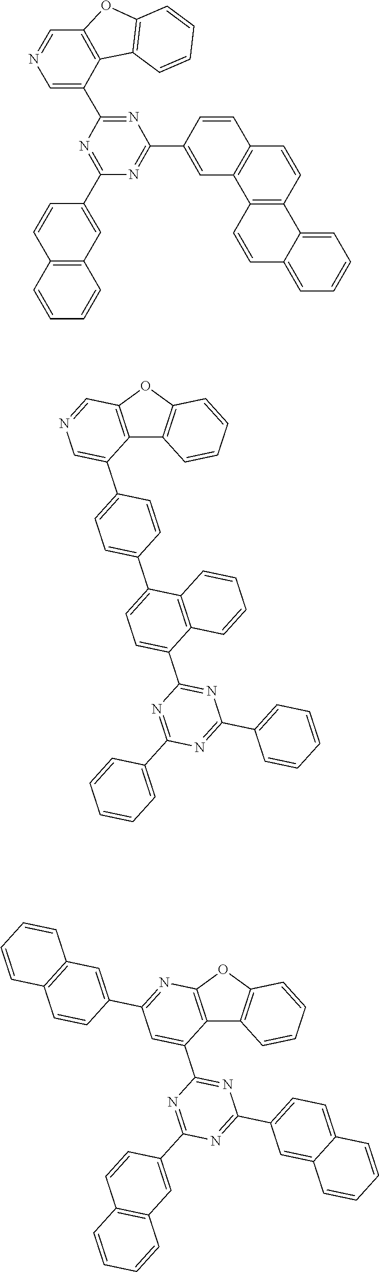 [SMILES: c1ccc2cc(-c3nc(-c4ccc5ccc6c7ccccc7ccc6c5c4)nc(-c4cncc5oc6ccccc6c45)n3)ccc2c1 ; c1ccc(-c2nc(-c3ccccc3)nc(-c3ccc(-c4ccc(-c5cncc6oc7ccccc7c56)cc4)c4ccccc34)n2)cc1 ; c1ccc2cc(-c3cc(-c4nc(-c5ccc6ccccc6c5)nc(-c5ccc6ccccc6c5)n4)c4c(n3)oc3ccccc34)ccc2c1]

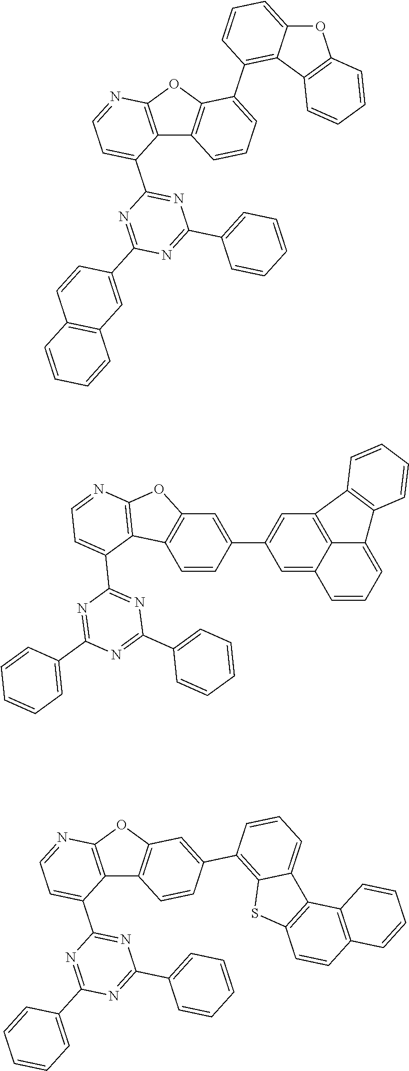 [SMILES: c1ccc(-c2nc(-c3ccc4ccccc4c3)nc(-c3ccnc4oc5c(-c6cccc7oc8ccccc8c67)cccc5c34)n2)cc1 ; c1ccc(-c2nc(-c3ccccc3)nc(-c3ccnc4oc5cc(-c6cc7c8c(cccc8c6)-c6ccccc6-7)ccc5c34)n2)cc1 ; c1ccc(-c2nc(-c3ccccc3)nc(-c3ccnc4oc5cc(-c6cccc7c6sc6ccc8ccccc8c67)ccc5c34)n2)cc1]

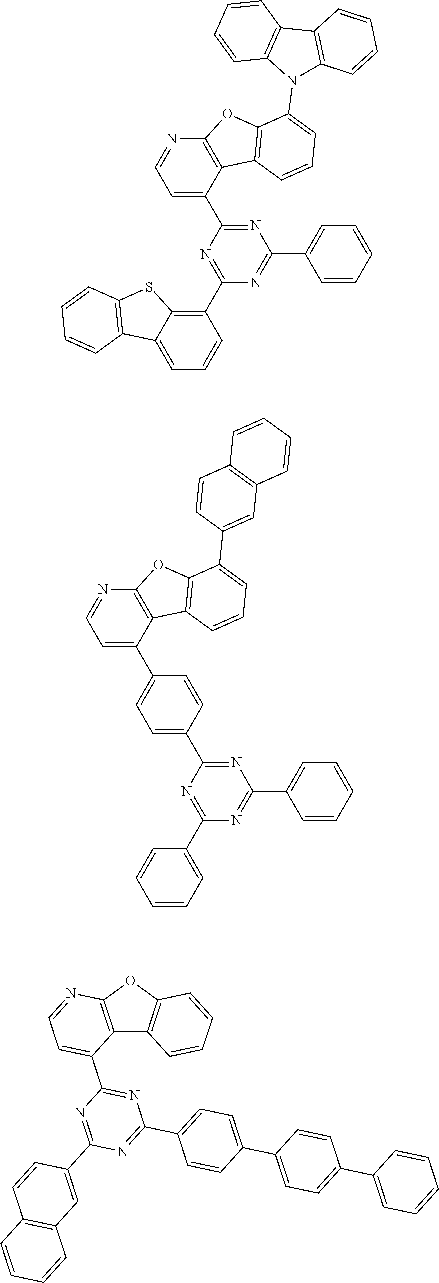 [SMILES: c1ccc(-c2nc(-c3cccc4c3sc3ccccc34)nc(-c3ccnc4oc5c(-n6c7ccccc7c7ccccc76)cccc5c34)n2)cc1 ; c1ccc(-c2nc(-c3ccccc3)nc(-c3ccc(-c4ccnc5oc6c(-c7ccc8ccccc8c7)cccc6c45)cc3)n2)cc1 ; c1ccc(-c2ccc(-c3ccc(-c4nc(-c5ccc6ccccc6c5)nc(-c5ccnc6oc7ccccc7c56)n4)cc3)cc2)cc1]

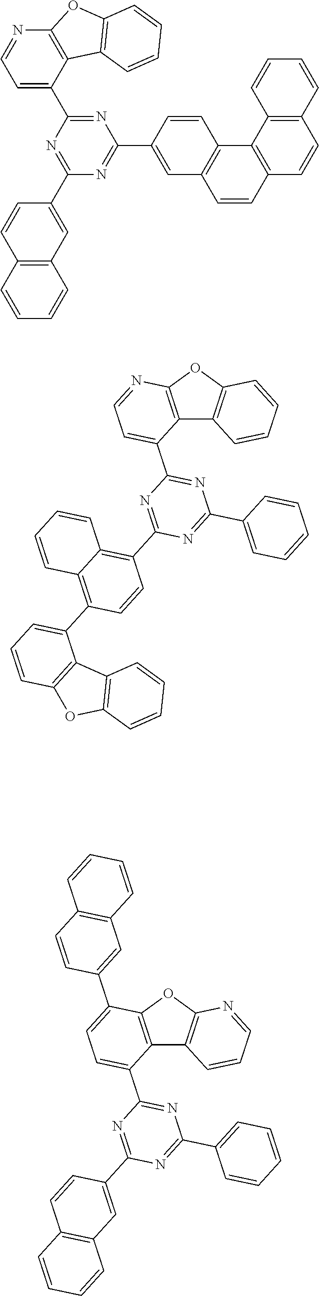 [SMILES: c1ccc2cc(-c3nc(-c4ccc5c(ccc6ccc7ccccc7c65)c4)nc(-c4ccnc5oc6ccccc6c45)n3)ccc2c1 ; c1ccc(-c2nc(-c3ccc(-c4cccc5oc6ccccc6c45)c4ccccc34)nc(-c3ccnc4oc5ccccc5c34)n2)cc1 ; c1ccc(-c2nc(-c3ccc4ccccc4c3)nc(-c3ccc(-c4ccc5ccccc5c4)c4oc5ncccc5c34)n2)cc1]

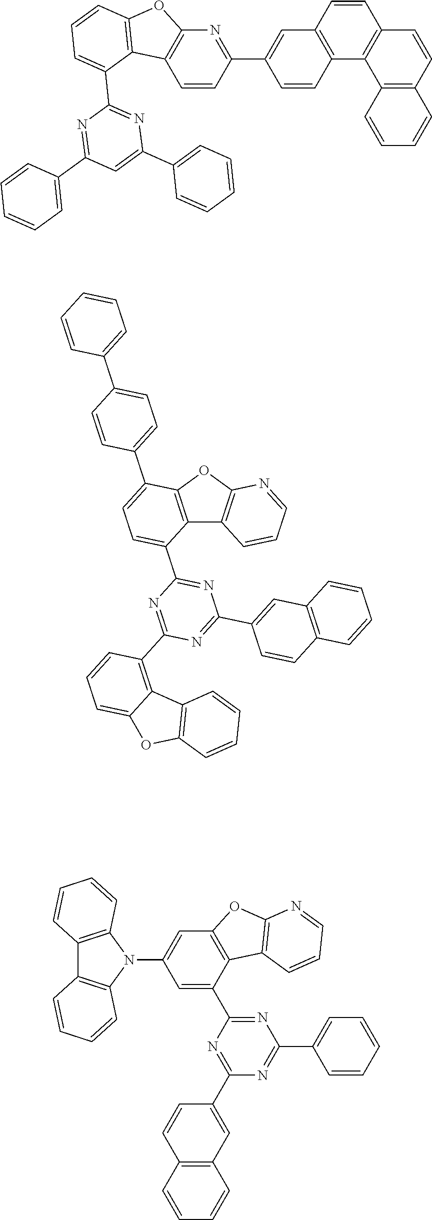 [SMILES: c1ccc(-c2cc(-c3ccccc3)nc(-c3cccc4oc5nc(-c6ccc7c(ccc8ccc9ccccc9c87)c6)ccc5c34)n2)cc1 ; c1ccc(-c2ccc(-c3ccc(-c4nc(-c5ccc6ccccc6c5)nc(-c5cccc6oc7ccccc7c56)n4)c4c3oc3ncccc34)cc2)cc1 ; c1ccc(-c2nc(-c3ccc4ccccc4c3)nc(-c3cc(-n4c5ccccc5c5ccccc54)cc4oc5ncccc5c34)n2)cc1]

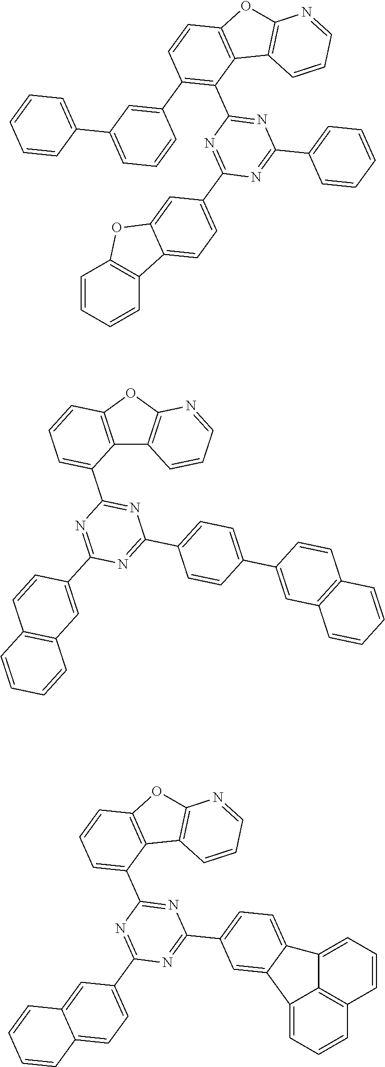 [SMILES: c1ccc(-c2cccc(-c3ccc4oc5ncccc5c4c3-c3nc(-c4ccccc4)nc(-c4ccc5c(c4)oc4ccccc45)n3)c2)cc1 ; c1ccc2cc(-c3ccc(-c4nc(-c5ccc6ccccc6c5)nc(-c5cccc6oc7ncccc7c56)n4)cc3)ccc2c1 ; c1ccc2cc(-c3nc(-c4ccc5c(c4)-c4cccc6cccc-5c46)nc(-c4cccc5oc6ncccc6c45)n3)ccc2c1]

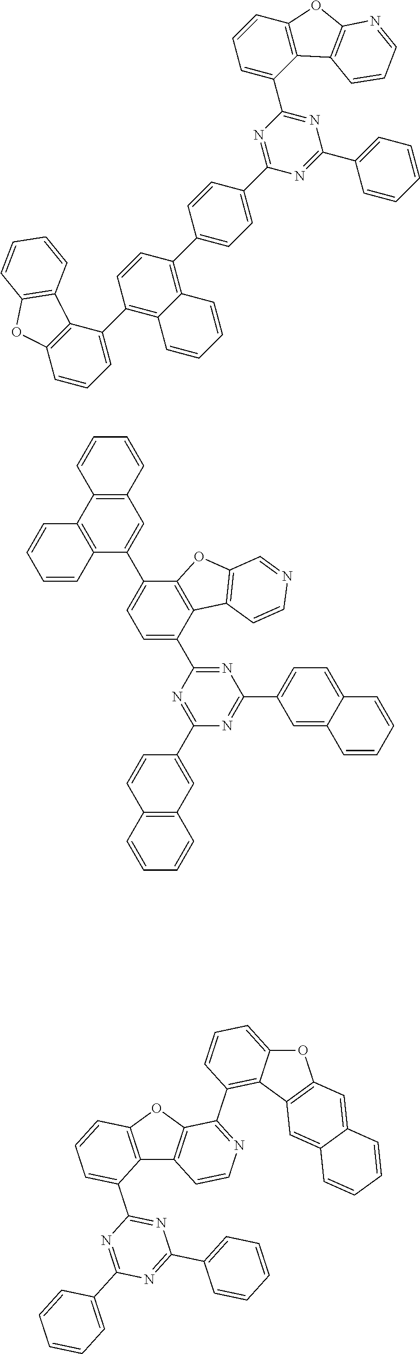 [SMILES: c1ccc(-c2nc(-c3ccc(-c4ccc(-c5cccc6oc7ccccc7c56)c5ccccc45)cc3)nc(-c3cccc4oc5ncccc5c34)n2)cc1 ; c1ccc2cc(-c3nc(-c4ccc5ccccc5c4)nc(-c4ccc(-c5cc6ccccc6c6ccccc56)c5oc6cnccc6c45)n3)ccc2c1 ; c1ccc(-c2nc(-c3ccccc3)nc(-c3cccc4oc5c(-c6cccc7oc8cc9ccccc9cc8c67)nccc5c34)n2)cc1]

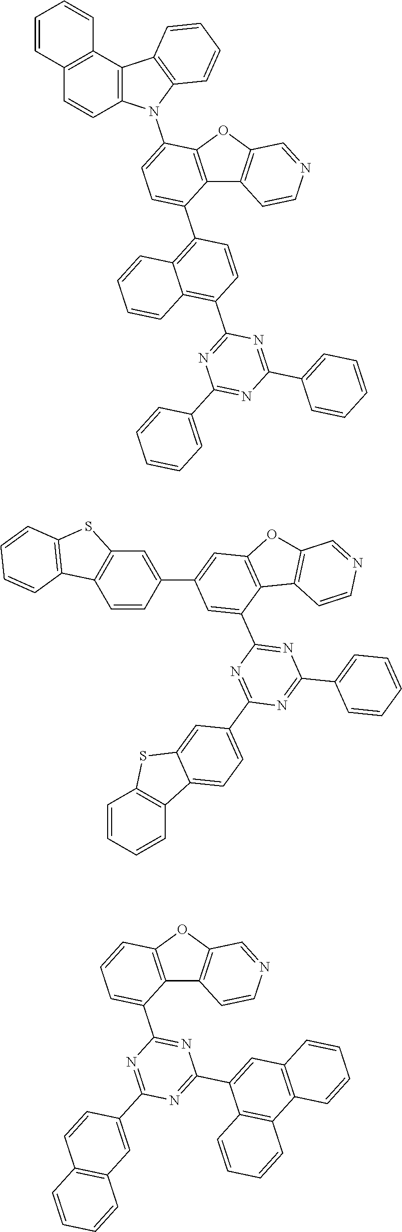 [SMILES: c1ccc(-c2nc(-c3ccccc3)nc(-c3ccc(-c4ccc(-n5c6ccccc6c6c7ccccc7ccc65)c5oc6cnccc6c45)c4ccccc34)n2)cc1 ; c1ccc(-c2nc(-c3ccc4c(c3)sc3ccccc34)nc(-c3cc(-c4ccc5c(c4)sc4ccccc45)cc4oc5cnccc5c34)n2)cc1 ; c1ccc2cc(-c3nc(-c4cc5ccccc5c5ccccc45)nc(-c4cccc5oc6cnccc6c45)n3)ccc2c1]

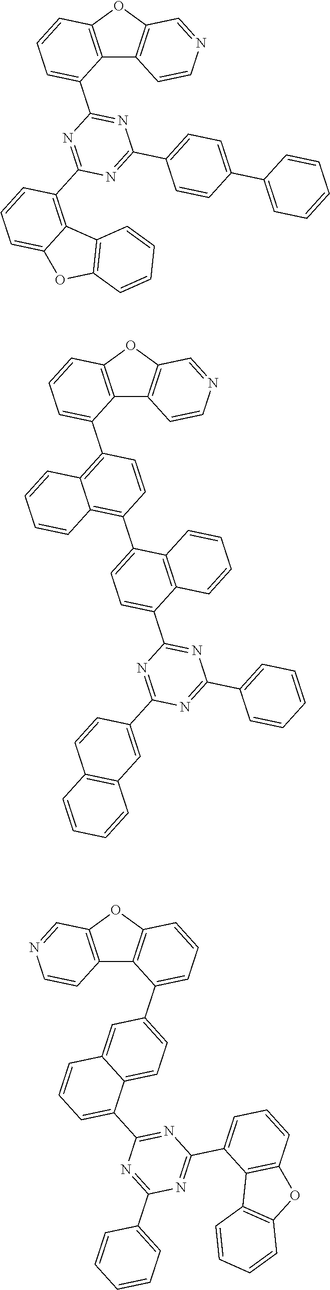 [SMILES: c1ccc(-c2ccc(-c3nc(-c4cccc5oc6ccccc6c45)nc(-c4cccc5oc6cnccc6c45)n3)cc2)cc1 ; c1ccc(-c2nc(-c3ccc4ccccc4c3)nc(-c3ccc(-c4ccc(-c5cccc6oc7cnccc7c56)c5ccccc45)c4ccccc34)n2)cc1 ; c1ccc(-c2nc(-c3cccc4cc(-c5cccc6oc7cnccc7c56)ccc34)nc(-c3cccc4oc5ccccc5c34)n2)cc1]

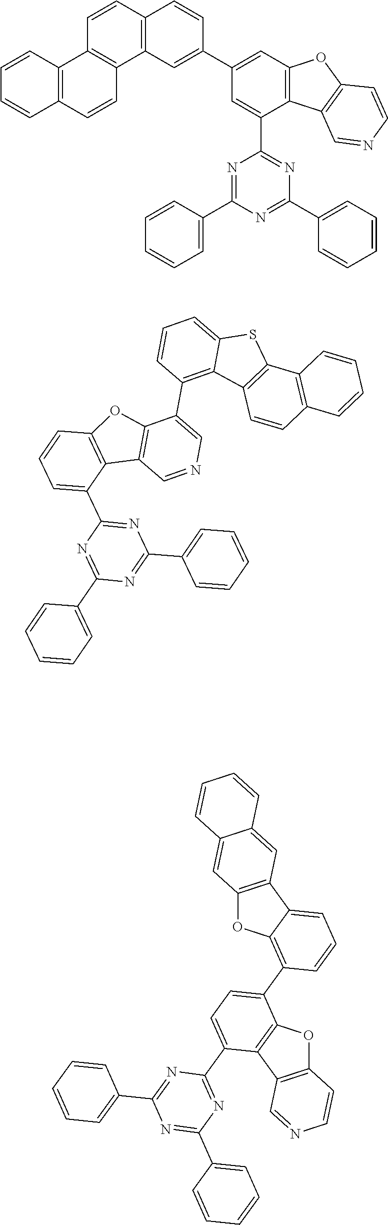 [SMILES: c1ccc(-c2nc(-c3ccccc3)nc(-c3cc(-c4ccc5ccc6c7ccccc7ccc6c5c4)cc4oc5ccncc5c34)n2)cc1 ; c1ccc(-c2nc(-c3ccccc3)nc(-c3cccc4oc5c(-c6cccc7sc8c9ccccc9ccc8c67)cncc5c34)n2)cc1 ; c1ccc(-c2nc(-c3ccccc3)nc(-c3ccc(-c4cccc5c4oc4cc6ccccc6cc45)c4oc5ccncc5c34)n2)cc1]

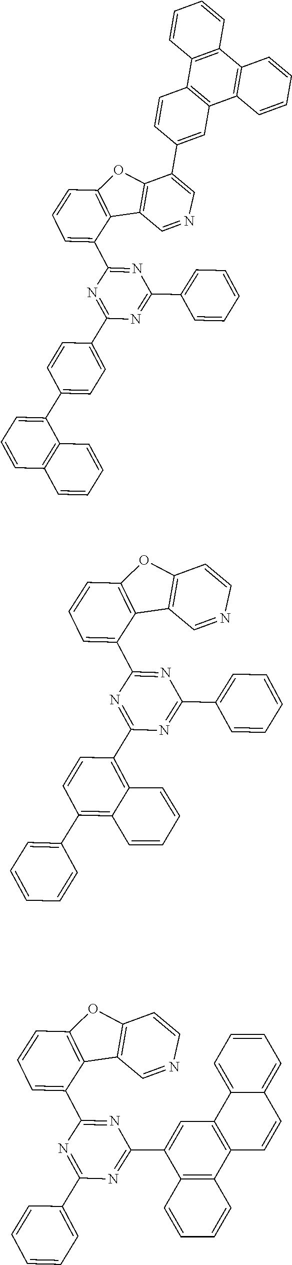 [SMILES: c1ccc(-c2nc(-c3ccc(-c4cccc5ccccc45)cc3)nc(-c3cccc4oc5c(-c6ccc7c8ccccc8c8ccccc8c7c6)cncc5c34)n2)cc1 ; c1ccc(-c2nc(-c3ccc(-c4ccccc4)c4ccccc34)nc(-c3cccc4oc5ccncc5c34)n2)cc1 ; c1ccc(-c2nc(-c3cc4c5ccccc5ccc4c4ccccc34)nc(-c3cccc4oc5ccncc5c34)n2)cc1]

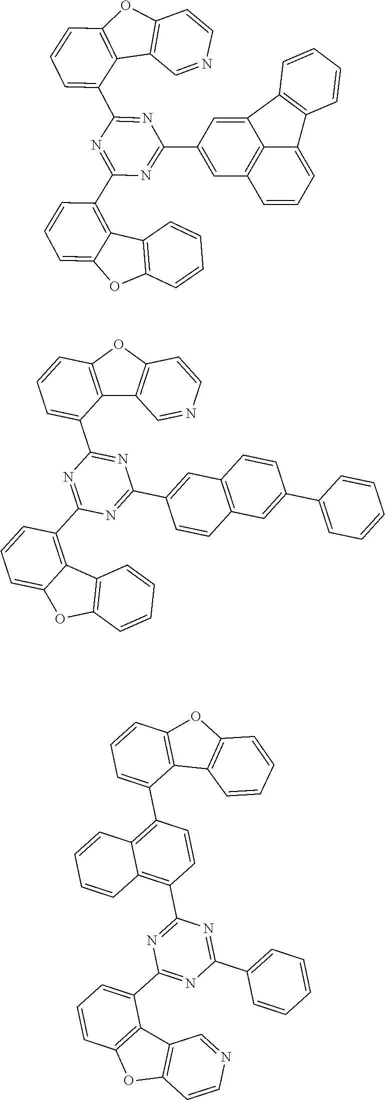 [SMILES: c1ccc2c(c1)-c1cccc3cc(-c4nc(-c5cccc6oc7ccccc7c56)nc(-c5cccc6oc7ccncc7c56)n4)cc-2c13 ; c1ccc(-c2ccc3cc(-c4nc(-c5cccc6oc7ccccc7c56)nc(-c5cccc6oc7ccncc7c56)n4)ccc3c2)cc1 ; c1ccc(-c2nc(-c3ccc(-c4cccc5oc6ccccc6c45)c4ccccc34)nc(-c3cccc4oc5ccncc5c34)n2)cc1]

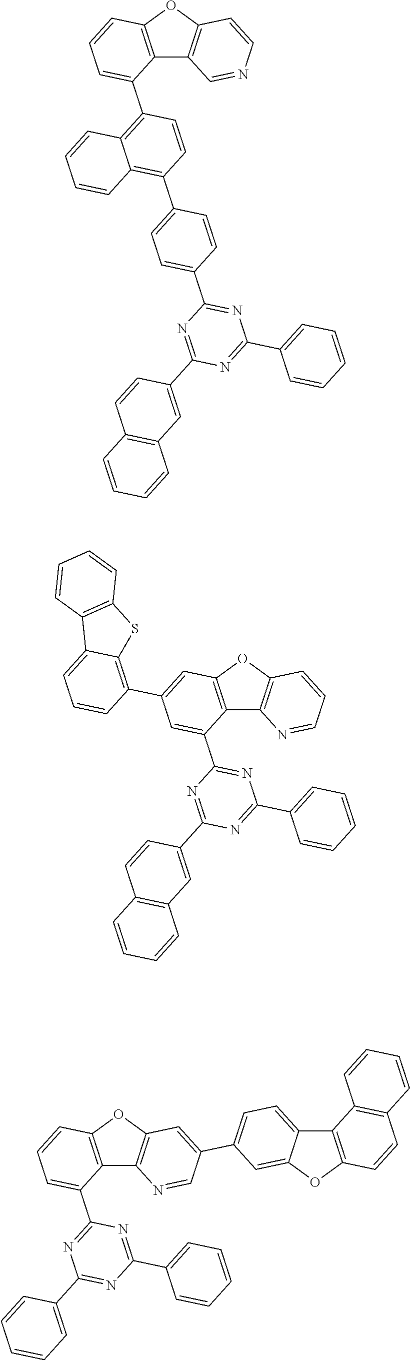 [SMILES: c1ccc(-c2nc(-c3ccc(-c4ccc(-c5cccc6oc7ccncc7c56)c5ccccc45)cc3)nc(-c3ccc4ccccc4c3)n2)cc1 ; c1ccc(-c2nc(-c3ccc4ccccc4c3)nc(-c3cc(-c4cccc5c4sc4ccccc45)cc4oc5cccnc5c34)n2)cc1 ; c1ccc(-c2nc(-c3ccccc3)nc(-c3cccc4oc5cc(-c6ccc7c(c6)oc6ccc8ccccc8c67)cnc5c34)n2)cc1]

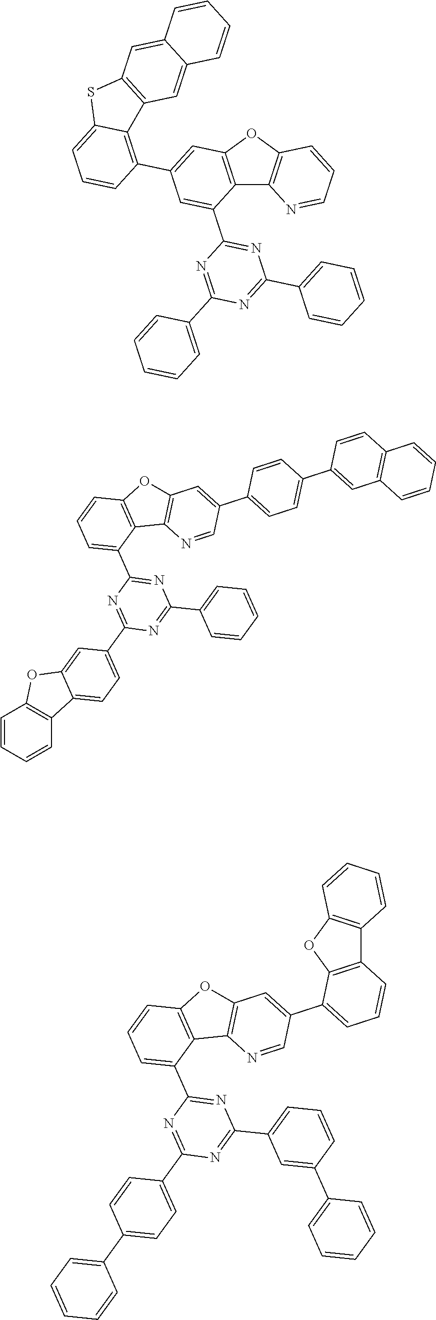 [SMILES: c1ccc(-c2nc(-c3ccccc3)nc(-c3cc(-c4cccc5sc6cc7ccccc7cc6c45)cc4oc5cccnc5c34)n2)cc1 ; c1ccc(-c2nc(-c3ccc4c(c3)oc3ccccc34)nc(-c3cccc4oc5cc(-c6ccc(-c7ccc8ccccc8c7)cc6)cnc5c34)n2)cc1 ; c1ccc(-c2ccc(-c3nc(-c4cccc(-c5ccccc5)c4)nc(-c4cccc5oc6cc(-c7cccc8c7oc7ccccc78)cnc6c45)n3)cc2)cc1]

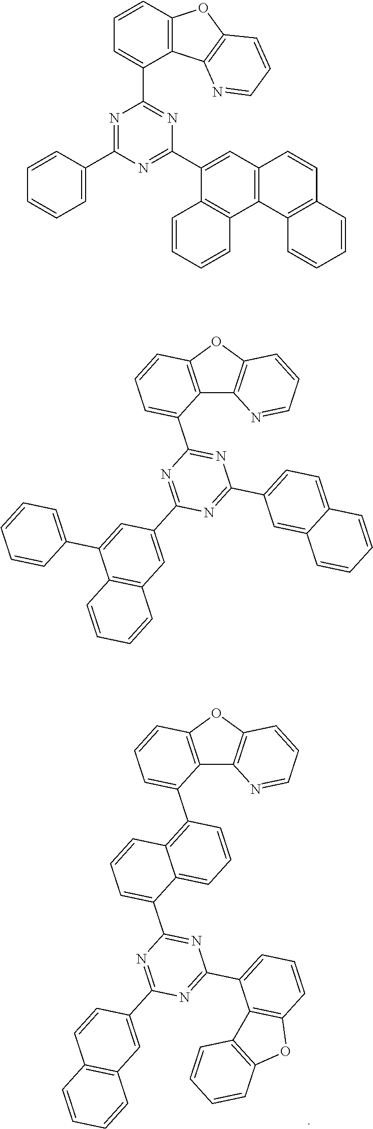 [SMILES: c1ccc(-c2nc(-c3cc4ccc5ccccc5c4c4ccccc34)nc(-c3cccc4oc5cccnc5c34)n2)cc1 ; c1ccc(-c2cc(-c3nc(-c4ccc5ccccc5c4)nc(-c4cccc5oc6cccnc6c45)n3)cc3ccccc23)cc1 ; c1ccc2cc(-c3nc(-c4cccc5c(-c6cccc7oc8cccnc8c67)cccc45)nc(-c4cccc5oc6ccccc6c45)n3)ccc2c1]

The compound of Chemical Formula 1 wherein X₁ is N, X₂ is CR₁, and X₃ to X₇ are CH can be prepared by a preparation method as shown in the following Reaction Scheme 1-1 as an example, the compound wherein X₁ is N, and X₂ to X₇ is CH can be prepared by a preparation method as shown in the following Reaction Scheme 1-2 as an example, and the other remaining compounds can be prepared in a similar manner.

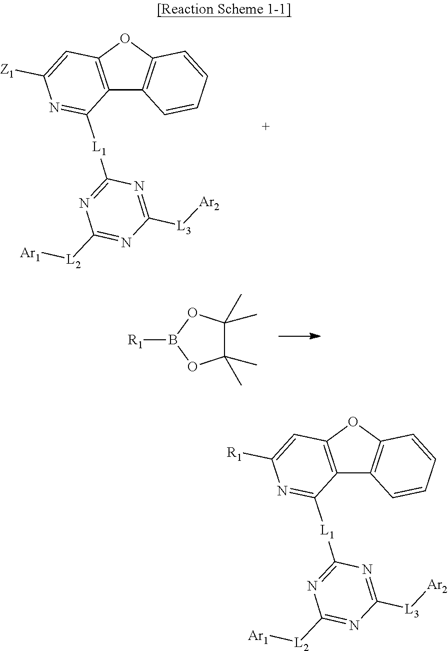

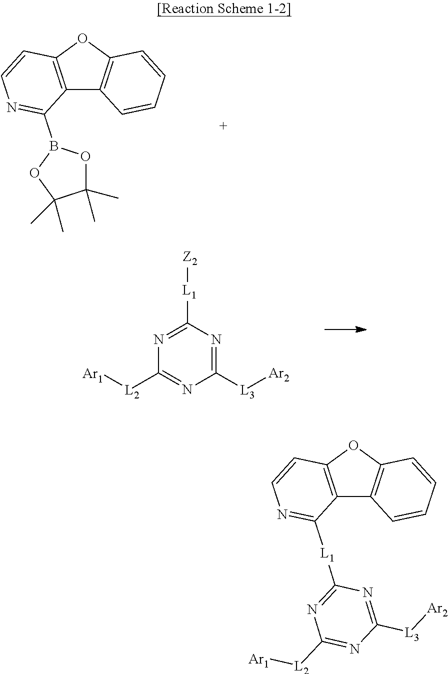

In Reaction Schemes 1-1 and 1-2, R₁, L₁ to L₃, Ar₁ and Ar₂ are the same as defined in Chemical Formula 1, Z₁ and Z₂ are each independently halogen, preferably Z₁ and Z₂ are each independently chloro or bromo.

Reaction Schemes 1-1 and 1-2 are Suzuki coupling reactions, which are preferably carried out in the presence of a palladium catalyst and a base, and a reactive group for the Suzuki coupling reaction can be modified as known in the art. In addition, if necessary, an amine substitution reaction can be accompanied, and in this case, it is preferably carried out in the presence of a palladium catalyst and a base, and a reactive group for the amine substitution reaction can be modified as known in the art. The preparation method can be further embodied in the Preparation Examples described hereinafter.

Preferably, L₄ and L₅ are each independently a single bond, a substituted or unsubstituted C₆₋₂₀ arylene, or a substituted or unsubstituted C₂₋₂₀ heteroarylene containing at least one heteroatom selected from the group consisting of N, O and S. Preferably, L₄ and L₅ can be each independently a single bond, phenylene that is unsubstituted or substituted with 1 to 4 deuteriums, or biphenyldiyl that is unsubstituted or substituted with 1 to 8 deuteriums. More preferably, L₄ and L₅ can be each independently a single bond, phenylene, biphenyldiyl, or phenylene substituted with 4 deuteriums.

Preferably, A and B are each independently hydrogen, deuterium,

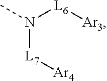

a substituted or unsubstituted C₆₋₂₀ aryl, or a substituted or unsubstituted C₂₋₂₀ heteroaryl containing any one or more heteroatoms selected from the group consisting of N, O and S, provided that at least one of A and B are

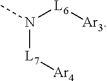

More preferably, A and B are each independently hydrogen, phenyl that is unsubstituted or substituted with 1 to 5 deuteriums, or

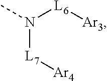

provided that at least one of A and B is

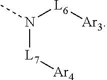

More preferably, A is

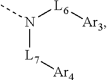

B can be hydrogen or phenyl that is unsubstituted or substituted with 1 to 5 deuteriums. Most preferably, A is

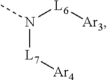

and B can be hydrogen or phenyl.

Preferably, L₆ and L₇ can be each independently a single bond, a substituted or unsubstituted C₆₋₂₀ arylene, or a substituted or unsubstituted C₂₋₂₀ heteroarylene containing at least one heteroatom selected from the group consisting of N, O and S. Preferably, L₆ and L₇ can be each independently a single bond, phenylene that is unsubstituted or substituted with 1 to 4 deuteriums, or biphenyldiyl that is unsubstituted or substituted with 1 to 8 deuteriums. More preferably, L₆ and L₇ can be each independently a single bond, phenylene, biphenyldiyl, or phenylene substituted with 4 deuteriums.

Preferably, Ar₃ and Ar₄ can be each independently a substituted or unsubstituted C₆₋₂₀ aryl or a substituted or unsubstituted C₂₋₂₀ heteroaryl containing at least one heteroatom selected from the group consisting of N, O and S. Preferably, Ar₃ and Ar₄ can be each independently phenyl, biphenylyl, terphenylyl, naphthyl, phenyl naphthyl, phenanthrenyl, triphenylenyl, phenyl phenanthrenyl, dimethyl fluorenyl, diphenylfluorenyl, dibenzofuranyl, dibenzothiophenyl, methyl dibenzofluorenyl, carbazolyl, or phenyl carbazolyl, wherein the Ar₃ and Ar₄ can be each independently unsubstituted or substituted with at least one deuterium. More preferably, Ar₃ and Ar₄ can be each independently phenyl, biphenylyl, terphenylyl, naphthyl, phenyl naphthyl, phenanthrenyl, triphenylenyl, phenyl phenanthrenyl, dimethyl fluorenyl, diphenylfluorenyl, dibenzofuranyl, dibenzothiophenyl, methyl dibenzofluorenyl, carbazolyl, phenyl carbazolyl, phenyl substituted with 5 deuteriums, biphenylyl substituted with 4 deuteriums, biphenylyl substituted with 9 deuteriums, or terphenylyl substituted with 4 deuteriums.

Preferably, each R₂ can be hydrogen.

Representative examples of the compound represented by Chemical Formula 2 are as follows:

[chemical structure]

[chemical structure]

[chemical structure]

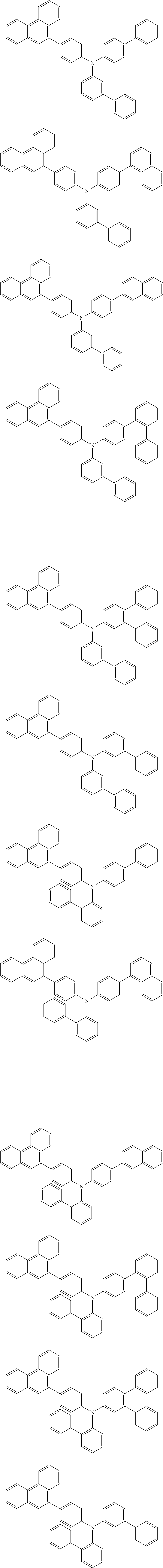

[chemical structure]

[chemical structure]

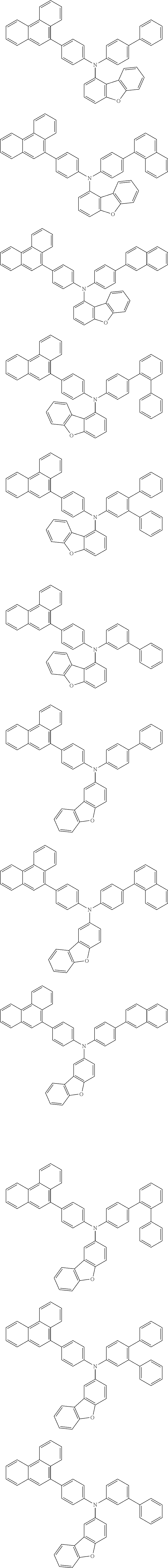

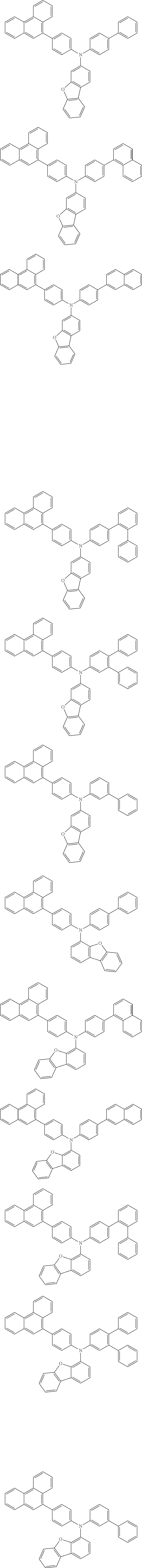

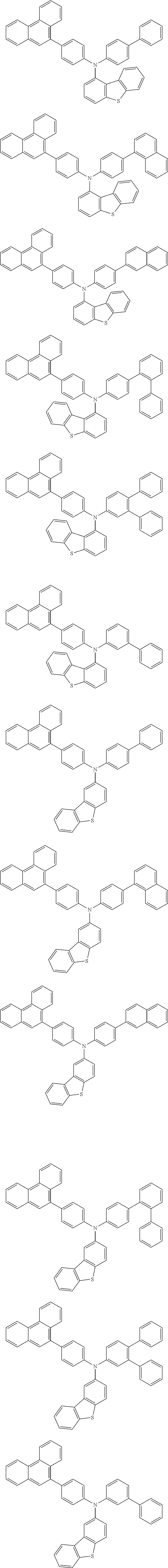

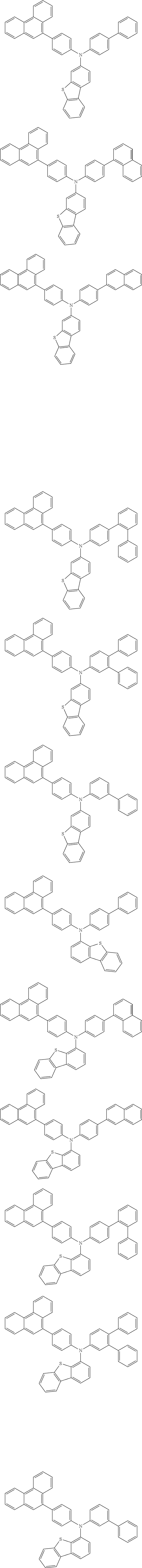

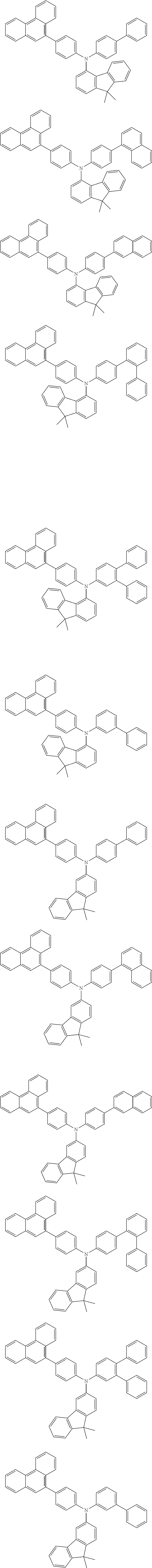

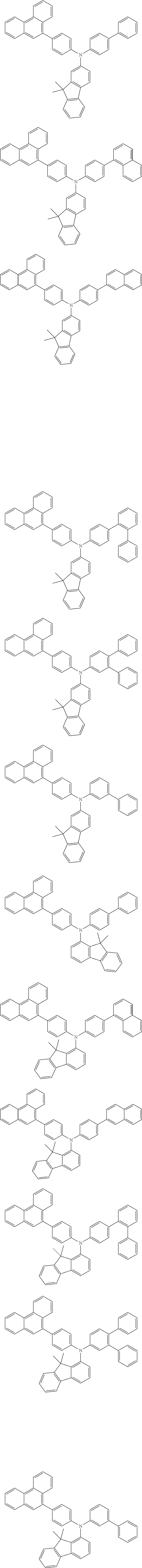

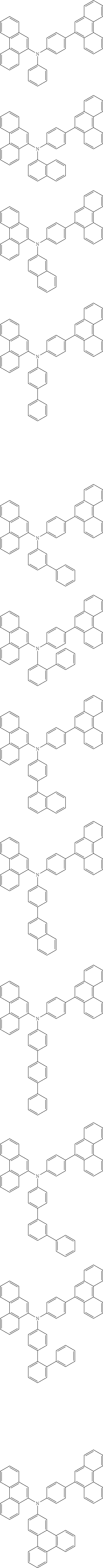

[chemical structure]

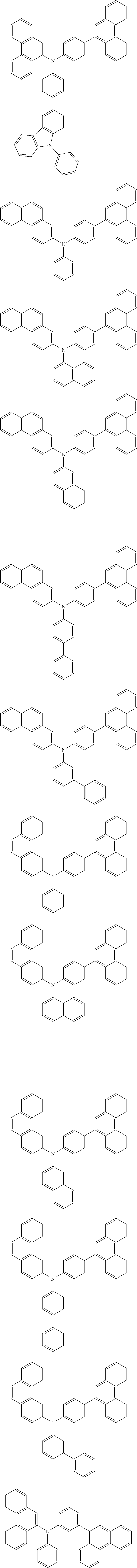

[chemical structure]

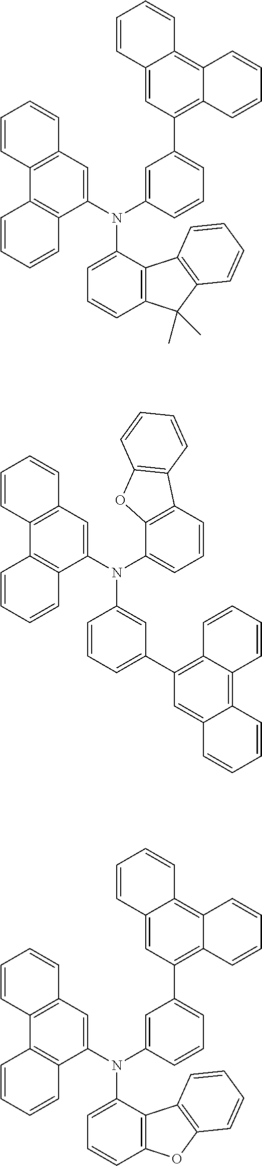 [SMILES: CC1(C)c2ccccc2-c2c(N(c3cccc(-c4cc5ccccc5c5ccccc45)c3)c3cc4ccccc4c4ccccc34)cccc21 ; c1cc(-c2cc3ccccc3c3ccccc23)cc(N(c2cc3ccccc3c3ccccc23)c2cccc3c2oc2ccccc23)c1 ; c1cc(-c2cc3ccccc3c3ccccc23)cc(N(c2cc3ccccc3c3ccccc23)c2cccc3oc4ccccc4c23)c1]

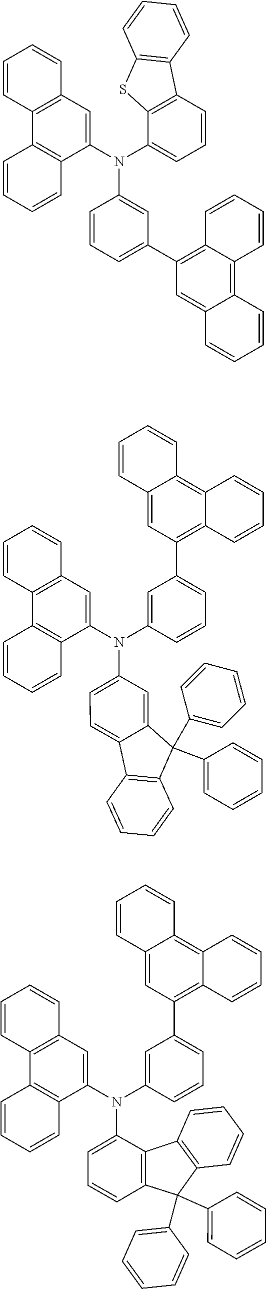 [SMILES: c1cc(-c2cc3ccccc3c3ccccc23)cc(N(c2cc3ccccc3c3ccccc23)c2cccc3c2sc2ccccc23)c1 ; c1ccc(C2(c3ccccc3)c3ccccc3-c3ccc(N(c4cccc(-c5cc6ccccc6c6ccccc56)c4)c4cc5ccccc5c5ccccc45)cc32)cc1 ; c1ccc(C2(c3ccccc3)c3ccccc3-c3c(N(c4cccc(-c5cc6ccccc6c6ccccc56)c4)c4cc5ccccc5c5ccccc45)cccc32)cc1]

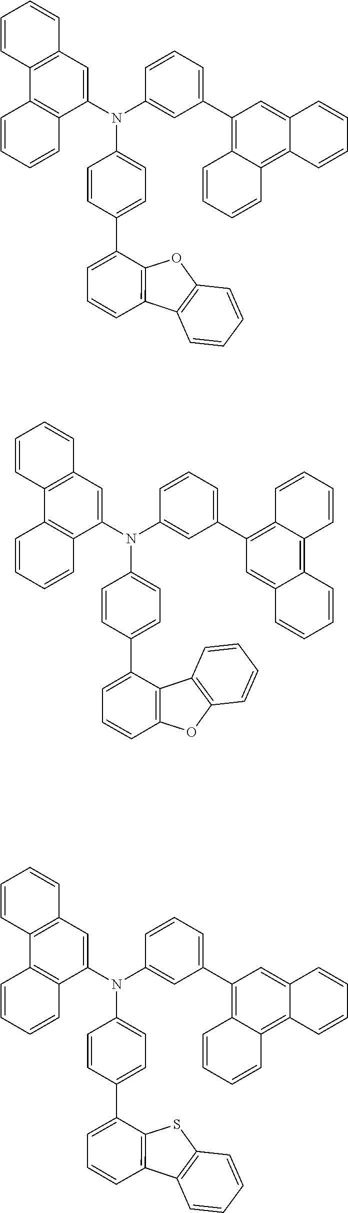 [SMILES: c1cc(-c2cc3ccccc3c3ccccc23)cc(N(c2ccc(-c3cccc4c3oc3ccccc34)cc2)c2cc3ccccc3c3ccccc23)c1 ; c1cc(-c2cc3ccccc3c3ccccc23)cc(N(c2ccc(-c3cccc4oc5ccccc5c34)cc2)c2cc3ccccc3c3ccccc23)c1 ; c1cc(-c2cc3ccccc3c3ccccc23)cc(N(c2ccc(-c3cccc4c3sc3ccccc34)cc2)c2cc3ccccc3c3ccccc23)c1]

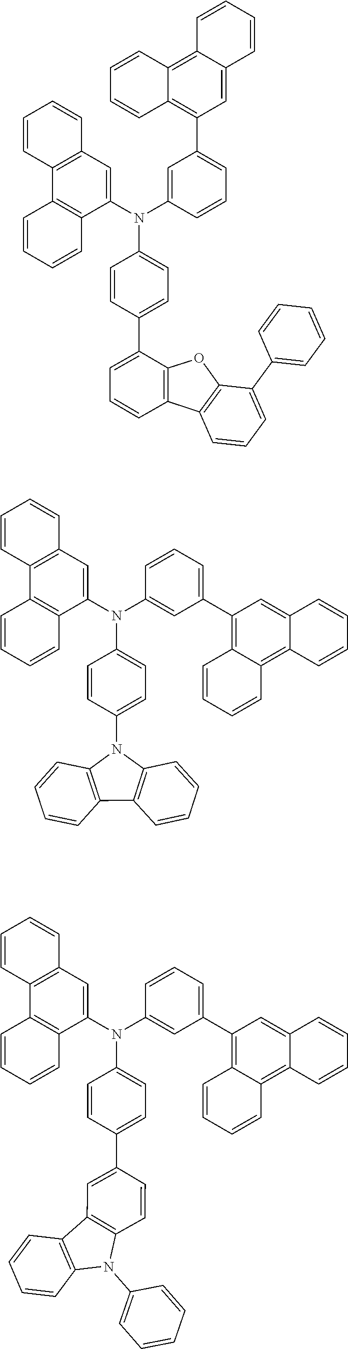 [SMILES: c1ccc(-c2cccc3c2oc2c(-c4ccc(N(c5cccc(-c6cc7ccccc7c7ccccc67)c5)c5cc6ccccc6c6ccccc56)cc4)cccc23)cc1 ; c1cc(-c2cc3ccccc3c3ccccc23)cc(N(c2ccc(-n3c4ccccc4c4ccccc43)cc2)c2cc3ccccc3c3ccccc23)c1 ; c1ccc(-n2c3ccccc3c3cc(-c4ccc(N(c5cccc(-c6cc7ccccc7c7ccccc67)c5)c5cc6ccccc6c6ccccc56)cc4)ccc32)cc1]

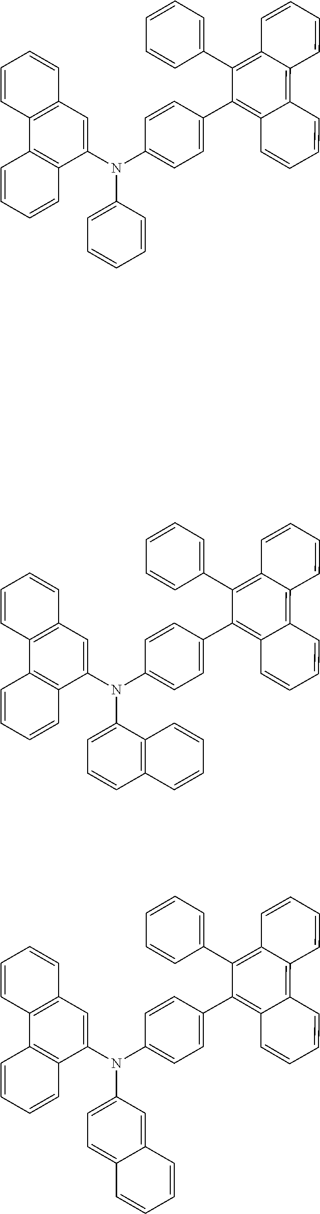 [SMILES: c1ccc(-c2c(-c3ccc(N(c4ccccc4)c4cc5ccccc5c5ccccc45)cc3)c3ccccc3c3ccccc23)cc1 ; c1ccc(-c2c(-c3ccc(N(c4cccc5ccccc45)c4cc5ccccc5c5ccccc45)cc3)c3ccccc3c3ccccc23)cc1 ; c1ccc(-c2c(-c3ccc(N(c4ccc5ccccc5c4)c4cc5ccccc5c5ccccc45)cc3)c3ccccc3c3ccccc23)cc1]

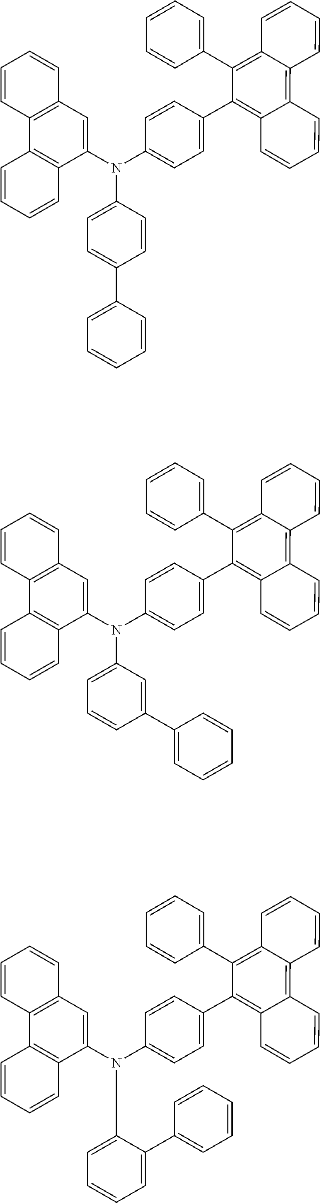 [SMILES: c1ccc(-c2ccc(N(c3ccc(-c4c(-c5ccccc5)c5ccccc5c5ccccc45)cc3)c3cc4ccccc4c4ccccc34)cc2)cc1 ; c1ccc(-c2cccc(N(c3ccc(-c4c(-c5ccccc5)c5ccccc5c5ccccc45)cc3)c3cc4ccccc4c4ccccc34)c2)cc1 ; c1ccc(-c2ccccc2N(c2ccc(-c3c(-c4ccccc4)c4ccccc4c4ccccc34)cc2)c2cc3ccccc3c3ccccc23)cc1]

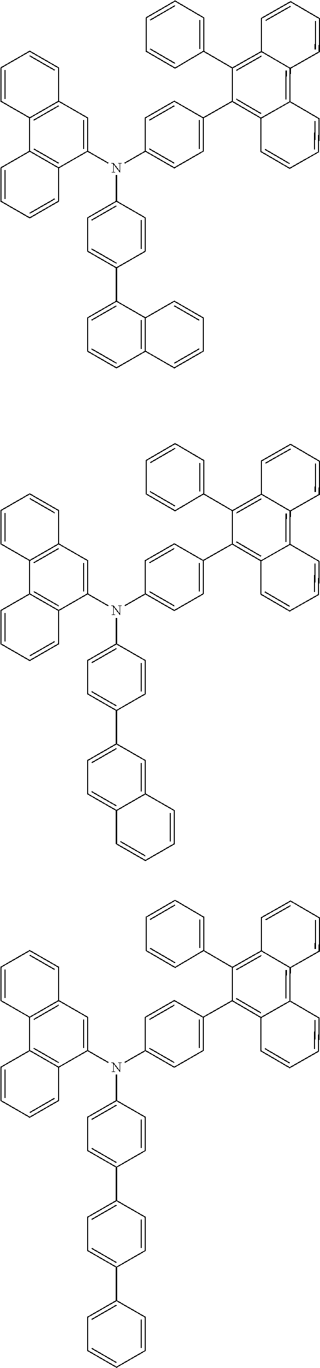 [SMILES: c1ccc(-c2c(-c3ccc(N(c4ccc(-c5cccc6ccccc56)cc4)c4cc5ccccc5c5ccccc45)cc3)c3ccccc3c3ccccc23)cc1 ; c1ccc(-c2c(-c3ccc(N(c4ccc(-c5ccc6ccccc6c5)cc4)c4cc5ccccc5c5ccccc45)cc3)c3ccccc3c3ccccc23)cc1 ; c1ccc(-c2ccc(-c3ccc(N(c4ccc(-c5c(-c6ccccc6)c6ccccc6c6ccccc56)cc4)c4cc5ccccc5c5ccccc45)cc3)cc2)cc1]

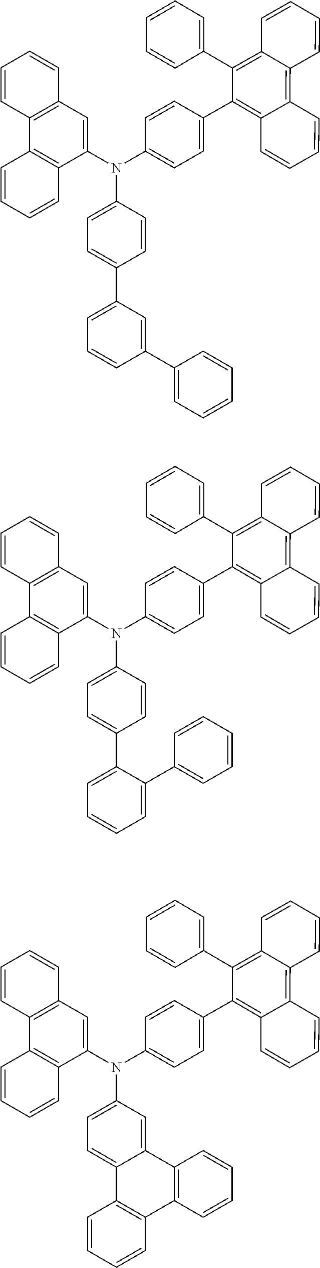 [SMILES: c1ccc(-c2cccc(-c3ccc(N(c4ccc(-c5c(-c6ccccc6)c6ccccc6c6ccccc56)cc4)c4cc5ccccc5c5ccccc45)cc3)c2)cc1 ; c1ccc(-c2ccccc2-c2ccc(N(c3ccc(-c4c(-c5ccccc5)c5ccccc5c5ccccc45)cc3)c3cc4ccccc4c4ccccc34)cc2)cc1 ; c1ccc(-c2c(-c3ccc(N(c4ccc5c6ccccc6c6ccccc6c5c4)c4cc5ccccc5c5ccccc45)cc3)c3ccccc3c3ccccc23)cc1]

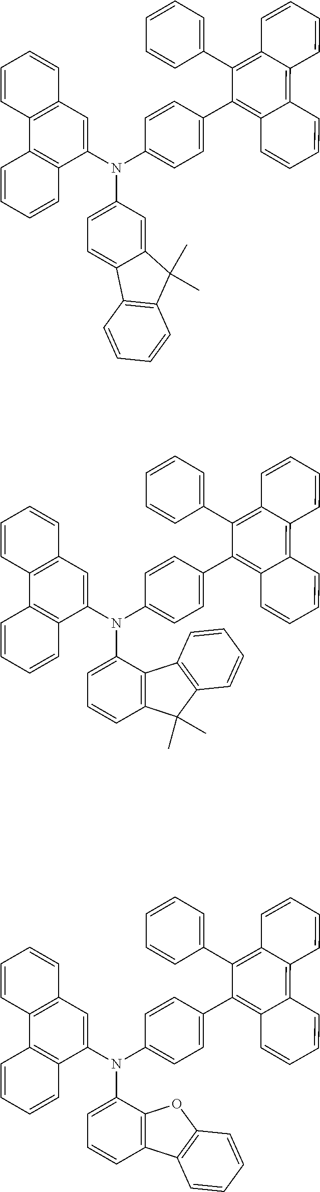 [SMILES: CC1(C)c2ccccc2-c2ccc(N(c3ccc(-c4c(-c5ccccc5)c5ccccc5c5ccccc45)cc3)c3cc4ccccc4c4ccccc34)cc21 ; CC1(C)c2ccccc2-c2c(N(c3ccc(-c4c(-c5ccccc5)c5ccccc5c5ccccc45)cc3)c3cc4ccccc4c4ccccc34)cccc21 ; c1ccc(-c2c(-c3ccc(N(c4cc5ccccc5c5ccccc45)c4cccc5c4oc4ccccc45)cc3)c3ccccc3c3ccccc23)cc1]

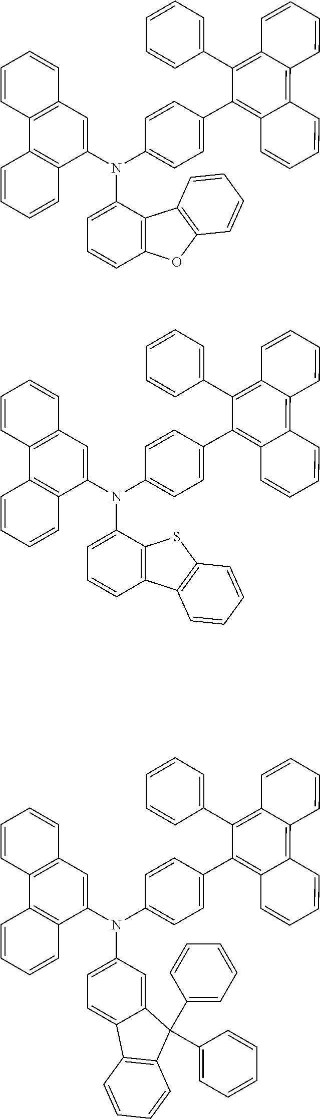 [SMILES: c1ccc(-c2c(-c3ccc(N(c4cc5ccccc5c5ccccc45)c4cccc5oc6ccccc6c45)cc3)c3ccccc3c3ccccc23)cc1 ; c1ccc(-c2c(-c3ccc(N(c4cc5ccccc5c5ccccc45)c4cccc5c4sc4ccccc45)cc3)c3ccccc3c3ccccc23)cc1 ; c1ccc(-c2c(-c3ccc(N(c4ccc5c(c4)C(c4ccccc4)(c4ccccc4)c4ccccc4-5)c4cc5ccccc5c5ccccc45)cc3)c3ccccc3c3ccccc23)cc1]

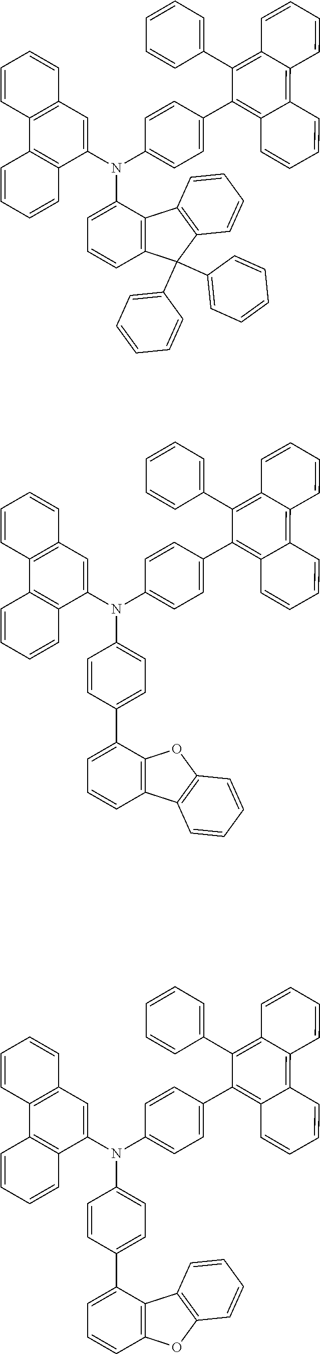 [SMILES: c1ccc(-c2c(-c3ccc(N(c4cccc5c4-c4ccccc4C5(c4ccccc4)c4ccccc4)c4cc5ccccc5c5ccccc45)cc3)c3ccccc3c3ccccc23)cc1 ; c1ccc(-c2c(-c3ccc(N(c4ccc(-c5cccc6c5oc5ccccc56)cc4)c4cc5ccccc5c5ccccc45)cc3)c3ccccc3c3ccccc23)cc1 ; c1ccc(-c2c(-c3ccc(N(c4ccc(-c5cccc6oc7ccccc7c56)cc4)c4cc5ccccc5c5ccccc45)cc3)c3ccccc3c3ccccc23)cc1]

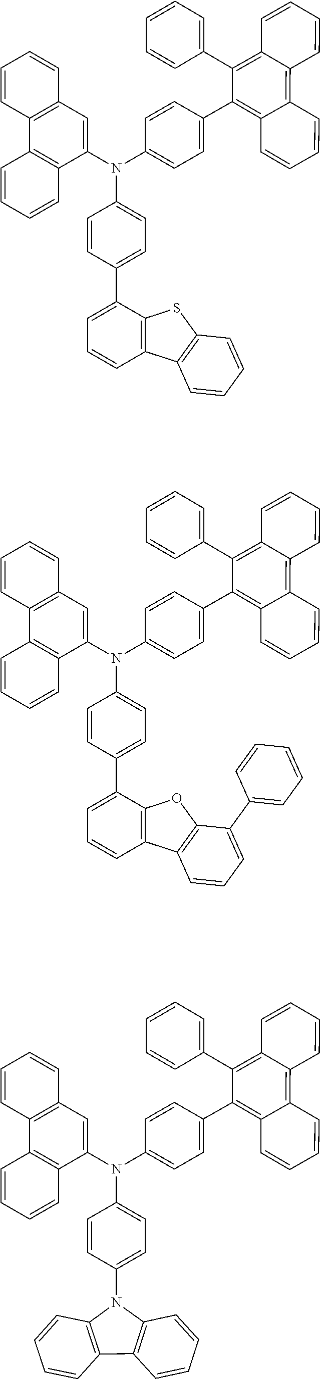 [SMILES: c1ccc(-c2c(-c3ccc(N(c4ccc(-c5cccc6c5sc5ccccc56)cc4)c4cc5ccccc5c5ccccc45)cc3)c3ccccc3c3ccccc23)cc1 ; c1ccc(-c2c(-c3ccc(N(c4ccc(-c5cccc6c5oc5c(-c7ccccc7)cccc56)cc4)c4cc5ccccc5c5ccccc45)cc3)c3ccccc3c3ccccc23)cc1 ; c1ccc(-c2c(-c3ccc(N(c4ccc(-n5c6ccccc6c6ccccc65)cc4)c4cc5ccccc5c5ccccc45)cc3)c3ccccc3c3ccccc23)cc1]

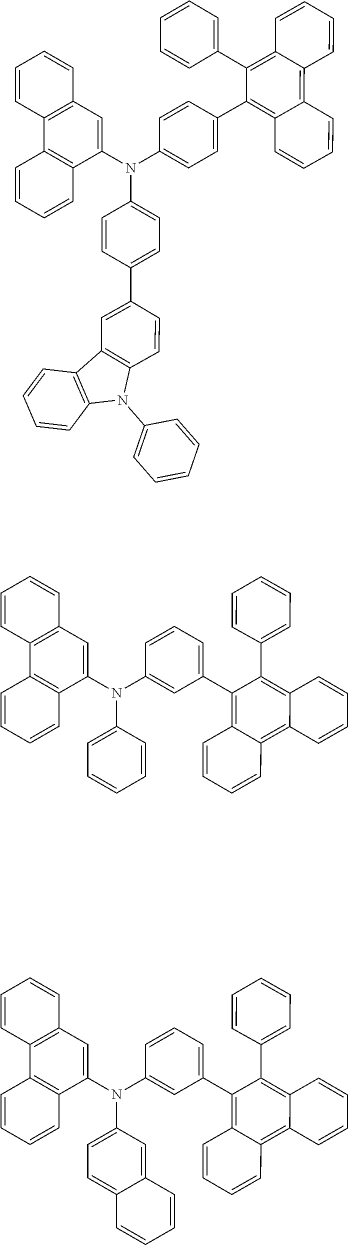 [SMILES: c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1 ; c1ccc(-c2c(-c3cccc(N(c4ccccc4)c4cc5ccccc5c5ccccc45)c3)c3ccccc3c3ccccc23)cc1 ; c1ccc(-c2c(-c3cccc(N(c4ccc5ccccc5c4)c4cc5ccccc5c5ccccc45)c3)c3ccccc3c3ccccc23)cc1]

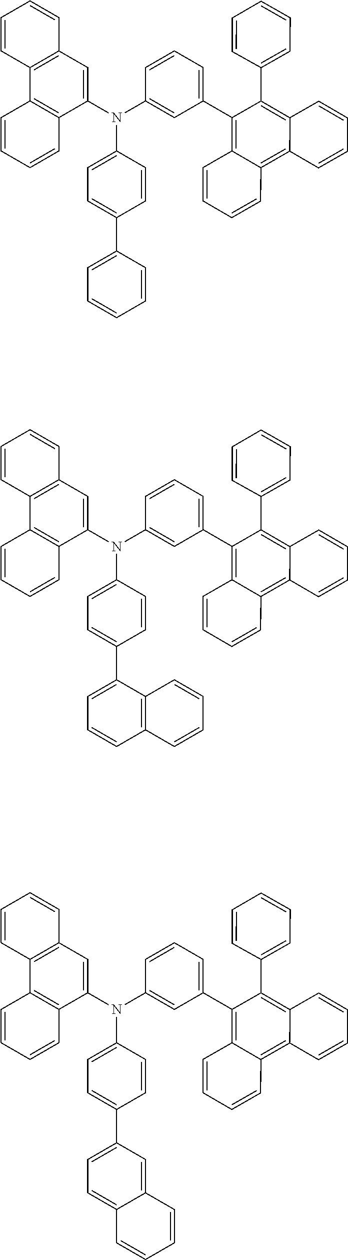 [SMILES: c1ccc(-c2ccc(N(c3cccc(-c4c(-c5ccccc5)c5ccccc5c5ccccc45)c3)c3cc4ccccc4c4ccccc34)cc2)cc1 ; c1ccc(-c2c(-c3cccc(N(c4ccc(-c5cccc6ccccc56)cc4)c4cc5ccccc5c5ccccc45)c3)c3ccccc3c3ccccc23)cc1 ; c1ccc(-c2c(-c3cccc(N(c4ccc(-c5ccc6ccccc6c5)cc4)c4cc5ccccc5c5ccccc45)c3)c3ccccc3c3ccccc23)cc1]

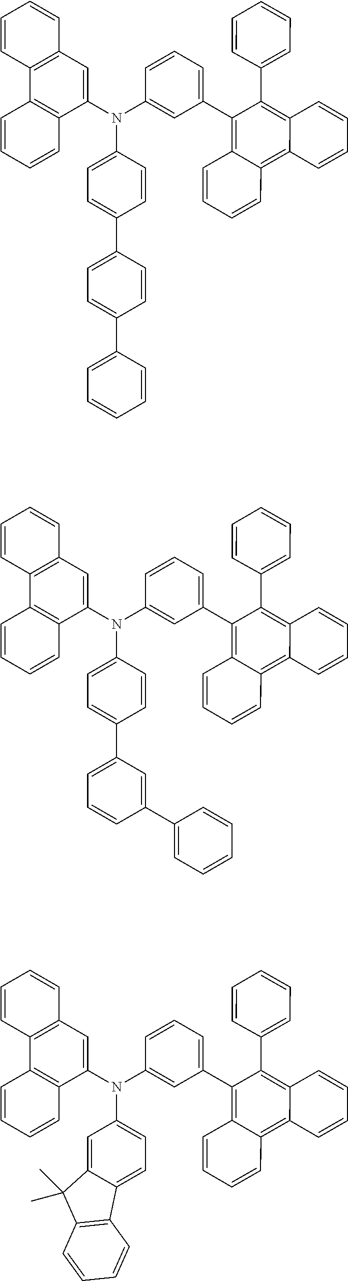 [SMILES: c1ccc(-c2ccc(-c3ccc(N(c4cccc(-c5c(-c6ccccc6)c6ccccc6c6ccccc56)c4)c4cc5ccccc5c5ccccc45)cc3)cc2)cc1 ; c1ccc(-c2cccc(-c3ccc(N(c4cccc(-c5c(-c6ccccc6)c6ccccc6c6ccccc56)c4)c4cc5ccccc5c5ccccc45)cc3)c2)cc1 ; CC1(C)c2ccccc2-c2ccc(N(c3cccc(-c4c(-c5ccccc5)c5ccccc5c5ccccc45)c3)c3cc4ccccc4c4ccccc34)cc21]

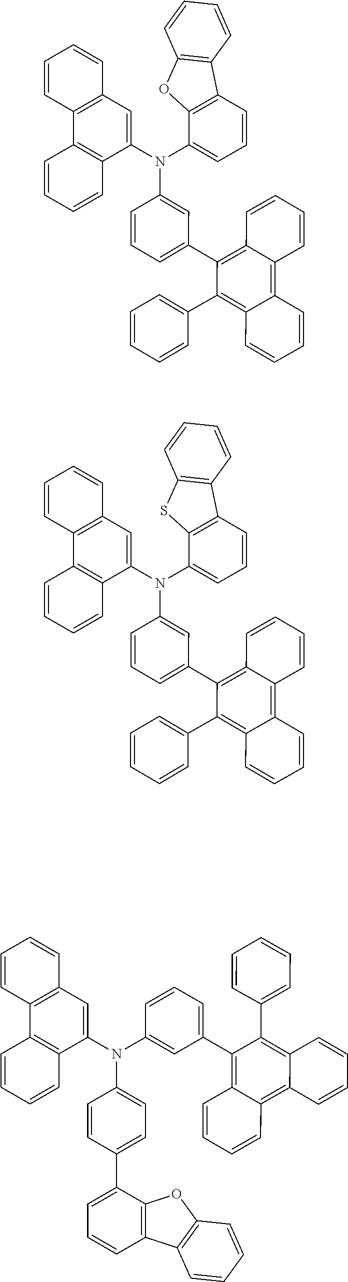 [SMILES: c1ccc(-c2c(-c3cccc(N(c4cc5ccccc5c5ccccc45)c4cccc5c4oc4ccccc45)c3)c3ccccc3c3ccccc23)cc1 ; c1ccc(-c2c(-c3cccc(N(c4cc5ccccc5c5ccccc45)c4cccc5c4sc4ccccc45)c3)c3ccccc3c3ccccc23)cc1 ; c1ccc(-c2c(-c3cccc(N(c4ccc(-c5cccc6c5oc5ccccc56)cc4)c4cc5ccccc5c5ccccc45)c3)c3ccccc3c3ccccc23)cc1]

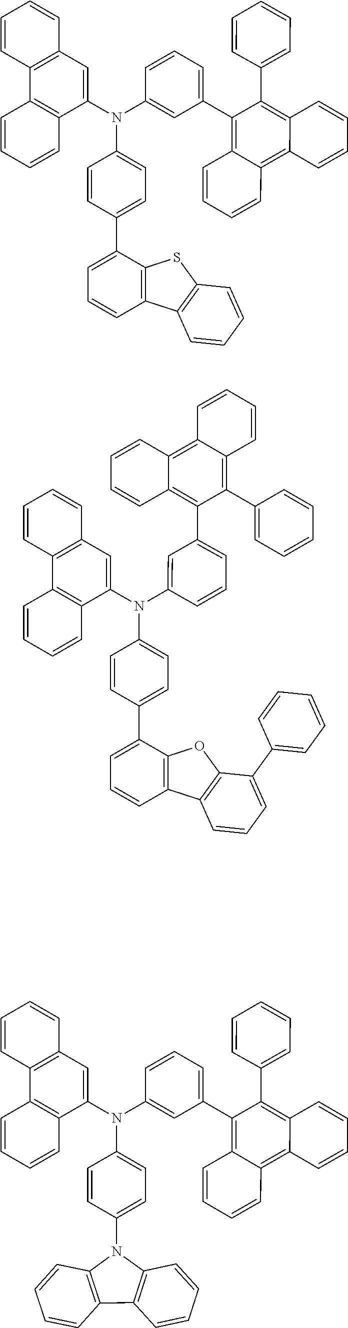

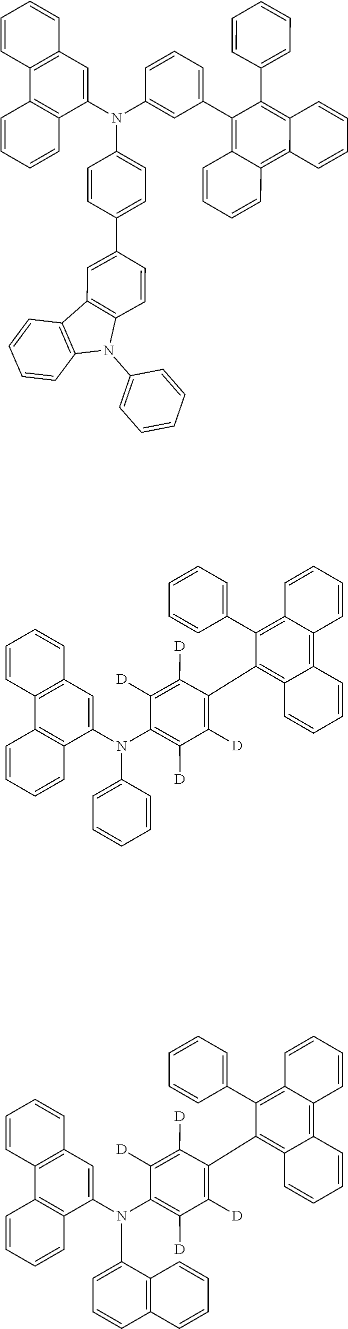 [SMILES: c1ccc(-c2c(-c3cccc(N(c4ccc(-c5ccc6c(c5)c5ccccc5n6-c5ccccc5)cc4)c4cc5ccccc5c5ccccc45)c3)c3ccccc3c3ccccc23)cc1 ; [2H]c1c([2H])c(N(c2ccccc2)c2cc3ccccc3c3ccccc23)c([2H])c([2H])c1-c1c(-c2ccccc2)c2ccccc2c2ccccc12 ; [2H]c1c([2H])c(N(c2cccc3ccccc23)c2cc3ccccc3c3ccccc23)c([2H])c([2H])c1-c1c(-c2ccccc2)c2ccccc2c2ccccc12]

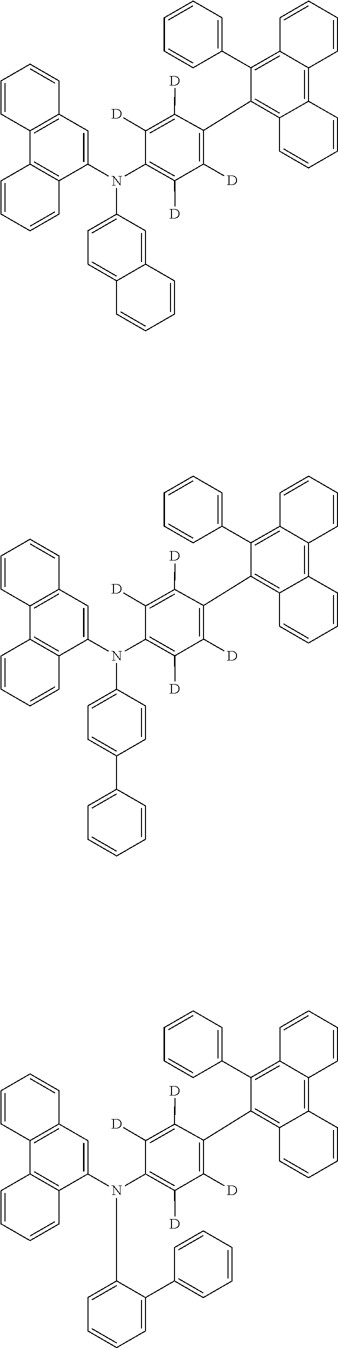 [SMILES: [2H]c1c([2H])c(N(c2ccc3ccccc3c2)c2cc3ccccc3c3ccccc23)c([2H])c([2H])c1-c1c(-c2ccccc2)c2ccccc2c2ccccc12 ; [2H]c1c([2H])c(N(c2ccc(-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccccc2-c2ccccc2)c2cc3ccccc3c3ccccc23)c([2H])c([2H])c1-c1c(-c2ccccc2)c2ccccc2c2ccccc12]

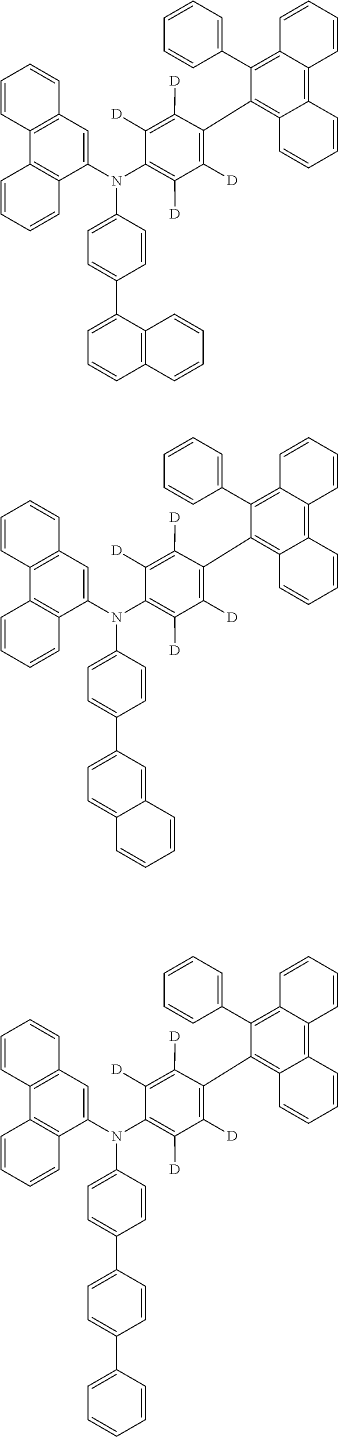 [SMILES: [2H]c1c([2H])c(N(c2ccc(-c3cccc4ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc4ccccc4c3)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc(-c4ccccc4)cc3)cc2)c2cc3ccccc3c3ccccc23)c([2H])c([2H])c1-c1c(-c2ccccc2)c2ccccc2c2ccccc12]

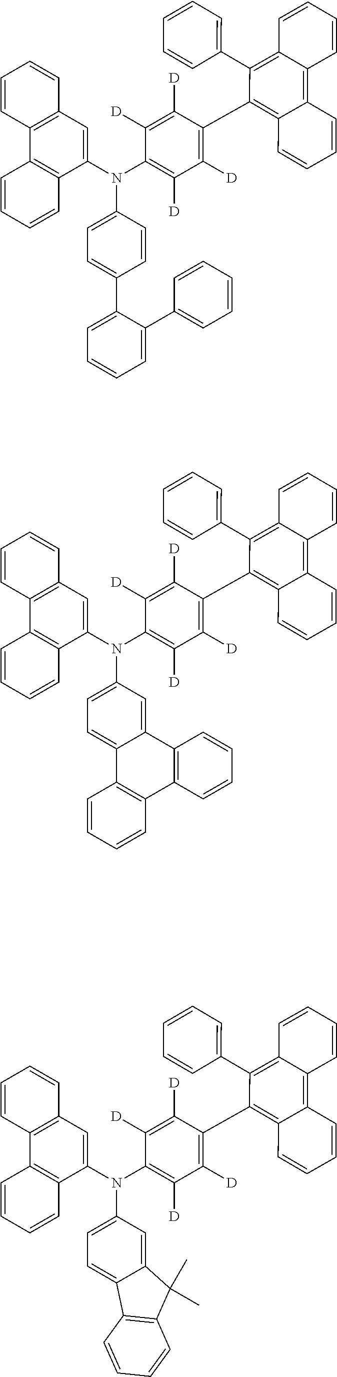 [SMILES: [2H]c1c([2H])c(N(c2ccc(-c3ccccc3-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccc3c4ccccc4c4ccccc4c3c2)c2cc3ccccc3c3ccccc23)c([2H])c([2H])c1-c1c(-c2ccccc2)c2ccccc2c2ccccc12 ; [2H]c1c([2H])c(N(c2ccc3c(c2)C(C)(C)c2ccccc2-3)c2cc3ccccc3c3ccccc23)c([2H])c([2H])c1-c1c(-c2ccccc2)c2ccccc2c2ccccc12]

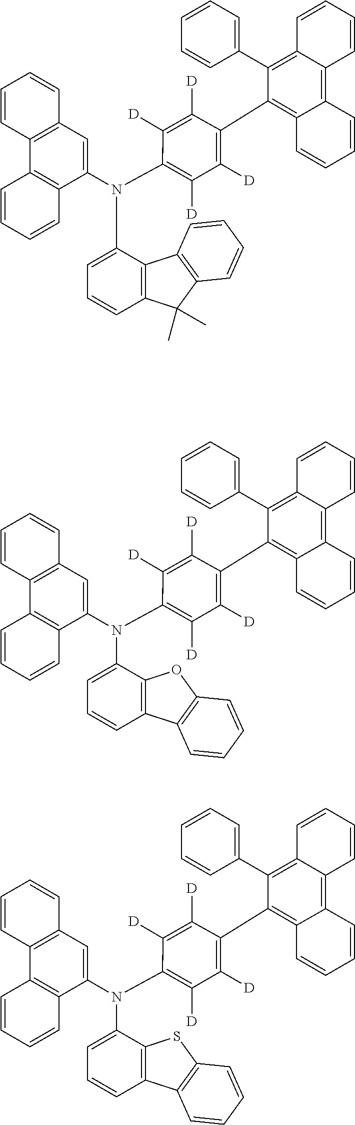 [SMILES: [2H]c1c([2H])c(N(c2cccc3c2-c2ccccc2C3(C)C)c2cc3ccccc3c3ccccc23)c([2H])c([2H])c1-c1c(-c2ccccc2)c2ccccc2c2ccccc12 ; [2H]c1c([2H])c(N(c2cc3ccccc3c3ccccc23)c2cccc3c2oc2ccccc23)c([2H])c([2H])c1-c1c(-c2ccccc2)c2ccccc2c2ccccc12 ; [2H]c1c([2H])c(N(c2cc3ccccc3c3ccccc23)c2cccc3c2sc2ccccc23)c([2H])c([2H])c1-c1c(-c2ccccc2)c2ccccc2c2ccccc12]

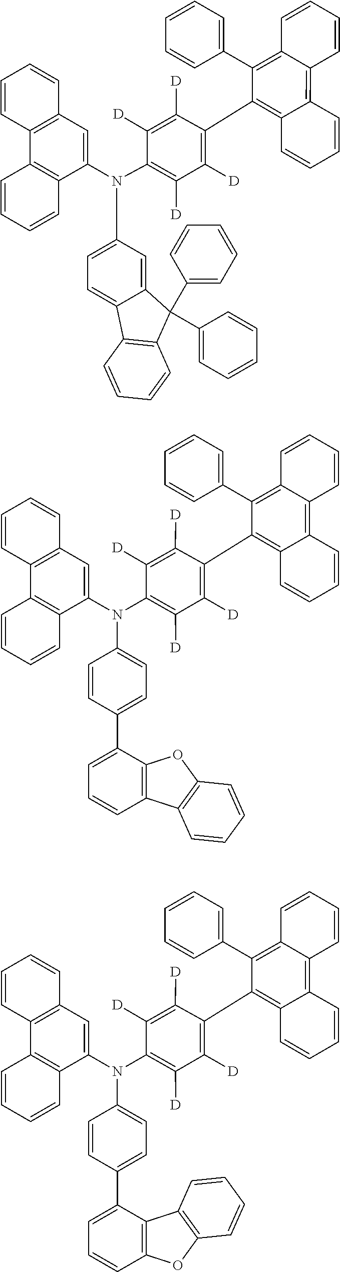 [SMILES: [2H]c1c([2H])c(N(c2ccc3c(c2)C(c2ccccc2)(c2ccccc2)c2ccccc2-3)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3oc3ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4oc5ccccc5c34)cc2)c2cc3ccccc3c3ccccc23)c([2H])c([2H])c1-c1c(-c2ccccc2)c2ccccc2c2ccccc12]

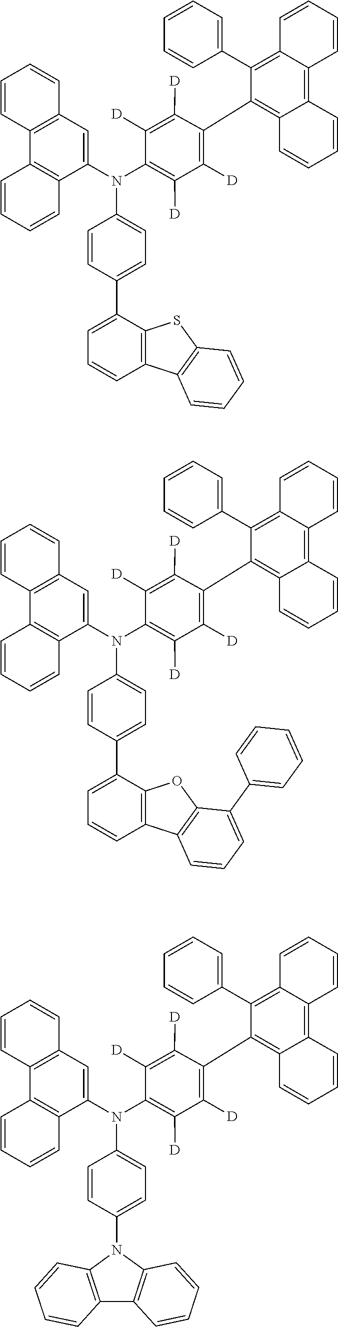 [SMILES: [2H]c1c([2H])c(N(c2ccc(-c3cccc4c3sc3ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3oc3c(-c5ccccc5)cccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-n3c4ccccc4c4ccccc43)cc2)c2cc3ccccc3c3ccccc23)c([2H])c([2H])c1-c1c(-c2ccccc2)c2ccccc2c2ccccc12]

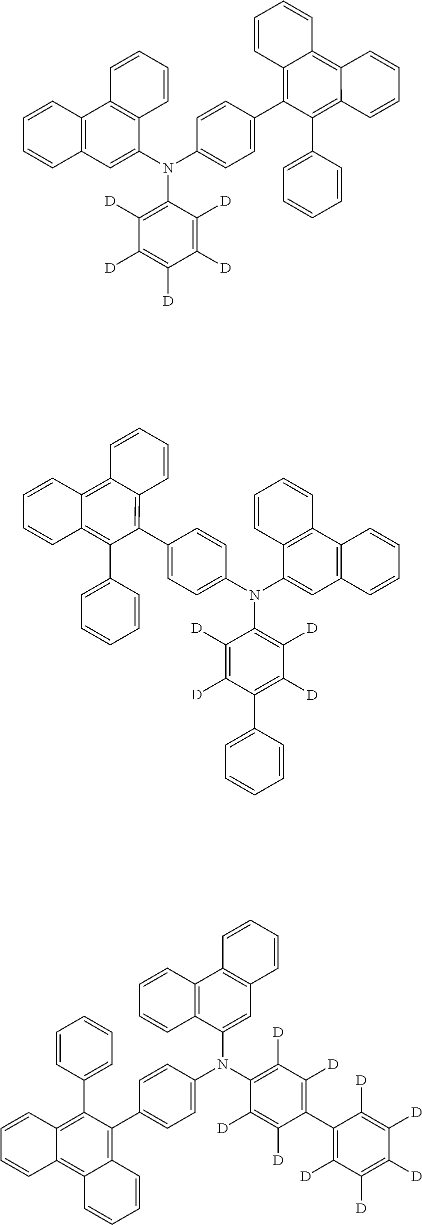 [SMILES: [2H]c1c([2H])c([2H])c(N(c2ccc(-c3c(-c4ccccc4)c4ccccc4c4ccccc34)cc2)c2cc3ccccc3c3ccccc23)c([2H])c1[2H] ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1 ; [2H]c1c([2H])c([2H])c(-c2c([2H])c([2H])c(N(c3ccc(-c4c(-c5ccccc5)c5ccccc5c5ccccc45)cc3)c3cc4ccccc4c4ccccc34)c([2H])c2[2H])c([2H])c1[2H]]

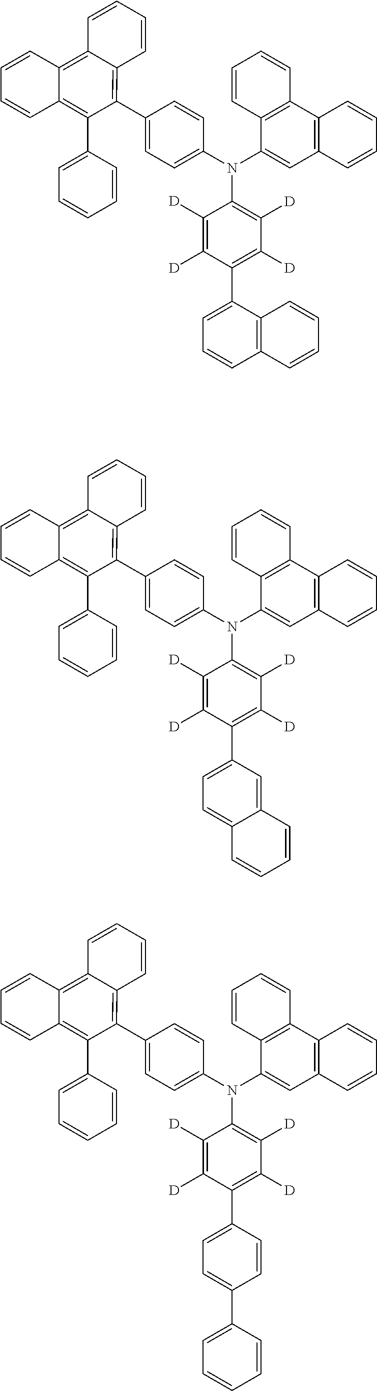 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc2ccccc2c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc(-c2ccccc2)cc1]

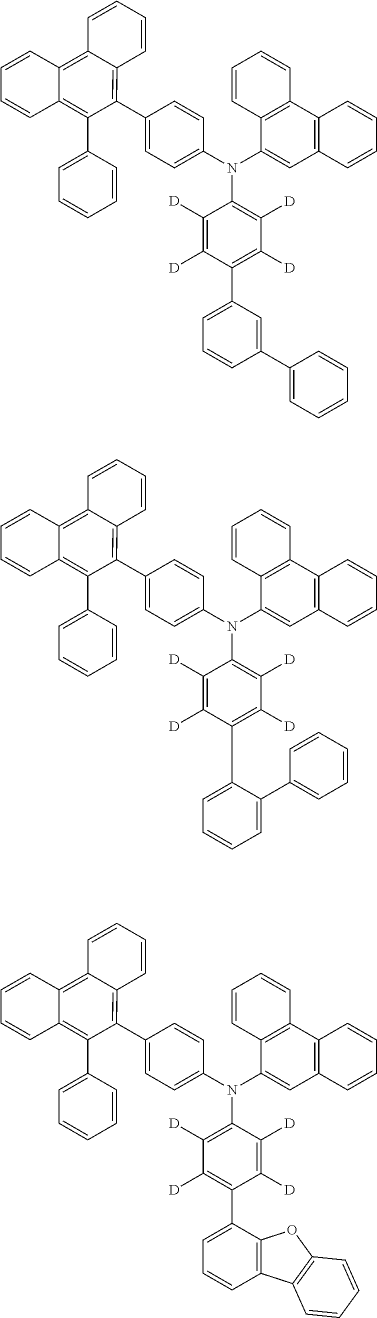 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc(-c2ccccc2)c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1-c1ccccc1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1oc1ccccc12]

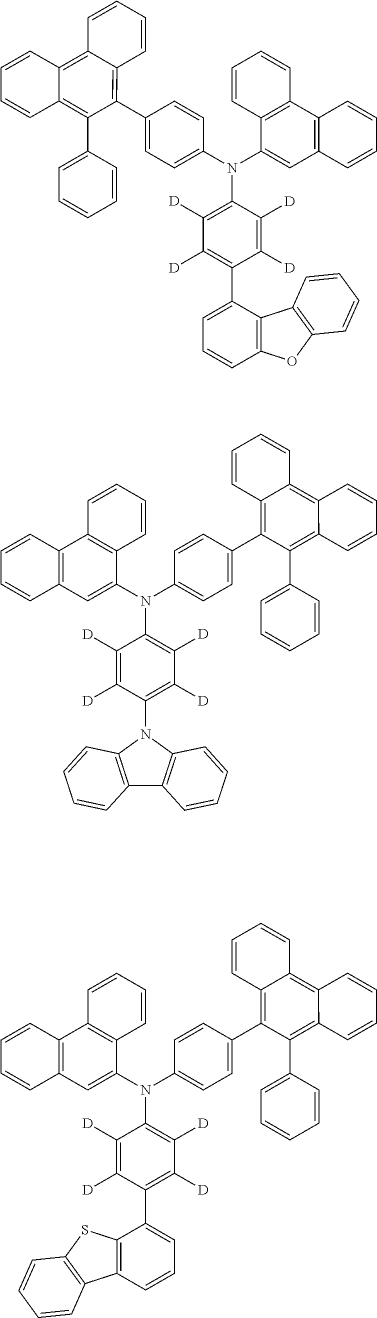 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2oc3ccccc3c12 ; [2H]c1c([2H])c(-n2c3ccccc3c3ccccc32)c([2H])c([2H])c1N(c1ccc(-c2c(-c3ccccc3)c3ccccc3c3ccccc23)cc1)c1cc2ccccc2c2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1sc1ccccc12]

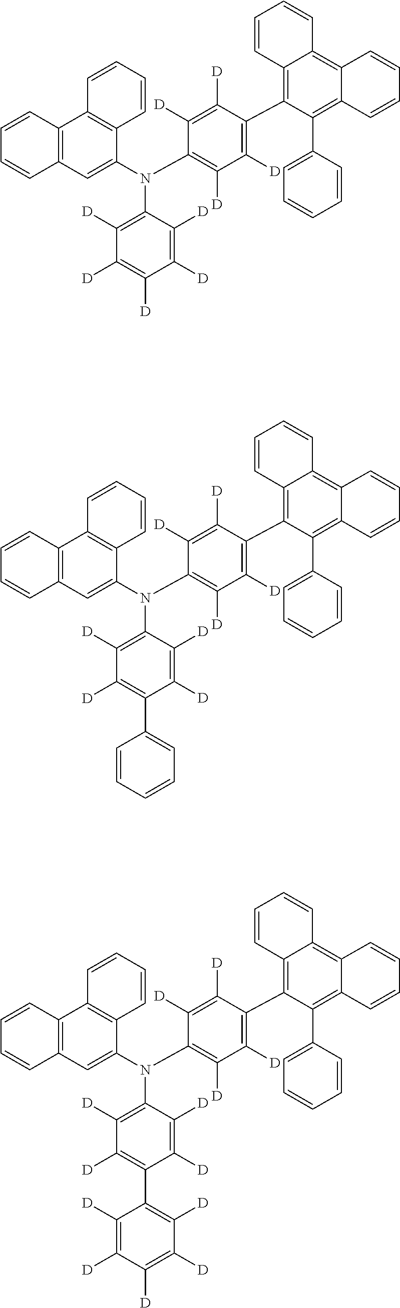 [SMILES: [2H]c1c([2H])c([2H])c(N(c2c([2H])c([2H])c(-c3c(-c4ccccc4)c4ccccc4c4ccccc34)c([2H])c2[2H])c2cc3ccccc3c3ccccc23)c([2H])c1[2H] ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1 ; [2H]c1c([2H])c([2H])c(-c2c([2H])c([2H])c(N(c3c([2H])c([2H])c(-c4c(-c5ccccc5)c5ccccc5c5ccccc45)c([2H])c3[2H])c3cc4ccccc4c4ccccc34)c([2H])c2[2H])c([2H])c1[2H]]

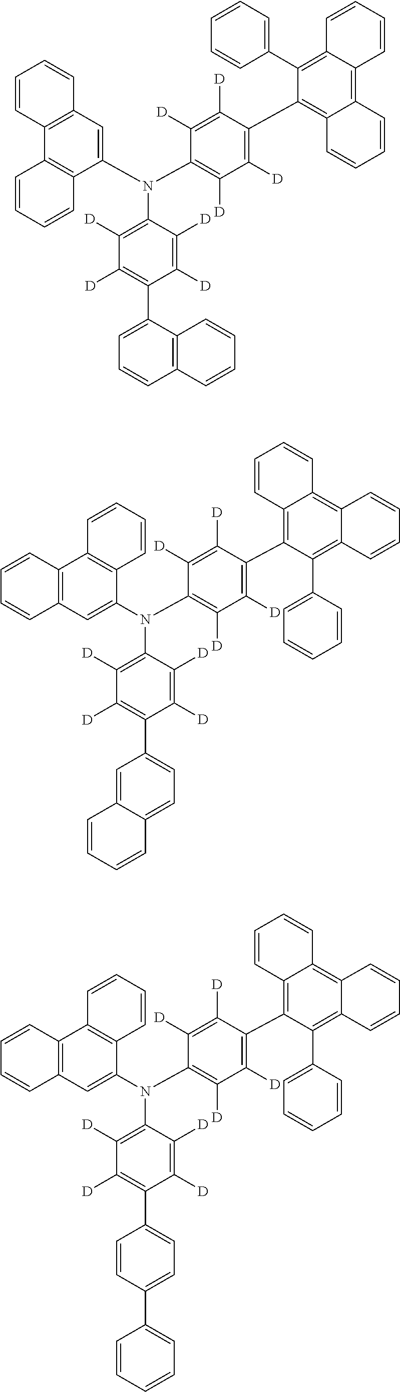 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1cccc2ccccc12 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc(-c2ccccc2)cc1]

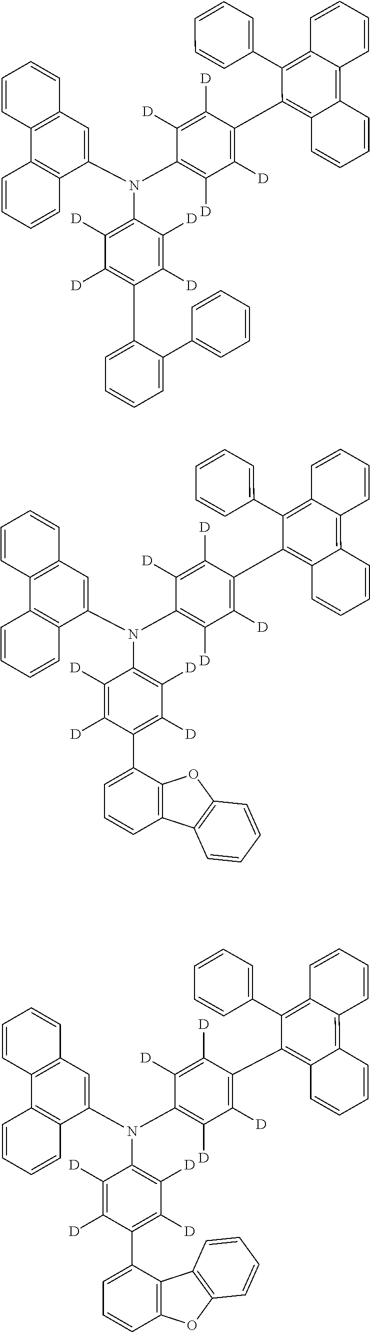 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1-c1ccccc1 ; [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1c(-c2ccccc2)c2ccccc2c2ccccc12]

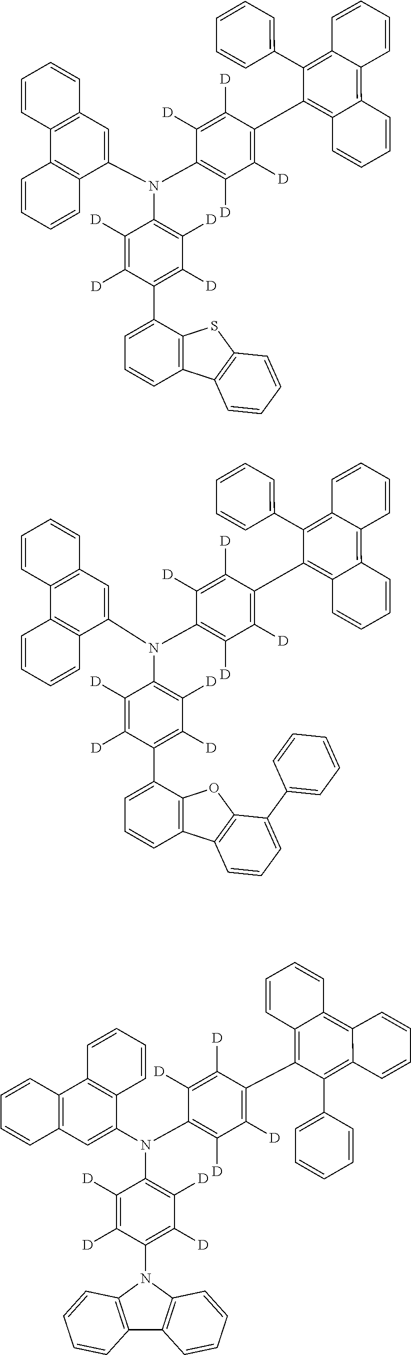 [SMILES: [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-n3c4ccccc4c4ccccc43)c([2H])c2[2H])c2cc3ccccc3c3ccccc23)c([2H])c([2H])c1-c1c(-c2ccccc2)c2ccccc2c2ccccc12]

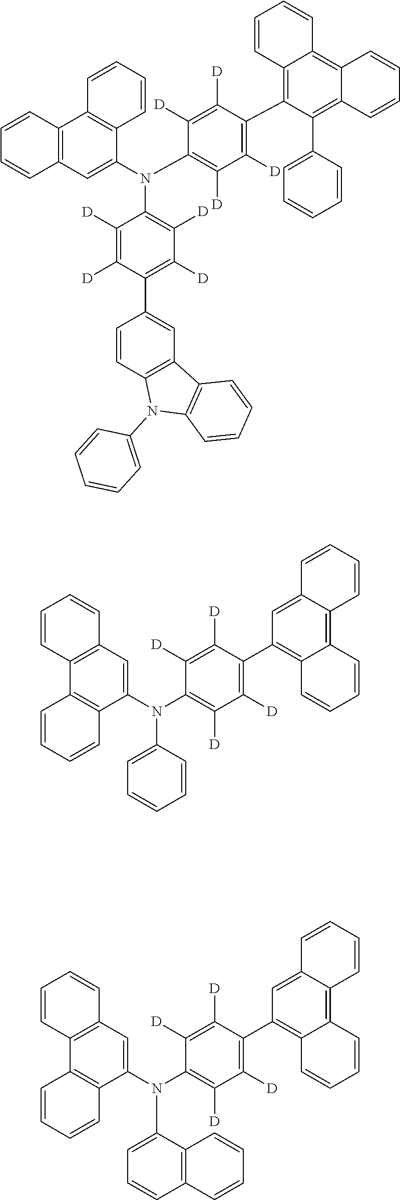

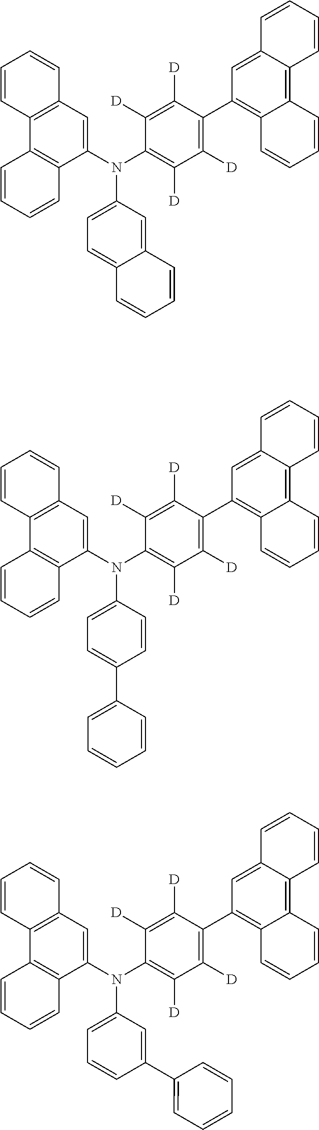

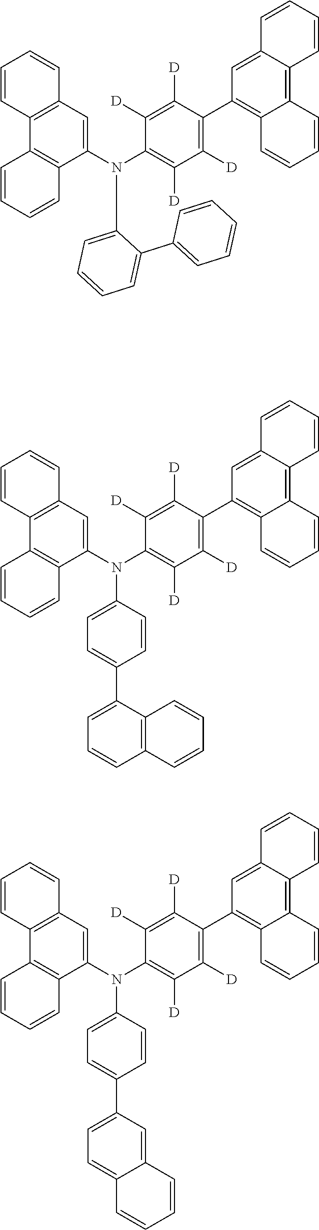

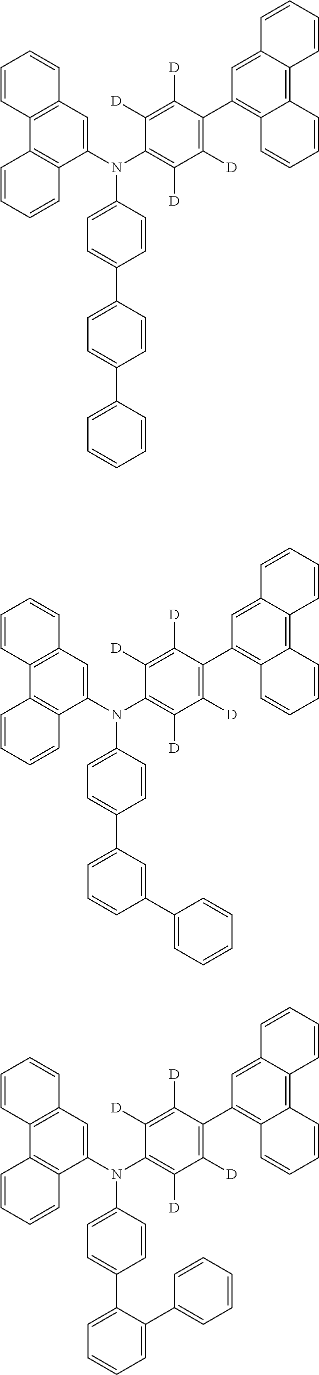

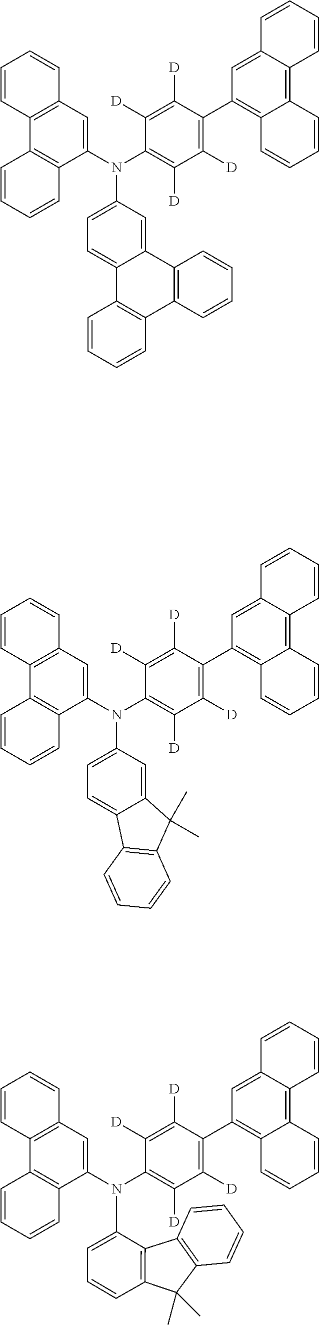

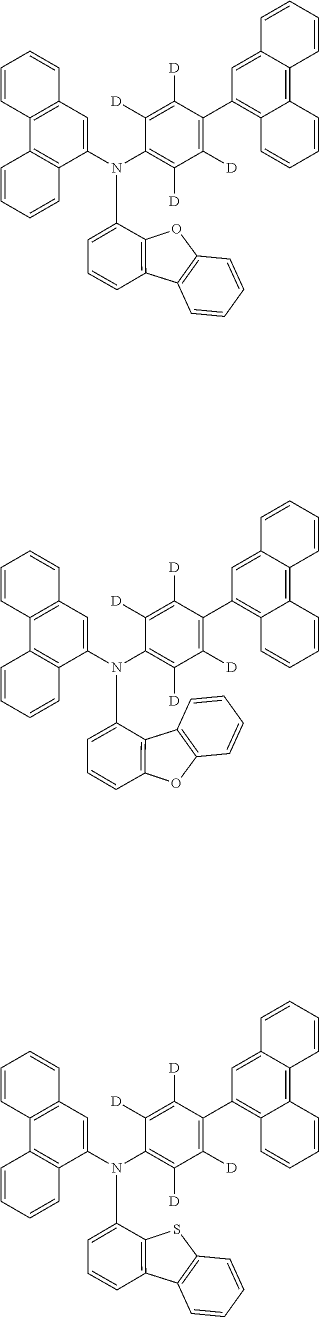 [SMILES: [2H]c1c([2H])c(N(c2cc3ccccc3c3ccccc23)c2cccc3c2oc2ccccc23)c([2H])c([2H])c1-c1cc2ccccc2c2ccccc12 ; [2H]c1c([2H])c(N(c2cc3ccccc3c3ccccc23)c2cccc3oc4ccccc4c23)c([2H])c([2H])c1-c1cc2ccccc2c2ccccc12 ; [2H]c1c([2H])c(N(c2cc3ccccc3c3ccccc23)c2cccc3c2sc2ccccc23)c([2H])c([2H])c1-c1cc2ccccc2c2ccccc12]

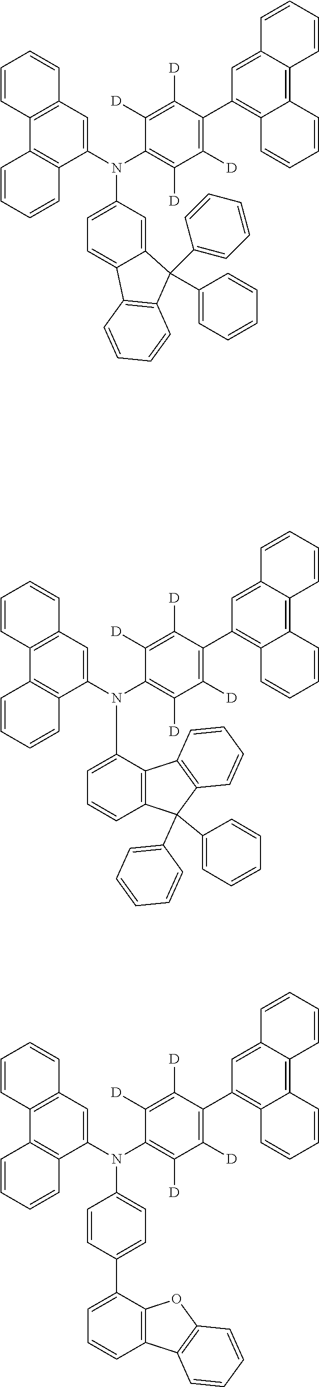

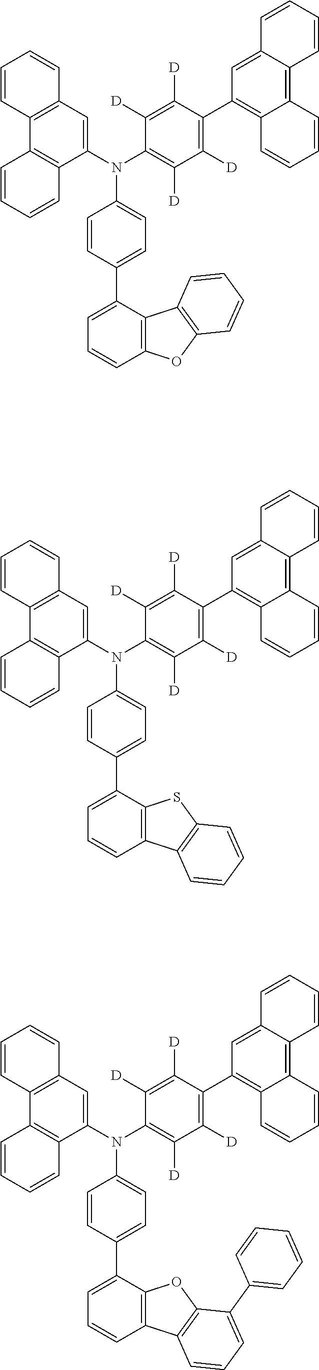

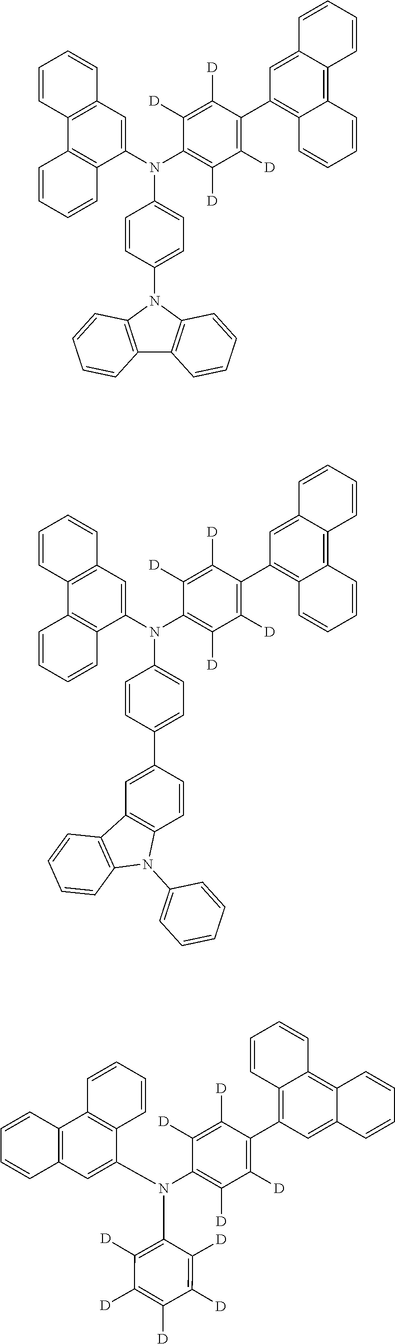

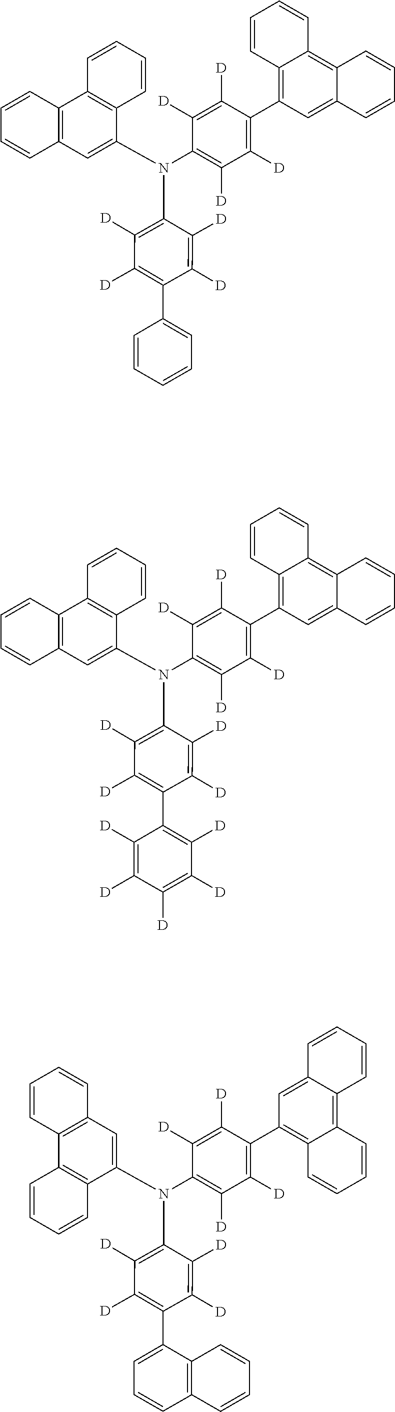

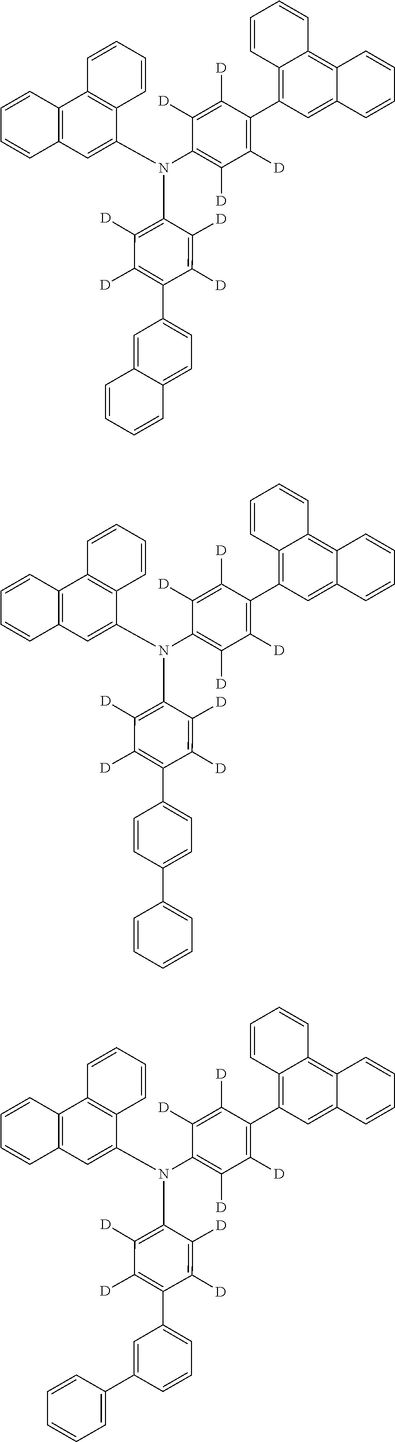 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc(-c2ccccc2)cc1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc(-c2ccccc2)c1]

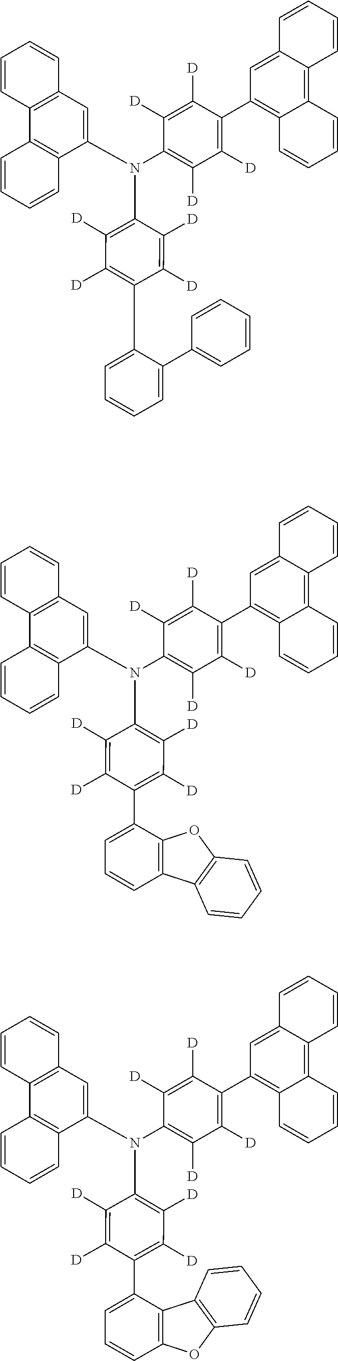

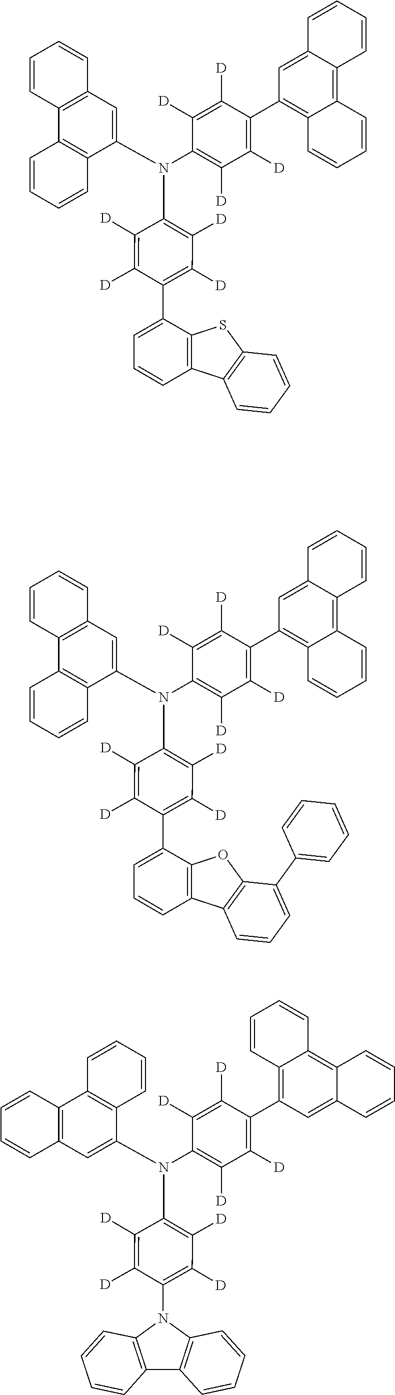

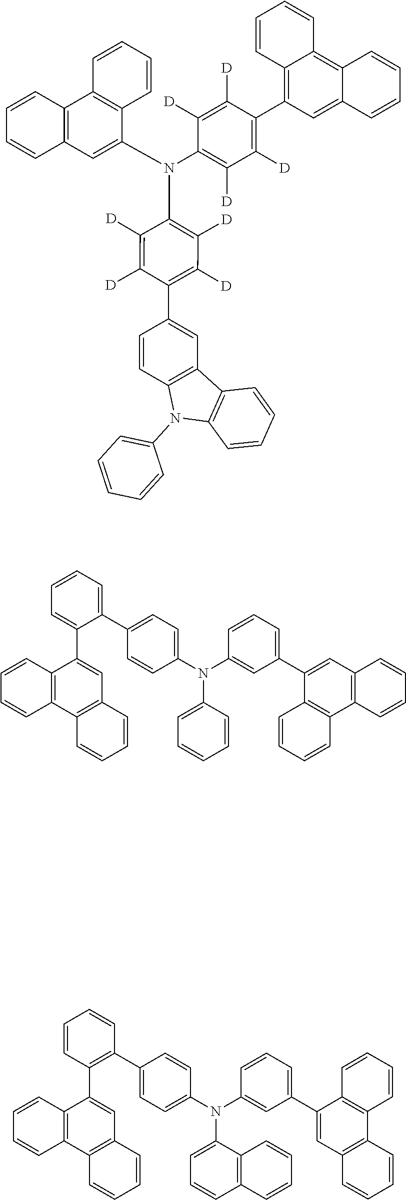

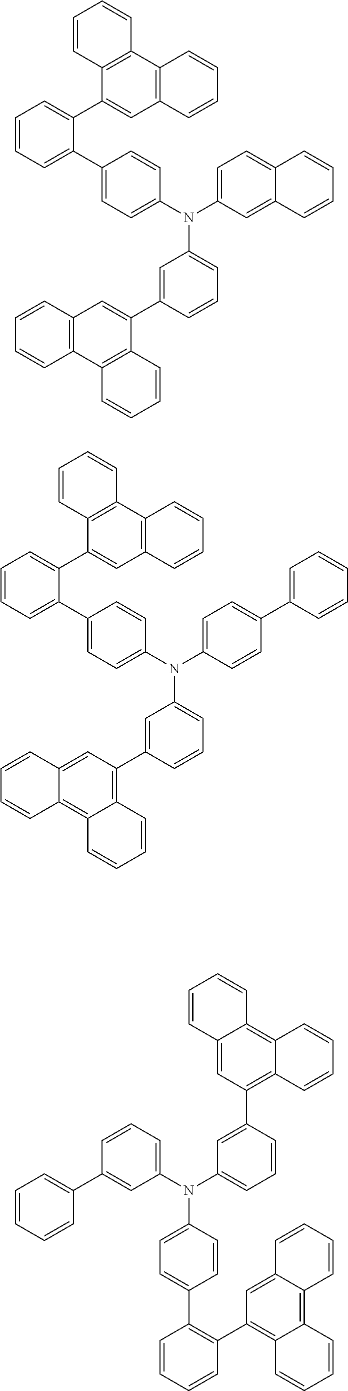 [SMILES: c1cc(-c2cc3ccccc3c3ccccc23)cc(N(c2ccc(-c3ccccc3-c3cc4ccccc4c4ccccc34)cc2)c2ccc3ccccc3c2)c1 ; c1ccc(-c2ccc(N(c3ccc(-c4ccccc4-c4cc5ccccc5c5ccccc45)cc3)c3cccc(-c4cc5ccccc5c5ccccc45)c3)cc2)cc1 ; c1ccc(-c2cccc(N(c3ccc(-c4ccccc4-c4cc5ccccc5c5ccccc45)cc3)c3cccc(-c4cc5ccccc5c5ccccc45)c3)c2)cc1]

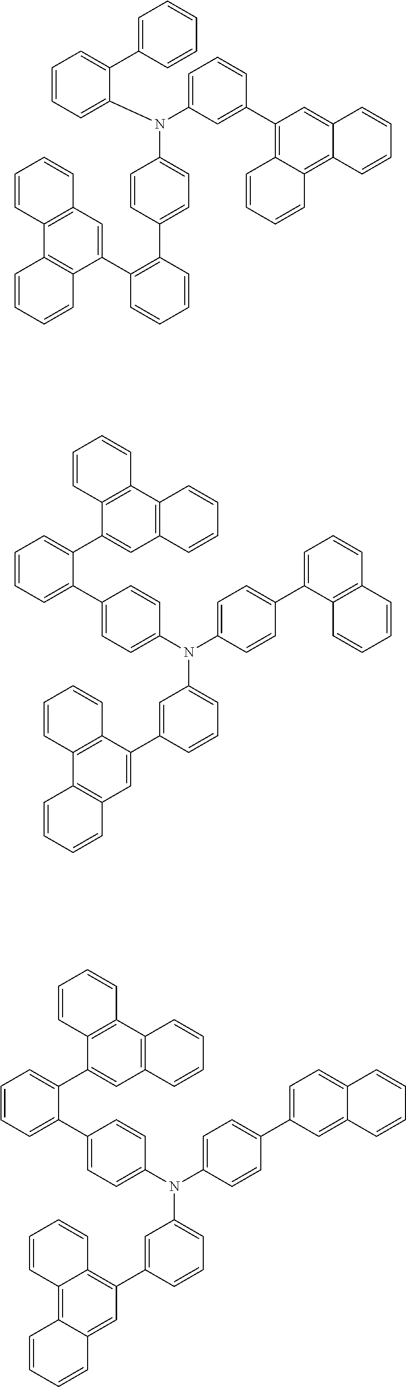 [SMILES: c1ccc(-c2ccccc2N(c2ccc(-c3ccccc3-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3ccccc3-c3cc4ccccc4c4ccccc34)cc2)c2ccc(-c3cccc4ccccc34)cc2)c1 ; c1cc(-c2cc3ccccc3c3ccccc23)cc(N(c2ccc(-c3ccc4ccccc4c3)cc2)c2ccc(-c3ccccc3-c3cc4ccccc4c4ccccc34)cc2)c1]

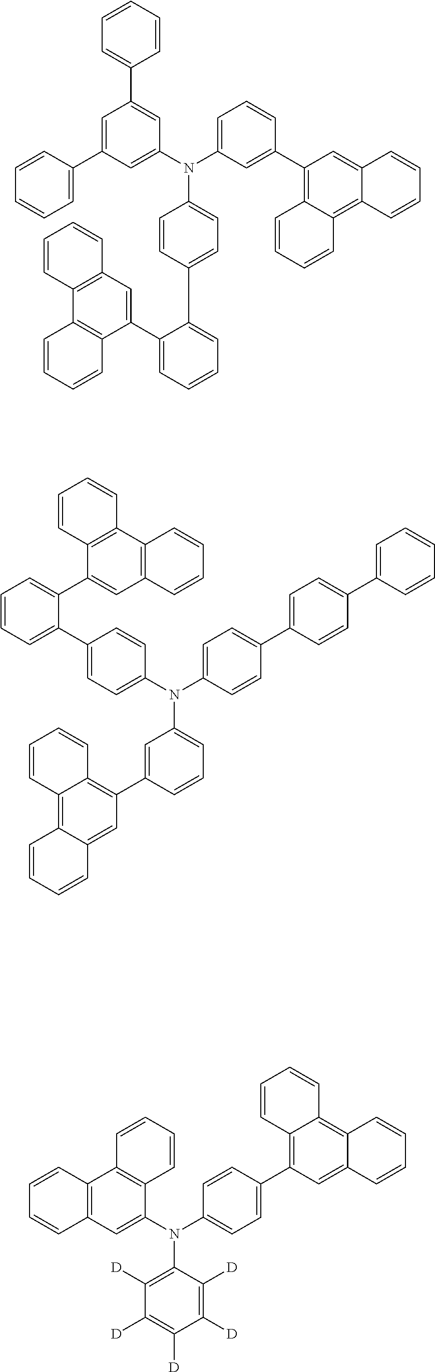 [SMILES: c1ccc(-c2cc(-c3ccccc3)cc(N(c3ccc(-c4ccccc4-c4cc5ccccc5c5ccccc45)cc3)c3cccc(-c4cc5ccccc5c5ccccc45)c3)c2)cc1 ; c1ccc(-c2ccc(-c3ccc(N(c4ccc(-c5ccccc5-c5cc6ccccc6c6ccccc56)cc4)c4cccc(-c5cc6ccccc6c6ccccc56)c4)cc3)cc2)cc1 ; [2H]c1c([2H])c([2H])c(N(c2ccc(-c3cc4ccccc4c4ccccc34)cc2)c2cc3ccccc3c3ccccc23)c([2H])c1[2H]]

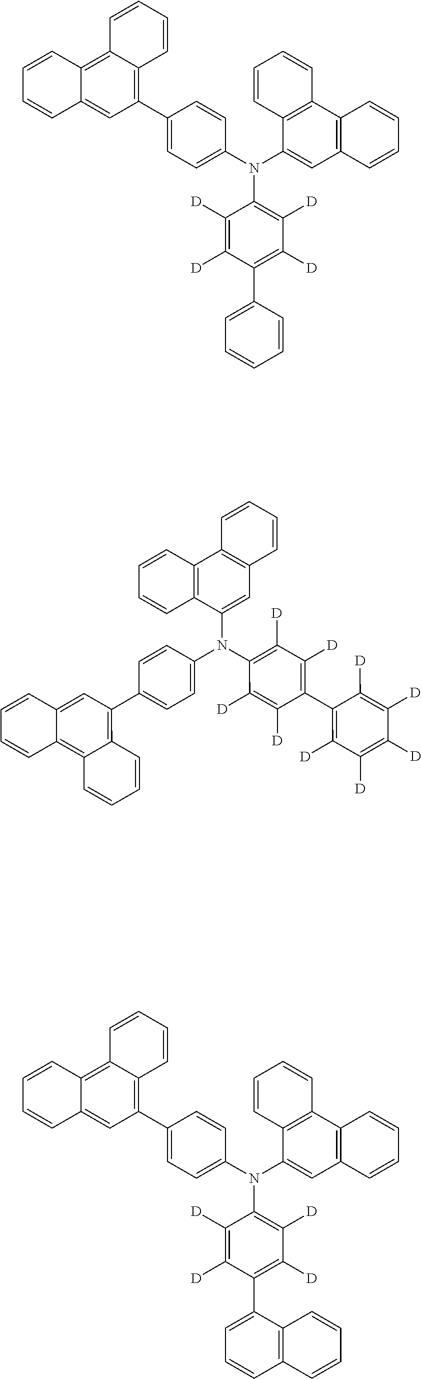 [SMILES: [2H]c1c([2H])c(N(c2ccc(-c3cc4ccccc4c4ccccc34)cc2)c2cc3ccccc3c3ccccc23)c([2H])c([2H])c1-c1ccccc1 ; [2H]c1c([2H])c([2H])c(-c2c([2H])c([2H])c(N(c3ccc(-c4cc5ccccc5c5ccccc45)cc3)c3cc4ccccc4c4ccccc34)c([2H])c2[2H])c([2H])c1[2H] ; [2H]c1c([2H])c(N(c2ccc(-c3cc4ccccc4c4ccccc34)cc2)c2cc3ccccc3c3ccccc23)c([2H])c([2H])c1-c1cccc2ccccc12]

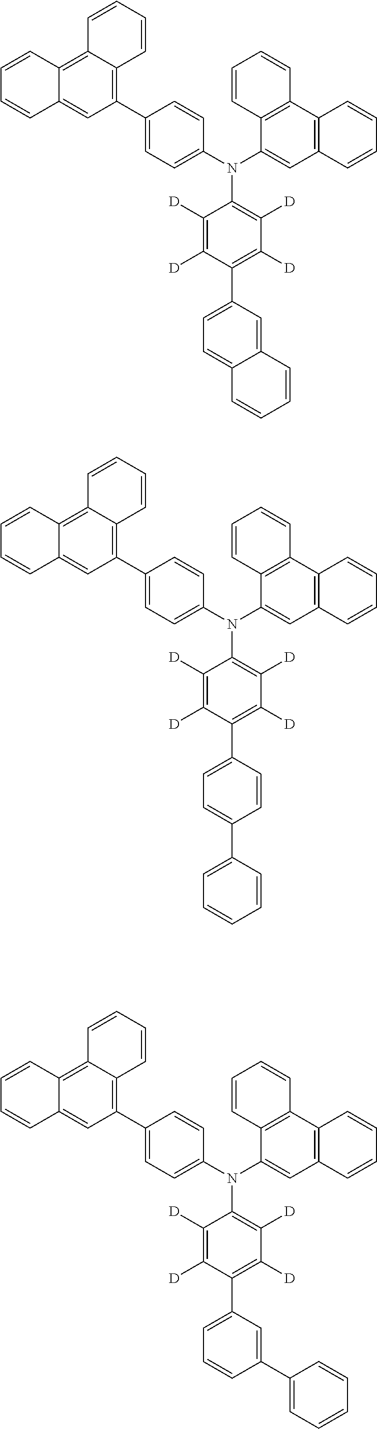 [SMILES: [2H]c1c([2H])c(N(c2ccc(-c3cc4ccccc4c4ccccc34)cc2)c2cc3ccccc3c3ccccc23)c([2H])c([2H])c1-c1ccc2ccccc2c1 ; [2H]c1c([2H])c(N(c2ccc(-c3cc4ccccc4c4ccccc34)cc2)c2cc3ccccc3c3ccccc23)c([2H])c([2H])c1-c1ccc(-c2ccccc2)cc1 ; [2H]c1c([2H])c(N(c2ccc(-c3cc4ccccc4c4ccccc34)cc2)c2cc3ccccc3c3ccccc23)c([2H])c([2H])c1-c1cccc(-c2ccccc2)c1]

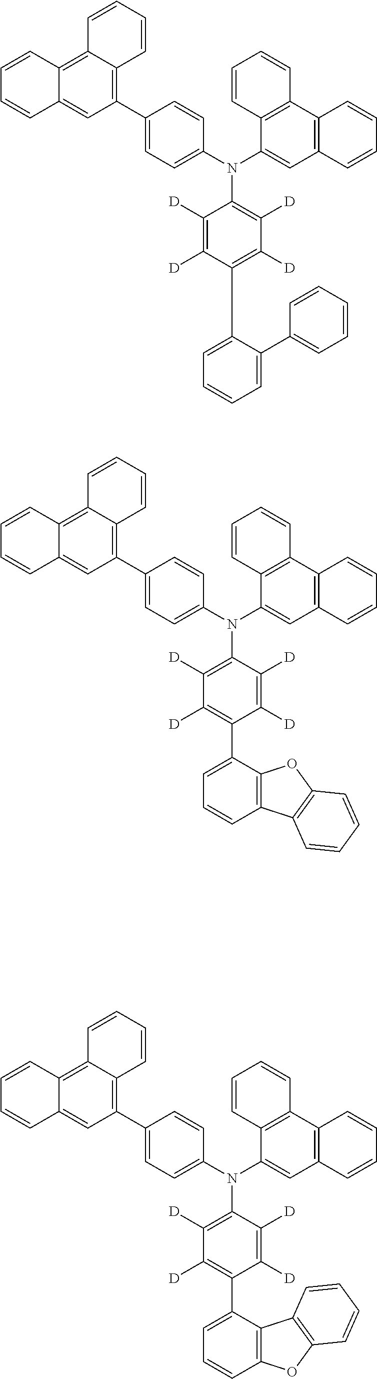 [SMILES: [2H]c1c([2H])c(N(c2ccc(-c3cc4ccccc4c4ccccc34)cc2)c2cc3ccccc3c3ccccc23)c([2H])c([2H])c1-c1ccccc1-c1ccccc1 ; [2H]c1c([2H])c(N(c2ccc(-c3cc4ccccc4c4ccccc34)cc2)c2cc3ccccc3c3ccccc23)c([2H])c([2H])c1-c1cccc2c1oc1ccccc12 ; [2H]c1c([2H])c(N(c2ccc(-c3cc4ccccc4c4ccccc34)cc2)c2cc3ccccc3c3ccccc23)c([2H])c([2H])c1-c1cccc2oc3ccccc3c12]

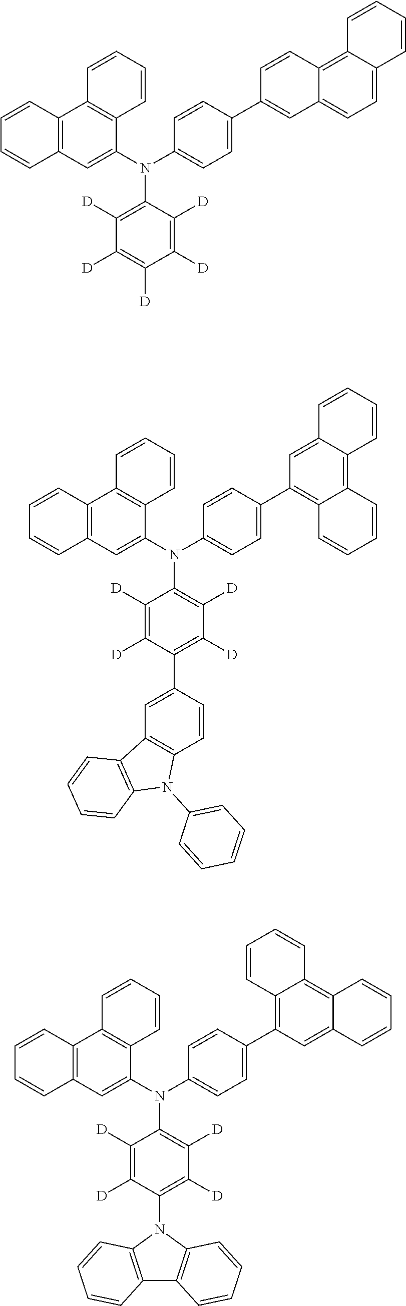 [SMILES: [2H]c1c([2H])c([2H])c(N(c2ccc(-c3ccc4c(ccc5ccccc54)c3)cc2)c2cc3ccccc3c3ccccc23)c([2H])c1[2H] ; [2H]c1c([2H])c(N(c2ccc(-c3cc4ccccc4c4ccccc34)cc2)c2cc3ccccc3c3ccccc23)c([2H])c([2H])c1-c1ccc2c(c1)c1ccccc1n2-c1ccccc1 ; [2H]c1c([2H])c(-n2c3ccccc3c3ccccc32)c([2H])c([2H])c1N(c1ccc(-c2cc3ccccc3c3ccccc23)cc1)c1cc2ccccc2c2ccccc12]

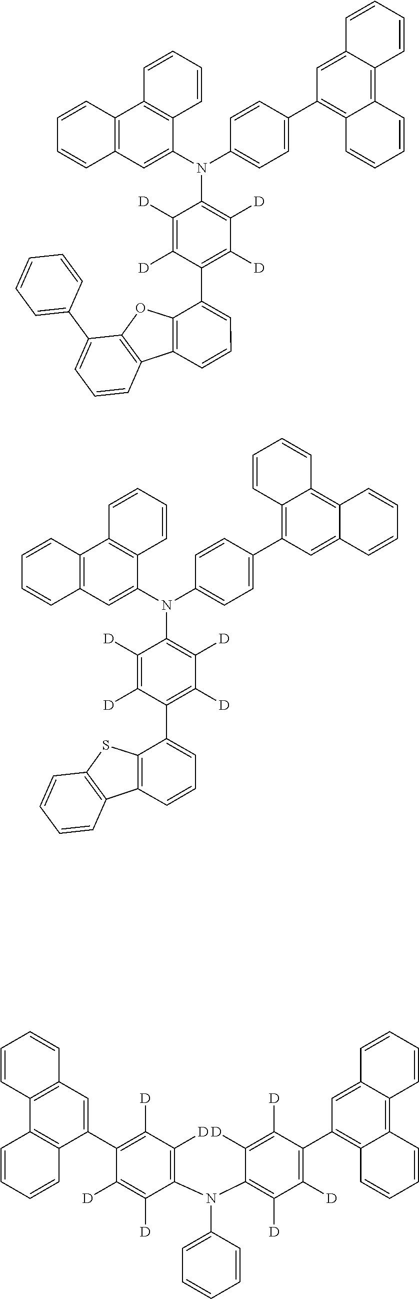 [SMILES: [2H]c1c([2H])c(N(c2ccc(-c3cc4ccccc4c4ccccc34)cc2)c2cc3ccccc3c3ccccc23)c([2H])c([2H])c1-c1cccc2c1oc1c(-c3ccccc3)cccc12 ; [2H]c1c([2H])c(N(c2ccc(-c3cc4ccccc4c4ccccc34)cc2)c2cc3ccccc3c3ccccc23)c([2H])c([2H])c1-c1cccc2c1sc1ccccc12 ; [2H]c1c([2H])c(N(c2ccccc2)c2c([2H])c([2H])c(-c3cc4ccccc4c4ccccc34)c([2H])c2[2H])c([2H])c([2H])c1-c1cc2ccccc2c2ccccc12]

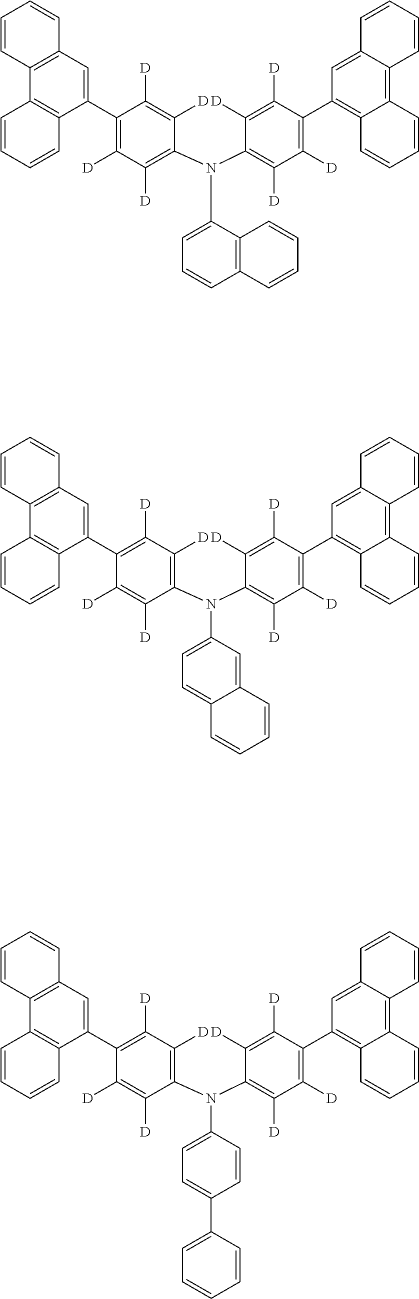 [SMILES: [2H]c1c([2H])c(N(c2c([2H])c([2H])c(-c3cc4ccccc4c4ccccc34)c([2H])c2[2H])c2cccc3ccccc23)c([2H])c([2H])c1-c1cc2ccccc2c2ccccc12 ; [2H]c1c([2H])c(N(c2ccc3ccccc3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccccc3)cc2)c2c([2H])c([2H])c(-c3cc4ccccc4c4ccccc34)c([2H])c2[2H])c([2H])c([2H])c1-c1cc2ccccc2c2ccccc12]

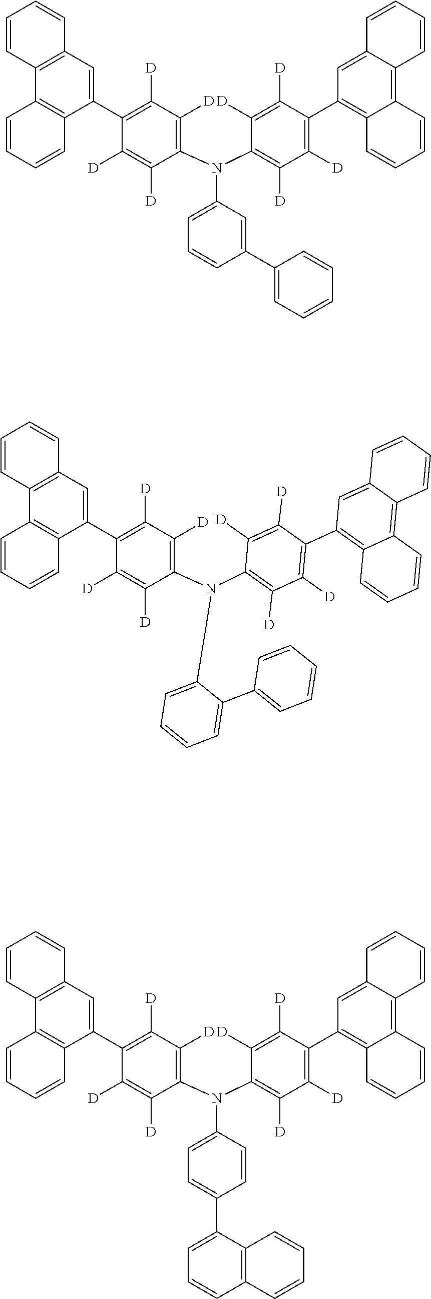 [SMILES: [2H]c1c([2H])c(N(c2cccc(-c3ccccc3)c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccccc2-c2ccccc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc4ccccc34)cc2)c2c([2H])c([2H])c(-c3cc4ccccc4c4ccccc34)c([2H])c2[2H])c([2H])c([2H])c1-c1cc2ccccc2c2ccccc12]

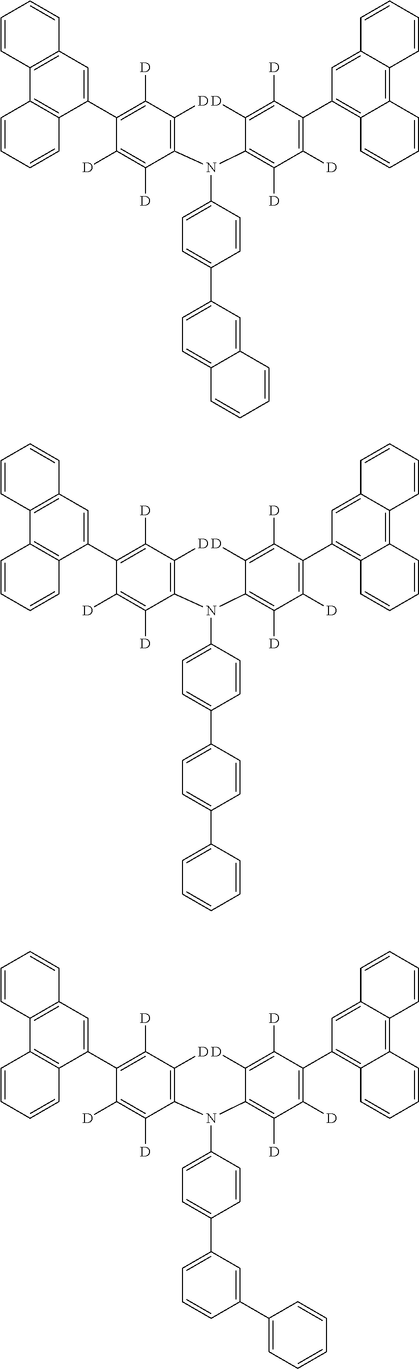 [SMILES: [2H]c1c([2H])c(N(c2ccc(-c3ccc4ccccc4c3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccc(-c4ccccc4)cc3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc(-c4ccccc4)c3)cc2)c2c([2H])c([2H])c(-c3cc4ccccc4c4ccccc34)c([2H])c2[2H])c([2H])c([2H])c1-c1cc2ccccc2c2ccccc12]

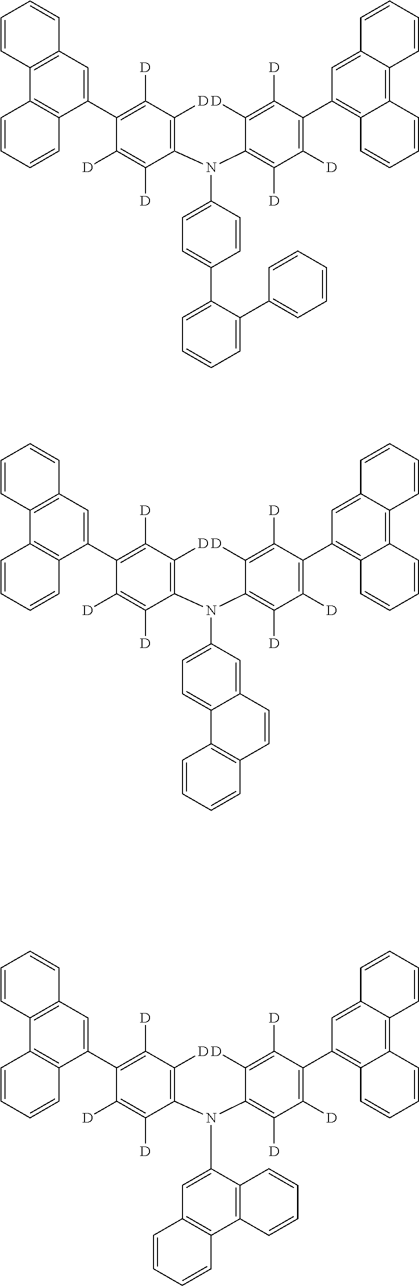 [SMILES: [2H]c1c([2H])c(N(c2ccc(-c3ccccc3-c3ccccc3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc3c(ccc4ccccc43)c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cc2ccccc2c2ccccc12]

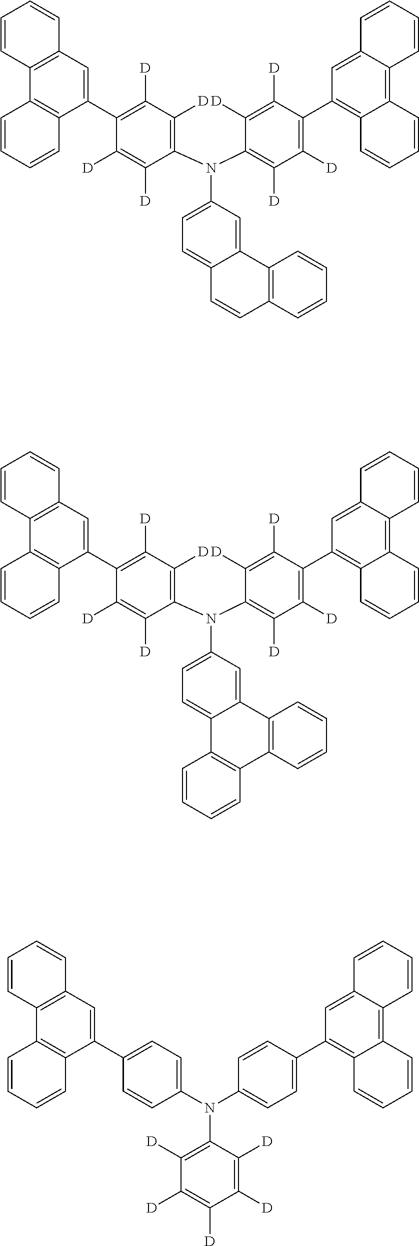 [SMILES: [2H]c1c([2H])c(N(c2ccc3ccc4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc3c4ccccc4c4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c([2H])c(N(c2ccc(-c3cc4ccccc4c4ccccc34)cc2)c2ccc(-c3cc4ccccc4c4ccccc34)cc2)c([2H])c1[2H]]

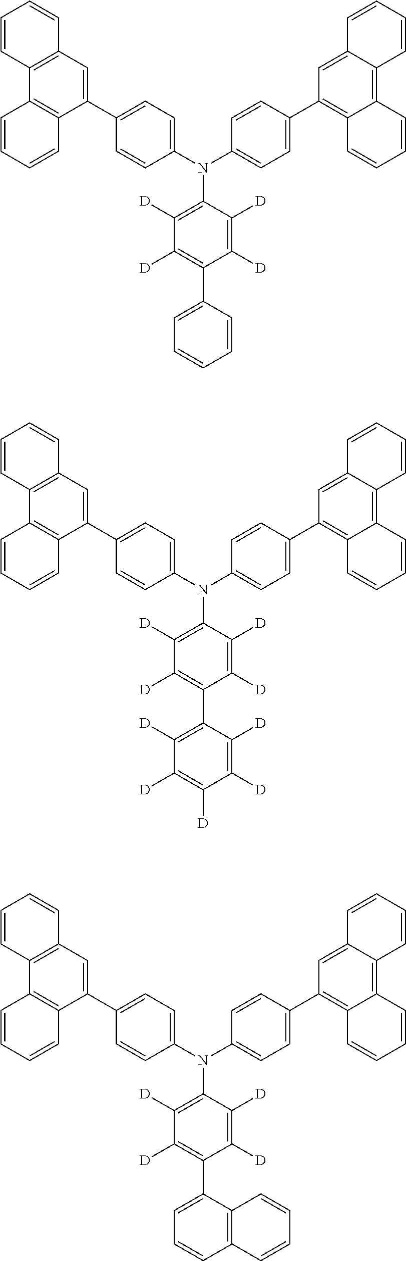 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1 ; [2H]c1c([2H])c([2H])c(-c2c([2H])c([2H])c(N(c3ccc(-c4cc5ccccc5c5ccccc45)cc3)c3ccc(-c4cc5ccccc5c5ccccc45)cc3)c([2H])c2[2H])c([2H])c1[2H] ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1cccc2ccccc12]

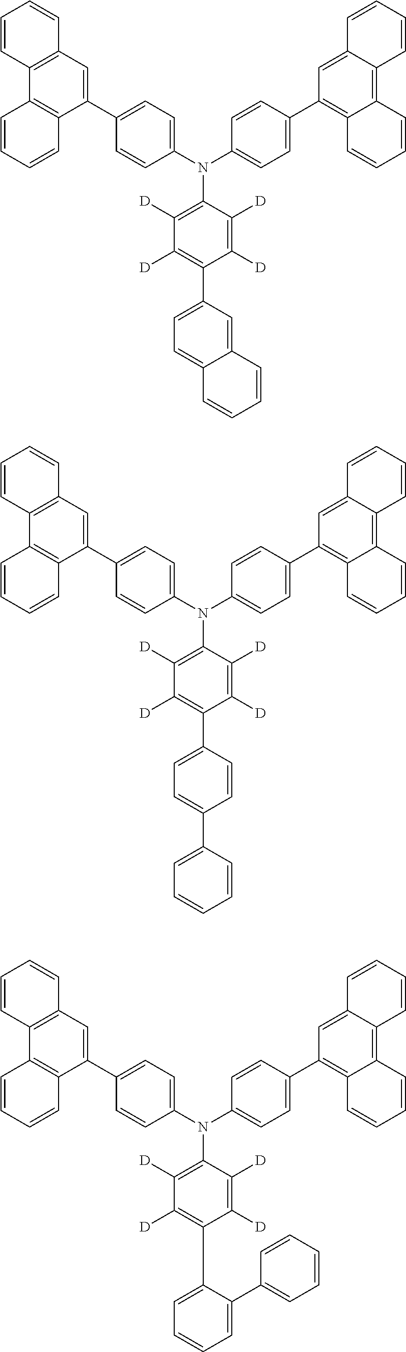 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc2ccccc2c1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc(-c2ccccc2)cc1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1-c1ccccc1]

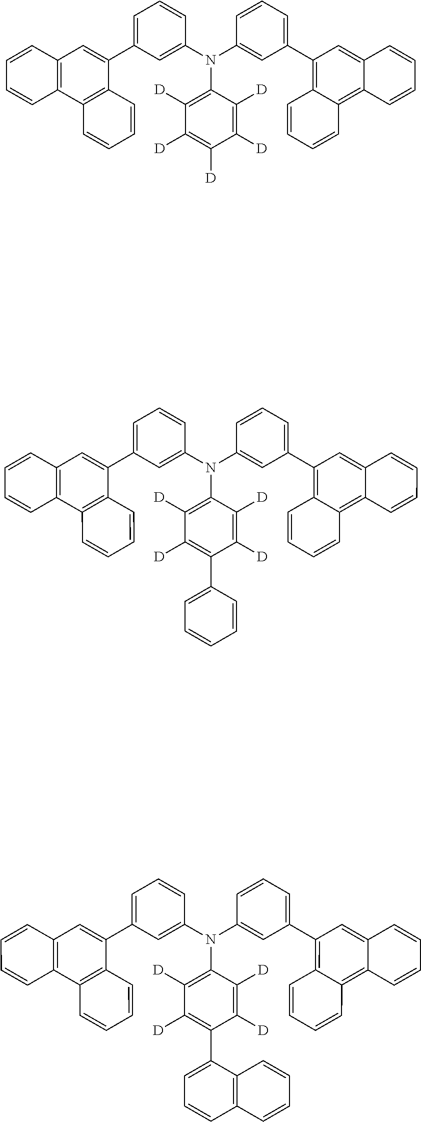 [SMILES: [2H]c1c([2H])c([2H])c(N(c2cccc(-c3cc4ccccc4c4ccccc34)c2)c2cccc(-c3cc4ccccc4c4ccccc34)c2)c([2H])c1[2H] ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccccc1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1cccc2ccccc12]

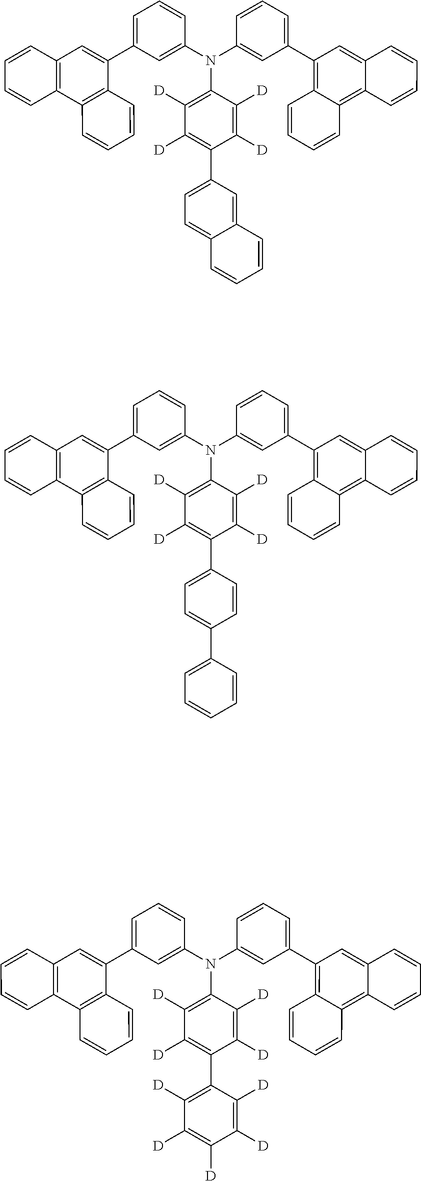 [SMILES: [2H]c1c([2H])c(N(c2cccc(-c3cc4ccccc4c4ccccc34)c2)c2cccc(-c3cc4ccccc4c4ccccc34)c2)c([2H])c([2H])c1-c1ccc2ccccc2c1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccc(-c2ccccc2)cc1 ; [2H]c1c([2H])c([2H])c(-c2c([2H])c([2H])c(N(c3cccc(-c4cc5ccccc5c5ccccc45)c3)c3cccc(-c4cc5ccccc5c5ccccc45)c3)c([2H])c2[2H])c([2H])c1[2H]]

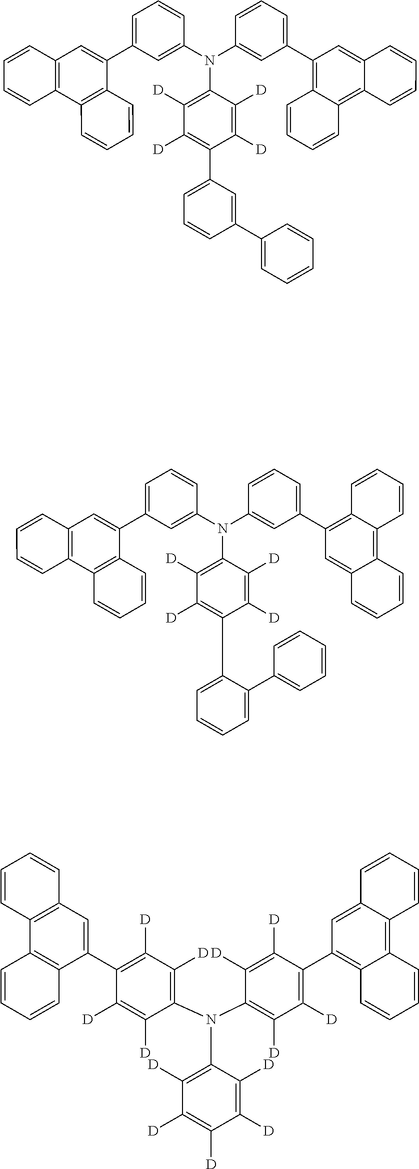 [SMILES: [2H]c1c([2H])c(N(c2cccc(-c3cc4ccccc4c4ccccc34)c2)c2cccc(-c3cc4ccccc4c4ccccc34)c2)c([2H])c([2H])c1-c1cccc(-c2ccccc2)c1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccccc1-c1ccccc1 ; [2H]c1c([2H])c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c1[2H]]

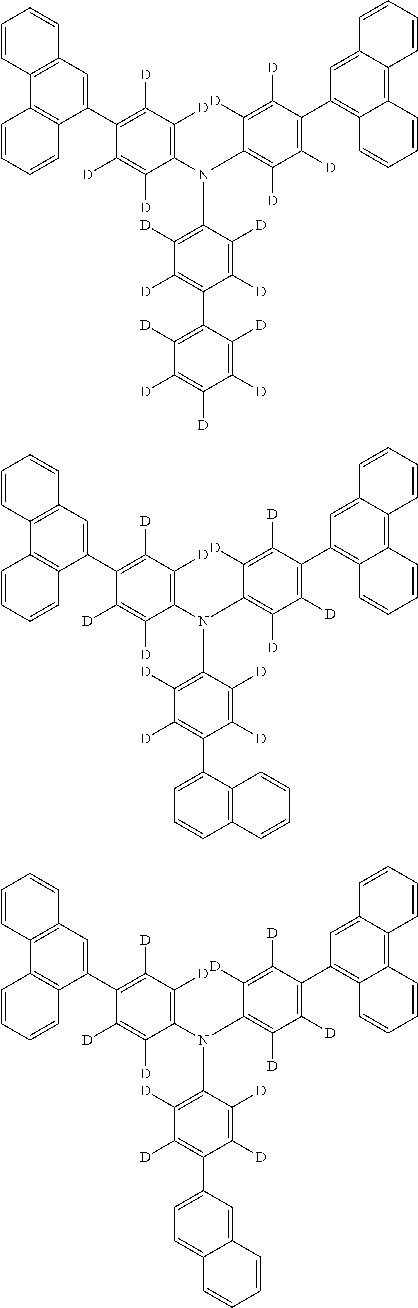 [SMILES: [2H]c1c([2H])c([2H])c(-c2c([2H])c([2H])c(N(c3c([2H])c([2H])c(-c4cc5ccccc5c5ccccc45)c([2H])c3[2H])c3c([2H])c([2H])c(-c4cc5ccccc5c5ccccc45)c([2H])c3[2H])c([2H])c2[2H])c([2H])c1[2H] ; [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1cccc2ccccc12 ; [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1ccc2ccccc2c1]

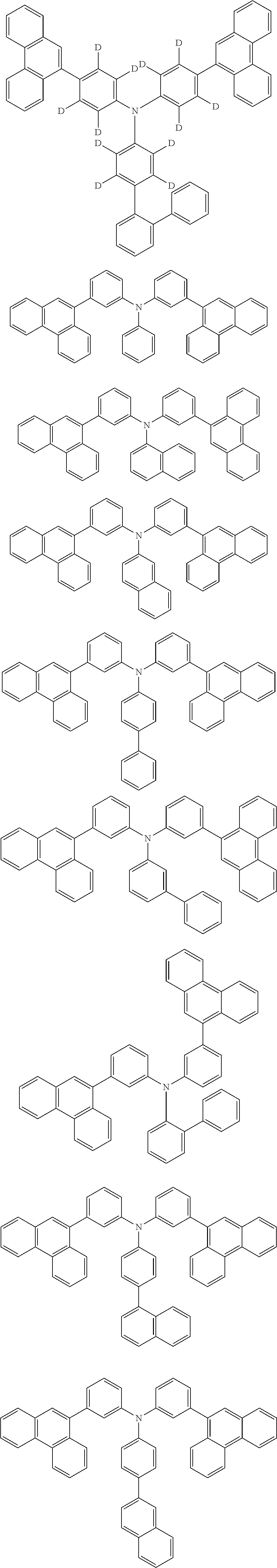

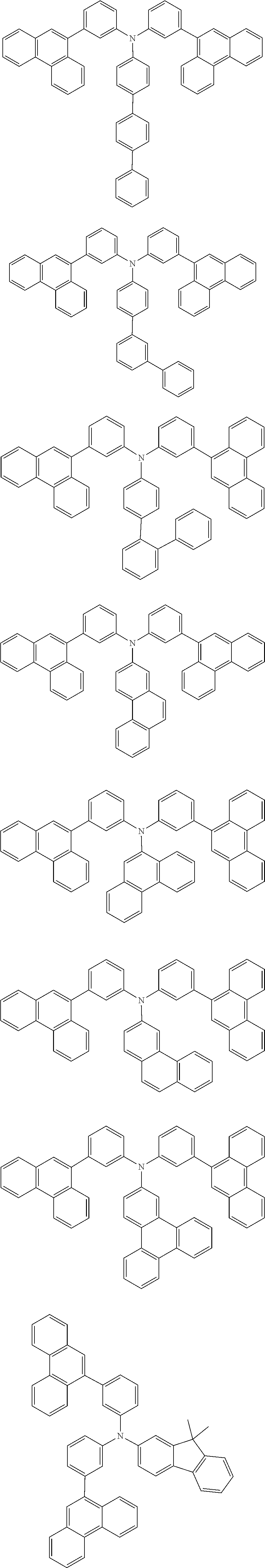

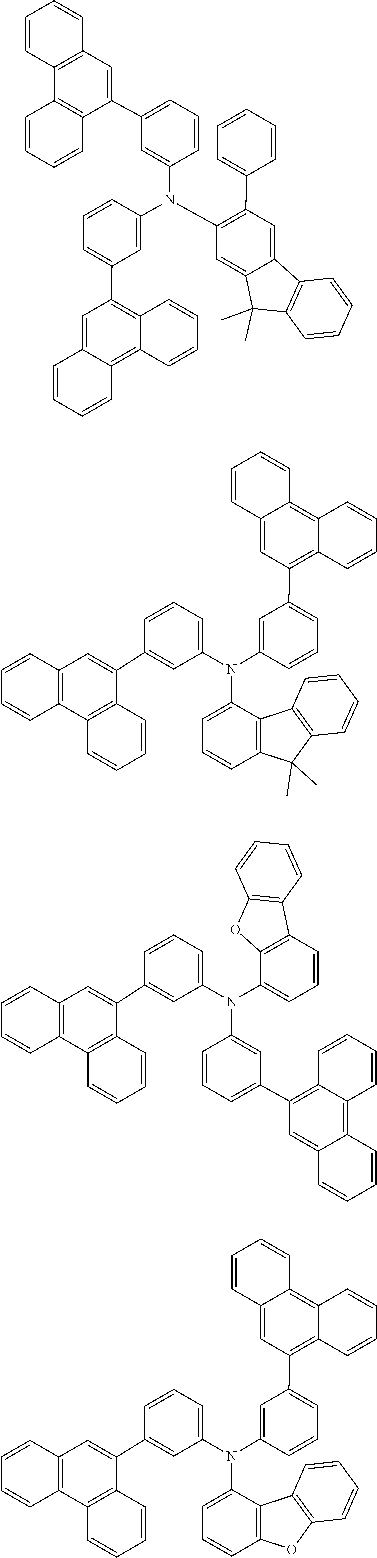 [SMILES: CC1(C)c2ccccc2-c2cc(-c3ccccc3)c(N(c3cccc(-c4cc5ccccc5c5ccccc45)c3)c3cccc(-c4cc5ccccc5c5ccccc45)c3)cc21 ; CC1(C)c2ccccc2-c2c(N(c3cccc(-c4cc5ccccc5c5ccccc45)c3)c3cccc(-c4cc5ccccc5c5ccccc45)c3)cccc21 ; c1cc(-c2cc3ccccc3c3ccccc23)cc(N(c2cccc(-c3cc4ccccc4c4ccccc34)c2)c2cccc3c2oc2ccccc23)c1 ; c1cc(-c2cc3ccccc3c3ccccc23)cc(N(c2cccc(-c3cc4ccccc4c4ccccc34)c2)c2cccc3oc4ccccc4c23)c1]

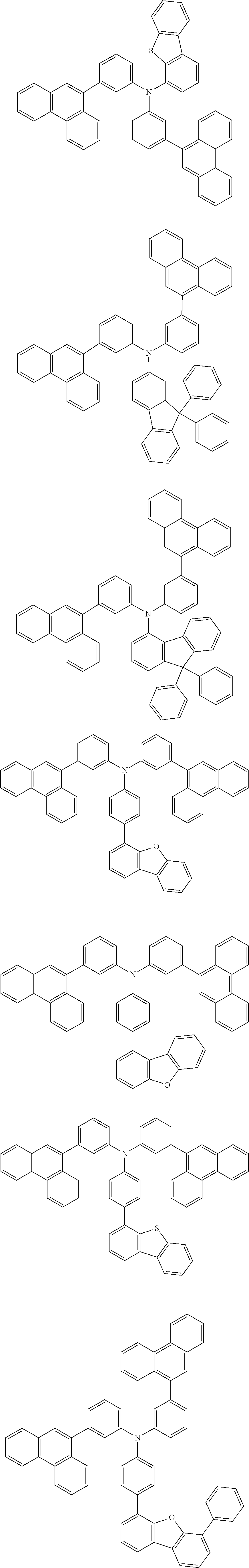

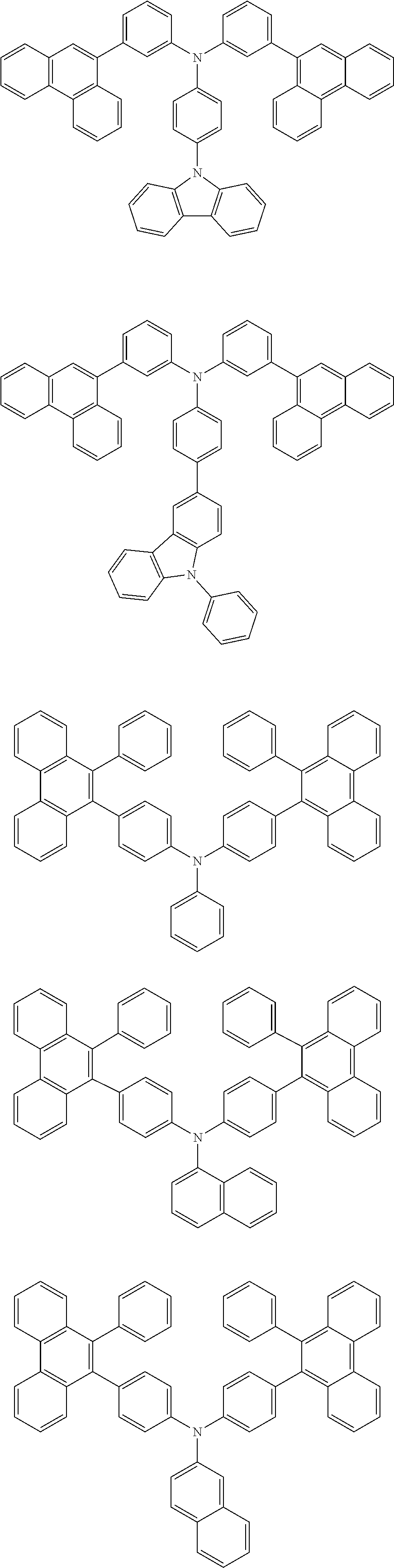

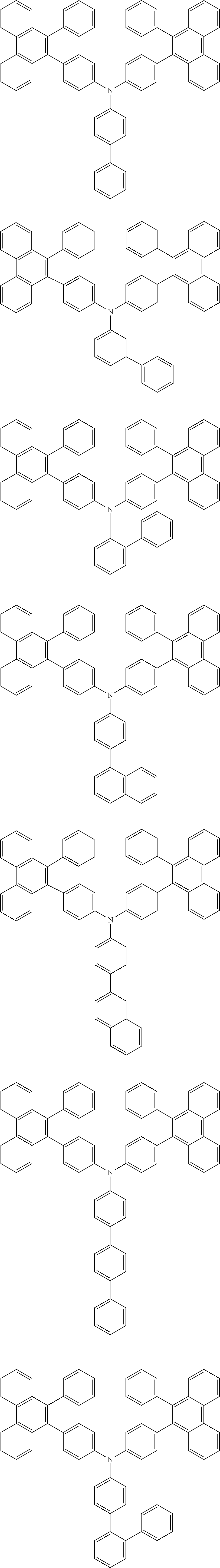

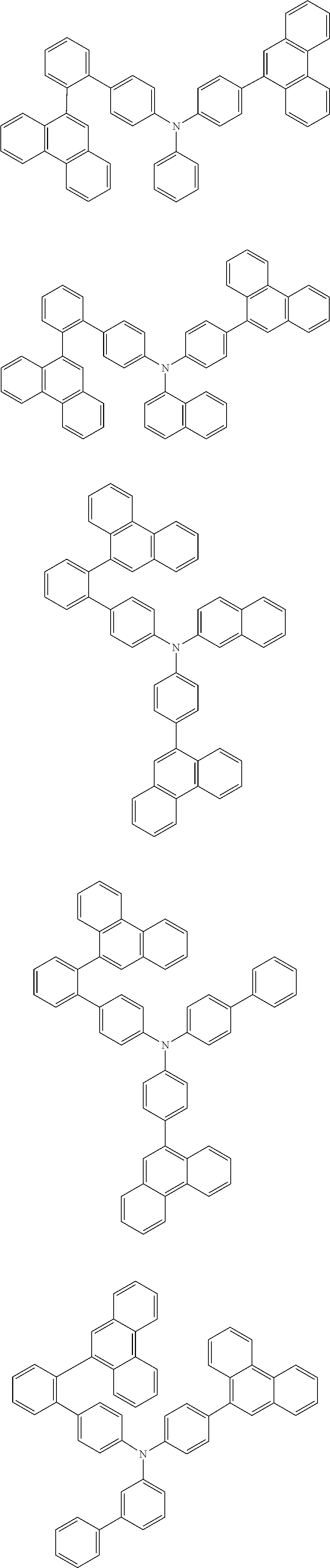 [SMILES: c1ccc(N(c2ccc(-c3ccccc3-c3cc4ccccc4c4ccccc34)cc2)c2ccc(-c3cc4ccccc4c4ccccc34)cc2)cc1 ; c1ccc(-c2cc3ccccc3c3ccccc23)c(-c2ccc(N(c3ccc(-c4cc5ccccc5c5ccccc45)cc3)c3cccc4ccccc34)cc2)c1 ; c1ccc(-c2cc3ccccc3c3ccccc23)c(-c2ccc(N(c3ccc(-c4cc5ccccc5c5ccccc45)cc3)c3ccc4ccccc4c3)cc2)c1 ; c1ccc(-c2ccc(N(c3ccc(-c4ccccc4-c4cc5ccccc5c5ccccc45)cc3)c3ccc(-c4cc5ccccc5c5ccccc45)cc3)cc2)cc1 ; c1ccc(-c2cccc(N(c3ccc(-c4ccccc4-c4cc5ccccc5c5ccccc45)cc3)c3ccc(-c4cc5ccccc5c5ccccc45)cc3)c2)cc1]

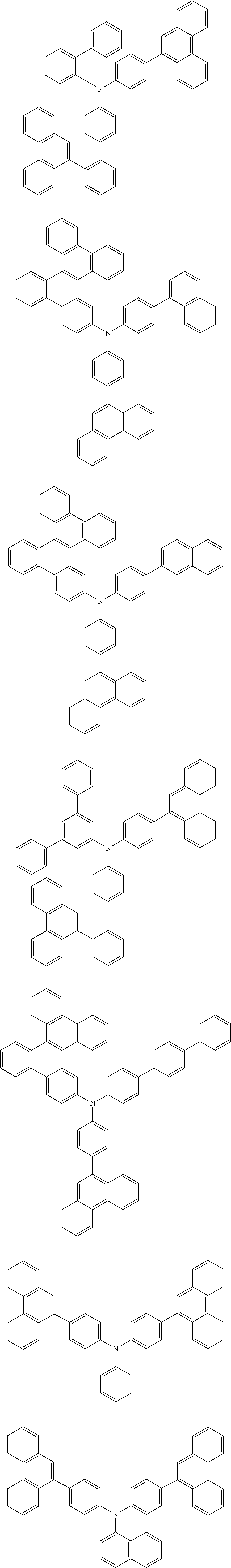

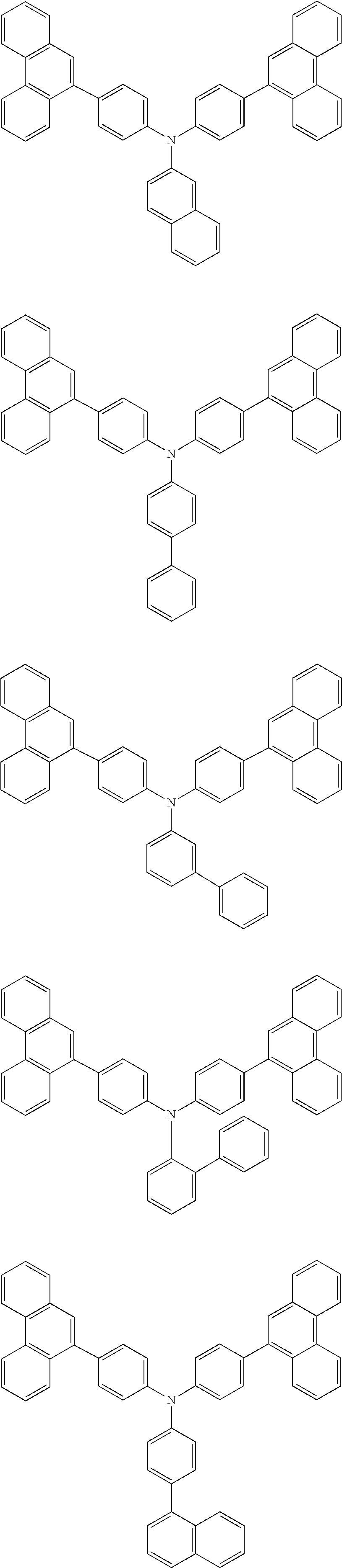 [SMILES: c1ccc2cc(N(c3ccc(-c4cc5ccccc5c5ccccc45)cc3)c3ccc(-c4cc5ccccc5c5ccccc45)cc3)ccc2c1 ; c1ccc(-c2ccc(N(c3ccc(-c4cc5ccccc5c5ccccc45)cc3)c3ccc(-c4cc5ccccc5c5ccccc45)cc3)cc2)cc1 ; c1ccc(-c2cccc(N(c3ccc(-c4cc5ccccc5c5ccccc45)cc3)c3ccc(-c4cc5ccccc5c5ccccc45)cc3)c2)cc1 ; c1ccc(-c2ccccc2N(c2ccc(-c3cc4ccccc4c4ccccc34)cc2)c2ccc(-c3cc4ccccc4c4ccccc34)cc2)cc1 ; c1ccc2c(-c3ccc(N(c4ccc(-c5cc6ccccc6c6ccccc56)cc4)c4ccc(-c5cc6ccccc6c6ccccc56)cc4)cc3)cccc2c1]

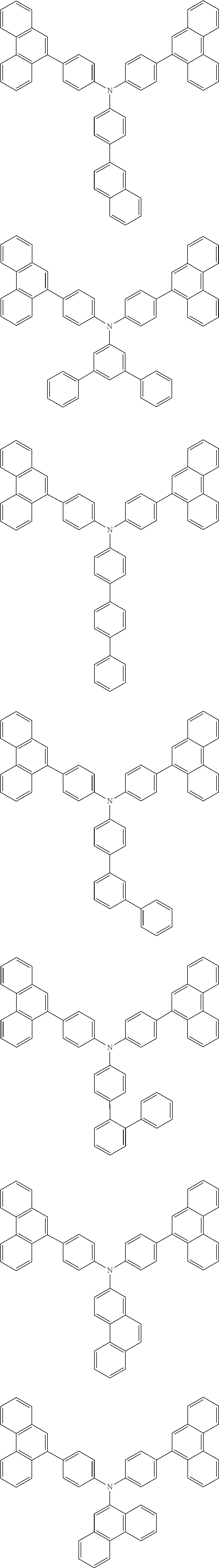

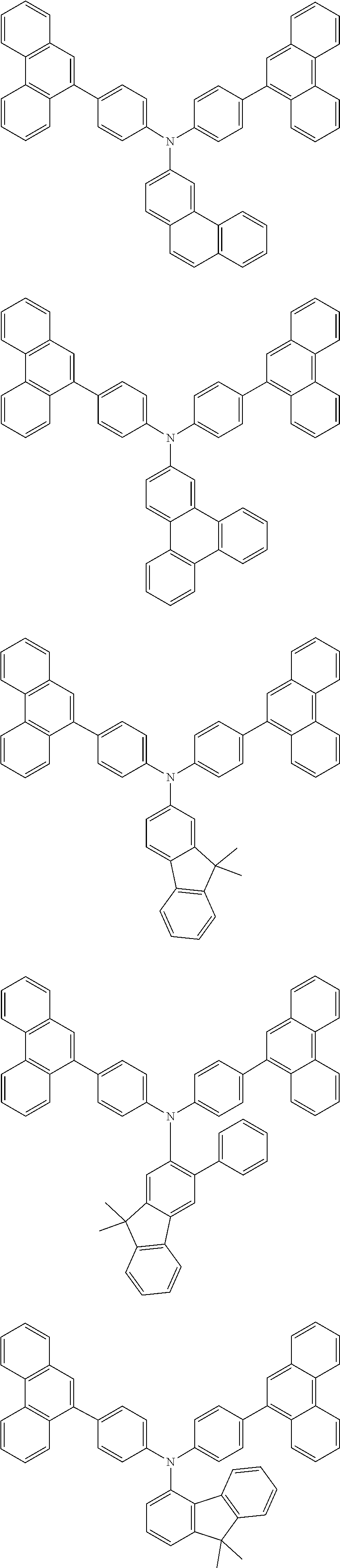 [SMILES: c1ccc2c(c1)ccc1ccc(N(c3ccc(-c4cc5ccccc5c5ccccc45)cc3)c3ccc(-c4cc5ccccc5c5ccccc45)cc3)cc12 ; c1ccc2c(c1)cc(-c1ccc(N(c3ccc(-c4cc5ccccc5c5ccccc45)cc3)c3ccc4c5ccccc5c5ccccc5c4c3)cc1)c1ccccc12 ; CC1(C)c2ccccc2-c2ccc(N(c3ccc(-c4cc5ccccc5c5ccccc45)cc3)c3ccc(-c4cc5ccccc5c5ccccc45)cc3)cc21 ; CC1(C)c2ccccc2-c2cc(-c3ccccc3)c(N(c3ccc(-c4cc5ccccc5c5ccccc45)cc3)c3ccc(-c4cc5ccccc5c5ccccc45)cc3)cc21 ; CC1(C)c2ccccc2-c2c(N(c3ccc(-c4cc5ccccc5c5ccccc45)cc3)c3ccc(-c4cc5ccccc5c5ccccc45)cc3)cccc21]

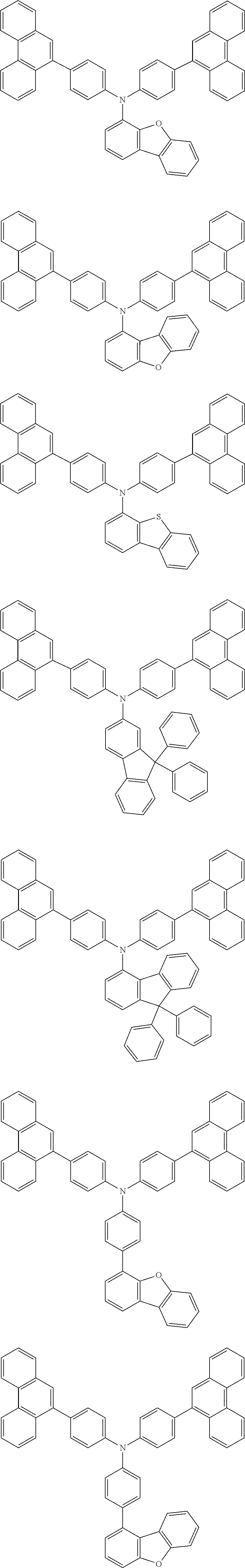

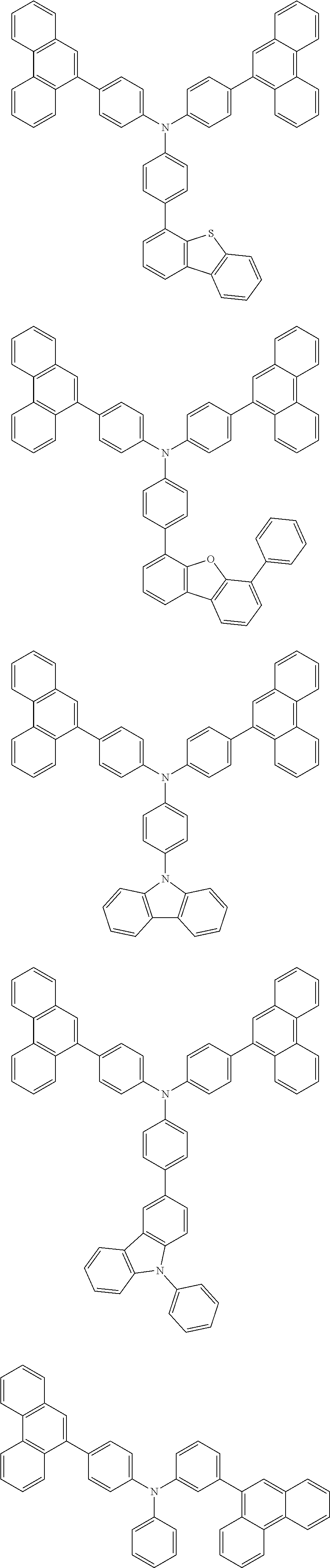

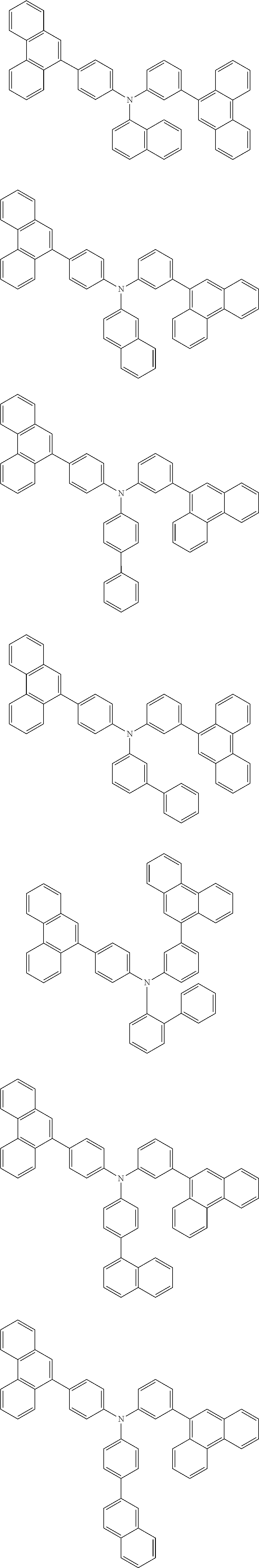

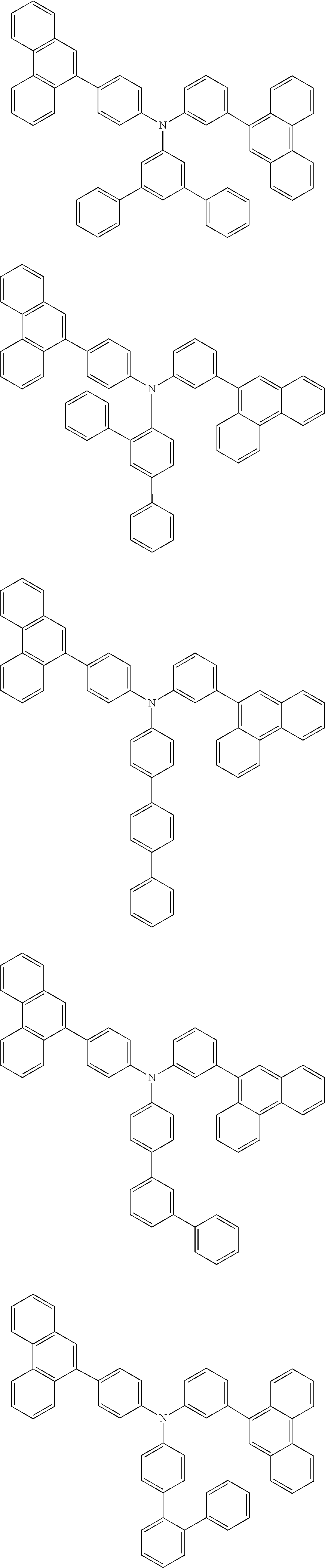 [SMILES: c1ccc(-c2cc(-c3ccccc3)cc(N(c3ccc(-c4cc5ccccc5c5ccccc45)cc3)c3cccc(-c4cc5ccccc5c5ccccc45)c3)c2)cc1 ; c1ccc(-c2ccc(N(c3ccc(-c4cc5ccccc5c5ccccc45)cc3)c3cccc(-c4cc5ccccc5c5ccccc45)c3)c(-c3ccccc3)c2)cc1 ; c1ccc(-c2ccc(-c3ccc(N(c4ccc(-c5cc6ccccc6c6ccccc56)cc4)c4cccc(-c5cc6ccccc6c6ccccc56)c4)cc3)cc2)cc1 ; c1ccc(-c2cccc(-c3ccc(N(c4ccc(-c5cc6ccccc6c6ccccc56)cc4)c4cccc(-c5cc6ccccc6c6ccccc56)c4)cc3)c2)cc1 ; c1ccc(-c2ccccc2-c2ccc(N(c3ccc(-c4cc5ccccc5c5ccccc45)cc3)c3cccc(-c4cc5ccccc5c5ccccc45)c3)cc2)cc1]

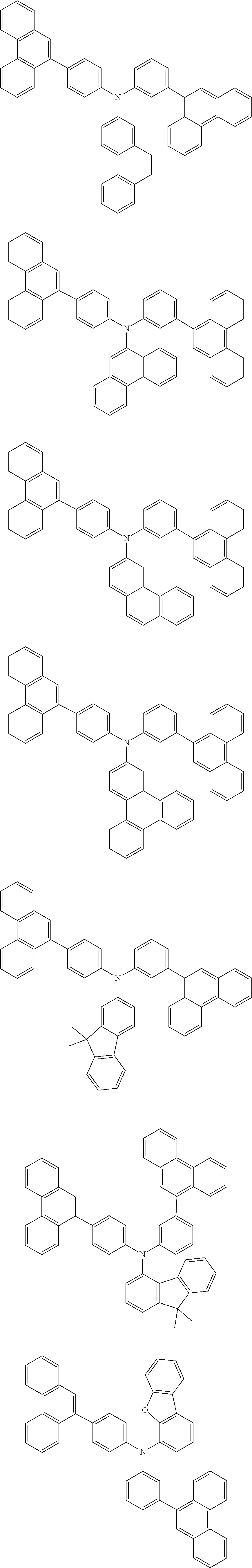

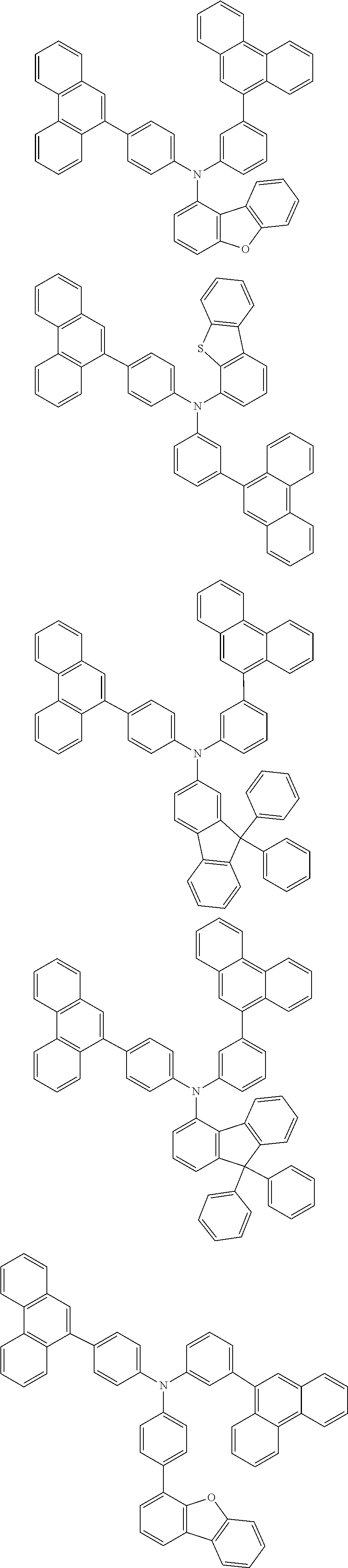

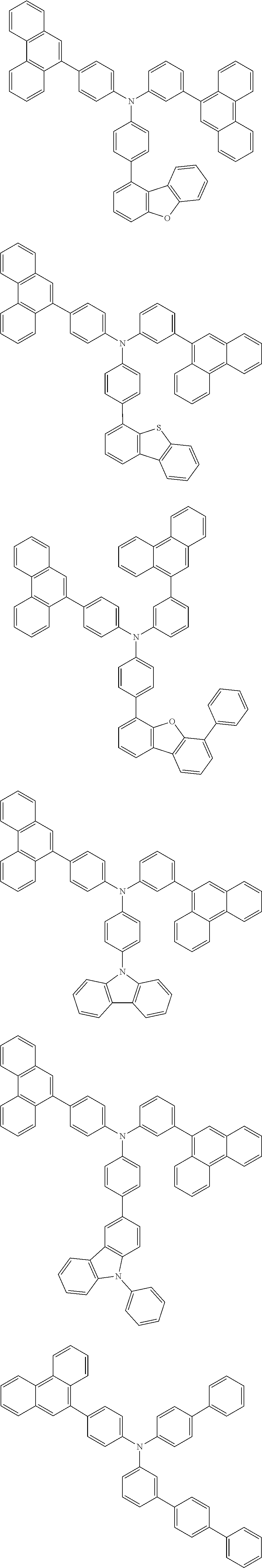

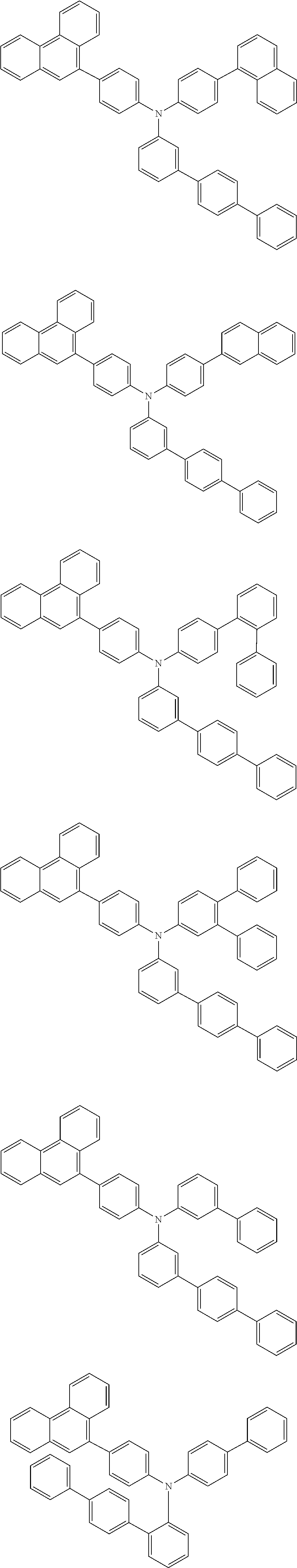

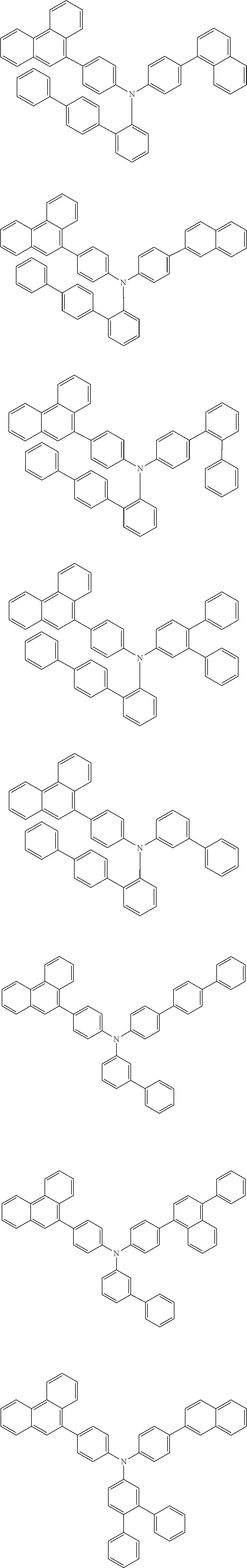

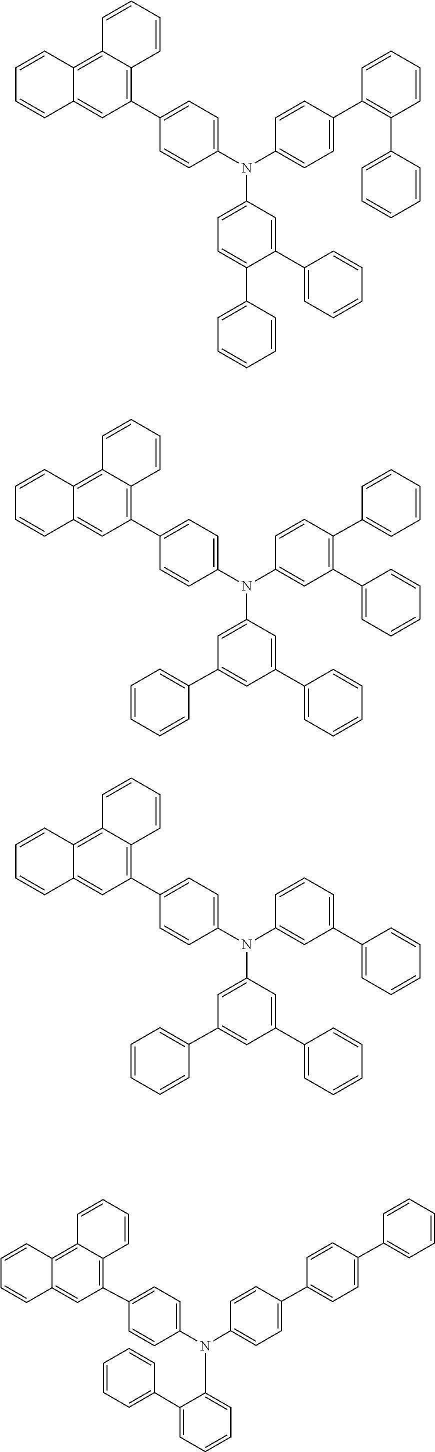 [SMILES: c1ccc(-c2ccccc2-c2ccc(N(c3ccc(-c4cc5ccccc5c5ccccc45)cc3)c3ccc(-c4ccccc4)c(-c4ccccc4)c3)cc2)cc1 ; c1ccc(-c2cc(-c3ccccc3)cc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cc(-c4ccccc4)cc(-c4ccccc4)c3)c2)cc1 ; c1ccc(-c2ccc(-c3ccc(N(c4ccc(-c5cc6ccccc6c6ccccc56)cc4)c4ccccc4-c4ccccc4)cc3)cc2)cc1]

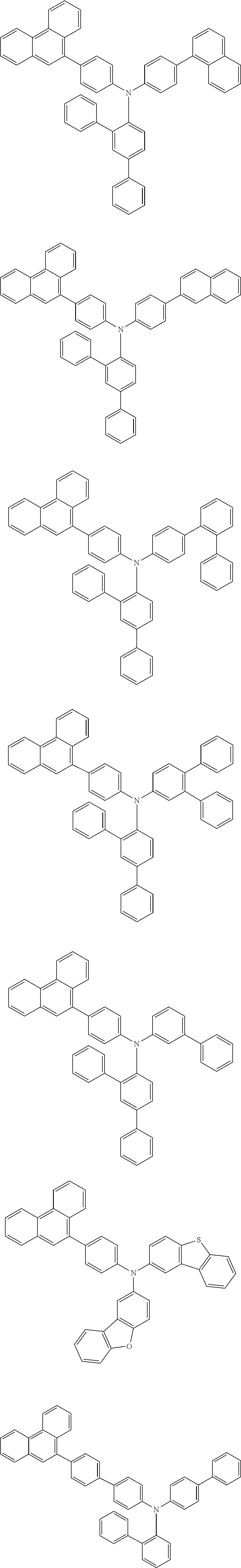

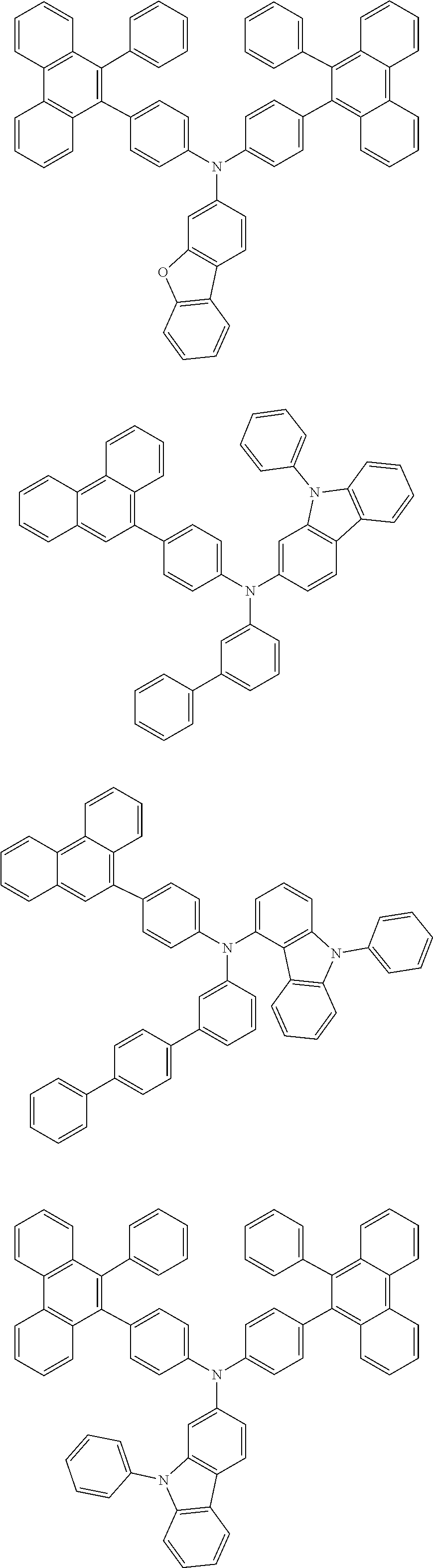 [SMILES: c1ccc(-c2c(-c3ccc(N(c4ccc(-c5c(-c6ccccc6)c6ccccc6c6ccccc56)cc4)c4ccc5c(c4)oc4ccccc45)cc3)c3ccccc3c3ccccc23)cc1 ; c1ccc(-c2cccc(N(c3ccc(-c4cc5ccccc5c5ccccc45)cc3)c3ccc4c5ccccc5n(-c5ccccc5)c4c3)c2)cc1 ; c1ccc(-c2ccc(-c3cccc(N(c4ccc(-c5cc6ccccc6c6ccccc56)cc4)c4cccc5c4c4ccccc4n5-c4ccccc4)c3)cc2)cc1 ; c1ccc(-c2c(-c3ccc(N(c4ccc(-c5c(-c6ccccc6)c6ccccc6c6ccccc56)cc4)c4ccc5c6ccccc6n(-c6ccccc6)c5c4)cc3)c3ccccc3c3ccccc23)cc1]

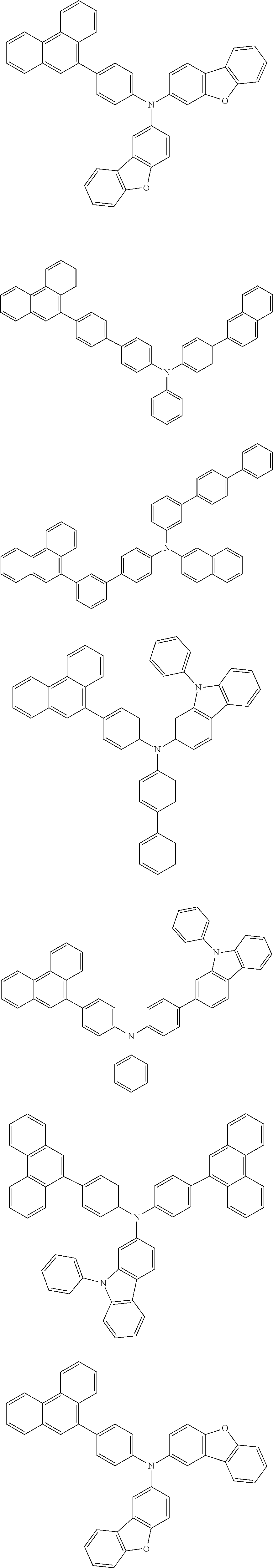

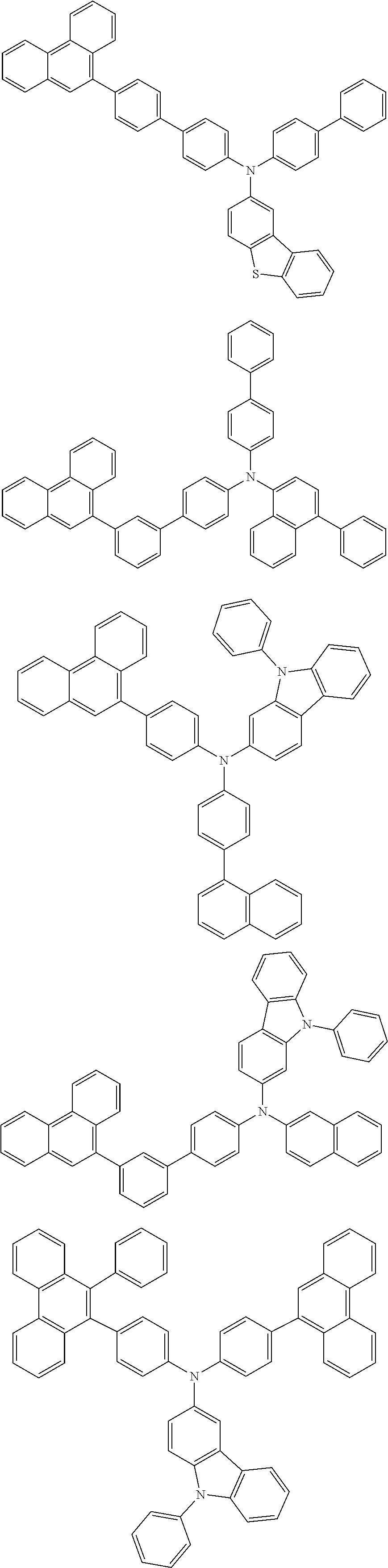

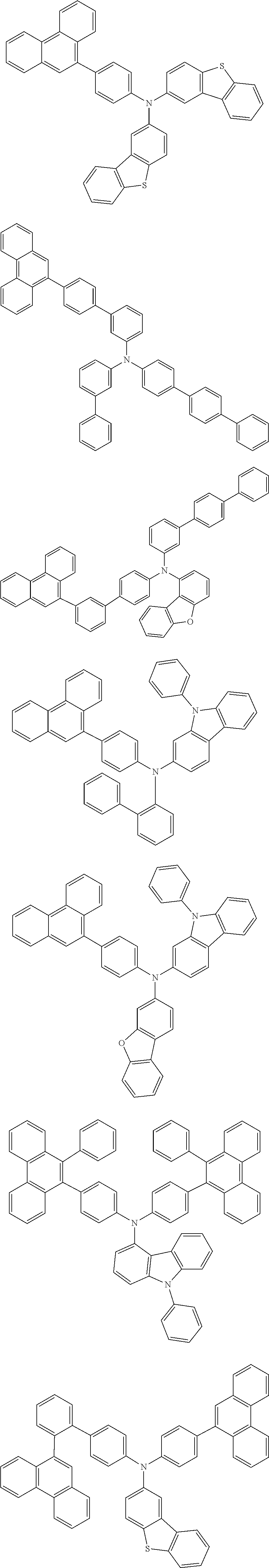

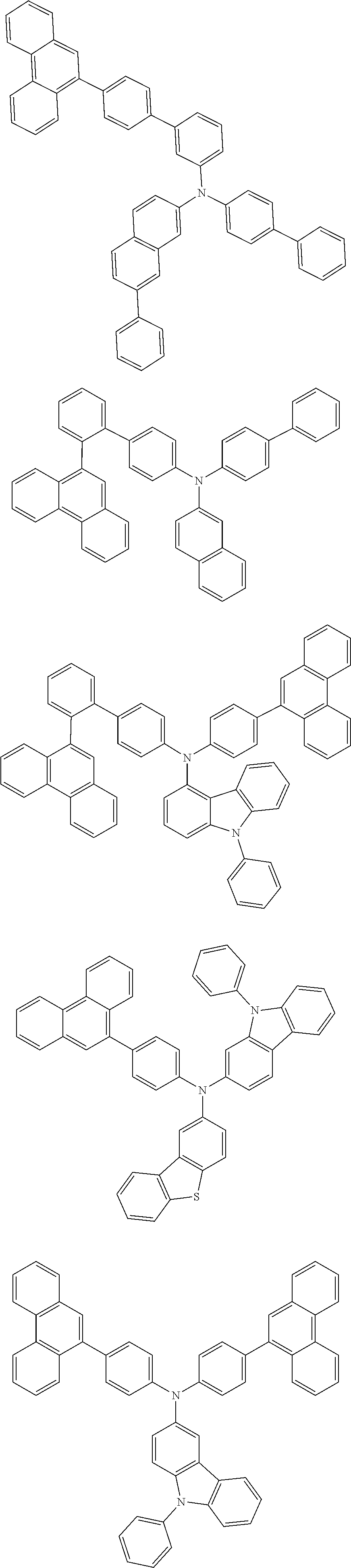 [SMILES: c1ccc(-c2ccc(N(c3cccc(-c4ccc(-c5cc6ccccc6c6ccccc56)cc4)c3)c3ccc4ccc(-c5ccccc5)cc4c3)cc2)cc1 ; c1ccc(-c2ccc(N(c3ccc(-c4ccccc4-c4cc5ccccc5c5ccccc45)cc3)c3ccc4ccccc4c3)cc2)cc1 ; c1ccc(-n2c3ccccc3c3c(N(c4ccc(-c5ccccc5-c5cc6ccccc6c6ccccc56)cc4)c4ccc(-c5cc6ccccc6c6ccccc56)cc4)cccc32)cc1 ; c1ccc(-n2c3ccccc3c3ccc(N(c4ccc(-c5cc6ccccc6c6ccccc56)cc4)c4ccc5sc6ccccc6c5c4)cc32)cc1 ; c1ccc(-n2c3ccccc3c3cc(N(c4ccc(-c5cc6ccccc6c6ccccc56)cc4)c4ccc(-c5cc6ccccc6c6ccccc56)cc4)ccc32)cc1]

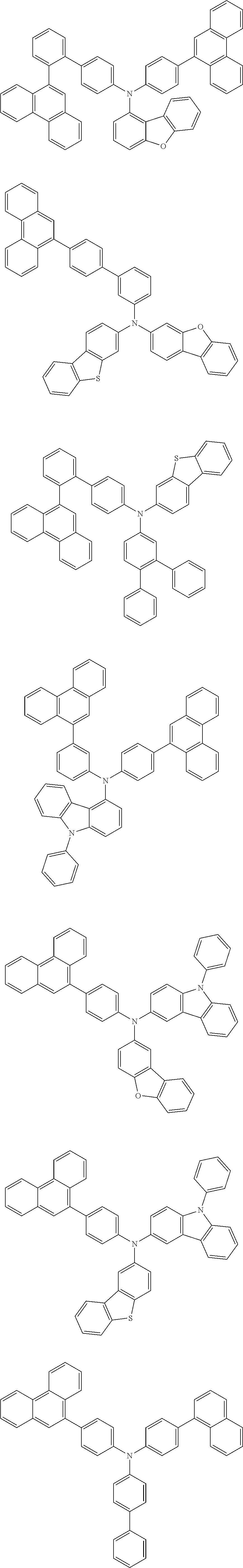

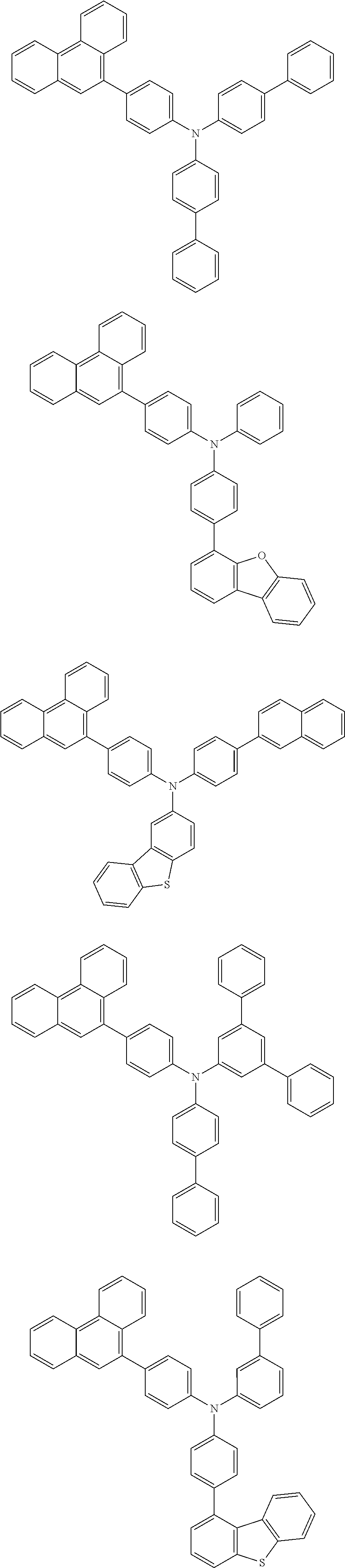 [SMILES: c1ccc(-c2ccc(N(c3ccc(-c4ccccc4)cc3)c3ccc(-c4cc5ccccc5c5ccccc45)cc3)cc2)cc1 ; c1ccc(N(c2ccc(-c3cc4ccccc4c4ccccc34)cc2)c2ccc(-c3cccc4c3oc3ccccc34)cc2)cc1 ; c1ccc2cc(-c3ccc(N(c4ccc(-c5cc6ccccc6c6ccccc56)cc4)c4ccc5sc6ccccc6c5c4)cc3)ccc2c1 ; c1ccc(-c2ccc(N(c3ccc(-c4cc5ccccc5c5ccccc45)cc3)c3cc(-c4ccccc4)cc(-c4ccccc4)c3)cc2)cc1 ; c1ccc(-c2cccc(N(c3ccc(-c4cc5ccccc5c5ccccc45)cc3)c3ccc(-c4cccc5sc6ccccc6c45)cc3)c2)cc1]

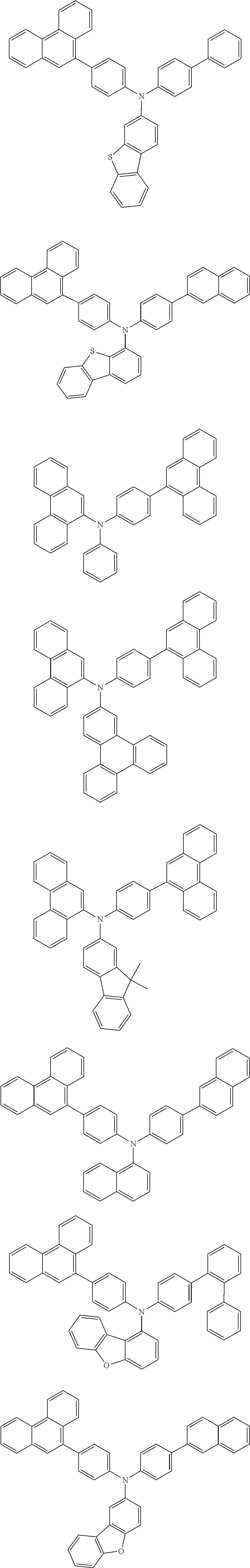

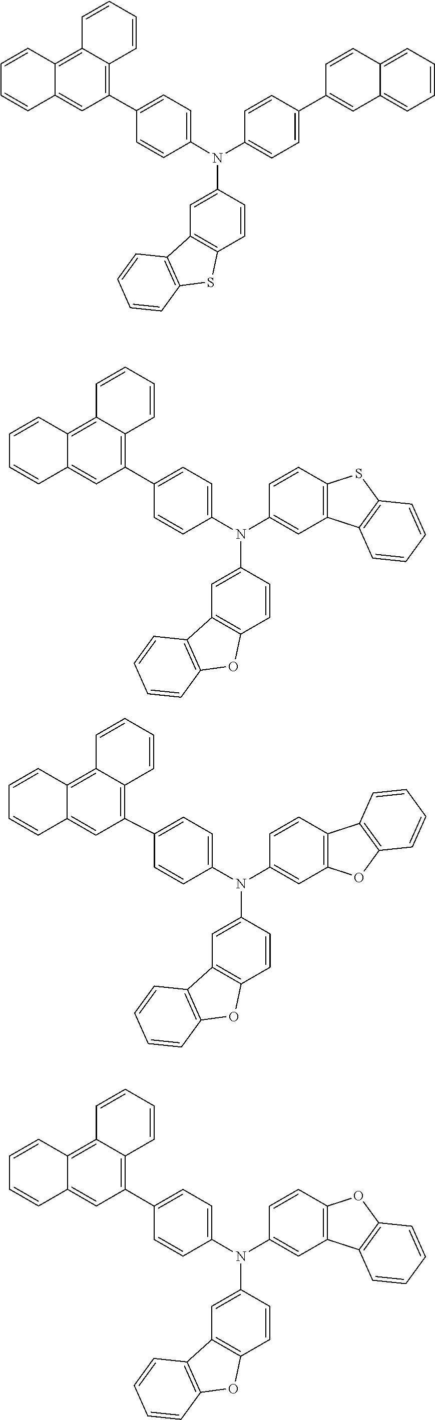

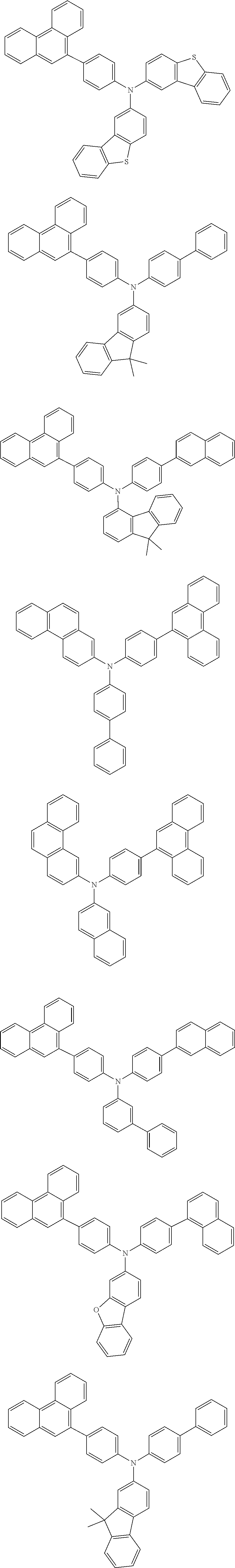

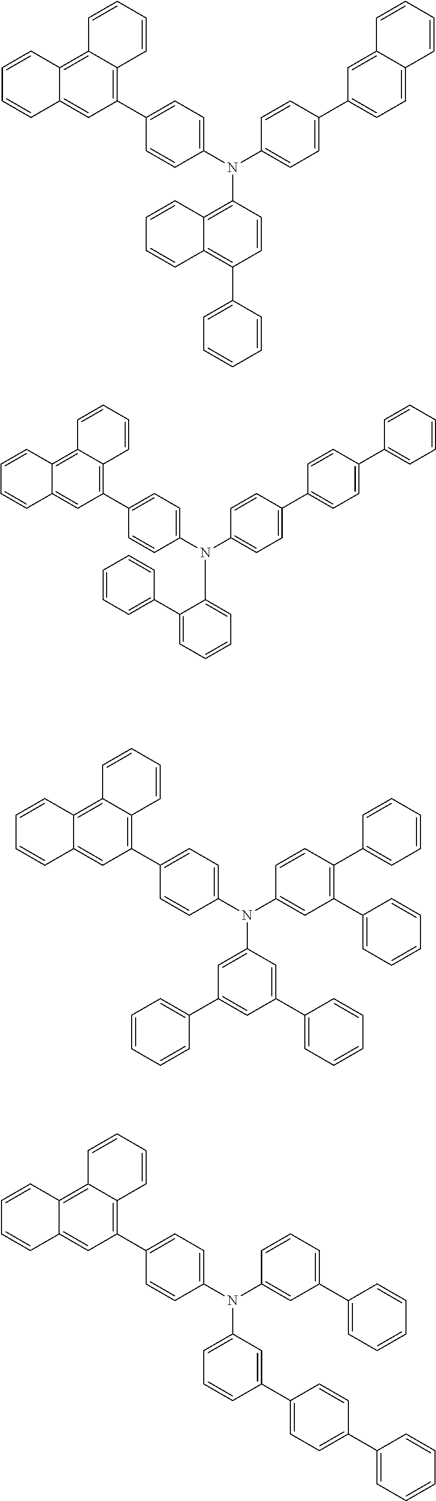 [SMILES: c1ccc(-c2ccc(N(c3ccc(-c4ccc5ccccc5c4)cc3)c3ccc(-c4cc5ccccc5c5ccccc45)cc3)c3ccccc23)cc1 ; c1ccc(-c2ccc(-c3ccc(N(c4ccc(-c5cc6ccccc6c6ccccc56)cc4)c4ccccc4-c4ccccc4)cc3)cc2)cc1 ; c1ccc(-c2cc(-c3ccccc3)cc(N(c3ccc(-c4cc5ccccc5c5ccccc45)cc3)c3ccc(-c4ccccc4)c(-c4ccccc4)c3)c2)cc1 ; c1ccc(-c2ccc(-c3cccc(N(c4ccc(-c5cc6ccccc6c6ccccc56)cc4)c4cccc(-c5ccccc5)c4)c3)cc2)cc1]

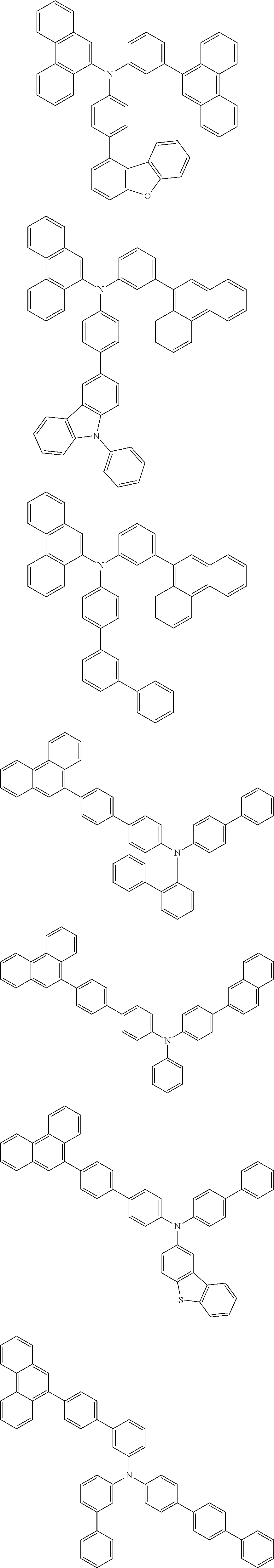

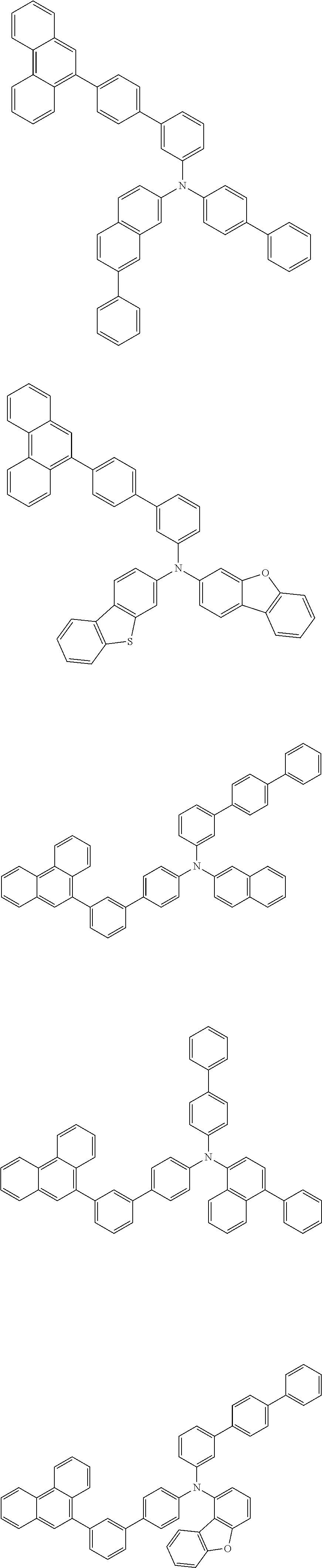 [SMILES: c1ccc(-c2ccc(N(c3cccc(-c4ccc(-c5cc6ccccc6c6ccccc56)cc4)c3)c3ccc4ccc(-c5ccccc5)cc4c3)cc2)cc1 ; c1cc(-c2ccc(-c3cc4ccccc4c4ccccc34)cc2)cc(N(c2ccc3c(c2)oc2ccccc23)c2ccc3c(c2)sc2ccccc23)c1 ; c1ccc(-c2ccc(-c3cccc(N(c4ccc(-c5cccc(-c6cc7ccccc7c7ccccc67)c5)cc4)c4ccc5ccccc5c4)c3)cc2)cc1 ; c1ccc(-c2ccc(N(c3ccc(-c4cccc(-c5cc6ccccc6c6ccccc56)c4)cc3)c3ccc(-c4ccccc4)c4ccccc34)cc2)cc1 ; c1ccc(-c2ccc(-c3cccc(N(c4ccc(-c5cccc(-c6cc7ccccc7c7ccccc67)c5)cc4)c4cccc5oc6ccccc6c45)c3)cc2)cc1]

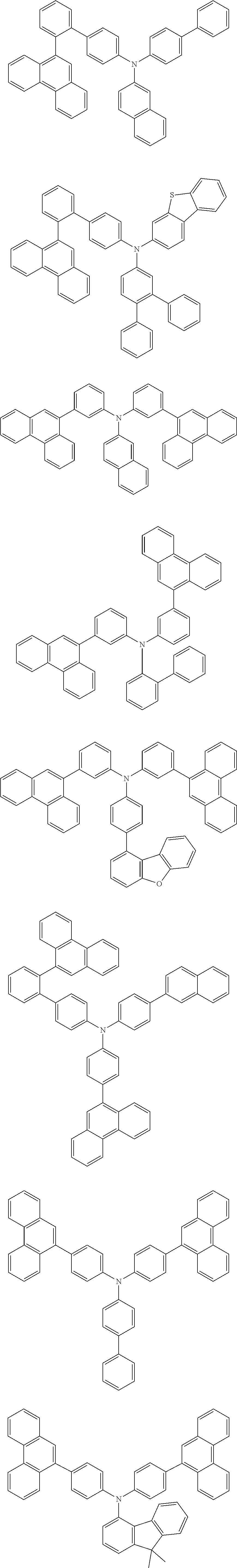

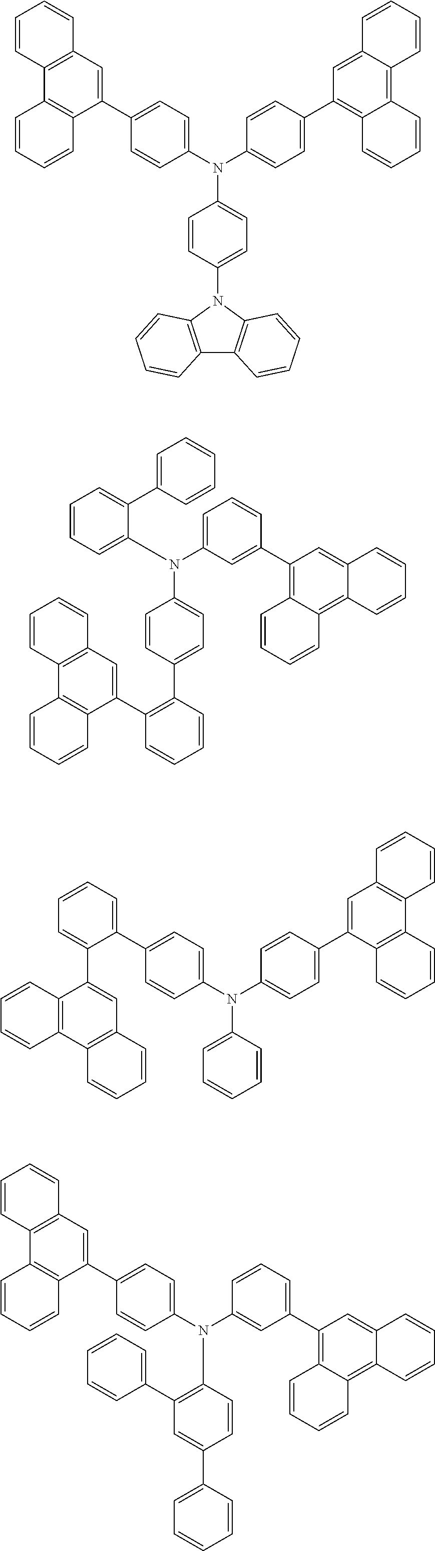 [SMILES: c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12 ; c1ccc(-c2ccccc2N(c2ccc(-c3ccccc3-c3cc4ccccc4c4ccccc34)cc2)c2cccc(-c3cc4ccccc4c4ccccc34)c2)cc1 ; c1ccc(N(c2ccc(-c3ccccc3-c3cc4ccccc4c4ccccc34)cc2)c2ccc(-c3cc4ccccc4c4ccccc34)cc2)cc1 ; c1ccc(-c2ccc(N(c3ccc(-c4cc5ccccc5c5ccccc45)cc3)c3cccc(-c4cc5ccccc5c5ccccc45)c3)c(-c3ccccc3)c2)cc1]

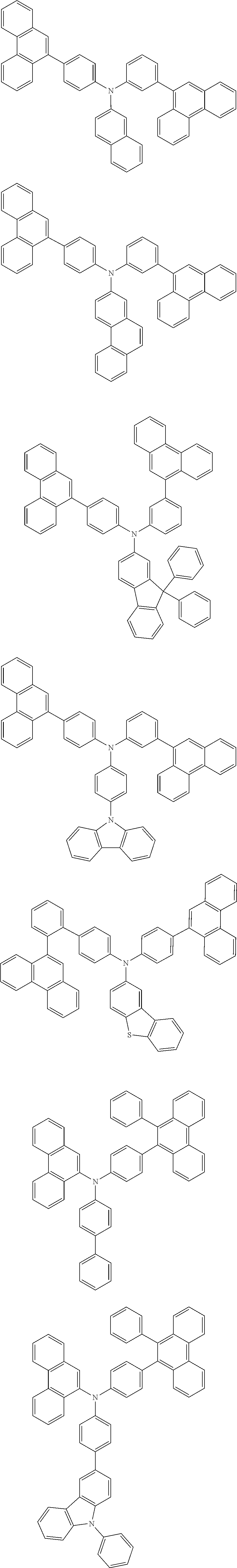

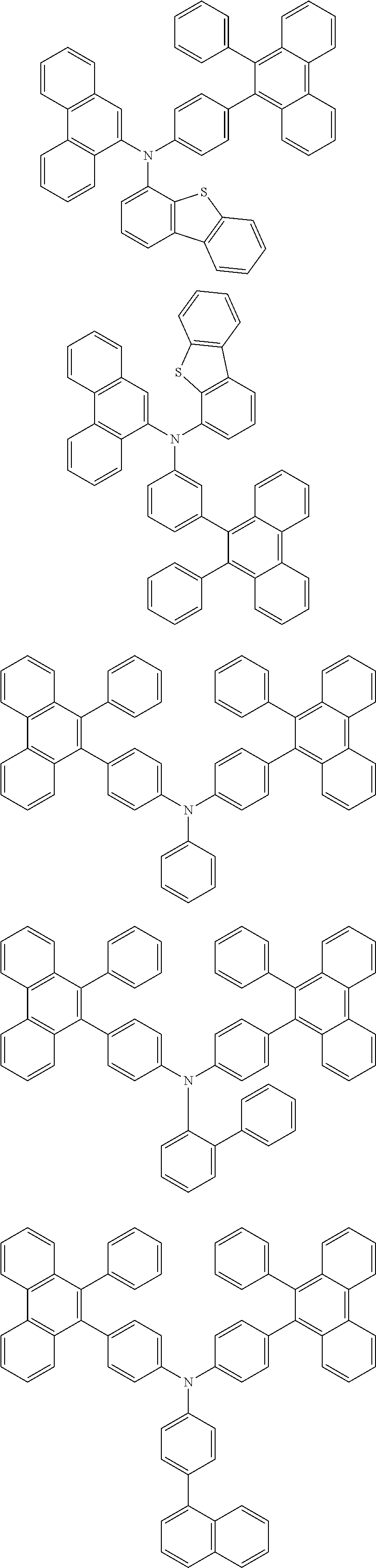

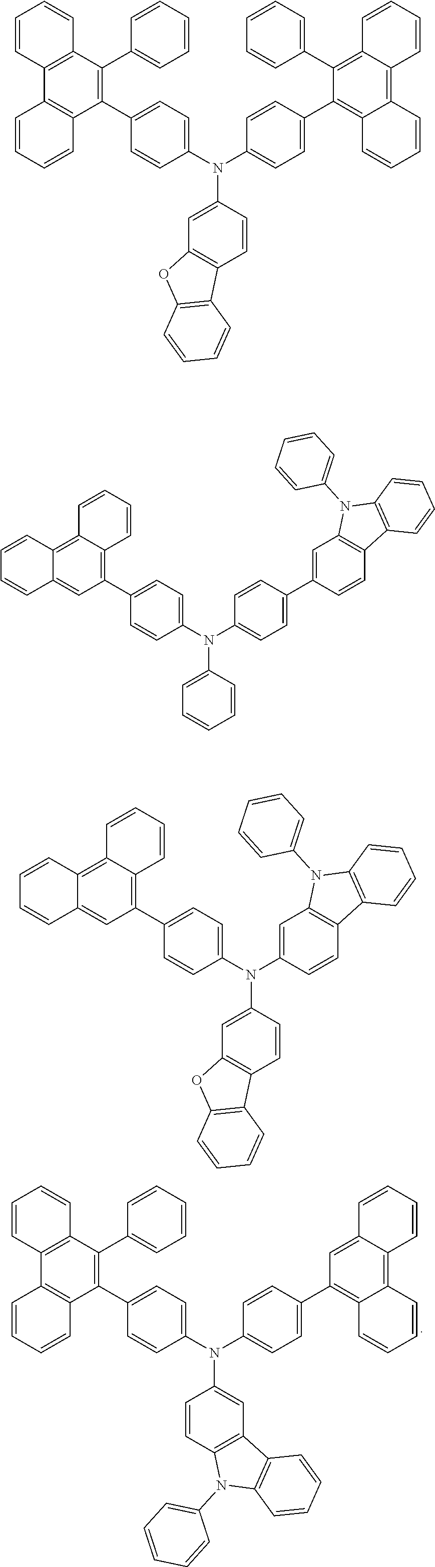 [SMILES: c1ccc(-c2c(-c3ccc(N(c4ccc(-c5c(-c6ccccc6)c6ccccc6c6ccccc56)cc4)c4ccc5c(c4)oc4ccccc45)cc3)c3ccccc3c3ccccc23)cc1 ; c1ccc(N(c2ccc(-c3ccc4c5ccccc5n(-c5ccccc5)c4c3)cc2)c2ccc(-c3cc4ccccc4c4ccccc34)cc2)cc1 ; c1ccc(-n2c3ccccc3c3ccc(N(c4ccc(-c5cc6ccccc6c6ccccc56)cc4)c4ccc5c(c4)oc4ccccc45)cc32)cc1 ; c1ccc(-c2c(-c3ccc(N(c4ccc(-c5cc6ccccc6c6ccccc56)cc4)c4ccc5c(c4)c4ccccc4n5-c4ccccc4)cc3)c3ccccc3c3ccccc23)cc1]

The compound of Chemical Formula 2 wherein A is

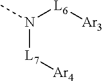

can be prepared by a preparation method as shown in the following Reaction Scheme 2 as an example, and the other remaining compounds can be prepared in a similar manner.

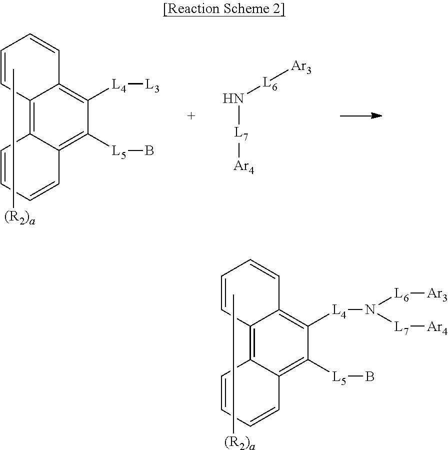

In Reaction Scheme 2, B, R₂, a, L₄ to L₇, Ar₃ and Ar₄ are the same as defined in Reaction Formula 2, and Z₃ is halogen, preferably Z₃ is chloro or bromo.

Reaction Scheme 2 is an amine substitution reaction, which is preferably carried out in the presence of a palladium catalyst and a base, and a reactive group for the amine substitution reaction can be modified as known in the art. The preparation method can be further embodied in Preparation Examples described hereinafter.

Preferably, the weight ratio of the compound of Chemical Formula 1 and the compound of Chemical Formula 2 in the light emitting layer can be 10:90 to 90:10, more preferably 20:80 to 80:20, 30:70 to 70:30 or 40:60 to 60:40.

Meanwhile, the light emitting layer can further include a dopant in addition to the host. The dopant material is not particularly limited as long as it is a material used for the organic light emitting device. As an example, an aromatic amine derivative, a styrylamine compound, a boron complex, a fluoranthene compound, a metal complex, and the like can be mentioned. Specific examples of the aromatic amine derivatives include substituted or unsubstituted fused aromatic ring derivatives having an arylamino group, examples thereof include pyrene, anthracene, chrysene, and periflanthene having the arylamino group, and the like. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, examples of the metal complex include an iridium complex, a platinum complex, and the like, but are not limited thereto. In one example, one or more selected from the following group can be used as a dopant material, but is not limited thereto:

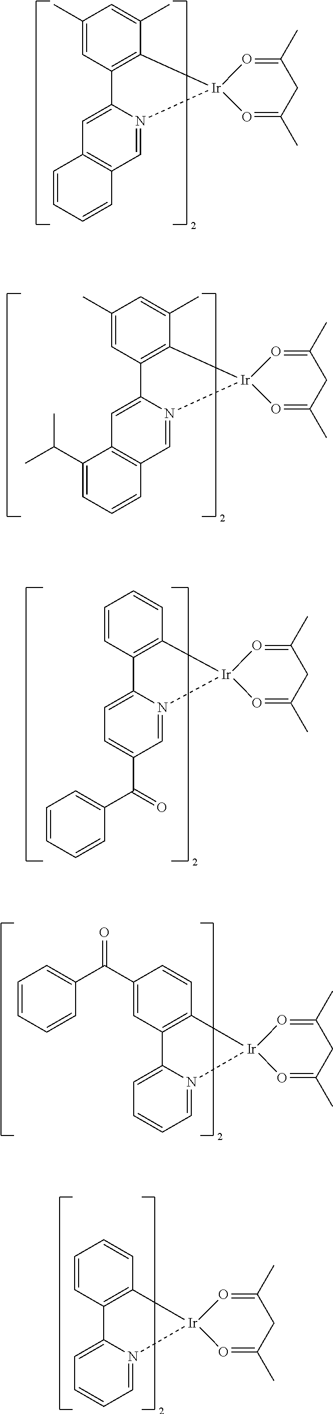

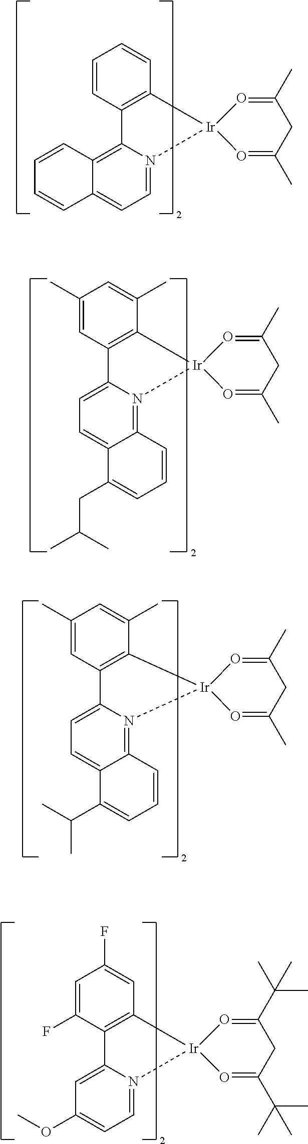

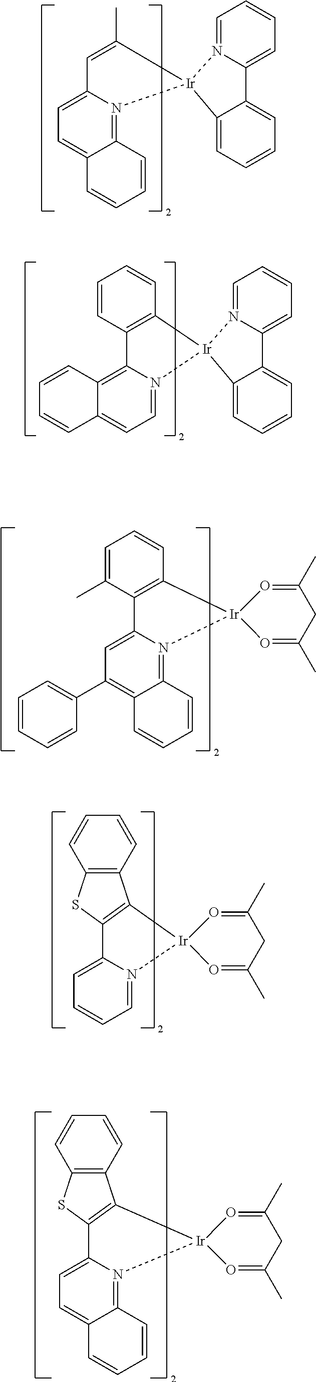

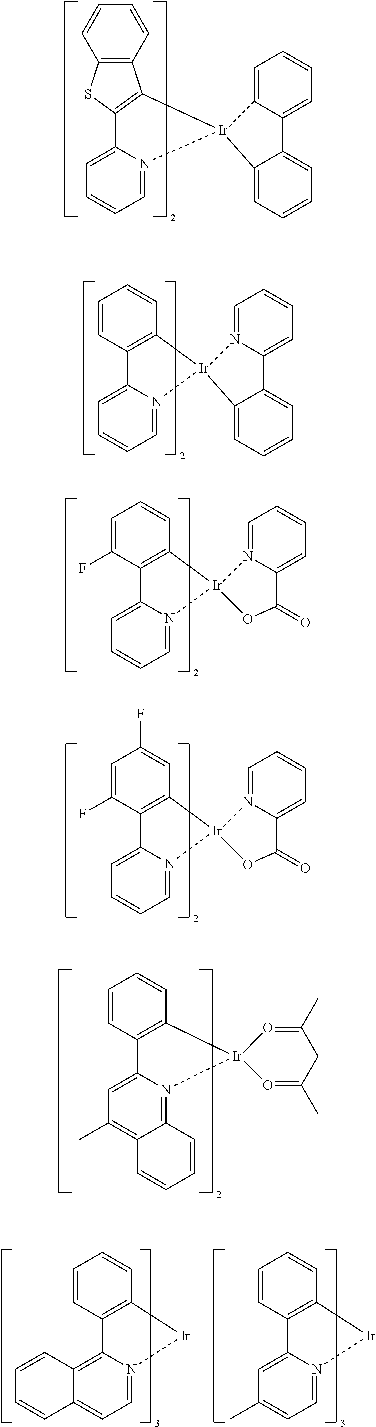

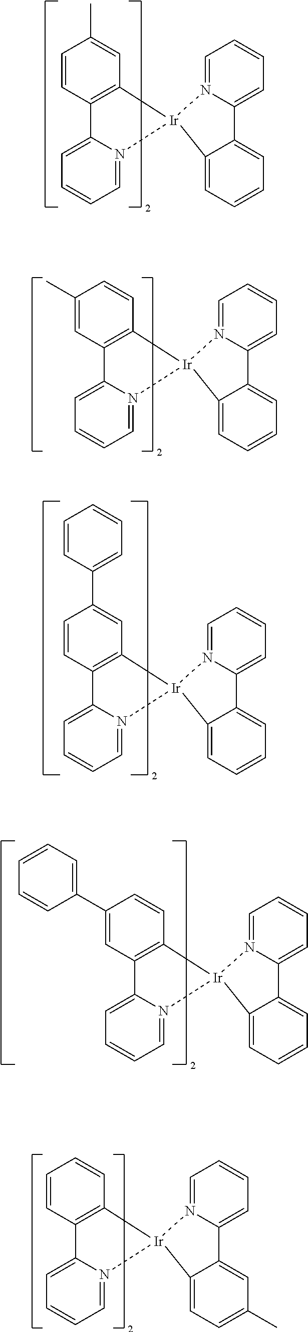

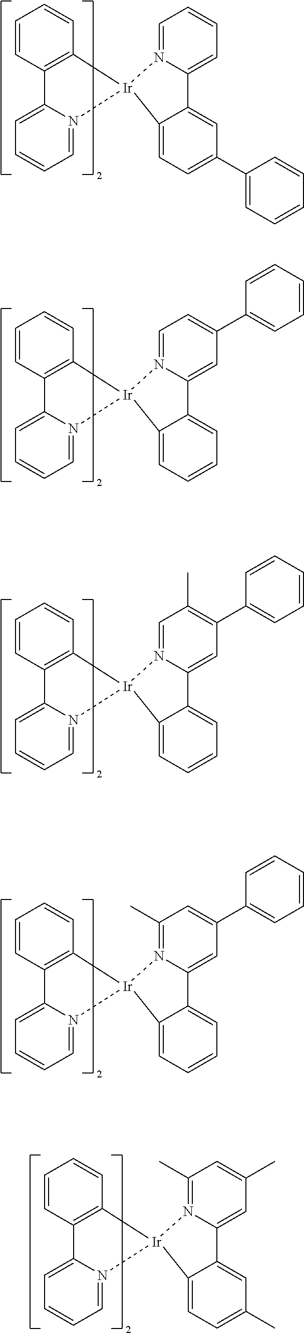

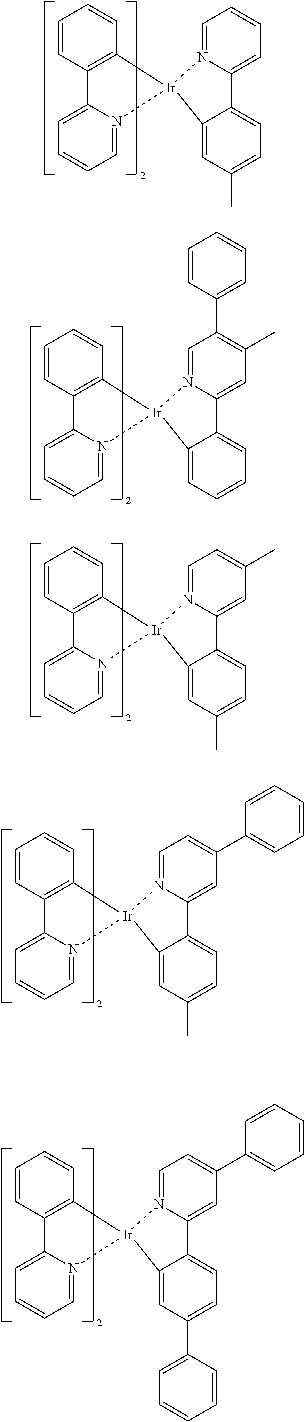

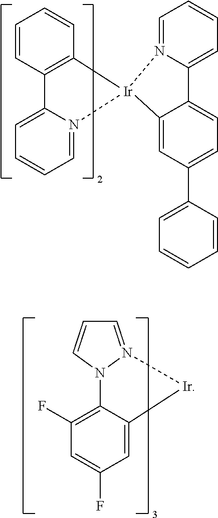

### Hole Blocking Layer

The organic light emitting device according to the present disclosure can include a hole blocking layer on the light emitting layer, if necessary.

The hole blocking layer is a layer provided between the electron transport layer and the light emitting layer in order to prevent the holes injected in the anode from being transferred to the electron transport layer without being recombined in the light emitting layer, which can also be referred to as a hole inhibition layer or a hole stopping layer. The hole blocking layer is preferably a material having the large ionization energy.

### Electron Transport Layer

The organic light emitting device according to the present disclosure can include an electron transport layer on the light emitting layer (or the hole blocking layer), if necessary.

The electron transport layer is a layer that receives the electrons from the cathode or the electron injection layer formed on the cathode and transports the electrons to the light emitting layer, and that suppress the transfer of holes from the light emitting layer, and an electron transport material is suitably a material which can receive well injection of electrons from a cathode and transfer the electrons to a light emitting layer, and has a large mobility for electrons.

Specific examples of the electron transport material include: an Al complex of 8-hydroxyquinoline; a complex including Alq₃; an organic radical compound; a hydroxyflavone-metal complex, and the like, but are not limited thereto. The electron transport layer can be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

### Electron Injection Layer

The organic light emitting device according to the present disclosure can further include an electron injection layer on the light emitting layer (or on the electron transport layer, if the electron transport layer exists), if necessary.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film.

Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

### Organic Light Emitting Device

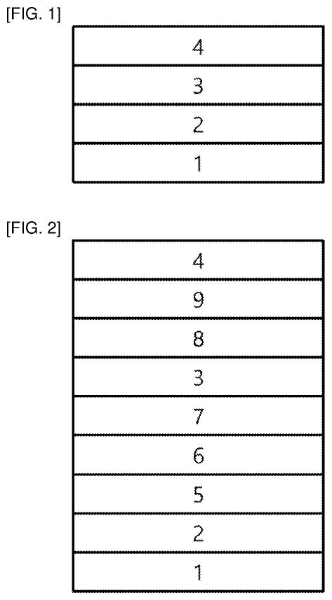

The structure of the organic light emitting device according to the present disclosure is illustrated in FIGS. 1 and 2. FIG. 1 shows an example of an organic light emitting device comprising a substrate 1, an anode 2, a light emitting layer 3, and a cathode 4. FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

The organic light emitting device according to the present disclosure can be manufactured by sequentially stacking the above-described structures. In this case, the organic light emitting device can be manufactured by depositing a metal, metal oxides having conductivity, or an alloy thereof on the substrate by using a PVD (physical vapor deposition) method such as a sputtering method or an e-beam evaporation method to form the anode, forming the respective layers described above thereon, and then depositing a material that can be used as the cathode thereon. In addition to such a method, the organic light emitting device can be manufactured by sequentially depositing from the cathode material to the anode material on a substrate in the reverse order of the above-mentioned configuration (WO 2003/012890). Further, the light emitting layer can be formed by subjecting hosts and dopants to a vacuum deposition method and a solution coating method. Herein, the solution coating method means a spin coating, a dip coating, a doctor blading, an inkjet printing, a screen printing, a spray method, a roll coating, or the like, but is not limited thereto.

Meanwhile, the organic light emitting device according to the present disclosure can be a bottom emission device, a top emission device, or a double-sided light emitting device, and particularly, can be a bottom emission device that requires relatively high luminous efficiency.

Below, preferable embodiments are presented to assist in the understanding of the present disclosure. The following examples are only provided for a better understanding of the present disclosure, and is not intended to limit the content of the present disclosure.

### Synthesis Example 1-1

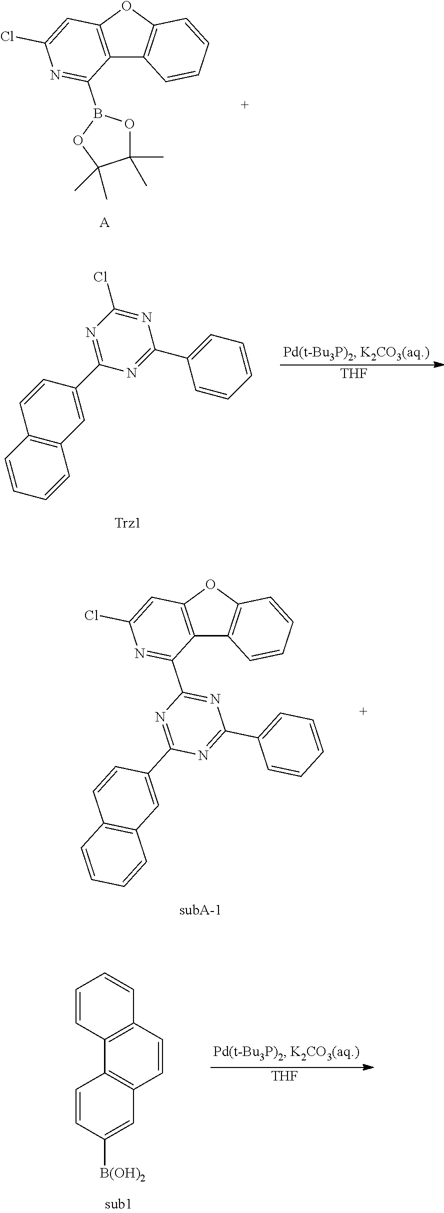

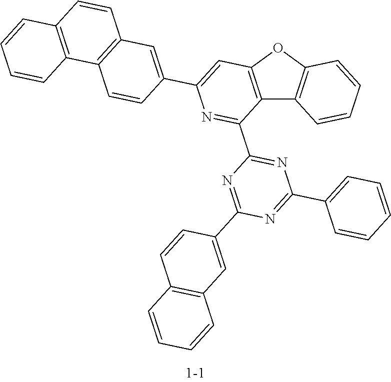 [SMILES: c1ccc(-c2nc(-c3ccc4ccccc4c3)nc(-c3nc(-c4ccc5c(ccc6ccccc65)c4)cc4oc5ccccc5c34)n2)cc1]

Compound A (15 g, 45.5 mmol) and Compound Trz1 (15.2 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.9 g of Compound subA-1. (Yield: 63%, MS: [M+H]⁺=485)

Compound subA-1 (15 g, 30.9 mmol) and Compound sub1 (7.2 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 11.6 g of Compound 1-1. (Yield: 60%, MS: [M+H]⁺=627)

### Synthesis Example 1-2

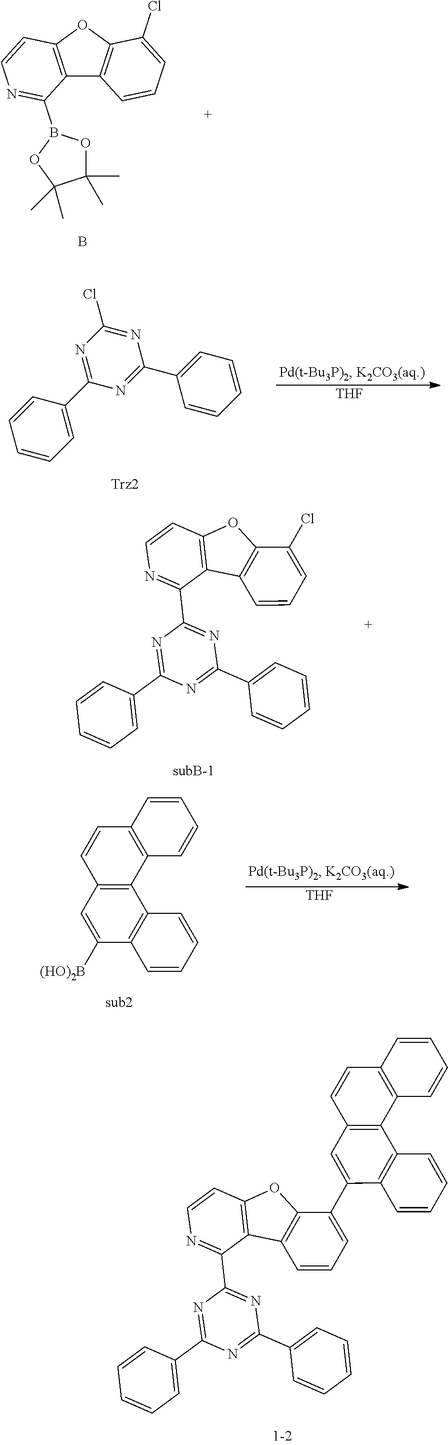

Compound B (15 g, 45.5 mmol) and Compound Trz2 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.6 g of Compound subB-1. (Yield: 69%, MS: [M+H]⁺=435)

Compound subB-1 (15 g, 34.5 mmol) and Compound sub2 (9.9 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.5 g of Compound 1-2. (Yield: 67%, MS: [M+H]⁺=627)

### Synthesis Example 1-3

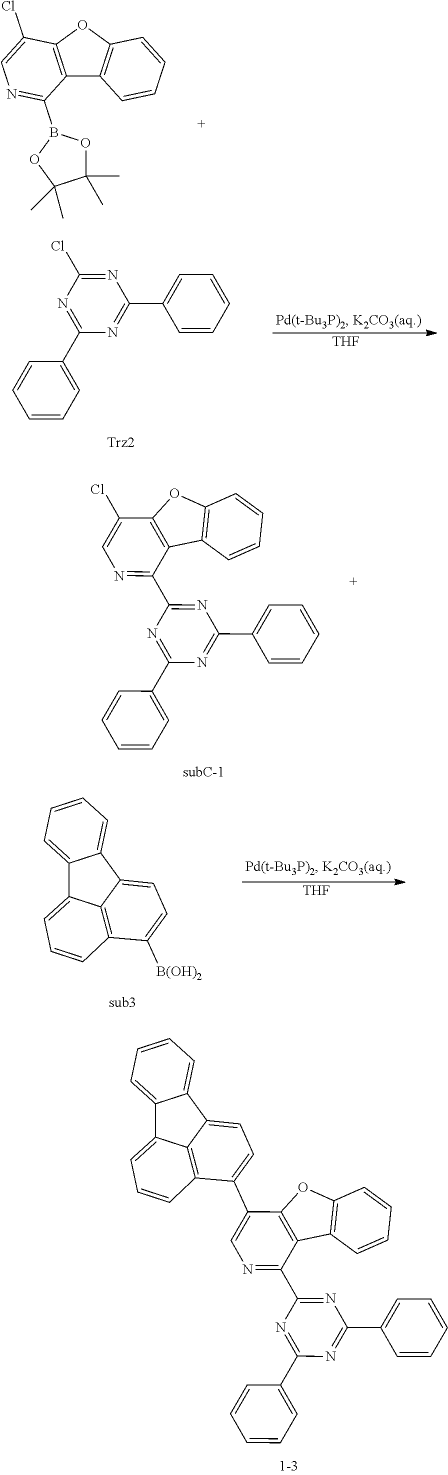

### C

Compound C (15 g, 45.5 mmol) and Compound Trz2 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.6 g of Compound subC-1. (Yield: 64%, MS: [M+H]⁺=435) Compound subC-1 (15 g, 34.5 mmol) and Compound sub3 (8.9 g, 36.2 mmol) were added to 300 ml of THF, and the mixture was stirred and refluxed.

Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.1 g of Compound 1-3. (Yield: 68%, MS: [M+H]⁺=601)

### Synthesis Example 1-4

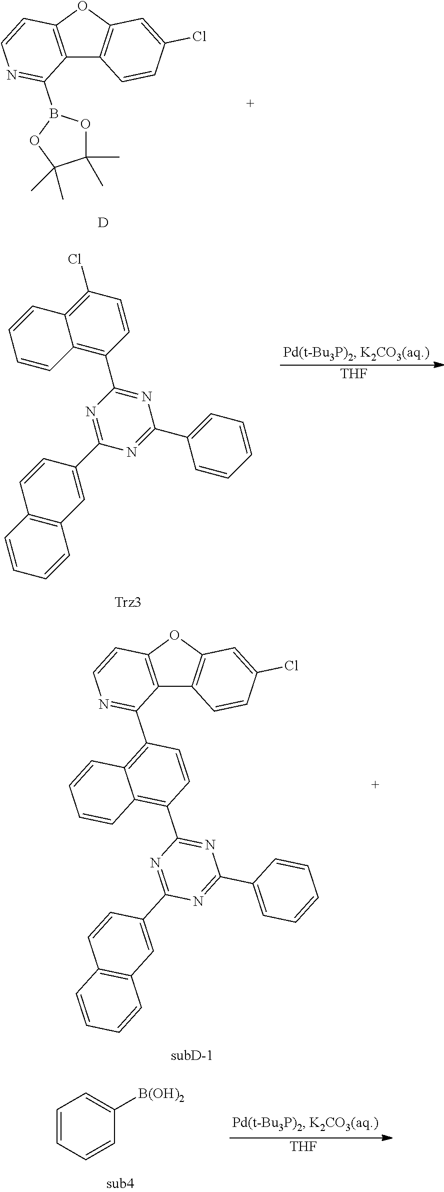

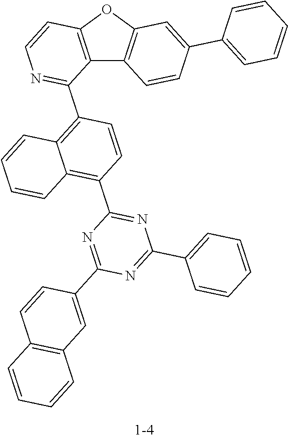 [SMILES: c1ccc(-c2ccc3c(c2)oc2ccnc(-c4ccc(-c5nc(-c6ccccc6)nc(-c6ccc7ccccc7c6)n5)c5ccccc45)c23)cc1]

Compound D (15 g, 45.5 mmol) and Compound Trz3 (21.2 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 21.1 g of Compound subD-1. (Yield: 76%, MS: [M+H]⁺=611)

Compound subD-1 (15 g, 24.5 mmol) and Compound sub4 (3.1 g, 25.8 mmol) were added to 300 ml of THF, and the mixture was stirred and refluxed. Then, potassium carbonate (10.2 g, 73.6 mmol) was dissolved in 100 ml of water and added thereto, and the mixture was sufficiently stirred and then bis(tri-tert-butylphosphine)palladium(0) (0.1 g, 0.2 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.8 g of Compound 1-4. (Yield: 80%, MS: [M+H]⁺=653)

### Synthesis Example 1-5

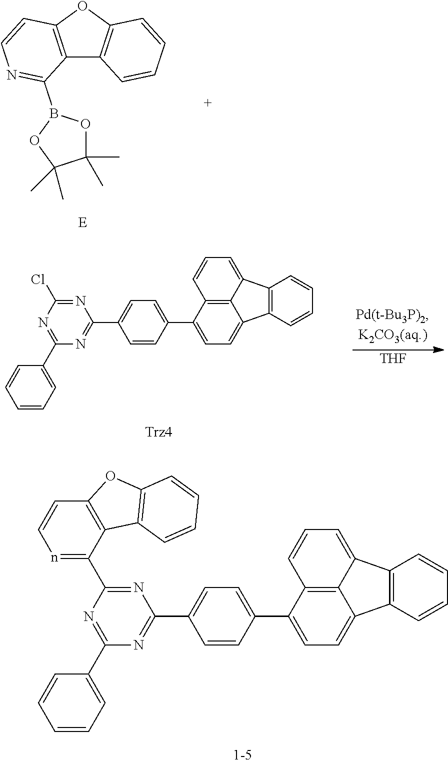

Compound E (15 g, 50.8 mmol) and Compound Trz4 (25 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 20.4 g of Compound 1-5. (Yield: 67%, MS: [M+H]⁺=601)

### Synthesis Example 1-6

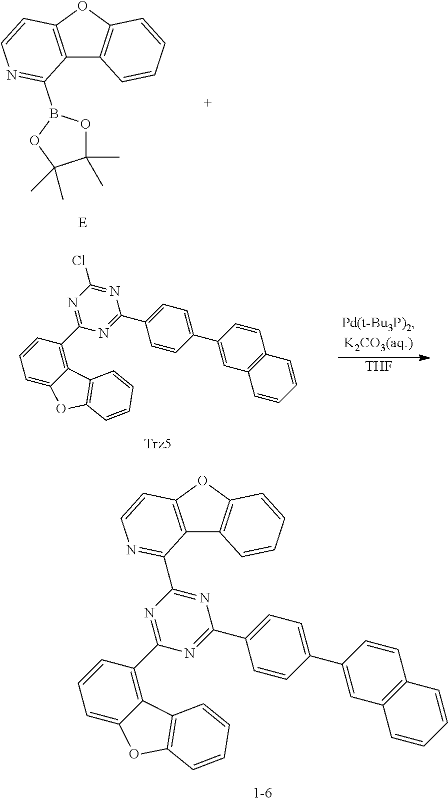

Compound E (15 g, 50.8 mmol) and Compound Trz5 (25.8 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 20.4 g of Compound 1-6. (Yield: 65%, MS: [M+H]⁺=617)

### Synthesis Example 1-7

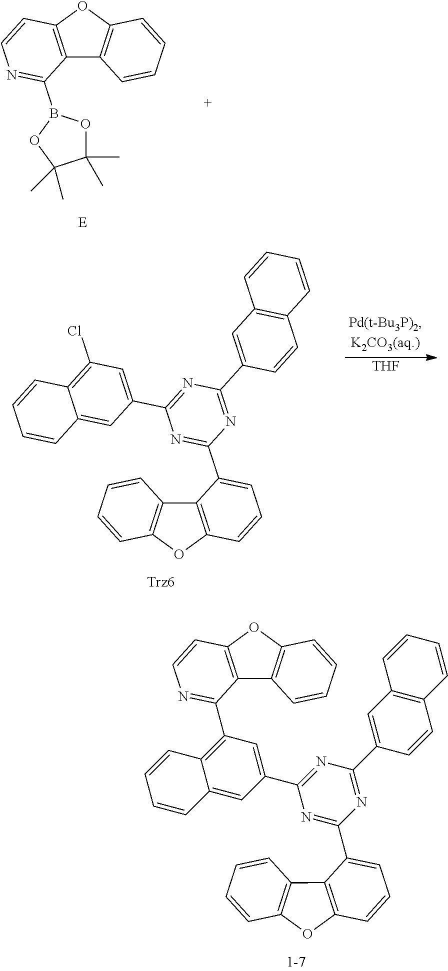

Compound E (15 g, 50.8 mmol) and Compound Trz6 (28.5 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled.

This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 20.7 g of Compound 1-7. (Yield: 61%, MS: [M+H]⁺=667)

### Synthesis Example 1-8

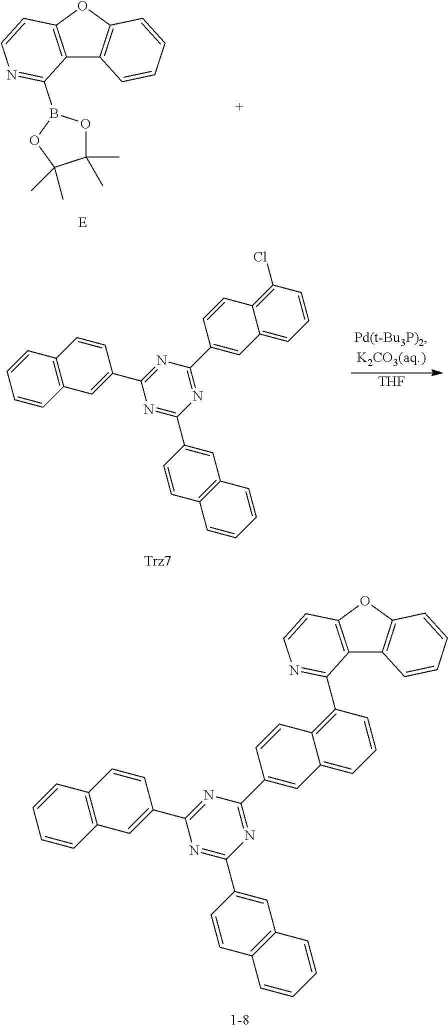

Compound E (15 g, 50.8 mmol) and Compound Trz7 (26.4 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 24.2 g of Compound 1-8. (Yield: 76%, MS: [M+H]⁺=627)

### Synthesis Example 1-9

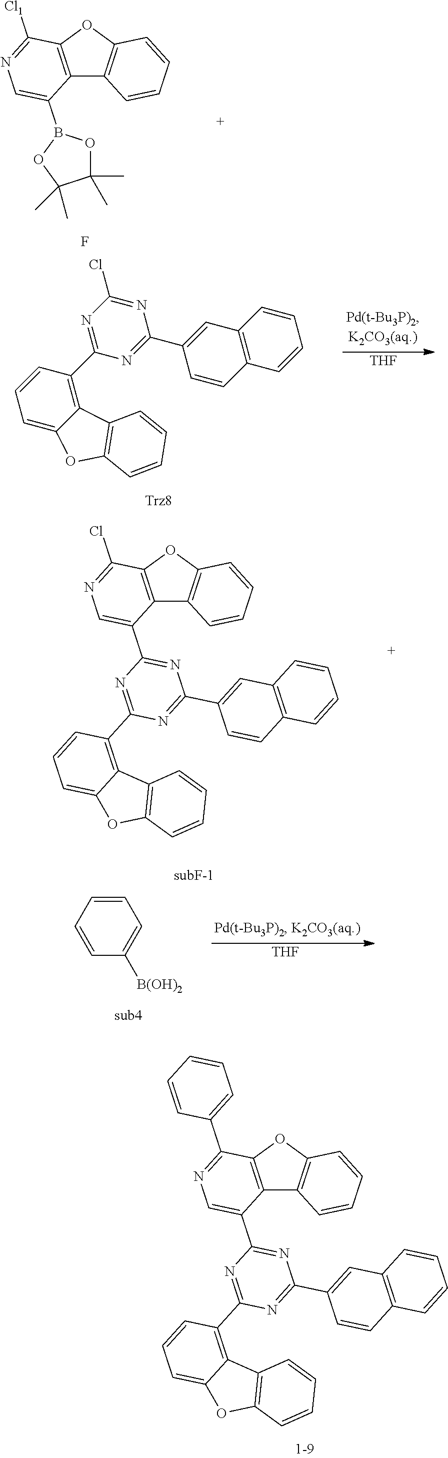

Compound F (15 g, 45.5 mmol) and Compound Trz8 (19.5 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 17 g of Compound subF-1. (Yield: 65%, MS: [M+H]⁺=575)

Compound subF-1 (15 g, 26.1 mmol) and Compound sub4 (3.3 g, 27.4 mmol) were added to 300 ml of THF, and the mixture was stirred and refluxed. Then, potassium carbonate (10.8 g, 78.3 mmol) was dissolved in 100 ml of water and added thereto, and the mixture was sufficiently stirred and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.9 g of Compound 1-9. (Yield: 80%, MS: [M+H]⁺=617)

### Synthesis Example 1-10

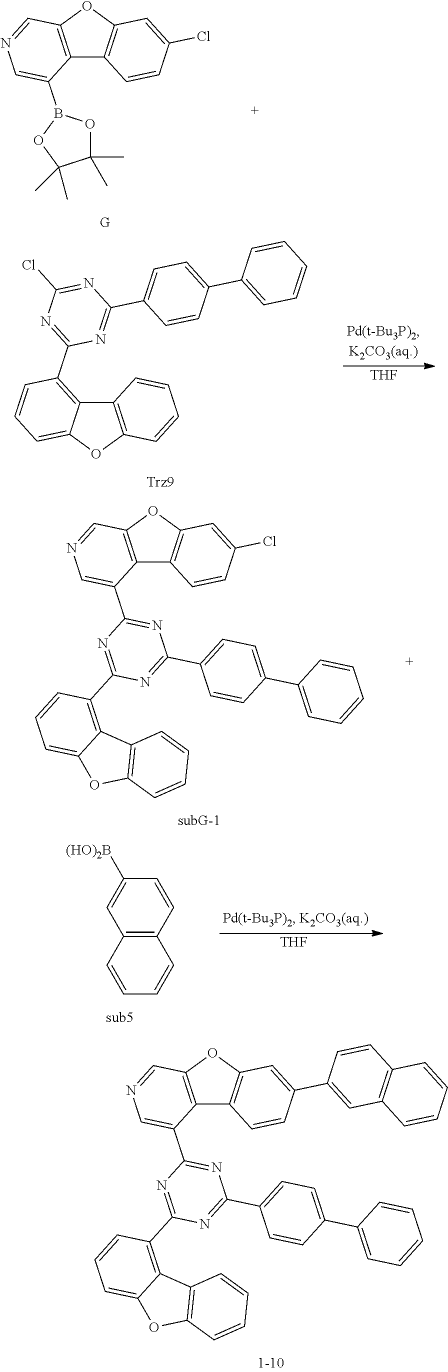

Compound G (15 g, 45.5 mmol) and Compound Trz9 (20.7 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 21.9 g of Compound subG-1. (Yield: 80%, MS: [M+H]⁺=601)

Compound subG-1 (15 g, 25 mmol) and Compound sub5 (4.5 g, 26.2 mmol) were added to 300 ml of THF, and the mixture was stirred and refluxed. Then, potassium carbonate (10.3 g, 74.9 mmol) was dissolved in 100 ml of water and added thereto, and the mixture was sufficiently stirred and then bis(tri-tert-butylphosphine)palladium(0) (0.1 g, 0.2 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13 g of Compound 1-10. (Yield: 75%, MS: [M+H]⁺=693)

### Synthesis Example 1-11

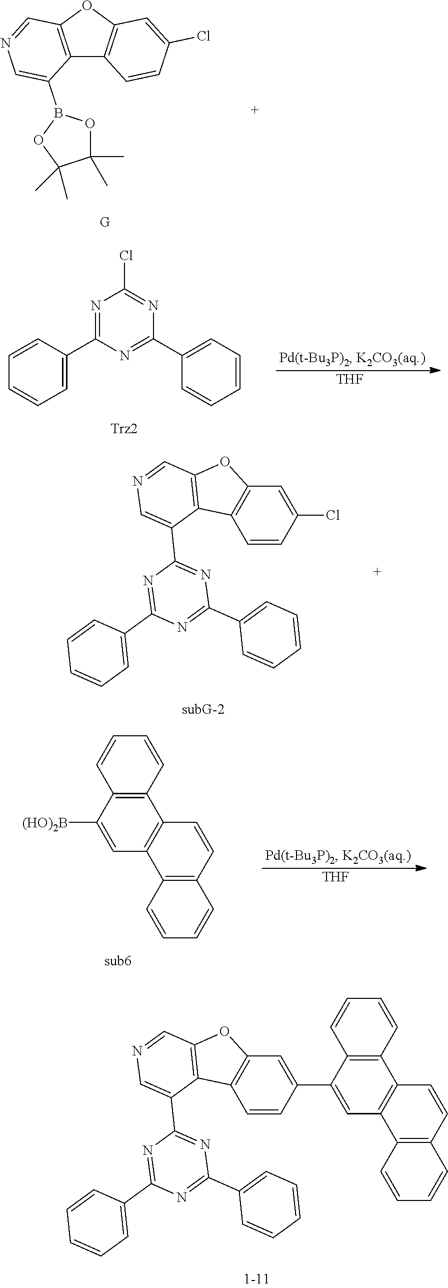

Compound G (15 g, 45.5 mmol) and Compound Trz2 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.8 g of Compound subG-2. (Yield: 70%, MS: [M+H]⁺=435)

Compound subG-2 (15 g, 34.5 mmol) and Compound sub6 (17.5 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14 g of Compound 1-11. (Yield: 65%, MS: [M+H]⁺=627)

### Synthesis Example 1-12

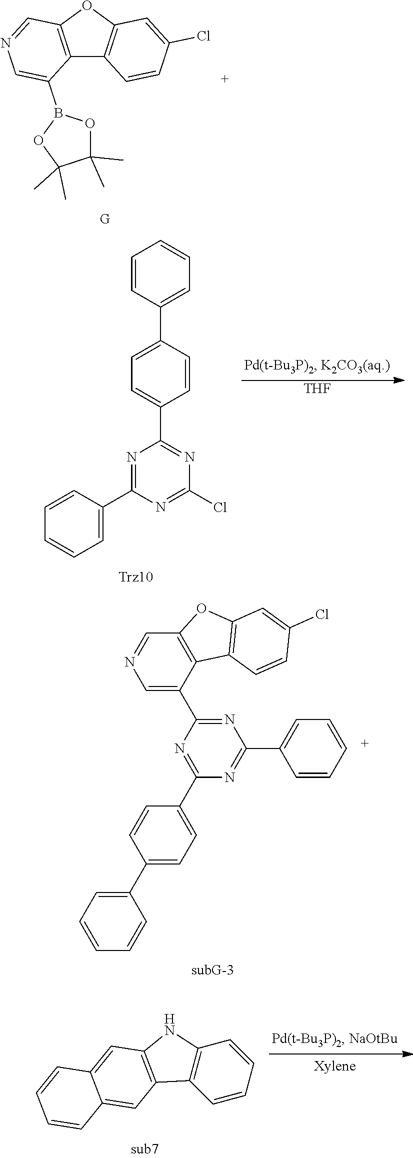 [SMILES: CC1(C)OB(c2cncc3oc4cc(Cl)ccc4c23)OC1(C)C ; Clc1nc(-c2ccccc2)nc(-c2ccc(-c3ccccc3)cc2)n1 ; Clc1ccc2c(c1)oc1cncc(-c3nc(-c4ccccc4)nc(-c4ccc(-c5ccccc5)cc4)n3)c12 ; "THF" ; c1ccc2cc3c(cc2c1)[nH]c1ccccc13]

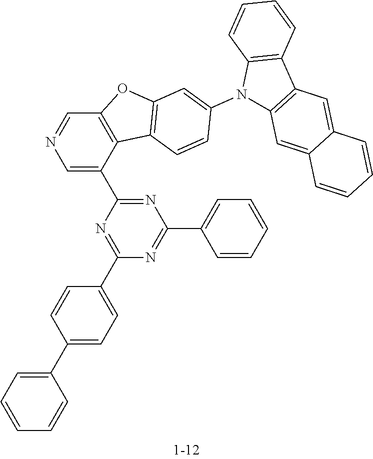 [SMILES: c1ccc(-c2ccc(-c3nc(-c4ccccc4)nc(-c4cncc5oc6cc(-n7c8ccccc8c8cc9ccccc9cc87)ccc6c45)n3)cc2)cc1]

Compound G (15 g, 45.5 mmol) and Compound Trz10 (16.4 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.2 g of Compound subG-3. (Yield: 61%, MS: [M+H]⁺=511)

Compound subG-3 (10 g, 19.6 mmol), Compound sub7 (4.3 g, 20 mmol), and sodium tert-butoxide (2.4 g, 25.4 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 9.5 g of Compound 1-12. (Yield: 70%, MS: [M+H]⁺=692)

### Synthesis Example 1-13

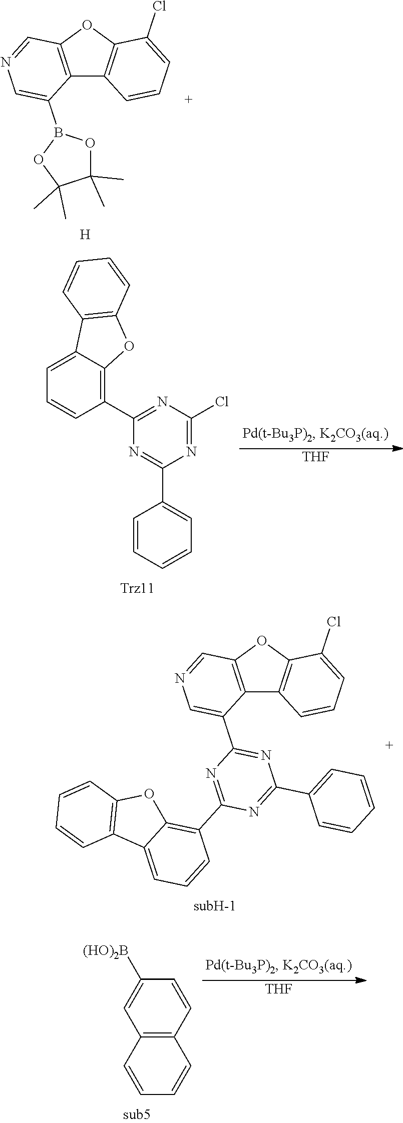

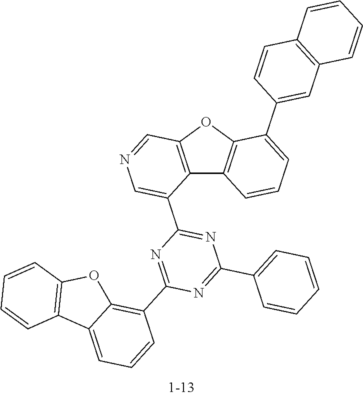 [SMILES: c1ccc(-c2nc(-c3cccc4c3oc3ccccc34)nc(-c3cncc4oc5c(-c6ccc7ccccc7c6)cccc5c34)n2)cc1]

Compound H (15 g, 45.5 mmol) and Compound Trz11 (17.1 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.2 g of Compound subH-1. (Yield: 68%, MS: [M+H]⁺=525)

Compound subH-1 (15 g, 28.6 mmol) and Compound sub5 (5.2 g, 30 mmol) were added to 300 ml of THF, and the mixture was stirred and refluxed. Then, potassium carbonate (11.8 g, 85.7 mmol) was dissolved in 100 ml of water and added thereto, and the mixture was sufficiently stirred and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 10.9 g of Compound 1-13. (Yield: 62%, MS: [M+H]⁺=617)

### Synthesis Example 1-14

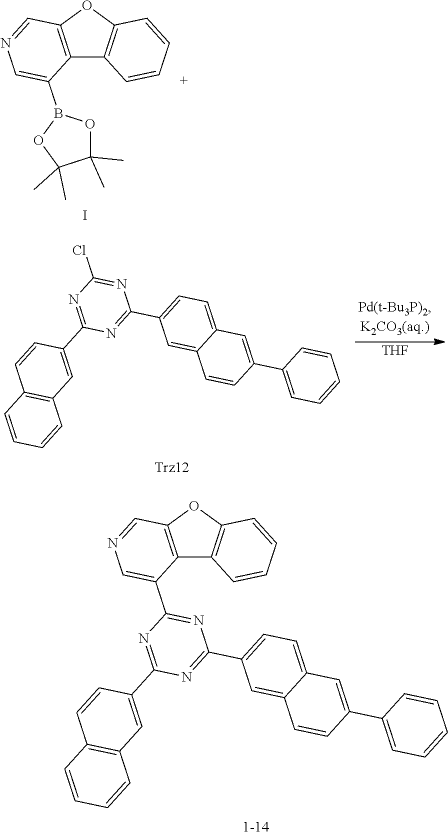

Compound I (15 g, 50.8 mmol) and Compound Trz12 (23.7 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 17.6 g of Compound 1-14. (Yield: 60%, MS: [M+H]⁺=577)

### Synthesis Example 1-15

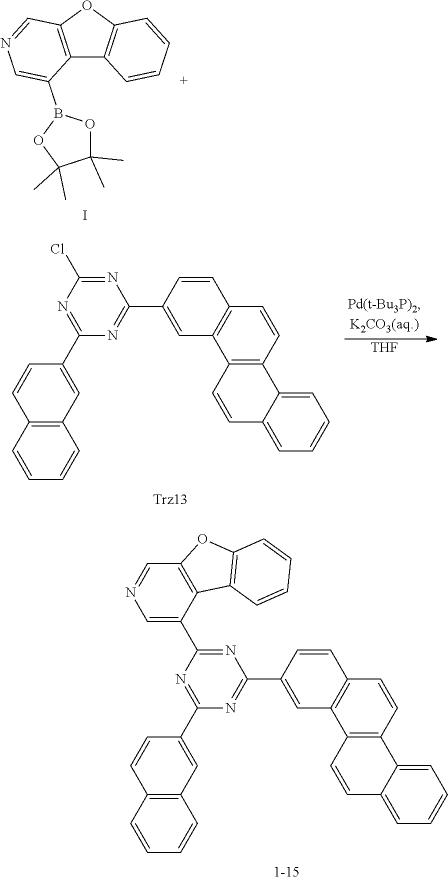

Compound I (15 g, 50.8 mmol) and Compound Trz13 (25 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 21.7 g of Compound 1-15. (Yield: 71%, MS: [M+H]⁺=601)

### Synthesis Example 1-16

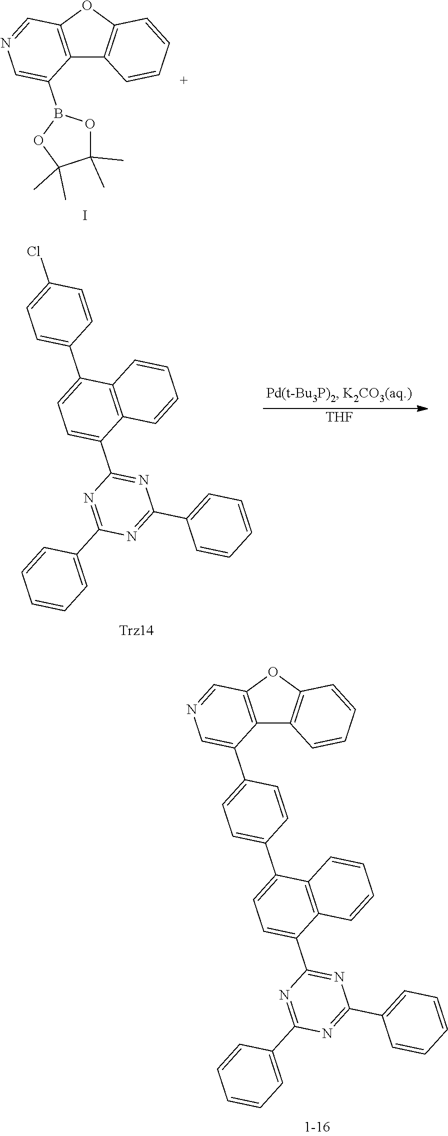

Compound I (15 g, 50.8 mmol) and Compound Trz14 (25.1 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 21.4 g of Compound 1-16. (Yield: 70%, MS: [M+H]⁺=603)

### Synthesis Example 1-17

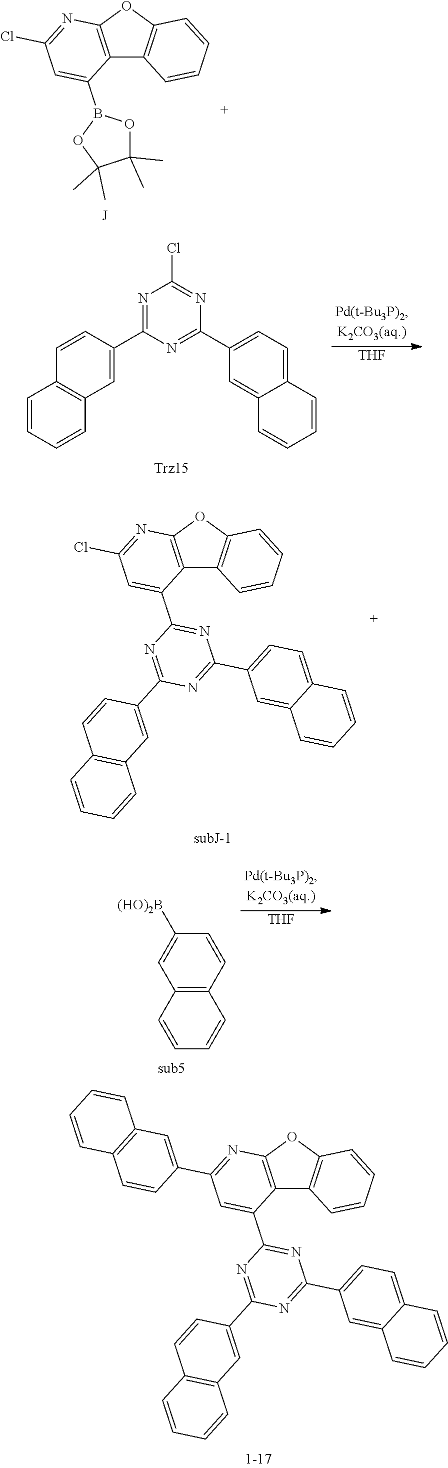

Compound J (15 g, 45.5 mmol) and Compound Trz15 (17.6 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.6 g of Compound subJ-1. (Yield: 64%, MS: [M+H]⁺=535)

Compound subJ-1 (15 g, 28 mmol) and Compound sub5 (5.1 g, 29.4 mmol) were added to 300 ml of THF, and the mixture was stirred and refluxed. Then, potassium carbonate (11.6 g, 84.1 mmol) was dissolved in 100 ml of water and added thereto, and the mixture was sufficiently stirred and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.7 g of Compound 1-17. (Yield: 78%, MS: [M+H]⁺=627)

### Synthesis Example 1-18

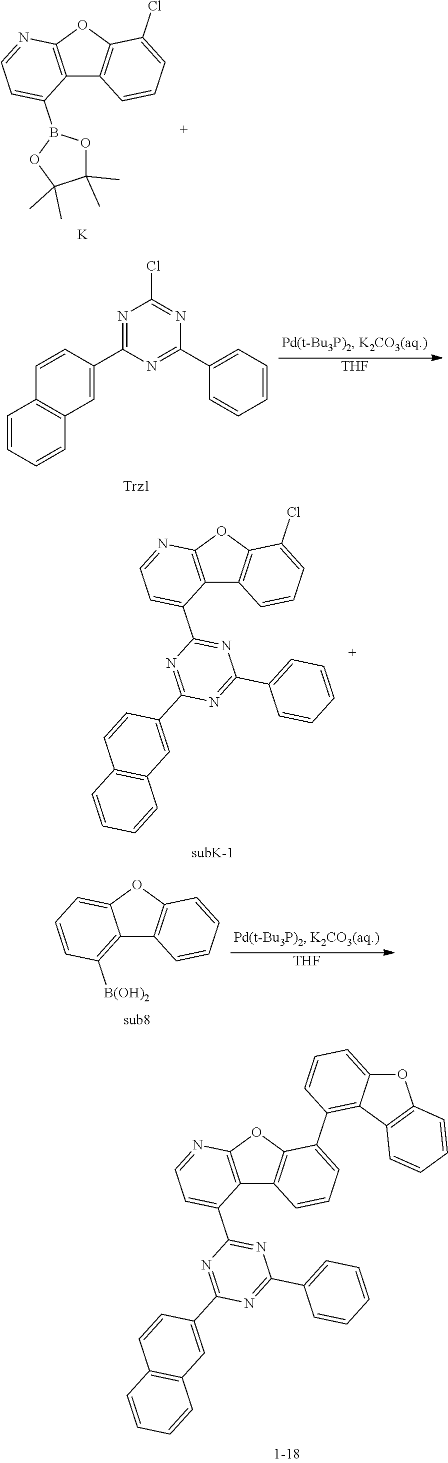

Compound K (15 g, 45.5 mmol) and Compound Trz1 (15.2 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.9 g of Compound subK-1. (Yield: 63%, MS: [M+H]⁺=485)

Compound subK-1 (15 g, 30.9 mmol) and Compound sub8 (6.9 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.4 g of Compound 1-18. (Yield: 65%, MS: [M+H]⁺=617)

### Synthesis Example 1-19

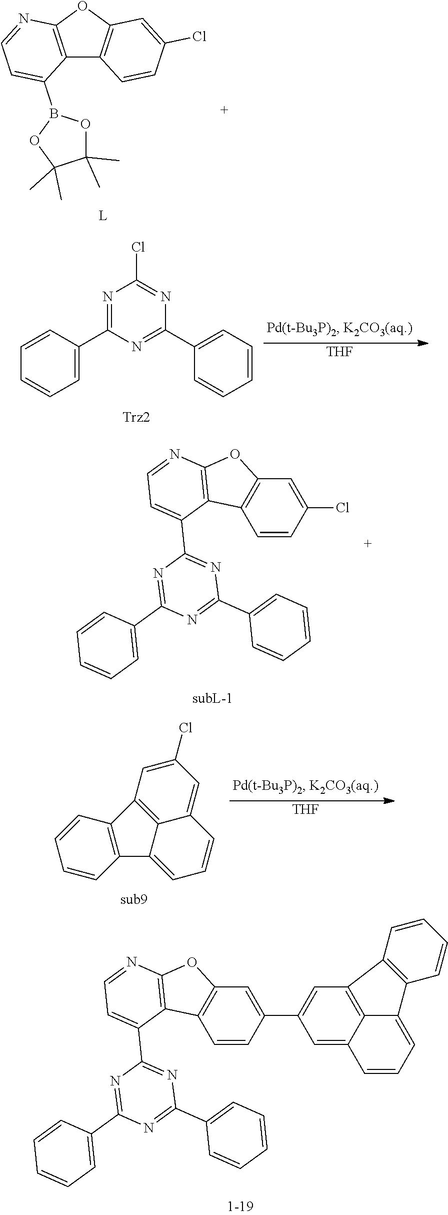

Compound L (15 g, 45.5 mmol) and Compound Trz2 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.6 g of Compound subL-1. (Yield: 69%, MS: [M+H]⁺=435)

Compound subL-1 (15 g, 34.5 mmol) and Compound sub9 (8.9 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.2 g of Compound 1-19. (Yield: 64%, MS: [M+H]⁺=601)

### Synthesis Example 1-20

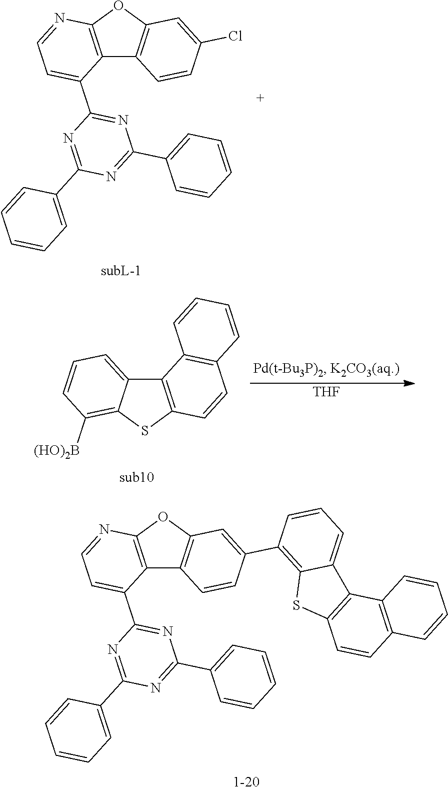 [SMILES: Clc1ccc2c(c1)oc1nccc(-c3nc(-c4ccccc4)nc(-c4ccccc4)n3)c12 ; OB(O)c1cccc2c1sc1ccc3ccccc3c12 ; c1ccc(-c2nc(-c3ccccc3)nc(-c3ccnc4oc5cc(-c6cccc7c6sc6ccc8ccccc8c67)ccc5c34)n2)cc1 ; "THF"]

Compound subL-1 (15 g, 34.5 mmol) and Compound sub10 (10.1 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.4 g of Compound 1-20. (Yield: 66%, MS: [M+H]⁺=633)

### Synthesis Example 1-21

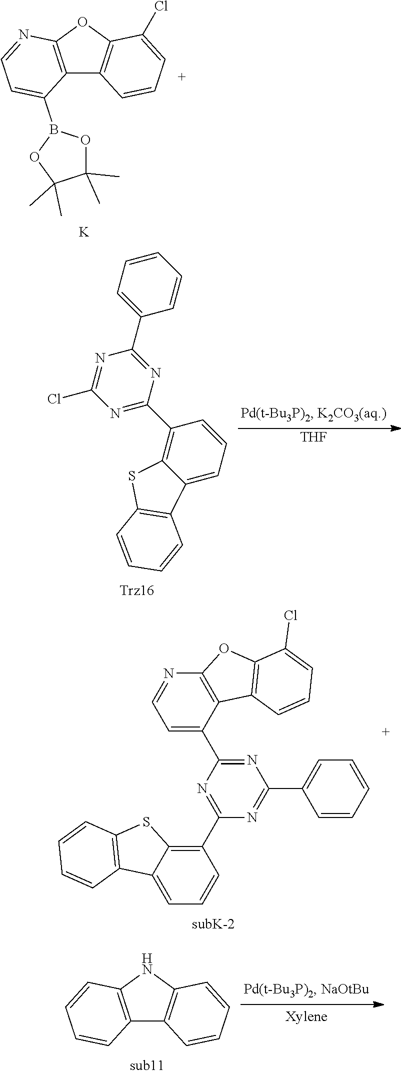

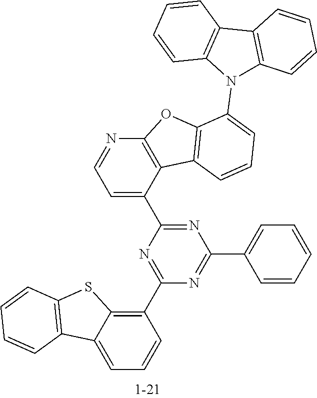 [SMILES: c1ccc(-c2nc(-c3cccc4c3sc3ccccc34)nc(-c3ccnc4oc5c(-n6c7ccccc7c7ccccc76)cccc5c34)n2)cc1]

Compound K (15 g, 45.5 mmol) and Compound Trz16 (17.9 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.7 g of Compound subK-2. (Yield: 68%, MS: [M+H]⁺=541)

Compound subK-2 (10 g, 18.5 mmol), Compound sub11 (3.2 g, 18.9 mmol), and sodium tert-butoxide (2.3 g, 24 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 7.8 g of Compound 1-21. (Yield: 63%, MS: [M+H]⁺=672)

### Synthesis Example 1-22

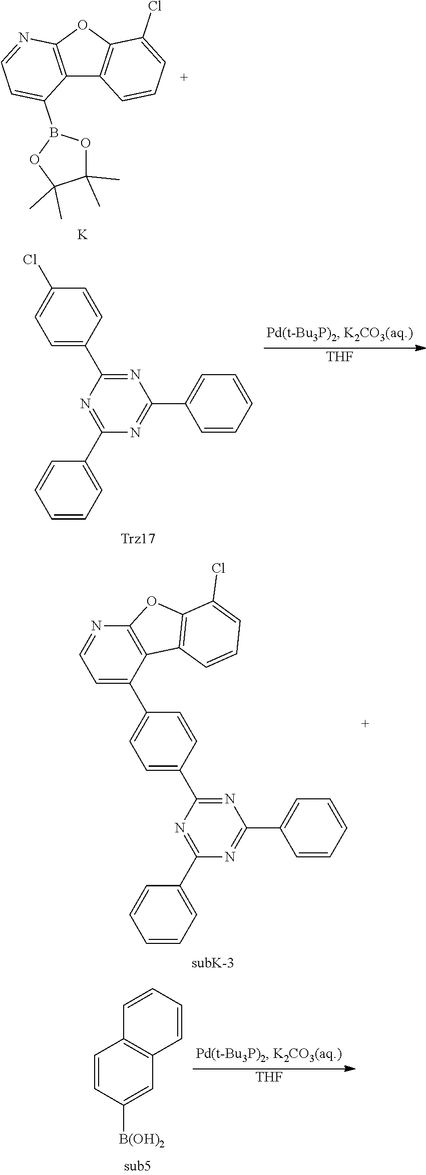

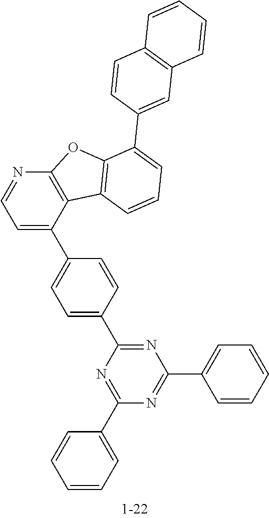 [SMILES: c1ccc(-c2nc(-c3ccccc3)nc(-c3ccc(-c4ccnc5oc6c(-c7ccc8ccccc8c7)cccc6c45)cc3)n2)cc1]

Compound K (15 g, 45.5 mmol) and Compound Trz17 (16.4 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.3 g of Compound subK-3. (Yield: 66%, MS: [M+H]⁺=511)

Compound subK-3 (15 g, 29.4 mmol) and Compound sub5 (5.3 g, 30.8 mmol) were added to 300 ml of THF, and the mixture was stirred and refluxed. Then, potassium carbonate (12.2 g, 88.1 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.8 g of Compound 1-22. (Yield: 78%, MS: [M+H]⁺=603)

### Synthesis Example 1-23

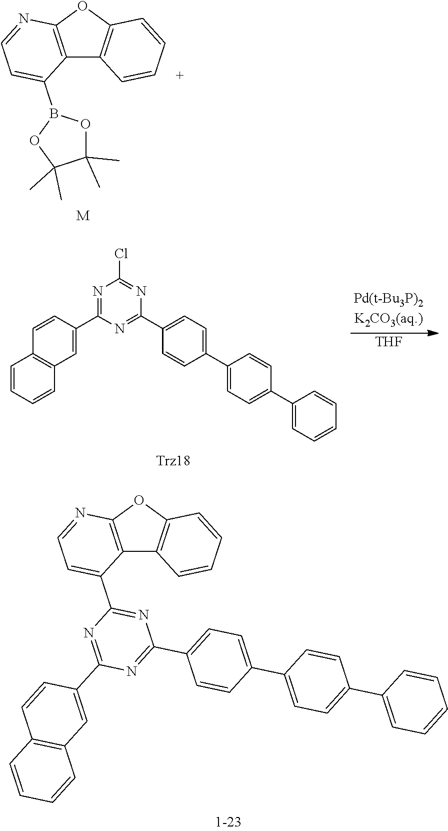

Compound M (15 g, 50.8 mmol) and Compound Trz18 (25.1 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.9 g of Compound 1-23. (Yield: 65%, MS: [M+H]⁺=603)

### Synthesis Example 1-24

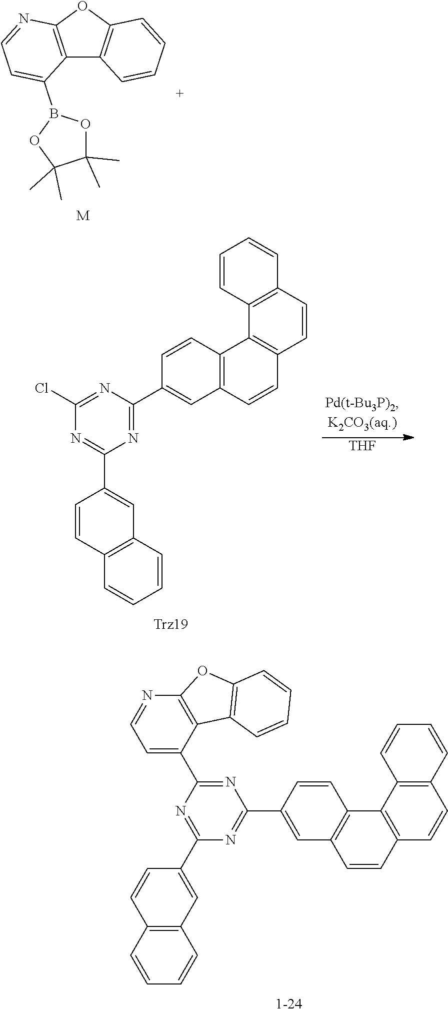

Compound M (15 g, 50.8 mmol) and Compound Trz19 (25 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 20.4 g of Compound 1-24. (Yield: 67%, MS: [M+H]⁺=601)

### Synthesis Example 1-25

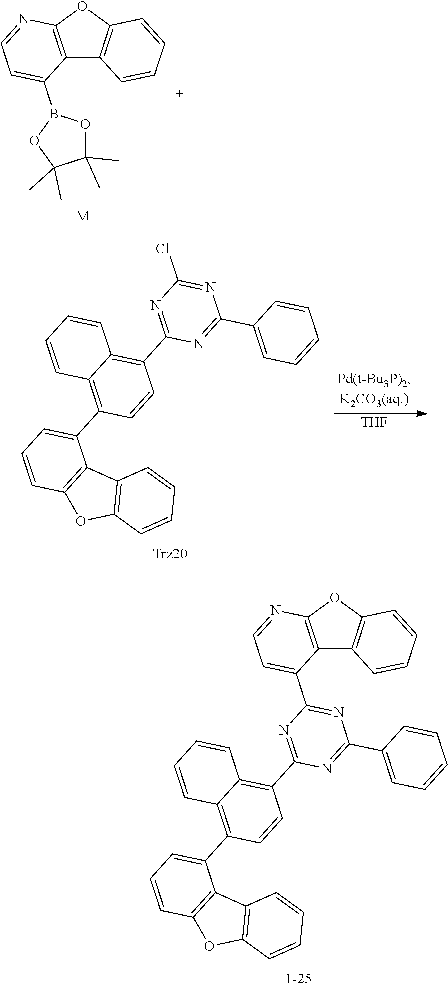

Compound M (15 g, 50.8 mmol) and Compound Trz20 (25.8 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.7 g of Compound 1-25. (Yield: 63%, MS: [M+H]⁺=617)

### Synthesis Example 1-26

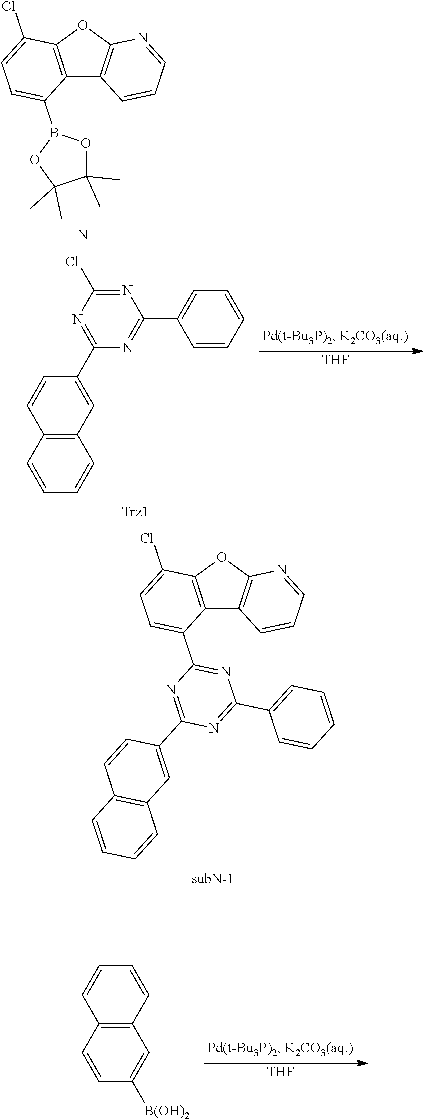

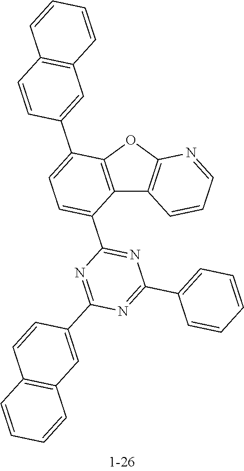 [SMILES: c1ccc(-c2nc(-c3ccc4ccccc4c3)nc(-c3ccc(-c4ccc5ccccc5c4)c4oc5ncccc5c34)n2)cc1]

Compound N (15 g, 45.5 mmol) and Compound Trz1 (15.2 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.9 g of Compound subN-1. (Yield: 72%, MS: [M+H]⁺=485)

Compound subN-1 (15 g, 30.9 mmol) and Compound sub5 (5.6 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.7 g of Compound 1-26. (Yield: 71%, MS: [M+H]⁺=577)

### Synthesis Example 1-27

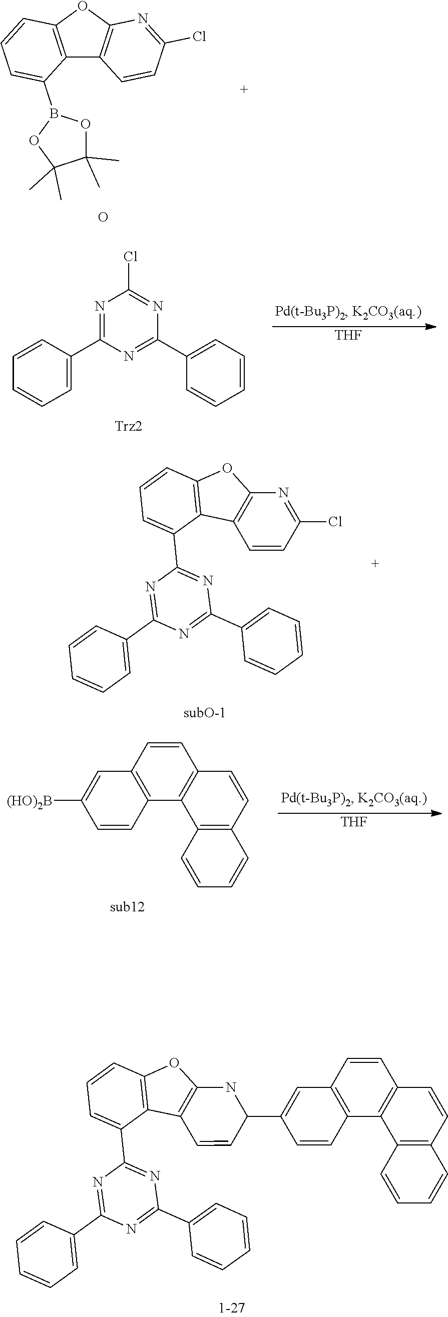

Compound O (15 g, 45.5 mmol) and Compound Trz2 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15 g of Compound subO-1. (Yield: 76%, MS: [M+H]⁺=435)

Compound subO-1 (15 g, 34.5 mmol) and Compound sub12 (9.9 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.8 g of Compound 1-27. (Yield: 73%, MS: [M+H]⁺=627)

### Synthesis Example 1-28

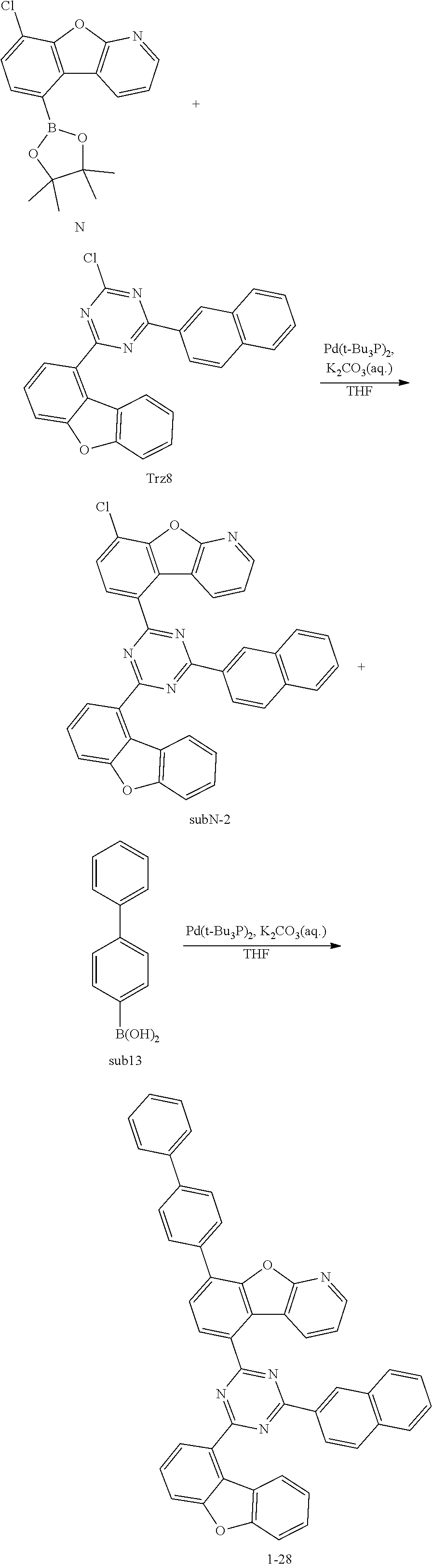

Compound N (15 g, 45.5 mmol) and Compound Trz8 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 20.4 g of Compound subN-2. (Yield: 78%, MS: [M+H]⁺=575)

Compound subN-2 (15 g, 26.1 mmol) and Compound sub13 (5.4 g, 27.4 mmol) were added to 300 ml of THF, and the mixture was stirred and refluxed. Then, potassium carbonate (10.8 g, 78.3 mmol) was dissolved in 100 ml of water and added thereto, and the mixture was sufficiently stirred and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 10.8 g of Compound 1-28. (Yield: 60%, MS: [M+H]⁺=693)

### Synthesis Example 1-29

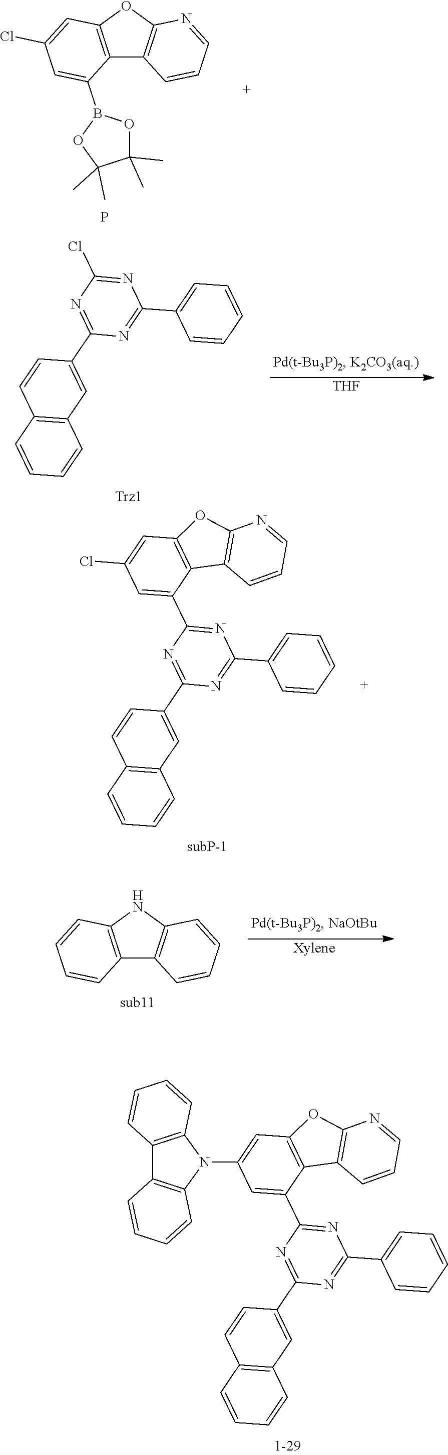

Compound P (15 g, 45.5 mmol) and Compound Trz1 (15.2 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.7 g of Compound subP-1. (Yield: 62%, MS: [M+H]⁺=485)

Compound subP-1 (10 g, 20.6 mmol), Compound sub11 (3.5 g, 21 mmol), and sodium tert-butoxide (2.6 g, 26.8 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 4 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 6.5 g of Compound 1-29. (Yield: 51%, MS: [M+H]⁺=616)

### Synthesis Example 1-30

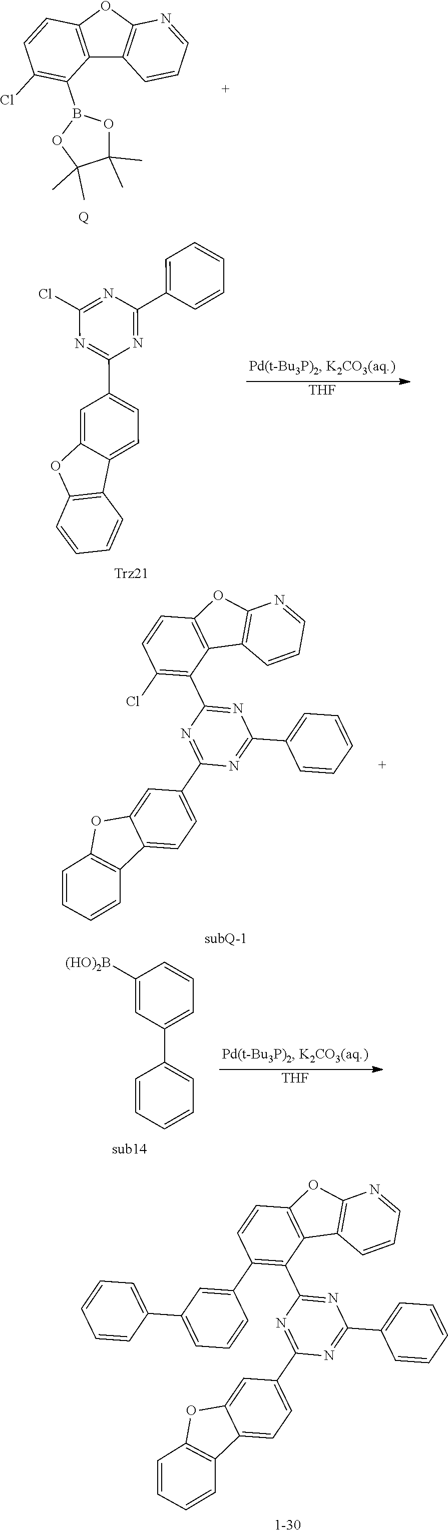

Compound Q (15 g, 45.5 mmol) and Compound Trz21 (17.1 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.5 g of Compound subQ-1. (Yield: 69%, MS: [M+H]⁺=525)

Compound subQ-1 (15 g, 28.6 mmol) and Compound sub14 (5.9 g, 30 mmol) were added to 300 ml of THF, and the mixture was stirred and refluxed. Then, potassium carbonate (11.8 g, 85.7 mmol) was dissolved in 100 ml of water and added thereto, and the mixture was sufficiently stirred and then bis(tri-tert-butylphosphine)palladium(0) (0.1 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.7 g of Compound 1-30. (Yield: 80%, MS: [M+H]⁺=643)

### Synthesis Example 1-31

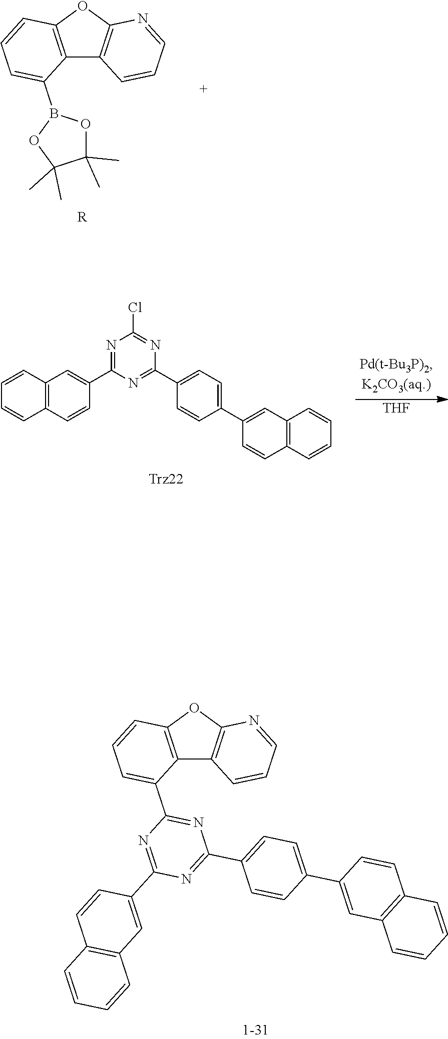

Compound R (15 g, 50.8 mmol) and Compound Trz22 (23.7 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.7 g of Compound 1-31. (Yield: 64%, MS: [M+H]⁺=577)

### Synthesis Example 1-32

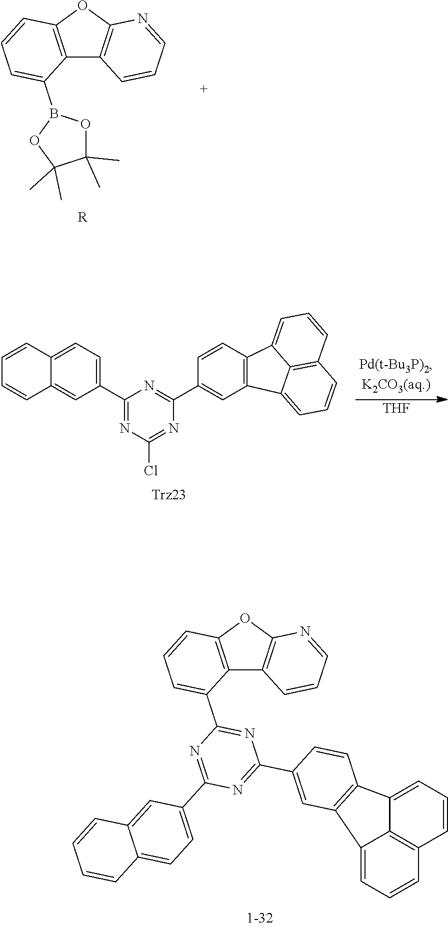

Compound R (15 g, 50.8 mmol) and Compound Trz23 (23.6 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 23.1 g of Compound 1-32. (Yield: 79%, MS: [M+H]⁺=575)

### Synthesis Example 1-33

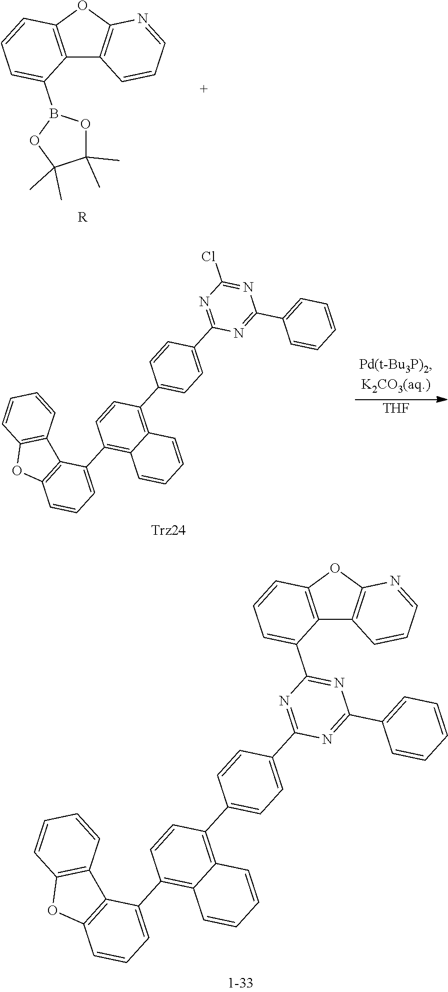

Compound R (15 g, 50.8 mmol) and Compound Trz24 (29.9 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 26 g of Compound 1-33. (Yield: 74%, MS: [M+H]⁺=693)

### Synthesis Example 1-34

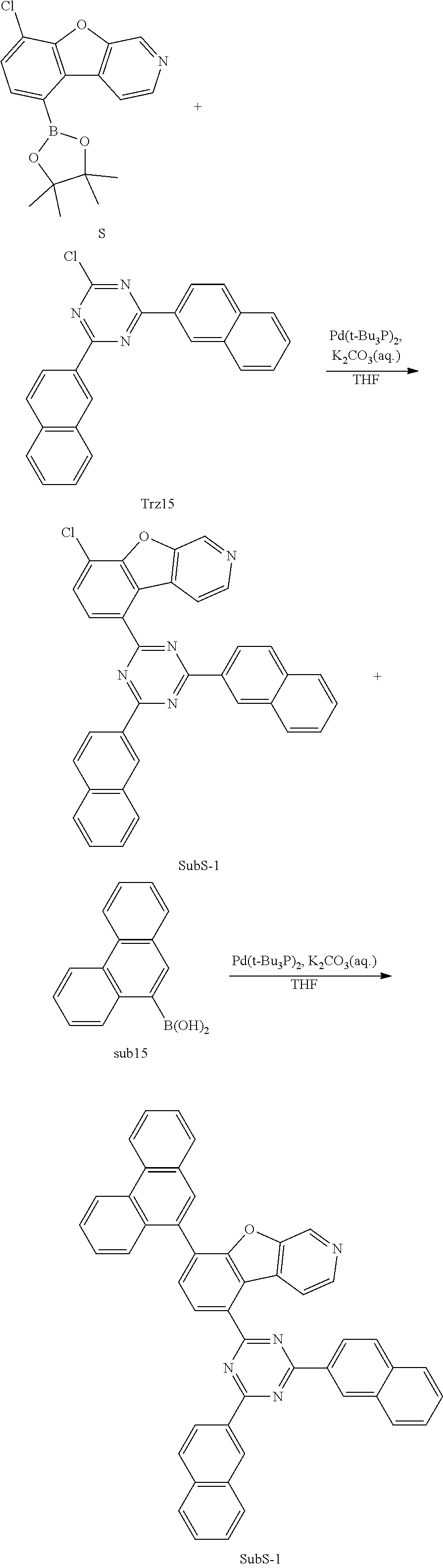

Compound S (15 g, 45.5 mmol) and Compound Trz15 (17.6 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19 g of Compound subS-1. (Yield: 78%, MS: [M+H]⁺=535)

Compound subS-1 (15 g, 28 mmol) and Compound sub15 (6.5 g, 29.4 mmol) were added to 300 ml of THF, and the mixture was stirred and refluxed. Then, potassium carbonate (11.6 g, 84.1 mmol) was dissolved in 100 ml of water and added thereto, and the mixture was sufficiently stirred and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.3 g of Compound 1-34. (Yield: 70%, MS: [M+H]⁺=677)

### Synthesis Example 1-35

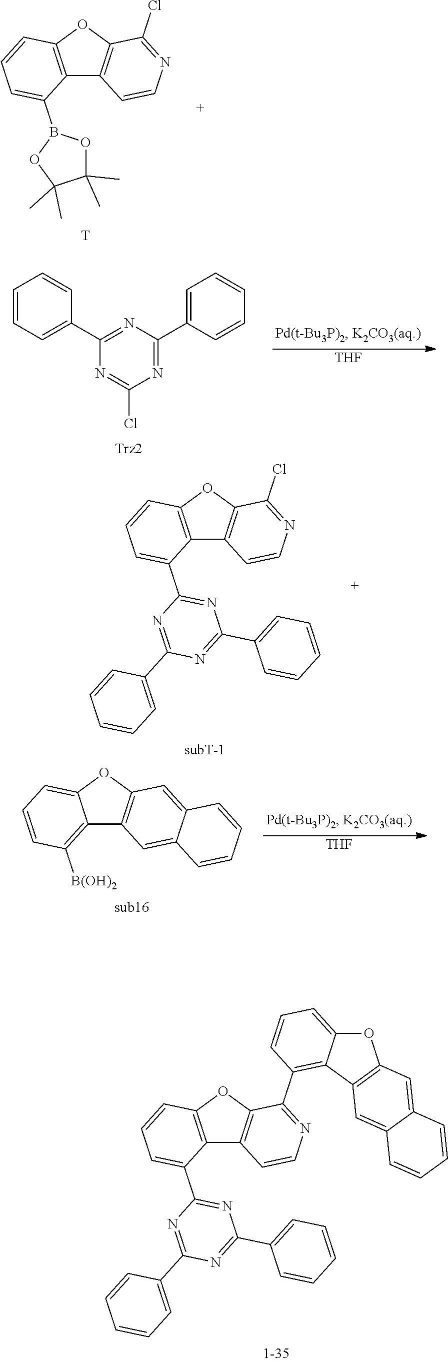

Compound T (15 g, 45.5 mmol) and Compound Trz2 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.4 g of Compound subT-1. (Yield: 73%, MS: [M+H]⁺=435)

Compound subT-1 (15 g, 34.5 mmol) and Compound sub16 (9.5 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 17 g of Compound 1-35. (Yield: 80%, MS: [M+H]⁺=617)

### Synthesis Example 1-36

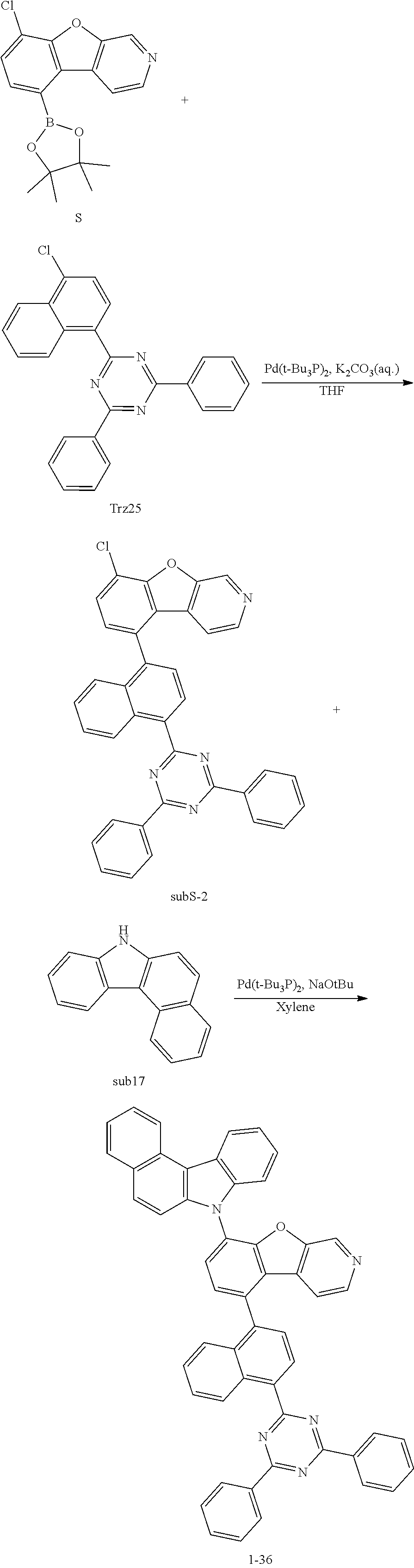

Compound S (15 g, 45.5 mmol) and Compound Trz25 (18.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.6 g of Compound subS-2. (Yield: 77%, MS: [M+H]⁺=561)

Compound subS-2 (10 g, 17.8 mmol), Compound sub17 (4 g, 18.2 mmol), and sodium tert-butoxide (2.2 g, 23.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 7.3 g of Compound 1-36. (Yield: 55%, MS: [M+H]⁺=742)

### Synthesis Example 1-37

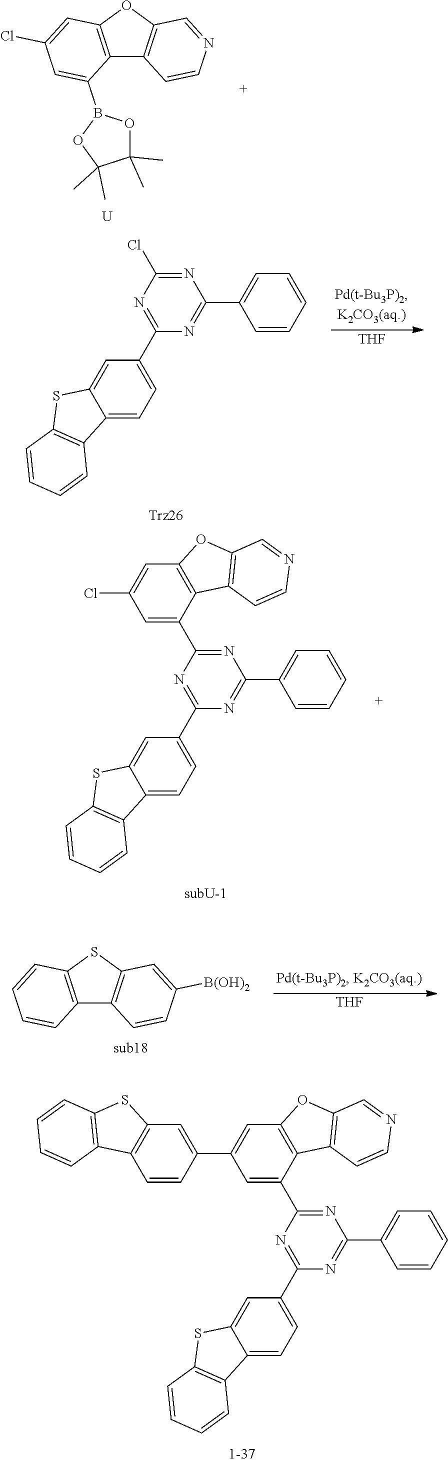

Compound U (15 g, 45.5 mmol) and Compound Trz26 (17.9 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.7 g of Compound subU-1. (Yield: 76%, MS: [M+H]⁺=541)

Compound subU-1 (15 g, 27.7 mmol) and Compound sub18 (6.6 g, 29.1 mmol) were added to 300 ml of THF, and the mixture was stirred and refluxed. Then, potassium carbonate (11.5 g, 83.2 mmol) was dissolved in 100 ml of water and added thereto, and the mixture was sufficiently stirred and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.5 g of Compound 1-37. (Yield: 71%, MS: [M+H]⁺=689)

### Synthesis Example 1-38

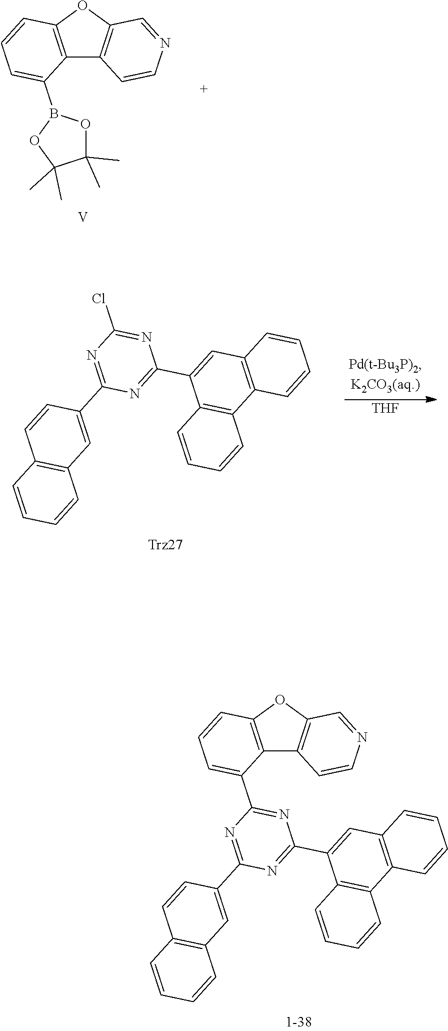

Compound V (15 g, 50.8 mmol) and Compound Trz27 (22.3 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.8 g of Compound 1-38. (Yield: 60%, MS: [M+H]⁺=551)

### Synthesis Example 1-39

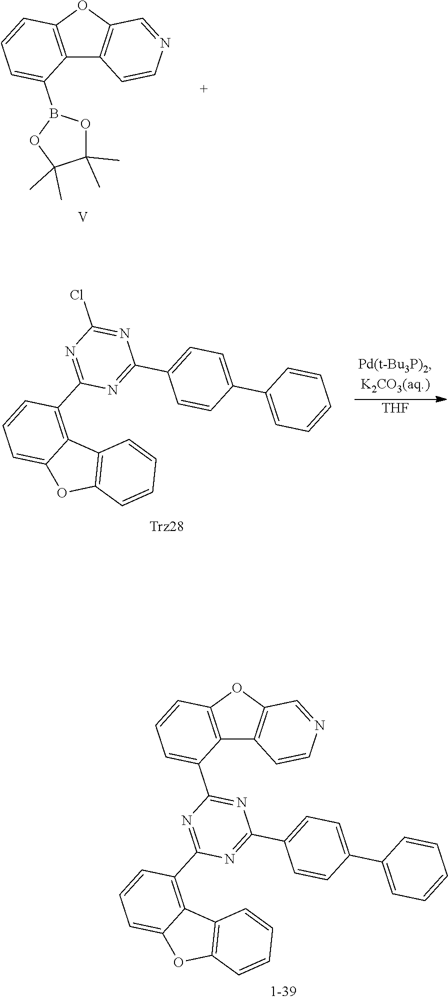

Compound V (15 g, 50.8 mmol) and Compound Trz28 (23.2 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 20.1 g of Compound 1-39. (Yield: 70%, MS: [M+H]⁺=567)

### Synthesis Example 1-40

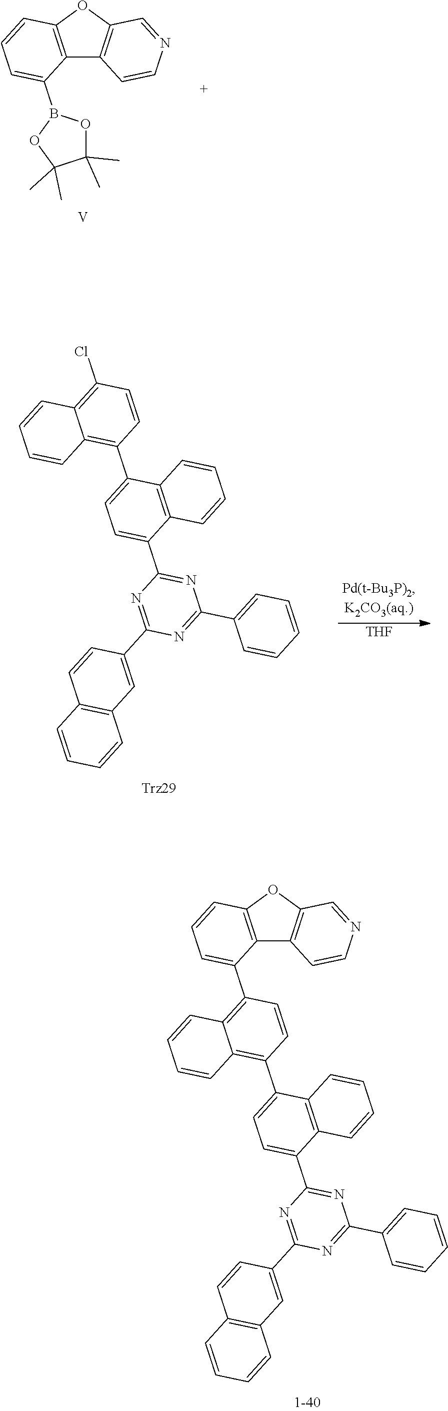

Compound V (15 g, 50.8 mmol) and Compound Trz29 (30.4 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 24.6 g of Compound 1-40. (Yield: 69%, MS: [M+H]⁺=703)

### Synthesis Example 1-41

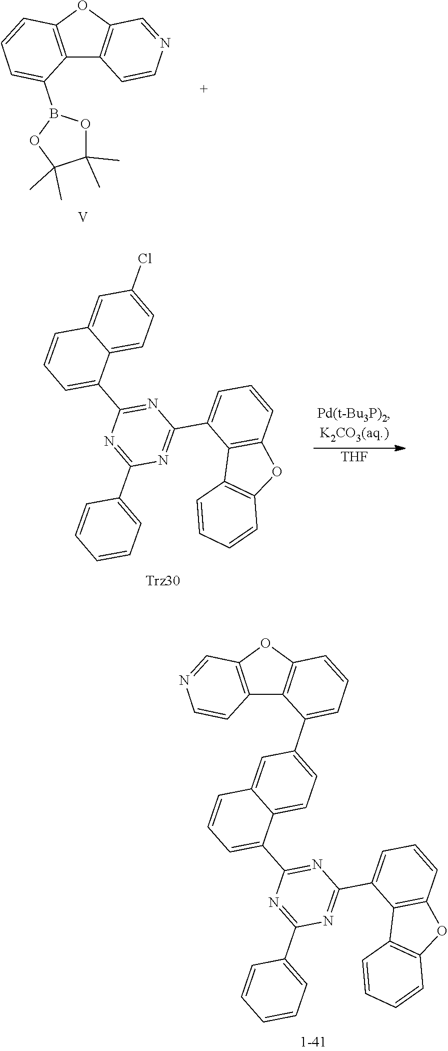

Compound V (15 g, 50.8 mmol) and Compound Trz30 (25.8 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 23.8 g of Compound 1-41. (Yield: 76%, MS: [M+H]⁺=617)

### Synthesis Example 1-42

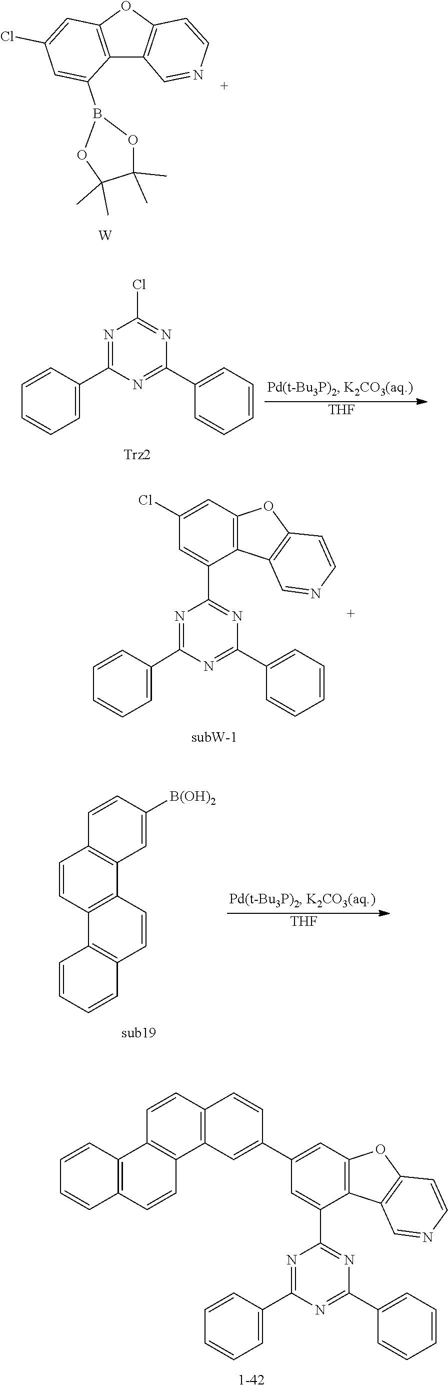

Compound W (15 g, 45.5 mmol) and Compound Trz2 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13 g of Compound subW-1. (Yield: 66%, MS: [M+H]⁺=435)

Compound subW-1 (15 g, 34.5 mmol) and Compound sub19 (9.9 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.4 g of Compound 1-42. (Yield: 76%, MS: [M+H]⁺=627)

### Synthesis Example 1-43

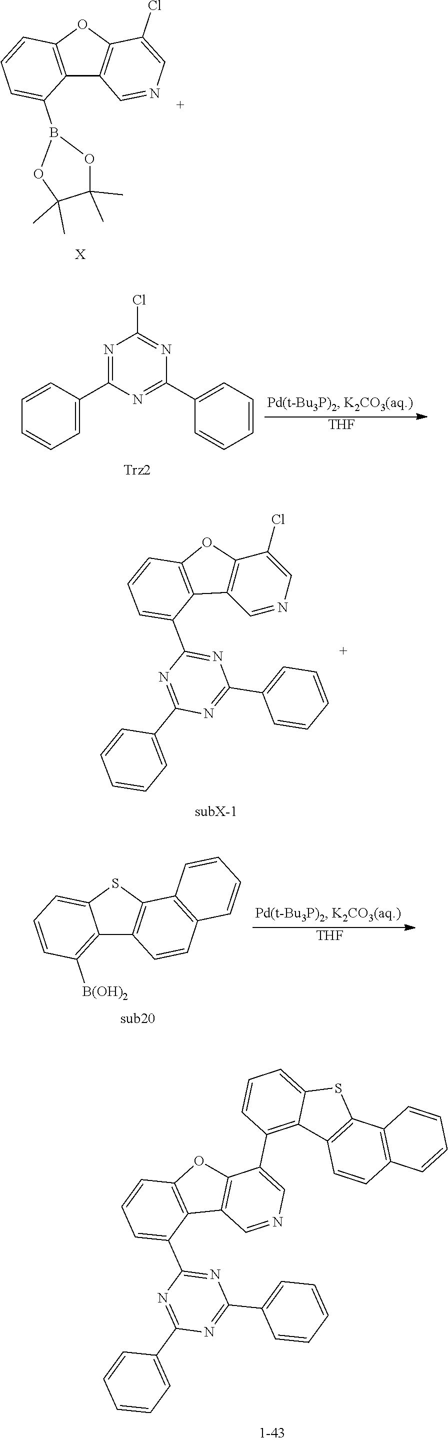

Compound X (15 g, 45.5 mmol) and Compound Trz2 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14 g of Compound subX-1. (Yield: 71%, MS: [M+H]⁺=435)

Compound subX-1 (15 g, 34.5 mmol) and Compound sub20 (10.1 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14 g of Compound 1-43. (Yield: 64%, MS: [M+H]⁺=633)

### Synthesis Example 1-44

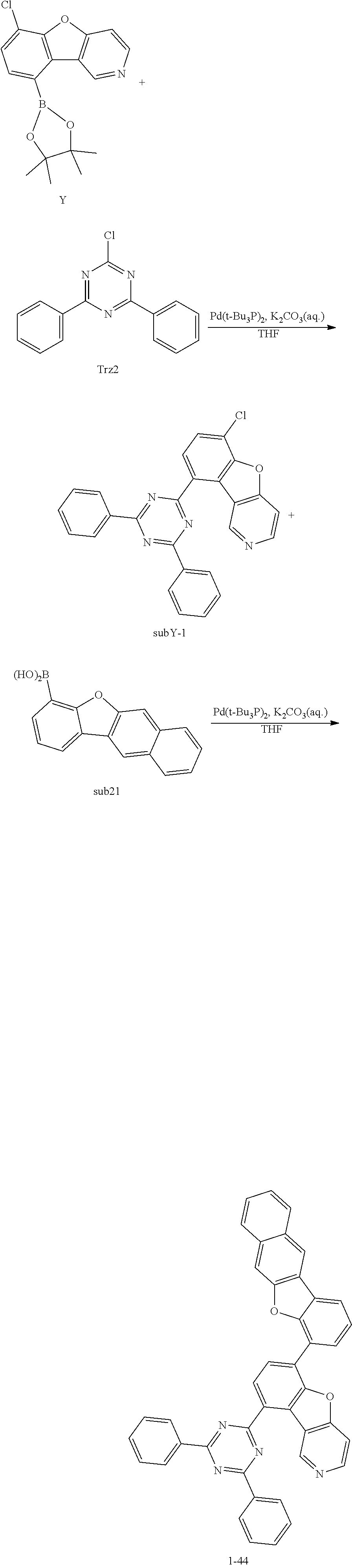

Compound Y (15 g, 45.5 mmol) and Compound Trz2 (12.6 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.8 g of Compound subY-1. (Yield: 80%, MS: [M+H]⁺=435)

Compound subY-1 (15 g, 34.5 mmol) and Compound sub21 (9.5 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.9 g of Compound 1-44. (Yield: 70%, MS: [M+H]⁺=617)

### Synthesis Example 1-45

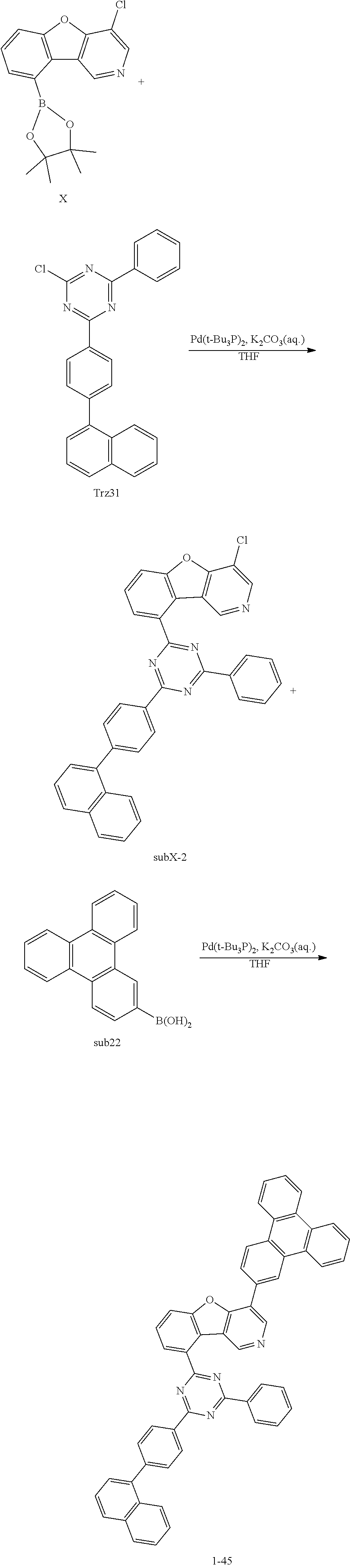

Compound X (15 g, 45.5 mmol) and Compound Trz31 (18.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.1 g of Compound subX-2. (Yield: 71%, MS: [M+H]⁺=561)

Compound subX-2 (15 g, 26.7 mmol) and Compound sub22 (7.6 g, 28.1 mmol) were added to 300 ml of THF, and the mixture was stirred and refluxed. Then, potassium carbonate (11.1 g, 80.2 mmol) was dissolved in 100 ml of water and added thereto, and the mixture was sufficiently stirred and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.7 g of Compound 1-45. (Yield: 78%, MS: [M+H]⁺=753)

### Synthesis Example 1-46

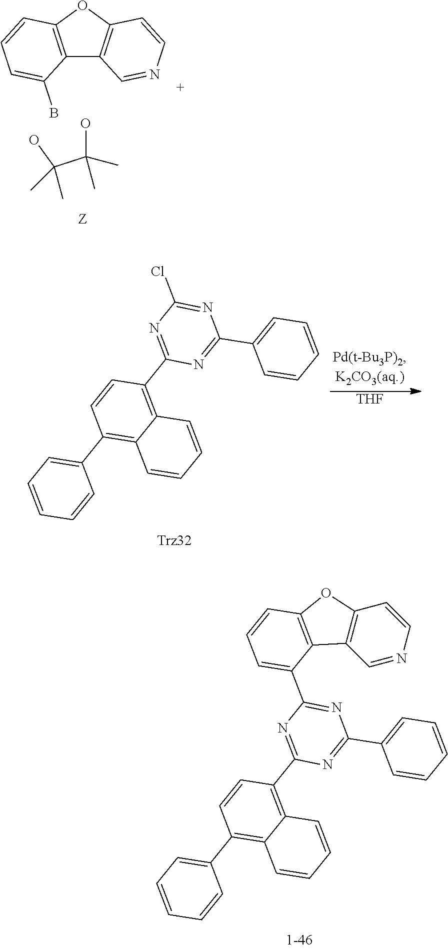

Compound Z (15 g, 50.8 mmol) and Compound Trz32 (21 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.6 g of Compound 1-46. (Yield: 62%, MS: [M+H]⁺=527)

### Synthesis Example 1-47

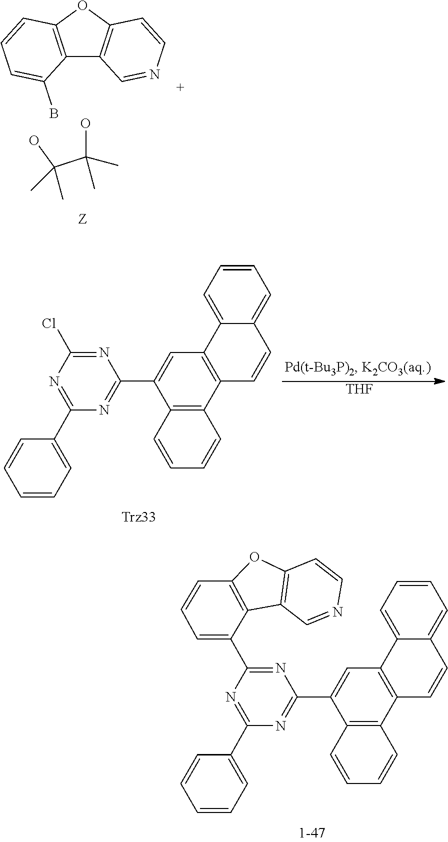

Compound Z (15 g, 50.8 mmol) and Compound Trz33 (22.3 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.3 g of Compound 1-47. (Yield: 69%, MS: [M+H]⁺=551)

### Synthesis Example 1-48

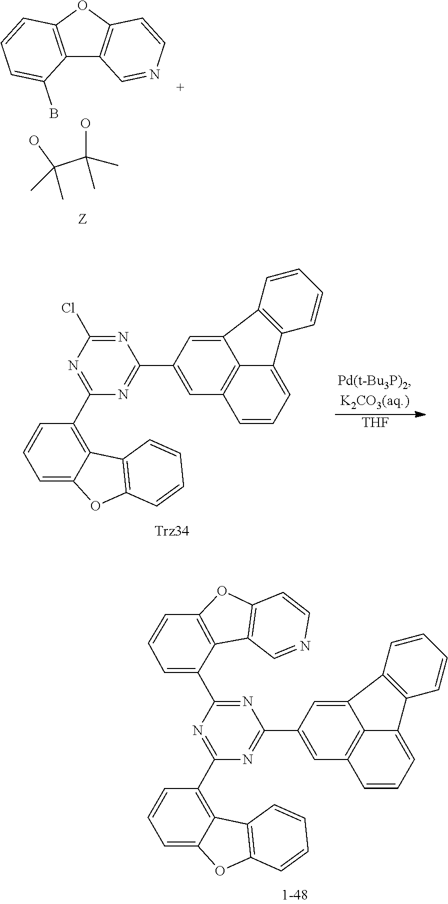

Compound Z (15 g, 50.8 mmol) and Compound Trz34 (25.7 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 23.1 g of Compound 1-48. (Yield: 74%, MS: [M+H]⁺=615)

### Synthesis Example 1-49

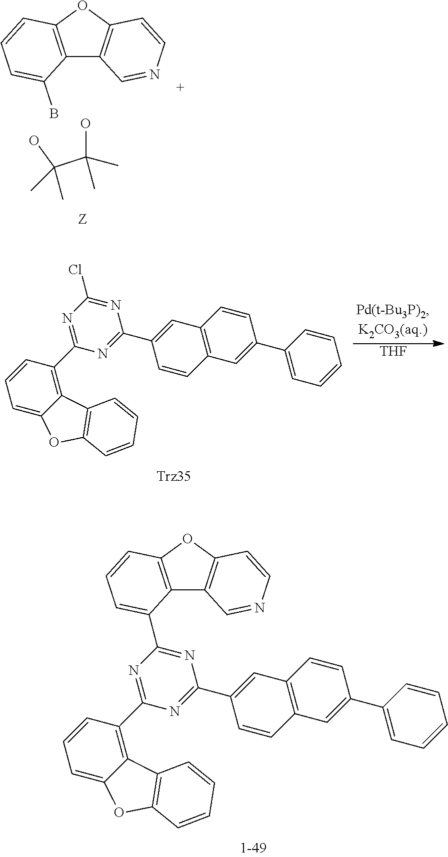

Compound Z (15 g, 50.8 mmol) and Compound Trz35 (25.8 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 22.9 g of Compound 1-49. (Yield: 73%, MS: [M+H]⁺=617)

### Synthesis Example 1-50

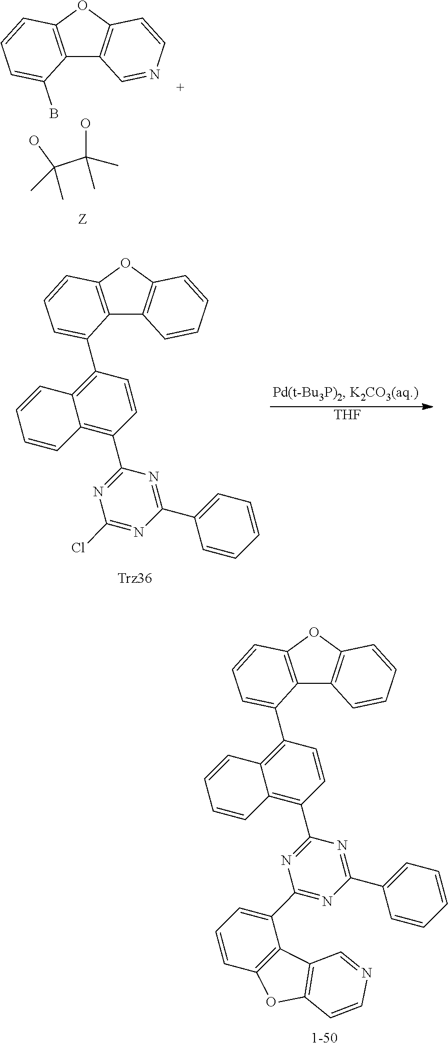

Compound Z (15 g, 50.8 mmol) and Compound Trz36 (25.8 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.4 g of Compound 1-50. (Yield: 62%, MS: [M+H]⁺=617)

### Synthesis Example 1-51

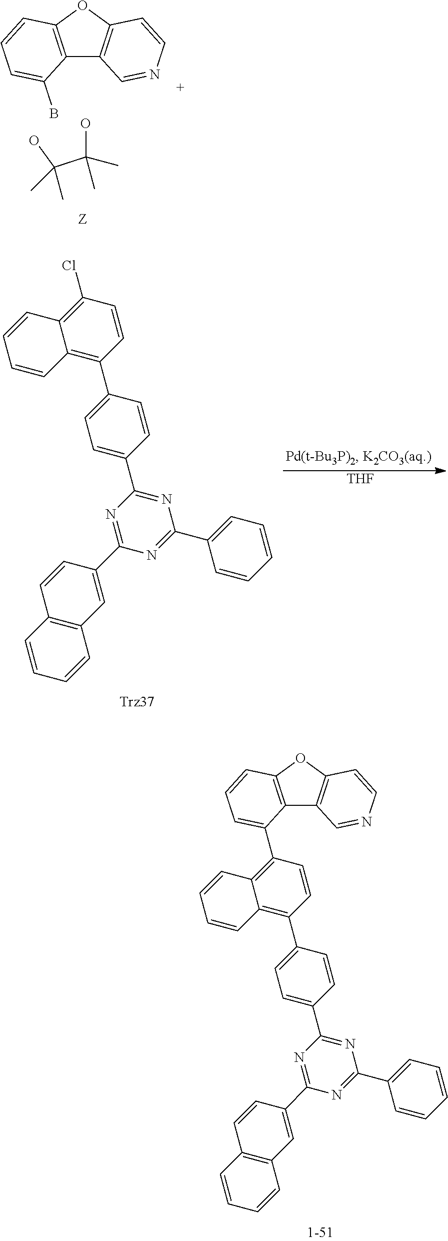

Compound Z (15 g, 50.8 mmol) and Compound Trz37 (27.8 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.9 g of Compound 1-51. (Yield: 60%, MS: [M+H]⁺=653)

### Synthesis Example 1-52

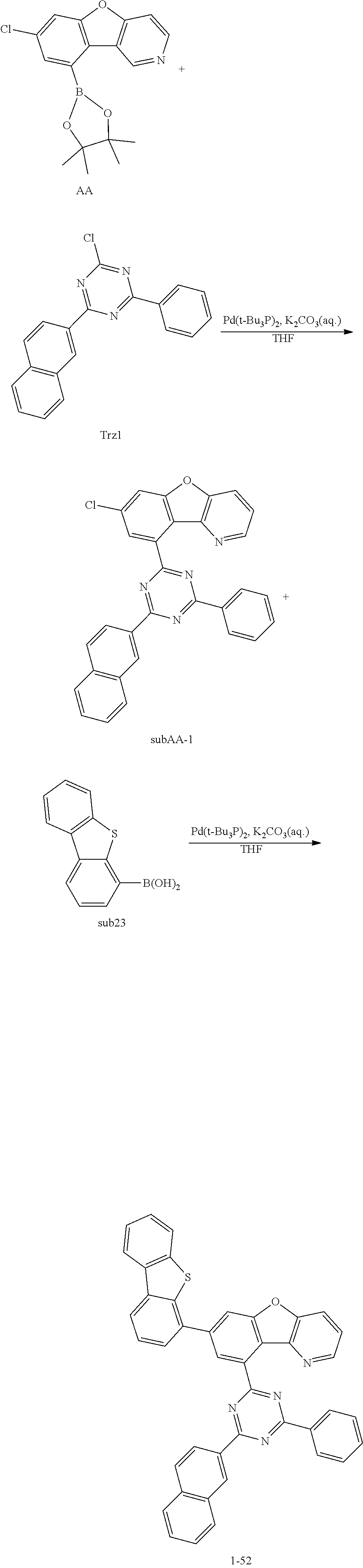

Compound AA (15 g, 45.5 mmol) and Compound Trz1 (15.2 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 17.2 g of Compound subAA-1. (Yield: 78%, MS: [M+H]⁺=485)

Compound subAA-1 (15 g, 30.9 mmol) and Compound sub23 (7.4 g, 32.5 mmol) were added to 300 ml of THF, and the mixture was stirred and refluxed. Then, potassium carbonate (12.8 g, 92.8 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.9 g of Compound 1-52. (Yield: 71%, MS: [M+H]⁺=633)

### Synthesis Example 1-53

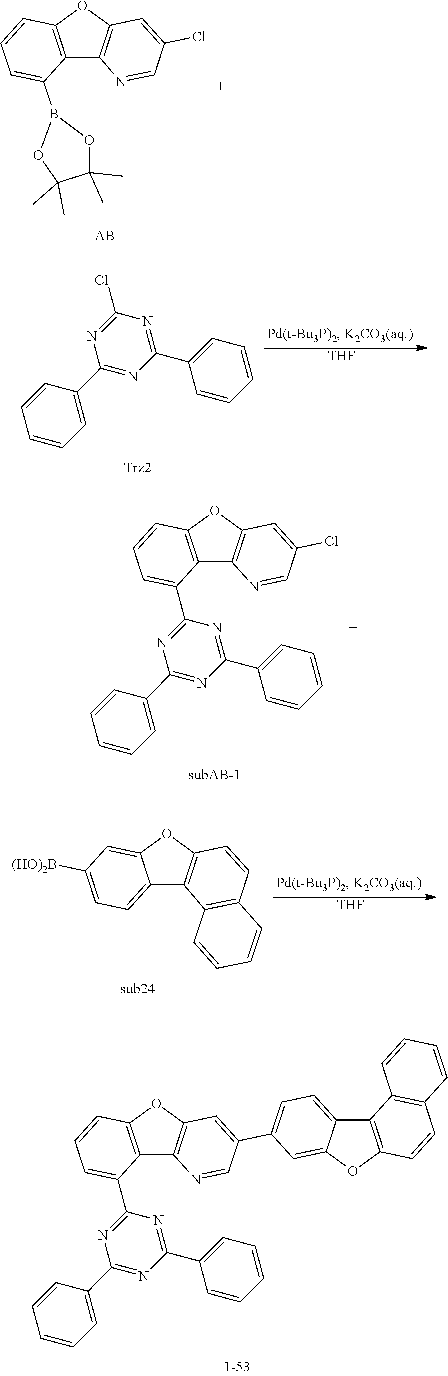

Compound AB (15 g, 45.5 mmol) and Compound Trz2 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14 g of Compound subAB-1. (Yield: 71%, MS: [M+H]⁺=435)

Compound subAB-1 (14 g, 32 mmol) and Compound sub24 (8.9 g, 33.8 mmol) were added to 300 ml of THF, and the mixture was stirred and refluxed. Then, potassium carbonate (13.3 g, 96.6 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.5 g of Compound 1-53. (Yield: 62%, MS: [M+H]⁺=617)

### Synthesis Example 1-54

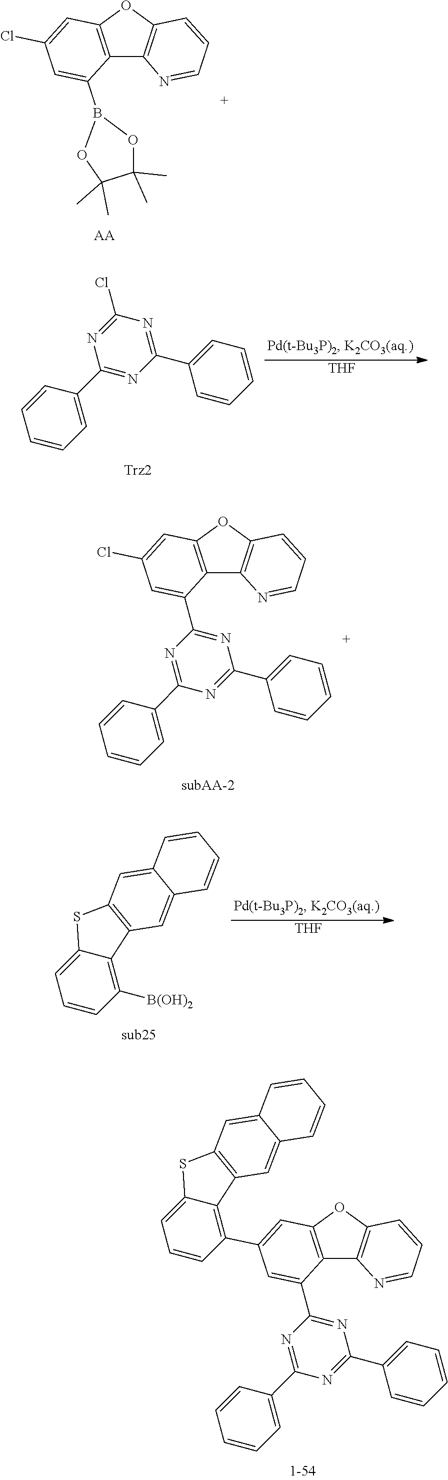

Compound AA (15 g, 45.5 mmol) and Compound Trz2 (12.8 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.6 g of Compound subAA-2. (Yield: 64%, MS: [M+H]⁺=435)

Compound subAA-2 (15 g, 34.5 mmol) and Compound sub25 (10.1 g, 36.2 mmol) were added to 300 ml of THF, and the mixture was stirred and refluxed. Then, potassium carbonate (14.3 g, 103.5 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.3 g of Compound 1-54. (Yield: 61%, MS: [M+H]⁺=633)

### Synthesis Example 1-55

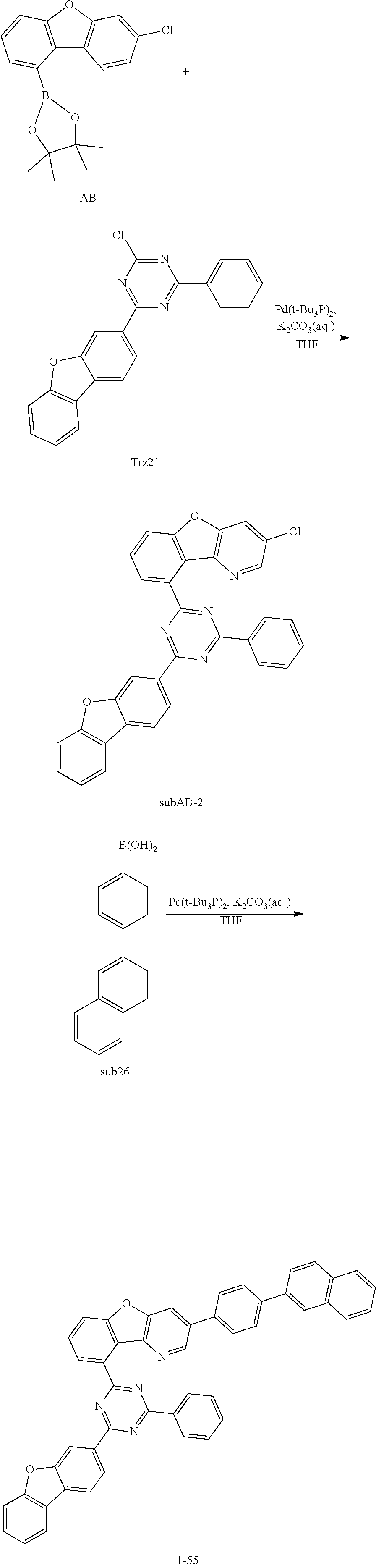

Compound AB (15 g, 45.5 mmol) and Compound Trz21 (17.1 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.5 g of Compound subAB-2. (Yield: 65%, MS: [M+H]⁺=525)

Compound subAB-2 (15 g, 28.6 mmol) and Compound sub26 (7.4 g, 30 mmol) were added to 300 ml of THF, and the mixture was stirred and refluxed. Then, potassium carbonate (11.8 g, 85.7 mmol) was dissolved in 100 ml of water and added thereto, and the mixture was sufficiently stirred and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.5 g of Compound 1-55. (Yield: 63%, MS: [M+H]⁺=693)

### Synthesis Example 1-56

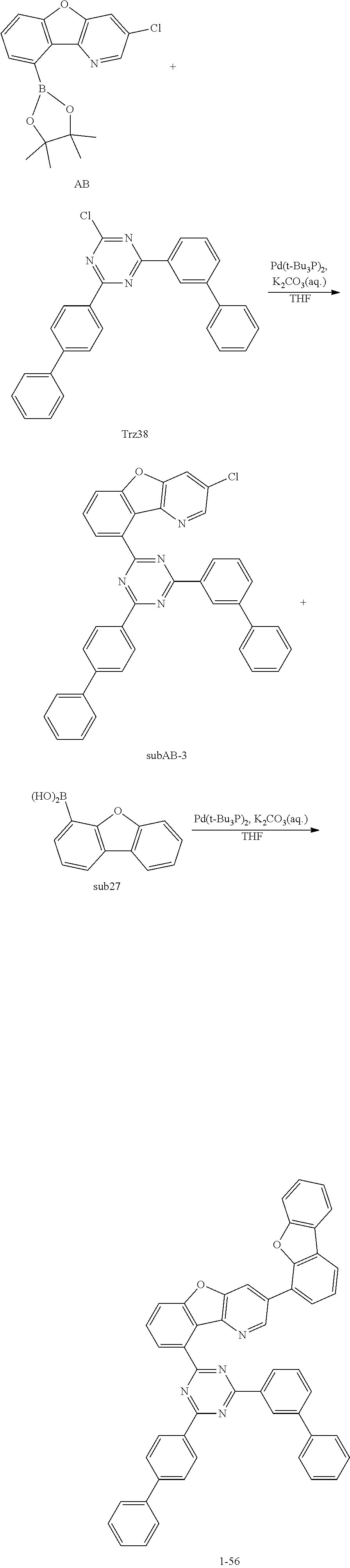

Compound AB (15 g, 45.5 mmol) and Compound Trz38 (20.1 g, 47.8 mmol) were added to 300 ml of THF, and the mixture was stirred and refluxed. Then, potassium carbonate (18.9 g, 136.5 mmol) was dissolved in 100 ml of water and added thereto, and the mixture was sufficiently stirred and then bis(tri-tert-butylphosphine)palladium(0) (0.2 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.4 g of Compound subAB-3. (Yield: 69%, MS: [M+H]⁺=587)

Compound subAB-3 (15 g, 25.6 mmol) and Compound sub27 (5.7 g, 26.8 mmol) were added to 300 ml of THF, and the mixture was stirred and refluxed. Then, potassium carbonate (10.6 g, 76.7 mmol) was dissolved in 100 ml of water and added thereto, and the mixture was sufficiently stirred and then bis(tri-tert-butylphosphine)palladium(0) (0.1 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.4 g of Compound 1-56. (Yield: 73%, MS: [M+H]⁺=719)

### Synthesis Example 1-57

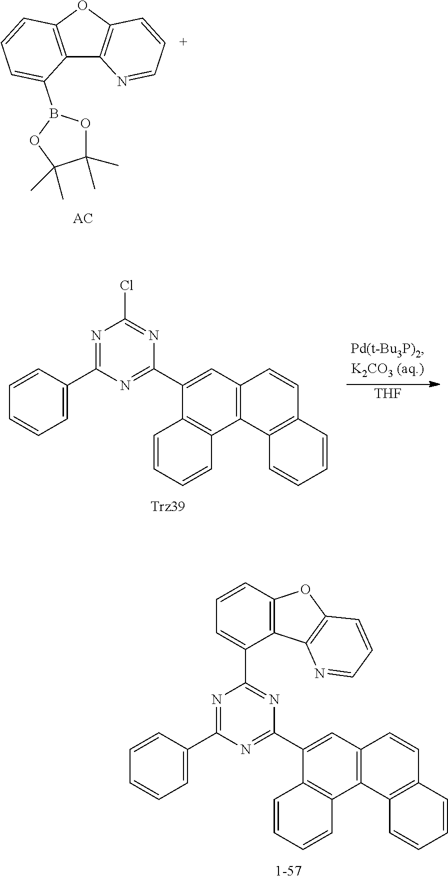 [SMILES: CC1(C)OB(c2cccc3oc4cccnc4c23)OC1(C)C ; Clc1nc(-c2ccccc2)nc(-c2cc3ccc4ccccc4c3c3ccccc23)n1 ; c1ccc(-c2nc(-c3cc4ccc5ccccc5c4c4ccccc34)nc(-c3cccc4oc5cccnc5c34)n2)cc1 ; "THF"]

Compound AC (15 g, 50.8 mmol) and Compound Trz39 (22.3 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 22.1 g of Compound 1-57. (Yield: 79%, MS: [M+H]⁺=551)

### Synthesis Example 1-58

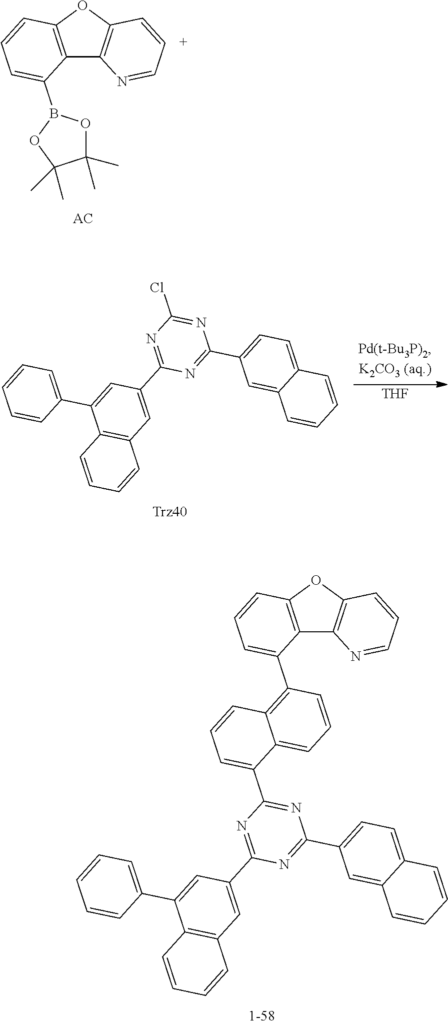 [SMILES: CC1(C)OB(c2cccc3oc4cccnc4c23)OC1(C)C ; Clc1nc(-c2ccc3ccccc3c2)nc(-c2cc(-c3ccccc3)c3ccccc3c2)n1 ; c1ccc(-c2cc(-c3nc(-c4ccc5ccccc5c4)nc(-c4cccc5c(-c6cccc7oc8cccnc8c67)cccc45)n3)cc3ccccc23)cc1 ; "THF"]

Compound AC (15 g, 50.8 mmol) and Compound Trz40 (23.7 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 19.3 g of Compound 1-58. (Yield: 66%, MS: [M+H]⁺=577)

### Synthesis Example 1-59

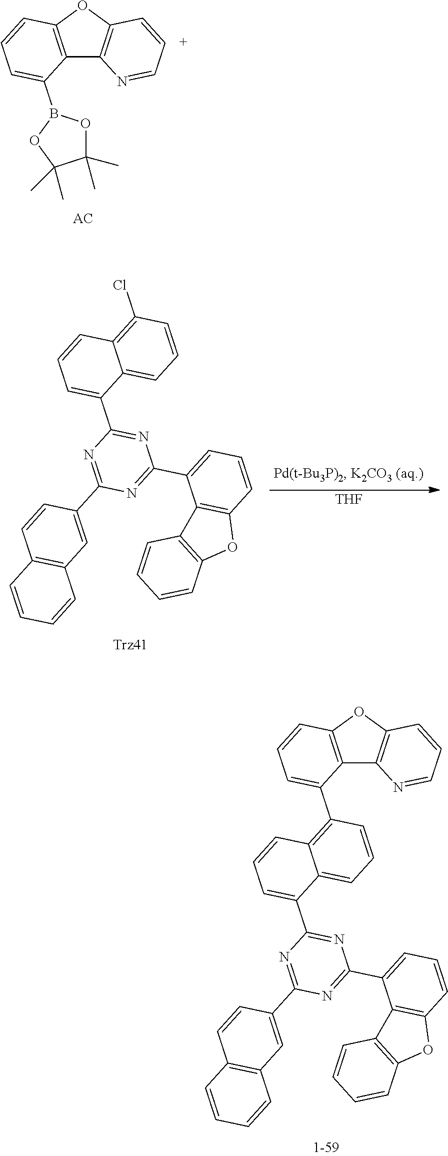 [SMILES: CC1(C)OB(c2cccc3oc4cccnc4c23)OC1(C)C ; Clc1cccc2c(-c3nc(-c4ccc5ccccc5c4)nc(-c4cccc5oc6ccccc6c45)n3)cccc12 ; c1ccc2cc(-c3nc(-c4cccc5c(-c6cccc7oc8cccnc8c67)cccc45)nc(-c4cccc5oc6ccccc6c45)n3)ccc2c1 ; "THF"]

Compound AC (15 g, 50.8 mmol) and Compound Trz41 (28.5 g, 53.4 mmol) were added to 300 ml of THF, and the mixture was stirred and refluxed. Then, potassium carbonate (21.1 g, 152.5 mmol) was dissolved in 100 ml of water and added thereto, and the mixture was sufficiently stirred and then bis(tri-tert-butylphosphine)palladium(0) (0.3 g, 0.5 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 24.7 g of Compound 1-59. (Yield: 73%, MS: [M+H]⁺=667)

### Synthesis Example 2-1

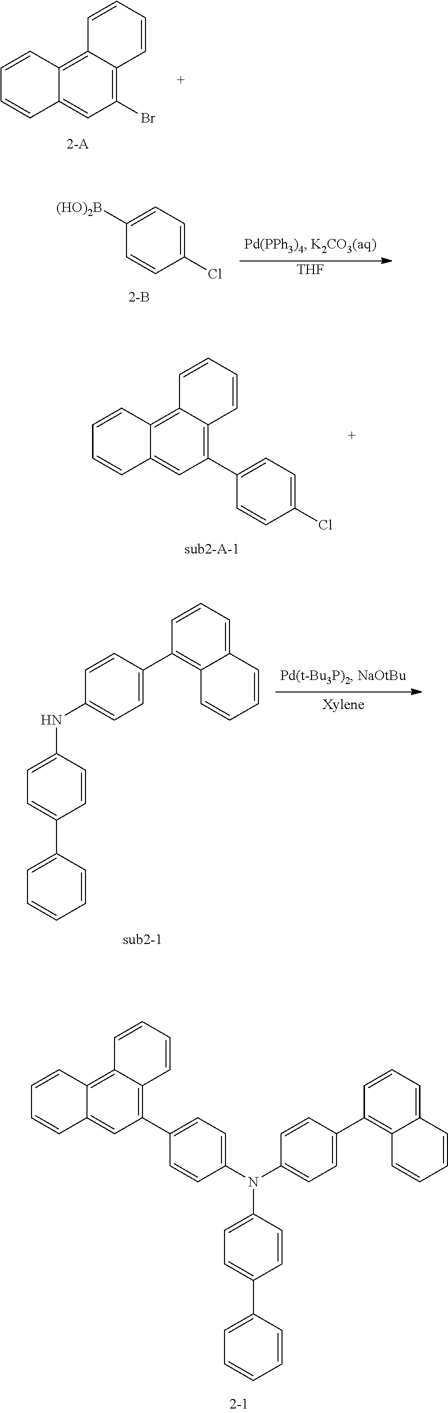

Compound 2-A (15 g, 58.3 mmol) and Compound 2-B (10 g, 64.2 mmol) were added to 300 ml of THE under a nitrogen atmosphere, and the mixture was stirred and refluxed. Then, potassium carbonate (16.1 g, 116.7 mmol) was dissolved in 48 ml of water and added thereto, and the mixture was sufficiently stirred and then tetrakis(triphenylphosphine)palladium(0) (1.3 g, 1.2 mmol) was added. After reacting for 11 hours, the reaction mixture was cooled to room temperature, then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.6 g of Compound sub2-A-1. (Yield: 75%, MS: [M+H]⁺=289)

Compound sub2-A-1 (10 g, 34.6 mmol), Compound sub2-1 (12.9 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.7 g of Compound 2-1. (Yield: 59%, MS: [M+H]⁺=624)

### Synthesis Example 2-2

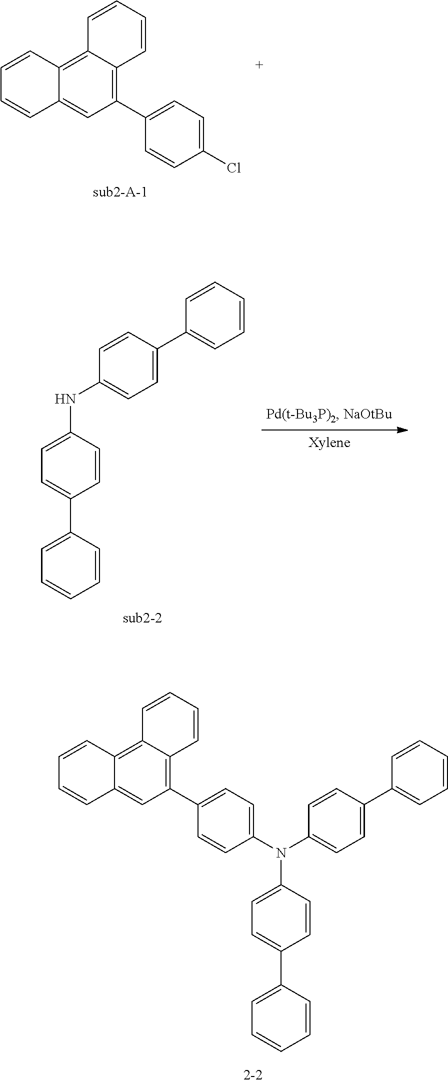

Compound sub2-A-1 (10 g, 34.6 mmol), Compound sub2-2 (11.1 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 10.1 g of Compound 2-2. (Yield: 51%, MS: [M+H]⁺=574)

### Synthesis Example 2-3

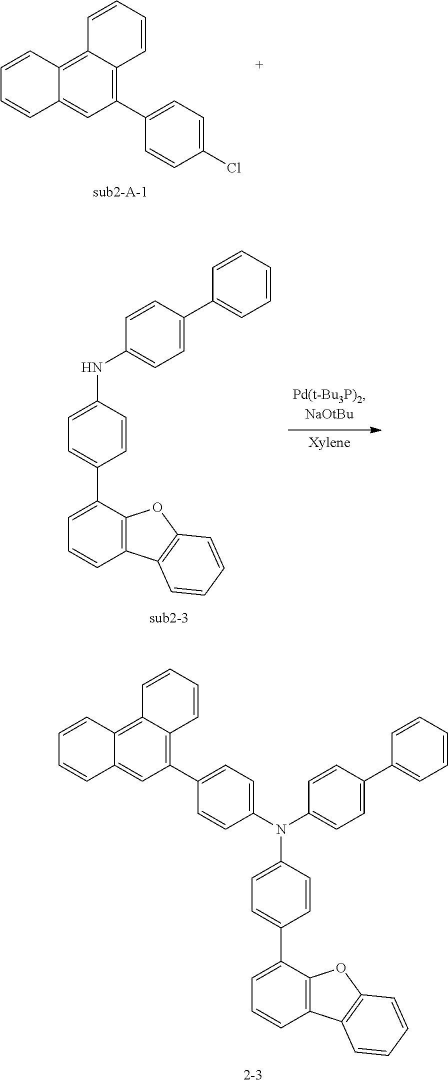

Compound sub2-A-1 (10 g, 34.6 mmol), Compound sub2-3 (14.3 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.2 g of Compound 2-3. (Yield: 53%, MS: [M+H]⁺=664)

### Synthesis Example 2-4

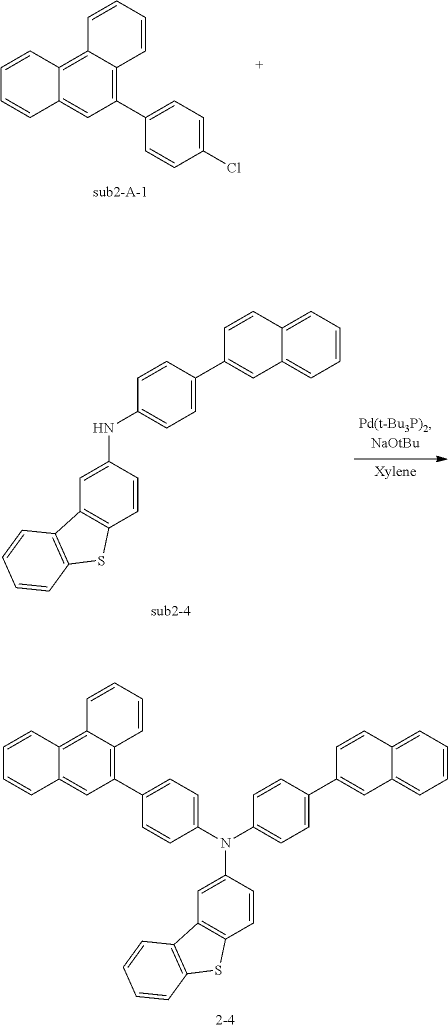 [SMILES: Clc1ccc(-c2cc3ccccc3c3ccccc23)cc1 ; c1ccc2cc(-c3ccc(Nc4ccc5sc6ccccc6c5c4)cc3)ccc2c1 ; c1ccc2cc(-c3ccc(N(c4ccc(-c5cc6ccccc6c6ccccc56)cc4)c4ccc5sc6ccccc6c5c4)cc3)ccc2c1]

Compound sub2-A-1 (10 g, 34.6 mmol), Compound sub2-4 (13.9 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14 g of Compound 2-4. (Yield: 62%, MS: [M+H]⁺=654)

### Synthesis Example 2-5

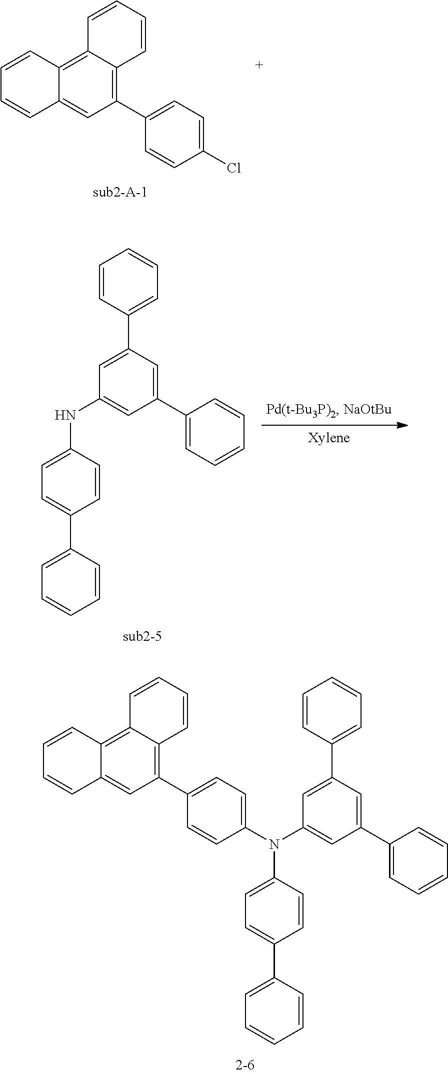 [SMILES: Clc1ccc(-c2cc3ccccc3c3ccccc23)cc1 ; c1ccc(-c2ccc(Nc3cc(-c4ccccc4)cc(-c4ccccc4)c3)cc2)cc1 ; c1ccc(-c2ccc(N(c3ccc(-c4cc5ccccc5c5ccccc45)cc3)c3cc(-c4ccccc4)cc(-c4ccccc4)c3)cc2)cc1]

Compound sub2-A-1 (10 g, 34.6 mmol), Compound sub2-5 (13.8 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.2 g of Compound 2-5. (Yield: 50%, MS: [M+H]⁺=650)

### Synthesis Example 2-6

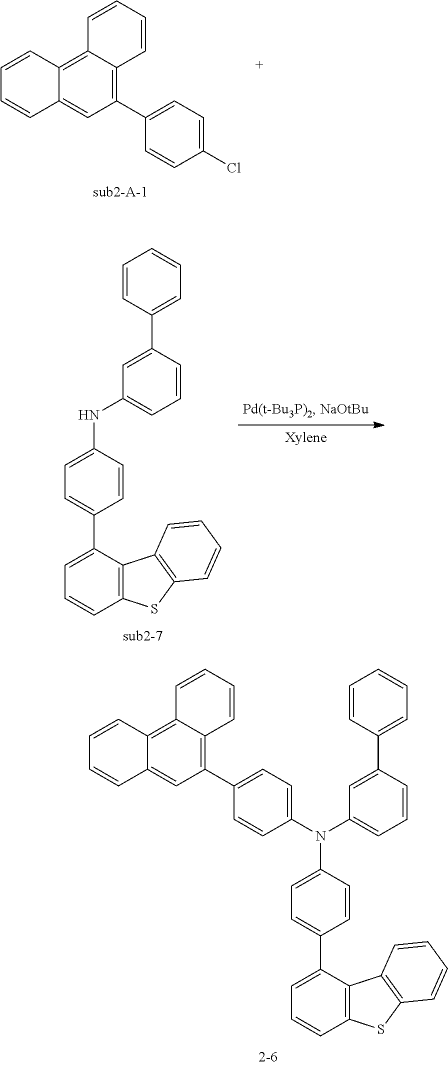 [SMILES: Clc1ccc(-c2cc3ccccc3c3ccccc23)cc1 ; c1ccc(-c2cccc(Nc3ccc(-c4cccc5sc6ccccc6c45)cc3)c2)cc1 ; c1ccc(-c2cccc(N(c3ccc(-c4cc5ccccc5c5ccccc45)cc3)c3ccc(-c4cccc5sc6ccccc6c45)cc3)c2)cc1]

Compound sub2-A-1 (10 g, 34.6 mmol), Compound sub2-6 (14.8 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.2 g of Compound 2-6. (Yield: 52%, MS: [M+H]⁺=680)

### Synthesis Example 2-7

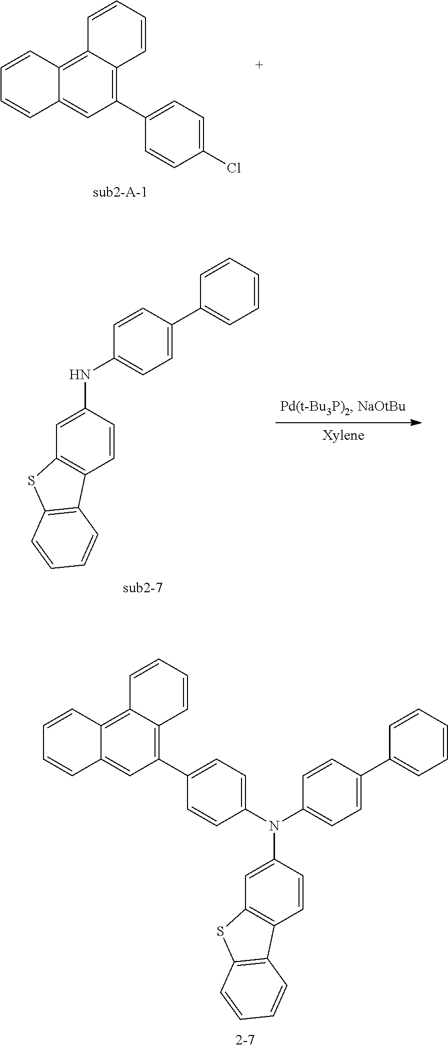 [SMILES: Clc1ccc(-c2cc3ccccc3c3ccccc23)cc1 ; c1ccc(-c2ccc(Nc3ccc4c(c3)sc3ccccc34)cc2)cc1 ; c1ccc(-c2ccc(N(c3ccc(-c4cc5ccccc5c5ccccc45)cc3)c3ccc4c(c3)sc3ccccc34)cc2)cc1]

Compound sub2-A-1 (10 g, 34.6 mmol), Compound sub2-7 (12.2 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 1 g of Compound 2-7. (Yield: 50%, MS: [M+H]⁺=61)

### Synthesis Example 2-8

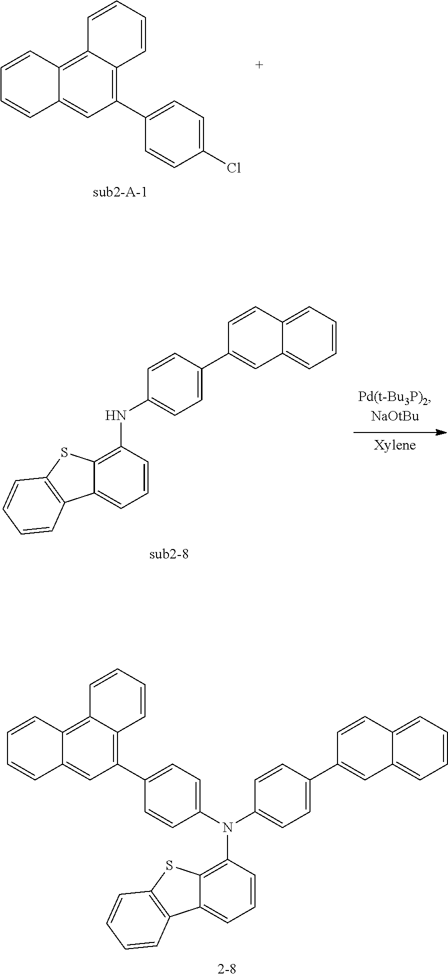 [SMILES: Clc1ccc(-c2cc3ccccc3c3ccccc23)cc1 ; c1ccc2cc(-c3ccc(Nc4cccc5c4sc4ccccc45)cc3)ccc2c1 ; c1ccc2cc(-c3ccc(N(c4ccc(-c5cc6ccccc6c6ccccc56)cc4)c4cccc5c4sc4ccccc45)cc3)ccc2c1]

Compound sub2-A-1 (10 g, 34.6 mmol), Compound sub2-8 (13.9 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.3 g of Compound 2-8. (Yield: 59%, MS: [M+H]⁺=654)

### Synthesis Example 2-9

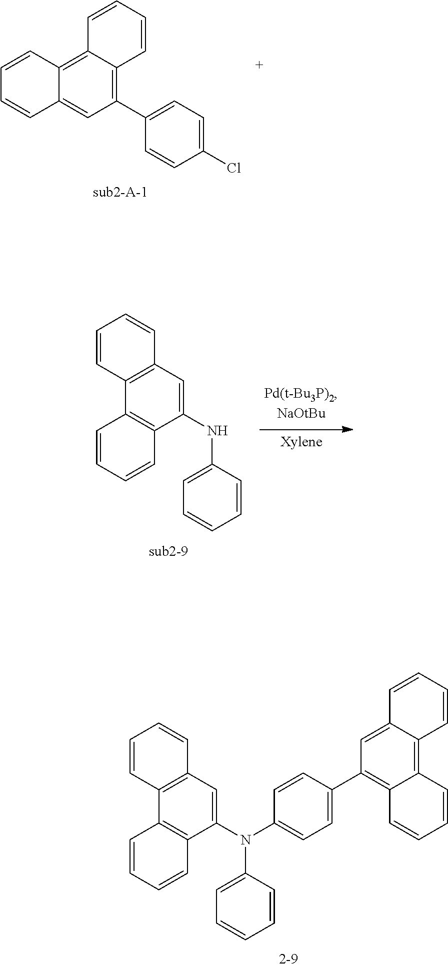 [SMILES: Clc1ccc(-c2cc3ccccc3c3ccccc23)cc1 ; c1ccc(Nc2cc3ccccc3c3ccccc23)cc1 ; c1ccc(N(c2ccc(-c3cc4ccccc4c4ccccc34)cc2)c2cc3ccccc3c3ccccc23)cc1]

Compound sub2-A-1 (10 g, 34.6 mmol), Compound sub2-9 (9.3 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.2 g of Compound 2-9. (Yield: 62%, MS: [M+H]⁺=522)

### Synthesis Example 2-10

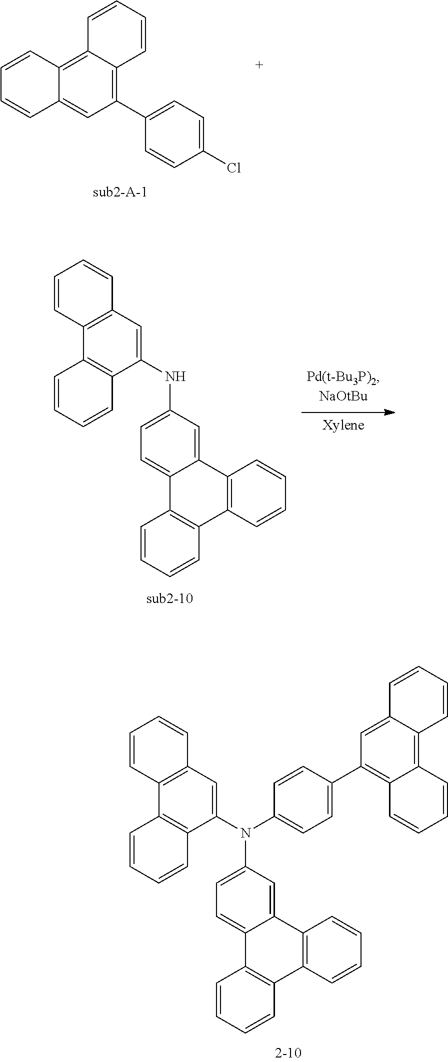 [SMILES: Clc1ccc(-c2cc3ccccc3c3ccccc23)cc1 ; c1ccc2c(c1)cc(Nc1ccc3c4ccccc4c4ccccc4c3c1)c1ccccc12 ; c1ccc2c(c1)cc(-c1ccc(N(c3ccc4c5ccccc5c5ccccc5c4c3)c3cc4ccccc4c4ccccc34)cc1)c1ccccc12]

Compound sub2-A-1 (10 g, 34.6 mmol), Compound sub2-10 (14.5 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14.4 g of Compound 2-10. (Yield: 62%, MS: [M+H]⁺=672)

### Synthesis Example 2-11

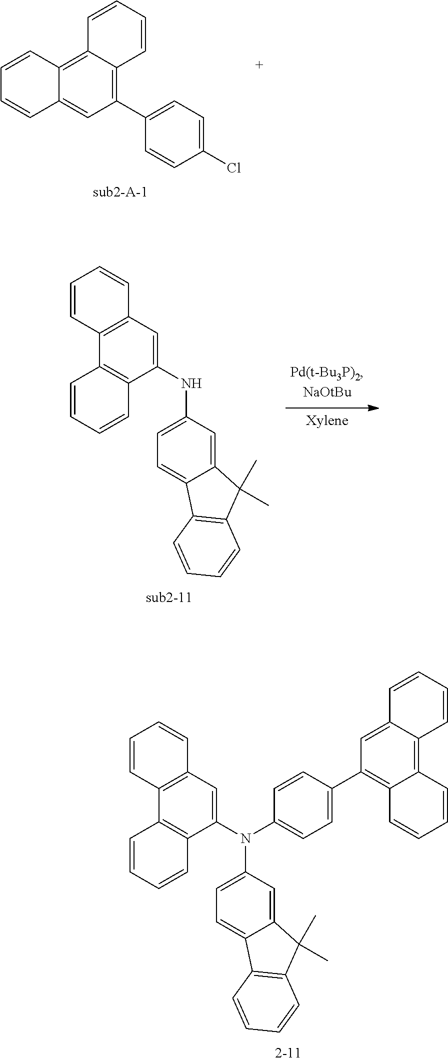 [SMILES: Clc1ccc(-c2cc3ccccc3c3ccccc23)cc1 ; CC1(C)c2ccccc2-c2ccc(Nc3cc4ccccc4c4ccccc34)cc21 ; CC1(C)c2ccccc2-c2ccc(N(c3ccc(-c4cc5ccccc5c5ccccc45)cc3)c3cc4ccccc4c4ccccc34)cc21]

Compound sub2-A-1 (10 g, 34.6 mmol), Compound sub2-11 (13.4 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.4 g of Compound 2-11. (Yield: 56%, MS: [M+H]⁺=638)

### Synthesis Example 2-12

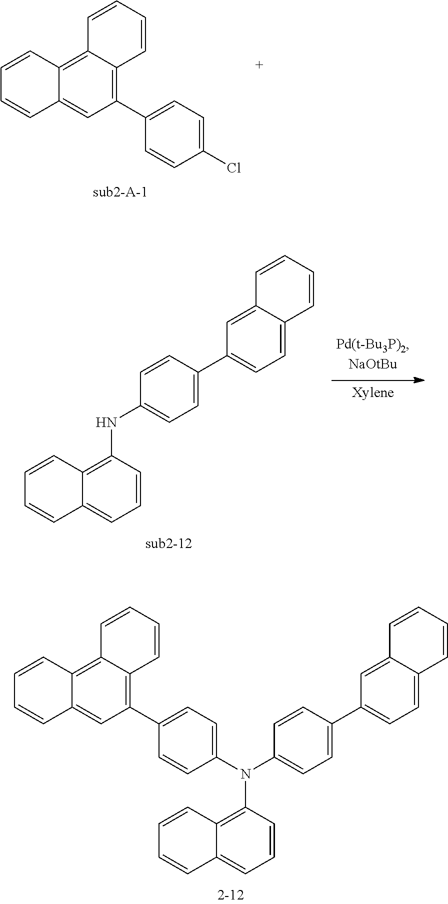 [SMILES: Clc1ccc(-c2cc3ccccc3c3ccccc23)cc1 ; c1ccc2cc(-c3ccc(Nc4cccc5ccccc45)cc3)ccc2c1 ; c1ccc2cc(-c3ccc(N(c4ccc(-c5cc6ccccc6c6ccccc56)cc4)c4cccc5ccccc45)cc3)ccc2c1]

Compound sub2-A-1 (10 g, 34.6 mmol), Compound sub2-12 (12 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11 g of Compound 2-12. (Yield: 53%, MS: [M+H]⁺=598)

### Synthesis Example 2-13

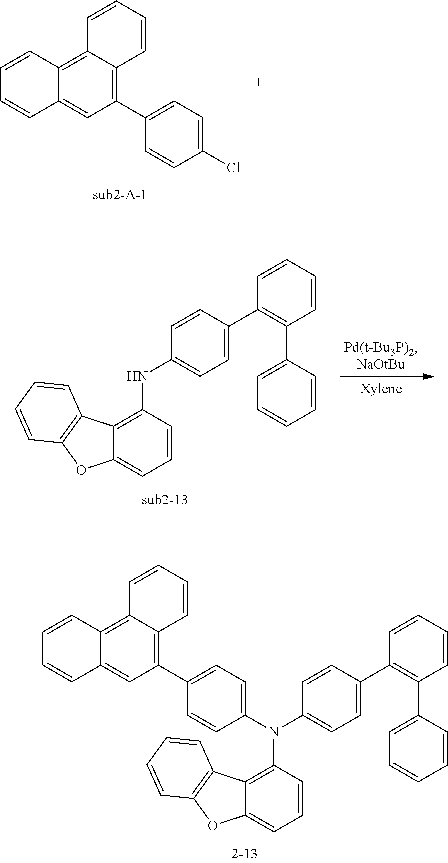 [SMILES: Clc1ccc(-c2cc3ccccc3c3ccccc23)cc1 ; c1ccc(-c2ccccc2-c2ccc(Nc3cccc4oc5ccccc5c34)cc2)cc1 ; c1ccc(-c2ccccc2-c2ccc(N(c3ccc(-c4cc5ccccc5c5ccccc45)cc3)c3cccc4oc5ccccc5c34)cc2)cc1]

Compound sub2-A-1 (10 g, 34.6 mmol), Compound sub2-13 (14.3 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 15.6 g of Compound 2-13. (Yield: 68%, MS: [M+H]⁺=664)

### Synthesis Example 2-14

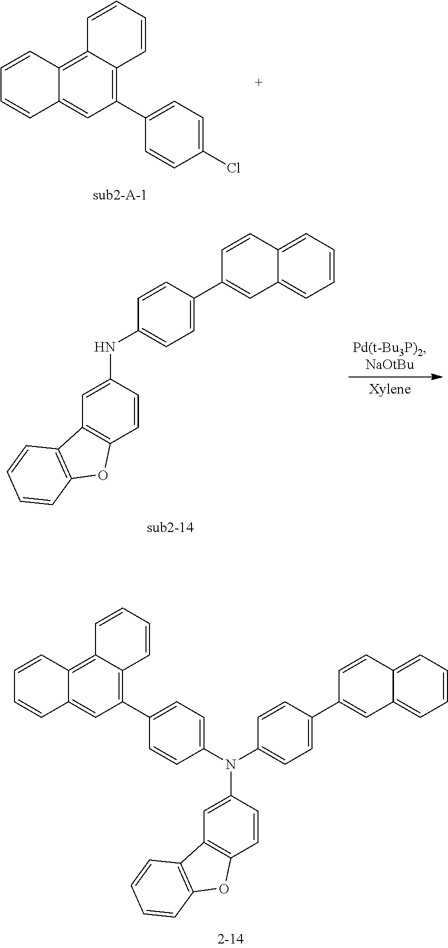 [SMILES: Clc1ccc(-c2cc3ccccc3c3ccccc23)cc1 ; c1ccc2cc(-c3ccc(Nc4ccc5oc6ccccc6c5c4)cc3)ccc2c1 ; c1ccc2cc(-c3ccc(N(c4ccc(-c5cc6ccccc6c6ccccc56)cc4)c4ccc5oc6ccccc6c5c4)cc3)ccc2c1]

Compound sub2-A-1 (10 g, 34.6 mmol), Compound sub2-14 (13.3 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.2 g of Compound 2-14. (Yield: 60%, MS: [M+H]⁺=638)

### Synthesis Example 2-15

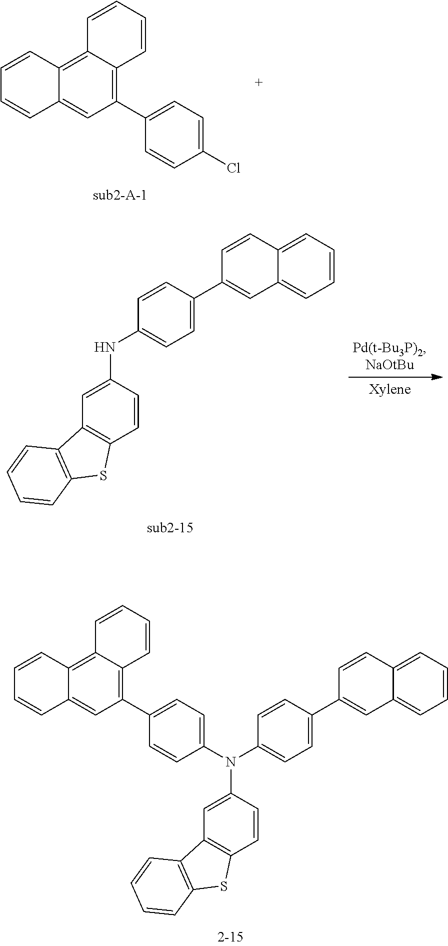 [SMILES: Clc1ccc(-c2cc3ccccc3c3ccccc23)cc1 ; c1ccc2cc(-c3ccc(Nc4ccc5sc6ccccc6c5c4)cc3)ccc2c1 ; c1ccc2cc(-c3ccc(N(c4ccc(-c5cc6ccccc6c6ccccc56)cc4)c4ccc5sc6ccccc6c5c4)cc3)ccc2c1]

Compound sub2-A-1 (10 g, 34.6 mmol), Compound sub2-15 (13.9 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12 g of Compound 2-15. (Yield: 53%, MS: [M+H]⁺=654)

### Synthesis Example 2-16

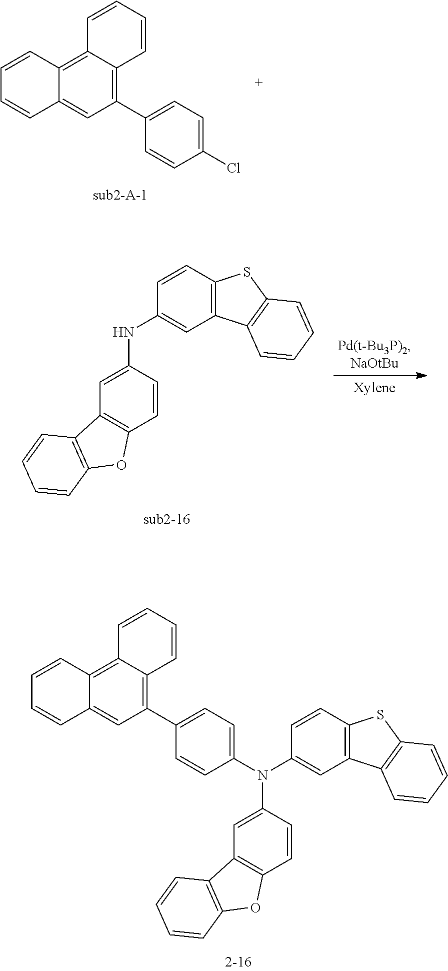 [SMILES: Clc1ccc(-c2cc3ccccc3c3ccccc23)cc1 ; c1ccc2c(c1)oc1ccc(Nc3ccc4sc5ccccc5c4c3)cc12 ; c1ccc2c(c1)cc(-c1ccc(N(c3ccc4oc5ccccc5c4c3)c3ccc4sc5ccccc5c4c3)cc1)c1ccccc12]

Compound sub2-A-1 (10 g, 34.6 mmol), Compound sub2-16 (12.7 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.7 g of Compound 2-16. (Yield: 64%, MS: [M+H]⁺=618)

### Synthesis Example 2-17

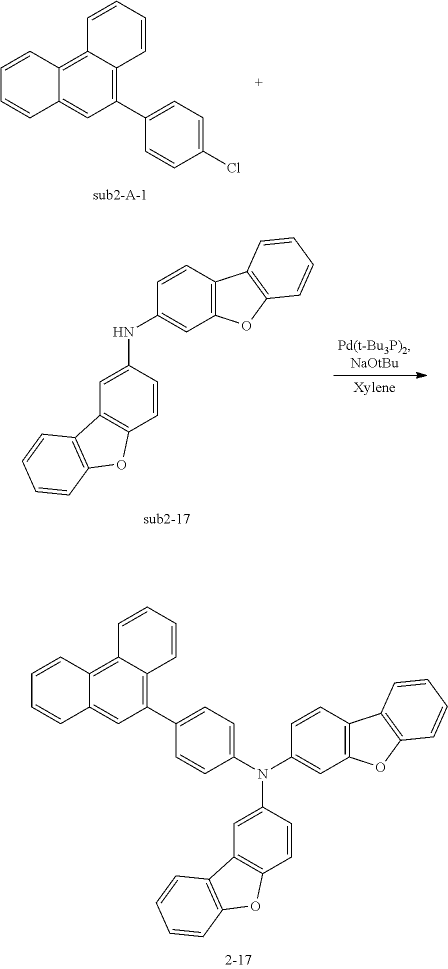 [SMILES: Clc1ccc(-c2cc3ccccc3c3ccccc23)cc1 ; c1ccc2c(c1)oc1cc(Nc3ccc4oc5ccccc5c4c3)ccc12 ; c1ccc2c(c1)cc(-c1ccc(N(c3ccc4c(c3)oc3ccccc34)c3ccc4oc5ccccc5c4c3)cc1)c1ccccc12]

Compound sub2-A-1 (10 g, 34.6 mmol), Compound sub2-17 (12.1 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.5 g of Compound 2-17. (Yield: 55%, MS: [M+H]⁺=602)

### Synthesis Example 2-18

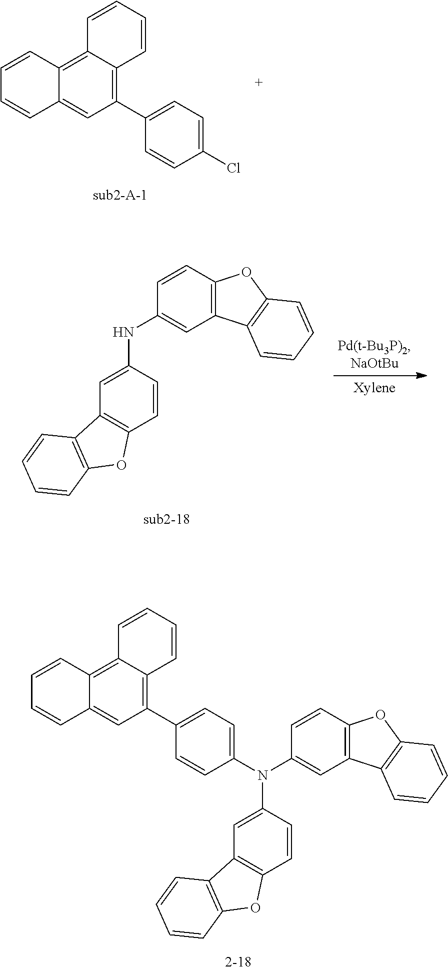 [SMILES: Clc1ccc(-c2cc3ccccc3c3ccccc23)cc1 ; c1ccc2c(c1)oc1ccc(Nc3ccc4oc5ccccc5c4c3)cc12 ; c1ccc2c(c1)cc(-c1ccc(N(c3ccc4oc5ccccc5c4c3)c3ccc4oc5ccccc5c4c3)cc1)c1ccccc12]

Compound sub2-A-1 (10 g, 34.6 mmol), Compound sub2-18 (12.1 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14.4 g of Compound 2-18. (Yield: 69%, MS: [M+H]⁺=602)

### Synthesis Example 2-19

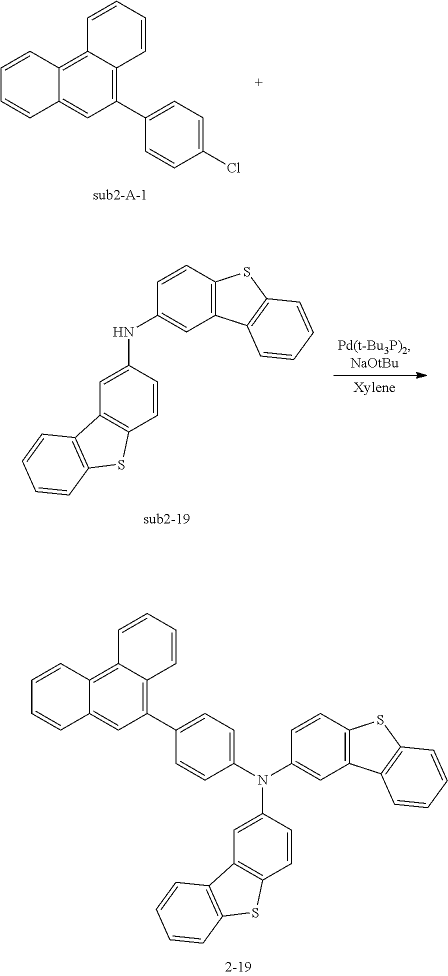 [SMILES: Clc1ccc(-c2cc3ccccc3c3ccccc23)cc1 ; c1ccc2c(c1)sc1ccc(Nc3ccc4sc5ccccc5c4c3)cc12 ; c1ccc2c(c1)cc(-c1ccc(N(c3ccc4sc5ccccc5c4c3)c3ccc4sc5ccccc5c4c3)cc1)c1ccccc12]

Compound sub2-A-1 (10 g, 34.6 mmol), Compound sub2-19 (13.2 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.4 g of Compound 2-19. (Yield: 52%, MS: [M+H]⁺=634)

### Synthesis Example 2-20

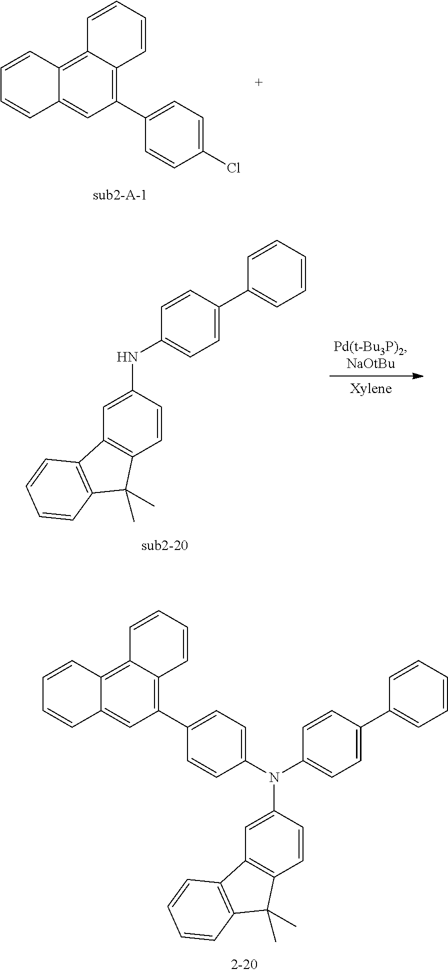 [SMILES: Clc1ccc(-c2cc3ccccc3c3ccccc23)cc1 ; CC1(C)c2ccccc2-c2cc(Nc3ccc(-c4ccccc4)cc3)ccc21 ; CC1(C)c2ccccc2-c2cc(N(c3ccc(-c4ccccc4)cc3)c3ccc(-c4cc5ccccc5c5ccccc45)cc3)ccc21]

Compound sub2-A-1 (10 g, 34.6 mmol), Compound sub2-20 (12.5 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.2 g of Compound 2-20. (Yield: 62%, MS: [M+H]⁺=614)

### Synthesis Example 2-21

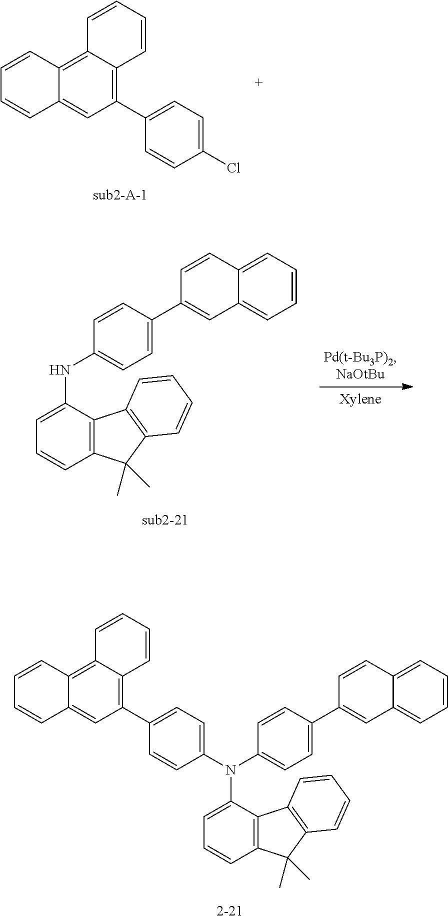 [SMILES: Clc1ccc(-c2cc3ccccc3c3ccccc23)cc1 ; CC1(C)c2ccccc2-c2c(Nc3ccc(-c4ccc5ccccc5c4)cc3)cccc21 ; CC1(C)c2ccccc2-c2c(N(c3ccc(-c4ccc5ccccc5c4)cc3)c3ccc(-c4cc5ccccc5c5ccccc45)cc3)cccc21]

Compound sub2-A-1 (10 g, 34.6 mmol), Compound sub2-21 (14.3 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14.2 g of Compound 2-21. (Yield: 62%, MS: [M+H]⁺=664)

### Synthesis Example 2-22

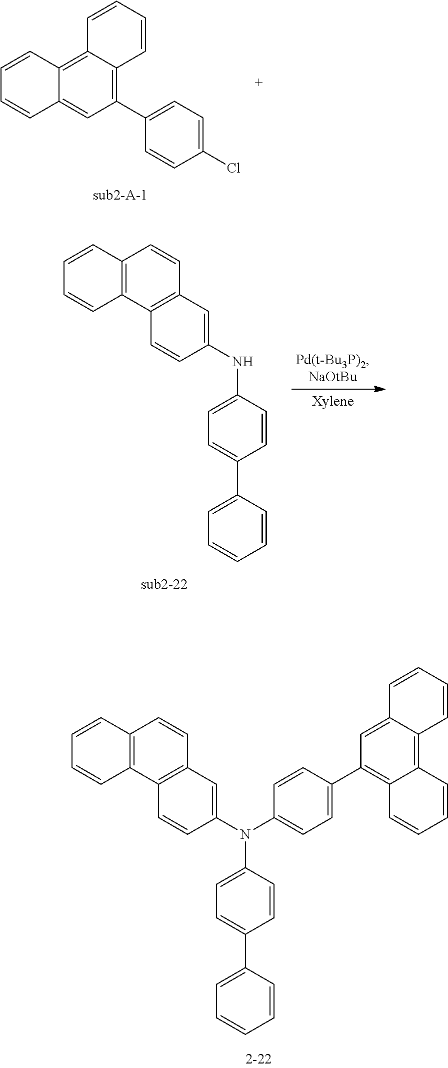 [SMILES: Clc1ccc(-c2cc3ccccc3c3ccccc23)cc1 ; c1ccc(-c2ccc(Nc3ccc4c(ccc5ccccc54)c3)cc2)cc1 ; c1ccc(-c2ccc(N(c3ccc(-c4cc5ccccc5c5ccccc45)cc3)c3ccc4c(ccc5ccccc54)c3)cc2)cc1]

Compound sub2-A-1 (10 g, 34.6 mmol), Compound sub2-22 (12 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.2 g of Compound 2-22. (Yield: 54%, MS: [M+H]⁺=598)

### Synthesis Example 2-23

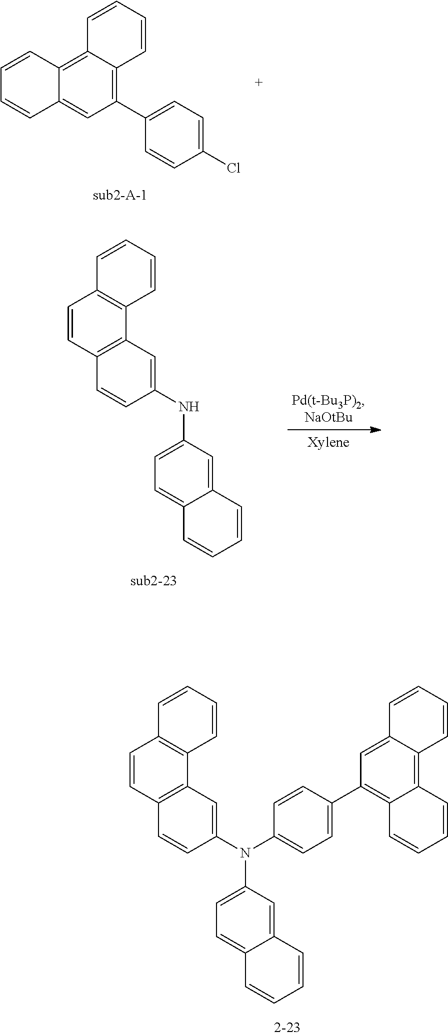 [SMILES: Clc1ccc(-c2cc3ccccc3c3ccccc23)cc1 ; c1ccc2cc(Nc3ccc4ccc5ccccc5c4c3)ccc2c1 ; c1ccc2cc(N(c3ccc(-c4cc5ccccc5c5ccccc45)cc3)c3ccc4ccc5ccccc5c4c3)ccc2c1]

Compound sub2-A-1 (10 g, 34.6 mmol), Compound sub2-23 (11.1 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.9 g of Compound 2-23. (Yield: 60%, MS: [M+H]⁺=572)

### Synthesis Example 2-24

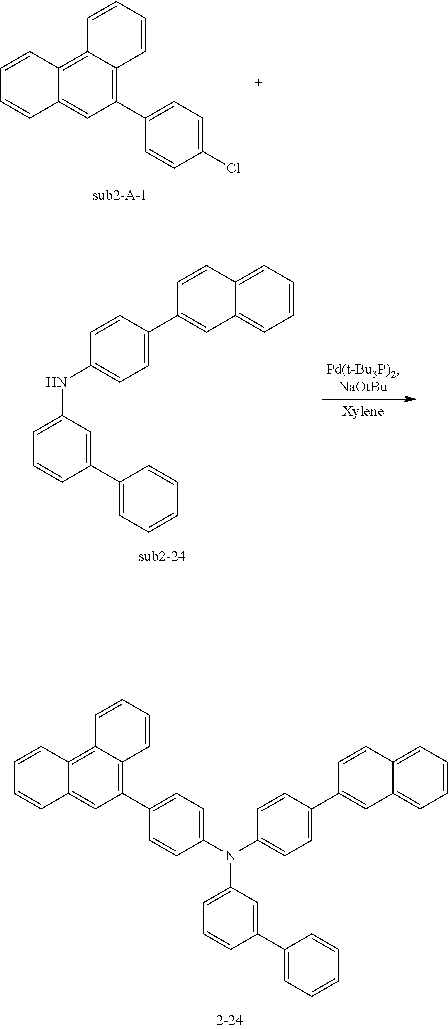 [SMILES: Clc1ccc(-c2cc3ccccc3c3ccccc23)cc1 ; c1ccc(-c2cccc(Nc3ccc(-c4ccc5ccccc5c4)cc3)c2)cc1 ; c1ccc(-c2cccc(N(c3ccc(-c4ccc5ccccc5c4)cc3)c3ccc(-c4cc5ccccc5c5ccccc45)cc3)c2)cc1]

Compound sub2-A-1 (10 g, 34.6 mmol), Compound sub2-24 (12.9 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.6 g of Compound 2-24. (Yield: 63%, MS: [M+H]⁺=624)

### Synthesis Example 2-25

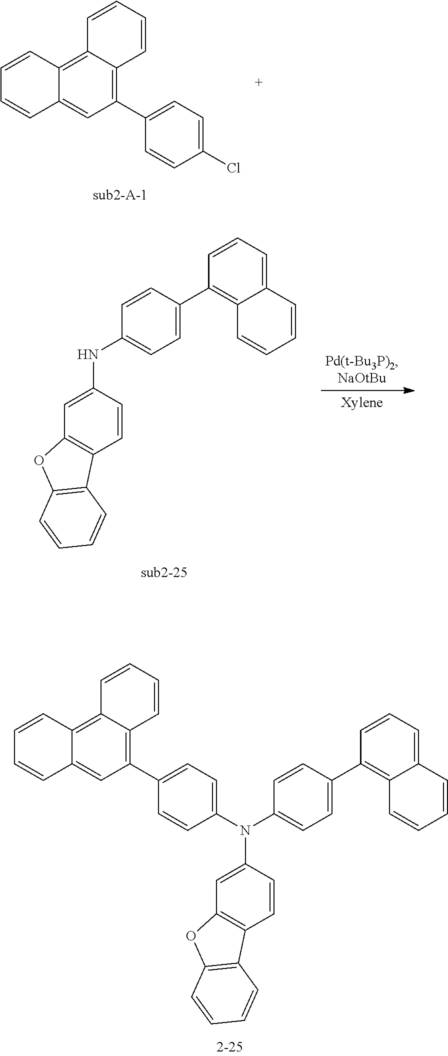 [SMILES: Clc1ccc(-c2cc3ccccc3c3ccccc23)cc1 ; c1ccc2c(-c3ccc(Nc4ccc5c(c4)oc4ccccc45)cc3)cccc2c1 ; c1ccc2c(-c3ccc(N(c4ccc(-c5cc6ccccc6c6ccccc56)cc4)c4ccc5c(c4)oc4ccccc45)cc3)cccc2c1]

Compound sub2-A-1 (10 g, 34.6 mmol), Compound sub2-25 (13.3 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(O) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14.3 g of Compound 2-25. (Yield: 65%, MS: [M+H]⁺=638)

### Synthesis Example 2-26

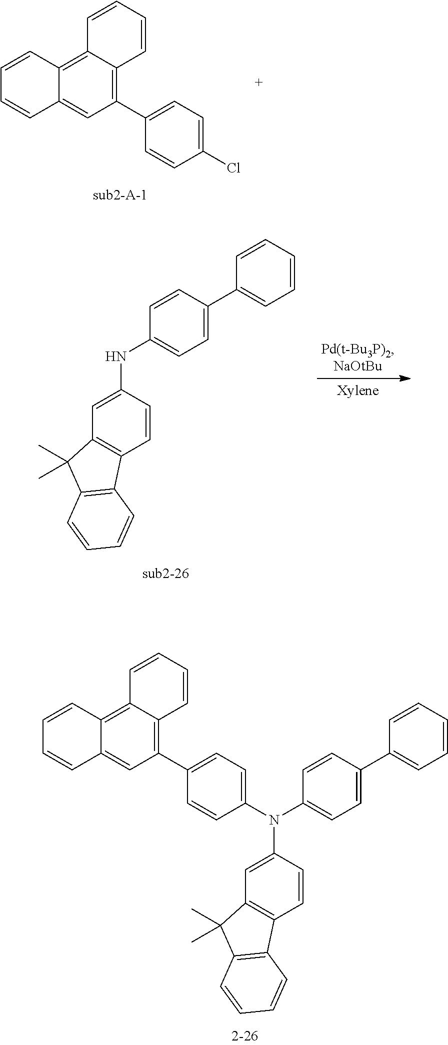 [SMILES: Clc1ccc(-c2cc3ccccc3c3ccccc23)cc1 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc(-c4cc5ccccc5c5ccccc45)cc3)cc21]

Compound sub2-A-1 (10 g, 34.6 mmol), Compound sub2-26 (12.5 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(O) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 10.8 g of Compound 2-26. (Yield: 51%, MS: [M+H]⁺=614)

### Synthesis Example 2-27

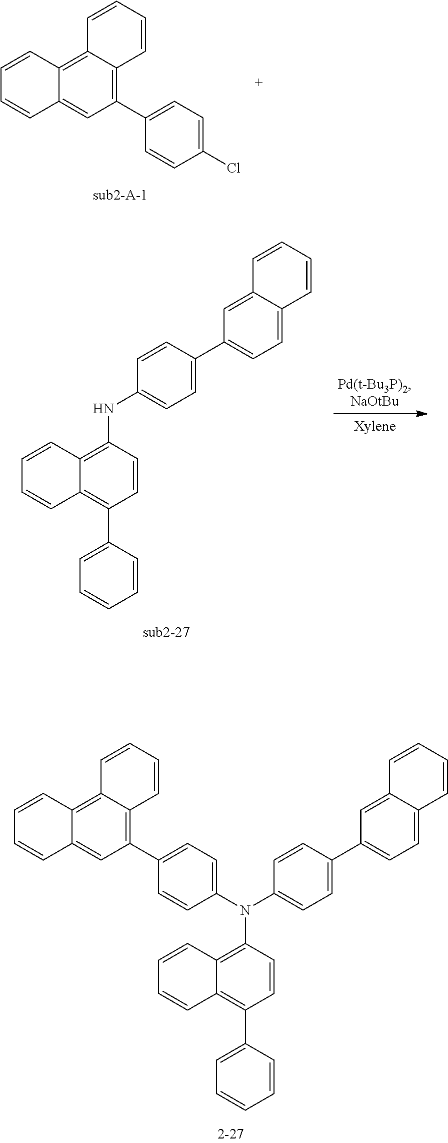 [SMILES: Clc1ccc(-c2cc3ccccc3c3ccccc23)cc1 ; c1ccc(-c2ccc(Nc3ccc(-c4ccc5ccccc5c4)cc3)c3ccccc23)cc1 ; c1ccc(-c2ccc(N(c3ccc(-c4ccc5ccccc5c4)cc3)c3ccc(-c4cc5ccccc5c5ccccc45)cc3)c3ccccc23)cc1]

Compound sub2-A-1 (10 g, 34.6 mmol), Compound sub2-27 (14.6 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 16.1 g of Compound 2-27. (Yield: 69%, MS: [M+H]⁺=674)

### Synthesis Example 2-28

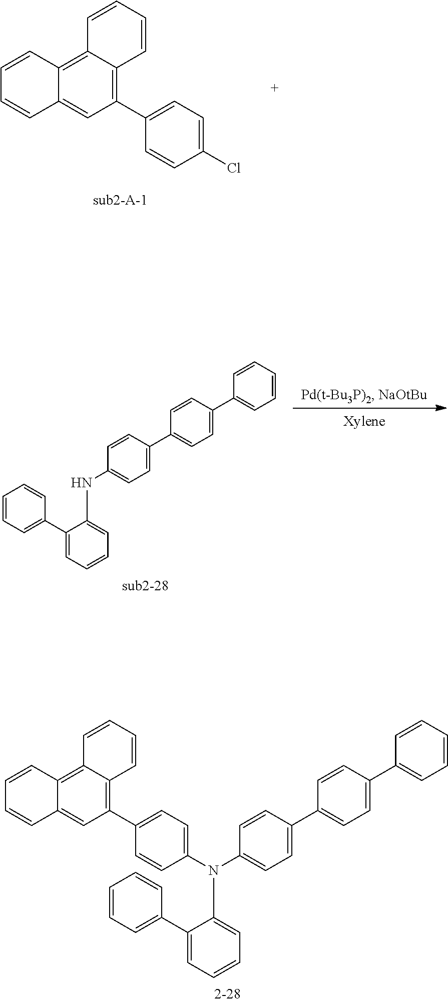

Compound sub2-A-1 (10 g, 34.6 mmol), Compound sub2-28 (13.8 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.2 g of Compound 2-28. (Yield: 50%, MS: [M+H]⁺=650)

### Synthesis Example 2-29

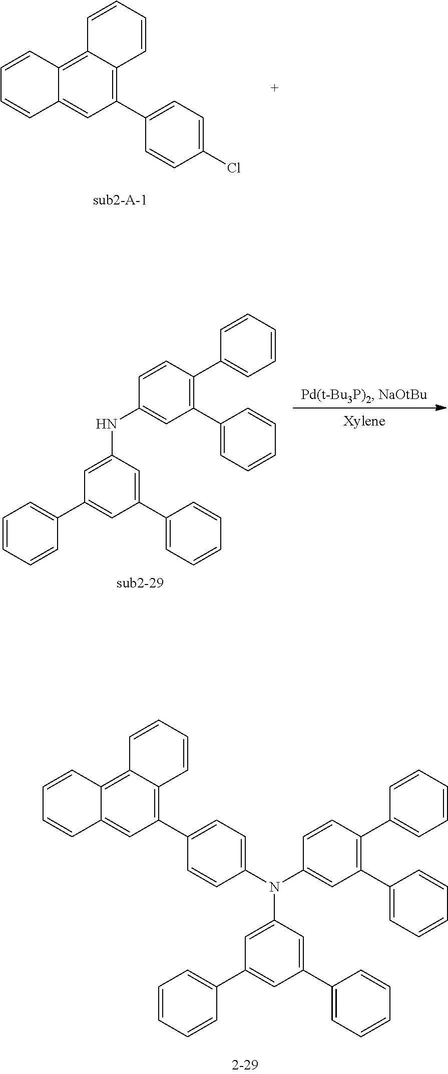

Compound sub2-A-1 (10 g, 34.6 mmol), Compound sub2-29 (16.4 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 17.1 g of Compound 2-29. (Yield: 68%, MS: [M+H]⁺=726)

### Synthesis Example 2-30

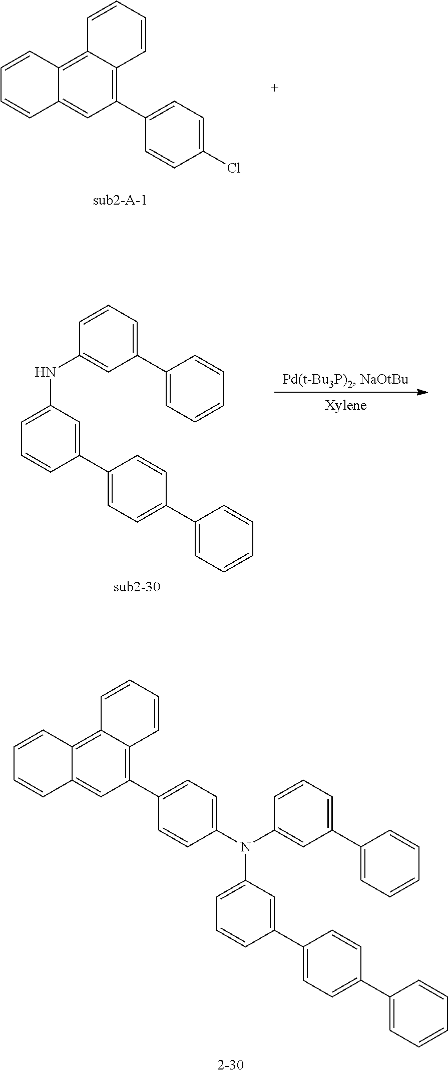

Compound sub2-A-1 (10 g, 34.6 mmol), Compound sub2-30 (13.8 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 14.4 g of Compound 2-30. (Yield: 64%, MS: [M+H]⁺=650)

### Synthesis Example 2-31

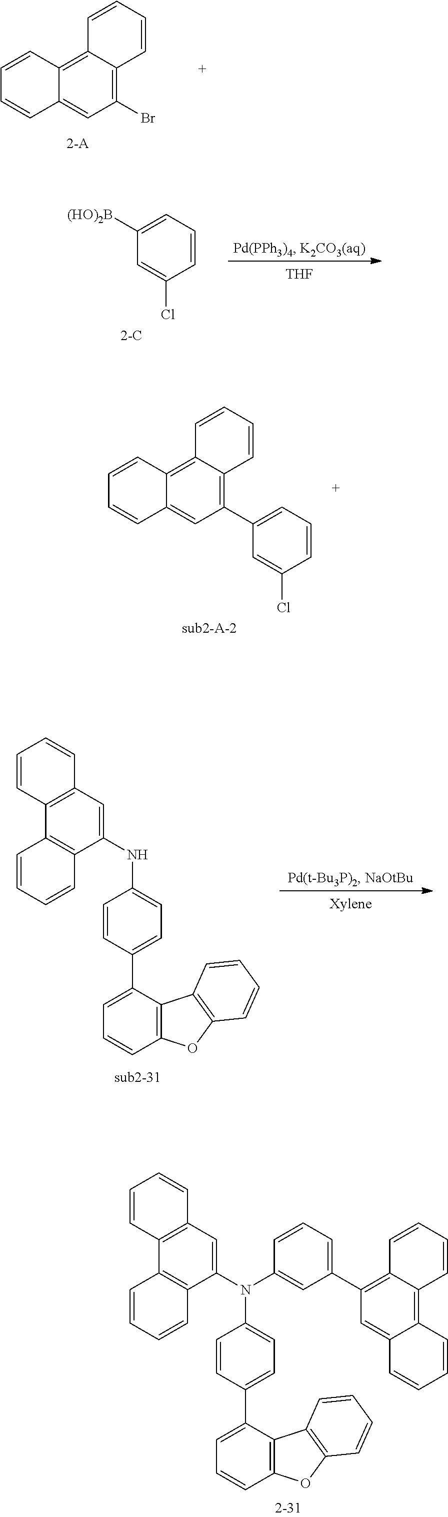

Compound 2-A (15 g, 58.3 mmol) and Compound 2-C (10 g, 64.2 mmol) were added to 300 ml of THE under a nitrogen atmosphere, and the mixture was stirred and refluxed. Then, potassium carbonate (16.1 g, 116.7 mmol) was dissolved in 48 ml of water and added thereto, and the mixture was sufficiently stirred and then tetrakis(triphenylphosphine)palladium(0) (1.3 g, 1.2 mmol) was added. After reacting for 10 hours, the reaction mixture was cooled to room temperature, then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 10.6 g of Compound sub2-A-2. (Yield: 63%, MS: [M+H]⁺=289)

Compound sub2-A-2 (10 g, 34.6 mmol), Compound sub2-31 (15.1 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 16.7 g of Compound 2-31. (Yield: 70%, MS: [M+H]⁺=688)

### Synthesis Example 2-32

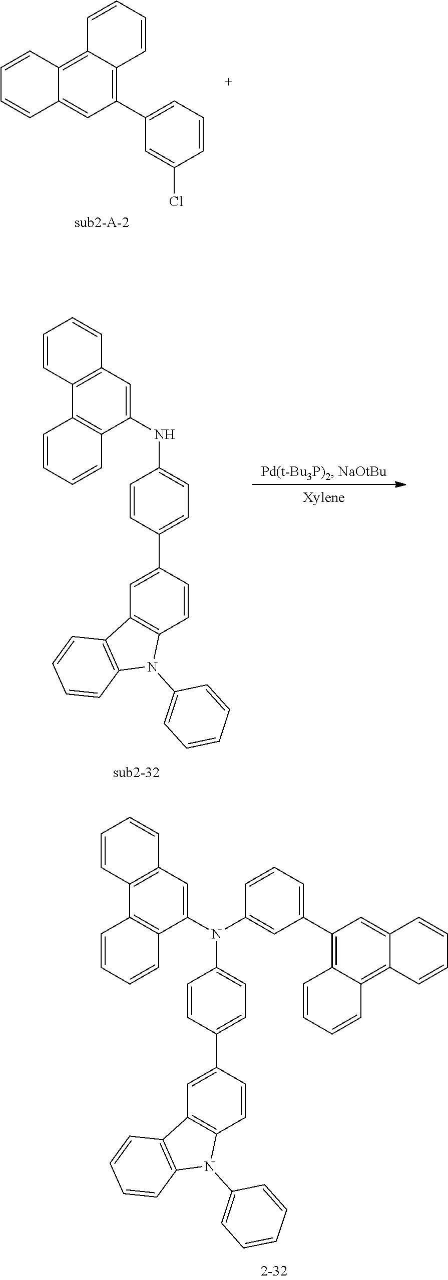

Compound sub2-A-2 (10 g, 34.6 mmol), Compound sub2-32 (17.7 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 16.6 g of Compound 2-32. (Yield: 63%, MS: [M+H]⁺=763)

### Synthesis Example 2-33

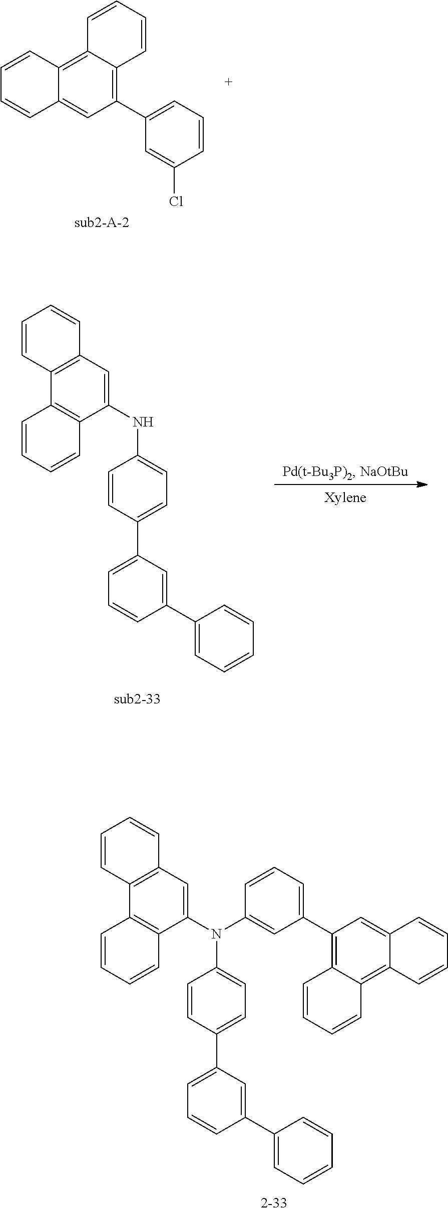

Compound sub2-A-2 (10 g, 34.6 mmol), Compound sub2-33 (14.6 g, 34.6 mmol), and sodium tert-butoxide (4.3 g, 45 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.6 g of Compound 2-33. (Yield: 54%, MS: [M+H]⁺=674)

### Synthesis Example 2-34

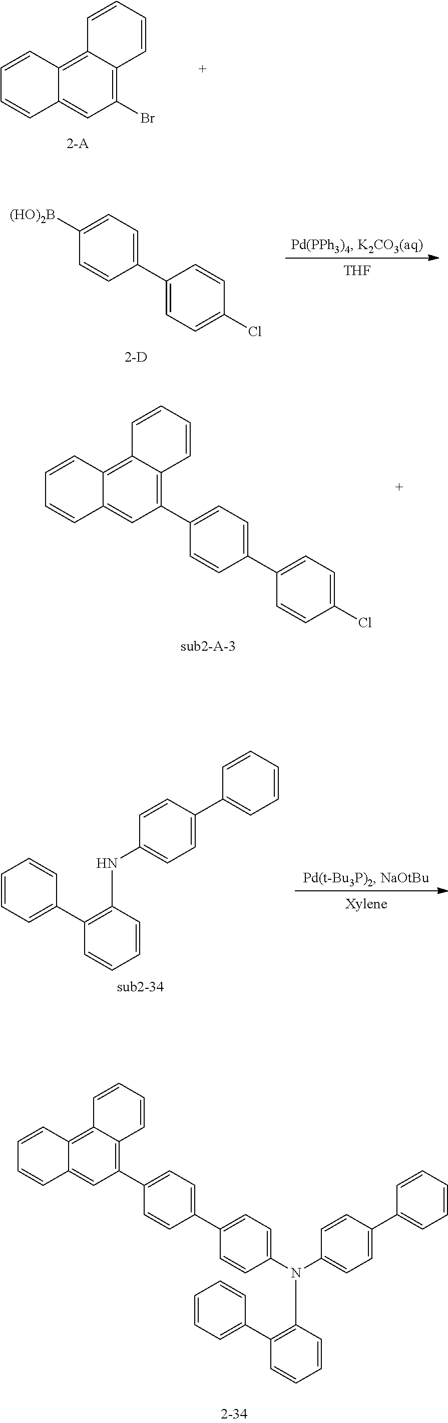

Compound 2-A (15 g, 58.3 mmol) and Compound 2-D (14.9 g, 64.2 mmol) were added to 300 ml of THE under a nitrogen atmosphere, and the mixture was stirred and refluxed. Then, potassium carbonate (16.1 g, 116.7 mmol) was dissolved in 48 ml of water and added thereto, and the mixture was sufficiently stirred and then tetrakis(triphenylphosphine)palladium(0) (1.3 g, 1.2 mmol) was added. After reacting for 10 hours, the reaction mixture was cooled to room temperature, then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.8 g of Compound sub2-A-3. (Yield: 79%, MS: [M+H]⁺=365)

Compound sub2-A-3 (10 g, 27.4 mmol), Compound sub2-34 (8.8 g, 27.4 mmol), and sodium tert-butoxide (3.4 g, 35.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.2 g of Compound 2-34. (Yield: 63%, MS: [M+H]⁺=650)

### Synthesis Example 2-35

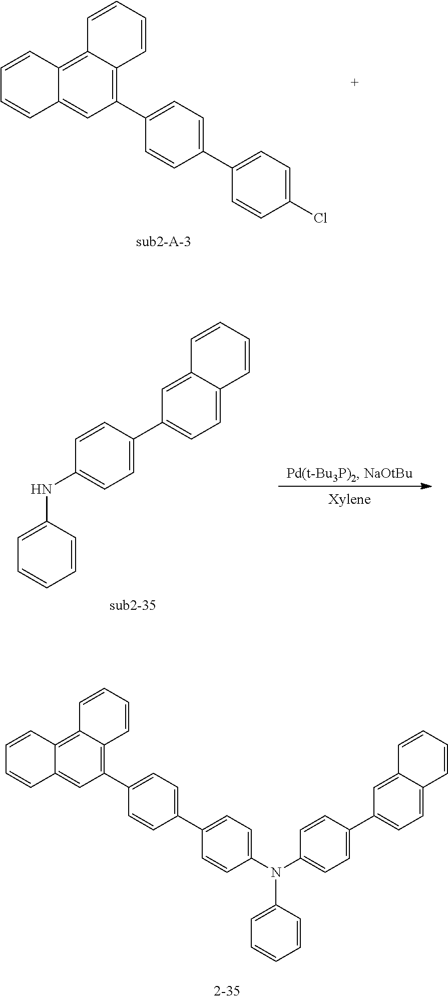

Compound sub2-A-3 (10 g, 27.4 mmol), Compound sub2-35 (8.1 g, 27.4 mmol), and sodium tert-butoxide (3.4 g, 35.7 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 8.7 g of Compound 2-35. (Yield: 51%, MS: [M+H]⁺=624)

### Synthesis Example 2-36

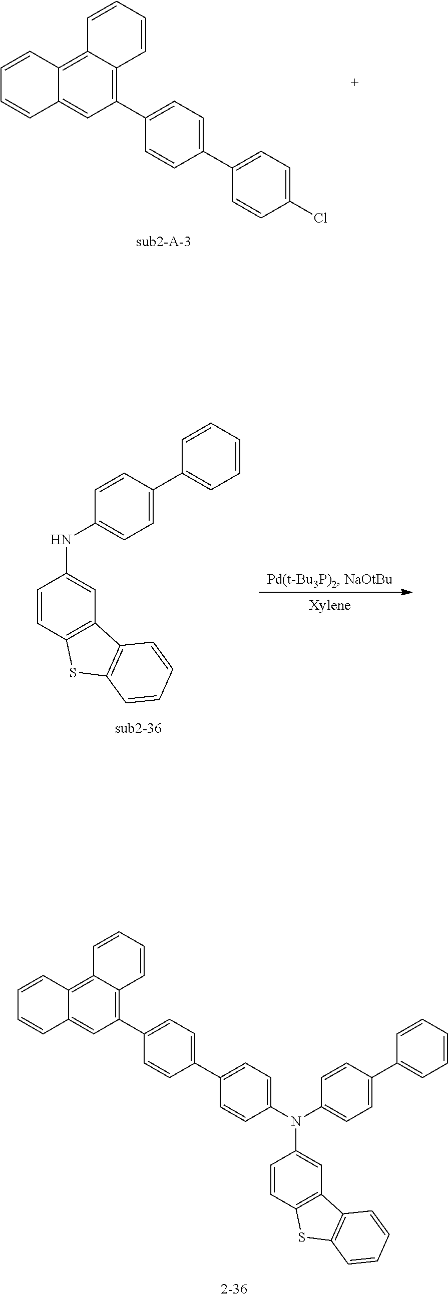

Compound sub2-A-3 (10 g, 27.4 mmol), Compound sub2-36 (9.6 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.1 g of Compound 2-36. (Yield: 65%, MS: [M+H]⁺=680)

### Synthesis Example 2-37

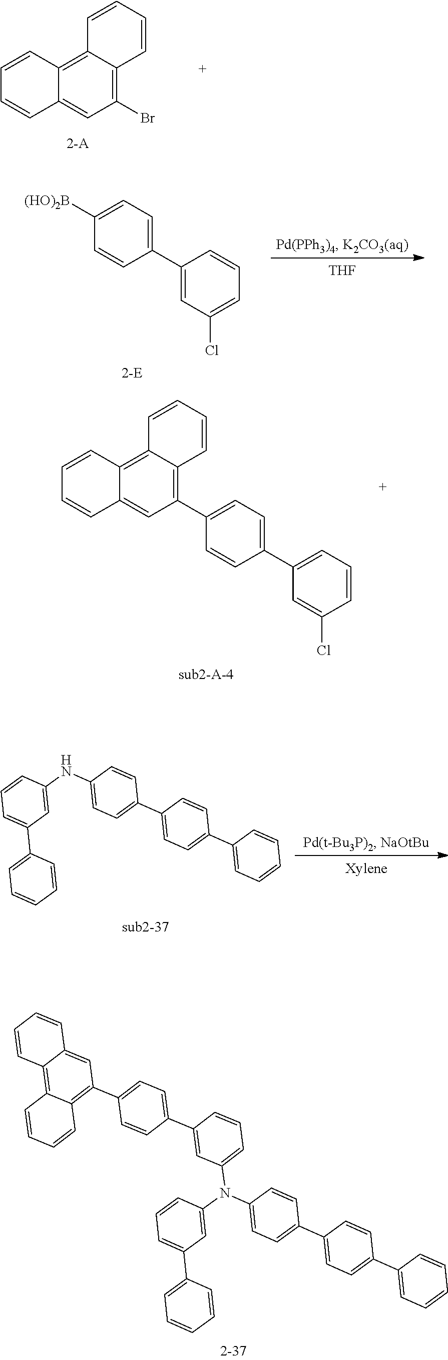

Compound 2-A (15 g, 58.3 mmol) and Compound 2-E (14.9 g, 64.2 mmol) were added to 300 ml of THE under a nitrogen atmosphere, and the mixture was stirred and refluxed. Then, potassium carbonate (16.1 g, 116.7 mmol) was dissolved in 48 ml of water and added thereto, and the mixture was sufficiently stirred and then tetrakis(triphenylphosphine)palladium(0) (1.3 g, 1.2 mmol) was added. After reacting for 11 hours, the reaction mixture was cooled to room temperature, then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.2 g of Compound sub2-A-4. (Yield: 67%, MS: [M+H]⁺=365)

Compound sub2-A-4 (10 g, 27.4 mmol), Compound sub2-37 (10.9 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.9 g of Compound 2-37. (Yield: 70%, MS: [M+H]⁺=726)

### Synthesis Example 2-38

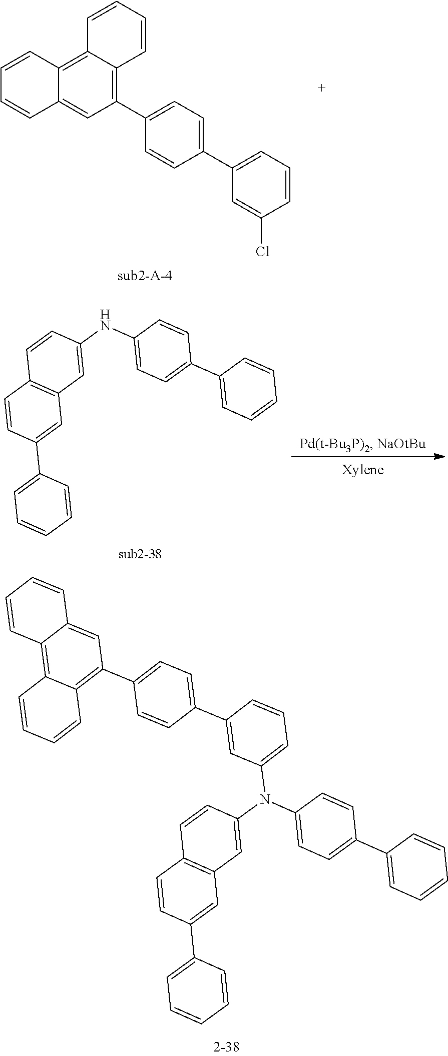

Compound sub2-A-4 (10 g, 27.4 mmol), Compound sub2-38 (10.2 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 10.7 g of Compound 2-38. (Yield: 56%, MS: [M+H]⁺=700)

### Synthesis Example 2-39

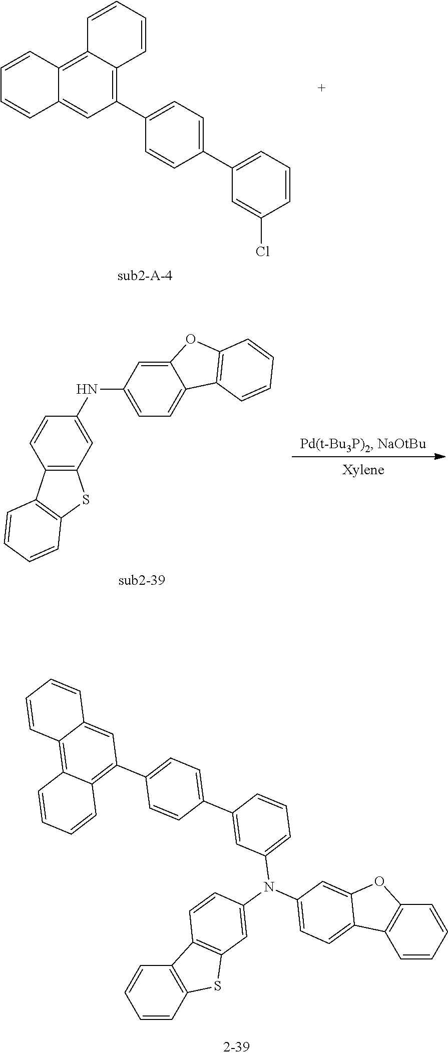

Compound sub2-A-4 (10 g, 27.4 mmol), Compound sub2-39 (10 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.8 g of Compound 2-39. (Yield: 62%, MS: [M+H]⁺=694)

### Synthesis Example 2-40

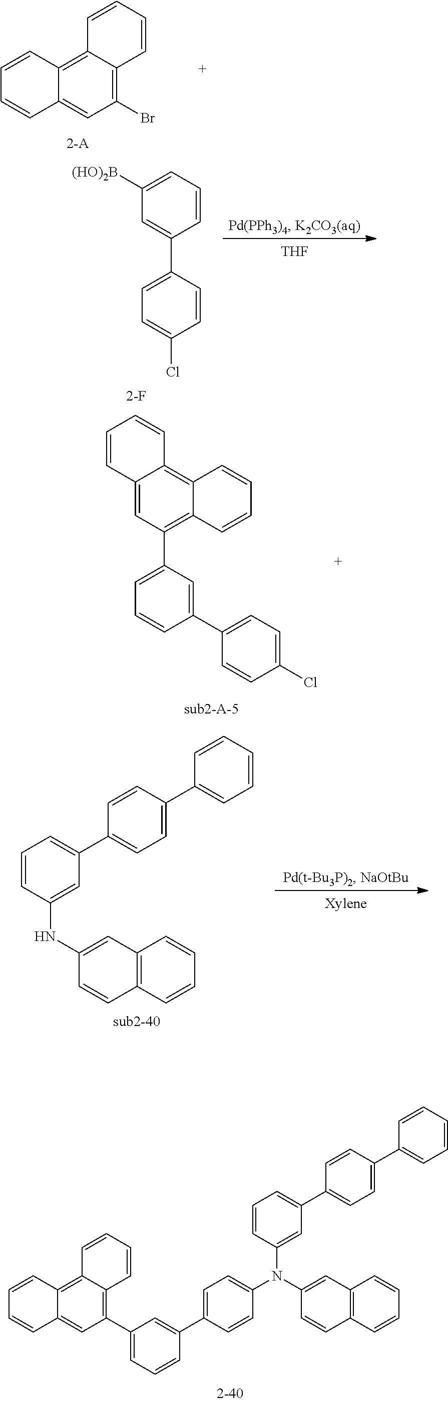

Compound 2-A (15 g, 58.3 mmol) and Compound 2-F (14.9 g, 64.2 mmol) were added to 300 ml of THE under a nitrogen atmosphere, and the mixture was stirred and refluxed. Then, potassium carbonate (16.1 g, 116.7 mmol) was dissolved in 48 ml of water and added thereto, and the mixture was sufficiently stirred and then tetrakis(triphenylphosphine)palladium(0) (1.3 g, 1.2 mmol) was added. After reacting for 8 hours, the reaction mixture was cooled to room temperature, then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.4 g of Compound sub2-A-5. (Yield: 68%, MS: [M+H]⁺=365)

Compound sub2-A-5 (10 g, 27.4 mmol), Compound sub2-40 (10.2 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 10.7 g of Compound 2-40. (Yield: 56%, MS: [M+H]⁺=700)

### Synthesis Example 2-41

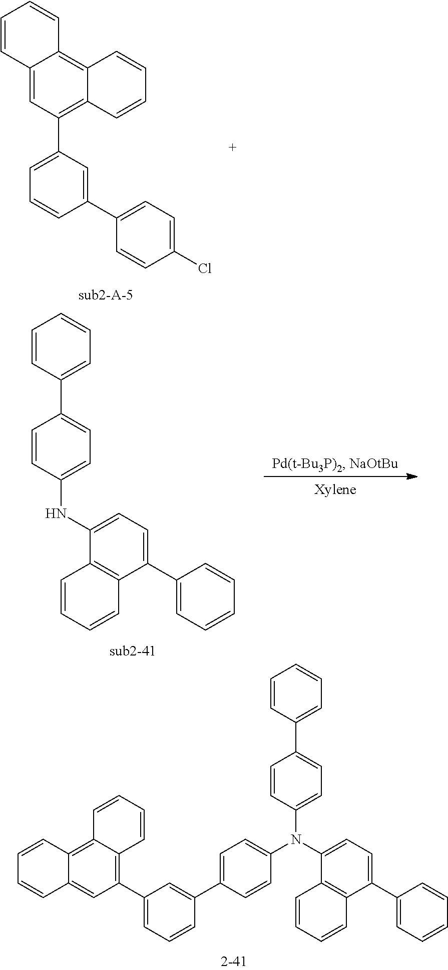

Compound sub2-A-5 (10 g, 27.4 mmol), Compound sub2-41 (10.2 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 9.8 g of Compound 2-41. (Yield: 51%, MS: [M+H]⁺=700)

### Synthesis Example 2-42

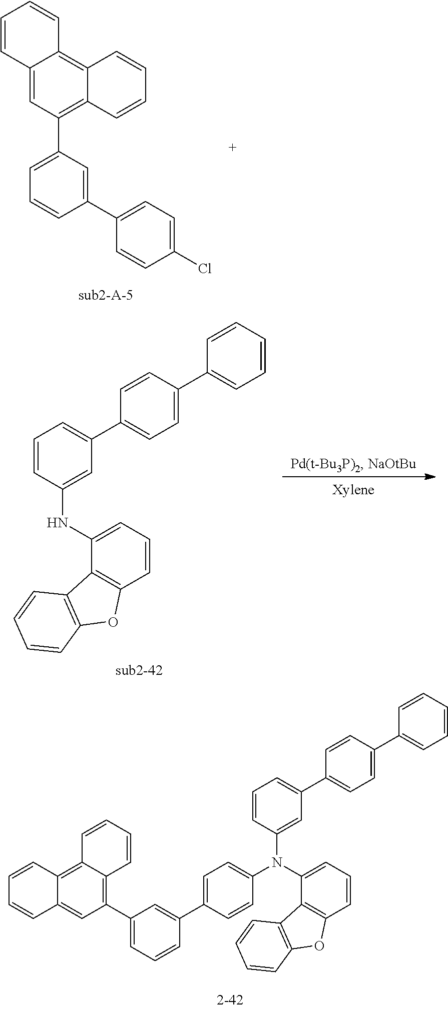

Compound sub2-A-5 (10 g, 27.4 mmol), Compound sub2-42 (11.3 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.5 g of Compound 2-42. (Yield: 57%, MS: [M+H]⁺=740)

### Synthesis Example 2-43

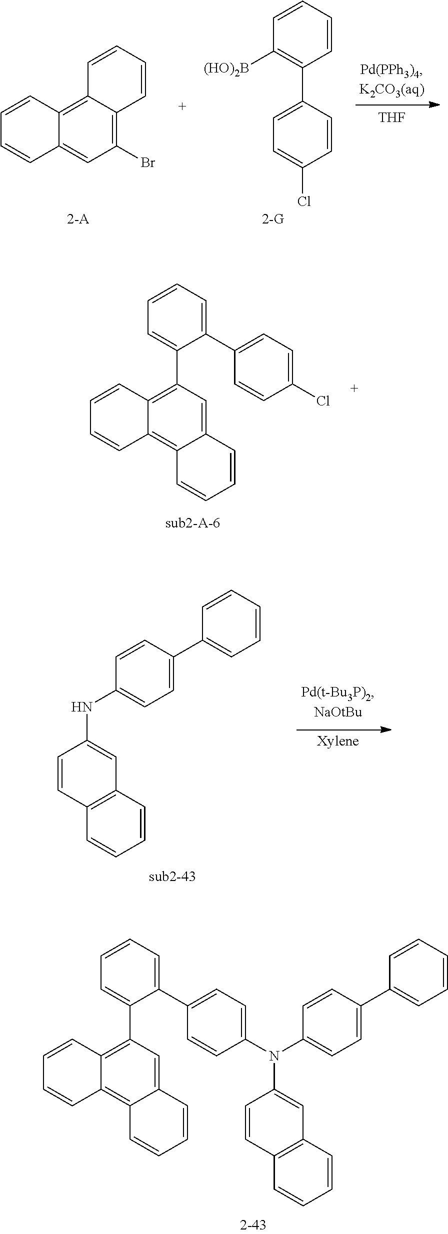

Compound 2-A (15 g, 58.3 mmol) and Compound 2-G (14.9 g, 64.2 mmol) were added to 300 ml of THE under a nitrogen atmosphere, and the mixture was stirred and refluxed. Then, potassium carbonate (16.1 g, 116.7 mmol) was dissolved in 48 ml of water and added thereto, and the mixture was sufficiently stirred and then tetrakis(triphenylphosphine)palladium(0) (1.3 g, 1.2 mmol) was added. After reacting for 9 hours, the reaction mixture was cooled to room temperature, then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.7 g of Compound sub2-A-6. (Yield: 69%, MS: [M+H]⁺=365)

Compound sub2-A-6 (10 g, 27.4 mmol), Compound sub2-43 (8.1 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 9.7 g of Compound 2-43. (Yield: 57%, MS: [M+H]⁺=624)

### Synthesis Example 2-44

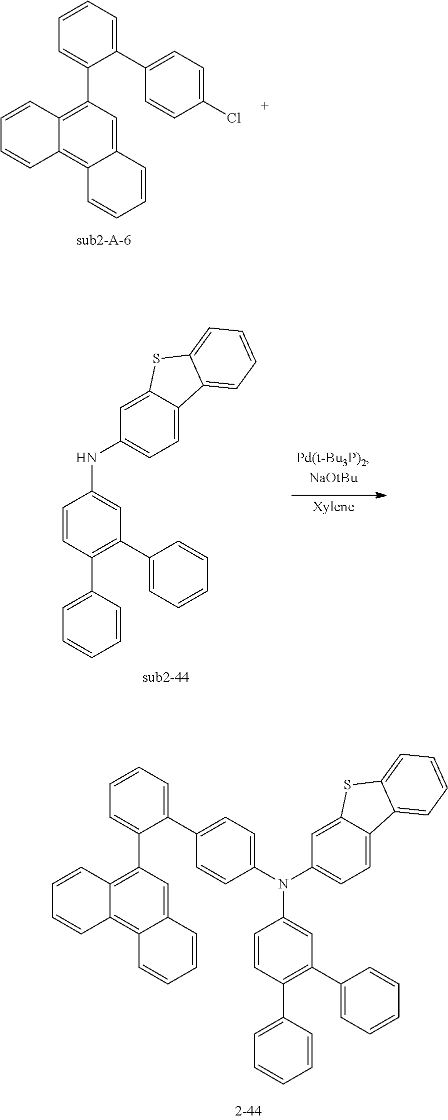 [SMILES: Clc1ccc(-c2ccccc2-c2cc3ccccc3c3ccccc23)cc1 ; c1ccc(-c2ccc(Nc3ccc4c(c3)sc3ccccc34)cc2-c2ccccc2)cc1 ; "Xylene" ; c1ccc(-c2ccc(N(c3ccc(-c4ccccc4-c4cc5ccccc5c5ccccc45)cc3)c3ccc4c(c3)sc3ccccc34)cc2-c2ccccc2)cc1]

Compound sub2-A-6 (10 g, 27.4 mmol), Compound sub2-44 (11.7 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12 g of Compound 2-44. (Yield: 58%, MS: [M+H]⁺=756)

### Synthesis Example 2-45

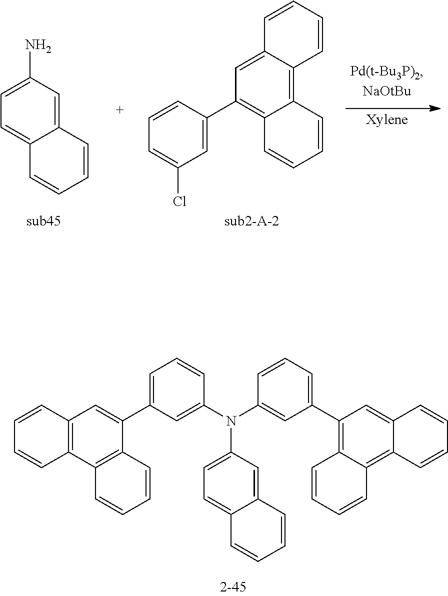 [SMILES: Nc1ccc2ccccc2c1 ; Clc1cccc(-c2cc3ccccc3c3ccccc23)c1 ; "Xylene" ; c1cc(-c2cc3ccccc3c3ccccc23)cc(N(c2cccc(-c3cc4ccccc4c4ccccc34)c2)c2ccc3ccccc3c2)c1]

Compound sub45 (10 g, 70.3 mmol), Compound sub2-A-2 (42.6 g, 147.7 mmol), and sodium tert-butoxide (31.0 g, 322.6 mmol) were added to 200 ml of xylene under a nitrogen atmosphere, and the mixture was stirred and refluxed. Then, bis(tri-tert-butylphosphine)palladium(0) (1.5 g, 2.9 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 31 g of Compound 2-45. (Yield: 68%, MS: [M+H]⁺=648)

### Synthesis Example 2-46

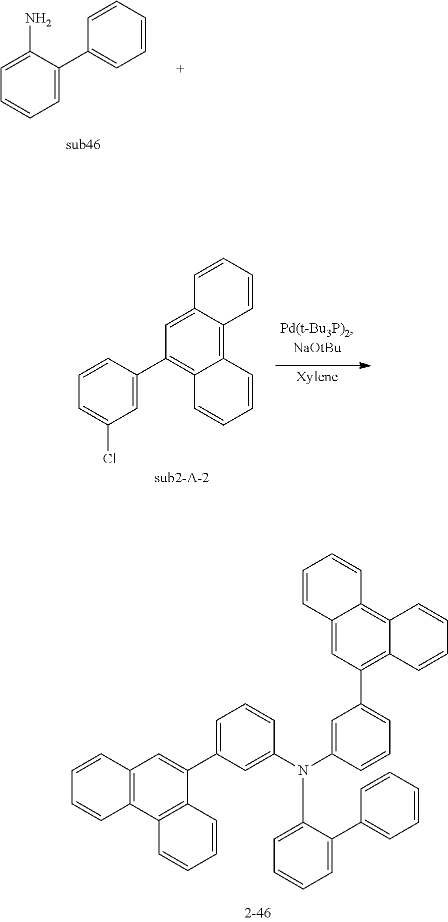 [SMILES: Nc1ccccc1-c1ccccc1 ; Clc1cccc(-c2cc3ccccc3c3ccccc23)c1 ; "Xylene" ; c1ccc(-c2ccccc2N(c2cccc(-c3cc4ccccc4c4ccccc34)c2)c2cccc(-c3cc4ccccc4c4ccccc34)c2)cc1]

Compound sub46 (10 g, 59.1 mmol), Compound sub2-A-2 (35.8 g, 124.1 mmol), and sodium tert-butoxide (14.2 g, 147.7 mmol) were added to 200 ml of xylene under a nitrogen atmosphere, and the mixture was stirred and refluxed. Then, bis(tri-tert-butylphosphine)palladium(0) (0.6 g, 1.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 26.7 g of Compound 2-46. (Yield: 67%, MS: [M+H]⁺=674)

### Synthesis Example 2-47

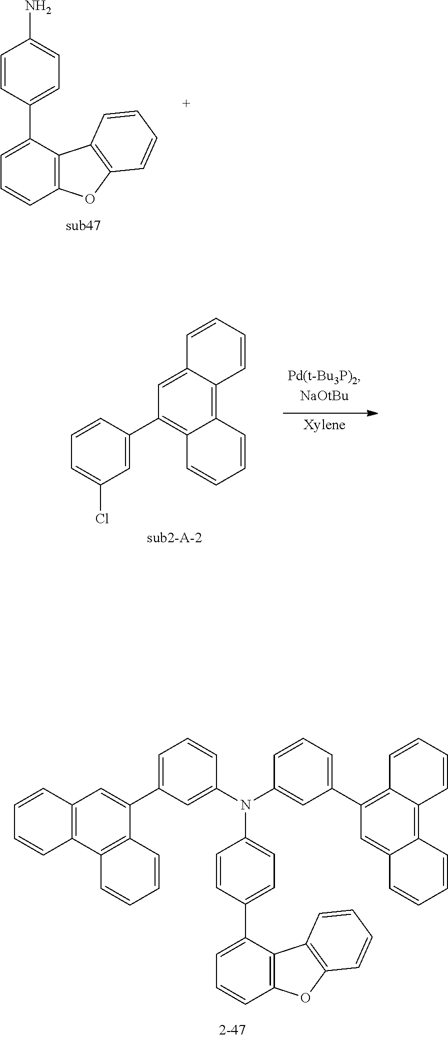 [SMILES: Nc1ccc(-c2cccc3oc4ccccc4c23)cc1 ; Clc1cccc(-c2cc3ccccc3c3ccccc23)c1 ; "Xylene" ; c1cc(-c2cc3ccccc3c3ccccc23)cc(N(c2ccc(-c3cccc4oc5ccccc5c34)cc2)c2cccc(-c3cc4ccccc4c4ccccc34)c2)c1]

Compound sub47 (10 g, 38.6 mmol), Compound sub2-A-2 (23.4 g, 81 mmol), and sodium tert-butoxide (9.3 g, 96.4 mmol) were added to 200 ml of xylene under a nitrogen atmosphere, and the mixture was stirred and refluxed. Then, bis(tri-tert-butylphosphine)palladium(0) (0.4 g, 0.8 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 15 g of Compound 2-47. (Yield: 51%, MS: [M+H]⁺=764)

### Synthesis Example 2-48

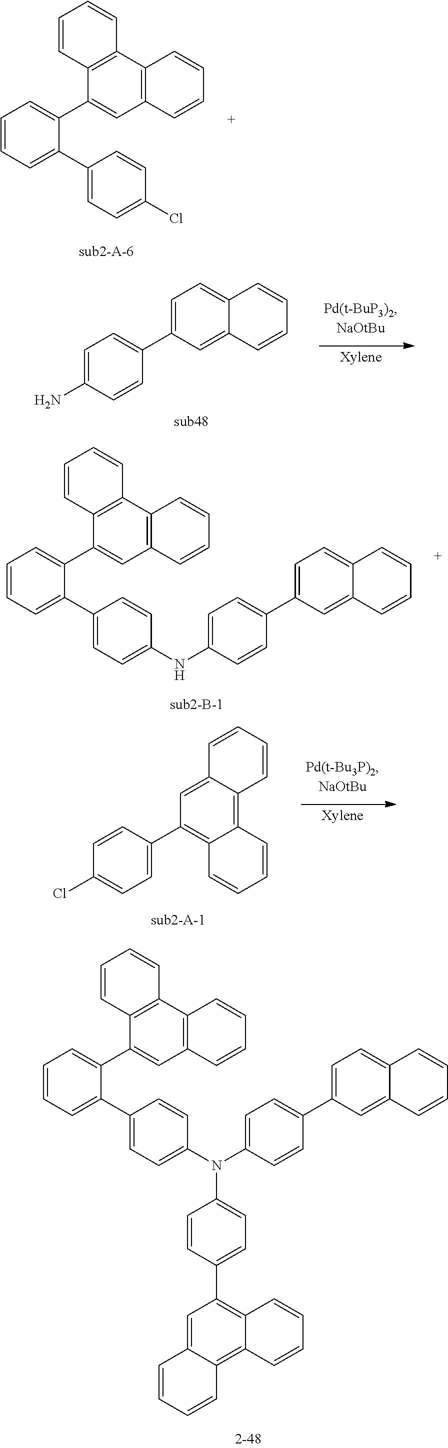

Compound sub2-A-6 (10 g, 27.4 mmol), Compound sub48 (6 g, 27.4 mmol), and sodium tert-butoxide (2.9 g, 30.1 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 9 g of Compound sub2-B-1. (Yield: 60%, MS: [M+H]⁺=548)

Compound sub2-B-1 (10 g, 18.3 mmol), Compound sub2-A-1 (5.3 g, 18.3 mmol), and sodium tert-butoxide (2.3 g, 23.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 7.7 g of Compound 2-46. (Yield: 53%, MS: [M+H]⁺=800)

### Synthesis Example 2-49

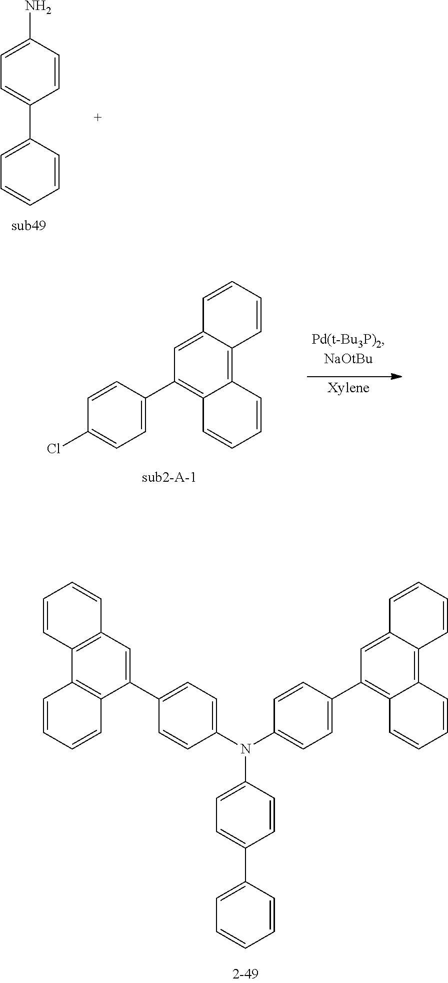 [SMILES: Nc1ccc(-c2ccccc2)cc1 ; Clc1ccc(-c2cc3ccccc3c3ccccc23)cc1 ; "Xylene" ; c1ccc(-c2ccc(N(c3ccc(-c4cc5ccccc5c5ccccc45)cc3)c3ccc(-c4cc5ccccc5c5ccccc45)cc3)cc2)cc1]

Compound sub49 (10 g, 59.1 mmol), Compound sub2-A-1 (35.8 g, 124.1 mmol), and sodium tert-butoxide (14.2 g, 147.7 mmol) were added to 200 ml of xylene under a nitrogen atmosphere, and the mixture was stirred and refluxed. Then, bis(tri-tert-butylphosphine)palladium(0) (0.6 g, 1.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 22.7 g of Compound 2-49. (Yield: 57%, MS: [M+H]⁺=674)

### Synthesis Example 2-50

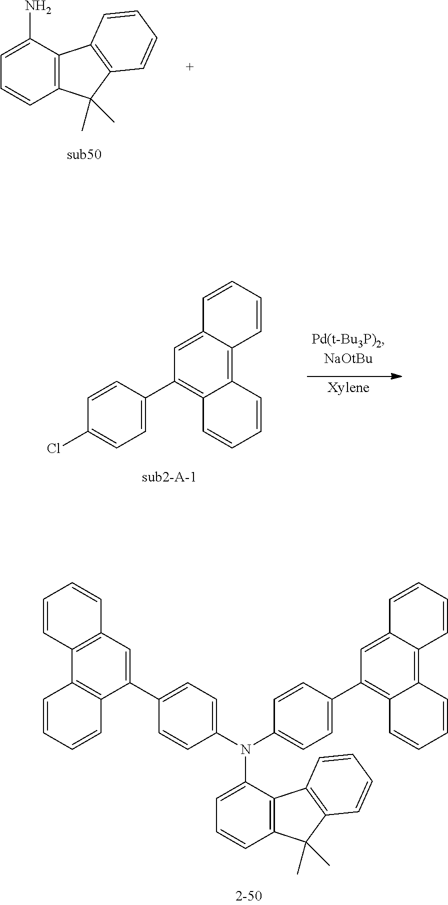 [SMILES: CC1(C)c2ccccc2-c2c(N)cccc21 ; Clc1ccc(-c2cc3ccccc3c3ccccc23)cc1 ; "Xylene" ; CC1(C)c2ccccc2-c2c(N(c3ccc(-c4cc5ccccc5c5ccccc45)cc3)c3ccc(-c4cc5ccccc5c5ccccc45)cc3)cccc21]

Compound sub50 (10 g, 47.8 mmol), Compound sub2-A-1 (29 g, 100.3 mmol), and sodium tert-butoxide (11.5 g, 119.5 mmol) were added to 200 ml of xylene under a nitrogen atmosphere, and the mixture was stirred and refluxed. Then, bis(tri-tert-butylphosphine)palladium(0) (0.5 g, 1 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 23.9 g of Compound 2-50. (Yield: 70%, MS: [M+H]⁺=714) Synthesis Example 2-51

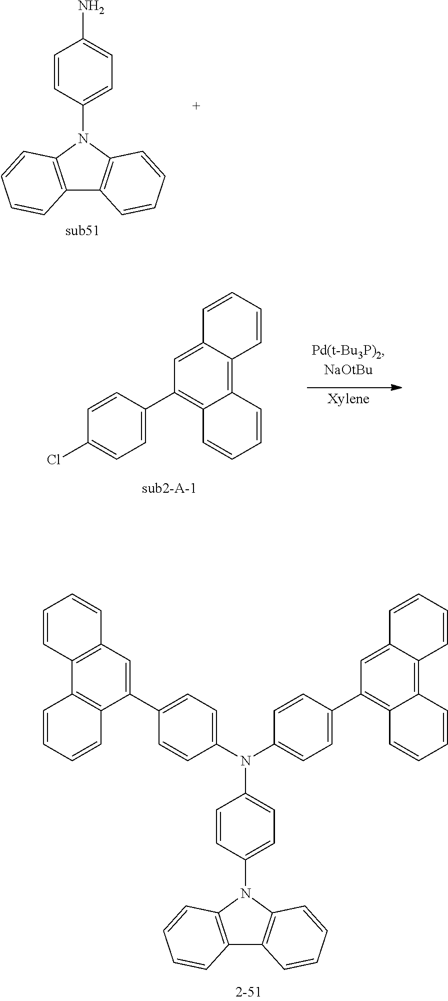 [SMILES: Nc1ccc(-n2c3ccccc3c3ccccc32)cc1 ; Clc1ccc(-c2cc3ccccc3c3ccccc23)cc1 ; "Xylene" ; c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12]

Compound sub51 (10 g, 38.7 mmol), Compound sub2-A-1 (23.5 g, 81.3 mmol), and sodium tert-butoxide (9.3 g, 96.8 mmol) were added to 200 ml of xylene under a nitrogen atmosphere, and the mixture was stirred and refluxed. Then, bis(tri-tert-butylphosphine)palladium(0) (0.4 g, 0.8 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 16.8 g of Compound 2-51. (Yield: 57%, MS: [M+H]⁺=763)

### Synthesis Example 2-52

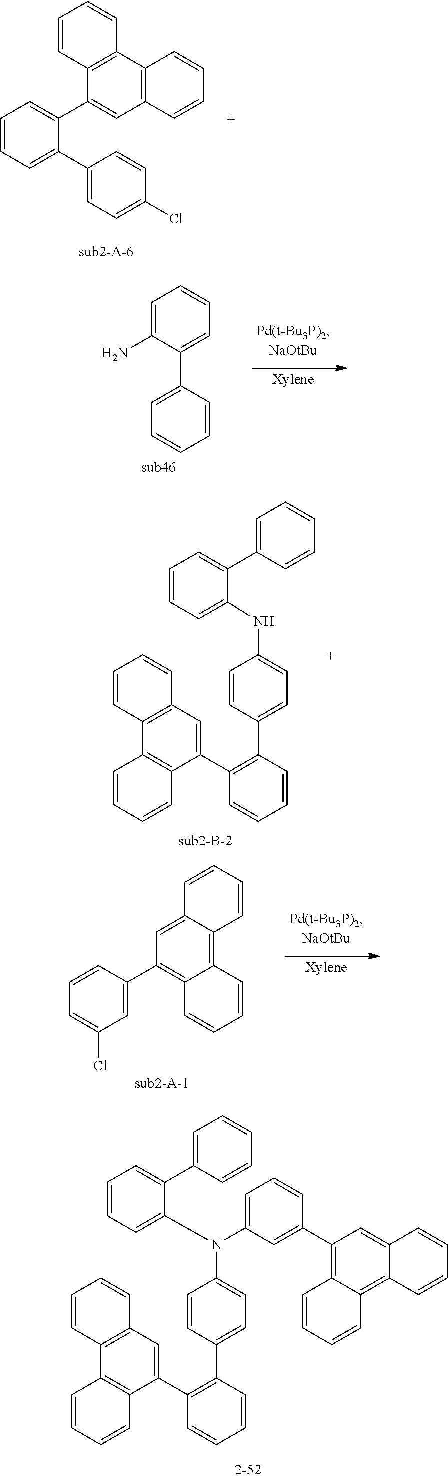

Compound sub2-A-6 (10 g, 27.4 mmol), Compound sub46 (4.6 g, 27.4 mmol), and sodium tert-butoxide (2.9 g, 30.1 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 9.4 g of Compound sub2-B-2. (Yield: 69%, MS: [M+H]⁺=498)

Compound sub2-B-2 (10 g, 20.1 mmol), Compound sub2-A-1 (5.8 g, 20.1 mmol), and sodium tert-butoxide (2.5 g, 26.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 8.3 g of Compound 2-52. (Yield: 55%, MS: [M+H]⁺=750)

### Synthesis Example 2-53

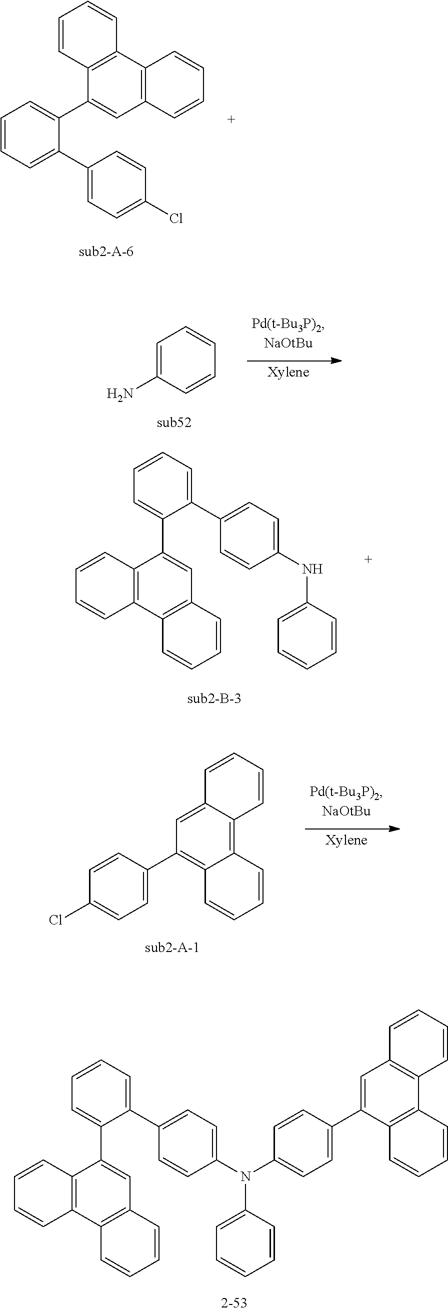

Compound sub2-A-6 (10 g, 27.4 mmol), Compound sub52 (2.6 g, 27.4 mmol), and sodium tert-butoxide (2.9 g, 30.1 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 5.9 g of Compound sub2-B-3. (Yield: 51%, MS: [M+H]⁺=422)

Compound sub2-B-3 (10 g, 23.7 mmol), Compound sub2-A-1 (6.9 g, 23.7 mmol), and sodium tert-butoxide (3 g, 30.8 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 9.3 g of Compound 2-53. (Yield: 58%, MS: [M+H]⁺=674)

### Synthesis Example 2-54

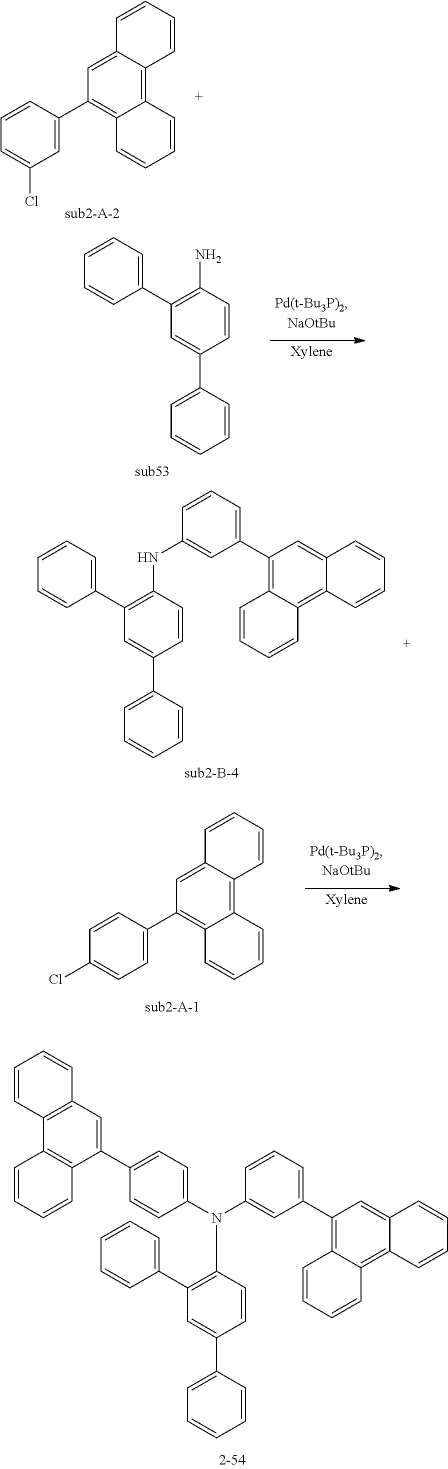

Compound sub2-A-2 (10 g, 34.6 mmol), Compound sub53 (8.5 g, 34.6 mmol), and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.5 g of Compound sub2-B-4. (Yield: 67%, MS: [M+H]⁺=498)

Compound sub2-B-4 (10 g, 20.1 mmol), Compound sub2-A-1 (5.8 g, 20.1 mmol), and sodium tert-butoxide (2.5 g, 26.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 7.5 g of Compound 2-54. (Yield: 50%, MS: [M+H]⁺=750)

### Synthesis Example 2-55

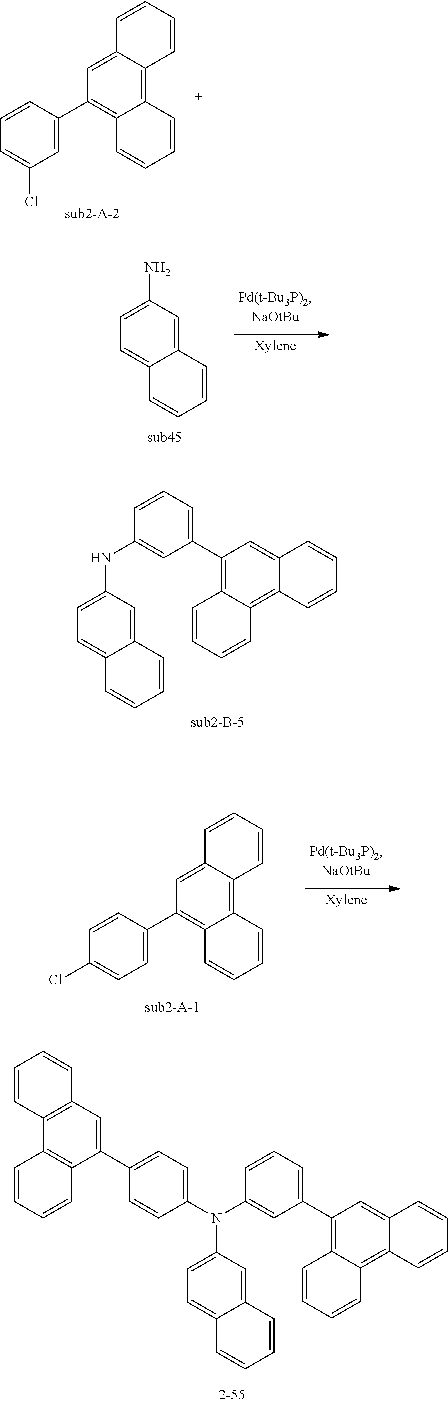

Compound sub2-A-2 (10 g, 34.6 mmol), Compound sub45 (5 g, 34.6 mmol), and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 9.3 g of Compound sub2-B-5. (Yield: 68%, MS: [M+H]⁺=396)

Compound sub2-B-5 (10 g, 25.3 mmol), Compound sub2-A-1 (7.3 g, 25.3 mmol), and sodium tert-butoxide (3.2 g, 32.9 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 10 g of Compound 2-55. (Yield: 61%, MS: [M+H]⁺=648)

### Synthesis Example 2-56

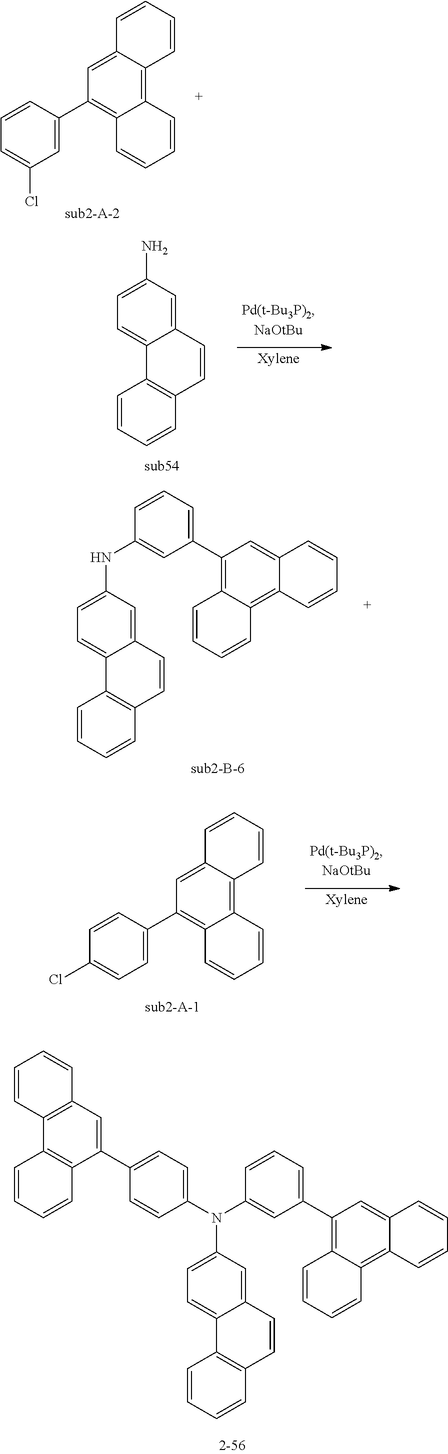

Compound sub2-A-2 (10 g, 34.6 mmol), Compound sub54 (6.7 g, 34.6 mmol), and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 8.6 g of Compound sub2-B-6. (Yield: 56%, MS: [M+H]⁺=446)

Compound sub2-B-6 (10 g, 22.4 mmol), Compound sub2-A-1 (6.5 g, 22.4 mmol), and sodium tert-butoxide (2.8 g, 29.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 8.8 g of Compound 2-56. (Yield: 56%, MS: [M+H]⁺=698)

### Synthesis Example 2-57

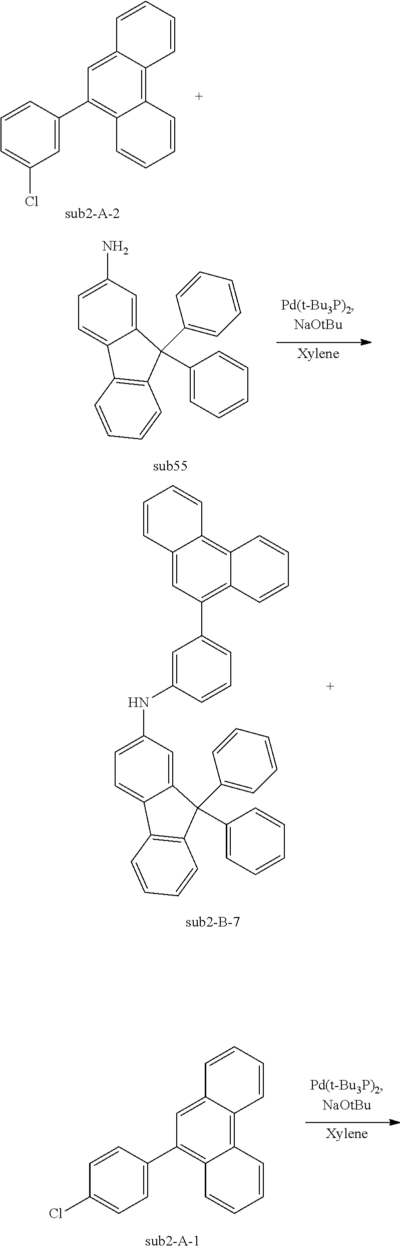 [SMILES: Clc1cccc(-c2cc3ccccc3c3ccccc23)c1 ; Nc1ccc2c(c1)C(c1ccccc1)(c1ccccc1)c1ccccc1-2 ; "Xylene" ; c1ccc(C2(c3ccccc3)c3ccccc3-c3ccc(Nc4cccc(-c5cc6ccccc6c6ccccc56)c4)cc32)cc1 ; Clc1ccc(-c2cc3ccccc3c3ccccc23)cc1 ; "Xylene"]

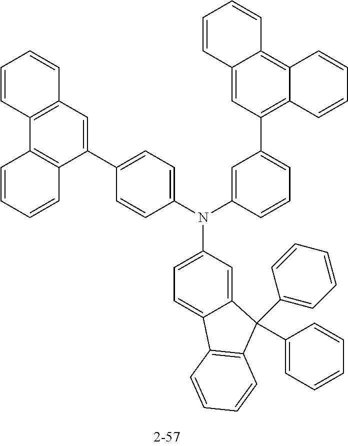 [SMILES: c1ccc(C2(c3ccccc3)c3ccccc3-c3ccc(N(c4ccc(-c5cc6ccccc6c6ccccc56)cc4)c4cccc(-c5cc6ccccc6c6ccccc56)c4)cc32)cc1]

Compound sub2-A-2 (10 g, 34.6 mmol), Compound sub55 (11.5 g, 34.6 mmol), and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.2 g of Compound sub2-B-7. (Yield: 65%, MS: [M+H]⁺=586)

Compound sub2-B-7 (10 g, 17.1 mmol), Compound sub2-A-1 (4.9 g, 17.1 mmol), and sodium tert-butoxide (2.1 g, 22.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 7.7 g of Compound 2-57. (Yield: 54%, MS: [M+H]⁺=838)

### Synthesis Example 2-58

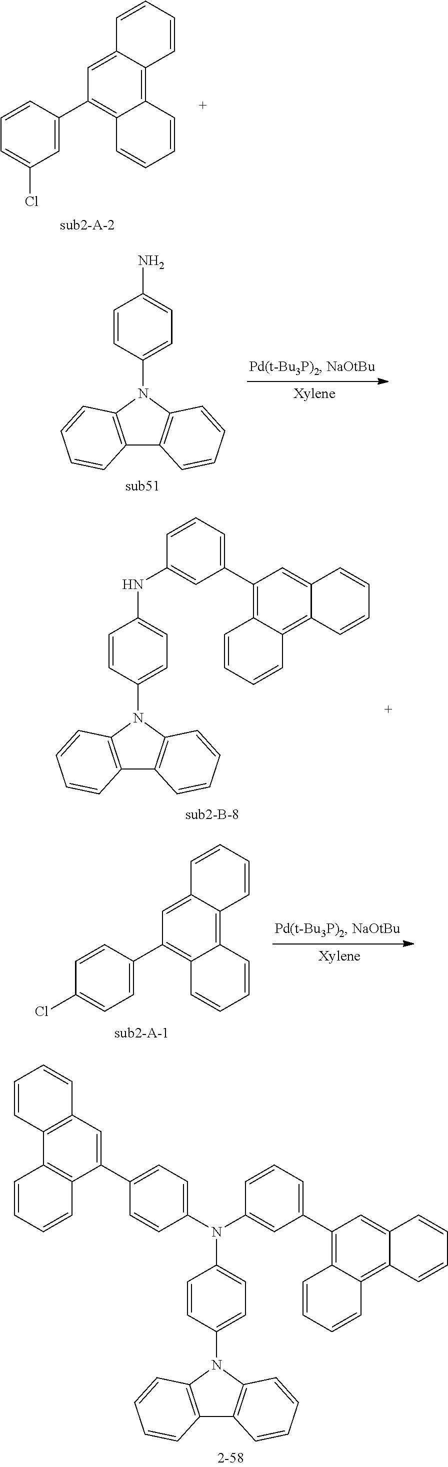 [SMILES: Clc1cccc(-c2cc3ccccc3c3ccccc23)c1 ; Nc1ccc(-n2c3ccccc3c3ccccc32)cc1 ; c1cc(Nc2ccc(-n3c4ccccc4c4ccccc43)cc2)cc(-c2cc3ccccc3c3ccccc23)c1 ; Clc1ccc(-c2cc3ccccc3c3ccccc23)cc1 ; c1cc(-c2cc3ccccc3c3ccccc23)cc(N(c2ccc(-c3cc4ccccc4c4ccccc34)cc2)c2ccc(-n3c4ccccc4c4ccccc43)cc2)c1]

Compound sub2-A-2 (10 g, 34.6 mmol), Compound sub51 (8.9 g, 34.6 mmol), and sodium tert-butoxide (3.7 g, 38.1 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 10.8 g of Compound sub2-B-8. (Yield: 61%, MS: [M+H]⁺=511)

Compound sub2-B-8 (10 g, 19.6 mmol), Compound sub2-A-1 (5.7 g, 19.6 mmol), and sodium tert-butoxide (2.4 g, 25.5 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 7.6 g of Compound 2-58. (Yield: 51%, MS: [M+H]⁺=763)

### Synthesis Example 2-59

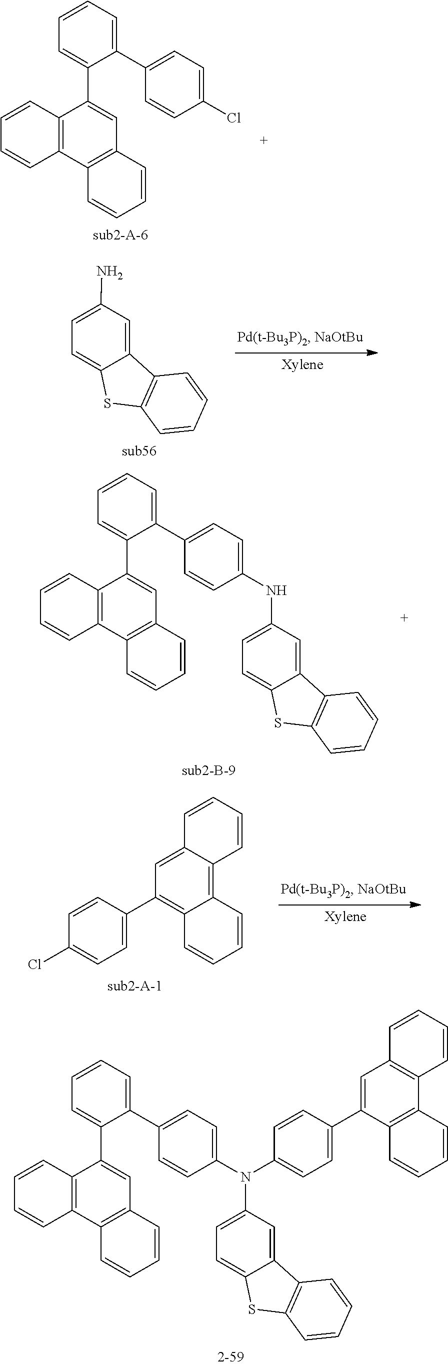 [SMILES: Clc1ccc(-c2ccccc2-c2cc3ccccc3c3ccccc23)cc1 ; Nc1ccc2sc3ccccc3c2c1 ; c1ccc(-c2cc3ccccc3c3ccccc23)c(-c2ccc(Nc3ccc4sc5ccccc5c4c3)cc2)c1 ; Clc1ccc(-c2cc3ccccc3c3ccccc23)cc1 ; c1ccc(-c2cc3ccccc3c3ccccc23)c(-c2ccc(N(c3ccc(-c4cc5ccccc5c5ccccc45)cc3)c3ccc4sc5ccccc5c4c3)cc2)c1]

Compound sub2-A-6 (10 g, 27.4 mmol), Compound sub56 (5.5 g, 27.4 mmol), and sodium tert-butoxide (2.9 g, 30.2 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 7.5 g of Compound sub2-B-9. (Yield: 52%, MS: [M+H]⁺=528)

Compound sub2-B-9 (10 g, 19 mmol), Compound sub2-A-1 (5.5 g, 19 mmol), and sodium tert-butoxide (2.4 g, 24.6 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 8.7 g of Compound 2-59. (Yield: 59%, MS: [M+H]⁺=780)

### Synthesis Example 2-60

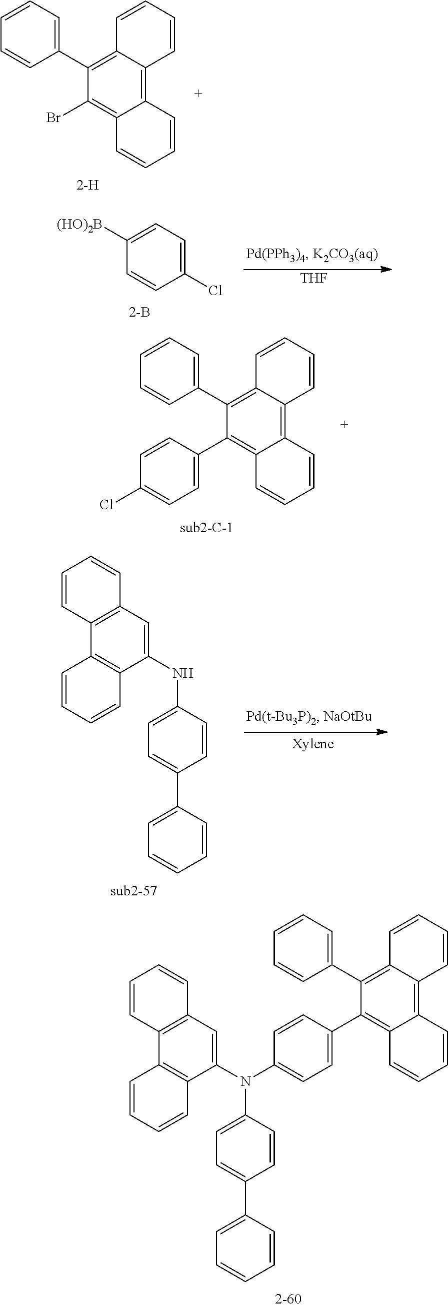 [SMILES: Brc1c(-c2ccccc2)c2ccccc2c2ccccc12 ; OB(O)c1ccc(Cl)cc1 ; Clc1ccc(-c2c(-c3ccccc3)c3ccccc3c3ccccc23)cc1 ; c1ccc(-c2ccc(Nc3cc4ccccc4c4ccccc34)cc2)cc1 ; "THF" ; c1ccc(-c2ccc(N(c3ccc(-c4c(-c5ccccc5)c5ccccc5c5ccccc45)cc3)c3cc4ccccc4c4ccccc34)cc2)cc1]

Compound 2-H (15 g, 45 mmol) and Compound 2-B (7.7 g, 49.5 mmol) were added to 300 ml of THE under a nitrogen atmosphere, and the mixture was stirred and refluxed. Then, potassium carbonate (12.4 g, 90 mmol) was dissolved in 37 ml of water and added thereto, and the mixture was sufficiently stirred and then tetrakis(triphenylphosphine)palladium(0) (1 g, 0.9 mmol) was added. After reacting for 11 hours, the reaction mixture was cooled to room temperature, then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.3 g of Compound sub2-C-1. (Yield: 75%, MS: [M+H]⁺=365)

Compound sub2-C-1 (10 g, 27.4 mmol), Compound sub2-57 (9.5 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.7 g of Compound 2-60. (Yield: 69%, MS: [M+H]⁺=674)

### Synthesis Example 2-61

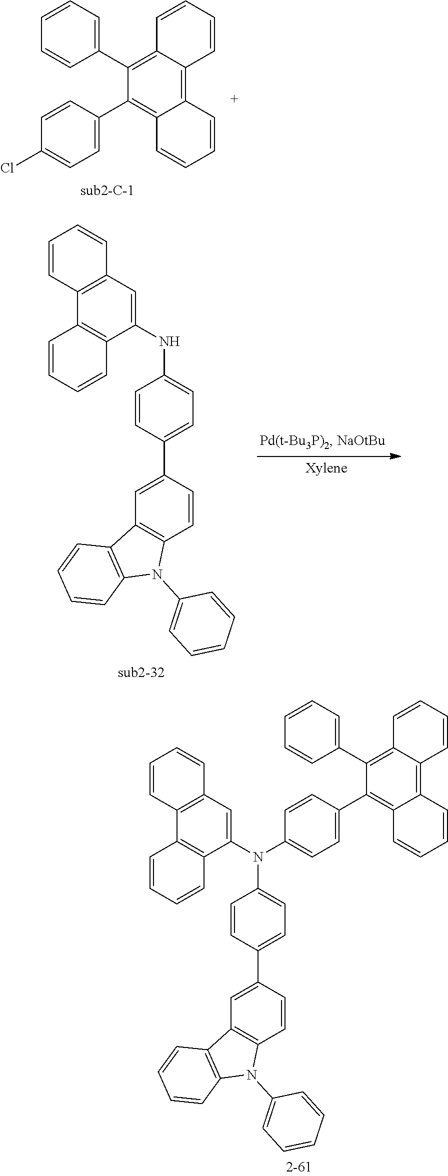 [SMILES: Clc1ccc(-c2c(-c3ccccc3)c3ccccc3c3ccccc23)cc1 ; c1ccc(-n2c3ccccc3c3cc(-c4ccc(Nc5cc6ccccc6c6ccccc56)cc4)ccc32)cc1 ; c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1]

Compound sub2-C-1 (10 g, 27.4 mmol), Compound sub2-32 (14 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.6 g of Compound 2-61. (Yield: 55%, MS: [M+H]⁺=839)

### Synthesis Example 2-62

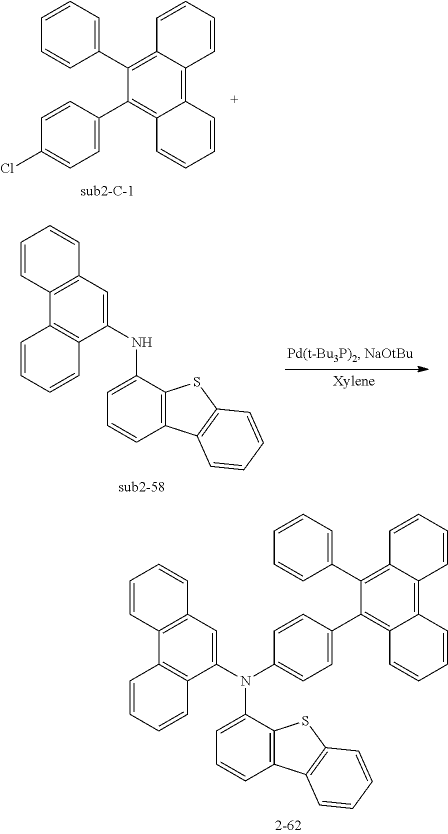 [SMILES: Clc1ccc(-c2c(-c3ccccc3)c3ccccc3c3ccccc23)cc1 ; c1ccc2c(c1)cc(Nc1cccc3c1sc1ccccc13)c1ccccc12 ; c1ccc(-c2c(-c3ccc(N(c4cc5ccccc5c5ccccc45)c4cccc5c4sc4ccccc45)cc3)c3ccccc3c3ccccc23)cc1]

Compound sub2-C-1 (10 g, 27.4 mmol), Compound sub2-58 (10.3 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.5 g of Compound 2-62. (Yield: 65%, MS: [M+H]⁺=704)

### Synthesis Example 2-63

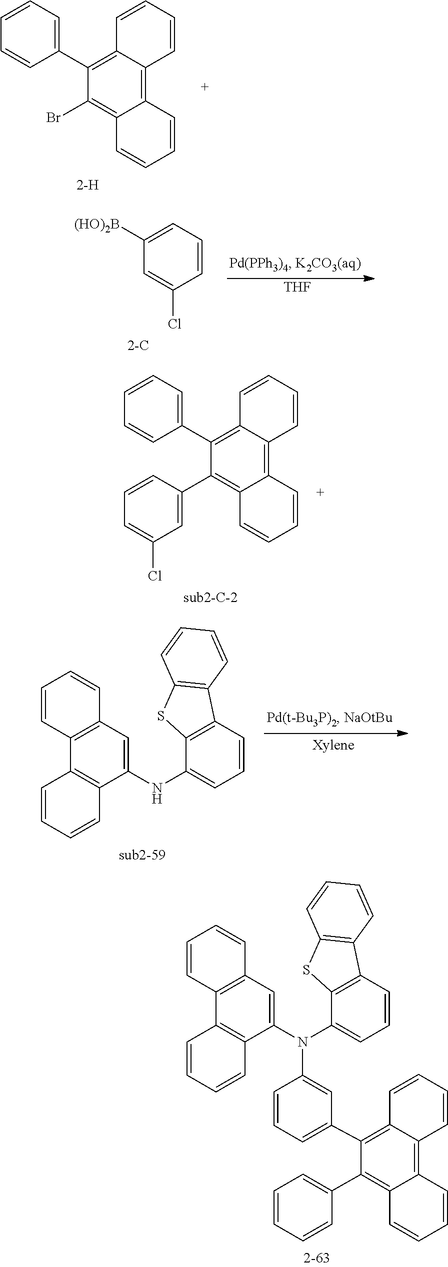 [SMILES: Brc1c(-c2ccccc2)c2ccccc2c2ccccc12 ; OB(O)c1cccc(Cl)c1 ; Clc1cccc(-c2c(-c3ccccc3)c3ccccc3c3ccccc23)c1 ; c1ccc2c(c1)cc(Nc1cccc3c1sc1ccccc13)c1ccccc12 ; "THF" ; c1ccc(-c2c(-c3cccc(N(c4cc5ccccc5c5ccccc45)c4cccc5c4sc4ccccc45)c3)c3ccccc3c3ccccc23)cc1]

Compound 2-H (15 g, 45 mmol) and Compound 2-C (7.7 g, 49.5 mmol) were added to 300 ml of THE under a nitrogen atmosphere, and the mixture was stirred and refluxed. Then, potassium carbonate (12.4 g, 90 mmol) was dissolved in 37 ml of water and added thereto, and the mixture was sufficiently stirred and then tetrakis(triphenylphosphine)palladium(0) (1 g, 0.9 mmol) was added. After reacting for 11 hours, the reaction mixture was cooled to room temperature, then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.3 g of Compound sub2-C-2. (Yield: 75%, MS: [M+H]⁺=365)

Compound sub2-C-2 (10 g, 27.4 mmol), Compound sub2-59 (10.3 g, 27.4 mmol), and sodium tert-butoxide (3.4 g, 35.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 13.5 g of Compound 2-63. (Yield: 70%, MS: [M+H]⁺=704)

### Synthesis Example 2-64

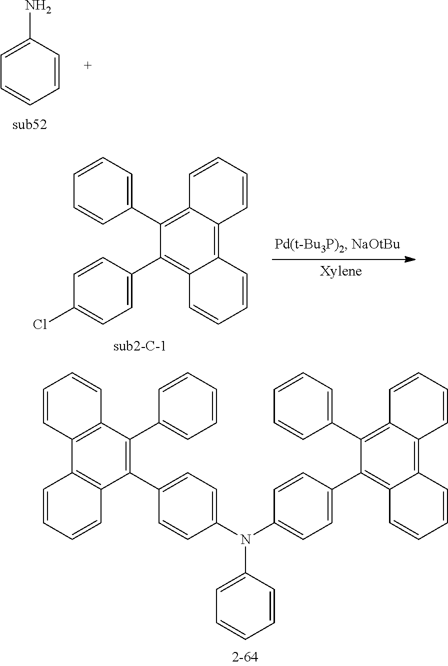 [SMILES: Nc1ccccc1 ; Clc1ccc(-c2c(-c3ccccc3)c3ccccc3c3ccccc23)cc1 ; c1ccc(-c2c(-c3ccc(N(c4ccccc4)c4ccc(-c5c(-c6ccccc6)c6ccccc6c6ccccc56)cc4)cc3)c3ccccc3c3ccccc23)cc1]

Compound sub52 (10 g, 107.4 mmol), Compound sub2-C-1 (82.3 g, 225.5 mmol), and sodium tert-butoxide (25.8 g, 268.4 mmol) were added to 200 ml of xylene under a nitrogen atmosphere, and the mixture was stirred and refluxed. Then, bis(tri-tert-butylphosphine)palladium(0) (1.1 g, 2.1 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 41 g of Compound 2-64. (Yield: 51%, MS: [M+H]⁺=750)

### Synthesis Example 2-65

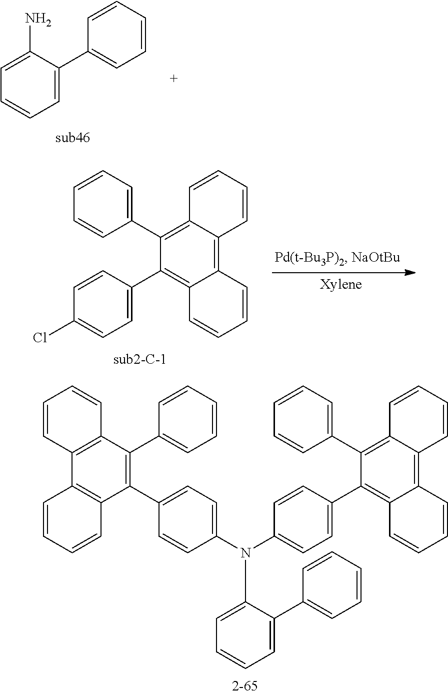 [SMILES: Nc1ccccc1-c1ccccc1 ; Clc1ccc(-c2c(-c3ccccc3)c3ccccc3c3ccccc23)cc1 ; c1ccc(-c2ccccc2N(c2ccc(-c3c(-c4ccccc4)c4ccccc4c4ccccc34)cc2)c2ccc(-c3c(-c4ccccc4)c4ccccc4c4ccccc34)cc2)cc1]

Compound sub46 (10 g, 59.1 mmol), Compound sub2-C-1 (45.3 g, 124.1 mmol), and sodium tert-butoxide (14.2 g, 147.7 mmol) were added to 200 ml of xylene under a nitrogen atmosphere, and the mixture was stirred and refluxed. Then, bis(tri-tert-butylphosphine)palladium(0) (0.6 g, 1.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 31.2 g of Compound 2-65. (Yield: 64%, MS: [M+H]⁺=826)

### Synthesis Example 2-66

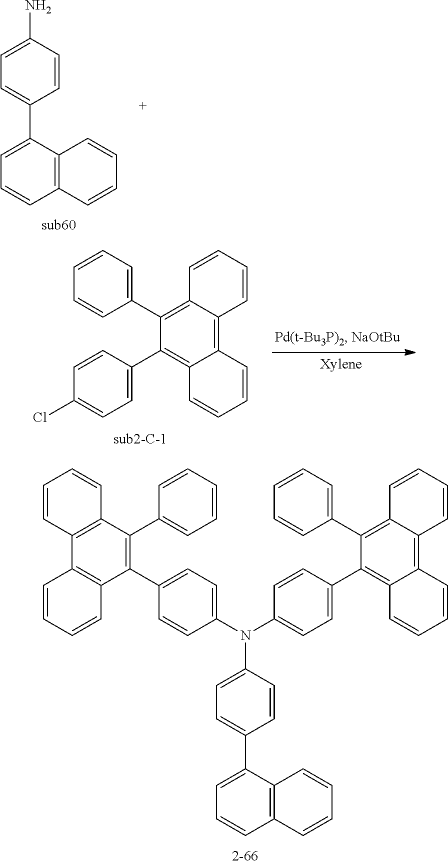 [SMILES: Nc1ccc(-c2cccc3ccccc23)cc1 ; Clc1ccc(-c2c(-c3ccccc3)c3ccccc3c3ccccc23)cc1 ; c1ccc(-c2c(-c3ccc(N(c4ccc(-c5cccc6ccccc56)cc4)c4ccc(-c5c(-c6ccccc6)c6ccccc6c6ccccc56)cc4)cc3)c3ccccc3c3ccccc23)cc1]

Compound sub60 (10 g, 45.6 mmol), Compound sub2-C-1 (34.9 g, 95.8 mmol), and sodium tert-butoxide (11 g, 114 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 26.7 g of Compound 2-66. (Yield: 67%, MS: [M+H]⁺=876)

### Synthesis Example 2-67

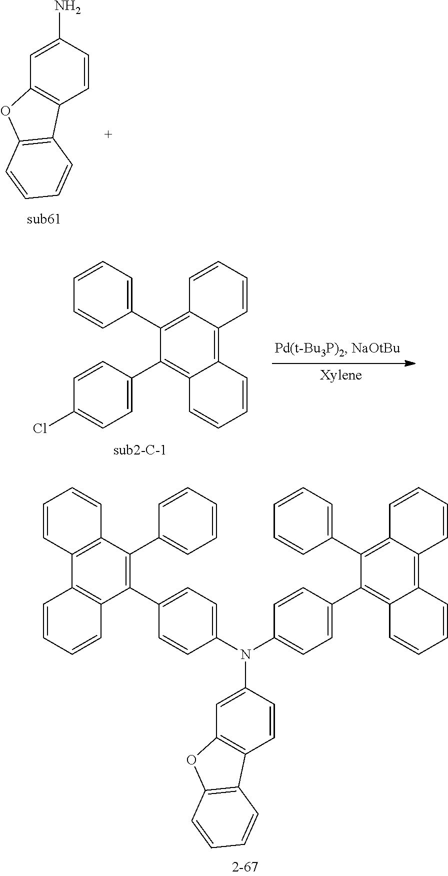 [SMILES: Nc1ccc2c(c1)oc1ccccc12 ; Clc1ccc(-c2c(-c3ccccc3)c3ccccc3c3ccccc23)cc1 ; c1ccc(-c2c(-c3ccc(N(c4ccc(-c5c(-c6ccccc6)c6ccccc6c6ccccc56)cc4)c4ccc5c(c4)oc4ccccc45)cc3)c3ccccc3c3ccccc23)cc1]

Compound sub61 (10 g, 54.6 mmol), Compound sub2-C-1 (41.8 g, 114.6 mmol), and sodium tert-butoxide (13.1 g, 136.5 mmol) were added to 200 ml of xylene under a nitrogen atmosphere, and the mixture was stirred and refluxed. Then, bis(tri-tert-butylphosphine)palladium(0) (0.6 g, 1.1 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 32.1 g of Compound 2-67. (Yield: 70%, MS: [M+H]⁺=840)

### Synthesis Example 2-68

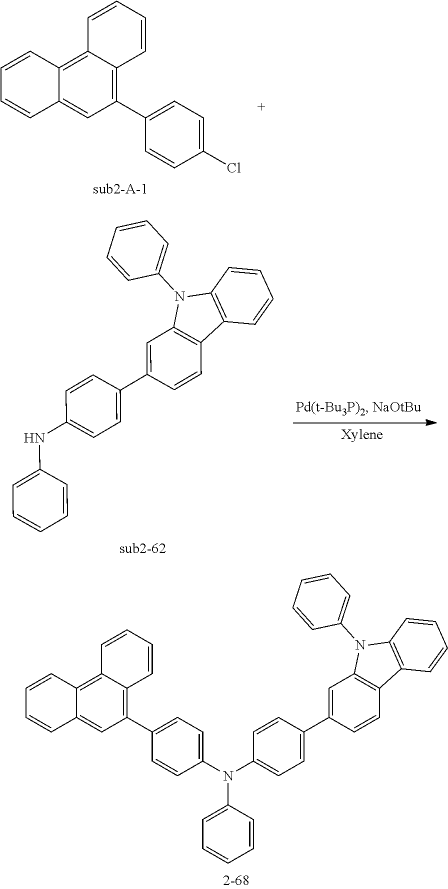 [SMILES: Clc1ccc(-c2cc3ccccc3c3ccccc23)cc1 ; c1ccc(Nc2ccc(-c3ccc4c5ccccc5n(-c5ccccc5)c4c3)cc2)cc1 ; c1ccc(N(c2ccc(-c3ccc4c5ccccc5n(-c5ccccc5)c4c3)cc2)c2ccc(-c3cc4ccccc4c4ccccc34)cc2)cc1]

Compound sub2-A-1 (10 g, 34.6 mmol), Compound sub2-62 (15.6 g, 38.1 mmol), and sodium tert-butoxide (4.3 g, 45.01 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.6 g of Compound 2-68. (Yield: 55%, MS: [M+H]⁺=663)

### Synthesis Example 2-69

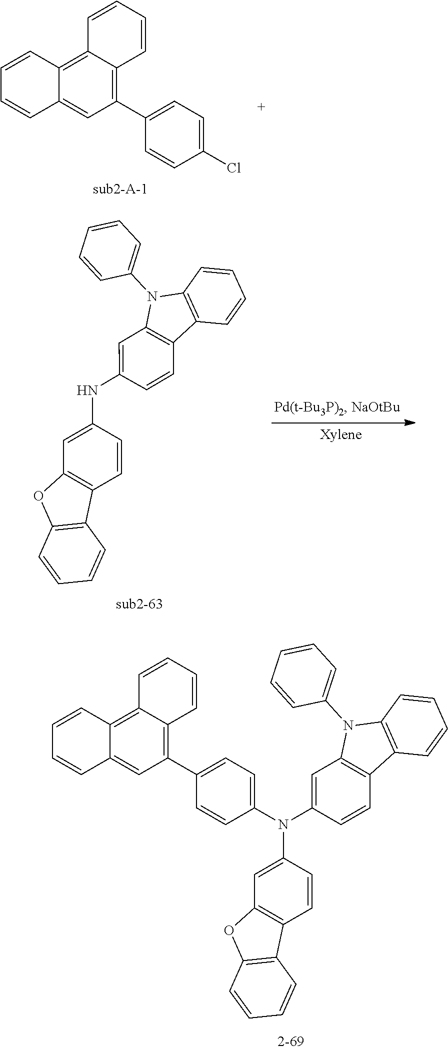 [SMILES: Clc1ccc(-c2cc3ccccc3c3ccccc23)cc1 ; c1ccc(-n2c3ccccc3c3ccc(Nc4ccc5c(c4)oc4ccccc45)cc32)cc1 ; c1ccc(-n2c3ccccc3c3ccc(N(c4ccc(-c5cc6ccccc6c6ccccc56)cc4)c4ccc5c(c4)oc4ccccc45)cc32)cc1]

Compound sub2-A-1 (10 g, 34.6 mmol), Compound sub2-63 (16.2 g, 38.1 mmol), and sodium tert-butoxide (4.8 g, 19.5 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 12.6 g of Compound 2-69. (Yield: 54%, MS: [M+H]⁺=677)

### Synthesis Example 2-70

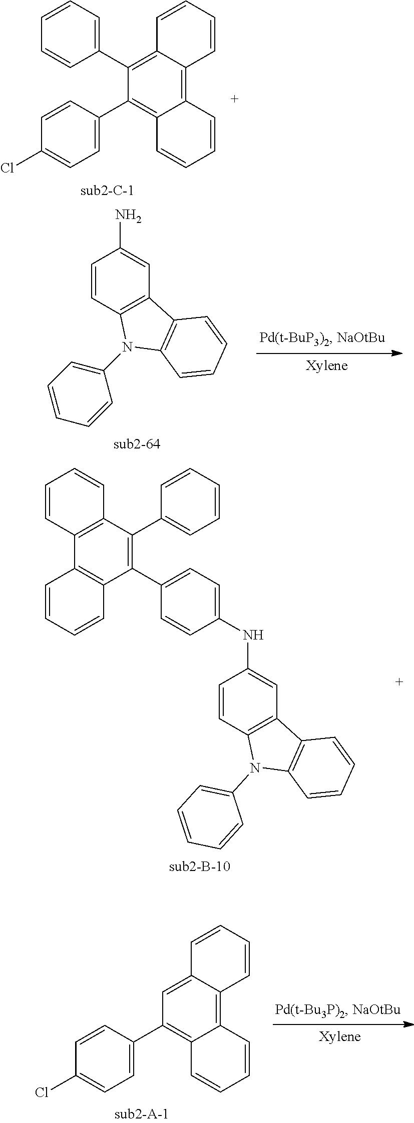

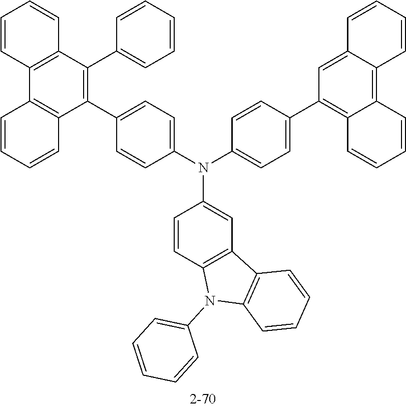 [SMILES: c1ccc(-c2c(-c3ccc(N(c4ccc(-c5cc6ccccc6c6ccccc56)cc4)c4ccc5c(c4)c4ccccc4n5-c4ccccc4)cc3)c3ccccc3c3ccccc23)cc1]

Compound sub2-C-1 (10 g, 27.4 mmol), Compound sub2-64 (7.8 g, 30.1 mmol), and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 11.2 g of Compound sub2-B-10. (Yield: 70%, MS: [M+H]⁺=587)

Compound sub2-B-10 (10 g, 17 mmol), Compound sub2-A-1 (5.4 g, 18.7 mmol), and sodium tert-butoxide (2.4 g, 24.4 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 8.7 g of Compound 2-70. (Yield: 61%, MS: [M+H]⁺=839)

### Example 1

A glass substrate on which a thin film of ITO (indium tin oxide) was coated in a thickness of 1000 Å was put into distilled water containing a detergent dissolved therein and ultrasonically washed. In this case, the detergent used was a product commercially available from Fischer Co. and the distilled water was one which had been twice filtered by using a filter commercially available from Millipore Co. The ITO was cleaned for 30 minutes, and ultrasonic cleaning was then repeated twice for 10 minutes by using distilled water. After the cleaning with distilled water was completed, the substrate was ultrasonically washed with the solvents of isopropyl alcohol, acetone, and methanol, and dried, after which it was transported to a plasma cleaner. Then, the substrate was cleaned with oxygen plasma for 5 minutes, and then transferred to a vacuum evaporator.

On the ITO transparent electrode thus prepared, the following compound HI-1 was formed to a thickness of 1150 Å as a hole injection layer, but the following compound A-1 was p-doped at a concentration of 1.5 wt. %. The following compound HT-1 was vacuum deposited on the hole injection layer to form a hole transport layer with a layer thickness of 800 Å. Then, the following compound EB-1 was vacuum deposited on the hole transport layer to a layer thickness of 150 Å to form an electron blocking layer. Then, Compound 1-1 and Compound 2-1 as the host and the following Compound Dp-7 as the dopant were vacuum deposited in a weight ratio of 49:49:2 on the EB-1 deposited layer to form a red light emitting layer with a layer thickness of 400 Å. The following compound HB-1 was vacuum deposited on the light emitting layer to a layer thickness of 30 Å to form a hole blocking layer. The following compound ET-1 and the following compound LiQ were vacuum deposited in a weight ratio of 2:1 on the hole blocking layer to form an electron injection and transport layer with a layer thickness of 300 Å. Lithium fluoride (LiF) and aluminum were sequentially deposited to have a thickness of 12 Å and 1,000 Å, respectively, on the electron injection and transport layer, thereby forming a cathode.

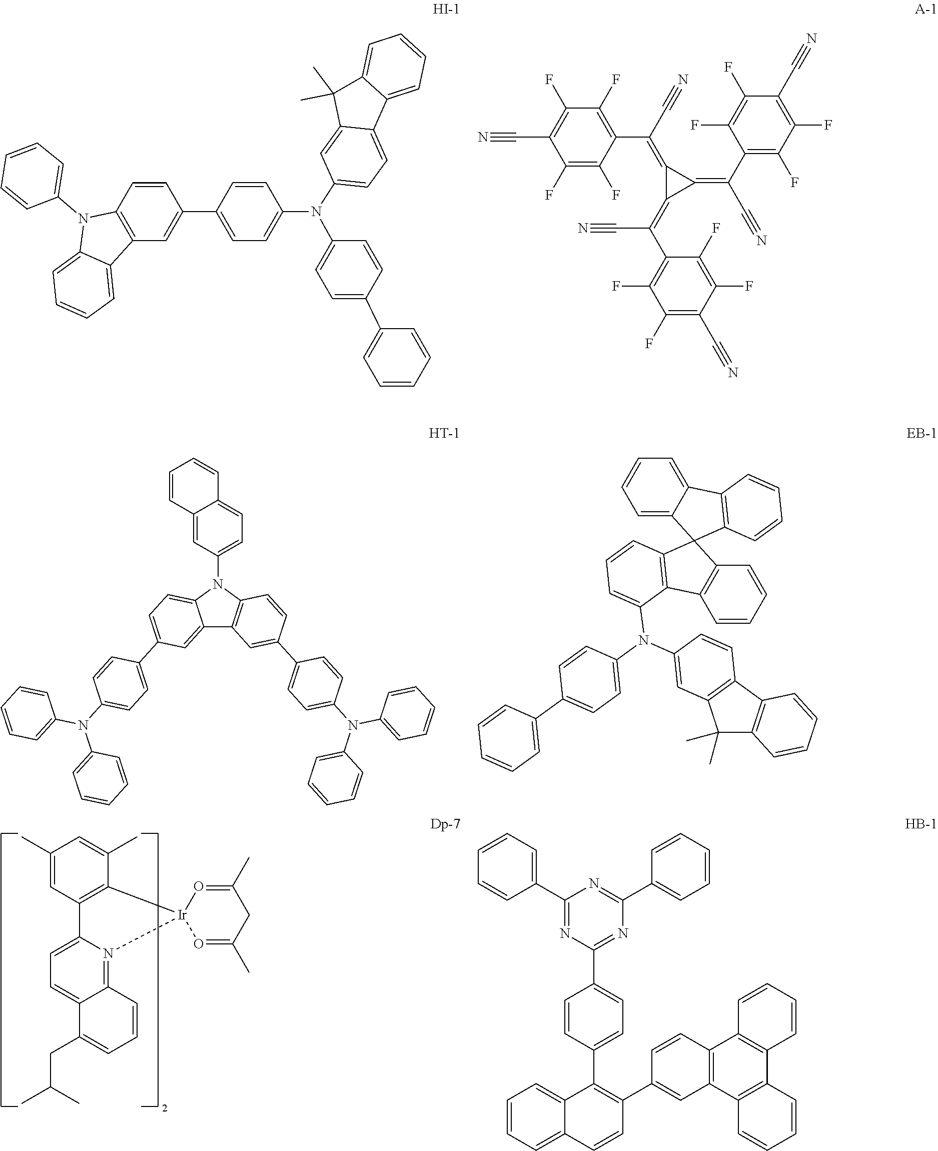

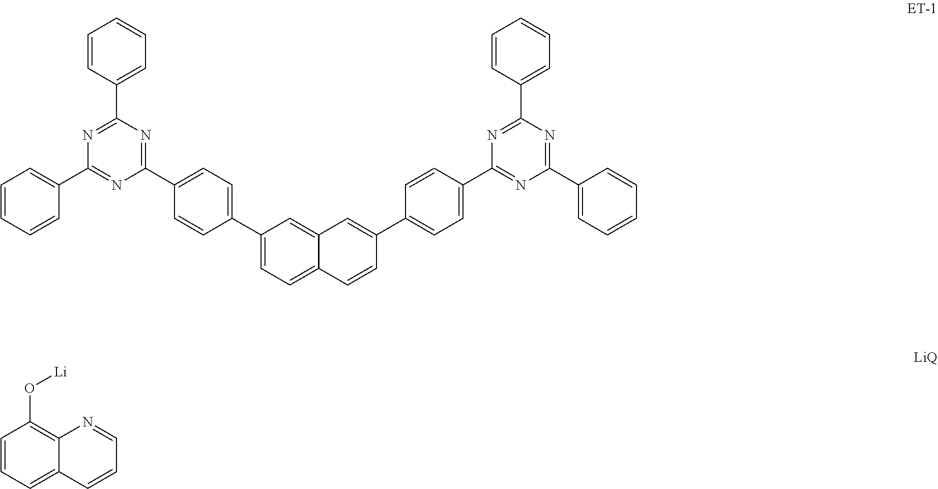

In the above-mentioned processes, the deposition rates of the organic materials were maintained at 0.4˜0.7 Å/sec, the deposition rates of lithium fluoride and aluminum of the cathode were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

### Examples 2 to 220

The organic light emitting devices were manufactured in the same manner as in Example 1, except that in the organic light emitting device of Example 1, the first host and the second host described in Tables 1 to 5 below were used instead of Compound 1-1 and Compound 2-1.

### Comparative Examples 1 to 60

The organic light emitting devices were manufactured in the same manner as in Example 1, except that in the organic light emitting device of Example 1, the first host described in Tables 6 and 7 below were used instead of Compound 1-1. The structures of Compounds B-1 to B-12 of Tables 6 and 7 are as follows.

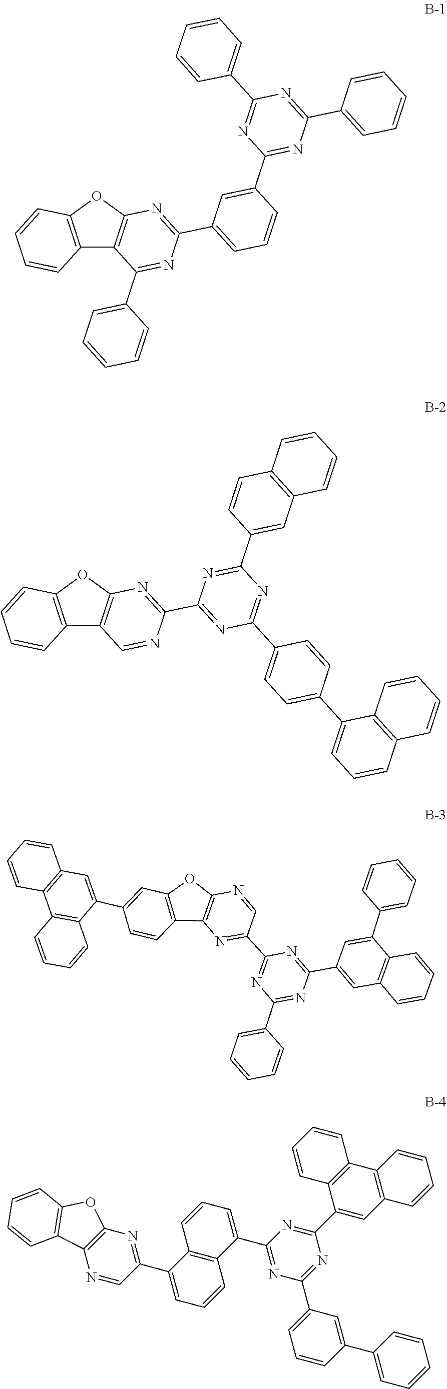 [SMILES: c1ccc(-c2nc(-c3ccccc3)nc(-c3cccc(-c4nc(-c5ccccc5)c5c(n4)oc4ccccc45)c3)n2)cc1 ; c1ccc2cc(-c3nc(-c4ccc(-c5cccc6ccccc56)cc4)nc(-c4ncc5c(n4)oc4ccccc45)n3)ccc2c1 ; c1ccc(-c2nc(-c3cc(-c4ccccc4)c4ccccc4c3)nc(-c3cnc4oc5cc(-c6cc7ccccc7c7ccccc67)ccc5c4n3)n2)cc1 ; c1ccc(-c2cccc(-c3nc(-c4cccc5c(-c6cnc7c(n6)oc6ccccc67)cccc45)nc(-c4cc5ccccc5c5ccccc45)n3)c2)cc1]

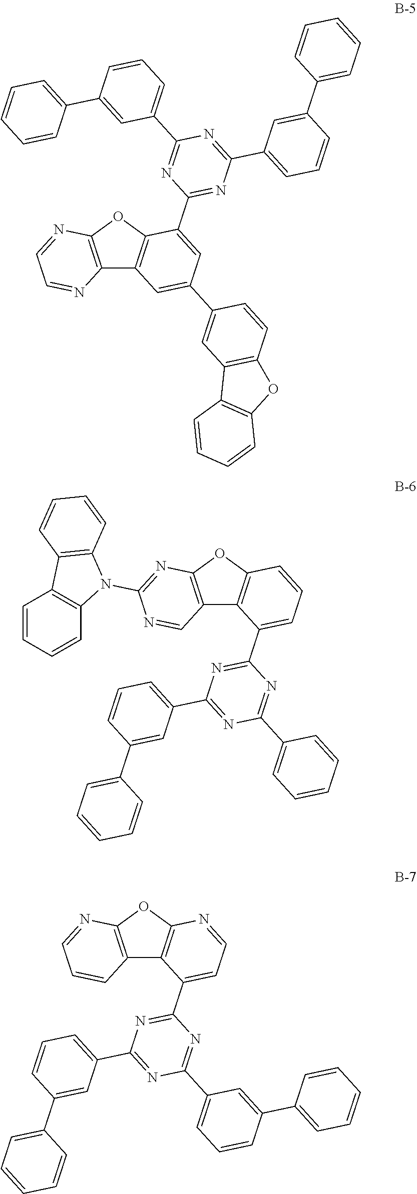 [SMILES: c1ccc(-c2cccc(-c3nc(-c4cccc(-c5ccccc5)c4)nc(-c4cc(-c5ccc6oc7ccccc7c6c5)cc5c4oc4nccnc45)n3)c2)cc1 ; c1ccc(-c2cccc(-c3nc(-c4ccccc4)nc(-c4cccc5oc6nc(-n7c8ccccc8c8ccccc87)ncc6c45)n3)c2)cc1 ; c1ccc(-c2cccc(-c3nc(-c4cccc(-c5ccccc5)c4)nc(-c4ccnc5oc6ncccc6c45)n3)c2)cc1]

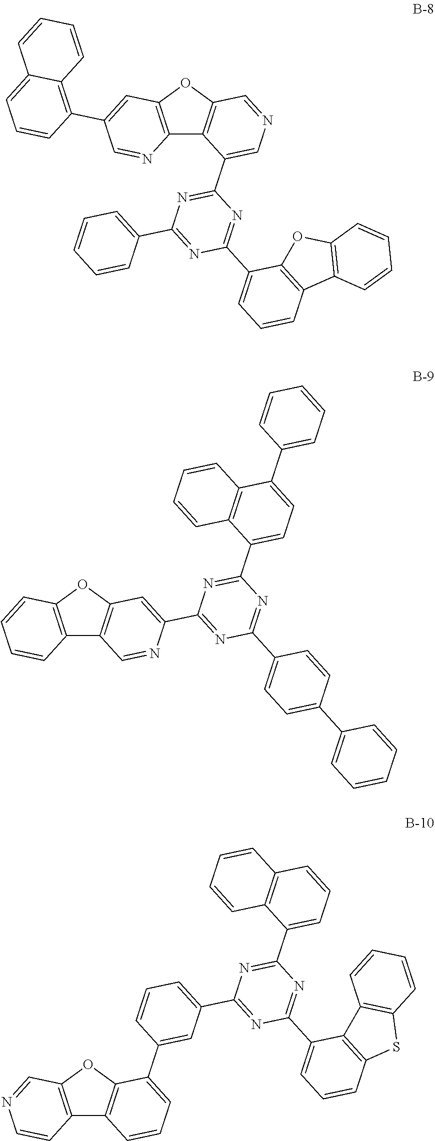 [SMILES: c1ccc(-c2nc(-c3cccc4c3oc3ccccc34)nc(-c3cncc4oc5cc(-c6cccc7ccccc67)cnc5c34)n2)cc1 ; c1ccc(-c2ccc(-c3nc(-c4cc5oc6ccccc6c5cn4)nc(-c4ccc(-c5ccccc5)c5ccccc45)n3)cc2)cc1 ; c1cc(-c2nc(-c3cccc4ccccc34)nc(-c3cccc4sc5ccccc5c34)n2)cc(-c2cccc3c2oc2cnccc23)c1]

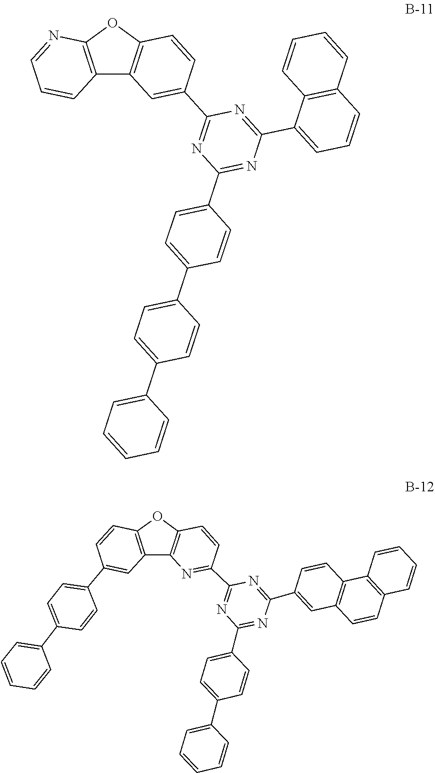 [SMILES: c1ccc(-c2ccc(-c3ccc(-c4nc(-c5ccc6oc7ncccc7c6c5)nc(-c5cccc6ccccc56)n4)cc3)cc2)cc1 ; c1ccc(-c2ccc(-c3ccc4oc5ccc(-c6nc(-c7ccc(-c8ccccc8)cc7)nc(-c7ccc8c(ccc9ccccc98)c7)n6)nc5c4c3)cc2)cc1]

### Comparative Examples 61 to 156

The organic light emitting devices were manufactured in the same manner as in Example 1, except that in the organic light emitting device of Example 1, the second host described in Tables 8 to 10 below were used instead of Compound 2-1. The structures of Compounds C-1 to C-12 of Tables 8 to 10 are as follows.

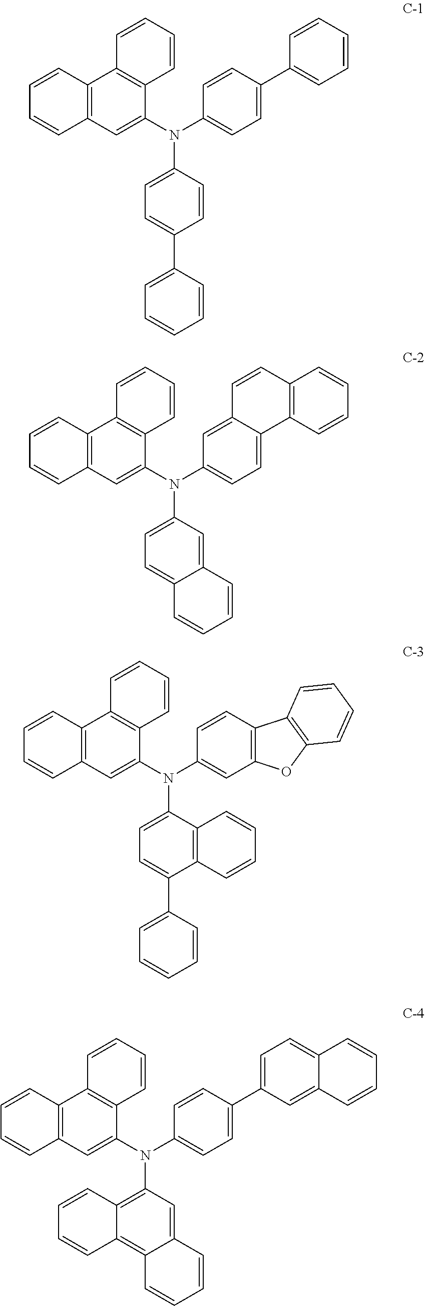

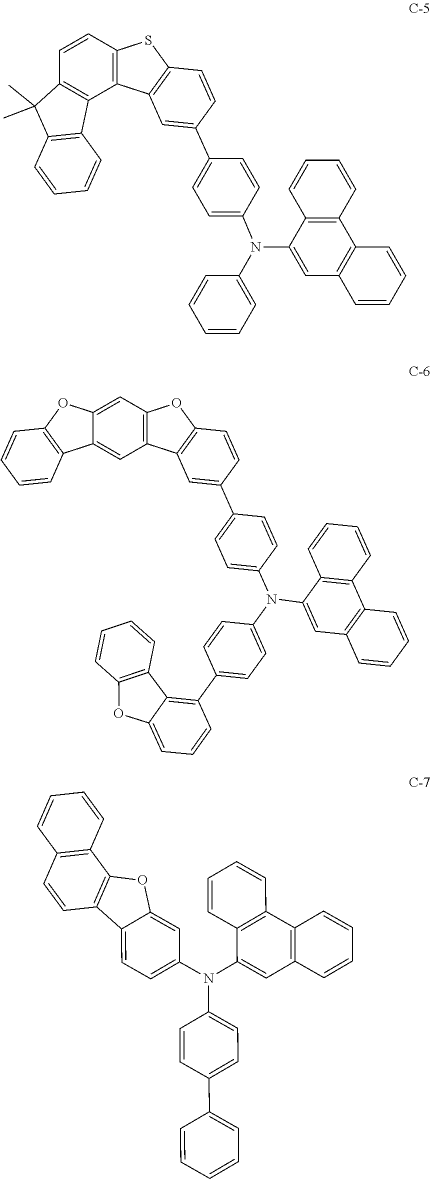 [SMILES: CC1(C)c2ccccc2-c2c1ccc1sc3ccc(-c4ccc(N(c5ccccc5)c5cc6ccccc6c6ccccc56)cc4)cc3c21 ; c1ccc2c(c1)cc(N(c1ccc(-c3ccc4oc5cc6oc7ccccc7c6cc5c4c3)cc1)c1ccc(-c3cccc4oc5ccccc5c34)cc1)c1ccccc12 ; c1ccc(-c2ccc(N(c3ccc4c(c3)oc3c5ccccc5ccc43)c3cc4ccccc4c4ccccc34)cc2)cc1]

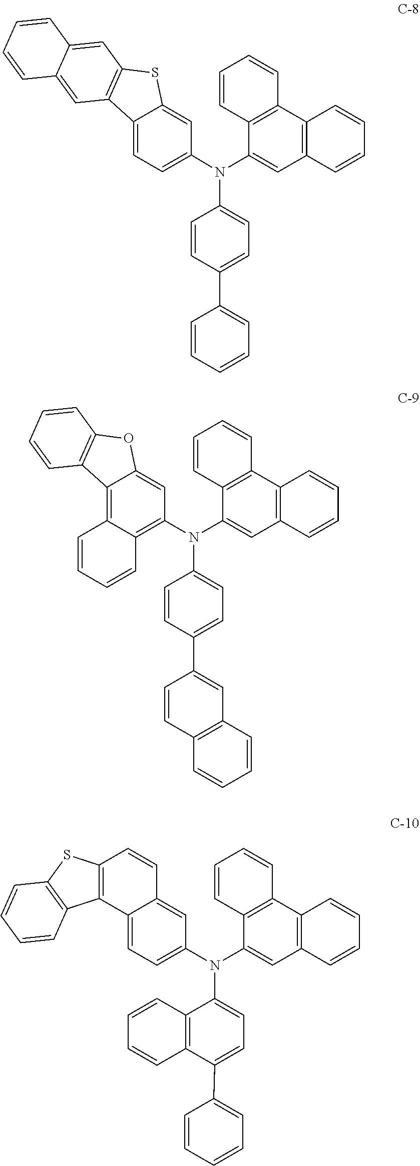 [SMILES: c1ccc(-c2ccc(N(c3ccc4c(c3)sc3cc5ccccc5cc34)c3cc4ccccc4c4ccccc34)cc2)cc1 ; c1ccc2cc(-c3ccc(N(c4cc5ccccc5c5ccccc45)c4cc5oc6ccccc6c5c5ccccc45)cc3)ccc2c1 ; c1ccc(-c2ccc(N(c3ccc4c(ccc5sc6ccccc6c54)c3)c3cc4ccccc4c4ccccc34)c3ccccc23)cc1]

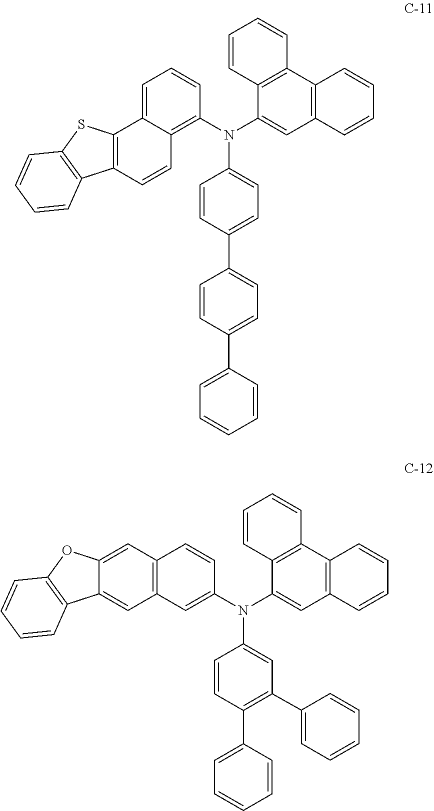 [SMILES: c1ccc(-c2ccc(-c3ccc(N(c4cc5ccccc5c5ccccc45)c4cccc5c4ccc4c6ccccc6sc54)cc3)cc2)cc1 ; c1ccc(-c2ccc(N(c3ccc4cc5oc6ccccc6c5cc4c3)c3cc4ccccc4c4ccccc34)cc2-c2ccccc2)cc1]

### Experimental Example

The voltage and efficiency were measured (based on 15 mA/cm²) by applying a current to the organic light emitting devices manufactured in Examples 1 to 220 and Comparative Examples 1 to 156, and the results are shown in Tables 1 to 4 below. Lifetime T95 means the time required for the luminance to be reduced to 95% of the initial luminance (6,000 nit).

TABLE 1

| | | | | Effi- | | |
|---|---|---|---|---|---|---|
| | First | Second | Voltage | ciency | Lifetime | |
| | Host | Host | (V) | (cd/A) | T95(hr) | Color |
| Example 1 | Compound | Compound | 3.59 | 21.67 | 237 | Red |
| | 1-1 | 2-1 | | | | |
| Example 2 | | Compound | 3.61 | 21.20 | 215 | Red |
| | | 2-27 | | | | |
| Example 3 | | Compound | 3.61 | 21.88 | 238 | Red |
| | | 2-39 | | | | |
| Example 4 | | Compound | 3.54 | 21.58 | 238 | Red |
| | | 2-54 | | | | |
| Example 5 | | Compound | 3.52 | 21.43 | 241 | Red |
| | | 2-60 | | | | |
| Example 6 | Compound | Compound | 3.54 | 21.82 | 214 | Red |
| | 1-2 | 2-3 | | | | |
| Example 7 | | Compound | 3.67 | 21.79 | 236 | Red |
| | | 2-10 | | | | |
| Example 8 | | Compound | 3.55 | 21.43 | 220 | Red |
| | | 2-68 | | | | |
| Example 9 | | Compound | 3.64 | 21.48 | 225 | Red |
| | | 2-44 | | | | |
| Example 10 | | Compound | 3.66 | 21.72 | 227 | Red |
| | | 2-49 | | | | |
| Example 11 | Compound | Compound | 3.53 | 22.37 | 258 | Red |
| | 1-4 | 2-5 | | | | |
| Example 12 | | Compound | 3.49 | 22.61 | 252 | Red |
| | | 2-14 | | | | |
| Example 13 | | Compound | 3.54 | 22.17 | 260 | Red |
| | | 2-23 | | | | |
| Example 14 | | Compound | 3.54 | 22.55 | 254 | Red |
| | | 2-58 | | | | |
| Example 15 | | Compound | 3.48 | 23.00 | 259 | Red |
| | | 2-64 | | | | |
| Example 16 | Compound | Compound | 3.52 | 23.07 | 263 | Red |
| | 1-5 | 2-17 | | | | |
| Example 17 | | Compound | 3.52 | 22.22 | 254 | Red |
| | | 2-20 | | | | |
| Example 18 | | Compound | 3.55 | 22.88 | 232 | Red |
| | | 2-28 | | | | |
| Example 19 | | Compound | 3.52 | 22.24 | 246 | Red |
| | | 2-35 | | | | |
| Example 20 | | Compound | 3.48 | 23.20 | 262 | Red |
| | | 2-57 | | | | |
| Example 21 | Compound | Compound | 3.65 | 21.75 | 231 | Red |
| | 1-6 | 2-1 | | | | |
| Example 22 | | Compound | 3.52 | 21.33 | 237 | Red |
| | | 2-27 | | | | |
| Example 23 | | Compound | 3.65 | 21.34 | 224 | Red |
| | | 2-39 | | | | |
| Example 24 | | Compound | 3.53 | 21.74 | 234 | Red |
| | | 2-54 | | | | |
| Example 25 | | Compound | 3.64 | 21.97 | 219 | Red |
| | | 2-60 | | | | |
| Example 26 | Compound | Compound | 3.65 | 21.52 | 231 | Red |
| | 1-7 | 2-3 | | | | |
| Example 27 | | Compound | 3.58 | 21.87 | 247 | Red |
| | | 2-10 | | | | |
| Example 28 | | Compound | 3.59 | 21.35 | 234 | Red |
| | | 2-68 | | | | |
| Example 29 | | Compound | 3.56 | 22.30 | 218 | Red |
| | | 2-44 | | | | |
| Example 30 | | Compound | 3.59 | 22.18 | 244 | Red |
| | | 2-49 | | | | |
| Example 31 | Compound | Compound | 3.45 | 22.18 | 263 | Red |
| | 1-9 | 2-5 | | | | |
| Example 32 | | Compound | 3.41 | 22.23 | 279 | Red |
| | | 2-14 | | | | |
| Example 33 | | Compound | 3.38 | 22.21 | 281 | Red |
| | | 2-23 | | | | |
| Example 34 | | Compound | 3.37 | 22.15 | 260 | Red |
| | | 2-58 | | | | |
| Example 35 | | Compound | 3.38 | 21.77 | 277 | Red |
| | | 2-64 | | | | |
| Example 36 | Compound | Compound | 3.47 | 22.16 | 255 | Red |
| | 1-11 | 2-17 | | | | |
| Example 37 | | Compound | 3.37 | 21.21 | 262 | Red |
| | | 2-20 | | | | |
| Example 38 | | Compound | 3.37 | 22.23 | 259 | Red |
| | | 2-28 | | | | |
| Example 39 | | Compound | 3.45 | 22.27 | 277 | Red |
| | | 2-35 | | | | |
| Example 40 | | Compound | 3.41 | 21.94 | 254 | Red |
| | | 2-70 | | | | |
| Example 41 | Compound | Compound | 3.44 | 23.03 | 252 | Red |
| | 1-12 | 2-1 | | | | |
| Example 42 | | Compound | 3.50 | 22.87 | 257 | Red |
| | | 2-27 | | | | |
| Example 43 | | Compound | 3.45 | 22.80 | 254 | Red |
| | | 2-39 | | | | |
| Example 44 | | Compound | 3.51 | 22.15 | 261 | Red |
| | | 2-54 | | | | |
| Example 45 | | Compound | 3.53 | 22.63 | 248 | Red |
| | | 2-60 | | | | |

TABLE 2

| | | | | Effi- | | |
|---|---|---|---|---|---|---|
| | First | Second | Voltage | ciency | Lifetime | |
| | Host | Host | (V) | (cd/A) | T95(hr) | Color |
| Example 46 | Compound | Compound | 3.51 | 22.65 | 263 | Red |
| | 1-14 | 2-3 | | | | |
| Example 47 | | Compound | 3.48 | 22.55 | 242 | Red |
| | | 2-10 | | | | |
| Example 48 | | Compound | 3.51 | 22.33 | 254 | Red |
| | | 2-68 | | | | |
| Example 49 | | Compound | 3.54 | 22.23 | 232 | Red |
| | | 2-44 | | | | |
| Example 50 | | Compound | 3.54 | 22.13 | 257 | Red |
| | | 2-49 | | | | |
| Example 51 | Compound | Compound | 3.45 | 21.81 | 281 | Red |
| | 1-15 | 2-5 | | | | |
| Example 52 | | Compound | 3.42 | 22.14 | 280 | Red |
| | | 2-14 | | | | |
| Example 53 | | Compound | 3.42 | 22.23 | 267 | Red |
| | | 2-23 | | | | |
| Example 54 | | Compound | 3.44 | 21.45 | 255 | Red |
| | | 2-58 | | | | |
| Example 55 | | Compound | 3.35 | 21.33 | 254 | Red |
| | | 2-64 | | | | |
| Example 56 | Compound | Compound | 3.62 | 21.24 | 208 | Red |
| | 1-17 | 2-17 | | | | |
| Example 57 | | Compound | 3.65 | 21.85 | 228 | Red |
| | | 2-20 | | | | |
| Example 58 | | Compound | 3.57 | 22.09 | 232 | Red |
| | | 2-28 | | | | |
| Example 59 | | Compound | 3.52 | 21.29 | 232 | Red |
| | | 2-35 | | | | |
| Example 60 | | Compound | 3.65 | 21.60 | 206 | Red |
| | | 2-57 | | | | |
| Example 61 | Compound | Compound | 3.66 | 21.41 | 221 | Red |
| | 1-18 | 2-1 | | | | |
| Example 62 | | Compound | 3.55 | 21.38 | 218 | Red |
| | | 2-27 | | | | |
| Example 63 | | Compound | 3.62 | 21.54 | 229 | Red |
| | | 2-39 | | | | |
| Example 64 | | Compound | 3.55 | 22.13 | 219 | Red |
| | | 2-54 | | | | |
| Example 65 | | Compound | 3.64 | 21.34 | 225 | Red |
| | | 2-60 | | | | |
| Example 66 | Compound | Compound | 3.88 | 19.7 | 227 | Red |
| | 1-19 | 2-3 | | | | |
| Example 67 | | Compound | 3.84 | 20.8 | 218 | Red |
| | | 2-10 | | | | |
| Example 68 | | Compound | 3.78 | 19.3 | 215 | Red |
| | | 2-68 | | | | |
| Example 69 | | Compound | 3.82 | 19.5 | 220 | Red |
| | | 2-44 | | | | |
| Example 70 | | Compound | 3.86 | 20.2 | 218 | Red |
| | | 2-49 | | | | |
| Example 71 | Compound | Compound | 3.98 | 20.3 | 256 | Red |
| | 1-23 | 2-5 | | | | |
| Example 72 | | Compound | 3.92 | 20.0 | 248 | Red |
| | | 2-14 | | | | |
| Example 73 | | Compound | 3.95 | 20.5 | 251 | Red |
| | | 2-23 | | | | |
| Example 74 | | Compound | 3.90 | 20.1 | 252 | Red |
| | | 2-58 | | | | |
| Example 75 | | Compound | 3.93 | 19.8 | 265 | Red |
| | | 2-64 | | | | |
| Example 76 | Compound | Compound | 3.60 | 21.33 | 223 | Red |
| | 1-24 | 2-1 | | | | |
| Example 77 | | Compound | 3.58 | 21.44 | 223 | Red |
| | | 2-27 | | | | |
| Example 78 | | Compound | 3.61 | 22.16 | 209 | Red |
| | | 2-39 | | | | |
| Example 79 | | Compound | 3.56 | 21.83 | 224 | Red |
| | | 2-54 | | | | |
| Example 80 | | Compound | 3.59 | 21.35 | 234 | Red |
| | | 2-60 | | | | |
| Example 81 | Compound | Compound | 3.61 | 21.90 | 215 | Red |
| | 1-25 | 2-17 | | | | |
| Example 82 | | Compound | 3.54 | 22.23 | 218 | Red |
| | | 2-20 | | | | |
| Example 83 | | Compound | 3.52 | 21.61 | 217 | Red |
| | | 2-28 | | | | |
| Example 84 | | Compound | 3.63 | 21.55 | 209 | Red |
| | | 2-35 | | | | |
| Example 85 | | Compound | 3.64 | 21.29 | 220 | Red |
| | | 2-70 | | | | |
| Example 86 | Compound | Compound | 3.67 | 21.52 | 223 | Red |
| | 1-26 | 2-3 | | | | |
| Example 87 | | Compound | 3.62 | 21.60 | 223 | Red |
| | | 2-10 | | | | |
| Example 88 | | Compound | 3.60 | 21.29 | 209 | Red |
| | | 2-68 | | | | |
| Example 89 | | Compound | 3.63 | 21.39 | 224 | Red |
| | | 2-44 | | | | |
| Example 90 | | Compound | 3.73 | 20.87 | 234 | Red |
| | | 2-49 | | | | |

TABLE 3

| | | | | Effi- | | |
|---|---|---|---|---|---|---|
| | First | Second | Voltage | ciency | Lifetime | |
| | Host | Host | (V) | (cd/A) | T95(hr) | Color |
| Example 91 | Compound | Compound | 3.66 | 20.84 | 215 | Red |
| | 1-27 | 2-5 | | | | |
| Example 92 | | Compound | 3.66 | 21.49 | 218 | Red |
| | | 2-14 | | | | |
| Example 93 | | Compound | 3.61 | 21.46 | 217 | Red |
| | | 2-23 | | | | |
| Example 94 | | Compound | 3.60 | 21.34 | 209 | Red |
| | | 2-58 | | | | |
| Example 95 | | Compound | 3.66 | 21.43 | 220 | Red |
| | | 2-64 | | | | |
| Example 96 | Compound | Compound | 3.59 | 21.76 | 211 | Red |
| | 1-29 | 2-17 | | | | |
| Example 97 | | Compound | 3.61 | 21.73 | 231 | Red |
| | | 2-20 | | | | |
| Example 98 | | Compound | 3.65 | 21.85 | 231 | Red |
| | | 2-28 | | | | |
| Example 99 | | Compound | 3.64 | 21.41 | 214 | Red |
| | | 2-35 | | | | |
| Example 100 | | Compound | 3.64 | 21.78 | 214 | Red |
| | | 2-57 | | | | |
| Example 101 | Compound | Compound | 3.71 | 21.17 | 209 | Red |
| | 1-31 | 2-1 | | | | |
| Example 102 | | Compound | 3.69 | 21.55 | 233 | Red |
| | | 2-27 | | | | |
| Example 103 | | Compound | 3.70 | 21.38 | 205 | Red |
| | | 2-39 | | | | |
| Example 104 | | Compound | 3.66 | 21.18 | 225 | Red |
| | | 2-54 | | | | |
| Example 105 | | Compound | 3.72 | 21.39 | 219 | Red |
| | | 2-60 | | | | |
| Example 106 | Compound | Compound | 3.73 | 21.44 | 217 | Red |
| | 1-32 | 2-3 | | | | |
| Example 107 | | Compound | 3.70 | 21.64 | 206 | Red |
| | | 2-10 | | | | |
| Example 108 | | Compound | 3.64 | 21.52 | 214 | Red |
| | | 2-68 | | | | |
| Example 109 | | Compound | 3.68 | 21.52 | 215 | Red |
| | | 2-44 | | | | |
| Example 110 | | Compound | 3.64 | 21.44 | 234 | Red |
| | | 2-49 | | | | |
| Example 111 | Compound | Compound | 3.57 | 21.53 | 219 | Red |
| | 1-33 | 2-5 | | | | |
| Example 112 | | Compound | 3.64 | 21.38 | 218 | Red |
| | | 2-14 | | | | |
| Example 113 | | Compound | 3.67 | 21.71 | 223 | Red |
| | | 2-23 | | | | |
| Example 114 | | Compound | 3.63 | 22.16 | 220 | Red |
| | | 2-58 | | | | |
| Example 115 | | Compound | 3.54 | 21.21 | 218 | Red |
| | | 2-64 | | | | |
| Example 116 | Compound | Compound | 3.36 | 22.28 | 290 | Red |
| | 1-34 | 2-17 | | | | |
| Example 117 | | Compound | 3.37 | 21.77 | 274 | Red |
| | | 2-20 | | | | |
| Example 118 | | Compound | 3.35 | 21.83 | 277 | Red |
| | | 2-28 | | | | |
| Example 119 | | Compound | 3.41 | 21.89 | 265 | Red |
| | | 2-35 | | | | |
| Example 120 | | Compound | 3.46 | 21.86 | 269 | Red |
| | | 2-70 | | | | |
| Example 121 | Compound | Compound | 3.40 | 22.27 | 292 | Red |
| | 1-35 | 2-1 | | | | |
| Example 122 | | Compound | 3.35 | 21.91 | 260 | Red |
| | | 2-27 | | | | |
| Example 123 | | Compound | 3.42 | 22.17 | 288 | Red |
| | | 2-39 | | | | |
| Example 124 | | Compound | 3.39 | 22.07 | 277 | Red |
| | | 2-54 | | | | |
| Example 125 | | Compound | 3.35 | 21.33 | 281 | Red |
| | | 2-60 | | | | |
| Example 126 | Compound | Compound | 3.54 | 22.56 | 256 | Red |
| | 1-36 | 2-3 | | | | |
| Example 127 | | Compound | 3.45 | 22.83 | 247 | Red |
| | | 2-10 | | | | |
| Example 128 | | Compound | 3.55 | 23.12 | 237 | Red |
| | | 2-68 | | | | |
| Example 129 | | Compound | 3.53 | 22.51 | 237 | Red |
| | | 2-44 | | | | |
| Example 130 | | Compound | 3.46 | 22.30 | 239 | Red |
| | | 2-49 | | | | |
| Example 131 | Compound | Compound | 3.49 | 23.08 | 255 | Red |
| | 1-38 | 2-5 | | | | |
| Example 132 | | Compound | 3.45 | 23.11 | 252 | Red |
| | | 2-14 | | | | |
| Example 133 | | Compound | 3.56 | 22.43 | 233 | Red |
| | | 2-23 | | | | |
| Example 134 | | Compound | 3.45 | 22.65 | 267 | Red |
| | | 2-58 | | | | |
| Example 135 | | Compound | 3.50 | 22.70 | 256 | Red |
| | | 2-64 | | | | |

TABLE 4

| | | | | Effi- | | |
|---|---|---|---|---|---|---|
| | First | Second | Voltage | ciency | Lifetime | |
| | Host | Host | (V) | (cd/A) | T95(hr) | Color |
| Example 136 | Compound | Compound | 3.42 | 22.29 | 284 | Red |
| | 1-39 | 2-17 | | | | |
| Example 137 | | Compound | 3.40 | 21.58 | 258 | Red |
| | | 2-20 | | | | |
| Example 138 | | Compound | 3.46 | 21.57 | 291 | Red |
| | | 2-28 | | | | |
| Example 139 | | Compound | 3.40 | 21.48 | 262 | Red |
| | | 2-35 | | | | |
| Example 140 | | Compound | 3.44 | 21.43 | 267 | Red |
| | | 2-57 | | | | |
| Example 141 | Compound | Compound | 3.45 | 22.23 | 281 | Red |
| | 1-40 | 2-1 | | | | |
| Example 142 | | Compound | 3.38 | 21.54 | 271 | Red |
| | | 2-27 | | | | |
| Example 143 | | Compound | 3.40 | 21.56 | 267 | Red |
| | | 2-39 | | | | |
| Example 144 | | Compound | 3.35 | 21.91 | 265 | Red |
| | | 2-54 | | | | |
| Example 145 | | Compound | 3.40 | 21.33 | 265 | Red |
| | | 2-60 | | | | |
| Example 146 | Compound | Compound | 3.45 | 23.16 | 274 | Red |
| | 1-42 | 2-3 | | | | |
| Example 147 | | Compound | 3.36 | 23.98 | 285 | Red |
| | | 2-10 | | | | |
| Example 148 | | Compound | 3.47 | 23.81 | 274 | Red |
| | | 2-68 | | | | |
| Example 149 | | Compound | 3.41 | 24.14 | 266 | Red |
| | | 2-44 | | | | |
| Example 150 | | Compound | 3.43 | 23.04 | 265 | Red |
| | | 2-49 | | | | |
| Example 151 | Compound | Compound | 3.37 | 23.52 | 275 | Red |
| | 1-43 | 2-5 | | | | |
| Example 152 | | Compound | 3.44 | 23.20 | 273 | Red |
| | | 2-14 | | | | |
| Example 153 | | Compound | 3.42 | 23.57 | 274 | Red |
| | | 2-23 | | | | |
| Example 154 | | Compound | 3.35 | 23.84 | 256 | Red |
| | | 2-58 | | | | |
| Example 155 | | Compound | 3.44 | 23.09 | 264 | Red |
| | | 2-64 | | | | |
| Example 156 | Compound | Compound | 3.72 | 21.0 | 275 | Red |
| | 1-44 | 2-1 | | | | |
| Example 157 | | Compound | 3.70 | 22.1 | 271 | Red |
| | | 2-27 | | | | |
| Example 158 | | Compound | 3.75 | 21.5 | 284 | Red |
| | | 2-39 | | | | |
| Example 159 | | Compound | 3.74 | 21.3 | 268 | Red |
| | | 2-54 | | | | |
| Example 160 | | Compound | 3.71 | 22.3 | 275 | Red |
| | | 2-60 | | | | |
| Example 161 | Compound | Compound | 3.45 | 21.65 | 280 | Red |
| | 1-47 | 2-3 | | | | |
| Example 162 | | Compound | 3.37 | 21.49 | 261 | Red |
| | | 2-10 | | | | |
| Example 163 | | Compound | 3.44 | 21.78 | 257 | Red |
| | | 2-68 | | | | |
| Example 164 | | Compound | 3.41 | 21.25 | 262 | Red |
| | | 2-44 | | | | |
| Example 165 | | Compound | 3.47 | 21.48 | 283 | Red |
| | | 2-49 | | | | |
| Example 166 | Compound | Compound | 3.38 | 21.77 | 290 | Red |
| | 1-48 | 2-5 | | | | |
| Example 167 | | Compound | 3.42 | 21.61 | 274 | Red |
| | | 2-14 | | | | |
| Example 168 | | Compound | 3.43 | 21.59 | 259 | Red |
| | | 2-23 | | | | |
| Example 169 | | Compound | 3.42 | 22.16 | 290 | Red |
| | | 2-58 | | | | |
| Example 170 | | Compound | 3.43 | 21.47 | 287 | Red |
| | | 2-64 | | | | |
| Example 171 | Compound | Compound | 3.37 | 23.86 | 273 | Red |
| | 1-49 | 2-17 | | | | |
| Example 172 | | Compound | 3.42 | 23.92 | 284 | Red |
| | | 2-20 | | | | |
| Example 173 | | Compound | 3.44 | 23.41 | 279 | Red |
| | | 2-28 | | | | |
| Example 174 | | Compound | 3.38 | 23.45 | 258 | Red |
| | | 2-35 | | | | |
| Example 175 | | Compound | 3.40 | 24.15 | 273 | Red |
| | | 2-57 | | | | |
| Example 176 | Compound | Compound | 3.37 | 23.63 | 255 | Red |
| | 1-50 | 2-1 | | | | |
| Example 177 | | Compound | 3.43 | 24.11 | 262 | Red |
| | | 2-27 | | | | |
| Example 178 | | Compound | 3.41 | 23.51 | 282 | Red |
| | | 2-39 | | | | |
| Example 179 | | Compound | 3.38 | 23.92 | 276 | Red |
| | | 2-54 | | | | |
| Example 180 | | Compound | 3.38 | 24.07 | 255 | Red |
| | | 2-60 | | | | |

TABLE 5

| | | | | Effi- | | |
|---|---|---|---|---|---|---|
| | First | Second | Voltage | ciency | Lifetime | |
| | Host | Host | (V) | (cd/A) | T95(hr) | Color |
| Example 181 | Compound | Compound | 3.37 | 23.84 | 287 | Red |
| | 1-51 | 2-3 | | | | |
| Example 182 | | Compound | 3.35 | 23.27 | 272 | Red |
| | | 2-10 | | | | |
| Example 183 | | Compound | 3.45 | 23.37 | 292 | Red |
| | | 2-68 | | | | |
| Example 184 | | Compound | 3.47 | 23.41 | 262 | Red |
| | | 2-44 | | | | |
| Example 185 | | Compound | 3.46 | 23.25 | 271 | Red |
| | | 2-49 | | | | |
| Example 186 | Compound | Compound | 3.65 | 21.36 | 220 | Red |
| | 1-52 | 2-5 | | | | |
| Example 187 | | Compound | 3.61 | 21.58 | 206 | Red |
| | | 2-14 | | | | |
| Example 188 | | Compound | 3.61 | 21.78 | 224 | Red |
| | | 2-23 | | | | |
| Example 189 | | Compound | 3.60 | 22.24 | 208 | Red |
| | | 2-58 | | | | |
| Example 190 | | Compound | 3.66 | 21.30 | 223 | Red |
| | | 2-64 | | | | |
| Example 191 | Compound | Compound | 3.63 | 21.90 | 209 | Red |
| | 1-54 | 2-17 | | | | |
| Example 192 | | Compound | 3.65 | 21.38 | 220 | Red |
| | | 2-20 | | | | |
| Example 193 | | Compound | 3.59 | 21.76 | 205 | Red |
| | | 2-28 | | | | |
| Example 194 | | Compound | 3.55 | 21.57 | 233 | Red |
| | | 2-35 | | | | |
| Example 195 | | Compound | 3.53 | 21.57 | 230 | Red |
| | | 2-70 | | | | |
| Example 196 | Compound | Compound | 3.57 | 22.19 | 245 | Red |
| | 1-55 | 2-1 | | | | |
| Example 197 | | Compound | 3.56 | 22.09 | 226 | Red |
| | | 2-27 | | | | |
| Example 198 | | Compound | 3.60 | 21.40 | 220 | Red |
| | | 2-39 | | | | |
| Example 199 | | Compound | 3.58 | 21.28 | 225 | Red |
| | | 2-54 | | | | |
| Example 200 | | Compound | 3.65 | 21.53 | 225 | Red |
| | | 2-60 | | | | |
| Example 201 | Compound | Compound | 3.66 | 21.30 | 228 | Red |
| | 1-54 | 2-3 | | | | |
| Example 202 | | Compound | 3.60 | 21.53 | 220 | Red |
| | | 2-10 | | | | |
| Example 203 | | Compound | 3.56 | 22.17 | 242 | Red |
| | | 2-68 | | | | |
| Example 204 | | Compound | 3.52 | 22.12 | 241 | Red |
| | | 2-44 | | | | |
| Example 205 | | Compound | 3.58 | 21.87 | 227 | Red |
| | | 2-49 | | | | |
| Example 206 | Compound | Compound | 3.65 | 21.22 | 215 | Red |
| | 1-56 | 2-5 | | | | |
| Example 207 | | Compound | 3.58 | 22.20 | 233 | Red |
| | | 2-14 | | | | |
| Example 208 | | Compound | 3.53 | 21.24 | 210 | Red |
| | | 2-23 | | | | |
| Example 209 | | Compound | 3.64 | 21.30 | 210 | Red |
| | | 2-58 | | | | |
| Example 210 | | Compound | 3.52 | 21.57 | 225 | Red |
| | | 2-64 | | | | |
| Example 211 | Compound | Compound | 3.56 | 21.51 | 208 | Red |
| | 1-57 | 2-17 | | | | |
| Example 212 | | Compound | 3.54 | 21.57 | 226 | Red |
| | | 2-20 | | | | |
| Example 213 | | Compound | 3.63 | 21.57 | 205 | Red |
| | | 2-28 | | | | |
| Example 214 | | Compound | 3.61 | 22.30 | 214 | Red |
| | | 2-35 | | | | |
| Example 215 | | Compound | 3.61 | 21.71 | 217 | Red |
| | | 2-70 | | | | |
| Example 216 | Compound | Compound | 3.52 | 21.27 | 220 | Red |
| | 1-59 | 2-1 | | | | |
| Example 217 | | Compound | 3.56 | 21.89 | 243 | Red |
| | | 2-27 | | | | |
| Example 218 | | Compound | 3.58 | 22.03 | 216 | Red |
| | | 2-39 | | | | |
| Example 219 | | Compound | 3.64 | 22.02 | 219 | Red |
| | | 2-54 | | | | |
| Example 220 | | Compound | 3.55 | 22.00 | 235 | Red |
| | | 2-60 | | | | |

TABLE 6

| | | | | Effi- | | |
|---|---|---|---|---|---|---|
| | First | Second | Voltage | ciency | Lifetime | |
| | Host | Host | (V) | (cd/A) | T95(hr) | Color |
| Comparative | Compound | Compound | 4.10 | 17.64 | 123 | Red |
| Example 1 | B-1 | 2-1 | | | | |
| Comparative | | Compound | 4.12 | 18.54 | 105 | Red |
| Example 2 | | 2-27 | | | | |
| Comparative | | Compound | 4.12 | 18.57 | 113 | Red |
| Example 3 | | 2-39 | | | | |
| Comparative | | Compound | 4.02 | 18.23 | 126 | Red |
| Example 4 | | 2-54 | | | | |
| Comparative | | Compound | 4.01 | 17.39 | 112 | Red |
| Example 5 | | 2-60 | | | | |
| Comparative | Compound | Compound | 4.05 | 17.36 | 113 | Red |
| Example 6 | B-2 | 2-3 | | | | |
| Comparative | | Compound | 4.01 | 17.53 | 115 | Red |
| Example 7 | | 2-10 | | | | |
| Comparative | | Compound | 4.10 | 18.37 | 121 | Red |
| Example 8 | | 2-68 | | | | |
| Comparative | | Compound | 4.13 | 18.04 | 122 | Red |
| Example 9 | | 2-44 | | | | |
| Comparative | | Compound | 4.10 | 17.25 | 125 | Red |
| Example 10 | | 2-49 | | | | |
| Comparative | Compound | Compound | 4.09 | 17.74 | 122 | Red |
| Example 11 | B-3 | 2-5 | | | | |
| Comparative | | Compound | 4.10 | 18.00 | 118 | Red |
| Example 12 | | 2-14 | | | | |
| Comparative | | Compound | 4.10 | 18.50 | 106 | Red |
| Example 13 | | 2-23 | | | | |
| Comparative | | Compound | 4.07 | 18.30 | 113 | Red |
| Example 14 | | 2-58 | | | | |
| Comparative | | Compound | 4.05 | 17.39 | 121 | Red |
| Example 15 | | 2-64 | | | | |
| Comparative | Compound | Compound | 4.01 | 17.98 | 111 | Red |
| Example 16 | B-4 | 2-17 | | | | |
| Comparative | | Compound | 4.08 | 17.96 | 124 | Red |
| Example 17 | | 2-20 | | | | |
| Comparative | | Compound | 4.09 | 17.44 | 120 | Red |
| Example 18 | | 2-28 | | | | |
| Comparative | | Compound | 4.02 | 18.10 | 109 | Red |
| Example 19 | | 2-35 | | | | |
| Comparative | | Compound | 4.12 | 18.04 | 108 | Red |
| Example 20 | | 2-57 | | | | |
| Comparative | Compound | Compound | 4.10 | 16.81 | 95 | Red |
| Example 21 | B-5 | 2-1 | | | | |
| Comparative | | Compound | 4.12 | 17.15 | 103 | Red |
| Example 22 | | 2-27 | | | | |
| Comparative | | Compound | 4.14 | 16.81 | 93 | Red |
| Example 23 | | 2-39 | | | | |
| Comparative | | Compound | 4.11 | 16.99 | 93 | Red |
| Example 24 | | 2-54 | | | | |
| Comparative | | Compound | 4.16 | 17.13 | 103 | Red |
| Example 25 | | 2-60 | | | | |
| Comparative | Compound | Compound | 4.16 | 16.98 | 96 | Red |
| Example 26 | B-6 | 2-3 | | | | |
| Comparative | | Compound | 4.26 | 17.13 | 105 | Red |
| Example 27 | | 2-10 | | | | |
| Comparative | | Compound | 4.13 | 17.29 | 101 | Red |
| Example 28 | | 2-68 | | | | |
| Comparative | | Compound | 4.13 | 17.15 | 105 | Red |
| Example 29 | | 2-44 | | | | |
| Comparative | | Compound | 4.14 | 16.84 | 106 | Red |
| Example 30 | | 2-49 | | | | |

TABLE 7

| | | | | Effi- | | |
|---|---|---|---|---|---|---|
| | First | Second | Voltage | ciency | Lifetime | |
| | Host | Host | (V) | (cd/A) | T95(hr) | Color |
| Comparative | Compound | Compound | 4.11 | 18.12 | 142 | Red |
| Example 31 | B-7 | 2-5 | | | | |
| Comparative | | Compound | 4.05 | 17.77 | 139 | Red |
| Example 32 | | 2-14 | | | | |
| Comparative | | Compound | 4.08 | 17.90 | 138 | Red |
| Example 33 | | 2-23 | | | | |
| Comparative | | Compound | 4.09 | 17.30 | 152 | Red |
| Example 34 | | 2-58 | | | | |
| Comparative | | Compound | 4.03 | 17.99 | 136 | Red |
| Example 35 | | 2-64 | | | | |
| Comparative | Compound | Compound | 4.12 | 18.35 | 135 | Red |
| Example 36 | B-8 | 2-17 | | | | |
| Comparative | | Compound | 4.07 | 18.30 | 155 | Red |
| Example 37 | | 2-20 | | | | |
| Comparative | | Compound | 4.08 | 18.13 | 153 | Red |
| Example 38 | | 2-28 | | | | |
| Comparative | | Compound | 4.07 | 18.58 | 145 | Red |
| Example 39 | | 2-35 | | | | |
| Comparative | | Compound | 4.13 | 17.84 | 146 | Red |
| Example 40 | | 2-57 | | | | |
| Comparative | Compound | Compound | 4.09 | 18.14 | 151 | Red |
| Example 41 | B-9 | 2-1 | | | | |
| Comparative | | Compound | 4.08 | 18.00 | 146 | Red |
| Example 42 | | 2-27 | | | | |
| Comparative | | Compound | 4.05 | 17.48 | 141 | Red |
| Example 43 | | 2-39 | | | | |
| Comparative | | Compound | 4.08 | 18.56 | 142 | Red |
| Example 44 | | 2-54 | | | | |
| Comparative | | Compound | 4.01 | 17.49 | 158 | Red |
| Example 45 | | 2-60 | | | | |
| Comparative | Compound | Compound | 4.12 | 18.59 | 148 | Red |
| Example 46 | B-10 | 2-3 | | | | |
| Comparative | | Compound | 4.11 | 18.36 | 143 | Red |
| Example 47 | | 2-10 | | | | |
| Comparative | | Compound | 4.05 | 18.21 | 140 | Red |
| Example 48 | | 2-68 | | | | |
| Comparative | | Compound | 4.12 | 18.01 | 144 | Red |
| Example 49 | | 2-44 | | | | |
| Comparative | | Compound | 4.02 | 18.33 | 138 | Red |
| Example 50 | | 2-49 | | | | |
| Comparative | Compound | Compound | 4.04 | 18.25 | 122 | Red |
| Example 51 | B-11 | 2-5 | | | | |
| Comparative | | Compound | 4.09 | 17.49 | 115 | Red |
| Example 52 | | 2-14 | | | | |
| Comparative | | Compound | 4.08 | 17.88 | 112 | Red |
| Example 53 | | 2-23 | | | | |
| Comparative | | Compound | 4.10 | 17.55 | 117 | Red |
| Example 54 | | 2-58 | | | | |
| Comparative | | Compound | 4.04 | 18.53 | 116 | Red |
| Example 55 | | 2-64 | | | | |
| Comparative | Compound | Compound | 4.12 | 18.32 | 129 | Red |
| Example 56 | B-12 | 2-17 | | | | |
| Comparative | | Compound | 4.07 | 18.46 | 121 | Red |
| Example 57 | | 2-20 | | | | |
| Comparative | | Compound | 4.05 | 18.23 | 109 | Red |
| Example 58 | | 2-28 | | | | |
| Comparative | | Compound | 4.06 | 17.36 | 105 | Red |
| Example 59 | | 2-35 | | | | |
| Comparative | | Compound | 4.08 | 17.39 | 129 | Red |
| Example 60 | | 2-70 | | | | |

TABLE 8

| | | | | Effi- | | |
|---|---|---|---|---|---|---|
| | First | Second | Voltage | ciency | Lifetime | |
| | Host | Host | (V) | (cd/A) | T95(hr) | Color |
| Comparative | Compound 1- | Com- | 4.12 | 19.05 | 138 | Red |
| Example 61 | 2 | pound | | | | |
| Comparative | Compound 1- | C-1 | 4.02 | 18.86 | 141 | Red |
| Example 62 | 11 | | | | | |
| Comparative | Compound 1- | | 4.09 | 18.81 | 136 | Red |
| Example 63 | 15 | | | | | |
| Comparative | Compound 1- | | 4.10 | 18.53 | 142 | Red |
| Example 64 | 28 | | | | | |
| Comparative | Compound 1- | | 4.11 | 18.89 | 138 | Red |
| Example 65 | 33 | | | | | |
| Comparative | Compound 1- | | 4.01 | 18.87 | 135 | Red |
| Example 66 | 40 | | | | | |
| Comparative | Compound 1- | | 4.12 | 18.66 | 136 | Red |
| Example 67 | 43 | | | | | |
| Comparative | Compound 1- | | 4.10 | 19.14 | 139 | Red |
| Example 68 | 55 | | | | | |
| Comparative | Compound 1- | Com- | 4.11 | 18.40 | 152 | Red |
| Example 69 | 3 | pound | | | | |
| Comparative | Compound 1- | C-2 | 4.08 | 19.28 | 139 | Red |
| Example 70 | 7 | | | | | |
| Comparative | Compound 1- | | 4.07 | 15.3 | 109 | Red |
| Example 71 | 17 | | | | | |
| Comparative | Compound 1- | | 4.01 | 14.9 | 102 | Red |
| Example 72 | 24 | | | | | |
| Comparative | Compound 1- | | 4.06 | 15.7 | 123 | Red |
| Example 73 | 37 | | | | | |
| Comparative | Compound 1- | | 4.03 | 16.0 | 127 | Red |
| Example 74 | 47 | | | | | |
| Comparative | Compound 1- | | 4.11 | 15.4 | 120 | Red |
| Example 75 | 48 | | | | | |
| Comparative | Compound 1- | | 4.03 | 15.1 | 114 | Red |
| Example 76 | 58 | | | | | |
| Comparative | Compound 1- | Com- | 4.11 | 18.03 | 158 | Red |
| Example 77 | 9 | pound | | | | |
| Comparative | Compound 1- | C-3 | 4.05 | 17.66 | 150 | Red |
| Example 78 | 16 | | | | | |
| Comparative | Compound 1- | | 4.04 | 18.22 | 144 | Red |
| Example 79 | 22 | | | | | |
| Comparative | Compound 1- | | 4.13 | 17.78 | 134 | Red |
| Example 80 | 38 | | | | | |
| Comparative | Compound 1- | | 4.07 | 17.47 | 134 | Red |
| Example 81 | 41 | | | | | |
| Comparative | Compound 1- | | 4.04 | 17.27 | 148 | Red |
| Example 82 | 45 | | | | | |
| Comparative | Compound 1- | | 4.05 | 17.64 | 151 | Red |
| Example 83 | 53 | | | | | |
| Comparative | Compound 1- | | 4.04 | 18.51 | 135 | Red |
| Example 84 | 57 | | | | | |
| Comparative | Compound 1- | Com- | 4.12 | 18.33 | 137 | Red |
| Example 85 | 2 | pound | | | | |
| Comparative | Compound 1- | C-4 | 4.05 | 17.62 | 134 | Red |
| Example 86 | 14 | | | | | |
| Comparative | Compound 1- | | 4.03 | 17.31 | 144 | Red |
| Example 87 | 20 | | | | | |
| Comparative | Compound 1- | | 4.03 | 17.80 | 137 | Red |
| Example 88 | 27 | | | | | |
| Comparative | Compound 1- | | 4.07 | 17.59 | 152 | Red |
| Example 89 | 31 | | | | | |
| Comparative | Compound 1- | | 4.02 | 18.31 | 151 | Red |
| Example 90 | 44 | | | | | |
| Comparative | Compound 1- | | 4.10 | 17.87 | 146 | Red |
| Example 91 | 51 | | | | | |
| Comparative | Compound 1- | | 4.06 | 17.96 | 138 | Red |
| Example 92 | 59 | | | | | |
| Comparative | Compound 1- | Com- | 4.22 | 17.11 | 97 | Red |
| Example 93 | 2 | pound | | | | |
| Comparative | Compound 1- | C-5 | 4.21 | 17.38 | 100 | Red |
| Example 94 | 11 | | | | | |
| Comparative | Compound 1- | | 4.19 | 17.20 | 100 | Red |
| Example 95 | 15 | | | | | |
| Comparative | Compound 1- | | 4.09 | 16.85 | 102 | Red |
| Example 96 | 28 | | | | | |
| Comparative | Compound 1- | | 4.19 | 16.90 | 106 | Red |
| Example 97 | 33 | | | | | |
| Comparative | Compound 1- | | 4.18 | 17.29 | 101 | Red |
| Example 98 | 40 | | | | | |
| Comparative | Compound 1- | | 4.22 | 17.37 | 100 | Red |
| Example 99 | 43 | | | | | |
| Comparative | Compound 1- | | 4.21 | 17.18 | 98 | Red |
| Example 100 | 55 | | | | | |

TABLE 9

| | | | | Effi- | | |
|---|---|---|---|---|---|---|
| | First | Second | Voltage | ciency | Lifetime | |
| | Host | Host | (V) | (cd/A) | T95(hr) | Color |
| Comparative | Compound 1- | Com- | 4.11 | 17.36 | 104 | Red |
| Example 101 | 3 | pound | | | | |
| Comparative | Compound 1- | C-6 | 4.14 | 17.22 | 94 | Red |
| Example 102 | 7 | | | | | |
| Comparative | Compound 1- | | 4.18 | 17.31 | 95 | Red |
| Example 103 | 17 | | | | | |
| Comparative | Compound 1- | | 4.13 | 17.38 | 98 | Red |
| Example 104 | 24 | | | | | |
| Comparative | Compound 1- | | 4.17 | 17.33 | 102 | Red |
| Example 105 | 37 | | | | | |
| Comparative | Compound 1- | | 4.16 | 17.40 | 101 | Red |
| Example 106 | 47 | | | | | |
| Comparative | Compound 1- | | 4.17 | 16.99 | 99 | Red |
| Example 107 | 48 | | | | | |
| Comparative | Compound 1- | | 4.13 | 17.28 | 96 | Red |
| Example 108 | 58 | | | | | |
| Comparative | Compound 1- | Com- | 4.01 | 17.64 | 139 | Red |
| Example 109 | 9 | pound | | | | |
| Comparative | Compound 1- | C-7 | 4.09 | 18.24 | 155 | Red |
| Example 100 | 16 | | | | | |
| Comparative | Compound 1- | | 4.02 | 17.79 | 147 | Red |
| Example 111 | 22 | | | | | |
| Comparative | Compound 1- | | 4.08 | 17.20 | 138 | Red |
| Example 112 | 38 | | | | | |
| Comparative | Compound 1- | | 4.03 | 18.57 | 146 | Red |
| Example 113 | 41 | | | | | |
| Comparative | Compound 1- | | 4.04 | 17.67 | 154 | Red |
| Example 114 | 45 | | | | | |
| Comparative | Compound 1- | | 4.06 | 17.24 | 153 | Red |
| Example 115 | 53 | | | | | |
| Comparative | Compound 1- | | 4.07 | 17.51 | 135 | Red |
| Example 116 | 57 | | | | | |
| Comparative | Compound 1- | Com- | 4.06 | 17.74 | 111 | Red |
| Example 117 | 2 | pound | | | | |
| Comparative | Compound 1- | C-8 | 4.02 | 17.42 | 125 | Red |
| Example 118 | 14 | | | | | |
| Comparative | Compound 1- | | 4.07 | 17.90 | 127 | Red |
| Example 119 | 20 | | | | | |
| Comparative | Compound 1- | | 4.11 | 18.18 | 112 | Red |
| Example 120 | 27 | | | | | |
| Comparative | Compound 1- | | 4.09 | 17.63 | 103 | Red |
| Example 121 | 31 | | | | | |
| Comparative | Compound 1- | | 4.12 | 18.57 | 120 | Red |
| Example 122 | 44 | | | | | |
| Comparative | Compound 1- | | 4.12 | 18.27 | 107 | Red |
| Example 123 | 51 | | | | | |
| Comparative | Compound 1- | | 4.04 | 17.41 | 124 | Red |
| Example 124 | 59 | | | | | |
| Comparative | Compound 1- | Com- | 4.05 | 19.19 | 148 | Red |
| Example 125 | 2 | pound | | | | |
| Comparative | Compound 1- | C-9 | 4.11 | 19.06 | 159 | Red |
| Example 126 | 11 | | | | | |
| Comparative | Compound 1- | | 4.13 | 18.57 | 155 | Red |
| Example 127 | 15 | | | | | |
| Comparative | Compound 1- | | 4.05 | 18.65 | 158 | Red |
| Example 128 | 28 | | | | | |
| Comparative | Compound 1- | | 4.07 | 18.79 | 160 | Red |
| Example 129 | 33 | | | | | |
| Comparative | Compound 1- | | 4.01 | 19.23 | 150 | Red |
| Example 130 | 40 | | | | | |
| Comparative | Compound 1- | | 4.09 | 18.94 | 151 | Red |
| Example 131 | 43 | | | | | |
| Comparative | Compound 1- | | 4.01 | 19.13 | 146 | Red |
| Example 132 | 55 | | | | | |
| Comparative | Compound 1- | Com- | 4.03 | 18.22 | 108 | Red |
| Example 133 | 3 | pound | | | | |
| Comparative | Compound 1- | C-10 | 4.10 | 18.33 | 109 | Red |
| Example 134 | 7 | | | | | |
| Comparative | Compound 1- | | 4.03 | 18.08 | 113 | Red |
| Example 135 | 17 | | | | | |
| Comparative | Compound 1- | | 4.13 | 18.37 | 119 | Red |
| Example 136 | 24 | | | | | |
| Comparative | Compound 1- | | 4.06 | 18.38 | 114 | Red |
| Example 137 | 37 | | | | | |
| Comparative | Compound 1- | | 4.05 | 18.09 | 113 | Red |
| Example 138 | 47 | | | | | |
| Comparative | Compound 1- | | 4.02 | 18.05 | 113 | Red |
| Example 139 | 48 | | | | | |
| Comparative | Compound 1- | | 4.02 | 17.49 | 118 | Red |
| Example 140 | 58 | | | | | |

TABLE 10

| | | | | Effi- | | |
|---|---|---|---|---|---|---|
| | First | Second | Voltage | ciency | Lifetime | |
| | Host | Host | (V) | (cd/A) | T95(hr) | Color |
| Comparative | Compound 1- | Com- | 4.09 | 18.04 | 109 | Red |
| Example 141 | 9 | pound | | | | |
| Comparative | Compound 1- | C-11 | 4.09 | 17.78 | 103 | Red |
| Example 142 | 16 | | | | | |
| Comparative | Compound 1- | | 4.09 | 18.27 | 106 | Red |
| Example 143 | 22 | | | | | |
| Comparative | Compound 1- | | 4.01 | 18.43 | 107 | Red |
| Example 144 | 38 | | | | | |
| Comparative | Compound 1- | | 4.10 | 17.89 | 120 | Red |
| Example 145 | 41 | | | | | |
| Comparative | Compound 1- | | 4.06 | 17.82 | 116 | Red |
| Example 146 | 45 | | | | | |
| Comparative | Compound 1- | | 4.09 | 18.53 | 131 | Red |
| Example 147 | 53 | | | | | |
| Comparative | Compound 1- | | 4.07 | 17.33 | 119 | Red |
| Example 148 | 57 | | | | | |
| Comparative | Compound 1- | Com- | 4.03 | 17.21 | 150 | Red |
| Example 149 | 2 | pound | | | | |
| Comparative | Compound 1- | C-12 | 4.02 | 17.52 | 153 | Red |
| Example 150 | 14 | | | | | |
| Comparative | Compound 1- | | 4.07 | 18.30 | 154 | Red |
| Example 151 | 20 | | | | | |
| Comparative | Compound 1- | | 4.10 | 17.41 | 135 | Red |
| Example 152 | 27 | | | | | |
| Comparative | Compound 1- | | 4.10 | 17.54 | 152 | Red |
| Example 153 | 31 | | | | | |
| Comparative | Compound 1- | | 4.01 | 17.52 | 160 | Red |
| Example 154 | 44 | | | | | |
| Comparative | Compound 1- | | 4.05 | 17.77 | 151 | Red |
| Example 155 | 51 | | | | | |
| Comparative | Compound 1- | | 4.04 | 18.59 | 155 | Red |
| Example 156 | 59 | | | | | |

When a current was applied to the organic light emitting devices manufactured in Examples 1 to 220 and Comparative Examples 1 to 156, the results shown in Tables 1 to 10 were obtained. In the red organic light emitting device of Comparative Example 1, conventionally widely used materials were used, and Compound EB-1 was used as the electron blocking layer, and Dp-7 was used as the dopant for the red light emitting layer. When Comparative Examples Compounds B-1 to B-12 and the compound of Chemical Formula 2 of the present disclosure were co-deposited and used as a red light emitting layer as shown in Table 6 and Table 7, the driving voltage generally increased and the efficiency and lifetime decreased as compared with the combination of the present disclosure. Even when Comparative Example Compounds C-1 to C-12 and the compounds of Chemical Formula 1 of the present disclosure were co-deposited and used as a red light emitting layer as shown in Tables 8 to 10 above, the driving voltage increased, and the efficiency and lifetime decreased.

From the above results, it could be confirmed that when a combination of the compound of Chemical Formula 1, which is a first host, and the compound of Chemical Formula 2, which is a second host of the present disclosure, is used in an organic light emitting device, it is advantageous for energy transfer to the red dopant in the red light emitting layer, thus improving the driving voltage and increasing the efficiency and lifetime. Ultimately, this is considered to be because as compared with the combination with Comparative Example Compound, the combination of the compound of Chemical Formula 1 and the compound of Chemical Formula 2 of the present disclosure allows electrons and holes to combine to form excitons through a more stable balance within the light emitting layer, thus increasing efficiency and lifetime. In conclusion, it was confirmed that when the compound of Chemical Formula 1 and the compound of Chemical Formula 2 were combined and co-evaporated and used as a host for the red light emitting layer, the driving voltage, luminous efficiency and lifetime characteristics of the organic light emitting devices can be improved

Description of Symbols

1: substrate
2: anode
3: light emitting layer
4: cathode
5: hole injection layer
6: hole transport layer
7: electron blocking layer
8: hole blocking layer
9: electron injection and transport layer

1. An organic light emitting device comprising:
an anode;
a cathode; and
a light emitting layer interposed between the anode and the cathode,
wherein the light emitting layer includes a compound of the following Chemical Formula 1 and a compound of the following Chemical Formula 2: 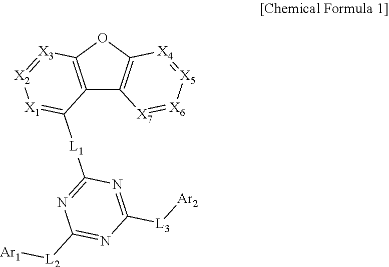
wherein in Chemical Formula 1:
X₁ to X₇ are each independently CR₁ or N, provided that at least one of X₁ to X₇ is N;
each R₁ is independently hydrogen, deuterium, a substituted or unsubstituted C₆₋₆₀ aryl, or a substituted or unsubstituted C₂₋₆₀ heteroaryl containing any one or more selected from the group consisting of N, O and S;
L₁ to L₃ are each independently a single bond, a substituted or unsubstituted C₆₋₆₀ arylene, or a substituted or unsubstituted C₂₋₆₀ heteroarylene containing at least one selected from the group consisting of N, O and S; and
Ar₁ and Ar₂ are each independently a substituted or unsubstituted C₆₋₆₀ aryl or a substituted or unsubstituted C₂₋₆₀ heteroaryl containing at least one heteroatom selected from the group consisting of N, O and S; 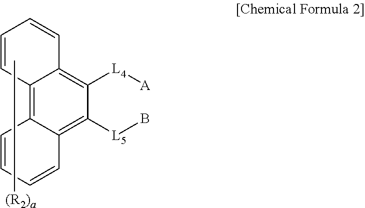
wherein in Chemical Formula 2:
L₄ and L₅ are each independently a single bond, a substituted or unsubstituted C₆₋₆₀ arylene, or a substituted or unsubstituted C₂₋₆₀ heteroarylene containing at least one heteroatom selected from the group consisting of N, O and S;
A and B are each independently hydrogen, deuterium, 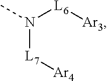
a substituted or unsubstituted C₆₋₆₀ aryl, or a substituted or unsubstituted C₂₋₆₀ heteroaryl containing at least one heteroatom selected from the group consisting of N, O and S, provided that at least one of A and B is 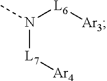
L₆ and L₇ are each independently a single bond, a substituted or unsubstituted C₆₋₆₀ arylene, or a substituted or unsubstituted C₂₋₆₀ heteroarylene containing at least one heteroatom selected from the group consisting of N, O and S;
Ar₃ and Ar₄ are each independently a substituted or unsubstituted C₆₋₆₀ aryl or a substituted or unsubstituted C₂₋₆₀ heteroaryl containing at least one heteroatom selected from the group consisting of N, O and S;
each R₂ is independently hydrogen or deuterium; and
a is an integer of 0 to 8.

2. The organic light emitting device according to claim 1, wherein:
any one of X₁ to X₇ is N, and the rest are CR₁.

3. The organic light emitting device according to claim 1, wherein:
R₁ is hydrogen, deuterium, phenyl, biphenylyl, naphthyl, carbazolyl, fluoranthenyl, phenanthrenyl, triphenylenyl, benzo[a]carbazolyl, benzo[b]carbazolyl, benzo[c]carbazolyl, dibenzofuranyl, benzo[d]naphtho[1,2-b]furanyl, benzo[d]naphtho[2,3-b]furanyl, benzo[d]naphtho[2,1-b]furanyl, benzo[d]naphtho[1,2-b]thiophenyl, benzo[d]naphtho[2,3-b]thiophenyl, benzo[d]naphtho[2,1-b]thiophenyl, benzo[c]phenanthrenyl, chrysenyl, phenyl naphthyl, or naphthyl phenyl,
provided that when the R₁ is not hydrogen or deuterium, R₁ is unsubstituted or substituted with at least one deuterium.

4. The organic light emitting device according to claim 1, wherein:
L₁ to L₃ are each independently a single bond, phenylene, naphthalendiyl, 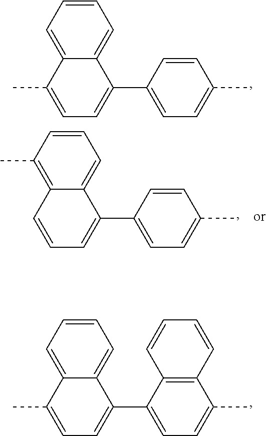
provided that when the L₁ to L₃ are not a single bond, L₁ to L₃ are unsubstituted or substituted with at least one deuterium.

5. The organic light emitting device according to claim 1, wherein:
Ar₁ and Ar₂ are each independently phenyl, biphenylyl, terphenylyl, naphthyl, fluoranthenyl, phenanthrenyl, dibenzofuranyl, dibenzothiophenyl, chrysenyl, or benzo[c]phenanthrenyl, and
the Ar₁ and Ar₂ are unsubstituted or substituted with at least one deuterium.

6. The organic light emitting device according to claim 1, wherein:
the compound of Chemical Formula 1 is any one compound selected from the group consisting of:

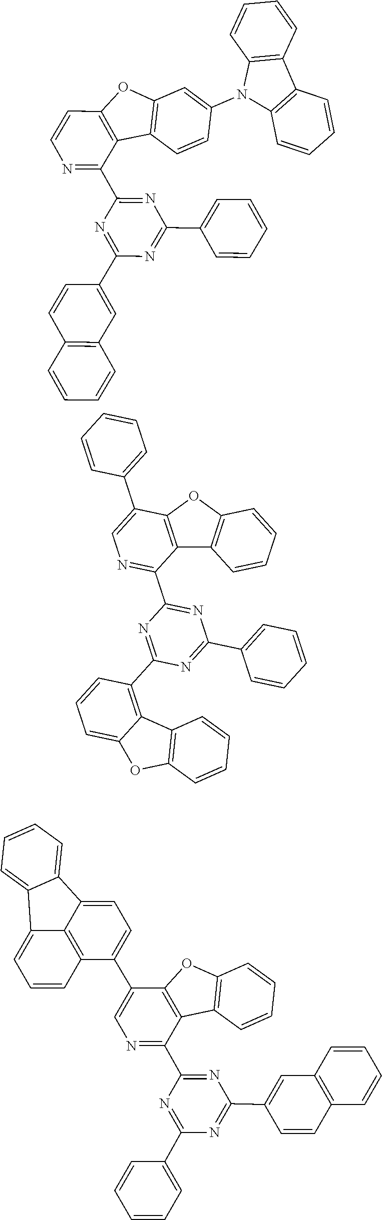

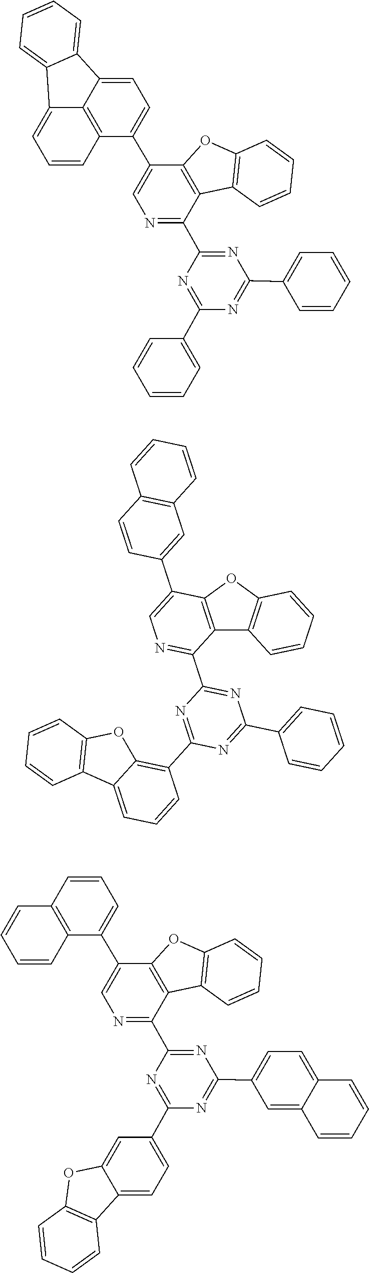

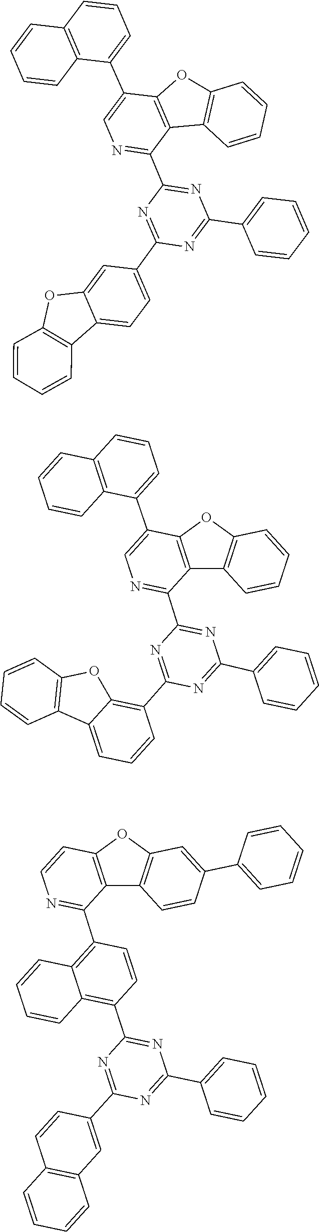 [SMILES: c1ccc(-c2nc(-c3ccc4c(c3)oc3ccccc34)nc(-c3ncc(-c4cccc5ccccc45)c4oc5ccccc5c34)n2)cc1 ; c1ccc(-c2nc(-c3cccc4c3oc3ccccc34)nc(-c3ncc(-c4cccc5ccccc45)c4oc5ccccc5c34)n2)cc1 ; c1ccc(-c2ccc3c(c2)oc2ccnc(-c4ccc(-c5nc(-c6ccccc6)nc(-c6ccc7ccccc7c6)n5)c5ccccc45)c23)cc1]

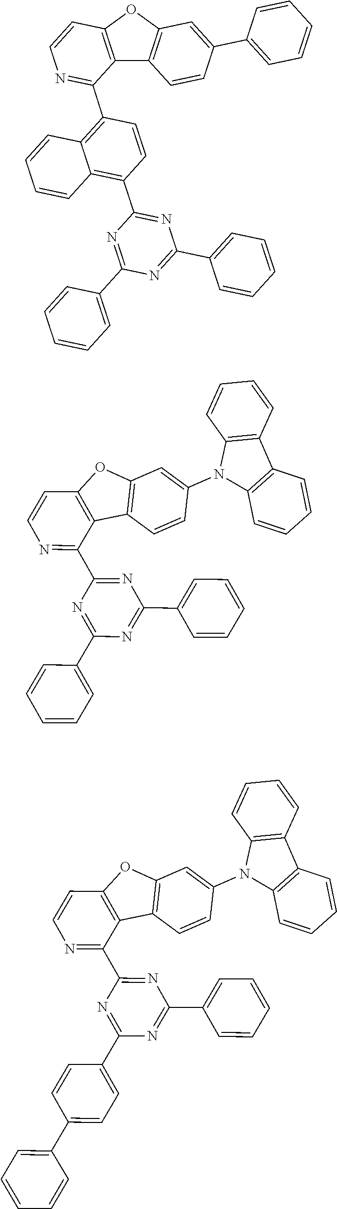 [SMILES: c1ccc(-c2ccc3c(c2)oc2ccnc(-c4ccc(-c5nc(-c6ccccc6)nc(-c6ccccc6)n5)c5ccccc45)c23)cc1 ; c1ccc(-c2nc(-c3ccccc3)nc(-c3nccc4oc5cc(-n6c7ccccc7c7ccccc76)ccc5c34)n2)cc1 ; c1ccc(-c2ccc(-c3nc(-c4ccccc4)nc(-c4nccc5oc6cc(-n7c8ccccc8c8ccccc87)ccc6c45)n3)cc2)cc1]

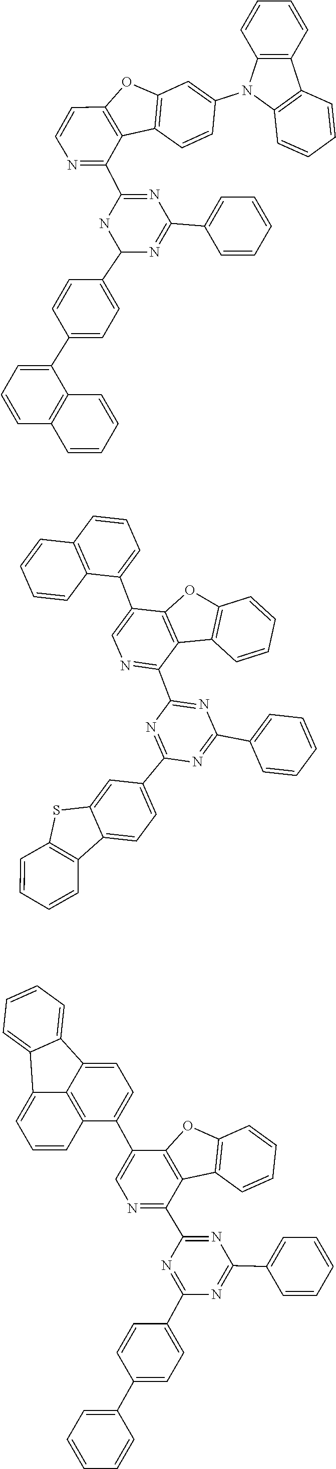

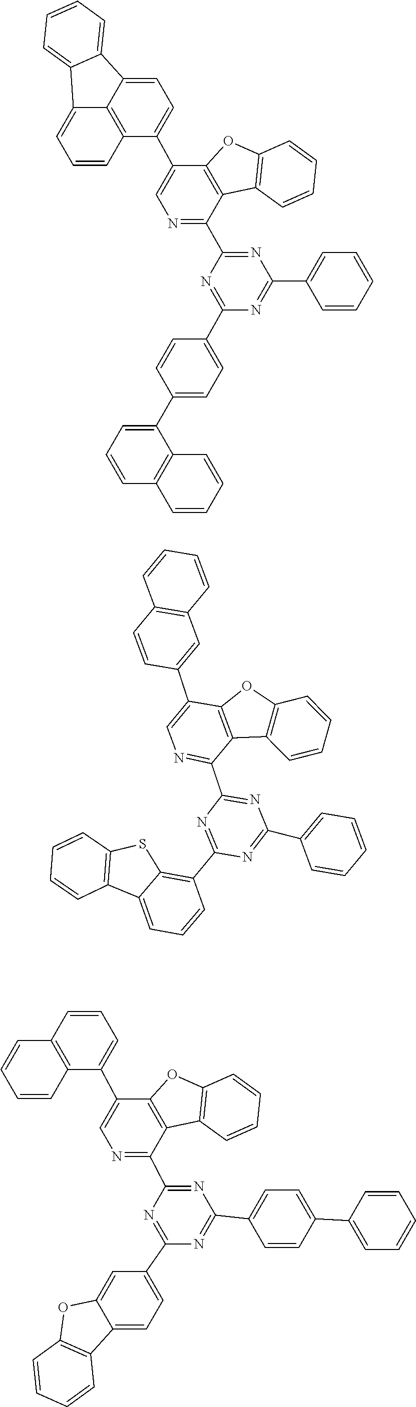

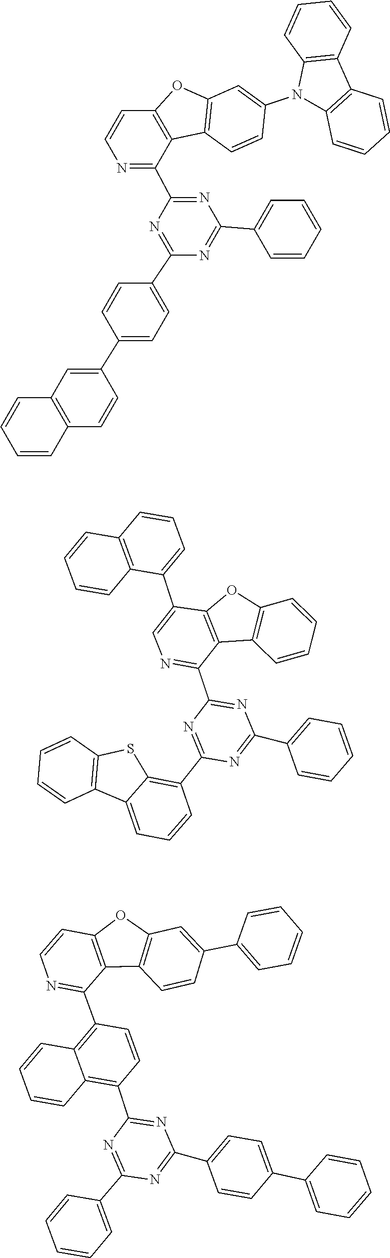 [SMILES: c1ccc(-c2nc(-c3ccc(-c4ccc5ccccc5c4)cc3)nc(-c3nccc4oc5cc(-n6c7ccccc7c7ccccc76)ccc5c34)n2)cc1 ; c1ccc(-c2nc(-c3cccc4c3sc3ccccc34)nc(-c3ncc(-c4cccc5ccccc45)c4oc5ccccc5c34)n2)cc1 ; c1ccc(-c2ccc(-c3nc(-c4ccccc4)nc(-c4ccc(-c5nccc6oc7cc(-c8ccccc8)ccc7c56)c5ccccc45)n3)cc2)cc1]

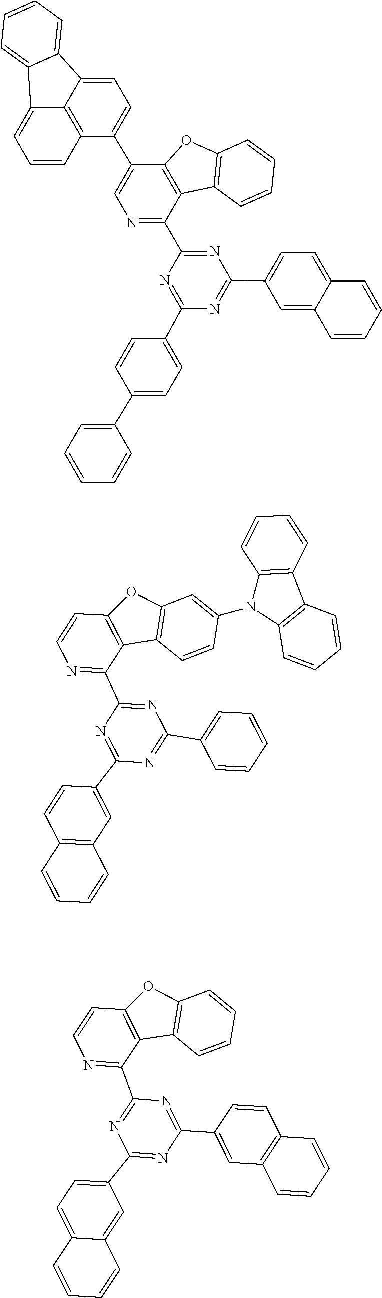 [SMILES: c1ccc(-c2ccc(-c3nc(-c4ccc5ccccc5c4)nc(-c4ncc(-c5ccc6c7c(cccc57)-c5ccccc5-6)c5oc6ccccc6c45)n3)cc2)cc1 ; c1ccc(-c2nc(-c3ccc4ccccc4c3)nc(-c3nccc4oc5cc(-n6c7ccccc7c7ccccc76)ccc5c34)n2)cc1 ; c1ccc2cc(-c3nc(-c4ccc5ccccc5c4)nc(-c4nccc5oc6ccccc6c45)n3)ccc2c1]

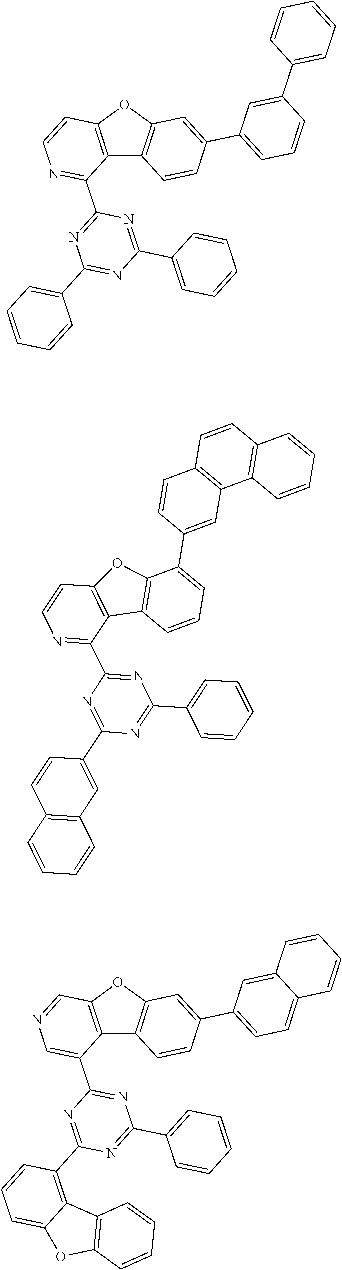 [SMILES: c1ccc(-c2cccc(-c3ccc4c(c3)oc3ccnc(-c5nc(-c6ccccc6)nc(-c6ccccc6)n5)c34)c2)cc1 ; c1ccc(-c2nc(-c3ccc4ccccc4c3)nc(-c3nccc4oc5c(-c6ccc7ccc8ccccc8c7c6)cccc5c34)n2)cc1 ; c1ccc(-c2nc(-c3cccc4oc5ccccc5c34)nc(-c3cncc4oc5cc(-c6ccc7ccccc7c6)ccc5c34)n2)cc1]

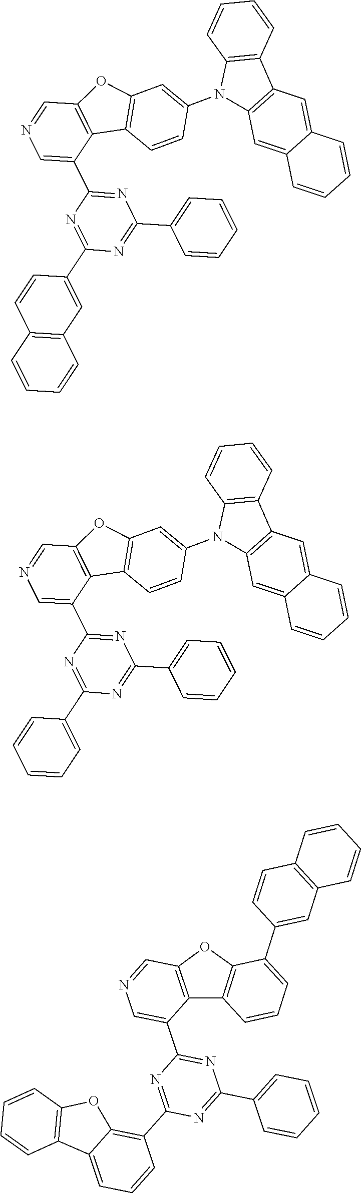 [SMILES: c1ccc(-c2nc(-c3ccc4ccccc4c3)nc(-c3cncc4oc5cc(-n6c7ccccc7c7cc8ccccc8cc76)ccc5c34)n2)cc1 ; c1ccc(-c2nc(-c3ccccc3)nc(-c3cncc4oc5cc(-n6c7ccccc7c7cc8ccccc8cc76)ccc5c34)n2)cc1 ; c1ccc(-c2nc(-c3cccc4c3oc3ccccc34)nc(-c3cncc4oc5c(-c6ccc7ccccc7c6)cccc5c34)n2)cc1]

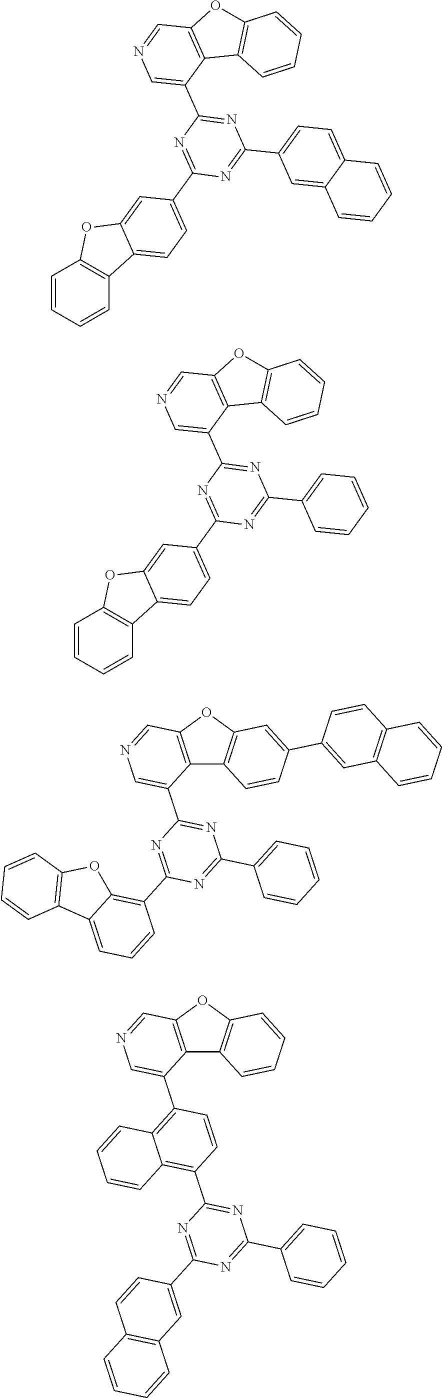 [SMILES: c1ccc2cc(-c3nc(-c4ccc5c(c4)oc4ccccc45)nc(-c4cncc5oc6ccccc6c45)n3)ccc2c1 ; c1ccc(-c2nc(-c3ccc4c(c3)oc3ccccc34)nc(-c3cncc4oc5ccccc5c34)n2)cc1 ; c1ccc(-c2nc(-c3cccc4c3oc3ccccc34)nc(-c3cncc4oc5cc(-c6ccc7ccccc7c6)ccc5c34)n2)cc1 ; c1ccc(-c2nc(-c3ccc4ccccc4c3)nc(-c3ccc(-c4cncc5oc6ccccc6c45)c4ccccc34)n2)cc1]

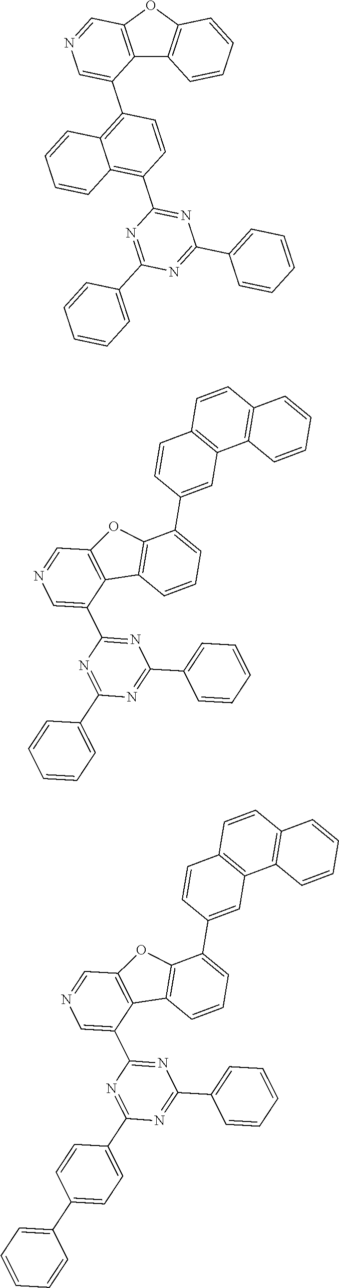 [SMILES: c1ccc(-c2nc(-c3ccccc3)nc(-c3ccc(-c4cncc5oc6ccccc6c45)c4ccccc34)n2)cc1 ; c1ccc(-c2nc(-c3ccccc3)nc(-c3cncc4oc5c(-c6ccc7ccc8ccccc8c7c6)cccc5c34)n2)cc1 ; c1ccc(-c2ccc(-c3nc(-c4ccccc4)nc(-c4cncc5oc6c(-c7ccc8ccc9ccccc9c8c7)cccc6c45)n3)cc2)cc1]

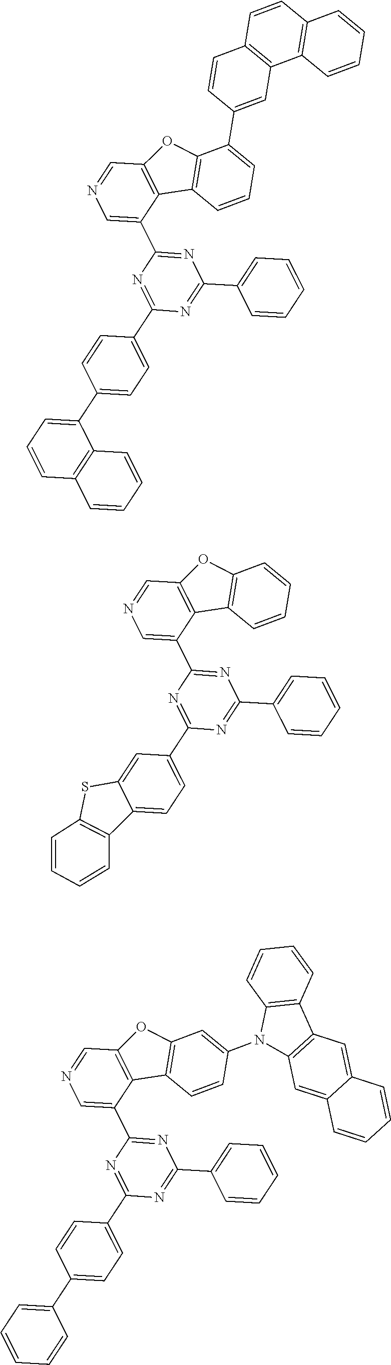 [SMILES: c1ccc(-c2nc(-c3ccc(-c4cccc5ccccc45)cc3)nc(-c3cncc4oc5c(-c6ccc7ccc8ccccc8c7c6)cccc5c34)n2)cc1 ; c1ccc(-c2nc(-c3ccc4c(c3)sc3ccccc34)nc(-c3cncc4oc5ccccc5c34)n2)cc1 ; c1ccc(-c2ccc(-c3nc(-c4ccccc4)nc(-c4cncc5oc6cc(-n7c8ccccc8c8cc9ccccc9cc87)ccc6c45)n3)cc2)cc1]

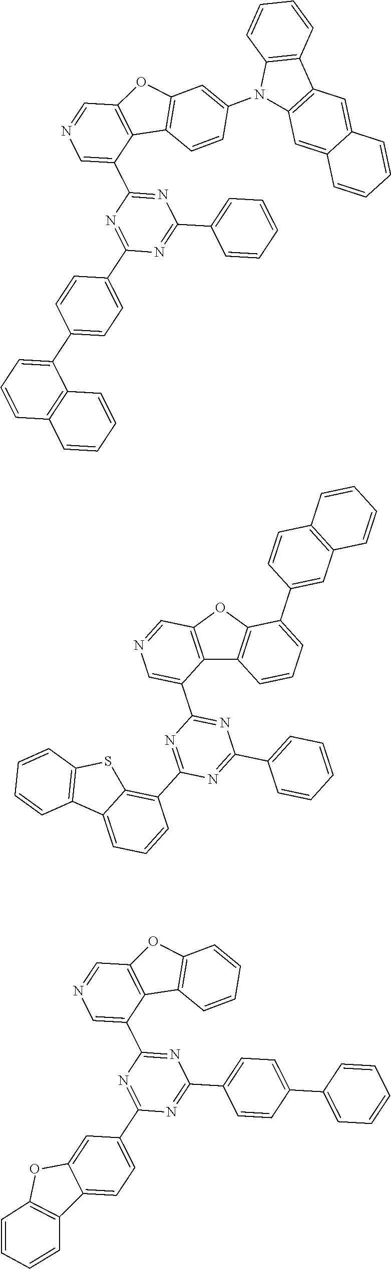 [SMILES: c1ccc(-c2nc(-c3ccc(-c4cccc5ccccc45)cc3)nc(-c3cncc4oc5cc(-n6c7ccccc7c7cc8ccccc8cc76)ccc5c34)n2)cc1 ; c1ccc(-c2nc(-c3cccc4c3sc3ccccc34)nc(-c3cncc4oc5c(-c6ccc7ccccc7c6)cccc5c34)n2)cc1 ; c1ccc(-c2ccc(-c3nc(-c4ccc5c(c4)oc4ccccc45)nc(-c4cncc5oc6ccccc6c45)n3)cc2)cc1]

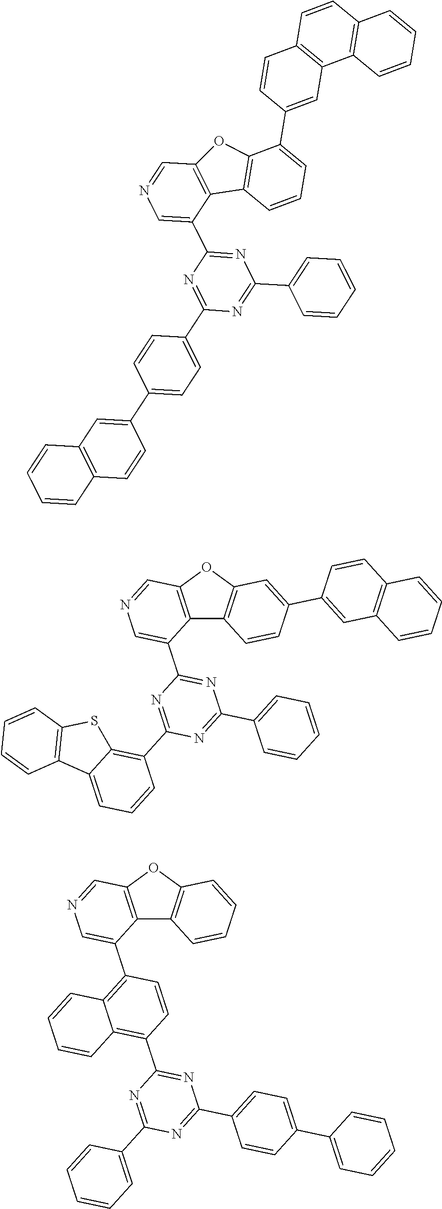 [SMILES: c1ccc(-c2nc(-c3ccc(-c4ccc5ccccc5c4)cc3)nc(-c3cncc4oc5c(-c6ccc7ccc8ccccc8c7c6)cccc5c34)n2)cc1 ; c1ccc(-c2nc(-c3cccc4c3sc3ccccc34)nc(-c3cncc4oc5cc(-c6ccc7ccccc7c6)ccc5c34)n2)cc1 ; c1ccc(-c2ccc(-c3nc(-c4ccccc4)nc(-c4ccc(-c5cncc6oc7ccccc7c56)c5ccccc45)n3)cc2)cc1]

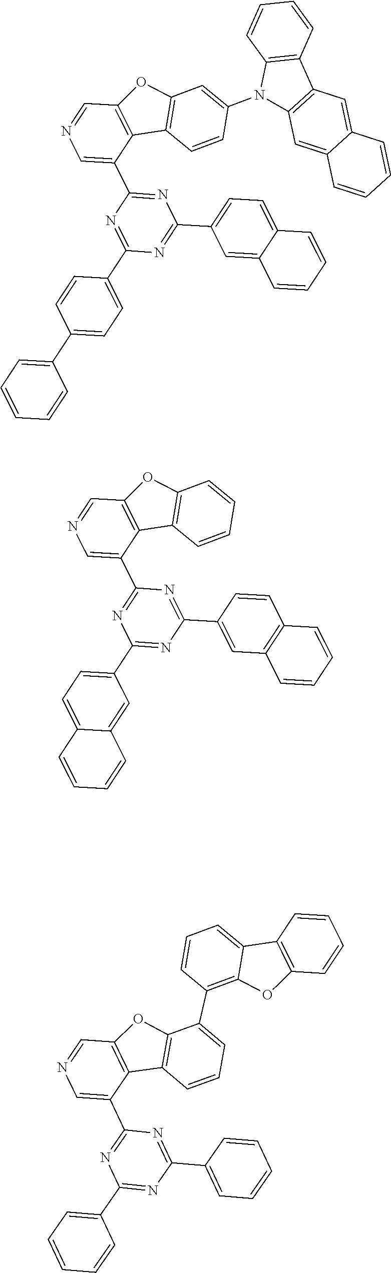 [SMILES: c1ccc(-c2ccc(-c3nc(-c4ccc5ccccc5c4)nc(-c4cncc5oc6cc(-n7c8ccccc8c8cc9ccccc9cc87)ccc6c45)n3)cc2)cc1 ; c1ccc2cc(-c3nc(-c4ccc5ccccc5c4)nc(-c4cncc5oc6ccccc6c45)n3)ccc2c1 ; c1ccc(-c2nc(-c3ccccc3)nc(-c3cncc4oc5c(-c6cccc7c6oc6ccccc67)cccc5c34)n2)cc1]

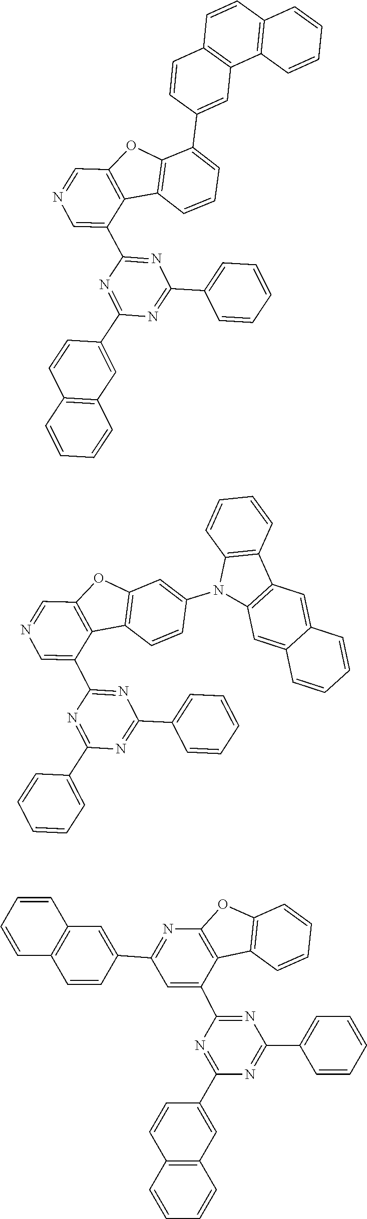 [SMILES: c1ccc(-c2nc(-c3ccc4ccccc4c3)nc(-c3cncc4oc5c(-c6ccc7ccc8ccccc8c7c6)cccc5c34)n2)cc1 ; c1ccc(-c2nc(-c3ccccc3)nc(-c3cncc4oc5cc(-n6c7ccccc7c7cc8ccccc8cc76)ccc5c34)n2)cc1 ; c1ccc(-c2nc(-c3ccc4ccccc4c3)nc(-c3cc(-c4ccc5ccccc5c4)nc4oc5ccccc5c34)n2)cc1]

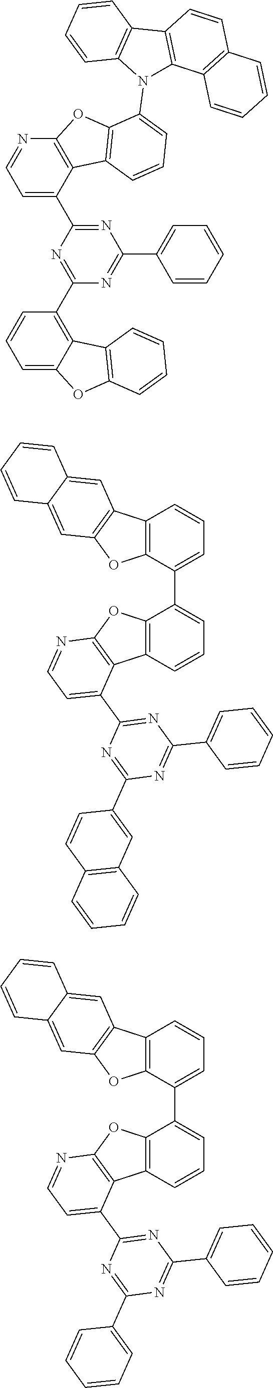 [SMILES: c1ccc(-c2nc(-c3cccc4oc5ccccc5c34)nc(-c3ccnc4oc5c(-n6c7ccccc7c7ccc8ccccc8c76)cccc5c34)n2)cc1 ; c1ccc(-c2nc(-c3ccc4ccccc4c3)nc(-c3ccnc4oc5c(-c6cccc7c6oc6cc8ccccc8cc67)cccc5c34)n2)cc1 ; c1ccc(-c2nc(-c3ccccc3)nc(-c3ccnc4oc5c(-c6cccc7c6oc6cc8ccccc8cc67)cccc5c34)n2)cc1]

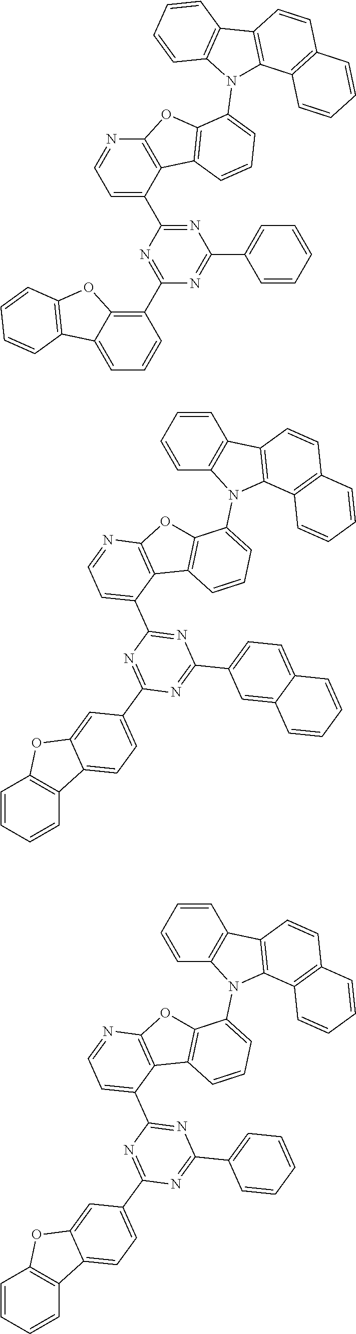 [SMILES: c1ccc(-c2nc(-c3cccc4c3oc3ccccc34)nc(-c3ccnc4oc5c(-n6c7ccccc7c7ccc8ccccc8c76)cccc5c34)n2)cc1 ; c1ccc2cc(-c3nc(-c4ccc5c(c4)oc4ccccc45)nc(-c4ccnc5oc6c(-n7c8ccccc8c8ccc9ccccc9c87)cccc6c45)n3)ccc2c1 ; c1ccc(-c2nc(-c3ccc4c(c3)oc3ccccc34)nc(-c3ccnc4oc5c(-n6c7ccccc7c7ccc8ccccc8c76)cccc5c34)n2)cc1]

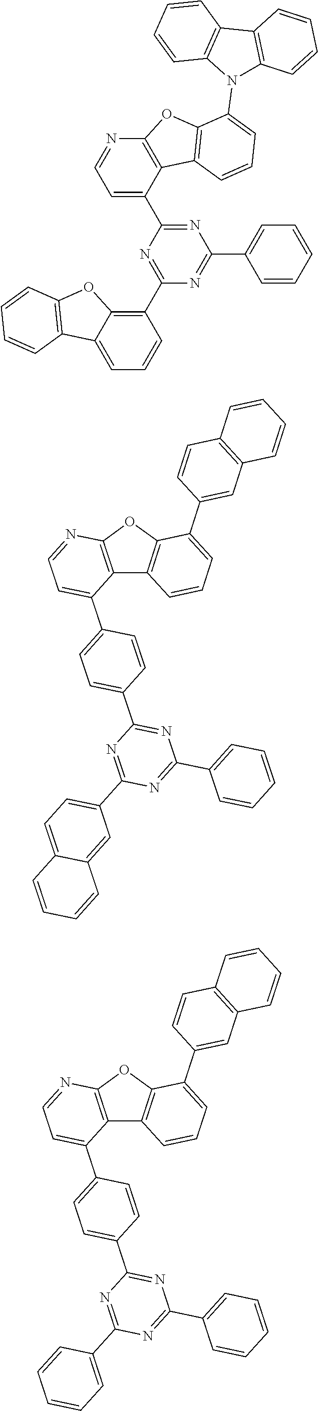 [SMILES: c1ccc(-c2nc(-c3cccc4c3oc3ccccc34)nc(-c3ccnc4oc5c(-n6c7ccccc7c7ccccc76)cccc5c34)n2)cc1 ; c1ccc(-c2nc(-c3ccc(-c4ccnc5oc6c(-c7ccc8ccccc8c7)cccc6c45)cc3)nc(-c3ccc4ccccc4c3)n2)cc1 ; c1ccc(-c2nc(-c3ccccc3)nc(-c3ccc(-c4ccnc5oc6c(-c7ccc8ccccc8c7)cccc6c45)cc3)n2)cc1]

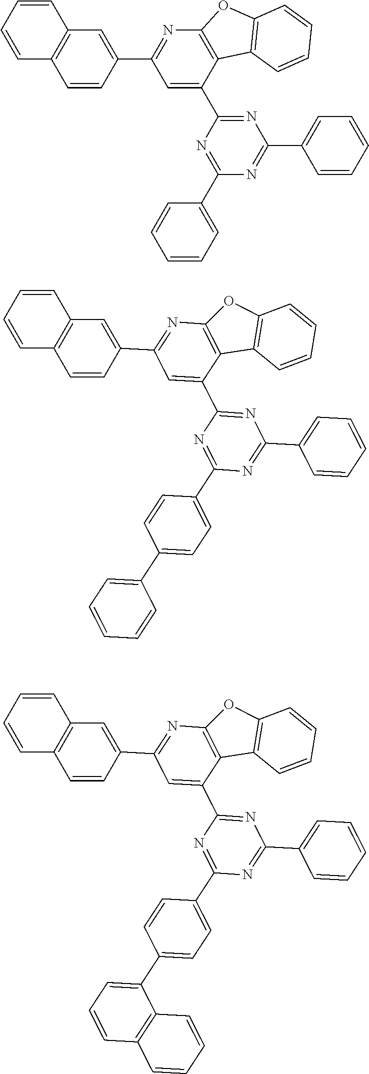 [SMILES: c1ccc(-c2nc(-c3ccccc3)nc(-c3cc(-c4ccc5ccccc5c4)nc4oc5ccccc5c34)n2)cc1 ; c1ccc(-c2ccc(-c3nc(-c4ccccc4)nc(-c4cc(-c5ccc6ccccc6c5)nc5oc6ccccc6c45)n3)cc2)cc1 ; c1ccc(-c2nc(-c3ccc(-c4cccc5ccccc45)cc3)nc(-c3cc(-c4ccc5ccccc5c4)nc4oc5ccccc5c34)n2)cc1]

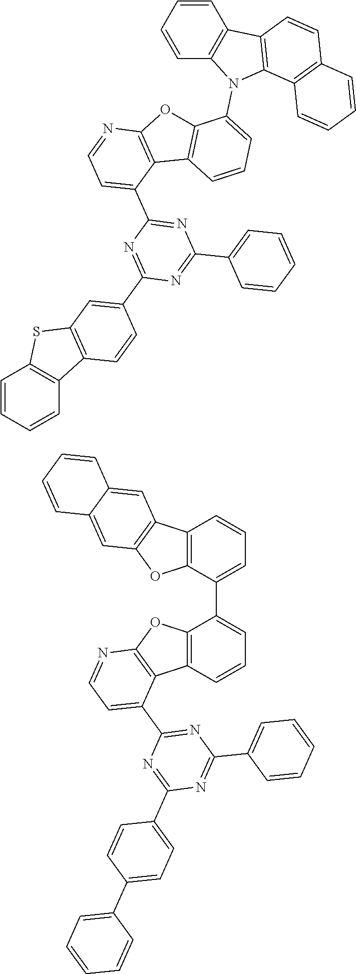

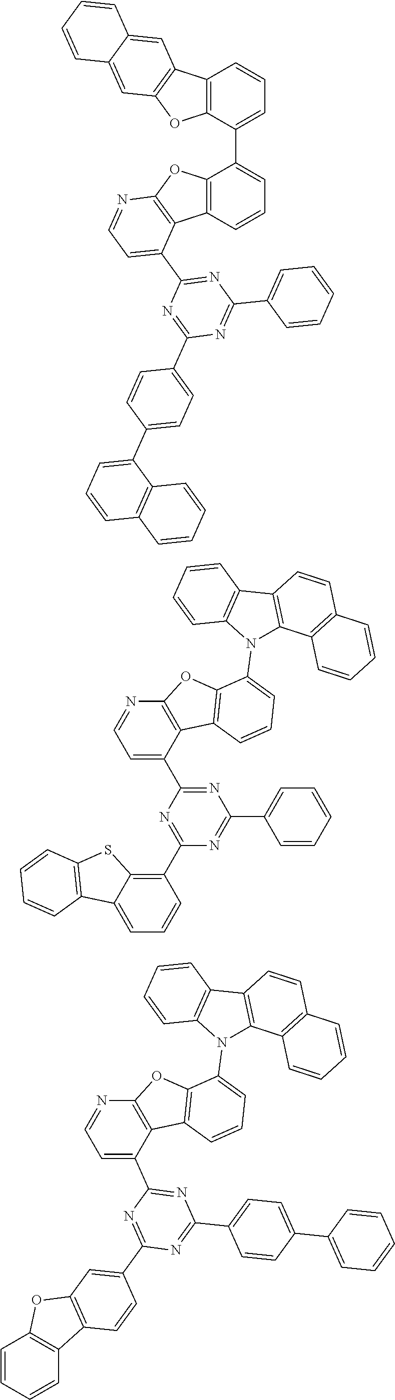

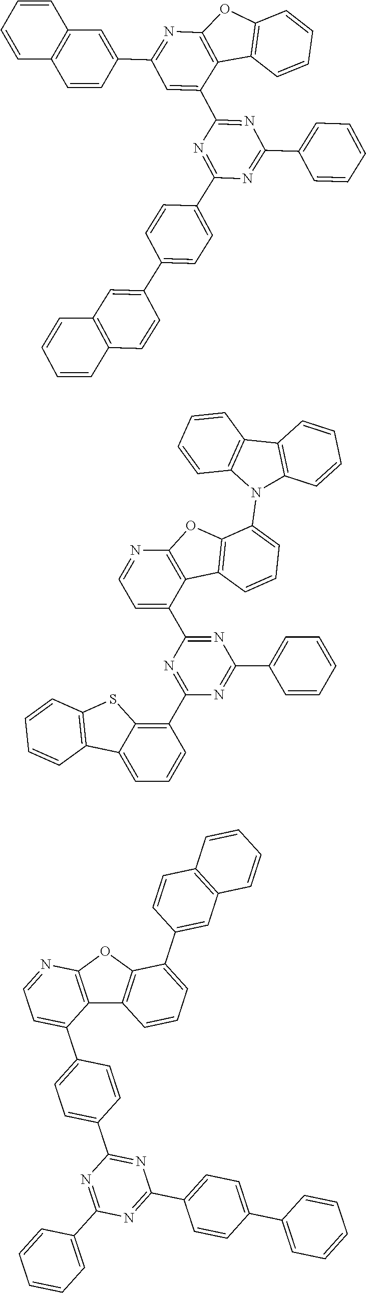 [SMILES: c1ccc(-c2nc(-c3ccc(-c4ccc5ccccc5c4)cc3)nc(-c3cc(-c4ccc5ccccc5c4)nc4oc5ccccc5c34)n2)cc1 ; c1ccc(-c2nc(-c3cccc4c3sc3ccccc34)nc(-c3ccnc4oc5c(-n6c7ccccc7c7ccccc76)cccc5c34)n2)cc1 ; c1ccc(-c2ccc(-c3nc(-c4ccccc4)nc(-c4ccc(-c5ccnc6oc7c(-c8ccc9ccccc9c8)cccc7c56)cc4)n3)cc2)cc1]

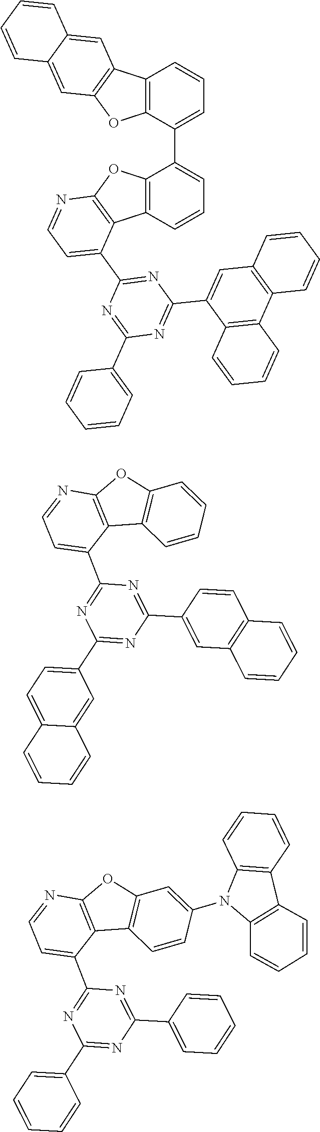 [SMILES: c1ccc(-c2nc(-c3cc4ccccc4c4ccccc34)nc(-c3ccnc4oc5c(-c6cccc7c6oc6cc8ccccc8cc67)cccc5c34)n2)cc1 ; c1ccc2cc(-c3nc(-c4ccc5ccccc5c4)nc(-c4ccnc5oc6ccccc6c45)n3)ccc2c1 ; c1ccc(-c2nc(-c3ccccc3)nc(-c3ccnc4oc5cc(-n6c7ccccc7c7ccccc76)ccc5c34)n2)cc1]

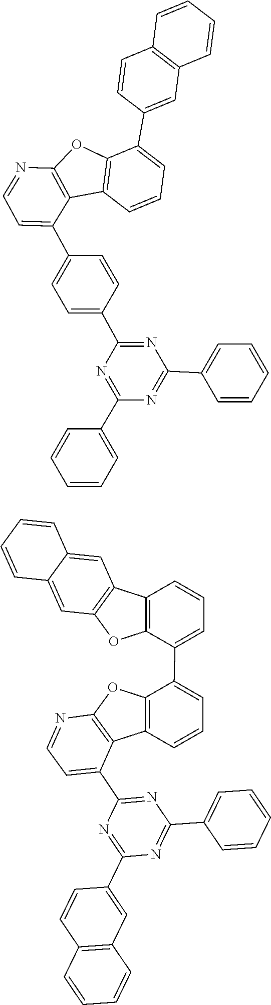

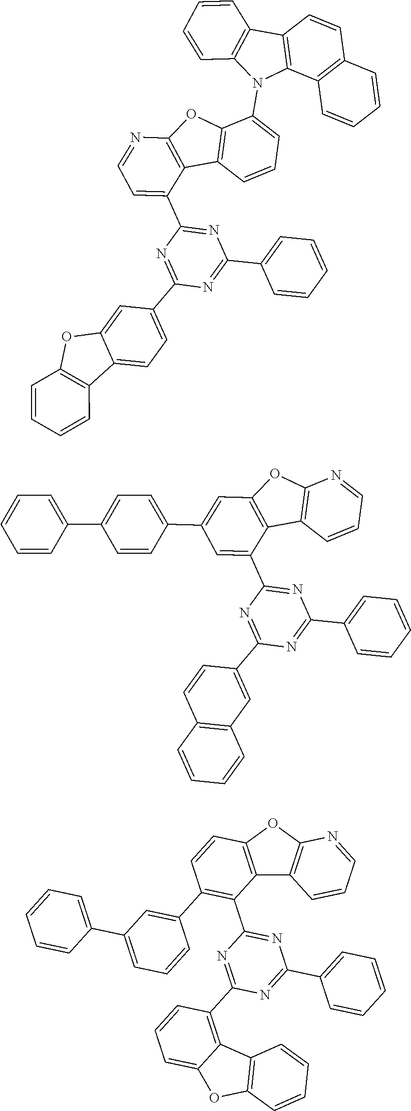 [SMILES: c1ccc(-c2nc(-c3ccc4c(c3)oc3ccccc34)nc(-c3ccnc4oc5c(-n6c7ccccc7c7ccc8ccccc8c76)cccc5c34)n2)cc1 ; c1ccc(-c2ccc(-c3cc(-c4nc(-c5ccccc5)nc(-c5ccc6ccccc6c5)n4)c4c(c3)oc3ncccc34)cc2)cc1 ; c1ccc(-c2cccc(-c3ccc4oc5ncccc5c4c3-c3nc(-c4ccccc4)nc(-c4cccc5oc6ccccc6c45)n3)c2)cc1]

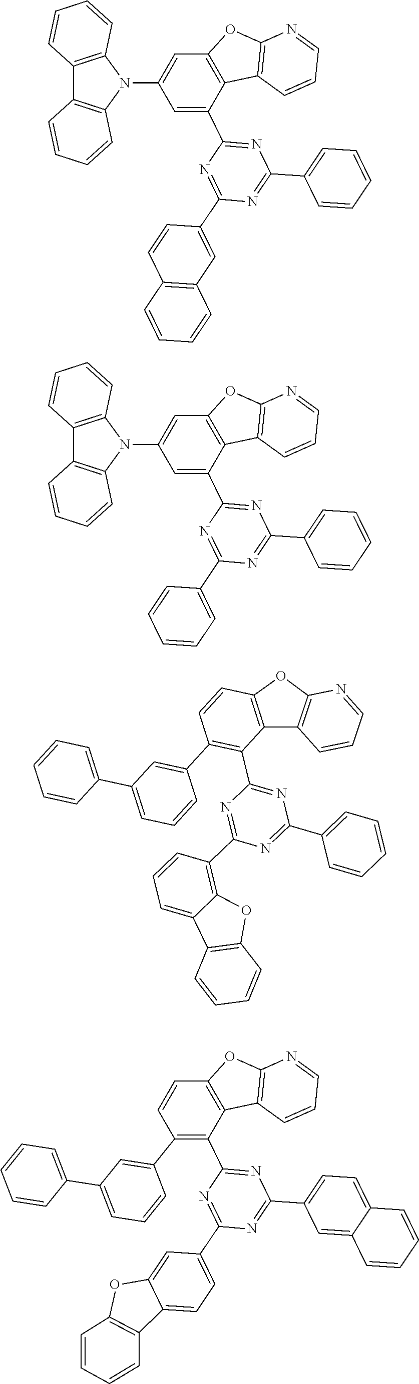 [SMILES: c1ccc(-c2nc(-c3ccc4ccccc4c3)nc(-c3cc(-n4c5ccccc5c5ccccc54)cc4oc5ncccc5c34)n2)cc1 ; c1ccc(-c2nc(-c3ccccc3)nc(-c3cc(-n4c5ccccc5c5ccccc54)cc4oc5ncccc5c34)n2)cc1 ; c1ccc(-c2cccc(-c3ccc4oc5ncccc5c4c3-c3nc(-c4ccccc4)nc(-c4cccc5c4oc4ccccc45)n3)c2)cc1 ; c1ccc(-c2cccc(-c3ccc4oc5ncccc5c4c3-c3nc(-c4ccc5ccccc5c4)nc(-c4ccc5c(c4)oc4ccccc45)n3)c2)cc1]

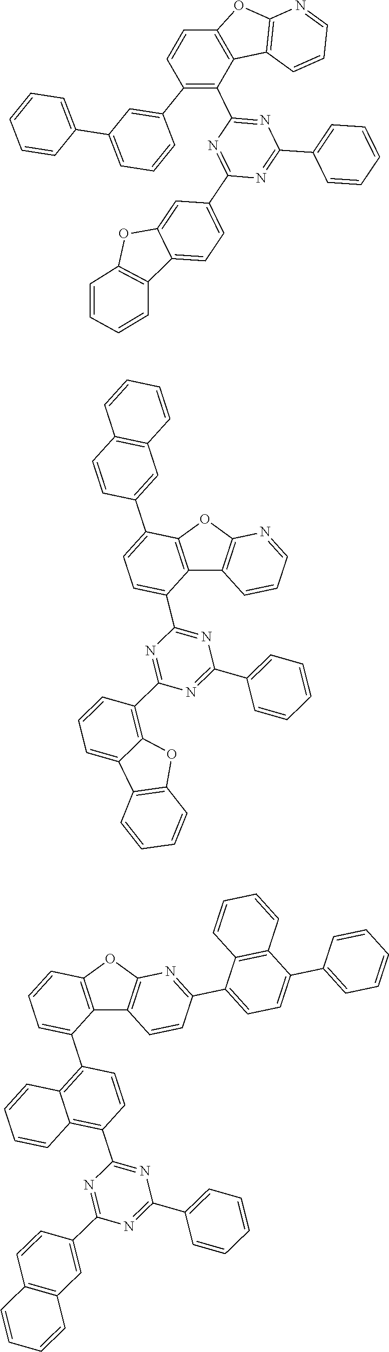 [SMILES: c1ccc(-c2cccc(-c3ccc4oc5ncccc5c4c3-c3nc(-c4ccccc4)nc(-c4ccc5c(c4)oc4ccccc45)n3)c2)cc1 ; c1ccc(-c2nc(-c3cccc4c3oc3ccccc34)nc(-c3ccc(-c4ccc5ccccc5c4)c4oc5ncccc5c34)n2)cc1 ; c1ccc(-c2nc(-c3ccc4ccccc4c3)nc(-c3ccc(-c4cccc5oc6nc(-c7ccc(-c8ccccc8)c8ccccc78)ccc6c45)c4ccccc34)n2)cc1]

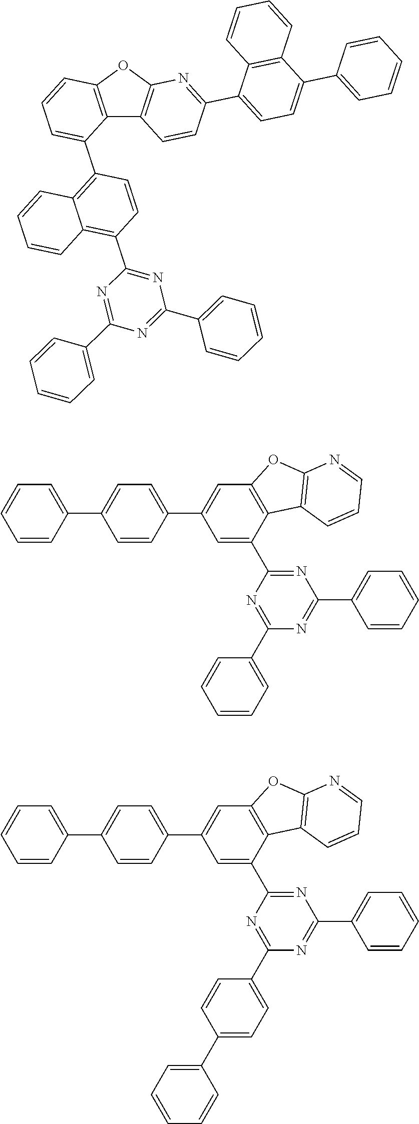 [SMILES: c1ccc(-c2nc(-c3ccccc3)nc(-c3ccc(-c4cccc5oc6nc(-c7ccc(-c8ccccc8)c8ccccc78)ccc6c45)c4ccccc34)n2)cc1 ; c1ccc(-c2ccc(-c3cc(-c4nc(-c5ccccc5)nc(-c5ccccc5)n4)c4c(c3)oc3ncccc34)cc2)cc1 ; c1ccc(-c2ccc(-c3cc(-c4nc(-c5ccccc5)nc(-c5ccc(-c6ccccc6)cc5)n4)c4c(c3)oc3ncccc34)cc2)cc1]

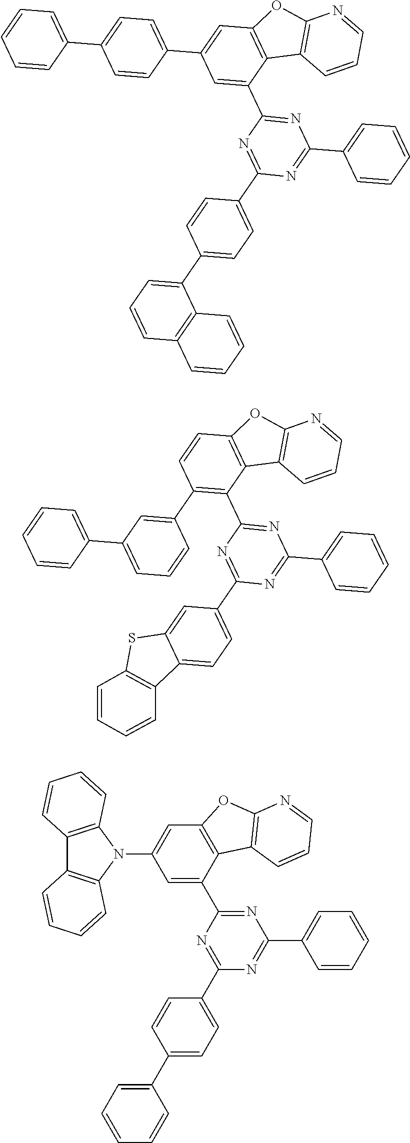 [SMILES: c1ccc(-c2ccc(-c3cc(-c4nc(-c5ccccc5)nc(-c5ccc(-c6cccc7ccccc67)cc5)n4)c4c(c3)oc3ncccc34)cc2)cc1 ; c1ccc(-c2cccc(-c3ccc4oc5ncccc5c4c3-c3nc(-c4ccccc4)nc(-c4ccc5c(c4)sc4ccccc45)n3)c2)cc1 ; c1ccc(-c2ccc(-c3nc(-c4ccccc4)nc(-c4cc(-n5c6ccccc6c6ccccc65)cc5oc6ncccc6c45)n3)cc2)cc1]

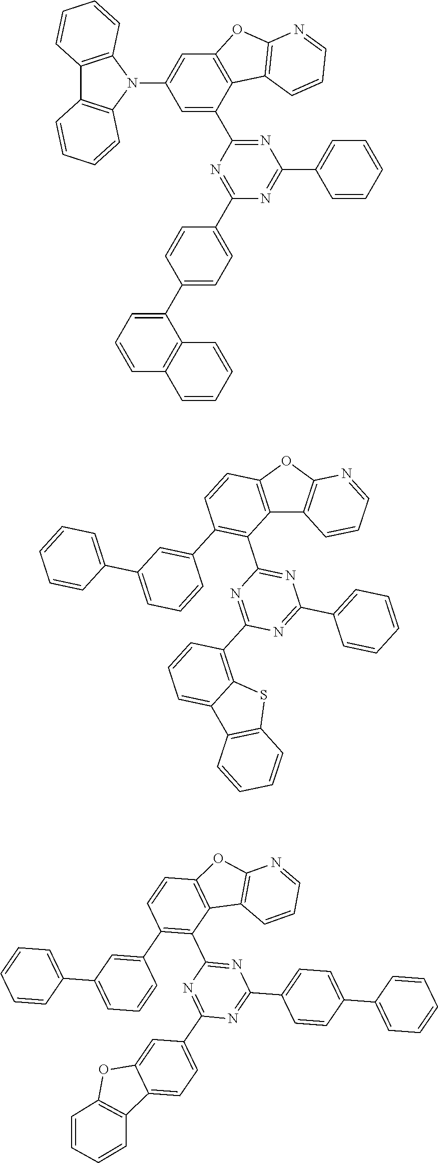 [SMILES: c1ccc(-c2nc(-c3ccc(-c4cccc5ccccc45)cc3)nc(-c3cc(-n4c5ccccc5c5ccccc54)cc4oc5ncccc5c34)n2)cc1 ; c1ccc(-c2cccc(-c3ccc4oc5ncccc5c4c3-c3nc(-c4ccccc4)nc(-c4cccc5c4sc4ccccc45)n3)c2)cc1 ; c1ccc(-c2ccc(-c3nc(-c4ccc5c(c4)oc4ccccc45)nc(-c4c(-c5cccc(-c6ccccc6)c5)ccc5oc6ncccc6c45)n3)cc2)cc1]

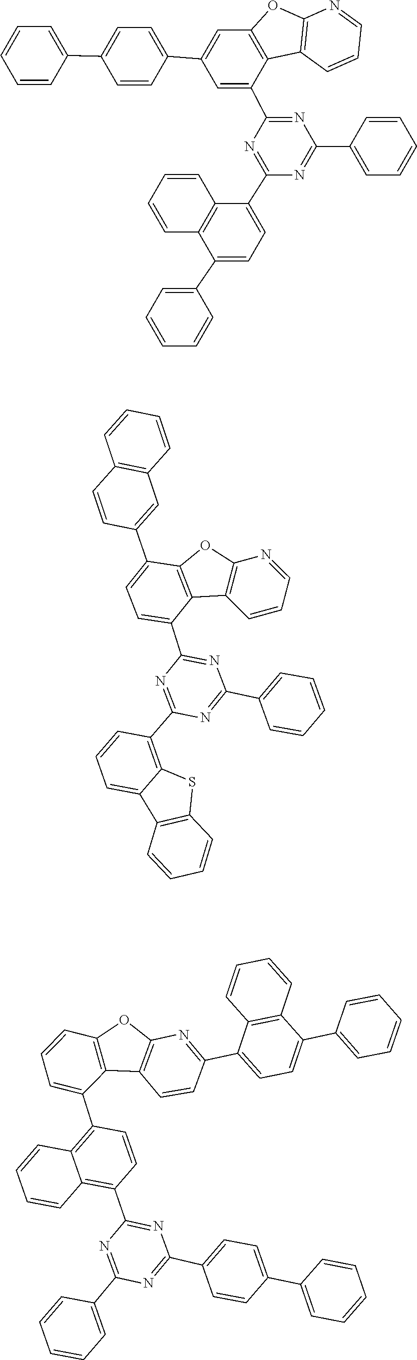 [SMILES: c1ccc(-c2ccc(-c3cc(-c4nc(-c5ccccc5)nc(-c5ccc(-c6ccccc6)c6ccccc56)n4)c4c(c3)oc3ncccc34)cc2)cc1 ; c1ccc(-c2nc(-c3cccc4c3sc3ccccc34)nc(-c3ccc(-c4ccc5ccccc5c4)c4oc5ncccc5c34)n2)cc1 ; c1ccc(-c2ccc(-c3nc(-c4ccccc4)nc(-c4ccc(-c5cccc6oc7nc(-c8ccc(-c9ccccc9)c9ccccc89)ccc7c56)c5ccccc45)n3)cc2)cc1]

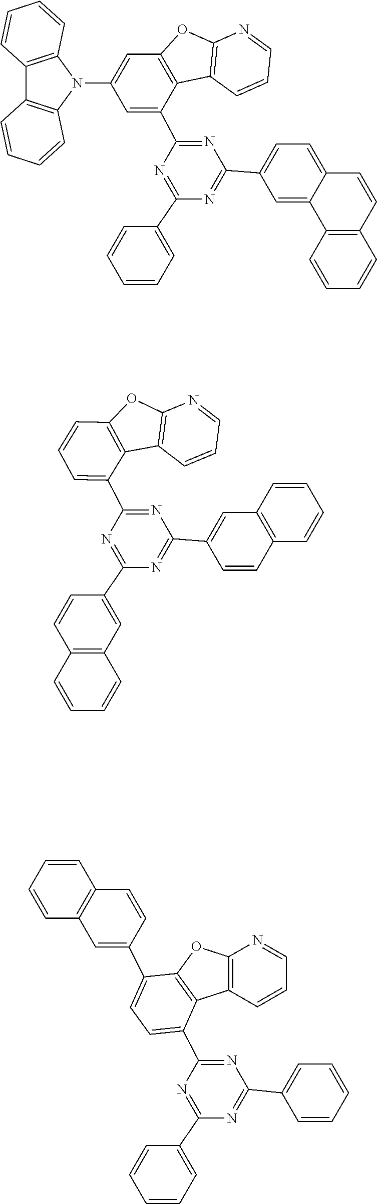 [SMILES: c1ccc(-c2nc(-c3ccc4ccc5ccccc5c4c3)nc(-c3cc(-n4c5ccccc5c5ccccc54)cc4oc5ncccc5c34)n2)cc1 ; c1ccc2cc(-c3nc(-c4ccc5ccccc5c4)nc(-c4cccc5oc6ncccc6c45)n3)ccc2c1 ; c1ccc(-c2nc(-c3ccccc3)nc(-c3ccc(-c4ccc5ccccc5c4)c4oc5ncccc5c34)n2)cc1]

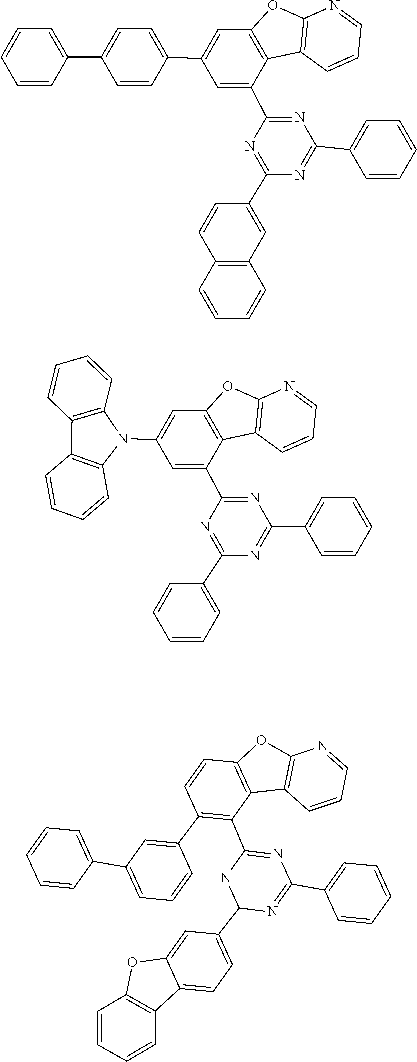

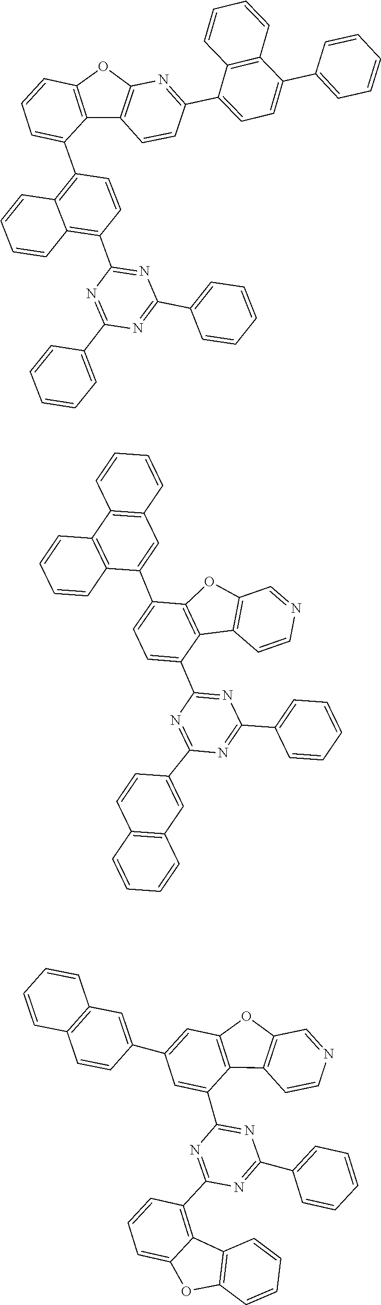 [SMILES: c1ccc(-c2nc(-c3ccccc3)nc(-c3ccc(-c4cccc5oc6nc(-c7ccc(-c8ccccc8)c8ccccc78)ccc6c45)c4ccccc34)n2)cc1 ; c1ccc(-c2nc(-c3ccc4ccccc4c3)nc(-c3ccc(-c4cc5ccccc5c5ccccc45)c4oc5cnccc5c34)n2)cc1 ; c1ccc(-c2nc(-c3cccc4oc5ccccc5c34)nc(-c3cc(-c4ccc5ccccc5c4)cc4oc5cnccc5c34)n2)cc1]

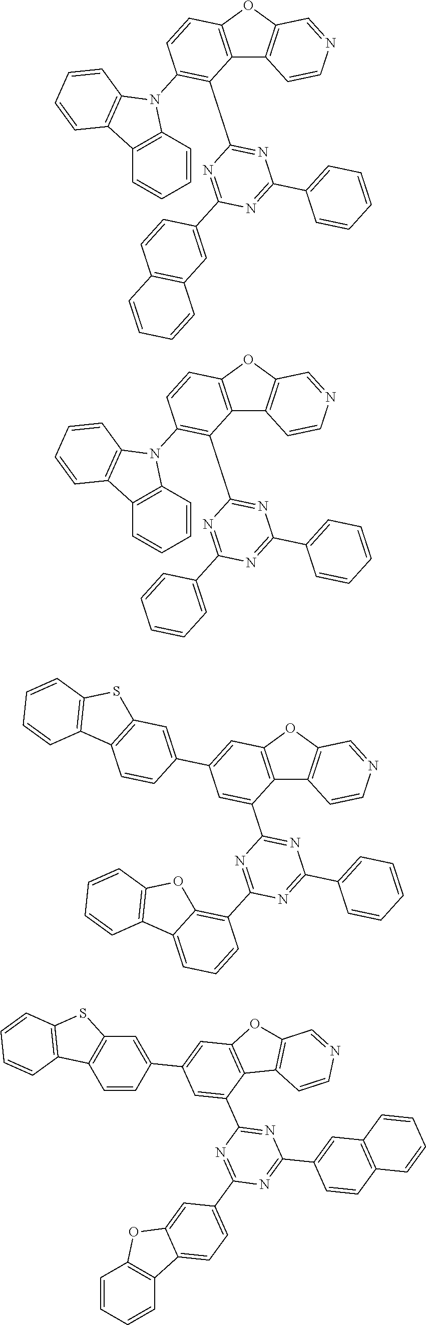

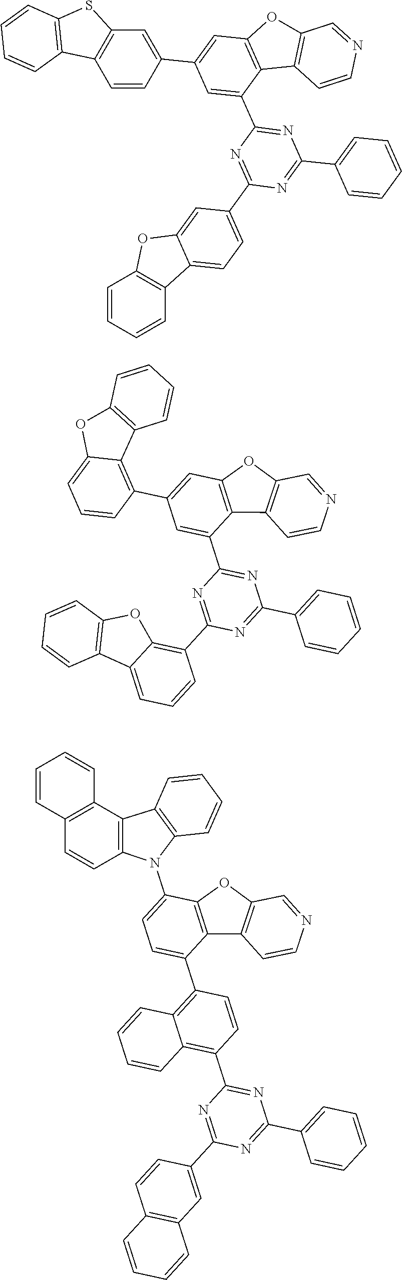

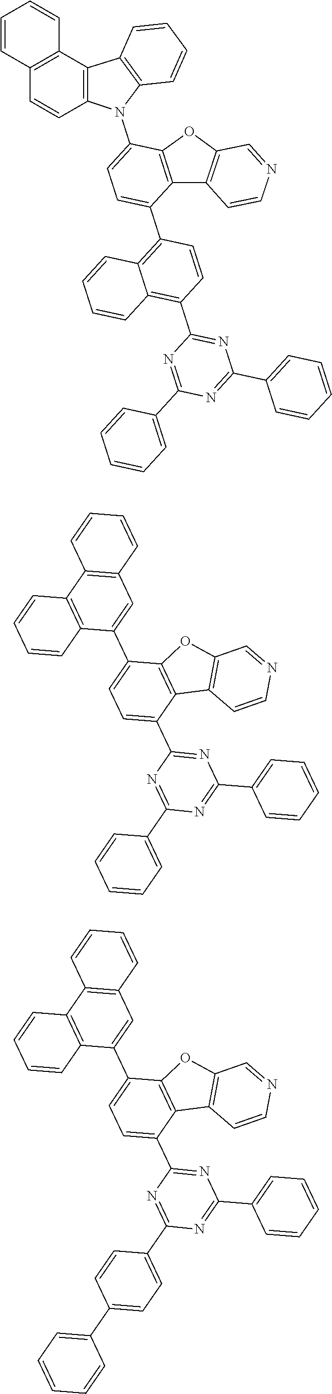

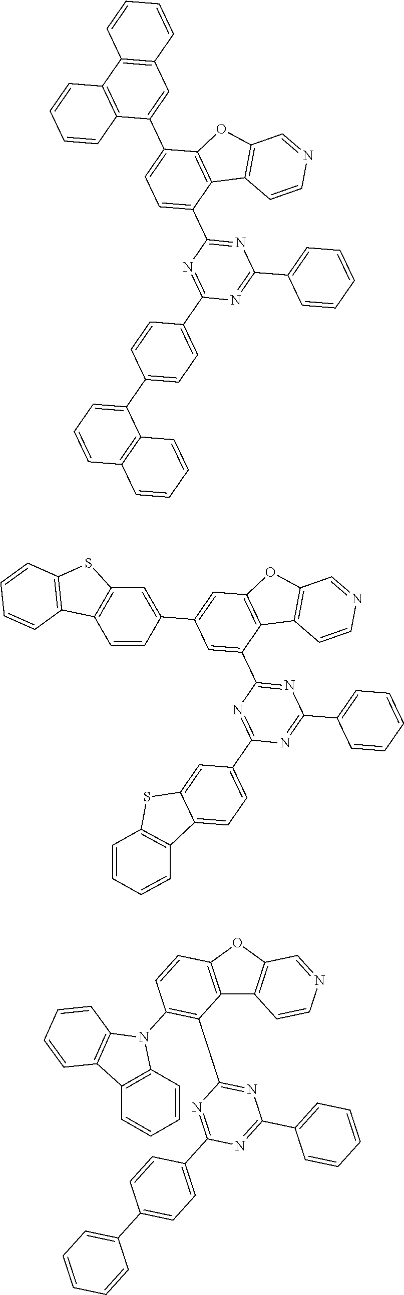 [SMILES: c1ccc(-c2nc(-c3ccc(-c4cccc5ccccc45)cc3)nc(-c3ccc(-c4cc5ccccc5c5ccccc45)c4oc5cnccc5c34)n2)cc1 ; c1ccc(-c2nc(-c3ccc4c(c3)sc3ccccc34)nc(-c3cc(-c4ccc5c(c4)sc4ccccc45)cc4oc5cnccc5c34)n2)cc1 ; c1ccc(-c2ccc(-c3nc(Cc4c(-n5c6ccccc6c6ccccc65)ccc5oc6cnccc6c45)nc(-c4ccccc4)n3)cc2)cc1]

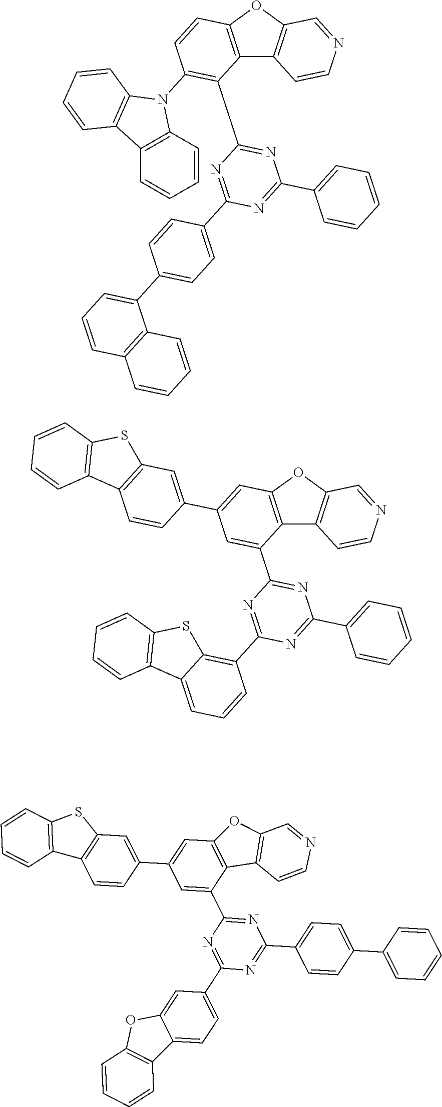 [SMILES: c1ccc(-c2nc(Cc3c(-n4c5ccccc5c5ccccc54)ccc4oc5cnccc5c34)nc(-c3ccc(-c4cccc5ccccc45)cc3)n2)cc1 ; c1ccc(-c2nc(-c3cccc4c3sc3ccccc34)nc(-c3cc(-c4ccc5c(c4)sc4ccccc45)cc4oc5cnccc5c34)n2)cc1 ; c1ccc(-c2ccc(-c3nc(-c4ccc5c(c4)oc4ccccc45)nc(-c4cc(-c5ccc6c(c5)sc5ccccc56)cc5oc6cnccc6c45)n3)cc2)cc1]

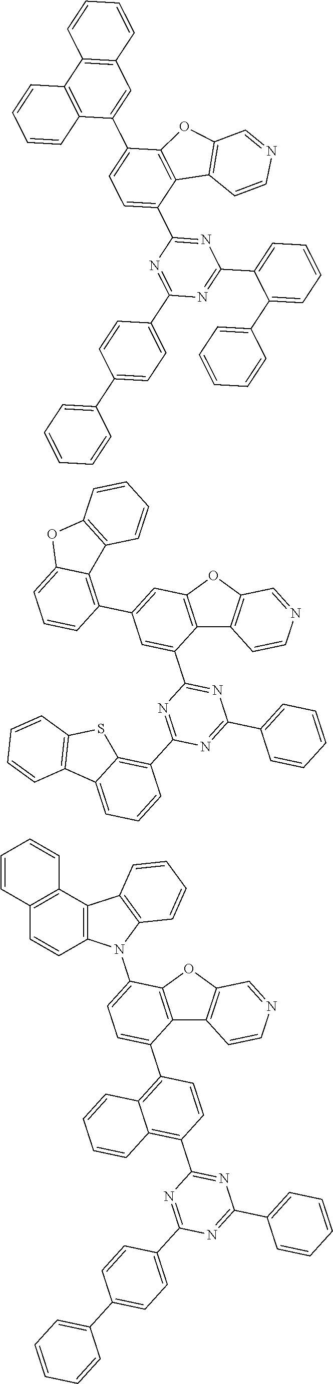

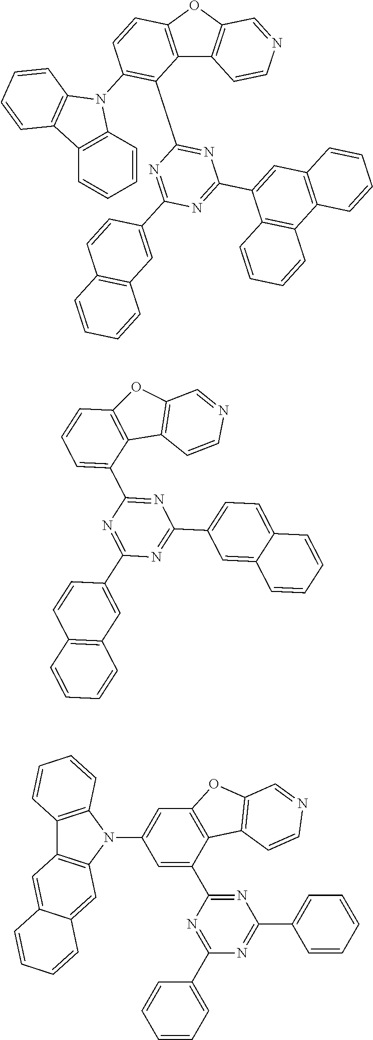 [SMILES: c1ccc2cc(-c3nc(Cc4c(-n5c6ccccc6c6ccccc65)ccc5oc6cnccc6c45)nc(-c4cc5ccccc5c5ccccc45)n3)ccc2c1 ; c1ccc2cc(-c3nc(-c4ccc5ccccc5c4)nc(-c4cccc5oc6cnccc6c45)n3)ccc2c1 ; c1ccc(-c2nc(-c3ccccc3)nc(-c3cc(-n4c5ccccc5c5cc6ccccc6cc54)cc4oc5cnccc5c34)n2)cc1]

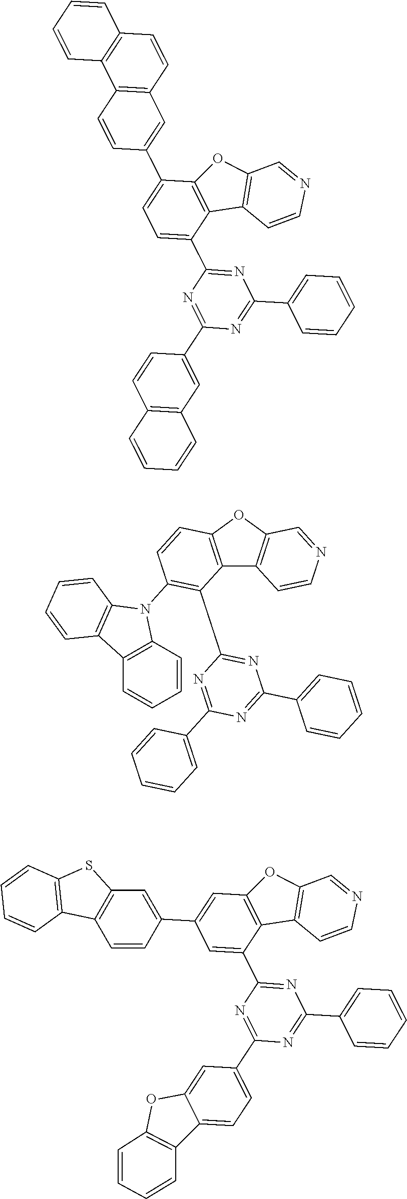 [SMILES: c1ccc(-c2nc(-c3ccc4ccccc4c3)nc(-c3ccc(-c4ccc5c(ccc6ccccc65)c4)c4oc5cnccc5c34)n2)cc1 ; c1ccc(-c2nc(Cc3c(-n4c5ccccc5c5ccccc54)ccc4oc5cnccc5c34)nc(-c3ccccc3)n2)cc1 ; c1ccc(-c2nc(-c3ccc4c(c3)oc3ccccc34)nc(-c3cc(-c4ccc5c(c4)sc4ccccc45)cc4oc5cnccc5c34)n2)cc1]

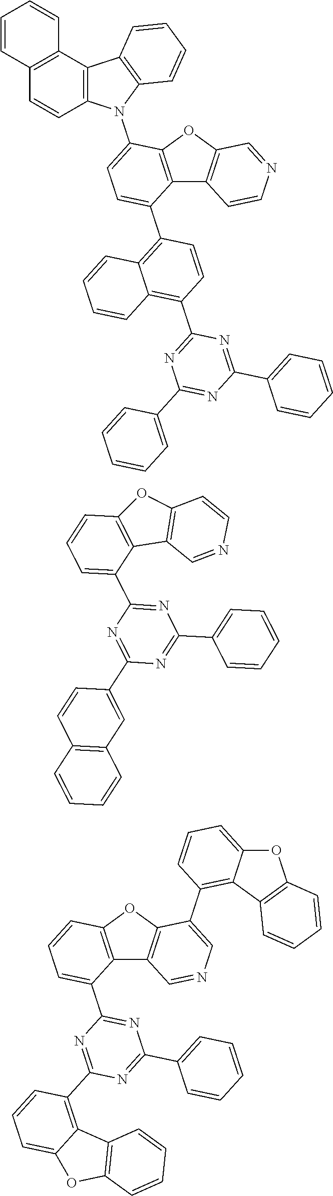

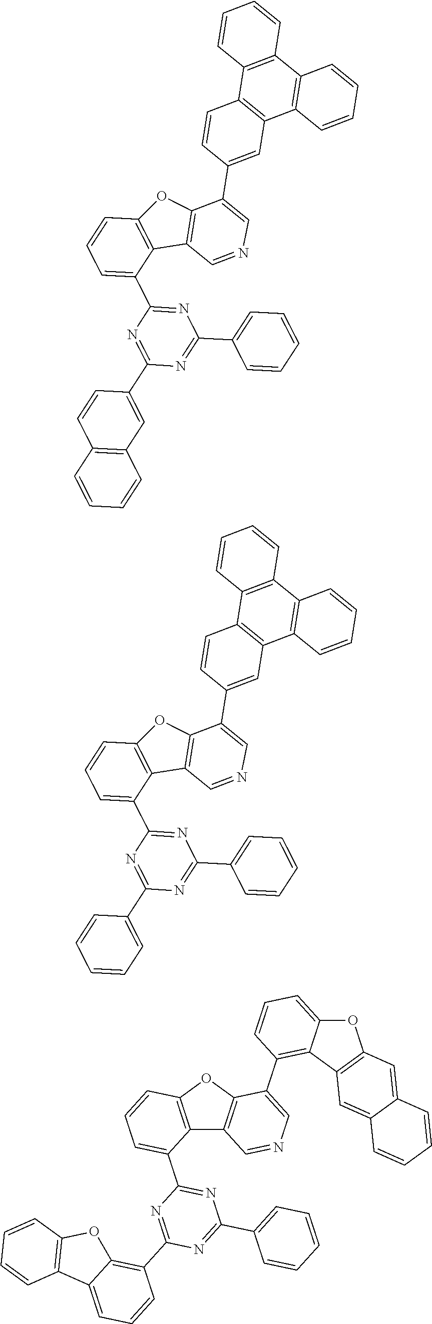 [SMILES: c1ccc(-c2nc(-c3ccc4ccccc4c3)nc(-c3cccc4oc5c(-c6ccc7c8ccccc8c8ccccc8c7c6)cncc5c34)n2)cc1 ; c1ccc(-c2nc(-c3ccccc3)nc(-c3cccc4oc5c(-c6ccc7c8ccccc8c8ccccc8c7c6)cncc5c34)n2)cc1 ; c1ccc(-c2nc(-c3cccc4c3oc3ccccc34)nc(-c3cccc4oc5c(-c6cccc7oc8cc9ccccc9cc8c67)cncc5c34)n2)cc1]

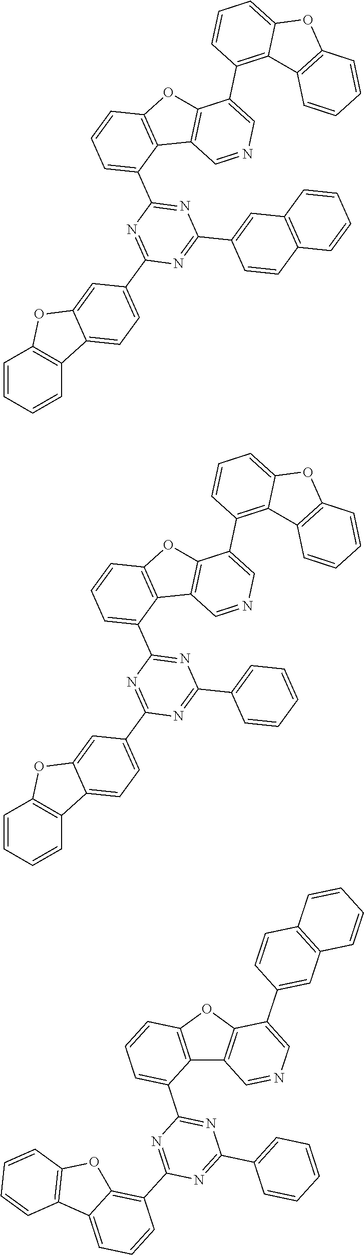 [SMILES: c1ccc2cc(-c3nc(-c4ccc5c(c4)oc4ccccc45)nc(-c4cccc5oc6c(-c7cccc8oc9ccccc9c78)cncc6c45)n3)ccc2c1 ; c1ccc(-c2nc(-c3ccc4c(c3)oc3ccccc34)nc(-c3cccc4oc5c(-c6cccc7oc8ccccc8c67)cncc5c34)n2)cc1 ; c1ccc(-c2nc(-c3cccc4c3oc3ccccc34)nc(-c3cccc4oc5c(-c6ccc7ccccc7c6)cncc5c34)n2)cc1]

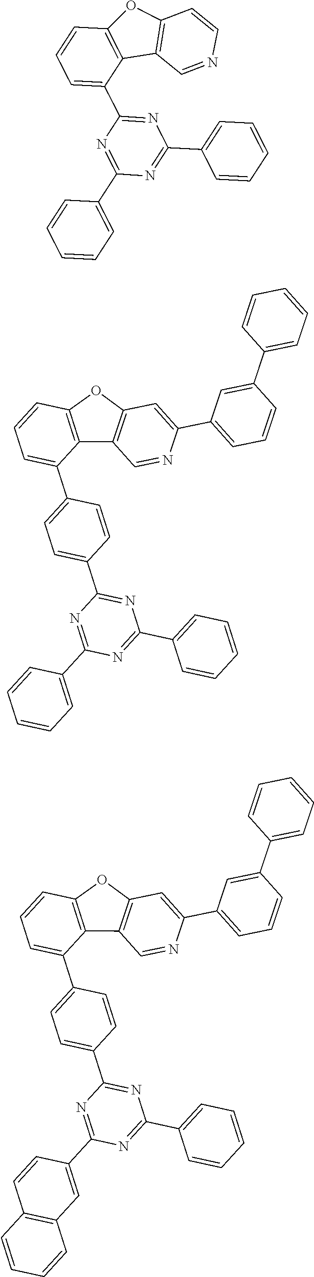 [SMILES: c1ccc(-c2nc(-c3ccccc3)nc(-c3cccc4oc5ccncc5c34)n2)cc1 ; c1ccc(-c2cccc(-c3cc4oc5cccc(-c6ccc(-c7nc(-c8ccccc8)nc(-c8ccccc8)n7)cc6)c5c4cn3)c2)cc1 ; c1ccc(-c2cccc(-c3cc4oc5cccc(-c6ccc(-c7nc(-c8ccccc8)nc(-c8ccc9ccccc9c8)n7)cc6)c5c4cn3)c2)cc1]

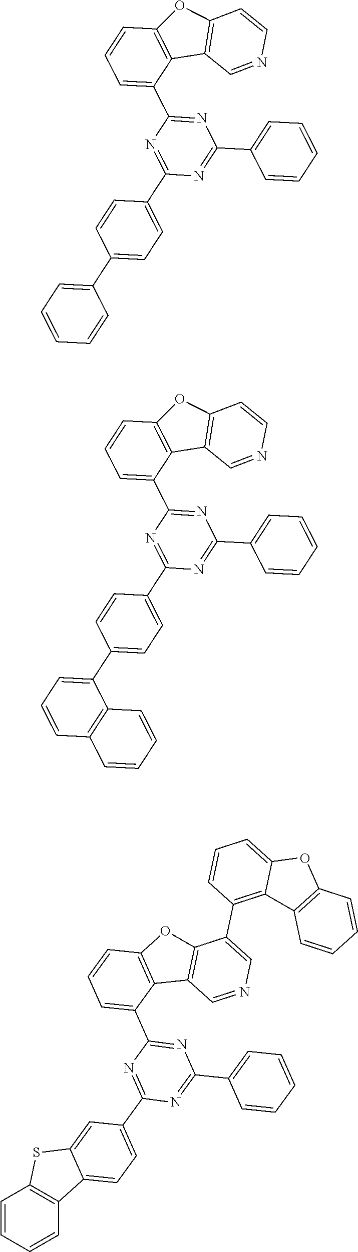 [SMILES: c1ccc(-c2ccc(-c3nc(-c4ccccc4)nc(-c4cccc5oc6ccncc6c45)n3)cc2)cc1 ; c1ccc(-c2nc(-c3ccc(-c4cccc5ccccc45)cc3)nc(-c3cccc4oc5ccncc5c34)n2)cc1 ; c1ccc(-c2nc(-c3ccc4c(c3)sc3ccccc34)nc(-c3cccc4oc5c(-c6cccc7oc8ccccc8c67)cncc5c34)n2)cc1]

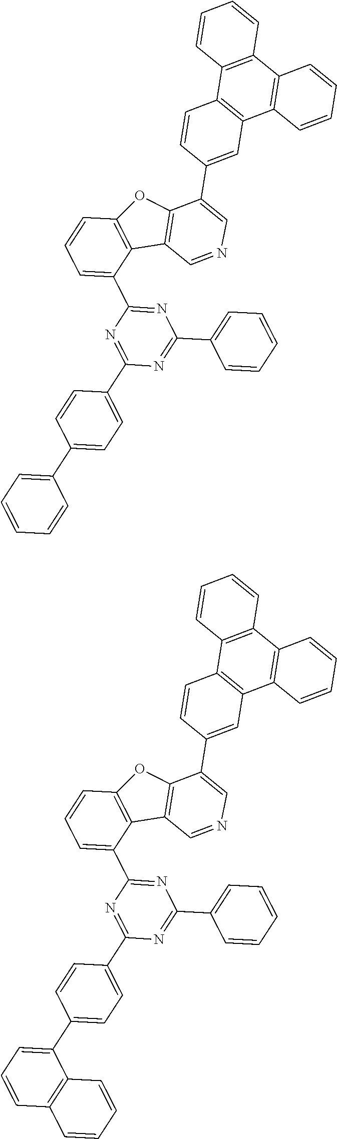 [SMILES: c1ccc(-c2ccc(-c3nc(-c4ccccc4)nc(-c4cccc5oc6c(-c7ccc8c9ccccc9c9ccccc9c8c7)cncc6c45)n3)cc2)cc1 ; c1ccc(-c2nc(-c3ccc(-c4cccc5ccccc45)cc3)nc(-c3cccc4oc5c(-c6ccc7c8ccccc8c8ccccc8c7c6)cncc5c34)n2)cc1]

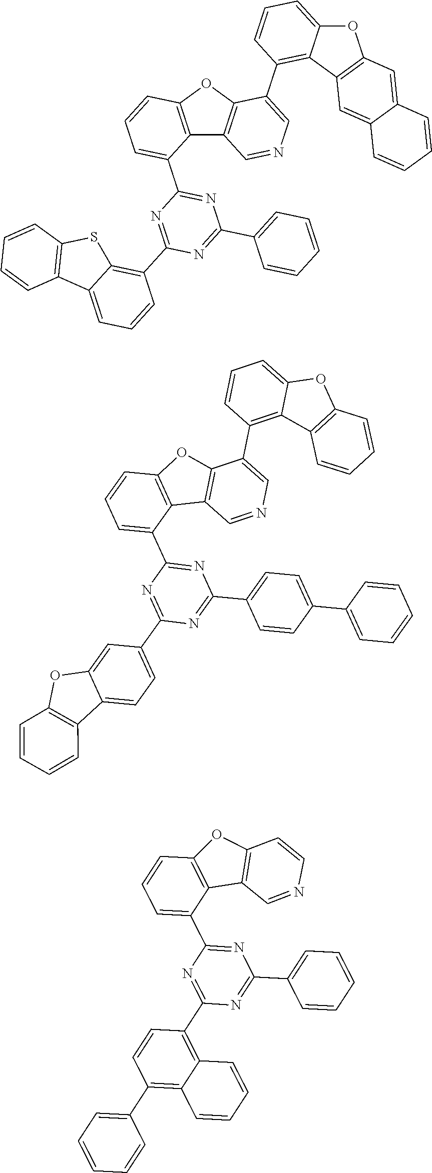 [SMILES: c1ccc(-c2nc(-c3cccc4c3sc3ccccc34)nc(-c3cccc4oc5c(-c6cccc7oc8cc9ccccc9cc8c67)cncc5c34)n2)cc1 ; c1ccc(-c2ccc(-c3nc(-c4ccc5c(c4)oc4ccccc45)nc(-c4cccc5oc6c(-c7cccc8oc9ccccc9c78)cncc6c45)n3)cc2)cc1 ; c1ccc(-c2nc(-c3ccc(-c4ccccc4)c4ccccc34)nc(-c3cccc4oc5ccncc5c34)n2)cc1]

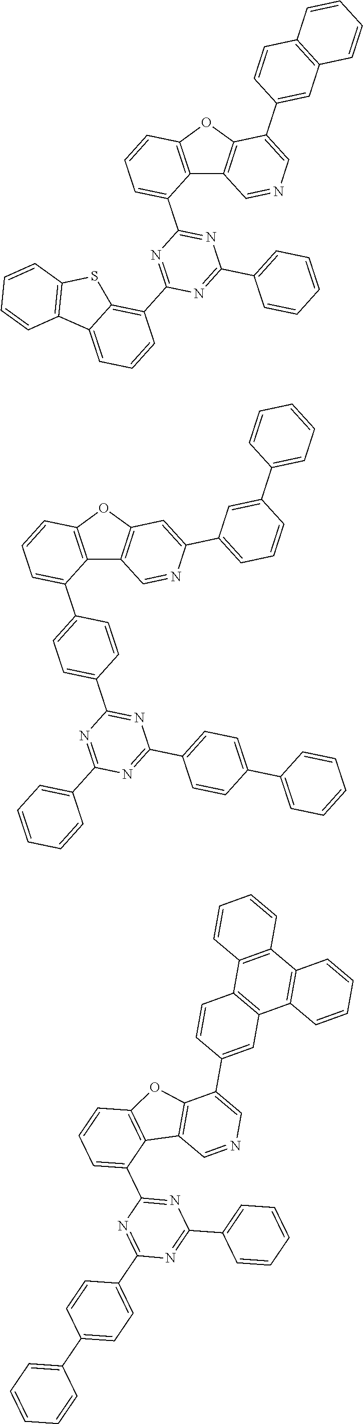 [SMILES: c1ccc(-c2nc(-c3cccc4c3sc3ccccc34)nc(-c3cccc4oc5c(-c6ccc7ccccc7c6)cncc5c34)n2)cc1 ; c1ccc(-c2ccc(-c3nc(-c4ccccc4)nc(-c4ccc(-c5cccc6oc7cc(-c8cccc(-c9ccccc9)c8)ncc7c56)cc4)n3)cc2)cc1 ; c1ccc(-c2ccc(-c3nc(-c4ccccc4)nc(-c4cccc5oc6c(-c7ccc8c9ccccc9c9ccccc9c8c7)cncc6c45)n3)cc2)cc1]

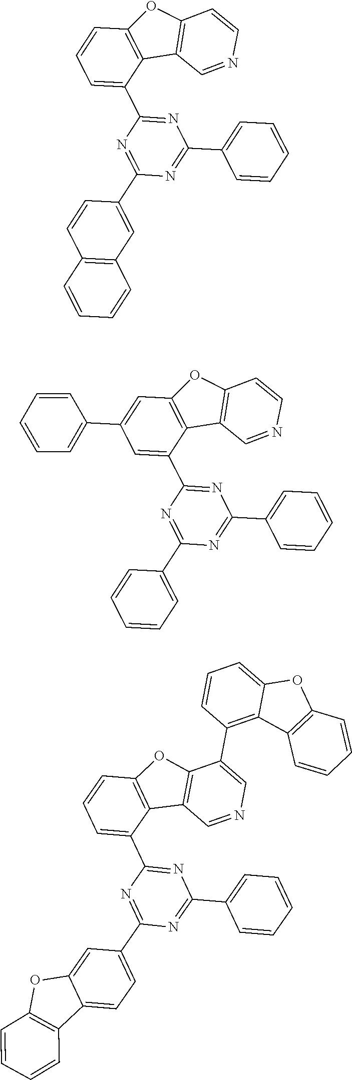 [SMILES: c1ccc(-c2nc(-c3ccc4ccccc4c3)nc(-c3cccc4oc5ccncc5c34)n2)cc1 ; c1ccc(-c2cc(-c3nc(-c4ccccc4)nc(-c4ccccc4)n3)c3c(c2)oc2ccncc23)cc1 ; c1ccc(-c2nc(-c3ccc4c(c3)oc3ccccc34)nc(-c3cccc4oc5c(-c6cccc7oc8ccccc8c67)cncc5c34)n2)cc1]

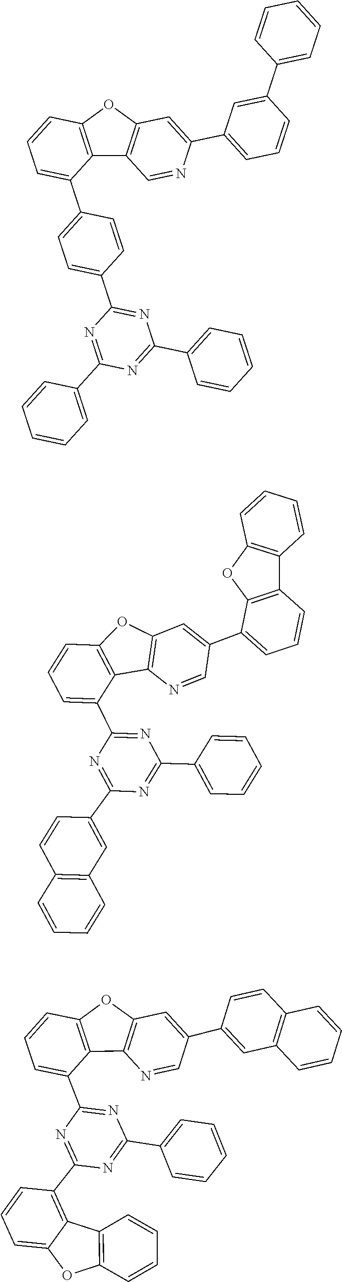 [SMILES: c1ccc(-c2cccc(-c3cc4oc5cccc(-c6ccc(-c7nc(-c8ccccc8)nc(-c8ccccc8)n7)cc6)c5c4cn3)c2)cc1 ; c1ccc(-c2nc(-c3ccc4ccccc4c3)nc(-c3cccc4oc5cc(-c6cccc7c6oc6ccccc67)cnc5c34)n2)cc1 ; c1ccc(-c2nc(-c3cccc4oc5ccccc5c34)nc(-c3cccc4oc5cc(-c6ccc7ccccc7c6)cnc5c34)n2)cc1]

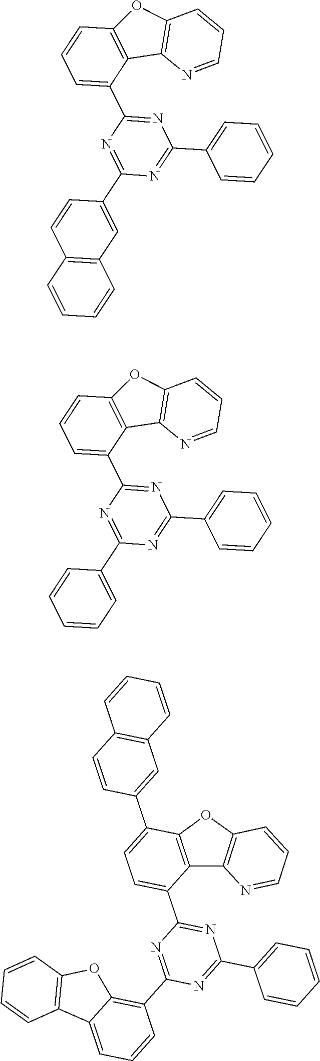 [SMILES: c1ccc(-c2nc(-c3ccc4ccccc4c3)nc(-c3cccc4oc5cccnc5c34)n2)cc1 ; c1ccc(-c2nc(-c3ccccc3)nc(-c3cccc4oc5cccnc5c34)n2)cc1 ; c1ccc(-c2nc(-c3cccc4c3oc3ccccc34)nc(-c3ccc(-c4ccc5ccccc5c4)c4oc5cccnc5c34)n2)cc1]

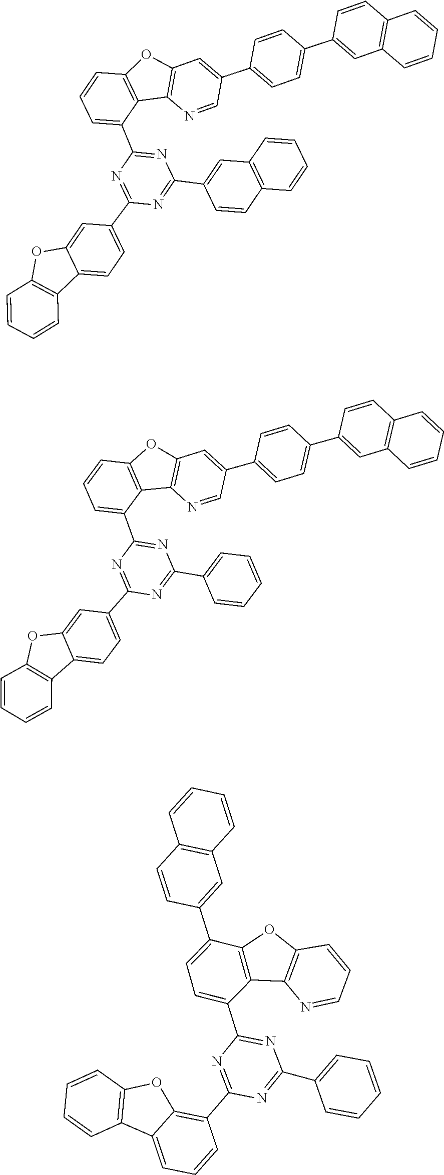 [SMILES: c1ccc2cc(-c3ccc(-c4cnc5c(c4)oc4cccc(-c6nc(-c7ccc8ccccc8c7)nc(-c7ccc8c(c7)oc7ccccc78)n6)c45)cc3)ccc2c1 ; c1ccc(-c2nc(-c3ccc4c(c3)oc3ccccc34)nc(-c3cccc4oc5cc(-c6ccc(-c7ccc8ccccc8c7)cc6)cnc5c34)n2)cc1 ; c1ccc(-c2nc(-c3cccc4c3oc3ccccc34)nc(-c3ccc(-c4ccc5ccccc5c4)c4oc5cccnc5c34)n2)cc1]

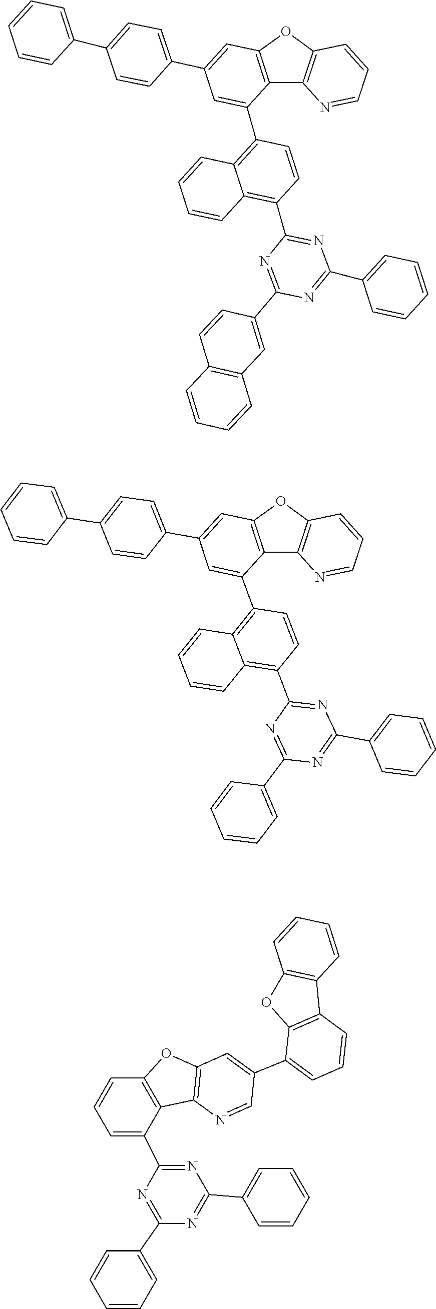 [SMILES: c1ccc(-c2ccc(-c3cc(-c4ccc(-c5nc(-c6ccccc6)nc(-c6ccc7ccccc7c6)n5)c5ccccc45)c4c(c3)oc3cccnc34)cc2)cc1 ; c1ccc(-c2ccc(-c3cc(-c4ccc(-c5nc(-c6ccccc6)nc(-c6ccccc6)n5)c5ccccc45)c4c(c3)oc3cccnc34)cc2)cc1 ; c1ccc(-c2nc(-c3ccccc3)nc(-c3cccc4oc5cc(-c6cccc7c6oc6ccccc67)cnc5c34)n2)cc1]

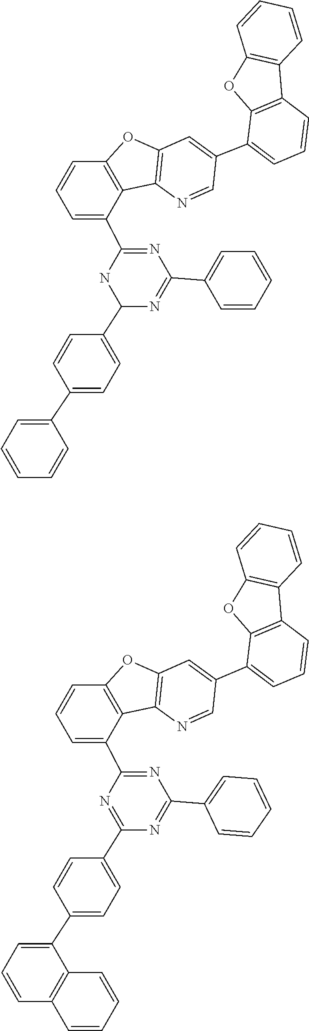

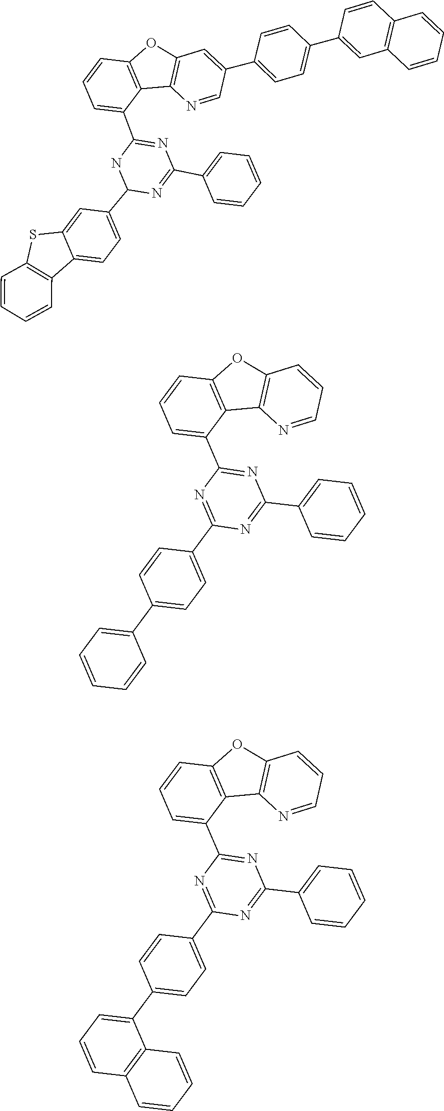

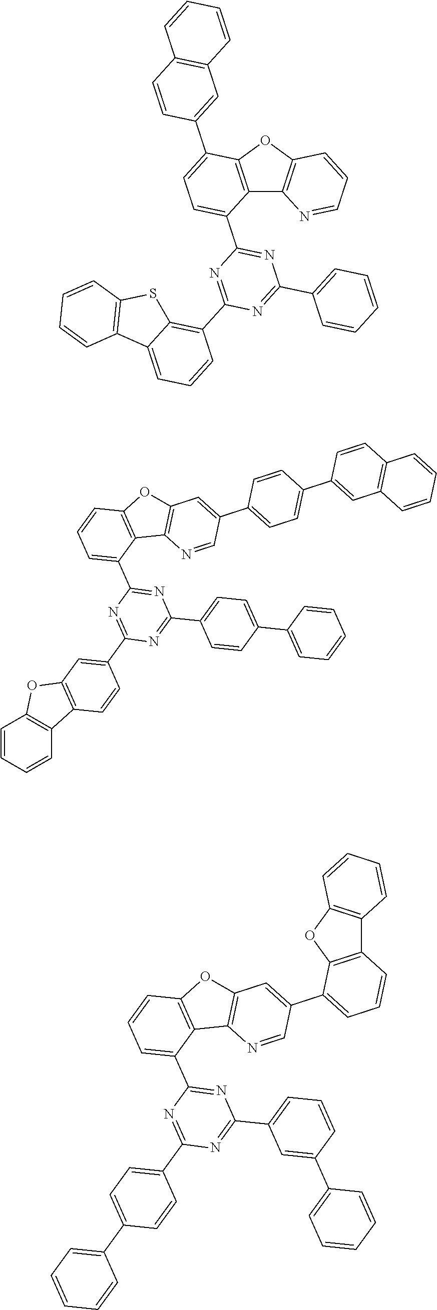 [SMILES: c1ccc(-c2nc(-c3cccc4c3sc3ccccc34)nc(-c3ccc(-c4ccc5ccccc5c4)c4oc5cccnc5c34)n2)cc1 ; c1ccc(-c2ccc(-c3nc(-c4ccc5c(c4)oc4ccccc45)nc(-c4cccc5oc6cc(-c7ccc(-c8ccc9ccccc9c8)cc7)cnc6c45)n3)cc2)cc1 ; c1ccc(-c2ccc(-c3nc(-c4cccc(-c5ccccc5)c4)nc(-c4cccc5oc6cc(-c7cccc8c7oc7ccccc78)cnc6c45)n3)cc2)cc1]

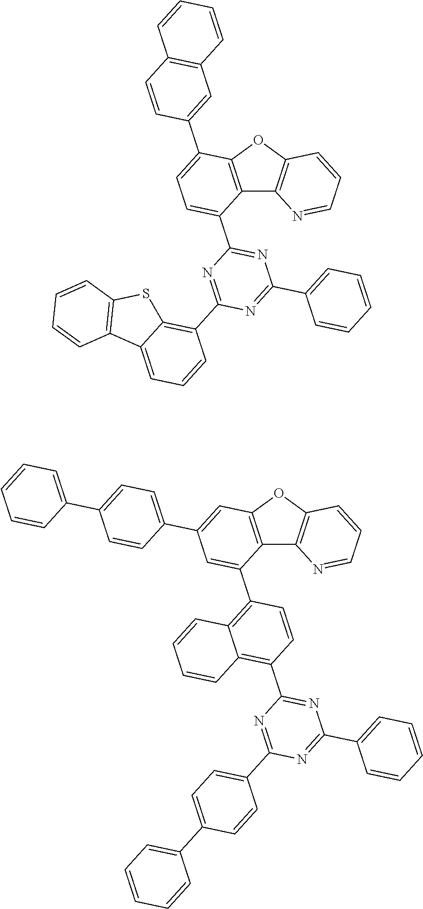 [SMILES: c1ccc(-c2nc(-c3cccc4c3sc3ccccc34)nc(-c3ccc(-c4ccc5ccccc5c4)c4oc5cccnc5c34)n2)cc1 ; c1ccc(-c2ccc(-c3cc(-c4ccc(-c5nc(-c6ccccc6)nc(-c6ccc(-c7ccccc7)cc6)n5)c5ccccc45)c4c(c3)oc3cccnc34)cc2)cc1]

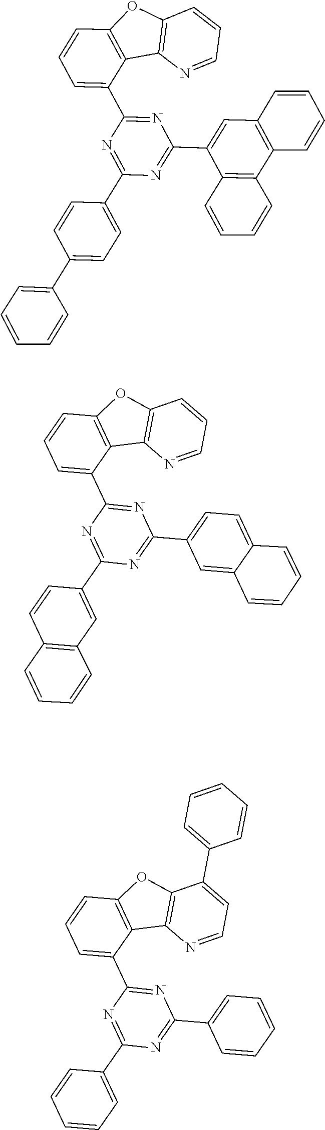 [SMILES: c1ccc(-c2ccc(-c3nc(-c4cc5ccccc5c5ccccc45)nc(-c4cccc5oc6cccnc6c45)n3)cc2)cc1 ; c1ccc2cc(-c3nc(-c4ccc5ccccc5c4)nc(-c4cccc5oc6cccnc6c45)n3)ccc2c1 ; c1ccc(-c2nc(-c3ccccc3)nc(-c3cccc4oc5c(-c6ccccc6)ccnc5c34)n2)cc1]

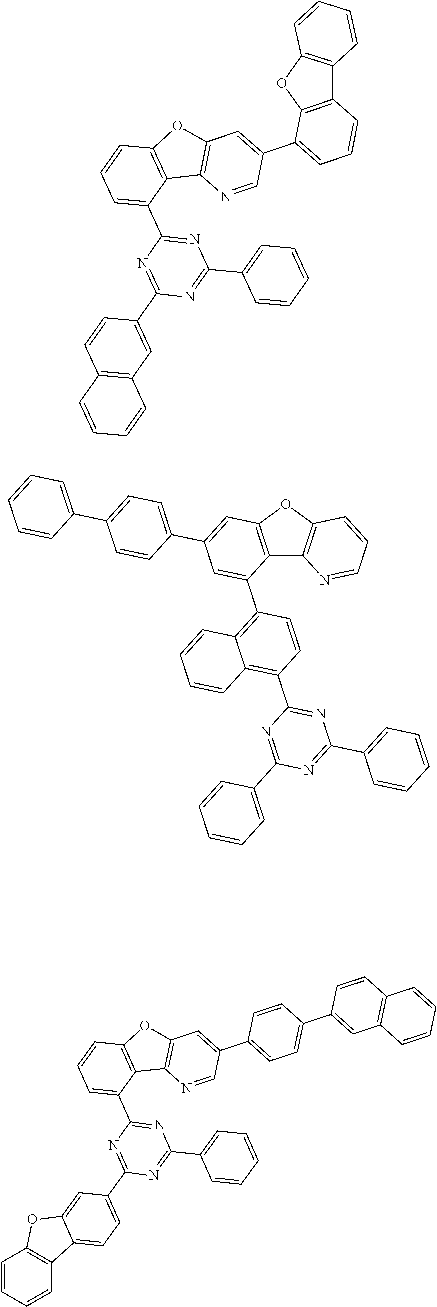 [SMILES: c1ccc(-c2nc(-c3ccc4ccccc4c3)nc(-c3cccc4oc5cc(-c6cccc7c6oc6ccccc67)cnc5c34)n2)cc1 ; c1ccc(-c2ccc(-c3cc(-c4ccc(-c5nc(-c6ccccc6)nc(-c6ccccc6)n5)c5ccccc45)c4c(c3)oc3cccnc34)cc2)cc1 ; c1ccc(-c2nc(-c3ccc4c(c3)oc3ccccc34)nc(-c3cccc4oc5cc(-c6ccc(-c7ccc8ccccc8c7)cc6)cnc5c34)n2)cc1]

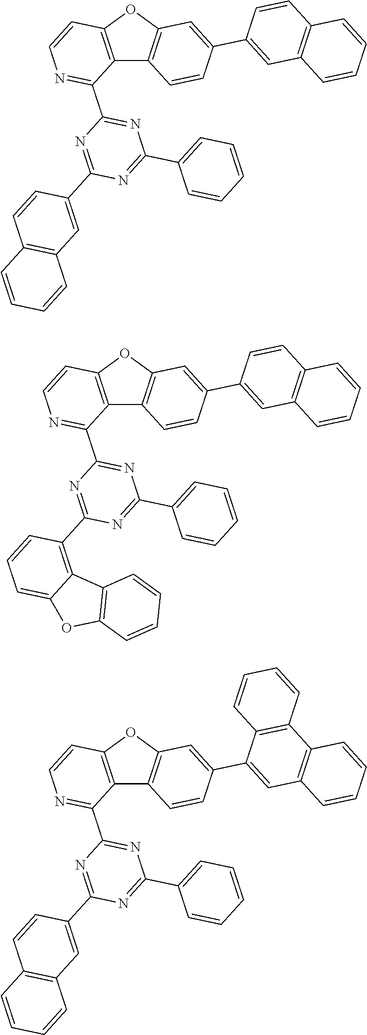 [SMILES: c1ccc(-c2nc(-c3ccc4ccccc4c3)nc(-c3nccc4oc5cc(-c6ccc7ccccc7c6)ccc5c34)n2)cc1 ; c1ccc(-c2nc(-c3cccc4oc5ccccc5c34)nc(-c3nccc4oc5cc(-c6ccc7ccccc7c6)ccc5c34)n2)cc1 ; c1ccc(-c2nc(-c3ccc4ccccc4c3)nc(-c3nccc4oc5cc(-c6cc7ccccc7c7ccccc67)ccc5c34)n2)cc1]

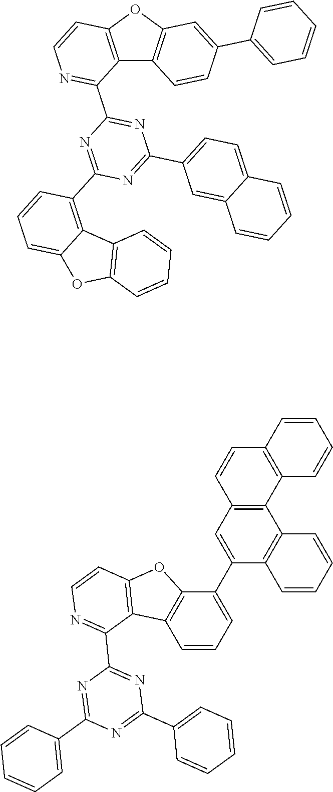 [SMILES: c1ccc(-c2ccc3c(c2)oc2ccnc(-c4nc(-c5ccc6ccccc6c5)nc(-c5cccc6oc7ccccc7c56)n4)c23)cc1 ; c1ccc(-c2nc(-c3ccccc3)nc(-c3nccc4oc5c(-c6cc7ccc8ccccc8c7c7ccccc67)cccc5c34)n2)cc1]

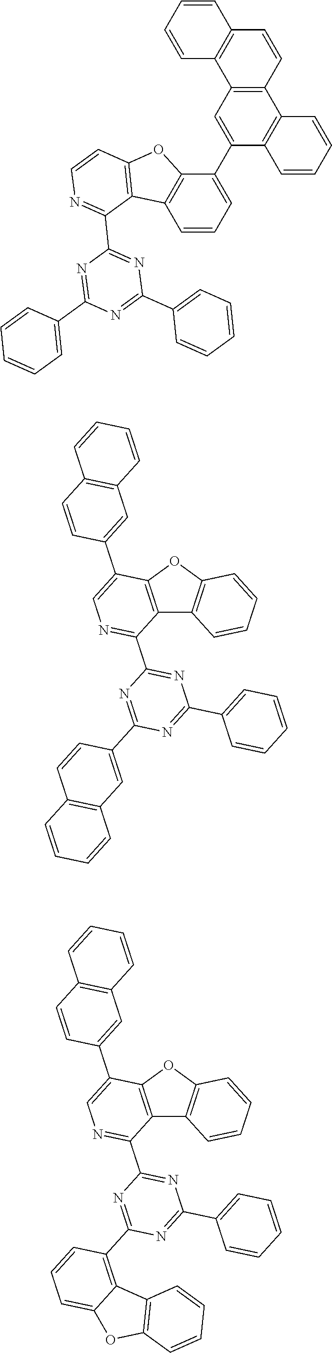 [SMILES: c1ccc(-c2nc(-c3ccccc3)nc(-c3nccc4oc5c(-c6cc7c8ccccc8ccc7c7ccccc67)cccc5c34)n2)cc1 ; c1ccc(-c2nc(-c3ccc4ccccc4c3)nc(-c3ncc(-c4ccc5ccccc5c4)c4oc5ccccc5c34)n2)cc1 ; c1ccc(-c2nc(-c3cccc4oc5ccccc5c34)nc(-c3ncc(-c4ccc5ccccc5c4)c4oc5ccccc5c34)n2)cc1]

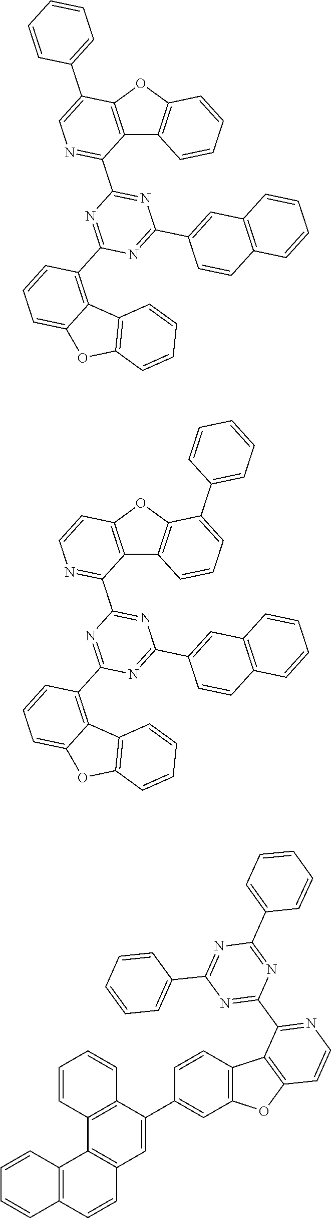 [SMILES: c1ccc(-c2cnc(-c3nc(-c4ccc5ccccc5c4)nc(-c4cccc5oc6ccccc6c45)n3)c3c2oc2ccccc23)cc1 ; c1ccc(-c2cccc3c2oc2ccnc(-c4nc(-c5ccc6ccccc6c5)nc(-c5cccc6oc7ccccc7c56)n4)c23)cc1 ; c1ccc(-c2nc(-c3ccccc3)nc(-c3nccc4oc5cc(-c6cc7ccc8ccccc8c7c7ccccc67)ccc5c34)n2)cc1]

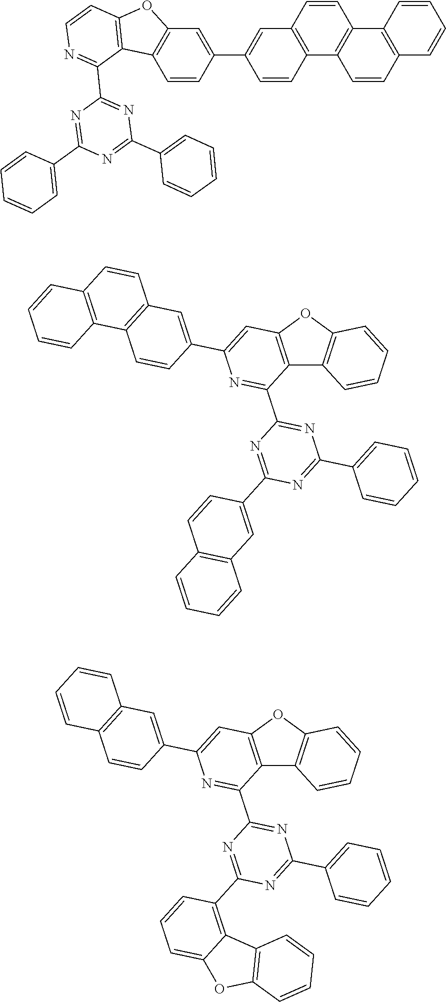 [SMILES: c1ccc(-c2nc(-c3ccccc3)nc(-c3nccc4oc5cc(-c6ccc7c(ccc8c9ccccc9ccc78)c6)ccc5c34)n2)cc1 ; c1ccc(-c2nc(-c3ccc4ccccc4c3)nc(-c3nc(-c4ccc5c(ccc6ccccc65)c4)cc4oc5ccccc5c34)n2)cc1 ; c1ccc(-c2nc(-c3cccc4oc5ccccc5c34)nc(-c3nc(-c4ccc5ccccc5c4)cc4oc5ccccc5c34)n2)cc1]

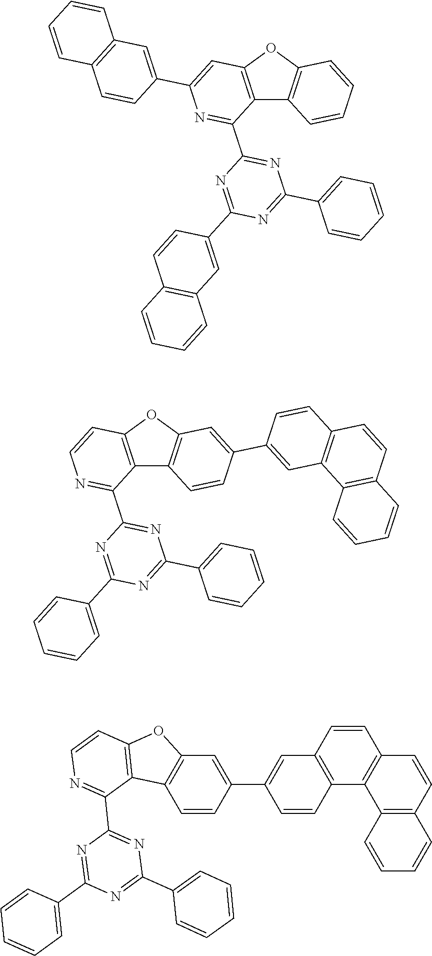 [SMILES: c1ccc(-c2nc(-c3ccc4ccccc4c3)nc(-c3nc(-c4ccc5ccccc5c4)cc4oc5ccccc5c34)n2)cc1 ; c1ccc(-c2nc(-c3ccccc3)nc(-c3nccc4oc5cc(-c6ccc7ccc8ccccc8c7c6)ccc5c34)n2)cc1 ; c1ccc(-c2nc(-c3ccccc3)nc(-c3nccc4oc5cc(-c6ccc7c(ccc8ccc9ccccc9c87)c6)ccc5c34)n2)cc1]

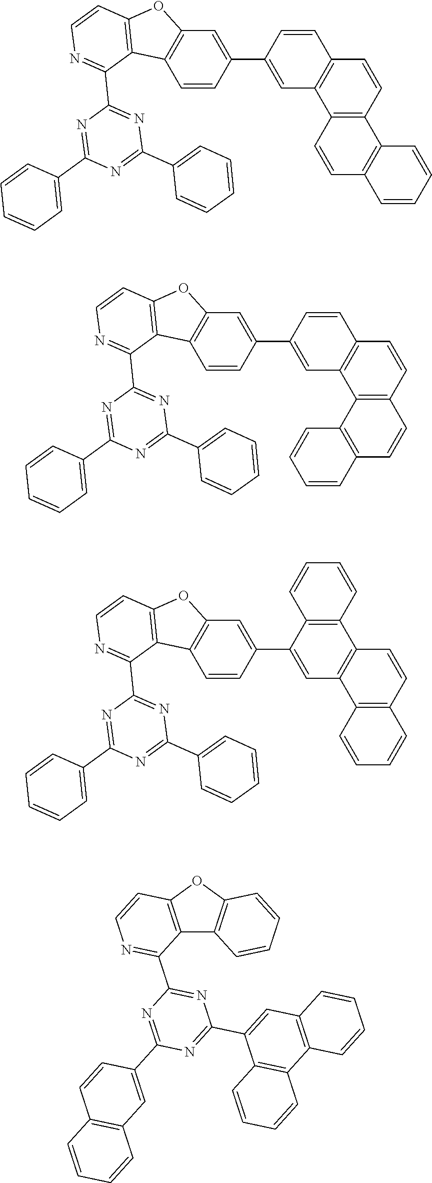 [SMILES: c1ccc(-c2nc(-c3ccccc3)nc(-c3nccc4oc5cc(-c6ccc7ccc8c9ccccc9ccc8c7c6)ccc5c34)n2)cc1 ; c1ccc(-c2nc(-c3ccccc3)nc(-c3nccc4oc5cc(-c6ccc7ccc8ccc9ccccc9c8c7c6)ccc5c34)n2)cc1 ; c1ccc(-c2nc(-c3ccccc3)nc(-c3nccc4oc5cc(-c6cc7c8ccccc8ccc7c7ccccc67)ccc5c34)n2)cc1 ; c1ccc2cc(-c3nc(-c4cc5ccccc5c5ccccc45)nc(-c4nccc5oc6ccccc6c45)n3)ccc2c1]

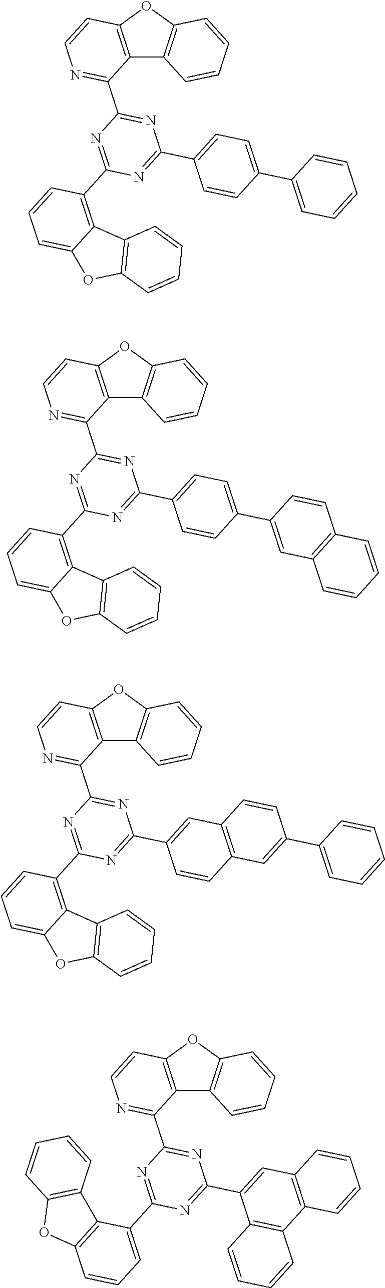 [SMILES: c1ccc(-c2ccc(-c3nc(-c4cccc5oc6ccccc6c45)nc(-c4nccc5oc6ccccc6c45)n3)cc2)cc1 ; c1ccc2cc(-c3ccc(-c4nc(-c5cccc6oc7ccccc7c56)nc(-c5nccc6oc7ccccc7c56)n4)cc3)ccc2c1 ; c1ccc(-c2ccc3cc(-c4nc(-c5cccc6oc7ccccc7c56)nc(-c5nccc6oc7ccccc7c56)n4)ccc3c2)cc1 ; c1ccc2c(c1)cc(-c1nc(-c3cccc4oc5ccccc5c34)nc(-c3nccc4oc5ccccc5c34)n1)c1ccccc12]

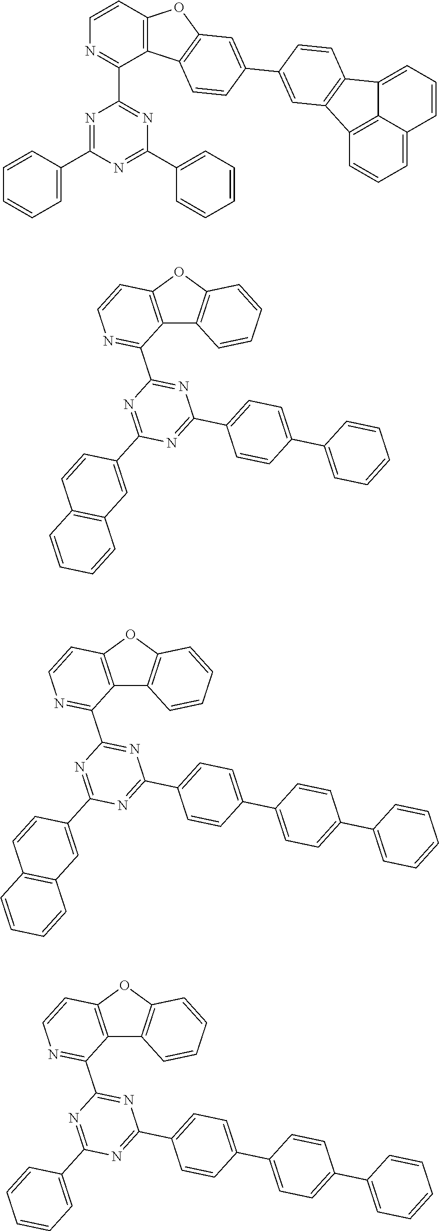 [SMILES: c1ccc(-c2nc(-c3ccccc3)nc(-c3nccc4oc5cc(-c6ccc7c(c6)-c6cccc8cccc-7c68)ccc5c34)n2)cc1 ; c1ccc(-c2ccc(-c3nc(-c4ccc5ccccc5c4)nc(-c4nccc5oc6ccccc6c45)n3)cc2)cc1 ; c1ccc(-c2ccc(-c3ccc(-c4nc(-c5ccc6ccccc6c5)nc(-c5nccc6oc7ccccc7c56)n4)cc3)cc2)cc1 ; c1ccc(-c2ccc(-c3ccc(-c4nc(-c5ccccc5)nc(-c5nccc6oc7ccccc7c56)n4)cc3)cc2)cc1]

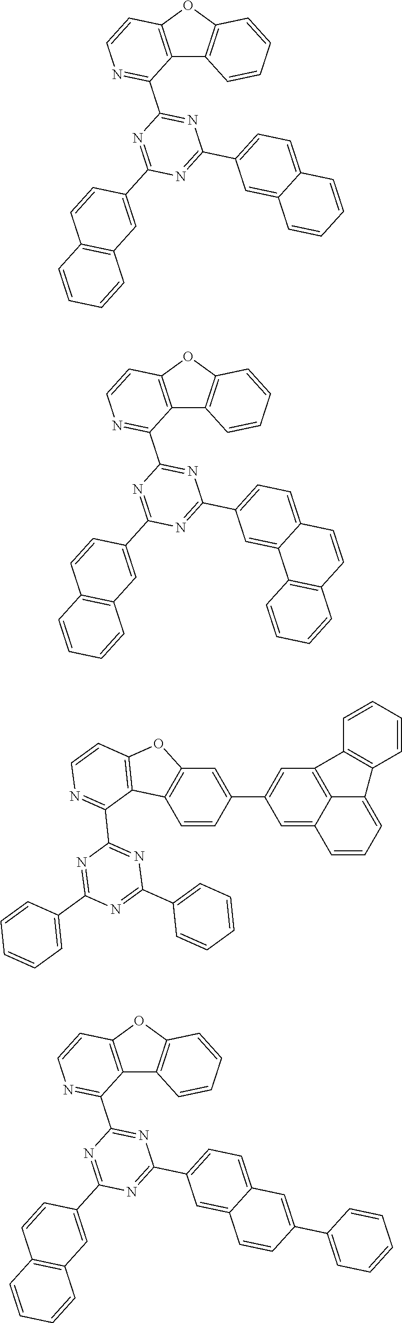 [SMILES: c1ccc2cc(-c3nc(-c4ccc5ccccc5c4)nc(-c4nccc5oc6ccccc6c45)n3)ccc2c1 ; c1ccc2cc(-c3nc(-c4ccc5ccc6ccccc6c5c4)nc(-c4nccc5oc6ccccc6c45)n3)ccc2c1 ; c1ccc(-c2nc(-c3ccccc3)nc(-c3nccc4oc5cc(-c6cc7c8c(cccc8c6)-c6ccccc6-7)ccc5c34)n2)cc1 ; c1ccc(-c2ccc3cc(-c4nc(-c5ccc6ccccc6c5)nc(-c5nccc6oc7ccccc7c56)n4)ccc3c2)cc1]

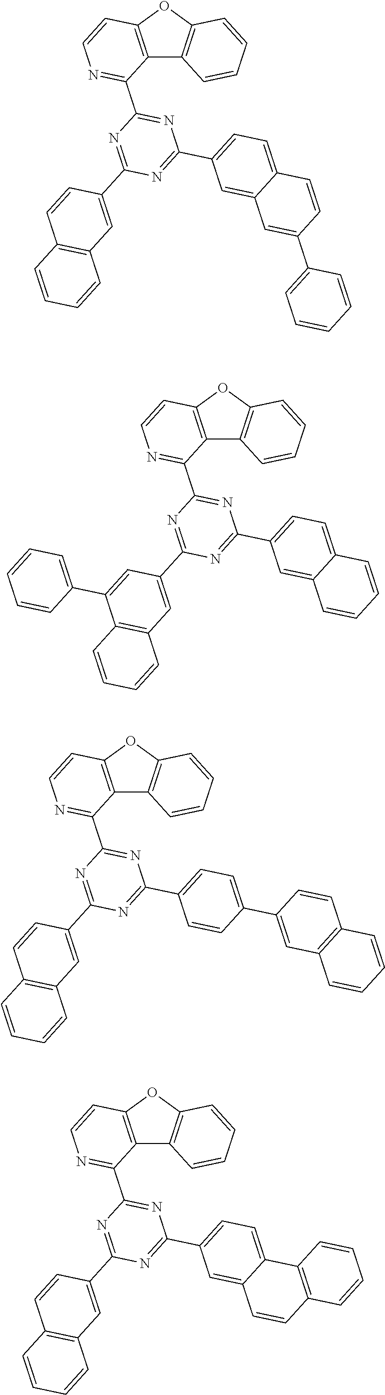 [SMILES: c1ccc(-c2ccc3ccc(-c4nc(-c5ccc6ccccc6c5)nc(-c5nccc6oc7ccccc7c56)n4)cc3c2)cc1 ; c1ccc(-c2cc(-c3nc(-c4ccc5ccccc5c4)nc(-c4nccc5oc6ccccc6c45)n3)cc3ccccc23)cc1 ; c1ccc2cc(-c3ccc(-c4nc(-c5ccc6ccccc6c5)nc(-c5nccc6oc7ccccc7c56)n4)cc3)ccc2c1 ; c1ccc2cc(-c3nc(-c4ccc5c(ccc6ccccc65)c4)nc(-c4nccc5oc6ccccc6c45)n3)ccc2c1]

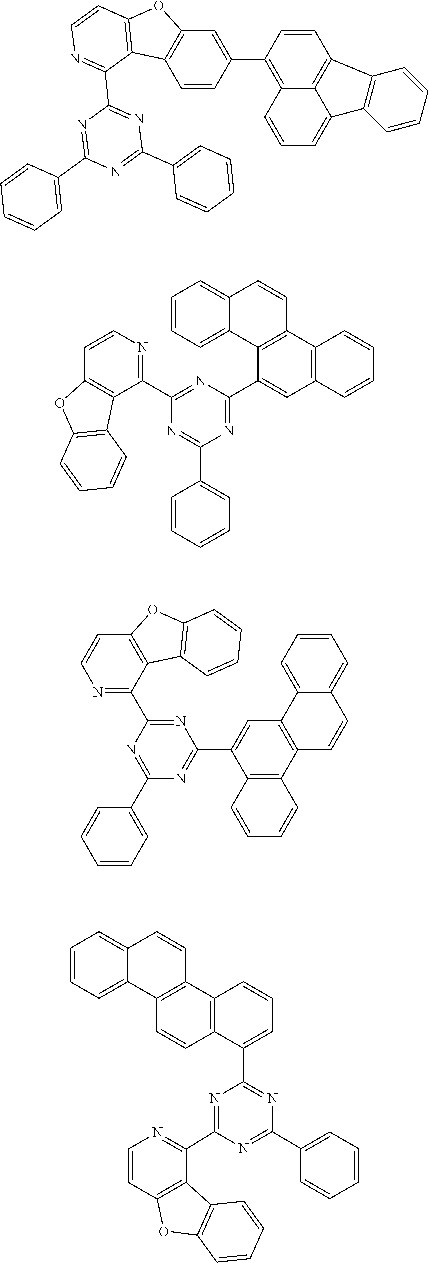 [SMILES: c1ccc(-c2nc(-c3ccccc3)nc(-c3nccc4oc5cc(-c6ccc7c8c(cccc68)-c6ccccc6-7)ccc5c34)n2)cc1 ; c1ccc(-c2nc(-c3nccc4oc5ccccc5c34)nc(-c3cc4ccccc4c4ccc5ccccc5c34)n2)cc1 ; c1ccc(-c2nc(-c3cc4c5ccccc5ccc4c4ccccc34)nc(-c3nccc4oc5ccccc5c34)n2)cc1 ; c1ccc(-c2nc(-c3cccc4c3ccc3c5ccccc5ccc43)nc(-c3nccc4oc5ccccc5c34)n2)cc1]

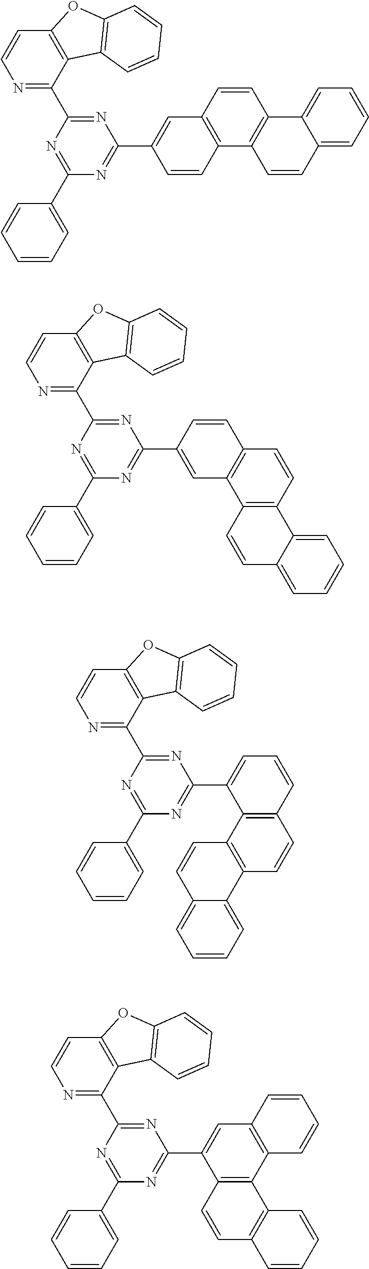 [SMILES: c1ccc(-c2nc(-c3ccc4c(ccc5c6ccccc6ccc45)c3)nc(-c3nccc4oc5ccccc5c34)n2)cc1 ; c1ccc(-c2nc(-c3ccc4ccc5c6ccccc6ccc5c4c3)nc(-c3nccc4oc5ccccc5c34)n2)cc1 ; c1ccc(-c2nc(-c3cccc4ccc5c6ccccc6ccc5c34)nc(-c3nccc4oc5ccccc5c34)n2)cc1 ; c1ccc(-c2nc(-c3cc4ccccc4c4c3ccc3ccccc34)nc(-c3nccc4oc5ccccc5c34)n2)cc1]

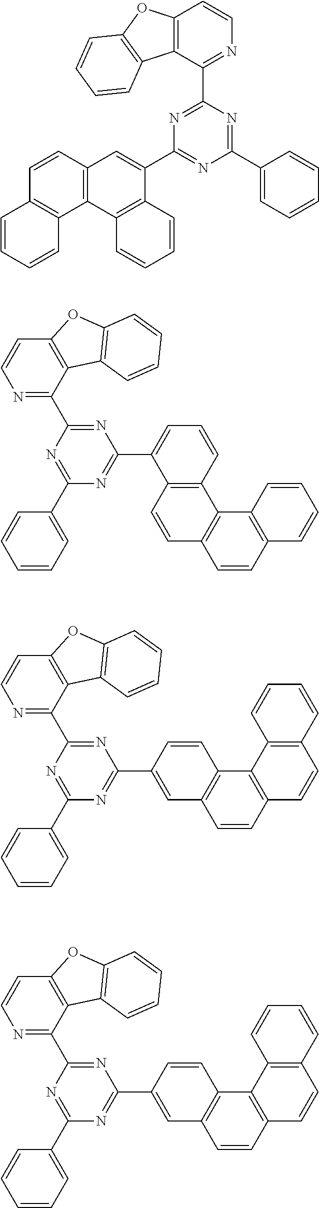 [SMILES: c1ccc(-c2nc(-c3cc4ccc5ccccc5c4c4ccccc34)nc(-c3nccc4oc5ccccc5c34)n2)cc1 ; c1ccc(-c2nc(-c3cccc4c3ccc3ccc5ccccc5c34)nc(-c3nccc4oc5ccccc5c34)n2)cc1 ; c1ccc(-c2nc(-c3ccc4c(ccc5ccc6ccccc6c54)c3)nc(-c3nccc4oc5ccccc5c34)n2)cc1 ; c1ccc(-c2nc(-c3ccc4c(ccc5ccc6ccccc6c54)c3)nc(-c3nccc4oc5ccccc5c34)n2)cc1]

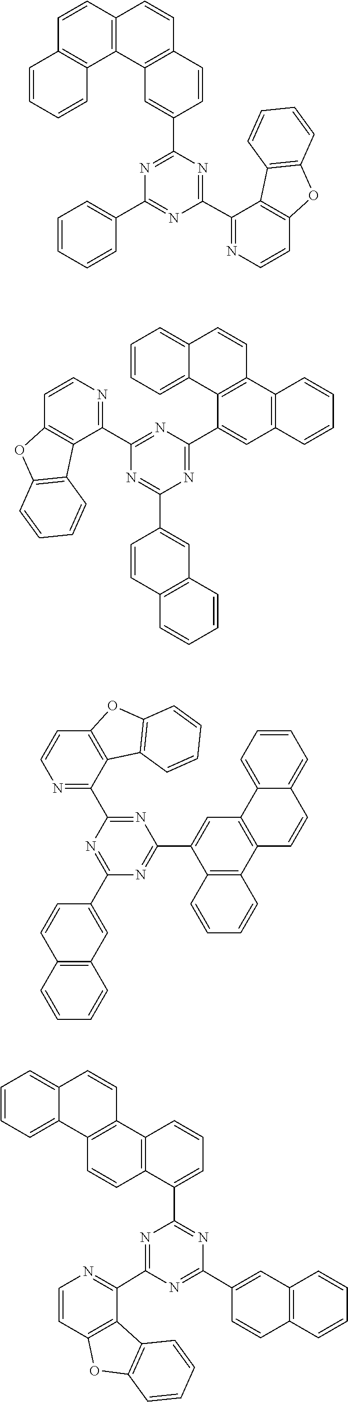 [SMILES: c1ccc(-c2nc(-c3ccc4ccc5ccc6ccccc6c5c4c3)nc(-c3nccc4oc5ccccc5c34)n2)cc1 ; c1ccc2cc(-c3nc(-c4nccc5oc6ccccc6c45)nc(-c4cc5ccccc5c5ccc6ccccc6c45)n3)ccc2c1 ; c1ccc2cc(-c3nc(-c4cc5c6ccccc6ccc5c5ccccc45)nc(-c4nccc5oc6ccccc6c45)n3)ccc2c1 ; c1ccc2cc(-c3nc(-c4cccc5c4ccc4c6ccccc6ccc54)nc(-c4nccc5oc6ccccc6c45)n3)ccc2c1]

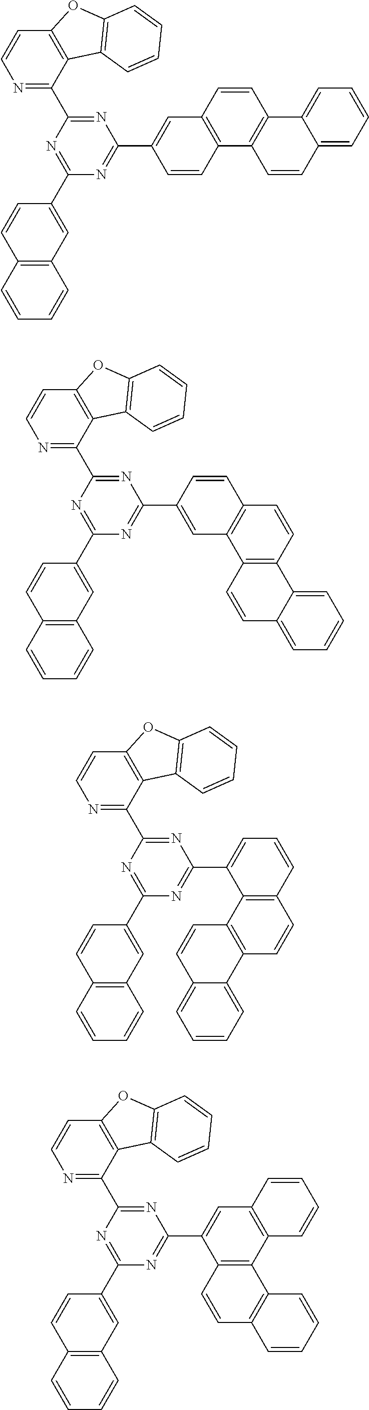 [SMILES: c1ccc2cc(-c3nc(-c4ccc5c(ccc6c7ccccc7ccc56)c4)nc(-c4nccc5oc6ccccc6c45)n3)ccc2c1 ; c1ccc2cc(-c3nc(-c4ccc5ccc6c7ccccc7ccc6c5c4)nc(-c4nccc5oc6ccccc6c45)n3)ccc2c1 ; c1ccc2cc(-c3nc(-c4cccc5ccc6c7ccccc7ccc6c45)nc(-c4nccc5oc6ccccc6c45)n3)ccc2c1 ; c1ccc2cc(-c3nc(-c4cc5ccccc5c5c4ccc4ccccc45)nc(-c4nccc5oc6ccccc6c45)n3)ccc2c1]

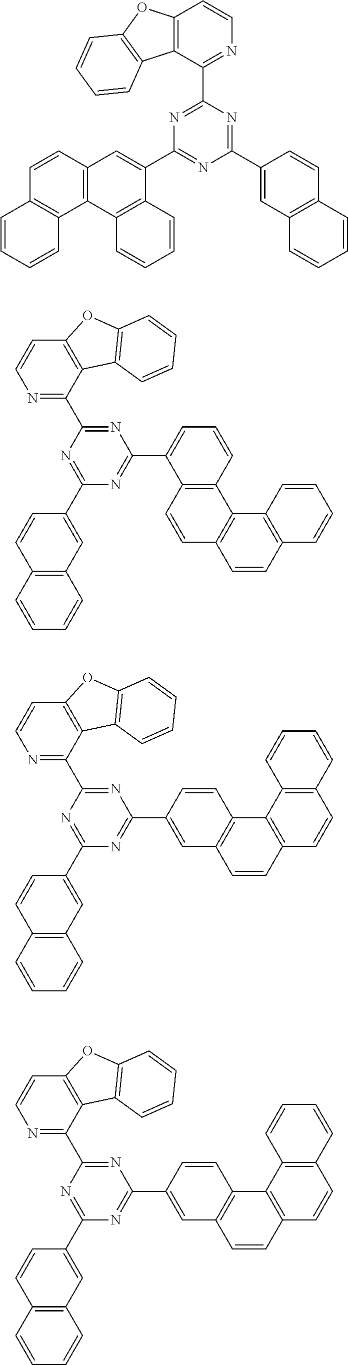 [SMILES: c1ccc2cc(-c3nc(-c4cc5ccc6ccccc6c5c5ccccc45)nc(-c4nccc5oc6ccccc6c45)n3)ccc2c1 ; c1ccc2cc(-c3nc(-c4cccc5c4ccc4ccc6ccccc6c45)nc(-c4nccc5oc6ccccc6c45)n3)ccc2c1 ; c1ccc2cc(-c3nc(-c4ccc5c(ccc6ccc7ccccc7c65)c4)nc(-c4nccc5oc6ccccc6c45)n3)ccc2c1 ; c1ccc2cc(-c3nc(-c4ccc5c(ccc6ccc7ccccc7c65)c4)nc(-c4nccc5oc6ccccc6c45)n3)ccc2c1]

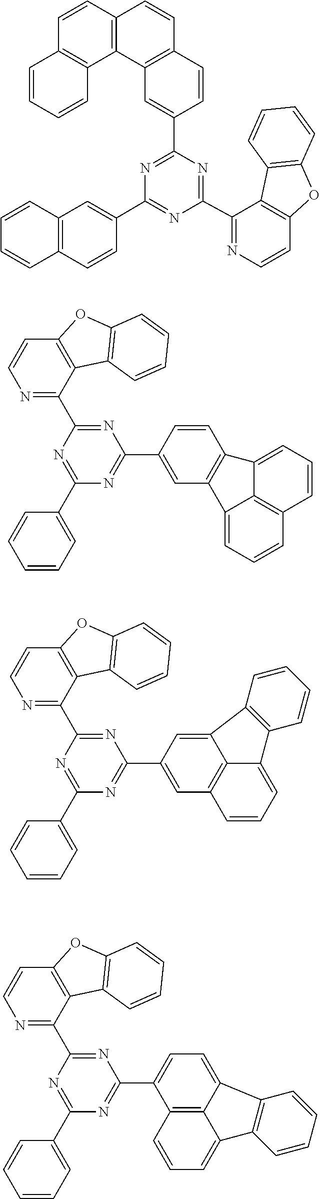

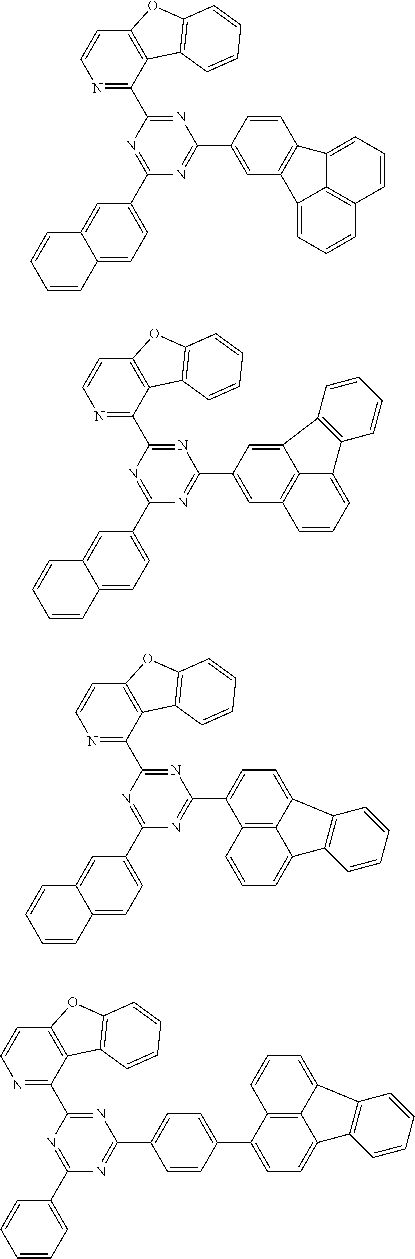 [SMILES: c1ccc2cc(-c3nc(-c4ccc5c(c4)-c4cccc6cccc-5c46)nc(-c4nccc5oc6ccccc6c45)n3)ccc2c1 ; c1ccc2c(c1)-c1cccc3cc(-c4nc(-c5ccc6ccccc6c5)nc(-c5nccc6oc7ccccc7c56)n4)cc-2c13 ; c1ccc2c(c1)-c1cccc3c(-c4nc(-c5ccc6ccccc6c5)nc(-c5nccc6oc7ccccc7c56)n4)ccc-2c13 ; c1ccc(-c2nc(-c3ccc(-c4ccc5c6c(cccc46)-c4ccccc4-5)cc3)nc(-c3nccc4oc5ccccc5c34)n2)cc1]

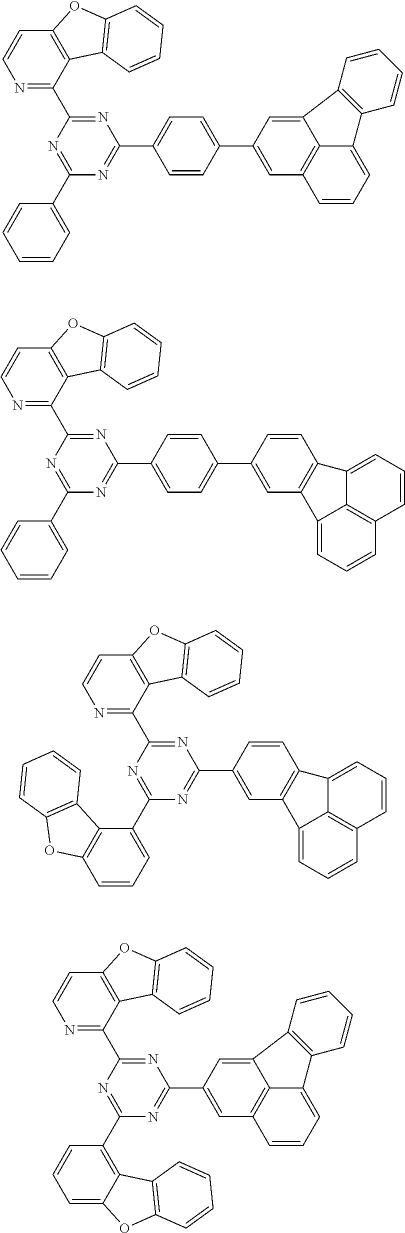 [SMILES: c1ccc(-c2nc(-c3ccc(-c4cc5c6c(cccc6c4)-c4ccccc4-5)cc3)nc(-c3nccc4oc5ccccc5c34)n2)cc1 ; c1ccc(-c2nc(-c3ccc(-c4ccc5c(c4)-c4cccc6cccc-5c46)cc3)nc(-c3nccc4oc5ccccc5c34)n2)cc1 ; c1cc2c3c(cccc3c1)-c1cc(-c3nc(-c4cccc5oc6ccccc6c45)nc(-c4nccc5oc6ccccc6c45)n3)ccc1-2 ; c1ccc2c(c1)-c1cccc3cc(-c4nc(-c5cccc6oc7ccccc7c56)nc(-c5nccc6oc7ccccc7c56)n4)cc-2c13]

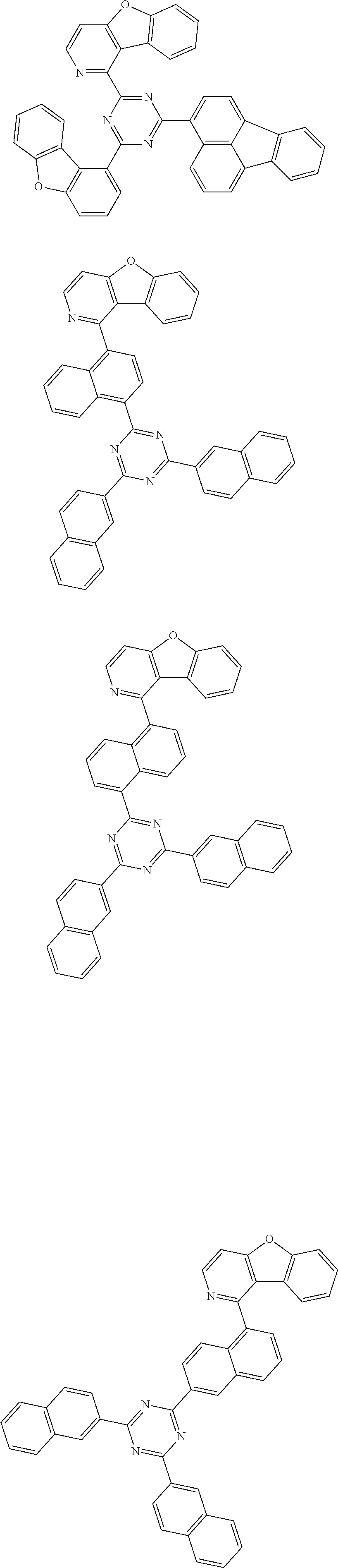 [SMILES: c1ccc2c(c1)-c1cccc3c(-c4nc(-c5cccc6oc7ccccc7c56)nc(-c5nccc6oc7ccccc7c56)n4)ccc-2c13 ; c1ccc2cc(-c3nc(-c4ccc5ccccc5c4)nc(-c4ccc(-c5nccc6oc7ccccc7c56)c5ccccc45)n3)ccc2c1 ; c1ccc2cc(-c3nc(-c4ccc5ccccc5c4)nc(-c4cccc5c(-c6nccc7oc8ccccc8c67)cccc45)n3)ccc2c1 ; c1ccc2cc(-c3nc(-c4ccc5ccccc5c4)nc(-c4ccc5c(-c6nccc7oc8ccccc8c67)cccc5c4)n3)ccc2c1]

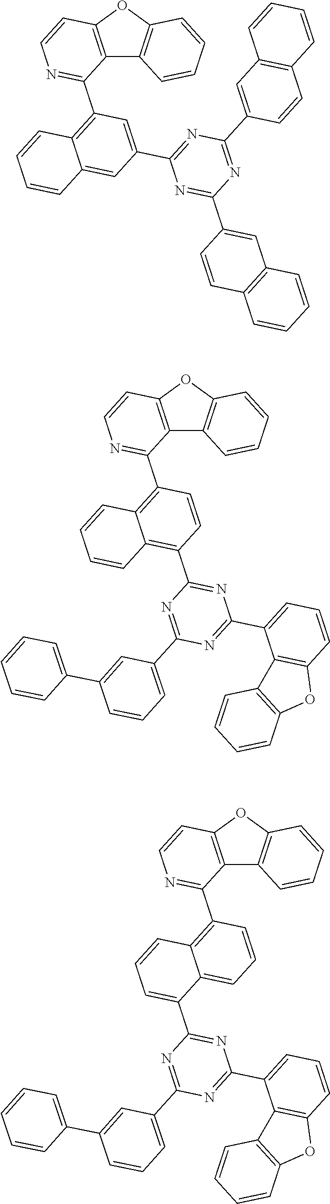 [SMILES: c1ccc2cc(-c3nc(-c4ccc5ccccc5c4)nc(-c4cc(-c5nccc6oc7ccccc7c56)c5ccccc5c4)n3)ccc2c1 ; c1ccc(-c2cccc(-c3nc(-c4ccc(-c5nccc6oc7ccccc7c56)c5ccccc45)nc(-c4cccc5oc6ccccc6c45)n3)c2)cc1 ; c1ccc(-c2cccc(-c3nc(-c4cccc5c(-c6nccc7oc8ccccc8c67)cccc45)nc(-c4cccc5oc6ccccc6c45)n3)c2)cc1]

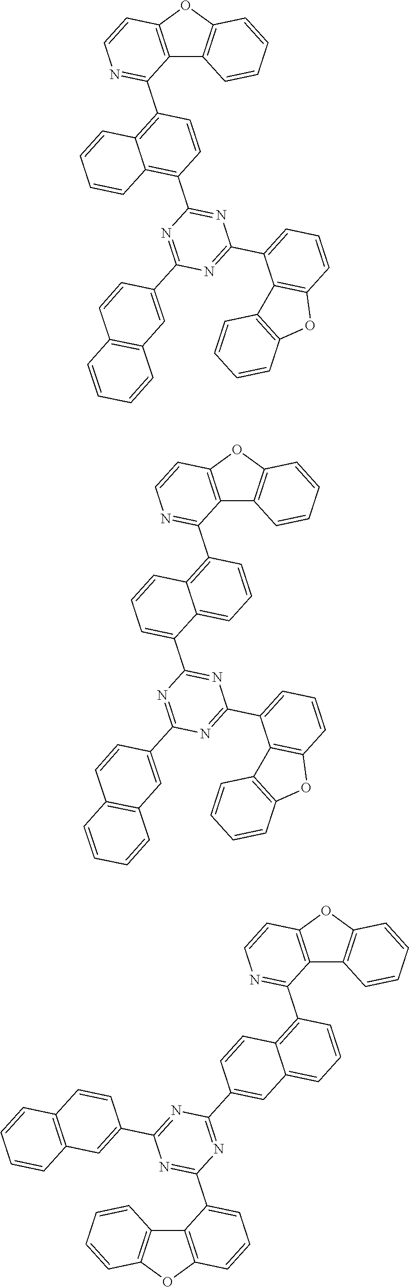 [SMILES: c1ccc2cc(-c3nc(-c4ccc(-c5nccc6oc7ccccc7c56)c5ccccc45)nc(-c4cccc5oc6ccccc6c45)n3)ccc2c1 ; c1ccc2cc(-c3nc(-c4cccc5c(-c6nccc7oc8ccccc8c67)cccc45)nc(-c4cccc5oc6ccccc6c45)n3)ccc2c1 ; c1ccc2cc(-c3nc(-c4ccc5c(-c6nccc7oc8ccccc8c67)cccc5c4)nc(-c4cccc5oc6ccccc6c45)n3)ccc2c1]

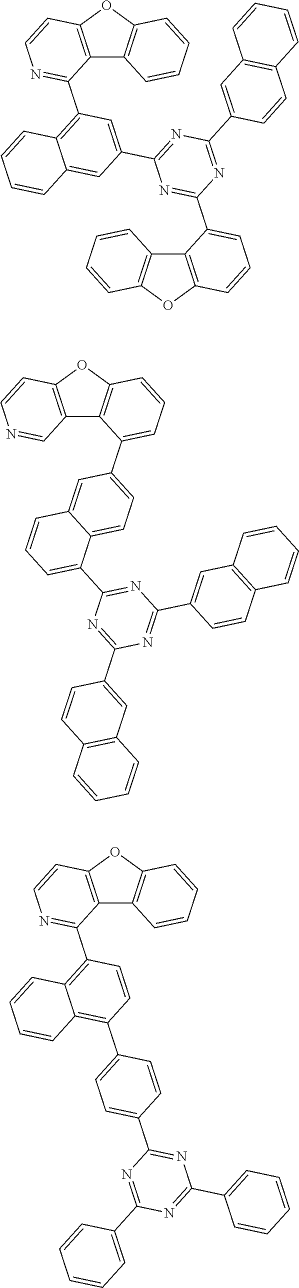 [SMILES: c1ccc2cc(-c3nc(-c4cc(-c5nccc6oc7ccccc7c56)c5ccccc5c4)nc(-c4cccc5oc6ccccc6c45)n3)ccc2c1 ; c1ccc2cc(-c3nc(-c4ccc5ccccc5c4)nc(-c4cccc5cc(-c6cccc7oc8ccncc8c67)ccc45)n3)ccc2c1 ; c1ccc(-c2nc(-c3ccccc3)nc(-c3ccc(-c4ccc(-c5nccc6oc7ccccc7c56)c5ccccc45)cc3)n2)cc1]

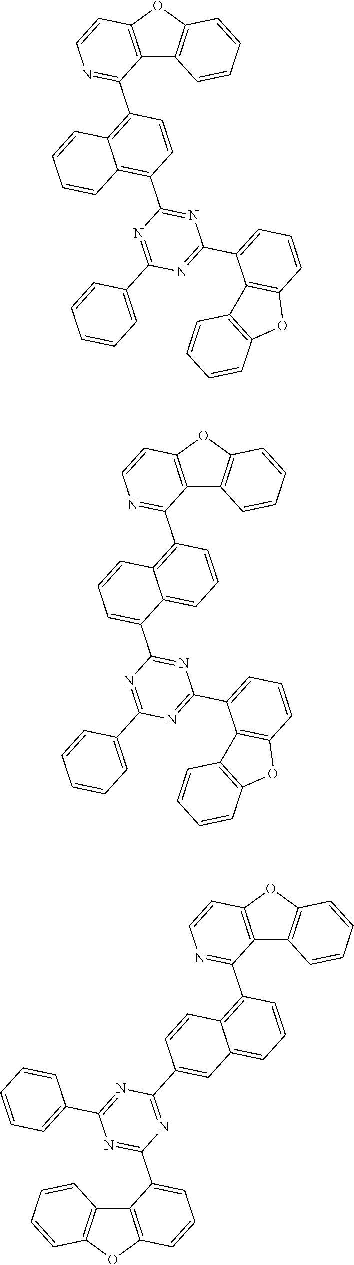 [SMILES: c1ccc(-c2nc(-c3ccc(-c4nccc5oc6ccccc6c45)c4ccccc34)nc(-c3cccc4oc5ccccc5c34)n2)cc1 ; c1ccc(-c2nc(-c3cccc4c(-c5nccc6oc7ccccc7c56)cccc34)nc(-c3cccc4oc5ccccc5c34)n2)cc1 ; c1ccc(-c2nc(-c3ccc4c(-c5nccc6oc7ccccc7c56)cccc4c3)nc(-c3cccc4oc5ccccc5c34)n2)cc1]

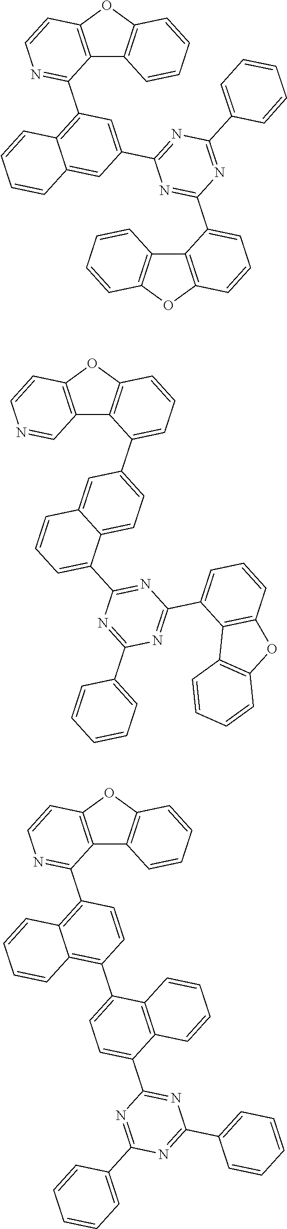 [SMILES: c1ccc(-c2nc(-c3cc(-c4nccc5oc6ccccc6c45)c4ccccc4c3)nc(-c3cccc4oc5ccccc5c34)n2)cc1 ; c1ccc(-c2nc(-c3cccc4cc(-c5cccc6oc7ccncc7c56)ccc34)nc(-c3cccc4oc5ccccc5c34)n2)cc1 ; c1ccc(-c2nc(-c3ccccc3)nc(-c3ccc(-c4ccc(-c5nccc6oc7ccccc7c56)c5ccccc45)c4ccccc34)n2)cc1]

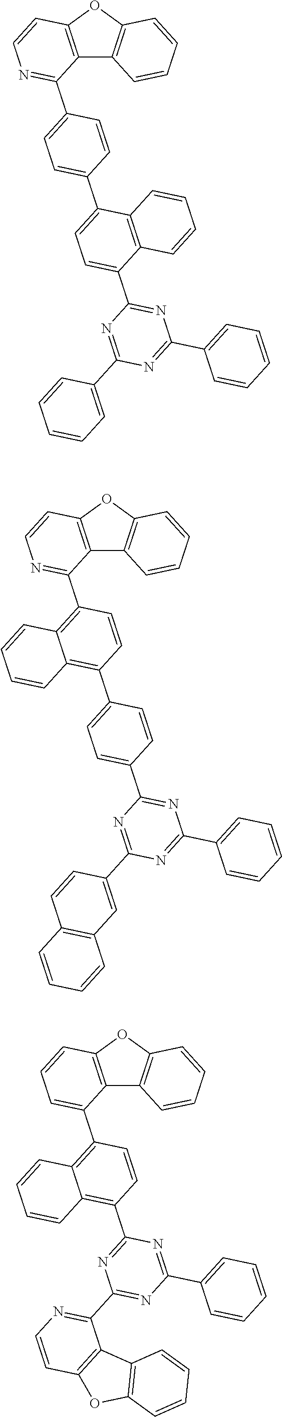 [SMILES: c1ccc(-c2nc(-c3ccccc3)nc(-c3ccc(-c4ccc(-c5nccc6oc7ccccc7c56)cc4)c4ccccc34)n2)cc1 ; c1ccc(-c2nc(-c3ccc(-c4ccc(-c5nccc6oc7ccccc7c56)c5ccccc45)cc3)nc(-c3ccc4ccccc4c3)n2)cc1 ; c1ccc(-c2nc(-c3ccc(-c4cccc5oc6ccccc6c45)c4ccccc34)nc(-c3nccc4oc5ccccc5c34)n2)cc1]

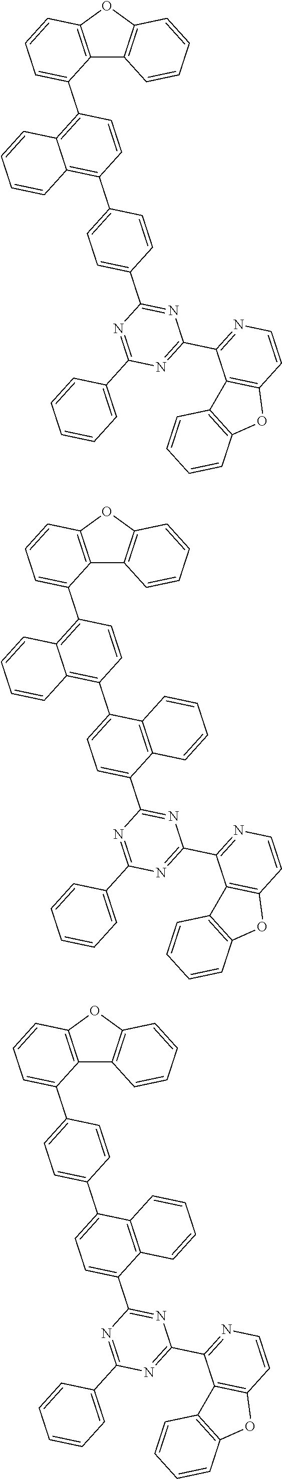 [SMILES: c1ccc(-c2nc(-c3ccc(-c4ccc(-c5cccc6oc7ccccc7c56)c5ccccc45)cc3)nc(-c3nccc4oc5ccccc5c34)n2)cc1 ; c1ccc(-c2nc(-c3ccc(-c4ccc(-c5cccc6oc7ccccc7c56)c5ccccc45)c4ccccc34)nc(-c3nccc4oc5ccccc5c34)n2)cc1 ; c1ccc(-c2nc(-c3ccc(-c4ccc(-c5cccc6oc7ccccc7c56)cc4)c4ccccc34)nc(-c3nccc4oc5ccccc5c34)n2)cc1]

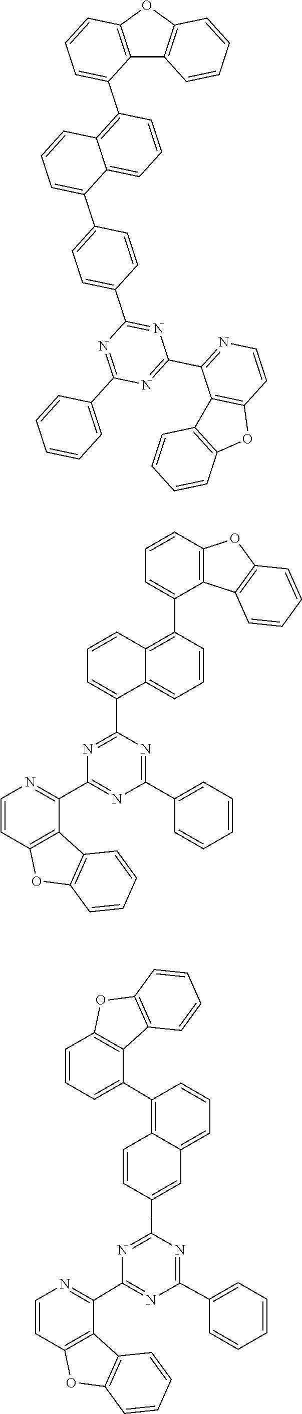

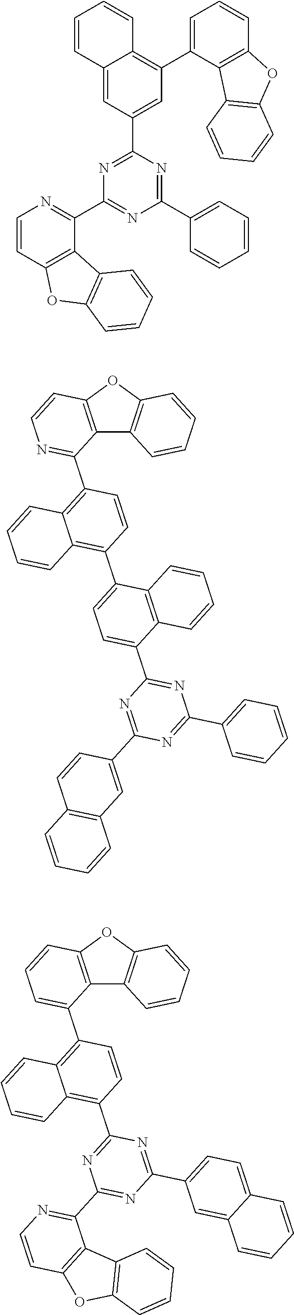 [SMILES: c1ccc(-c2nc(-c3cc(-c4cccc5oc6ccccc6c45)c4ccccc4c3)nc(-c3nccc4oc5ccccc5c34)n2)cc1 ; c1ccc(-c2nc(-c3ccc4ccccc4c3)nc(-c3ccc(-c4ccc(-c5nccc6oc7ccccc7c56)c5ccccc45)c4ccccc34)n2)cc1 ; c1ccc2cc(-c3nc(-c4ccc(-c5cccc6oc7ccccc7c56)c5ccccc45)nc(-c4nccc5oc6ccccc6c45)n3)ccc2c1]

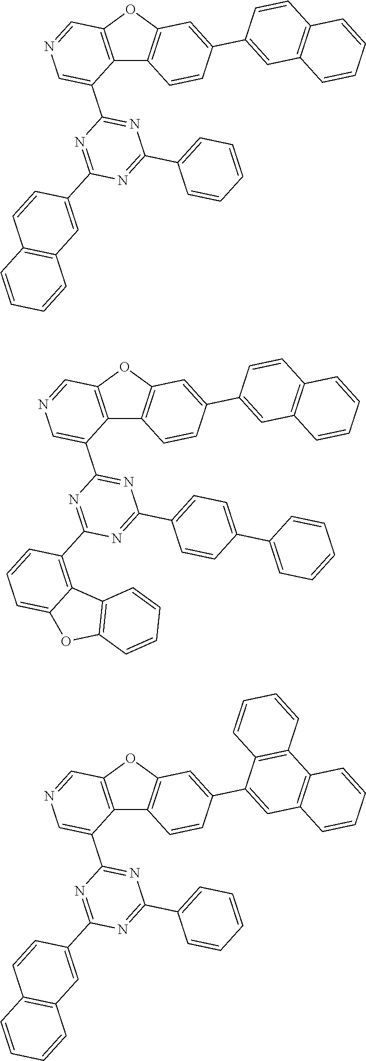 [SMILES: c1ccc(-c2nc(-c3ccc4ccccc4c3)nc(-c3cncc4oc5cc(-c6ccc7ccccc7c6)ccc5c34)n2)cc1 ; c1ccc(-c2ccc(-c3nc(-c4cccc5oc6ccccc6c45)nc(-c4cncc5oc6cc(-c7ccc8ccccc8c7)ccc6c45)n3)cc2)cc1 ; c1ccc(-c2nc(-c3ccc4ccccc4c3)nc(-c3cncc4oc5cc(-c6cc7ccccc7c7ccccc67)ccc5c34)n2)cc1]

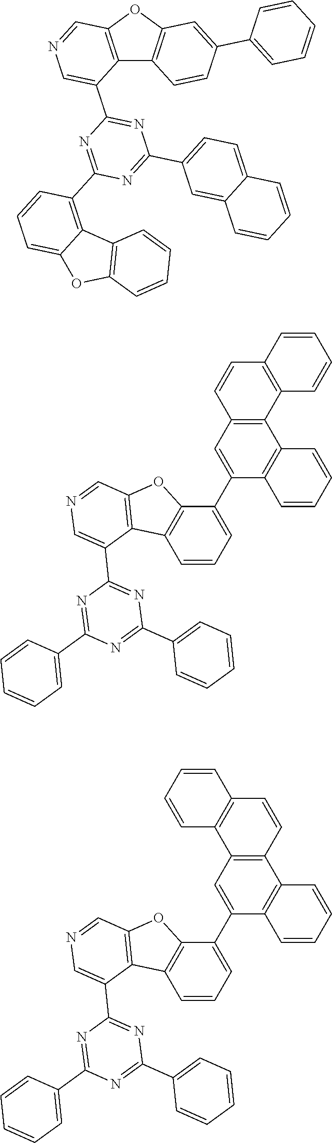 [SMILES: c1ccc(-c2ccc3c(c2)oc2cncc(-c4nc(-c5ccc6ccccc6c5)nc(-c5cccc6oc7ccccc7c56)n4)c23)cc1 ; c1ccc(-c2nc(-c3ccccc3)nc(-c3cncc4oc5c(-c6cc7ccc8ccccc8c7c7ccccc67)cccc5c34)n2)cc1 ; c1ccc(-c2nc(-c3ccccc3)nc(-c3cncc4oc5c(-c6cc7c8ccccc8ccc7c7ccccc67)cccc5c34)n2)cc1]

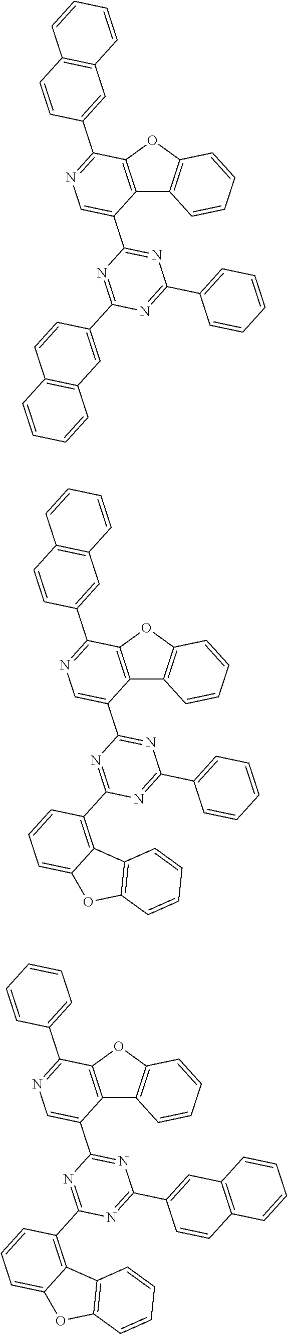 [SMILES: c1ccc(-c2nc(-c3ccc4ccccc4c3)nc(-c3cnc(-c4ccc5ccccc5c4)c4oc5ccccc5c34)n2)cc1 ; c1ccc(-c2nc(-c3cccc4oc5ccccc5c34)nc(-c3cnc(-c4ccc5ccccc5c4)c4oc5ccccc5c34)n2)cc1 ; c1ccc(-c2ncc(-c3nc(-c4ccc5ccccc5c4)nc(-c4cccc5oc6ccccc6c45)n3)c3c2oc2ccccc23)cc1]

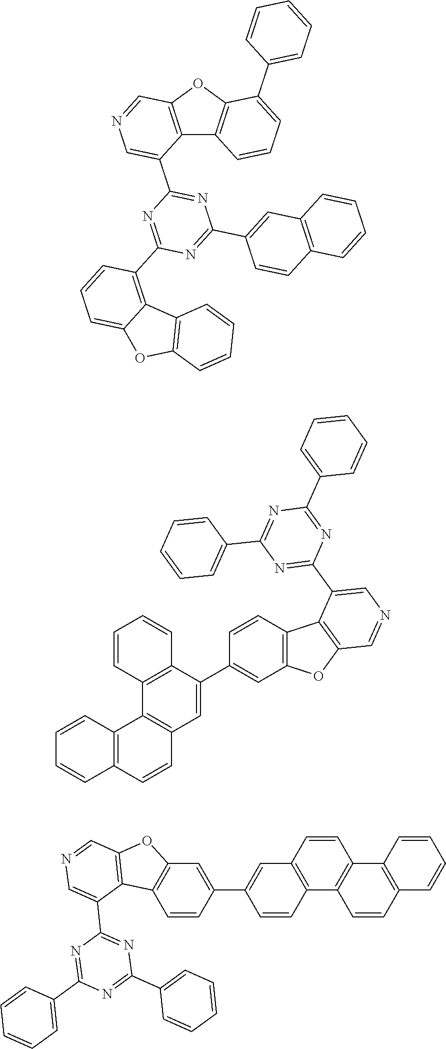 [SMILES: c1ccc(-c2cccc3c2oc2cncc(-c4nc(-c5ccc6ccccc6c5)nc(-c5cccc6oc7ccccc7c56)n4)c23)cc1 ; c1ccc(-c2nc(-c3ccccc3)nc(-c3cncc4oc5cc(-c6cc7ccc8ccccc8c7c7ccccc67)ccc5c34)n2)cc1 ; c1ccc(-c2nc(-c3ccccc3)nc(-c3cncc4oc5cc(-c6ccc7c(ccc8c9ccccc9ccc78)c6)ccc5c34)n2)cc1]

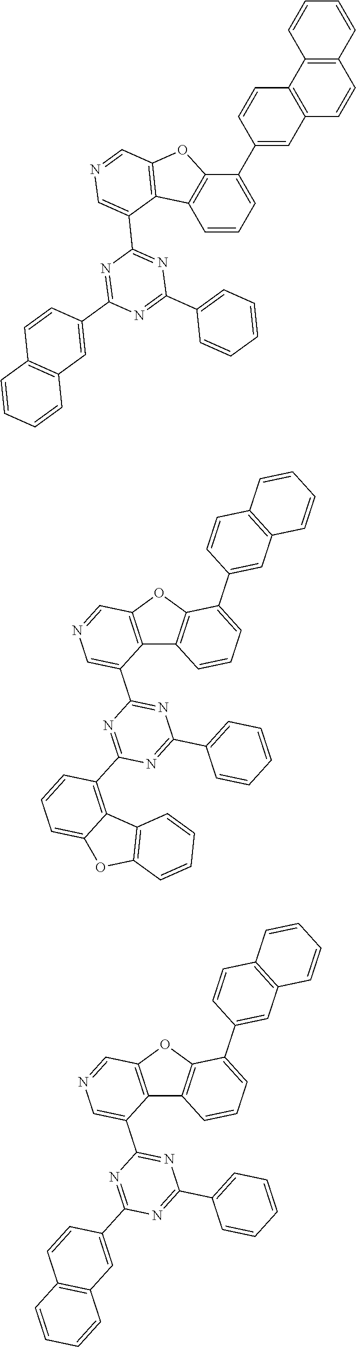 [SMILES: c1ccc(-c2nc(-c3ccc4ccccc4c3)nc(-c3cncc4oc5c(-c6ccc7c(ccc8ccccc87)c6)cccc5c34)n2)cc1 ; c1ccc(-c2nc(-c3cccc4oc5ccccc5c34)nc(-c3cncc4oc5c(-c6ccc7ccccc7c6)cccc5c34)n2)cc1 ; c1ccc(-c2nc(-c3ccc4ccccc4c3)nc(-c3cncc4oc5c(-c6ccc7ccccc7c6)cccc5c34)n2)cc1]

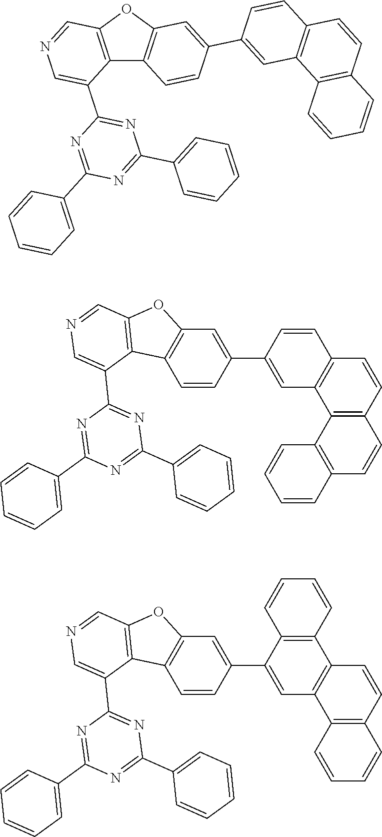 [SMILES: c1ccc(-c2nc(-c3ccccc3)nc(-c3cncc4oc5cc(-c6ccc7ccc8ccccc8c7c6)ccc5c34)n2)cc1 ; c1ccc(-c2nc(-c3ccccc3)nc(-c3cncc4oc5cc(-c6ccc7ccc8ccc9ccccc9c8c7c6)ccc5c34)n2)cc1 ; c1ccc(-c2nc(-c3ccccc3)nc(-c3cncc4oc5cc(-c6cc7c8ccccc8ccc7c7ccccc67)ccc5c34)n2)cc1]

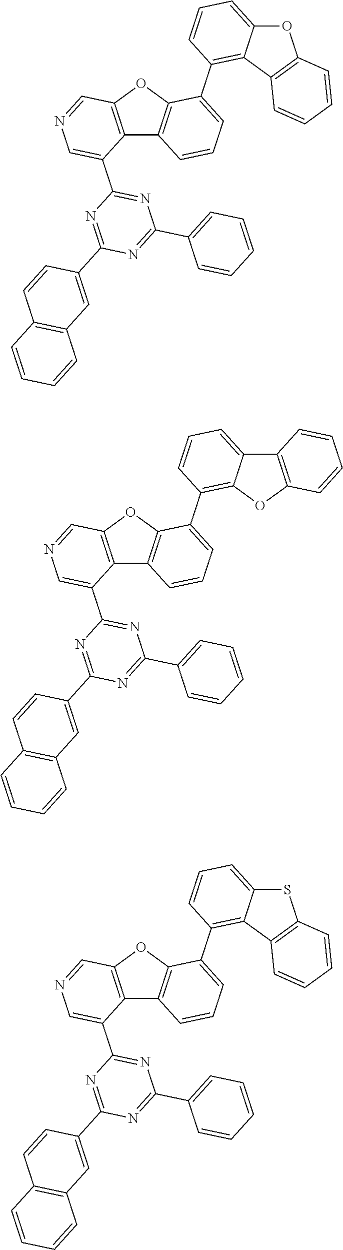 [SMILES: c1ccc(-c2nc(-c3ccc4ccccc4c3)nc(-c3cncc4oc5c(-c6cccc7oc8ccccc8c67)cccc5c34)n2)cc1 ; c1ccc(-c2nc(-c3ccc4ccccc4c3)nc(-c3cncc4oc5c(-c6cccc7c6oc6ccccc67)cccc5c34)n2)cc1 ; c1ccc(-c2nc(-c3ccc4ccccc4c3)nc(-c3cncc4oc5c(-c6cccc7sc8ccccc8c67)cccc5c34)n2)cc1]

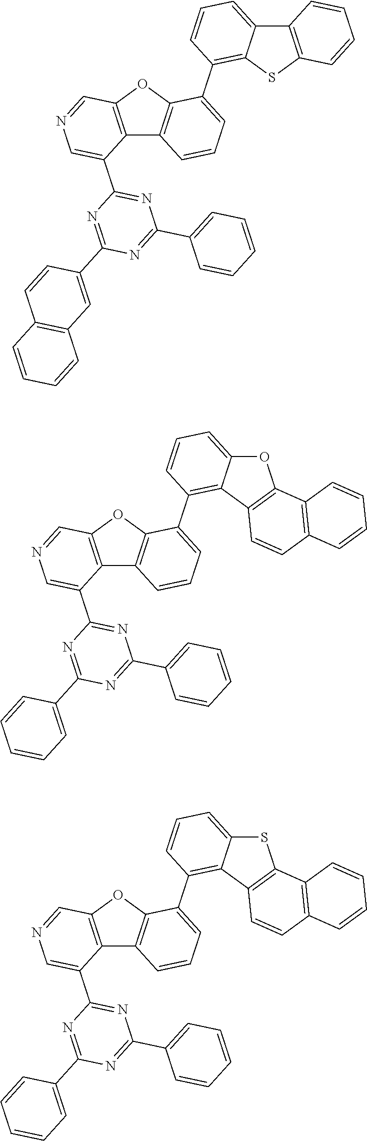 [SMILES: c1ccc(-c2nc(-c3ccc4ccccc4c3)nc(-c3cncc4oc5c(-c6cccc7c6sc6ccccc67)cccc5c34)n2)cc1 ; c1ccc(-c2nc(-c3ccccc3)nc(-c3cncc4oc5c(-c6cccc7oc8c9ccccc9ccc8c67)cccc5c34)n2)cc1 ; c1ccc(-c2nc(-c3ccccc3)nc(-c3cncc4oc5c(-c6cccc7sc8c9ccccc9ccc8c67)cccc5c34)n2)cc1]

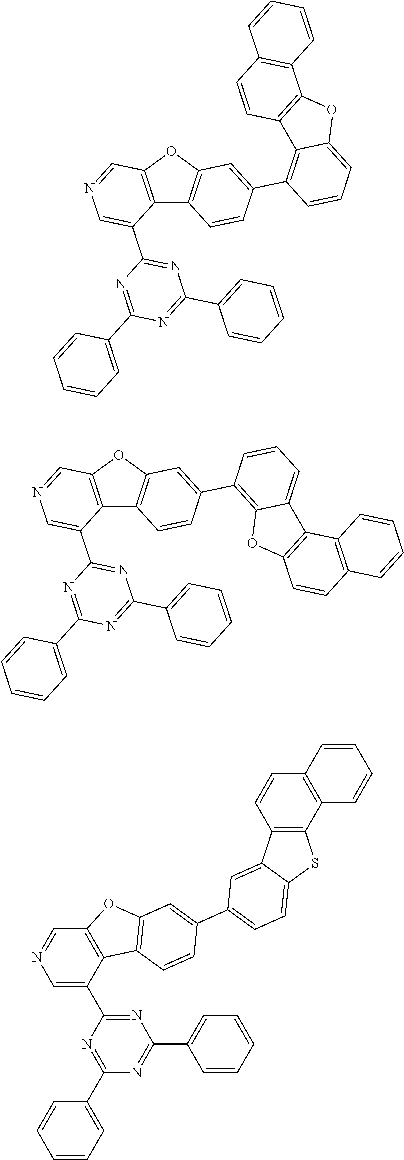 [SMILES: c1ccc(-c2nc(-c3ccccc3)nc(-c3cncc4oc5cc(-c6cccc7oc8c9ccccc9ccc8c67)ccc5c34)n2)cc1 ; c1ccc(-c2nc(-c3ccccc3)nc(-c3cncc4oc5cc(-c6cccc7c6oc6ccc8ccccc8c67)ccc5c34)n2)cc1 ; c1ccc(-c2nc(-c3ccccc3)nc(-c3cncc4oc5cc(-c6ccc7sc8c9ccccc9ccc8c7c6)ccc5c34)n2)cc1]

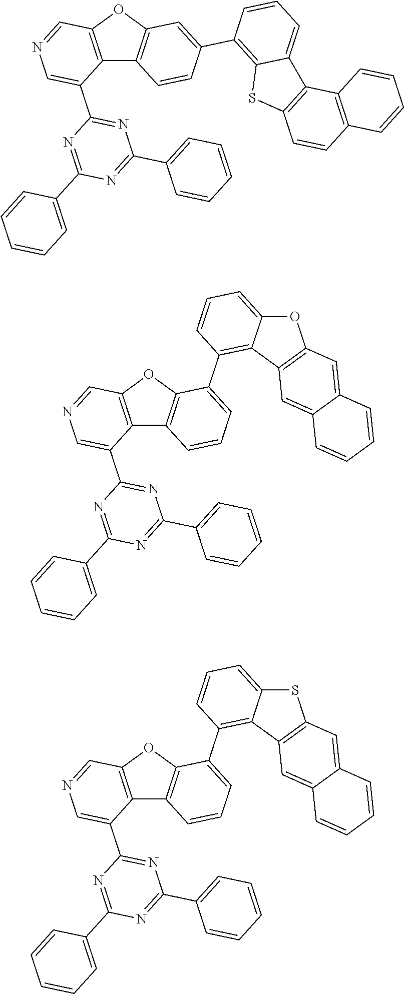 [SMILES: c1ccc(-c2nc(-c3ccccc3)nc(-c3cncc4oc5cc(-c6cccc7c6sc6ccc8ccccc8c67)ccc5c34)n2)cc1 ; c1ccc(-c2nc(-c3ccccc3)nc(-c3cncc4oc5c(-c6cccc7oc8cc9ccccc9cc8c67)cccc5c34)n2)cc1 ; c1ccc(-c2nc(-c3ccccc3)nc(-c3cncc4oc5c(-c6cccc7sc8cc9ccccc9cc8c67)cccc5c34)n2)cc1]

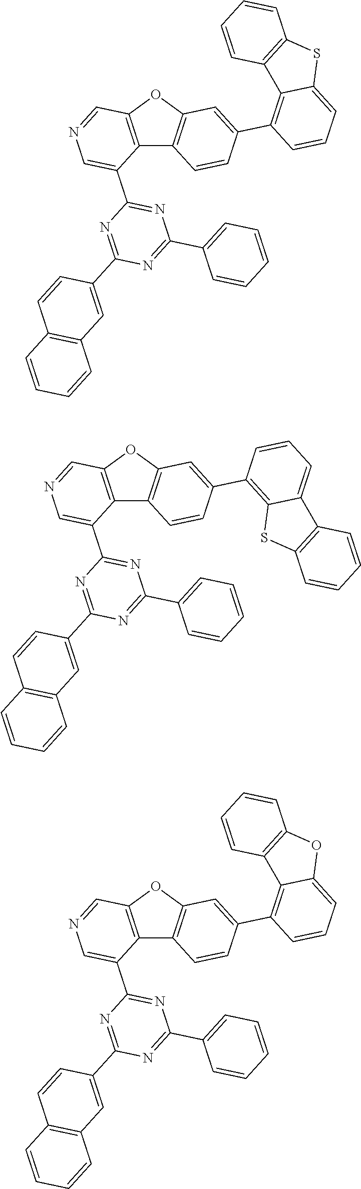 [SMILES: c1ccc(-c2nc(-c3ccc4ccccc4c3)nc(-c3cncc4oc5cc(-c6cccc7sc8ccccc8c67)ccc5c34)n2)cc1 ; c1ccc(-c2nc(-c3ccc4ccccc4c3)nc(-c3cncc4oc5cc(-c6cccc7c6sc6ccccc67)ccc5c34)n2)cc1 ; c1ccc(-c2nc(-c3ccc4ccccc4c3)nc(-c3cncc4oc5cc(-c6cccc7oc8ccccc8c67)ccc5c34)n2)cc1]

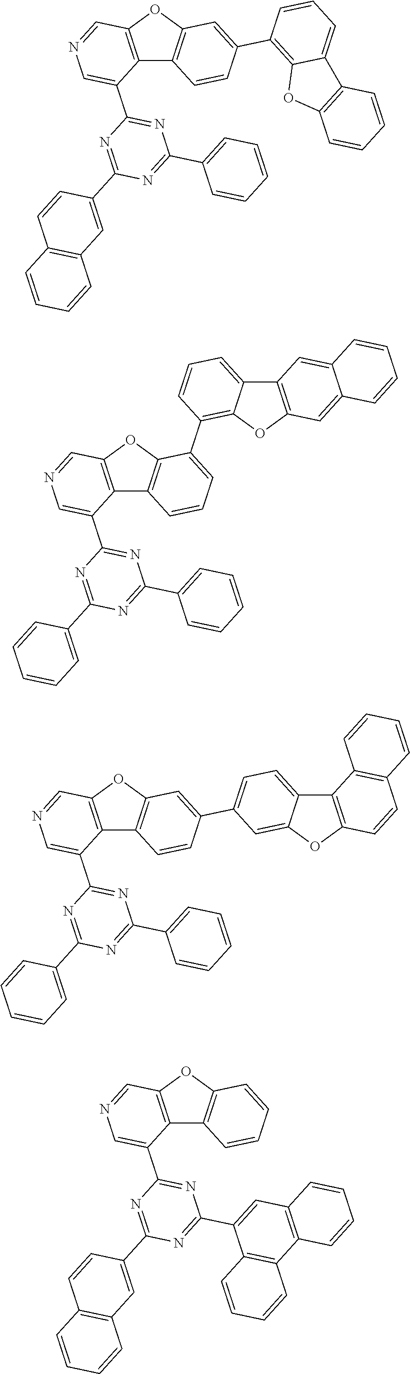 [SMILES: c1ccc(-c2nc(-c3ccc4ccccc4c3)nc(-c3cncc4oc5cc(-c6cccc7c6oc6ccccc67)ccc5c34)n2)cc1 ; c1ccc(-c2nc(-c3ccccc3)nc(-c3cncc4oc5c(-c6cccc7c6oc6cc8ccccc8cc67)cccc5c34)n2)cc1 ; c1ccc(-c2nc(-c3ccccc3)nc(-c3cncc4oc5cc(-c6ccc7c(c6)oc6ccc8ccccc8c67)ccc5c34)n2)cc1 ; c1ccc2cc(-c3nc(-c4cc5ccccc5c5ccccc45)nc(-c4cncc5oc6ccccc6c45)n3)ccc2c1]

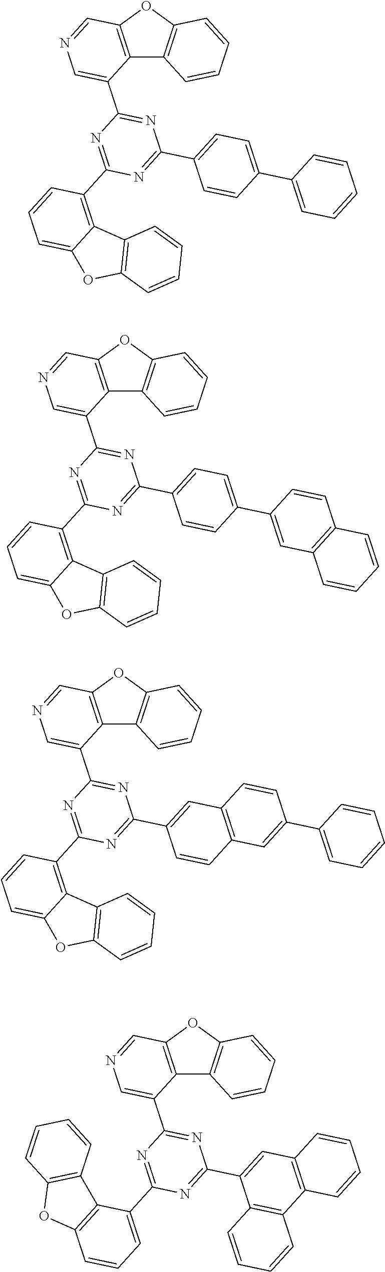

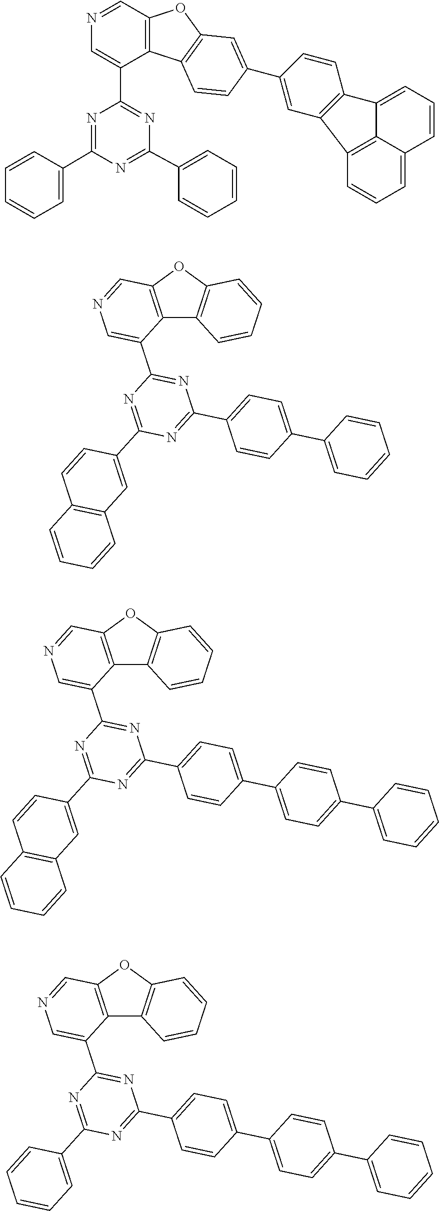 [SMILES: c1ccc(-c2nc(-c3ccccc3)nc(-c3cncc4oc5cc(-c6ccc7c(c6)-c6cccc8cccc-7c68)ccc5c34)n2)cc1 ; c1ccc(-c2ccc(-c3nc(-c4ccc5ccccc5c4)nc(-c4cncc5oc6ccccc6c45)n3)cc2)cc1 ; c1ccc(-c2ccc(-c3ccc(-c4nc(-c5ccc6ccccc6c5)nc(-c5cncc6oc7ccccc7c56)n4)cc3)cc2)cc1 ; c1ccc(-c2ccc(-c3ccc(-c4nc(-c5ccccc5)nc(-c5cncc6oc7ccccc7c56)n4)cc3)cc2)cc1]

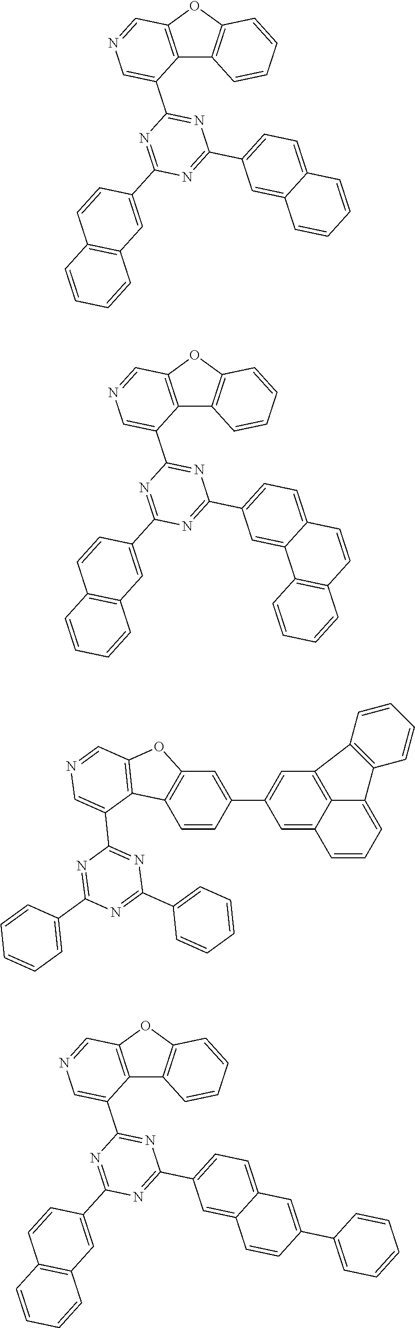 [SMILES: c1ccc2cc(-c3nc(-c4ccc5ccccc5c4)nc(-c4cncc5oc6ccccc6c45)n3)ccc2c1 ; c1ccc2cc(-c3nc(-c4ccc5ccc6ccccc6c5c4)nc(-c4cncc5oc6ccccc6c45)n3)ccc2c1 ; c1ccc(-c2nc(-c3ccccc3)nc(-c3cncc4oc5cc(-c6cc7c8c(cccc8c6)-c6ccccc6-7)ccc5c34)n2)cc1 ; c1ccc(-c2ccc3cc(-c4nc(-c5ccc6ccccc6c5)nc(-c5cncc6oc7ccccc7c56)n4)ccc3c2)cc1]

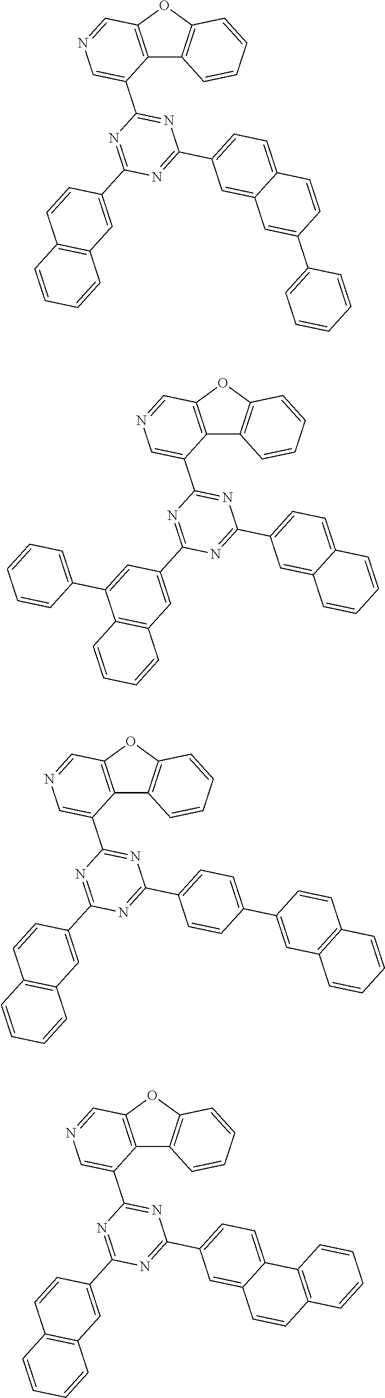 [SMILES: c1ccc(-c2ccc3ccc(-c4nc(-c5ccc6ccccc6c5)nc(-c5cncc6oc7ccccc7c56)n4)cc3c2)cc1 ; c1ccc(-c2cc(-c3nc(-c4ccc5ccccc5c4)nc(-c4cncc5oc6ccccc6c45)n3)cc3ccccc23)cc1 ; c1ccc2cc(-c3ccc(-c4nc(-c5ccc6ccccc6c5)nc(-c5cncc6oc7ccccc7c56)n4)cc3)ccc2c1 ; c1ccc2cc(-c3nc(-c4ccc5c(ccc6ccccc65)c4)nc(-c4cncc5oc6ccccc6c45)n3)ccc2c1]

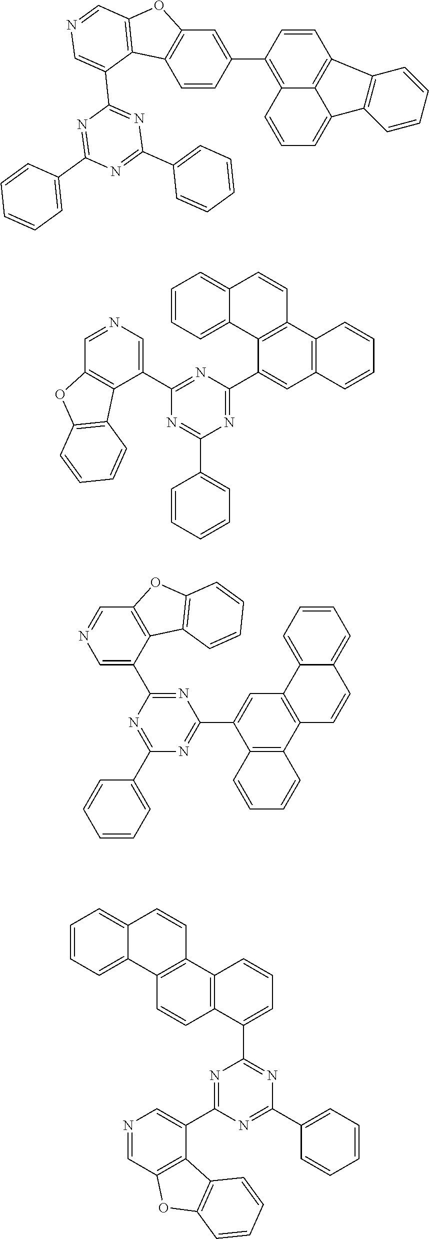

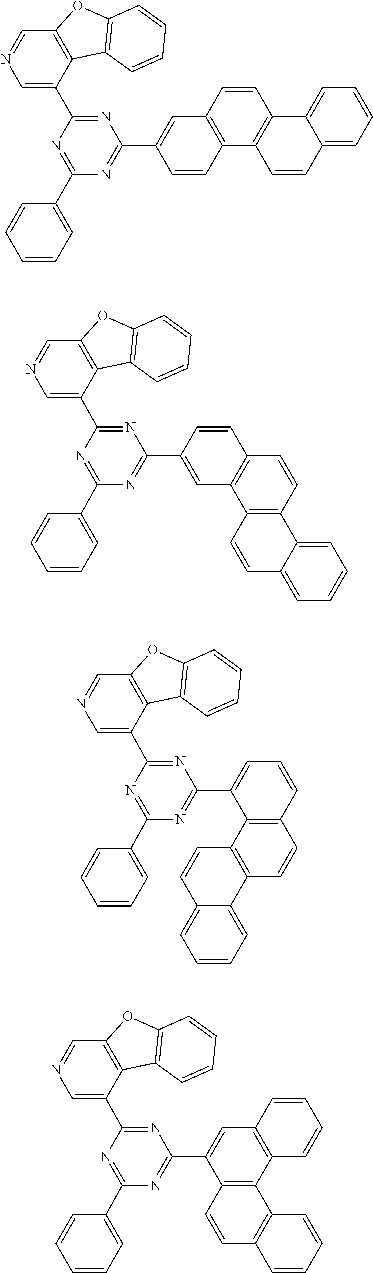 [SMILES: c1ccc(-c2nc(-c3ccc4c(ccc5c6ccccc6ccc45)c3)nc(-c3cncc4oc5ccccc5c34)n2)cc1 ; c1ccc(-c2nc(-c3ccc4ccc5c6ccccc6ccc5c4c3)nc(-c3cncc4oc5ccccc5c34)n2)cc1 ; c1ccc(-c2nc(-c3cccc4ccc5c6ccccc6ccc5c34)nc(-c3cncc4oc5ccccc5c34)n2)cc1 ; c1ccc(-c2nc(-c3cc4ccccc4c4c3ccc3ccccc34)nc(-c3cncc4oc5ccccc5c34)n2)cc1]

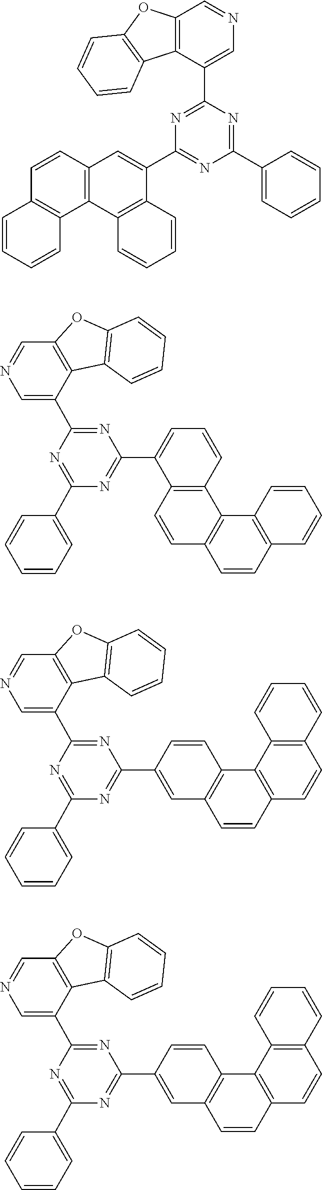 [SMILES: c1ccc(-c2nc(-c3cc4ccc5ccccc5c4c4ccccc34)nc(-c3cncc4oc5ccccc5c34)n2)cc1 ; c1ccc(-c2nc(-c3cccc4c3ccc3ccc5ccccc5c34)nc(-c3cncc4oc5ccccc5c34)n2)cc1 ; c1ccc(-c2nc(-c3ccc4c(ccc5ccc6ccccc6c54)c3)nc(-c3cncc4oc5ccccc5c34)n2)cc1 ; c1ccc(-c2nc(-c3ccc4c(ccc5ccc6ccccc6c54)c3)nc(-c3cncc4oc5ccccc5c34)n2)cc1]

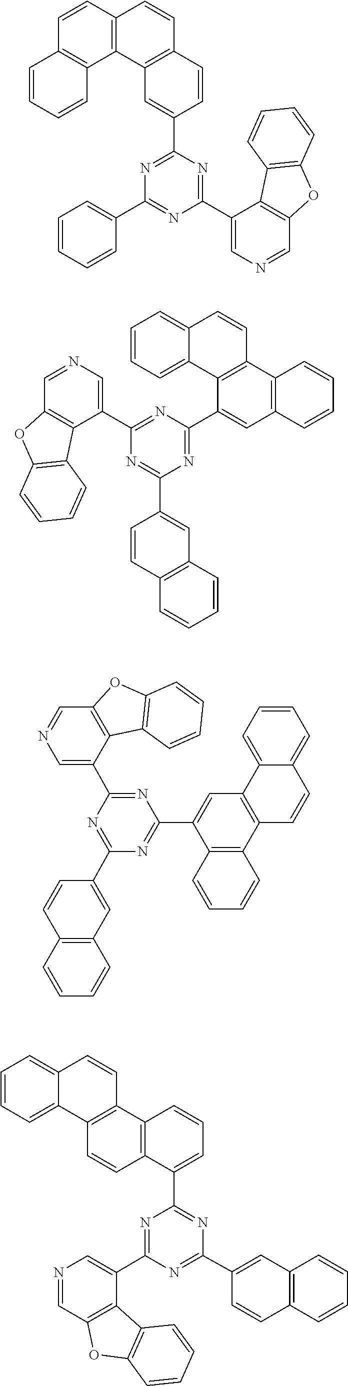 [SMILES: c1ccc(-c2nc(-c3ccc4ccc5ccc6ccccc6c5c4c3)nc(-c3cncc4oc5ccccc5c34)n2)cc1 ; c1ccc2cc(-c3nc(-c4cncc5oc6ccccc6c45)nc(-c4cc5ccccc5c5ccc6ccccc6c45)n3)ccc2c1 ; c1ccc2cc(-c3nc(-c4cc5c6ccccc6ccc5c5ccccc45)nc(-c4cncc5oc6ccccc6c45)n3)ccc2c1 ; c1ccc2cc(-c3nc(-c4cccc5c4ccc4c6ccccc6ccc54)nc(-c4cncc5oc6ccccc6c45)n3)ccc2c1]

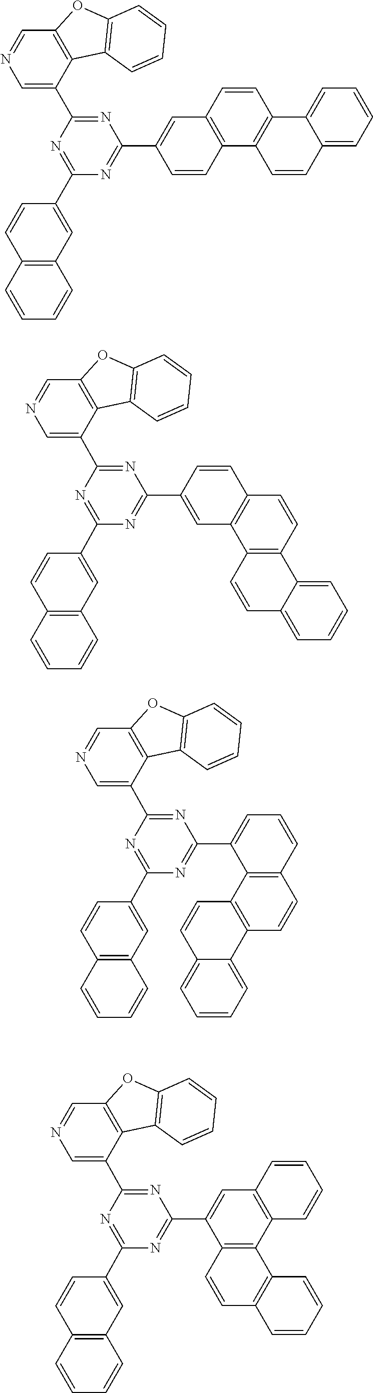 [SMILES: c1ccc2cc(-c3nc(-c4ccc5c(ccc6c7ccccc7ccc56)c4)nc(-c4cncc5oc6ccccc6c45)n3)ccc2c1 ; c1ccc2cc(-c3nc(-c4ccc5ccc6c7ccccc7ccc6c5c4)nc(-c4cncc5oc6ccccc6c45)n3)ccc2c1 ; c1ccc2cc(-c3nc(-c4cccc5ccc6c7ccccc7ccc6c45)nc(-c4cncc5oc6ccccc6c45)n3)ccc2c1 ; c1ccc2cc(-c3nc(-c4cc5ccccc5c5c4ccc4ccccc45)nc(-c4cncc5oc6ccccc6c45)n3)ccc2c1]

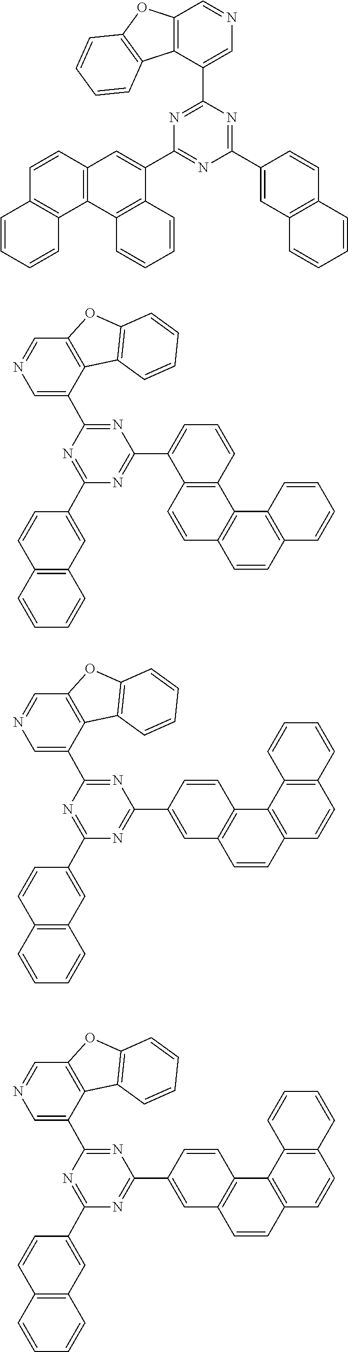

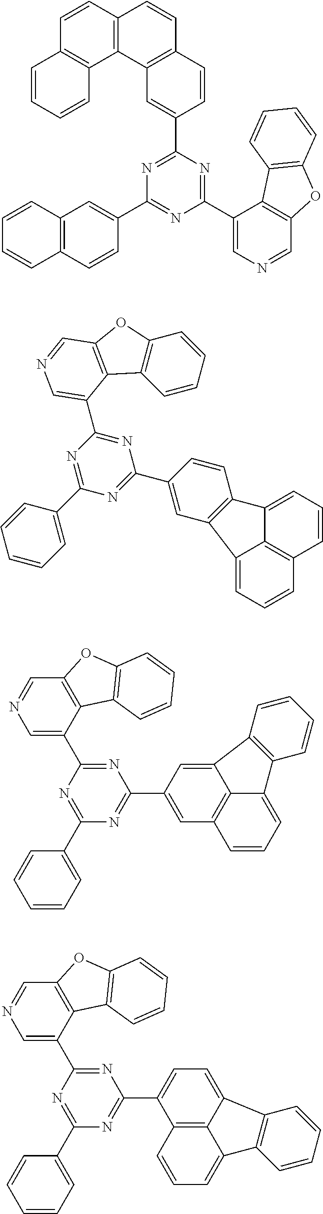 [SMILES: c1ccc2cc(-c3nc(-c4ccc5ccc6ccc7ccccc7c6c5c4)nc(-c4cncc5oc6ccccc6c45)n3)ccc2c1 ; c1ccc(-c2nc(-c3ccc4c(c3)-c3cccc5cccc-4c35)nc(-c3cncc4oc5ccccc5c34)n2)cc1 ; c1ccc(-c2nc(-c3cc4c5c(cccc5c3)-c3ccccc3-4)nc(-c3cncc4oc5ccccc5c34)n2)cc1 ; c1ccc(-c2nc(-c3ccc4c5c(cccc35)-c3ccccc3-4)nc(-c3cncc4oc5ccccc5c34)n2)cc1]

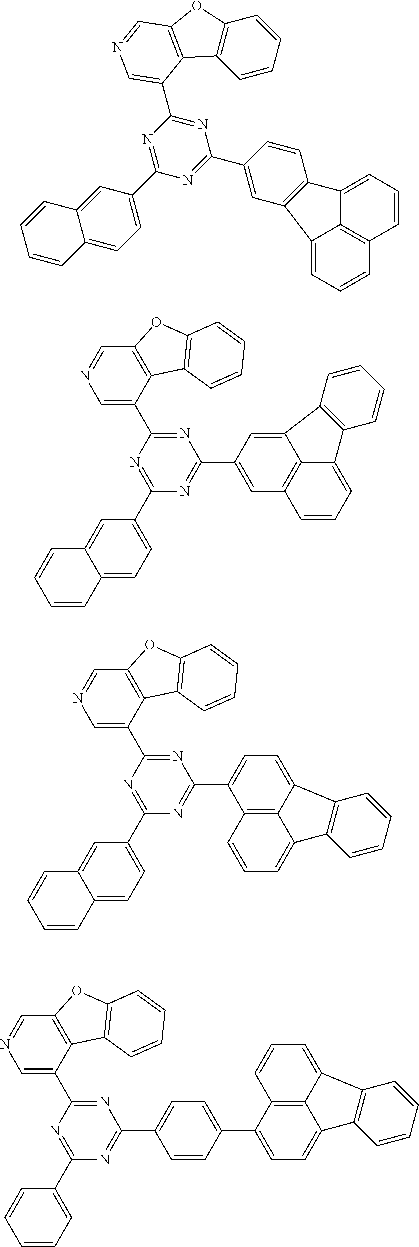 [SMILES: c1ccc2cc(-c3nc(-c4ccc5c(c4)-c4cccc6cccc-5c46)nc(-c4cncc5oc6ccccc6c45)n3)ccc2c1 ; c1ccc2c(c1)-c1cccc3cc(-c4nc(-c5ccc6ccccc6c5)nc(-c5cncc6oc7ccccc7c56)n4)cc-2c13 ; c1ccc2c(c1)-c1cccc3c(-c4nc(-c5ccc6ccccc6c5)nc(-c5cncc6oc7ccccc7c56)n4)ccc-2c13 ; c1ccc(-c2nc(-c3ccc(-c4ccc5c6c(cccc46)-c4ccccc4-5)cc3)nc(-c3cncc4oc5ccccc5c34)n2)cc1]

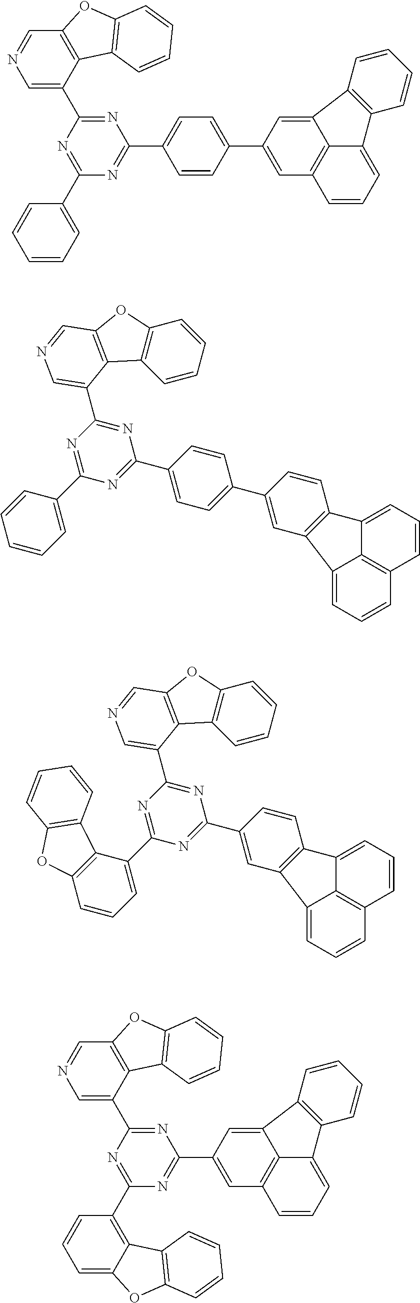 [SMILES: c1ccc(-c2nc(-c3ccc(-c4cc5c6c(cccc6c4)-c4ccccc4-5)cc3)nc(-c3cncc4oc5ccccc5c34)n2)cc1 ; c1ccc(-c2nc(-c3ccc(-c4ccc5c(c4)-c4cccc6cccc-5c46)cc3)nc(-c3cncc4oc5ccccc5c34)n2)cc1 ; c1cc2c3c(cccc3c1)-c1cc(-c3nc(-c4cccc5oc6ccccc6c45)nc(-c4cncc5oc6ccccc6c45)n3)ccc1-2 ; c1ccc2c(c1)-c1cccc3cc(-c4nc(-c5cccc6oc7ccccc7c56)nc(-c5cncc6oc7ccccc7c56)n4)cc-2c13]

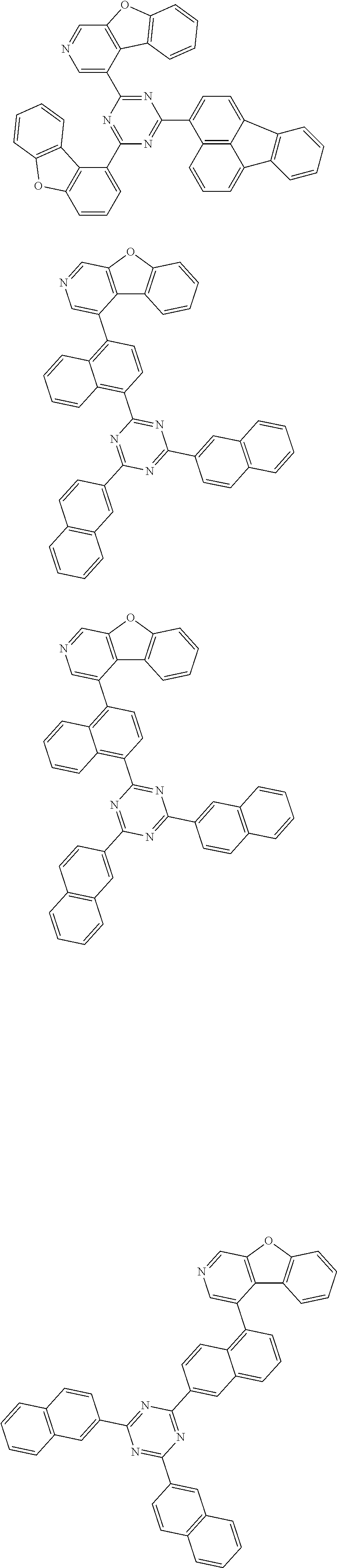 [SMILES: c1ccc2c(c1)-c1cccc3c(-c4nc(-c5cccc6oc7ccccc7c56)nc(-c5cncc6oc7ccccc7c56)n4)ccc-2c13 ; c1ccc2cc(-c3nc(-c4ccc5ccccc5c4)nc(-c4ccc(-c5cncc6oc7ccccc7c56)c5ccccc45)n3)ccc2c1 ; c1ccc2cc(-c3nc(-c4ccc5ccccc5c4)nc(-c4ccc(-c5cncc6oc7ccccc7c56)c5ccccc45)n3)ccc2c1 ; c1ccc2cc(-c3nc(-c4ccc5ccccc5c4)nc(-c4ccc5c(-c6cncc7oc8ccccc8c67)cccc5c4)n3)ccc2c1]

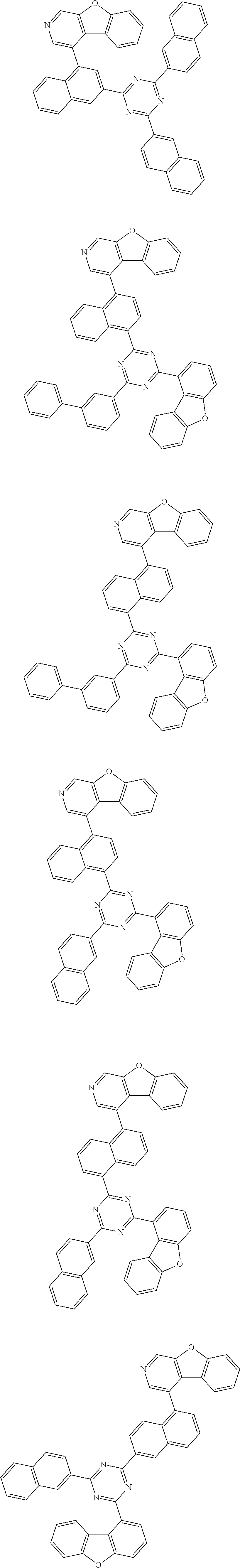

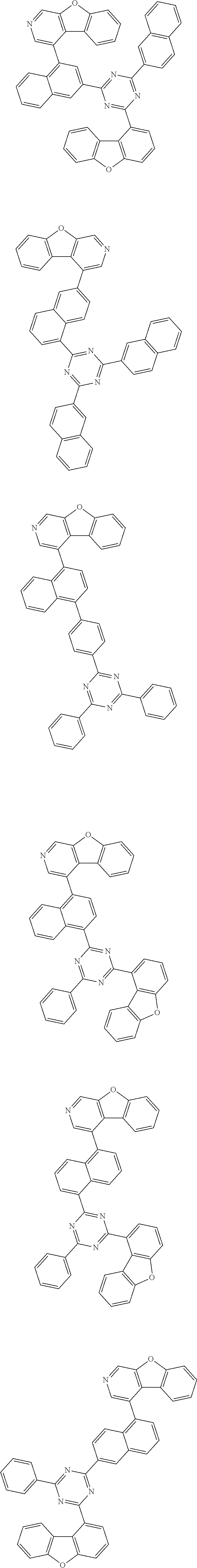

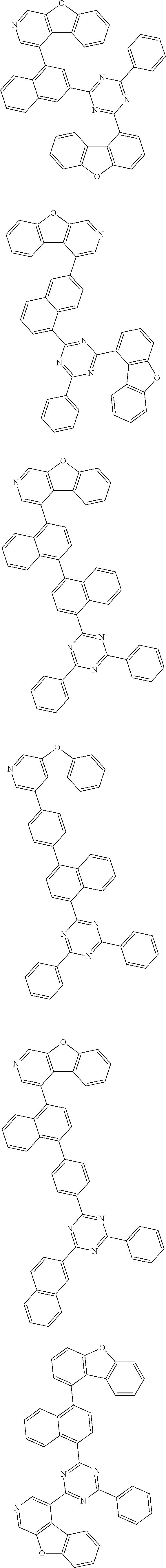

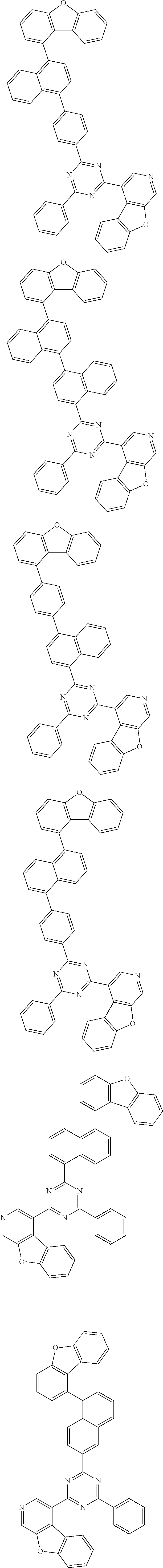

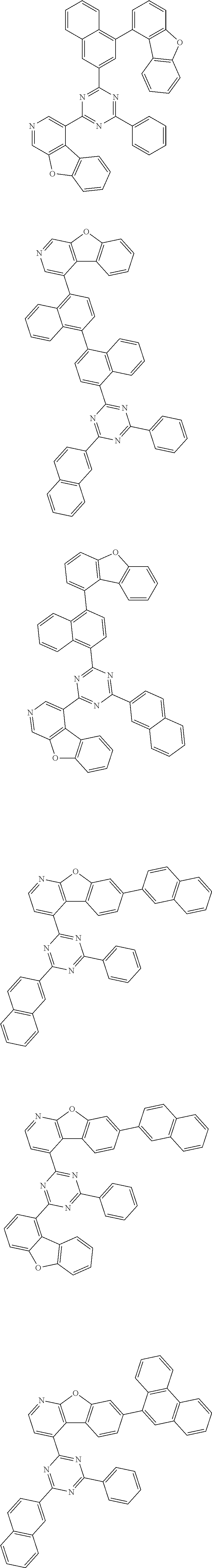

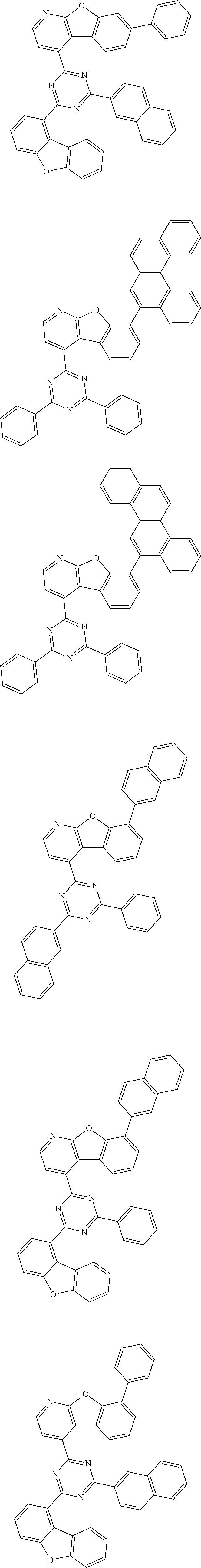 [SMILES: c1ccc(-c2ccc3c(c2)oc2nccc(-c4nc(-c5ccc6ccccc6c5)nc(-c5cccc6oc7ccccc7c56)n4)c23)cc1 ; c1ccc(-c2nc(-c3ccccc3)nc(-c3ccnc4oc5c(-c6cc7ccc8ccccc8c7c7ccccc67)cccc5c34)n2)cc1 ; c1ccc(-c2nc(-c3ccccc3)nc(-c3ccnc4oc5c(-c6cc7c8ccccc8ccc7c7ccccc67)cccc5c34)n2)cc1 ; c1ccc(-c2nc(-c3ccc4ccccc4c3)nc(-c3ccnc4oc5c(-c6ccc7ccccc7c6)cccc5c34)n2)cc1 ; c1ccc(-c2nc(-c3cccc4oc5ccccc5c34)nc(-c3ccnc4oc5c(-c6ccc7ccccc7c6)cccc5c34)n2)cc1 ; c1ccc(-c2cccc3c2oc2nccc(-c4nc(-c5ccc6ccccc6c5)nc(-c5cccc6oc7ccccc7c56)n4)c23)cc1]

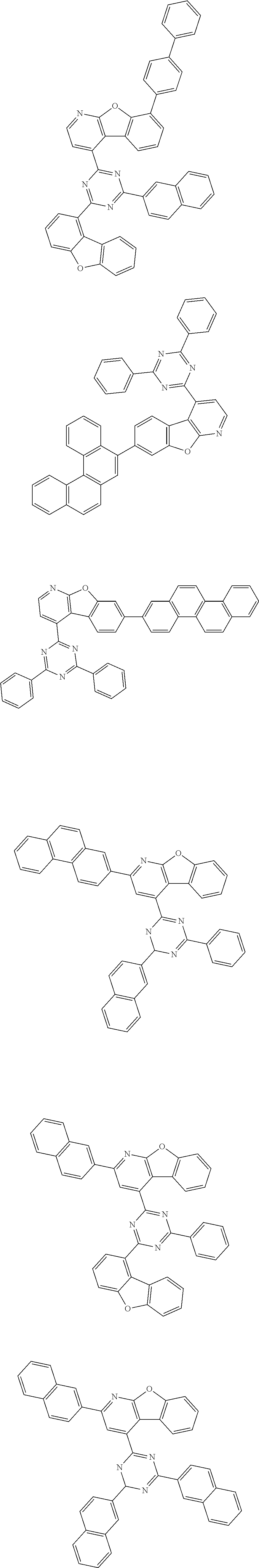

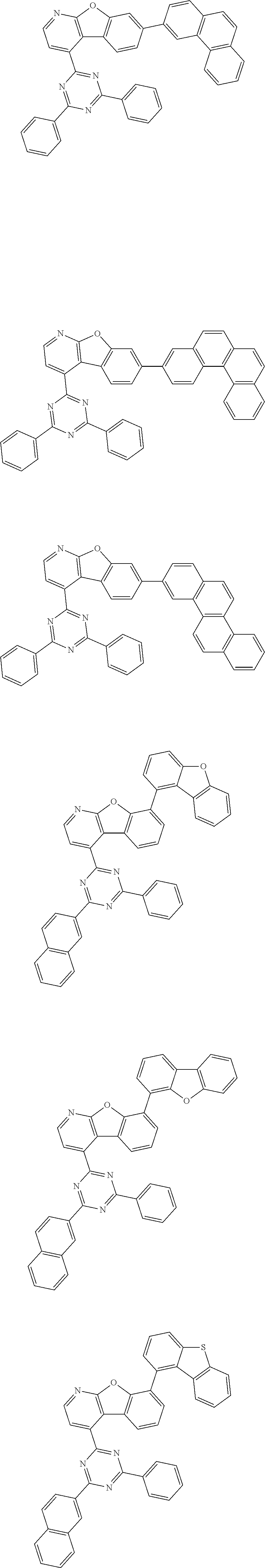 [SMILES: c1ccc(-c2nc(-c3ccccc3)nc(-c3ccnc4oc5cc(-c6ccc7ccc8ccccc8c7c6)ccc5c34)n2)cc1 ; c1ccc(-c2nc(-c3ccccc3)nc(-c3ccnc4oc5cc(-c6ccc7c(ccc8ccc9ccccc9c87)c6)ccc5c34)n2)cc1 ; c1ccc(-c2nc(-c3ccccc3)nc(-c3ccnc4oc5cc(-c6ccc7ccc8c9ccccc9ccc8c7c6)ccc5c34)n2)cc1 ; c1ccc(-c2nc(-c3ccc4ccccc4c3)nc(-c3ccnc4oc5c(-c6cccc7oc8ccccc8c67)cccc5c34)n2)cc1 ; c1ccc(-c2nc(-c3ccc4ccccc4c3)nc(-c3ccnc4oc5c(-c6cccc7c6oc6ccccc67)cccc5c34)n2)cc1 ; c1ccc(-c2nc(-c3ccc4ccccc4c3)nc(-c3ccnc4oc5c(-c6cccc7sc8ccccc8c67)cccc5c34)n2)cc1]

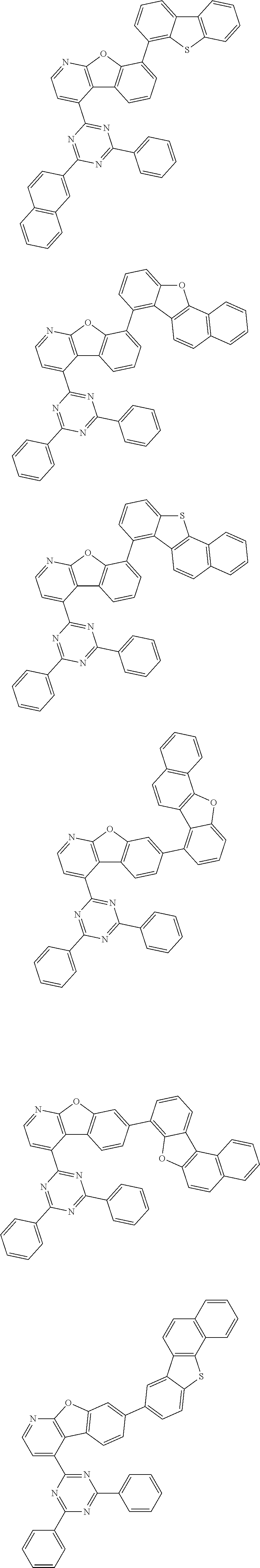 [SMILES: c1ccc(-c2nc(-c3ccc4ccccc4c3)nc(-c3ccnc4oc5c(-c6cccc7c6sc6ccccc67)cccc5c34)n2)cc1 ; c1ccc(-c2nc(-c3ccccc3)nc(-c3ccnc4oc5c(-c6cccc7oc8c9ccccc9ccc8c67)cccc5c34)n2)cc1 ; c1ccc(-c2nc(-c3ccccc3)nc(-c3ccnc4oc5c(-c6cccc7sc8c9ccccc9ccc8c67)cccc5c34)n2)cc1 ; c1ccc(-c2nc(-c3ccccc3)nc(-c3ccnc4oc5cc(-c6cccc7oc8c9ccccc9ccc8c67)ccc5c34)n2)cc1 ; c1ccc(-c2nc(-c3ccccc3)nc(-c3ccnc4oc5cc(-c6cccc7c6oc6ccc8ccccc8c67)ccc5c34)n2)cc1 ; c1ccc(-c2nc(-c3ccccc3)nc(-c3ccnc4oc5cc(-c6ccc7sc8c9ccccc9ccc8c7c6)ccc5c34)n2)cc1]

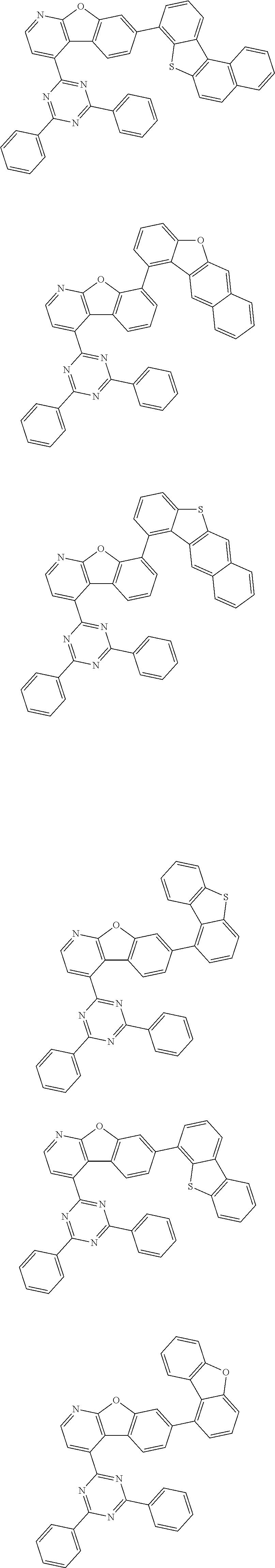 [SMILES: c1ccc(-c2nc(-c3ccccc3)nc(-c3ccnc4oc5cc(-c6cccc7c6sc6ccc8ccccc8c67)ccc5c34)n2)cc1 ; c1ccc(-c2nc(-c3ccccc3)nc(-c3ccnc4oc5c(-c6cccc7oc8cc9ccccc9cc8c67)cccc5c34)n2)cc1 ; c1ccc(-c2nc(-c3ccccc3)nc(-c3ccnc4oc5c(-c6cccc7sc8cc9ccccc9cc8c67)cccc5c34)n2)cc1 ; c1ccc(-c2nc(-c3ccccc3)nc(-c3ccnc4oc5cc(-c6cccc7sc8ccccc8c67)ccc5c34)n2)cc1 ; c1ccc(-c2nc(-c3ccccc3)nc(-c3ccnc4oc5cc(-c6cccc7c6sc6ccccc67)ccc5c34)n2)cc1 ; c1ccc(-c2nc(-c3ccccc3)nc(-c3ccnc4oc5cc(-c6cccc7oc8ccccc8c67)ccc5c34)n2)cc1]

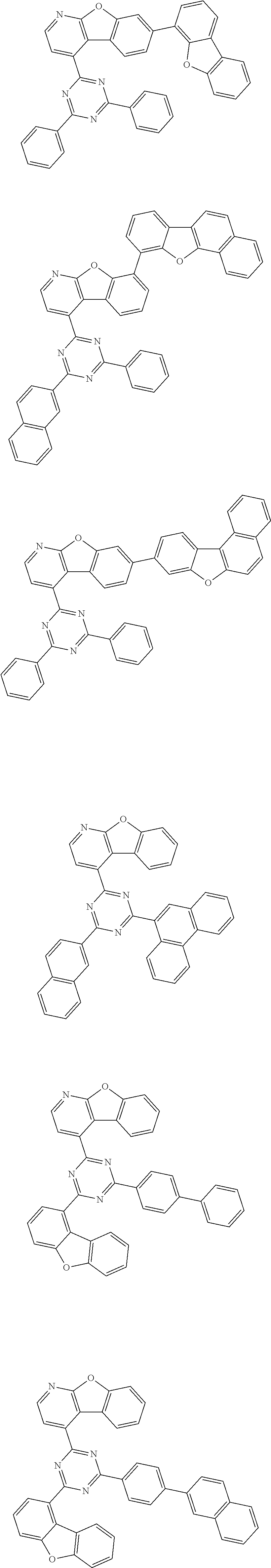 [SMILES: c1ccc(-c2nc(-c3ccccc3)nc(-c3ccnc4oc5cc(-c6cccc7c6oc6ccccc67)ccc5c34)n2)cc1 ; c1ccc(-c2nc(-c3ccc4ccccc4c3)nc(-c3ccnc4oc5c(-c6cccc7c6oc6c8ccccc8ccc76)cccc5c34)n2)cc1 ; c1ccc(-c2nc(-c3ccccc3)nc(-c3ccnc4oc5cc(-c6ccc7c(c6)oc6ccc8ccccc8c67)ccc5c34)n2)cc1 ; c1ccc2cc(-c3nc(-c4cc5ccccc5c5ccccc45)nc(-c4ccnc5oc6ccccc6c45)n3)ccc2c1 ; c1ccc(-c2ccc(-c3nc(-c4cccc5oc6ccccc6c45)nc(-c4ccnc5oc6ccccc6c45)n3)cc2)cc1 ; c1ccc2cc(-c3ccc(-c4nc(-c5cccc6oc7ccccc7c56)nc(-c5ccnc6oc7ccccc7c56)n4)cc3)ccc2c1]

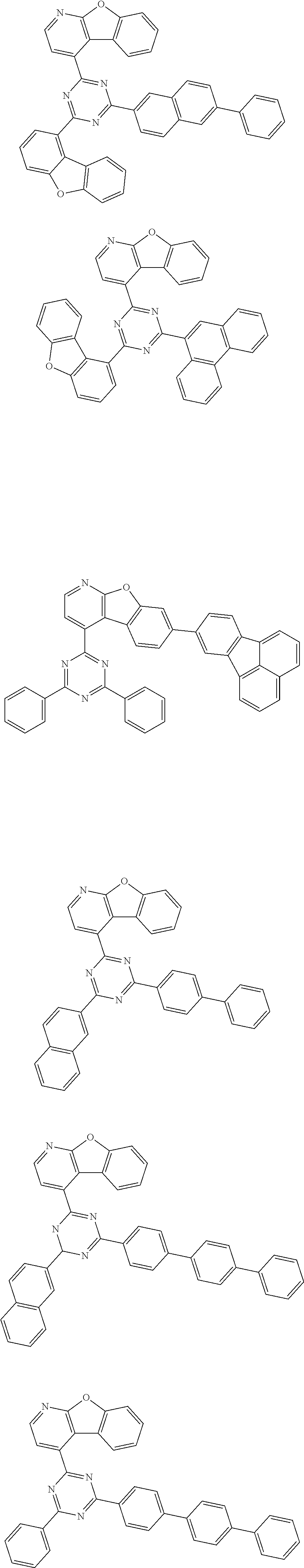

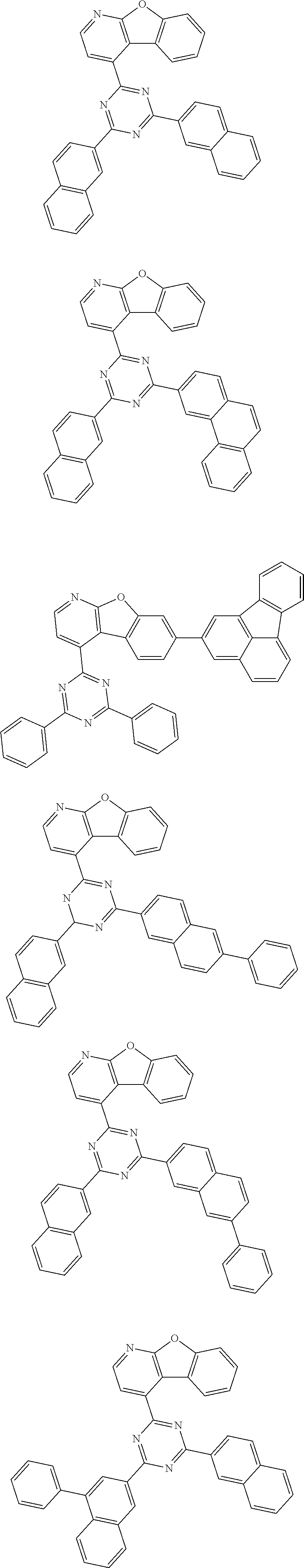

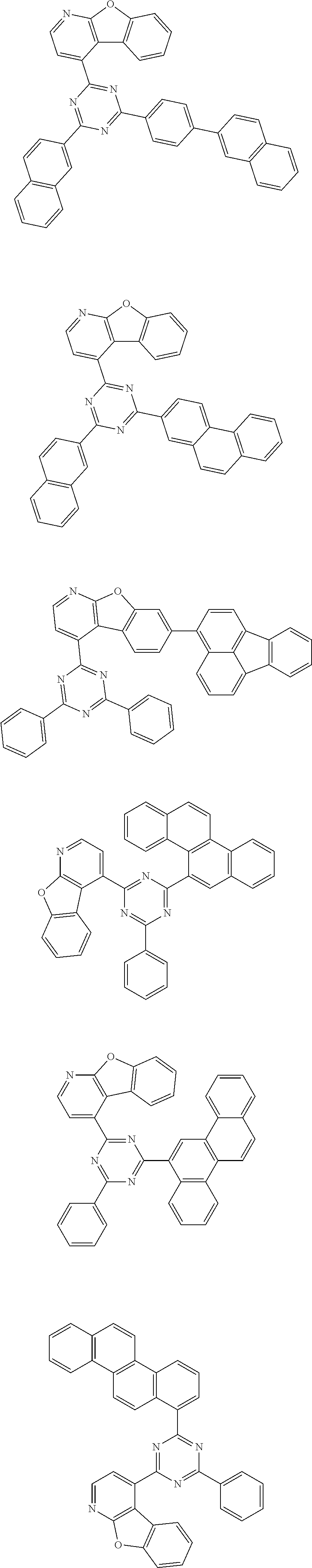 [SMILES: c1ccc2cc(-c3ccc(-c4nc(-c5ccc6ccccc6c5)nc(-c5ccnc6oc7ccccc7c56)n4)cc3)ccc2c1 ; c1ccc2cc(-c3nc(-c4ccc5c(ccc6ccccc65)c4)nc(-c4ccnc5oc6ccccc6c45)n3)ccc2c1 ; c1ccc(-c2nc(-c3ccccc3)nc(-c3ccnc4oc5cc(-c6ccc7c8c(cccc68)-c6ccccc6-7)ccc5c34)n2)cc1 ; c1ccc(-c2nc(-c3ccnc4oc5ccccc5c34)nc(-c3cc4ccccc4c4ccc5ccccc5c34)n2)cc1 ; c1ccc(-c2nc(-c3cc4c5ccccc5ccc4c4ccccc34)nc(-c3ccnc4oc5ccccc5c34)n2)cc1 ; c1ccc(-c2nc(-c3cccc4c3ccc3c5ccccc5ccc43)nc(-c3ccnc4oc5ccccc5c34)n2)cc1]

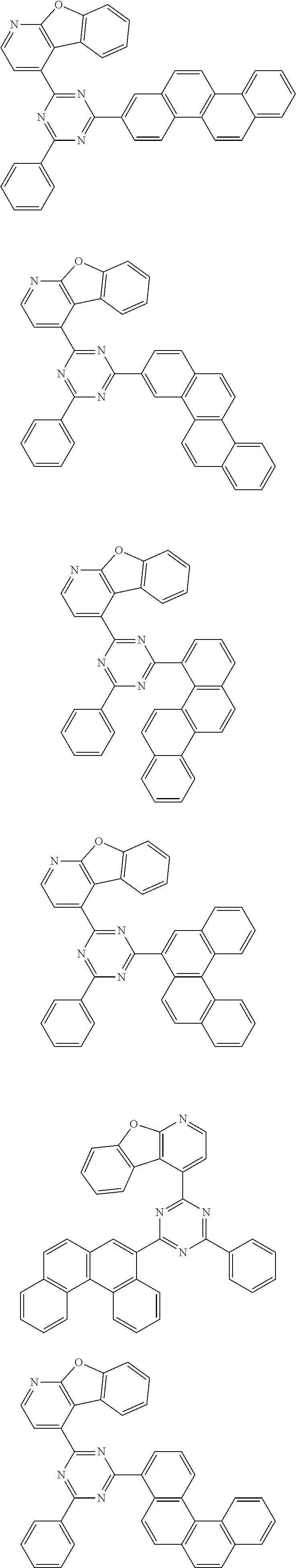

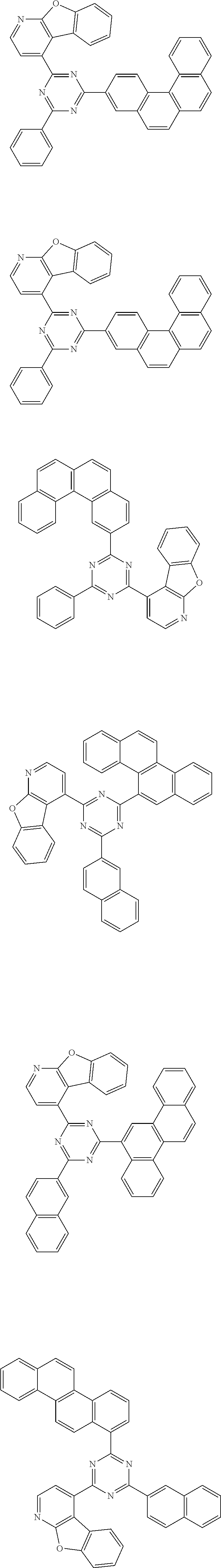 [SMILES: c1ccc(-c2nc(-c3ccc4c(ccc5ccc6ccccc6c54)c3)nc(-c3ccnc4oc5ccccc5c34)n2)cc1 ; c1ccc(-c2nc(-c3ccc4c(ccc5ccc6ccccc6c54)c3)nc(-c3ccnc4oc5ccccc5c34)n2)cc1 ; c1ccc(-c2nc(-c3ccc4ccc5ccc6ccccc6c5c4c3)nc(-c3ccnc4oc5ccccc5c34)n2)cc1 ; c1ccc2cc(-c3nc(-c4ccnc5oc6ccccc6c45)nc(-c4cc5ccccc5c5ccc6ccccc6c45)n3)ccc2c1 ; c1ccc2cc(-c3nc(-c4cc5c6ccccc6ccc5c5ccccc45)nc(-c4ccnc5oc6ccccc6c45)n3)ccc2c1 ; c1ccc2cc(-c3nc(-c4cccc5c4ccc4c6ccccc6ccc54)nc(-c4ccnc5oc6ccccc6c45)n3)ccc2c1]

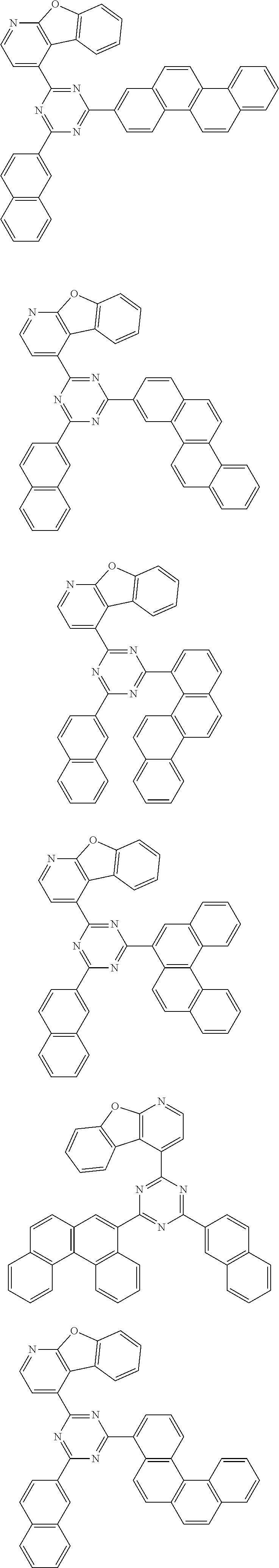

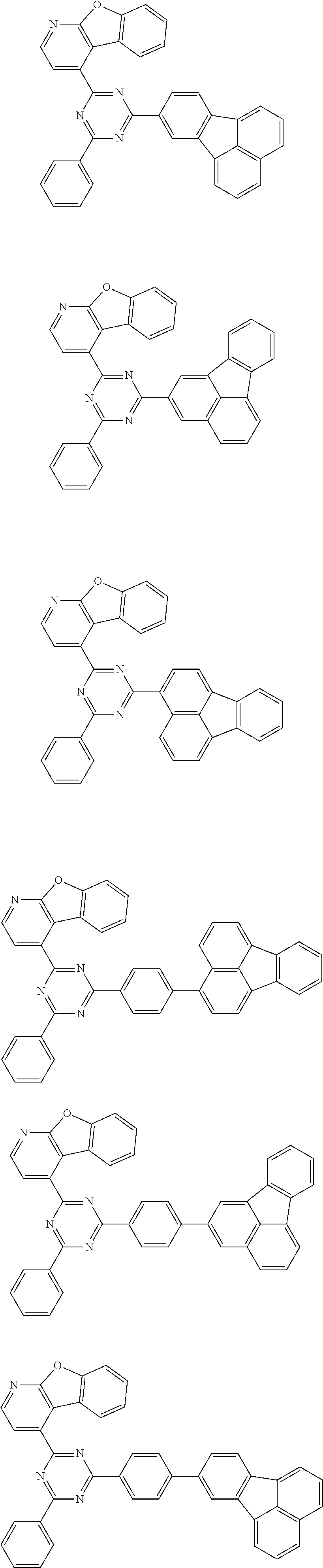 [SMILES: c1ccc(-c2nc(-c3ccc4c(c3)-c3cccc5cccc-4c35)nc(-c3ccnc4oc5ccccc5c34)n2)cc1 ; c1ccc(-c2nc(-c3cc4c5c(cccc5c3)-c3ccccc3-4)nc(-c3ccnc4oc5ccccc5c34)n2)cc1 ; c1ccc(-c2nc(-c3ccc4c5c(cccc35)-c3ccccc3-4)nc(-c3ccnc4oc5ccccc5c34)n2)cc1 ; c1ccc(-c2nc(-c3ccc(-c4ccc5c6c(cccc46)-c4ccccc4-5)cc3)nc(-c3ccnc4oc5ccccc5c34)n2)cc1 ; c1ccc(-c2nc(-c3ccc(-c4cc5c6c(cccc6c4)-c4ccccc4-5)cc3)nc(-c3ccnc4oc5ccccc5c34)n2)cc1 ; c1ccc(-c2nc(-c3ccc(-c4ccc5c(c4)-c4cccc6cccc-5c46)cc3)nc(-c3ccnc4oc5ccccc5c34)n2)cc1]

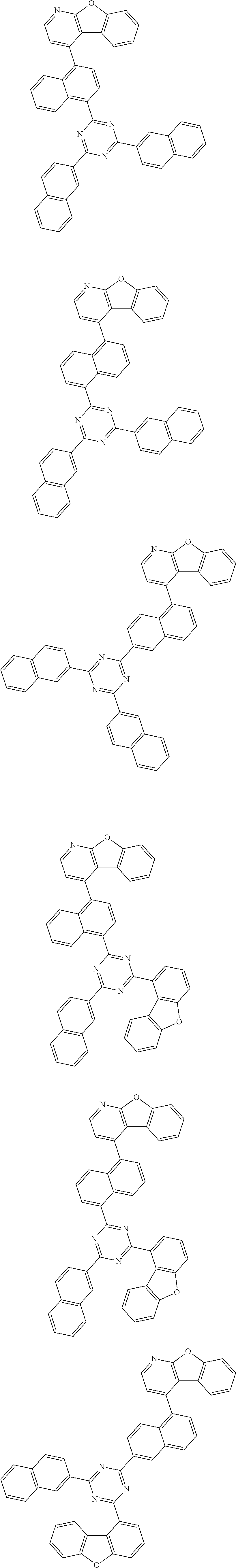

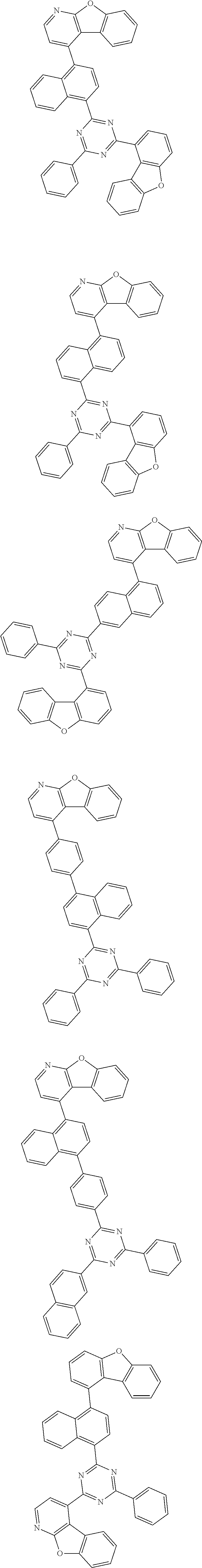

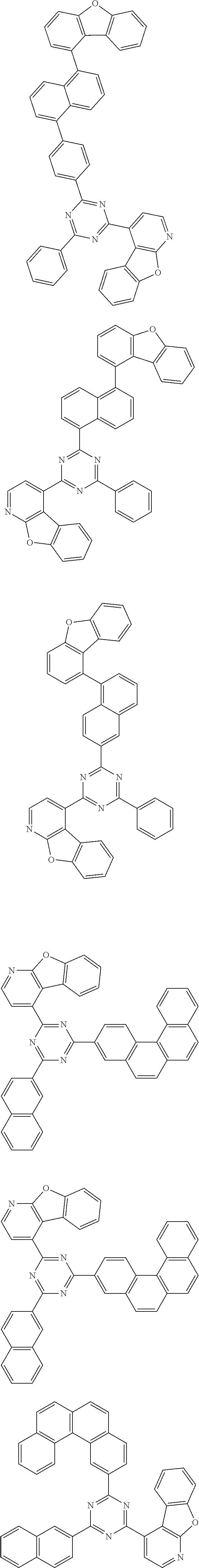

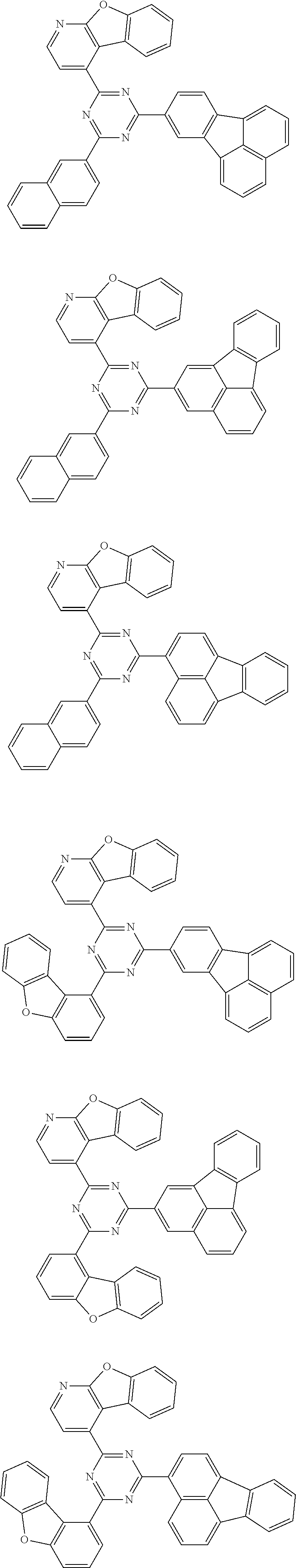 [SMILES: c1ccc2cc(-c3nc(-c4ccc5c(c4)-c4cccc6cccc-5c46)nc(-c4ccnc5oc6ccccc6c45)n3)ccc2c1 ; c1ccc2c(c1)-c1cccc3cc(-c4nc(-c5ccc6ccccc6c5)nc(-c5ccnc6oc7ccccc7c56)n4)cc-2c13 ; c1ccc2c(c1)-c1cccc3c(-c4nc(-c5ccc6ccccc6c5)nc(-c5ccnc6oc7ccccc7c56)n4)ccc-2c13 ; c1cc2c3c(cccc3c1)-c1cc(-c3nc(-c4cccc5oc6ccccc6c45)nc(-c4ccnc5oc6ccccc6c45)n3)ccc1-2 ; c1ccc2c(c1)-c1cccc3cc(-c4nc(-c5cccc6oc7ccccc7c56)nc(-c5ccnc6oc7ccccc7c56)n4)cc-2c13 ; c1ccc2c(c1)-c1cccc3c(-c4nc(-c5cccc6oc7ccccc7c56)nc(-c5ccnc6oc7ccccc7c56)n4)ccc-2c13]

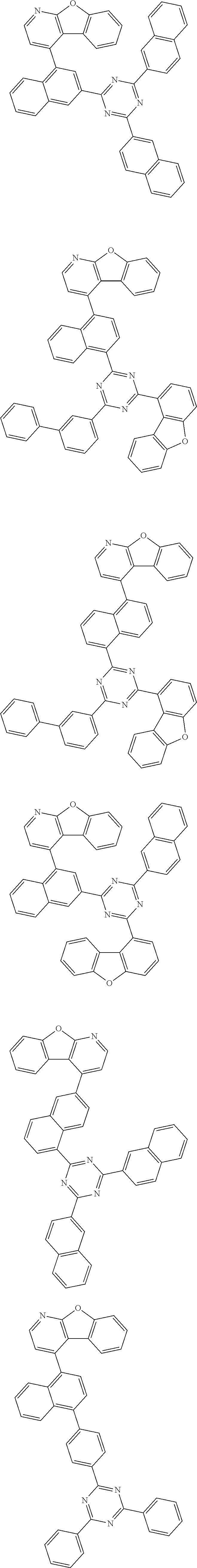

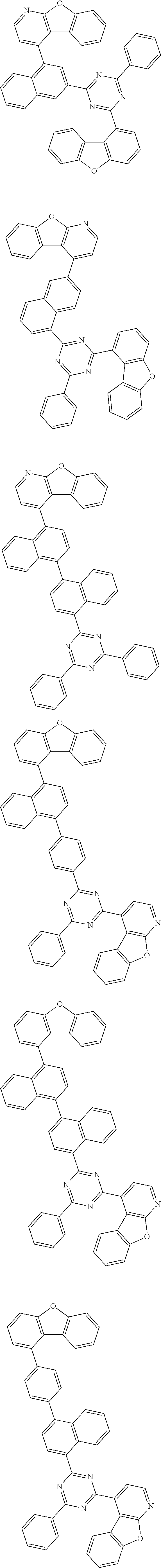

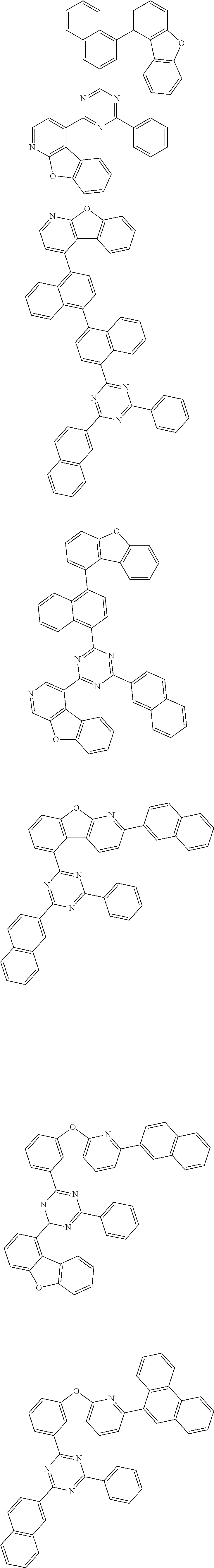

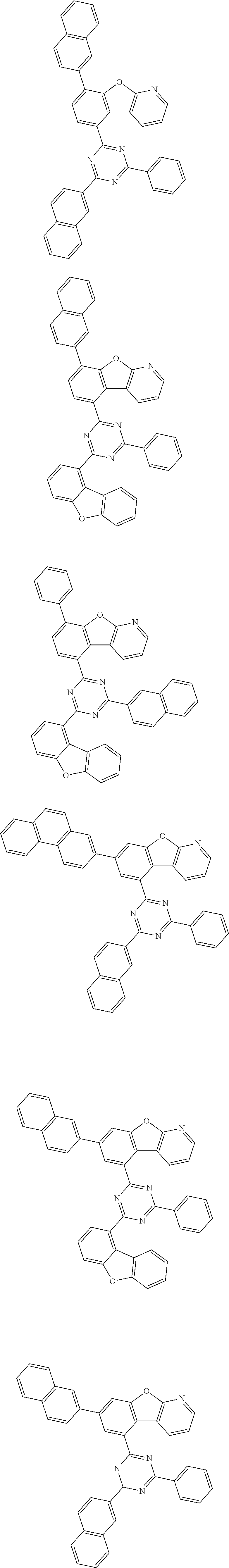

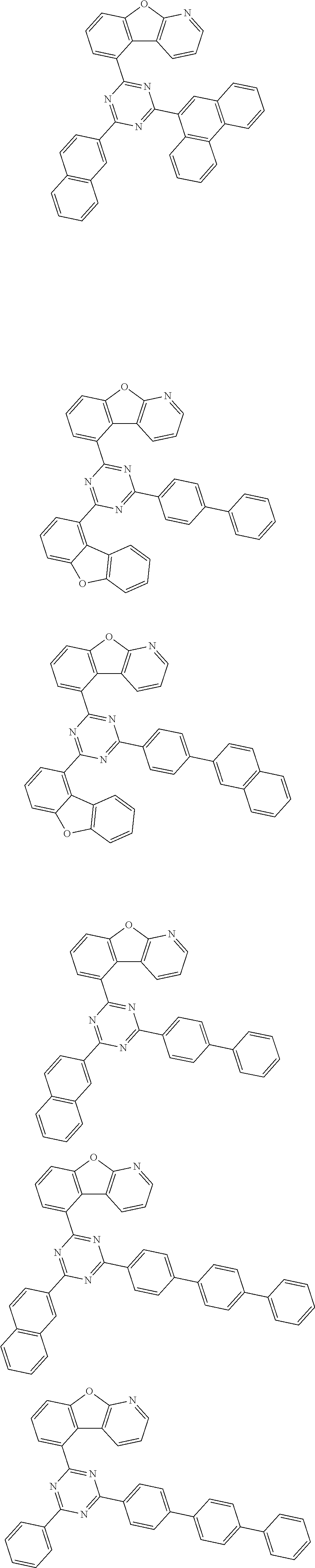

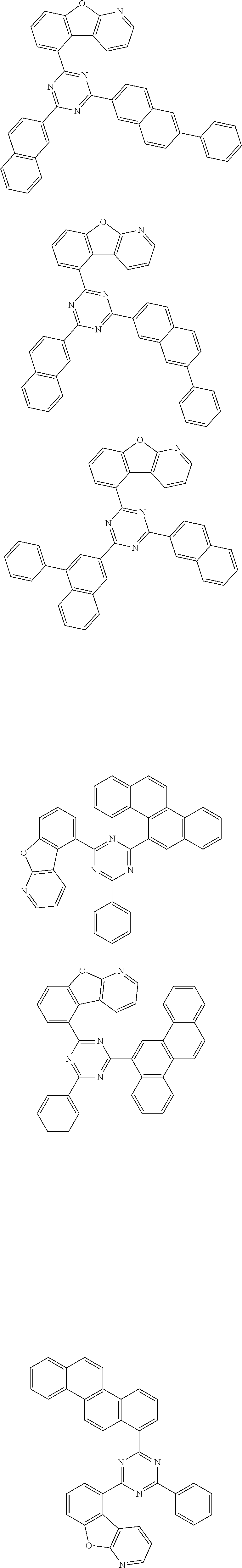

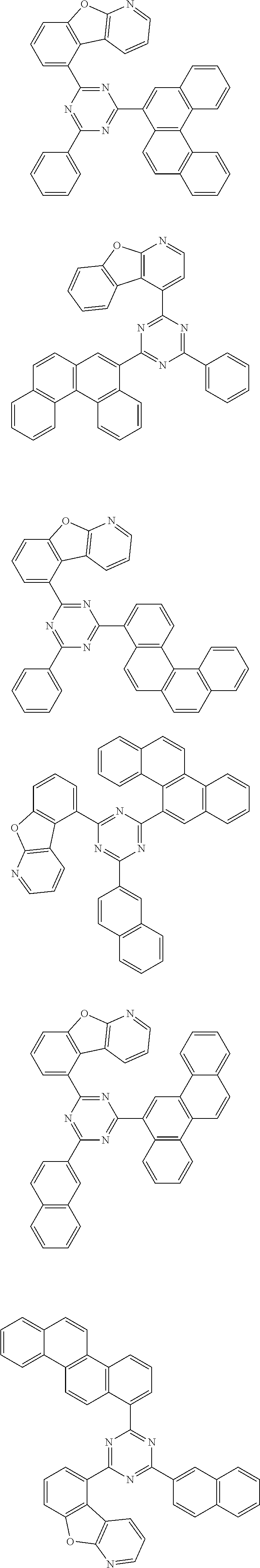

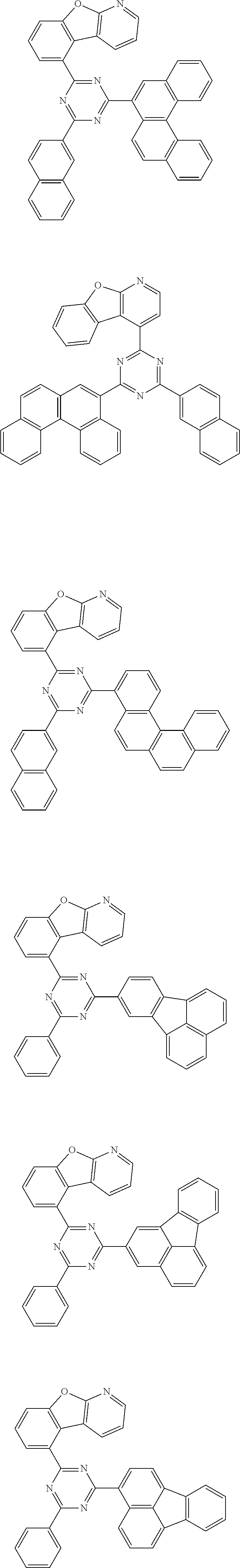 [SMILES: c1ccc2cc(-c3nc(-c4cc5ccccc5c5c4ccc4ccccc45)nc(-c4cccc5oc6ncccc6c45)n3)ccc2c1 ; c1ccc2cc(-c3nc(-c4cc5ccc6ccccc6c5c5ccccc45)nc(-c4ccnc5oc6ccccc6c45)n3)ccc2c1 ; c1ccc2cc(-c3nc(-c4cccc5c4ccc4ccc6ccccc6c45)nc(-c4cccc5oc6ncccc6c45)n3)ccc2c1 ; c1ccc(-c2nc(-c3ccc4c(c3)-c3cccc5cccc-4c35)nc(-c3cccc4oc5ncccc5c34)n2)cc1 ; c1ccc(-c2nc(-c3cc4c5c(cccc5c3)-c3ccccc3-4)nc(-c3cccc4oc5ncccc5c34)n2)cc1 ; c1ccc(-c2nc(-c3ccc4c5c(cccc35)-c3ccccc3-4)nc(-c3cccc4oc5ncccc5c34)n2)cc1]

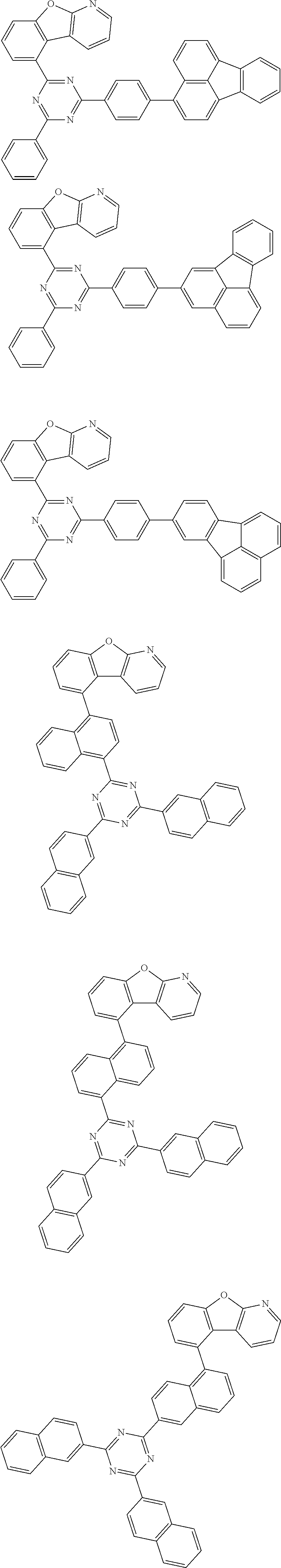

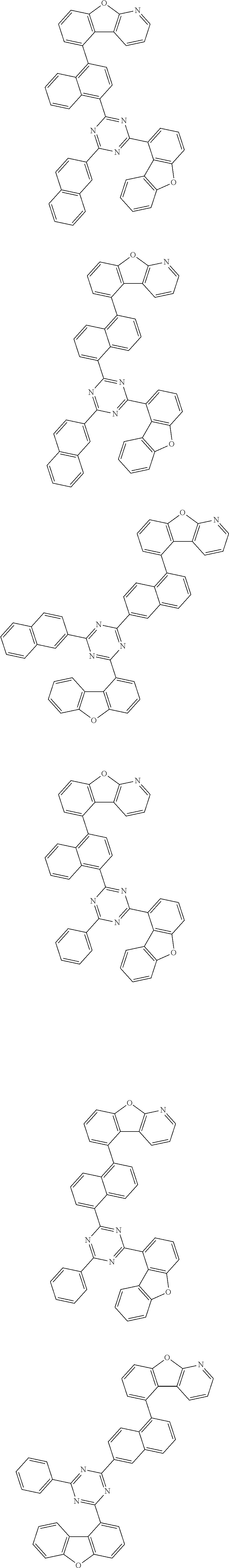

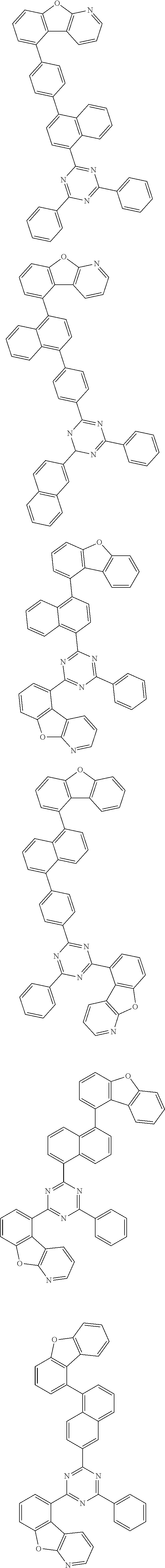

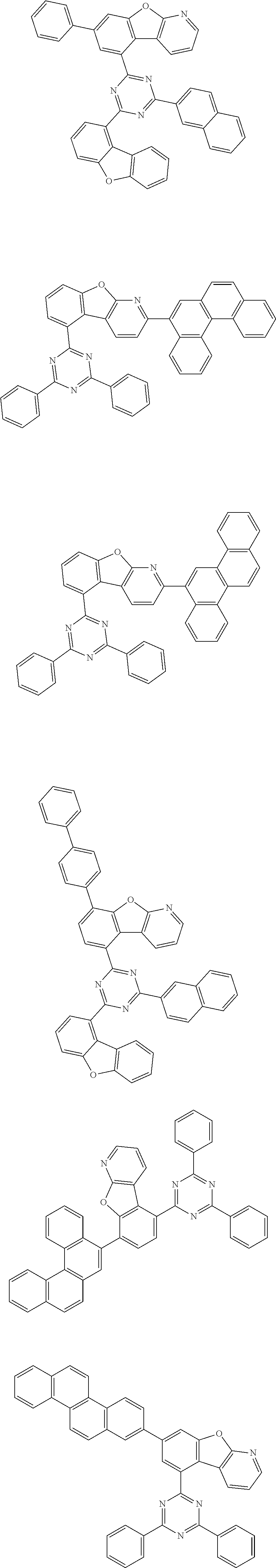

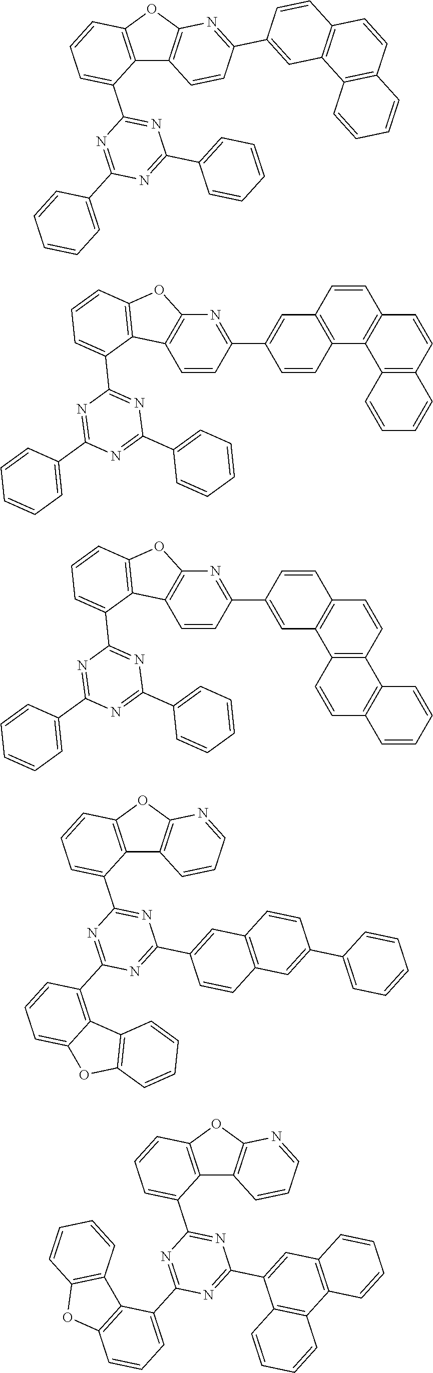

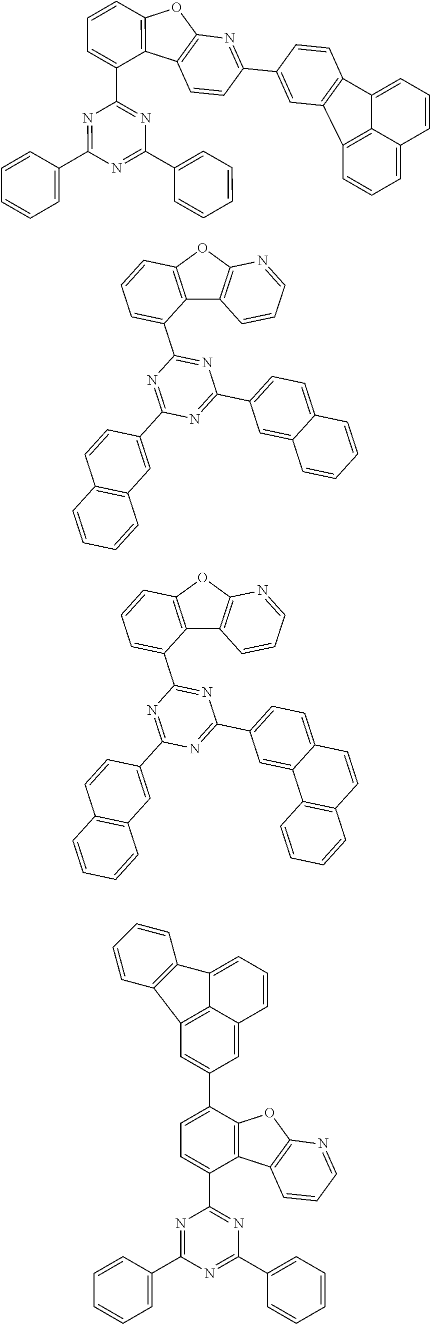

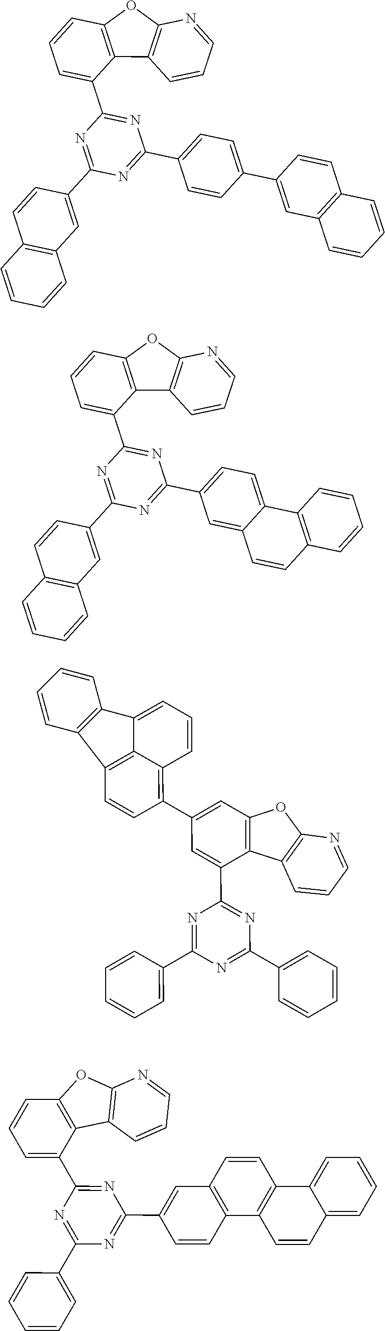

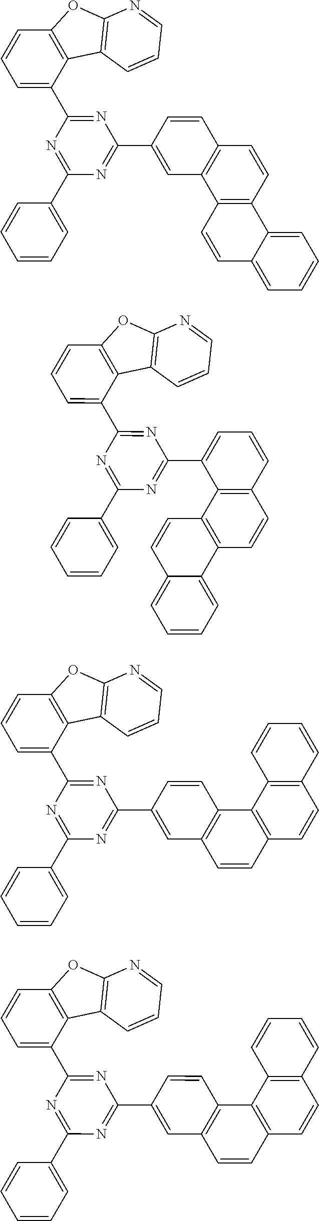

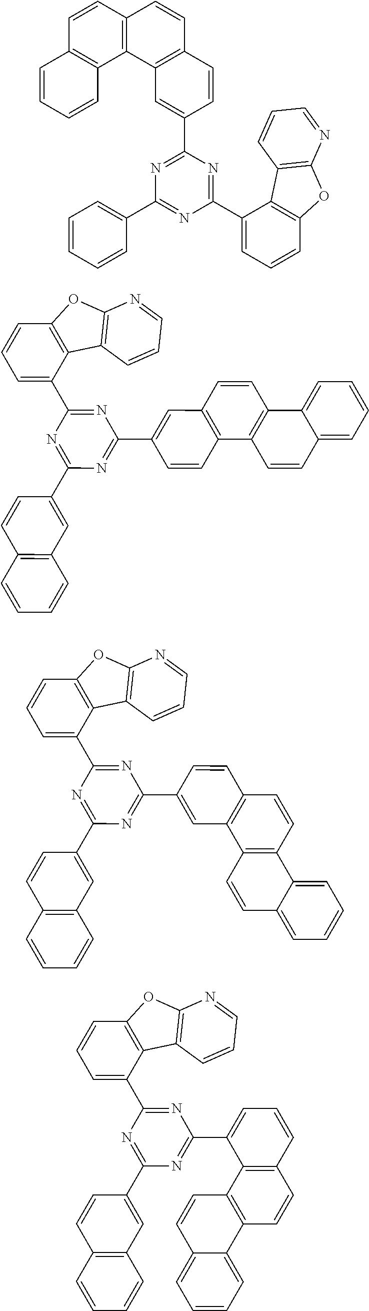

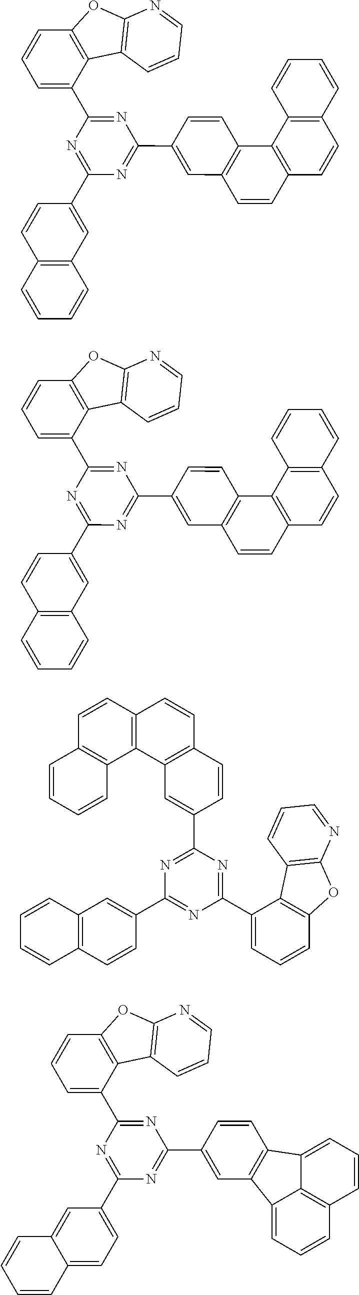

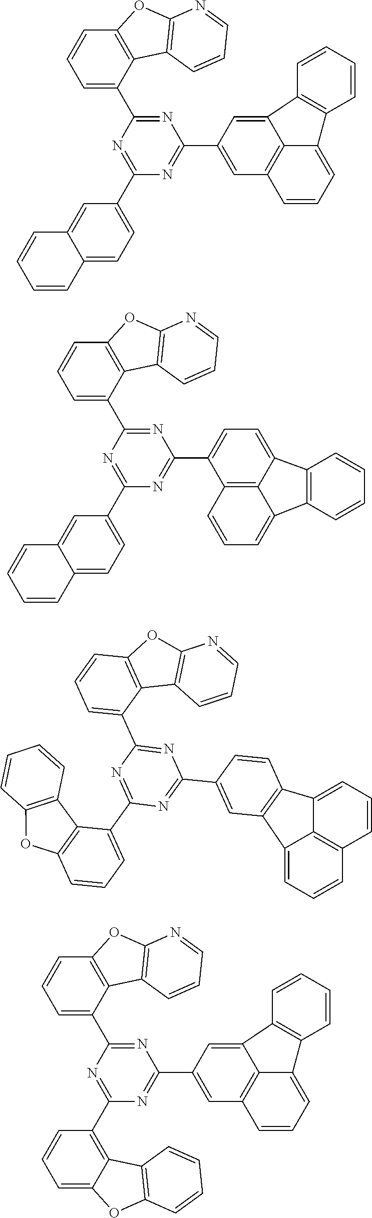

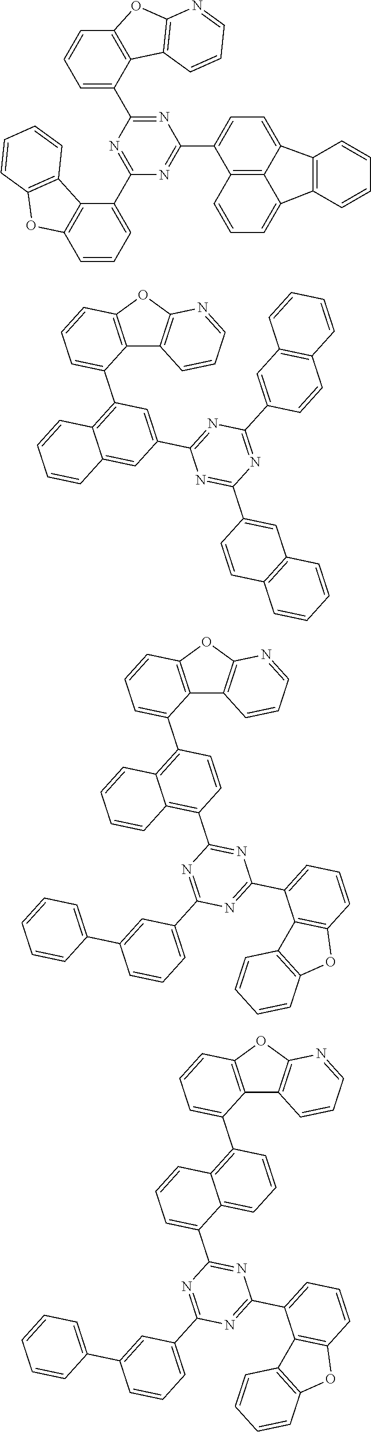

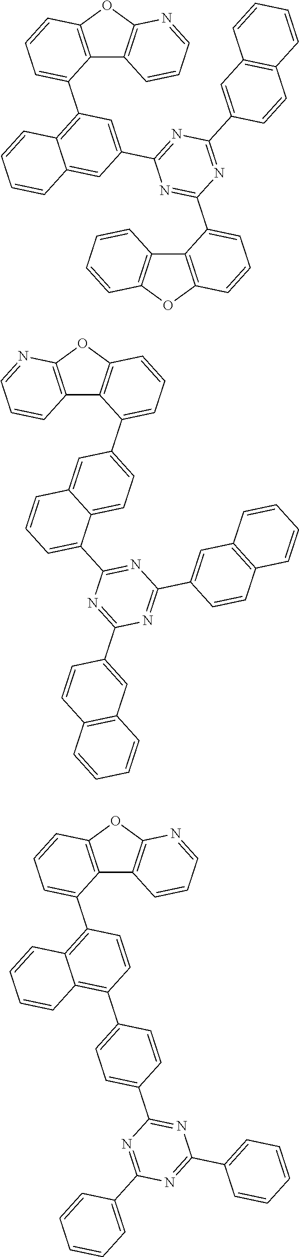

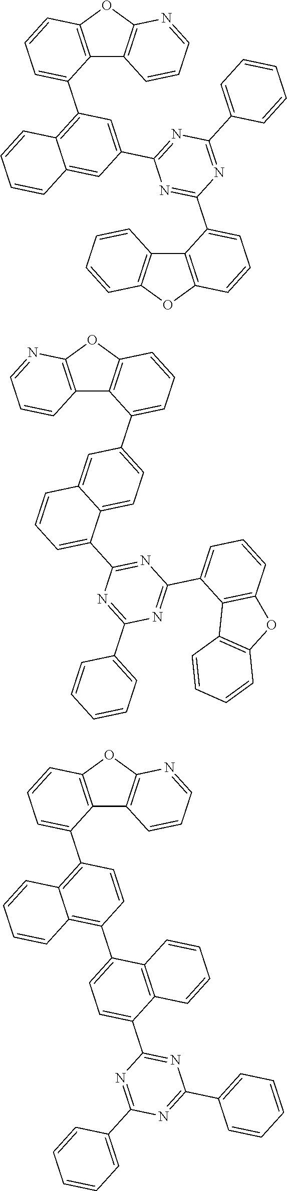

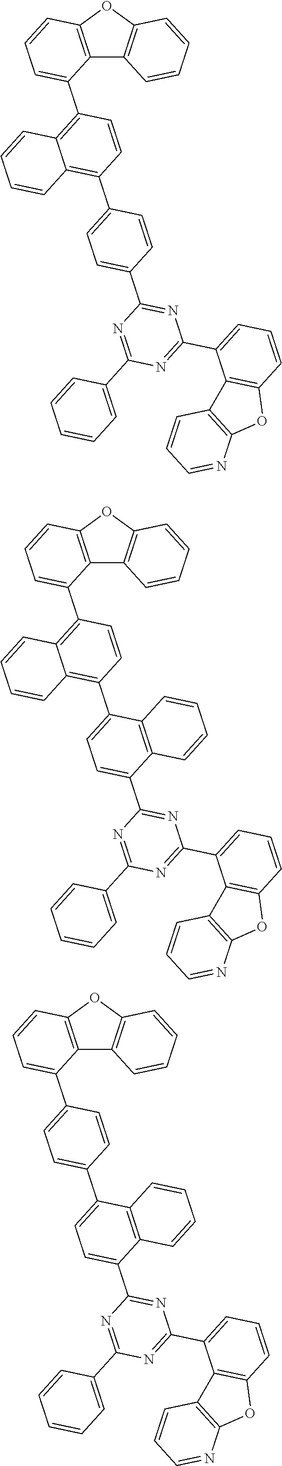

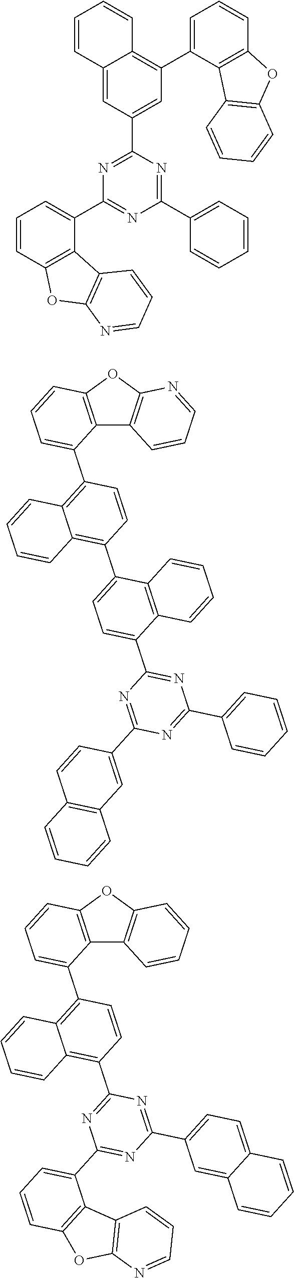

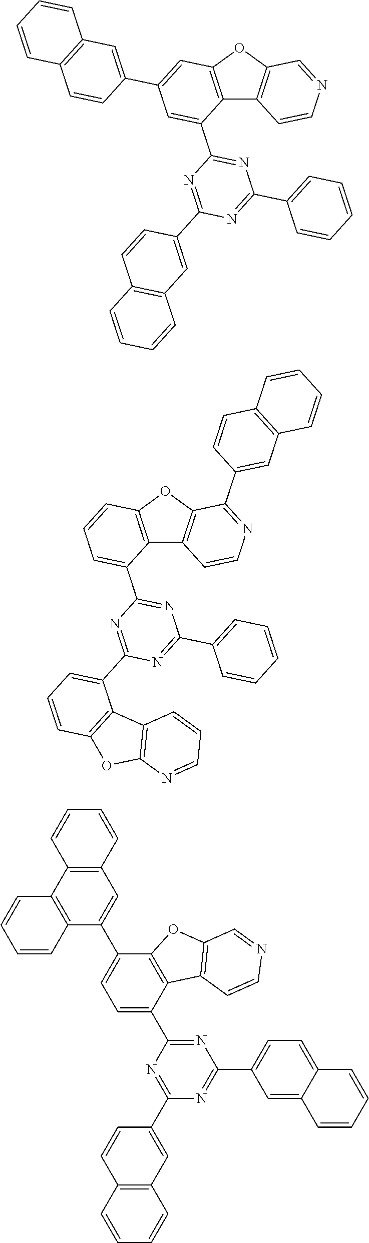

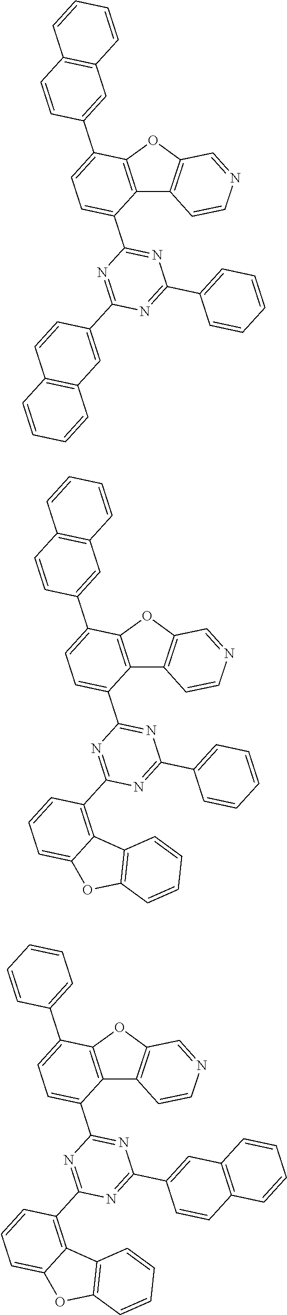

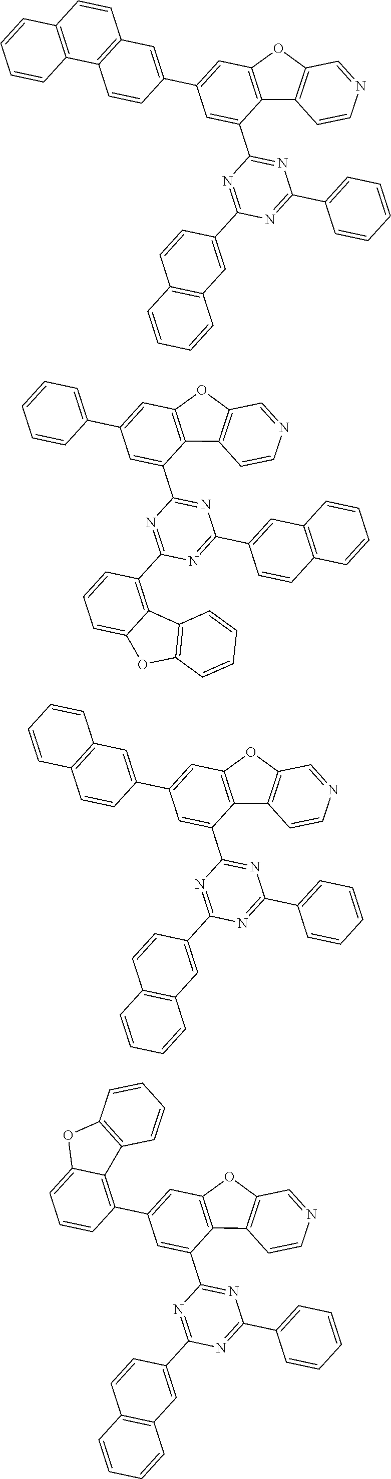

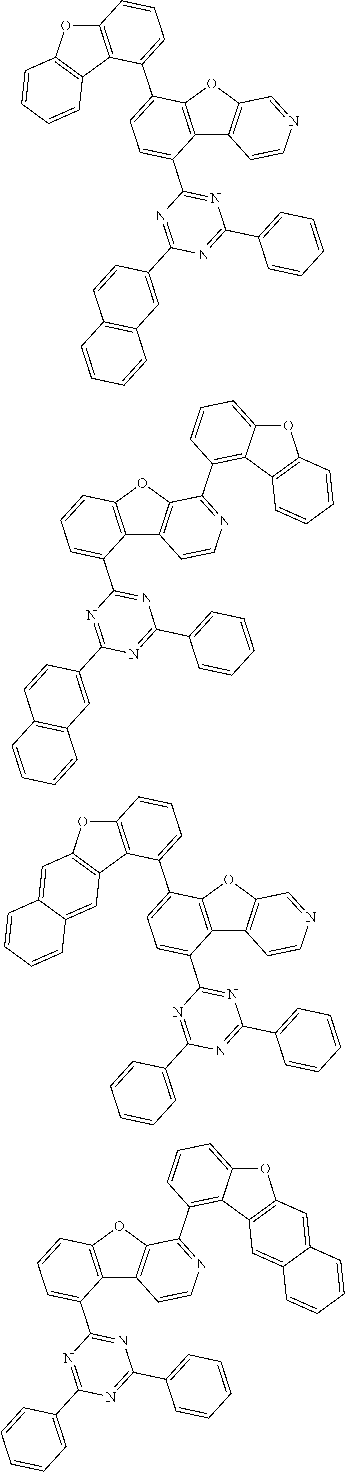

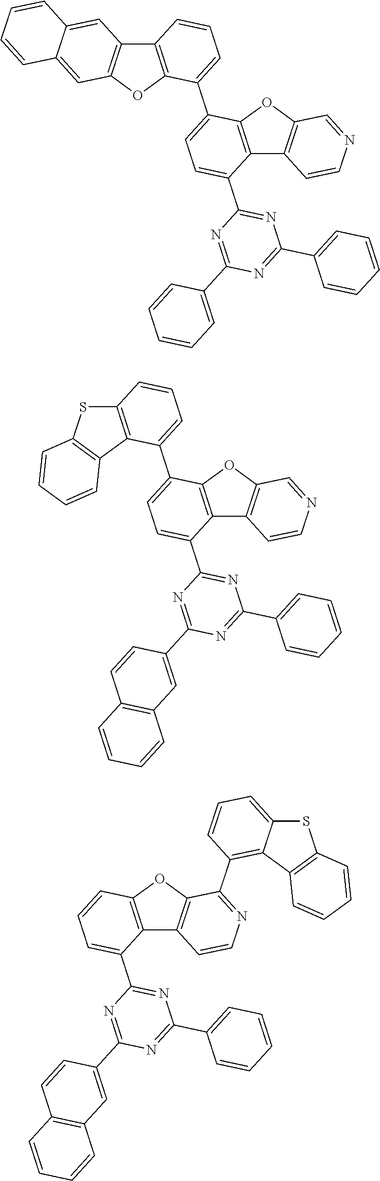

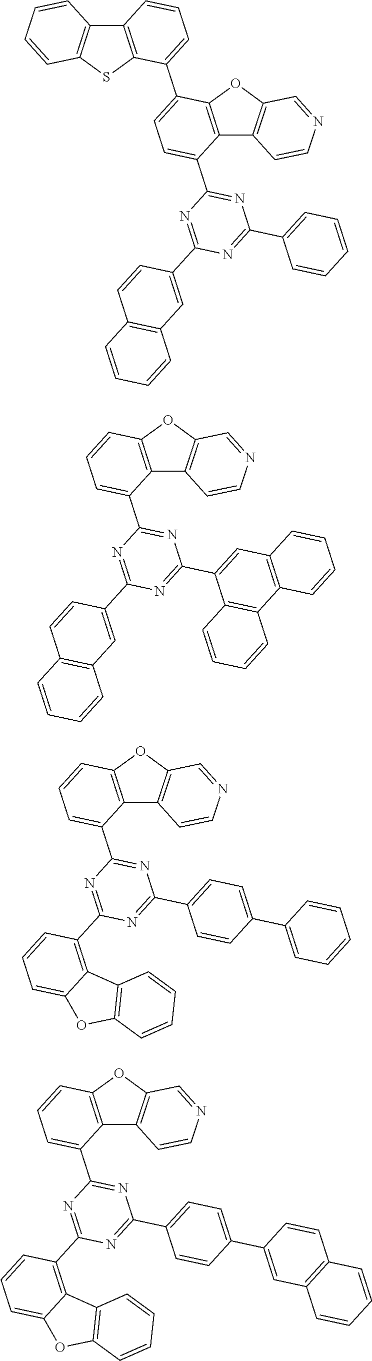

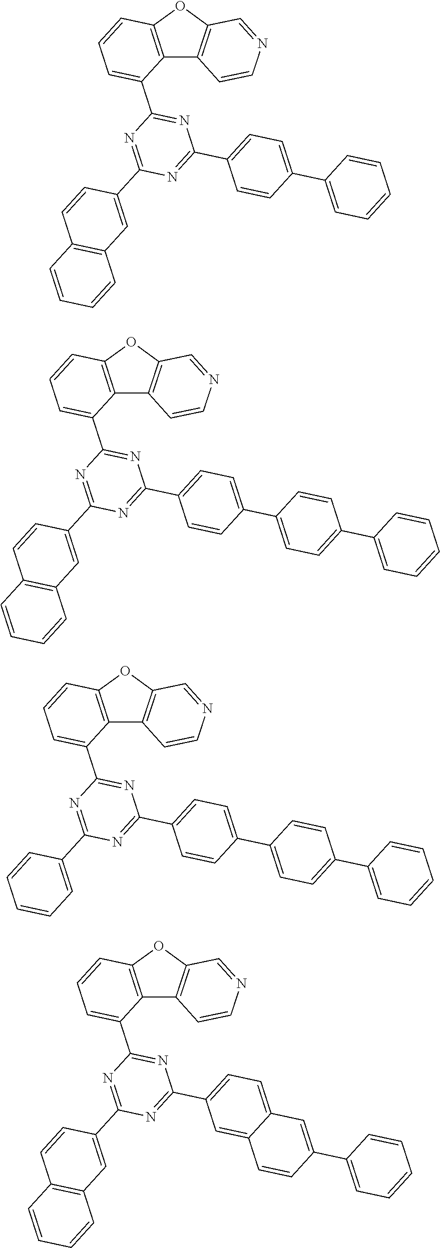

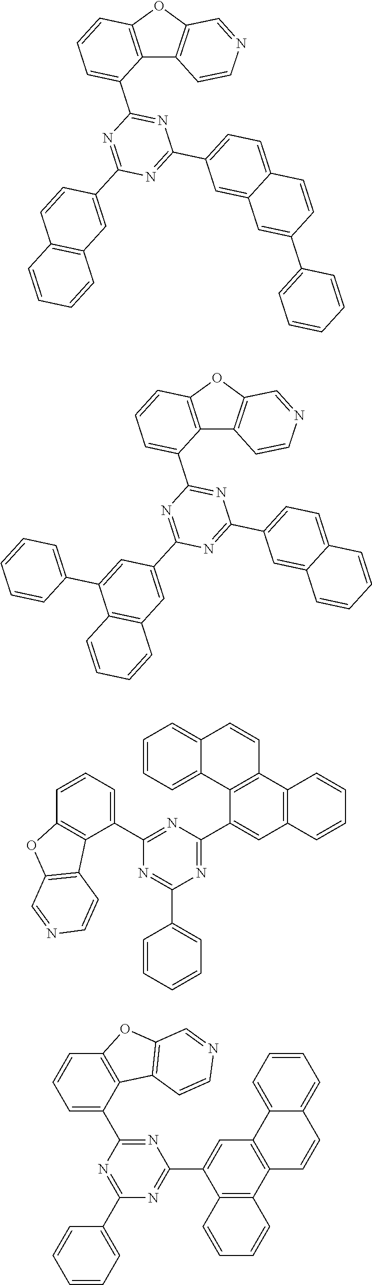

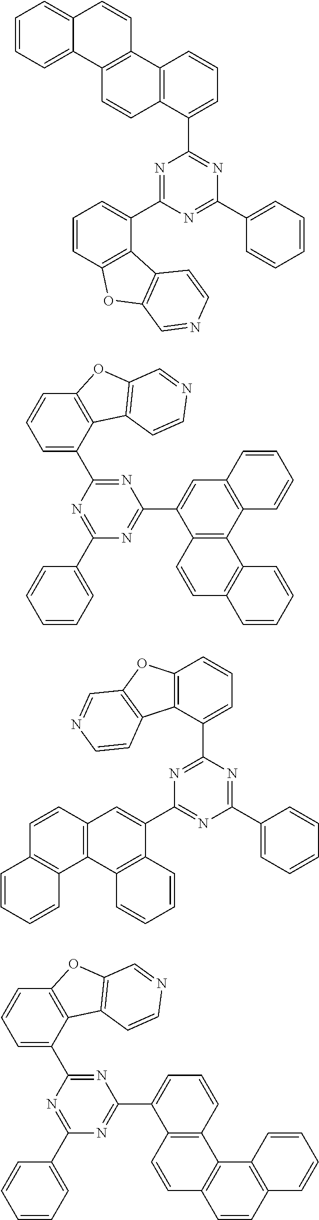

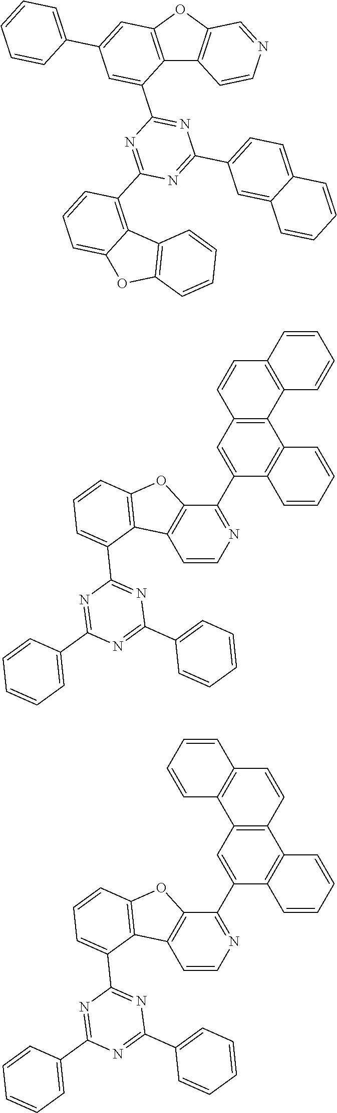 [SMILES: c1ccc(-c2cc(-c3nc(-c4ccc5ccccc5c4)nc(-c4cccc5oc6ccccc6c45)n3)c3c(c2)oc2cnccc23)cc1 ; c1ccc(-c2nc(-c3ccccc3)nc(-c3cccc4oc5c(-c6cc7ccc8ccccc8c7c7ccccc67)nccc5c34)n2)cc1 ; c1ccc(-c2nc(-c3ccccc3)nc(-c3cccc4oc5c(-c6cc7c8ccccc8ccc7c7ccccc67)nccc5c34)n2)cc1]

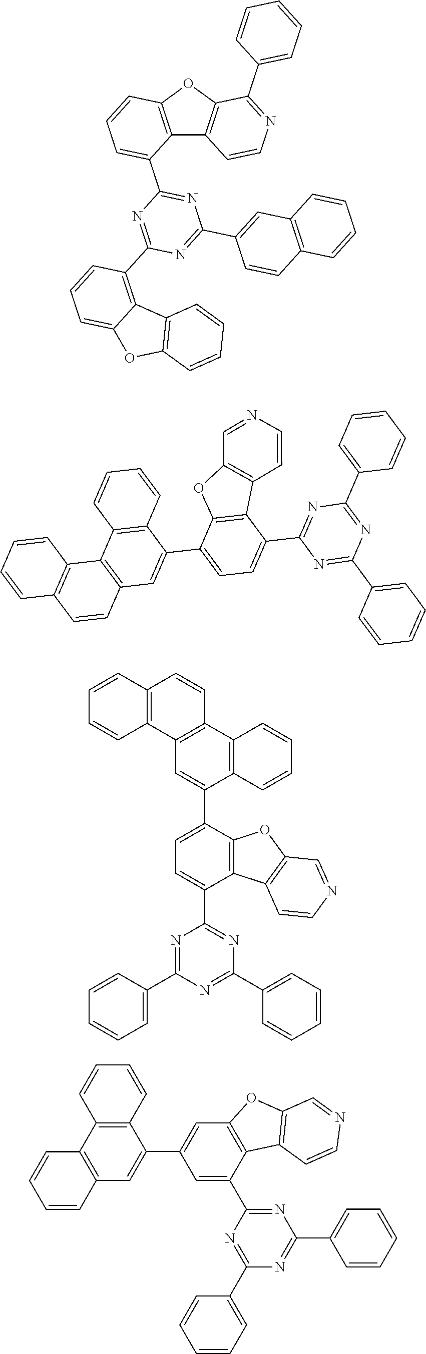

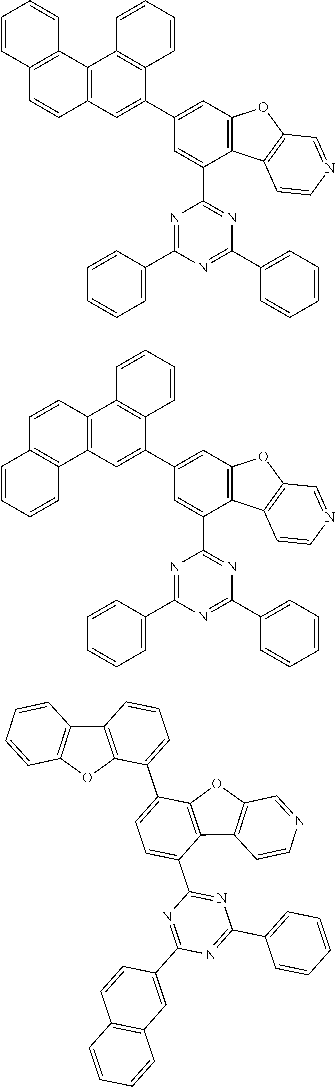

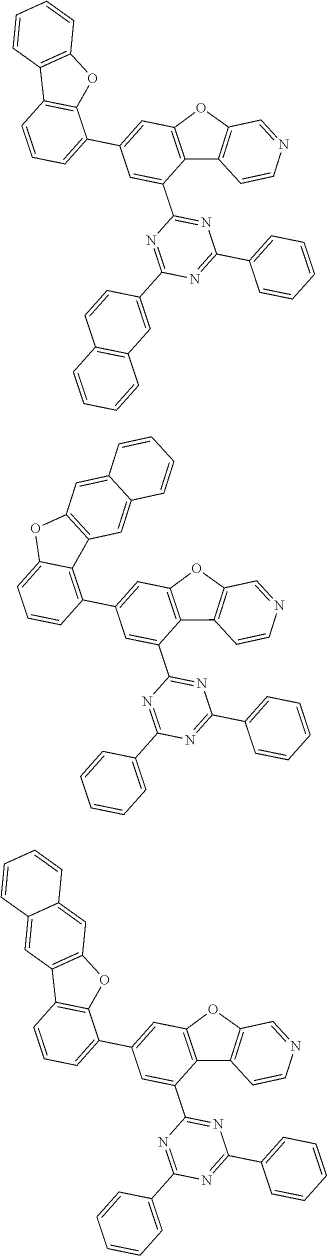

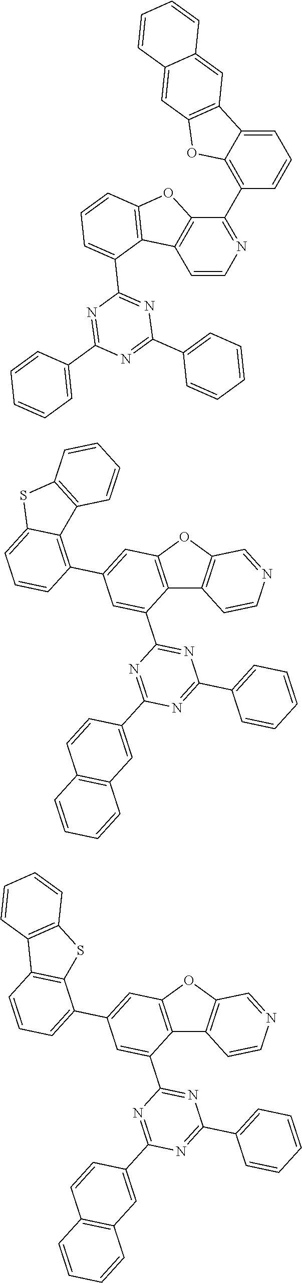

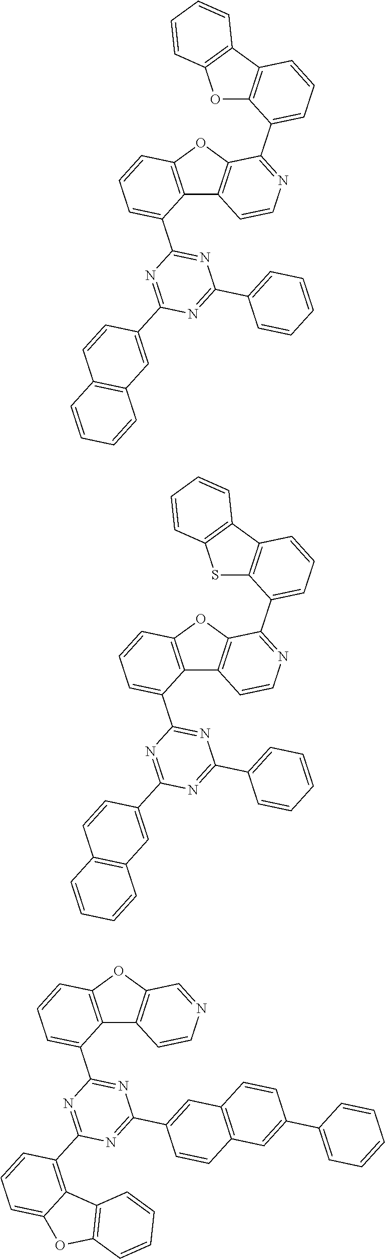 [SMILES: c1ccc(-c2nc(-c3ccc4ccccc4c3)nc(-c3cccc4oc5c(-c6cccc7c6oc6ccccc67)nccc5c34)n2)cc1 ; c1ccc(-c2nc(-c3ccc4ccccc4c3)nc(-c3cccc4oc5c(-c6cccc7c6sc6ccccc67)nccc5c34)n2)cc1 ; c1ccc(-c2ccc3cc(-c4nc(-c5cccc6oc7ccccc7c56)nc(-c5cccc6oc7cnccc7c56)n4)ccc3c2)cc1]

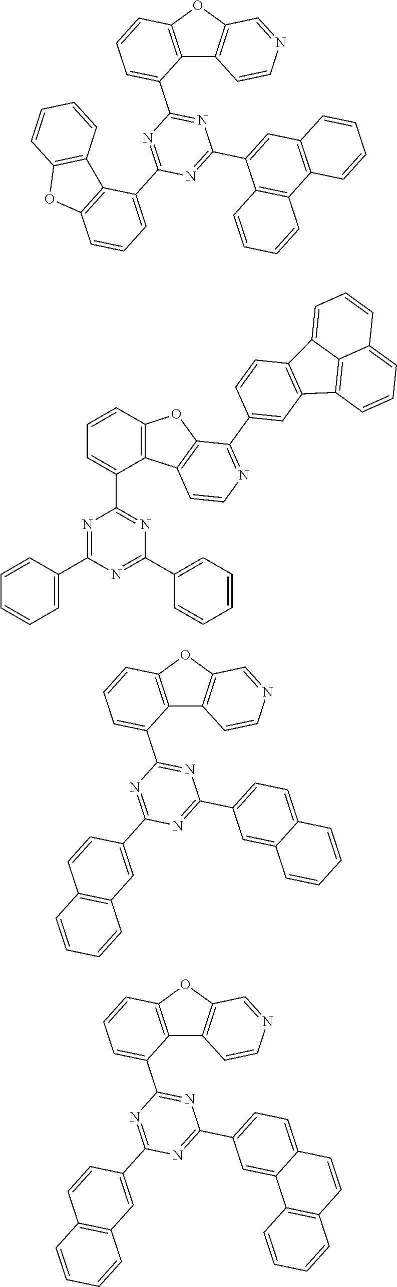

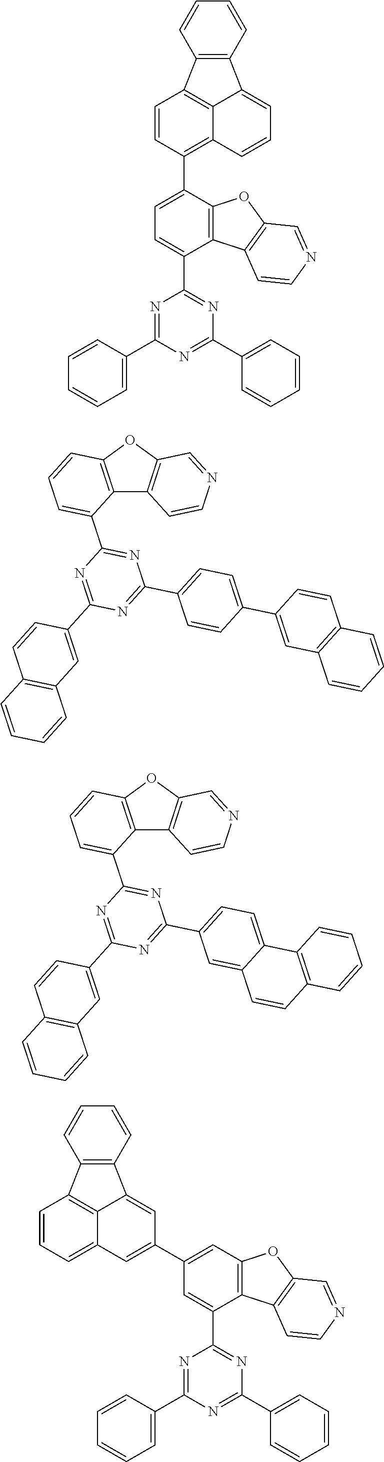

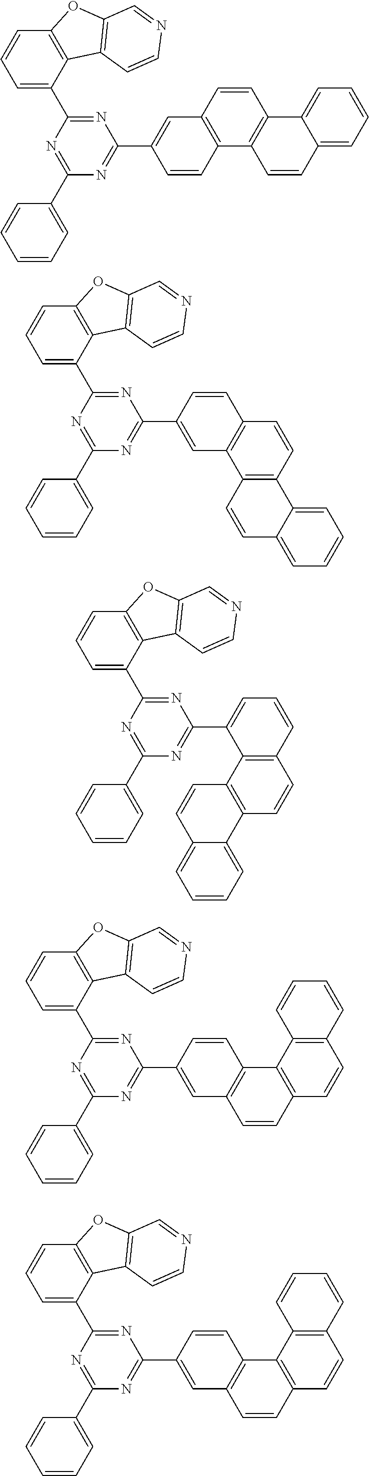

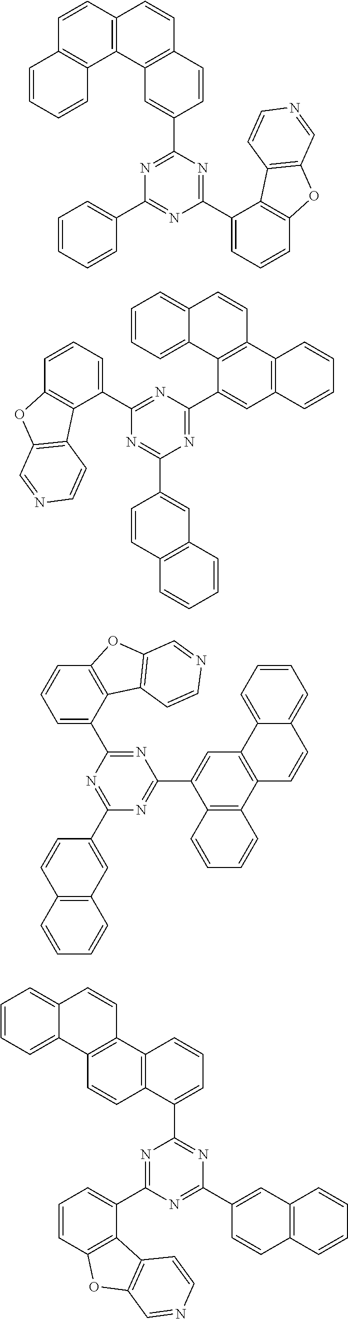

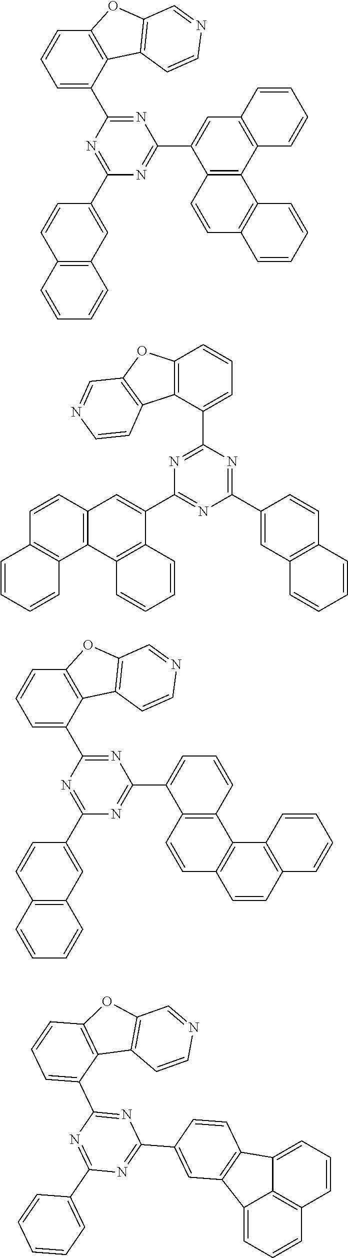

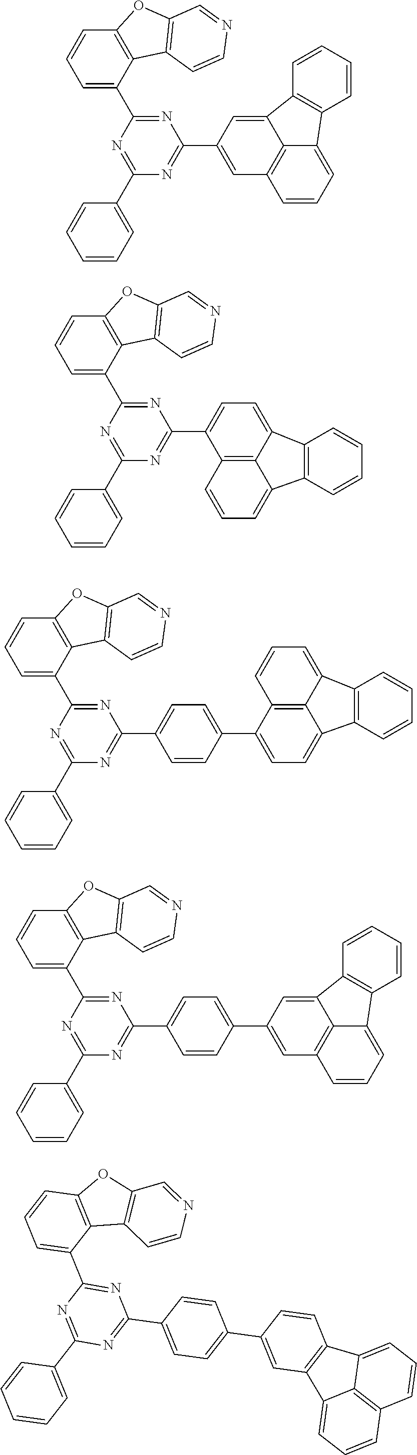

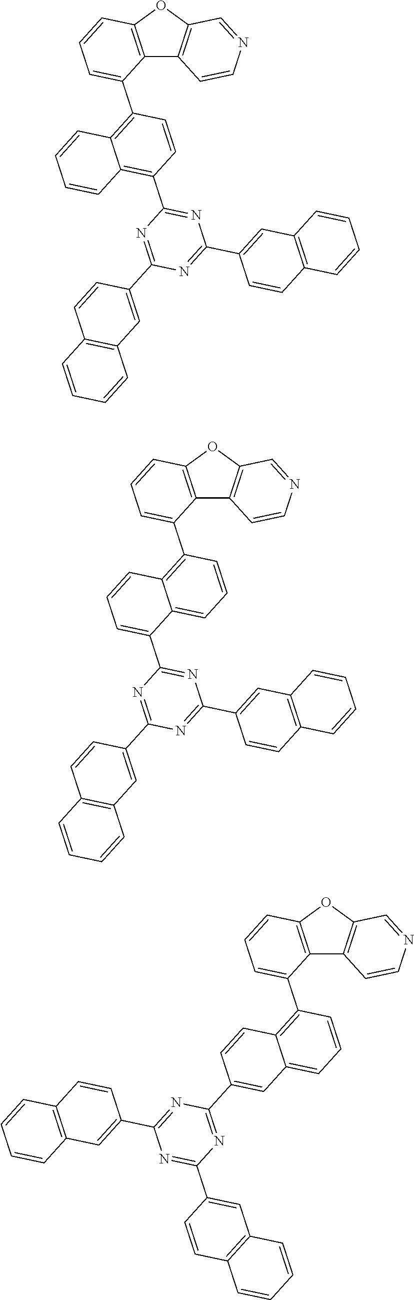 [SMILES: c1ccc2cc(-c3nc(-c4ccc5ccccc5c4)nc(-c4ccc(-c5cccc6oc7cnccc7c56)c5ccccc45)n3)ccc2c1 ; c1ccc2cc(-c3nc(-c4ccc5ccccc5c4)nc(-c4cccc5c(-c6cccc7oc8cnccc8c67)cccc45)n3)ccc2c1 ; c1ccc2cc(-c3nc(-c4ccc5ccccc5c4)nc(-c4ccc5c(-c6cccc7oc8cnccc8c67)cccc5c4)n3)ccc2c1]

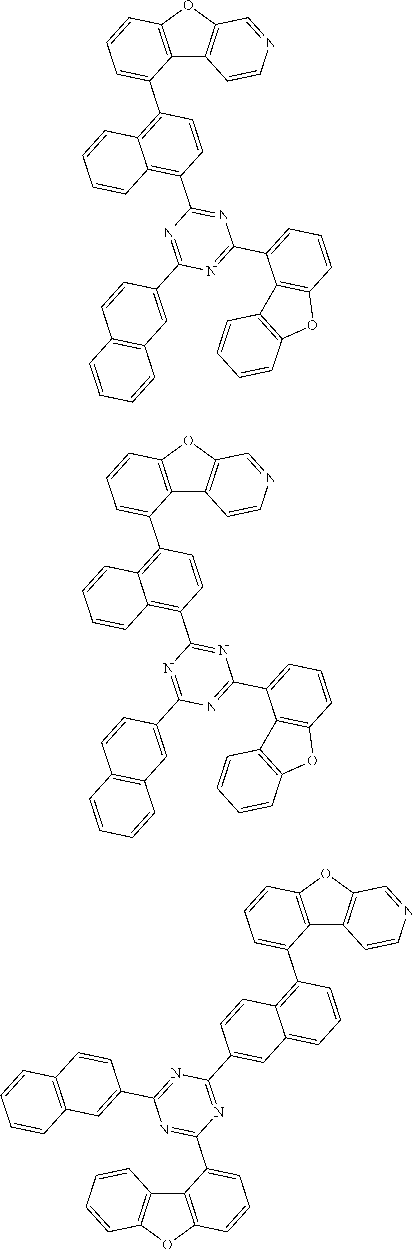 [SMILES: c1ccc2cc(-c3nc(-c4ccc(-c5cccc6oc7cnccc7c56)c5ccccc45)nc(-c4cccc5oc6ccccc6c45)n3)ccc2c1 ; c1ccc2cc(-c3nc(-c4ccc(-c5cccc6oc7cnccc7c56)c5ccccc45)nc(-c4cccc5oc6ccccc6c45)n3)ccc2c1 ; c1ccc2cc(-c3nc(-c4ccc5c(-c6cccc7oc8cnccc8c67)cccc5c4)nc(-c4cccc5oc6ccccc6c45)n3)ccc2c1]

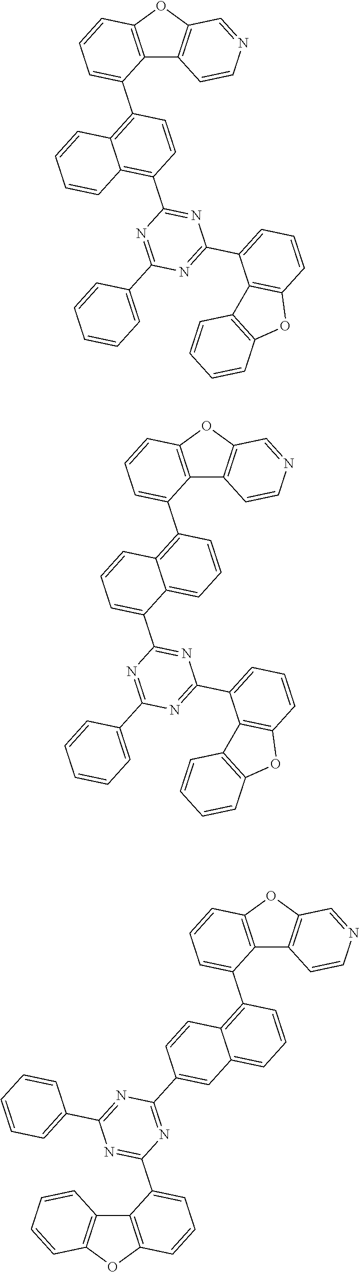 [SMILES: c1ccc(-c2nc(-c3ccc(-c4cccc5oc6cnccc6c45)c4ccccc34)nc(-c3cccc4oc5ccccc5c34)n2)cc1 ; c1ccc(-c2nc(-c3cccc4c(-c5cccc6oc7cnccc7c56)cccc34)nc(-c3cccc4oc5ccccc5c34)n2)cc1 ; c1ccc(-c2nc(-c3ccc4c(-c5cccc6oc7cnccc7c56)cccc4c3)nc(-c3cccc4oc5ccccc5c34)n2)cc1]

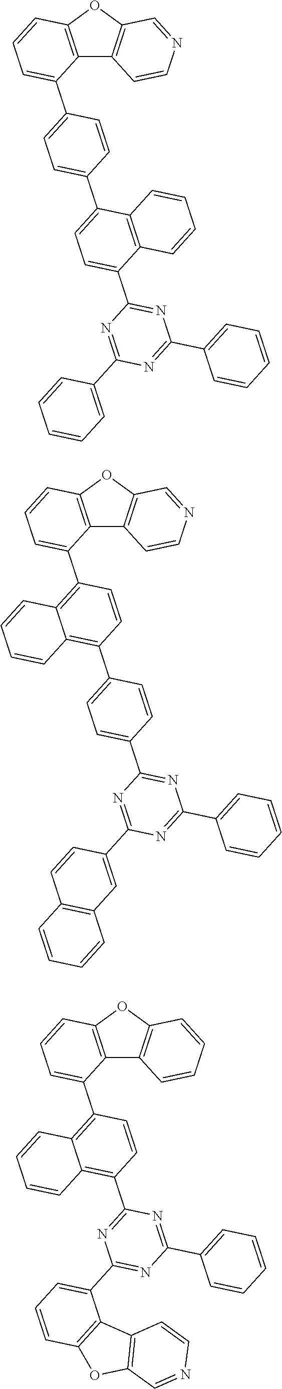

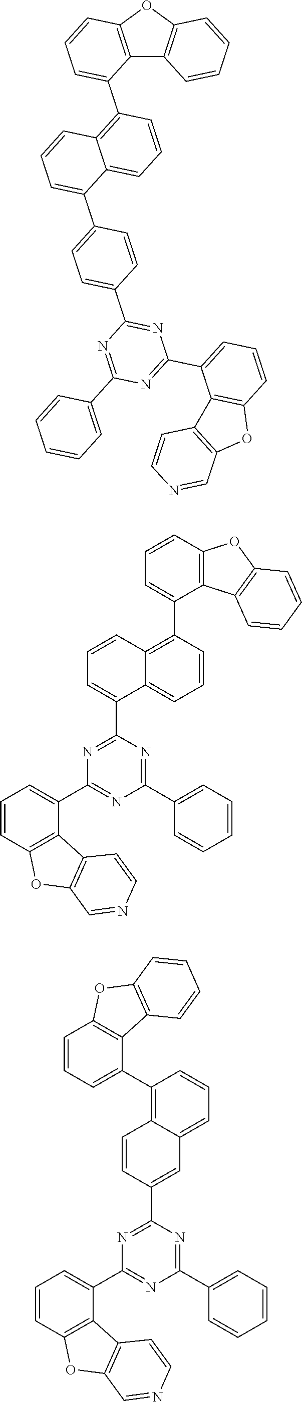

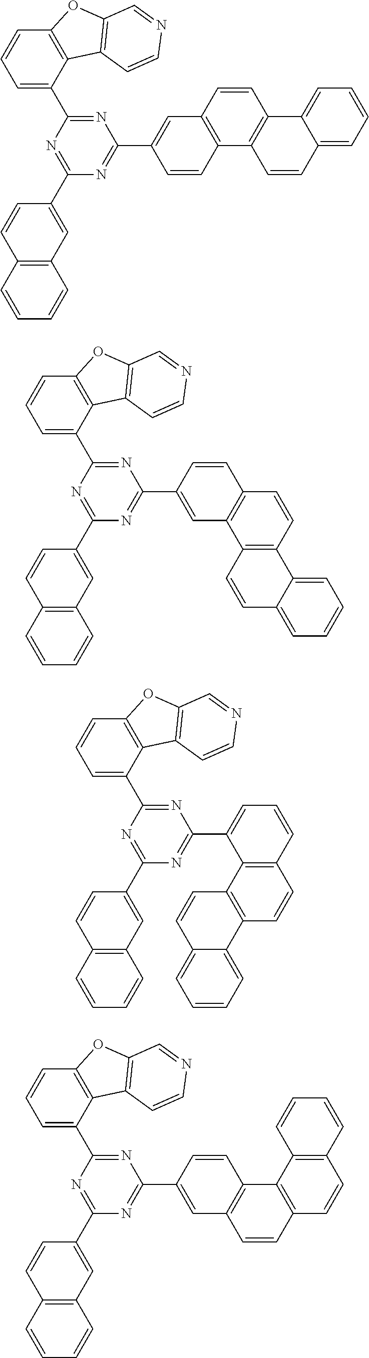

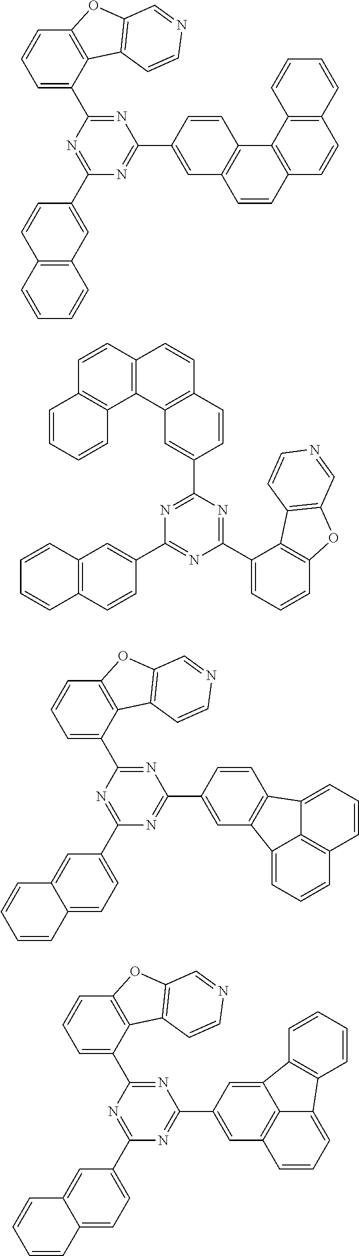

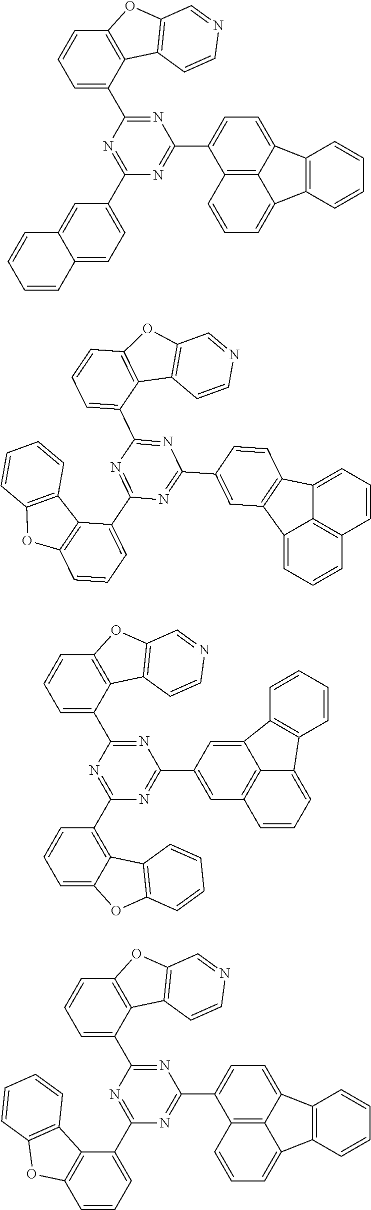 [SMILES: c1ccc2c(c1)-c1cccc3c(-c4nc(-c5ccc6ccccc6c5)nc(-c5cccc6oc7cnccc7c56)n4)ccc-2c13 ; c1cc2c3c(cccc3c1)-c1cc(-c3nc(-c4cccc5oc6ccccc6c45)nc(-c4cccc5oc6cnccc6c45)n3)ccc1-2 ; c1ccc2c(c1)-c1cccc3cc(-c4nc(-c5cccc6oc7ccccc7c56)nc(-c5cccc6oc7cnccc7c56)n4)cc-2c13 ; c1ccc2c(c1)-c1cccc3c(-c4nc(-c5cccc6oc7ccccc7c56)nc(-c5cccc6oc7cnccc7c56)n4)ccc-2c13]

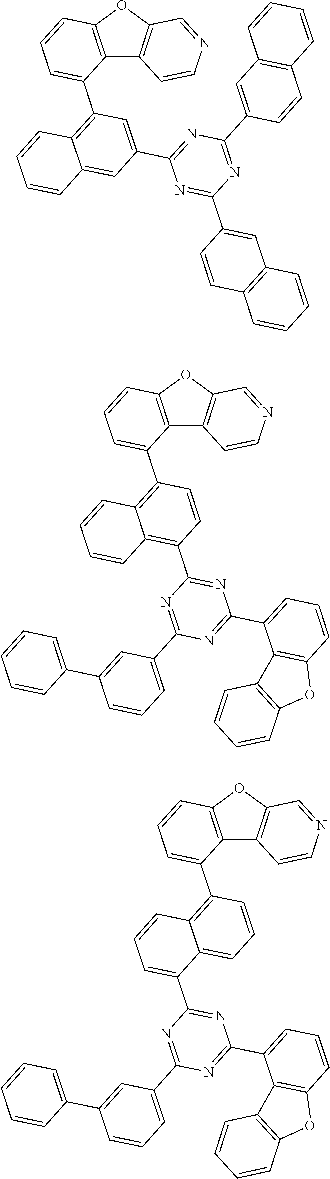

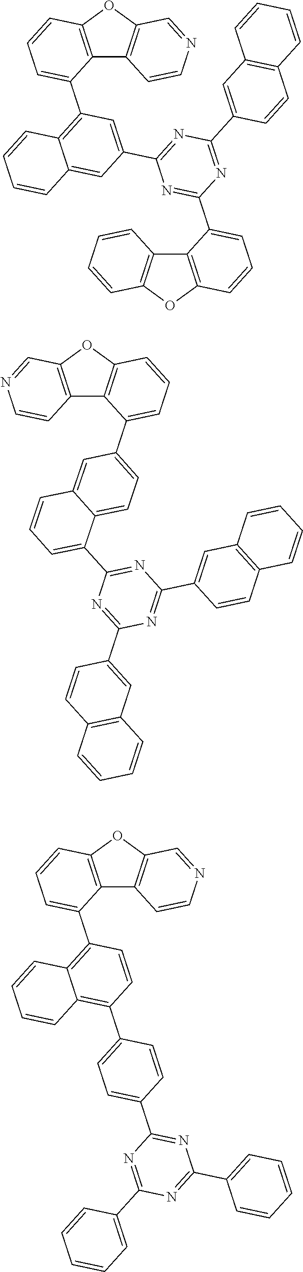 [SMILES: c1ccc2cc(-c3nc(-c4cc(-c5cccc6oc7cnccc7c56)c5ccccc5c4)nc(-c4cccc5oc6ccccc6c45)n3)ccc2c1 ; c1ccc2cc(-c3nc(-c4ccc5ccccc5c4)nc(-c4cccc5cc(-c6cccc7oc8cnccc8c67)ccc45)n3)ccc2c1 ; c1ccc(-c2nc(-c3ccccc3)nc(-c3ccc(-c4ccc(-c5cccc6oc7cnccc7c56)c5ccccc45)cc3)n2)cc1]

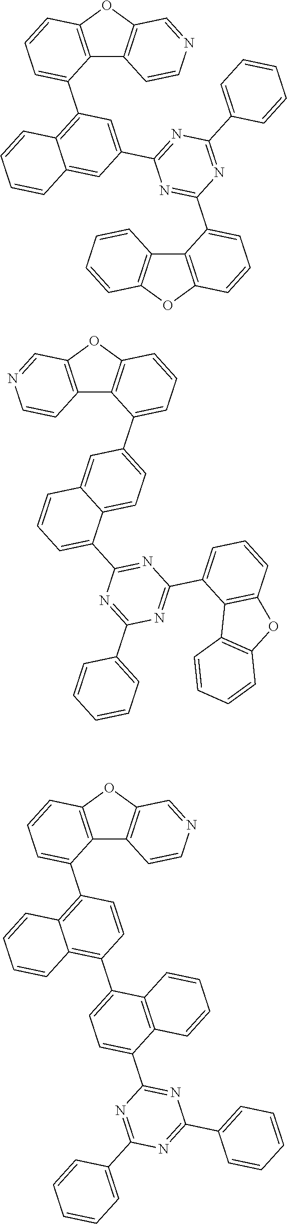 [SMILES: c1ccc(-c2nc(-c3cc(-c4cccc5oc6cnccc6c45)c4ccccc4c3)nc(-c3cccc4oc5ccccc5c34)n2)cc1 ; c1ccc(-c2nc(-c3cccc4cc(-c5cccc6oc7cnccc7c56)ccc34)nc(-c3cccc4oc5ccccc5c34)n2)cc1 ; c1ccc(-c2nc(-c3ccccc3)nc(-c3ccc(-c4ccc(-c5cccc6oc7cnccc7c56)c5ccccc45)c4ccccc34)n2)cc1]

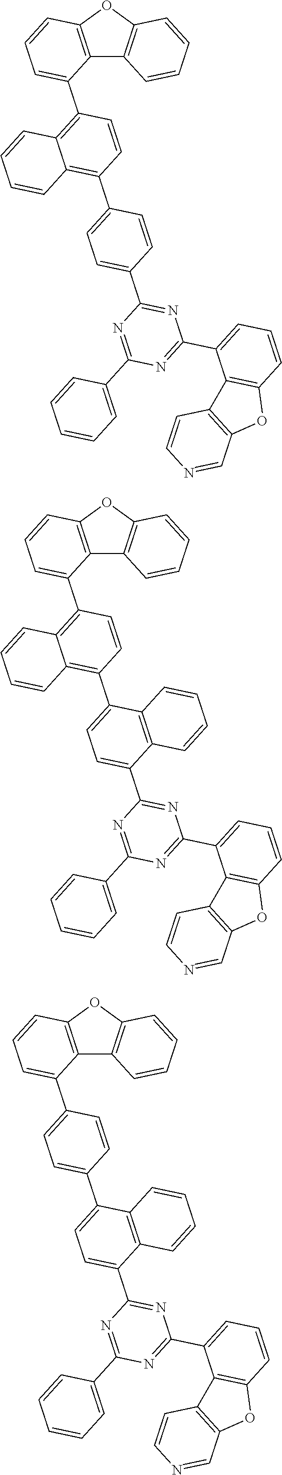 [SMILES: c1ccc(-c2nc(-c3ccc(-c4ccc(-c5cccc6oc7ccccc7c56)c5ccccc45)cc3)nc(-c3cccc4oc5cnccc5c34)n2)cc1 ; c1ccc(-c2nc(-c3ccc(-c4ccc(-c5cccc6oc7ccccc7c56)c5ccccc45)c4ccccc34)nc(-c3cccc4oc5cnccc5c34)n2)cc1 ; c1ccc(-c2nc(-c3ccc(-c4ccc(-c5cccc6oc7ccccc7c56)cc4)c4ccccc34)nc(-c3cccc4oc5cnccc5c34)n2)cc1]

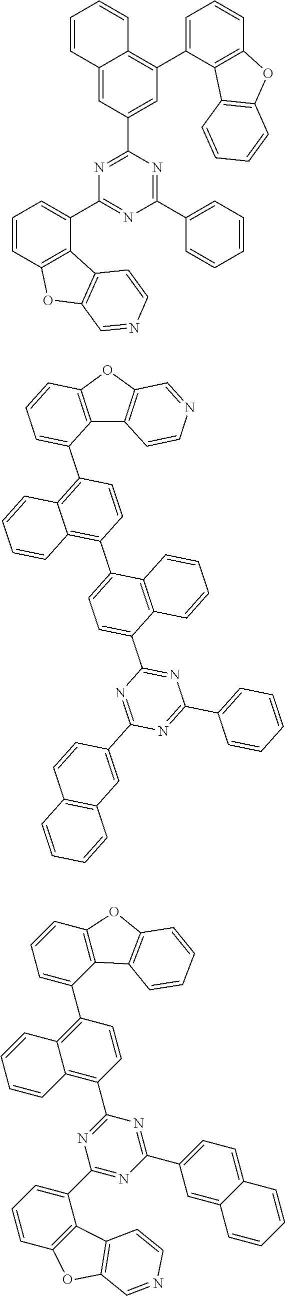

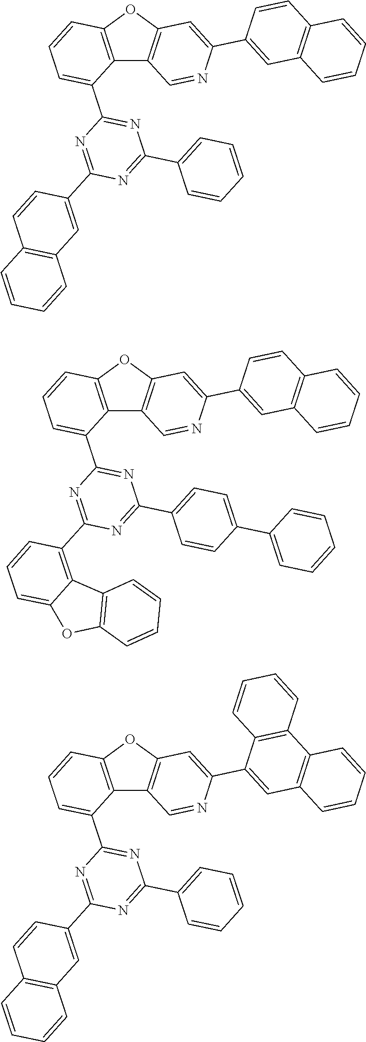 [SMILES: c1ccc(-c2nc(-c3ccc4ccccc4c3)nc(-c3cccc4oc5cc(-c6ccc7ccccc7c6)ncc5c34)n2)cc1 ; c1ccc(-c2ccc(-c3nc(-c4cccc5oc6ccccc6c45)nc(-c4cccc5oc6cc(-c7ccc8ccccc8c7)ncc6c45)n3)cc2)cc1 ; c1ccc(-c2nc(-c3ccc4ccccc4c3)nc(-c3cccc4oc5cc(-c6cc7ccccc7c7ccccc67)ncc5c34)n2)cc1]

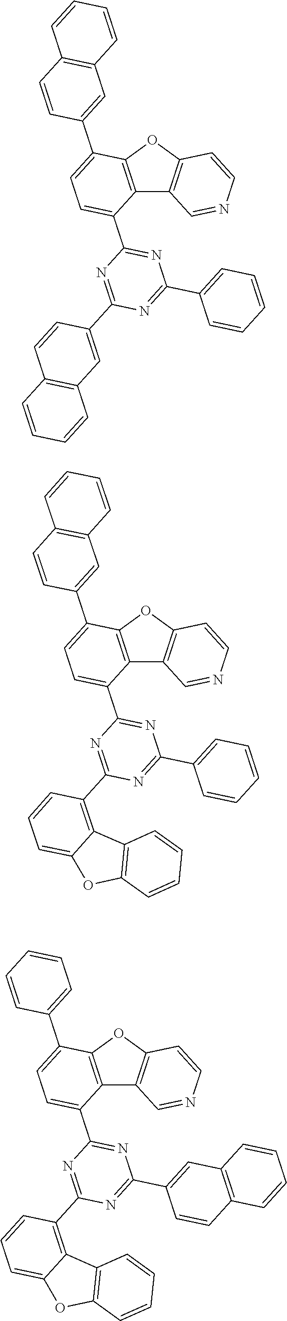

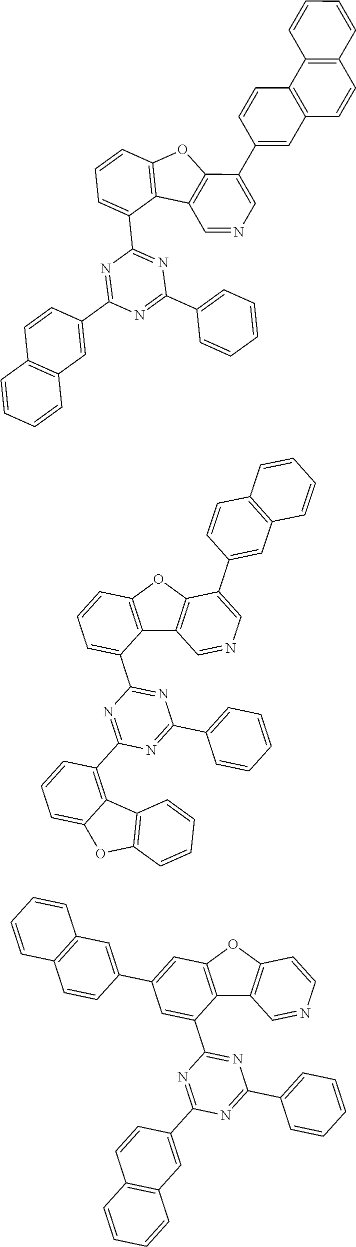 [SMILES: c1ccc(-c2nc(-c3ccc4ccccc4c3)nc(-c3cccc4oc5c(-c6ccc7c(ccc8ccccc87)c6)cncc5c34)n2)cc1 ; c1ccc(-c2nc(-c3cccc4oc5ccccc5c34)nc(-c3cccc4oc5c(-c6ccc7ccccc7c6)cncc5c34)n2)cc1 ; c1ccc(-c2nc(-c3ccc4ccccc4c3)nc(-c3cc(-c4ccc5ccccc5c4)cc4oc5ccncc5c34)n2)cc1]

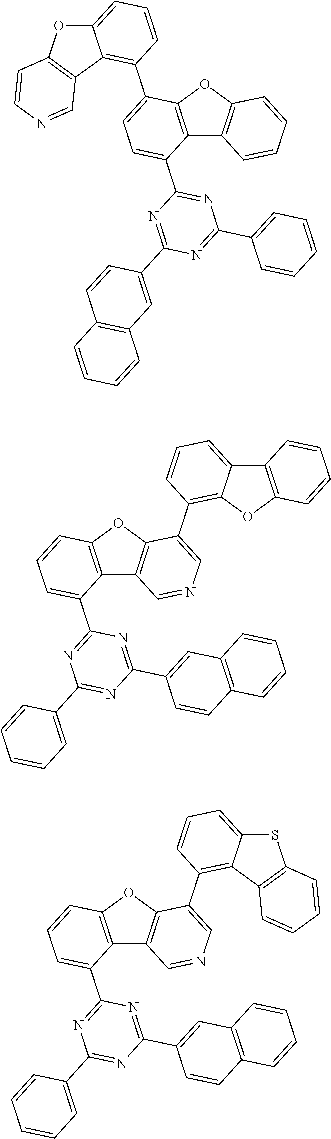 [SMILES: c1ccc(-c2nc(-c3ccc4ccccc4c3)nc(-c3ccc(-c4cccc5oc6ccncc6c45)c4oc5ccccc5c34)n2)cc1 ; c1ccc(-c2nc(-c3ccc4ccccc4c3)nc(-c3cccc4oc5c(-c6cccc7c6oc6ccccc67)cncc5c34)n2)cc1 ; c1ccc(-c2nc(-c3ccc4ccccc4c3)nc(-c3cccc4oc5c(-c6cccc7sc8ccccc8c67)cncc5c34)n2)cc1]

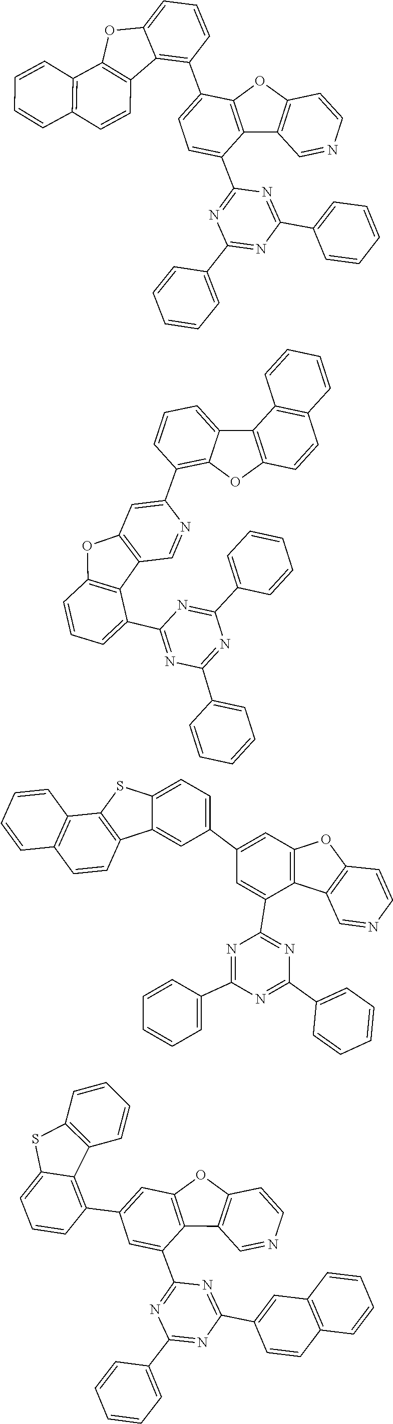

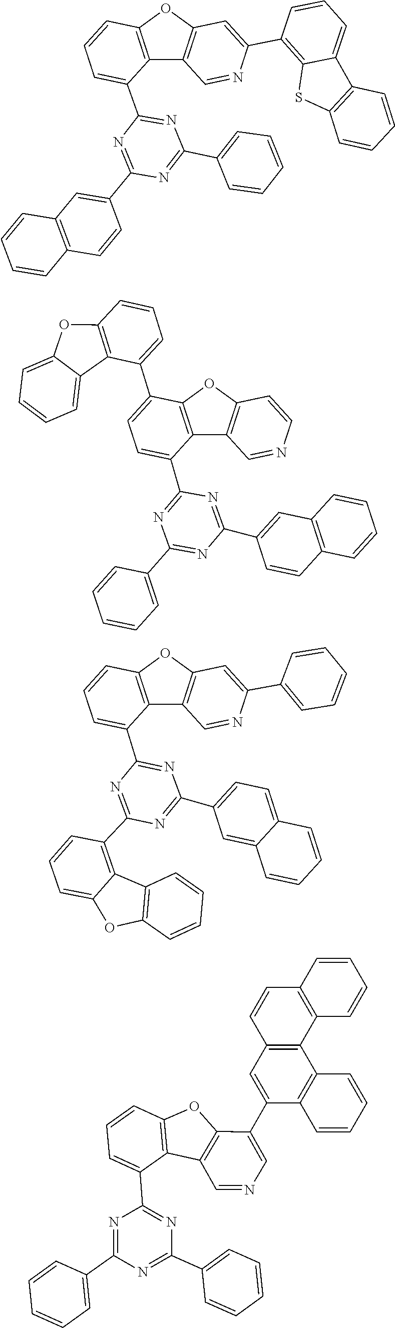

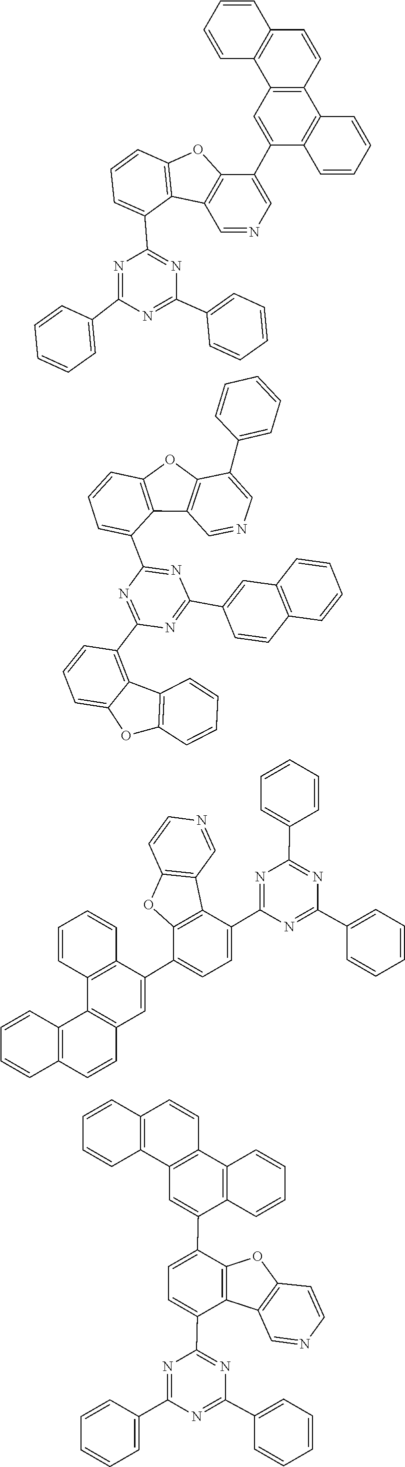

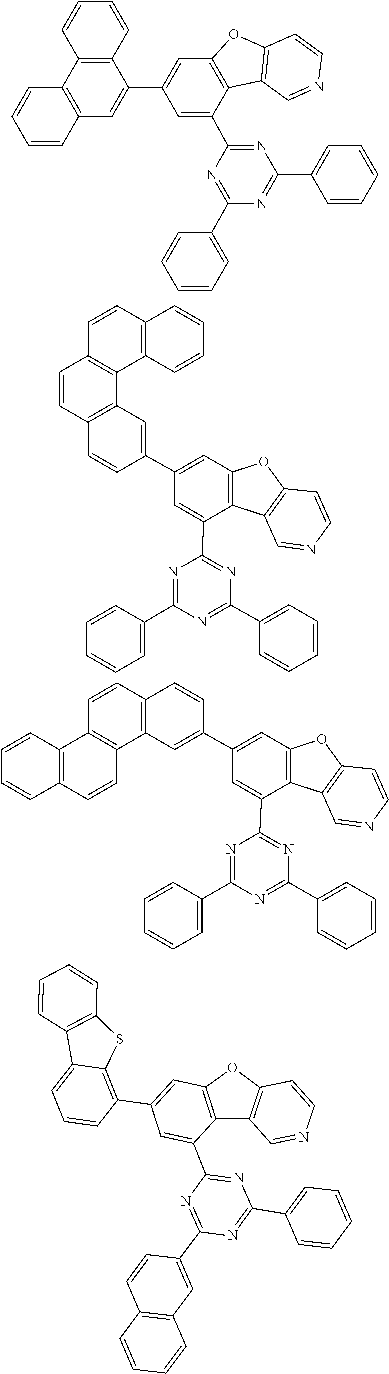

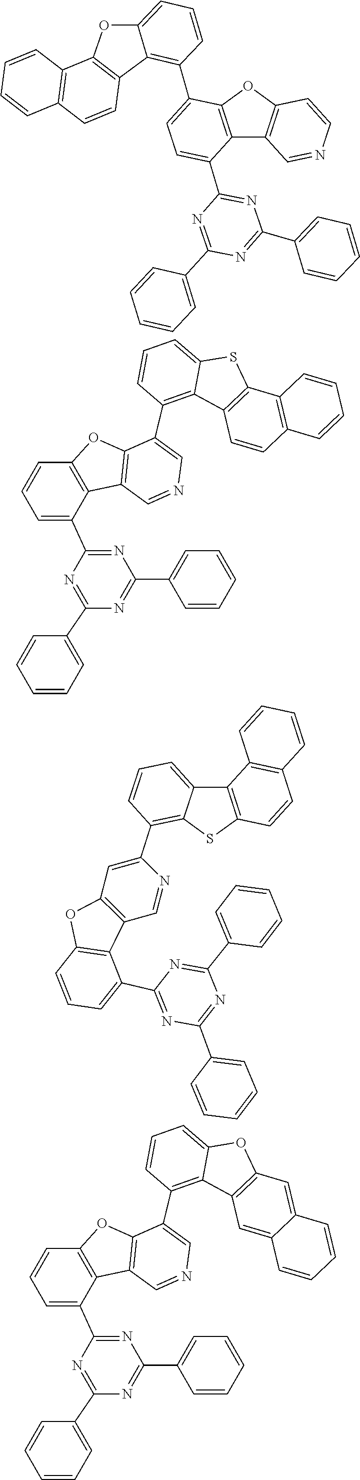

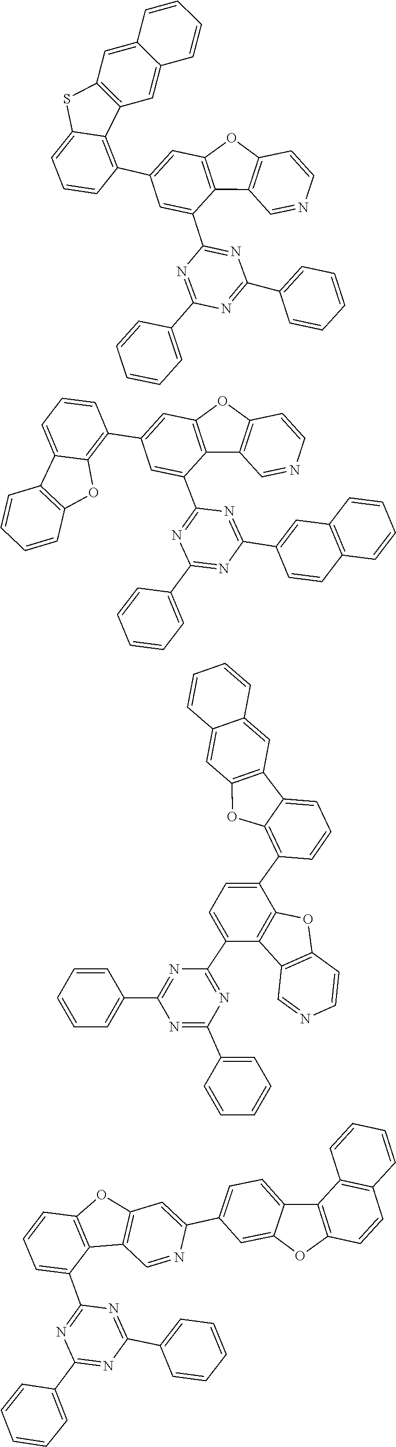

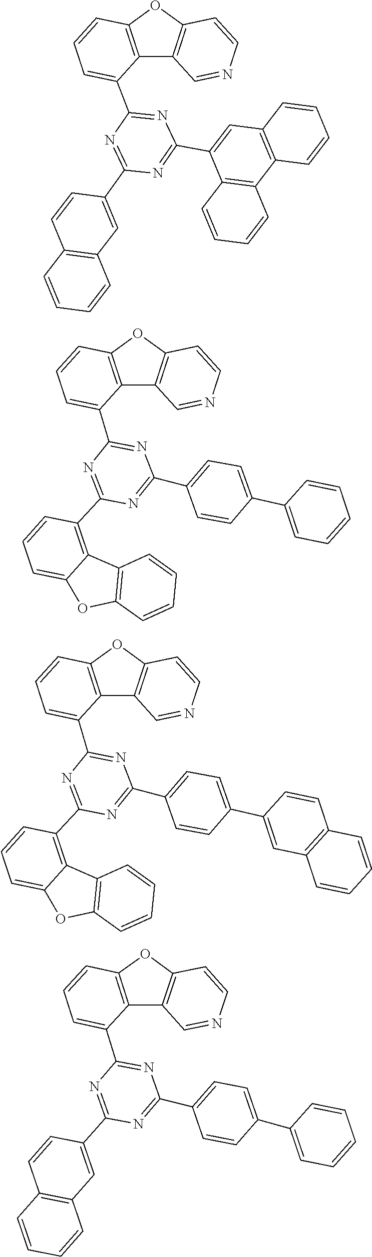

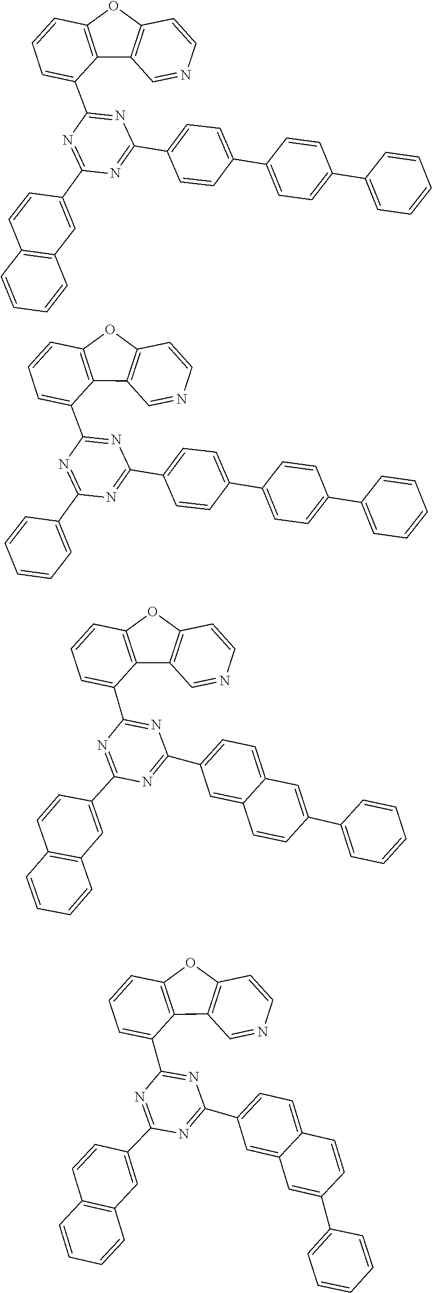

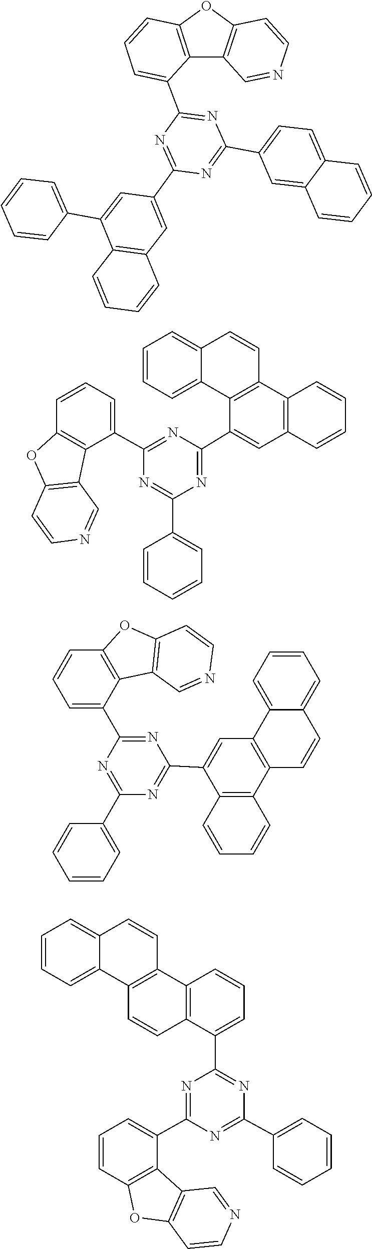

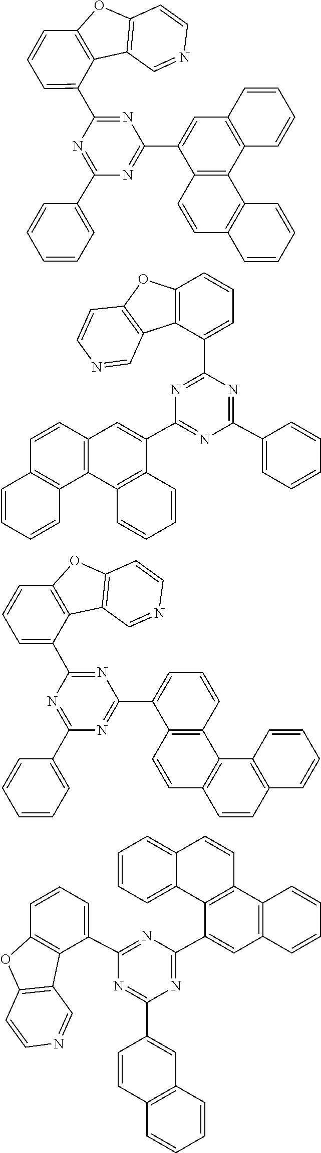

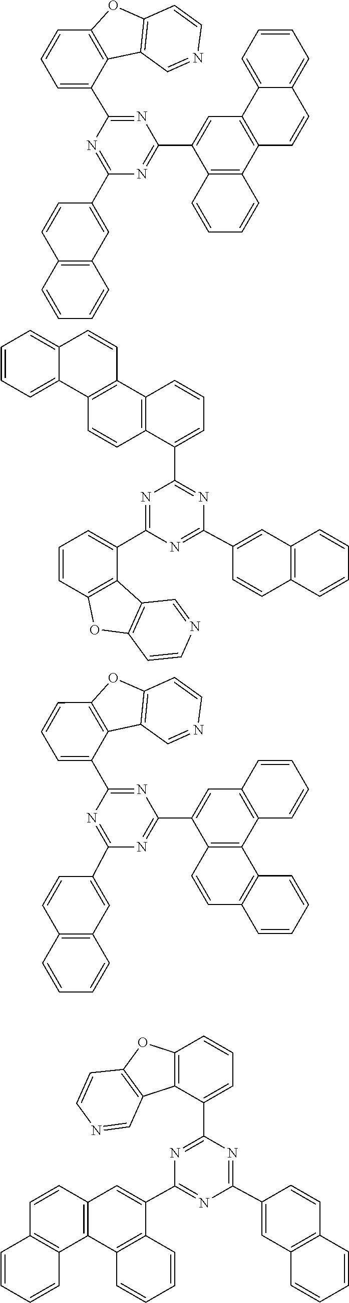

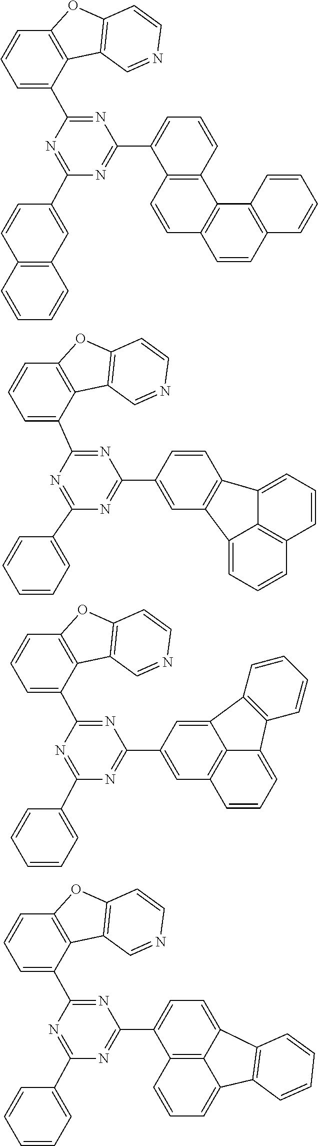

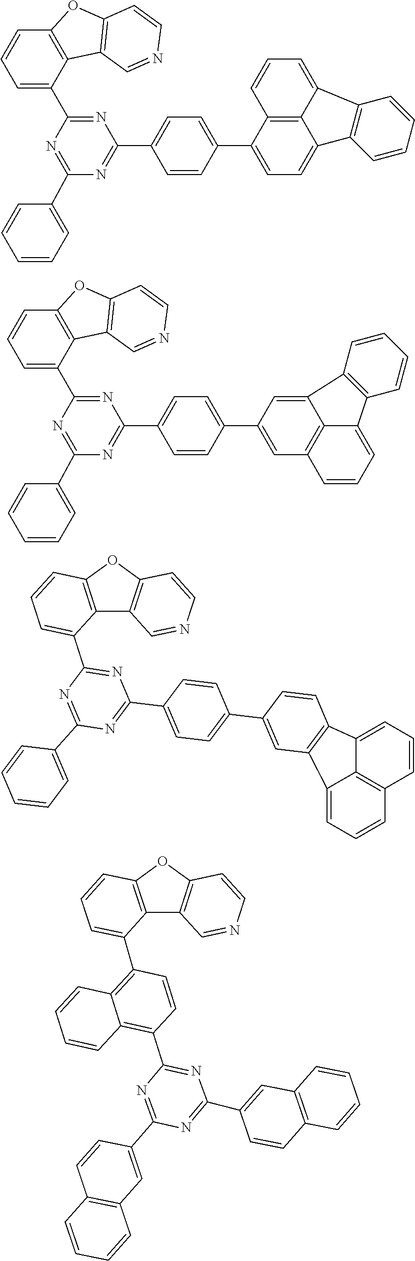

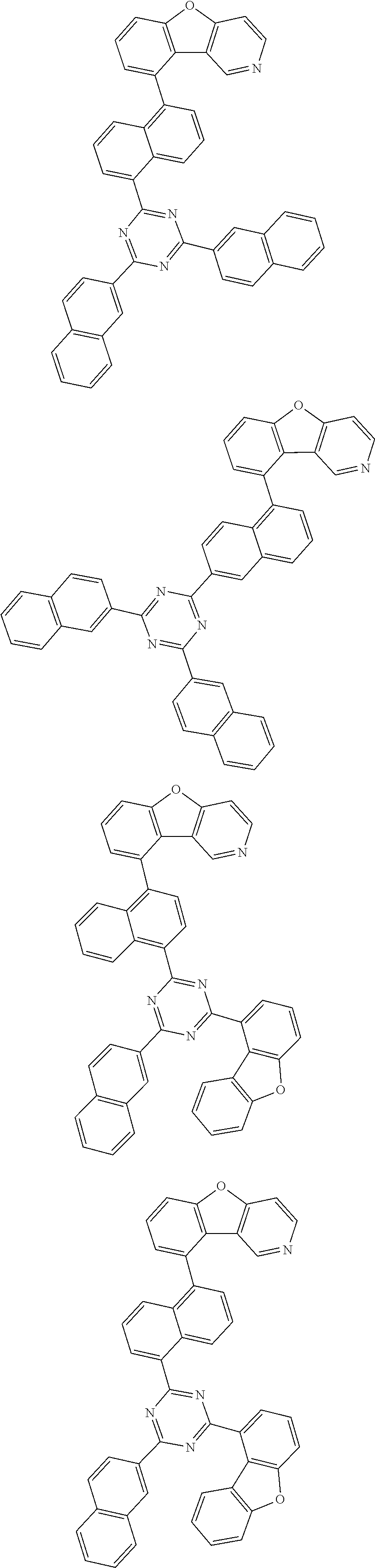

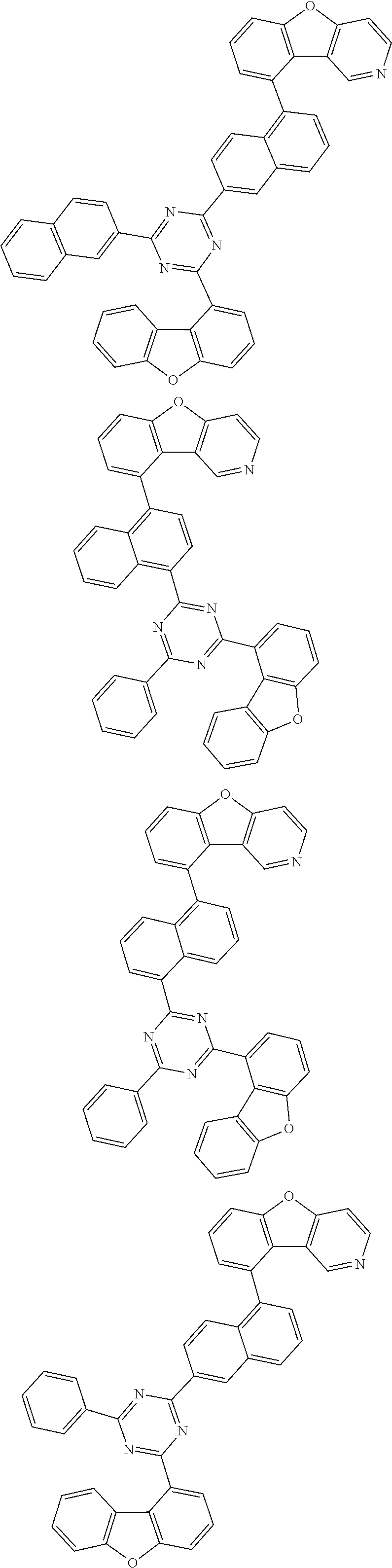

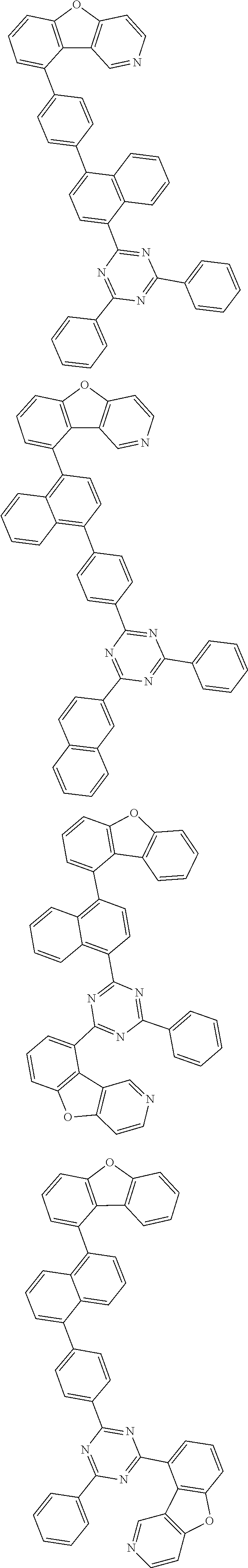

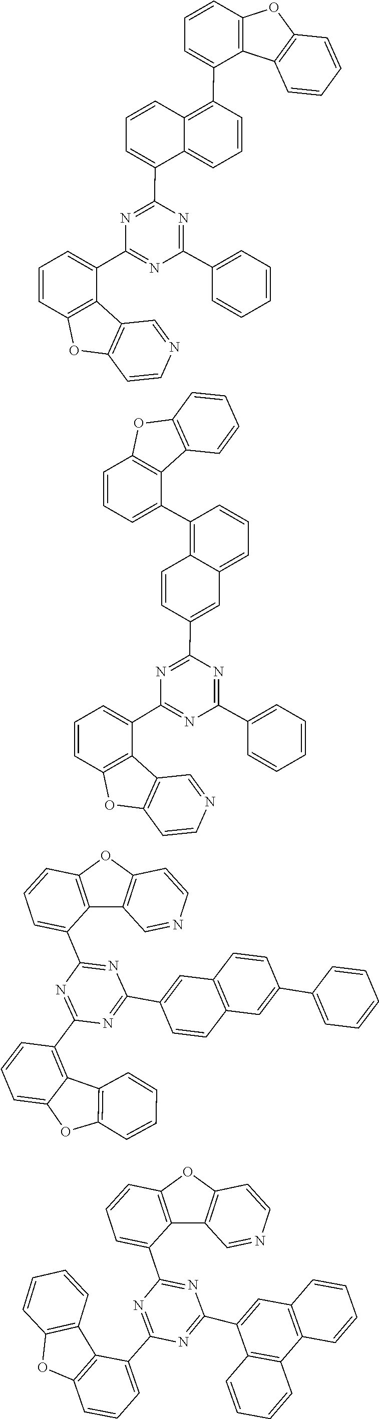

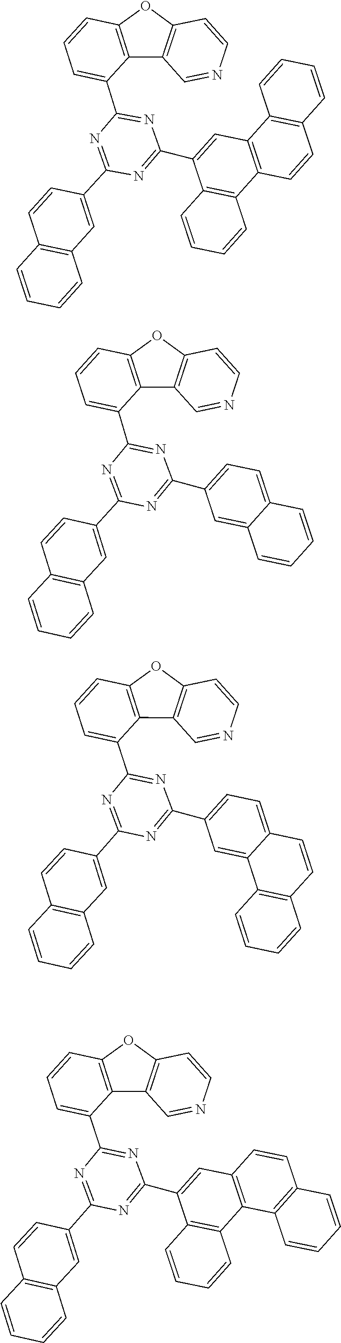 [SMILES: c1ccc2cc(-c3nc(-c4cc5c6ccccc6ccc5c5ccccc45)nc(-c4cccc5oc6ccncc6c45)n3)ccc2c1 ; c1ccc2cc(-c3nc(-c4ccc5ccccc5c4)nc(-c4cccc5oc6ccncc6c45)n3)ccc2c1 ; c1ccc2cc(-c3nc(-c4ccc5ccc6ccccc6c5c4)nc(-c4cccc5oc6ccncc6c45)n3)ccc2c1 ; c1ccc2cc(-c3nc(-c4cc5ccc6ccccc6c5c5ccccc45)nc(-c4cccc5oc6ccncc6c45)n3)ccc2c1]

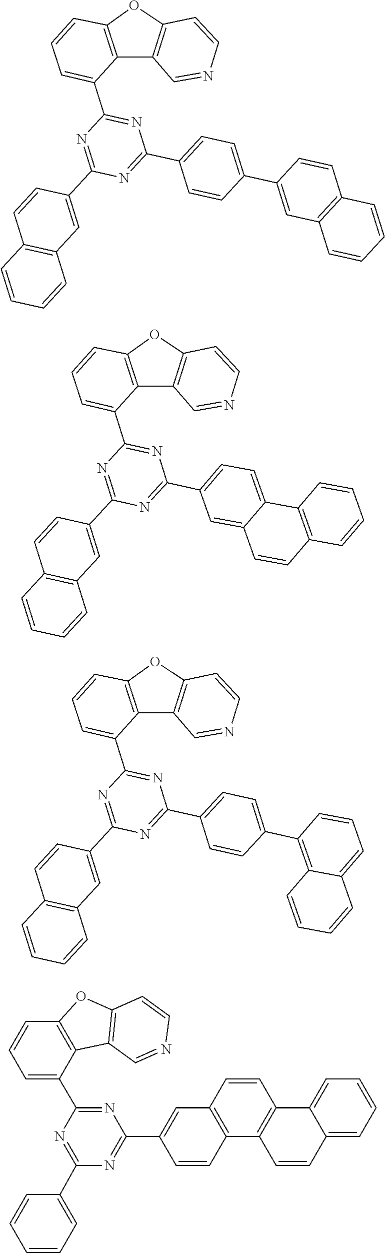

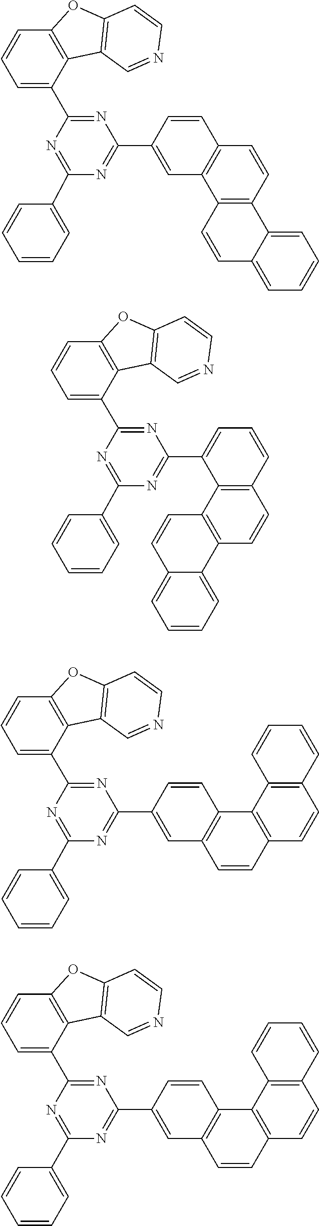 [SMILES: c1ccc(-c2nc(-c3ccc4ccc5c6ccccc6ccc5c4c3)nc(-c3cccc4oc5ccncc5c34)n2)cc1 ; c1ccc(-c2nc(-c3cccc4ccc5c6ccccc6ccc5c34)nc(-c3cccc4oc5ccncc5c34)n2)cc1 ; c1ccc(-c2nc(-c3ccc4c(ccc5ccc6ccccc6c54)c3)nc(-c3cccc4oc5ccncc5c34)n2)cc1 ; c1ccc(-c2nc(-c3ccc4c(ccc5ccc6ccccc6c54)c3)nc(-c3cccc4oc5ccncc5c34)n2)cc1]

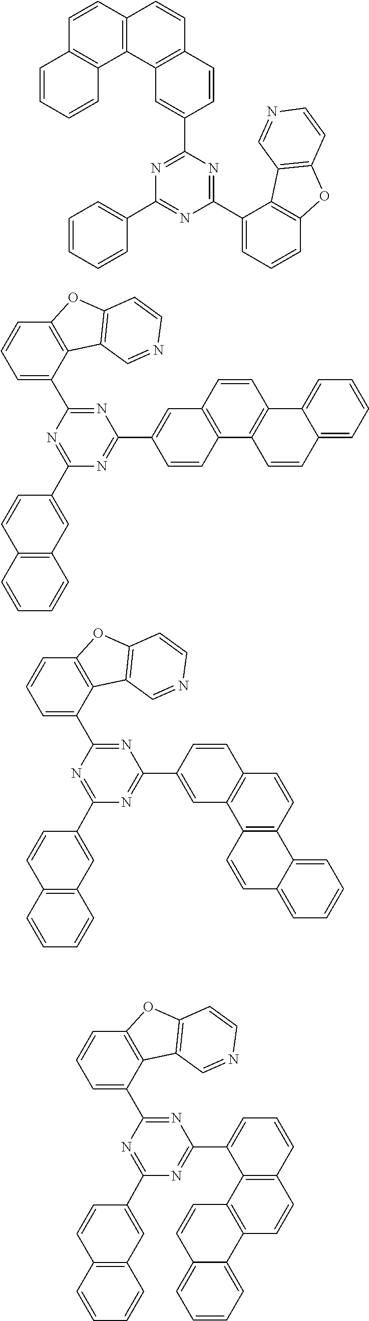

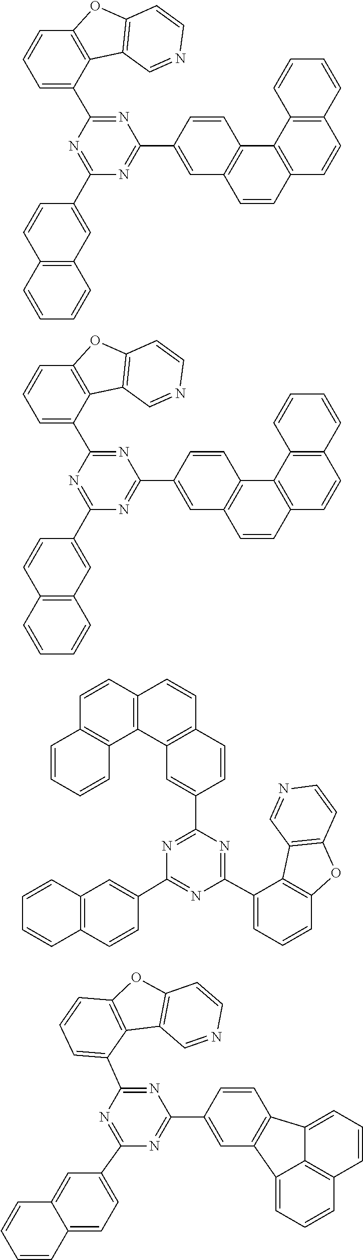

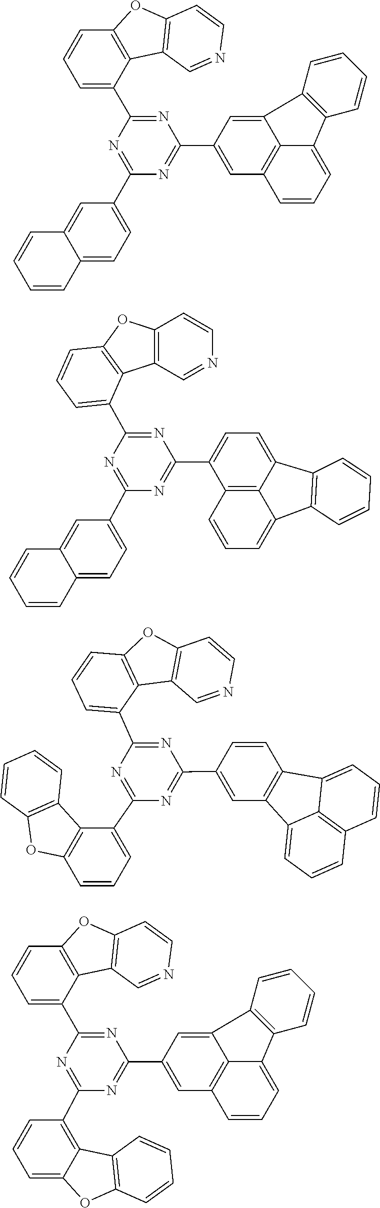

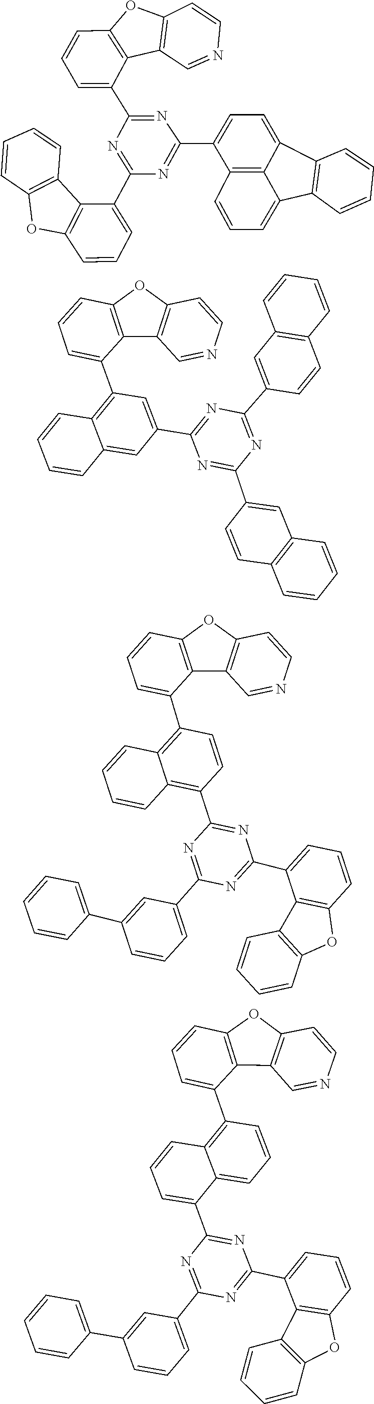

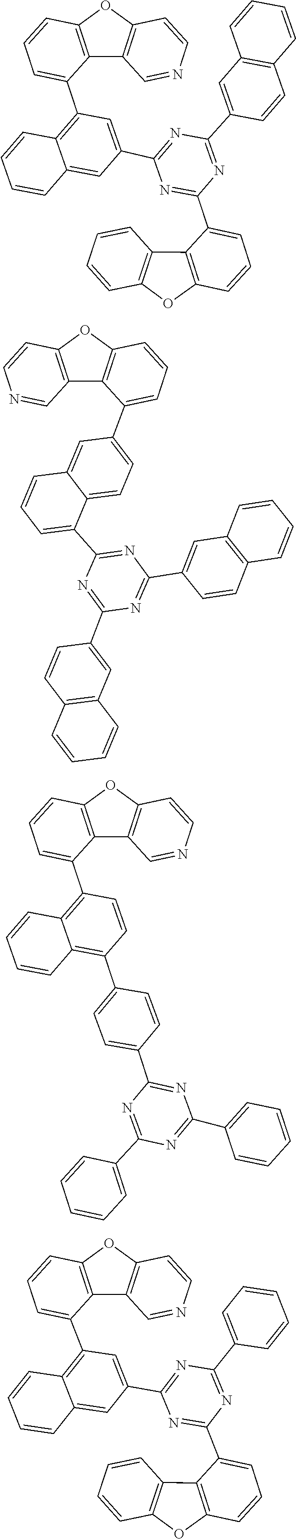

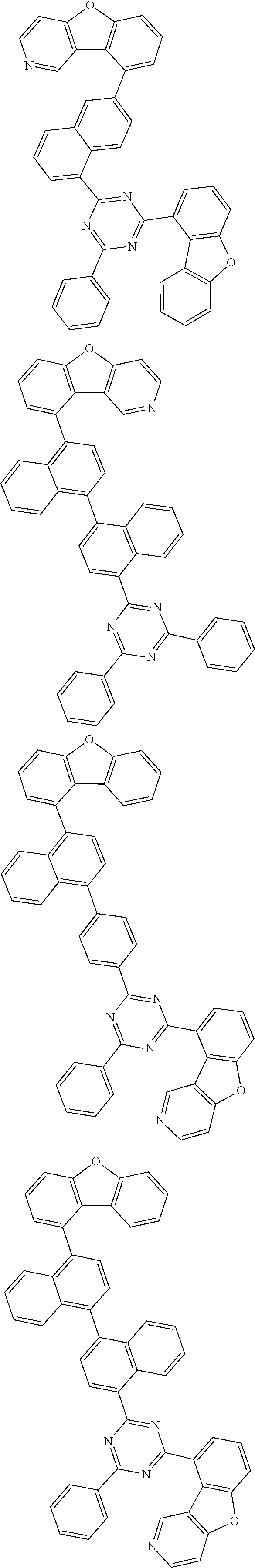

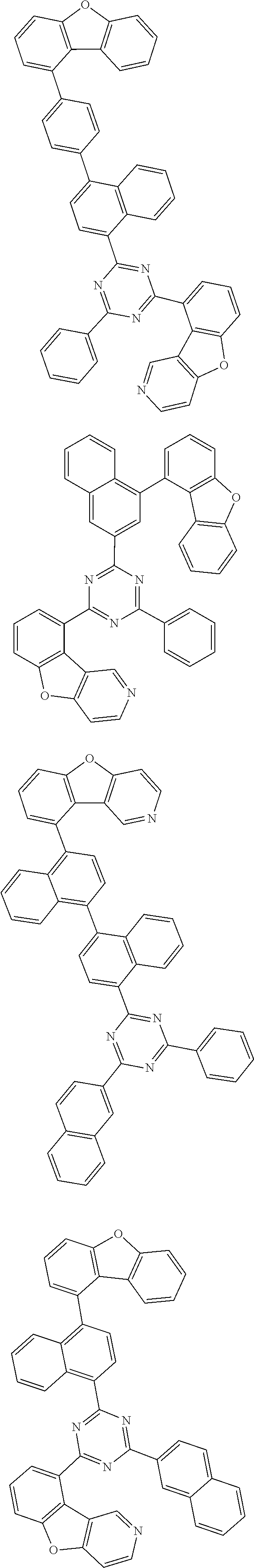

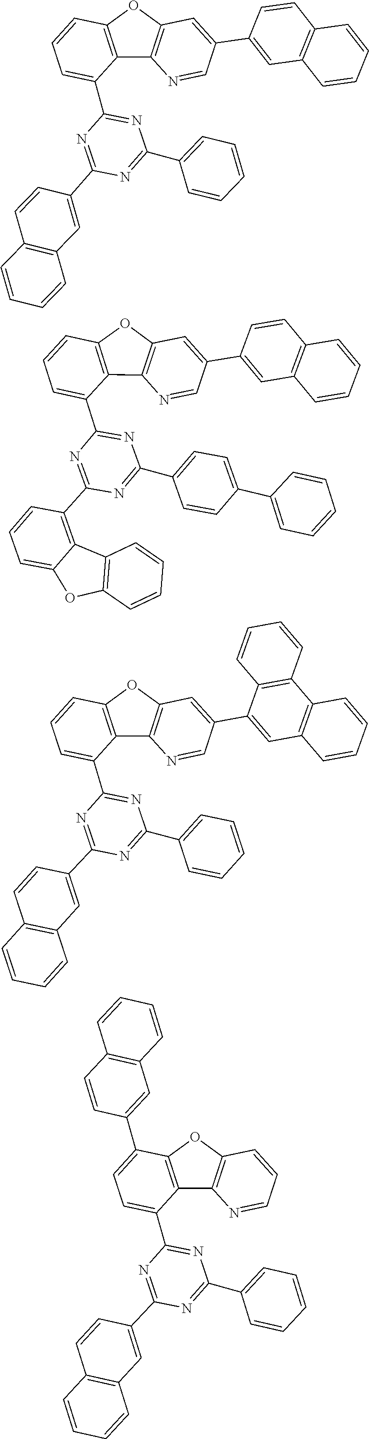

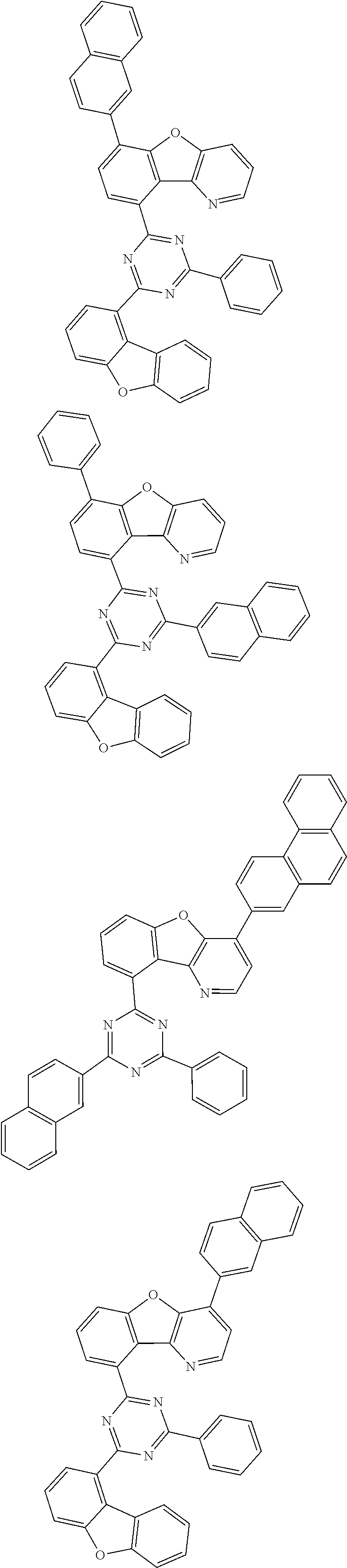

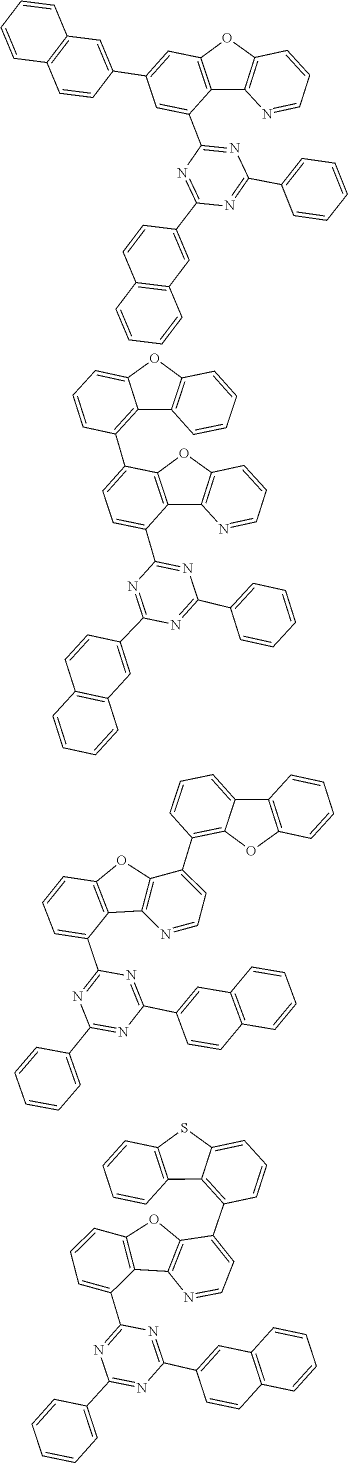

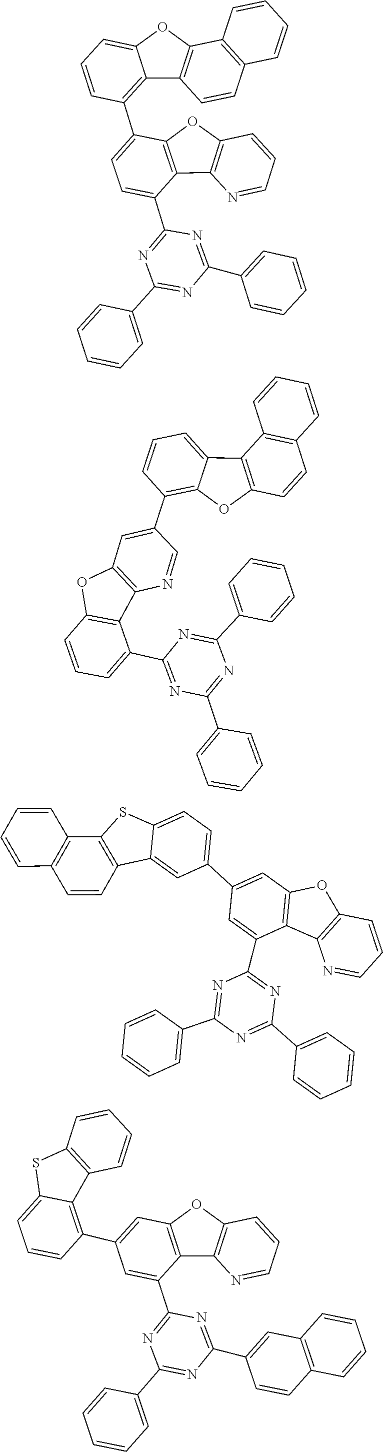

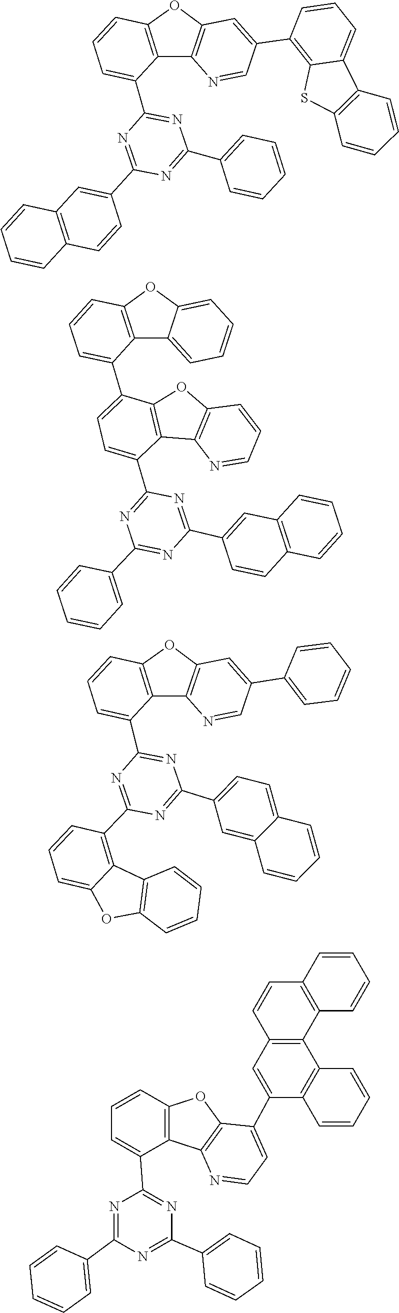

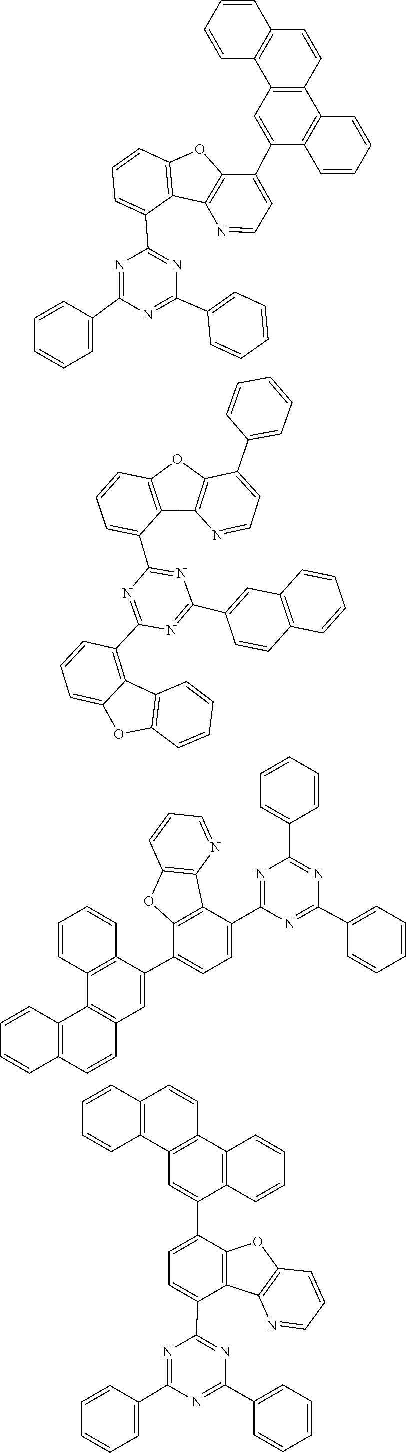

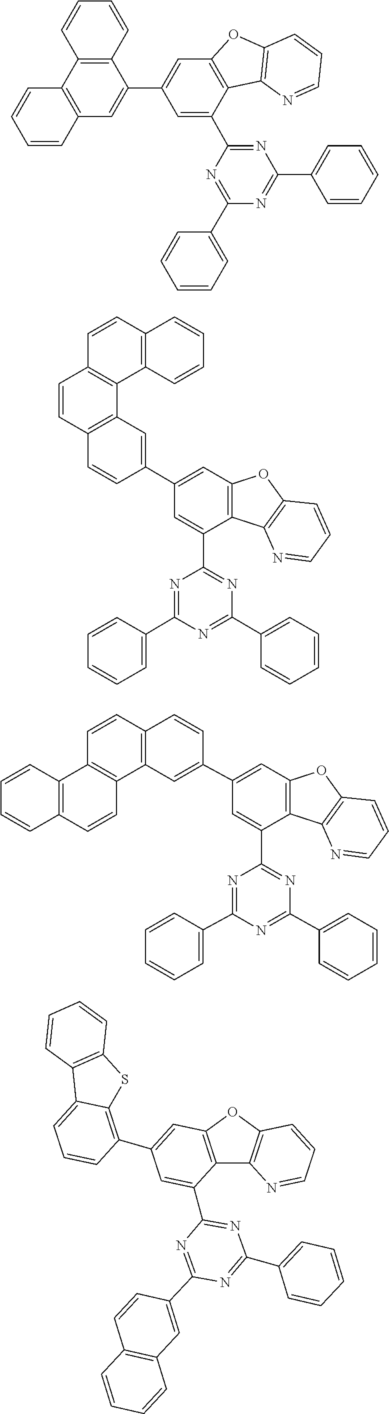

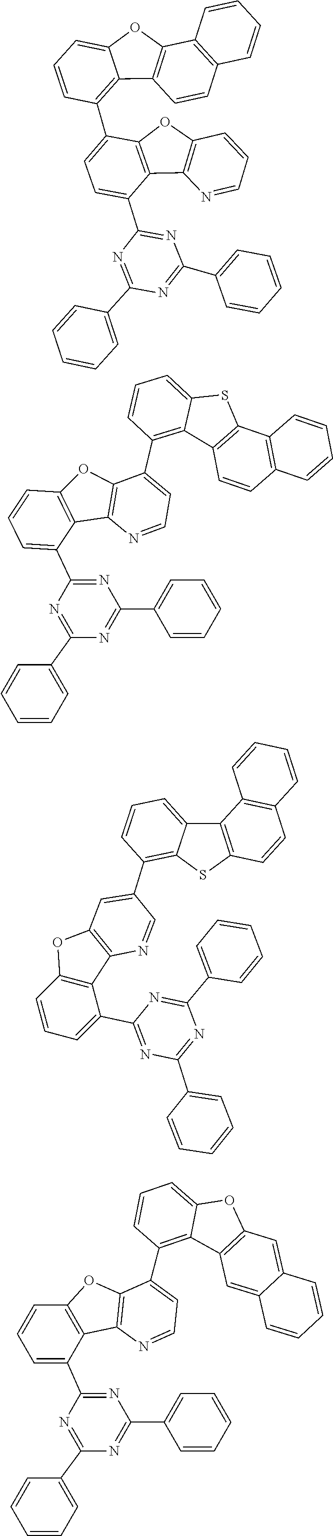

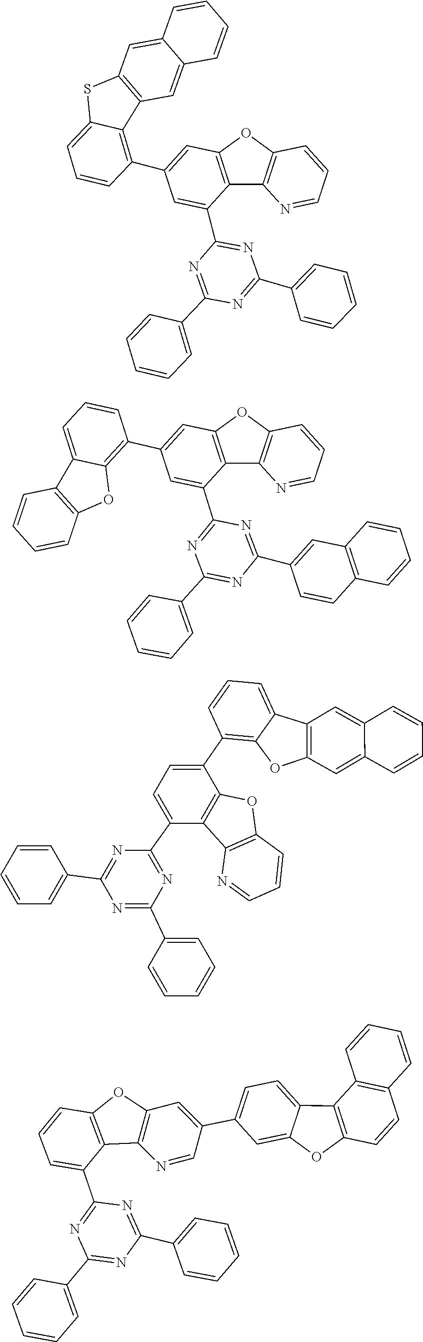

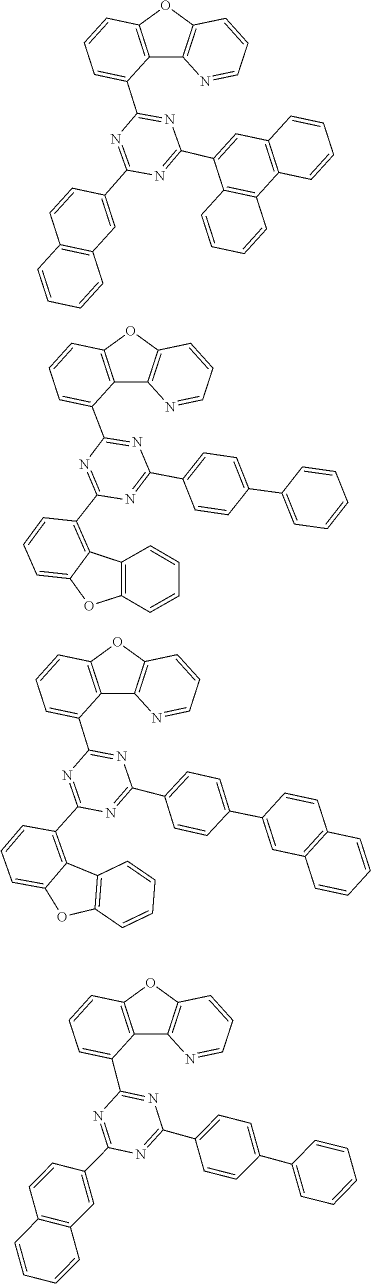

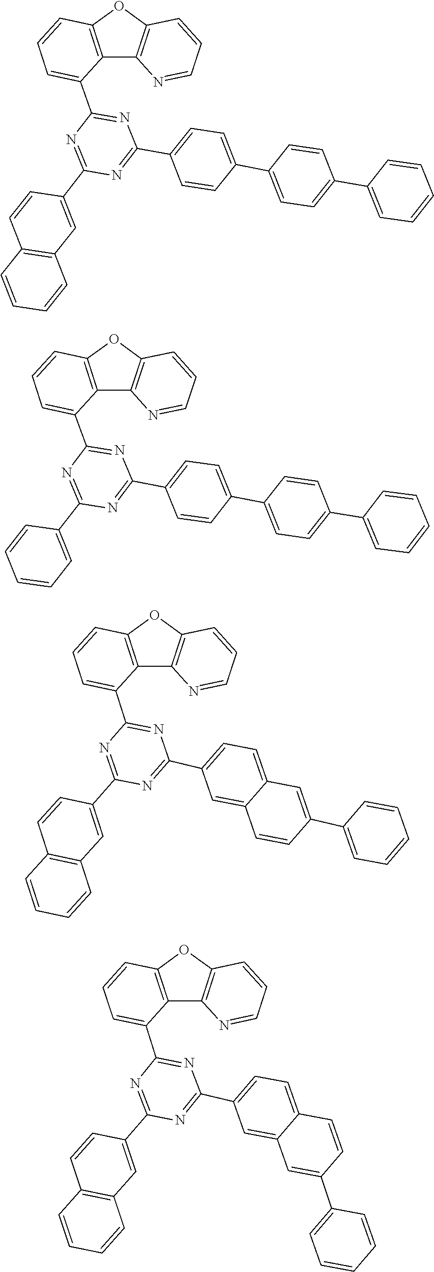

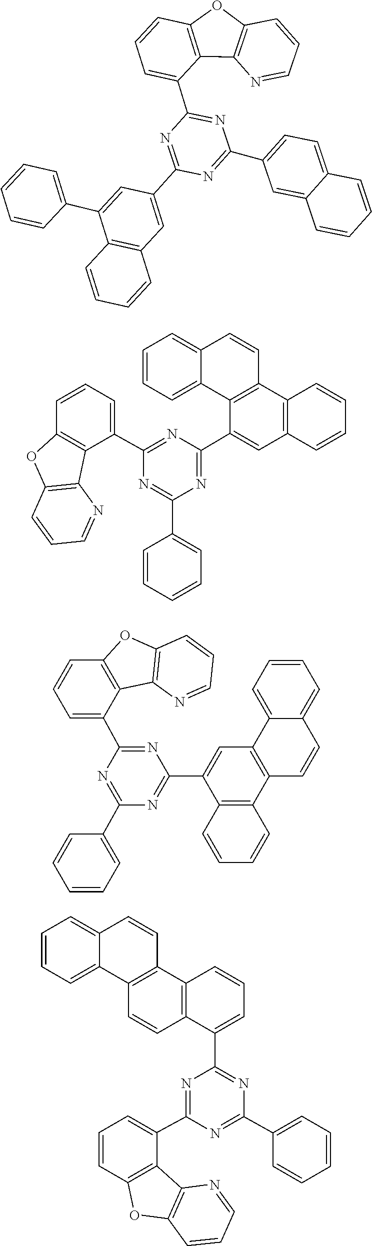

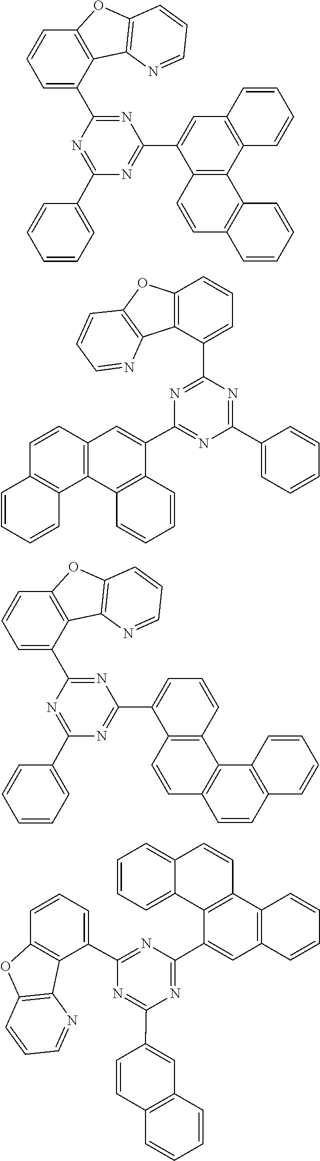

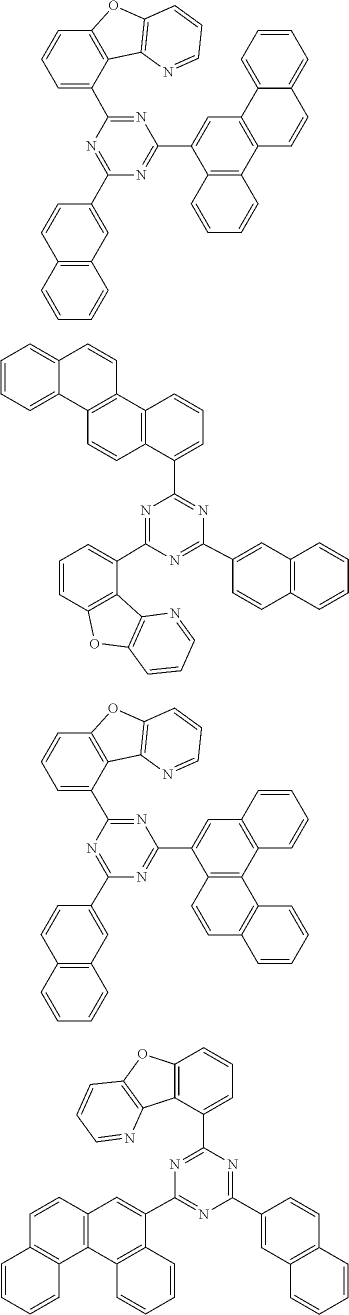

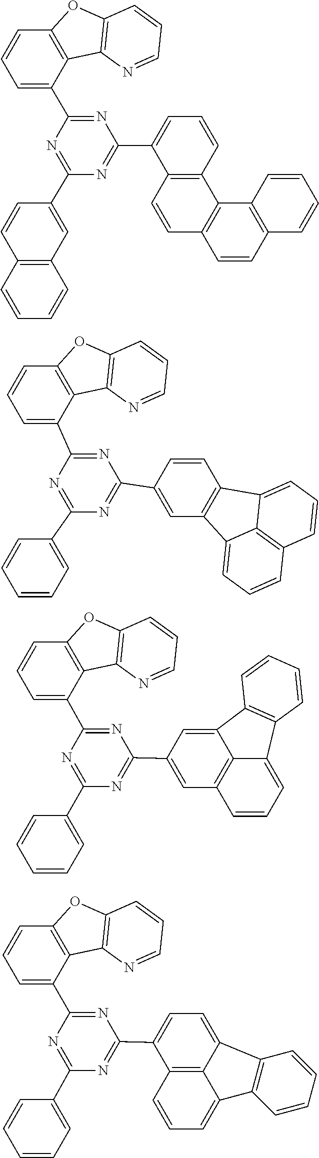

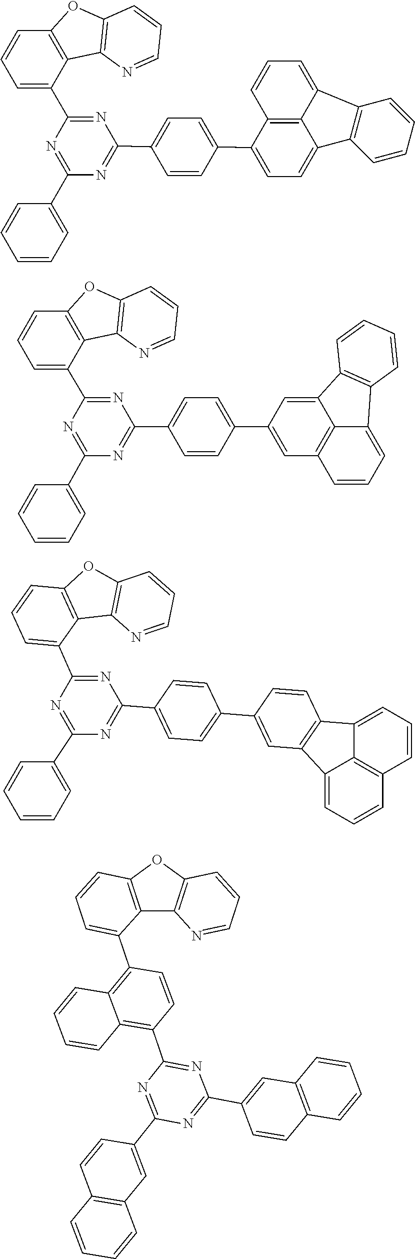

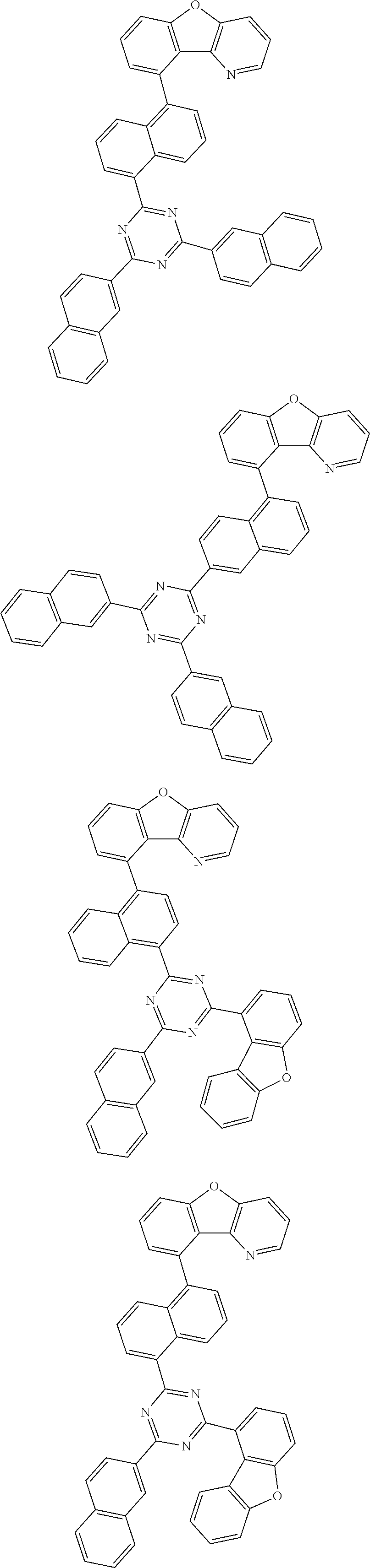 [SMILES: c1ccc2cc(-c3nc(-c4ccc5ccccc5c4)nc(-c4cccc5c(-c6cccc7oc8cccnc8c67)cccc45)n3)ccc2c1 ; c1ccc2cc(-c3nc(-c4ccc5ccccc5c4)nc(-c4ccc5c(-c6cccc7oc8cccnc8c67)cccc5c4)n3)ccc2c1 ; c1ccc2cc(-c3nc(-c4ccc(-c5cccc6oc7cccnc7c56)c5ccccc45)nc(-c4cccc5oc6ccccc6c45)n3)ccc2c1 ; c1ccc2cc(-c3nc(-c4cccc5c(-c6cccc7oc8cccnc8c67)cccc45)nc(-c4cccc5oc6ccccc6c45)n3)ccc2c1]

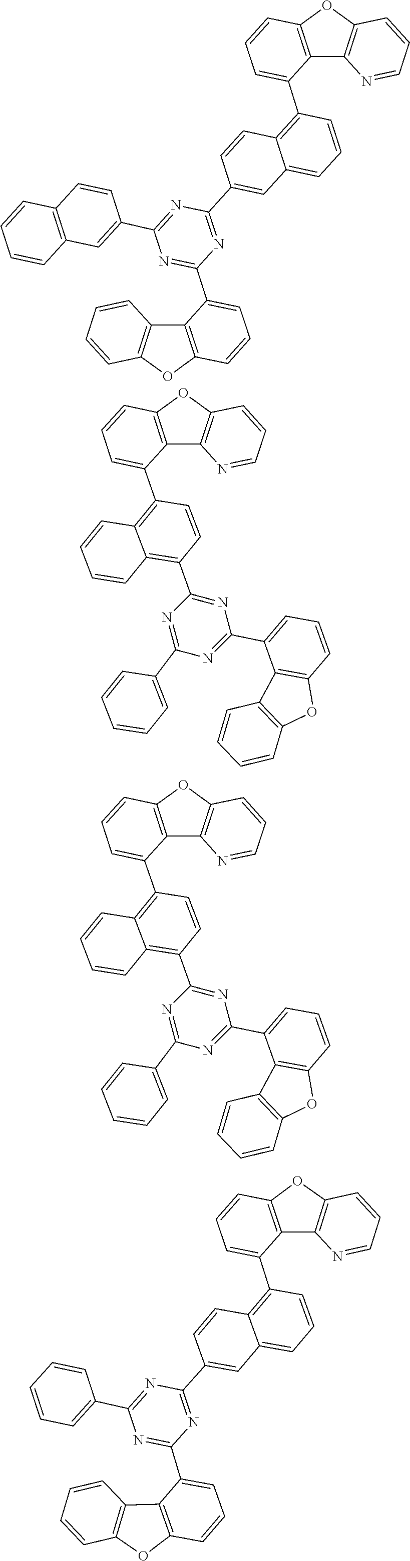

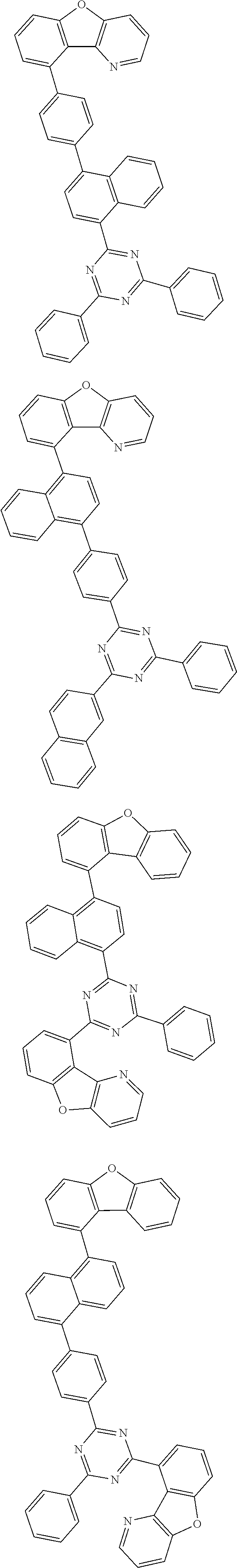

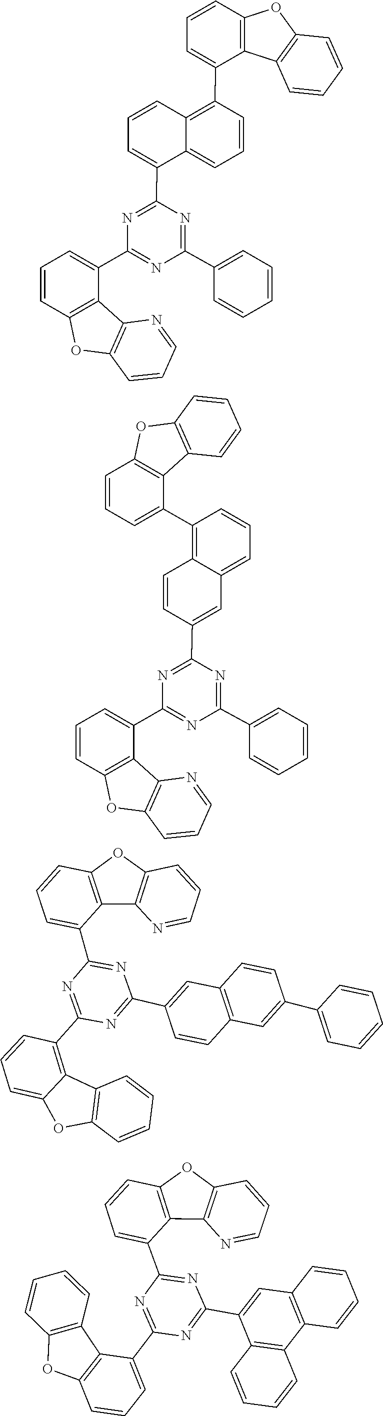

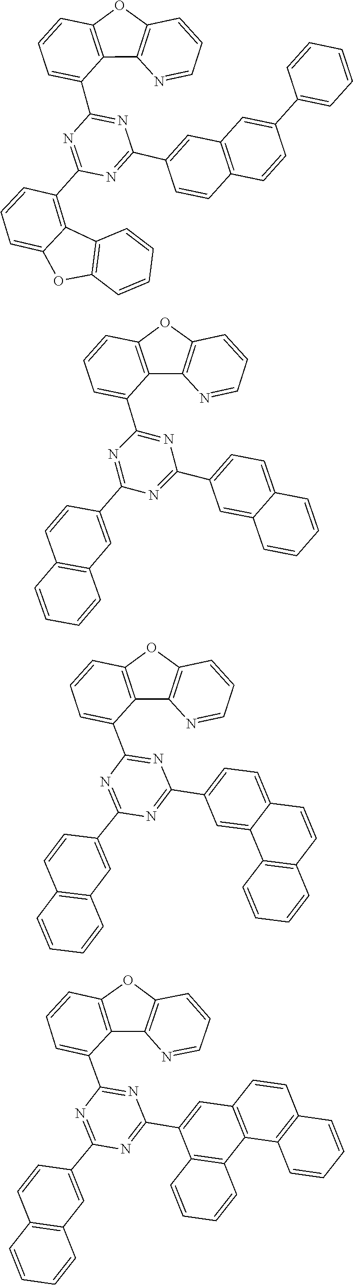

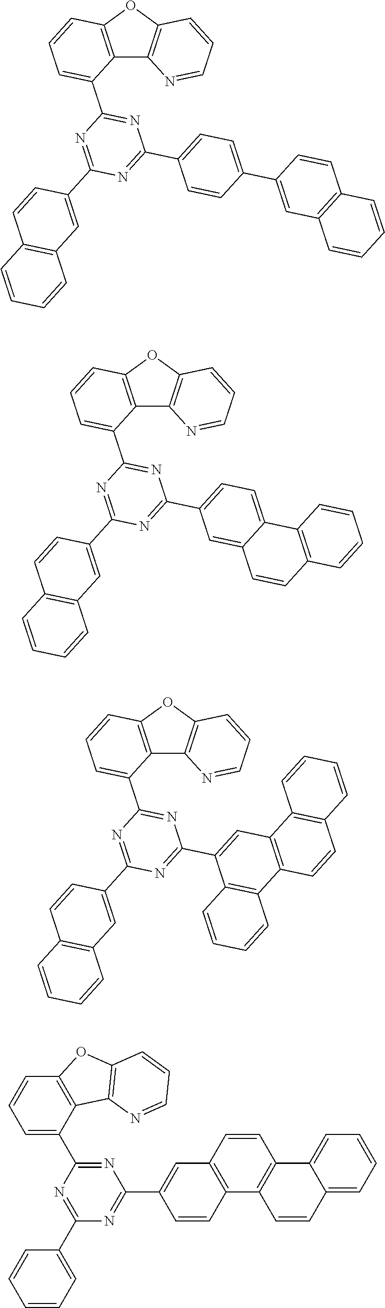 [SMILES: c1ccc2cc(-c3ccc(-c4nc(-c5ccc6ccccc6c5)nc(-c5cccc6oc7cccnc7c56)n4)cc3)ccc2c1 ; c1ccc2cc(-c3nc(-c4ccc5c(ccc6ccccc65)c4)nc(-c4cccc5oc6cccnc6c45)n3)ccc2c1 ; c1ccc2cc(-c3nc(-c4cc5c6ccccc6ccc5c5ccccc45)nc(-c4cccc5oc6cccnc6c45)n3)ccc2c1 ; c1ccc(-c2nc(-c3ccc4c(ccc5c6ccccc6ccc45)c3)nc(-c3cccc4oc5cccnc5c34)n2)cc1]

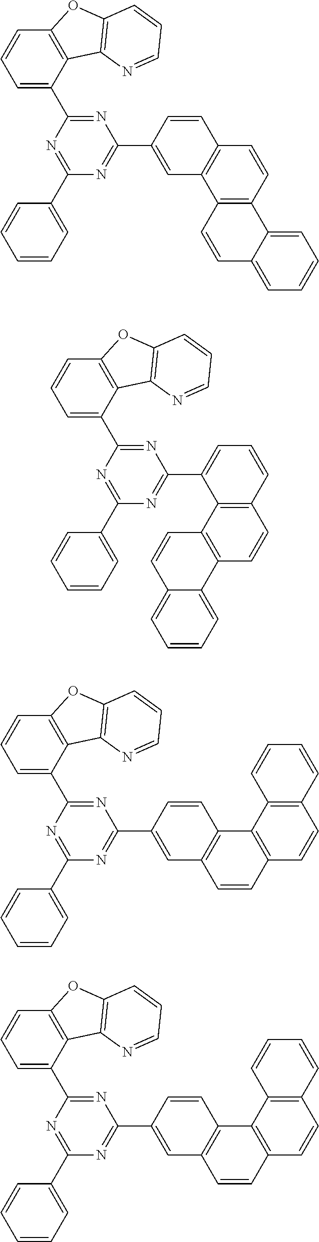

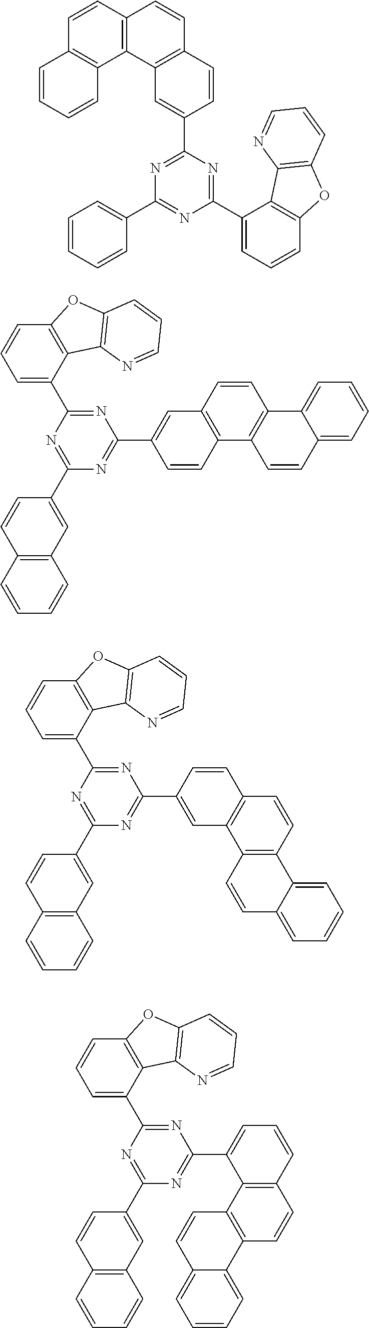

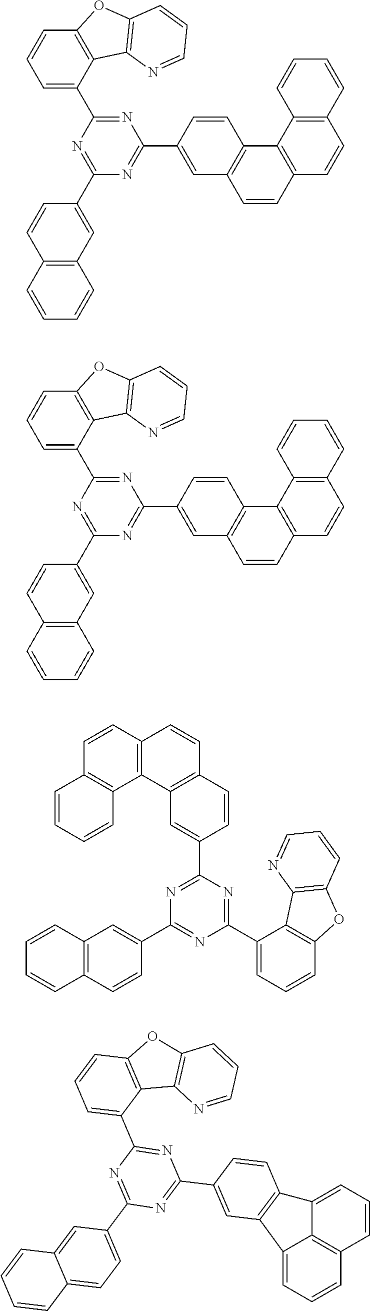 [SMILES: c1ccc2cc(-c3nc(-c4ccc5c(ccc6ccc7ccccc7c65)c4)nc(-c4cccc5oc6cccnc6c45)n3)ccc2c1 ; c1ccc2cc(-c3nc(-c4ccc5c(ccc6ccc7ccccc7c65)c4)nc(-c4cccc5oc6cccnc6c45)n3)ccc2c1 ; c1ccc2cc(-c3nc(-c4ccc5ccc6ccc7ccccc7c6c5c4)nc(-c4cccc5oc6cccnc6c45)n3)ccc2c1 ; c1ccc2cc(-c3nc(-c4ccc5c(c4)-c4cccc6cccc-5c46)nc(-c4cccc5oc6cccnc6c45)n3)ccc2c1]

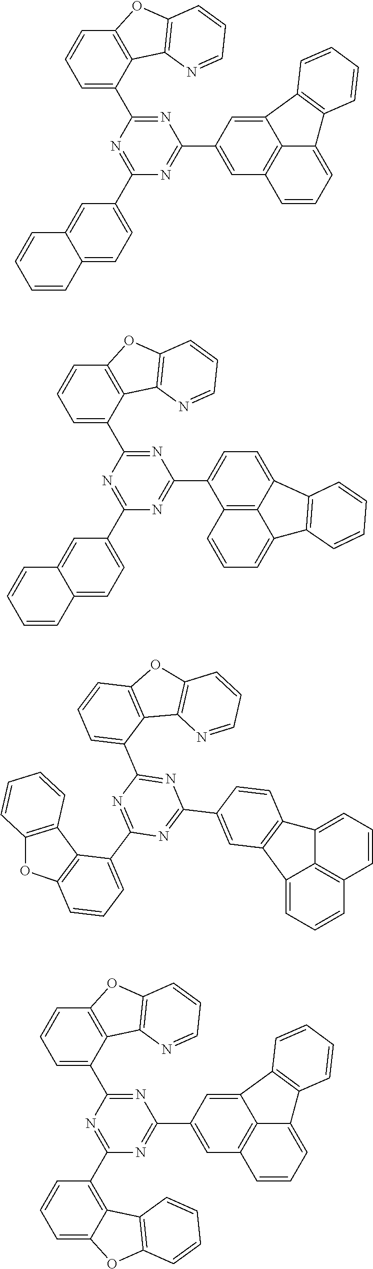 [SMILES: c1ccc2c(c1)-c1cccc3cc(-c4nc(-c5ccc6ccccc6c5)nc(-c5cccc6oc7cccnc7c56)n4)cc-2c13 ; c1ccc2c(c1)-c1cccc3c(-c4nc(-c5ccc6ccccc6c5)nc(-c5cccc6oc7cccnc7c56)n4)ccc-2c13 ; c1cc2c3c(cccc3c1)-c1cc(-c3nc(-c4cccc5oc6ccccc6c45)nc(-c4cccc5oc6cccnc6c45)n3)ccc1-2 ; c1ccc2c(c1)-c1cccc3cc(-c4nc(-c5cccc6oc7ccccc7c56)nc(-c5cccc6oc7cccnc7c56)n4)cc-2c13]

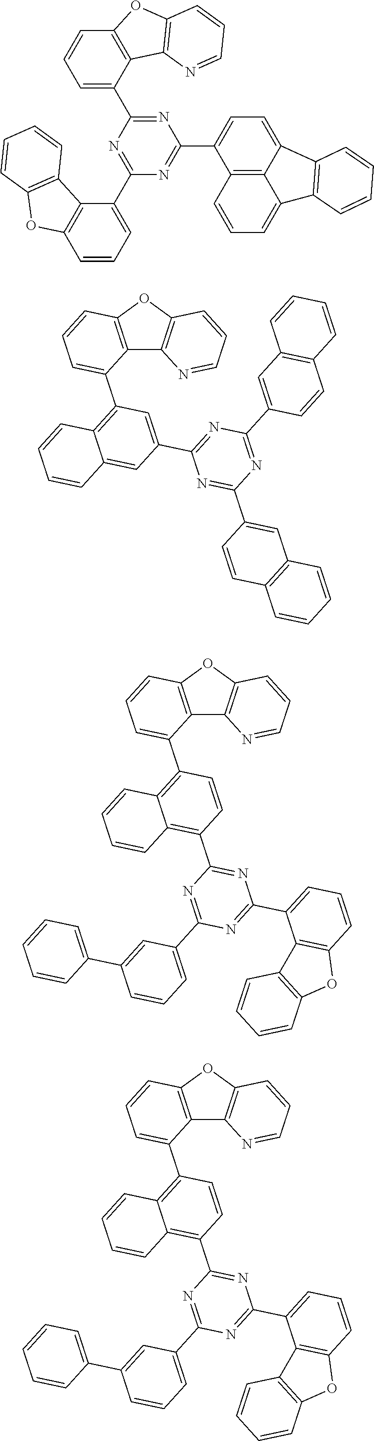

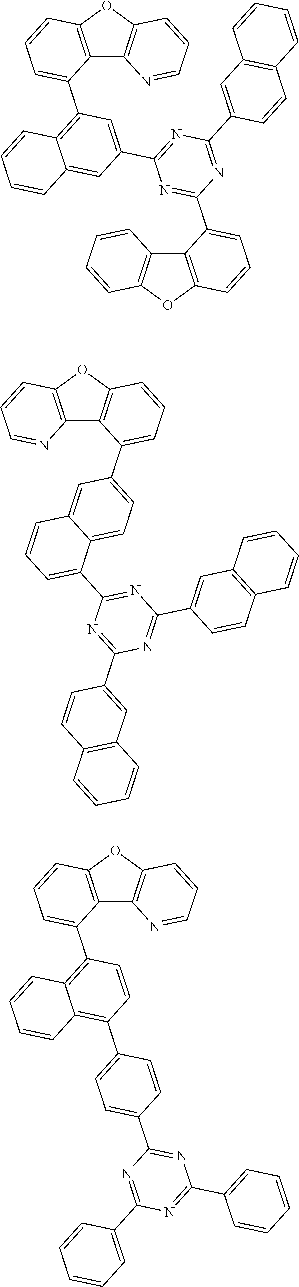 [SMILES: c1ccc2cc(-c3nc(-c4cc(-c5cccc6oc7cccnc7c56)c5ccccc5c4)nc(-c4cccc5oc6ccccc6c45)n3)ccc2c1 ; c1ccc2cc(-c3nc(-c4ccc5ccccc5c4)nc(-c4cccc5cc(-c6cccc7oc8cccnc8c67)ccc45)n3)ccc2c1 ; c1ccc(-c2nc(-c3ccccc3)nc(-c3ccc(-c4ccc(-c5cccc6oc7cccnc7c56)c5ccccc45)cc3)n2)cc1]

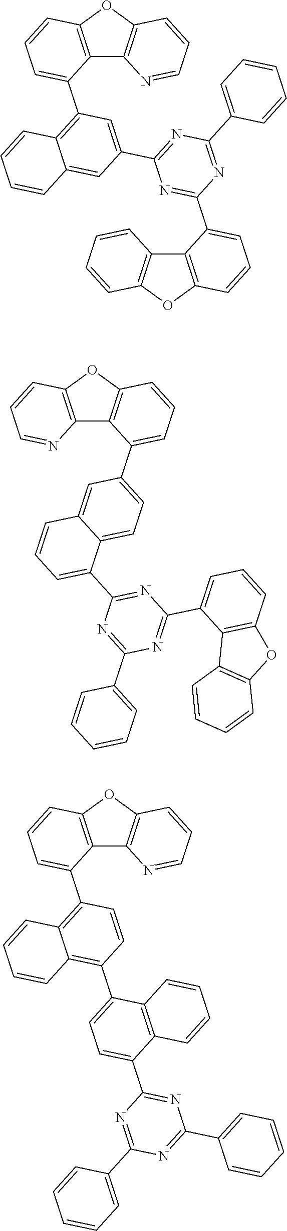 [SMILES: c1ccc(-c2nc(-c3cc(-c4cccc5oc6cccnc6c45)c4ccccc4c3)nc(-c3cccc4oc5ccccc5c34)n2)cc1 ; c1ccc(-c2nc(-c3cccc4cc(-c5cccc6oc7cccnc7c56)ccc34)nc(-c3cccc4oc5ccccc5c34)n2)cc1 ; c1ccc(-c2nc(-c3ccccc3)nc(-c3ccc(-c4ccc(-c5cccc6oc7cccnc7c56)c5ccccc45)c4ccccc34)n2)cc1]

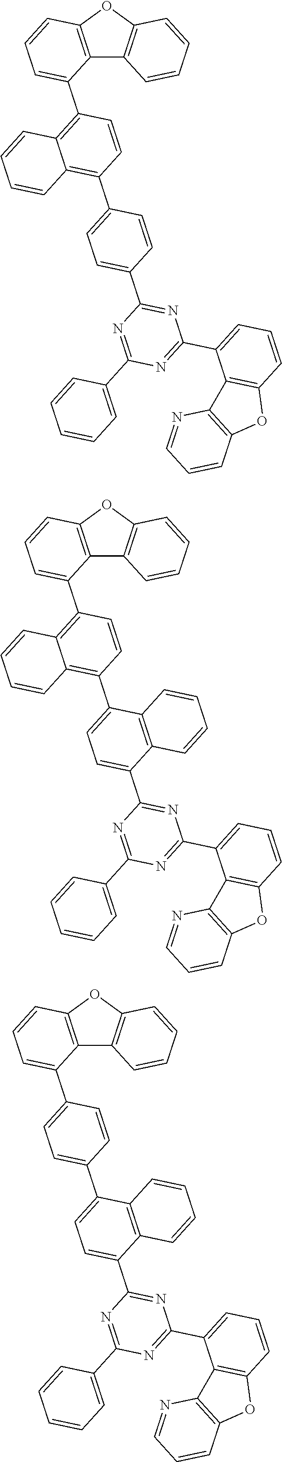 [SMILES: c1ccc(-c2nc(-c3ccc(-c4ccc(-c5cccc6oc7ccccc7c56)c5ccccc45)cc3)nc(-c3cccc4oc5cccnc5c34)n2)cc1 ; c1ccc(-c2nc(-c3ccc(-c4ccc(-c5cccc6oc7ccccc7c56)c5ccccc45)c4ccccc34)nc(-c3cccc4oc5cccnc5c34)n2)cc1 ; c1ccc(-c2nc(-c3ccc(-c4ccc(-c5cccc6oc7ccccc7c56)cc4)c4ccccc34)nc(-c3cccc4oc5cccnc5c34)n2)cc1]

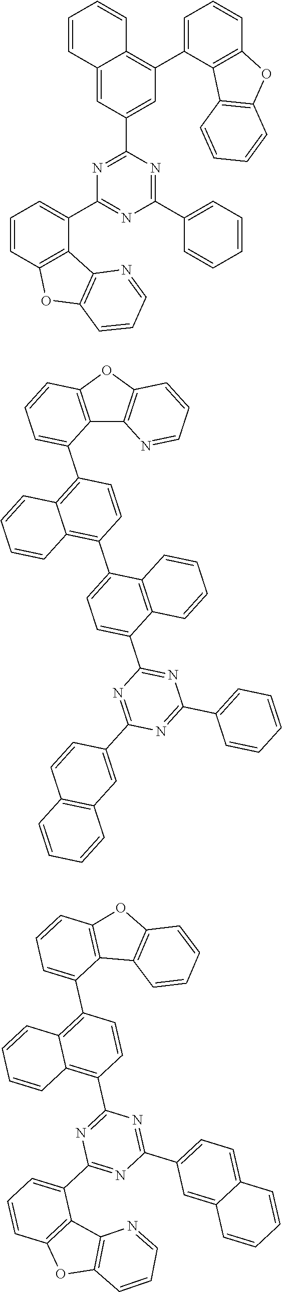

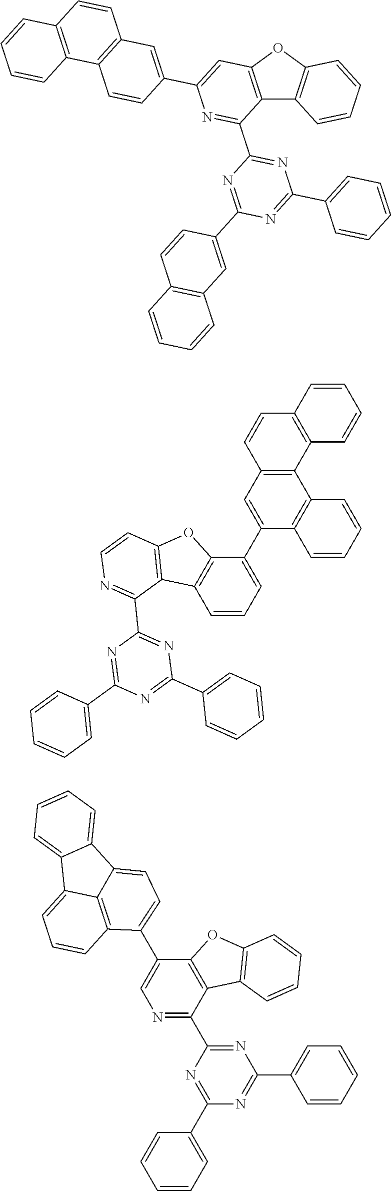

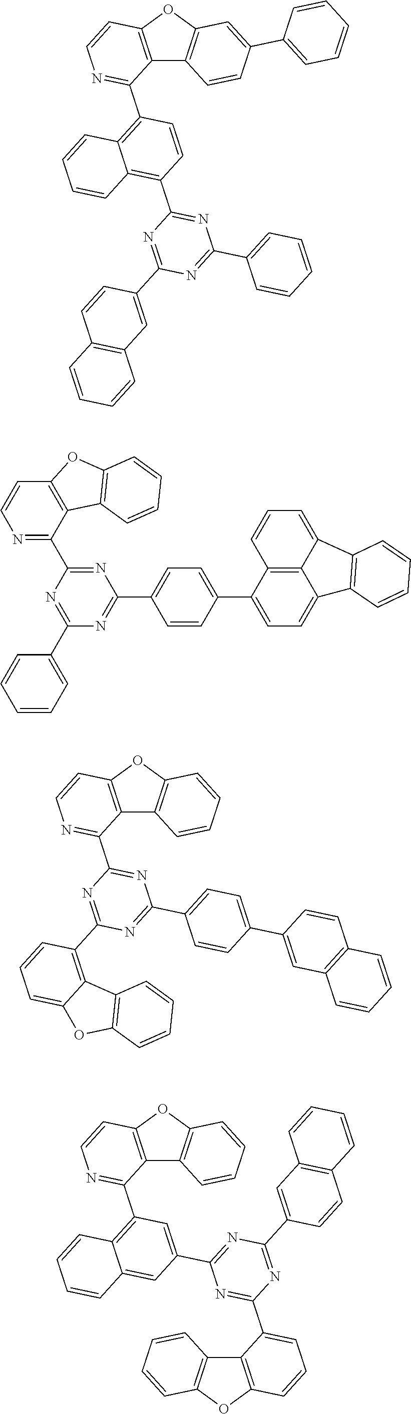 [SMILES: c1ccc(-c2ccc3c(c2)oc2ccnc(-c4ccc(-c5nc(-c6ccccc6)nc(-c6ccc7ccccc7c6)n5)c5ccccc45)c23)cc1 ; c1ccc(-c2nc(-c3ccc(-c4ccc5c6c(cccc46)-c4ccccc4-5)cc3)nc(-c3nccc4oc5ccccc5c34)n2)cc1 ; c1ccc2cc(-c3ccc(-c4nc(-c5cccc6oc7ccccc7c56)nc(-c5nccc6oc7ccccc7c56)n4)cc3)ccc2c1 ; c1ccc2cc(-c3nc(-c4cc(-c5nccc6oc7ccccc7c56)c5ccccc5c4)nc(-c4cccc5oc6ccccc6c45)n3)ccc2c1]

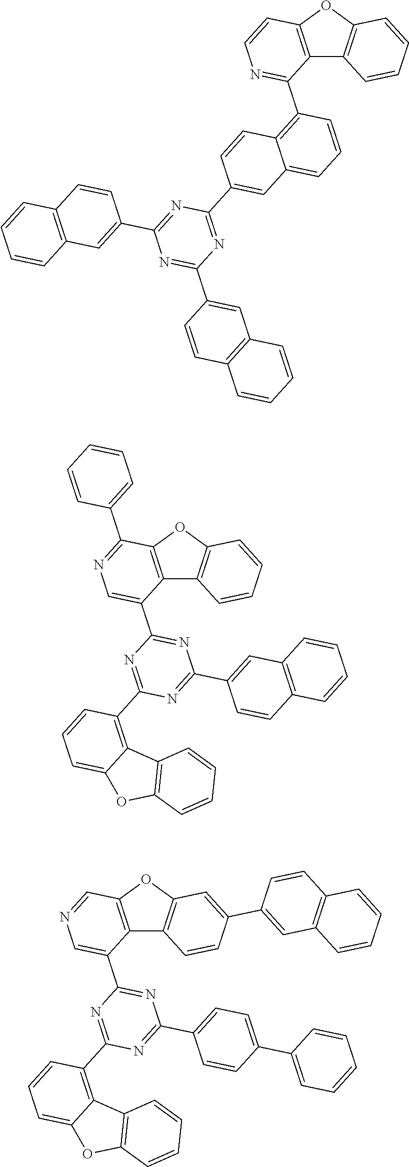 [SMILES: c1ccc2cc(-c3nc(-c4ccc5ccccc5c4)nc(-c4ccc5c(-c6nccc7oc8ccccc8c67)cccc5c4)n3)ccc2c1 ; c1ccc(-c2ncc(-c3nc(-c4ccc5ccccc5c4)nc(-c4cccc5oc6ccccc6c45)n3)c3c2oc2ccccc23)cc1 ; c1ccc(-c2ccc(-c3nc(-c4cccc5oc6ccccc6c45)nc(-c4cncc5oc6cc(-c7ccc8ccccc8c7)ccc6c45)n3)cc2)cc1]

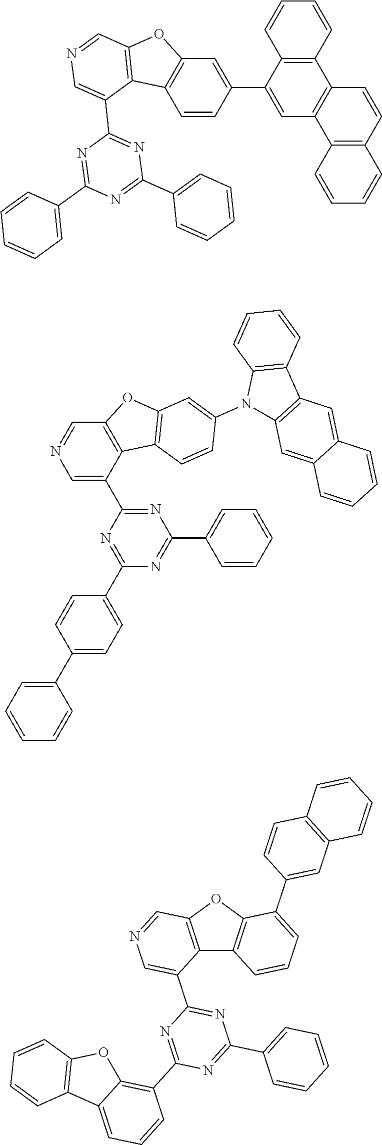 [SMILES: c1ccc(-c2nc(-c3ccccc3)nc(-c3cncc4oc5cc(-c6cc7c8ccccc8ccc7c7ccccc67)ccc5c34)n2)cc1 ; c1ccc(-c2ccc(-c3nc(-c4ccccc4)nc(-c4cncc5oc6cc(-n7c8ccccc8c8cc9ccccc9cc87)ccc6c45)n3)cc2)cc1 ; c1ccc(-c2nc(-c3cccc4c3oc3ccccc34)nc(-c3cncc4oc5c(-c6ccc7ccccc7c6)cccc5c34)n2)cc1]

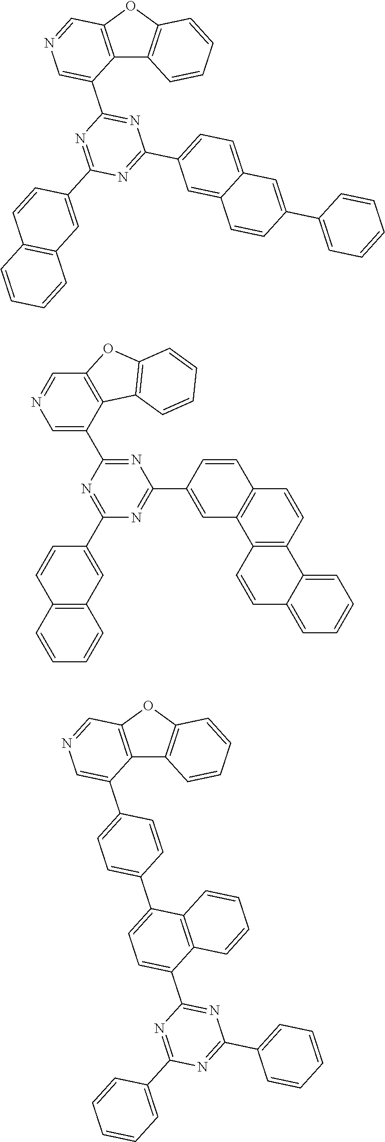 [SMILES: c1ccc(-c2ccc3cc(-c4nc(-c5ccc6ccccc6c5)nc(-c5cncc6oc7ccccc7c56)n4)ccc3c2)cc1 ; c1ccc2cc(-c3nc(-c4ccc5ccc6c7ccccc7ccc6c5c4)nc(-c4cncc5oc6ccccc6c45)n3)ccc2c1 ; c1ccc(-c2nc(-c3ccccc3)nc(-c3ccc(-c4ccc(-c5cncc6oc7ccccc7c56)cc4)c4ccccc34)n2)cc1]

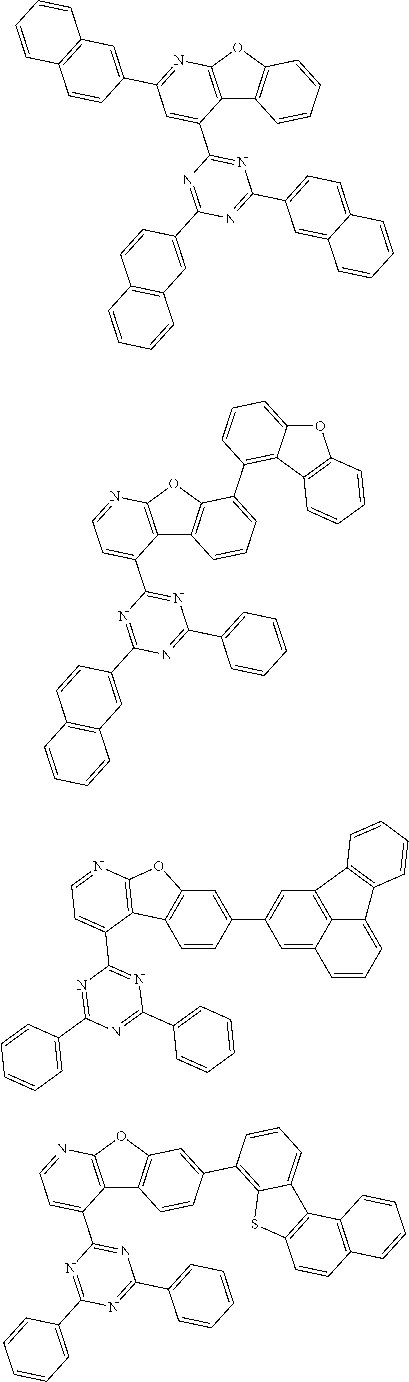 [SMILES: c1ccc2cc(-c3cc(-c4nc(-c5ccc6ccccc6c5)nc(-c5ccc6ccccc6c5)n4)c4c(n3)oc3ccccc34)ccc2c1 ; c1ccc(-c2nc(-c3ccc4ccccc4c3)nc(-c3ccnc4oc5c(-c6cccc7oc8ccccc8c67)cccc5c34)n2)cc1 ; c1ccc(-c2nc(-c3ccccc3)nc(-c3ccnc4oc5cc(-c6cc7c8c(cccc8c6)-c6ccccc6-7)ccc5c34)n2)cc1 ; c1ccc(-c2nc(-c3ccccc3)nc(-c3ccnc4oc5cc(-c6cccc7c6sc6ccc8ccccc8c67)ccc5c34)n2)cc1]

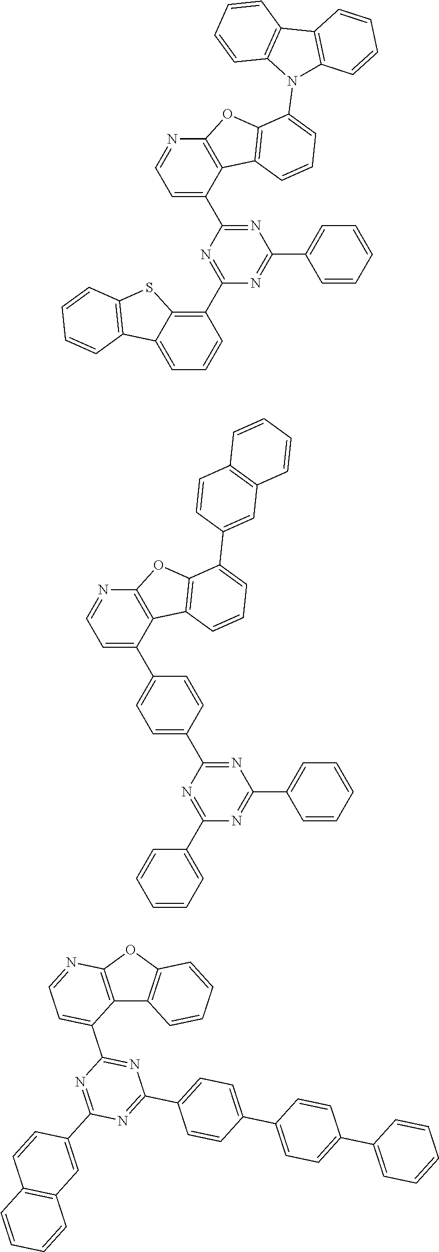 [SMILES: c1ccc(-c2nc(-c3cccc4c3sc3ccccc34)nc(-c3ccnc4oc5c(-n6c7ccccc7c7ccccc76)cccc5c34)n2)cc1 ; c1ccc(-c2nc(-c3ccccc3)nc(-c3ccc(-c4ccnc5oc6c(-c7ccc8ccccc8c7)cccc6c45)cc3)n2)cc1 ; c1ccc(-c2ccc(-c3ccc(-c4nc(-c5ccc6ccccc6c5)nc(-c5ccnc6oc7ccccc7c56)n4)cc3)cc2)cc1]

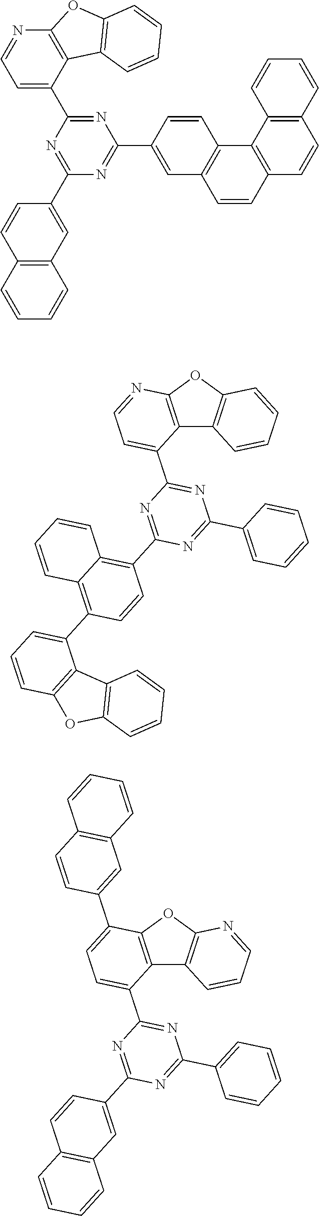

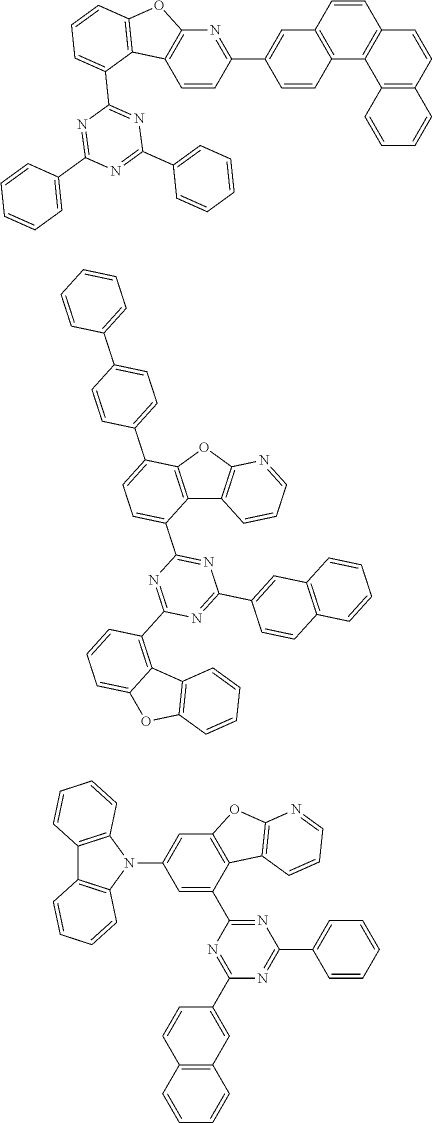 [SMILES: c1ccc(-c2nc(-c3ccccc3)nc(-c3cccc4oc5nc(-c6ccc7c(ccc8ccc9ccccc9c87)c6)ccc5c34)n2)cc1 ; c1ccc(-c2ccc(-c3ccc(-c4nc(-c5ccc6ccccc6c5)nc(-c5cccc6oc7ccccc7c56)n4)c4c3oc3ncccc34)cc2)cc1 ; c1ccc(-c2nc(-c3ccc4ccccc4c3)nc(-c3cc(-n4c5ccccc5c5ccccc54)cc4oc5ncccc5c34)n2)cc1]

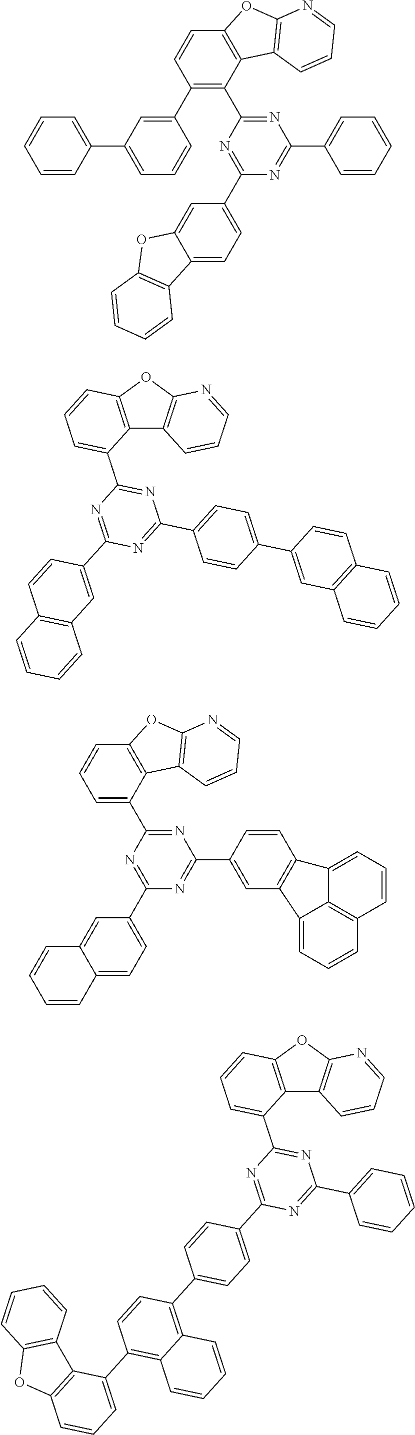

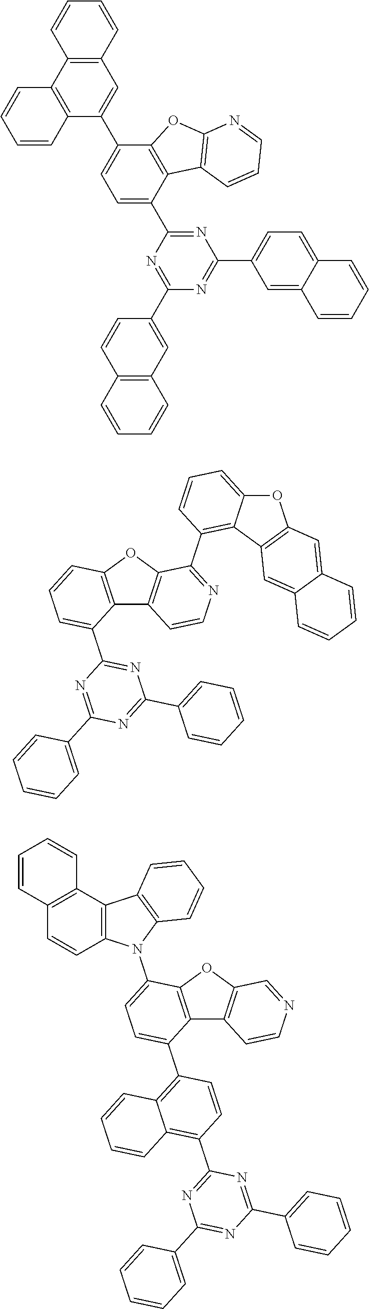

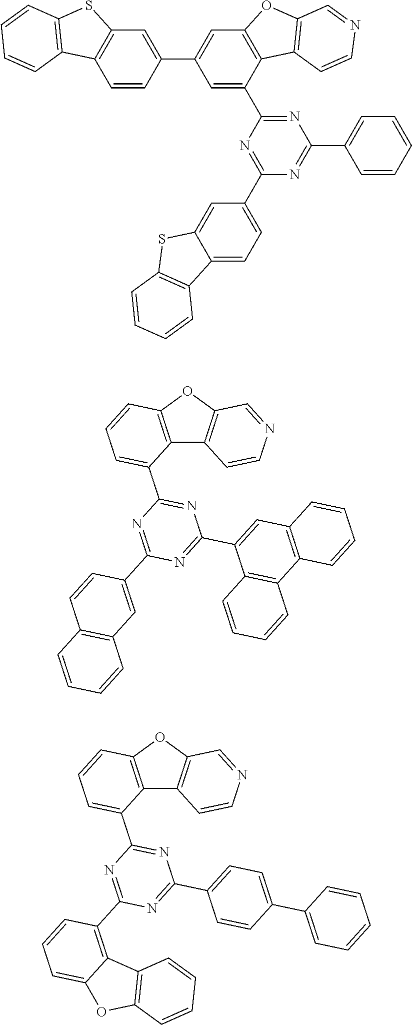 [SMILES: c1ccc(-c2nc(-c3ccc4c(c3)sc3ccccc34)nc(-c3cc(-c4ccc5c(c4)sc4ccccc45)cc4oc5cnccc5c34)n2)cc1 ; c1ccc2cc(-c3nc(-c4cc5ccccc5c5ccccc45)nc(-c4cccc5oc6cnccc6c45)n3)ccc2c1 ; c1ccc(-c2ccc(-c3nc(-c4cccc5oc6ccccc6c45)nc(-c4cccc5oc6cnccc6c45)n3)cc2)cc1]

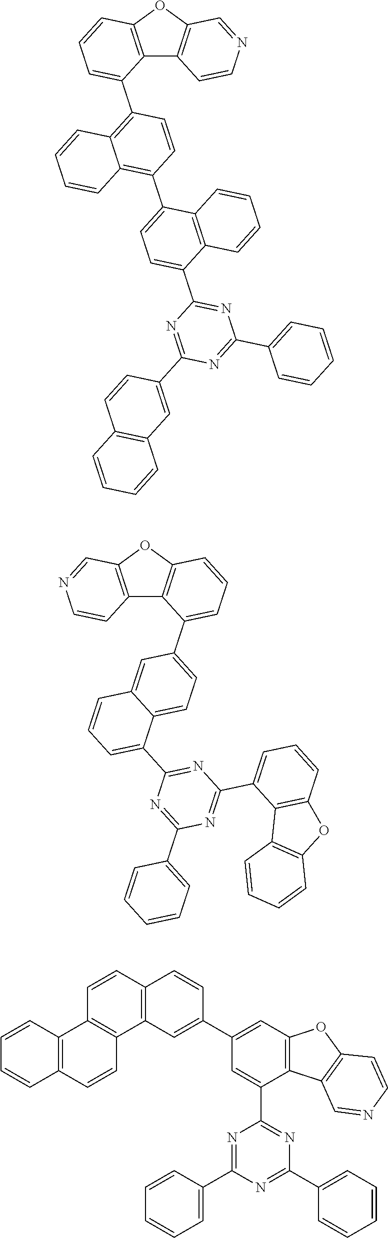 [SMILES: c1ccc(-c2nc(-c3ccc4ccccc4c3)nc(-c3ccc(-c4ccc(-c5cccc6oc7cnccc7c56)c5ccccc45)c4ccccc34)n2)cc1 ; c1ccc(-c2nc(-c3cccc4cc(-c5cccc6oc7cnccc7c56)ccc34)nc(-c3cccc4oc5ccccc5c34)n2)cc1 ; c1ccc(-c2nc(-c3ccccc3)nc(-c3cc(-c4ccc5ccc6c7ccccc7ccc6c5c4)cc4oc5ccncc5c34)n2)cc1]

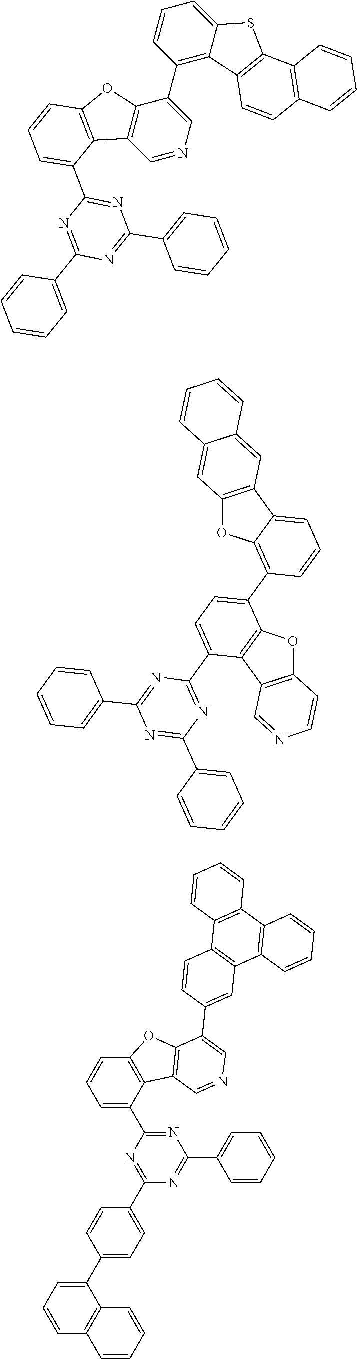

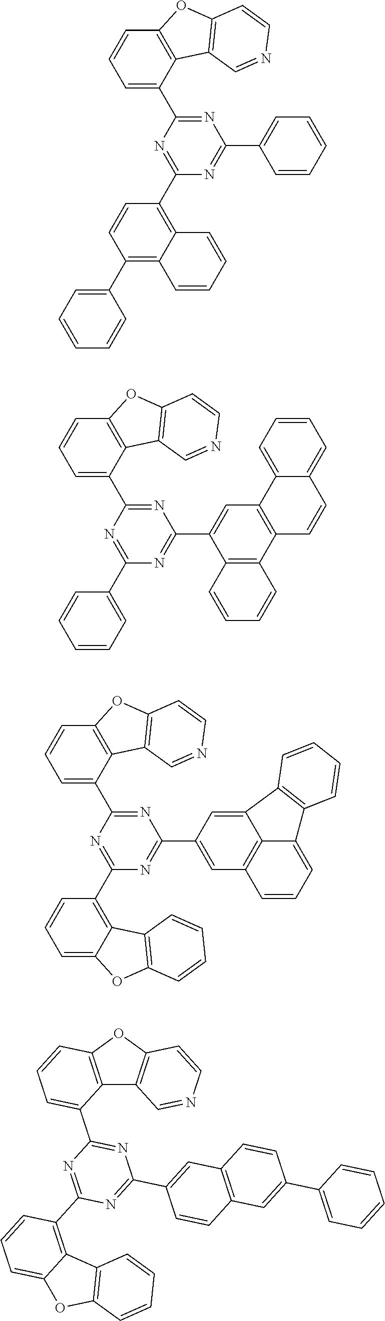 [SMILES: c1ccc(-c2nc(-c3ccc(-c4ccccc4)c4ccccc34)nc(-c3cccc4oc5ccncc5c34)n2)cc1 ; c1ccc(-c2nc(-c3cc4c5ccccc5ccc4c4ccccc34)nc(-c3cccc4oc5ccncc5c34)n2)cc1 ; c1ccc2c(c1)-c1cccc3cc(-c4nc(-c5cccc6oc7ccccc7c56)nc(-c5cccc6oc7ccncc7c56)n4)cc-2c13 ; c1ccc(-c2ccc3cc(-c4nc(-c5cccc6oc7ccccc7c56)nc(-c5cccc6oc7ccncc7c56)n4)ccc3c2)cc1]

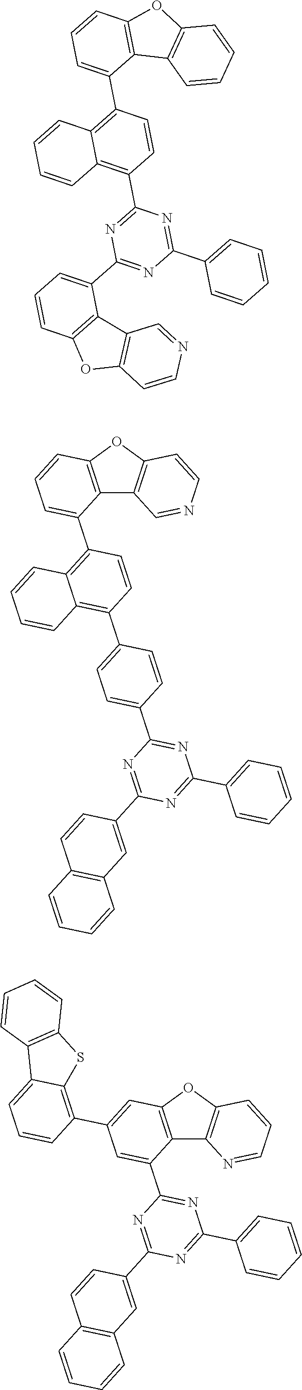 [SMILES: c1ccc(-c2nc(-c3ccc(-c4cccc5oc6ccccc6c45)c4ccccc34)nc(-c3cccc4oc5ccncc5c34)n2)cc1 ; c1ccc(-c2nc(-c3ccc(-c4ccc(-c5cccc6oc7ccncc7c56)c5ccccc45)cc3)nc(-c3ccc4ccccc4c3)n2)cc1 ; c1ccc(-c2nc(-c3ccc4ccccc4c3)nc(-c3cc(-c4cccc5c4sc4ccccc45)cc4oc5cccnc5c34)n2)cc1]

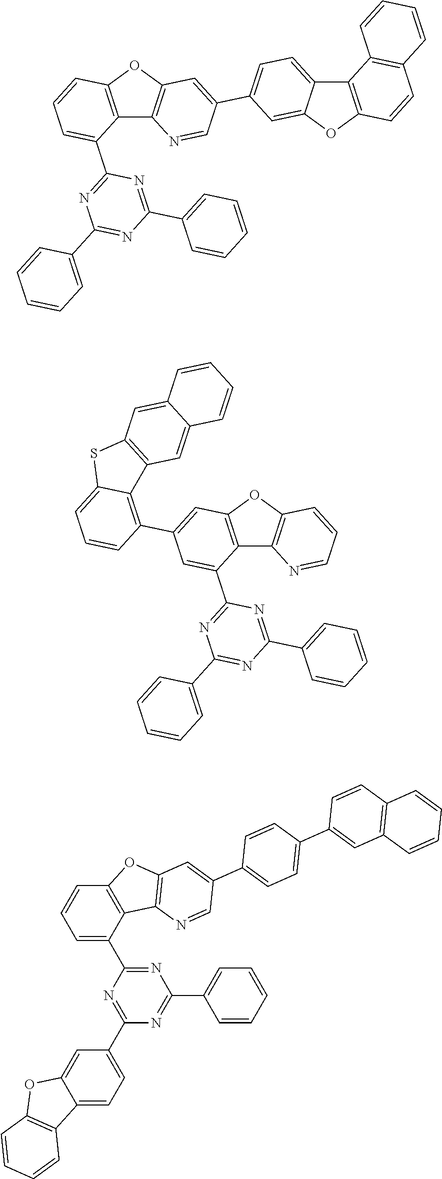 [SMILES: c1ccc(-c2nc(-c3ccccc3)nc(-c3cccc4oc5cc(-c6ccc7c(c6)oc6ccc8ccccc8c67)cnc5c34)n2)cc1 ; c1ccc(-c2nc(-c3ccccc3)nc(-c3cc(-c4cccc5sc6cc7ccccc7cc6c45)cc4oc5cccnc5c34)n2)cc1 ; c1ccc(-c2nc(-c3ccc4c(c3)oc3ccccc34)nc(-c3cccc4oc5cc(-c6ccc(-c7ccc8ccccc8c7)cc6)cnc5c34)n2)cc1]

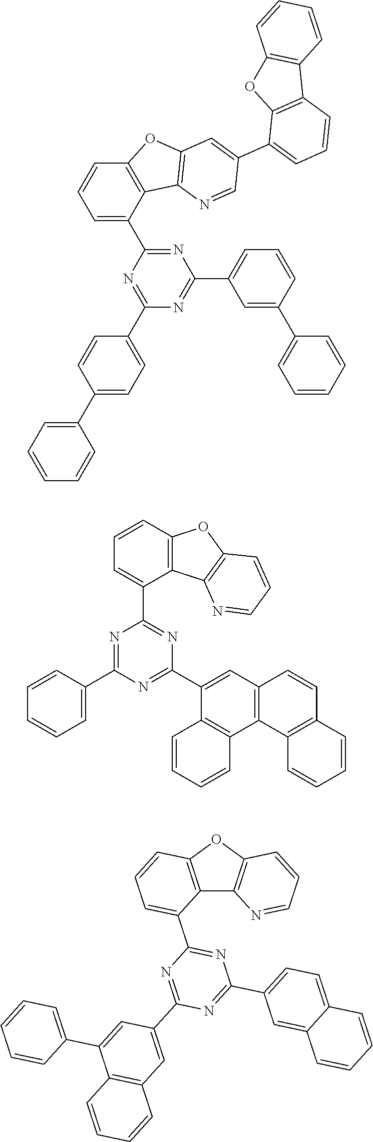 [SMILES: c1ccc(-c2ccc(-c3nc(-c4cccc(-c5ccccc5)c4)nc(-c4cccc5oc6cc(-c7cccc8c7oc7ccccc78)cnc6c45)n3)cc2)cc1 ; c1ccc(-c2nc(-c3cc4ccc5ccccc5c4c4ccccc34)nc(-c3cccc4oc5cccnc5c34)n2)cc1 ; c1ccc(-c2cc(-c3nc(-c4ccc5ccccc5c4)nc(-c4cccc5oc6cccnc6c45)n3)cc3ccccc23)cc1]

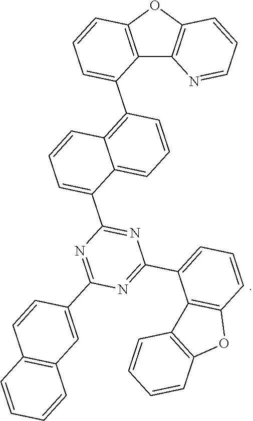 [SMILES: c1ccc2cc(-c3nc(-c4cccc5c(-c6cccc7oc8cccnc8c67)cccc45)nc(-c4cccc5oc6ccccc6c45)n3)ccc2c1]

7. The organic light emitting device according to claim 1, wherein:
L₄ and L₅ are each independently a single bond, phenylene, biphenyldiyl, or phenylene substituted with 4 deuteriums.

8. The organic light emitting device according to claim 1, wherein:
A and B are each independently hydrogen, phenyl that is unsubstituted or substituted with 1 to 5 deuteriums or 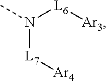
provided that at least one of A and B is

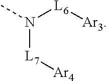

9. The organic light emitting device according to claim 1, wherein:
A is 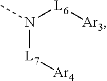
and
B is hydrogen or phenyl that is unsubstituted or substituted with 1 to 5 deuteriums.

10. The organic light emitting device according to claim 1, wherein:
L₆ and L₇ are each independently a single bond, phenylene, biphenyldiyl, or phenylene substituted with 4 deuteriums.

11. The organic light emitting device according to claim 1, wherein:
Ar₃ and Ar₄ are each independently phenyl, biphenylyl, terphenylyl, naphthyl, phenyl naphthyl, phenanthrenyl, triphenylenyl, phenyl phenanthrenyl, dimethylfluorenyl, diphenylfluorenyl, dibenzofuranyl, dibenzothiophenyl, methyl dibenzofluorenyl, carbazolyl, phenyl carbazolyl, phenyl substituted with 5 deuteriums, biphenylyl substituted with 4 deuteriums, biphenylyl substituted with 9 deuteriums, or terphenylyl substituted with 4 deuteriums.

12. The organic light emitting device according to claim 1, wherein:
the compound of Chemical Formula 2 is any one compound selected from the group consisting of: 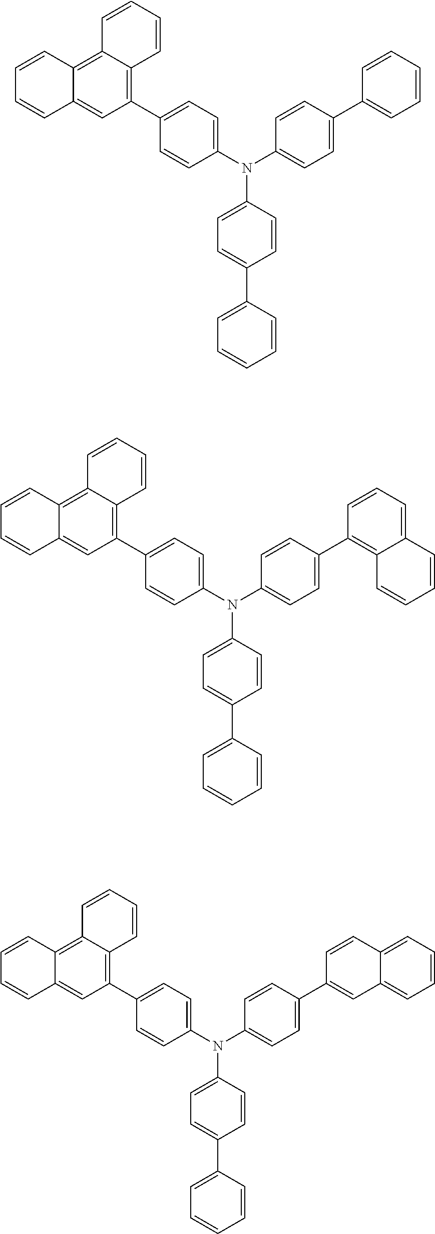 [SMILES: c1ccc(-c2ccc(N(c3ccc(-c4ccccc4)cc3)c3ccc(-c4cc5ccccc5c5ccccc45)cc3)cc2)cc1 ; c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccc5ccccc5c4)cc3)c3ccc(-c4cc5ccccc5c5ccccc45)cc3)cc2)cc1] 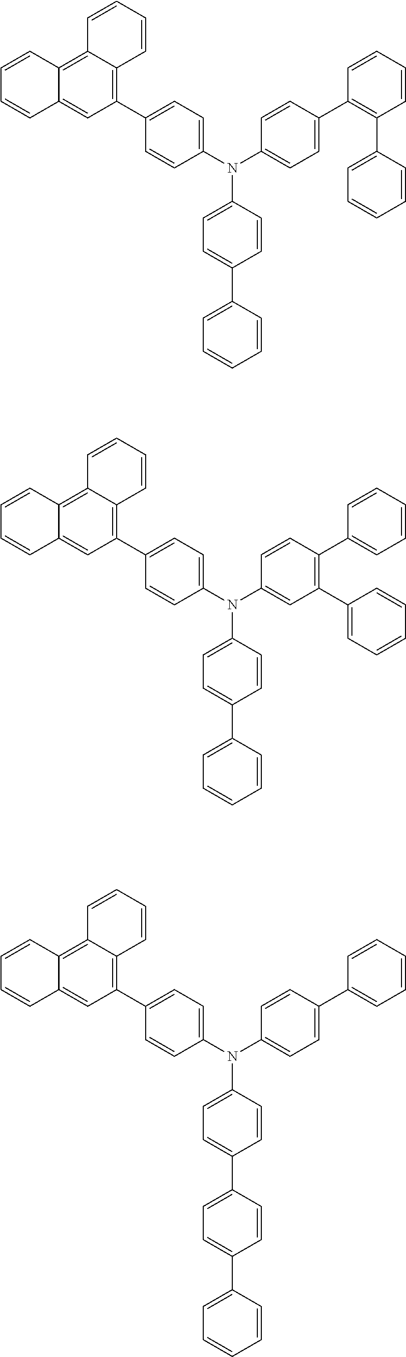 [SMILES: c1ccc(-c2ccc(N(c3ccc(-c4ccccc4-c4ccccc4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c(-c4ccccc4)c3)cc2)cc1 ; c1ccc(-c2ccc(-c3ccc(N(c4ccc(-c5ccccc5)cc4)c4ccc(-c5cc6ccccc6c6ccccc56)cc4)cc3)cc2)cc1] 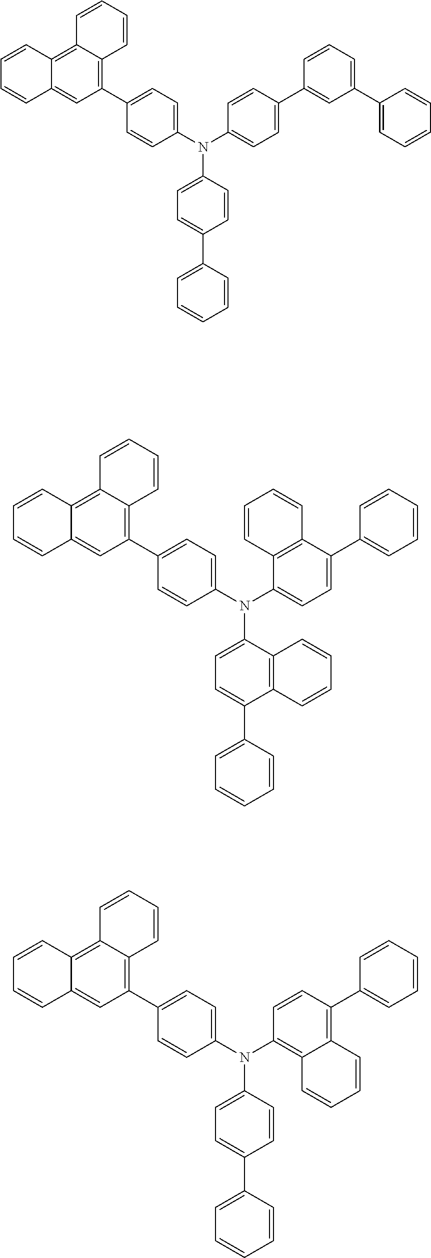 [SMILES: c1ccc(-c2ccc(N(c3ccc(-c4cccc(-c5ccccc5)c4)cc3)c3ccc(-c4cc5ccccc5c5ccccc45)cc3)cc2)cc1 ; c1ccc(-c2ccc(N(c3ccc(-c4cc5ccccc5c5ccccc45)cc3)c3ccc(-c4ccccc4)c4ccccc34)c3ccccc23)cc1 ; c1ccc(-c2ccc(N(c3ccc(-c4cc5ccccc5c5ccccc45)cc3)c3ccc(-c4ccccc4)c4ccccc34)cc2)cc1] 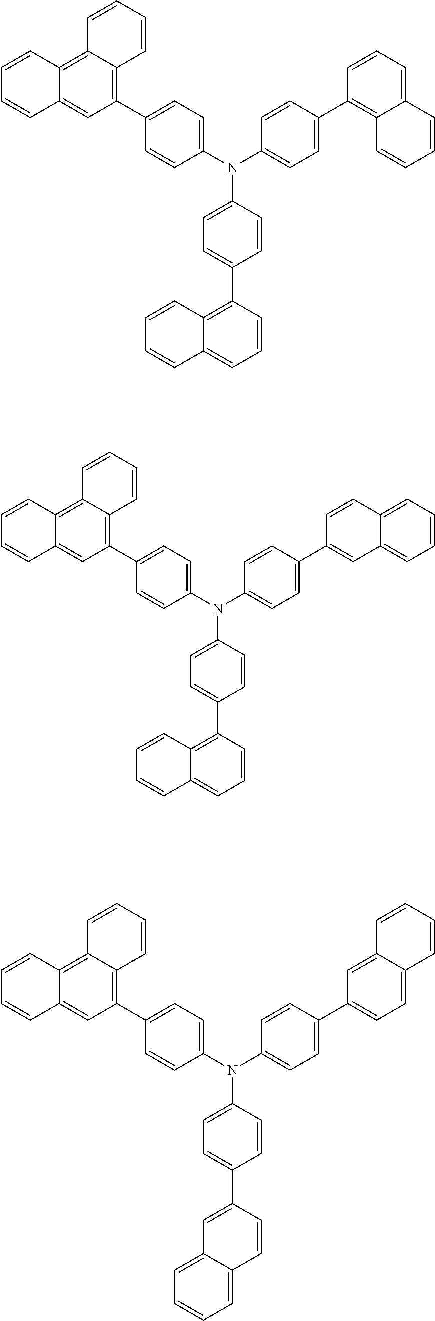 [SMILES: c1ccc2c(-c3ccc(N(c4ccc(-c5cccc6ccccc56)cc4)c4ccc(-c5cc6ccccc6c6ccccc56)cc4)cc3)cccc2c1 ; c1ccc2cc(-c3ccc(N(c4ccc(-c5cccc6ccccc56)cc4)c4ccc(-c5cc6ccccc6c6ccccc56)cc4)cc3)ccc2c1 ; c1ccc2cc(-c3ccc(N(c4ccc(-c5ccc6ccccc6c5)cc4)c4ccc(-c5cc6ccccc6c6ccccc56)cc4)cc3)ccc2c1] 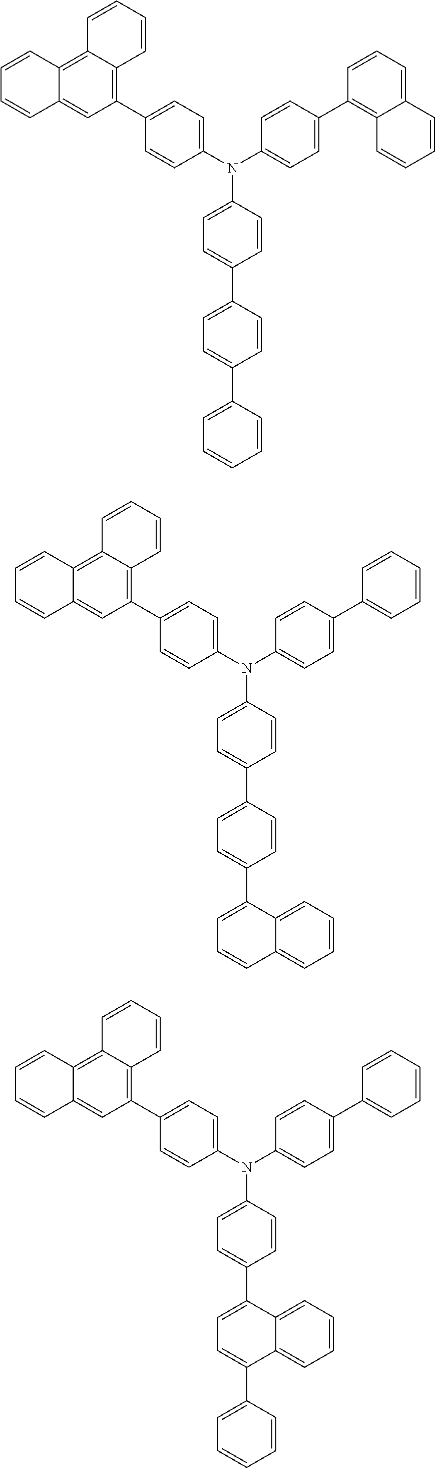 [SMILES: c1ccc(-c2ccc(-c3ccc(N(c4ccc(-c5cccc6ccccc56)cc4)c4ccc(-c5cc6ccccc6c6ccccc56)cc4)cc3)cc2)cc1 ; c1ccc(-c2ccc(N(c3ccc(-c4ccc(-c5cccc6ccccc56)cc4)cc3)c3ccc(-c4cc5ccccc5c5ccccc45)cc3)cc2)cc1 ; c1ccc(-c2ccc(N(c3ccc(-c4ccc(-c5ccccc5)c5ccccc45)cc3)c3ccc(-c4cc5ccccc5c5ccccc45)cc3)cc2)cc1] 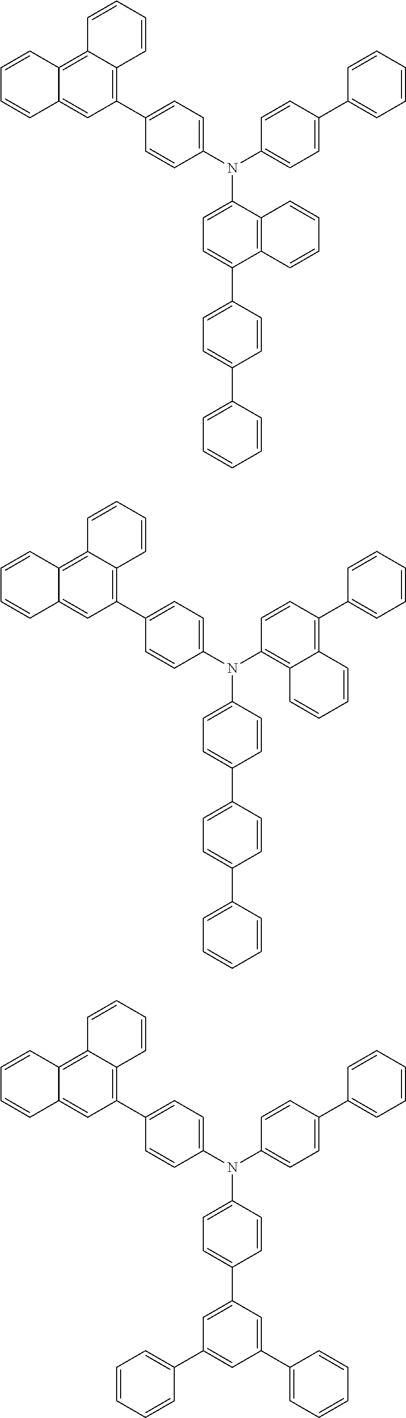 [SMILES: c1ccc(-c2ccc(-c3ccc(N(c4ccc(-c5ccccc5)cc4)c4ccc(-c5cc6ccccc6c6ccccc56)cc4)c4ccccc34)cc2)cc1 ; c1ccc(-c2ccc(-c3ccc(N(c4ccc(-c5cc6ccccc6c6ccccc56)cc4)c4ccc(-c5ccccc5)c5ccccc45)cc3)cc2)cc1 ; c1ccc(-c2ccc(N(c3ccc(-c4cc(-c5ccccc5)cc(-c5ccccc5)c4)cc3)c3ccc(-c4cc5ccccc5c5ccccc45)cc3)cc2)cc1] 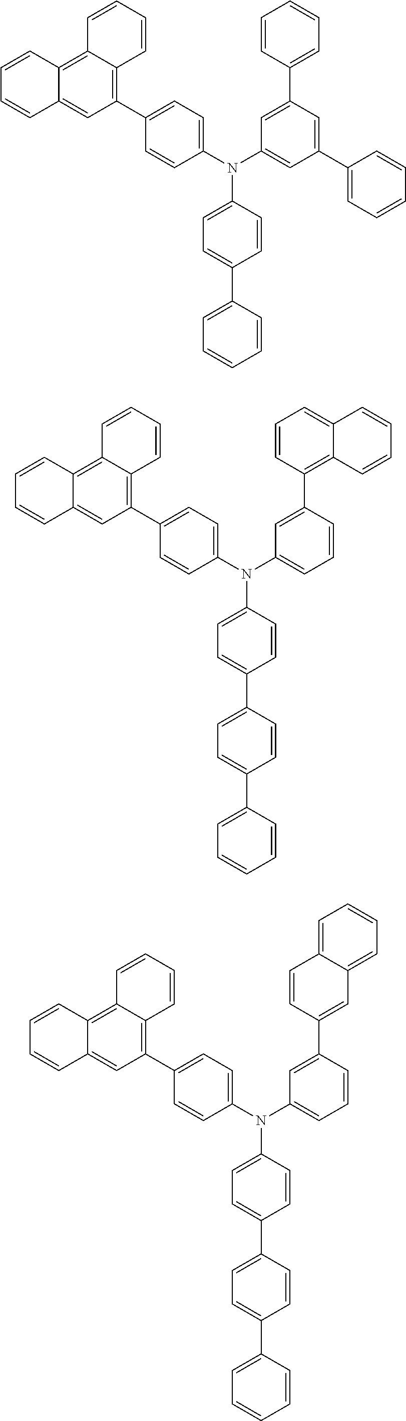 [SMILES: c1ccc(-c2ccc(N(c3ccc(-c4cc5ccccc5c5ccccc45)cc3)c3cc(-c4ccccc4)cc(-c4ccccc4)c3)cc2)cc1 ; c1ccc(-c2ccc(-c3ccc(N(c4ccc(-c5cc6ccccc6c6ccccc56)cc4)c4cccc(-c5cccc6ccccc56)c4)cc3)cc2)cc1 ; c1ccc(-c2ccc(-c3ccc(N(c4ccc(-c5cc6ccccc6c6ccccc56)cc4)c4cccc(-c5ccc6ccccc6c5)c4)cc3)cc2)cc1] 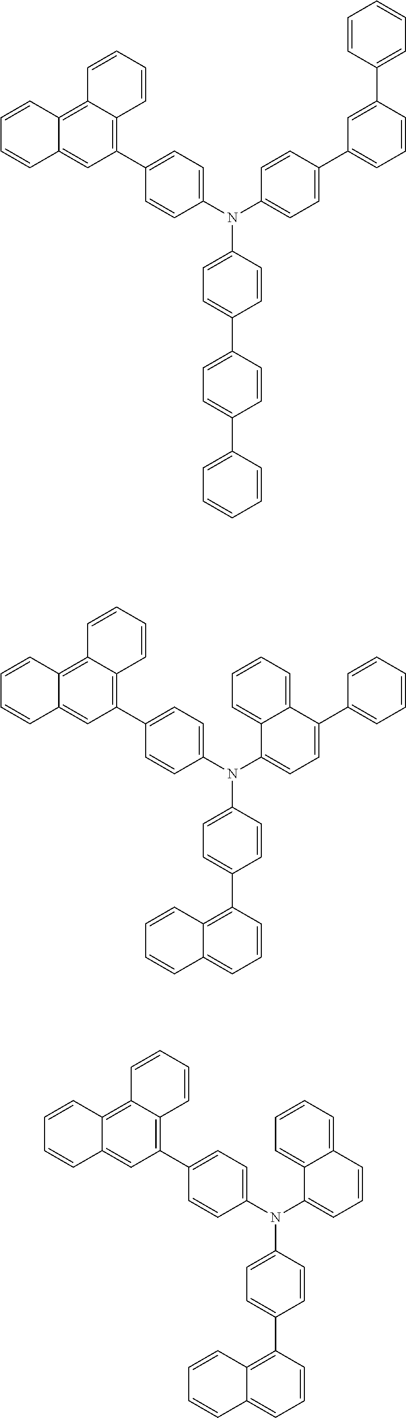 [SMILES: c1ccc(-c2ccc(-c3ccc(N(c4ccc(-c5cccc(-c6ccccc6)c5)cc4)c4ccc(-c5cc6ccccc6c6ccccc56)cc4)cc3)cc2)cc1 ; c1ccc(-c2ccc(N(c3ccc(-c4cccc5ccccc45)cc3)c3ccc(-c4cc5ccccc5c5ccccc45)cc3)c3ccccc23)cc1 ; c1ccc2c(-c3ccc(N(c4ccc(-c5cc6ccccc6c6ccccc56)cc4)c4cccc5ccccc45)cc3)cccc2c1] 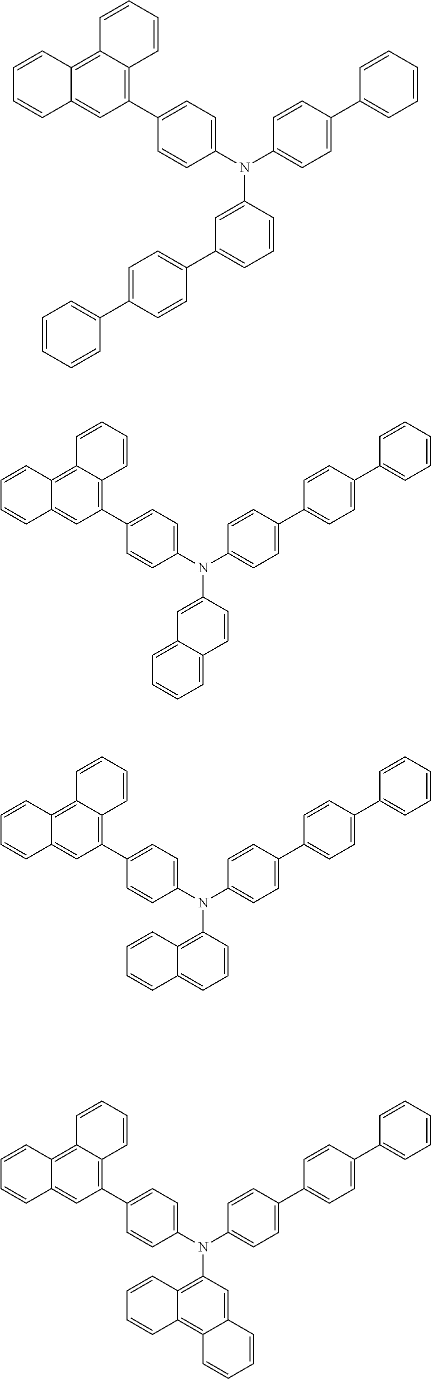 [SMILES: c1ccc(-c2ccc(-c3cccc(N(c4ccc(-c5ccccc5)cc4)c4ccc(-c5cc6ccccc6c6ccccc56)cc4)c3)cc2)cc1 ; c1ccc(-c2ccc(-c3ccc(N(c4ccc(-c5cc6ccccc6c6ccccc56)cc4)c4ccc5ccccc5c4)cc3)cc2)cc1 ; c1ccc(-c2ccc(-c3ccc(N(c4ccc(-c5cc6ccccc6c6ccccc56)cc4)c4cccc5ccccc45)cc3)cc2)cc1 ; c1ccc(-c2ccc(-c3ccc(N(c4ccc(-c5cc6ccccc6c6ccccc56)cc4)c4cc5ccccc5c5ccccc45)cc3)cc2)cc1] 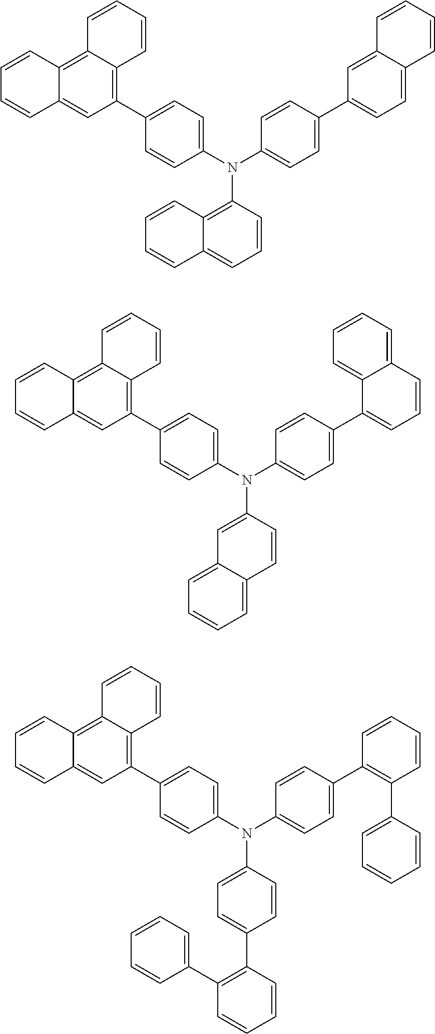 [SMILES: c1ccc2cc(-c3ccc(N(c4ccc(-c5cc6ccccc6c6ccccc56)cc4)c4cccc5ccccc45)cc3)ccc2c1 ; c1ccc2cc(N(c3ccc(-c4cccc5ccccc45)cc3)c3ccc(-c4cc5ccccc5c5ccccc45)cc3)ccc2c1 ; c1ccc(-c2ccccc2-c2ccc(N(c3ccc(-c4ccccc4-c4ccccc4)cc3)c3ccc(-c4cc5ccccc5c5ccccc45)cc3)cc2)cc1] 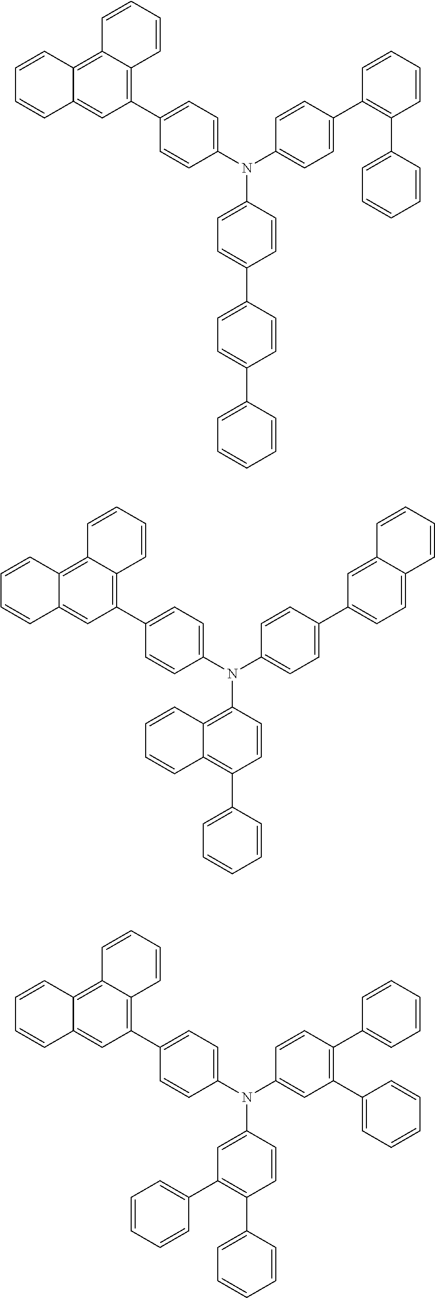 [SMILES: c1ccc(-c2ccc(-c3ccc(N(c4ccc(-c5ccccc5-c5ccccc5)cc4)c4ccc(-c5cc6ccccc6c6ccccc56)cc4)cc3)cc2)cc1 ; c1ccc(-c2ccc(N(c3ccc(-c4ccc5ccccc5c4)cc3)c3ccc(-c4cc5ccccc5c5ccccc45)cc3)c3ccccc23)cc1 ; c1ccc(-c2ccc(N(c3ccc(-c4cc5ccccc5c5ccccc45)cc3)c3ccc(-c4ccccc4)c(-c4ccccc4)c3)cc2-c2ccccc2)cc1] 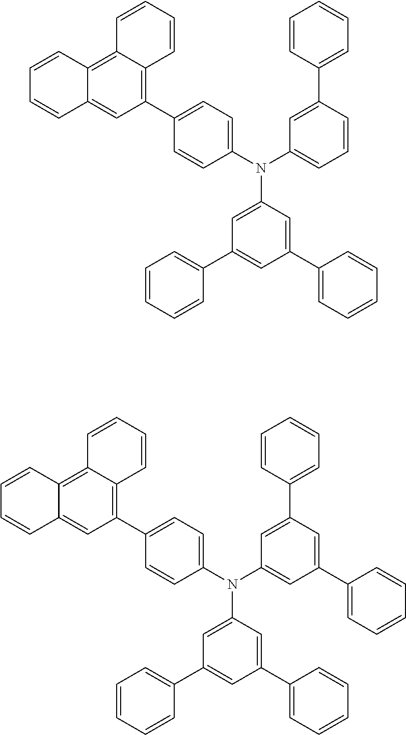 [SMILES: c1ccc(-c2cccc(N(c3ccc(-c4cc5ccccc5c5ccccc45)cc3)c3cc(-c4ccccc4)cc(-c4ccccc4)c3)c2)cc1 ; c1ccc(-c2cc(-c3ccccc3)cc(N(c3ccc(-c4cc5ccccc5c5ccccc45)cc3)c3cc(-c4ccccc4)cc(-c4ccccc4)c3)c2)cc1] 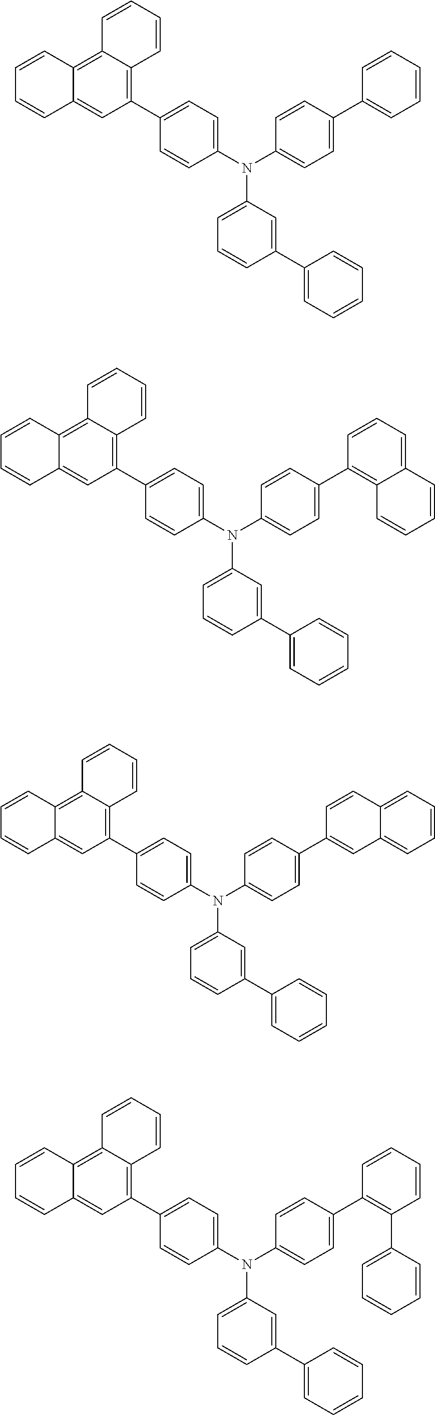 [SMILES: c1ccc(-c2ccc(N(c3ccc(-c4cc5ccccc5c5ccccc45)cc3)c3cccc(-c4ccccc4)c3)cc2)cc1 ; c1ccc(-c2cccc(N(c3ccc(-c4cccc5ccccc45)cc3)c3ccc(-c4cc5ccccc5c5ccccc45)cc3)c2)cc1 ; c1ccc(-c2cccc(N(c3ccc(-c4ccc5ccccc5c4)cc3)c3ccc(-c4cc5ccccc5c5ccccc45)cc3)c2)cc1 ; c1ccc(-c2cccc(N(c3ccc(-c4ccccc4-c4ccccc4)cc3)c3ccc(-c4cc5ccccc5c5ccccc45)cc3)c2)cc1] 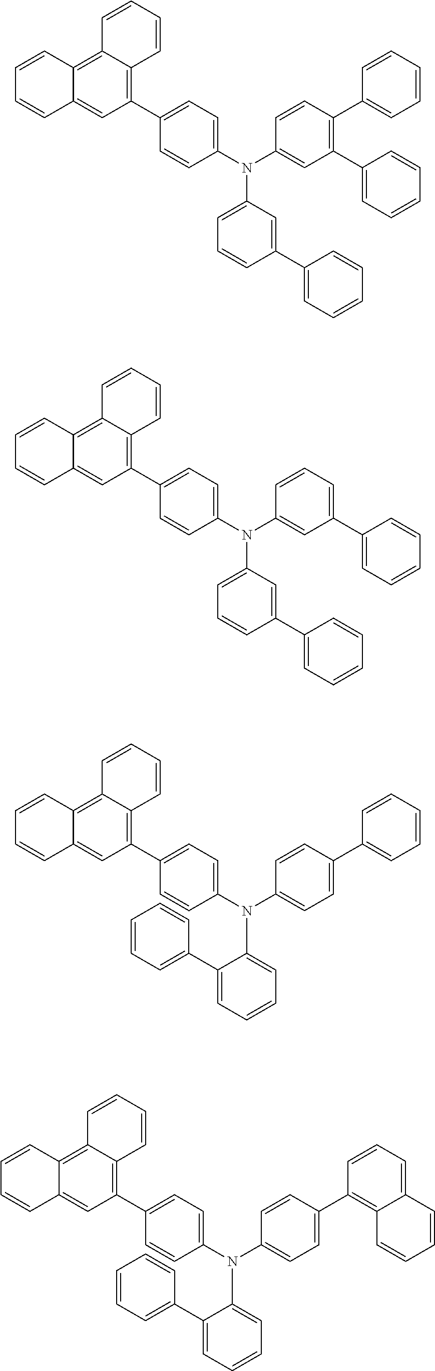 [SMILES: c1ccc(-c2cccc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cccc(-c4ccccc4)c3)c2)cc1 ; c1ccc(-c2ccc(N(c3ccc(-c4cc5ccccc5c5ccccc45)cc3)c3ccccc3-c3ccccc3)cc2)cc1 ; c1ccc(-c2ccccc2N(c2ccc(-c3cccc4ccccc34)cc2)c2ccc(-c3cc4ccccc4c4ccccc34)cc2)cc1] 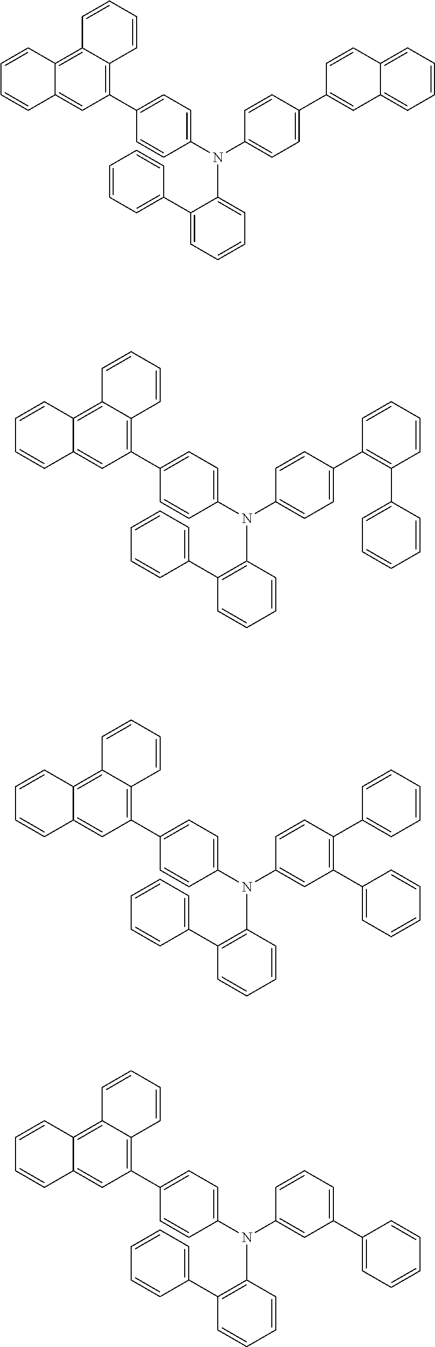 [SMILES: c1ccc(-c2ccccc2N(c2ccc(-c3ccc4ccccc4c3)cc2)c2ccc(-c3cc4ccccc4c4ccccc34)cc2)cc1 ; c1ccc(-c2ccccc2-c2ccc(N(c3ccc(-c4cc5ccccc5c5ccccc45)cc3)c3ccccc3-c3ccccc3)cc2)cc1 ; c1ccc(-c2ccc(N(c3ccc(-c4cc5ccccc5c5ccccc45)cc3)c3ccccc3-c3ccccc3)cc2-c2ccccc2)cc1 ; c1ccc(-c2cccc(N(c3ccc(-c4cc5ccccc5c5ccccc45)cc3)c3ccccc3-c3ccccc3)c2)cc1] 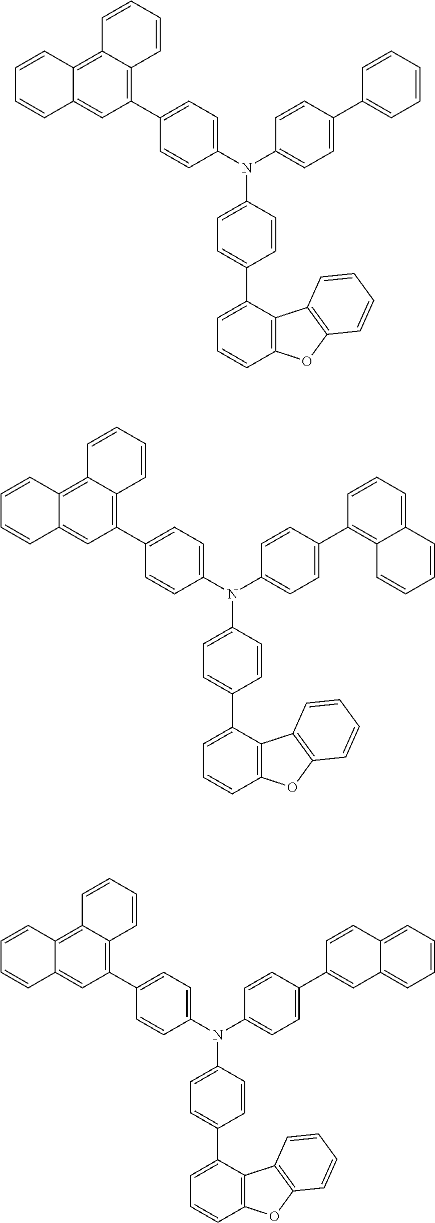 [SMILES: c1ccc(-c2ccc(N(c3ccc(-c4cc5ccccc5c5ccccc45)cc3)c3ccc(-c4cccc5oc6ccccc6c45)cc3)cc2)cc1 ; c1ccc2c(-c3ccc(N(c4ccc(-c5cc6ccccc6c6ccccc56)cc4)c4ccc(-c5cccc6oc7ccccc7c56)cc4)cc3)cccc2c1 ; c1ccc2cc(-c3ccc(N(c4ccc(-c5cc6ccccc6c6ccccc56)cc4)c4ccc(-c5cccc6oc7ccccc7c56)cc4)cc3)ccc2c1] 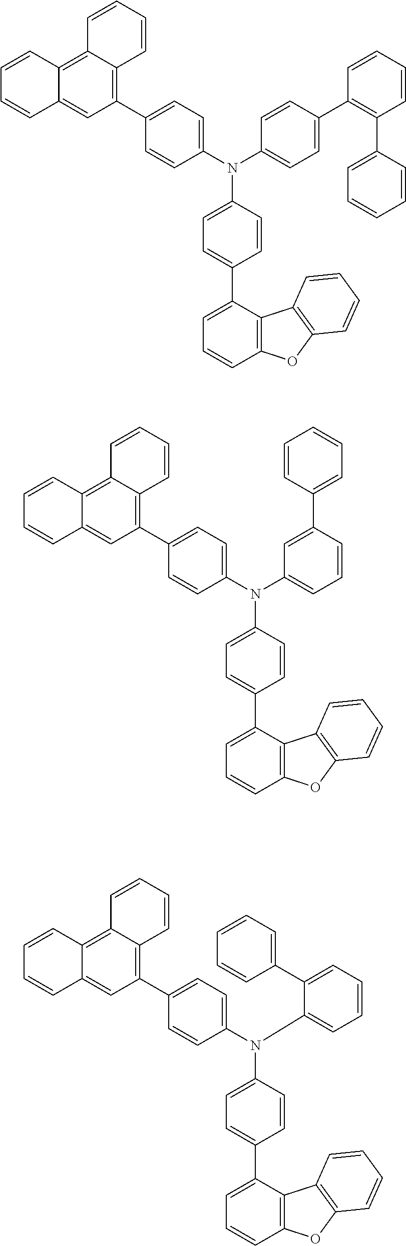 [SMILES: c1ccc(-c2ccccc2-c2ccc(N(c3ccc(-c4cc5ccccc5c5ccccc45)cc3)c3ccc(-c4cccc5oc6ccccc6c45)cc3)cc2)cc1 ; c1ccc(-c2cccc(N(c3ccc(-c4cc5ccccc5c5ccccc45)cc3)c3ccc(-c4cccc5oc6ccccc6c45)cc3)c2)cc1 ; c1ccc(-c2ccccc2N(c2ccc(-c3cc4ccccc4c4ccccc34)cc2)c2ccc(-c3cccc4oc5ccccc5c34)cc2)cc1] 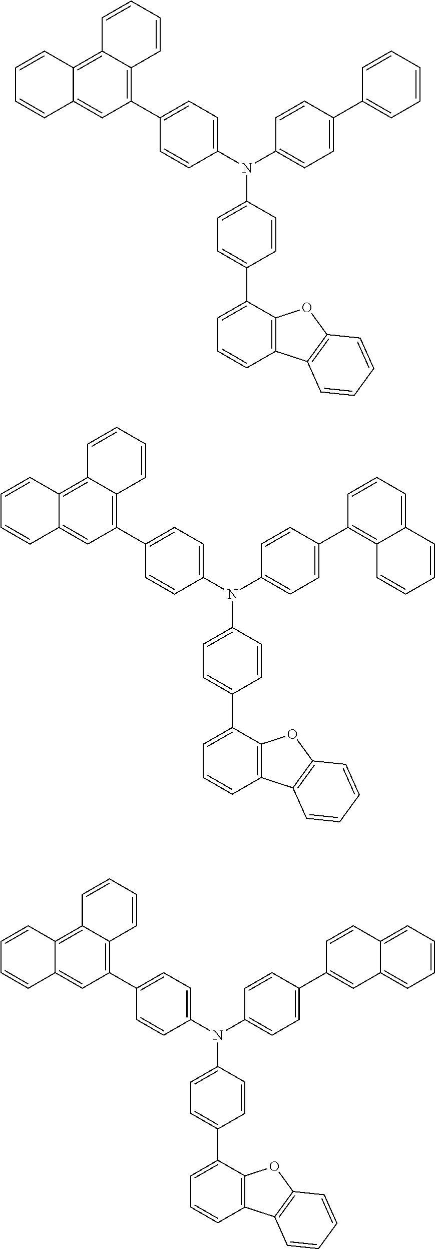 [SMILES: c1ccc(-c2ccc(N(c3ccc(-c4cc5ccccc5c5ccccc45)cc3)c3ccc(-c4cccc5c4oc4ccccc45)cc3)cc2)cc1 ; c1ccc2c(-c3ccc(N(c4ccc(-c5cc6ccccc6c6ccccc56)cc4)c4ccc(-c5cccc6c5oc5ccccc56)cc4)cc3)cccc2c1 ; c1ccc2cc(-c3ccc(N(c4ccc(-c5cc6ccccc6c6ccccc56)cc4)c4ccc(-c5cccc6c5oc5ccccc56)cc4)cc3)ccc2c1] 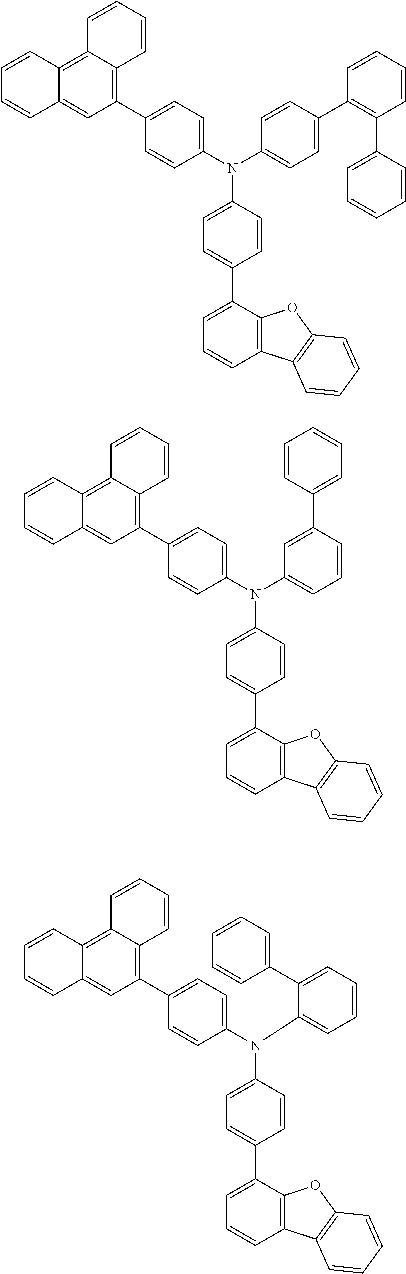 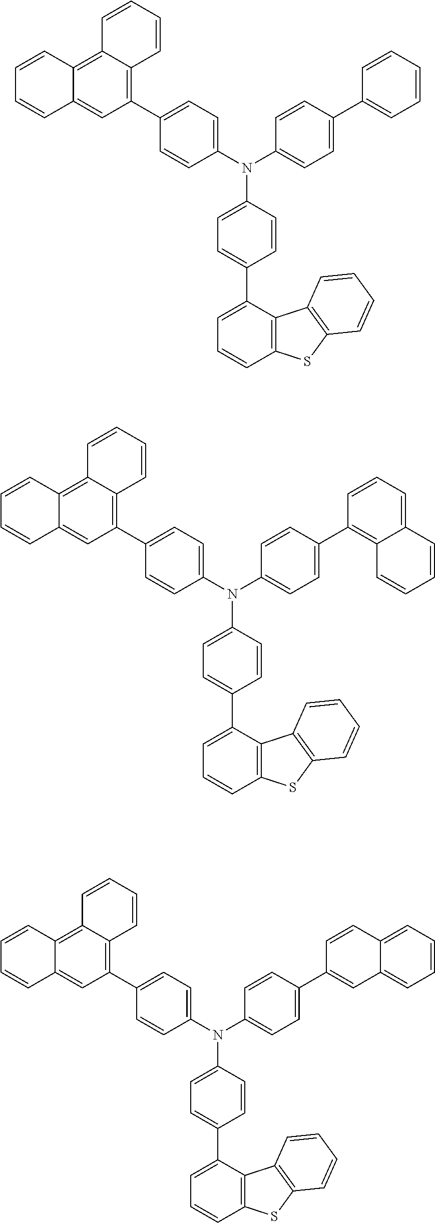 [SMILES: c1ccc(-c2ccc(N(c3ccc(-c4cc5ccccc5c5ccccc45)cc3)c3ccc(-c4cccc5sc6ccccc6c45)cc3)cc2)cc1 ; c1ccc2c(-c3ccc(N(c4ccc(-c5cc6ccccc6c6ccccc56)cc4)c4ccc(-c5cccc6sc7ccccc7c56)cc4)cc3)cccc2c1 ; c1ccc2cc(-c3ccc(N(c4ccc(-c5cc6ccccc6c6ccccc56)cc4)c4ccc(-c5cccc6sc7ccccc7c56)cc4)cc3)ccc2c1] 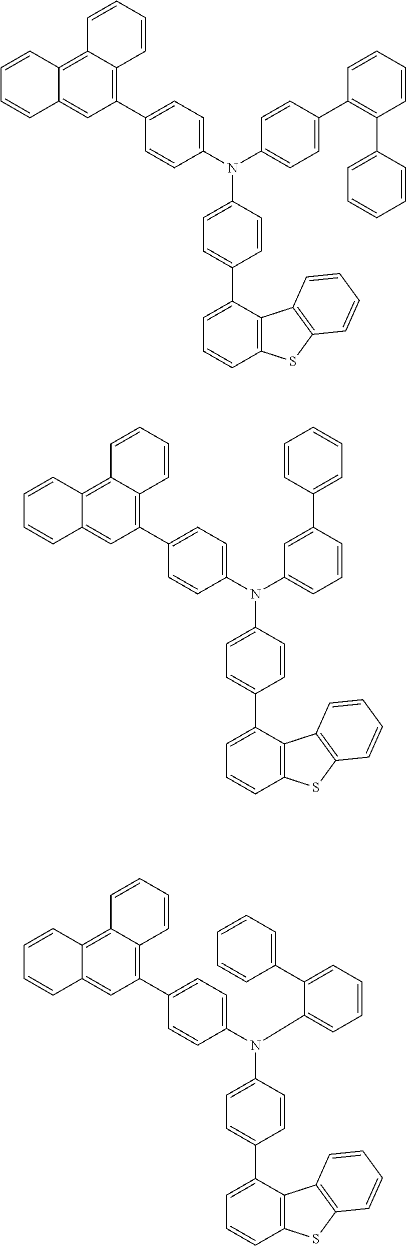 [SMILES: c1ccc(-c2ccccc2-c2ccc(N(c3ccc(-c4cc5ccccc5c5ccccc45)cc3)c3ccc(-c4cccc5sc6ccccc6c45)cc3)cc2)cc1 ; c1ccc(-c2cccc(N(c3ccc(-c4cc5ccccc5c5ccccc45)cc3)c3ccc(-c4cccc5sc6ccccc6c45)cc3)c2)cc1 ; c1ccc(-c2ccccc2N(c2ccc(-c3cc4ccccc4c4ccccc34)cc2)c2ccc(-c3cccc4sc5ccccc5c34)cc2)cc1] 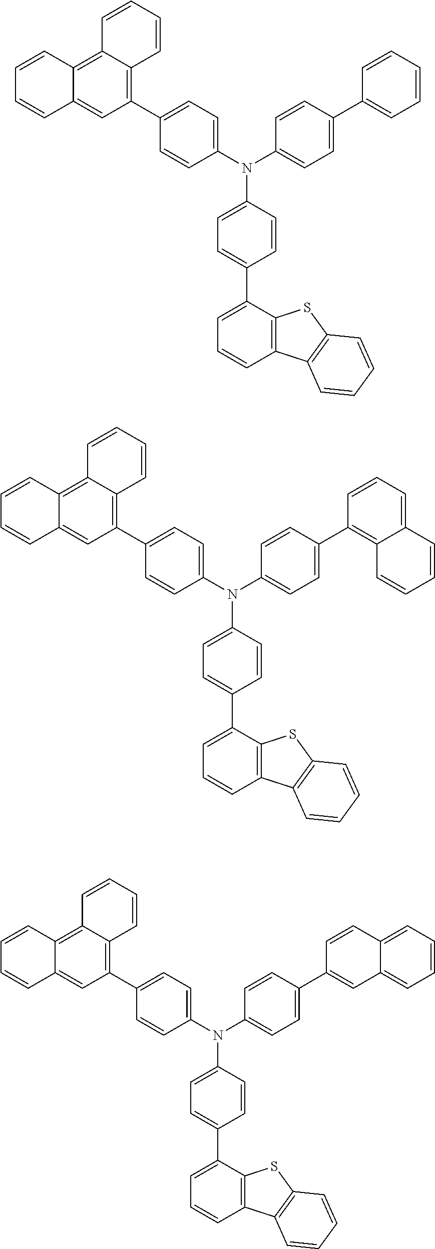 [SMILES: c1ccc(-c2ccc(N(c3ccc(-c4cc5ccccc5c5ccccc45)cc3)c3ccc(-c4cccc5c4sc4ccccc45)cc3)cc2)cc1 ; c1ccc2c(-c3ccc(N(c4ccc(-c5cc6ccccc6c6ccccc56)cc4)c4ccc(-c5cccc6c5sc5ccccc56)cc4)cc3)cccc2c1 ; c1ccc2cc(-c3ccc(N(c4ccc(-c5cc6ccccc6c6ccccc56)cc4)c4ccc(-c5cccc6c5sc5ccccc56)cc4)cc3)ccc2c1] 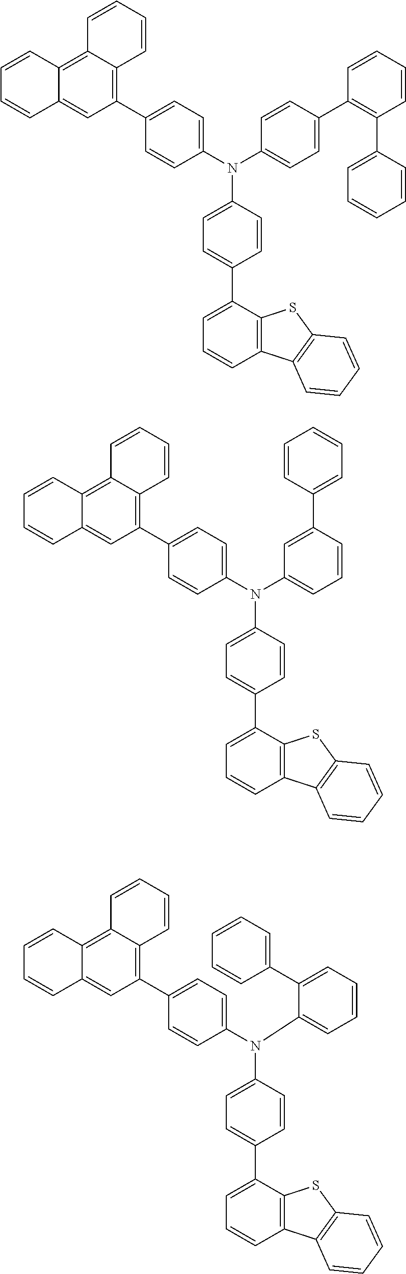 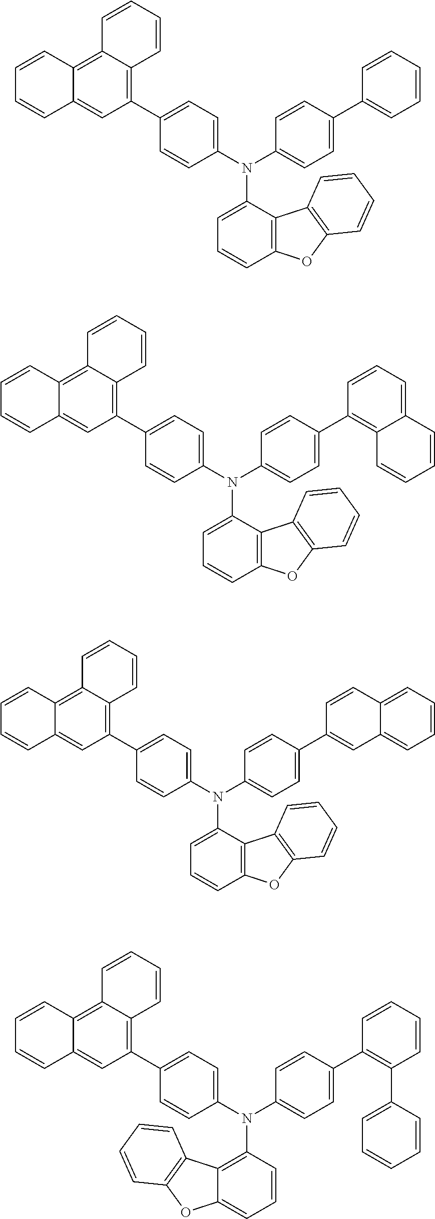 [SMILES: c1ccc(-c2ccc(N(c3ccc(-c4cc5ccccc5c5ccccc45)cc3)c3cccc4oc5ccccc5c34)cc2)cc1 ; c1ccc2c(-c3ccc(N(c4ccc(-c5cc6ccccc6c6ccccc56)cc4)c4cccc5oc6ccccc6c45)cc3)cccc2c1 ; c1ccc2cc(-c3ccc(N(c4ccc(-c5cc6ccccc6c6ccccc56)cc4)c4cccc5oc6ccccc6c45)cc3)ccc2c1 ; c1ccc(-c2ccccc2-c2ccc(N(c3ccc(-c4cc5ccccc5c5ccccc45)cc3)c3cccc4oc5ccccc5c34)cc2)cc1] 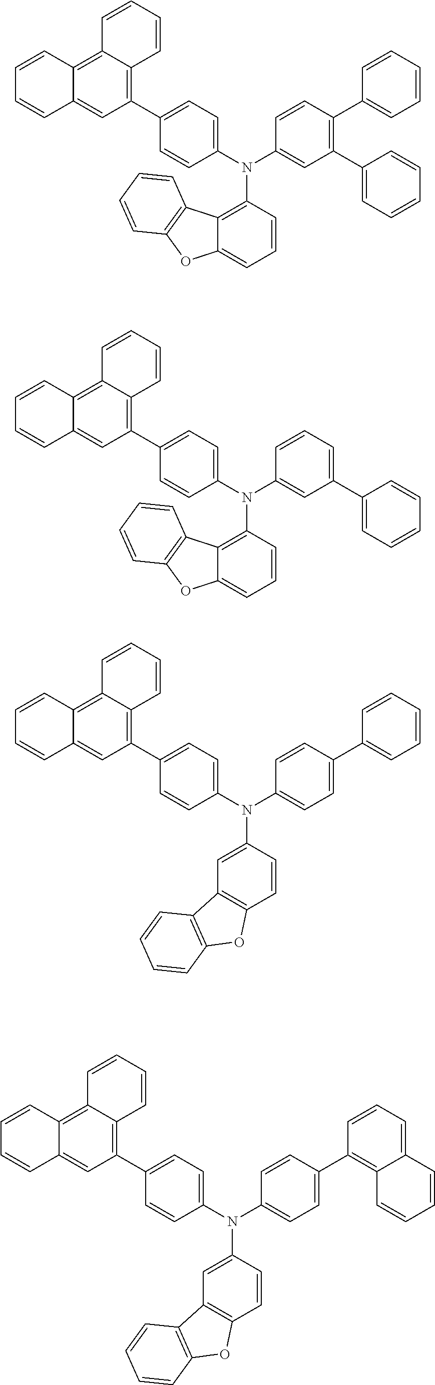 [SMILES: c1ccc(-c2ccc(N(c3ccc(-c4cc5ccccc5c5ccccc45)cc3)c3cccc4oc5ccccc5c34)cc2-c2ccccc2)cc1 ; c1ccc(-c2cccc(N(c3ccc(-c4cc5ccccc5c5ccccc45)cc3)c3cccc4oc5ccccc5c34)c2)cc1 ; c1ccc(-c2ccc(N(c3ccc(-c4cc5ccccc5c5ccccc45)cc3)c3ccc4oc5ccccc5c4c3)cc2)cc1 ; c1ccc2c(-c3ccc(N(c4ccc(-c5cc6ccccc6c6ccccc56)cc4)c4ccc5oc6ccccc6c5c4)cc3)cccc2c1] 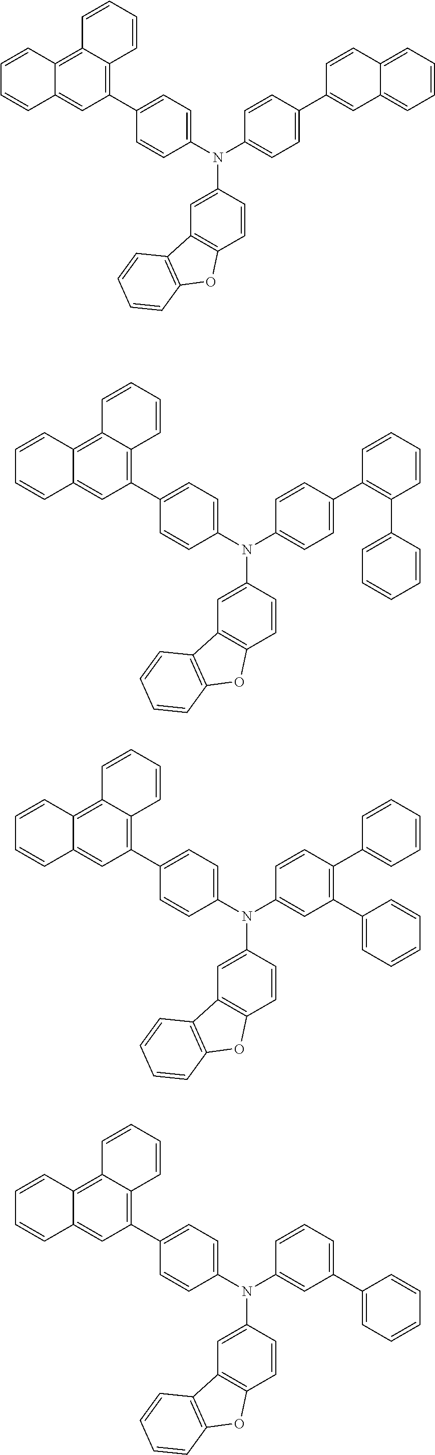 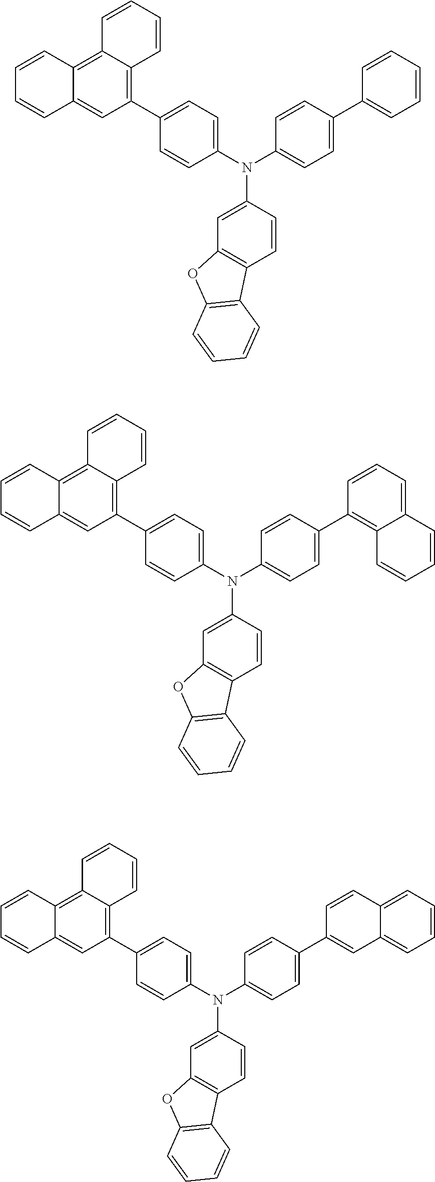 [SMILES: c1ccc(-c2ccc(N(c3ccc(-c4cc5ccccc5c5ccccc45)cc3)c3ccc4c(c3)oc3ccccc34)cc2)cc1 ; c1ccc2c(-c3ccc(N(c4ccc(-c5cc6ccccc6c6ccccc56)cc4)c4ccc5c(c4)oc4ccccc45)cc3)cccc2c1 ; c1ccc2cc(-c3ccc(N(c4ccc(-c5cc6ccccc6c6ccccc56)cc4)c4ccc5c(c4)oc4ccccc45)cc3)ccc2c1] 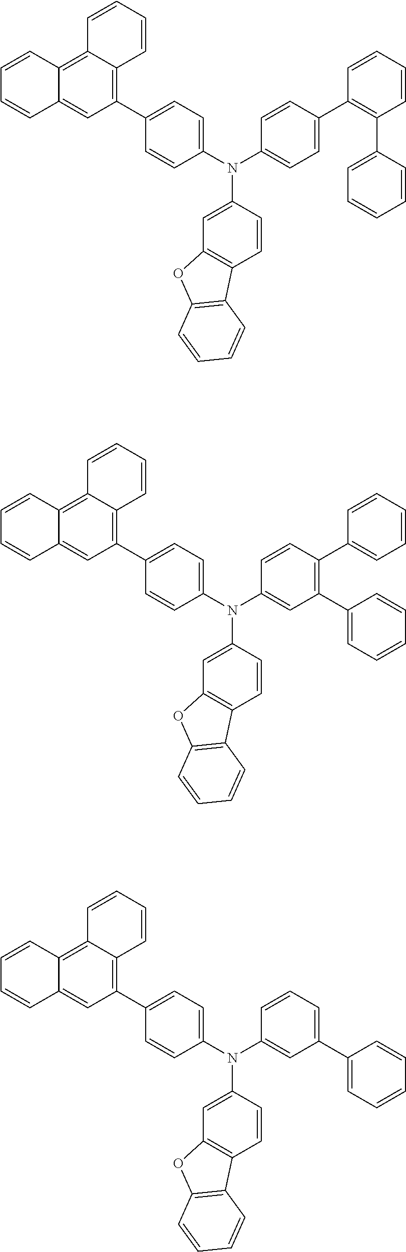 [SMILES: c1ccc(-c2ccccc2-c2ccc(N(c3ccc(-c4cc5ccccc5c5ccccc45)cc3)c3ccc4c(c3)oc3ccccc34)cc2)cc1 ; c1ccc(-c2ccc(N(c3ccc(-c4cc5ccccc5c5ccccc45)cc3)c3ccc4c(c3)oc3ccccc34)cc2-c2ccccc2)cc1 ; c1ccc(-c2cccc(N(c3ccc(-c4cc5ccccc5c5ccccc45)cc3)c3ccc4c(c3)oc3ccccc34)c2)cc1] 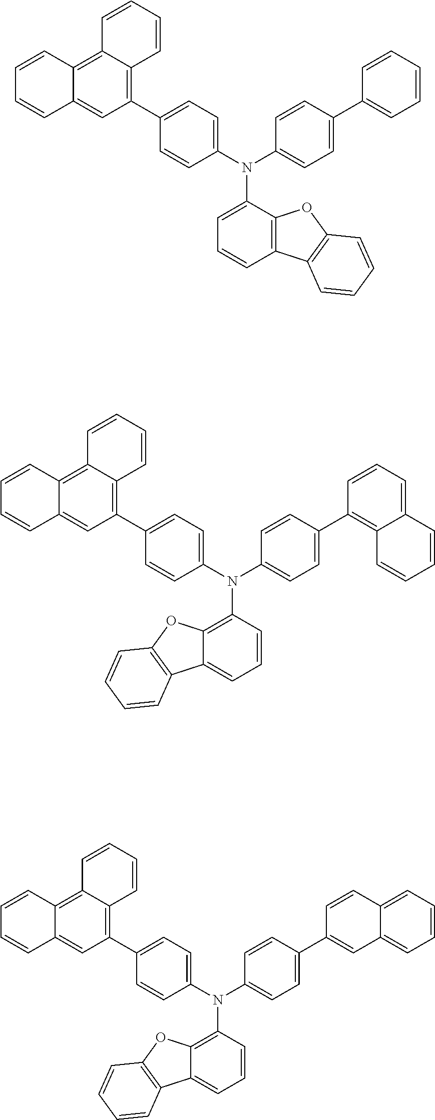 [SMILES: c1ccc(-c2ccc(N(c3ccc(-c4cc5ccccc5c5ccccc45)cc3)c3cccc4c3oc3ccccc34)cc2)cc1 ; c1ccc2c(-c3ccc(N(c4ccc(-c5cc6ccccc6c6ccccc56)cc4)c4cccc5c4oc4ccccc45)cc3)cccc2c1 ; c1ccc2cc(-c3ccc(N(c4ccc(-c5cc6ccccc6c6ccccc56)cc4)c4cccc5c4oc4ccccc45)cc3)ccc2c1] 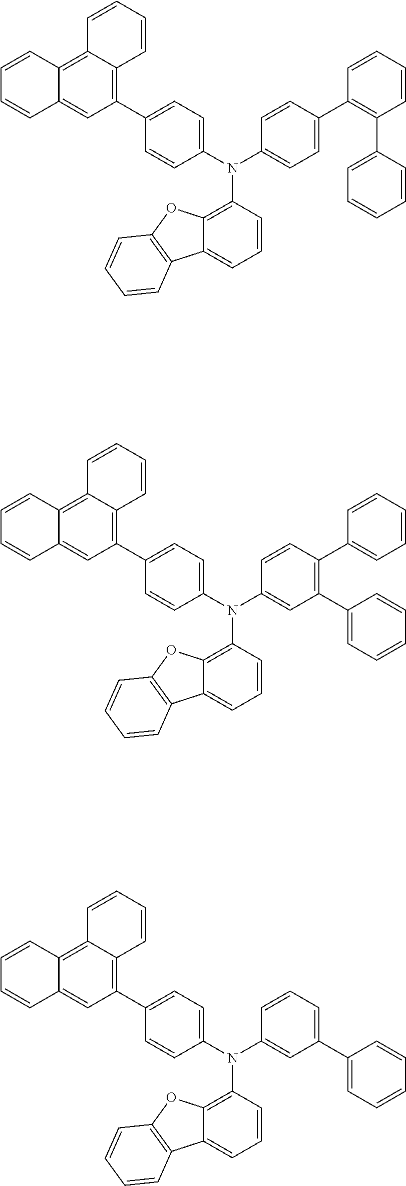 [SMILES: c1ccc(-c2ccccc2-c2ccc(N(c3ccc(-c4cc5ccccc5c5ccccc45)cc3)c3cccc4c3oc3ccccc34)cc2)cc1 ; c1ccc(-c2ccc(N(c3ccc(-c4cc5ccccc5c5ccccc45)cc3)c3cccc4c3oc3ccccc34)cc2-c2ccccc2)cc1 ; c1ccc(-c2cccc(N(c3ccc(-c4cc5ccccc5c5ccccc45)cc3)c3cccc4c3oc3ccccc34)c2)cc1] 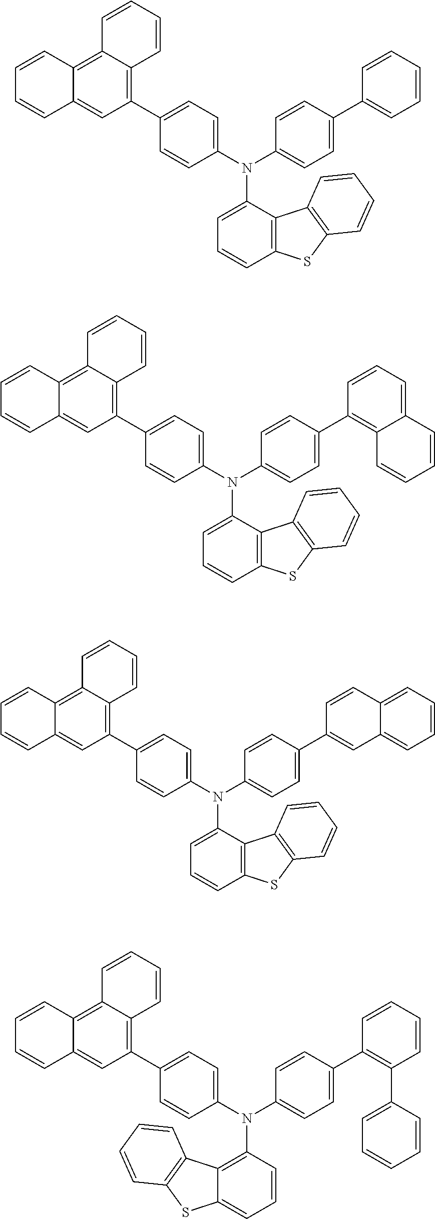 [SMILES: c1ccc(-c2ccc(N(c3ccc(-c4cc5ccccc5c5ccccc45)cc3)c3cccc4sc5ccccc5c34)cc2)cc1 ; c1ccc2c(-c3ccc(N(c4ccc(-c5cc6ccccc6c6ccccc56)cc4)c4cccc5sc6ccccc6c45)cc3)cccc2c1 ; c1ccc2cc(-c3ccc(N(c4ccc(-c5cc6ccccc6c6ccccc56)cc4)c4cccc5sc6ccccc6c45)cc3)ccc2c1 ; c1ccc(-c2ccccc2-c2ccc(N(c3ccc(-c4cc5ccccc5c5ccccc45)cc3)c3cccc4sc5ccccc5c34)cc2)cc1] 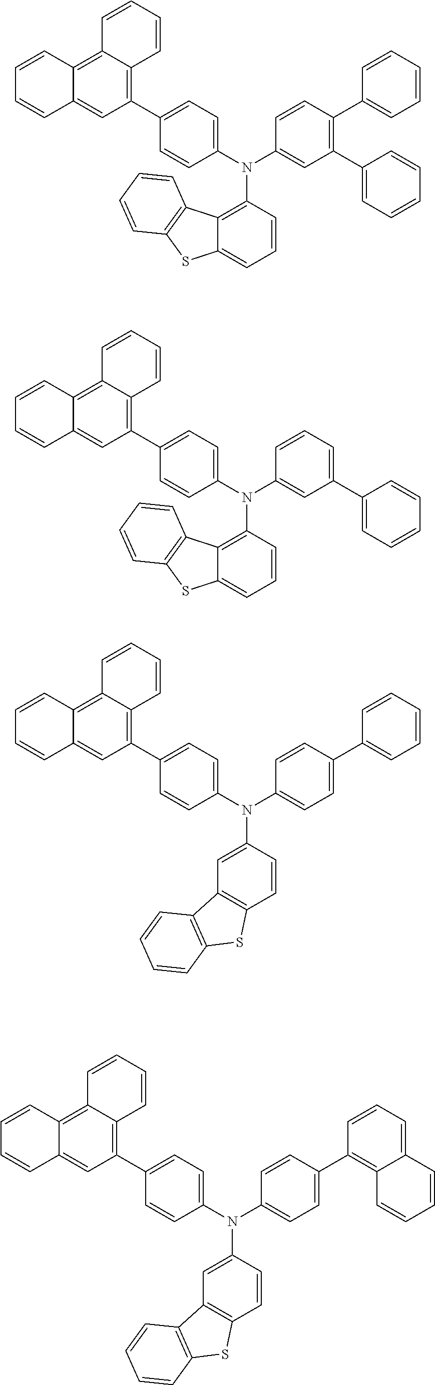 [SMILES: c1ccc(-c2ccc(N(c3ccc(-c4cc5ccccc5c5ccccc45)cc3)c3cccc4sc5ccccc5c34)cc2-c2ccccc2)cc1 ; c1ccc(-c2cccc(N(c3ccc(-c4cc5ccccc5c5ccccc45)cc3)c3cccc4sc5ccccc5c34)c2)cc1 ; c1ccc(-c2ccc(N(c3ccc(-c4cc5ccccc5c5ccccc45)cc3)c3ccc4sc5ccccc5c4c3)cc2)cc1 ; c1ccc2c(-c3ccc(N(c4ccc(-c5cc6ccccc6c6ccccc56)cc4)c4ccc5sc6ccccc6c5c4)cc3)cccc2c1] 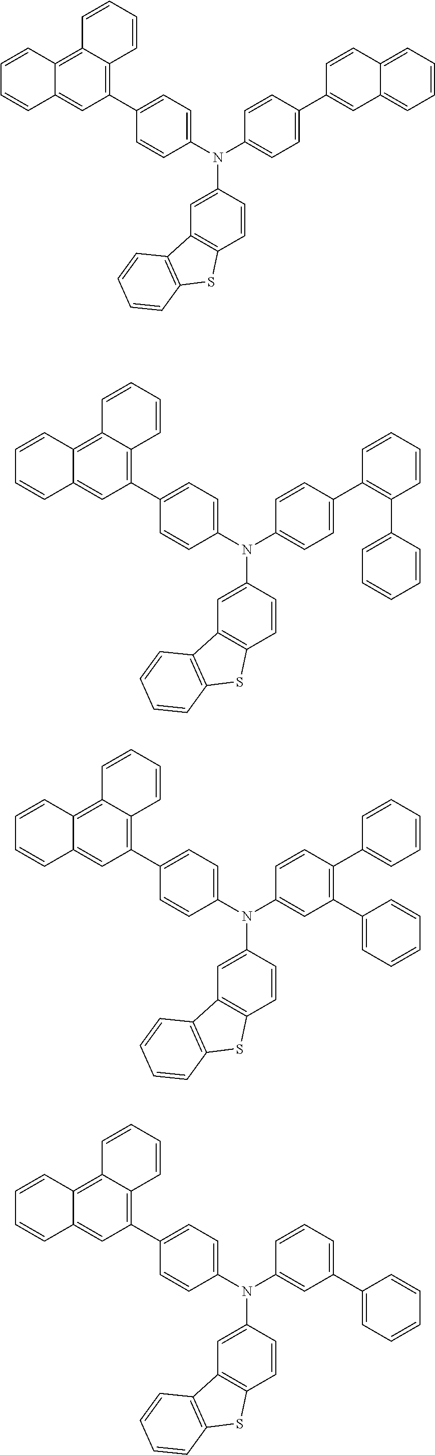 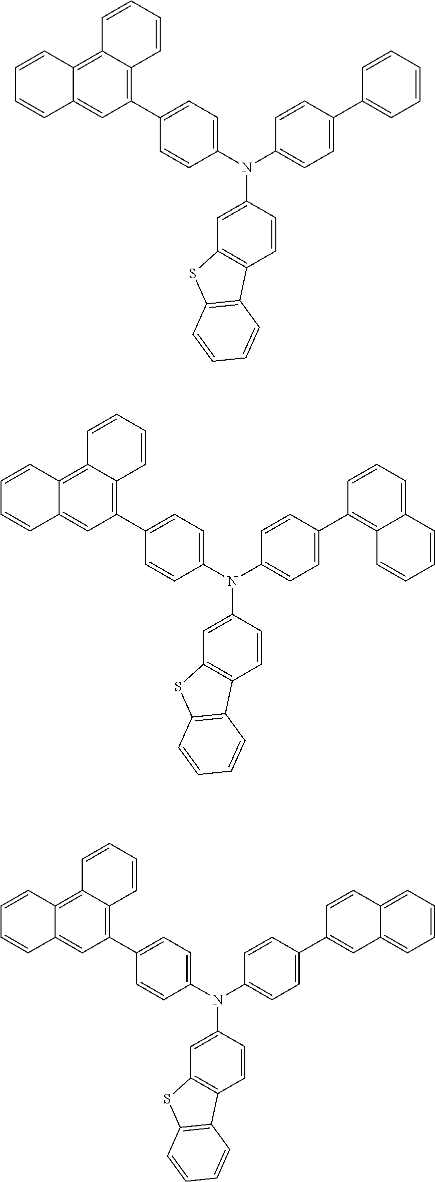 [SMILES: c1ccc(-c2ccc(N(c3ccc(-c4cc5ccccc5c5ccccc45)cc3)c3ccc4c(c3)sc3ccccc34)cc2)cc1 ; c1ccc2c(-c3ccc(N(c4ccc(-c5cc6ccccc6c6ccccc56)cc4)c4ccc5c(c4)sc4ccccc45)cc3)cccc2c1 ; c1ccc2cc(-c3ccc(N(c4ccc(-c5cc6ccccc6c6ccccc56)cc4)c4ccc5c(c4)sc4ccccc45)cc3)ccc2c1] 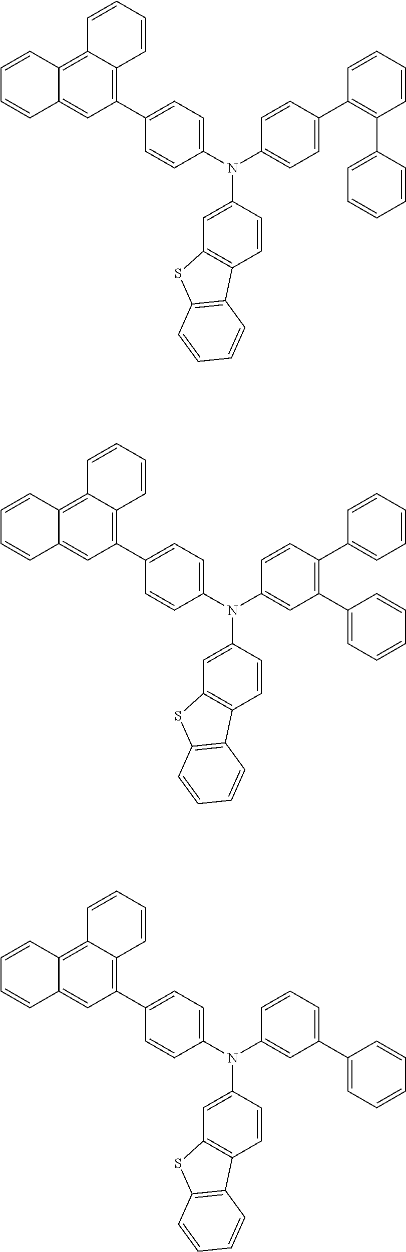 [SMILES: c1ccc(-c2ccccc2-c2ccc(N(c3ccc(-c4cc5ccccc5c5ccccc45)cc3)c3ccc4c(c3)sc3ccccc34)cc2)cc1 ; c1ccc(-c2ccc(N(c3ccc(-c4cc5ccccc5c5ccccc45)cc3)c3ccc4c(c3)sc3ccccc34)cc2-c2ccccc2)cc1 ; c1ccc(-c2cccc(N(c3ccc(-c4cc5ccccc5c5ccccc45)cc3)c3ccc4c(c3)sc3ccccc34)c2)cc1] 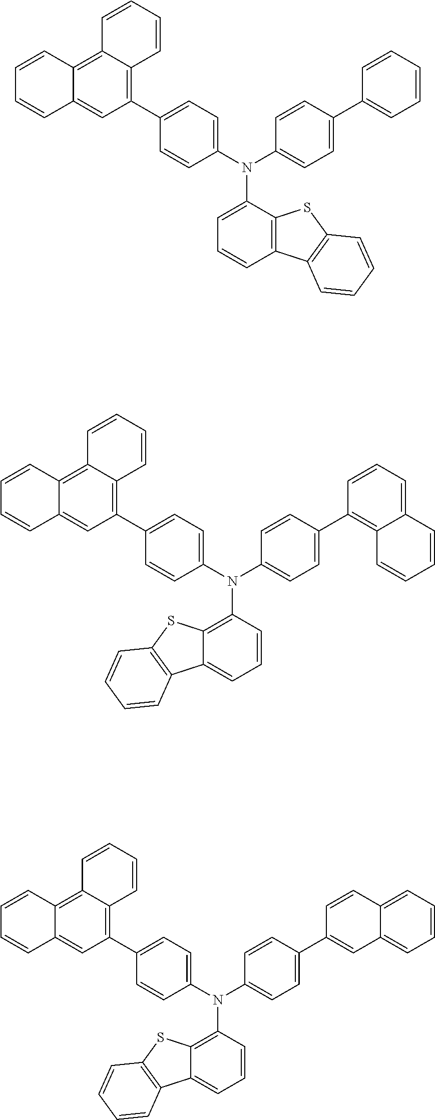 [SMILES: c1ccc(-c2ccc(N(c3ccc(-c4cc5ccccc5c5ccccc45)cc3)c3cccc4c3sc3ccccc34)cc2)cc1 ; c1ccc2c(-c3ccc(N(c4ccc(-c5cc6ccccc6c6ccccc56)cc4)c4cccc5c4sc4ccccc45)cc3)cccc2c1 ; c1ccc2cc(-c3ccc(N(c4ccc(-c5cc6ccccc6c6ccccc56)cc4)c4cccc5c4sc4ccccc45)cc3)ccc2c1] 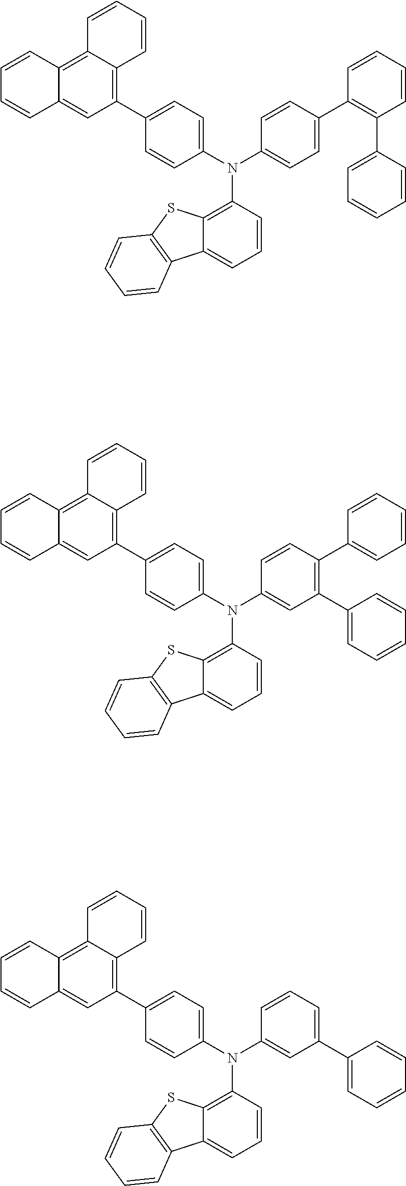 [SMILES: c1ccc(-c2ccccc2-c2ccc(N(c3ccc(-c4cc5ccccc5c5ccccc45)cc3)c3cccc4c3sc3ccccc34)cc2)cc1 ; c1ccc(-c2ccc(N(c3ccc(-c4cc5ccccc5c5ccccc45)cc3)c3cccc4c3sc3ccccc34)cc2-c2ccccc2)cc1 ; c1ccc(-c2cccc(N(c3ccc(-c4cc5ccccc5c5ccccc45)cc3)c3cccc4c3sc3ccccc34)c2)cc1] 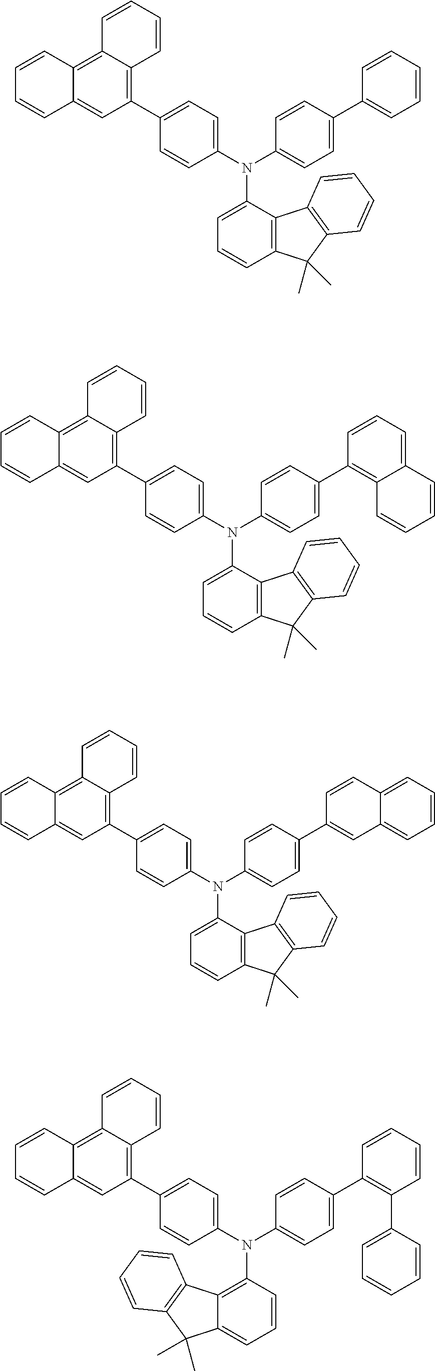 [SMILES: CC1(C)c2ccccc2-c2c(N(c3ccc(-c4ccccc4)cc3)c3ccc(-c4cc5ccccc5c5ccccc45)cc3)cccc21 ; CC1(C)c2ccccc2-c2c(N(c3ccc(-c4cccc5ccccc45)cc3)c3ccc(-c4cc5ccccc5c5ccccc45)cc3)cccc21 ; CC1(C)c2ccccc2-c2c(N(c3ccc(-c4ccc5ccccc5c4)cc3)c3ccc(-c4cc5ccccc5c5ccccc45)cc3)cccc21 ; CC1(C)c2ccccc2-c2c(N(c3ccc(-c4ccccc4-c4ccccc4)cc3)c3ccc(-c4cc5ccccc5c5ccccc45)cc3)cccc21] 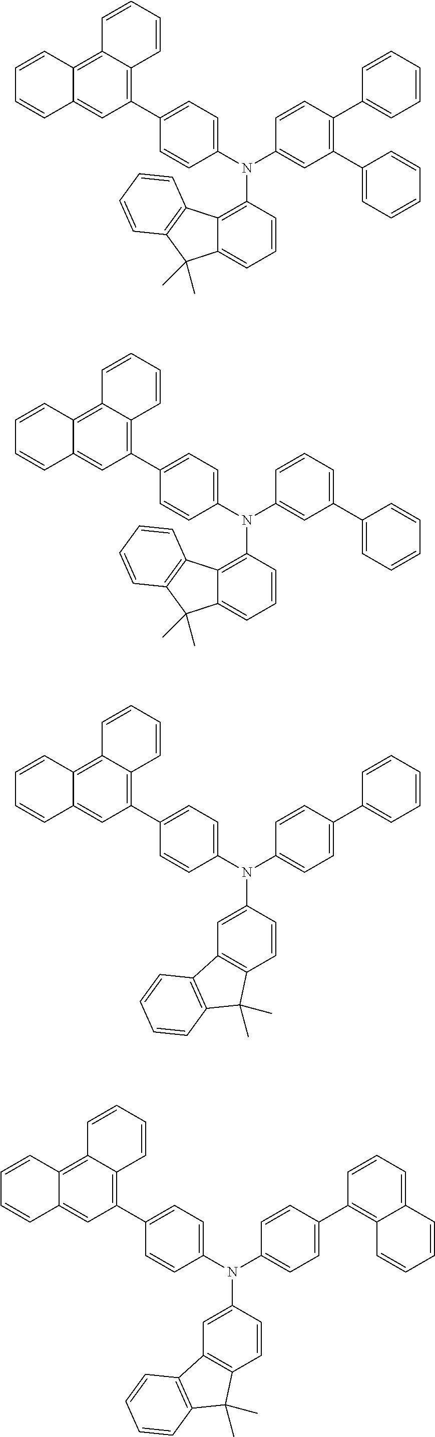 [SMILES: CC1(C)c2ccccc2-c2c(N(c3ccc(-c4cc5ccccc5c5ccccc45)cc3)c3ccc(-c4ccccc4)c(-c4ccccc4)c3)cccc21 ; CC1(C)c2ccccc2-c2c(N(c3ccc(-c4cc5ccccc5c5ccccc45)cc3)c3cccc(-c4ccccc4)c3)cccc21 ; CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2cc(N(c3ccc(-c4cccc5ccccc45)cc3)c3ccc(-c4cc5ccccc5c5ccccc45)cc3)ccc21] 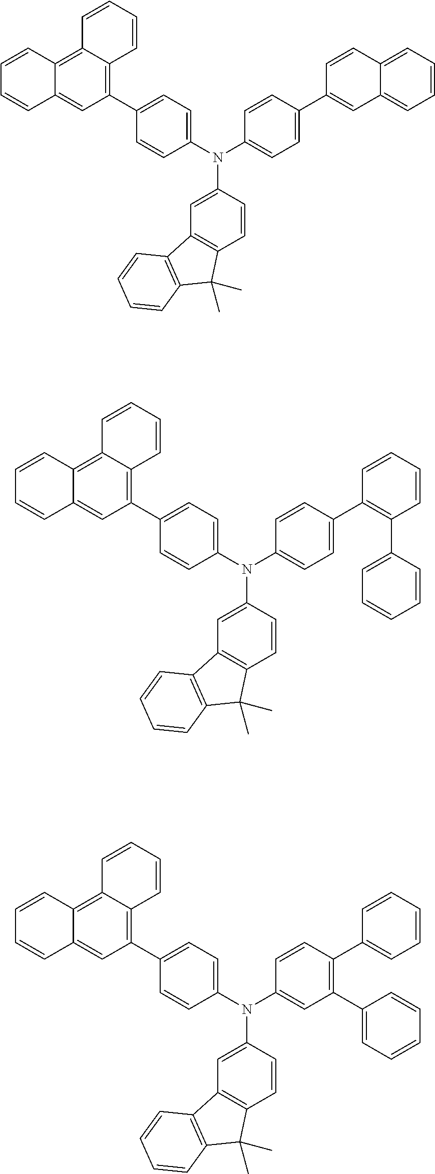 [SMILES: CC1(C)c2ccccc2-c2cc(N(c3ccc(-c4ccc5ccccc5c4)cc3)c3ccc(-c4cc5ccccc5c5ccccc45)cc3)ccc21 ; CC1(C)c2ccccc2-c2cc(N(c3ccc(-c4ccccc4-c4ccccc4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3ccc(-c4ccccc4)c(-c4ccccc4)c3)ccc21] 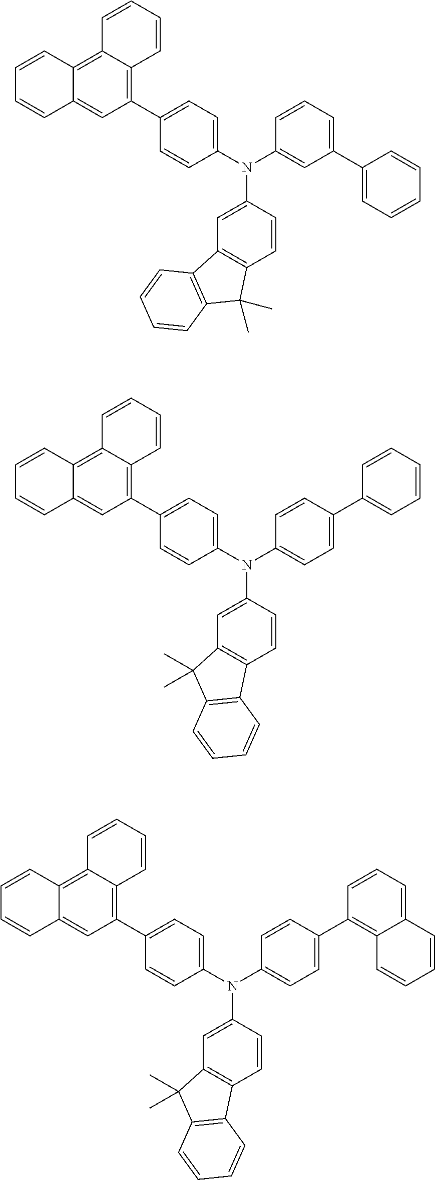 [SMILES: CC1(C)c2ccccc2-c2cc(N(c3ccc(-c4cc5ccccc5c5ccccc45)cc3)c3cccc(-c4ccccc4)c3)ccc21 ; CC1(C)c2ccccc2-c2ccc(N(c3ccc(-c4ccccc4)cc3)c3ccc(-c4cc5ccccc5c5ccccc45)cc3)cc21 ; CC1(C)c2ccccc2-c2ccc(N(c3ccc(-c4cccc5ccccc45)cc3)c3ccc(-c4cc5ccccc5c5ccccc45)cc3)cc21] 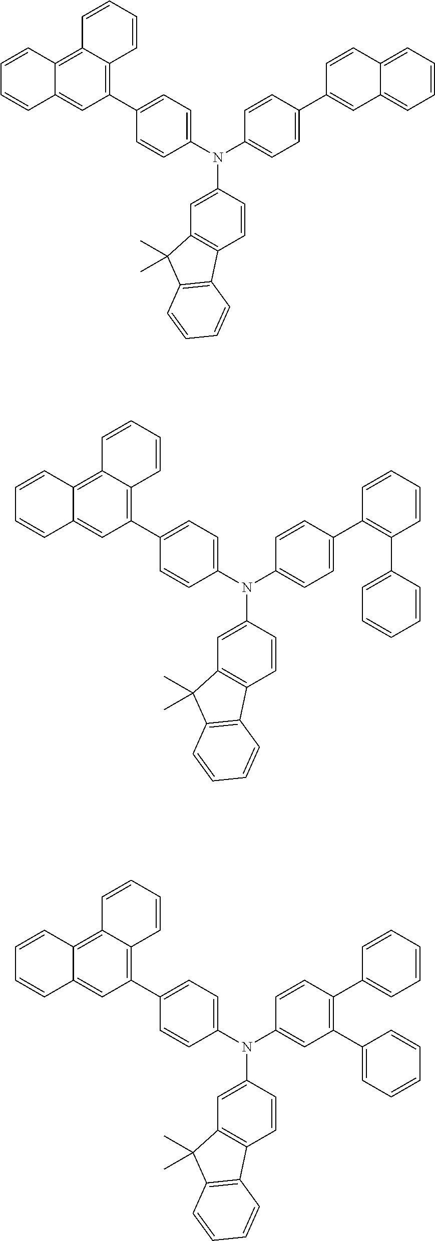 [SMILES: CC1(C)c2ccccc2-c2ccc(N(c3ccc(-c4ccc5ccccc5c4)cc3)c3ccc(-c4cc5ccccc5c5ccccc45)cc3)cc21 ; CC1(C)c2ccccc2-c2ccc(N(c3ccc(-c4ccccc4-c4ccccc4)cc3)c3ccc(-c4cc5ccccc5c5ccccc45)cc3)cc21 ; CC1(C)c2ccccc2-c2ccc(N(c3ccc(-c4cc5ccccc5c5ccccc45)cc3)c3ccc(-c4ccccc4)c(-c4ccccc4)c3)cc21] 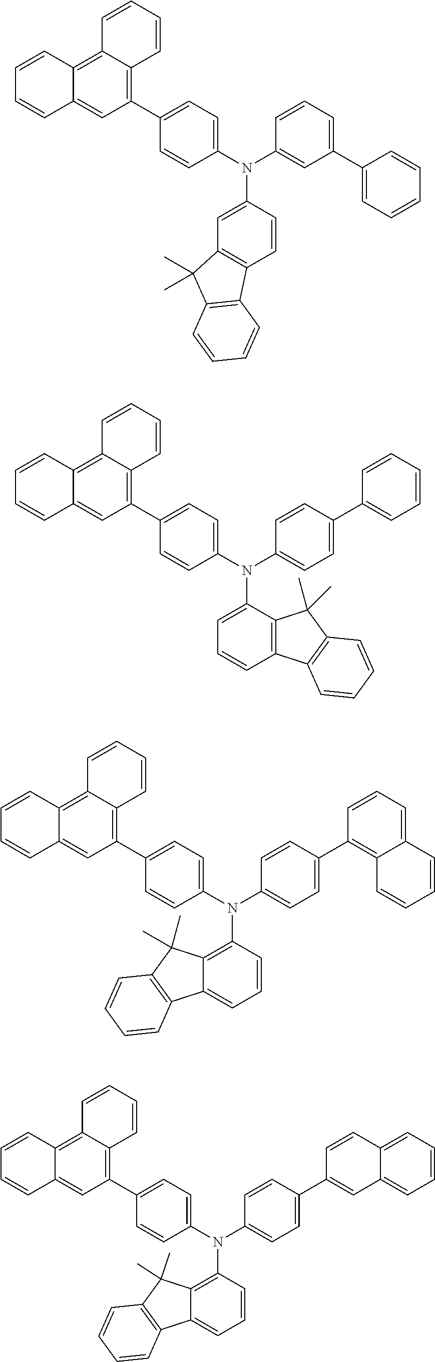 [SMILES: CC1(C)c2ccccc2-c2ccc(N(c3ccc(-c4cc5ccccc5c5ccccc45)cc3)c3cccc(-c4ccccc4)c3)cc21 ; CC1(C)c2ccccc2-c2cccc(N(c3ccc(-c4ccccc4)cc3)c3ccc(-c4cc5ccccc5c5ccccc45)cc3)c21 ; CC1(C)c2ccccc2-c2cccc(N(c3ccc(-c4cccc5ccccc45)cc3)c3ccc(-c4cc5ccccc5c5ccccc45)cc3)c21 ; CC1(C)c2ccccc2-c2cccc(N(c3ccc(-c4ccc5ccccc5c4)cc3)c3ccc(-c4cc5ccccc5c5ccccc45)cc3)c21] 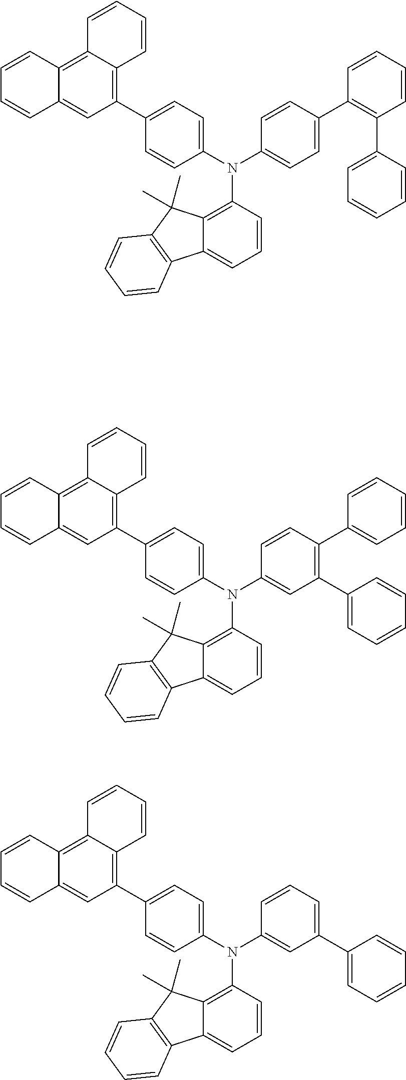 [SMILES: CC1(C)c2ccccc2-c2cccc(N(c3ccc(-c4ccccc4-c4ccccc4)cc3)c3ccc(-c4cc5ccccc5c5ccccc45)cc3)c21 ; CC1(C)c2ccccc2-c2cccc(N(c3ccc(-c4cc5ccccc5c5ccccc45)cc3)c3ccc(-c4ccccc4)c(-c4ccccc4)c3)c21 ; CC1(C)c2ccccc2-c2cccc(N(c3ccc(-c4cc5ccccc5c5ccccc45)cc3)c3cccc(-c4ccccc4)c3)c21] 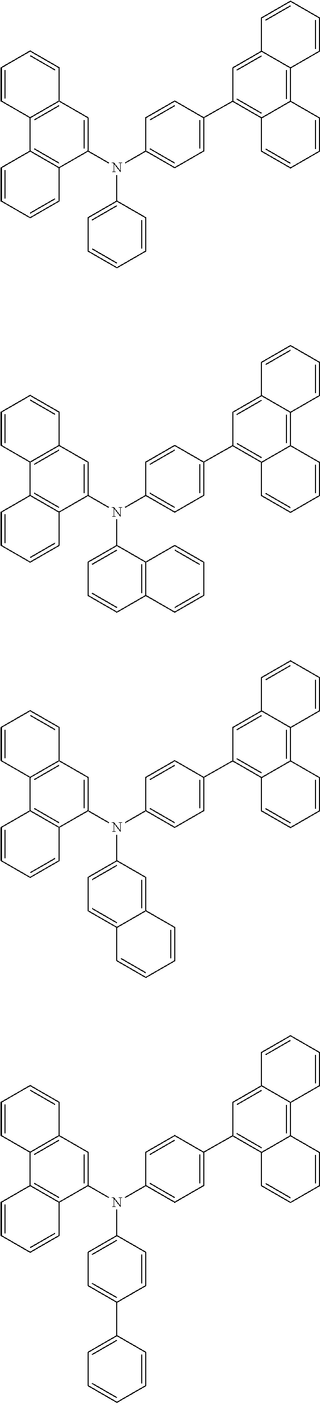 [SMILES: c1ccc(N(c2ccc(-c3cc4ccccc4c4ccccc34)cc2)c2cc3ccccc3c3ccccc23)cc1 ; c1ccc2c(N(c3ccc(-c4cc5ccccc5c5ccccc45)cc3)c3cc4ccccc4c4ccccc34)cccc2c1 ; c1ccc2cc(N(c3ccc(-c4cc5ccccc5c5ccccc45)cc3)c3cc4ccccc4c4ccccc34)ccc2c1 ; c1ccc(-c2ccc(N(c3ccc(-c4cc5ccccc5c5ccccc45)cc3)c3cc4ccccc4c4ccccc34)cc2)cc1] 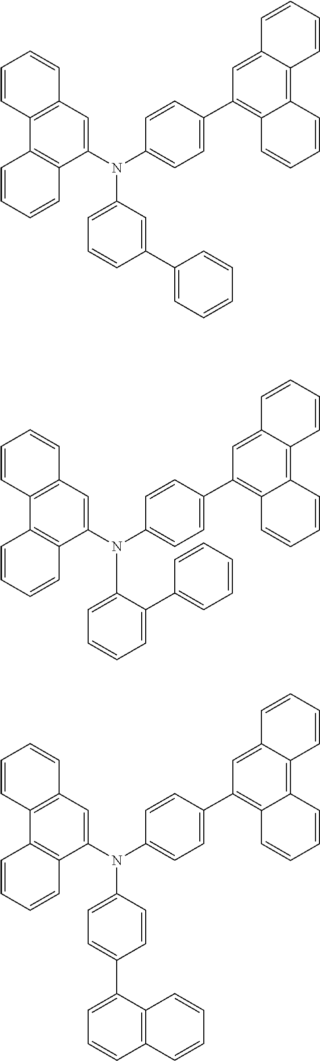 [SMILES: c1ccc(-c2cccc(N(c3ccc(-c4cc5ccccc5c5ccccc45)cc3)c3cc4ccccc4c4ccccc34)c2)cc1 ; c1ccc(-c2ccccc2N(c2ccc(-c3cc4ccccc4c4ccccc34)cc2)c2cc3ccccc3c3ccccc23)cc1 ; c1ccc2c(-c3ccc(N(c4ccc(-c5cc6ccccc6c6ccccc56)cc4)c4cc5ccccc5c5ccccc45)cc3)cccc2c1] 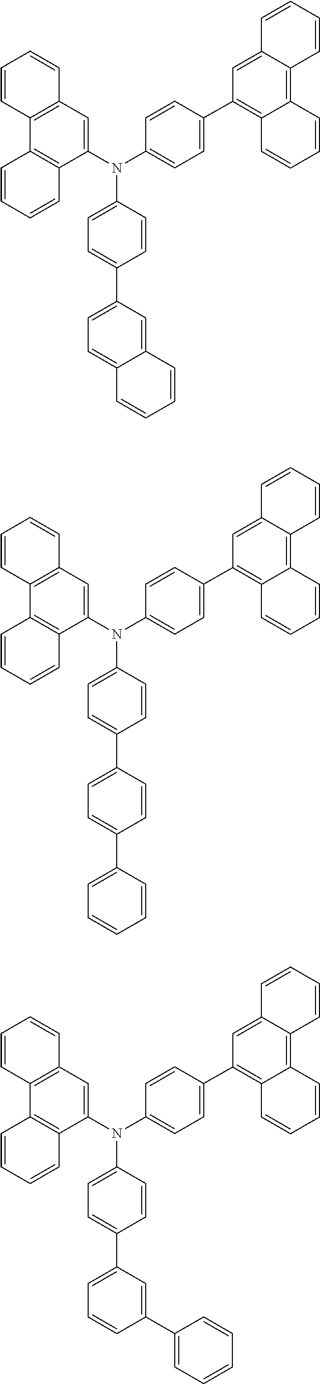 [SMILES: c1ccc2cc(-c3ccc(N(c4ccc(-c5cc6ccccc6c6ccccc56)cc4)c4cc5ccccc5c5ccccc45)cc3)ccc2c1 ; c1ccc(-c2ccc(-c3ccc(N(c4ccc(-c5cc6ccccc6c6ccccc56)cc4)c4cc5ccccc5c5ccccc45)cc3)cc2)cc1 ; c1ccc(-c2cccc(-c3ccc(N(c4ccc(-c5cc6ccccc6c6ccccc56)cc4)c4cc5ccccc5c5ccccc45)cc3)c2)cc1] 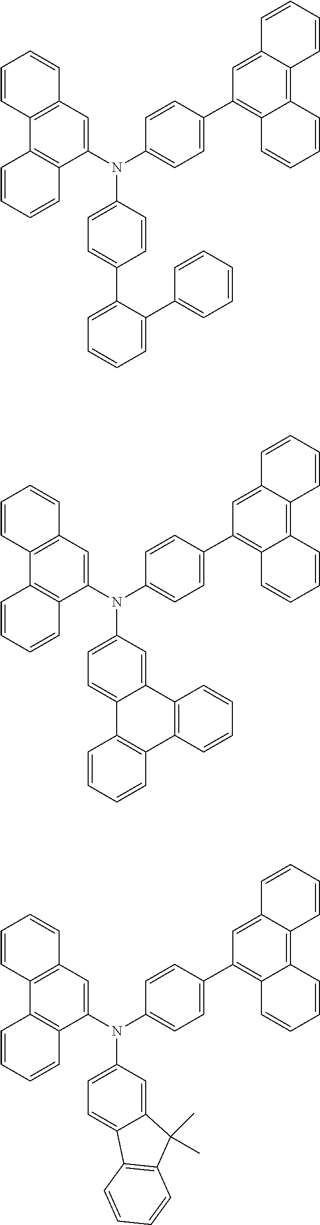 [SMILES: c1ccc(-c2ccccc2-c2ccc(N(c3ccc(-c4cc5ccccc5c5ccccc45)cc3)c3cc4ccccc4c4ccccc34)cc2)cc1 ; c1ccc2c(c1)cc(-c1ccc(N(c3ccc4c5ccccc5c5ccccc5c4c3)c3cc4ccccc4c4ccccc34)cc1)c1ccccc12 ; CC1(C)c2ccccc2-c2ccc(N(c3ccc(-c4cc5ccccc5c5ccccc45)cc3)c3cc4ccccc4c4ccccc34)cc21] 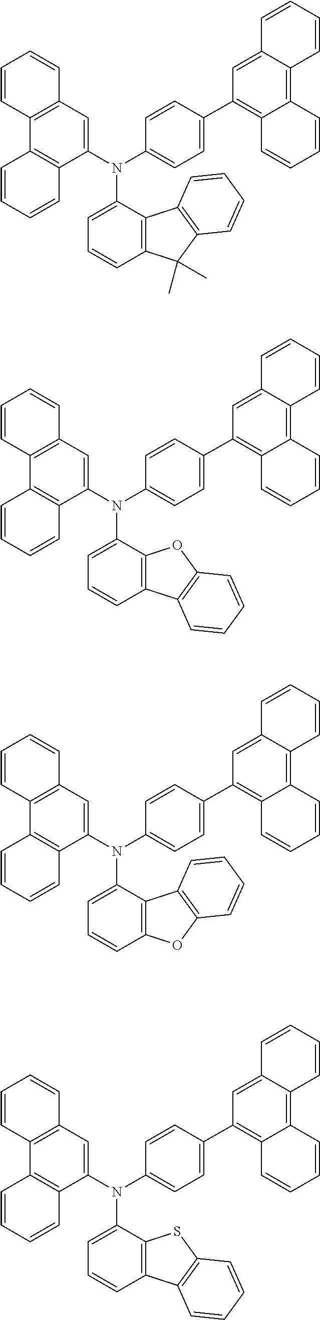 [SMILES: CC1(C)c2ccccc2-c2c(N(c3ccc(-c4cc5ccccc5c5ccccc45)cc3)c3cc4ccccc4c4ccccc34)cccc21 ; c1ccc2c(c1)cc(-c1ccc(N(c3cc4ccccc4c4ccccc34)c3cccc4c3oc3ccccc34)cc1)c1ccccc12 ; c1ccc2c(c1)cc(-c1ccc(N(c3cc4ccccc4c4ccccc34)c3cccc4oc5ccccc5c34)cc1)c1ccccc12 ; c1ccc2c(c1)cc(-c1ccc(N(c3cc4ccccc4c4ccccc34)c3cccc4c3sc3ccccc34)cc1)c1ccccc12] 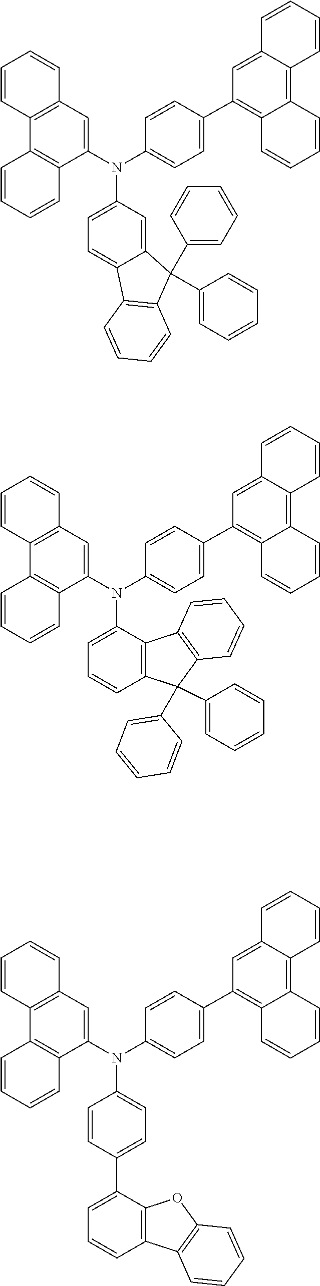 [SMILES: c1ccc(C2(c3ccccc3)c3ccccc3-c3ccc(N(c4ccc(-c5cc6ccccc6c6ccccc56)cc4)c4cc5ccccc5c5ccccc45)cc32)cc1 ; c1ccc(C2(c3ccccc3)c3ccccc3-c3c(N(c4ccc(-c5cc6ccccc6c6ccccc56)cc4)c4cc5ccccc5c5ccccc45)cccc32)cc1 ; c1ccc2c(c1)cc(-c1ccc(N(c3ccc(-c4cccc5c4oc4ccccc45)cc3)c3cc4ccccc4c4ccccc34)cc1)c1ccccc12] 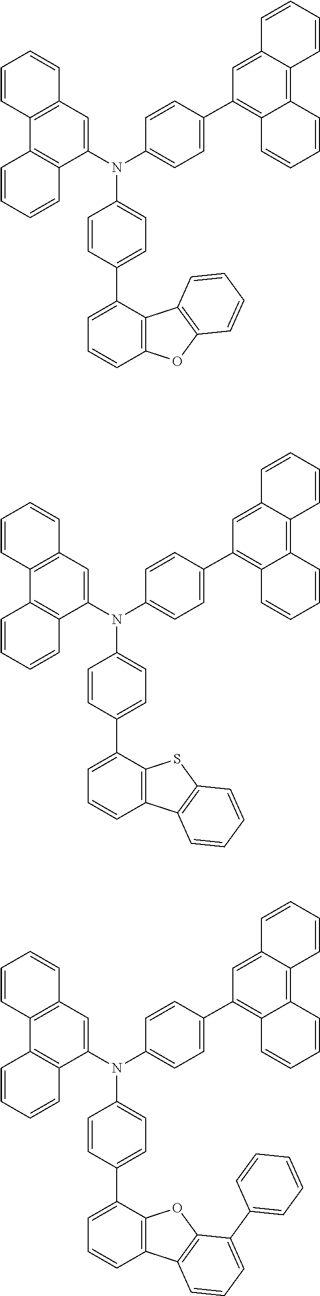 [SMILES: c1ccc2c(c1)cc(-c1ccc(N(c3ccc(-c4cccc5oc6ccccc6c45)cc3)c3cc4ccccc4c4ccccc34)cc1)c1ccccc12 ; c1ccc2c(c1)cc(-c1ccc(N(c3ccc(-c4cccc5c4sc4ccccc45)cc3)c3cc4ccccc4c4ccccc34)cc1)c1ccccc12 ; c1ccc(-c2cccc3c2oc2c(-c4ccc(N(c5ccc(-c6cc7ccccc7c7ccccc67)cc5)c5cc6ccccc6c6ccccc56)cc4)cccc23)cc1] 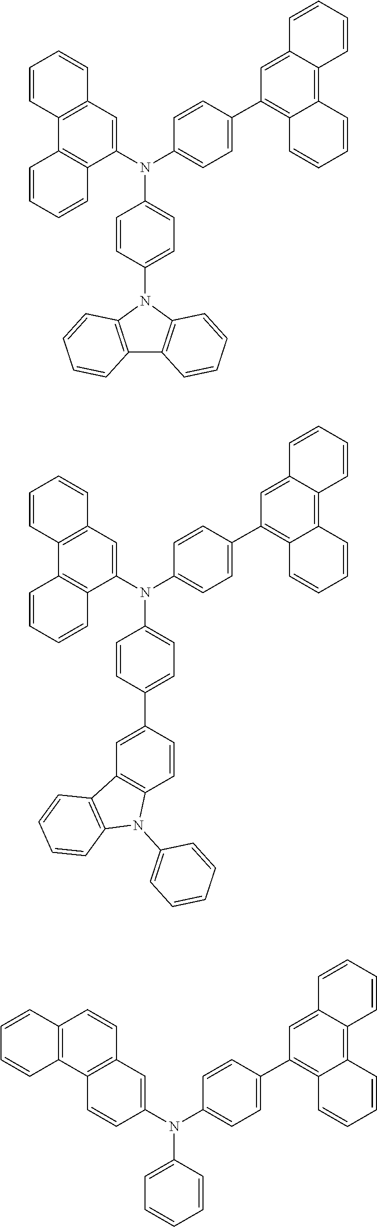 [SMILES: c1ccc2c(c1)cc(-c1ccc(N(c3ccc(-n4c5ccccc5c5ccccc54)cc3)c3cc4ccccc4c4ccccc34)cc1)c1ccccc12 ; c1ccc(-n2c3ccccc3c3cc(-c4ccc(N(c5ccc(-c6cc7ccccc7c7ccccc67)cc5)c5cc6ccccc6c6ccccc56)cc4)ccc32)cc1 ; c1ccc(N(c2ccc(-c3cc4ccccc4c4ccccc34)cc2)c2ccc3c(ccc4ccccc43)c2)cc1] 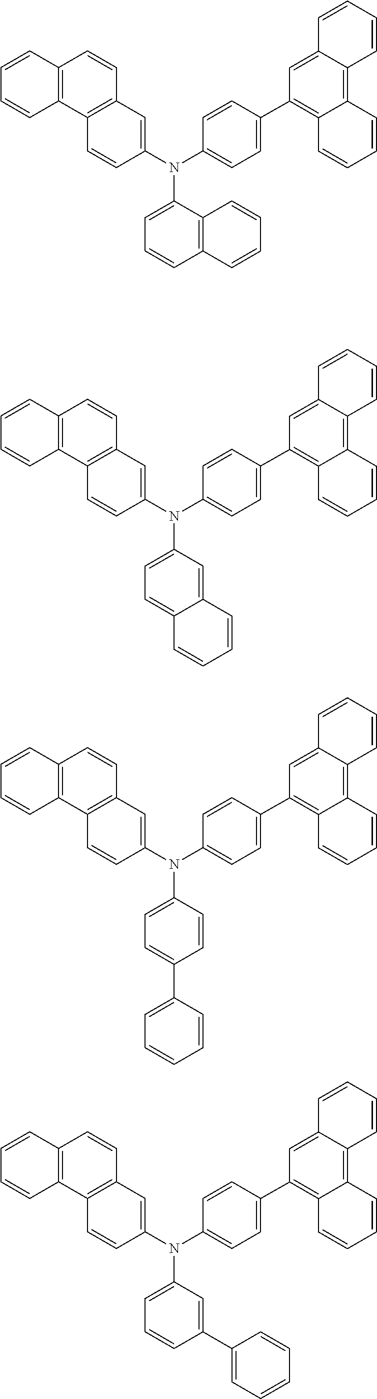 [SMILES: c1ccc2c(N(c3ccc(-c4cc5ccccc5c5ccccc45)cc3)c3ccc4c(ccc5ccccc54)c3)cccc2c1 ; c1ccc2cc(N(c3ccc(-c4cc5ccccc5c5ccccc45)cc3)c3ccc4c(ccc5ccccc54)c3)ccc2c1 ; c1ccc(-c2ccc(N(c3ccc(-c4cc5ccccc5c5ccccc45)cc3)c3ccc4c(ccc5ccccc54)c3)cc2)cc1 ; c1ccc(-c2cccc(N(c3ccc(-c4cc5ccccc5c5ccccc45)cc3)c3ccc4c(ccc5ccccc54)c3)c2)cc1] 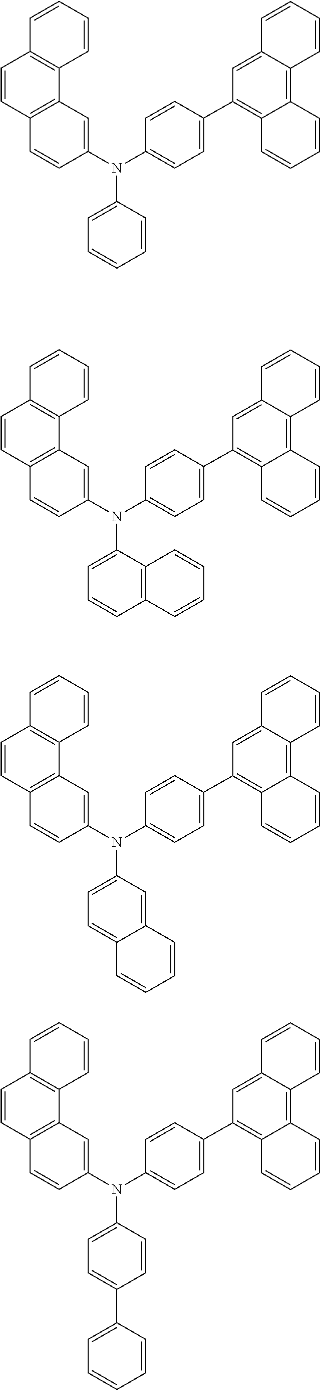 [SMILES: c1ccc(N(c2ccc(-c3cc4ccccc4c4ccccc34)cc2)c2ccc3ccc4ccccc4c3c2)cc1 ; c1ccc2c(N(c3ccc(-c4cc5ccccc5c5ccccc45)cc3)c3ccc4ccc5ccccc5c4c3)cccc2c1 ; c1ccc2cc(N(c3ccc(-c4cc5ccccc5c5ccccc45)cc3)c3ccc4ccc5ccccc5c4c3)ccc2c1 ; c1ccc(-c2ccc(N(c3ccc(-c4cc5ccccc5c5ccccc45)cc3)c3ccc4ccc5ccccc5c4c3)cc2)cc1] 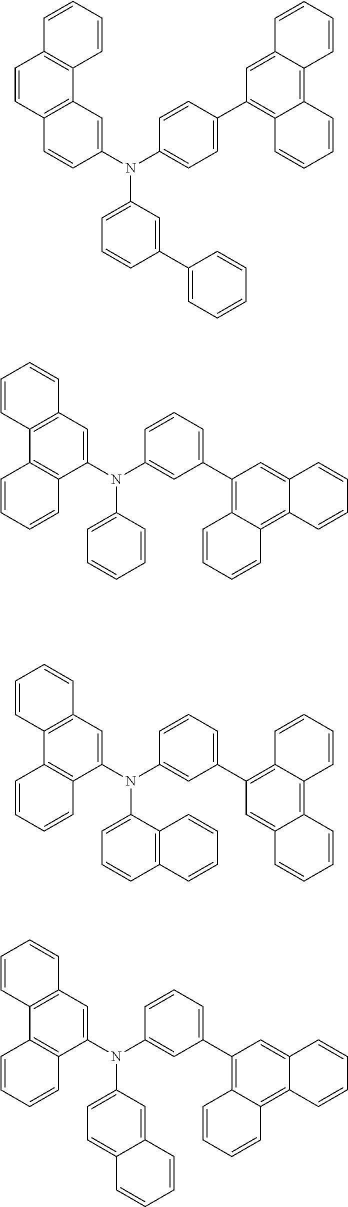 [SMILES: c1ccc(-c2cccc(N(c3ccc(-c4cc5ccccc5c5ccccc45)cc3)c3ccc4ccc5ccccc5c4c3)c2)cc1 ; c1ccc(N(c2cccc(-c3cc4ccccc4c4ccccc34)c2)c2cc3ccccc3c3ccccc23)cc1 ; c1cc(-c2cc3ccccc3c3ccccc23)cc(N(c2cccc3ccccc23)c2cc3ccccc3c3ccccc23)c1 ; c1cc(-c2cc3ccccc3c3ccccc23)cc(N(c2ccc3ccccc3c2)c2cc3ccccc3c3ccccc23)c1] 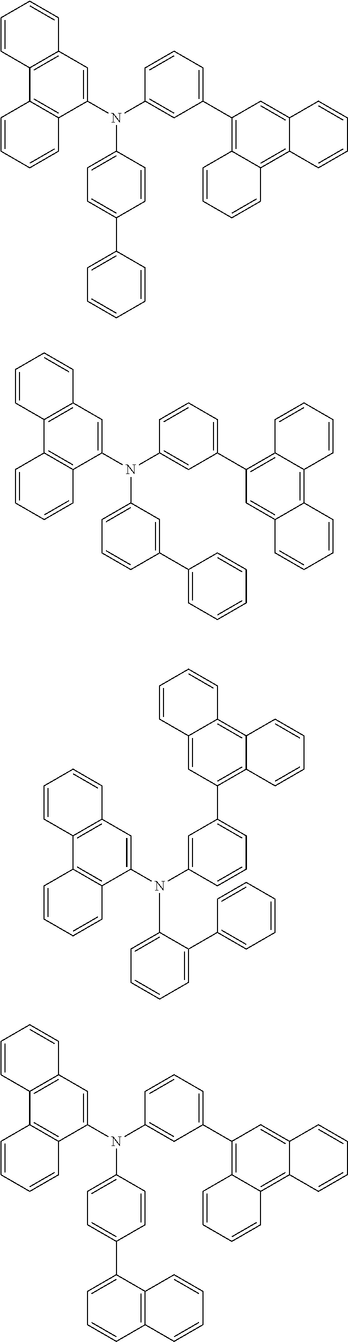 [SMILES: c1ccc(-c2ccc(N(c3cccc(-c4cc5ccccc5c5ccccc45)c3)c3cc4ccccc4c4ccccc34)cc2)cc1 ; c1ccc(-c2cccc(N(c3cccc(-c4cc5ccccc5c5ccccc45)c3)c3cc4ccccc4c4ccccc34)c2)cc1 ; c1ccc(-c2ccccc2N(c2cccc(-c3cc4ccccc4c4ccccc34)c2)c2cc3ccccc3c3ccccc23)cc1 ; c1cc(-c2cc3ccccc3c3ccccc23)cc(N(c2ccc(-c3cccc4ccccc34)cc2)c2cc3ccccc3c3ccccc23)c1] 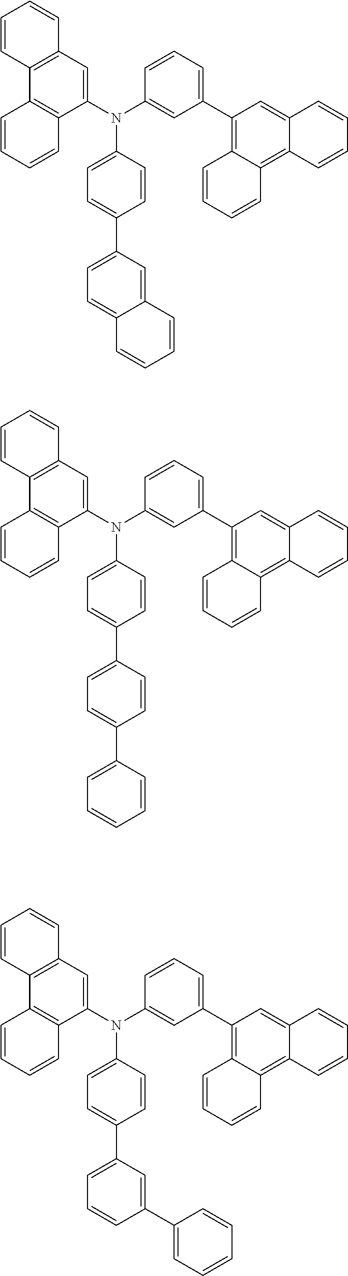 [SMILES: c1cc(-c2cc3ccccc3c3ccccc23)cc(N(c2ccc(-c3ccc4ccccc4c3)cc2)c2cc3ccccc3c3ccccc23)c1 ; c1ccc(-c2ccc(-c3ccc(N(c4cccc(-c5cc6ccccc6c6ccccc56)c4)c4cc5ccccc5c5ccccc45)cc3)cc2)cc1 ; c1ccc(-c2cccc(-c3ccc(N(c4cccc(-c5cc6ccccc6c6ccccc56)c4)c4cc5ccccc5c5ccccc45)cc3)c2)cc1] 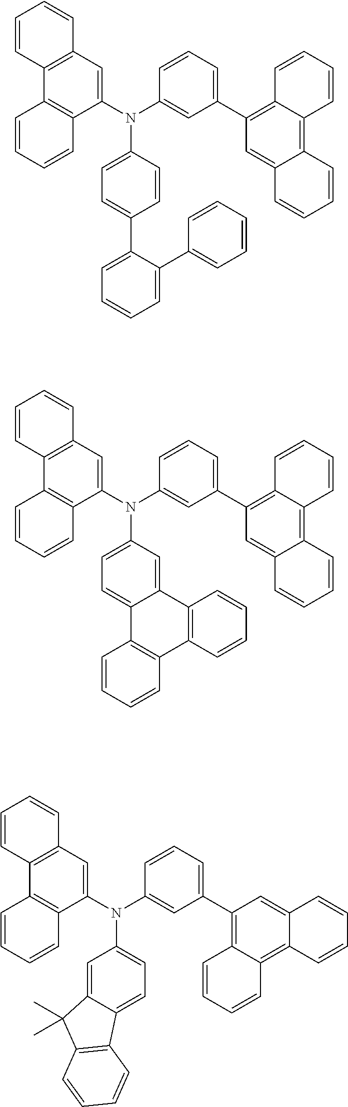 [SMILES: c1ccc(-c2ccccc2-c2ccc(N(c3cccc(-c4cc5ccccc5c5ccccc45)c3)c3cc4ccccc4c4ccccc34)cc2)cc1 ; c1cc(-c2cc3ccccc3c3ccccc23)cc(N(c2ccc3c4ccccc4c4ccccc4c3c2)c2cc3ccccc3c3ccccc23)c1 ; CC1(C)c2ccccc2-c2ccc(N(c3cccc(-c4cc5ccccc5c5ccccc45)c3)c3cc4ccccc4c4ccccc34)cc21] 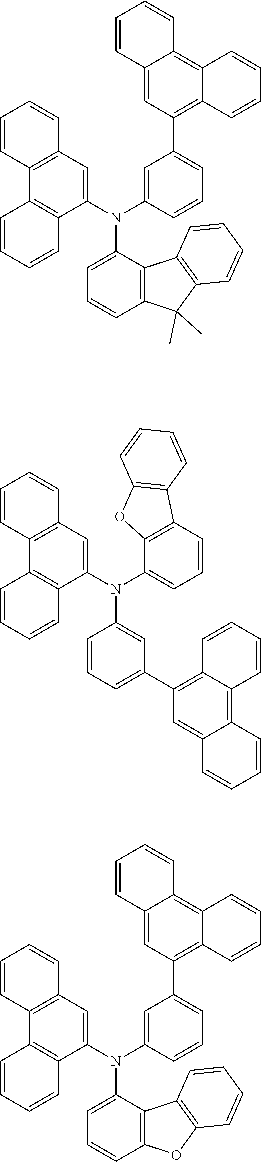 [SMILES: CC1(C)c2ccccc2-c2c(N(c3cccc(-c4cc5ccccc5c5ccccc45)c3)c3cc4ccccc4c4ccccc34)cccc21 ; c1cc(-c2cc3ccccc3c3ccccc23)cc(N(c2cc3ccccc3c3ccccc23)c2cccc3c2oc2ccccc23)c1 ; c1cc(-c2cc3ccccc3c3ccccc23)cc(N(c2cc3ccccc3c3ccccc23)c2cccc3oc4ccccc4c23)c1] 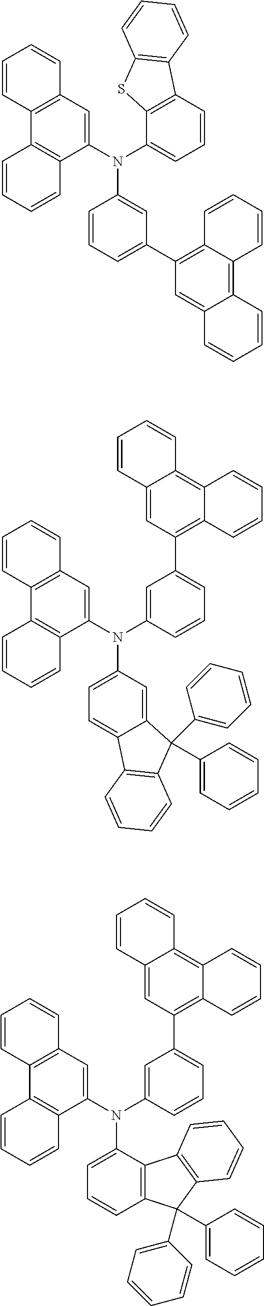 [SMILES: c1cc(-c2cc3ccccc3c3ccccc23)cc(N(c2cc3ccccc3c3ccccc23)c2cccc3c2sc2ccccc23)c1 ; c1ccc(C2(c3ccccc3)c3ccccc3-c3ccc(N(c4cccc(-c5cc6ccccc6c6ccccc56)c4)c4cc5ccccc5c5ccccc45)cc32)cc1 ; c1ccc(C2(c3ccccc3)c3ccccc3-c3c(N(c4cccc(-c5cc6ccccc6c6ccccc56)c4)c4cc5ccccc5c5ccccc45)cccc32)cc1] 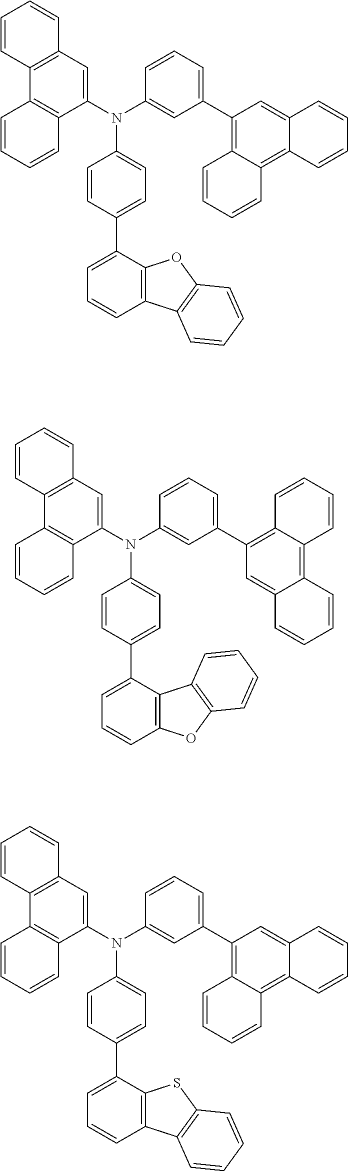 [SMILES: c1cc(-c2cc3ccccc3c3ccccc23)cc(N(c2ccc(-c3cccc4c3oc3ccccc34)cc2)c2cc3ccccc3c3ccccc23)c1 ; c1cc(-c2cc3ccccc3c3ccccc23)cc(N(c2ccc(-c3cccc4oc5ccccc5c34)cc2)c2cc3ccccc3c3ccccc23)c1 ; c1cc(-c2cc3ccccc3c3ccccc23)cc(N(c2ccc(-c3cccc4c3sc3ccccc34)cc2)c2cc3ccccc3c3ccccc23)c1] 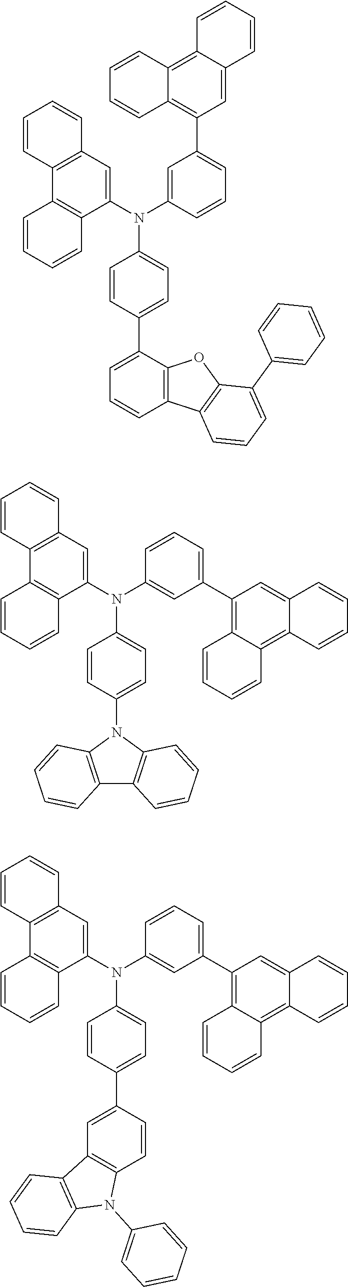 [SMILES: c1ccc(-c2cccc3c2oc2c(-c4ccc(N(c5cccc(-c6cc7ccccc7c7ccccc67)c5)c5cc6ccccc6c6ccccc56)cc4)cccc23)cc1 ; c1cc(-c2cc3ccccc3c3ccccc23)cc(N(c2ccc(-n3c4ccccc4c4ccccc43)cc2)c2cc3ccccc3c3ccccc23)c1 ; c1ccc(-n2c3ccccc3c3cc(-c4ccc(N(c5cccc(-c6cc7ccccc7c7ccccc67)c5)c5cc6ccccc6c6ccccc56)cc4)ccc32)cc1] 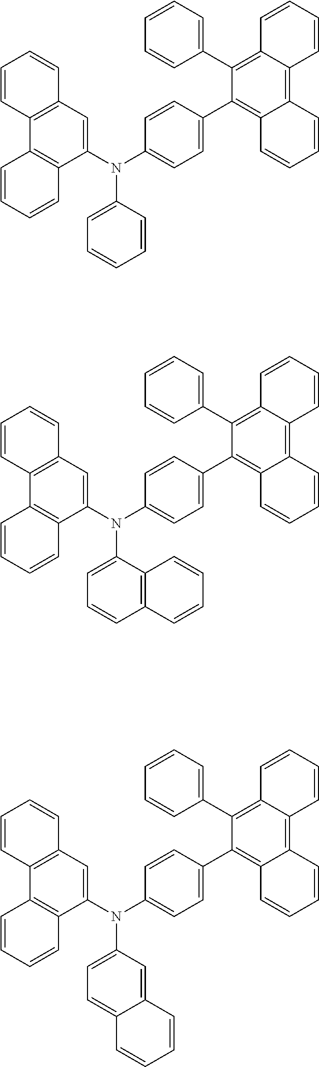 [SMILES: c1ccc(-c2c(-c3ccc(N(c4ccccc4)c4cc5ccccc5c5ccccc45)cc3)c3ccccc3c3ccccc23)cc1 ; c1ccc(-c2c(-c3ccc(N(c4cccc5ccccc45)c4cc5ccccc5c5ccccc45)cc3)c3ccccc3c3ccccc23)cc1 ; c1ccc(-c2c(-c3ccc(N(c4ccc5ccccc5c4)c4cc5ccccc5c5ccccc45)cc3)c3ccccc3c3ccccc23)cc1] 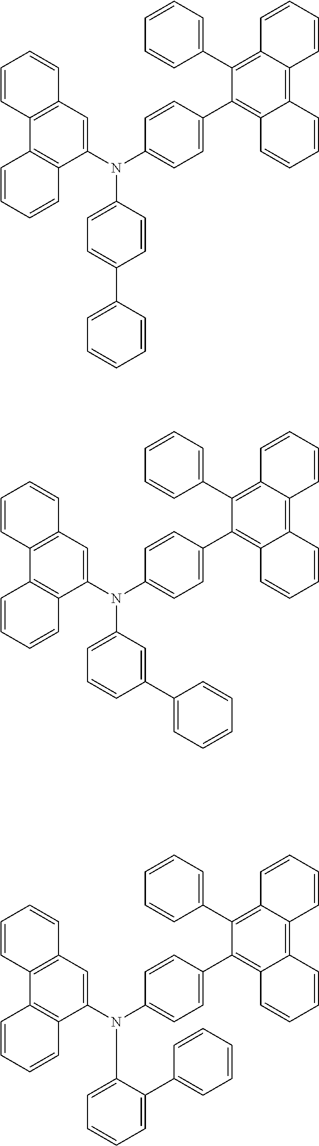 [SMILES: c1ccc(-c2ccc(N(c3ccc(-c4c(-c5ccccc5)c5ccccc5c5ccccc45)cc3)c3cc4ccccc4c4ccccc34)cc2)cc1 ; c1ccc(-c2cccc(N(c3ccc(-c4c(-c5ccccc5)c5ccccc5c5ccccc45)cc3)c3cc4ccccc4c4ccccc34)c2)cc1 ; c1ccc(-c2ccccc2N(c2ccc(-c3c(-c4ccccc4)c4ccccc4c4ccccc34)cc2)c2cc3ccccc3c3ccccc23)cc1] 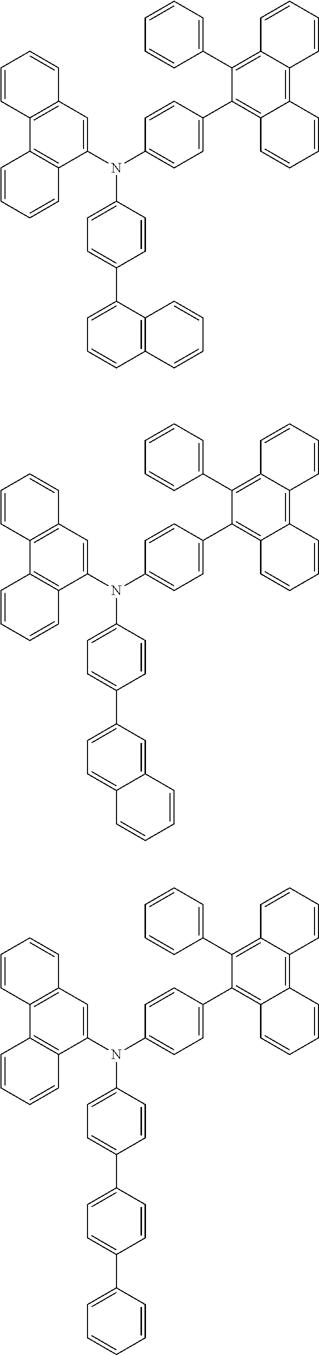 [SMILES: c1ccc(-c2c(-c3ccc(N(c4ccc(-c5cccc6ccccc56)cc4)c4cc5ccccc5c5ccccc45)cc3)c3ccccc3c3ccccc23)cc1 ; c1ccc(-c2c(-c3ccc(N(c4ccc(-c5ccc6ccccc6c5)cc4)c4cc5ccccc5c5ccccc45)cc3)c3ccccc3c3ccccc23)cc1 ; c1ccc(-c2ccc(-c3ccc(N(c4ccc(-c5c(-c6ccccc6)c6ccccc6c6ccccc56)cc4)c4cc5ccccc5c5ccccc45)cc3)cc2)cc1] 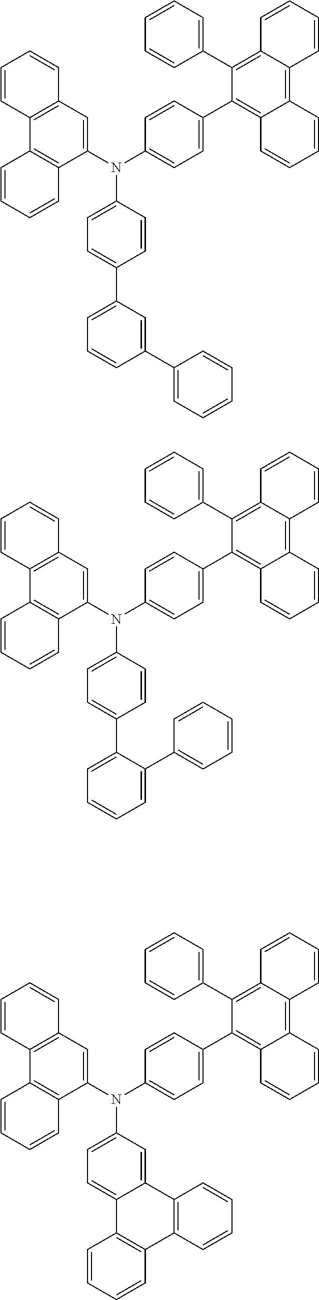 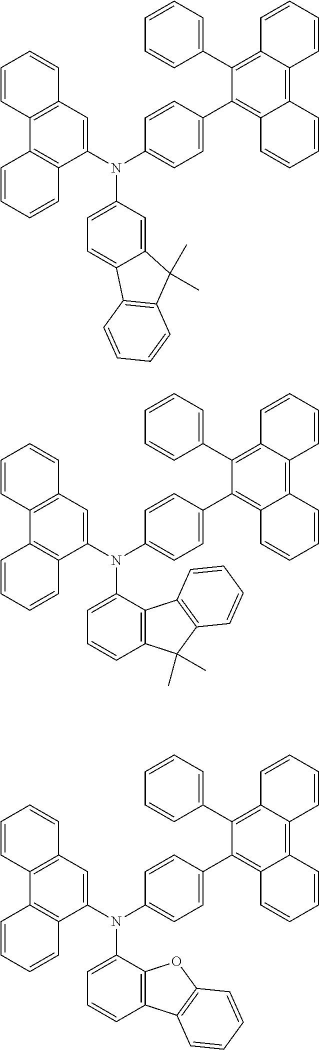 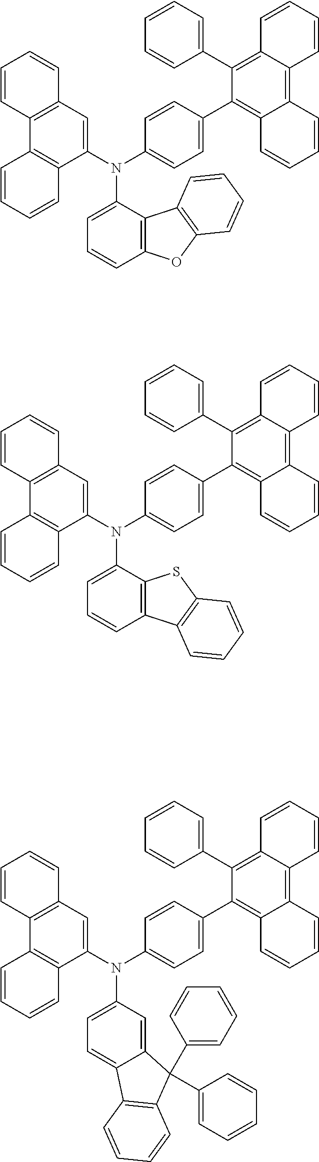 [SMILES: c1ccc(-c2c(-c3ccc(N(c4cc5ccccc5c5ccccc45)c4cccc5oc6ccccc6c45)cc3)c3ccccc3c3ccccc23)cc1 ; c1ccc(-c2c(-c3ccc(N(c4cc5ccccc5c5ccccc45)c4cccc5c4sc4ccccc45)cc3)c3ccccc3c3ccccc23)cc1 ; c1ccc(-c2c(-c3ccc(N(c4ccc5c(c4)C(c4ccccc4)(c4ccccc4)c4ccccc4-5)c4cc5ccccc5c5ccccc45)cc3)c3ccccc3c3ccccc23)cc1] 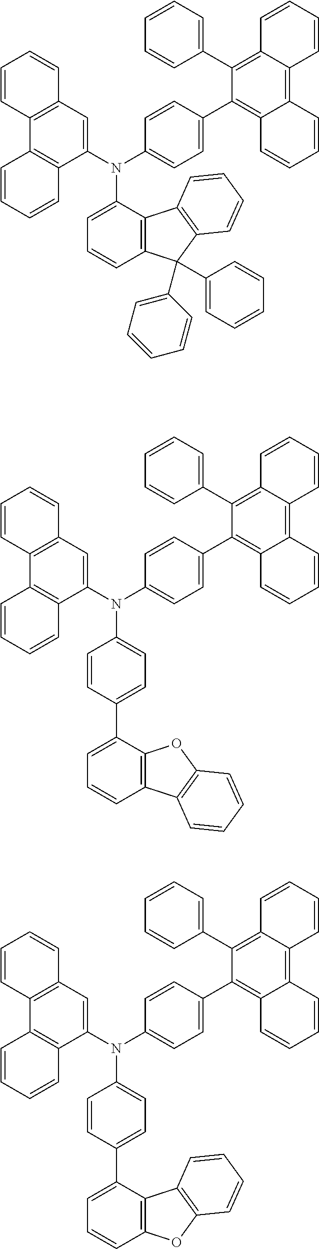 [SMILES: c1ccc(-c2c(-c3ccc(N(c4cccc5c4-c4ccccc4C5(c4ccccc4)c4ccccc4)c4cc5ccccc5c5ccccc45)cc3)c3ccccc3c3ccccc23)cc1 ; c1ccc(-c2c(-c3ccc(N(c4ccc(-c5cccc6c5oc5ccccc56)cc4)c4cc5ccccc5c5ccccc45)cc3)c3ccccc3c3ccccc23)cc1 ; c1ccc(-c2c(-c3ccc(N(c4ccc(-c5cccc6oc7ccccc7c56)cc4)c4cc5ccccc5c5ccccc45)cc3)c3ccccc3c3ccccc23)cc1] 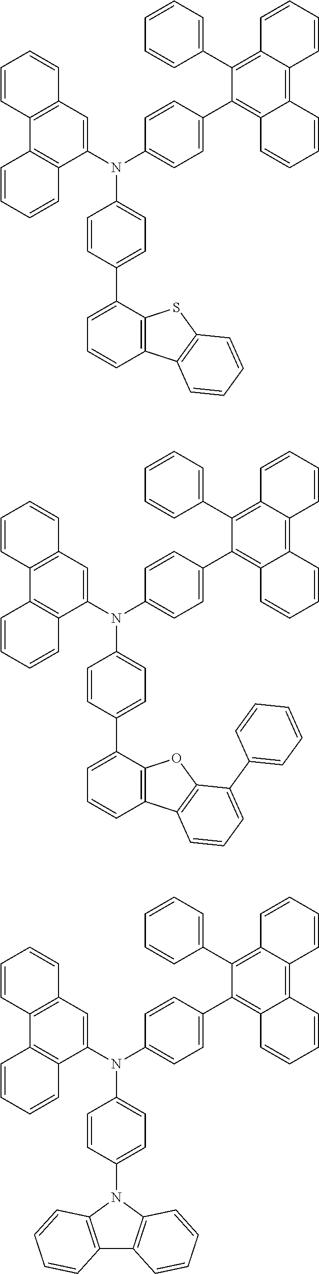 [SMILES: c1ccc(-c2c(-c3ccc(N(c4ccc(-c5cccc6c5sc5ccccc56)cc4)c4cc5ccccc5c5ccccc45)cc3)c3ccccc3c3ccccc23)cc1 ; c1ccc(-c2c(-c3ccc(N(c4ccc(-c5cccc6c5oc5c(-c7ccccc7)cccc56)cc4)c4cc5ccccc5c5ccccc45)cc3)c3ccccc3c3ccccc23)cc1 ; c1ccc(-c2c(-c3ccc(N(c4ccc(-n5c6ccccc6c6ccccc65)cc4)c4cc5ccccc5c5ccccc45)cc3)c3ccccc3c3ccccc23)cc1] 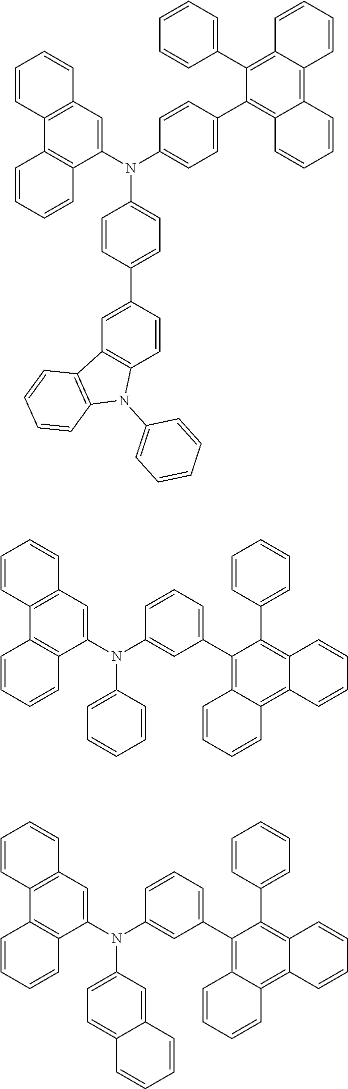 [SMILES: c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1 ; c1ccc(-c2c(-c3cccc(N(c4ccccc4)c4cc5ccccc5c5ccccc45)c3)c3ccccc3c3ccccc23)cc1 ; c1ccc(-c2c(-c3cccc(N(c4ccc5ccccc5c4)c4cc5ccccc5c5ccccc45)c3)c3ccccc3c3ccccc23)cc1] 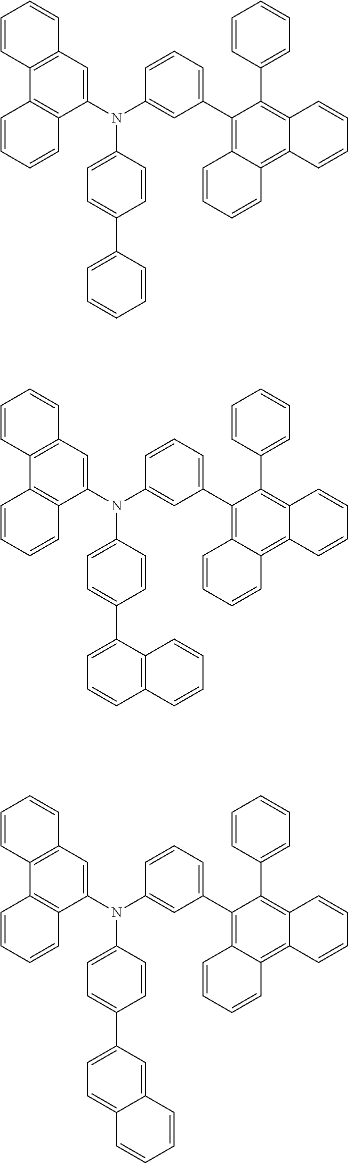 [SMILES: c1ccc(-c2ccc(N(c3cccc(-c4c(-c5ccccc5)c5ccccc5c5ccccc45)c3)c3cc4ccccc4c4ccccc34)cc2)cc1 ; c1ccc(-c2c(-c3cccc(N(c4ccc(-c5cccc6ccccc56)cc4)c4cc5ccccc5c5ccccc45)c3)c3ccccc3c3ccccc23)cc1 ; c1ccc(-c2c(-c3cccc(N(c4ccc(-c5ccc6ccccc6c5)cc4)c4cc5ccccc5c5ccccc45)c3)c3ccccc3c3ccccc23)cc1] 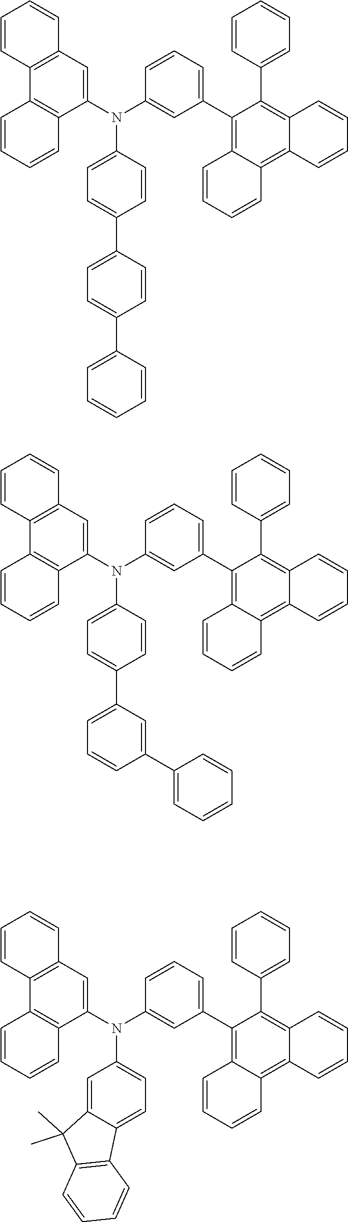 [SMILES: c1ccc(-c2ccc(-c3ccc(N(c4cccc(-c5c(-c6ccccc6)c6ccccc6c6ccccc56)c4)c4cc5ccccc5c5ccccc45)cc3)cc2)cc1 ; c1ccc(-c2cccc(-c3ccc(N(c4cccc(-c5c(-c6ccccc6)c6ccccc6c6ccccc56)c4)c4cc5ccccc5c5ccccc45)cc3)c2)cc1 ; CC1(C)c2ccccc2-c2ccc(N(c3cccc(-c4c(-c5ccccc5)c5ccccc5c5ccccc45)c3)c3cc4ccccc4c4ccccc34)cc21] 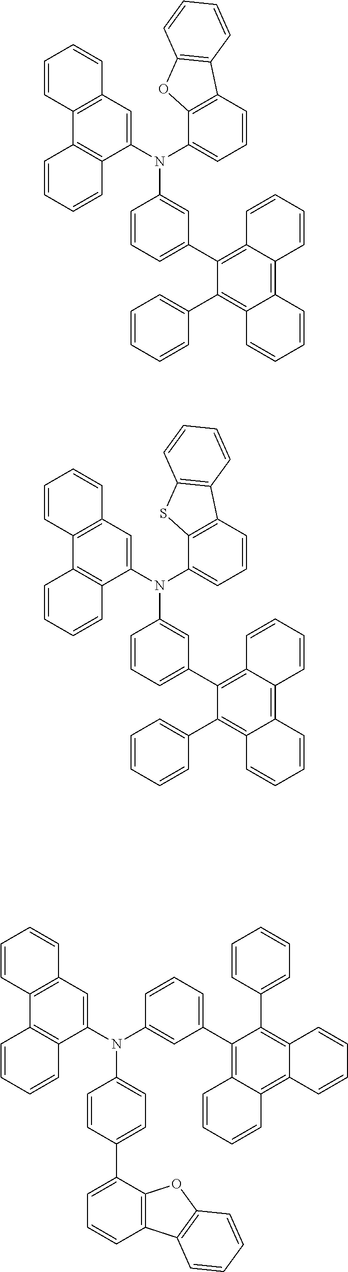 [SMILES: c1ccc(-c2c(-c3cccc(N(c4cc5ccccc5c5ccccc45)c4cccc5c4oc4ccccc45)c3)c3ccccc3c3ccccc23)cc1 ; c1ccc(-c2c(-c3cccc(N(c4cc5ccccc5c5ccccc45)c4cccc5c4sc4ccccc45)c3)c3ccccc3c3ccccc23)cc1 ; c1ccc(-c2c(-c3cccc(N(c4ccc(-c5cccc6c5oc5ccccc56)cc4)c4cc5ccccc5c5ccccc45)c3)c3ccccc3c3ccccc23)cc1] 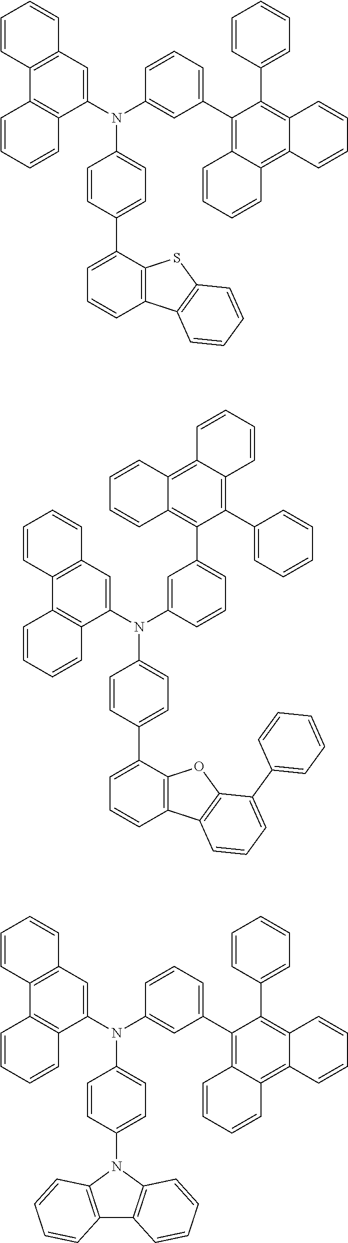 [SMILES: c1ccc(-c2c(-c3cccc(N(c4ccc(-c5cccc6c5sc5ccccc56)cc4)c4cc5ccccc5c5ccccc45)c3)c3ccccc3c3ccccc23)cc1 ; c1ccc(-c2c(-c3cccc(N(c4ccc(-c5cccc6c5oc5c(-c7ccccc7)cccc56)cc4)c4cc5ccccc5c5ccccc45)c3)c3ccccc3c3ccccc23)cc1 ; c1ccc(-c2c(-c3cccc(N(c4ccc(-n5c6ccccc6c6ccccc65)cc4)c4cc5ccccc5c5ccccc45)c3)c3ccccc3c3ccccc23)cc1] 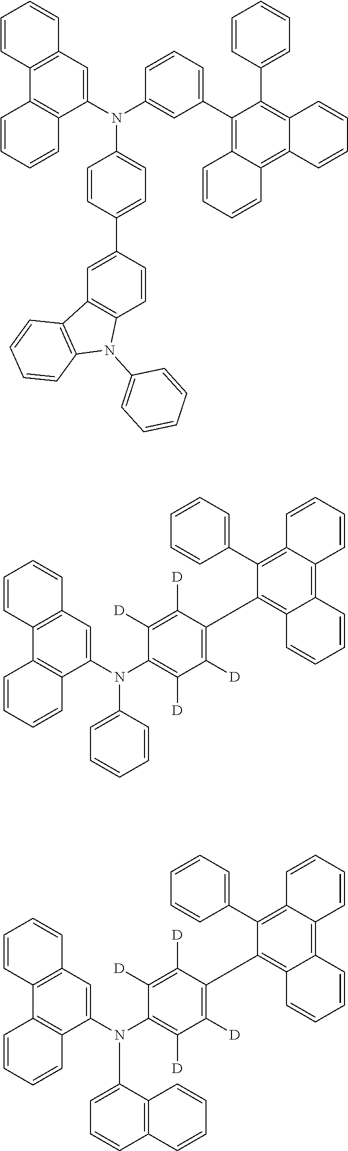 [SMILES: c1ccc(-c2c(-c3cccc(N(c4ccc(-c5ccc6c(c5)c5ccccc5n6-c5ccccc5)cc4)c4cc5ccccc5c5ccccc45)c3)c3ccccc3c3ccccc23)cc1 ; [2H]c1c([2H])c(N(c2ccccc2)c2cc3ccccc3c3ccccc23)c([2H])c([2H])c1-c1c(-c2ccccc2)c2ccccc2c2ccccc12 ; [2H]c1c([2H])c(N(c2cccc3ccccc23)c2cc3ccccc3c3ccccc23)c([2H])c([2H])c1-c1c(-c2ccccc2)c2ccccc2c2ccccc12] 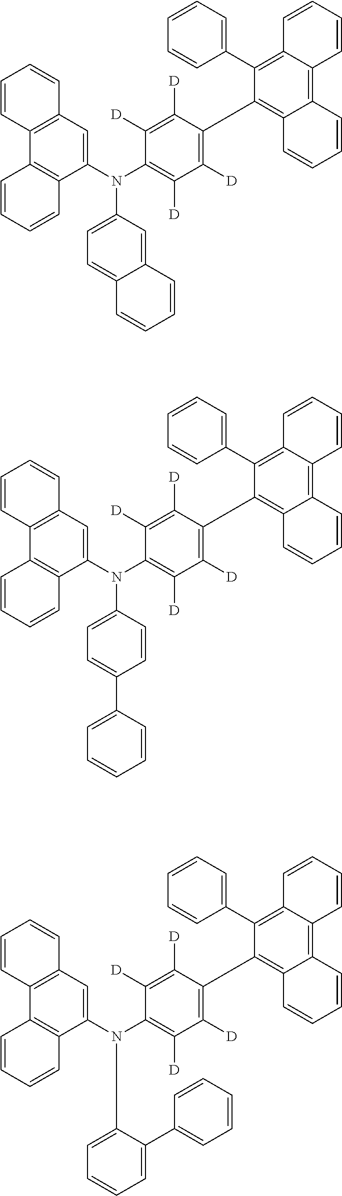 [SMILES: [2H]c1c([2H])c(N(c2ccc3ccccc3c2)c2cc3ccccc3c3ccccc23)c([2H])c([2H])c1-c1c(-c2ccccc2)c2ccccc2c2ccccc12 ; [2H]c1c([2H])c(N(c2ccc(-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccccc2-c2ccccc2)c2cc3ccccc3c3ccccc23)c([2H])c([2H])c1-c1c(-c2ccccc2)c2ccccc2c2ccccc12] 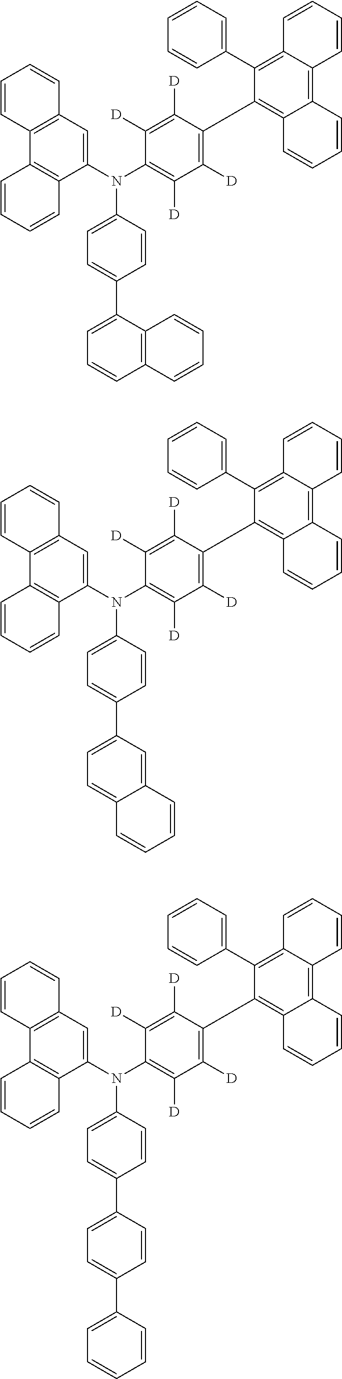 [SMILES: [2H]c1c([2H])c(N(c2ccc(-c3cccc4ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc4ccccc4c3)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3ccc(-c4ccccc4)cc3)cc2)c2cc3ccccc3c3ccccc23)c([2H])c([2H])c1-c1c(-c2ccccc2)c2ccccc2c2ccccc12] 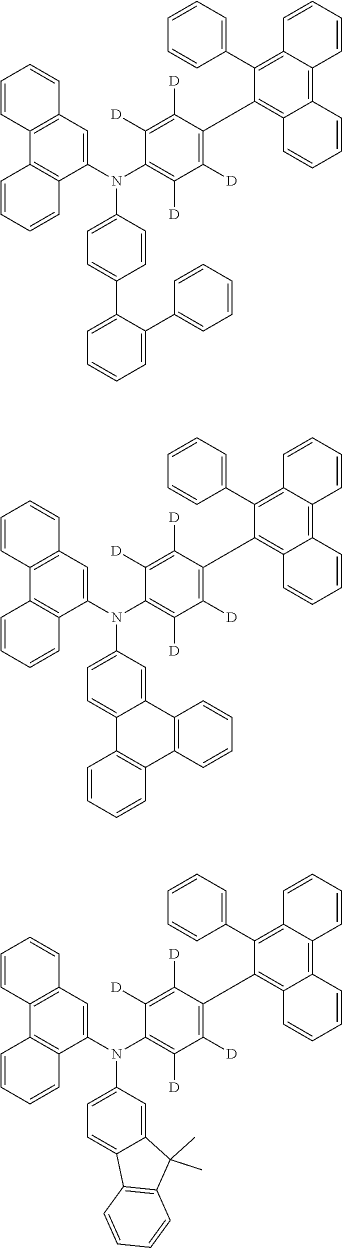 [SMILES: [2H]c1c([2H])c(N(c2ccc(-c3ccccc3-c3ccccc3)cc2)c2cc3ccccc3c3ccccc23)c([2H])c([2H])c1-c1c(-c2ccccc2)c2ccccc2c2ccccc12 ; [2H]c1c([2H])c(N(c2ccc3c4ccccc4c4ccccc4c3c2)c2cc3ccccc3c3ccccc23)c([2H])c([2H])c1-c1c(-c2ccccc2)c2ccccc2c2ccccc12 ; [2H]c1c([2H])c(N(c2ccc3c(c2)C(C)(C)c2ccccc2-3)c2cc3ccccc3c3ccccc23)c([2H])c([2H])c1-c1c(-c2ccccc2)c2ccccc2c2ccccc12] 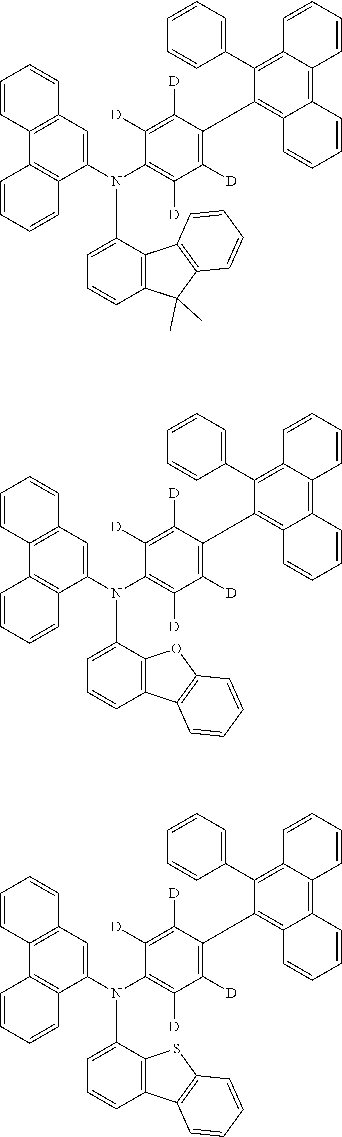 [SMILES: [2H]c1c([2H])c(N(c2cccc3c2-c2ccccc2C3(C)C)c2cc3ccccc3c3ccccc23)c([2H])c([2H])c1-c1c(-c2ccccc2)c2ccccc2c2ccccc12 ; [2H]c1c([2H])c(N(c2cc3ccccc3c3ccccc23)c2cccc3c2oc2ccccc23)c([2H])c([2H])c1-c1c(-c2ccccc2)c2ccccc2c2ccccc12 ; [2H]c1c([2H])c(N(c2cc3ccccc3c3ccccc23)c2cccc3c2sc2ccccc23)c([2H])c([2H])c1-c1c(-c2ccccc2)c2ccccc2c2ccccc12] 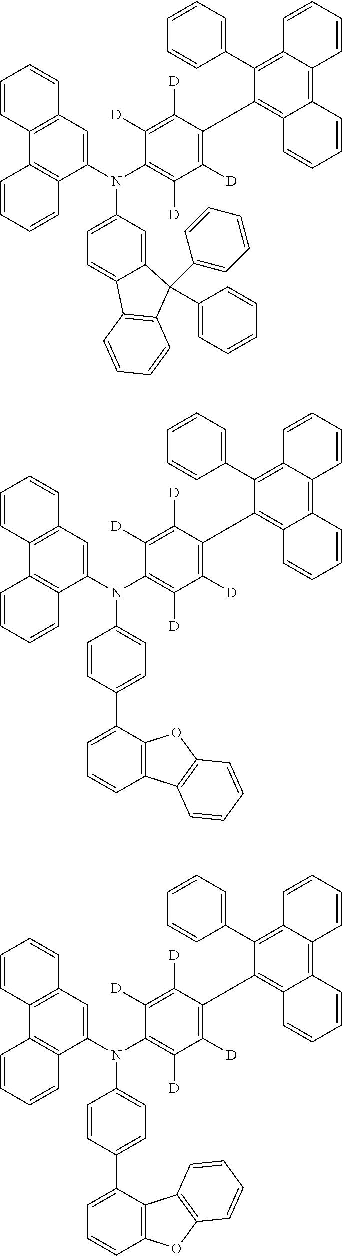 [SMILES: [2H]c1c([2H])c(N(c2ccc3c(c2)C(c2ccccc2)(c2ccccc2)c2ccccc2-3)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4c3oc3ccccc34)cc2)c2cc3ccccc3c3ccccc23)c([2H])c([2H])c1-c1c(-c2ccccc2)c2ccccc2c2ccccc12 ; [2H]c1c([2H])c(N(c2ccc(-c3cccc4oc5ccccc5c34)cc2)c2cc3ccccc3c3ccccc23)c([2H])c([2H])c1-c1c(-c2ccccc2)c2ccccc2c2ccccc12] 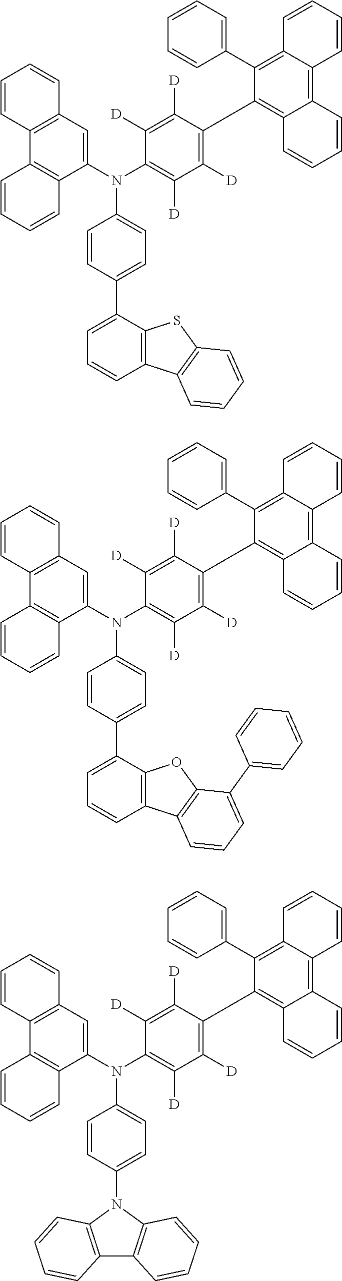 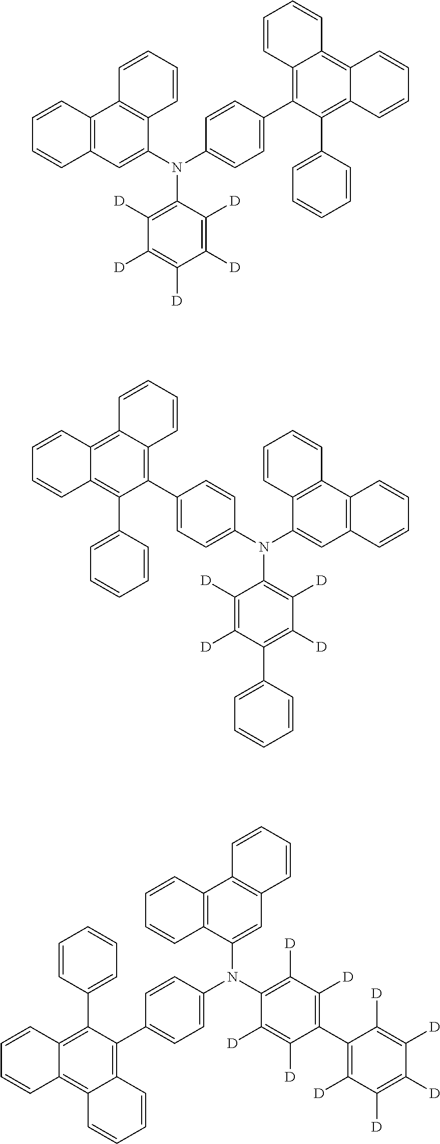 [SMILES: [2H]c1c([2H])c([2H])c(N(c2ccc(-c3c(-c4ccccc4)c4ccccc4c4ccccc34)cc2)c2cc3ccccc3c3ccccc23)c([2H])c1[2H] ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1 ; [2H]c1c([2H])c([2H])c(-c2c([2H])c([2H])c(N(c3ccc(-c4c(-c5ccccc5)c5ccccc5c5ccccc45)cc3)c3cc4ccccc4c4ccccc34)c([2H])c2[2H])c([2H])c1[2H]] 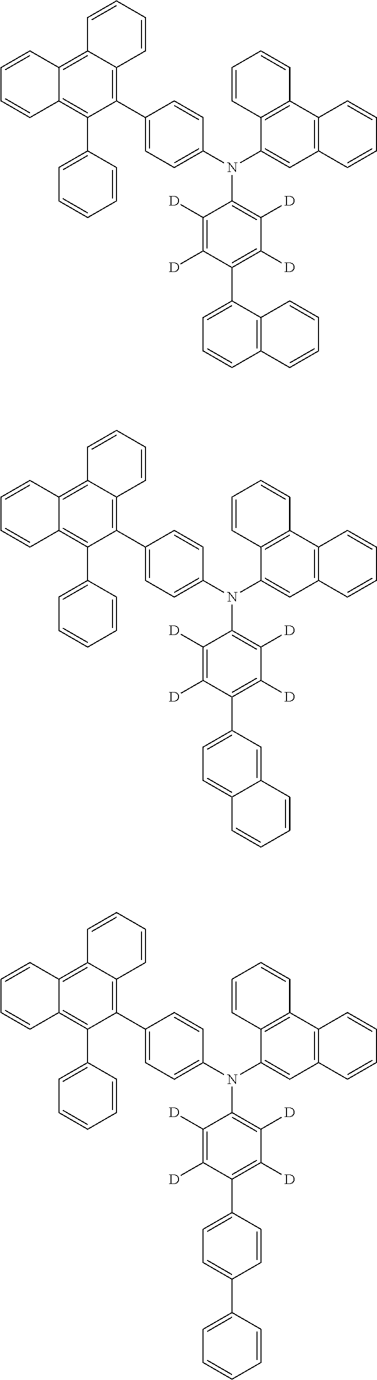 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc2ccccc2c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccc(-c2ccccc2)cc1] 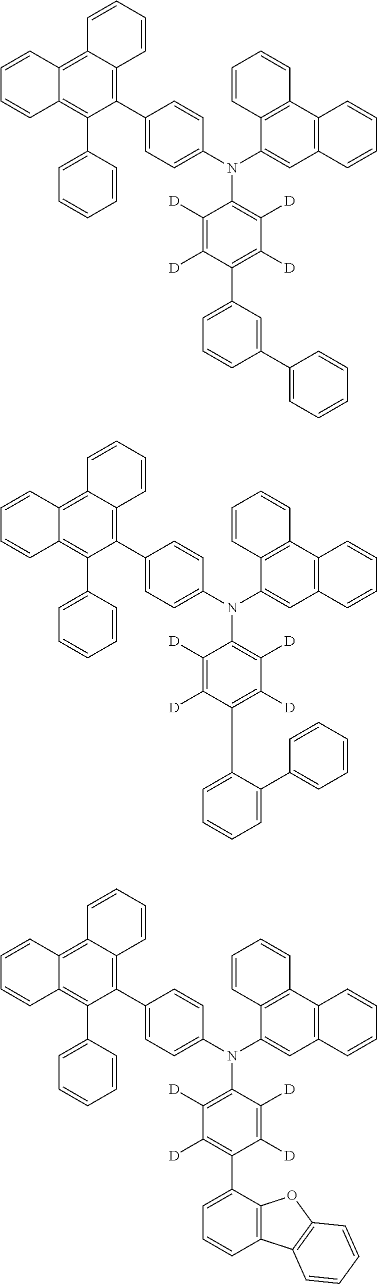 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc(-c2ccccc2)c1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1ccccc1-c1ccccc1 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1oc1ccccc12] 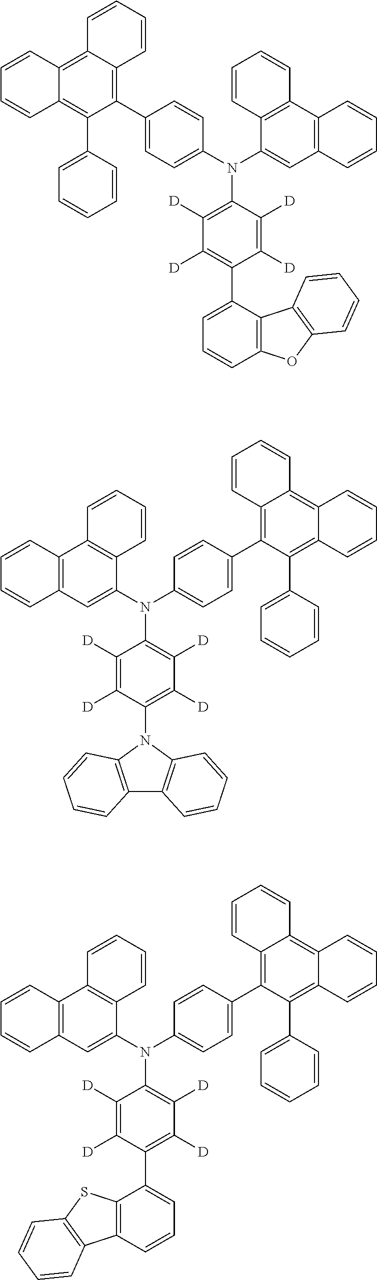 [SMILES: [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2oc3ccccc3c12 ; [2H]c1c([2H])c(-n2c3ccccc3c3ccccc32)c([2H])c([2H])c1N(c1ccc(-c2c(-c3ccccc3)c3ccccc3c3ccccc23)cc1)c1cc2ccccc2c2ccccc12 ; [2H]c1c([2H])c(N(c2ccc(-c3c(-c4ccccc4)c4ccccc4c4ccccc34)cc2)c2cc3ccccc3c3ccccc23)c([2H])c([2H])c1-c1cccc2c1sc1ccccc12] 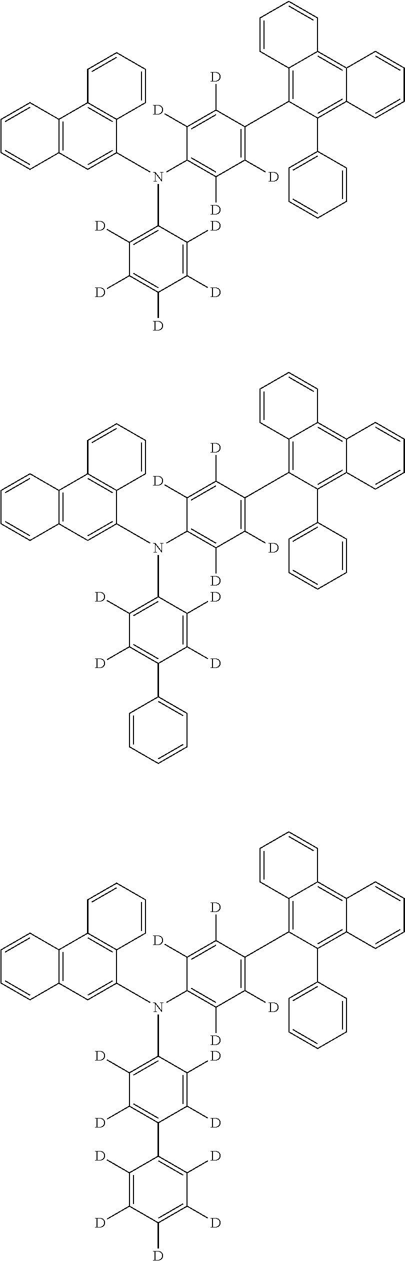 [SMILES: [2H]c1c([2H])c([2H])c(N(c2c([2H])c([2H])c(-c3c(-c4ccccc4)c4ccccc4c4ccccc34)c([2H])c2[2H])c2cc3ccccc3c3ccccc23)c([2H])c1[2H] ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1 ; [2H]c1c([2H])c([2H])c(-c2c([2H])c([2H])c(N(c3c([2H])c([2H])c(-c4c(-c5ccccc5)c5ccccc5c5ccccc45)c([2H])c3[2H])c3cc4ccccc4c4ccccc34)c([2H])c2[2H])c([2H])c1[2H]] 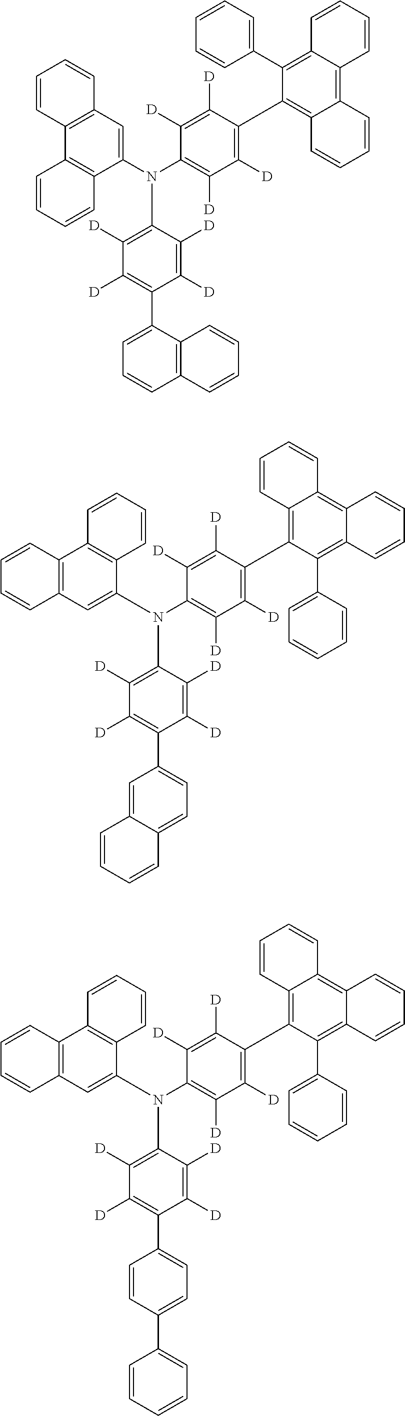 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1cccc2ccccc12 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc(-c2ccccc2)cc1] 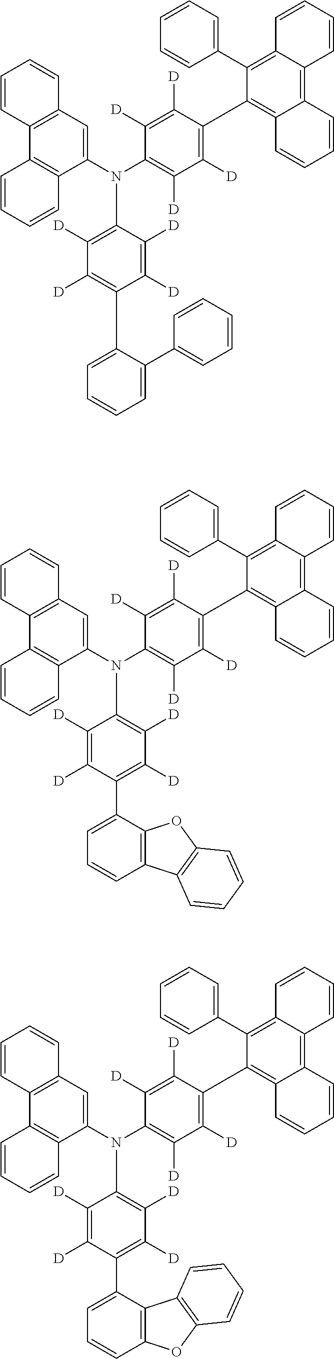 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccccc1-c1ccccc1 ; [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1c(-c2ccccc2)c2ccccc2c2ccccc12] 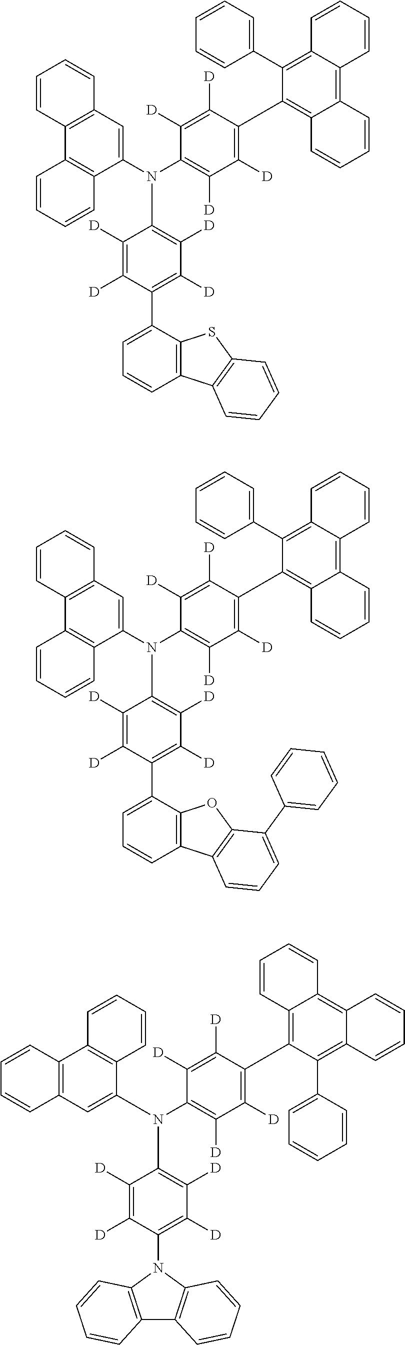 [SMILES: [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1c(-c2ccccc2)c2ccccc2c2ccccc12 ; [2H]c1c([2H])c(N(c2c([2H])c([2H])c(-n3c4ccccc4c4ccccc43)c([2H])c2[2H])c2cc3ccccc3c3ccccc23)c([2H])c([2H])c1-c1c(-c2ccccc2)c2ccccc2c2ccccc12] 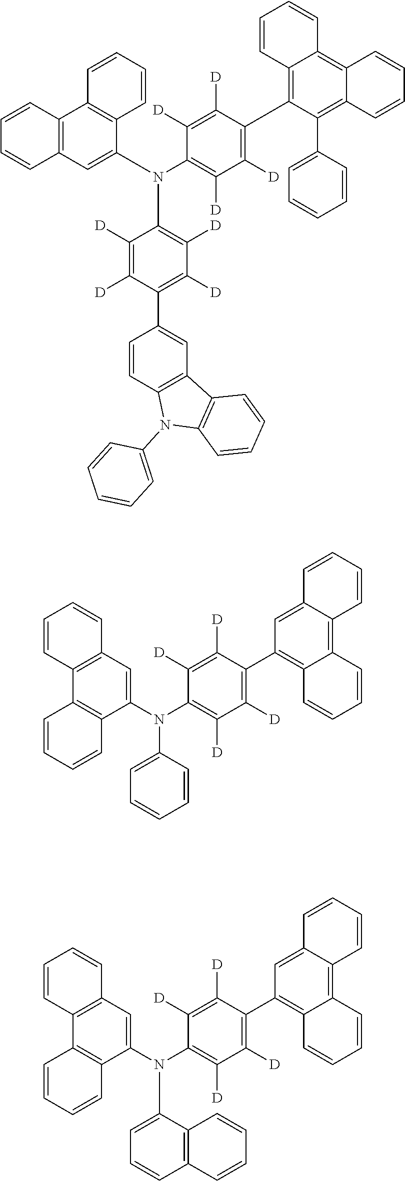 [SMILES: [2H]c1c([2H])c(N(c2c([2H])c([2H])c(-c3c(-c4ccccc4)c4ccccc4c4ccccc34)c([2H])c2[2H])c2cc3ccccc3c3ccccc23)c([2H])c([2H])c1-c1ccc2c(c1)c1ccccc1n2-c1ccccc1 ; [2H]c1c([2H])c(N(c2ccccc2)c2cc3ccccc3c3ccccc23)c([2H])c([2H])c1-c1cc2ccccc2c2ccccc12 ; [2H]c1c([2H])c(N(c2cccc3ccccc23)c2cc3ccccc3c3ccccc23)c([2H])c([2H])c1-c1cc2ccccc2c2ccccc12] 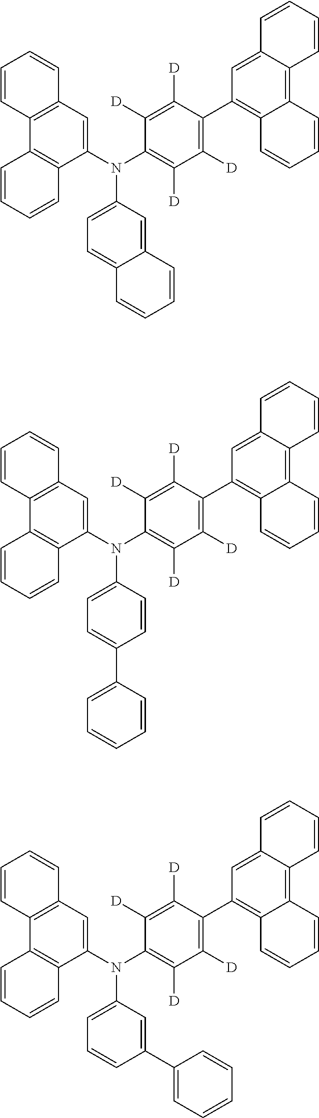 [SMILES: [2H]c1c([2H])c(N(c2ccc3ccccc3c2)c2cc3ccccc3c3ccccc23)c([2H])c([2H])c1-c1cc2ccccc2c2ccccc12 ; [2H]c1c([2H])c(N(c2ccc(-c3ccccc3)cc2)c2cc3ccccc3c3ccccc23)c([2H])c([2H])c1-c1cc2ccccc2c2ccccc12 ; [2H]c1c([2H])c(N(c2cccc(-c3ccccc3)c2)c2cc3ccccc3c3ccccc23)c([2H])c([2H])c1-c1cc2ccccc2c2ccccc12] 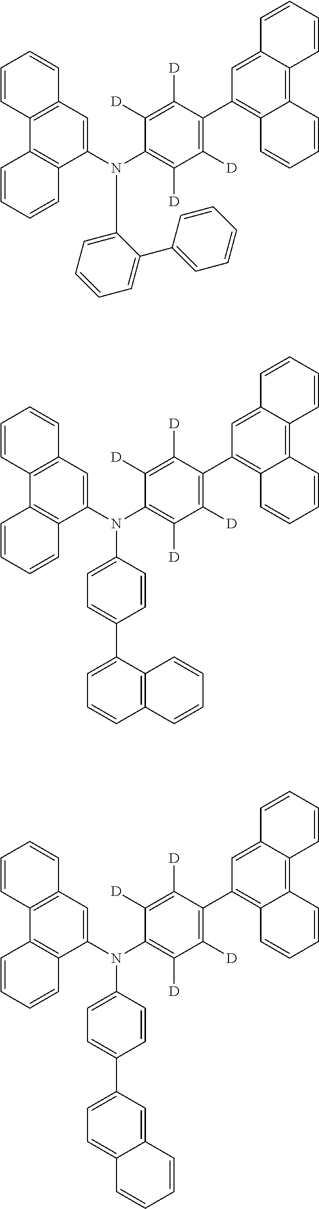 [SMILES: [2H]c1c([2H])c(N(c2ccccc2-c2ccccc2)c2cc3ccccc3c3ccccc23)c([2H])c([2H])c1-c1cc2ccccc2c2ccccc12 ; [2H]c1c([2H])c(N(c2ccc(-c3cccc4ccccc34)cc2)c2cc3ccccc3c3ccccc23)c([2H])c([2H])c1-c1cc2ccccc2c2ccccc12 ; [2H]c1c([2H])c(N(c2ccc(-c3ccc4ccccc4c3)cc2)c2cc3ccccc3c3ccccc23)c([2H])c([2H])c1-c1cc2ccccc2c2ccccc12] 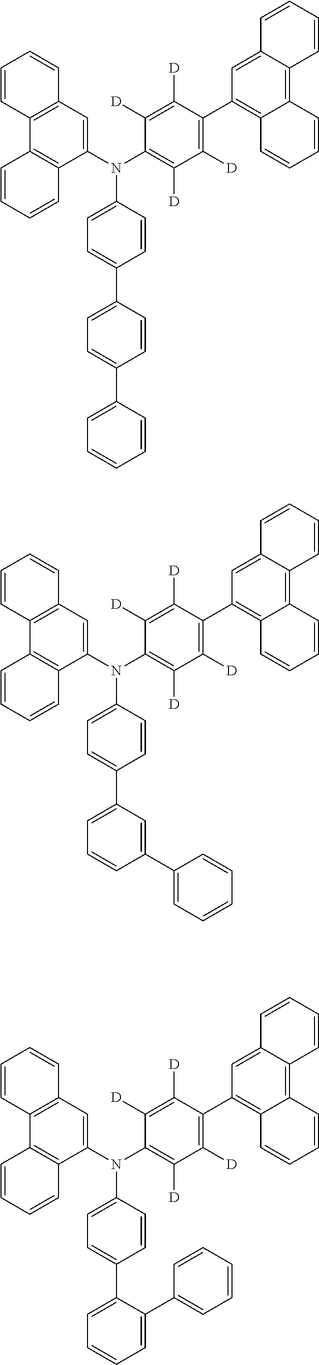 [SMILES: [2H]c1c([2H])c(N(c2ccc(-c3ccc(-c4ccccc4)cc3)cc2)c2cc3ccccc3c3ccccc23)c([2H])c([2H])c1-c1cc2ccccc2c2ccccc12 ; [2H]c1c([2H])c(N(c2ccc(-c3cccc(-c4ccccc4)c3)cc2)c2cc3ccccc3c3ccccc23)c([2H])c([2H])c1-c1cc2ccccc2c2ccccc12 ; [2H]c1c([2H])c(N(c2ccc(-c3ccccc3-c3ccccc3)cc2)c2cc3ccccc3c3ccccc23)c([2H])c([2H])c1-c1cc2ccccc2c2ccccc12] 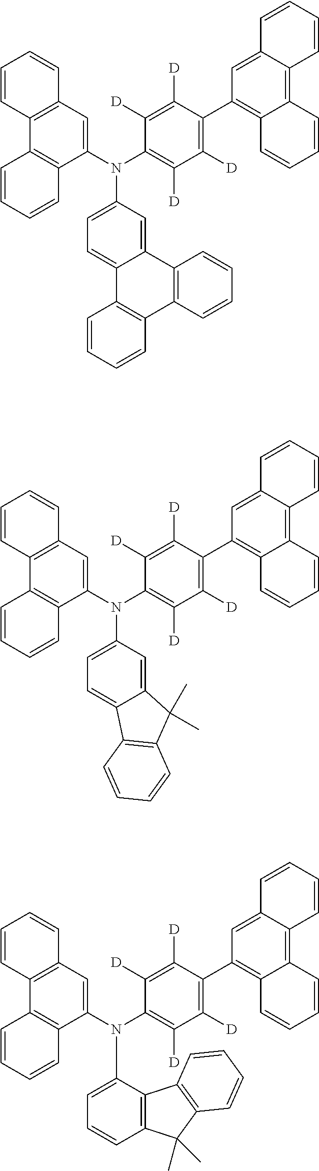 [SMILES: [2H]c1c([2H])c(N(c2ccc3c4ccccc4c4ccccc4c3c2)c2cc3ccccc3c3ccccc23)c([2H])c([2H])c1-c1cc2ccccc2c2ccccc12 ; [2H]c1c([2H])c(N(c2ccc3c(c2)C(C)(C)c2ccccc2-3)c2cc3ccccc3c3ccccc23)c([2H])c([2H])c1-c1cc2ccccc2c2ccccc12 ; [2H]c1c([2H])c(N(c2cccc3c2-c2ccccc2C3(C)C)c2cc3ccccc3c3ccccc23)c([2H])c([2H])c1-c1cc2ccccc2c2ccccc12] 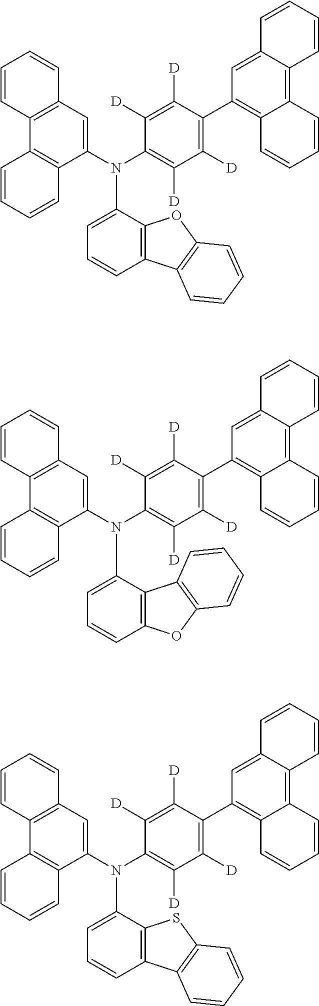 [SMILES: [2H]c1c([2H])c(N(c2cc3ccccc3c3ccccc23)c2cccc3c2oc2ccccc23)c([2H])c([2H])c1-c1cc2ccccc2c2ccccc12 ; [2H]c1c([2H])c(N(c2cc3ccccc3c3ccccc23)c2cccc3oc4ccccc4c23)c([2H])c([2H])c1-c1cc2ccccc2c2ccccc12 ; [2H]c1c([2H])c(N(c2cc3ccccc3c3ccccc23)c2cccc3c2sc2ccccc23)c([2H])c([2H])c1-c1cc2ccccc2c2ccccc12] 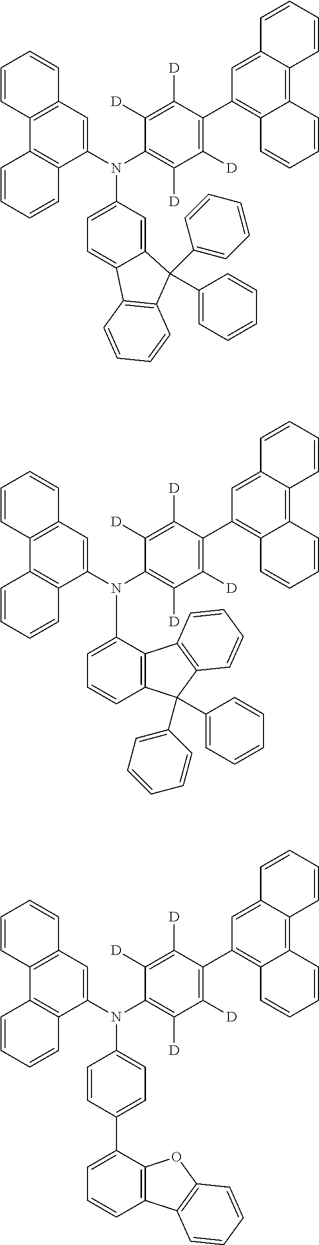 [SMILES: [2H]c1c([2H])c(N(c2ccc3c(c2)C(c2ccccc2)(c2ccccc2)c2ccccc2-3)c2cc3ccccc3c3ccccc23)c([2H])c([2H])c1-c1cc2ccccc2c2ccccc12 ; [2H]c1c([2H])c(N(c2cccc3c2-c2ccccc2C3(c2ccccc2)c2ccccc2)c2cc3ccccc3c3ccccc23)c([2H])c([2H])c1-c1cc2ccccc2c2ccccc12 ; [2H]c1c([2H])c(N(c2ccc(-c3cccc4c3oc3ccccc34)cc2)c2cc3ccccc3c3ccccc23)c([2H])c([2H])c1-c1cc2ccccc2c2ccccc12] 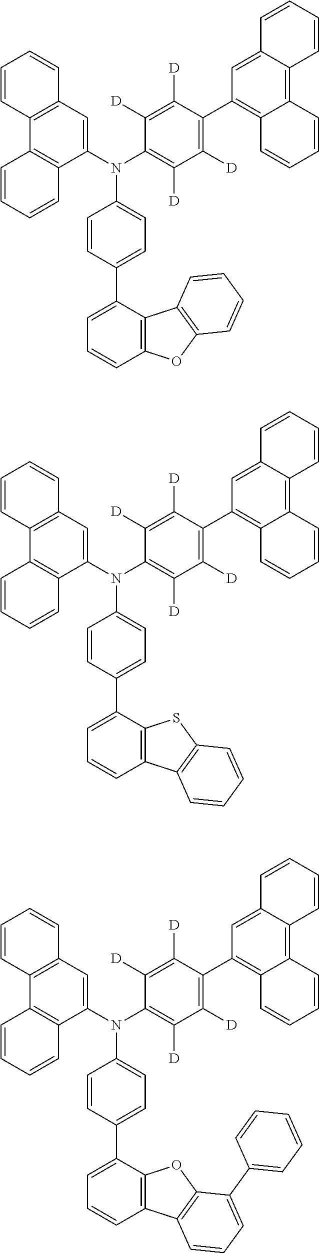 [SMILES: [2H]c1c([2H])c(N(c2ccc(-c3cccc4oc5ccccc5c34)cc2)c2cc3ccccc3c3ccccc23)c([2H])c([2H])c1-c1cc2ccccc2c2ccccc12 ; [2H]c1c([2H])c(N(c2ccc(-c3cccc4c3sc3ccccc34)cc2)c2cc3ccccc3c3ccccc23)c([2H])c([2H])c1-c1cc2ccccc2c2ccccc12 ; [2H]c1c([2H])c(N(c2ccc(-c3cccc4c3oc3c(-c5ccccc5)cccc34)cc2)c2cc3ccccc3c3ccccc23)c([2H])c([2H])c1-c1cc2ccccc2c2ccccc12] 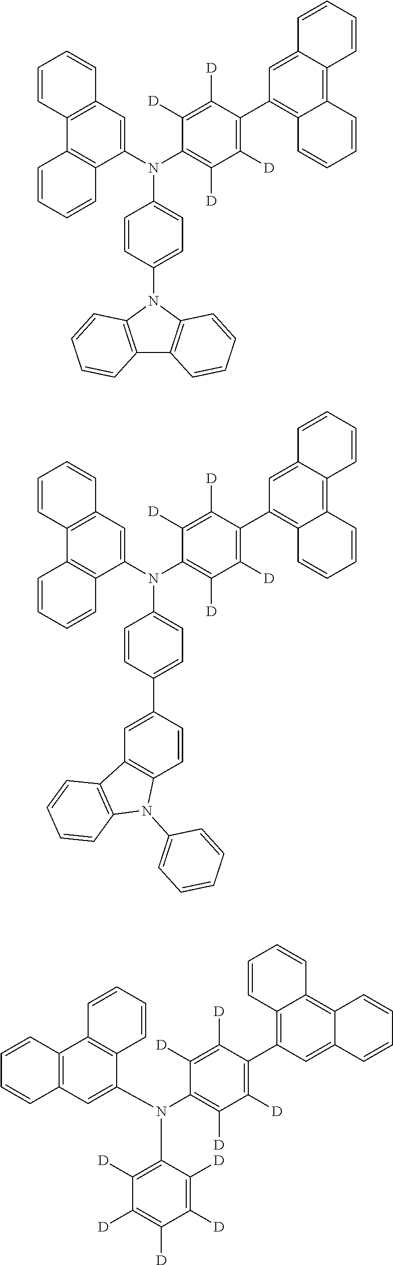 [SMILES: [2H]c1c([2H])c(N(c2ccc(-n3c4ccccc4c4ccccc43)cc2)c2cc3ccccc3c3ccccc23)c([2H])c([2H])c1-c1cc2ccccc2c2ccccc12 ; [2H]c1c([2H])c(N(c2ccc(-c3ccc4c(c3)c3ccccc3n4-c3ccccc3)cc2)c2cc3ccccc3c3ccccc23)c([2H])c([2H])c1-c1cc2ccccc2c2ccccc12 ; [2H]c1c([2H])c([2H])c(N(c2c([2H])c([2H])c(-c3cc4ccccc4c4ccccc34)c([2H])c2[2H])c2cc3ccccc3c3ccccc23)c([2H])c1[2H]] 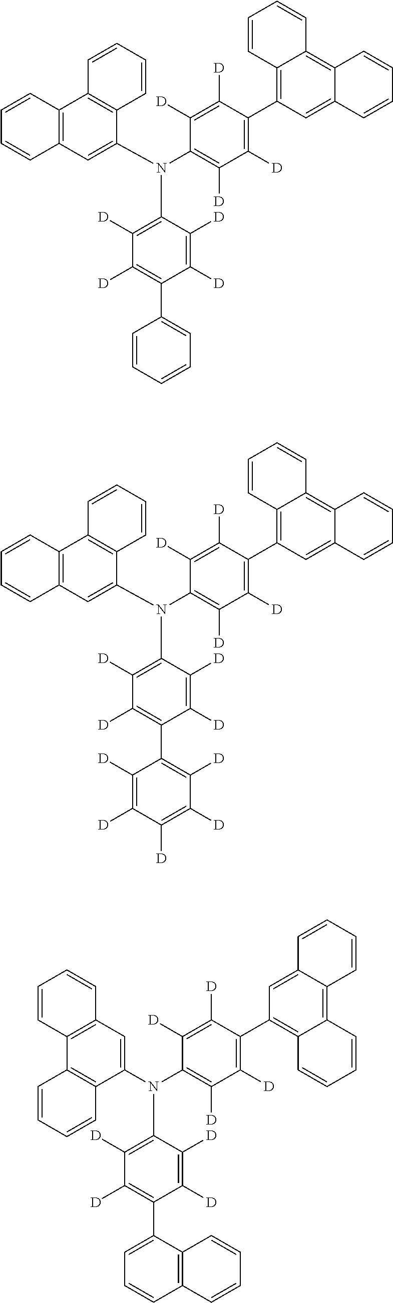 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1 ; [2H]c1c([2H])c([2H])c(-c2c([2H])c([2H])c(N(c3c([2H])c([2H])c(-c4cc5ccccc5c5ccccc45)c([2H])c3[2H])c3cc4ccccc4c4ccccc34)c([2H])c2[2H])c([2H])c1[2H] ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc2ccccc12] 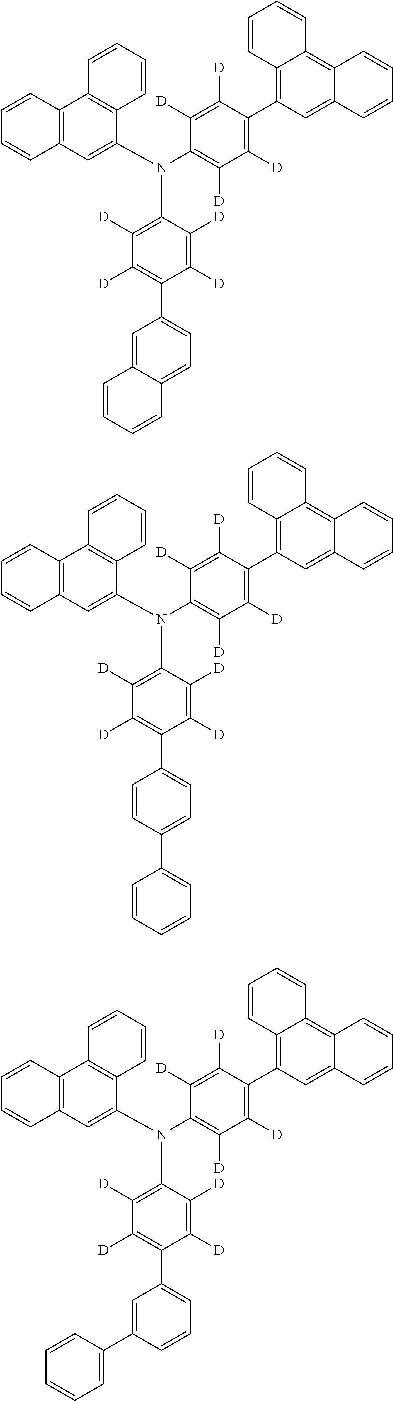 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc2ccccc2c1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc(-c2ccccc2)cc1 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cccc(-c2ccccc2)c1] 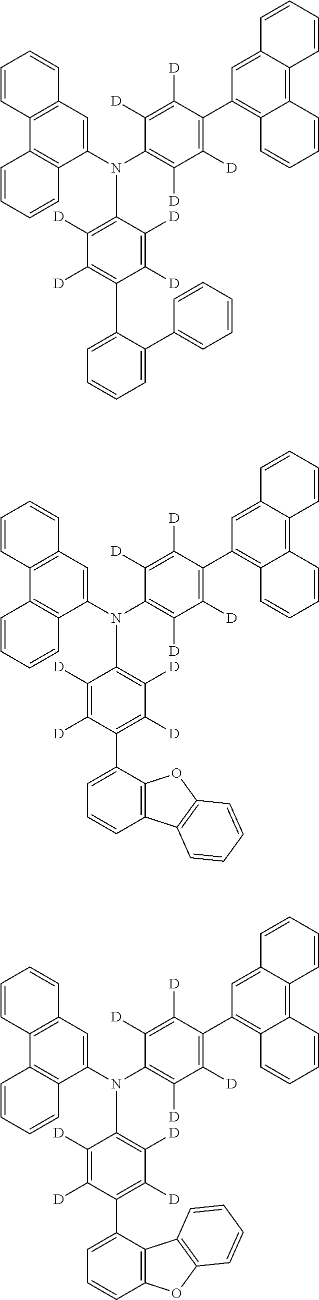 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccccc1-c1ccccc1 ; [2H]c1c([2H])c(N(c2c([2H])c([2H])c(-c3cccc4c3oc3ccccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4oc5ccccc5c34)c([2H])c2[2H])c2cc3ccccc3c3ccccc23)c([2H])c([2H])c1-c1cc2ccccc2c2ccccc12] 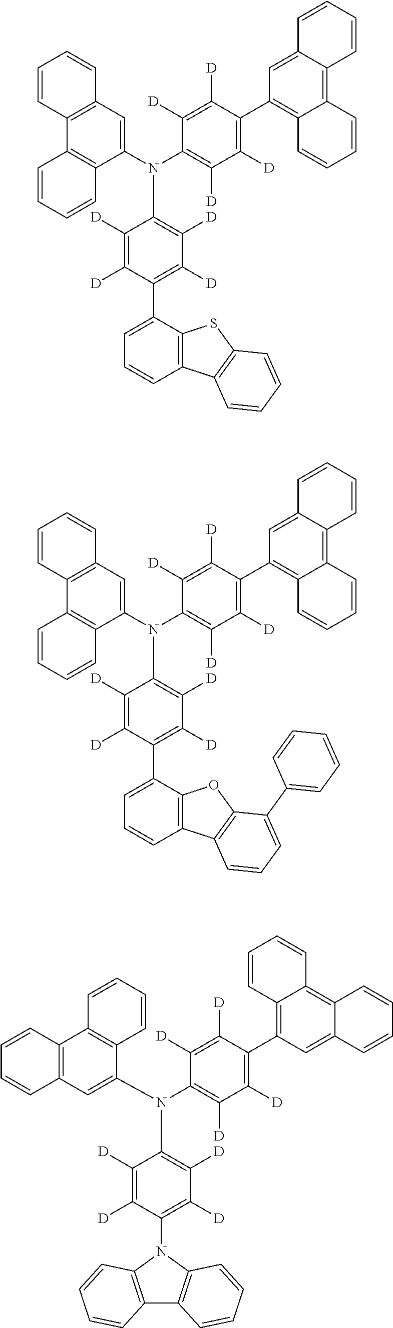 [SMILES: [2H]c1c([2H])c(N(c2c([2H])c([2H])c(-c3cccc4c3sc3ccccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-c3cccc4c3oc3c(-c5ccccc5)cccc34)c([2H])c2[2H])c2cc3ccccc3c3ccccc23)c([2H])c([2H])c1-c1cc2ccccc2c2ccccc12 ; [2H]c1c([2H])c(N(c2c([2H])c([2H])c(-n3c4ccccc4c4ccccc43)c([2H])c2[2H])c2cc3ccccc3c3ccccc23)c([2H])c([2H])c1-c1cc2ccccc2c2ccccc12] 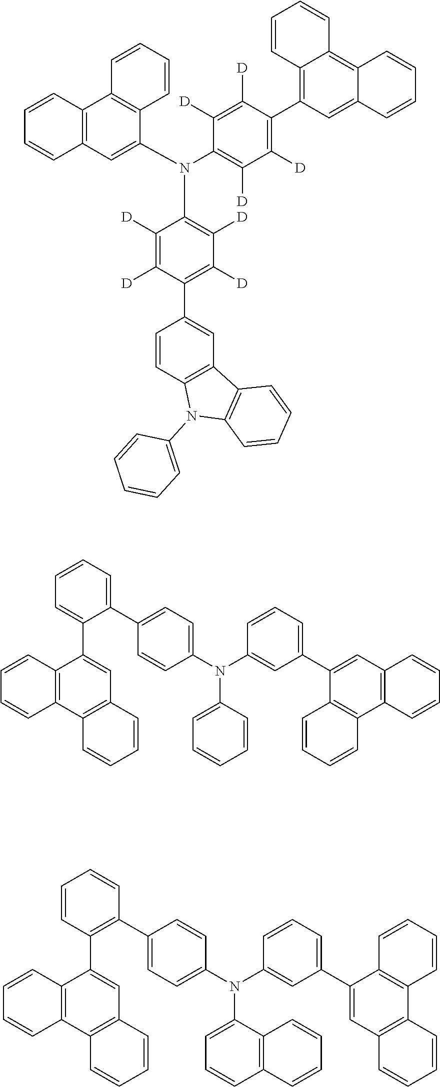 [SMILES: [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1ccc2c(c1)c1ccccc1n2-c1ccccc1 ; c1ccc(N(c2ccc(-c3ccccc3-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3ccccc3-c3cc4ccccc4c4ccccc34)cc2)c2cccc3ccccc23)c1] 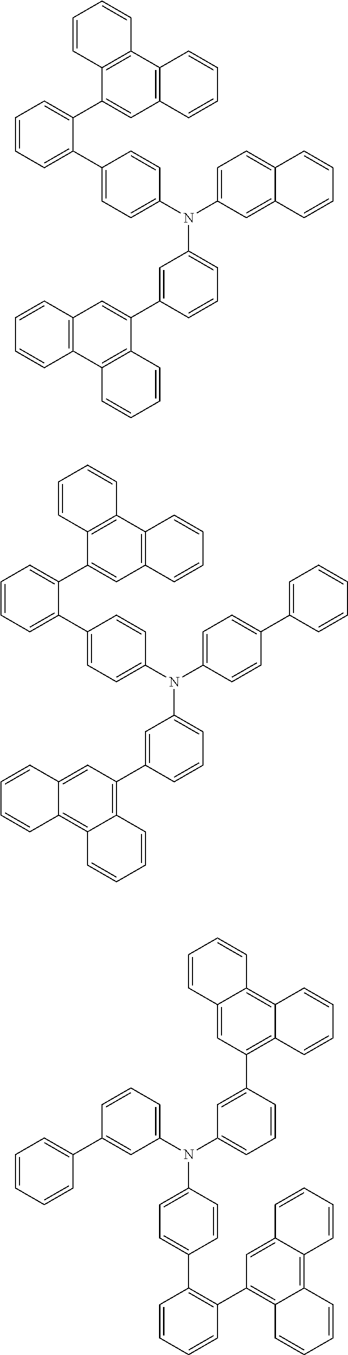 [SMILES: c1cc(-c2cc3ccccc3c3ccccc23)cc(N(c2ccc(-c3ccccc3-c3cc4ccccc4c4ccccc34)cc2)c2ccc3ccccc3c2)c1 ; c1ccc(-c2ccc(N(c3ccc(-c4ccccc4-c4cc5ccccc5c5ccccc45)cc3)c3cccc(-c4cc5ccccc5c5ccccc45)c3)cc2)cc1 ; c1ccc(-c2cccc(N(c3ccc(-c4ccccc4-c4cc5ccccc5c5ccccc45)cc3)c3cccc(-c4cc5ccccc5c5ccccc45)c3)c2)cc1] 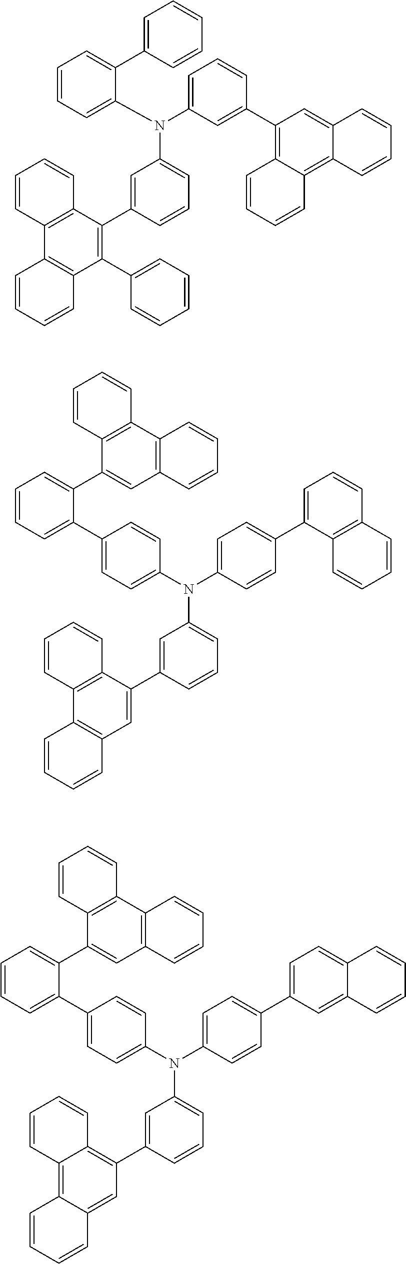 [SMILES: c1ccc(-c2ccccc2N(c2cccc(-c3cc4ccccc4c4ccccc34)c2)c2cccc(-c3c(-c4ccccc4)c4ccccc4c4ccccc34)c2)cc1 ; c1cc(-c2cc3ccccc3c3ccccc23)cc(N(c2ccc(-c3ccccc3-c3cc4ccccc4c4ccccc34)cc2)c2ccc(-c3cccc4ccccc34)cc2)c1 ; c1cc(-c2cc3ccccc3c3ccccc23)cc(N(c2ccc(-c3ccc4ccccc4c3)cc2)c2ccc(-c3ccccc3-c3cc4ccccc4c4ccccc34)cc2)c1] 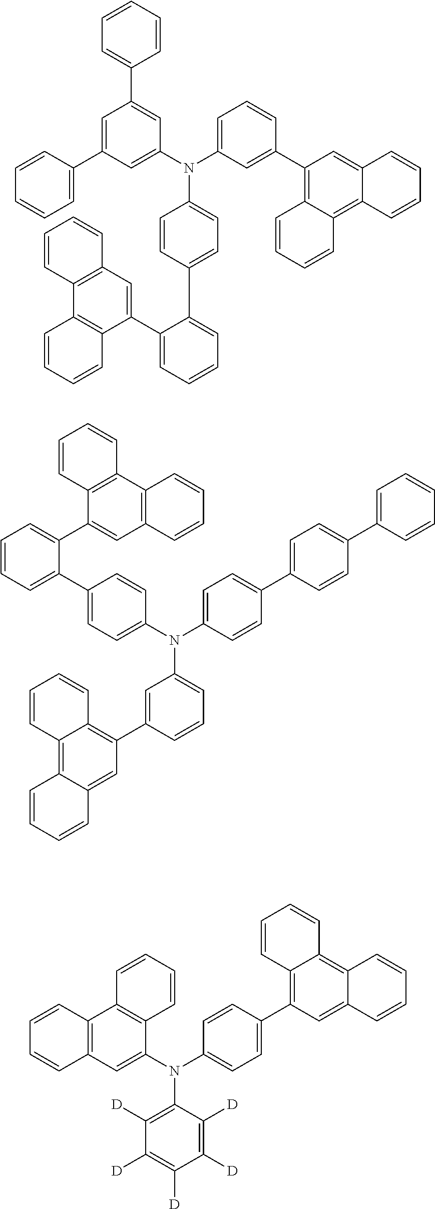 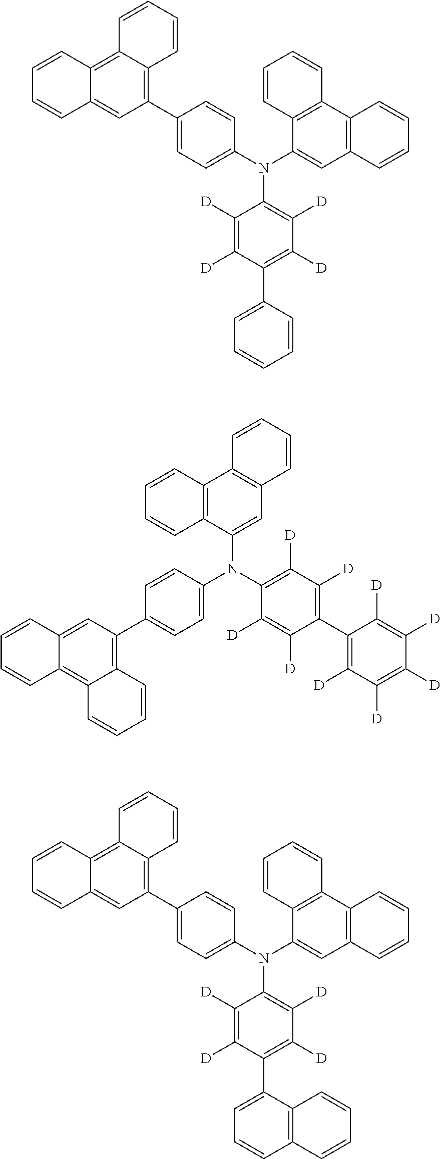 [SMILES: [2H]c1c([2H])c(N(c2ccc(-c3cc4ccccc4c4ccccc34)cc2)c2cc3ccccc3c3ccccc23)c([2H])c([2H])c1-c1ccccc1 ; [2H]c1c([2H])c([2H])c(-c2c([2H])c([2H])c(N(c3ccc(-c4cc5ccccc5c5ccccc45)cc3)c3cc4ccccc4c4ccccc34)c([2H])c2[2H])c([2H])c1[2H] ; [2H]c1c([2H])c(N(c2ccc(-c3cc4ccccc4c4ccccc34)cc2)c2cc3ccccc3c3ccccc23)c([2H])c([2H])c1-c1cccc2ccccc12] 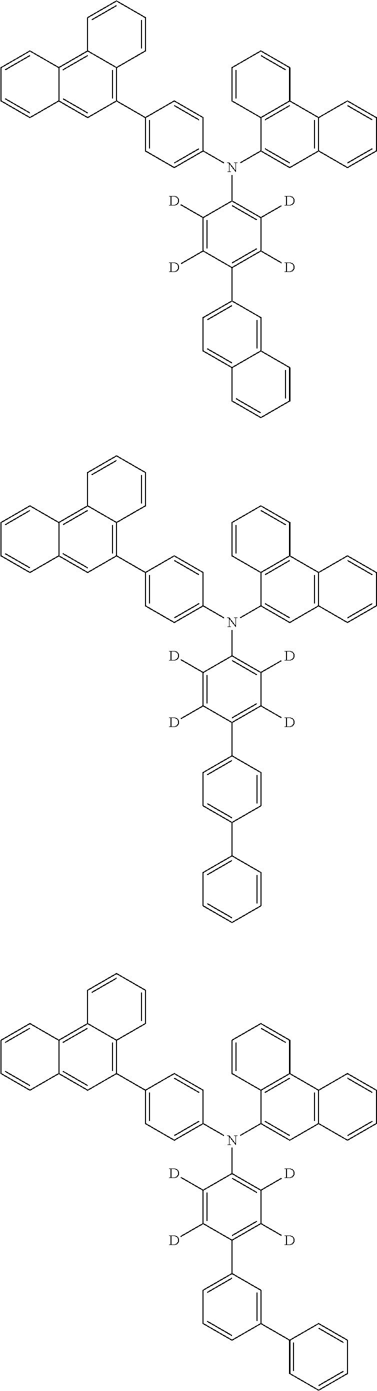 [SMILES: [2H]c1c([2H])c(N(c2ccc(-c3cc4ccccc4c4ccccc34)cc2)c2cc3ccccc3c3ccccc23)c([2H])c([2H])c1-c1ccc2ccccc2c1 ; [2H]c1c([2H])c(N(c2ccc(-c3cc4ccccc4c4ccccc34)cc2)c2cc3ccccc3c3ccccc23)c([2H])c([2H])c1-c1ccc(-c2ccccc2)cc1 ; [2H]c1c([2H])c(N(c2ccc(-c3cc4ccccc4c4ccccc34)cc2)c2cc3ccccc3c3ccccc23)c([2H])c([2H])c1-c1cccc(-c2ccccc2)c1] 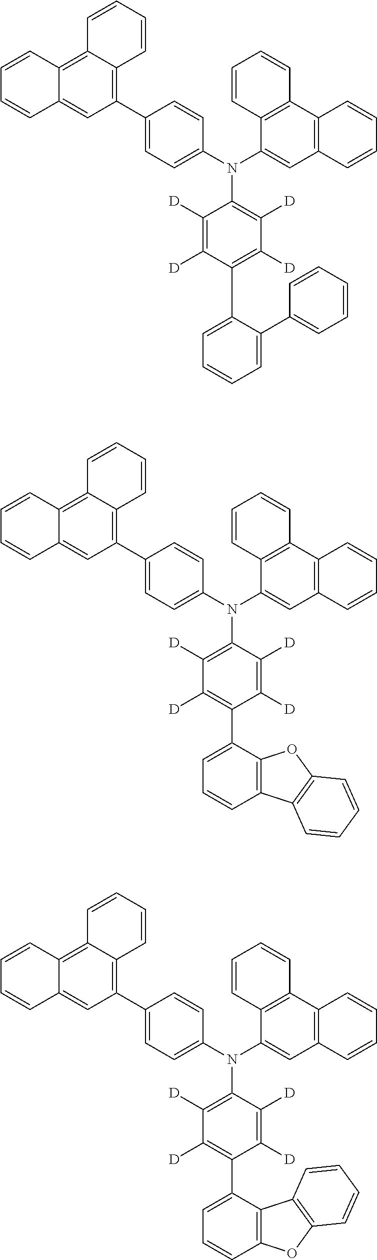 [SMILES: [2H]c1c([2H])c(N(c2ccc(-c3cc4ccccc4c4ccccc34)cc2)c2cc3ccccc3c3ccccc23)c([2H])c([2H])c1-c1ccccc1-c1ccccc1 ; [2H]c1c([2H])c(N(c2ccc(-c3cc4ccccc4c4ccccc34)cc2)c2cc3ccccc3c3ccccc23)c([2H])c([2H])c1-c1cccc2c1oc1ccccc12 ; [2H]c1c([2H])c(N(c2ccc(-c3cc4ccccc4c4ccccc34)cc2)c2cc3ccccc3c3ccccc23)c([2H])c([2H])c1-c1cccc2oc3ccccc3c12] 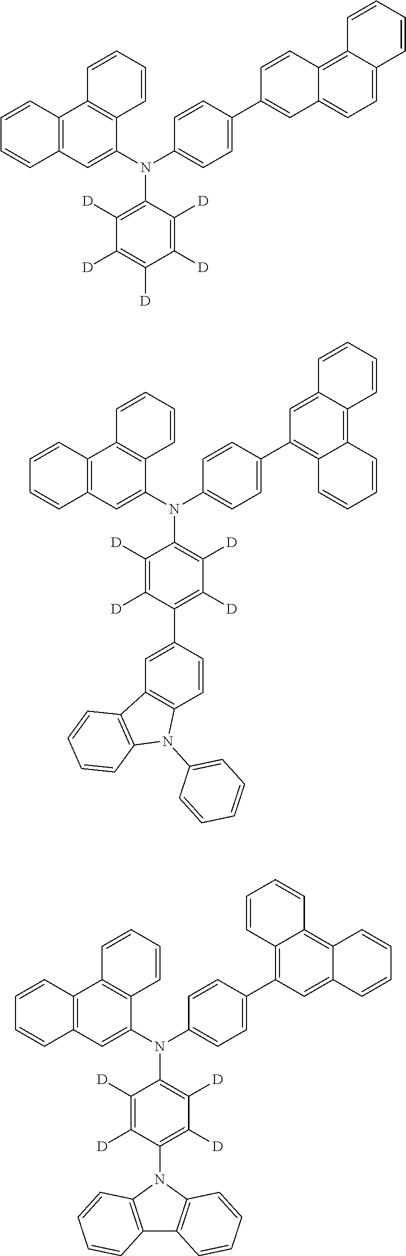 [SMILES: [2H]c1c([2H])c([2H])c(N(c2ccc(-c3ccc4c(ccc5ccccc54)c3)cc2)c2cc3ccccc3c3ccccc23)c([2H])c1[2H] ; [2H]c1c([2H])c(N(c2ccc(-c3cc4ccccc4c4ccccc34)cc2)c2cc3ccccc3c3ccccc23)c([2H])c([2H])c1-c1ccc2c(c1)c1ccccc1n2-c1ccccc1 ; [2H]c1c([2H])c(-n2c3ccccc3c3ccccc32)c([2H])c([2H])c1N(c1ccc(-c2cc3ccccc3c3ccccc23)cc1)c1cc2ccccc2c2ccccc12] 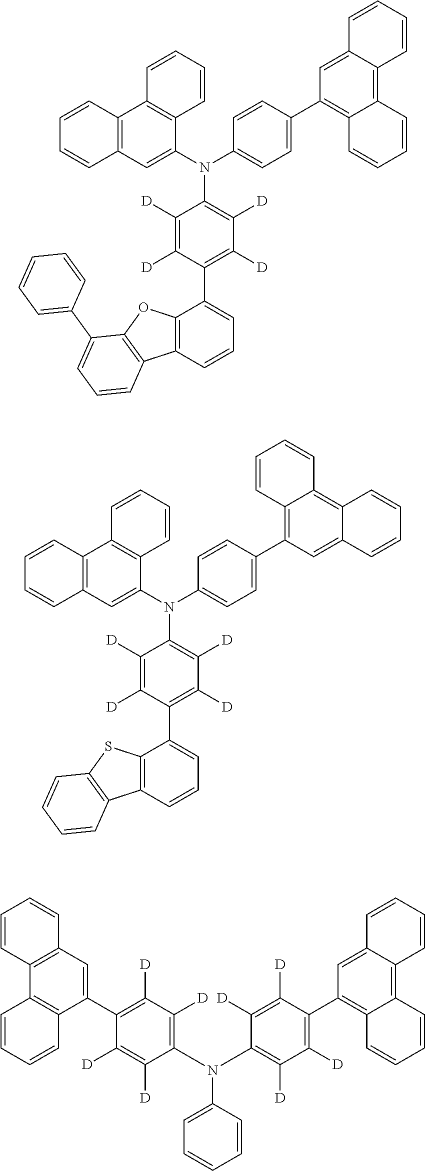 [SMILES: [2H]c1c([2H])c(N(c2ccc(-c3cc4ccccc4c4ccccc34)cc2)c2cc3ccccc3c3ccccc23)c([2H])c([2H])c1-c1cccc2c1oc1c(-c3ccccc3)cccc12 ; [2H]c1c([2H])c(N(c2ccc(-c3cc4ccccc4c4ccccc34)cc2)c2cc3ccccc3c3ccccc23)c([2H])c([2H])c1-c1cccc2c1sc1ccccc12 ; [2H]c1c([2H])c(N(c2ccccc2)c2c([2H])c([2H])c(-c3cc4ccccc4c4ccccc34)c([2H])c2[2H])c([2H])c([2H])c1-c1cc2ccccc2c2ccccc12] 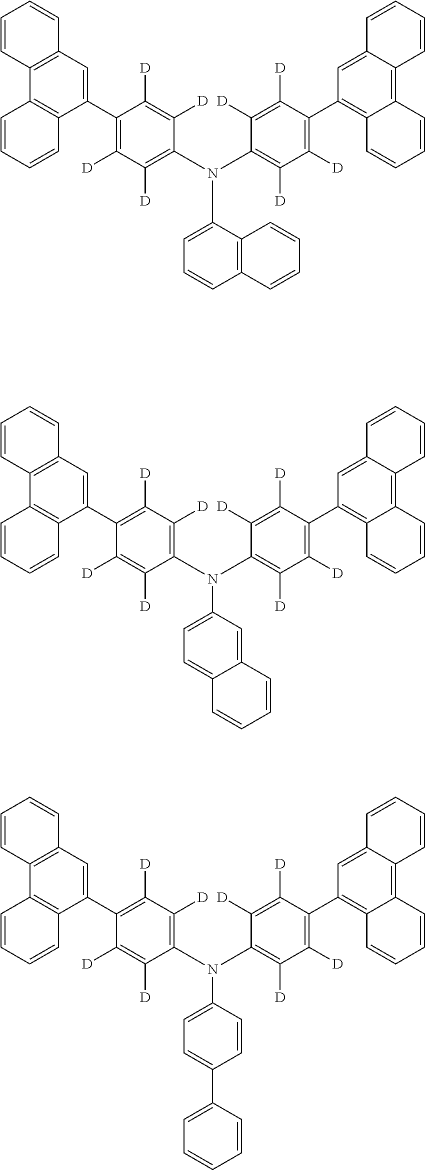 [SMILES: [2H]c1c([2H])c(N(c2c([2H])c([2H])c(-c3cc4ccccc4c4ccccc34)c([2H])c2[2H])c2cccc3ccccc23)c([2H])c([2H])c1-c1cc2ccccc2c2ccccc12 ; [2H]c1c([2H])c(N(c2ccc3ccccc3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccccc3)cc2)c2c([2H])c([2H])c(-c3cc4ccccc4c4ccccc34)c([2H])c2[2H])c([2H])c([2H])c1-c1cc2ccccc2c2ccccc12] 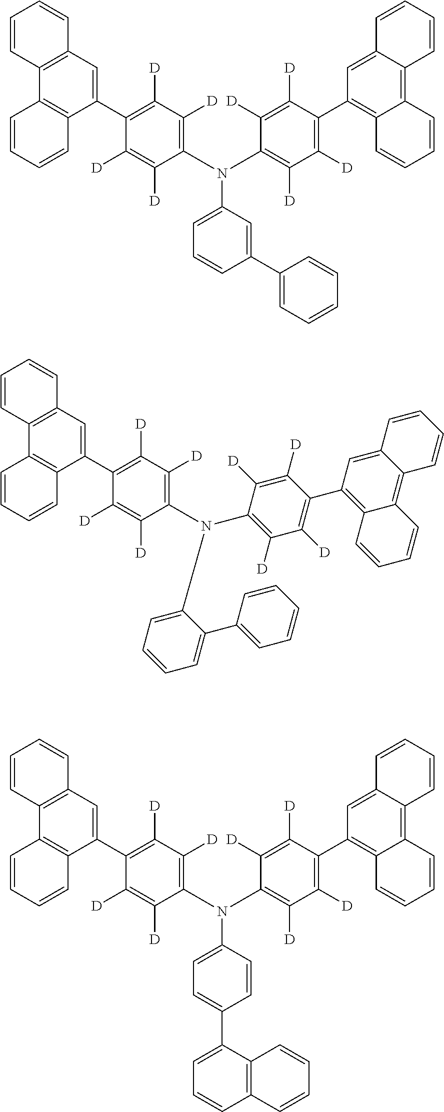 [SMILES: [2H]c1c([2H])c(N(c2cccc(-c3ccccc3)c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccccc2-c2ccccc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc4ccccc34)cc2)c2c([2H])c([2H])c(-c3cc4ccccc4c4ccccc34)c([2H])c2[2H])c([2H])c([2H])c1-c1cc2ccccc2c2ccccc12] 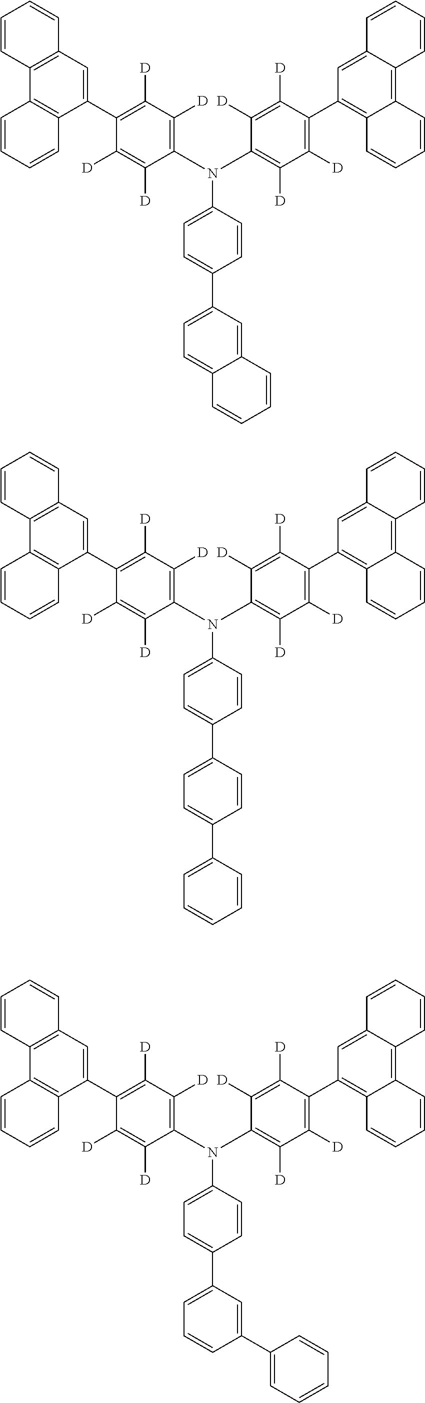 [SMILES: [2H]c1c([2H])c(N(c2ccc(-c3ccc4ccccc4c3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3ccc(-c4ccccc4)cc3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc(-c3cccc(-c4ccccc4)c3)cc2)c2c([2H])c([2H])c(-c3cc4ccccc4c4ccccc34)c([2H])c2[2H])c([2H])c([2H])c1-c1cc2ccccc2c2ccccc12] 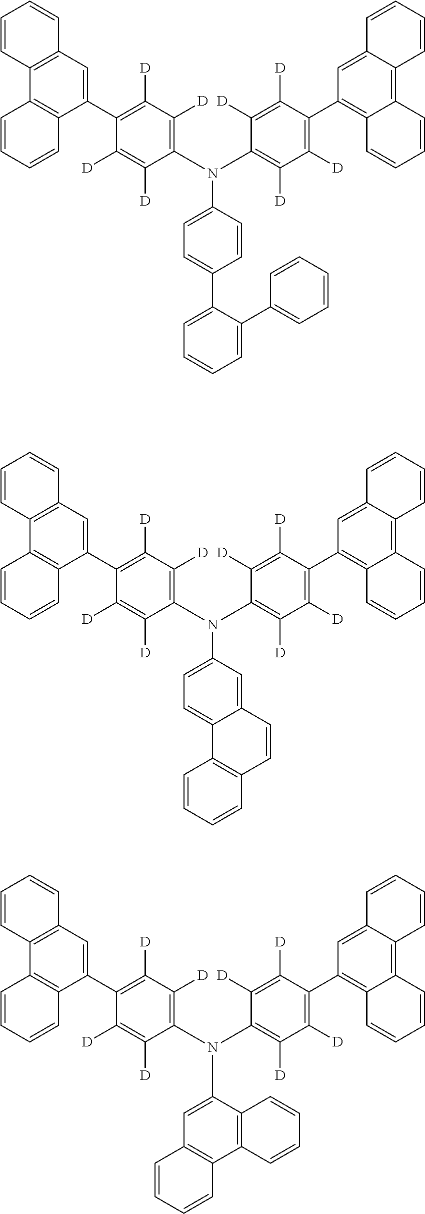 [SMILES: [2H]c1c([2H])c(N(c2ccc(-c3ccccc3-c3ccccc3)cc2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc3c(ccc4ccccc43)c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2c([2H])c([2H])c(-c3cc4ccccc4c4ccccc34)c([2H])c2[2H])c2cc3ccccc3c3ccccc23)c([2H])c([2H])c1-c1cc2ccccc2c2ccccc12] 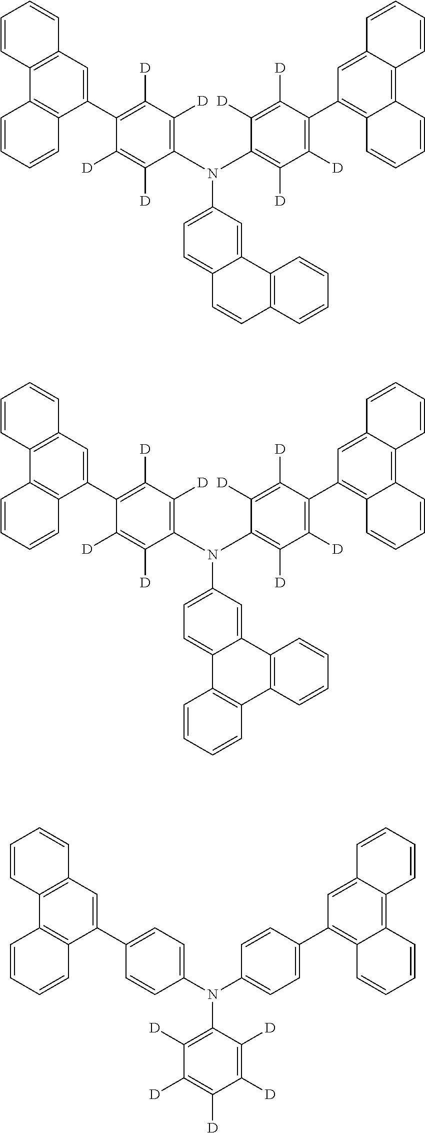 [SMILES: [2H]c1c([2H])c(N(c2ccc3ccc4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c(N(c2ccc3c4ccccc4c4ccccc4c3c2)c2c([2H])c([2H])c(-c3cc4ccccc4c4ccccc34)c([2H])c2[2H])c([2H])c([2H])c1-c1cc2ccccc2c2ccccc12 ; [2H]c1c([2H])c([2H])c(N(c2ccc(-c3cc4ccccc4c4ccccc34)cc2)c2ccc(-c3cc4ccccc4c4ccccc34)cc2)c([2H])c1[2H]] 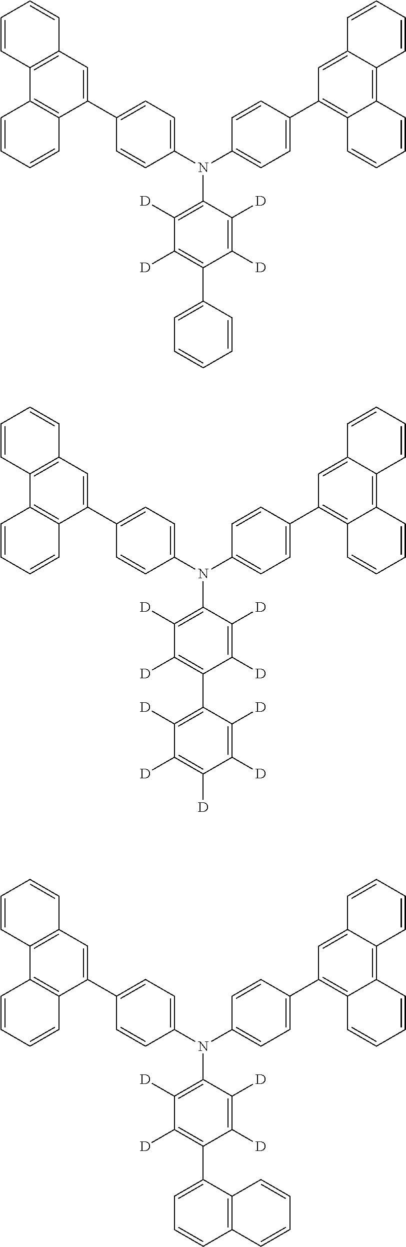 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1 ; [2H]c1c([2H])c([2H])c(-c2c([2H])c([2H])c(N(c3ccc(-c4cc5ccccc5c5ccccc45)cc3)c3ccc(-c4cc5ccccc5c5ccccc45)cc3)c([2H])c2[2H])c([2H])c1[2H] ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1cccc2ccccc12] 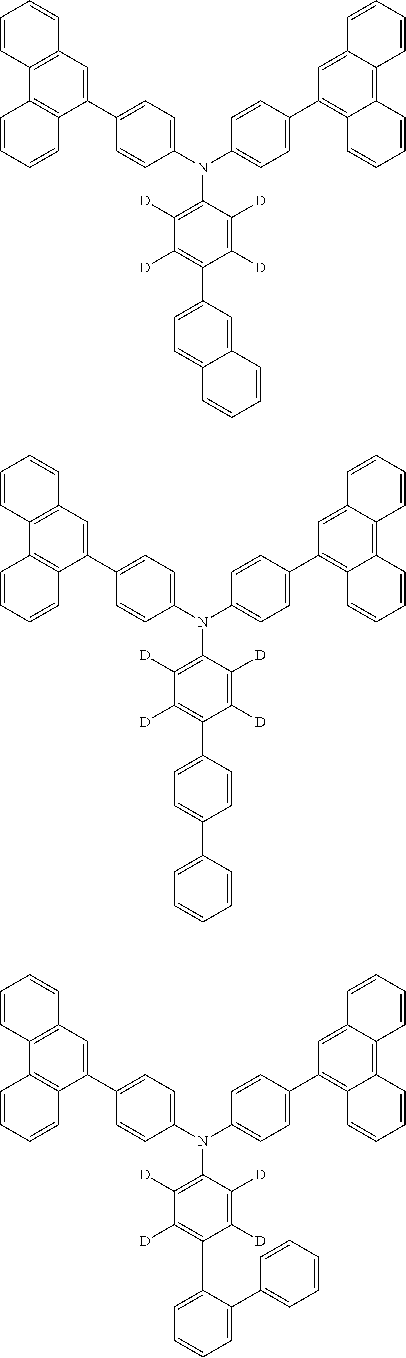 [SMILES: [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc2ccccc2c1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccc(-c2ccccc2)cc1 ; [2H]c1c([2H])c(N(c2ccc(-c3cc4ccccc4c4ccccc34)cc2)c2ccc(-c3cc4ccccc4c4ccccc34)cc2)c([2H])c([2H])c1-c1ccccc1-c1ccccc1] 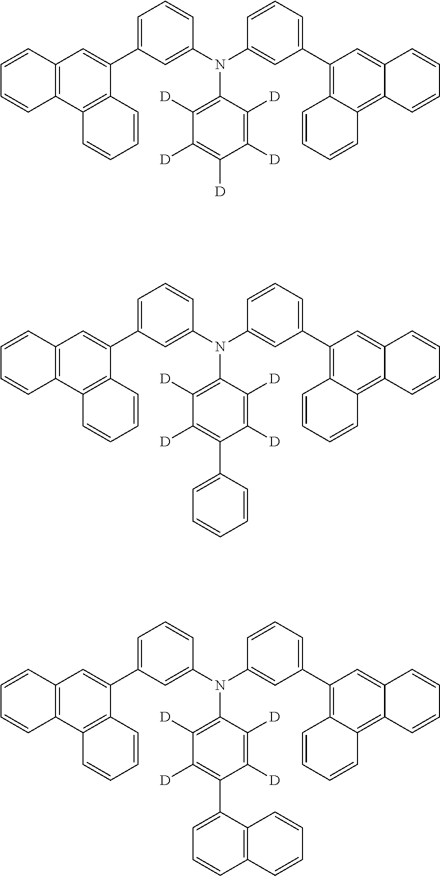 [SMILES: [2H]c1c([2H])c([2H])c(N(c2cccc(-c3cc4ccccc4c4ccccc34)c2)c2cccc(-c3cc4ccccc4c4ccccc34)c2)c([2H])c1[2H] ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccccc1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1cccc2ccccc12] 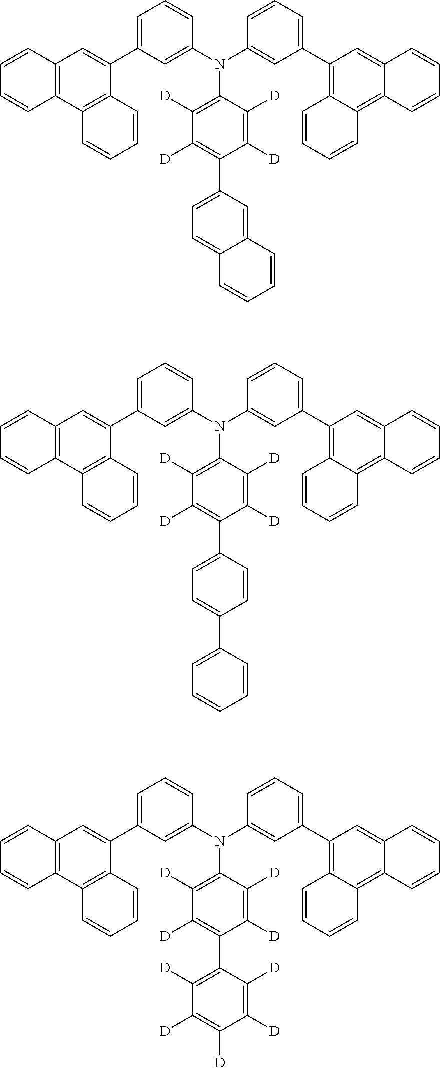 [SMILES: [2H]c1c([2H])c(N(c2cccc(-c3cc4ccccc4c4ccccc34)c2)c2cccc(-c3cc4ccccc4c4ccccc34)c2)c([2H])c([2H])c1-c1ccc2ccccc2c1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccc(-c2ccccc2)cc1 ; [2H]c1c([2H])c([2H])c(-c2c([2H])c([2H])c(N(c3cccc(-c4cc5ccccc5c5ccccc45)c3)c3cccc(-c4cc5ccccc5c5ccccc45)c3)c([2H])c2[2H])c([2H])c1[2H]] 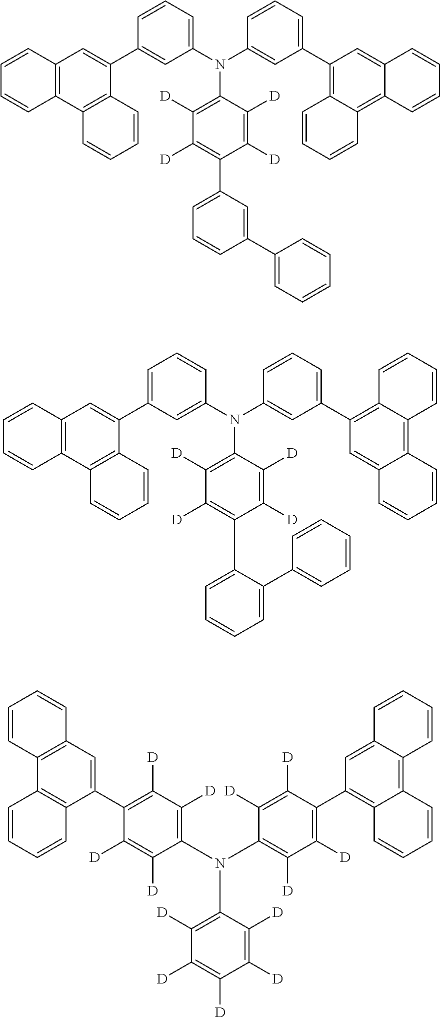 [SMILES: [2H]c1c([2H])c(N(c2cccc(-c3cc4ccccc4c4ccccc34)c2)c2cccc(-c3cc4ccccc4c4ccccc34)c2)c([2H])c([2H])c1-c1cccc(-c2ccccc2)c1 ; [2H]c1c([2H])c(N(c2cccc(-c3cc4ccccc4c4ccccc34)c2)c2cccc(-c3cc4ccccc4c4ccccc34)c2)c([2H])c([2H])c1-c1ccccc1-c1ccccc1 ; [2H]c1c([2H])c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c1[2H]] 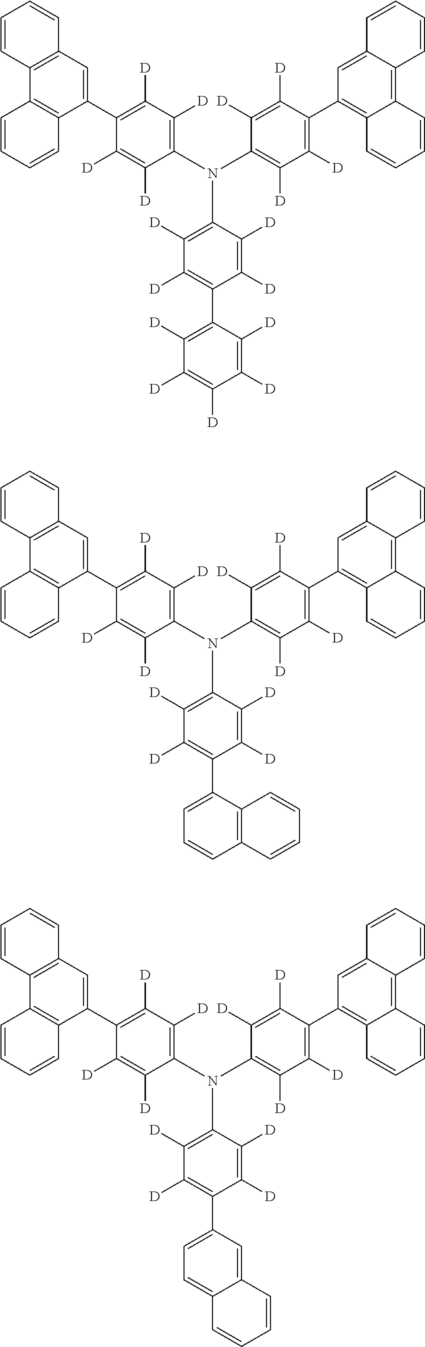 [SMILES: [2H]c1c([2H])c([2H])c(-c2c([2H])c([2H])c(N(c3c([2H])c([2H])c(-c4cc5ccccc5c5ccccc45)c([2H])c3[2H])c3c([2H])c([2H])c(-c4cc5ccccc5c5ccccc45)c([2H])c3[2H])c([2H])c2[2H])c([2H])c1[2H] ; [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1cccc2ccccc12 ; [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1ccc2ccccc2c1] 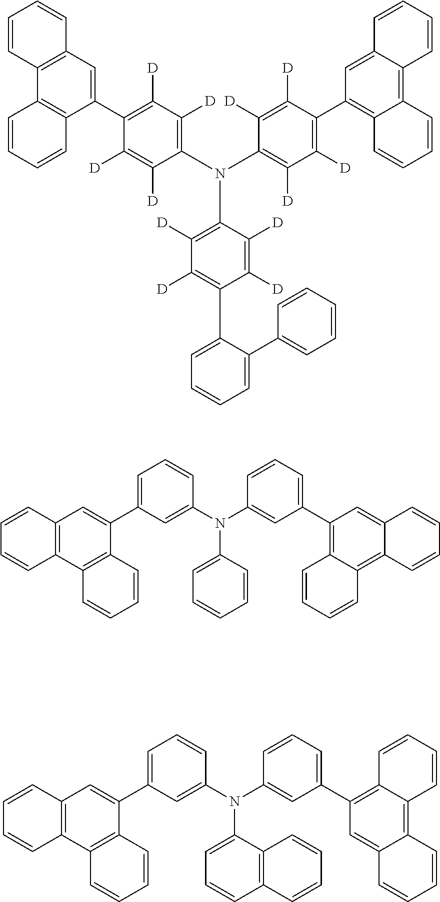 [SMILES: [2H]c1c([2H])c(N(c2c([2H])c([2H])c(-c3cc4ccccc4c4ccccc34)c([2H])c2[2H])c2c([2H])c([2H])c(-c3cc4ccccc4c4ccccc34)c([2H])c2[2H])c([2H])c([2H])c1-c1ccccc1-c1ccccc1 ; c1ccc(N(c2cccc(-c3cc4ccccc4c4ccccc34)c2)c2cccc(-c3cc4ccccc4c4ccccc34)c2)cc1 ; c1cc(-c2cc3ccccc3c3ccccc23)cc(N(c2cccc(-c3cc4ccccc4c4ccccc34)c2)c2cccc3ccccc23)c1] 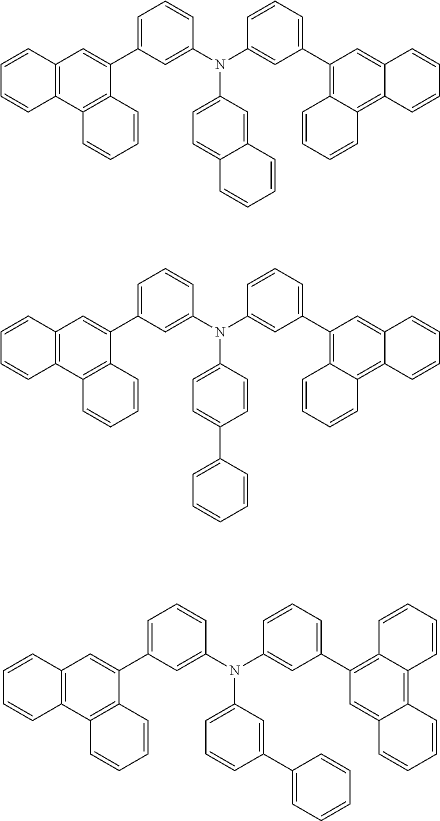 [SMILES: c1cc(-c2cc3ccccc3c3ccccc23)cc(N(c2cccc(-c3cc4ccccc4c4ccccc34)c2)c2ccc3ccccc3c2)c1 ; c1ccc(-c2ccc(N(c3cccc(-c4cc5ccccc5c5ccccc45)c3)c3cccc(-c4cc5ccccc5c5ccccc45)c3)cc2)cc1 ; c1ccc(-c2cccc(N(c3cccc(-c4cc5ccccc5c5ccccc45)c3)c3cccc(-c4cc5ccccc5c5ccccc45)c3)c2)cc1] 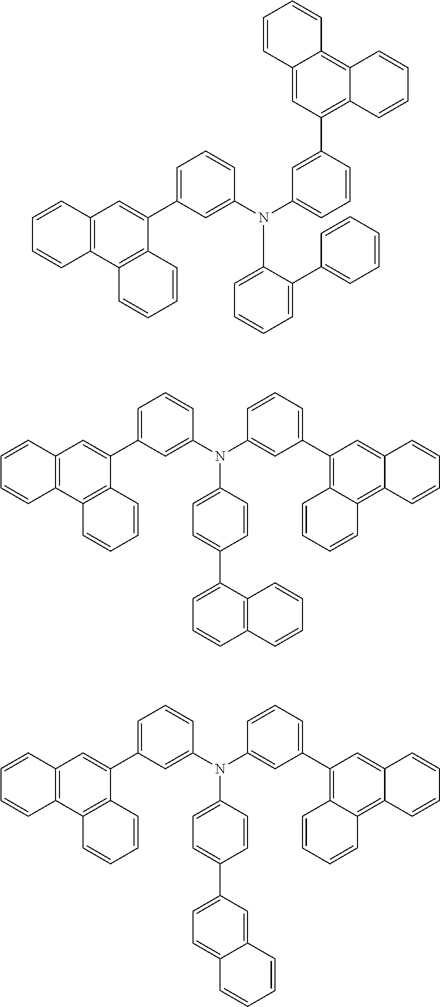 [SMILES: c1ccc(-c2ccccc2N(c2cccc(-c3cc4ccccc4c4ccccc34)c2)c2cccc(-c3cc4ccccc4c4ccccc34)c2)cc1 ; c1cc(-c2cc3ccccc3c3ccccc23)cc(N(c2ccc(-c3cccc4ccccc34)cc2)c2cccc(-c3cc4ccccc4c4ccccc34)c2)c1 ; c1cc(-c2cc3ccccc3c3ccccc23)cc(N(c2ccc(-c3ccc4ccccc4c3)cc2)c2cccc(-c3cc4ccccc4c4ccccc34)c2)c1] 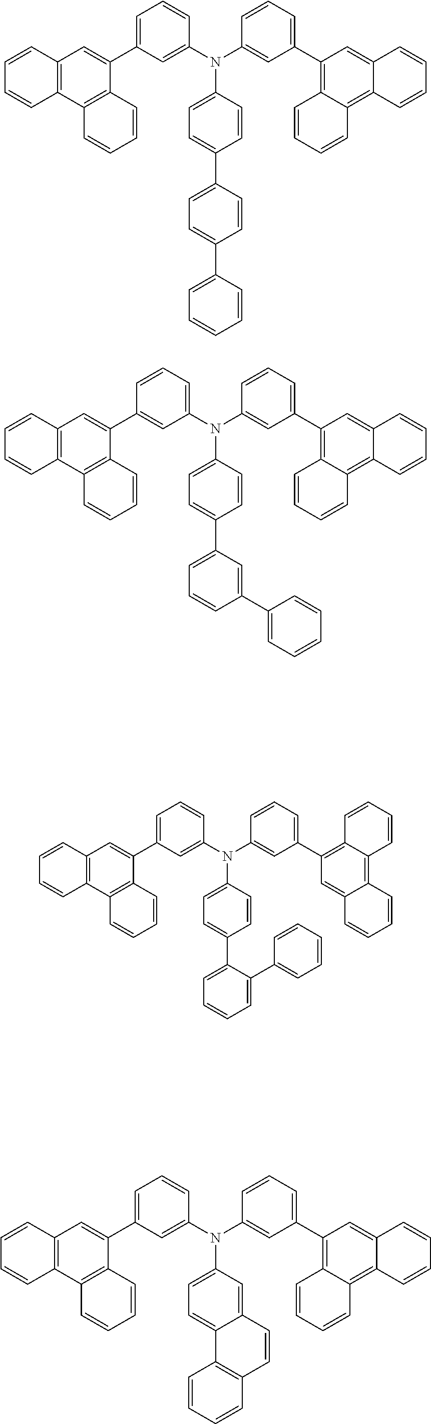 [SMILES: c1ccc(-c2ccc(-c3ccc(N(c4cccc(-c5cc6ccccc6c6ccccc56)c4)c4cccc(-c5cc6ccccc6c6ccccc56)c4)cc3)cc2)cc1 ; c1ccc(-c2cccc(-c3ccc(N(c4cccc(-c5cc6ccccc6c6ccccc56)c4)c4cccc(-c5cc6ccccc6c6ccccc56)c4)cc3)c2)cc1 ; c1ccc(-c2ccccc2-c2ccc(N(c3cccc(-c4cc5ccccc5c5ccccc45)c3)c3cccc(-c4cc5ccccc5c5ccccc45)c3)cc2)cc1 ; c1cc(-c2cc3ccccc3c3ccccc23)cc(N(c2cccc(-c3cc4ccccc4c4ccccc34)c2)c2ccc3c(ccc4ccccc43)c2)c1] 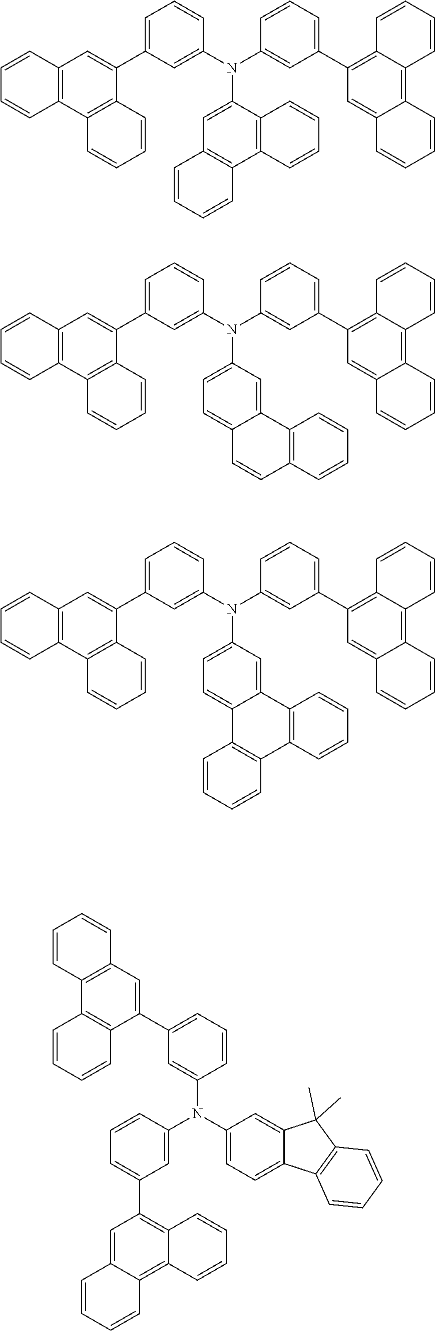 [SMILES: c1cc(-c2cc3ccccc3c3ccccc23)cc(N(c2cccc(-c3cc4ccccc4c4ccccc34)c2)c2cc3ccccc3c3ccccc23)c1 ; c1cc(-c2cc3ccccc3c3ccccc23)cc(N(c2cccc(-c3cc4ccccc4c4ccccc34)c2)c2ccc3ccc4ccccc4c3c2)c1 ; c1cc(-c2cc3ccccc3c3ccccc23)cc(N(c2cccc(-c3cc4ccccc4c4ccccc34)c2)c2ccc3c4ccccc4c4ccccc4c3c2)c1 ; CC1(C)c2ccccc2-c2ccc(N(c3cccc(-c4cc5ccccc5c5ccccc45)c3)c3cccc(-c4cc5ccccc5c5ccccc45)c3)cc21] 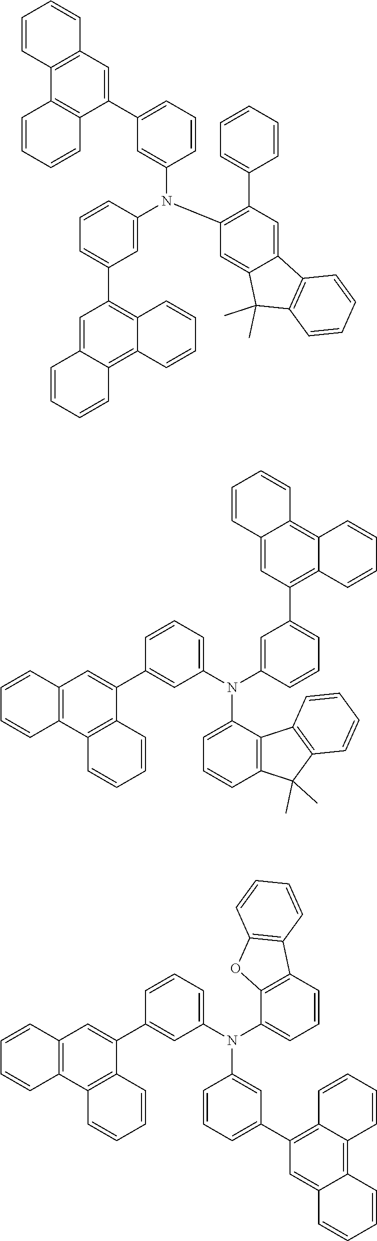 [SMILES: CC1(C)c2ccccc2-c2cc(-c3ccccc3)c(N(c3cccc(-c4cc5ccccc5c5ccccc45)c3)c3cccc(-c4cc5ccccc5c5ccccc45)c3)cc21 ; CC1(C)c2ccccc2-c2c(N(c3cccc(-c4cc5ccccc5c5ccccc45)c3)c3cccc(-c4cc5ccccc5c5ccccc45)c3)cccc21 ; c1cc(-c2cc3ccccc3c3ccccc23)cc(N(c2cccc(-c3cc4ccccc4c4ccccc34)c2)c2cccc3c2oc2ccccc23)c1] 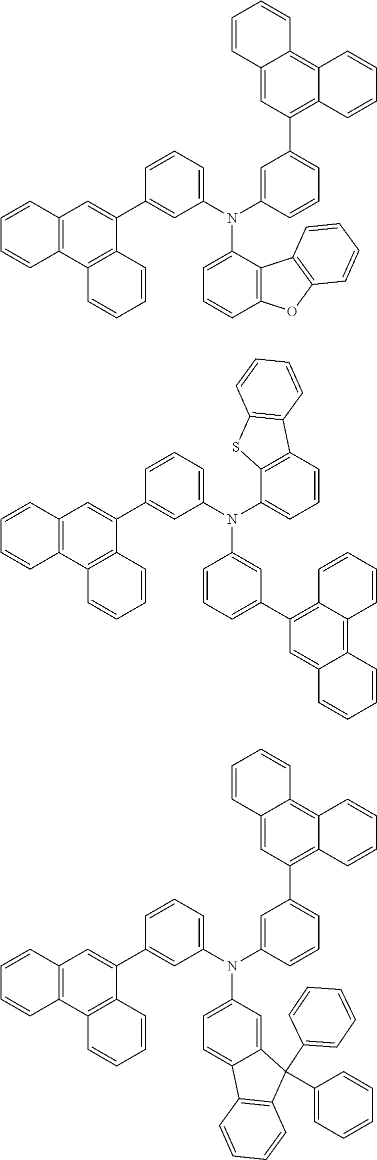 [SMILES: c1cc(-c2cc3ccccc3c3ccccc23)cc(N(c2cccc(-c3cc4ccccc4c4ccccc34)c2)c2cccc3oc4ccccc4c23)c1 ; c1cc(-c2cc3ccccc3c3ccccc23)cc(N(c2cccc(-c3cc4ccccc4c4ccccc34)c2)c2cccc3c2sc2ccccc23)c1 ; c1ccc(C2(c3ccccc3)c3ccccc3-c3ccc(N(c4cccc(-c5cc6ccccc6c6ccccc56)c4)c4cccc(-c5cc6ccccc6c6ccccc56)c4)cc32)cc1] 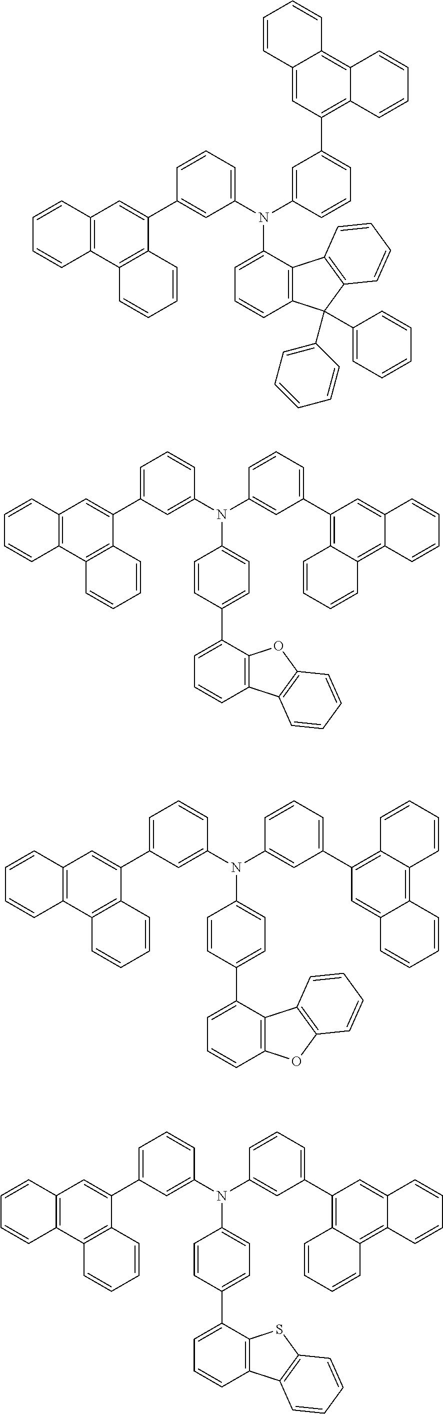 [SMILES: c1ccc(C2(c3ccccc3)c3ccccc3-c3c(N(c4cccc(-c5cc6ccccc6c6ccccc56)c4)c4cccc(-c5cc6ccccc6c6ccccc56)c4)cccc32)cc1 ; c1cc(-c2cc3ccccc3c3ccccc23)cc(N(c2ccc(-c3cccc4c3oc3ccccc34)cc2)c2cccc(-c3cc4ccccc4c4ccccc34)c2)c1 ; c1cc(-c2cc3ccccc3c3ccccc23)cc(N(c2ccc(-c3cccc4oc5ccccc5c34)cc2)c2cccc(-c3cc4ccccc4c4ccccc34)c2)c1 ; c1cc(-c2cc3ccccc3c3ccccc23)cc(N(c2ccc(-c3cccc4c3sc3ccccc34)cc2)c2cccc(-c3cc4ccccc4c4ccccc34)c2)c1] 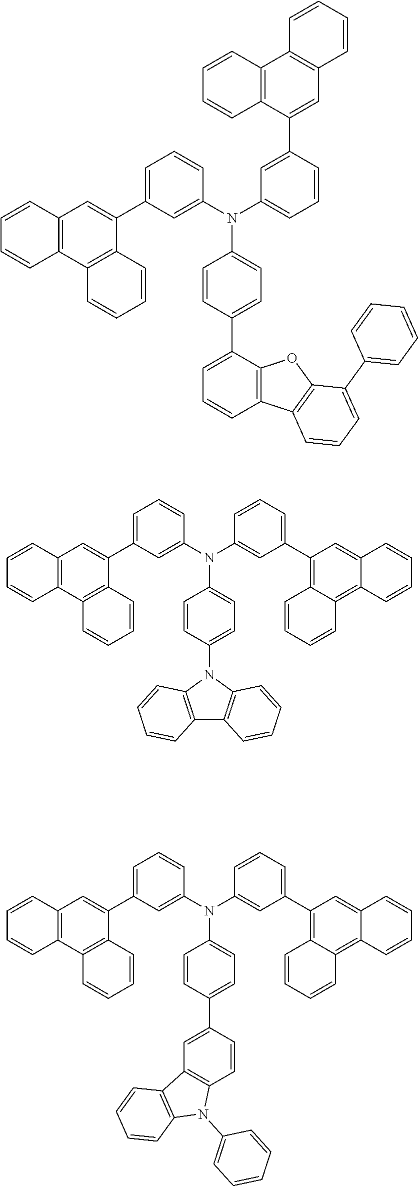 [SMILES: c1ccc(-c2cccc3c2oc2c(-c4ccc(N(c5cccc(-c6cc7ccccc7c7ccccc67)c5)c5cccc(-c6cc7ccccc7c7ccccc67)c5)cc4)cccc23)cc1 ; c1cc(-c2cc3ccccc3c3ccccc23)cc(N(c2ccc(-n3c4ccccc4c4ccccc43)cc2)c2cccc(-c3cc4ccccc4c4ccccc34)c2)c1 ; c1ccc(-n2c3ccccc3c3cc(-c4ccc(N(c5cccc(-c6cc7ccccc7c7ccccc67)c5)c5cccc(-c6cc7ccccc7c7ccccc67)c5)cc4)ccc32)cc1] 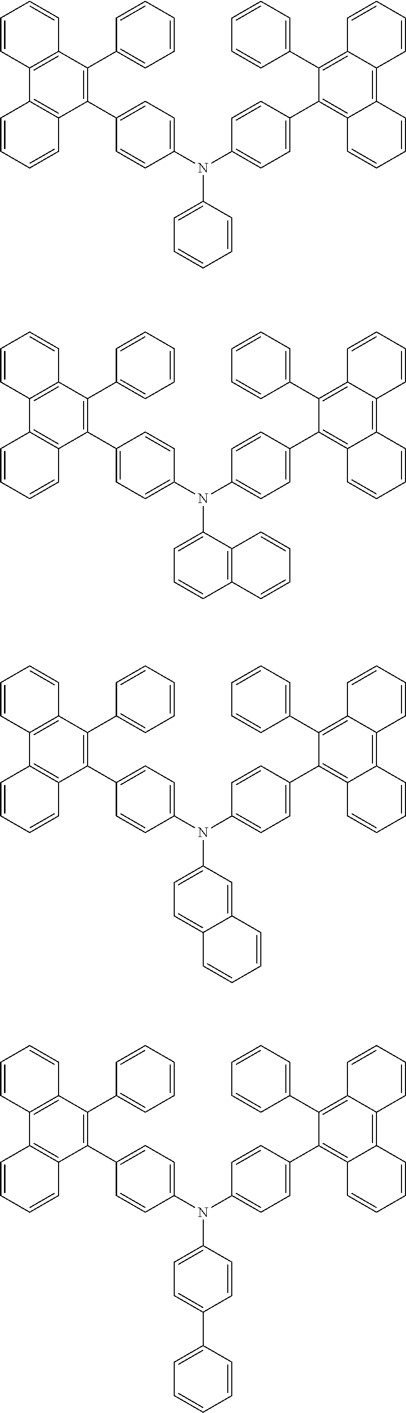 [SMILES: c1ccc(-c2c(-c3ccc(N(c4ccccc4)c4ccc(-c5c(-c6ccccc6)c6ccccc6c6ccccc56)cc4)cc3)c3ccccc3c3ccccc23)cc1 ; c1ccc(-c2c(-c3ccc(N(c4ccc(-c5c(-c6ccccc6)c6ccccc6c6ccccc56)cc4)c4cccc5ccccc45)cc3)c3ccccc3c3ccccc23)cc1 ; c1ccc(-c2c(-c3ccc(N(c4ccc(-c5c(-c6ccccc6)c6ccccc6c6ccccc56)cc4)c4ccc5ccccc5c4)cc3)c3ccccc3c3ccccc23)cc1 ; c1ccc(-c2ccc(N(c3ccc(-c4c(-c5ccccc5)c5ccccc5c5ccccc45)cc3)c3ccc(-c4c(-c5ccccc5)c5ccccc5c5ccccc45)cc3)cc2)cc1] 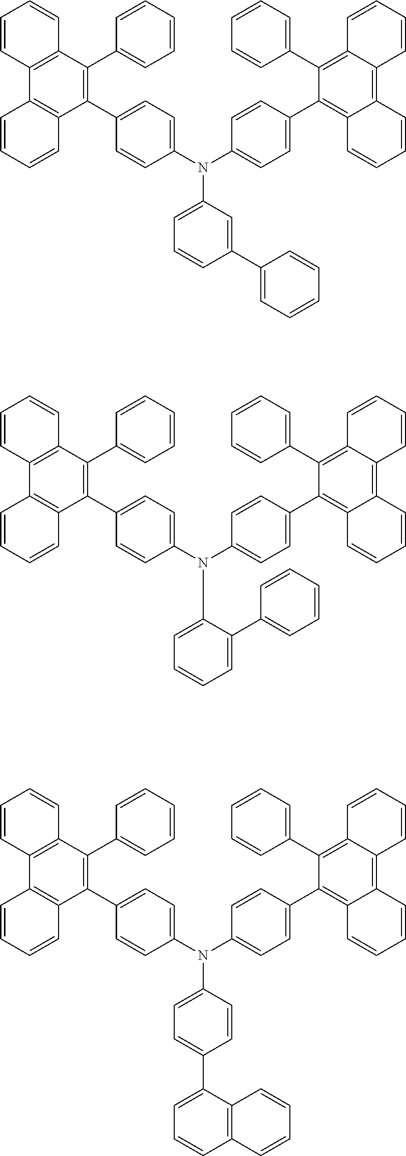 [SMILES: c1ccc(-c2cccc(N(c3ccc(-c4c(-c5ccccc5)c5ccccc5c5ccccc45)cc3)c3ccc(-c4c(-c5ccccc5)c5ccccc5c5ccccc45)cc3)c2)cc1 ; c1ccc(-c2ccccc2N(c2ccc(-c3c(-c4ccccc4)c4ccccc4c4ccccc34)cc2)c2ccc(-c3c(-c4ccccc4)c4ccccc4c4ccccc34)cc2)cc1 ; c1ccc(-c2c(-c3ccc(N(c4ccc(-c5cccc6ccccc56)cc4)c4ccc(-c5c(-c6ccccc6)c6ccccc6c6ccccc56)cc4)cc3)c3ccccc3c3ccccc23)cc1] 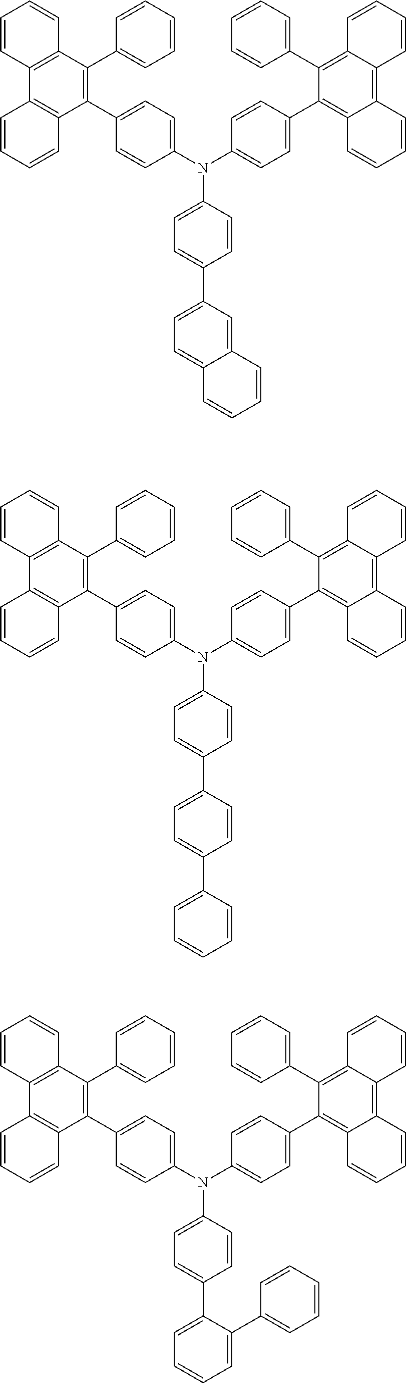 [SMILES: c1ccc(-c2c(-c3ccc(N(c4ccc(-c5ccc6ccccc6c5)cc4)c4ccc(-c5c(-c6ccccc6)c6ccccc6c6ccccc56)cc4)cc3)c3ccccc3c3ccccc23)cc1 ; c1ccc(-c2ccc(-c3ccc(N(c4ccc(-c5c(-c6ccccc6)c6ccccc6c6ccccc56)cc4)c4ccc(-c5c(-c6ccccc6)c6ccccc6c6ccccc56)cc4)cc3)cc2)cc1 ; c1ccc(-c2ccccc2-c2ccc(N(c3ccc(-c4c(-c5ccccc5)c5ccccc5c5ccccc45)cc3)c3ccc(-c4c(-c5ccccc5)c5ccccc5c5ccccc45)cc3)cc2)cc1] 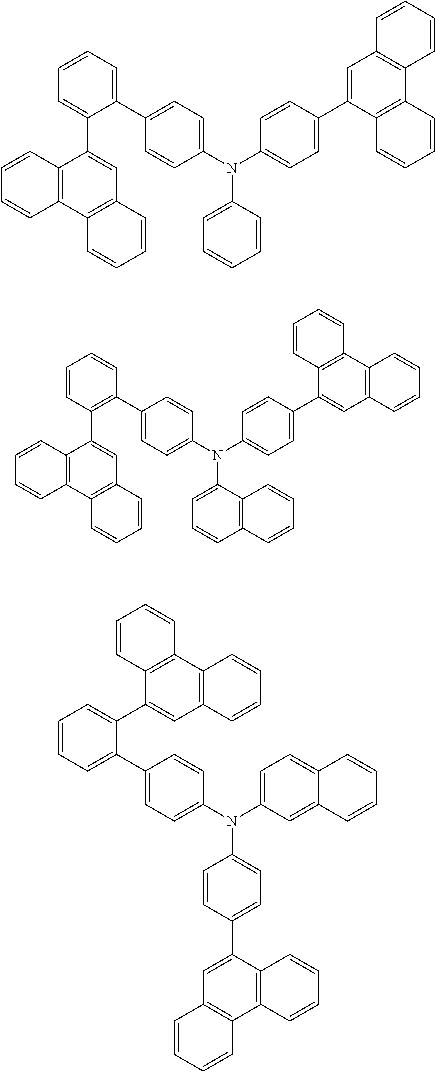 [SMILES: c1ccc(N(c2ccc(-c3ccccc3-c3cc4ccccc4c4ccccc34)cc2)c2ccc(-c3cc4ccccc4c4ccccc34)cc2)cc1 ; c1ccc(-c2cc3ccccc3c3ccccc23)c(-c2ccc(N(c3ccc(-c4cc5ccccc5c5ccccc45)cc3)c3cccc4ccccc34)cc2)c1 ; c1ccc(-c2cc3ccccc3c3ccccc23)c(-c2ccc(N(c3ccc(-c4cc5ccccc5c5ccccc45)cc3)c3ccc4ccccc4c3)cc2)c1] 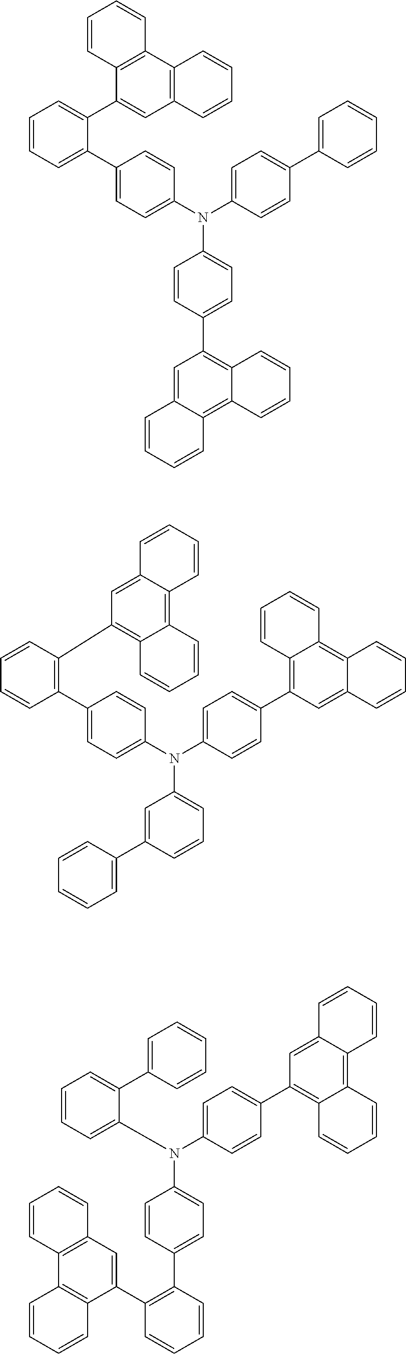 [SMILES: c1ccc(-c2ccc(N(c3ccc(-c4ccccc4-c4cc5ccccc5c5ccccc45)cc3)c3ccc(-c4cc5ccccc5c5ccccc45)cc3)cc2)cc1 ; c1ccc(-c2cccc(N(c3ccc(-c4ccccc4-c4cc5ccccc5c5ccccc45)cc3)c3ccc(-c4cc5ccccc5c5ccccc45)cc3)c2)cc1 ; c1ccc(-c2ccccc2N(c2ccc(-c3ccccc3-c3cc4ccccc4c4ccccc34)cc2)c2ccc(-c3cc4ccccc4c4ccccc34)cc2)cc1] 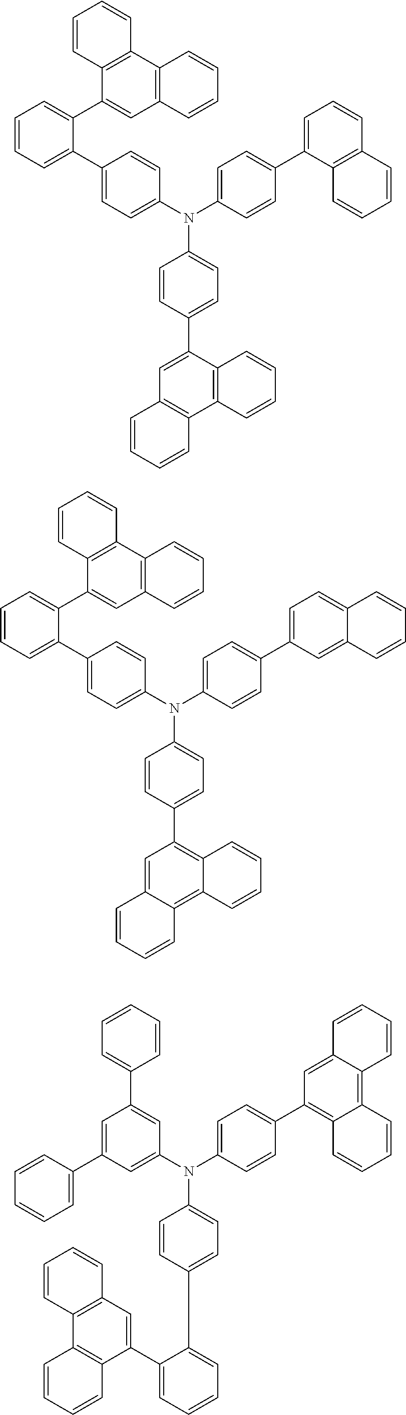 [SMILES: c1ccc(-c2cc3ccccc3c3ccccc23)c(-c2ccc(N(c3ccc(-c4cccc5ccccc45)cc3)c3ccc(-c4cc5ccccc5c5ccccc45)cc3)cc2)c1 ; c1ccc(-c2cc3ccccc3c3ccccc23)c(-c2ccc(N(c3ccc(-c4ccc5ccccc5c4)cc3)c3ccc(-c4cc5ccccc5c5ccccc45)cc3)cc2)c1 ; c1ccc(-c2cc(-c3ccccc3)cc(N(c3ccc(-c4ccccc4-c4cc5ccccc5c5ccccc45)cc3)c3ccc(-c4cc5ccccc5c5ccccc45)cc3)c2)cc1] 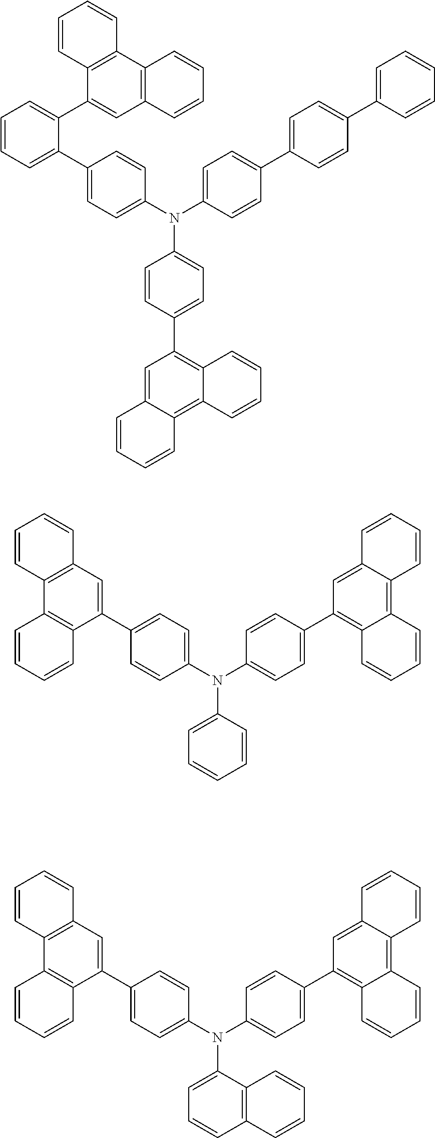 [SMILES: c1ccc(-c2ccc(-c3ccc(N(c4ccc(-c5ccccc5-c5cc6ccccc6c6ccccc56)cc4)c4ccc(-c5cc6ccccc6c6ccccc56)cc4)cc3)cc2)cc1 ; c1ccc(N(c2ccc(-c3cc4ccccc4c4ccccc34)cc2)c2ccc(-c3cc4ccccc4c4ccccc34)cc2)cc1 ; c1ccc2c(N(c3ccc(-c4cc5ccccc5c5ccccc45)cc3)c3ccc(-c4cc5ccccc5c5ccccc45)cc3)cccc2c1] 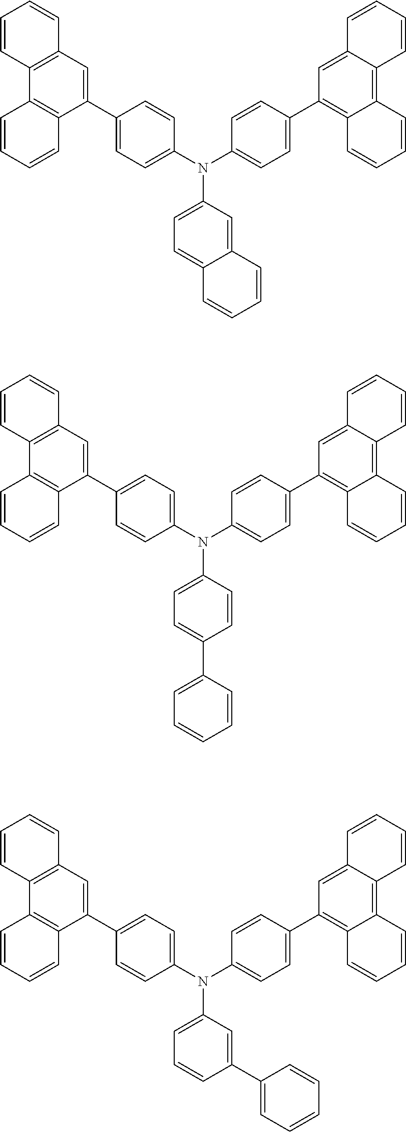 [SMILES: c1ccc2cc(N(c3ccc(-c4cc5ccccc5c5ccccc45)cc3)c3ccc(-c4cc5ccccc5c5ccccc45)cc3)ccc2c1 ; c1ccc(-c2ccc(N(c3ccc(-c4cc5ccccc5c5ccccc45)cc3)c3ccc(-c4cc5ccccc5c5ccccc45)cc3)cc2)cc1 ; c1ccc(-c2cccc(N(c3ccc(-c4cc5ccccc5c5ccccc45)cc3)c3ccc(-c4cc5ccccc5c5ccccc45)cc3)c2)cc1] 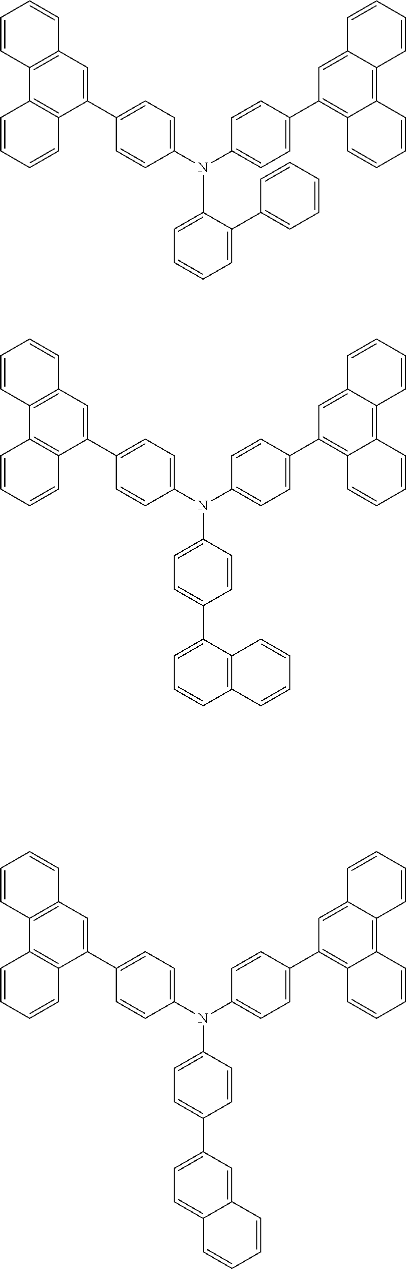 [SMILES: c1ccc(-c2ccccc2N(c2ccc(-c3cc4ccccc4c4ccccc34)cc2)c2ccc(-c3cc4ccccc4c4ccccc34)cc2)cc1 ; c1ccc2c(-c3ccc(N(c4ccc(-c5cc6ccccc6c6ccccc56)cc4)c4ccc(-c5cc6ccccc6c6ccccc56)cc4)cc3)cccc2c1 ; c1ccc2cc(-c3ccc(N(c4ccc(-c5cc6ccccc6c6ccccc56)cc4)c4ccc(-c5cc6ccccc6c6ccccc56)cc4)cc3)ccc2c1] 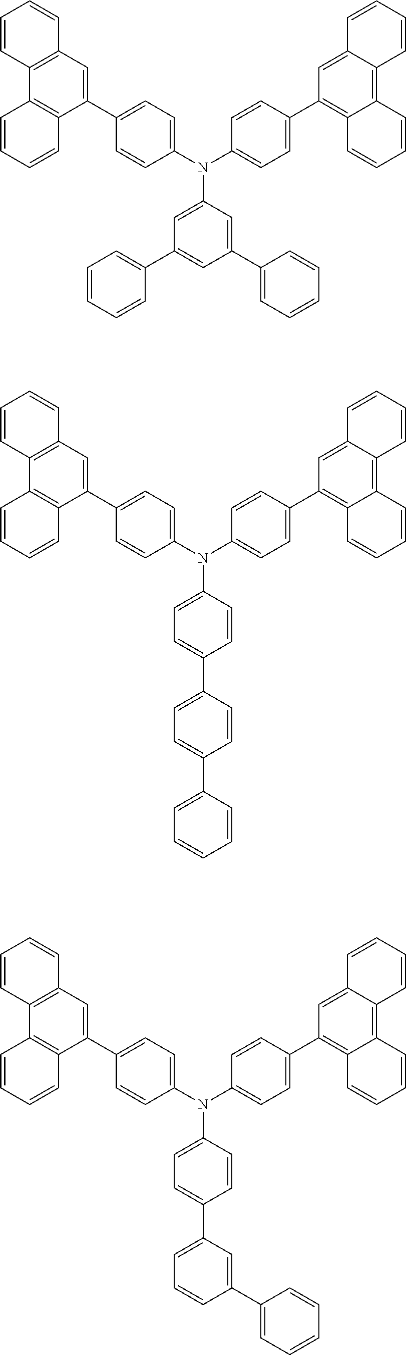 [SMILES: c1ccc(-c2cc(-c3ccccc3)cc(N(c3ccc(-c4cc5ccccc5c5ccccc45)cc3)c3ccc(-c4cc5ccccc5c5ccccc45)cc3)c2)cc1 ; c1ccc(-c2ccc(-c3ccc(N(c4ccc(-c5cc6ccccc6c6ccccc56)cc4)c4ccc(-c5cc6ccccc6c6ccccc56)cc4)cc3)cc2)cc1 ; c1ccc(-c2cccc(-c3ccc(N(c4ccc(-c5cc6ccccc6c6ccccc56)cc4)c4ccc(-c5cc6ccccc6c6ccccc56)cc4)cc3)c2)cc1] 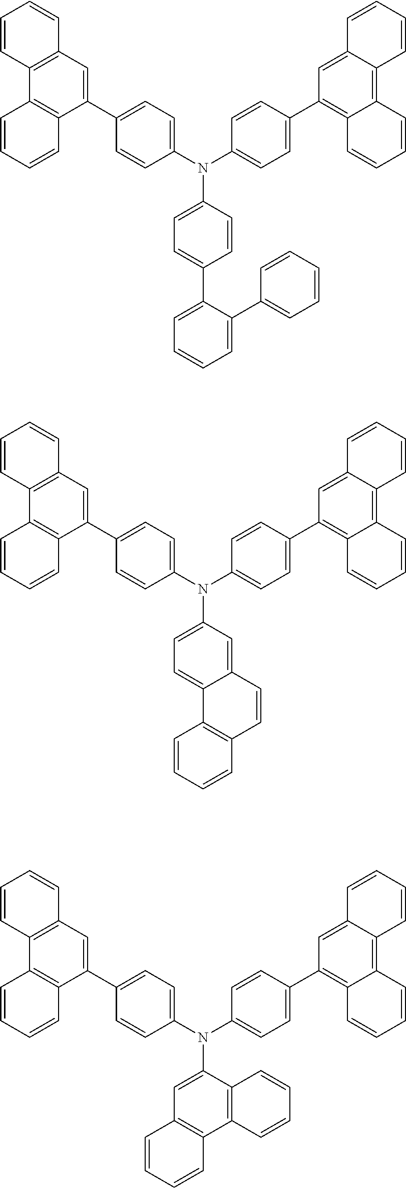 [SMILES: c1ccc(-c2ccccc2-c2ccc(N(c3ccc(-c4cc5ccccc5c5ccccc45)cc3)c3ccc(-c4cc5ccccc5c5ccccc45)cc3)cc2)cc1 ; c1ccc2c(c1)ccc1cc(N(c3ccc(-c4cc5ccccc5c5ccccc45)cc3)c3ccc(-c4cc5ccccc5c5ccccc45)cc3)ccc12 ; c1ccc2c(c1)cc(-c1ccc(N(c3ccc(-c4cc5ccccc5c5ccccc45)cc3)c3cc4ccccc4c4ccccc34)cc1)c1ccccc12] 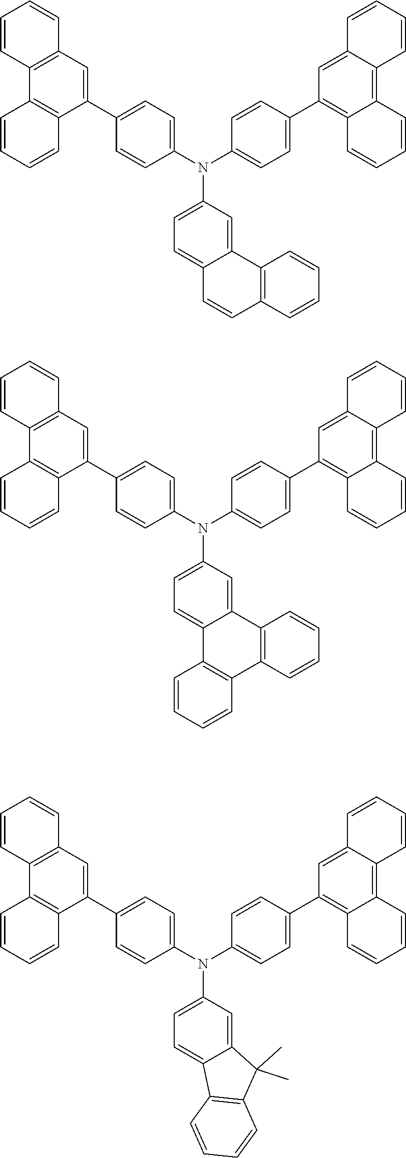 [SMILES: c1ccc2c(c1)ccc1ccc(N(c3ccc(-c4cc5ccccc5c5ccccc45)cc3)c3ccc(-c4cc5ccccc5c5ccccc45)cc3)cc12 ; c1ccc2c(c1)cc(-c1ccc(N(c3ccc(-c4cc5ccccc5c5ccccc45)cc3)c3ccc4c5ccccc5c5ccccc5c4c3)cc1)c1ccccc12 ; CC1(C)c2ccccc2-c2ccc(N(c3ccc(-c4cc5ccccc5c5ccccc45)cc3)c3ccc(-c4cc5ccccc5c5ccccc45)cc3)cc21] 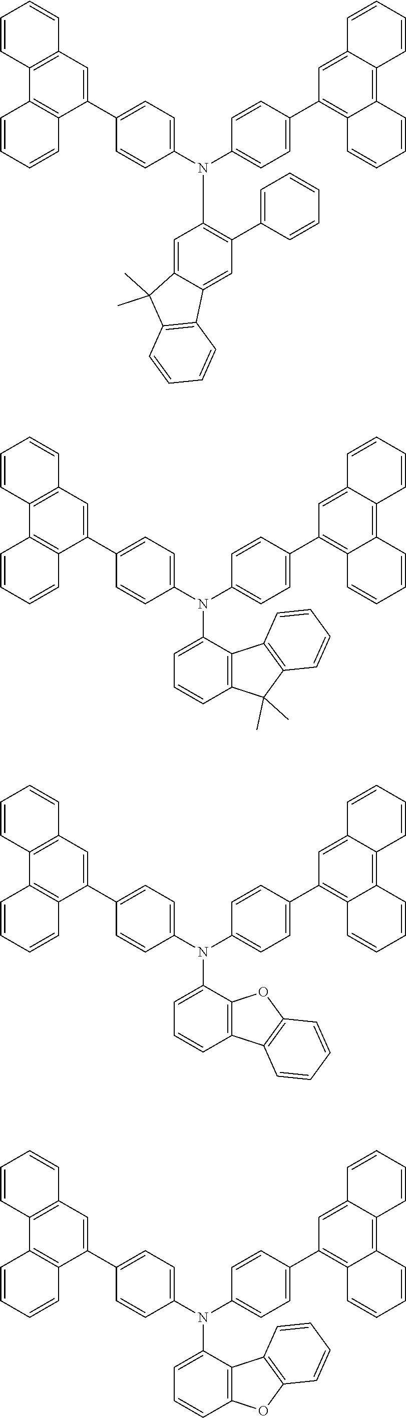 [SMILES: CC1(C)c2ccccc2-c2cc(-c3ccccc3)c(N(c3ccc(-c4cc5ccccc5c5ccccc45)cc3)c3ccc(-c4cc5ccccc5c5ccccc45)cc3)cc21 ; CC1(C)c2ccccc2-c2c(N(c3ccc(-c4cc5ccccc5c5ccccc45)cc3)c3ccc(-c4cc5ccccc5c5ccccc45)cc3)cccc21 ; c1ccc2c(c1)cc(-c1ccc(N(c3ccc(-c4cc5ccccc5c5ccccc45)cc3)c3cccc4c3oc3ccccc34)cc1)c1ccccc12 ; c1ccc2c(c1)cc(-c1ccc(N(c3ccc(-c4cc5ccccc5c5ccccc45)cc3)c3cccc4oc5ccccc5c34)cc1)c1ccccc12] 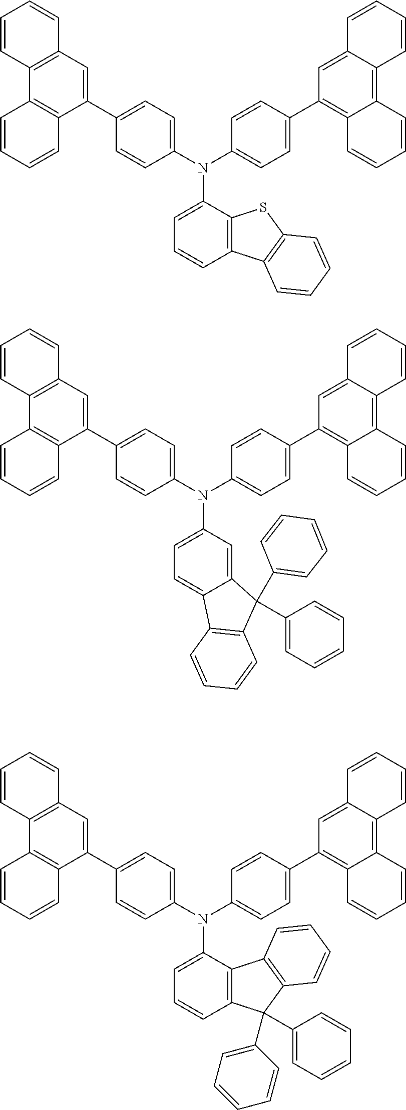 [SMILES: c1ccc2c(c1)cc(-c1ccc(N(c3ccc(-c4cc5ccccc5c5ccccc45)cc3)c3cccc4c3sc3ccccc34)cc1)c1ccccc12 ; c1ccc(C2(c3ccccc3)c3ccccc3-c3ccc(N(c4ccc(-c5cc6ccccc6c6ccccc56)cc4)c4ccc(-c5cc6ccccc6c6ccccc56)cc4)cc32)cc1 ; c1ccc(C2(c3ccccc3)c3ccccc3-c3c(N(c4ccc(-c5cc6ccccc6c6ccccc56)cc4)c4ccc(-c5cc6ccccc6c6ccccc56)cc4)cccc32)cc1] 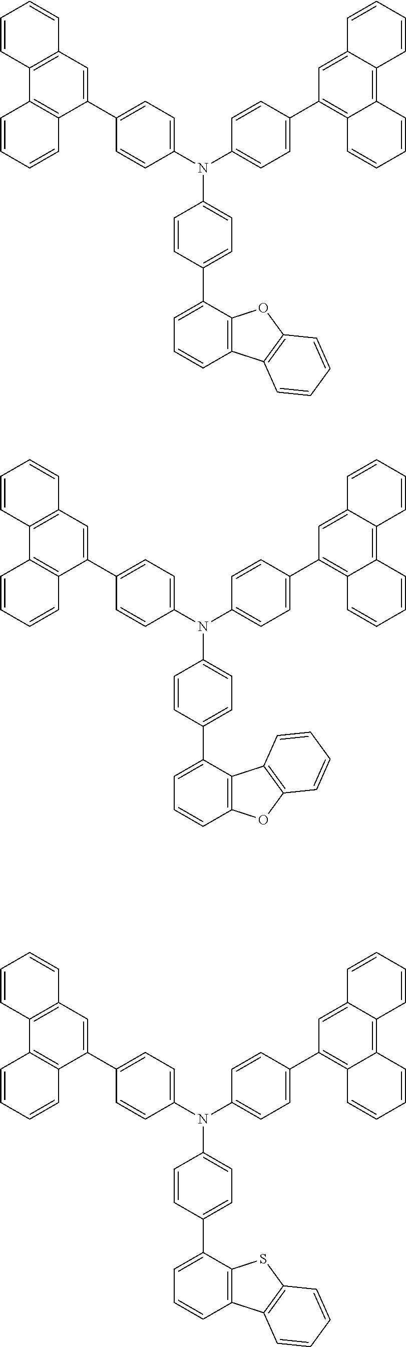 [SMILES: c1ccc2c(c1)cc(-c1ccc(N(c3ccc(-c4cc5ccccc5c5ccccc45)cc3)c3ccc(-c4cccc5c4oc4ccccc45)cc3)cc1)c1ccccc12 ; c1ccc2c(c1)cc(-c1ccc(N(c3ccc(-c4cc5ccccc5c5ccccc45)cc3)c3ccc(-c4cccc5oc6ccccc6c45)cc3)cc1)c1ccccc12 ; c1ccc2c(c1)cc(-c1ccc(N(c3ccc(-c4cc5ccccc5c5ccccc45)cc3)c3ccc(-c4cccc5c4sc4ccccc45)cc3)cc1)c1ccccc12] 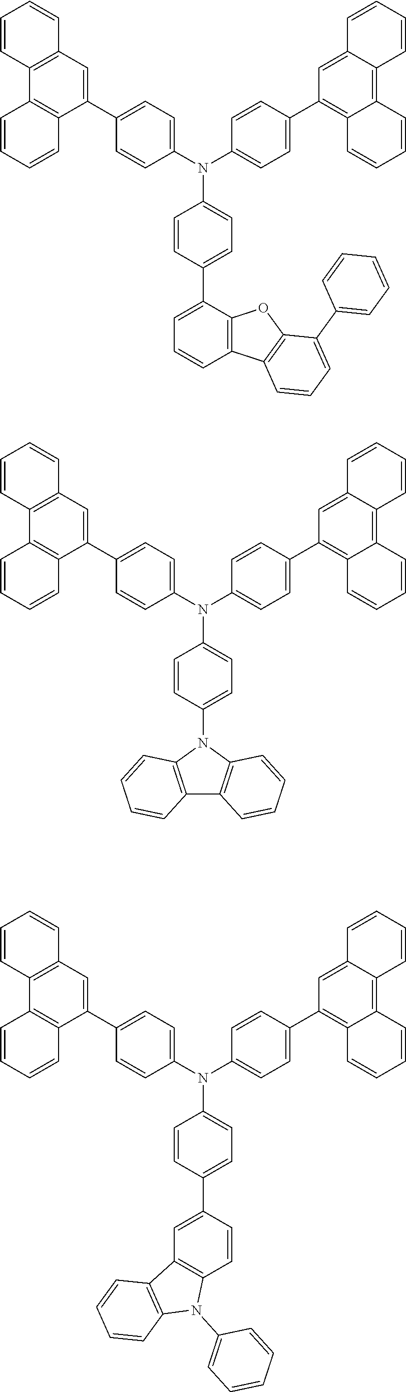 [SMILES: c1ccc(-c2cccc3c2oc2c(-c4ccc(N(c5ccc(-c6cc7ccccc7c7ccccc67)cc5)c5ccc(-c6cc7ccccc7c7ccccc67)cc5)cc4)cccc23)cc1 ; c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12 ; c1ccc(-n2c3ccccc3c3cc(-c4ccc(N(c5ccc(-c6cc7ccccc7c7ccccc67)cc5)c5ccc(-c6cc7ccccc7c7ccccc67)cc5)cc4)ccc32)cc1] 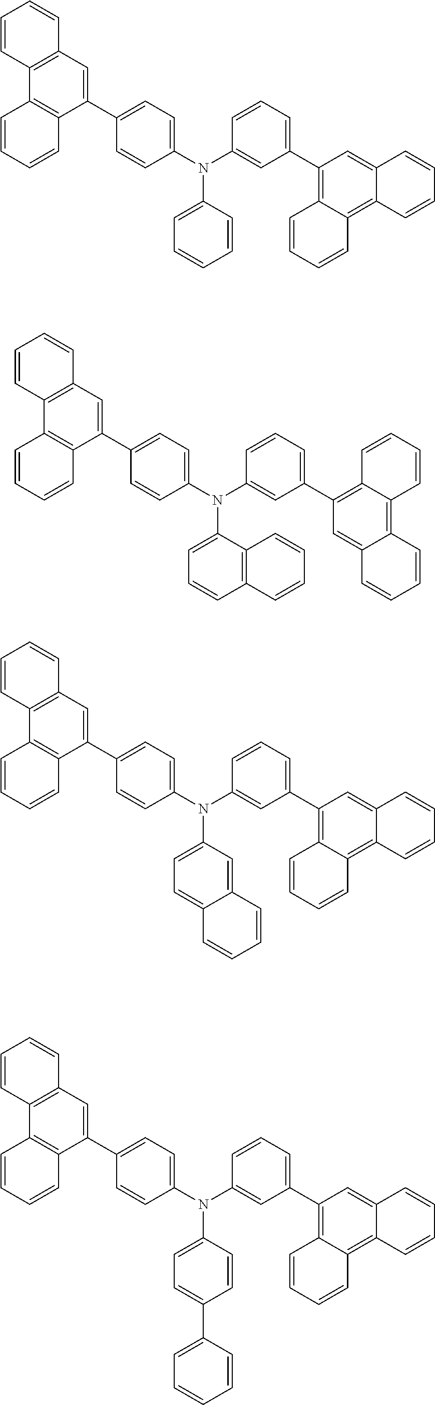 [SMILES: c1ccc(N(c2ccc(-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3cc4ccccc4c4ccccc34)cc2)c2cccc3ccccc23)c1 ; c1cc(-c2cc3ccccc3c3ccccc23)cc(N(c2ccc(-c3cc4ccccc4c4ccccc34)cc2)c2ccc3ccccc3c2)c1 ; c1ccc(-c2ccc(N(c3ccc(-c4cc5ccccc5c5ccccc45)cc3)c3cccc(-c4cc5ccccc5c5ccccc45)c3)cc2)cc1] 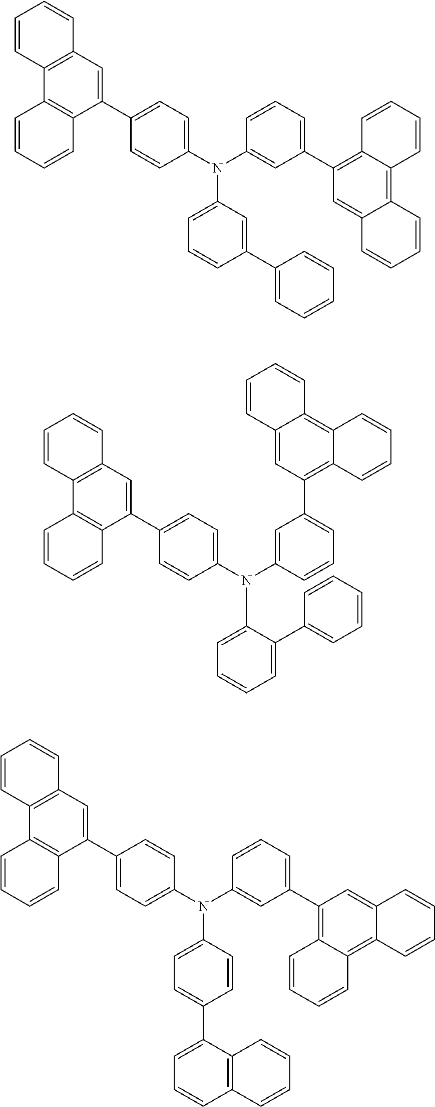 [SMILES: c1ccc(-c2cccc(N(c3ccc(-c4cc5ccccc5c5ccccc45)cc3)c3cccc(-c4cc5ccccc5c5ccccc45)c3)c2)cc1 ; c1ccc(-c2ccccc2N(c2ccc(-c3cc4ccccc4c4ccccc34)cc2)c2cccc(-c3cc4ccccc4c4ccccc34)c2)cc1 ; c1cc(-c2cc3ccccc3c3ccccc23)cc(N(c2ccc(-c3cccc4ccccc34)cc2)c2ccc(-c3cc4ccccc4c4ccccc34)cc2)c1] 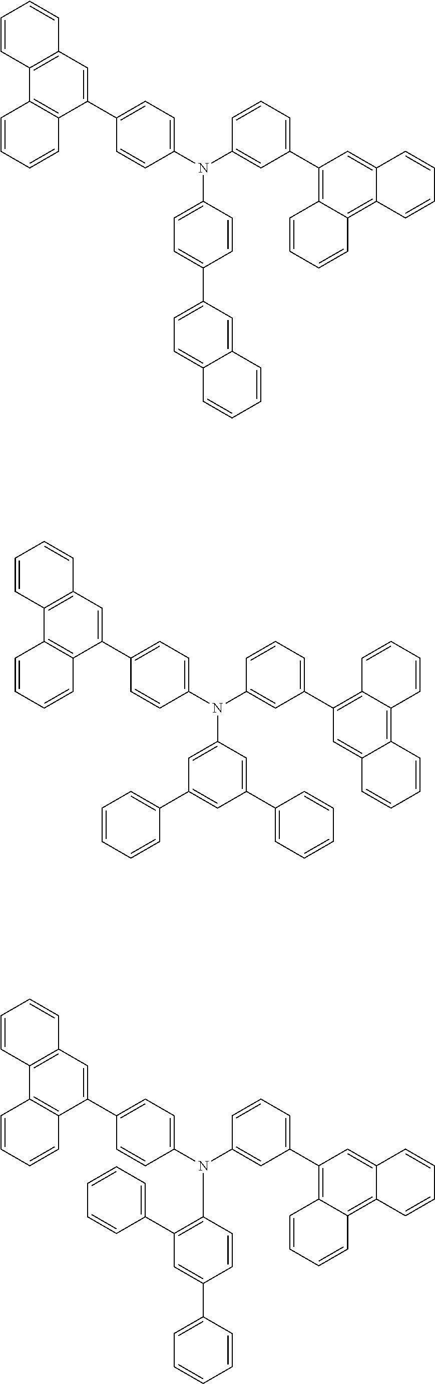 [SMILES: c1cc(-c2cc3ccccc3c3ccccc23)cc(N(c2ccc(-c3ccc4ccccc4c3)cc2)c2ccc(-c3cc4ccccc4c4ccccc34)cc2)c1 ; c1ccc(-c2cc(-c3ccccc3)cc(N(c3ccc(-c4cc5ccccc5c5ccccc45)cc3)c3cccc(-c4cc5ccccc5c5ccccc45)c3)c2)cc1 ; c1ccc(-c2ccc(N(c3ccc(-c4cc5ccccc5c5ccccc45)cc3)c3cccc(-c4cc5ccccc5c5ccccc45)c3)c(-c3ccccc3)c2)cc1] 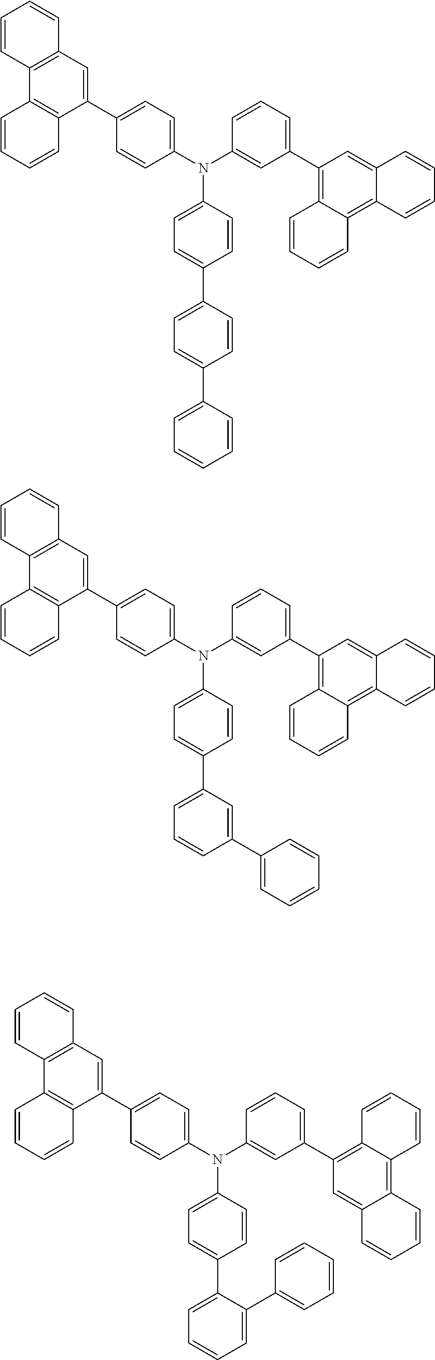 [SMILES: c1ccc(-c2ccc(-c3ccc(N(c4ccc(-c5cc6ccccc6c6ccccc56)cc4)c4cccc(-c5cc6ccccc6c6ccccc56)c4)cc3)cc2)cc1 ; c1ccc(-c2cccc(-c3ccc(N(c4ccc(-c5cc6ccccc6c6ccccc56)cc4)c4cccc(-c5cc6ccccc6c6ccccc56)c4)cc3)c2)cc1 ; c1ccc(-c2ccccc2-c2ccc(N(c3ccc(-c4cc5ccccc5c5ccccc45)cc3)c3cccc(-c4cc5ccccc5c5ccccc45)c3)cc2)cc1] 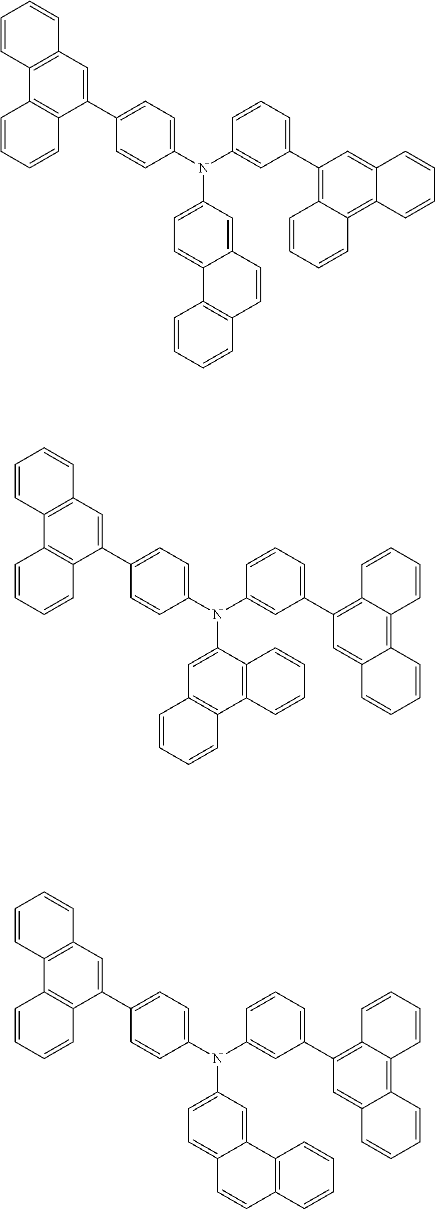 [SMILES: c1cc(-c2cc3ccccc3c3ccccc23)cc(N(c2ccc(-c3cc4ccccc4c4ccccc34)cc2)c2ccc3c(ccc4ccccc43)c2)c1 ; c1cc(-c2cc3ccccc3c3ccccc23)cc(N(c2ccc(-c3cc4ccccc4c4ccccc34)cc2)c2cc3ccccc3c3ccccc23)c1 ; c1cc(-c2cc3ccccc3c3ccccc23)cc(N(c2ccc(-c3cc4ccccc4c4ccccc34)cc2)c2ccc3ccc4ccccc4c3c2)c1] 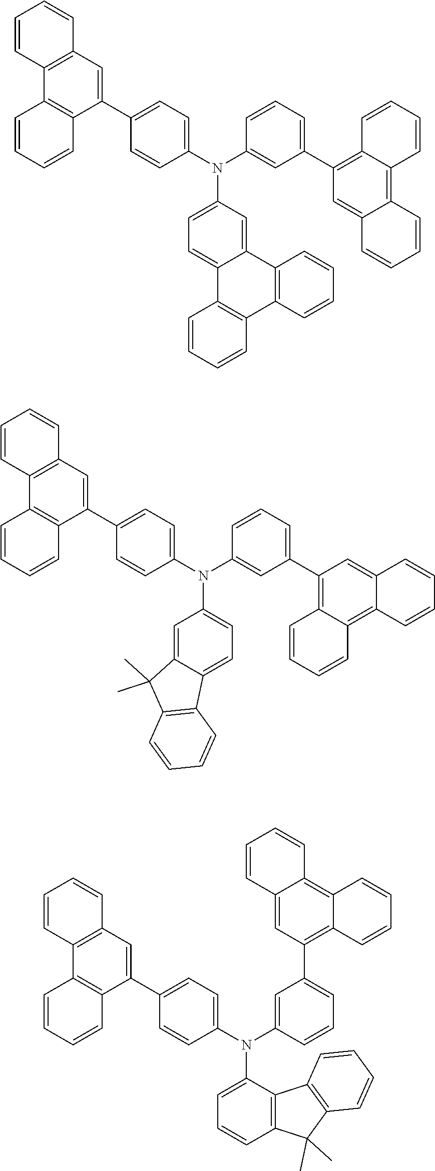 [SMILES: c1cc(-c2cc3ccccc3c3ccccc23)cc(N(c2ccc(-c3cc4ccccc4c4ccccc34)cc2)c2ccc3c4ccccc4c4ccccc4c3c2)c1 ; CC1(C)c2ccccc2-c2ccc(N(c3ccc(-c4cc5ccccc5c5ccccc45)cc3)c3cccc(-c4cc5ccccc5c5ccccc45)c3)cc21 ; CC1(C)c2ccccc2-c2c(N(c3ccc(-c4cc5ccccc5c5ccccc45)cc3)c3cccc(-c4cc5ccccc5c5ccccc45)c3)cccc21] 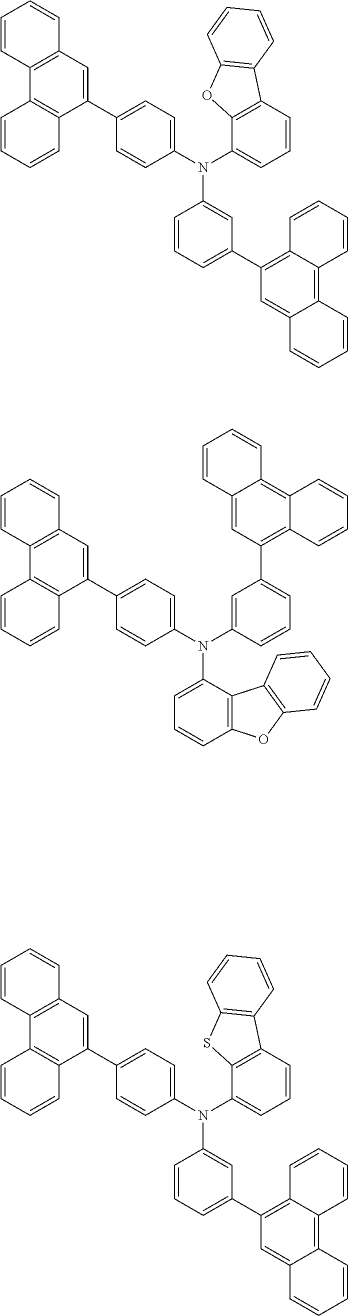 [SMILES: c1cc(-c2cc3ccccc3c3ccccc23)cc(N(c2ccc(-c3cc4ccccc4c4ccccc34)cc2)c2cccc3c2oc2ccccc23)c1 ; c1cc(-c2cc3ccccc3c3ccccc23)cc(N(c2ccc(-c3cc4ccccc4c4ccccc34)cc2)c2cccc3oc4ccccc4c23)c1 ; c1cc(-c2cc3ccccc3c3ccccc23)cc(N(c2ccc(-c3cc4ccccc4c4ccccc34)cc2)c2cccc3c2sc2ccccc23)c1] 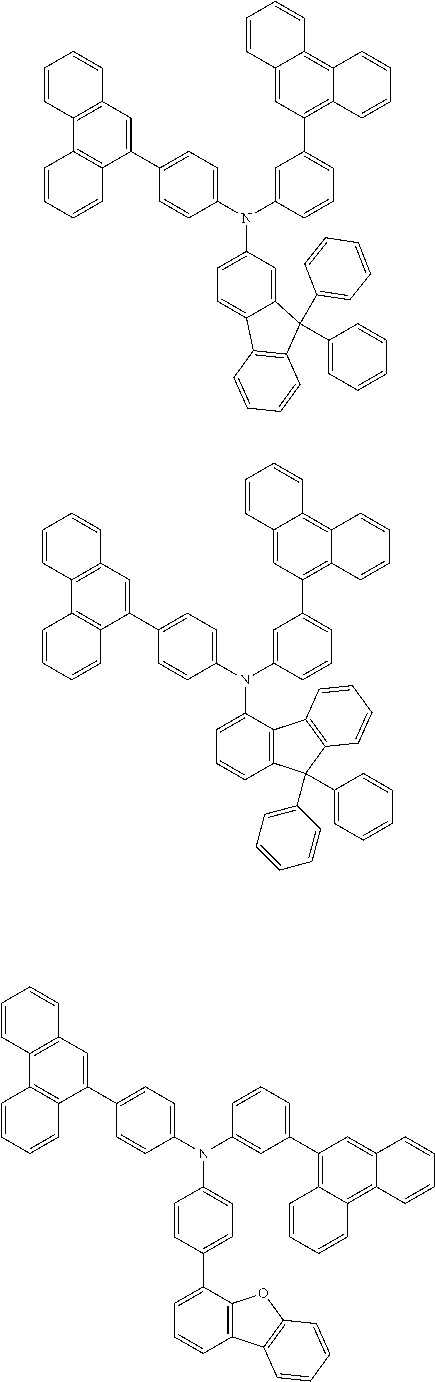 [SMILES: c1ccc(C2(c3ccccc3)c3ccccc3-c3ccc(N(c4ccc(-c5cc6ccccc6c6ccccc56)cc4)c4cccc(-c5cc6ccccc6c6ccccc56)c4)cc32)cc1 ; c1ccc(C2(c3ccccc3)c3ccccc3-c3c(N(c4ccc(-c5cc6ccccc6c6ccccc56)cc4)c4cccc(-c5cc6ccccc6c6ccccc56)c4)cccc32)cc1 ; c1cc(-c2cc3ccccc3c3ccccc23)cc(N(c2ccc(-c3cc4ccccc4c4ccccc34)cc2)c2ccc(-c3cccc4c3oc3ccccc34)cc2)c1] 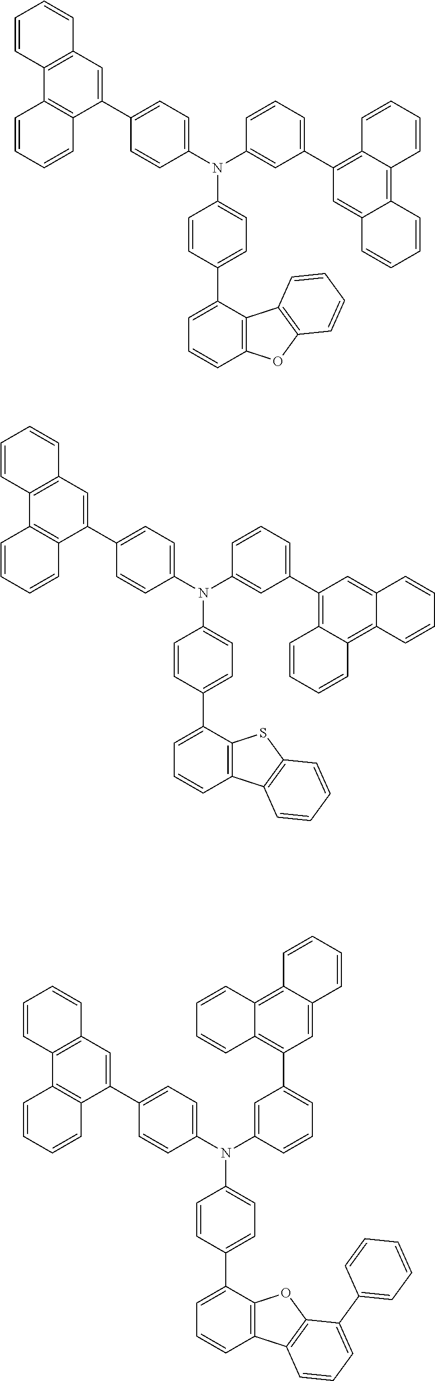 [SMILES: c1cc(-c2cc3ccccc3c3ccccc23)cc(N(c2ccc(-c3cc4ccccc4c4ccccc34)cc2)c2ccc(-c3cccc4oc5ccccc5c34)cc2)c1 ; c1cc(-c2cc3ccccc3c3ccccc23)cc(N(c2ccc(-c3cc4ccccc4c4ccccc34)cc2)c2ccc(-c3cccc4c3sc3ccccc34)cc2)c1 ; c1ccc(-c2cccc3c2oc2c(-c4ccc(N(c5ccc(-c6cc7ccccc7c7ccccc67)cc5)c5cccc(-c6cc7ccccc7c7ccccc67)c5)cc4)cccc23)cc1] 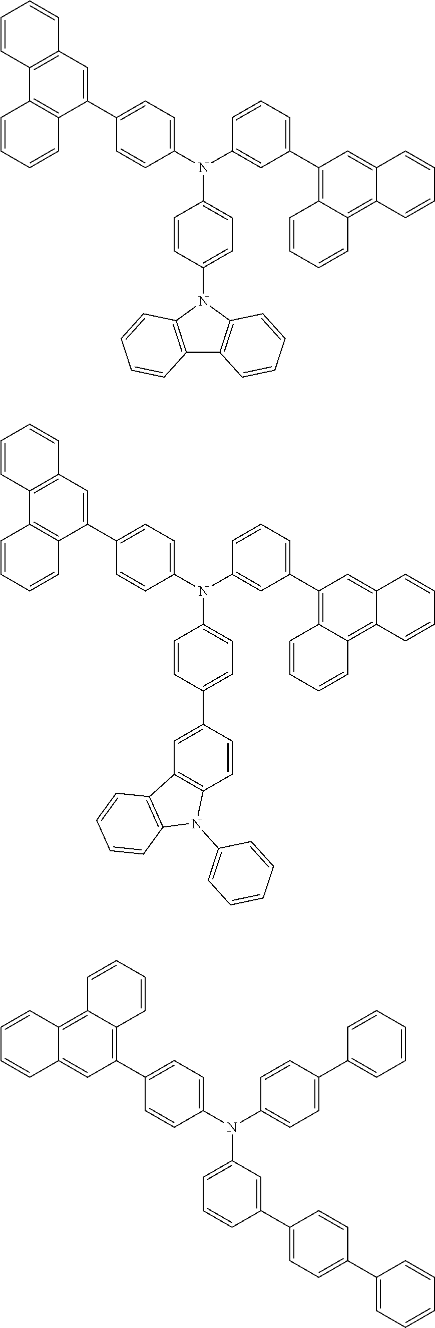 [SMILES: c1cc(-c2cc3ccccc3c3ccccc23)cc(N(c2ccc(-c3cc4ccccc4c4ccccc34)cc2)c2ccc(-n3c4ccccc4c4ccccc43)cc2)c1 ; c1ccc(-n2c3ccccc3c3cc(-c4ccc(N(c5ccc(-c6cc7ccccc7c7ccccc67)cc5)c5cccc(-c6cc7ccccc7c7ccccc67)c5)cc4)ccc32)cc1 ; c1ccc(-c2ccc(-c3cccc(N(c4ccc(-c5ccccc5)cc4)c4ccc(-c5cc6ccccc6c6ccccc56)cc4)c3)cc2)cc1] 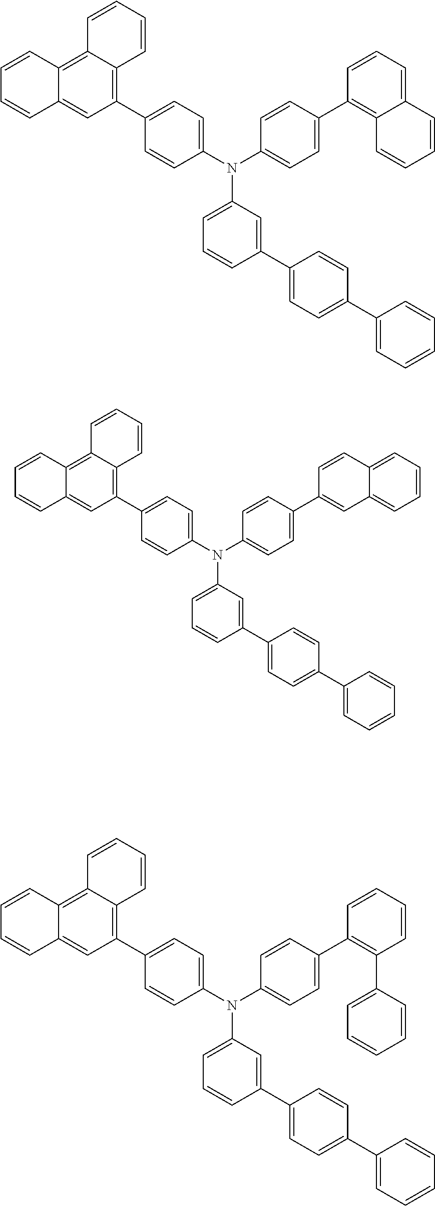 [SMILES: c1ccc(-c2ccc(-c3cccc(N(c4ccc(-c5cccc6ccccc56)cc4)c4ccc(-c5cc6ccccc6c6ccccc56)cc4)c3)cc2)cc1 ; c1ccc(-c2ccc(-c3cccc(N(c4ccc(-c5ccc6ccccc6c5)cc4)c4ccc(-c5cc6ccccc6c6ccccc56)cc4)c3)cc2)cc1 ; c1ccc(-c2ccc(-c3cccc(N(c4ccc(-c5ccccc5-c5ccccc5)cc4)c4ccc(-c5cc6ccccc6c6ccccc56)cc4)c3)cc2)cc1] 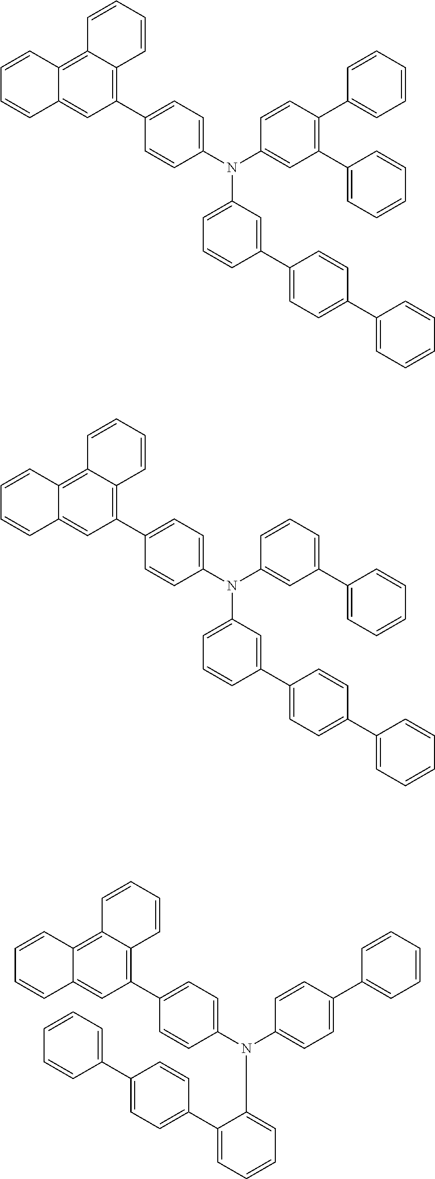 [SMILES: c1ccc(-c2ccc(-c3cccc(N(c4ccc(-c5cc6ccccc6c6ccccc56)cc4)c4ccc(-c5ccccc5)c(-c5ccccc5)c4)c3)cc2)cc1 ; c1ccc(-c2ccc(-c3cccc(N(c4ccc(-c5cc6ccccc6c6ccccc56)cc4)c4cccc(-c5ccccc5)c4)c3)cc2)cc1 ; c1ccc(-c2ccc(-c3ccccc3N(c3ccc(-c4ccccc4)cc3)c3ccc(-c4cc5ccccc5c5ccccc45)cc3)cc2)cc1] 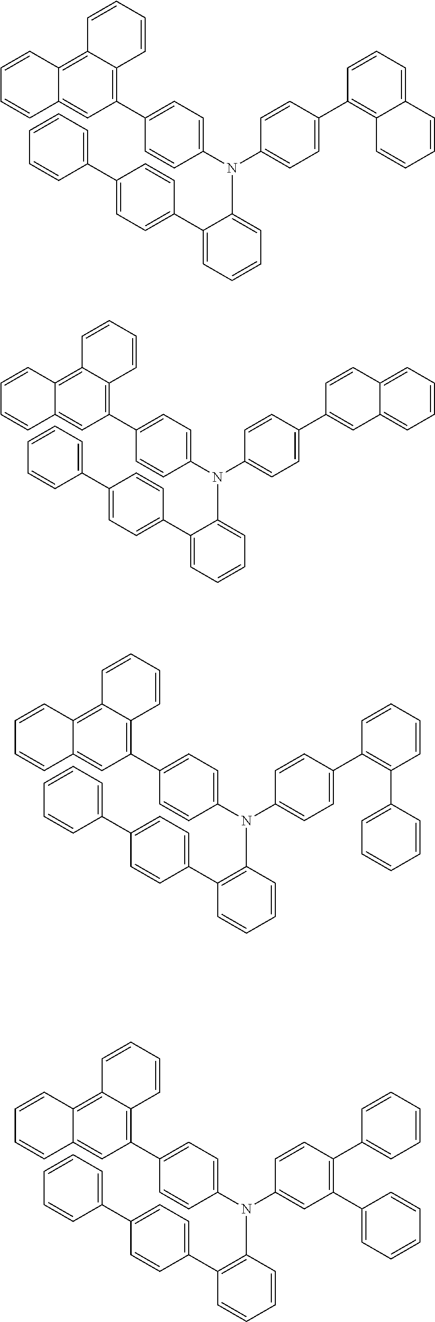 [SMILES: c1ccc(-c2ccc(-c3ccccc3N(c3ccc(-c4cccc5ccccc45)cc3)c3ccc(-c4cc5ccccc5c5ccccc45)cc3)cc2)cc1 ; c1ccc(-c2ccc(-c3ccccc3N(c3ccc(-c4ccc5ccccc5c4)cc3)c3ccc(-c4cc5ccccc5c5ccccc45)cc3)cc2)cc1 ; c1ccc(-c2ccc(-c3ccccc3N(c3ccc(-c4ccccc4-c4ccccc4)cc3)c3ccc(-c4cc5ccccc5c5ccccc45)cc3)cc2)cc1 ; c1ccc(-c2ccc(-c3ccccc3N(c3ccc(-c4cc5ccccc5c5ccccc45)cc3)c3ccc(-c4ccccc4)c(-c4ccccc4)c3)cc2)cc1] 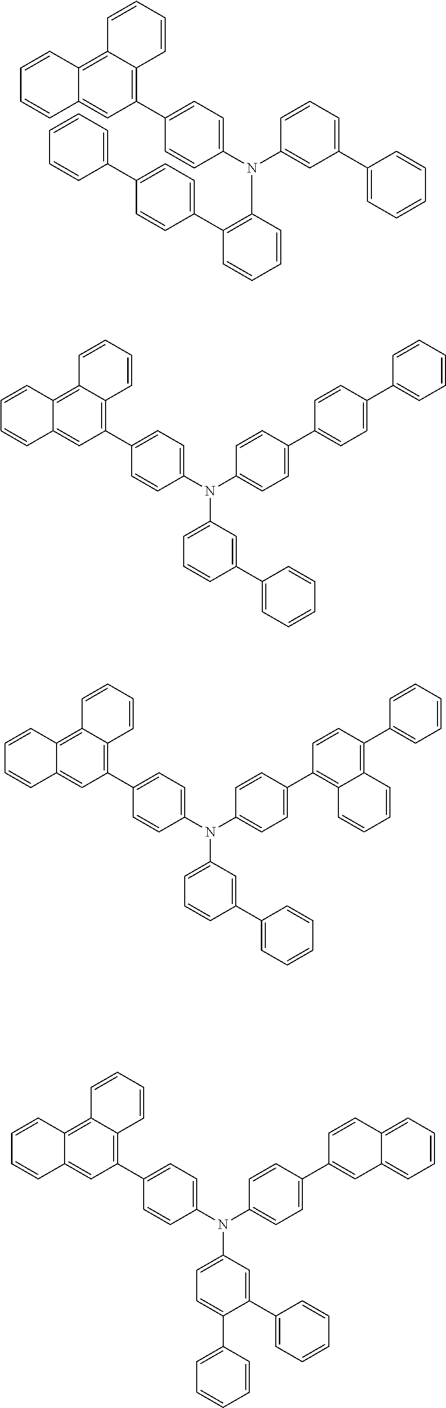 [SMILES: c1ccc(-c2ccc(-c3ccccc3N(c3ccc(-c4cc5ccccc5c5ccccc45)cc3)c3cccc(-c4ccccc4)c3)cc2)cc1 ; c1ccc(-c2ccc(-c3ccc(N(c4ccc(-c5cc6ccccc6c6ccccc56)cc4)c4cccc(-c5ccccc5)c4)cc3)cc2)cc1 ; c1ccc(-c2cccc(N(c3ccc(-c4ccc(-c5ccccc5)c5ccccc45)cc3)c3ccc(-c4cc5ccccc5c5ccccc45)cc3)c2)cc1 ; c1ccc(-c2ccc(N(c3ccc(-c4ccc5ccccc5c4)cc3)c3ccc(-c4cc5ccccc5c5ccccc45)cc3)cc2-c2ccccc2)cc1] 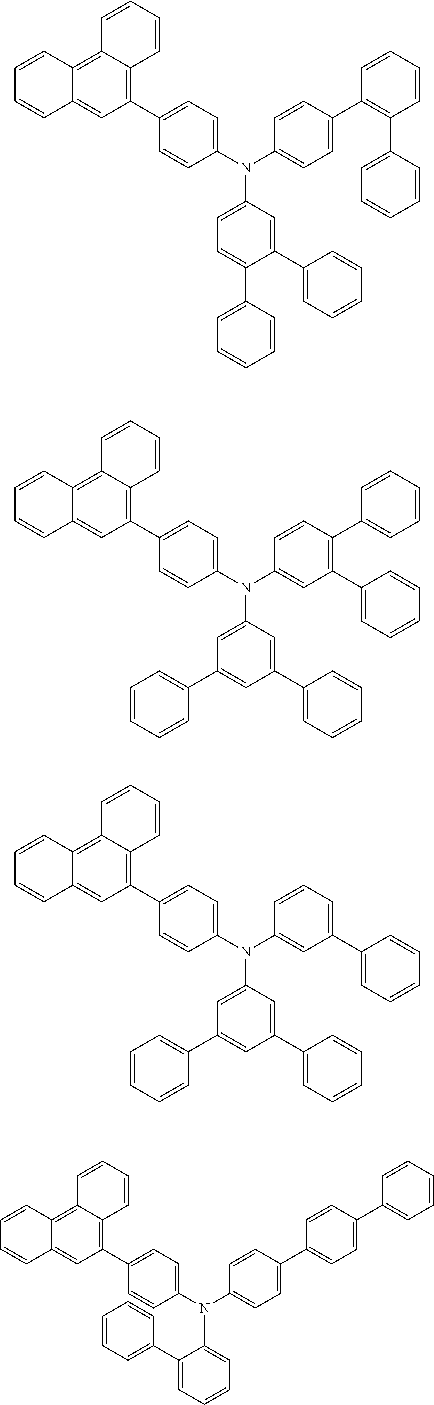 [SMILES: c1ccc(-c2ccccc2-c2ccc(N(c3ccc(-c4cc5ccccc5c5ccccc45)cc3)c3ccc(-c4ccccc4)c(-c4ccccc4)c3)cc2)cc1 ; c1ccc(-c2cc(-c3ccccc3)cc(N(c3ccc(-c4cc5ccccc5c5ccccc45)cc3)c3ccc(-c4ccccc4)c(-c4ccccc4)c3)c2)cc1 ; c1ccc(-c2cccc(N(c3ccc(-c4cc5ccccc5c5ccccc45)cc3)c3cc(-c4ccccc4)cc(-c4ccccc4)c3)c2)cc1 ; c1ccc(-c2ccc(-c3ccc(N(c4ccc(-c5cc6ccccc6c6ccccc56)cc4)c4ccccc4-c4ccccc4)cc3)cc2)cc1] 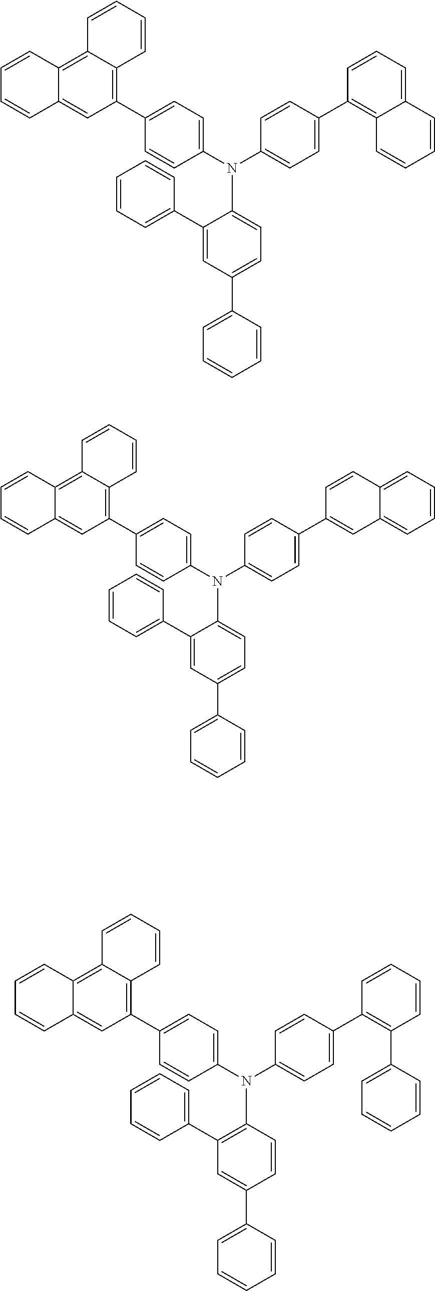 [SMILES: c1ccc(-c2ccc(N(c3ccc(-c4cccc5ccccc45)cc3)c3ccc(-c4cc5ccccc5c5ccccc45)cc3)c(-c3ccccc3)c2)cc1 ; c1ccc(-c2ccc(N(c3ccc(-c4ccc5ccccc5c4)cc3)c3ccc(-c4cc5ccccc5c5ccccc45)cc3)c(-c3ccccc3)c2)cc1 ; c1ccc(-c2ccc(N(c3ccc(-c4ccccc4-c4ccccc4)cc3)c3ccc(-c4cc5ccccc5c5ccccc45)cc3)c(-c3ccccc3)c2)cc1] 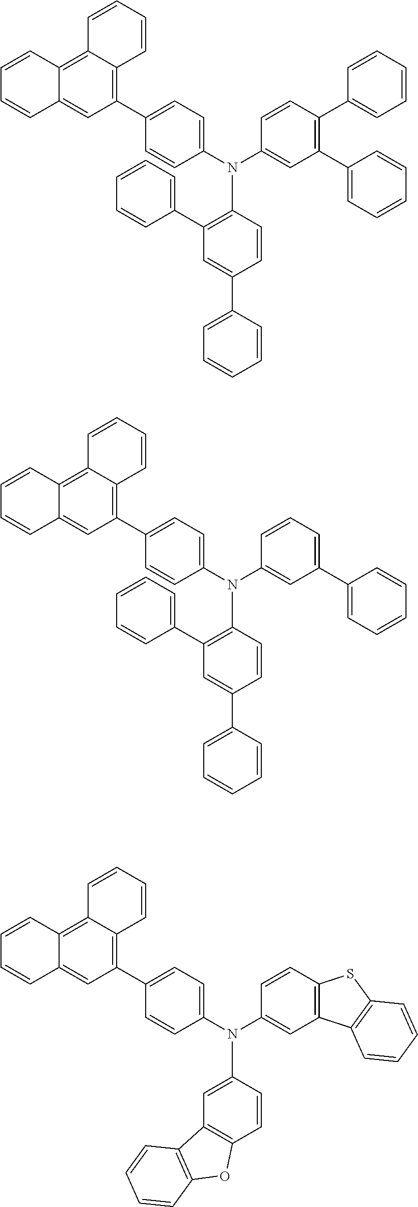 [SMILES: c1ccc(-c2ccc(N(c3ccc(-c4cc5ccccc5c5ccccc45)cc3)c3ccc(-c4ccccc4)c(-c4ccccc4)c3)c(-c3ccccc3)c2)cc1 ; c1ccc(-c2cccc(N(c3ccc(-c4cc5ccccc5c5ccccc45)cc3)c3ccc(-c4ccccc4)cc3-c3ccccc3)c2)cc1 ; c1ccc2c(c1)cc(-c1ccc(N(c3ccc4oc5ccccc5c4c3)c3ccc4sc5ccccc5c4c3)cc1)c1ccccc12] 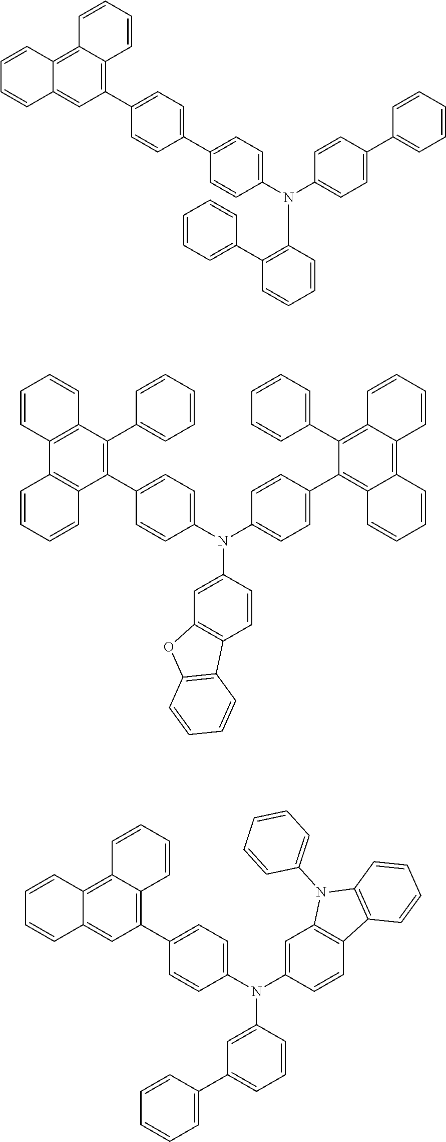 [SMILES: c1ccc(-c2ccc(N(c3ccc(-c4ccc(-c5cc6ccccc6c6ccccc56)cc4)cc3)c3ccccc3-c3ccccc3)cc2)cc1 ; c1ccc(-c2c(-c3ccc(N(c4ccc(-c5c(-c6ccccc6)c6ccccc6c6ccccc56)cc4)c4ccc5c(c4)oc4ccccc45)cc3)c3ccccc3c3ccccc23)cc1 ; c1ccc(-c2cccc(N(c3ccc(-c4cc5ccccc5c5ccccc45)cc3)c3ccc4c5ccccc5n(-c5ccccc5)c4c3)c2)cc1] 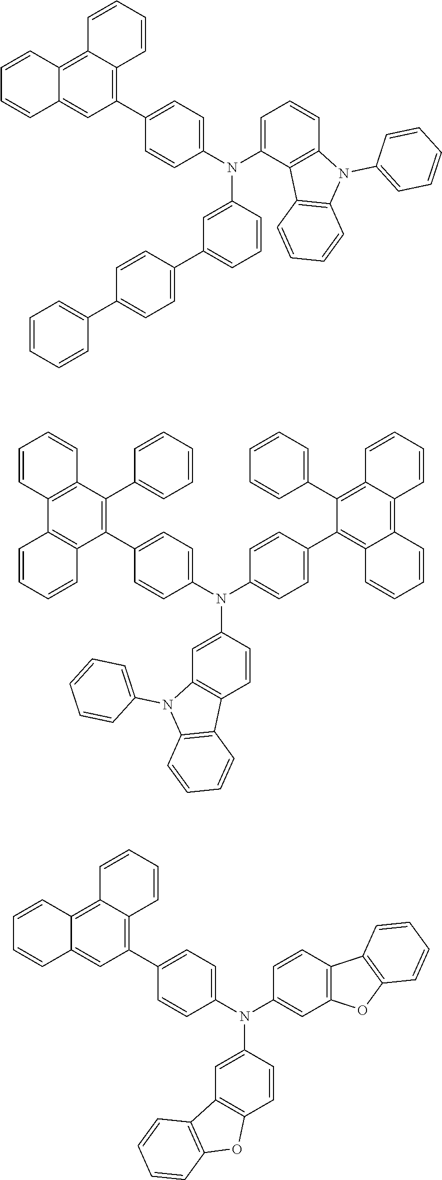 [SMILES: c1ccc(-c2ccc(-c3cccc(N(c4ccc(-c5cc6ccccc6c6ccccc56)cc4)c4cccc5c4c4ccccc4n5-c4ccccc4)c3)cc2)cc1 ; c1ccc(-c2c(-c3ccc(N(c4ccc(-c5c(-c6ccccc6)c6ccccc6c6ccccc56)cc4)c4ccc5c6ccccc6n(-c6ccccc6)c5c4)cc3)c3ccccc3c3ccccc23)cc1 ; c1ccc2c(c1)cc(-c1ccc(N(c3ccc4c(c3)oc3ccccc34)c3ccc4oc5ccccc5c4c3)cc1)c1ccccc12] 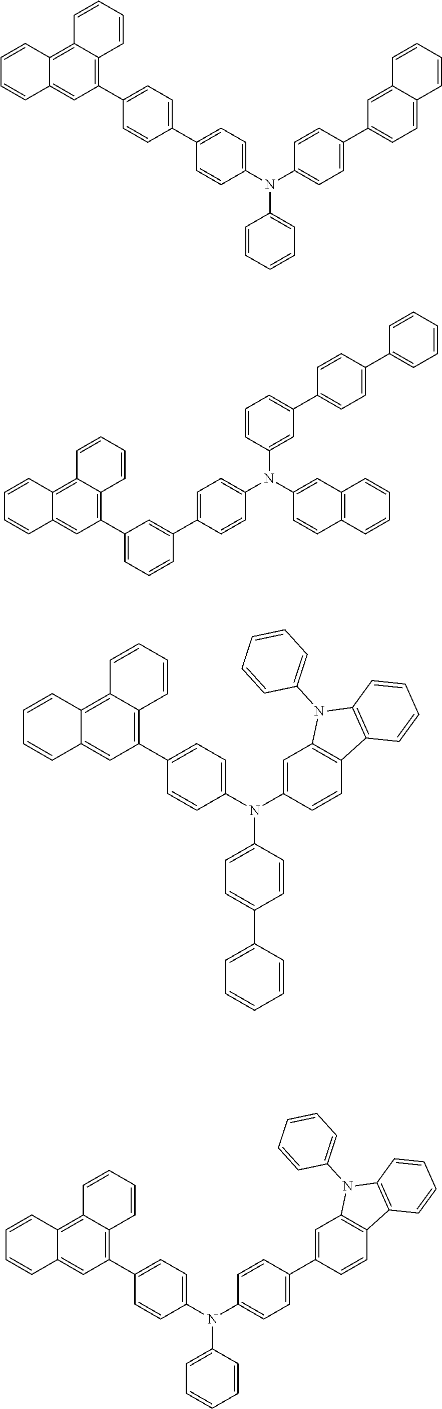 [SMILES: c1ccc(N(c2ccc(-c3ccc(-c4cc5ccccc5c5ccccc45)cc3)cc2)c2ccc(-c3ccc4ccccc4c3)cc2)cc1 ; c1ccc(-c2ccc(-c3cccc(N(c4ccc(-c5cccc(-c6cc7ccccc7c7ccccc67)c5)cc4)c4ccc5ccccc5c4)c3)cc2)cc1 ; c1ccc(-c2ccc(N(c3ccc(-c4cc5ccccc5c5ccccc45)cc3)c3ccc4c5ccccc5n(-c5ccccc5)c4c3)cc2)cc1 ; c1ccc(N(c2ccc(-c3ccc4c5ccccc5n(-c5ccccc5)c4c3)cc2)c2ccc(-c3cc4ccccc4c4ccccc34)cc2)cc1] 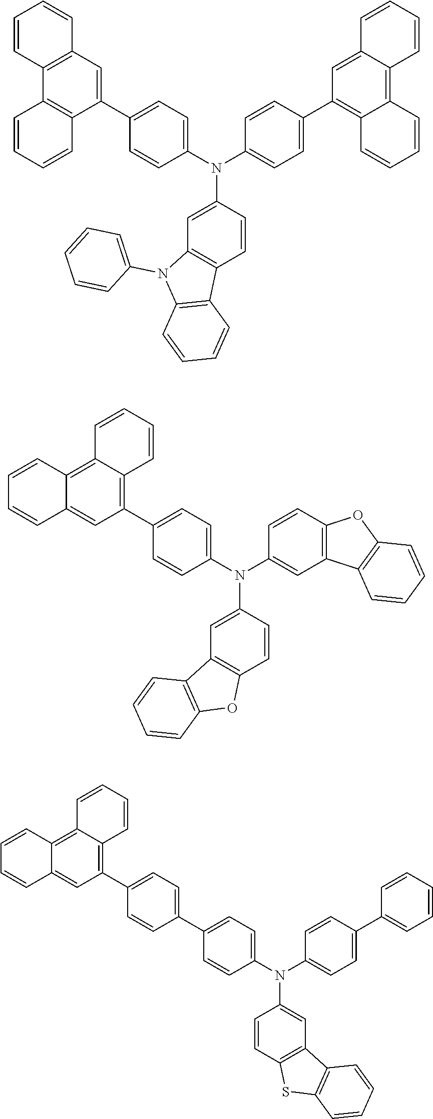 [SMILES: c1ccc(-n2c3ccccc3c3ccc(N(c4ccc(-c5cc6ccccc6c6ccccc56)cc4)c4ccc(-c5cc6ccccc6c6ccccc56)cc4)cc32)cc1 ; c1ccc2c(c1)cc(-c1ccc(N(c3ccc4oc5ccccc5c4c3)c3ccc4oc5ccccc5c4c3)cc1)c1ccccc12 ; c1ccc(-c2ccc(N(c3ccc(-c4ccc(-c5cc6ccccc6c6ccccc56)cc4)cc3)c3ccc4sc5ccccc5c4c3)cc2)cc1] 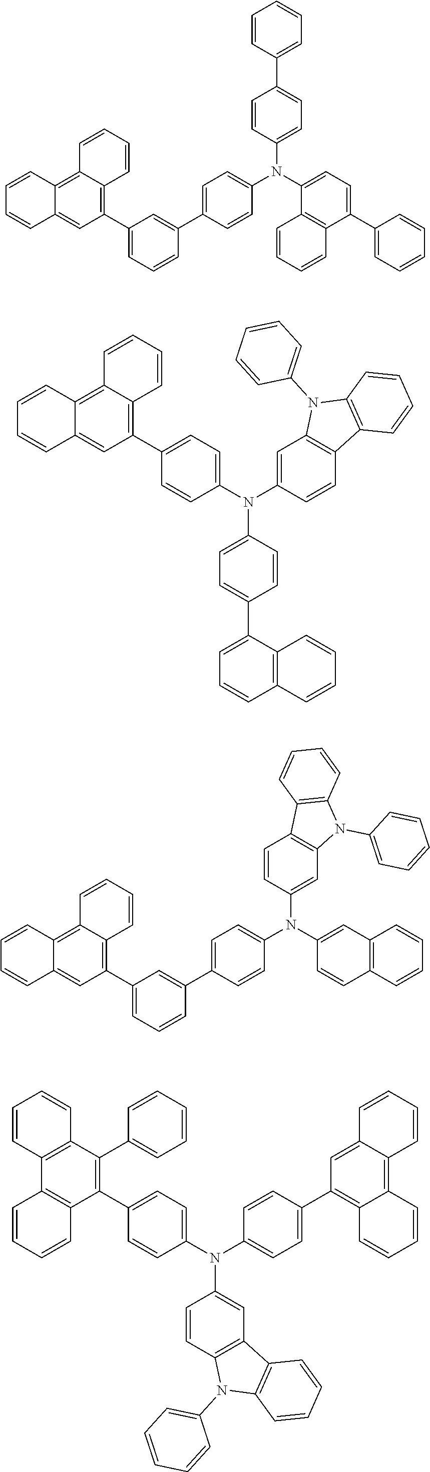 [SMILES: c1ccc(-c2ccc(N(c3ccc(-c4cccc(-c5cc6ccccc6c6ccccc56)c4)cc3)c3ccc(-c4ccccc4)c4ccccc34)cc2)cc1 ; c1ccc(-n2c3ccccc3c3ccc(N(c4ccc(-c5cccc6ccccc56)cc4)c4ccc(-c5cc6ccccc6c6ccccc56)cc4)cc32)cc1 ; c1ccc(-n2c3ccccc3c3ccc(N(c4ccc(-c5cccc(-c6cc7ccccc7c7ccccc67)c5)cc4)c4ccc5ccccc5c4)cc32)cc1 ; c1ccc(-c2c(-c3ccc(N(c4ccc(-c5cc6ccccc6c6ccccc56)cc4)c4ccc5c(c4)c4ccccc4n5-c4ccccc4)cc3)c3ccccc3c3ccccc23)cc1] 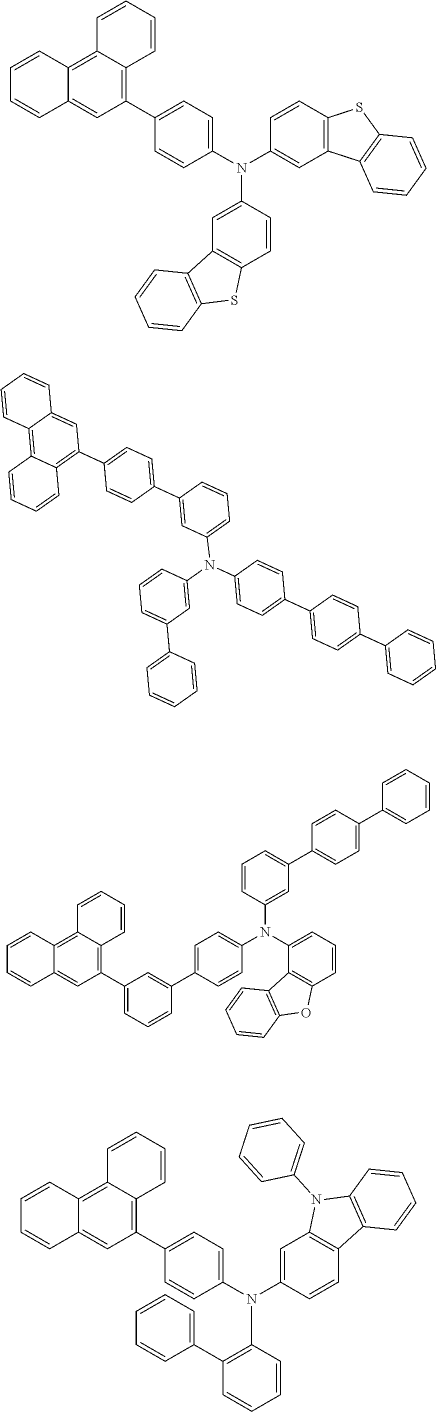 [SMILES: c1ccc2c(c1)cc(-c1ccc(N(c3ccc4sc5ccccc5c4c3)c3ccc4sc5ccccc5c4c3)cc1)c1ccccc12 ; c1ccc(-c2ccc(-c3ccc(N(c4cccc(-c5ccccc5)c4)c4cccc(-c5ccc(-c6cc7ccccc7c7ccccc67)cc5)c4)cc3)cc2)cc1 ; c1ccc(-c2ccc(-c3cccc(N(c4ccc(-c5cccc(-c6cc7ccccc7c7ccccc67)c5)cc4)c4cccc5oc6ccccc6c45)c3)cc2)cc1 ; c1ccc(-c2ccccc2N(c2ccc(-c3cc4ccccc4c4ccccc34)cc2)c2ccc3c4ccccc4n(-c4ccccc4)c3c2)cc1] 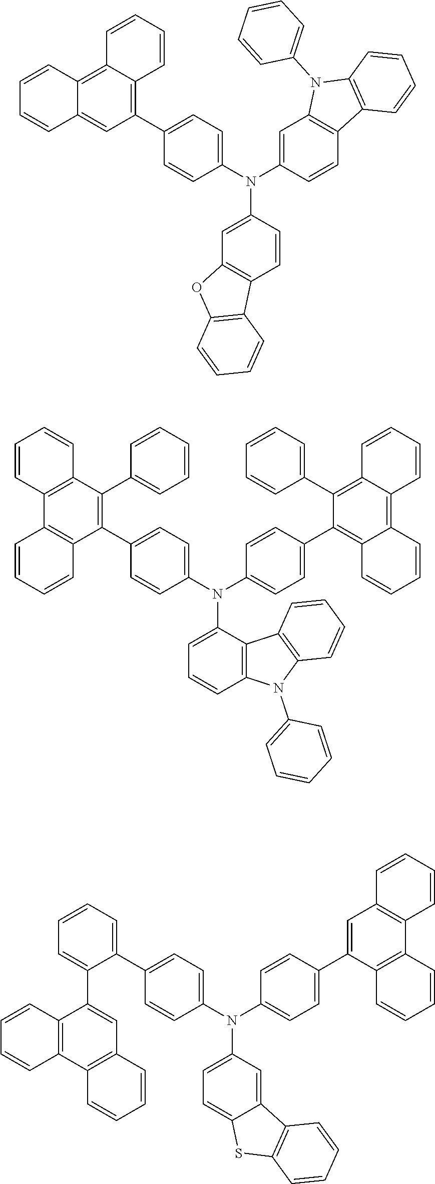 [SMILES: c1ccc(-n2c3ccccc3c3ccc(N(c4ccc(-c5cc6ccccc6c6ccccc56)cc4)c4ccc5c(c4)oc4ccccc45)cc32)cc1 ; c1ccc(-c2c(-c3ccc(N(c4ccc(-c5c(-c6ccccc6)c6ccccc6c6ccccc56)cc4)c4cccc5c4c4ccccc4n5-c4ccccc4)cc3)c3ccccc3c3ccccc23)cc1 ; c1ccc(-c2cc3ccccc3c3ccccc23)c(-c2ccc(N(c3ccc(-c4cc5ccccc5c5ccccc45)cc3)c3ccc4sc5ccccc5c4c3)cc2)c1] 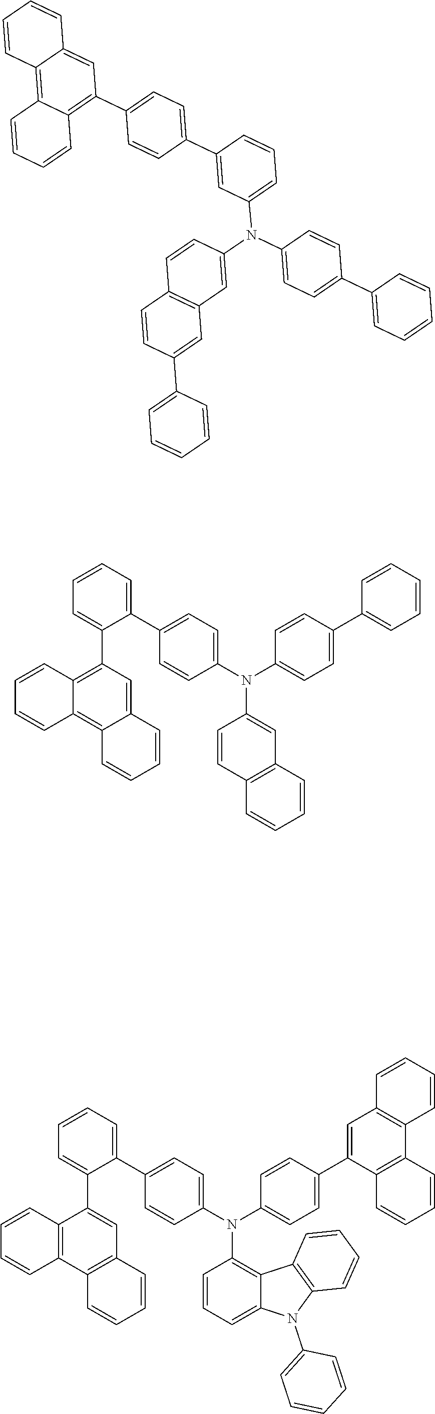 [SMILES: c1ccc(-c2ccc(N(c3cccc(-c4ccc(-c5cc6ccccc6c6ccccc56)cc4)c3)c3ccc4ccc(-c5ccccc5)cc4c3)cc2)cc1 ; c1ccc(-c2ccc(N(c3ccc(-c4ccccc4-c4cc5ccccc5c5ccccc45)cc3)c3ccc4ccccc4c3)cc2)cc1 ; c1ccc(-n2c3ccccc3c3c(N(c4ccc(-c5ccccc5-c5cc6ccccc6c6ccccc56)cc4)c4ccc(-c5cc6ccccc6c6ccccc56)cc4)cccc32)cc1] 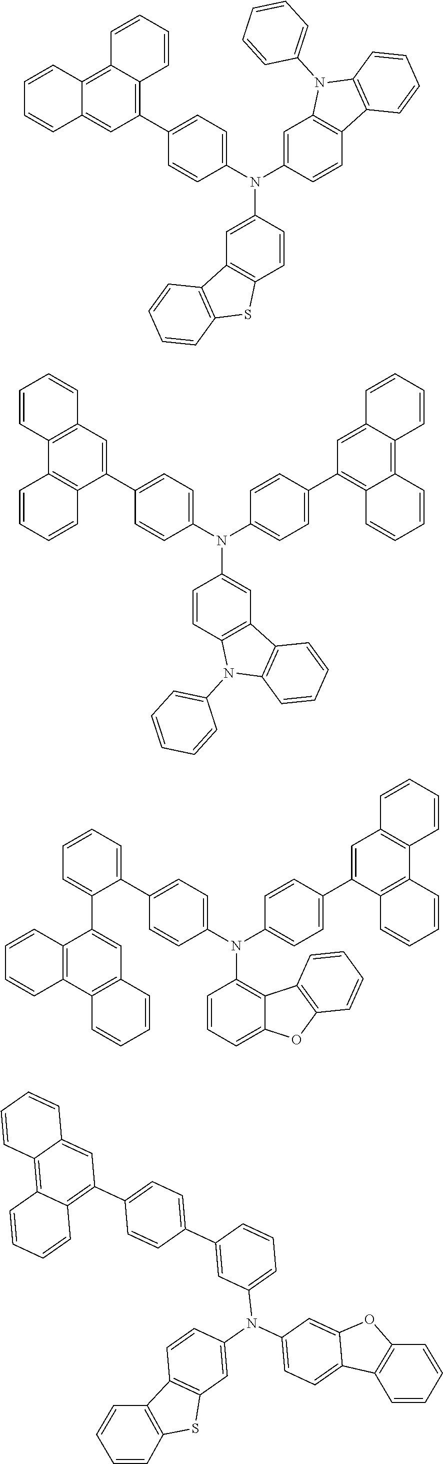 [SMILES: c1ccc(-n2c3ccccc3c3ccc(N(c4ccc(-c5cc6ccccc6c6ccccc56)cc4)c4ccc5sc6ccccc6c5c4)cc32)cc1 ; c1ccc(-n2c3ccccc3c3cc(N(c4ccc(-c5cc6ccccc6c6ccccc56)cc4)c4ccc(-c5cc6ccccc6c6ccccc56)cc4)ccc32)cc1 ; c1ccc(-c2cc3ccccc3c3ccccc23)c(-c2ccc(N(c3ccc(-c4cc5ccccc5c5ccccc45)cc3)c3cccc4oc5ccccc5c34)cc2)c1 ; c1cc(-c2ccc(-c3cc4ccccc4c4ccccc34)cc2)cc(N(c2ccc3c(c2)oc2ccccc23)c2ccc3c(c2)sc2ccccc23)c1] 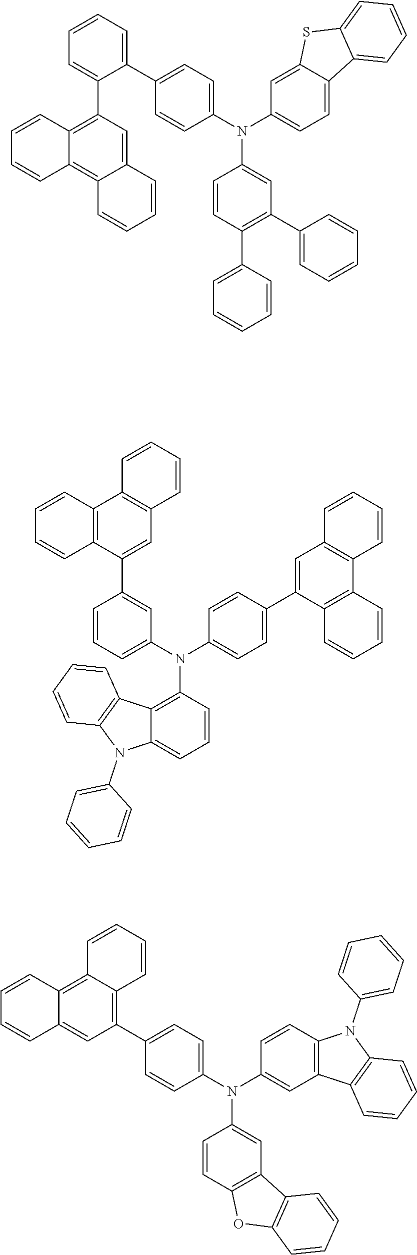 [SMILES: c1ccc(-c2ccc(N(c3ccc(-c4ccccc4-c4cc5ccccc5c5ccccc45)cc3)c3ccc4c(c3)sc3ccccc34)cc2-c2ccccc2)cc1 ; c1ccc(-n2c3ccccc3c3c(N(c4ccc(-c5cc6ccccc6c6ccccc56)cc4)c4cccc(-c5cc6ccccc6c6ccccc56)c4)cccc32)cc1 ; c1ccc(-n2c3ccccc3c3cc(N(c4ccc(-c5cc6ccccc6c6ccccc56)cc4)c4ccc5oc6ccccc6c5c4)ccc32)cc1] 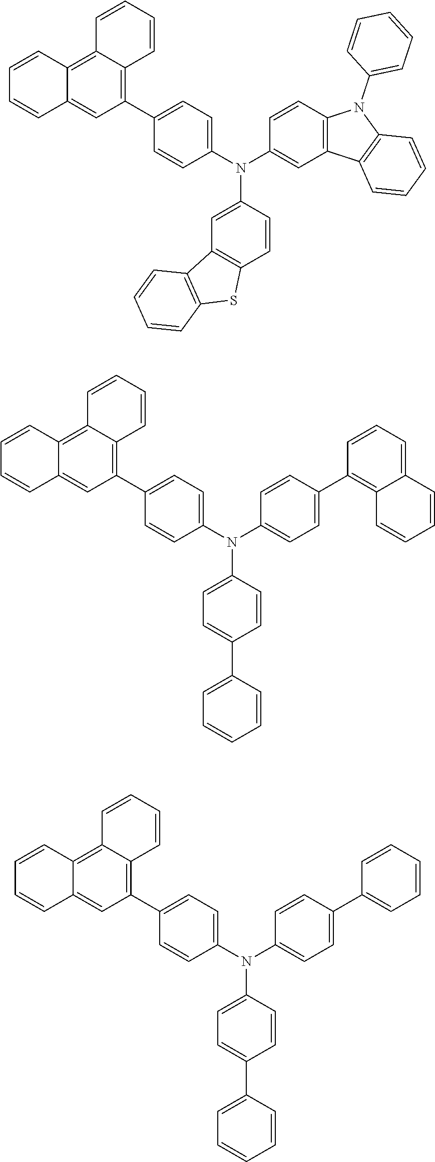 [SMILES: c1ccc(-n2c3ccccc3c3cc(N(c4ccc(-c5cc6ccccc6c6ccccc56)cc4)c4ccc5sc6ccccc6c5c4)ccc32)cc1 ; c1ccc(-c2ccc(N(c3ccc(-c4cccc5ccccc45)cc3)c3ccc(-c4cc5ccccc5c5ccccc45)cc3)cc2)cc1 ; c1ccc(-c2ccc(N(c3ccc(-c4ccccc4)cc3)c3ccc(-c4cc5ccccc5c5ccccc45)cc3)cc2)cc1] 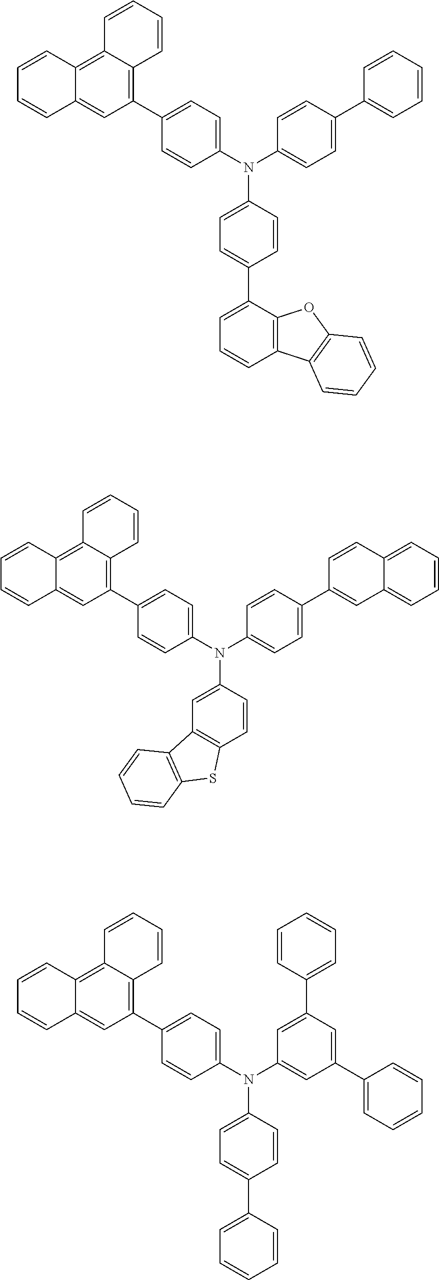 [SMILES: c1ccc(-c2ccc(N(c3ccc(-c4cc5ccccc5c5ccccc45)cc3)c3ccc(-c4cccc5c4oc4ccccc45)cc3)cc2)cc1 ; c1ccc2cc(-c3ccc(N(c4ccc(-c5cc6ccccc6c6ccccc56)cc4)c4ccc5sc6ccccc6c5c4)cc3)ccc2c1 ; c1ccc(-c2ccc(N(c3ccc(-c4cc5ccccc5c5ccccc45)cc3)c3cc(-c4ccccc4)cc(-c4ccccc4)c3)cc2)cc1] 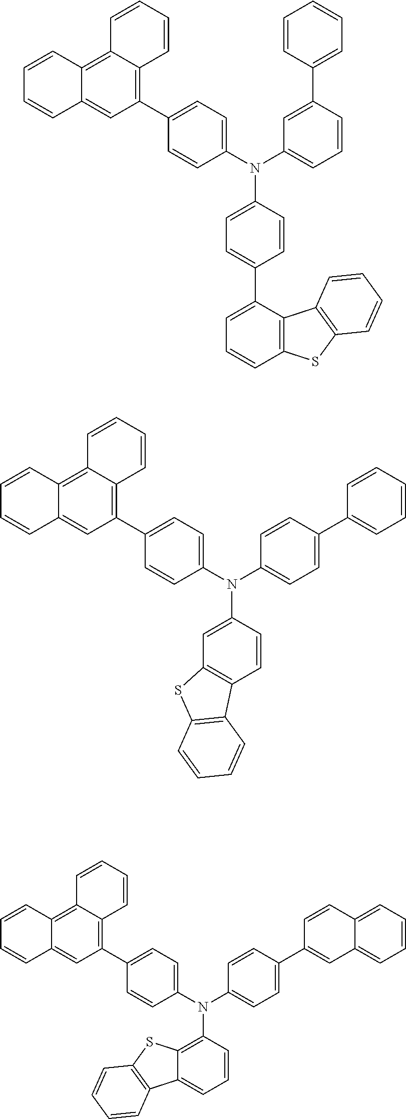 [SMILES: c1ccc(-c2cccc(N(c3ccc(-c4cc5ccccc5c5ccccc45)cc3)c3ccc(-c4cccc5sc6ccccc6c45)cc3)c2)cc1 ; c1ccc(-c2ccc(N(c3ccc(-c4cc5ccccc5c5ccccc45)cc3)c3ccc4c(c3)sc3ccccc34)cc2)cc1 ; c1ccc2cc(-c3ccc(N(c4ccc(-c5cc6ccccc6c6ccccc56)cc4)c4cccc5c4sc4ccccc45)cc3)ccc2c1] 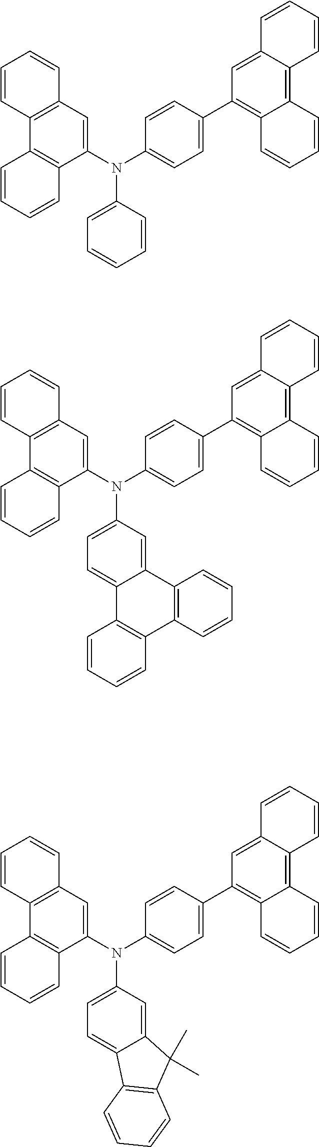 [SMILES: c1ccc(N(c2ccc(-c3cc4ccccc4c4ccccc34)cc2)c2cc3ccccc3c3ccccc23)cc1 ; c1ccc2c(c1)cc(-c1ccc(N(c3ccc4c5ccccc5c5ccccc5c4c3)c3cc4ccccc4c4ccccc34)cc1)c1ccccc12 ; CC1(C)c2ccccc2-c2ccc(N(c3ccc(-c4cc5ccccc5c5ccccc45)cc3)c3cc4ccccc4c4ccccc34)cc21] 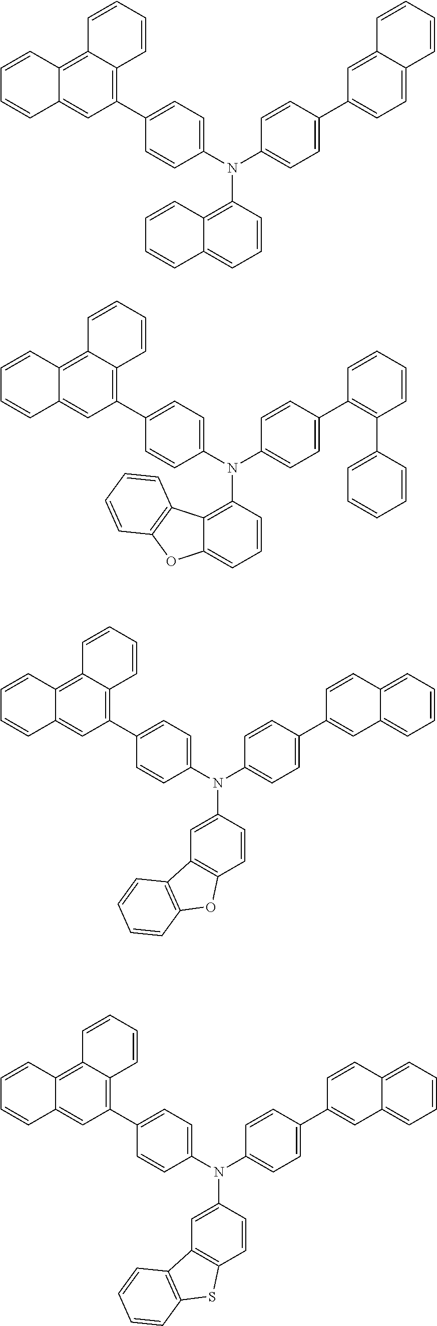 [SMILES: c1ccc2cc(-c3ccc(N(c4ccc(-c5cc6ccccc6c6ccccc56)cc4)c4cccc5ccccc45)cc3)ccc2c1 ; c1ccc(-c2ccccc2-c2ccc(N(c3ccc(-c4cc5ccccc5c5ccccc45)cc3)c3cccc4oc5ccccc5c34)cc2)cc1 ; c1ccc2cc(-c3ccc(N(c4ccc(-c5cc6ccccc6c6ccccc56)cc4)c4ccc5oc6ccccc6c5c4)cc3)ccc2c1 ; c1ccc2cc(-c3ccc(N(c4ccc(-c5cc6ccccc6c6ccccc56)cc4)c4ccc5sc6ccccc6c5c4)cc3)ccc2c1] 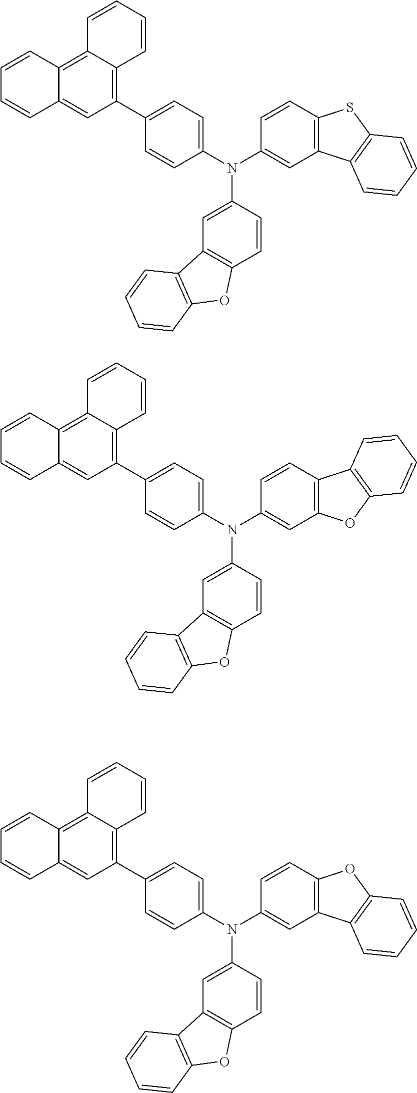 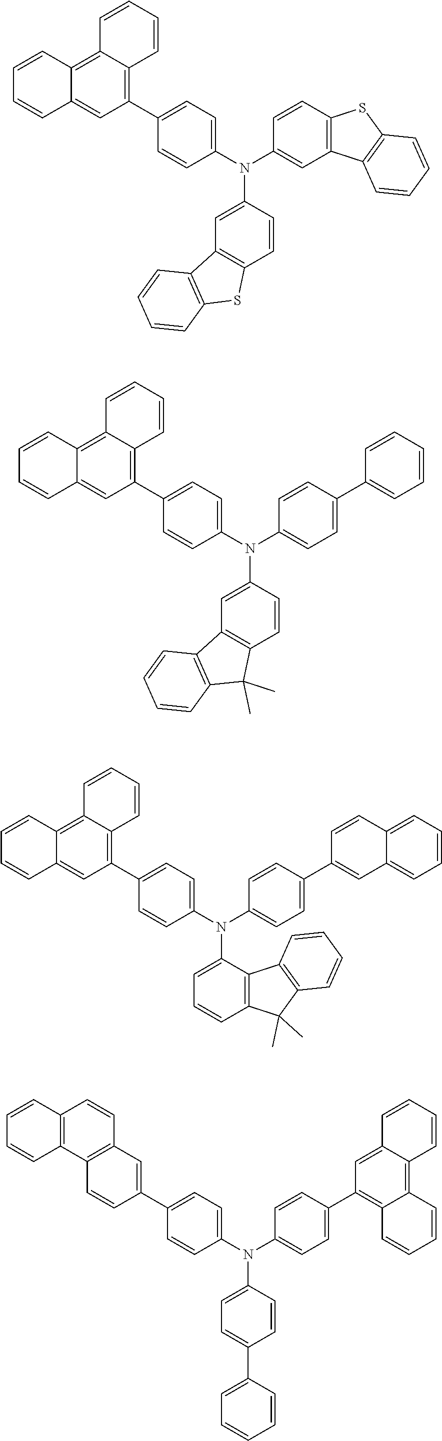 [SMILES: c1ccc2c(c1)cc(-c1ccc(N(c3ccc4sc5ccccc5c4c3)c3ccc4sc5ccccc5c4c3)cc1)c1ccccc12 ; CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2c(N(c3ccc(-c4ccc5ccccc5c4)cc3)c3ccc(-c4cc5ccccc5c5ccccc45)cc3)cccc21 ; c1ccc(-c2ccc(N(c3ccc(-c4ccc5c(ccc6ccccc65)c4)cc3)c3ccc(-c4cc5ccccc5c5ccccc45)cc3)cc2)cc1] 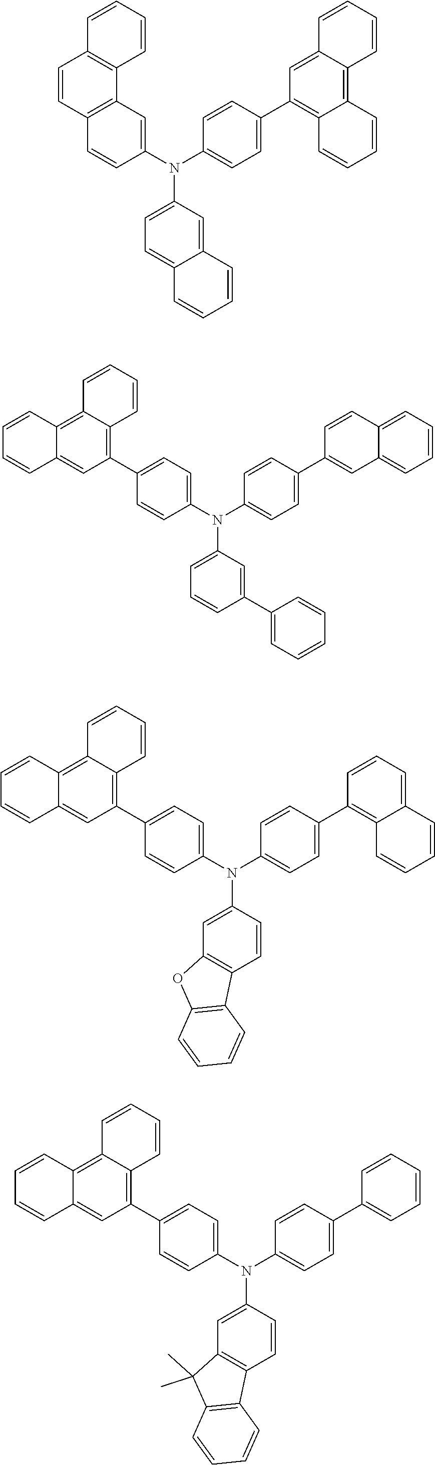 [SMILES: c1ccc2cc(N(c3ccc(-c4cc5ccccc5c5ccccc45)cc3)c3ccc4ccc5ccccc5c4c3)ccc2c1 ; c1ccc(-c2cccc(N(c3ccc(-c4ccc5ccccc5c4)cc3)c3ccc(-c4cc5ccccc5c5ccccc45)cc3)c2)cc1 ; c1ccc2c(-c3ccc(N(c4ccc(-c5cc6ccccc6c6ccccc56)cc4)c4ccc5c(c4)oc4ccccc45)cc3)cccc2c1 ; CC1(C)c2ccccc2-c2ccc(N(c3ccc(-c4ccccc4)cc3)c3ccc(-c4cc5ccccc5c5ccccc45)cc3)cc21] 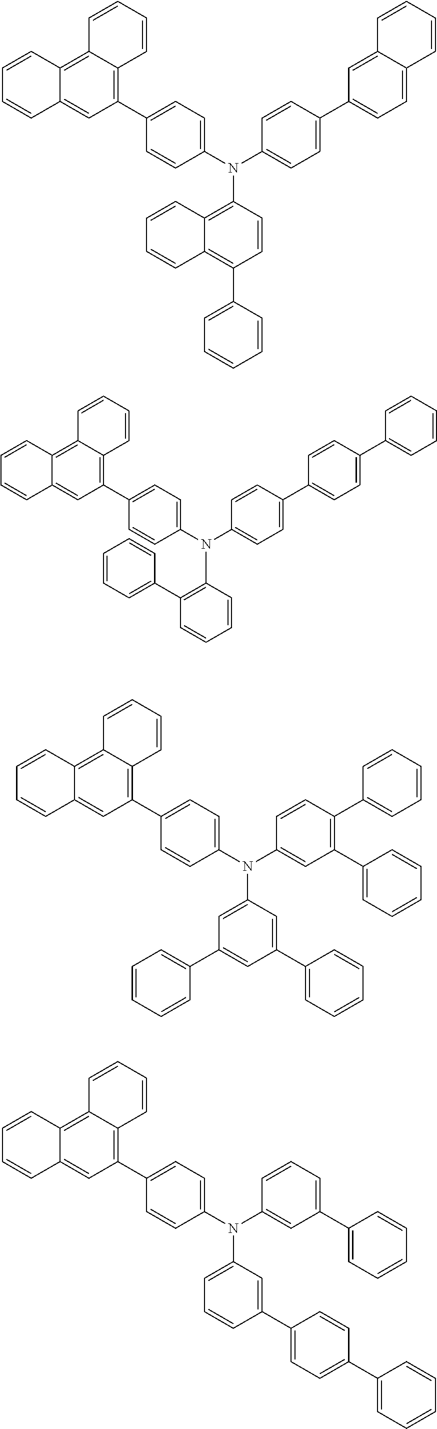 [SMILES: c1ccc(-c2ccc(N(c3ccc(-c4ccc5ccccc5c4)cc3)c3ccc(-c4cc5ccccc5c5ccccc45)cc3)c3ccccc23)cc1 ; c1ccc(-c2ccc(-c3ccc(N(c4ccc(-c5cc6ccccc6c6ccccc56)cc4)c4ccccc4-c4ccccc4)cc3)cc2)cc1 ; c1ccc(-c2cc(-c3ccccc3)cc(N(c3ccc(-c4cc5ccccc5c5ccccc45)cc3)c3ccc(-c4ccccc4)c(-c4ccccc4)c3)c2)cc1 ; c1ccc(-c2ccc(-c3cccc(N(c4ccc(-c5cc6ccccc6c6ccccc56)cc4)c4cccc(-c5ccccc5)c4)c3)cc2)cc1] 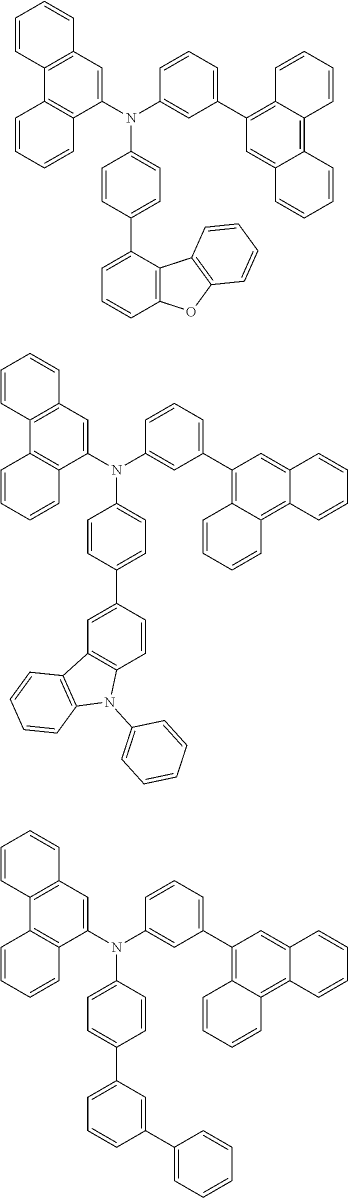 [SMILES: c1cc(-c2cc3ccccc3c3ccccc23)cc(N(c2ccc(-c3cccc4oc5ccccc5c34)cc2)c2cc3ccccc3c3ccccc23)c1 ; c1ccc(-n2c3ccccc3c3cc(-c4ccc(N(c5cccc(-c6cc7ccccc7c7ccccc67)c5)c5cc6ccccc6c6ccccc56)cc4)ccc32)cc1 ; c1ccc(-c2cccc(-c3ccc(N(c4cccc(-c5cc6ccccc6c6ccccc56)c4)c4cc5ccccc5c5ccccc45)cc3)c2)cc1] 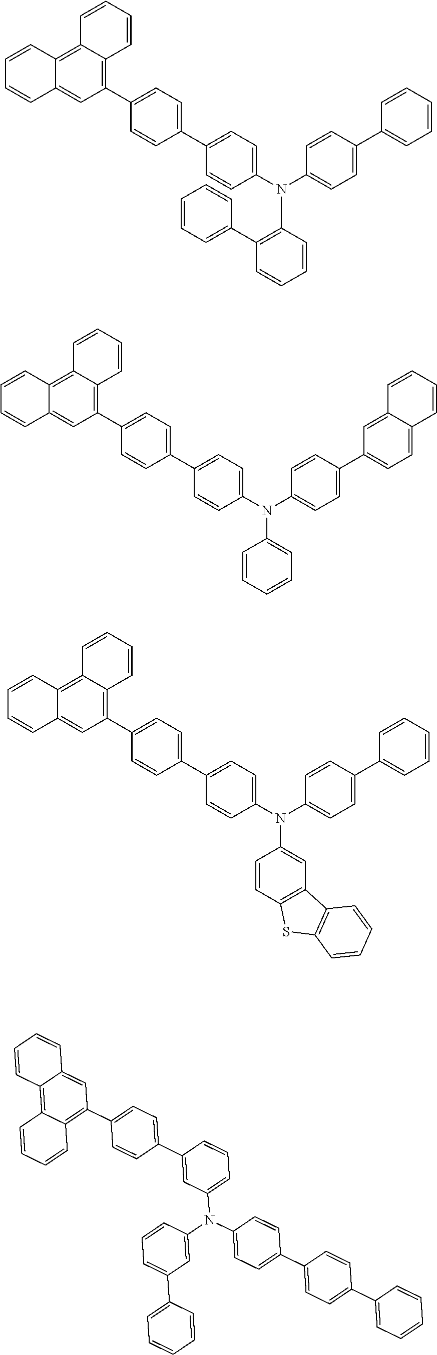 [SMILES: c1ccc(-c2ccc(N(c3ccc(-c4ccc(-c5cc6ccccc6c6ccccc56)cc4)cc3)c3ccccc3-c3ccccc3)cc2)cc1 ; c1ccc(N(c2ccc(-c3ccc(-c4cc5ccccc5c5ccccc45)cc3)cc2)c2ccc(-c3ccc4ccccc4c3)cc2)cc1 ; c1ccc(-c2ccc(N(c3ccc(-c4ccc(-c5cc6ccccc6c6ccccc56)cc4)cc3)c3ccc4sc5ccccc5c4c3)cc2)cc1 ; c1ccc(-c2ccc(-c3ccc(N(c4cccc(-c5ccccc5)c4)c4cccc(-c5ccc(-c6cc7ccccc7c7ccccc67)cc5)c4)cc3)cc2)cc1] 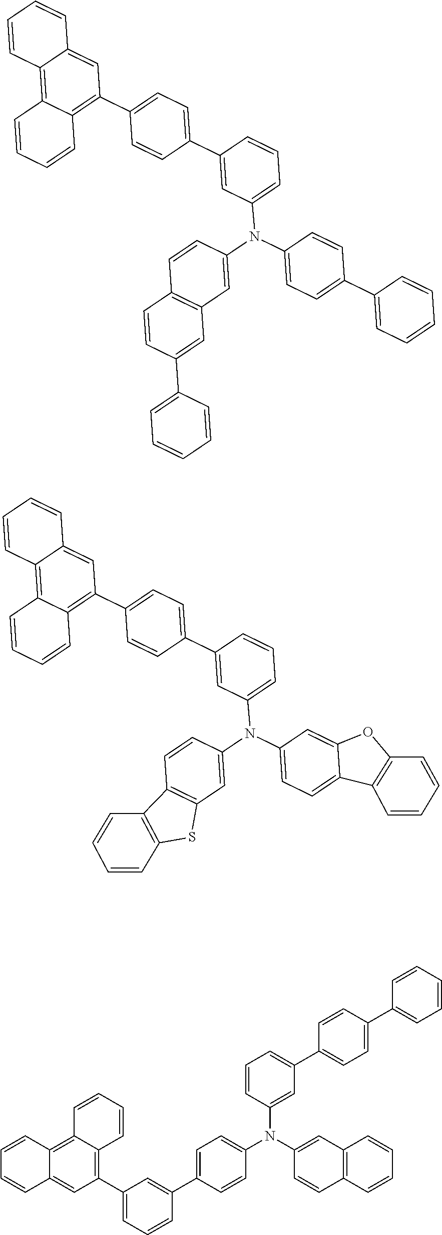 [SMILES: c1ccc(-c2ccc(N(c3cccc(-c4ccc(-c5cc6ccccc6c6ccccc56)cc4)c3)c3ccc4ccc(-c5ccccc5)cc4c3)cc2)cc1 ; c1cc(-c2ccc(-c3cc4ccccc4c4ccccc34)cc2)cc(N(c2ccc3c(c2)oc2ccccc23)c2ccc3c(c2)sc2ccccc23)c1 ; c1ccc(-c2ccc(-c3cccc(N(c4ccc(-c5cccc(-c6cc7ccccc7c7ccccc67)c5)cc4)c4ccc5ccccc5c4)c3)cc2)cc1] 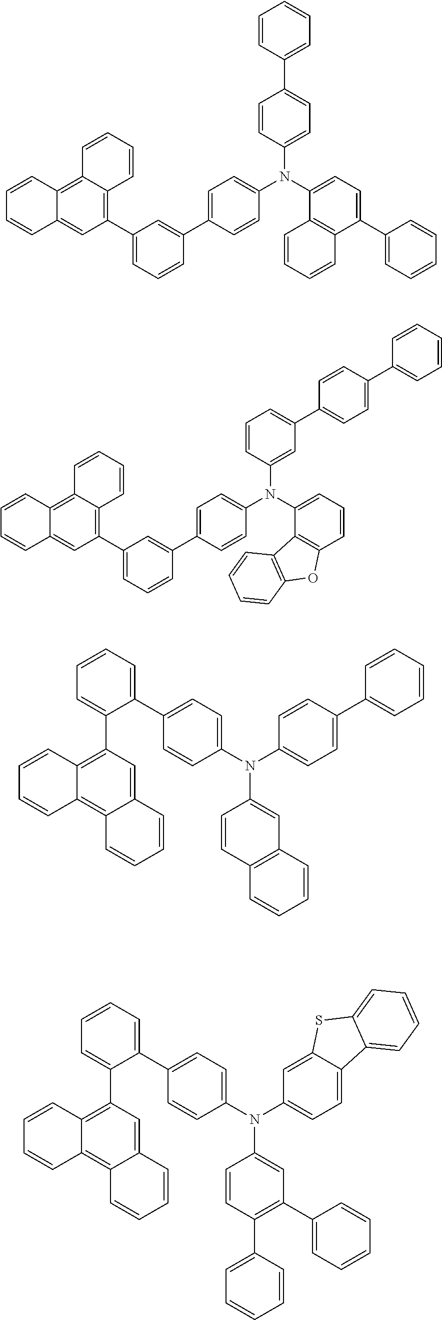 [SMILES: c1ccc(-c2ccc(N(c3ccc(-c4cccc(-c5cc6ccccc6c6ccccc56)c4)cc3)c3ccc(-c4ccccc4)c4ccccc34)cc2)cc1 ; c1ccc(-c2ccc(-c3cccc(N(c4ccc(-c5cccc(-c6cc7ccccc7c7ccccc67)c5)cc4)c4cccc5oc6ccccc6c45)c3)cc2)cc1 ; c1ccc(-c2ccc(N(c3ccc(-c4ccccc4-c4cc5ccccc5c5ccccc45)cc3)c3ccc4ccccc4c3)cc2)cc1 ; c1ccc(-c2ccc(N(c3ccc(-c4ccccc4-c4cc5ccccc5c5ccccc45)cc3)c3ccc4c(c3)sc3ccccc34)cc2-c2ccccc2)cc1] 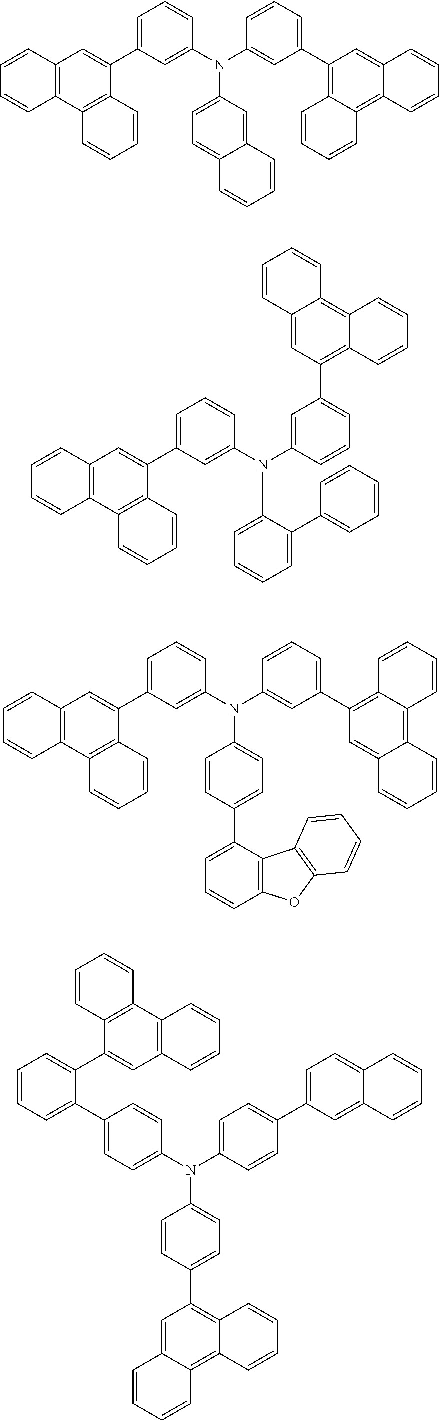 [SMILES: c1cc(-c2cc3ccccc3c3ccccc23)cc(N(c2cccc(-c3cc4ccccc4c4ccccc34)c2)c2ccc3ccccc3c2)c1 ; c1ccc(-c2ccccc2N(c2cccc(-c3cc4ccccc4c4ccccc34)c2)c2cccc(-c3cc4ccccc4c4ccccc34)c2)cc1 ; c1cc(-c2cc3ccccc3c3ccccc23)cc(N(c2ccc(-c3cccc4oc5ccccc5c34)cc2)c2cccc(-c3cc4ccccc4c4ccccc34)c2)c1 ; c1ccc(-c2cc3ccccc3c3ccccc23)c(-c2ccc(N(c3ccc(-c4ccc5ccccc5c4)cc3)c3ccc(-c4cc5ccccc5c5ccccc45)cc3)cc2)c1] 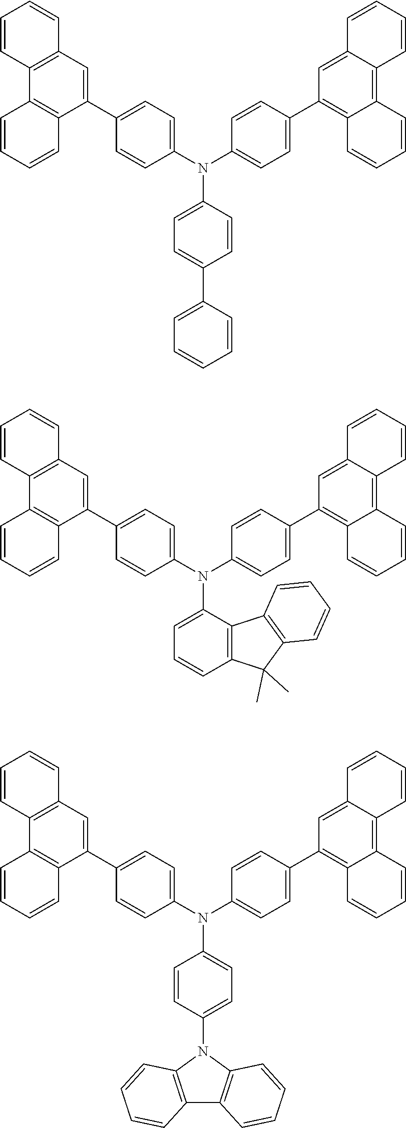 [SMILES: c1ccc(-c2ccc(N(c3ccc(-c4cc5ccccc5c5ccccc45)cc3)c3ccc(-c4cc5ccccc5c5ccccc45)cc3)cc2)cc1 ; CC1(C)c2ccccc2-c2c(N(c3ccc(-c4cc5ccccc5c5ccccc45)cc3)c3ccc(-c4cc5ccccc5c5ccccc45)cc3)cccc21 ; c1ccc2c(c1)cc(-c1ccc(N(c3ccc(-c4cc5ccccc5c5ccccc45)cc3)c3ccc(-n4c5ccccc5c5ccccc54)cc3)cc1)c1ccccc12] 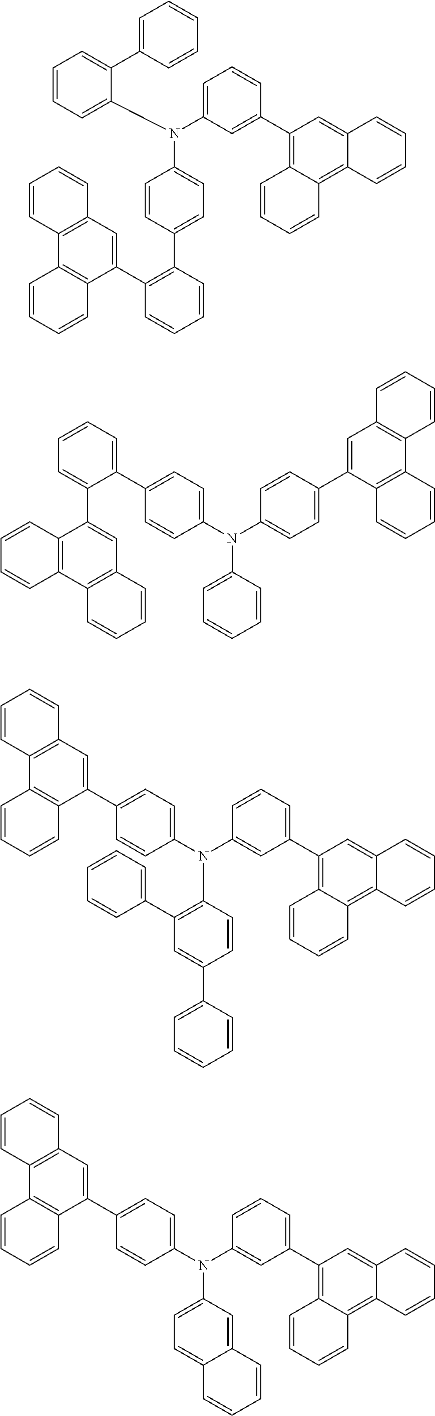 [SMILES: c1ccc(-c2ccccc2N(c2ccc(-c3ccccc3-c3cc4ccccc4c4ccccc34)cc2)c2cccc(-c3cc4ccccc4c4ccccc34)c2)cc1 ; c1ccc(N(c2ccc(-c3ccccc3-c3cc4ccccc4c4ccccc34)cc2)c2ccc(-c3cc4ccccc4c4ccccc34)cc2)cc1 ; c1ccc(-c2ccc(N(c3ccc(-c4cc5ccccc5c5ccccc45)cc3)c3cccc(-c4cc5ccccc5c5ccccc45)c3)c(-c3ccccc3)c2)cc1 ; c1cc(-c2cc3ccccc3c3ccccc23)cc(N(c2ccc(-c3cc4ccccc4c4ccccc34)cc2)c2ccc3ccccc3c2)c1] 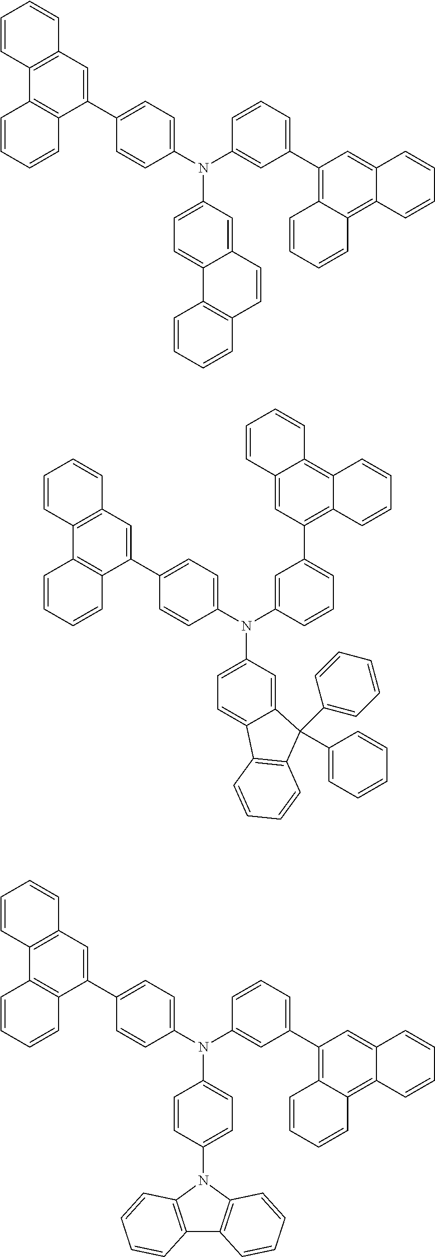 [SMILES: c1cc(-c2cc3ccccc3c3ccccc23)cc(N(c2ccc(-c3cc4ccccc4c4ccccc34)cc2)c2ccc3c(ccc4ccccc43)c2)c1 ; c1ccc(C2(c3ccccc3)c3ccccc3-c3ccc(N(c4ccc(-c5cc6ccccc6c6ccccc56)cc4)c4cccc(-c5cc6ccccc6c6ccccc56)c4)cc32)cc1 ; c1cc(-c2cc3ccccc3c3ccccc23)cc(N(c2ccc(-c3cc4ccccc4c4ccccc34)cc2)c2ccc(-n3c4ccccc4c4ccccc43)cc2)c1] 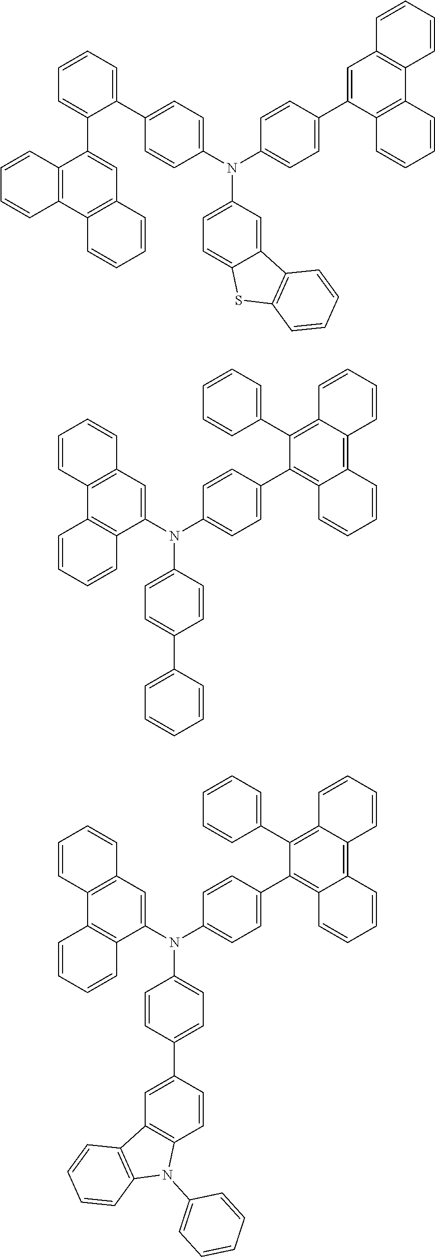 [SMILES: c1ccc(-c2cc3ccccc3c3ccccc23)c(-c2ccc(N(c3ccc(-c4cc5ccccc5c5ccccc45)cc3)c3ccc4sc5ccccc5c4c3)cc2)c1 ; c1ccc(-c2ccc(N(c3ccc(-c4c(-c5ccccc5)c5ccccc5c5ccccc45)cc3)c3cc4ccccc4c4ccccc34)cc2)cc1 ; c1ccc(-c2c(-c3ccc(N(c4ccc(-c5ccc6c(c5)c5ccccc5n6-c5ccccc5)cc4)c4cc5ccccc5c5ccccc45)cc3)c3ccccc3c3ccccc23)cc1] 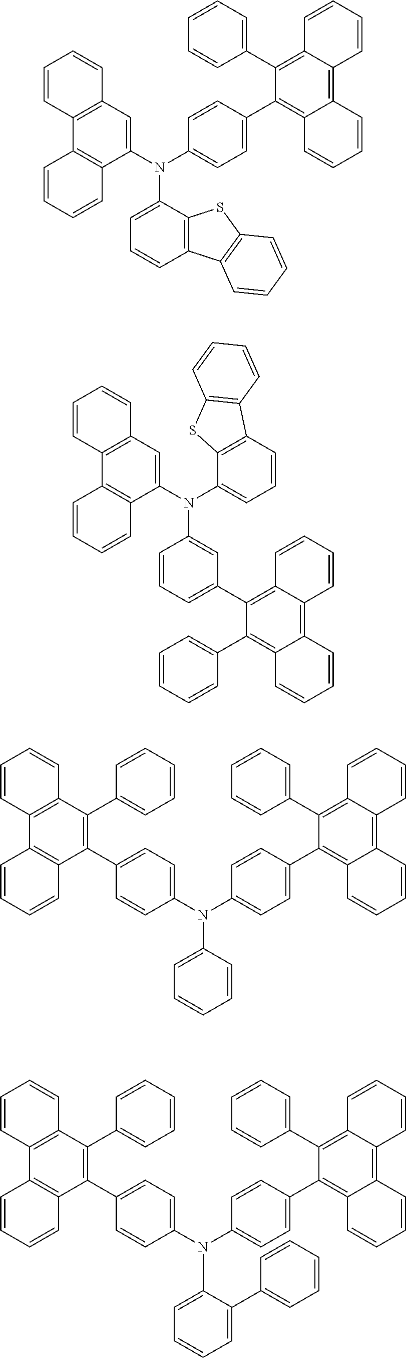 [SMILES: c1ccc(-c2c(-c3ccc(N(c4cc5ccccc5c5ccccc45)c4cccc5c4sc4ccccc45)cc3)c3ccccc3c3ccccc23)cc1 ; c1ccc(-c2c(-c3cccc(N(c4cc5ccccc5c5ccccc45)c4cccc5c4sc4ccccc45)c3)c3ccccc3c3ccccc23)cc1 ; c1ccc(-c2c(-c3ccc(N(c4ccccc4)c4ccc(-c5c(-c6ccccc6)c6ccccc6c6ccccc56)cc4)cc3)c3ccccc3c3ccccc23)cc1 ; c1ccc(-c2ccccc2N(c2ccc(-c3c(-c4ccccc4)c4ccccc4c4ccccc34)cc2)c2ccc(-c3c(-c4ccccc4)c4ccccc4c4ccccc34)cc2)cc1] 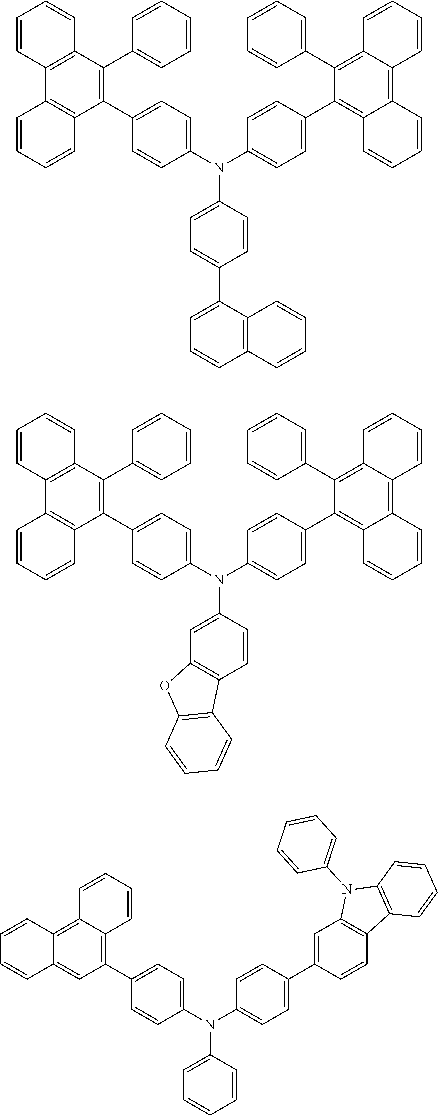 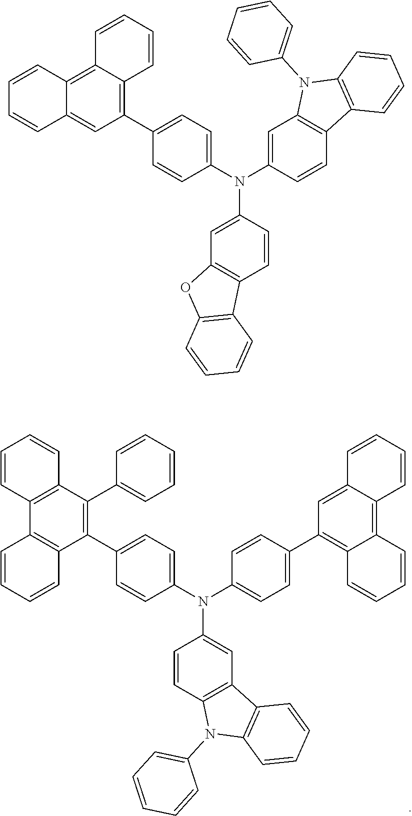 [SMILES: c1ccc(-n2c3ccccc3c3ccc(N(c4ccc(-c5cc6ccccc6c6ccccc56)cc4)c4ccc5c(c4)oc4ccccc45)cc32)cc1 ; c1ccc(-c2c(-c3ccc(N(c4ccc(-c5cc6ccccc6c6ccccc56)cc4)c4ccc5c(c4)c4ccccc4n5-c4ccccc4)cc3)c3ccccc3c3ccccc23)cc1]
